(19) United States
(12) Patent Application Publication
(10) Pub. No.: US 2003/0165891 A1
(43) Pub. Date: Sep. 4, 2003

(54) Novel TWIK-6, TWIK-7, IC23927, TWIK-8, IC47611, IC47615, HNMDA-1, TWIK-9 alpha2delta-4, 54414, and 53763 molecules and uses therefor

(75) Inventors: Rory A.J. Curtis, Framingham, MA (US); Maria Alexandra Glucksmann, Lexington, MA (US); Inmaculada Silos-Santiago, Cambridge, MA (US)

Correspondence Address: LAHIVE & COCKFIELD, 28 STATE STREET, BOSTON, MA, 02109, US

(73) Assignee: Millennium Pharmaceuticals, Inc., Cambridge, MA (US)

(21) Appl. No.: 10/146,733

(22) Filed: May 15, 2002

Related U.S. Application Data
(63) Continuation of application No. 09/518,866, filed on Mar. 3, 2000, now abandoned.
(63) Continuation of application No. 09/515,520, filed on Feb. 29, 2000.
(63) Continuation of application No. 09/796,720, filed on Feb. 28, 2001.
(63) Continuation of application No. 09/828,035, filed on Apr. 6, 2001.
(63) Continuation of application No. 09/833,081, filed on Apr. 11, 2001.
(63) Continuation of application No. 09/843,128, filed on Apr. 25, 2001.
(63) Continuation of application No. 09/957,683, filed on Sep. 19, 2001.
(63) Continuation of application No. 09/964,252, filed on Sep. 25, 2001.
(63) Continuation of application No. 09/964,256, filed on Sep. 25, 2001.
(63) Continuation of application No. 10/024,623, filed on Dec. 17, 2001.
(60) Provisional application No. 60/185,938, filed on Feb. 29, 2000.
(60) Provisional application No. 60/195,734, filed on Apr. 7, 2000.
(60) Provisional application No. 60/195,993, filed on Apr. 11, 2000.
(60) Provisional application No. 60/199,799, filed on Apr. 26, 2000.
(60) Provisional application No. 60/233,537, filed on Sep. 19, 2000.
(60) Provisional application No. 60/235,059, filed on Sep. 25, 2000.
(60) Provisional application No. 60/235,018, filed on Sep. 25, 2000.
(60) Provisional application No. 60/256,240, filed on Dec. 15, 2000.
(60) Provisional application No. 60/256,588, filed on Dec. 18, 2000.
(60) Provisional application No. 60/258,028, filed on Dec. 21, 2000.

(51) Int. Cl. C12Q 1/68; G01N 33/53; G01N 33/567; C07H 21/04; C12N 9/12; C12P 21/02; C12N 5/06; C07K 14/705; C07K 16/28

(52) U.S. Cl. 435/6; 435/69.1; 435/194; 435/320.1; 435/325; 530/350; 536/23.2; 530/388.22; 435/7.2

(57) ABSTRACT

The invention provides isolated nucleic acids molecules, designated TWIK-6, TWIK-7, IC23927, TWIK-8, IC47611, IC47615, HNMDA-1, TWIK-9, α₂δ-4, 54414, and 53763 nucleic acid molecules, which encode novel ion channel family molecules, including calcium channels, potassium channels, and NMDA receptors. The invention also provides antisense nucleic acid molecules, recombinant expression vectors containing TWIK-6, TWIK-7, IC23927, TWIK-8, IC47611, IC47615, HNMDA-1, TWIK-9, α₂δ-4, 54414, and 53763 nucleic acid molecules, host cells into which the expression vectors have been introduced, and non-human transgenic animals in which a TWIK-6, TWIK-7, IC23927, TWIK-8, IC47611, IC47615, HNMDA-1, TWIK-9, α₂δ-4, 54414, or 53763 gene has been introduced or disrupted. The invention still further provides isolated TWIK-6, TWIK-7, IC23927, TWIK-8, IC47611, IC47615, HNMDA-1, TWIK-9, α₂δ-4, 54414, and 53763 polypeptides, fusion polypeptides, antigenic peptides and anti-TWIK-6, TWIK-7, IC23927, TWIK-8, IC47611, IC47615, HNMDA-1, TWIK-9, α₂δ-4, 54414, and 53763 antibodies. Diagnostic and therapeutic methods utilizing compositions of the invention are also provided.

### RELATED APPLICATIONS

This application is a continuation-in-part of U.S. patent application Ser. No. 09/518,866, filed Mar. 3, 2000 (pending).

This application is also a continuation-in-part of U.S. patent application Ser. No. 09/515,520, filed Feb. 29, 2000 (pending).

This application is also a continuation-in-part of U.S. patent application Ser. No. 09/796,720, filed Feb. 28, 2001 (pending), which claims the benefit of U.S. Provisional Application Serial No. 60/185,938, filed Feb. 29, 2000.

This application is also a continuation-in-part of U.S. patent application Ser. No. 09/828,035, filed Apr. 6, 2001 (pending), which claims the benefit of U.S. Provisional Application Serial No. 60/195,734, filed Apr. 7, 2000.

This application is also a continuation-in-part of U.S. patent application Ser. No. 09/833,081, filed Apr. 11, 2001 (pending), which claims the benefit of U.S. Provisional Application Serial No. 60/195,993, filed Apr. 11, 2000.

This application is also a continuation-in-part of U.S. patent application Ser. No. 09/843,128, filed Apr. 25, 2001 (pending), which claims the benefit of U.S. Provisional Application Serial No. 60/199,799, filed Apr. 26, 2000.

This application is also a continuation-in-part of U.S. patent application Ser. No. 09/957,683, filed Sep. 19, 2001 (pending), which claims the benefit of U.S. Provisional Application Serial No. 60/233,537, filed Sep. 19, 2000.

This application is also a continuation-in-part of U.S. patent application Ser. No. 09/964,252, filed Sep. 25, 2001 (pending), which claims the benefit of U.S. Provisional Application Serial No. 60/235,059, filed Sep. 25, 2000.

This application is also a continuation-in-part of U.S. patent application Ser. No. 09/964,256, filed Sep. 25, 2001 (pending), which claims the benefit of U.S. Provisional Application Serial No. 60/235,018, filed Sep. 25, 2000.

This application is also a continuation-in-part of U.S. patent application Ser. No. 10/024,623, filed Dec. 17, 2001 (pending), which claims the benefit of U.S. Provisional Application Serial No. 60/256,240, filed Dec. 15, 2000, U.S. Provisional Application Serial No. 60/256,588, filed Dec. 18, 2000, and U.S. Provisional Application Serial No. 60/258,028, filed Dec. 21, 2000.

The entire contents of each of the above-referenced patent applications are incorporated herein by this reference.

INDEX

| Chapter | Page | Title |
|---|---|---|
| I. | 2 | A NOVEL POTASSIUM CHANNEL MOLECULE |
| | | AND USES THEREFOR |
| II. | 86 | NUCLEIC ACIDS ENCODING TWIK-7, A NOVEL |
| | | POTASSIUM CHANNEL PROTEIN |
| III. | 160 | 23927, A NOVEL HUMAN ION CHANNEL |
| IV. | 244 | 12303, A NOVEL HUMAN TWIK MOLECULE AND |
| | | USES THEREOF |
| V. | 325 | 47611, A NOVEL HUMAN ION CHANNEL AND |
| | | USES THEREOF |
| VI. | 402 | 47615, A NOVEL HUMAN ION CHANNEL AND |
| | | USES THEREOF |
| VII. | 477 | 55063, A NOVEL HUMAN NMDA FAMILY |
| | | MEMBER AND USES THEREOF |
| VIII. | 553 | 56115, A NOVEL HUMAN TWIK POTASSIUM |
| | | CHANNEL AND USES THEREFOR |
| IX. | 635 | 25658, A NOVEL HUMAN CALCIUM CHANNEL |
| | | SUBUNIT AND USES THEREOF |
| X. | 713 | 8099, 46455, 54414, 53763, 67076, 67102, 44181, |
| | | 67084FL, AND 67084 ALT, HUMAN PROTEINS AND |
| | | METHODS OF USE THEREOF |

### I. A NOVEL POTASSIUM CHANNEL MOLECULE AND USES THEREFOR

### Background of the Invention

Potassium (K⁺) channels are ubiquitous proteins which are involved in the setting of the resting membrane potential as well as in the modulation of the electrical activity of cells. In excitable cells, K⁺ channels influence action potential waveforms, firing frequency, and neurotransmitter secretion (Rudy, B. (1988) Neuroscience, 25, 729-749; Hille, B. (1992) Ionic Channels of Excitable Membranes, 2nd Ed.). In non-excitable cells, they are involved in hormone secretion, cell volume regulation and potentially in cell proliferation and differentiation (Lewis et al. (1995) Annu. Rev. Immunol., 13, 623-653). Developments in electrophysiology have allowed the identification and the characterization of an astonishing variety of K⁺ channels that differ in their biophysical properties, pharmacology, regulation and tissue distribution (Rudy, B. (1988) Neuroscience, 25, 729-749; Hille, B. (1992) Ionic Channels of Excitable Membranes, 2nd Ed.). More recently, cloning efforts have shed considerable light on the mechanisms that determine this functional diversity. Furthermore, analyses of structure-function relationships have provided an important set of data concerning the molecular basis of the biophysical properties (selectivity, gating, assembly) and the pharmacological properties of cloned K⁺ channels.

Functional diversity of K⁺ channels arises mainly from the existence of a great number of genes coding for pore-forming subunits, as well as for other associated regulatory subunits. Two main structural families of pore-forming subunits have been identified. The first one consists of subunits with a conserved hydrophobic core containing six transmembrane domains (TMDs). These K⁺ channel α subunits participate in the formation of outward rectifier voltage-gated (Kv) and Ca²⁺-dependent K⁺ channels. The fourth TMD contains repeated positive charges involved in the voltage gating of these channels and hence in their outward rectification (Logothetis et al. (1992) Neuron, 8, 531-540; Bezanilla et al. (1994) Biophys. J. 66, 1011-1021).

The second family of pore-forming subunits have only two TMDs. They are essential subunits of inward-rectifying (IRK), G-protein-coupled (GIRK) and ATP-sensitive (K_{ATP}) K⁺ channels. The inward rectification results from a voltage-dependent block by cytoplasmic Mg²⁺ and polyamines (Matsuda, H. (1991) Annu. Rev. Physiol., 53, 289-298). A conserved domain, called the P domain, is present in all members of both families (Pongs, O. (1993) J. Membr. Biol., 136, 1-8; Heginbotham et al. (1994) Biophys. J. 66,1061-1067; Mackinnon, R. (1995) Neuron, 14, 889-892; Pascual et al., (1995) Neuron., and 14, 1055-1063). This domain is an essential element of the aqueous K⁺-selective pore. In both groups, the assembly of four subunits is necessary to form a functional K⁺ channel (Mackinnon, R. (1991) Nature, 350, 232-235; Yang et al., (1995) Neuron, 15, 1441-1447.

In both six TMD and two TMD pore-forming subunit families, different subunits coded by different genes can associate to form heterotetramers with new channel properties (Isacoff et al., (1990) Nature, 345, 530-534). A selective formation of heteropolymeric channels may allow each cell to develop the best K⁺ current repertoire suited to its function. Pore-forming α subunits of Kv channels are classified into different subfamilies according to their sequence similarity (Chandy et al. (1993) Trends Pharmacol. Sci., 14, 434). Tetramerization is believed to occur preferentially between members of each subgroup (Covarrubias et al. (1991) Neuron, 7, 763-773). The domain responsible for this selective association is localized in the N-terminal region and is conserved between members of the same subgroup. This domain is necessary for hetero- but not homomultimeric assembly within a subfamily and prevents co-assembly between subfamilies. Recently, pore-forming subunits with two TMDs were also shown to co-assemble to form heteropolymers (Duprat et al. (1995) Biochem. Biophys. Res. Commun., 212, 657-663. This heteropolymerization seems necessary to give functional GIRKs. IRKs are active as homopolymers but also form heteropolymers.

New structural types of K⁺ channels were identified recently in both humans and yeast. These channels have two P domains in their functional subunit instead of only one (Ketchum et al. (1995) Nature, 376, 690-695; Lesage et al. (1996) J. Biol. Chem, 271, 4183-4187; Lesage et al. (1996) EMBO J., 15, 1004-1011; Reid et al. (1996) Receptors Channels 4, 51-62). The human channel called TWIK-1, has four TMDs. TWIK-1 is expressed widely in human tissues and is particularly abundant in the heart and the brain. TWIK-1 currents are time independent and inwardly rectifying. These properties suggest that TWIK-1 channels are involved in the control of the background K⁺ membrane conductance (Lesage et al. (1996) EMBO J., 15, 1004-1011).

### SUMMARY OF THE INVENTION

The present invention is based, at least in part, on the discovery of novel members of the TWIK (for Tandem of P domains in a Weak Inward rectifying K⁺ channel) family of potassium channels, referred to herein as TWIK-6 nucleic acid and protein molecules. The TWIK-6 molecules of the present invention are useful as targets for developing modulating agents to regulate a variety of cellular processes. Accordingly, in one aspect, this invention provides isolated nucleic acid molecules encoding TWIK proteins or biologically active portions thereof, as well as nucleic acid fragments suitable as primers or hybridization probes for the detection of TWIK-encoding nucleic acids.

In one embodiment, a TWIK-6 nucleic acid molecule of the invention is at least 50%, 55%, 60%, 65%, 70%, 73%, 74%, 75%, 80%, 85%, 90%, 95%, 96%, 97%, 98%, 99% or more identical to the nucleotide sequence (e.g., to the entire length of the nucleotide sequence) shown in SEQ ID NO:1 or 3 or SEQ ID NO:4 or 6 or the nucleotide sequence of the DNA insert of the plasmid deposited with ATCC as Accession Number ______, or a complement thereof.

In a preferred embodiment, the isolated nucleic acid molecule includes the nucleotide sequence shown SEQ ID NO:1 or 3, or SEQ ID NO:4 or 6, or a complement thereof. In another embodiment, the nucleic acid molecule includes SEQ ID NO:3 or 6 and nucleotides 1-102 of SEQ ID NO:1 or 4. In yet another embodiment, the nucleic acid molecule includes SEQ ID NO:3 or 6 and nucleotides 1306-1528 of SEQ ID NO:1 or 4. In another preferred embodiment, the nucleic acid molecule consists of the nucleotide sequence shown in SEQ ID NO:1 or 3 or SEQ ID NO:4 or 6. In another preferred embodiment, the nucleic acid molecule includes a fragment of at least 1051 nucleotides (e.g., 1051 contiguous nucleotides) of the nucleotide sequence of SEQ ID NO: 1 or 3 or SEQ ID NO:4 or 6, or a complement thereof.

In another embodiment, a TWIK-6 nucleic acid molecule includes a nucleotide sequence encoding a protein having an amino acid sequence sufficiently identical to the amino acid sequence of SEQ ID NO:2 or 5 or an amino acid sequence encoded by the DNA insert of the plasmid deposited with ATCC as Accession Number ______. In a preferred embodiment, a TWIK-6 nucleic acid molecule includes a nucleotide sequence encoding a protein having an amino acid sequence at least 50%, 55%, 60%, 65%, 67%, 68%, 70%, 75%, 80%, 85%, 90%, 95%, 96%, 97%, 98%, 99% or more identical to the entire length of the amino acid sequence of SEQ ID NO:2 or 5 or the amino acid sequence encoded by the DNA insert of the plasmid deposited with ATCC as Accession Number ______.

In another preferred embodiment, an isolated nucleic acid molecule encodes the amino acid sequence of human TWIK-6. In yet another preferred embodiment, the nucleic acid molecule includes a nucleotide sequence encoding a protein having the amino acid sequence of SEQ ID NO:2 or 5 or the amino acid sequence encoded by the DNA insert of the plasmid deposited with ATCC as Accession Number ______. In yet another preferred embodiment, the nucleic acid molecule is at least 1051 nucleotides in length. In a further preferred embodiment, the nucleic acid molecule is at least 1051 nucleotides in length and encodes a protein having a TWIK-6 activity (as described herein).

Another embodiment of the invention features nucleic acid molecules, preferably TWIK-6 nucleic acid molecules, which specifically detect TWIK-6 nucleic acid molecules relative to nucleic acid molecules encoding non-TWIK-6 proteins. For example, in one embodiment, such a nucleic acid molecule is at least 1050, 1051-1100, 1100-1150, 1150-1200, 1200-1250, 1250-1300, 1300-1350, 1350-1400, 1400-1450, 1450-1500, or more nucleotides in length and hybridizes under stringent conditions to a nucleic acid molecule comprising the nucleotide sequence shown in SEQ ID NO:1 or 4, the nucleotide sequence of the DNA insert of the plasmid deposited with ATCC as Accession Number ______, or a complement thereof.

In preferred embodiments, the nucleic acid molecules are at least 15 (e.g., 15 contiguous) nucleotides in length and hybridize under stringent conditions to nucleotides 1-156 of SEQ ID NO:1 or 4. In other preferred embodiments, the nucleic acid molecules comprise nucleotides 1-156 of SEQ ID NO:1 or 4.

In other preferred embodiments, the nucleic acid molecule encodes a naturally occurring allelic variant (e.g., an allelic variant as described in Example 1, where a single base change of A to G at nucleotide position 596 was identified) of a polypeptide comprising the amino acid sequence of SEQ ID NO:2 or an amino acid sequence encoded by the DNA insert of the plasmid deposited with ATCC as Accession Number ______, wherein the nucleic acid molecule hybridizes to a nucleic acid molecule comprising SEQ ID NO:1 or 3 or SEQ ID NO:4 or 6 under stringent conditions.

Another embodiment of the invention provides an isolated nucleic acid molecule which is antisense to a TWIK-6 nucleic acid molecule, e.g., the coding strand of a TWIK-6 nucleic acid molecule.

Another aspect of the invention provides a vector comprising a TWIK-6 nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably a TWIK-6 protein, by culturing in a suitable medium, a host cell, e.g., a mammalian host cell such as a non-human mammalian cell, of the invention containing a recombinant expression vector, such that the protein is produced.

Another aspect of this invention features isolated or recombinant TWIK-6 proteins and polypeptides. In one embodiment, an isolated TWIK-6 protein includes at least one transmembrane domain. In yet another embodiment, an isolated TWIK-6 protein includes a TWIK-related ion channel domain. In yet another embodiment, an isolated TWIK-6 protein includes a potassium channel protein domain. In yet another embodiment, an isolated TWIK-6 protein includes a P-loop domain. In yet another embodiment, an isolated TWIK-6 protein includes at least one transmembrane domain and one or more of the following domains: a TWIK-related ion channel domain, a P-loop domain, and a potassium channel protein domain. In yet another embodiment, an isolated TWIK-6 protein includes at least one transmembrane domain, a TWIK-related ion channel domain, a P-loop domain, and a potassium channel protein domain.

In a preferred embodiment, a TWIK-6 protein includes at least one transmembrane domain and has an amino acid sequence at least about 50%, 55%, 60%, 65%, 67%, 68%, 70%, 75%, 80%, 85%, 90%, 95%, 96%, 97%, 98%, 99% or more identical to the amino acid sequence of SEQ ID NO:2 or 5, or the amino acid sequence encoded by the DNA insert of the plasmid deposited with ATCC as Accession Number ______. In a further preferred embodiment, a TWIK-6 protein includes a TWIK-related ion channel domain and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:2 or 5, or the amino acid sequence encoded by the DNA insert of the plasmid deposited with ATCC as Accession Number ______. In a further preferred embodiment, a TWIK-6 protein includes a potassium channel protein domain and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:2 or 5, or the amino acid sequence encoded by the DNA insert of the plasmid deposited with ATCC as Accession Number ______. In a further preferred embodiment, a TWIK-6 protein includes at least one P-loop domain and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:2 or 5, or the amino acid sequence encoded by the DNA insert of the plasmid deposited with ATCC as Accession Number ______. In a further preferred embodiment, a TWIK-6 protein includes at least one transmembrane domain and one or more of a TWIK-related ion channel domain,, a P-loop domain, and/or a potassium channel protein domain, and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:2 or 5, or the amino acid sequence encoded by the DNA insert of the plasmid deposited with ATCC as Accession Number ______.

In another preferred embodiment, a TWIK-6 protein includes at least one transmembrane domain and has a TWIK-6 activity (as described herein).

In yet another preferred embodiment, a TWIK-6 protein includes at least one transmembrane domain and is encoded by a nucleic acid molecule having a nucleotide sequence which hybridizes under stringent hybridization conditions to a nucleic acid molecule comprising the nucleotide sequence of SEQ ID NO:1 or 3 or SEQ ID NO:4 or 6. In a further embodiment, a TWIK-6 protein includes a TWIK-related ion channel domain and is encoded by a nucleic acid molecule having a nucleotide sequence which hybridizes under stringent hybridization conditions to a nucleic acid molecule comprising the nucleotide sequence of SEQ ID NO:1 or 3 or 4 or 6. In a further embodiment, a TWIK-6 protein includes a P-loop domain and is encoded by a nucleic acid molecule having a nucleotide sequence which hybridizes under stringent hybridization conditions to a nucleic acid molecule comprising the nucleotide sequence of SEQ ID NO:1 or 3 or SEQ ID NO:4 or 6. In a further embodiment, a TWIK-6 protein includes a potassium channel protein domain and is encoded by a nucleic acid molecule having a nucleotide sequence which hybridizes under stringent hybridization conditions to a nucleic acid molecule comprising the nucleotide sequence of SEQ ID NO:1 or 3 or SEQ ID NO:4 or 6. In another embodiment, a TWIK-6 protein includes at least one transmembrane domain and one or more of a TWIK-related ion channel domain, a P-loop domain, and/or a potassium channel protein domain, and is encoded by a nucleic acid molecule having a nucleotide sequence which hybridizes under stringent hybridization conditions to a nucleic acid molecule comprising the nucleotide sequence of SEQ ID NO: 1 or 3 or SEQ ID NO:4 or 6.

In another embodiment, the invention features fragments of the protein having the amino acid sequence of SEQ ID NO:2 or 5, wherein the fragment comprises at least 19 amino acids (e.g., contiguous amino acids) of the amino acid sequence of SEQ ID NO:2 or 5, or an amino acid sequence encoded by the DNA insert of the plasmid deposited with the ATCC as Accession Number ______. In another embodiment, a TWIK-6 protein has the amino acid sequence of SEQ ID NO:2 or 5.

In another embodiment, the invention features a TWIK-6 protein which is encoded by a nucleic acid molecule consisting of a nucleotide sequence at least about 50%, 55%, 60%, 65%, 70%, 73%, 74%, 75%, 80%, 85%, 90%, 95%, 96%, 97%, 98%, 99% or more identical to a nucleotide sequence of SEQ ID NO:1 or 3, or SEQ ID NO:4 or 6, or a complement thereof. This invention further features a TWIK-6 protein, which is encoded by a nucleic acid molecule consisting of a nucleotide sequence which hybridizes under stringent hybridization conditions to a nucleic acid molecule comprising the nucleotide sequence of SEQ ID NO:1 or 3, or SEQ ID NO:4 or 6, or a complement thereof.

The proteins of the present invention or portions thereof, e.g., biologically active portions thereof, can be operatively linked to a non-TWIK-6 polypeptide (e.g., heterologous amino acid sequences) to form fusion proteins. The invention further features antibodies, such as monoclonal or polyclonal antibodies, that specifically bind proteins of the invention, preferably TWIK-6 proteins. In addition, the TWIK-6 proteins or biologically active portions thereof can be incorporated into pharmaceutical compositions, which optionally include pharmaceutically acceptable carriers.

In another aspect, the present invention provides a method for detecting the presence of a TWIK-6 nucleic acid molecule, protein, or polypeptide in a biological sample by contacting the biological sample with an agent capable of detecting a TWIK-6 nucleic acid molecule, protein, or polypeptide such that the presence of a TWIK-6 nucleic acid molecule, protein or polypeptide is detected in the biological sample.

In another aspect, the present invention provides a method for detecting the presence of TWIK-6 activity in a biological sample by contacting the biological sample with an agent capable of detecting an indicator of TWIK-6 activity such that the presence of TWIK-6 activity is detected in the biological sample.

In another aspect, the invention provides a method for modulating TWIK-6 activity comprising contacting a cell capable of expressing TWIK-6 with an agent that modulates TWIK-6 activity such that TWIK-6 activity in the cell is modulated. In one embodiment, the agent inhibits TWIK-6 activity. In another embodiment, the agent stimulates TWIK-6 activity. In one embodiment, the agent is an antibody that specifically binds to a TWIK-6 protein. In another embodiment, the agent modulates expression of TWIK-6 by modulating transcription of a TWIK-6 gene or translation of a TWIK-6 mRNA. In yet another embodiment, the agent is a nucleic acid molecule having a nucleotide sequence that is antisense to the coding strand of a TWIK-6 mRNA or a TWIK-6 gene.

In one embodiment, the methods of the present invention are used to treat a subject having a disorder characterized by aberrant or unwanted TWIK-6 protein or nucleic acid expression or activity by administering an agent which is a TWIK-6 modulator to the subject. In one embodiment, the TWIK-6 modulator is a TWIK-6 protein. In another embodiment the TWIK-6 modulator is a TWIK-6 nucleic acid molecule. In yet another embodiment, the TWIK-6 modulator is a peptide, peptidomimetic, or other small molecule. In a preferred embodiment, the disorder characterized by aberrant or unwanted TWIK-6 protein or nucleic acid expression is a CNS disorder, such as a cognitive or neurodegenerative disorder. In another preferred embodiment, the disorder characterized by aberrant or unwanted TWIK-6 protein or nucleic acid expression is a cardiovascular disorder. In another preferred embodiment, the disorder characterized by aberrant or unwanted TWIK-6 protein or nucleic acid expression is a muscular disorder. In another embodiment, the disorder characterized by aberrant or unwanted TWIK-6 activity is a cell proliferation, growth, differentiation, or migration disorder.

The present invention also provides diagnostic assays for identifying the presence or absence of a genetic alteration characterized by at least one of (i) aberrant modification or mutation of a gene encoding a TWIK-6 protein; (ii) mis-regulation of the gene; and (iii) aberrant post-translational modification of a TWIK-6 protein, wherein a wild-type form of the gene encodes a protein with a TWIK-6 activity.

In another aspect the invention provides methods for identifying a compound that binds to or modulates the activity of a TWIK-6 protein, by providing an indicator composition comprising a TWIK-6 protein having TWIK-6 activity, contacting the indicator composition with a test compound, and determining the effect of the test compound on TWIK-6 activity in the indicator composition to identify a compound that modulates the activity of a TWIK-6 protein.

Other features and advantages of the invention will be apparent from the following detailed description and claims.

### BRIEF DESCRIPTION OF THE DRAWINGS

FIGS. 1A-1B depict the cDNA sequence and predicted amino acid sequence of human TWIK-6 (A⁵⁹⁶). The nucleotide sequence corresponds to nucleic acids 1 to 1528 of SEQ ID NO:1. The amino acid sequence corresponds to amino acids 1 to 400 of SEQ ID NO:2. The coding region without the 3′ untranslated region of the human TWIK-6 gene is shown in SEQ ID NO:3.

FIGS. 2A-2B depict the cDNA sequence and predicted amino acid sequence of human TWIK-6 (G⁵⁹⁶) containing an A to G polymorphism at nucleotide 596. The nucleotide sequence corresponds to nucleic acids 1 to 1528 of SEQ ID NO:4. The amino acid sequence corresponds to amino acids 1 to 400 of SEQ ID NO:5. The coding region without the 3′ untranslated region of the human TWIK-6 gene is shown in SEQ ID NO: 6.

FIG. 3 depicts a structural, hydrophobicity, and antigenicity analysis of the human TWIK-6 protein.

FIGS. 4A-4B depict an NBLAST alignment of the human TWIK-6 nucleic acid sequence (SEQ ID NO:1) with the nucleic acid sequence of Homo sapiens TWIK-related acid-sensitive K+ channel (TASK) mRNA (Accession Number AF006823, SEQ ID NO:7) using a score of 100 and a wordlength of 12. The results show that the nucleotide sequence of human TWIK-6 is 72% identical to Accession Number AF006823 over nucleotides 200-1158 of SEQ ID NO: 1.

FIG. 5 depicts a BLASTX alignment of the human TWIK-6 predicted amino acid sequence (SEQ ID NO:2) with the amino acid sequence of Homo sapiens TWIK-related acid-sensitive K+ channel (Accession Number AF006823, SEQ ID NO:8) using a score of 100, a BLOSUM 62 matrix, and a wordlength of 3. The results show that the amino acid sequence of human TWIK-6 is 65% identical to that of Accession Number AF006823 over translated nucleotides 313-1086 of SEQ ID NO:2, 40% identical over translated nucleotides 1265 to 1354 of SEQ ID NO:2, and 33% identical over translated nucleotides 1253 to 1369 of SEQ ID NO:2.

FIG. 6 depicts an alignment of the human TWIK-6 amino acid sequence (SEQ ID NO:2, identified as 17831fl) with the amino acid sequences of TWIK-related acid-sensitive K+ channel from Homo sapiens (Accession Number AF006823, SEQ ID NO:8, identified as 2465542_TWIK_human) and TWIK-related acid-sensitive K+ channel from Rattus norvegicus (Accession Number AF031384, SEQ ID NO:9, identified as 2809391_TWIK_rat) using the CLUSTAL W (1.74) multiple sequence alignment program.

FIG. 7 depicts the results of a search which was performed against the MEMSAT database and which resulted in the identification of four “transmembrane domains” and two “Pore-loops” in the human TWIK-6 protein (SEQ ID NO:2 or SEQ ID NO:5). The human TWIK-6 amino acid sequence is also shown (SEQ ID NO:2)

FIG. 8 depicts the results of a search which was performed against the ProDom database and which resulted in the identification of a “potassium channel protein domain” (SEQ ID NOs:10, 11, and 12) and a TWIK-related ion channel domain (SEQ ID NO:13) in the human TWIK-6 protein (SEQ ID NO:2).

FIGS. 9A-B depict the cDNA sequence and predicted amino acid sequence of human TWIK-7. The nucleotide sequence corresponds to nucleic acids 1 to 1943 of SEQ ID NO:14. The amino acid sequence corresponds to amino acids 1 to 431 of SEQ ID NO:15. The coding region without the 3′ untranslated region of the human TWIK-7 gene is shown in SEQ ID NO:16.

FIG. 10 depicts a structural, hydrophobicity, and antigenicity analysis of the human TWIK-7 protein.

FIGS. 11A-11C depict an alignment of the human TWIK-7 nucleic acid sequence (SEQ ID NO:14) with the nucleic acid sequence of Homo sapiens TWIK-related acid sensitive K+ channel (TASK) mRNA (Accession Number AF006823; SEQ ID NO:17), using the GAP program in the GCG software package (nwsgapdna matrix), a gap weight of 50, and a length weight of 3.

FIGS. 12A-12B depict an alignment of the human TWIK-7 amino acid sequence (SEQ ID NO:15) with the amino acid sequence of Homo sapiens TWIK related acid sensitive K+ channel (Accession Number AAC51777; SEQ ID NO:18), using the GAP program in the GCG software package (Blosum 62 matrix), a gap weight of 12, and a length weight of 4.

FIG. 13 depicts the results of a search which was performed against the MEMSAT database and which resulted in the identification of four “transmembrane domains” and two “Pore-loop domains” in the human TWIK-7 protein (SEQ ID NO:15).

FIG. 14 depicts the results of a search which was performed against the ProDom database and which resulted in the identification of a “potassium channel protein domain” (SEQ ID NO:19) in the human TWIK-7 protein (SEQ ID NO:15).

FIG. 15 depicts an alignment of the human TWIK-7 amino acid sequence (SEQ ID NO:15) with the amino acid sequences of TWIK-1 from Mus musculus (“mTWIK1”; Accession No. AAC16973; SEQ ID NO:20), and of TWIK-related acid-sensitive K+ channel (“hTASK”; Accession No. AF006823; SEQ ID NO:17) from Homo sapiens, using the CLUSTAL W (1.74) multiple sequence alignment program.

FIGS. 16A-G depict the cDNA sequence and predicted amino acid sequence of human IC23927. The nucleotide sequence corresponds to nucleic acids 1 to 5269 of SEQ ID NO:21. The amino acid sequence corresponds to amino acids 1 to 816 of SEQ ID NO:22. The stop codon is indicted by an asterisk. The coding region without the 3′ untranslated region of the human IC23927 gene is shown in SEQ ID NO:23.

FIG. 17 depicts a structural, hydrophobicity, and antigenicity analysis of the human IC23927 protein.

FIGS. 18A-C depict an alignment of the amino acid sequence of human IC23927 (SEQ ID NO:22) with the amino acid sequence of GenBank™ Accession No. BAA76556, corresponding to a Rattus norvegicus voltage-gated Ca channel (SEQ ID NO:24) and GenBank™ Accession No. AAD15312 corresponding to an Arabidopsis thaliana putative calcium channel (SEQ ID NO:25). The initial pairwise alignment step was performed using a Lipman Pearson algorithm with a K-tuple of 1, a GAP penalty of 3, a window of 5, and diagonals saved set to 5. The multiple alignment step was performed using the Clustal algorithm with a PAM 250 residue weight Table, a GAP penalty of 10, and a GAP length penalty of 10.

FIG. 19 depicts the cDNA sequence and predicted amino acid sequence of human TWIK-8. The nucleotide sequence corresponds to nucleic acids 1 to 1408 of SEQ ID NO:28. The amino acid sequence corresponds to amino acids 1 to 419 of SEQ ID NO:29. The coding region without the 3′ untranslated region of the human TWIK-8 gene is shown in SEQ ID NO:30.

FIG. 20 depicts the results of an analysis of the human TWIK-8 amino acid sequence (SEQ ID NO:29) by the Signal P program (Henrik et al. (1997) Protein Eng. 10:1-6), indicating the presence of a signal peptide at about residues 1-46 of the native molecule.

FIG. 21 depicts the results of a search which was performed against the MEMSAT database and which resulted in the identification of six transmembrane domains in the native human TWIK-8 protein (SEQ ID NO:29), and five transmembrane domains in the mature form of the human TWIK-8 protein.

FIG. 22 depicts the results of a search performed against the HMM database which identified the presence of a “seven-transmembrane receptor domain” and a “cyclic nucleotide-gated channel domain” in the amino acid sequence of human TWIK-8 (SEQ ID NO:29).

FIGS. 23A-H depict the results of a search performed against the ProDom database which identified the presence of a “TRAAK potassium channel domain”, a “potassium channel protein domain”, a “voltage-gated potassium channel domain”, an “outward-rectifier TOK1 potassium channel domain”, and a “potassium channel subunit domain” in the amino acid sequence of human TWIK-8 (SEQ ID NO:29).

FIGS. 24A-24C depict the cDNA sequence and predicted amino acid sequence of human IC47611. The nucleotide sequence corresponds to nucleic acids 1 to 4037 of SEQ ID NO:31. The amino acid sequence corresponds to amino acids 1 to 453 of SEQ ID NO:32. The coding region without the 3′ untranslated region of the human IC47611 gene is shown in SEQ ID NO:33.

FIG. 25 depicts a hydrophobicity plot of the amino acid sequence of human IC47611 (SEQ ID NO:32).

FIG. 26 depicts the results of a search which was performed against the MEMSAT database and which resulted in the identification of two “transmembrane domains” in the human IC47611 protein (SEQ ID NO:32).

FIG. 27 depicts the results of a search which was performed against the HMM database and which resulted in the identification of an “inward-rectifier potassium channel (IRK) domain” in the human IC47611 protein (SEQ ID NO:32).

FIG. 28 depicts the results of a search which was performed against the ProDom database and which resulted in the identification of an “inward-rectifier potassium channel (IRK) domain” and a “inward-rectifier potassium channel (IRK)-related domain” in the human IC47611 protein (SEQ ID NO:32).

FIGS. 29A-29C depict the cDNA sequence and predicted amino acid sequence of human IC47615. The nucleotide sequence corresponds to nucleic acids 1 to 4003 of SEQ ID NO:34. The amino acid sequence corresponds to amino acids 1 to 305 of SEQ ID NO:35. The coding region without the 3′ untranslated region of the human IC47615 gene is shown in SEQ ID NO:36.

FIG. 30 depicts a hydrophobicity plot of the amino acid sequence of human IC47615 (SEQ ID NO:35).

FIG. 31 depicts the results of a search which was performed against the MEMSAT database and which resulted in the identification of one “transmembrane domain” in the human IC47615 protein (SEQ ID NO:35).

FIG. 32 depicts the cDNA sequence and predicted amino acid sequence of HNMDA-1. The nucleotide sequence corresponds to nucleic acids 1 to 4197 of SEQ ID NO:37. The amino acid sequence corresponds to amino acids 1 to 1115 of SEQ ID NO:38. The coding region without the 3′ untranslated region of the HNMDA-1 gene is shown in SEQ ID NO:39.

FIG. 33 depicts a structural, hydrophobicity, and antigenicity analysis of the HNMDA-1 polypeptide.

FIG. 34 depicts the results of a search which was performed against the HMM database in PFAM and which resulted in the identification of one “ligand-gated ion channel family domain” in the HNMDA-1 polypeptide (SEQ ID NO:38).

FIGS. 35A-B depict the results of a search which was performed against the HMM database in SMART and which resulted in the identification of one “glutamate-gated ion channel family domain” in the HNMDA-1 polypeptide (SEQ ID NO:38).

FIG. 36 depicts the results of a search which was performed against the MEMSAT database and which resulted in the identification of four “transmembrane domains” and one “signal peptide” in the HNMDA-1 polypeptide (SEQ ID NO:38).

FIG. 37 depicts an alignment of the HNMDA-1 amino acid sequence (SEQ ID NO:38) with the amino acid sequences of rat NMDA-L (Accession No. 1050330) using the CLUSTAL W (1.74) alignment program.

FIG. 38 depicts the nucleotide sequence of the human TWIK-9 cDNA and the corresponding amino acid sequence. The nucleotide sequence corresponds to nucleic acids 1 to 1262 of SEQ ID NO:40. The amino acid sequence corresponds to amino acids 1-374 of SEQ ID NO:41. The coding region without the 5′ or 3′ untranslated regions of the human TWIK-9 gene is shown in SEQ ID NO:42.

FIG. 39 depicts a structural, hydrophobicity, and antigenicity analysis of the human TWIK-9 protein.

FIGS. 40A-40B depicts the results of a search which was performed against the Washington University HMM database and which resulted in the identification of the protein of SEQ ID NO:41 as a TWIK channel family member.

FIGS. 41A-D depict the cDNA sequence and predicted amino acid sequence of human α₂δ-4. The nucleotide sequence corresponds to nucleic acids 1 to 5489 of SEQ ID NO:43. The amino acid sequence corresponds to amino acids 1 to 116 of SEQ ID NO:44. The coding region without the 5′ and 3′ untranslated regions of the human α₂δ-4 gene is shown in SEQ ID NO:45.

FIG. 42 depicts a structural, hydrophobicity, and antigenicity analysis of the human α₂δ-4 polypeptide (SEQ ID NO:44).

FIGS. 43A-43B depicts an alignment of the human α₂δ-4 amino acid sequence (SEQ ID NO:44) with the amino acid sequence of the human dihydropyridine-sensitive L-type calcium channel α₂δ subunit protein CIC2 (SwissProt Accession No. P54289), using the CLUSTAL W (1.74) alignment program.

FIGS. 44A-44D depict the cDNA sequence and predicted amino acid sequence of human 8099. The nucleotide sequence corresponds to nucleic acids 1 to 2725 of SEQ ID NO:46. The amino acid sequence corresponds to amino acids 1 to 617 of SEQ ID NO:47. The coding region without the 5′ and 3′ untranslated regions of the human 8099 gene is shown in SEQ ID NO:48.

FIG. 45 depicts a structural, hydrophobicity, and antigenicity analysis of the human 8099 polypeptide (SEQ ID NO:47).

FIGS. 46A-46G depict the results of a search which was performed against the HMM database in PFAM.

FIGS. 47A-B depicts an alignment of the human 8099 amino acid sequence (SEQ ID NO:47) with the amino acid sequence of the E. coli galactose-proton symporter GALP using the CLUSTAL W (1.74) alignment program (having GenBank Accession No. P37021, set forth as SEQ ID NO:73).

FIGS. 48A-B depict an alignment of the human 8099 amino acid sequence (SEQ ID NO:47) with the amino acid sequence of the E. coli arabinose-proton symporter ARAE using the CLUSTAL W (1.74) alignment program (having GenBank Accession No. P09830, set forth as SEQ ID NO:74).

FIGS. 49A-C depict an alignment of the human 8099 amino acid sequence (SEQ ID NO:47) with the amino acid sequence of E. coli GALP and ARAE using the CLUSTAL W (1.74) alignment program (having GenBank Accession Nos. P37021 and P09830, respectively, set forth as SEQ ID NOs:73 and 74, respectively).

FIGS. 50A-B depict an alignment of the human 8099 amino acid sequence (SEQ ID NO:47) with the amino acid sequence of the H. sapiens facilitative glucose transporter GLUT8 using the CLUSTAL W (1.74) alignment program (having GenBank Accession No. Y02168, set forth as SEQ ID NO:75).

FIGS. 51A-51D depict the cDNA sequence and predicted amino acid sequence of human 46455. The nucleotide sequence corresponds to nucleic acids 1 to 2230 of SEQ ID NO:49. The amino acid sequence corresponds to amino acids 1 to 528 of SEQ ID NO:50. The coding region without the 5′ and 3′ untranslated regions of the human 46455 gene is shown in SEQ ID NO:51.

FIG. 52 depicts a structural, hydrophobicity, and antigenicity analysis of the human 46455 polypeptide (SEQ ID NO:50).

FIGS. 53A-53G depict the results of a search which was performed against the HMM database in PFAM.

FIGS. 54A-B depict an alignment of the human 46455 amino acid sequence (SEQ ID NO:50) with the amino acid sequence of C. elegans Z92825 using the CLUSTAL W (1.74) alignment program (having GenBank Accession No. Z92825, set forth as SEQ ID NO:76).

FIGS. 55A-55H depict the nucleotide sequence of the human 54414 cDNA and the corresponding amino acid sequence. The nucleotide sequence corresponds to nucleic acids 1 to 4632 of SEQ ID NO:52. The amino acid sequence corresponds to amino acids 1 to 1118 of SEQ ID NO:53. The coding region without the 5′ or 3′ untranslated regions of the human 54414 gene is shown in SEQ ID NO:54.

FIG. 56 depicts a structural, hydrophobicity, and antigenicity analysis of the human 54414 polypeptide (SEQ ID NO:53). The locations of the 6 transmembrane domains, as well as the pore domain (P), are indicated.

FIG. 57B depict the results of a search in the HMM database, using the amino acid sequence of human 54414.

FIGS. 58A-58D depict a Clustal W (1.74) multiple sequence alignment of the human 54414 amino acid sequence (54414.prot; SEQ ID NO:53) and the amino acid sequence of the Rattus norvegicus Slack potassium channel subunit (AF089730; SEQ ID NO:77; GenBank Accession No. AAC83350). Amino acid identities are indicated by stars. The six transmembrane domains (TM1, TM2, etc.) are boxed. The pore domain, which contains the potassium channel signature sequence motif, is also boxed.

FIGS. 59A-59D depict the nucleotide sequence of the human 53763 cDNA and the corresponding amino acid sequence. The nucleotide sequence corresponds to nucleic acids 1 to 2847 of SEQ ID NO:55. The amino acid sequence corresponds to amino acids 1 to 638 of SEQ ID NO:56. The coding region without the 5′ or 3′ untranslated regions of the human 53763 gene is shown in SEQ ID NO:57.

FIG. 60 depicts a structural, hydrophobicity, and antigenicity analysis of the human 53763 polypeptide (SEQ ID NO:56). The locations of the 6 transmembrane domains, as well as the pore domain (P), are indicated.

FIGS. 61A-61E depict the results of a search in the HMM database, using the amino acid sequence of human 53763.

FIGS. 62A-B depict a Clustal W (1.74) sequence alignment of the human 53763 amino acid sequence (Fbh53763pat; SEQ ID NO:56) and the amino acid sequence of the Rattus norvegicus voltage-gated potassium channel protein KV3.2 (KSHIIIA) (ratCIKE; SEQ ID NO:78; GenBank Accession No. P22462). Amino acid identities are indicated by stars. The six transmembrane domains (TM1, TM2, etc.) are boxed. The pore domain, which contains the potassium channel signature sequence motif, is also boxed. Plus signs (+) at every third position of the fourth transmembrane domain (TM4), indicate the positively charged residues of the voltage sensor.

FIGS. 63A-63H depict the cDNA sequence and predicted amino acid sequence of human 67076. The nucleotide sequence corresponds to nucleic acids 1 to 6582 of SEQ ID NO:58. The amino acid sequence corresponds to amino acids 1 to 1129 of SEQ ID NO:59. The coding region without the 5′ and 3′ untranslated regions of the human 67076 gene is shown in SEQ ID NO:60.

FIG. 64 depicts a structural, hydrophobicity, and antigenicity analysis of the human 67076 polypeptide (SEQ ID NO:59).

FIGS. 65A-C depict the results of a search in the HMM database, using the amino acid sequence of human 67076.

FIGS. 66A-D depict a Clustal W (1.74) alignment of the human 67076 amino acid sequence (“Fbh67076FL”; SEQ ID NO:59) with the amino acid sequence of mouse Potential Phospholipid-Transporting ATPase IH (mouseAT1H) (GenBank Accession No. P98197) (SEQ ID NO:79). The transmembrane domains (“TM1”, “TM2”, etc.), E1-E2 ATPases phosphorylation site (“phosphorylation site”), and phospholipid transporter specific amino acid residues (“phospholipid transport”) are boxed.

FIGS. 67A-67I depict the cDNA sequence and predicted amino acid sequence of human 67102. The nucleotide sequence corresponds to nucleic acids 1 to 6074 of SEQ ID NO:61. The amino acid sequence corresponds to amino acids 1 to 1426 of SEQ ID NO:62. The coding region without the 5′ and 3′ untranslated regions of the human 67102 gene is shown in SEQ ID NO:63.

FIG. 68 depicts a structural, hydrophobicity, and antigenicity analysis of the human 67102 polypeptide (SEQ ID NO:62).

FIGS. 69A-69E depict the results of a search in the HMM database, using the amino acid sequence of human 67102.

FIGS. 70A-70E depict a Clustal W (1.74) alignment of the human 67102 amino acid sequence (“Fbh67102FL”; SEQ ID NO:62) with the amino acid sequence of mouse Potential Phospholipid-Transporting ATPase VA (mouseAT5A) (GenBank Accession No. O54827) (SEQ ID NO:80). The transmembrane domains (“TM1”, “TM2”, etc.), E1-E2 ATPases phosphorylation site (“phosphorylation site”), and phospholipid transporter specific amino acid residues (“phospholipid transport”) are boxed.

FIGS. 71A-71J depict the cDNA sequence and predicted amino acid sequence of human 44181. The nucleotide sequence corresponds to nucleic acids 1 to 7221 of SEQ ID NO:64. The amino acid sequence corresponds to amino acids 1 to 1177 of SEQ ID NO:65. The coding region without the 5′ and 3′ untranslated regions of the human 44181 gene is shown in SEQ ID NO:66.

FIG. 72 depicts a structural, hydrophobicity, and antigenicity analysis of the human 44181 polypeptide (SEQ ID NO:65).

FIGS. 73A-73D depict the results of a search in the HMM database, using the amino acid sequence of human 44181.

FIGS. 74A-74E depict a Clustal W (1.74) multiple sequence alignment of the human 44181 amino acid sequence (“Fbh44181”; SEQ ID NO:65) with the amino acid sequence of mouse Potential Phospholipid-Transporting ATPase IH (mouseAT1H) (GenBank Accession No. P98197) (SEQ ID NO:79) and 67076 (“Fbh67076FL”; SEQ ID NO:59). The transmembrane domains (“TM1”, “TM2”, etc.), E1-E2 ATPases phosphorylation site (“phosphorylation site”), and phospholipid transporter specific amino acid residues (“phospholipid transport”) are boxed.

FIGS. 75A-75G depict the cDNA sequence and predicted amino acid sequence of human 67084FL. The nucleotide sequence corresponds to nucleic acids 1 to 4198 of SEQ ID NO:67. The amino acid sequence corresponds to amino acids 1 to 1084 of SEQ ID NO:68. The coding region without the 5′ and 3′ untranslated regions of the human 67084FL gene is shown in SEQ ID NO:69.

FIG. 76 depicts a structural, hydrophobicity, and antigenicity analysis of the human 67084FL polypeptide (SEQ ID NO:68).

FIGS. 77A-77C depict the results of a search in the HMM database, using the amino acid sequence of human 67084FL.

FIGS. 78A-78C depict a Clustal W (1.74) alignment of the human 67084FL amino acid sequence (“Fbh67084FL”; SEQ ID NO:68) with the amino acid sequence of mouse Potential Phospholipid-Transporting ATPase IIV (mouseAT2B) (GenBank Accession No.:P98195) (SEQ ID NO:81). The transmembrane domains (“TM1”, “TM2”, etc.), E1-E2 ATPases phosphorylation site (“phosphorylation site”), and phospholipid transporter specific amino acid residues (“phospholipid transport”) are boxed.

FIGS. 79A-79G depict the cDNA sequence and predicted amino acid sequence of human 67084alt. The nucleotide sequence corresponds to nucleic acids 1 to 4231 of SEQ ID NO:70. The amino acid sequence corresponds to amino acids 1 to 1095 of SEQ ID NO:71. The coding region without the 5′ and 3′ untranslated regions of the human 67084alt gene is shown in SEQ ID NO:72.

FIG. 80 depicts a structural, hydrophobicity, and antigenicity analysis of the human 67084alt polypeptide (SEQ ID NO:71).

FIGS. 81A-81C depict the results of a search in the HMM database, using the amino acid sequence of human 67084.

FIGS. 82A-82C depict a Clustal W (1.74) alignment of the human 67084alt amino acid sequence (“Fbh67084alt”; SEQ ID NO:71) with the amino acid sequence of mouse Potential Phospholipid-Transporting ATPase IIV (mouseAT2B) (GenBank Accession No.:P98195) (SEQ ID NO:81). The transmembrane domains (“TM1”, “TM2”, etc.), E1-E2 ATPases phosphorylation site (“phosphorylation site”), and phospholipid transporter specific amino acid residues (“phospholipid transport”) are boxed.

### DETAILED DESCRIPTION OF THE INVENTION

The present invention is based, at least in part, on the discovery of novel molecules, referred to herein as TWIK-6 nucleic acid and protein molecules, which are novel members of the TWIK (for Tandem of P domains in a Weak Inward rectifying K⁺ channel) family of potassium channels. These novel molecules are capable of, for example, modulating a potassium channel mediated activity in a cell, e.g., a neuronal cell, a muscle cell (e.g., a cardiac muscle), or a thymus cell.

As used herein, a “potassium channel” includes a protein or polypeptide which is involved in receiving, conducting, and transmitting signals in an electrically excitable cell, e.g., a neuronal cell or a muscle cell. Potassium channels are potassium ion selective, and can determine membrane excitability (the ability of, for example, a neuron to respond to a stimulus and convert it into an impulse). Potassium channels can also influence the resting potential of membranes, wave forms and frequencies of action potentials, and thresholds of excitation. Potassium channels are typically expressed in electrically excitable cells, e.g., neurons, muscle, endocrine, and egg cells, and may form heteromultimeric structures, e.g., composed of pore-forming α and cytoplasmic β subunits. Potassium channels may also be found in nonexcitable cells (e.g., thymus cells), where they may play a role in, e.g., signal transduction. Examples of potassium channels include: (1) the voltage-gated potassium channels, (2) the ligand-gated potassium channels, e.g., neurotransmitter-gated potassium channels, and (3) cyclic-nucleotide-gated potassium channels. Voltage-gated and ligand-gated potassium channels are expressed in the brain, e.g., in brainstem monoaminergic and forebrain cholinergic neurons, where they are involved in the release of neurotransmitters, or in the dendrites of hippocampal and neocortical pyramidal cells, where they are involved in the processes of learning and memory formation. For a detailed description of potassium channels, see Kandel E. R.. et al., Principles of Neural Science, second edition, (Elsevier Science Publishing Co., Inc., N.Y. (1985)), the contents of which are incorporated herein by reference. As the TWIK proteins of the present invention may modulate potassium channel mediated activities, they may be useful for developing novel diagnostic and therapeutic agents for potassium channel associated disorders.

As used herein, a “potassium channel associated disorder” includes a disorder, disease or condition which is characterized by a misregulation of a potassium channel mediated activity. Potassium channel associated disorders can detrimentally affect conveyance of sensory impulses from the periphery to the brain and/or conductance of motor impulses from the brain to the periphery; integration of reflexes; interpretation of sensory impulses; cellular proliferation, growth, differentiation, or migration, and emotional, intellectual (e.g., learning and memory), or motor processes. Examples of potassium channel associated disorders include CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further examples of potassium channel associated disorders include cardiac-related disorders. Cardiovascular system disorders in which the TWIK-6 molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia. TWIK-6-mediated or related disorders also include disorders of the musculoskeletal system such as paralysis and muscle weakness, e.g., ataxia, myotonia, and myokymia.

Potassium channel disorders also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The TWIK-6 molecules of the present invention are involved in signal transduction mechanisms, which are known to be involved in cellular growth, differentiation, and migration processes. Thus, the TWIK-6 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementia; hepatic disorders; cardiovascular disorders; and hematopoietic and/or myeloproliferative disorders.

TWIK-6-associated or related disorders also include disorders of tissues in which TWIK-6 protein is expressed, e.g., thymus, salivary gland, primary cultured osteoblasts, and ovarian epithelium tumor cells. Such disorders include, for example, proliferative disorders. As used herein, a “potassium channel mediated activity” includes an activity which involves a potassium channel, e.g., a potassium channel in a neuronal cell, a muscle cell, or a thymus cell associated with receiving, conducting, and transmitting signals in, for example, the nervous system. Potassium channel mediated activities include release of neurotransmitters, e.g., dopamine or norepinephrine, from cells, e.g., neuronal cells; modulation of resting potential of membranes, wave forms and frequencies of action potentials, and thresholds of excitation; participation in signal transduction pathways, and modulation of processes such as integration of sub-threshold synaptic responses and the conductance of back-propagating action potentials in, for example, neuronal cells or muscle cells.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., monkey proteins. Members of a family may also have common functional characteristics.

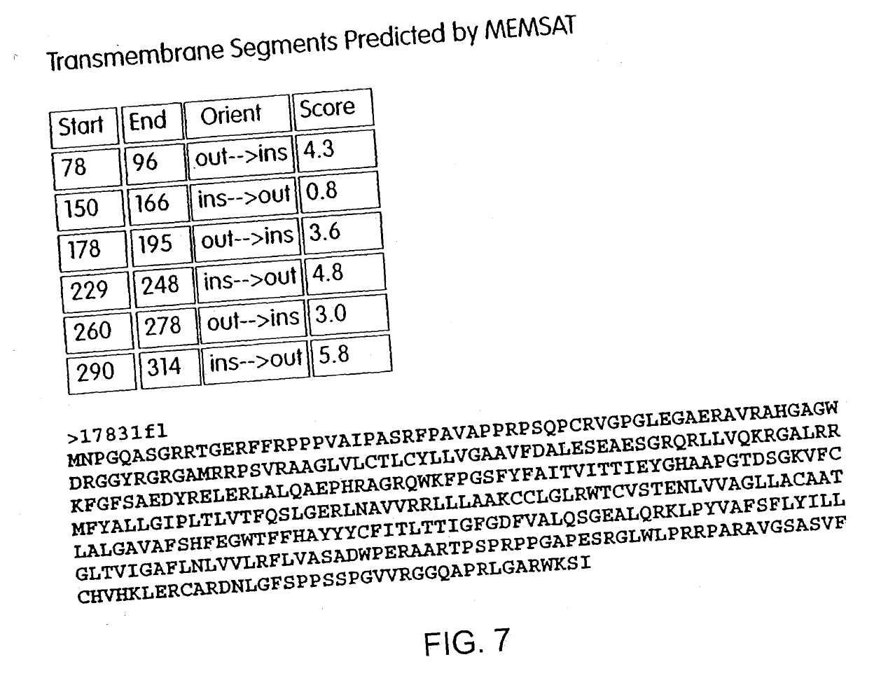

For example, the family of TWIK-6 proteins comprises at least one “transmembrane domain” and preferably four transmembrane domains. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al., (1996) Annual Rev. Neurosci. 19: 235-263, the contents of which are incorporated herein by reference. Amino acid residues 78-96, 178-195, 229-248, and 290-314 of the native TWIK-6 protein are predicted to comprise transmembrane domains (see FIG. 7). Accordingly, TWIK-6 proteins having at least 50-60% homology, preferably about 60-70%, more preferably about 70-80%, or about 80-90% homology with a transmembrane domain of human TWIK-6 are within the scope of the invention.

In another embodiment, a TWIK-6 molecule of the present invention is identified based on the presence of a Pore loop or P-loop. As used herein, the term “Pore loop” or “P-loop” includes amino acid sequence of about 15-45 amino acid residues in length, preferably about 15-35 amino acid residues in length, and most preferably about 15-25 amino acid residues in length, which is hydrophobic and which is involved in lining the potassium channel pore. A P-loop is typically found between transmembrane domains of potassium channels and is believed to be a major determinant of ion selectivity in potassium channels. Preferably, P-loops contain a G-[HYDROPHOBIC AMINO ACID]-G sequence, e.g., a GYG, GLG, or GFG sequence. P-loops are described in, for example, Wamike et al. (1991) Science 252:1560-1562; Zagotta W. N. et al., (1996) Annual Rev. Neurosci. 19:235-63 (Pongs, O. (1993) J. Membr. Biol., 136, 1-8; Heginbotham et al. (1994) Biophys. J. 66,1061-1067; Mackinnon, R. (1995) Neuron, and 14, 889-892; Pascual et al., (1995) Neuron., 14, 1055-1063), the contents of which are incorporated herein by reference. Amino acid residues 150-166 and 260-278 of SEQ ID NO:2 comprise P-loop domains, as do amino acid residues 150-166 and 260-278 of SEQ ID NO:5. The human TWIK-6 gene contains a nucleotide polymorphism at nucleotide 596 (A to G) which results in an amino acid alteration at amino acid position 165 (E to G). The 165E form of the protein is set forth in SEQ ID NO:2, and lacks the “GYG” P-loop motif which the 165G form of the protein (SEQ ID NO:5) possesses at residues 165-167. Thus, the 165E form of the human TWIK-6 protein (SEQ ID NO:2) has only one “G-[hydrophobic]-G” motif in its two P-loop domains, while the 165G form of the human TWIK-6 protein (SEQ ID NO:5) has two “G-[hydrophobic]-G” motifs in its two P-loop domains.

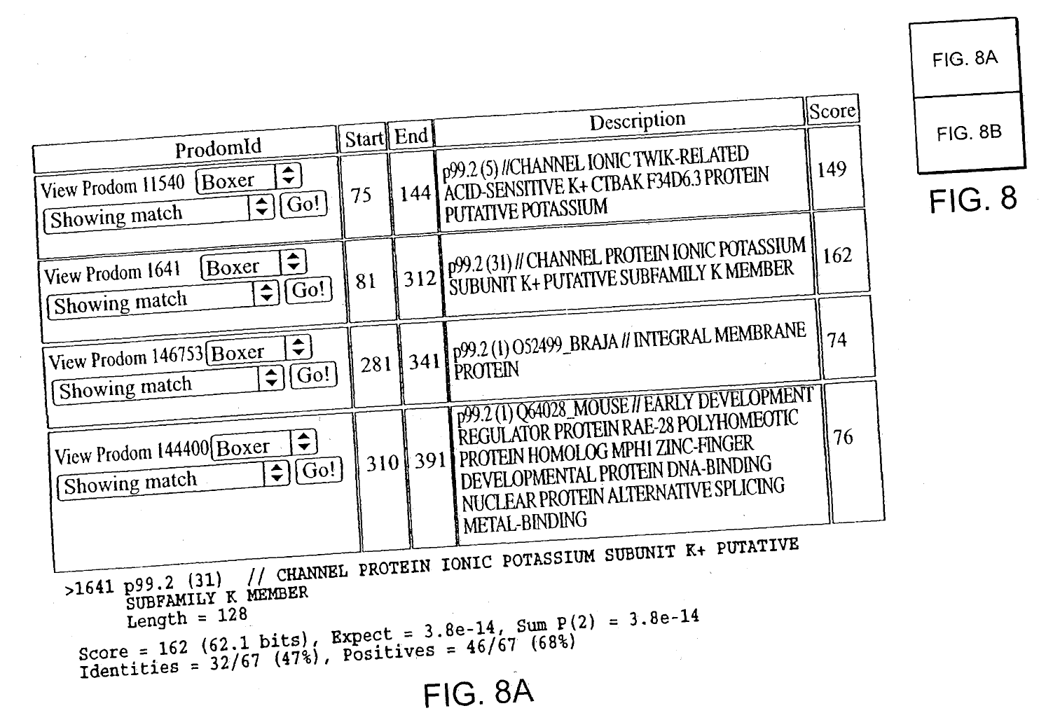

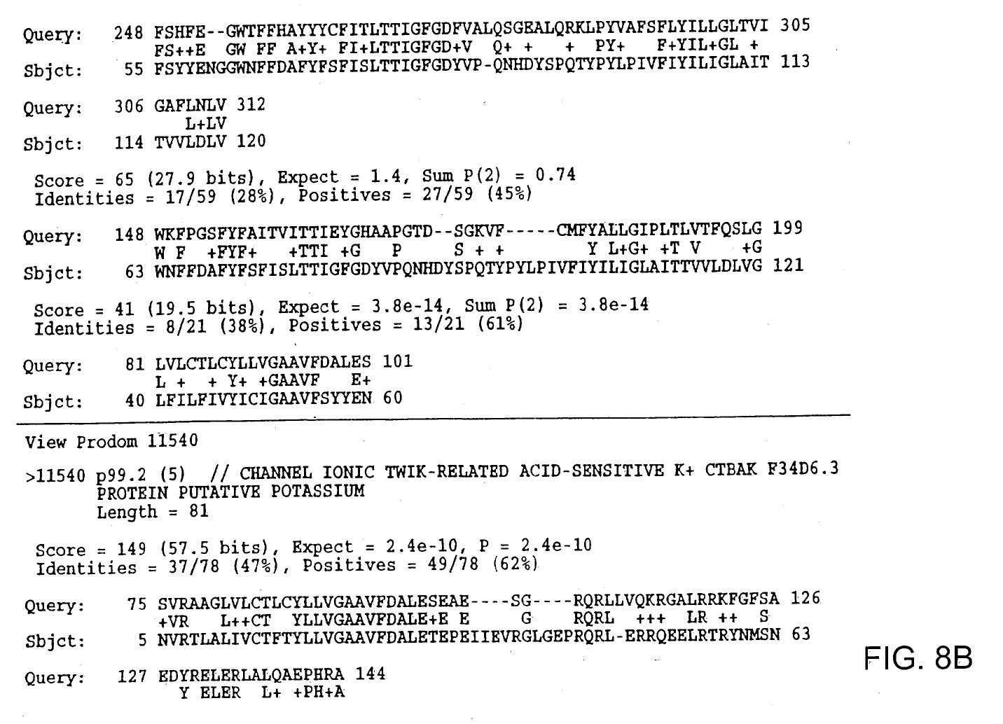

In another embodiment, a TWIK-6 molecule of the present invention is identified based on the presence of a “potassium channel protein domain” in the protein or corresponding nucleic acid molecule. As used herein, the term “potassium channel protein domain” includes a protein domain having an amino acid sequence of about 150-250 amino acid residues and having a bit score for the alignment of the sequence to the TWIK-related ion channel domain of at least 162. Preferably, a TWIK-related ion channel domain includes at least about 200-240, or more preferably about 232 amino acid residues, and has a bit score for the alignment of the sequence to the TWIK-related ion channel domain of at least 20, 25, 30, 35, 40, 45, 50, 55, 60, 65, 70, 75, 80, 85, 90, 95, 100, 105, 110, 115, 120, 125, 130, 135, 140, 145, 150, 155, 160 or higher. The potassium channel protein domain has been assigned ProDom entry 1641. To identify the presence of a potassium channel protein domain in a TWIK-6 protein, and make the determination that a protein of interest has a particular profile, the amino acid sequence of the protein is searched against a database of known protein domains (e.g., the ProDom database) using the default parameters (available at http://www.toulouse.inra.fr/prodom.html). A search was performed against the ProDom database resulting in the identification of a potassium channel protein domain in the amino acid sequence of human TWIK-6 (SEQ ID NO:2 or 5) at about residues 81-312 of SEQ ID NO: 2 or 5. The results of the search are set forth in FIG. 8.

In another embodiment, a TWIK-6 molecule of the present invention is identified based on the presence of a “TWIK-related ion channel domain” in the protein or corresponding nucleic acid molecule. As used herein, the term “TWIK-related ion channel domain” includes a protein domain having an amino acid sequence of about 25-125 amino acid residues and having a bit score for the alignment of the sequence to the TWIK-related ion channel domain of at least 149. Preferably, a TWIK-related ion channel domain includes at least about 50-100, or more preferably about 70 amino acid residues, and has a bit score for the alignment of the sequence to the TWIK-related ion channel domain of at least 20, 25, 30, 35, 40, 45, 50, 55, 60, 65, 70, 75, 80, 85, 90, 95, 100, 105, 110, 115, 120, 125, 130, 135, 140, 145 or higher. The TWIK-related ion channel domain has been assigned ProDom entry 11540. To identify the presence of a TWIK-related ion channel domain in a TWIK-6 protein, and make the determination that a protein of interest has a particular profile, the amino acid sequence of the protein is searched against a database of known protein domains (e.g., the ProDom database) using the default parameters (available at http://www.toulouse.inra.fr/prodom.html). A search was performed against the ProDom database resulting in the identification of a TWIK-related ion channel domain in the amino acid sequence of human TWIK-6 (SEQ ID NO: 2 or 5) at about residues 75-144 of SEQ ID NO: 2 or SEQ ID NO:5. The results of the search are set forth in FIG. 8.

In a preferred embodiment, the TWIK-6 molecules of the invention include at least one transmembrane domain, at least one P-loop, at least one TWIK-related ion channel domain, and at least one potassium channel protein domain.

Isolated proteins of the present invention, preferably TWIK-6 proteins, have an amino acid sequence sufficiently identical to the amino acid sequence of SEQ ID NO:2 or 5 or are encoded by a nucleotide sequence sufficiently identical to SEQ ID NO:1 or 3 or SEQ ID NO:4 or 6. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, an “TWIK-6 activity”, “biological activity of TWIK-6” or “functional activity of TWIK-6”, refers to an activity exerted by a TWIK-6 protein, polypeptide or nucleic acid molecule on a TWIK-6 responsive cell or tissue, or on a TWIK-6 protein substrate, as determined in vivo, or in vitro, according to standard techniques. In one embodiment, a TWIK-6 activity is a direct activity, such as an association with a TWIK-6-target molecule. As used herein, a “target molecule” or “binding partner” is a molecule with which a TWIK-6 protein binds or interacts in nature, such that TWIK-6-mediated function is achieved. A TWIK-6 target molecule can be a non-TWIK-6 molecule or a TWIK-6 protein or polypeptide of the present invention. In an exemplary embodiment, a TWIK-6 target molecule is a TWIK-6 ligand, e.g., a potassium channel pore-forming subunit or a potassium channel ligand. Alternatively, a TWIK-6 activity is an indirect activity, such as a cellular signaling activity mediated by interaction of the TWIK-6 protein with a TWIK-6 ligand. The biological activities of TWIK-6 are described herein. For example, the TWIK-6 proteins of the present invention can have one or more of the following activities: (1) interacting with a non-TWIK protein molecule; (2) activating a TWIK-dependent signal transduction pathway; (3) modulating the release of neurotransmitters; (4) modulating membrane excitability; (5) influencing the resting potential of membranes, wave forms and frequencies of action potentials, and thresholds of excitation, and (6) modulating processes which underlie learning and memory, such as integration of sub-threshold synaptic responses and the conductance of back-propagating action potentials.

Accordingly, another embodiment of the invention features isolated TWIK-6 proteins and polypeptides having a TWIK-6 activity. Preferred proteins are TWIK-6 proteins having at least one transmembrane domain, and, preferably, a TWIK-6 activity. Other preferred proteins are TWIK-6 proteins having a TWIK-related ion channel domain and, preferably, a TWIK-6 activity. Other preferred proteins are TWIK-6 proteins having a P-loop domain and, preferably, a TWIK-6 activity. Other preferred proteins are TWIK-6 proteins having a potassium channel protein domain and, preferably, a TWIK-6 activity. Yet other preferred proteins are TWIK-6 proteins having at least one transmembrane domain, a TWIK-related ion channel domain, a P-loop domain, and a potassium channel protein domain and, preferably, a TWIK-6 activity.

Additional preferred proteins have at least one transmembrane domain, and one or more of a TWIK-related ion channel domain, a P-loop domain, and/or a potassium channel protein domain, and are, preferably, encoded by a nucleic acid molecule having a nucleotide sequence which hybridizes under stringent hybridization conditions to a nucleic acid molecule comprising the nucleotide sequence of SEQ ID NO:1 or 3 or SEQ ID NO:4 or 6.

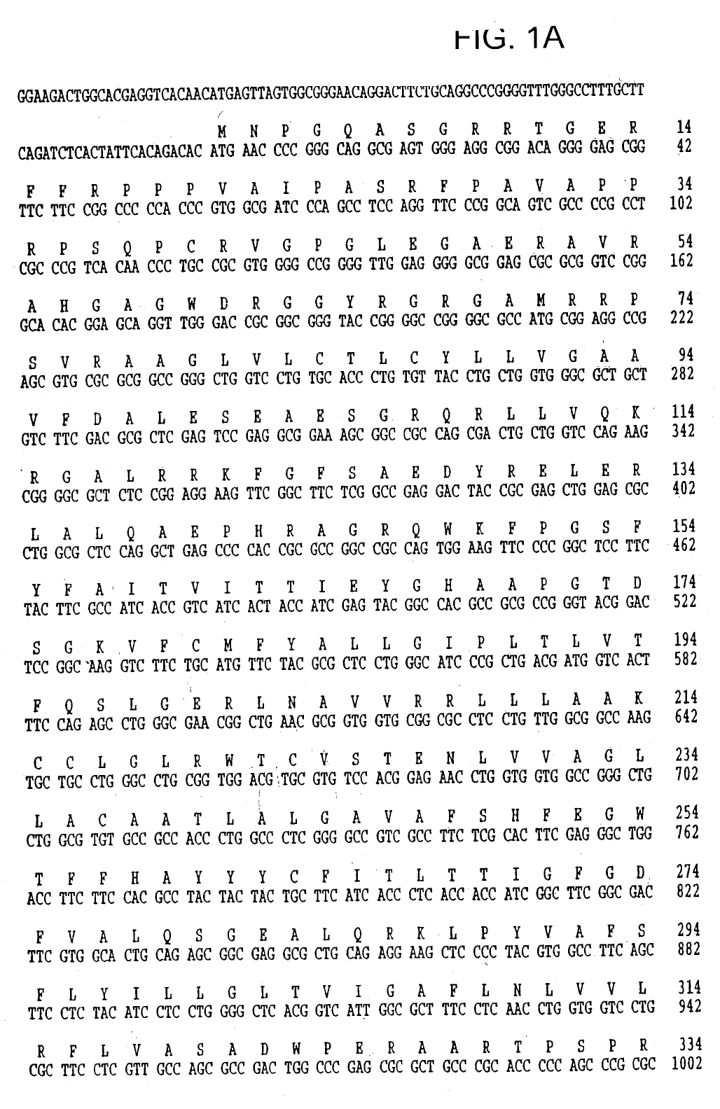

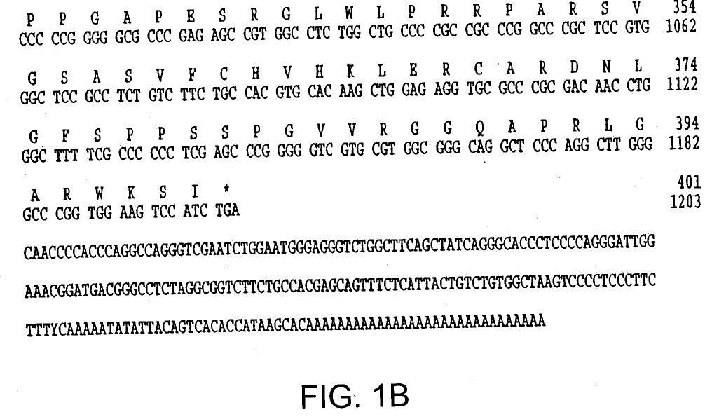

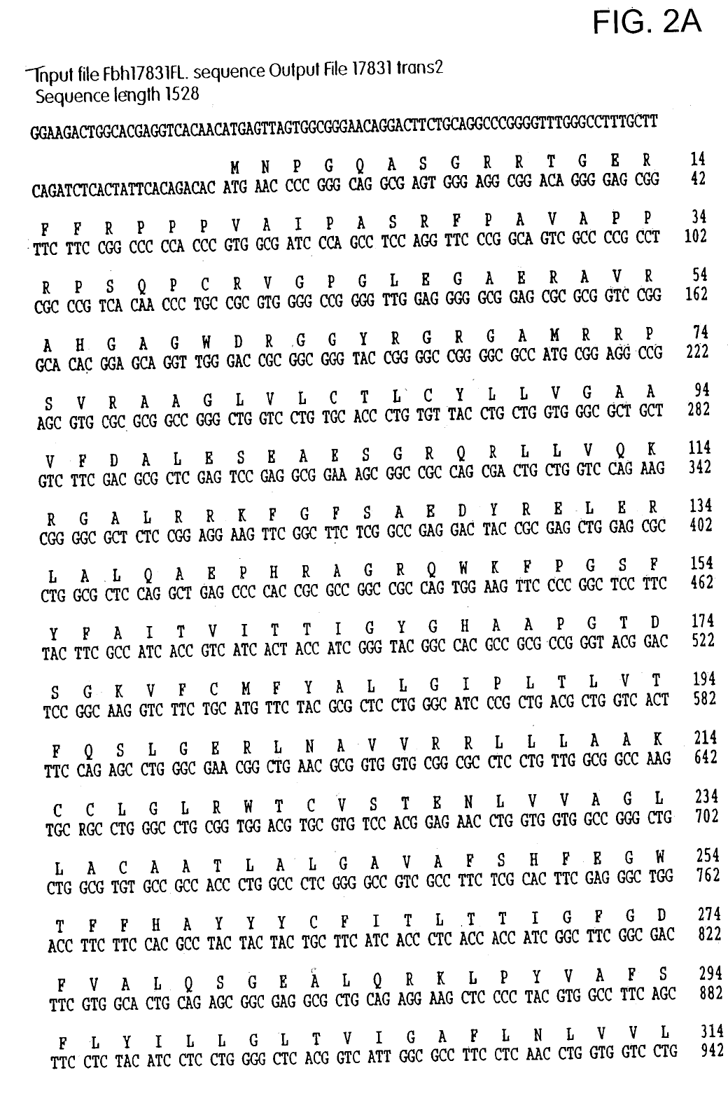

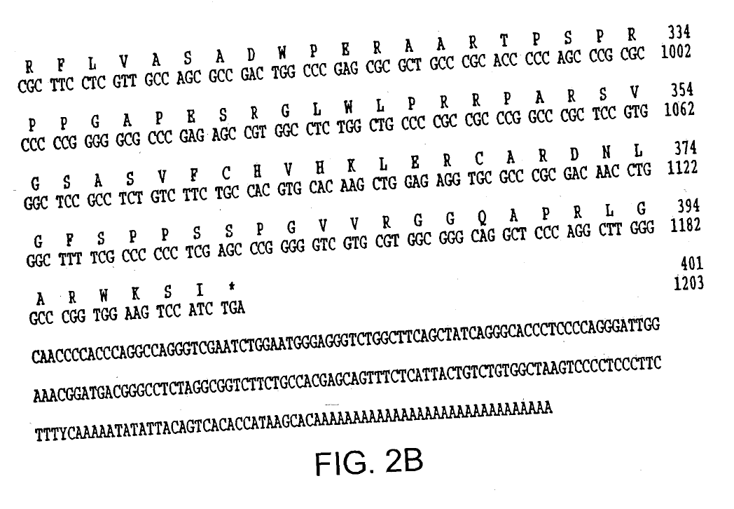

The nucleotide sequence of the isolated human TWIK-6 cDNA and the predicted amino acid sequence of the human TWIK-6 polypeptide are shown in FIGS. 1A-1B and in SEQ ID NOs:1 and 2, respectively. The nucleotide sequences of the human TWIK-6 containing an A to G polymorphism at nucleotide 596 and the predicted amino acid sequence of the human TWIK-6 polypeptide containing an E to G substitution at amino acid position 165 are shown in FIGS. 2A-2B and in SEQ ID NOs:4 and 6, respectively. A plasmid containing the nucleotide sequence encoding human TWIK-6 was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The human TWIK-6 gene, which is approximately 1528 nucleotides in length, encodes a protein having a molecular weight of approximately 45.3 kD and which is approximately 400 amino acid residues in length.

Various aspects of the invention are described in further detail in the following subsections:

I. Isolated Nucleic Acid Molecules

One aspect of the invention pertains to isolated nucleic acid molecules that encode TWIK-6 proteins or biologically active portions thereof, as well as nucleic acid fragments sufficient for use as hybridization probes to identify TWIK-6-encoding nucleic acid molecules (e.g., TWIK-6 mRNA) and fragments for use as PCR primers for the amplification or mutation of TWIK-6 nucleic acid molecules. As used herein, the term “nucleic acid molecule” is intended to include DNA molecules (e.g., cDNA or genomic DNA) and RNA molecules (e.g, mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TWIK-6 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1 or 3, or SEQ ID NO:4 or 6, or the nucleotide sequence of the DNA insert of the plasmid deposited with ATCC as Accession Number ______, or a portion thereof, can be isolated using standard molecular biology techniques and the sequence information provided herein. Using all or portion of the nucleic acid sequence of SEQ ID NO:1 or 3, or SEQ ID NO:4 or 6, or the nucleotide sequence of the DNA insert of the plasmid deposited with ATCC as Accession Number ______, as a hybridization probe, TWIK-6 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 or 3, or SEQ ID NO:4 or 6, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:1 or 3, or SEQ ID NO:4 or 6, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to TWIK-6 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1 or 4. The sequences of SEQ ID NO:1 and SEQ ID NO:4 correspond to alternate polymorphic forms of the human TWIK-6 cDNA. This cDNA comprises sequences encoding the human TWIK-6 protein (i.e., “the coding region”, from nucleotides 103-1305), as well as 5′ untranslated sequences (nucleotides 1-102) and 3′ untranslated sequences (nucleotides 1306-1528). Alternatively, the nucleic acid molecule can comprise only the coding region of SEQ ID NO:1 or 4 (e.g., nucleotides 103-1305, corresponding to SEQ ID NO:3 or 6, respectively).

In another preferred embodiment, an isolated nucleic acid molecule of the invention comprises a nucleic acid molecule which is a complement of the nucleotide sequence shown in SEQ ID NO:1 or 3, or SEQ ID NO:4 or 6, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:1 or 3, or SEQ ID NO:4 or 6, or the nucleotide sequence of the DNA insert of the plasmid deposited with ATCC as Accession Number ______, is one which is sufficiently complementary to the nucleotide sequence shown in SEQ ID NO:1 or 3, or SEQ ID NO:4 or 6, or the nucleotide sequence of the DNA insert of the plasmid deposited with ATCC as Accession Number ______, such that it can hybridize to the nucleotide sequence shown in SEQ ID NO:1 or 3, or SEQ ID NO:4 or 6, or the nucleotide sequence of the DNA insert of the plasmid deposited with ATCC as Accession Number ______, thereby forming a stable duplex.

In still another preferred embodiment, an isolated nucleic acid molecule of the present invention comprises a nucleotide sequence which is at least about 50%, 55%, 60%, 65%, 70%, 73%, 74%, 75%, 80%, 85%, 90%, 95%, 96%, 97%, 98%, 99% or more identical to the entire length of the nucleotide sequence shown in SEQ ID NO:1 or 3, or SEQ ID NO:4 or 6, or the entire length of the nucleotide sequence of the DNA insert of the plasmid deposited with ATCC as Accession Number ______, or a portion of any of these nucleotide sequences.

Moreover, the nucleic acid molecule of the invention can comprise only a portion of the nucleic acid sequence of SEQ ID NO:1 or 3, or SEQ ID NO:4 or 6, or the nucleotide sequence of the DNA insert of the plasmid deposited with ATCC as Accession Number ______, for example, a fragment which can be used as a probe or primer or a fragment encoding a portion of a TWIK-6 protein, e.g., a biologically active portion of a TWIK-6 protein. The nucleotide sequence determined from the cloning of the TWIK-6 gene allows for the generation of probes and primers designed for use in identifying and/or cloning other TWIK-6 family members, as well as TWIK-6 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 or 3, or the nucleotide sequence of the DNA insert of the plasmid deposited with ATCC as Accession Number ______, of an anti-sense sequence of SEQ ID NO:1 or 3, or SEQ ID NO:4 or 6, or the nucleotide sequence of the DNA insert of the plasmid deposited with ATCC as Accession Number ______, or of a naturally occurring allelic variant or mutant of SEQ ID NO:1 or 3, or SEQ ID NO:4 or 6, or the nucleotide sequence of the DNA insert of the plasmid deposited with ATCC as Accession Number ______. In one embodiment, a nucleic acid molecule of the present invention comprises a nucleotide sequence which is greater than 1050, 1051-1100, 1100-1150, 1150-1200, 1200-1250, 1250-1300, 1300-1350, 1350-1400, 1400-1450, 1450-1500 or more nucleotides in length and hybridizes under stringent hybridization conditions to a nucleic acid molecule of SEQ ID NO:1 or 3, or SEQ ID NO:4 or 6, or the nucleotide sequence of the DNA insert of the plasmid deposited with ATCC as Accession Number ______.

Probes based on the TWIK-6 nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a TWIK-6 protein, such as by measuring a level of a TWIK-6-encoding nucleic acid in a sample of cells from a subject e.g., detecting TWIK-6 mRNA levels or determining whether a genomic TWIK-6 gene has been mutated or deleted.

A nucleic acid fragment encoding a “biologically active portion of a TWIK-6 protein” can be prepared by isolating a portion of the nucleotide sequence of SEQ ID NO:1 or 3, or SEQ ID NO:4 or 6, or the nucleotide sequence of the DNA insert of the plasmid deposited with ATCC as Accession Number ______, which encodes a polypeptide having a TWIK-6 biological activity (the biological activities of the TWIK-6 proteins are described herein), expressing the encoded portion of the TWIK-6 protein (e.g., by recombinant expression in vitro) and assessing the activity of the encoded portion of the TWIK-6 protein.

The invention further encompasses nucleic acid molecules that differ from the nucleotide sequence shown in SEQ ID NO:1 or 3, or SEQ ID NO:4 or 6, or the nucleotide sequence of the DNA insert of the plasmid deposited with ATCC as Accession Number ______, due to degeneracy of the genetic code and thus encode the same TWIK-6 proteins as those encoded by the nucleotide sequence shown in SEQ ID NO:1 or 3, or SEQ ID NO:4 or 6, or the nucleotide sequence of the DNA insert of the plasmid deposited with ATCC as Accession Number ______. In another embodiment, an isolated nucleic acid molecule of the invention has a nucleotide sequence encoding a protein having an amino acid sequence shown in SEQ ID NO:2 or 5.

In addition to the TWIK-6 nucleotide sequences shown in SEQ ID NO:1 or 3, or SEQ ID NO:4 or 6, or the nucleotide sequence of the DNA insert of the plasmid deposited with ATCC as Accession Number ______, it will be appreciated by those skilled in the art that DNA sequence polymorphisms that lead to changes in the amino acid sequences of the TWIK-6 proteins may exist within a population (e.g., the human population). Such genetic polymorphism in the TWIK-6 genes may exist among individuals within a population due to natural allelic variation. As used herein, the terms “gene” and “recombinant gene” refer to nucleic acid molecules which include an open reading frame encoding a TWIK-6 protein, preferably a mammalian TWIK-6 protein, and can further include non-coding regulatory sequences, and introns.

Allelic variants of human TWIK-6 include both functional and non-functional TWIK-6 proteins. Functional allelic variants are naturally occurring amino acid sequence variants of the human TWIK-6 protein that maintain the ability to bind a TWIK-6 ligand or substrate and/or modulate cell proliferation and/or migration mechanisms. Functional allelic variants will typically contain only conservative substitution of one or more amino acids of SEQ ID NO:2 or 5, or substitution, deletion or insertion of non-critical residues in non-critical regions of the protein.

Non-functional allelic variants are naturally occurring amino acid sequence variants of the human TWIK-6 protein that do not have the ability to either bind a TWIK-6 ligand and/or modulate any of the TWIK-6 activities described herein. Non-functional allelic variants will typically contain a non-conservative substitution, a deletion, or insertion or premature truncation of the amino acid sequence of SEQ ID NO:2 or 5, or a substitution, insertion or deletion in critical residues or critical regions.

The present invention further provides non-human orthologues of the human TWIK-6 protein. Orthologues of the human TWIK-6 protein are proteins that are isolated from non-human organisms and possess the same TWIK-6 ligand binding and/or modulation of membrane excitability activities of the human TWIK-6 protein. Orthologues of the human TWIK-6 protein can readily be identified as comprising an amino acid sequence that is substantially identical to SEQ ID NO:2 or 5.

Moreover, nucleic acid molecules encoding other TWIK-6 family members and, thus, which have a nucleotide sequence which differs from the TWIK-6 sequences of SEQ ID NO:1 or 3, or SEQ ID NO:4 or 6, or the nucleotide sequence of the DNA insert of the plasmid deposited with ATCC as Accession Number ______ are intended to be within the scope of the invention. For example, another TWIK-6 cDNA can be identified based on the nucleotide sequence of human TWIK-6. Moreover, nucleic acid molecules encoding TWIK-6 proteins from different species, and which, thus, have a nucleotide sequence which differs from the TWIK-6 sequences of SEQ ID NO:1 or 3, or SEQ ID NO:4 or 6, or the nucleotide sequence of the DNA insert of the plasmid deposited with ATCC as Accession Number ______ are intended to be within the scope of the invention. For example, a mouse TWIK-6 cDNA can be identified based on the nucleotide sequence of a human TWIK-6.

Nucleic acid molecules corresponding to natural allelic variants and homologues of the TWIK-6 cDNAs of the invention can be isolated based on their homology to the TWIK-6 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the TWIK-6 cDNAs of the invention can further be isolated by mapping to the same chromosome or locus as the TWIK-6 gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1 or 3, or SEQ ID NO:4 or 6, or the nucleotide sequence of the DNA insert of the plasmid deposited with ATCC as Accession Number ______. In other embodiment, the nucleic acid is at least 1050, 1051-1100, 1100-1150, 1150-1200, 1200-1250, 1250-1300, 1300-1350, 1350-1400, 1400-1450, 1450-1500, or more nucleotides in length. As used herein, the term “hybridizes under stringent conditions” is intended to describe conditions for hybridization and washing under which nucleotide sequences at least 60% identical to each other typically remain hybridized to each other. Preferably, the conditions are such that sequences at least about 70%, more preferably at least about 80%, even more preferably at least about 85% or 90% identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50° C., preferably at 55° C., more preferably at 60° C., and even more preferably at 65° C. Ranges intermediate to the above-recited values, e.g., at 60-65° C. or at 55-60° C. are also intended to be encompassed by the present invention. Preferably, an isolated nucleic acid molecule of the invention that hybridizes under stringent conditions to the sequence of SEQ ID NO:1 or 3, or SEQ ID NO:4 or 6, and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the TWIK-6 sequences that may exist in the population, the skilled artisan will further appreciate that changes can be introduced by mutation into the nucleotide sequences of SEQ ID NO:1 or 3, or SEQ ID NO:4 or 6, or the nucleotide sequence of the DNA insert of the plasmid deposited with ATCC as Accession Number ______, thereby leading to changes in the amino acid sequence of the encoded TWIK-6 proteins, without altering the functional ability of the TWIK-6 proteins. For example, nucleotide substitutions leading to amino acid substitutions at “non-essential” amino acid residues can be made in the sequence of SEQ ID NO:1 or 3, or SEQ ID NO:4 or 6, or the nucleotide sequence of the DNA insert of the plasmid deposited with ATCC as Accession Number ______. A “non-essential” amino acid residue is a residue that can be altered from the wild-type sequence of TWIK-6 (e.g., the sequence of SEQ ID NO:2 or 5) without altering the biological activity, whereas an “essential” amino acid residue is required for biological activity. For example, amino acid residues that are conserved among the TWIK-6 proteins of the present invention, e.g., those present in a transmembrane domain, are predicted to be particularly unamenable to alteration. Furthermore, additional amino acid residues that are conserved between the TWIK-6 proteins of the present invention and other members of the TWIK family are not likely to be amenable to alteration.

Accordingly, another aspect of the invention pertains to nucleic acid molecules encoding TWIK-6 proteins that contain changes in amino acid residues that are not essential for activity. Such TWIK-6 proteins differ in amino acid sequence from SEQ ID NO:2 or 5, yet retain biological activity. In one embodiment, the isolated nucleic acid molecule comprises a nucleotide sequence encoding a protein, wherein the protein comprises an amino acid sequence at least about 50%, 55%, 60%, 65%, 67%, 68%, 70%, 75%, 80%, 85%, 90%, 95%, 96%, 97%, 98%, 99% or more identical to SEQ ID NO:2 or 5.

An isolated nucleic acid molecule encoding a TWIK-6 protein identical to the protein of SEQ ID NO:2 or 5, can be created by introducing one or more nucleotide substitutions, additions or deletions into the nucleotide sequence of SEQ ID NO:1 or 3, or SEQ ID NO:4 or 6, or the nucleotide sequence of the DNA insert of the plasmid deposited with ATCC as Accession Number ______, such that one or more amino acid substitutions, additions or deletions are introduced into the encoded protein. Mutations can be introduced into SEQ ID NO:1 or 3, or SEQ ID NO:4 or 6, or the nucleotide sequence of the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a TWIK-6 protein is preferably replaced with another amino acid residue from the same side chain family. Alternatively, in another embodiment, mutations can be introduced randomly along all or part of a TWIK-6 coding sequence, such as by saturation mutagenesis, and the resultant mutants can be screened for TWIK-6 biological activity to identify mutants that retain activity. Following mutagenesis of SEQ ID NO:1 or 3, or SEQ ID NO:4 or 6, or the nucleotide sequence of the DNA insert of the plasmid deposited with ATCC as Accession Number ______, the encoded protein can be expressed recombinantly and the activity of the protein can be determined.

In a preferred embodiment, a mutant TWIK-6 protein can be assayed for the ability to (1) interact with a non-TWIK protein molecule; (2) activate a TWIK-dependent signal transduction pathway; (3) modulate the release of neurotransmitters; (4) modulate membrane excitability; (5) influence the resting potential of membranes, wave forms and frequencies of action potentials, and thresholds of excitation, and (6) modulate processes which underlie learning and memory, such as integration of sub-threshold synaptic responses and the conductance of back-propagating action potentials.

In addition to the nucleic acid molecules encoding TWIK-6 proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TWIK-6 coding strand, or to only a portion thereof. In one embodiment, an antisense nucleic acid molecule is antisense to a “coding region” of the coding strand of a nucleotide sequence encoding TWIK-6. The term “coding region” refers to the region of the nucleotide sequence comprising codons which are translated into amino acid residues (e.g., the coding region of human TWIK-6 corresponds to SEQ ID NO:3 or 6). In another embodiment, the antisense nucleic acid molecule is antisense to a “noncoding region” of the coding strand of a nucleotide sequence encoding TWIK-6. The term “noncoding region” refers to 5′ and 3′ sequences which flank the coding region that are not translated into amino acids (i.e., also referred to as 5′ and 3′ untranslated regions).

Given the coding strand sequences encoding TWIK-6 disclosed herein (e.g., SEQ ID NO:3 or 6), antisense nucleic acids of the invention can be designed according to the rules of Watson and Crick base pairing. The antisense nucleic acid molecule can be complementary to the entire coding region of TWIK-6 mRNA, but more preferably is an oligonucleotide which is antisense to only a portion of the coding or noncoding region of TWIK-6 mRNA. For example, the antisense oligonucleotide can be complementary to the region surrounding the translation start site of TWIK-6 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a TWIK-6 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave TWIK-6 mRNA transcripts to thereby inhibit translation of TWIK-6 mRNA. A ribozyme having specificity for a TWIK-6-encoding nucleic acid can be designed based upon the nucleotide sequence of a TWIK-6 cDNA disclosed herein (i.e., SEQ ID NO:1 or 3, or SEQ ID NO:4 or 6, or the nucleotide sequence of the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in a TWIK-6-encoding mRNA. See, e.g., Cech et al. U.S. Pat. No. 4,987,071; and Cech et al. U.S. Pat. No. 5,116,742. Alternatively, TWIK-6 mRNA can be used to select a catalytic RNA having a specific ribonuclease activity from a pool of RNA molecules. See, e.g., Bartel, D. and Szostak, J. W. (1993) Science 261:1411-1418.

Alternatively, TWIK-6 gene expression can be inhibited by targeting nucleotide sequences complementary to the regulatory region of the TWIK-6 (e.g., the TWIK-6 promoter and/or enhancers; e.g., nucleotides 1-102 of SEQ ID NO:1 or 4) to form triple helical structures that prevent transcription of the TWIK-6 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the TWIK-6 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of TWIK-6 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of TWIK-6 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of TWIK-6 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of TWIK-6 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous TWIK-6 gene within a cell line or microorganism may be modified by inserting a heterologous DNA regulatory element into the genome of a stable cell line or cloned microorganism such that the inserted regulatory element is operatively linked with the endogenous TWIK-6 gene. For example, an endogenous TWIK-6 gene which is normally “transcriptionally silent”, i.e., a TWIK-6 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous TWIK-6 gene may be activated by insertion of a promiscuous regulatory element that works across cell types.

A heterologous regulatory element may be inserted into a stable cell line or cloned microorganism, such that it is operatively linked with an endogenous TWIK-6 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated TWIK-6 Proteins and Anti-TWIK-6 Antibodies

One aspect of the invention pertains to isolated TWIK-6 proteins, and biologically active portions thereof, as well as polypeptide fragments suitable for use as immunogens to raise anti-TWIK-6 antibodies. In one embodiment, native TWIK-6 proteins can be isolated from cells or tissue sources by an appropriate purification scheme using standard protein purification techniques. In another embodiment, TWIK-6 proteins are produced by recombinant DNA techniques. Alternative to recombinant expression, a TWIK-6 protein or polypeptide can be synthesized chemically using standard peptide synthesis techniques.

An “isolated” or “purified” protein or biologically active portion thereof is substantially free of cellular material or other contaminating proteins from the cell or tissue source from which the TWIK-6 protein is derived, or substantially free from chemical precursors or other chemicals when chemically synthesized. The language “substantially free of cellular material” includes preparations of TWIK-6 protein in which the protein is separated from cellular components of the cells from which it is isolated or recombinantly produced. In one embodiment, the language “substantially free of cellular material” includes preparations of TWIK-6 protein having less than about 30% (by dry weight) of non-TWIK-6 protein (also referred to herein as a “contaminating protein”), more preferably less than about 20% of non-TWIK-6 protein, still more preferably less than about 10% of non-TWIK-6 protein, and most preferably less than about 5% non-TWIK-6 protein. When the TWIK-6 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of TWIK-6 protein in which the protein is separated from chemical precursors or other chemicals which are involved in the synthesis of the protein. In one embodiment, the language “substantially free of chemical precursors or other chemicals” includes preparations of TWIK-6 protein having less than about 30% (by dry weight) of chemical precursors or non-TWIK-6 chemicals, more preferably less than about 20% chemical precursors or non-TWIK-6 chemicals, still more preferably less than about 10% chemical precursors or non-TWIK-6 chemicals, and most preferably less than about 5% chemical precursors or non-TWIK-6 chemicals.

As used herein, a “biologically active portion” of a TWIK-6 protein includes a fragment of a TWIK-6 protein which participates in an interaction between a TWIK-6 molecule and a non-TWIK-6 molecule. Biologically active portions of a TWIK-6 protein include peptides comprising amino acid sequences sufficiently identical to or derived from the amino acid sequence of the TWIK-6 protein, e.g., the amino acid sequence shown in SEQ ID NO:2 or 5, which include less amino acids than the full length TWIK-6 proteins, and exhibit at least one activity of a TWIK-6 protein. Typically, biologically active portions comprise a domain or motif with at least one activity of the TWIK-6 protein, e.g., modulating membrane excitability. A biologically active portion of a TWIK-6 protein can be a polypeptide which is, for example, 25, 50, 75, 100, 125, 150, 175, 200 or more amino acids in length. Biologically active portions of a TWIK-6 protein can be used as targets for developing agents which modulate a TWIK-6 mediated activity, e.g., modulation of membrane excitability.

In one embodiment, a biologically active portion of a TWIK-6 protein comprises at least one transmembrane domain. It is to be understood that a preferred biologically active portion of a TWIK-6 protein of the present invention may contain at least one transmembrane domain and one or more of the following domains: a TWIK-related ion channel domain, a P-loop domain, and a potassium channel protein domain. Moreover, other biologically active portions, in which other regions of the protein are deleted, can be prepared by recombinant techniques and evaluated for one or more of the functional activities of a native TWIK-6 protein.

In a preferred embodiment, the TWIK-6 protein has an amino acid sequence shown in SEQ ID NO:2 or 5. In other embodiments, the TWIK-6 protein is substantially identical to SEQ ID NO:2 or 5, and retains the functional activity of the protein of SEQ ID NO:2 or 5, yet differs in amino acid sequence due to natural allelic variation or mutagenesis, as described in detail in subsection I above. Accordingly, in another embodiment, the TWIK-6 protein is a protein which comprises an amino acid sequence at least about 50%, 55%, 60%, 65%, 67%, 68%, 70%, 75%, 80%, 85%, 90%, 95%, 96%, 97%, 98%, 99% or more identical to SEQ ID NO:2 or 5.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the TWIK-6 amino acid sequence of SEQ ID NO:2 or 5 having 400 amino acid residues, at least 50, preferably at least 100, more preferably at least 150, even more preferably at least 200, and even more preferably at least 300 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to TWIK-6 nucleic acid molecules of the invention. BLAST protein searches can be performed with the XBLAST program, score=100, wordlength=3 to obtain amino acid sequences homologous to TWIK-6 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides TWIK-6 chimeric or fusion proteins. As used herein, a TWIK-6 “chimeric protein” or “fusion protein” comprises a TWIK-6 polypeptide operatively linked to a non-TWIK-6 polypeptide. An “TWIK-6 polypeptide” refers to a polypeptide having an amino acid sequence corresponding to TWIK-6, whereas a “non-TWIK-6 polypeptide” refers to a polypeptide having an amino acid sequence corresponding to a protein which is not substantially homologous to the TWIK-6 protein, e.g., a protein which is different from the TWIK-6 protein and which is derived from the same or a different organism. Within a TWIK-6 fusion protein the TWIK-6 polypeptide can correspond to all or a portion of a TWIK-6 protein. In a preferred embodiment, a TWIK-6 fusion protein comprises at least one biologically active portion of a TWIK-6 protein. In another preferred embodiment, a TWIK-6 fusion protein comprises at least two biologically active portions of a TWIK-6 protein. Within the fusion protein, the term “operatively linked” is intended to indicate that the TWIK-6 polypeptide and the non-TWIK-6 polypeptide are fused in-frame to each other. The non-TWIK-6 polypeptide can be fused to the N-terminus or C-terminus of the TWIK-6 polypeptide.

For example, in one embodiment, the fusion protein is a GST-TWIK-6 fusion protein in which the TWIK-6 sequences are fused to the C-terminus of the GST sequences. Such fusion proteins can facilitate the purification of recombinant TWIK-6.

In another embodiment, the fusion protein is a TWIK-6 protein containing a heterologous signal sequence at its N-terminus. In certain host cells (e.g., mammalian host cells), expression and/or secretion of TWIK-6 can be increased through use of a heterologous signal sequence.

The TWIK-6 fusion proteins of the invention can be incorporated into pharmaceutical compositions and administered to a subject in vivo. The TWIK-6 fusion proteins can be used to affect the bioavailability of a TWIK-6 substrate. Use of TWIK-6 fusion proteins may be useful therapeutically for the treatment of disorders caused by, for example, (i) aberrant modification or mutation of a gene encoding a TWIK-6 protein; (ii) mis-regulation of the TWIK-6 gene; and (iii) aberrant post-translational modification of a TWIK-6 protein.

Moreover, the TWIK-6-fusion proteins of the invention can be used as immunogens to produce anti-TWIK-6 antibodies in a subject, to purify TWIK-6 ligands and in screening assays to identify molecules which inhibit the interaction of TWIK-6 with a TWIK-6 substrate.

Preferably, a TWIK-6 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A TWIK-6-encoding nucleic acid can be cloned into such an expression vector such that the fusion moiety is linked in-frame to the TWIK-6 protein.

The present invention also pertains to variants of the TWIK-6 proteins which function as either TWIK-6 agonists (mimetics) or as TWIK-6 antagonists. Variants of the TWIK-6 proteins can be generated by mutagenesis, e.g., discrete point mutation or truncation of a TWIK-6 protein. An agonist of the TWIK-6 proteins can retain substantially the same, or a subset, of the biological activities of the naturally occurring form of a TWIK-6 protein. An antagonist of a TWIK-6 protein can inhibit one or more of the activities of the naturally occurring form of the TWIK-6 protein by, for example, competitively modulating a TWIK-6-mediated activity of a TWIK-6 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the TWIK-6 protein.

In one embodiment, variants of a TWIK-6 protein which function as either TWIK-6 agonists (mimetics) or as TWIK-6 antagonists can be identified by screening combinatorial libraries of mutants, e.g., truncation mutants, of a TWIK-6 protein for TWIK-6 protein agonist or antagonist activity. In one embodiment, a variegated library of TWIK-6 variants is generated by combinatorial mutagenesis at the nucleic acid level and is encoded by a variegated gene library. A variegated library of TWIK-6 variants can be produced by, for example, enzymatically ligating a mixture of synthetic oligonucleotides into gene sequences such that a degenerate set of potential TWIK-6 sequences is expressible as individual polypeptides, or alternatively, as a set of larger fusion proteins (e.g., for phage display) containing the set of TWIK-6 sequences therein. There are a variety of methods which can be used to produce libraries of potential TWIK-6 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TWIK-6 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a TWIK-6 protein coding sequence can be used to generate a variegated population of TWIK-6 fragments for screening and subsequent selection of variants of a TWIK-6 protein. In one embodiment, a library of coding sequence fragments can be generated by treating a double stranded PCR fragment of a TWIK-6 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the TWIK-6 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TWIK-6 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify TWIK-6 variants (Arkin and Youvan (1992) Proc. Natl. Acad. Sci. USA 89:7811-7815; Delagrave et al. (1993) Protein Engineering 6(3): 327-331).

In one embodiment, cell based assays can be exploited to analyze a variegated TWIK-6 library. For example, a library of expression vectors can be transfected into a cell line, e.g., a neuronal cell line, which ordinarily responds to a TWIK-6 ligand in a particular TWIK-6 ligand-dependent manner. The transfected cells are then contacted with a TWIK-6 ligand and the effect of expression of the mutant on, e.g., membrane excitability of TWIK-6 can be detected. Plasmid DNA can then be recovered from the cells which score for inhibition, or alternatively, potentiation of signaling by the TWIK-6 ligand, and the individual clones further characterized.

An isolated TWIK-6 protein, or a portion or fragment thereof, can be used as an immunogen to generate antibodies that bind TWIK-6 using standard techniques for polyclonal and monoclonal antibody preparation. A full-length TWIK-6 protein can be used or, alternatively, the invention provides antigenic peptide fragments of TWIK-6 for use as immunogens. The antigenic peptide of TWIK-6 comprises at least 8 amino acid residues of the amino acid sequence shown in SEQ ID NO:2 or 5 and encompasses an epitope of TWIK-6 such that an antibody raised against the peptide forms a specific immune complex with TWIK-6. Preferably, the antigenic peptide comprises at least 10 amino acid residues, more preferably at least 15 amino acid residues, even more preferably at least 20 amino acid residues, and most preferably at least 30 amino acid residues.

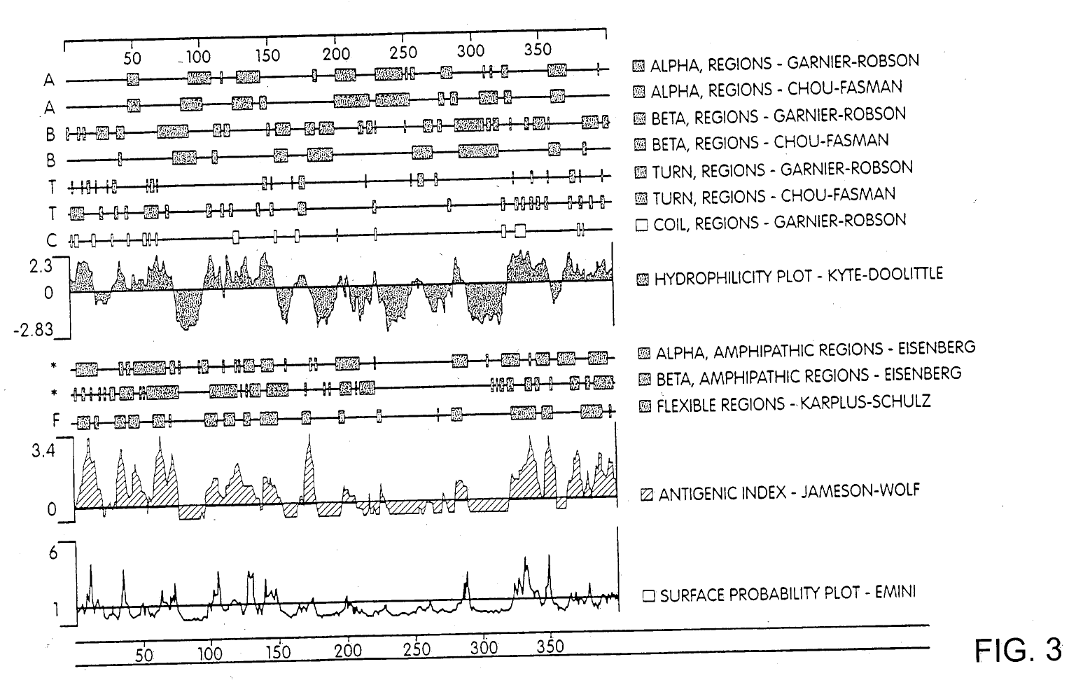

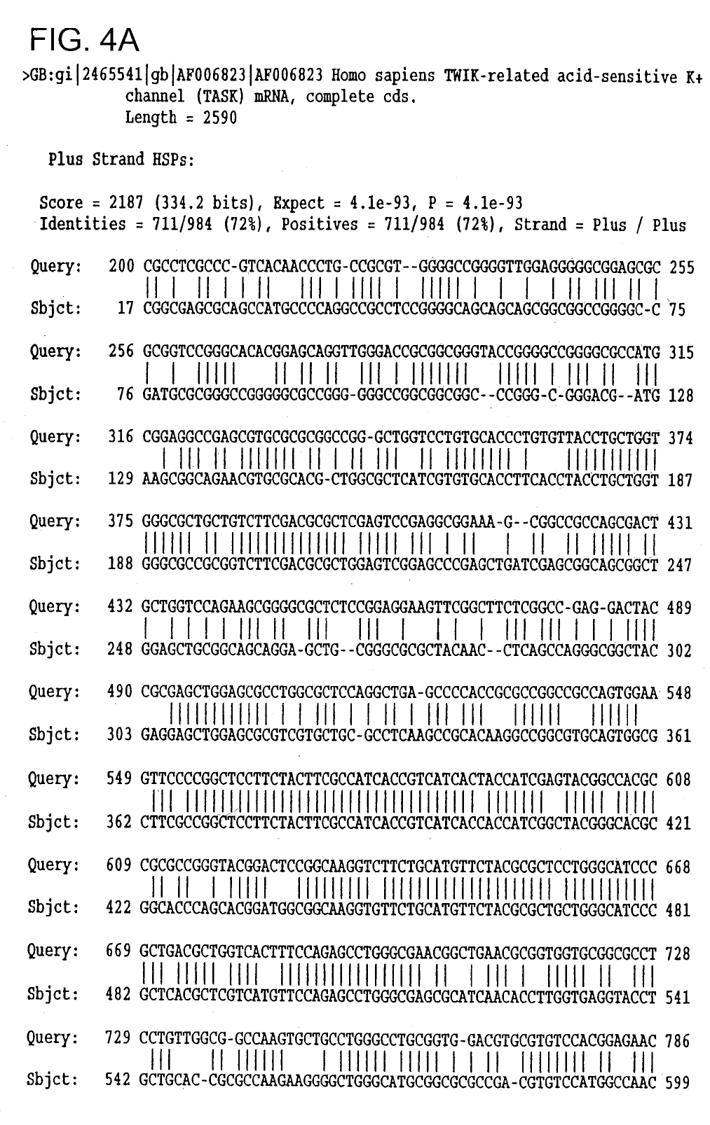

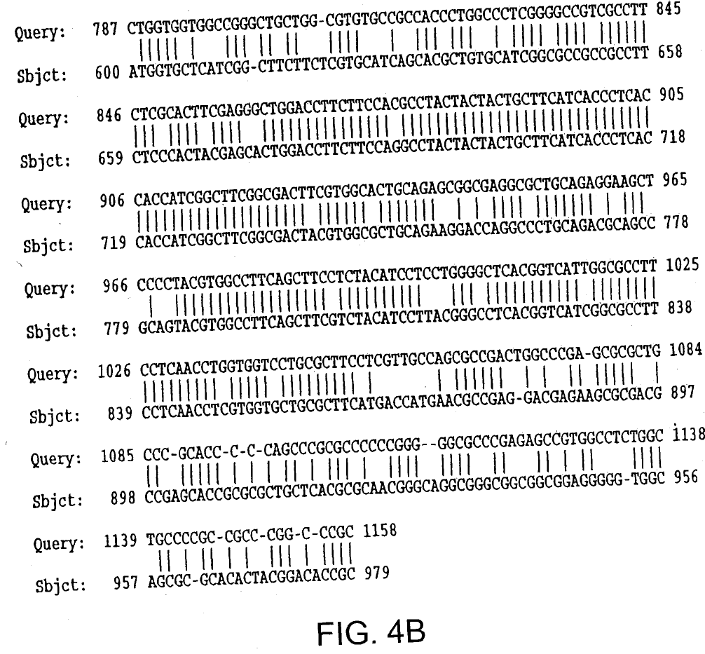

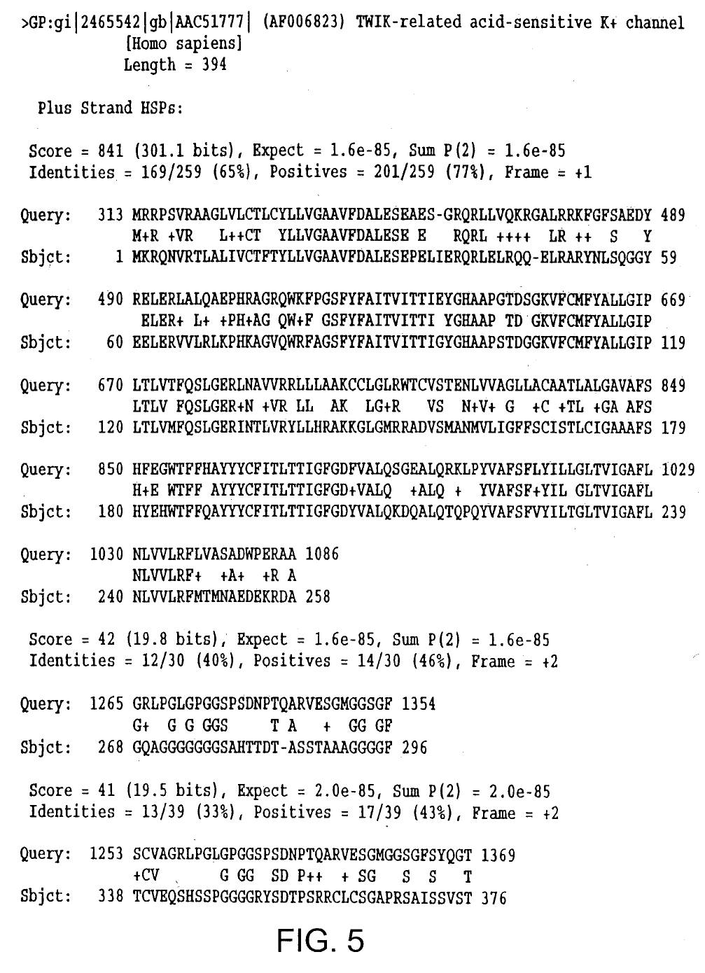

Preferred epitopes encompassed by the antigenic peptide are regions of TWIK-6 that are located on the surface of the protein, e.g., hydrophilic regions, as well as regions with high antigenicity (see, for example, FIG. 3).

A TWIK-6 immunogen typically is used to prepare antibodies by immunizing a suitable subject, (e.g., rabbit, goat, mouse or other mammal) with the immunogen. An appropriate immunogenic preparation can contain, for example, recombinantly expressed TWIK-6 protein or a chemically synthesized TWIK-6 polypeptide. The preparation can further include an adjuvant, such as Freund's complete or incomplete adjuvant, or similar immunostimulatory agent. Immunization of a suitable subject with an immunogenic TWIK-6 preparation induces a polyclonal anti-TWIK-6 antibody response.

Accordingly, another aspect of the invention pertains to anti-TWIK-6 antibodies. The term “antibody” as used herein refers to immunoglobulin molecules and immunologically active portions of immunoglobulin molecules, i.e., molecules that contain an antigen binding site which specifically binds (immunoreacts with) an antigen, such as TWIK-6. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind TWIK-6. The term “monoclonal antibody” or “monoclonal antibody composition”, as used herein, refers to a population of antibody molecules that contain only one species of an antigen binding site capable of immunoreacting with a particular epitope of TWIK-6. A monoclonal antibody composition thus typically displays a single binding affinity for a particular TWIK-6 protein with which it immunoreacts.

Polyclonal anti-TWIK-6 antibodies can be prepared as described above by immunizing a suitable subject with a TWIK-6 immunogen. The anti-TWIK-6 antibody titer in the immunized subject can be monitored over time by standard techniques, such as with an enzyme linked immunosorbent assay (ELISA) using immobilized TWIK-6. If desired, the antibody molecules directed against TWIK-6 can be isolated from the mammal (e.g., from the blood) and further purified by well known techniques, such as protein A chromatography to obtain the IgG fraction. At an appropriate time after immunization, e.g., when the anti-TWIK-6 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a TWIK-6 immunogen as described above, and the culture supernatants of the resulting hybridoma cells are screened to identify a hybridoma producing a monoclonal antibody that binds TWIK-6.

Any of the many well known protocols used for fusing lymphocytes and immortalized cell lines can be applied for the purpose of generating an anti-TWIK-6 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind TWIK-6, e.g., using a standard ELISA assay.

Alternative to preparing monoclonal antibody-secreting hybridomas, a monoclonal anti-TWIK-6 antibody can be identified and isolated by screening a recombinant combinatorial immunoglobulin library (e.g., an antibody phage display library) with TWIK-6 to thereby isolate immunoglobulin library members that bind TWIK-6. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod Hybridomas 3:81-85; Huse et al. (1989) Science 246:1275-1281; Griffiths et al. (1993) EMBO J 12:725-734; Hawkins et al. (1992) J. Mol. Biol 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-TWIK-6 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-TWIK-6 antibody (e.g, monoclonal antibody) can be used to isolate TWIK-6 by standard techniques, such as affinity chromatography or immunoprecipitation. An anti-TWIK-6 antibody can facilitate the purification of natural TWIK-6 from cells and of recombinantly produced TWIK-6 expressed in host cells. Moreover, an anti-TWIK-6 antibody can be used to detect TWIK-6 protein (e.g., in a cellular lysate or cell supernatant) in order to evaluate the abundance and pattern of expression of the TWIK-6 protein. Anti-TWIK-6 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

II. Recombinant Expression Vectors and Host Cells

Another aspect of the invention pertains to vectors, preferably expression vectors, containing a nucleic acid encoding a TWIK-6 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., TWIK-6 proteins, mutant forms of TWIK-6 proteins, fusion proteins, and the like).

The recombinant expression vectors of the invention can be designed for expression of TWIK-6 proteins in prokaryotic or eukaryotic cells. For example, TWIK-6 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in TWIK-6 activity assays, (e.g., direct assays or competitive assays described in detail below), or to generate antibodies specific for TWIK-6 proteins, for example. In a preferred embodiment, a TWIK-6 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the TWIK-6 expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corporation, San Diego, Calif.).

Alternatively, TWIK-6 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to TWIK-6 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a TWIK-6 nucleic acid molecule of the invention is introduced, e.g., a TWIK-6 nucleic acid molecule within a recombinant expression vector or a TWIK-6 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a TWIK-6 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a TWIK-6 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a TWIK-6 protein. Accordingly, the invention further provides methods for producing a TWIK-6 protein using the host cells of the invention. In one embodiment, the method comprises culturing the host cell of the invention (into which a recombinant expression vector encoding a TWIK-6 protein has been introduced) in a suitable medium such that a TWIK-6 protein is produced. In another embodiment, the method further comprises isolating a TWIK-6 protein from the medium or the host cell.

The host cells of the invention can also be used to produce non-human transgenic animals. For example, in one embodiment, a host cell of the invention is a fertilized oocyte or an embryonic stem cell into which TWIK-6-coding sequences have been introduced. Such host cells can then be used to create non-human transgenic animals in which exogenous TWIK-6 sequences have been introduced into their genome or homologous recombinant animals in which endogenous TWIK-6 sequences have been altered. Such animals are useful for studying the function and/or activity of a TWIK-6 and for identifying and/or evaluating modulators of TWIK-6 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous TWIK-6 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a TWIK-6-encoding nucleic acid into the male pronuclei of a fertilized oocyte, e.g., by microinjection, retroviral infection, and allowing the oocyte to develop in a pseudopregnant female foster animal. The TWIK-6 cDNA sequence of SEQ ID NO:1 or 4 can be introduced as a transgene into the genome of a non-human animal. Alternatively, a nonhuman homologue of a human TWIK-6 gene, such as a mouse or rat TWIK-6 gene, can be used as a transgene. Alternatively, a TWIK-6 gene homologue, such as another TWIK-6 family member, can be isolated based on hybridization to the TWIK-6 cDNA sequences of SEQ ID NO:1 or 3, or SEQ ID NO:4 or 6, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to a TWIK-6 transgene to direct expression of a TWIK-6 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a TWIK-6 transgene in its genome and/or expression of TWIK-6 mRNA in tissues or cells of the animals. A transgenic founder animal can then be used to breed additional animals carrying the transgene. Moreover, transgenic animals carrying a transgene encoding a TWIK-6 protein can further be bred to other transgenic animals carrying other transgenes.

To create a homologous recombinant animal, a vector is prepared which contains at least a portion of a TWIK-6 gene into which a deletion, addition or substitution has been introduced to thereby alter, e.g., functionally disrupt, the TWIK-6 gene. The TWIK-6 gene can be a human gene (e.g., the cDNA of SEQ ID NO:3 or 6), but more preferably, is a non-human homologue of a human TWIK-6 gene (e.g., a cDNA isolated by stringent hybridization with the nucleotide sequence of SEQ ID NO:1 or 4). For example, a mouse TWIK-6 gene can be used to construct a homologous recombination nucleic acid molecule, e.g., a vector, suitable for altering an endogenous TWIK-6 gene in the mouse genome. In a preferred embodiment, the homologous recombination nucleic acid molecule is designed such that, upon homologous recombination, the endogenous TWIK-6 gene is functionally disrupted (i.e., no longer encodes a functional protein; also referred to as a “knock out” vector). Alternatively, the homologous recombination nucleic acid molecule can be designed such that, upon homologous recombination, the endogenous TWIK-6 gene is mutated or otherwise altered but still encodes functional protein (e.g., the upstream regulatory region can be altered to thereby alter the expression of the endogenous TWIK-6 protein). In the homologous recombination nucleic acid molecule, the altered portion of the TWIK-6 gene is flanked at its 5′ and 3′ ends by additional nucleic acid sequence of the TWIK-6 gene to allow for homologous recombination to occur between the exogenous TWIK-6 gene carried by the homologous recombination nucleic acid molecule and an endogenous TWIK-6 gene in a cell, e.g., an embryonic stem cell. The additional flanking TWIK-6 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced TWIK-6 gene has homologously recombined with the endogenous TWIK-6 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The TWIK-6 nucleic acid molecules, fragments of TWIK-6 proteins, and anti-TWIK-6 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a TWIK-6 protein or an anti-TWIK-6 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramnycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a TWIK-6 protein of the invention has one or more of the following activities: (1) interacting with a non-TWIK protein molecule; (2) activating a TWIK-dependent signal transduction pathway; (3) modulating the release of neurotransmitters; (4) modulating membrane excitability; (5) influencing the resting potential of membranes, wave forms and frequencies of action potentials, and thresholds of excitation, and (6) modulating processes which underlie learning and memory, such as integration of sub-threshold synaptic responses and the conductance of back-propagating action potentials.

The isolated nucleic acid molecules of the invention can be used, for example, to express TWIK-6 protein (e.g., via a recombinant expression vector in a host cell in gene therapy applications), to detect TWIK-6 mRNA (e.g., in a biological sample) or a genetic alteration in a TWIK-6 gene, and to modulate TWIK-6 activity, as described further below. The TWIK-6 proteins can be used to treat disorders characterized by insufficient or excessive production of a TWIK-6 substrate or production of TWIK-6 inhibitors. In addition, the TWIK-6 proteins can be used to screen for naturally occurring TWIK-6 substrates, to screen for drugs or compounds which modulate TWIK-6 activity, as well as to treat disorders characterized by insufficient or excessive production of TWIK-6 protein or production of TWIK-6 protein forms which have decreased, aberrant or unwanted activity compared to TWIK-6 wild type protein (e.g., proliferative disorders, CNS disorders such as cognitive and neurodegenerative disorders (e.g., Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, and bipolar affective disorder (e.g., severe bipolar affective (mood) disorder (BP-1) and bipolar affective neurological disorders (e.g., migraine and obesity)); cardiac disorders (e.g., arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia), muscular disorders (e.g., paralysis, muscle weakness (e.g., ataxia, myotonia, and myokymia), muscular dystrophy (e.g., Duchenne muscular dystrophy or myotonic dystrophy), spinal muscular atrophy, congenital myopathies, central core disease, rod myopathy, central nuclear myopathy, Lambert-Eaton syndrome, denervation, and infantile spinal muscular atrophy (Werdnig-Hoffman disease), and cellular growth, differentiation, or migration disorders (e.g., cancer, e.g., carcinoma, sarcoma, or leukemia; tumor angiogenesis and metastasis; skeletal dysplasia; neuronal deficiencies resulting from impaired neural induction and patterning; hepatic disorders; cardiovascular disorders; and hematopoietic and/or myeloproliferative disorders). Moreover, the anti-TWIK-6 antibodies of the invention can be used to detect and isolate TWIK-6 proteins, regulate the bioavailability of TWIK-6 proteins, and modulate TWIK-6 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to TWIK-6 proteins, have a stimulatory or inhibitory effect on, for example, TWIK-6 expression or TWIK-6 activity, or have a stimulatory or inhibitory effect on, for example, the expression or activity of TWIK-6 substrate.

In one embodiment, the invention provides assays for screening candidate or test compounds which are substrates of a TWIK-6 protein or polypeptide or biologically active portion thereof. In another embodiment, the invention provides assays for screening candidate or test compounds which bind to or modulate the activity of a TWIK-6 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a TWIK-6 protein or biologically active portion thereof is contacted with a test compound and the ability of the test compound to modulate TWIK-6 activity is determined. Determining the ability of the test compound to modulate TWIK-6 activity can be accomplished by monitoring, for example, the release of a neurotransmitter from a cell which expresses TWIK-6. The cell, for example, can be of mammalian origin, e.g., a neuronal cell or a thymus cell. The ability of the test compound to modulate TWIK-6 binding to a substrate or to bind to TWIK-6 can also be determined. Determining the ability of the test compound to modulate TWIK-6 binding to a substrate can be accomplished, for example, by coupling the TWIK-6 substrate with a radioisotope or enzymatic label such that binding of the TWIK-6 substrate to TWIK-6 can be determined by detecting the labeled TWIK-6 substrate in a complex. Alternatively, TWIK-6 could be coupled with a radioisotope or enzymatic label to monitor the ability of a test compound to modulate TWIK-6 binding to a TWIK-6 substrate in a complex. Determining the ability of the test compound to bind TWIK-6 can be accomplished, for example, by coupling the compound with a radioisotope or enzymatic label such that binding of the compound to TWIK-6 can be determined by detecting the labeled TWIK-6 compound in a complex. For example, compounds (e.g., TWIK-6 substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a TWIK-6 substrate) to interact with TWIK-6 without the labeling of any of the interactants. For example, a microphysiometer can be used to detect the interaction of a compound with TWIK-6 without the labeling of either the compound or the TWIK-6. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and TWIK-6.

In another embodiment, an assay is a cell-based assay comprising contacting a cell expressing a TWIK-6 target molecule (e.g., a TWIK-6 substrate) with a test compound and determining the ability of the test compound to modulate (e.g., stimulate or inhibit) the activity of the TWIK-6 target molecule. Determining the ability of the test compound to modulate the activity of a TWIK-6 target molecule can be accomplished, for example, by determining the ability of the TWIK-6 protein to bind to or interact with the TWIK-6 target molecule.

Determining the ability of the TWIK-6 protein, or a biologically active fragment thereof, to bind to or interact with a TWIK-6 target molecule can be accomplished by one of the methods described above for determining direct binding. In a preferred embodiment, determining the ability of the TWIK-6 protein to bind to or interact with a TWIK-6 target molecule can be accomplished by determining the activity of the target molecule. For example, the activity of the target molecule can be determined by detecting induction of a cellular response (i.e., changes in intracellular K⁺ levels), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which a TWIK-6 protein or biologically active portion thereof is contacted with a test compound and the ability of the test compound to bind to the TWIK-6 protein or biologically active portion thereof is determined. Preferred biologically active portions of the TWIK-6 proteins to be used in assays of the present invention include fragments which participate in interactions with non-TWIK-6 molecules, e.g., fragments with high surface probability scores (see, for example, FIG. 3). Binding of the test compound to the TWIK-6 protein can be determined either directly or indirectly as described above. In a preferred embodiment, the assay includes contacting the TWIK-6 protein or biologically active portion thereof with a known compound which binds TWIK-6 to form an assay mixture, contacting the assay mixture with a test compound, and determining the ability of the test compound to interact with a TWIK-6 protein, wherein determining the ability of the test compound to interact with a TWIK-6 protein comprises determining the ability of the test compound to preferentially bind to TWIK-6 or biologically active portion thereof as compared to the known compound.

In another embodiment, the assay is a cell-free assay in which a TWIK-6 protein or biologically active portion thereof is contacted with a test compound and the ability of the test compound to modulate (e.g., stimulate or inhibit) the activity of the TWIK-6 protein or biologically active portion thereof is determined. Determining the ability of the test compound to modulate the activity of a TWIK-6 protein can be accomplished, for example, by determining the ability of the TWIK-6 protein to bind to a TWIK-6 target molecule by one of the methods described above for determining direct binding. Determining the ability of the TWIK-6 protein to bind to a TWIK-6 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a TWIK-6 protein can be accomplished by determining the ability of the TWIK-6 protein to further modulate the activity of a downstream effector of a TWIK-6 target molecule. For example, the activity of the effector molecule on an appropriate target can be determined or the binding of the effector to an appropriate target can be determined as previously described.

In yet another embodiment, the cell-free assay involves contacting a TWIK-6 protein or biologically active portion thereof with a known compound which binds the TWIK-6 protein to form an assay mixture, contacting the assay mixture with a test compound, and determining the ability of the test compound to interact with the TWIK-6 protein, wherein determining the ability of the test compound to interact with the TWIK-6 protein comprises determining the ability of the TWIK-6protein to preferentially bind to or modulate the activity of a TWIK-6 target molecule.

In more than one embodiment of the above assay methods of the present invention, it may be desirable to immobilize either TWIK-6 or its target molecule to facilitate separation of complexed from uncomplexed forms of one or both of the proteins, as well as to accommodate automation of the assay. Binding of a test compound to a TWIK-6 protein, or interaction of a TWIK-6 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/TWIK-6 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or TWIK-6 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of TWIK-6 binding or activity determined using standard techniques.

Other techniques for immobilizing proteins on matrices can also be used in the screening assays of the invention. For example, either a TWIK-6 protein or a TWIK-6 target molecule can be immobilized utilizing conjugation of biotin and streptavidin. Biotinylated TWIK-6 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with TWIK-6 protein or target molecules but which do not interfere with binding of the TWIK-6 protein to its target molecule can be derivatized to the wells of the plate, and unbound target or TWIK-6 protein trapped in the wells by antibody conjugation. Methods for detecting such complexes, in addition to those described above for the GST-immobilized complexes, include immunodetection of complexes using antibodies reactive with the TWIK-6 protein or target molecule, as well as enzyme-linked assays which rely on detecting an enzymatic activity associated with the TWIK-6 protein or target molecule.

In another embodiment, modulators of TWIK-6 expression are identified in a method wherein a cell is contacted with a candidate compound and the expression of TWIK-6 mRNA or protein in the cell is determined. The level of expression of TWIK-6 mRNA or protein in the presence of the candidate compound is compared to the level of expression of TWIK-6 mRNA or protein in the absence of the candidate compound. The candidate compound can then be identified as a modulator of TWIK-6 expression based on this comparison. For example, when expression of TWIK-6 mRNA or protein is greater (statistically significantly greater) in the presence of the candidate compound than in its absence, the candidate compound is identified as a stimulator of TWIK-6 mRNA or protein expression. Alternatively, when expression of TWIK-6 mRNA or protein is less (statistically significantly less) in the presence of the candidate compound than in its absence, the candidate compound is identified as an inhibitor of TWIK-6 mRNA or protein expression. The level of TWIK-6 mRNA or protein expression in the cells can be determined by methods described herein for detecting TWIK-6 mRNA or protein.

In yet another aspect of the invention, the TWIK-6 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with TWIK-6 (“TWIK-6-binding proteins” or “TWIK-6-bp”) and are involved in TWIK-6 activity. Such TWIK-6-binding proteins are also likely to be involved in the propagation of signals by the TWIK-6 proteins or TWIK-6 targets as, for example, downstream elements of a TWIK-6-mediated signaling pathway. Alternatively, such TWIK-6-binding proteins are likely to be TWIK-6 inhibitors.

The two-hybrid system is based on the modular nature of most transcription factors, which consist of separable DNA-binding and activation domains. Briefly, the assay utilizes two different DNA constructs. In one construct, the gene that codes for a TWIK-6 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TWIK-6-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the TWIK-6 protein.

In another aspect, the invention pertains to a combination of two or more of the assays described herein. For example, a modulating agent can be identified using a cell-based or a cell free assay, and the ability of the agent to modulate the activity of a TWIK-6 protein can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., a TWIK-6 modulating agent, an antisense TWIK-6 nucleic acid molecule, a TWIK-6-specific antibody, or a TWIK-6-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the TWIK-6 nucleotide sequences, described herein, can be used to map the location of the TWIK-6 genes on a chromosome. The mapping of the TWIK-6 sequences to chromosomes is an important first step in correlating these sequences with genes associated with disease.

Briefly, TWIK-6 genes can be mapped to chromosomes by preparing PCR primers (preferably 15-25 bp in length) from the TWIK-6 nucleotide sequences. Computer analysis of the TWIK-6 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the TWIK-6 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the TWIK-6 nucleotide sequences to design oligonucleotide primers, sublocalization can be achieved with panels of fragments from specific chromosomes. Other mapping strategies which can similarly be used to map a TWIK-6 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the TWIK-6 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The TWIK-6 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the TWIK-6 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The TWIK-6 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 or 4 can comfortably provide positive individual identification with a panel of perhaps 10 to 1,000 primers which each yield a noncoding amplified sequence of 100 bases. If predicted coding sequences, such as those in SEQ ID NO:3 or 6 are used, a more appropriate number of primers for positive individual identification would be 500-2,000.

If a panel of reagents from TWIK-6 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of TWIK-6 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 or 4 are particularly appropriate for this use as greater numbers of polymorphisms occur in the noncoding regions, making it easier to differentiate individuals using this technique. Examples of polynucleotide reagents include the TWIK-6 nucleotide sequences or portions thereof, e.g., fragments derived from the noncoding regions of SEQ ID NO:1 or 4 having a length of at least 20 bases, preferably at least 30 bases.

The TWIK-6 nucleotide sequences described herein can further be used to provide polynucleotide reagents, e.g., labeled or labelable probes which can be used in, for example, an in situ hybridization technique, to identify a specific tissue, e.g., thymus or brain tissue. This can be very useful in cases where a forensic pathologist is presented with a tissue of unknown origin. Panels of such TWIK-6 probes can be used to identify tissue by species and/or by organ type.

In a similar fashion, these reagents, e.g., TWIK-6 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining TWIK-6 protein and/or nucleic acid expression as well as TWIK-6 activity, in the context of a biological sample (e.g., blood, serum, cells, tissue) to thereby determine whether an individual is afflicted with a disease or disorder, or is at risk of developing a disorder, associated with aberrant or unwanted TWIK-6 expression or activity. The invention also provides for prognostic (or predictive) assays for determining whether an individual is at risk of developing a disorder associated with TWIK-6 protein, nucleic acid expression or activity. For example, mutations in a TWIK-6 gene can be assayed in a biological sample. Such assays can be used for prognostic or predictive purpose to thereby prophylactically treat an individual prior to the onset of a disorder characterized by or associated with TWIK-6 protein, nucleic acid expression or activity.

Another aspect of the invention pertains to monitoring the influence of agents (e.g., drugs, compounds) on the expression or activity of TWIK-6 in clinical trials.

These and other agents are described in further detail in the following sections.

1. Diagnostic Assays

An exemplary method for detecting the presence or absence of TWIK-6 protein or nucleic acid in a biological sample involves obtaining a biological sample from a test subject and contacting the biological sample with a compound or an agent capable of detecting TWIK-6 protein or nucleic acid (e.g., mRNA, or genomic DNA) that encodes TWIK-6 protein such that the presence of TWIK-6 protein or nucleic acid is detected in the biological sample. A preferred agent for detecting TWIK-6 mRNA or genomic DNA is a labeled nucleic acid probe capable of hybridizing to TWIK-6 mRNA or genomic DNA. The nucleic acid probe can be, for example, the TWIK-6 nucleic acid set forth in SEQ ID NO:1 or 3, or SEQ ID NO:4 or 6, or the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to TWIK-6 mRNA or genomic DNA. Other suitable probes for use in the diagnostic assays of the invention are described herein.

A preferred agent for detecting TWIK-6 protein is an antibody capable of binding to TWIK-6 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect TWIK-6 mRNA, protein, or genomic DNA in a biological sample in vitro as well as in vivo. For example, in vitro techniques for detection of TWIK-6 mRNA include Northern hybridizations and in situ hybridizations. In vitro techniques for detection of TWIK-6 protein include enzyme linked immunosorbent assays (ELISAs), Western blots, immunoprecipitations and immunofluorescence. In vitro techniques for detection of TWIK-6 genomic DNA include Southern hybridizations. Furthermore, in vivo techniques for detection of TWIK-6 protein include introducing into a subject a labeled anti-TWIK-6 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting TWIK-6 protein, mRNA, or genomic DNA, such that the presence of TWIK-6 protein, mRNA or genomic DNA is detected in the biological sample, and comparing the presence of TWIK-6 protein, mRNA or genomic DNA in the control sample with the presence of TWIK-6 protein, mRNA or genomic DNA in the test sample.

The invention also encompasses kits for detecting the presence of TWIK-6 in a biological sample. For example, the kit can comprise a labeled compound or agent capable of detecting TWIK-6 protein or mRNA in a biological sample; means for determining the amount of TWIK-6 in the sample; and means for comparing the amount of TWIK-6 in the sample with a standard. The compound or agent can be packaged in a suitable container. The kit can further comprise instructions for using the kit to detect TWIK-6 protein or nucleic acid.

2. Prognostic Assays

The diagnostic methods described herein can furthermore be utilized to identify subjects having or at risk of developing a disease or disorder associated with aberrant or unwanted TWIK-6 expression or activity. As used herein, the term “aberrant” includes a TWIK-6 expression or activity which deviates from the wild type TWIK-6 expression or activity. Aberrant expression or activity includes increased or decreased expression or activity, as well as expression or activity which does not follow the wild type developmental pattern of expression or the subcellular pattern of expression. For example, aberrant TWIK-6 expression or activity is intended to include the cases in which a mutation in the TWIK-6 gene causes the TWIK-6 gene to be under-expressed or over-expressed and situations in which such mutations result in a non-functional TWIK-6 protein or a protein which does not function in a wild-type fashion, e.g., a protein which does not interact with a TWIK-6 substrate, e.g., a non-potassium channel subunit or ligand, or one which interacts with a non-TWIK-6 substrate, e.g. a non-potassium channel subunit or ligand. As used herein, the term “unwanted” includes an unwanted phenomenon involved in a biological response such as cellular proliferation. For example, the term unwanted includes a TWIK-6 expression or activity which is undesirable in a subject.

The assays described herein, such as the preceding diagnostic assays or the following assays, can be utilized to identify a subject having or at risk of developing a disorder associated with a misregulation in TWIK-6 protein activity or nucleic acid expression, such as a CNS disorder (e.g., a cognitive or neurodegenerative disorder), a cellular proliferation, growth, differentiation, or migration disorder, or a cardiovascular disorder. Alternatively, the prognostic assays can be utilized to identify a subject having or at risk for developing a disorder associated with a misregulation in TWIK-6 protein activity or nucleic acid expression, such as a CNS disorder, a cellular proliferation, growth, differentiation, or migration disorder, or a cardiovascular disorder. Thus, the present invention provides a method for identifying a disease or disorder associated with aberrant or unwanted TWIK-6 expression or activity in which a test sample is obtained from a subject and TWIK-6 protein or nucleic acid (e.g., mRNA or genomic DNA) is detected, wherein the presence of TWIK-6 protein or nucleic acid is diagnostic for a subject having or at risk of developing a disease or disorder associated with aberrant or unwanted TWIK-6 expression or activity. As used herein, a “test sample” refers to a biological sample obtained from a subject of interest. For example, a test sample can be a biological fluid (e.g., cerebrospinal fluid or serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted TWIK-6 expression or activity. For example, such methods can be used to determine whether a subject can be effectively treated with an agent for a CNS disorder, a muscular disorder, or a cellular proliferation, growth, differentiation, or migration disorder. Thus, the present invention provides methods for determining whether a subject can be effectively treated with an agent for a disorder associated with aberrant or unwanted TWIK-6 expression or activity in which a test sample is obtained and TWIK-6 protein or nucleic acid expression or activity is detected (e.g., wherein the abundance of TWIK-6 protein or nucleic acid expression or activity is diagnostic for a subject that can be administered the agent to treat a disorder associated with aberrant or unwanted TWIK-6 expression or activity).

The methods of the invention can also be used to detect genetic alterations in a TWIK-6 gene, thereby determining if a subject with the altered gene is at risk for a disorder characterized by misregulation in TWIK-6 protein activity or nucleic acid expression, such as a CNS disorder, a cellular proliferation, growth, differentiation, or migration disorder, or cardiovascular disorder. In preferred embodiments, the methods include detecting, in a sample of cells from the subject, the presence or absence of a genetic alteration characterized by at least one of an alteration affecting the integrity of a gene encoding a TWIK-6-protein, or the mis-expression of the TWIK-6 gene. For example, such genetic alterations can be detected by ascertaining the existence of at least one of 1) a deletion of one or more nucleotides from a TWIK-6 gene; 2) an addition of one or more nucleotides to a TWIK-6 gene; 3) a substitution of one or more nucleotides of a TWIK-6 gene, 4) a chromosomal rearrangement of a TWIK-6 gene; 5) an alteration in the level of a messenger RNA transcript of a TWIK-6 gene, 6) aberrant modification of a TWIK-6 gene, such as of the methylation pattern of the genomic DNA, 7) the presence of a non-wild type splicing pattern of a messenger RNA transcript of a TWIK-6 gene, 8) a non-wild type level of a TWIK-6-protein, 9) allelic loss of a TWIK-6 gene, and 10) inappropriate post-translational modification of a TWIK-6-protein. As described herein, there are a large number of assays known in the art which can be used for detecting alterations in a TWIK-6 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the TWIK-6-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a TWIK-6 gene under conditions such that hybridization and amplification of the TWIK-6-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P.M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a TWIK-6 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in TWIK-6 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in TWIK-6 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the TWIK-6 gene and detect mutations by comparing the sequence of the sample TWIK-6 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g, PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the TWIK-6 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type TWIK-6 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in TWIK-6 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a TWIK-6 sequence, e.g., a wild-type TWIK-6 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in TWIK-6 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control TWIK-6 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a TWIK-6 gene.

Furthermore, any cell type or tissue in which TWIK-6 is expressed may be utilized in the prognostic assays described herein.

3. Monitoring of Effects During Clinical Trials

Monitoring the influence of agents (e.g., drugs) on the expression or activity of a TWIK-6 protein (e.g., the modulation of cell proliferation and/or migration) can be applied not only in basic drug screening, but also in clinical trials. For example, the effectiveness of an agent determined by a screening assay as described herein to increase TWIK-6 gene expression, protein levels, or upregulate TWIK-6 activity, can be monitored in clinical trials of subjects exhibiting decreased TWIK-6 gene expression, protein levels, or downregulated TWIK-6 activity. Alternatively, the effectiveness of an agent determined by a screening assay to decrease TWIK-6 gene expression, protein levels, or downregulate TWIK-6 activity, can be monitored in clinical trials of subjects exhibiting increased TWIK-6 gene expression, protein levels, or upregulated TWIK-6 activity. In such clinical trials, the expression or activity of a TWIK-6 gene, and preferably, other genes that have been implicated in, for example, a TWIK-6-associated disorder can be used as a “read out” or markers of the phenotype of a particular cell.

For example, and not by way of limitation, genes, including TWIK-6, that are modulated in cells by treatment with an agent (e.g., compound, drug or small molecule) which modulates TWIK-6 activity (e.g., identified in a screening assay as described herein) can be identified. Thus, to study the effect of agents on TWIK-6-associated disorders (e.g., disorders characterized by deregulated cell proliferation and/or migration), for example, in a clinical trial, cells can be isolated and RNA prepared and analyzed for the levels of expression of TWIK-6 and other genes implicated in the TWIK-6-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of TWIK-6 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a TWIK-6 protein, mRNA, or genomic DNA in the preadministration sample; (iii) obtaining one or more post-administration samples from the subject; (iv) detecting the level of expression or activity of the TWIK-6 protein, mRNA, or genomic DNA in the post-administration samples; (v) comparing the level of expression or activity of the TWIK-6 protein, mRNA, or genomic DNA in the pre-administration sample with the TWIK-6 protein, mRNA, or genomic DNA in the post administration sample or samples; and (vi) altering the administration of the agent to the subject accordingly. For example, increased administration of the agent may be desirable to increase the expression or activity of TWIK-6 to higher levels than detected, i.e., to increase the effectiveness of the agent. Alternatively, decreased administration of the agent may be desirable to decrease expression or activity of TWIK-6 to lower levels than detected, i.e. to decrease the effectiveness of the agent. According to such an embodiment, TWIK-6 expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted TWIK-6 expression or activity, e.g. a CNS disorder, a cellular proliferation, growth, differentiation, or migration disorder, or a cardiovascular disorder. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the TWIK-6 molecules of the present invention or TWIK-6 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted TWIK-6 expression or activity, by administering to the subject a TWIK-6 or an agent which modulates TWIK-6 expression or at least one TWIK-6 activity. Subjects at risk for a disease which is caused or contributed to by aberrant or unwanted TWIK-6 expression or activity can be identified by, for example, any or a combination of diagnostic or prognostic assays as described herein. Administration of a prophylactic agent can occur prior to the manifestation of symptoms characteristic of the TWIK-6 aberrancy, such that a disease or disorder is prevented or, alternatively, delayed in its progression. Depending on the type of TWIK-6 aberrancy, for example, a TWIK-6, TWIK-6 agonist or TWIK-6 antagonist agent can be used for treating the subject. The appropriate agent can be determined based on screening assays described herein.

2. Therapeutic Methods

Another aspect of the invention pertains to methods of modulating TWIK-6 expression or activity for therapeutic purposes. Accordingly, in an exemplary embodiment, the modulatory method of the invention involves contacting a cell with a TWIK-6 or agent that modulates one or more of the activities of TWIK-6 protein activity associated with the cell. An agent that modulates TWIK-6 protein activity can be an agent as described herein, such as a nucleic acid or a protein, a naturally-occurring target molecule of a TWIK-6 protein (e.g., a TWIK-6 substrate), a TWIK-6 antibody, a TWIK-6 agonist or antagonist, a peptidomimetic of a TWIK-6 agonist or antagonist, or other small molecule. In one embodiment, the agent stimulates one or more TWIK-6 activities. Examples of such stimulatory agents include active TWIK-6 protein and a nucleic acid molecule encoding TWIK-6 that has been introduced into the cell. In another embodiment, the agent inhibits one or more TWIK-6 activities. Examples of such inhibitory agents include antisense TWIK-6 nucleic acid molecules, anti-TWIK-6 antibodies, and TWIK-6 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a TWIK-6 protein or nucleic acid molecule. In one embodiment, the method involves administering an agent (e.g., an agent identified by a screening assay described herein), or combination of agents that modulates (e.g., upregulates or downregulates) TWIK-6 expression or activity. In another embodiment, the method involves administering a TWIK-6 protein or nucleic acid molecule as therapy to compensate for reduced, aberrant, or unwanted TWIK-6 expression or activity.

Stimulation of TWIK-6 activity is desirable in situations in which TWIK-6 is abnormally downregulated and/or in which increased TWIK-6 activity is likely to have a beneficial effect. Likewise, inhibition of TWIK-6 activity is desirable in situations in which TWIK-6 is abnormally upregulated and/or in which decreased TWIK-6 activity is likely to have a beneficial effect.

3. Pharmacogenomics

The TWIK-6 molecules of the present invention, as well as agents, or modulators which have a stimulatory or inhibitory effect on TWIK-6 activity (e.g., TWIK-6 gene expression) as identified by a screening assay described herein can be administered to individuals to treat (prophylactically or therapeutically) TWIK-6-associated disorders (e.g., proliferative disorders) associated with aberrant or unwanted TWIK-6 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a TWIK-6 molecule or TWIK-6 modulator as well as tailoring the dosage and/or therapeutic regimen of treatment with a TWIK-6 molecule or TWIK-6 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a TWIK-6 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a TWIK-6 molecule or TWIK-6 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a TWIK-6 molecule or TWIK-6 modulator, such as a modulator identified by one of the exemplary screening assays described herein.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human TWIK-6 cDNA

In this example, the identification and characterization of the gene encoding human TWIK-6 (clones Fbh17831E and Fbh17831G) is described.

Isolation of the TWIK-6 cDNA

The invention is based, at least in part, on the discovery of a human gene encoding a novel protein, referred to herein as TWIK-6. The entire sequence of the human clone Fbh17831 was determined and found to contain an open reading frame termed human “TWIK-6.” It was further determined that this gene possesses an A to G nucleotide polymorphism at nucleotide 596, resulting in an E to G amino acid change at amino acid position 165 (resulting in clones Fbh17831E and Fbh17831G). The two alternate nucleotide sequences of the human TWIK-6 gene are set forth in FIGS. 1A-B and 2A-B and in the Sequence Listing as SEQ ID NOs: 1 and 3 or 4 and 6. The two alternate amino acid sequences of the human TWIK-6 expression product are set forth in FIGS. 1A-B and 2A-B and in the Sequence Listing as SEQ ID NO:2 or 5.

The nucleotide sequence encoding the human TWIK-6 (E¹⁶⁵) protein is shown in FIGS. 1A-B and is set forth as SEQ ID NO:1. The nucleotide sequence encoding the human TWIK-6 (G¹⁶⁵) protein is shown in FIGS. 2A-B and is set forth as SEQ ID NO:4. The protein encoded by each of these nucleic acids comprises about 400 amino acids and has the amino acid sequence shown in FIGS. 1A-B and 2A-B and set forth as SEQ ID NOs: 2 and 5, respectively. The coding regions (open reading frames) of SEQ ID NO:1 and 4 are set forth as SEQ ID NOs:3 and 6. Clones Fbh1783E and Fbh17831G, comprising the coding region of human TWIK-6 were deposited with the American Type Culture Collection (ATCC®), 10801 University Boulevard, Manassas, Va. 20110-2209, on ______, and assigned Accession No ______.

Analysis of the Human TWIK-6 Molecules

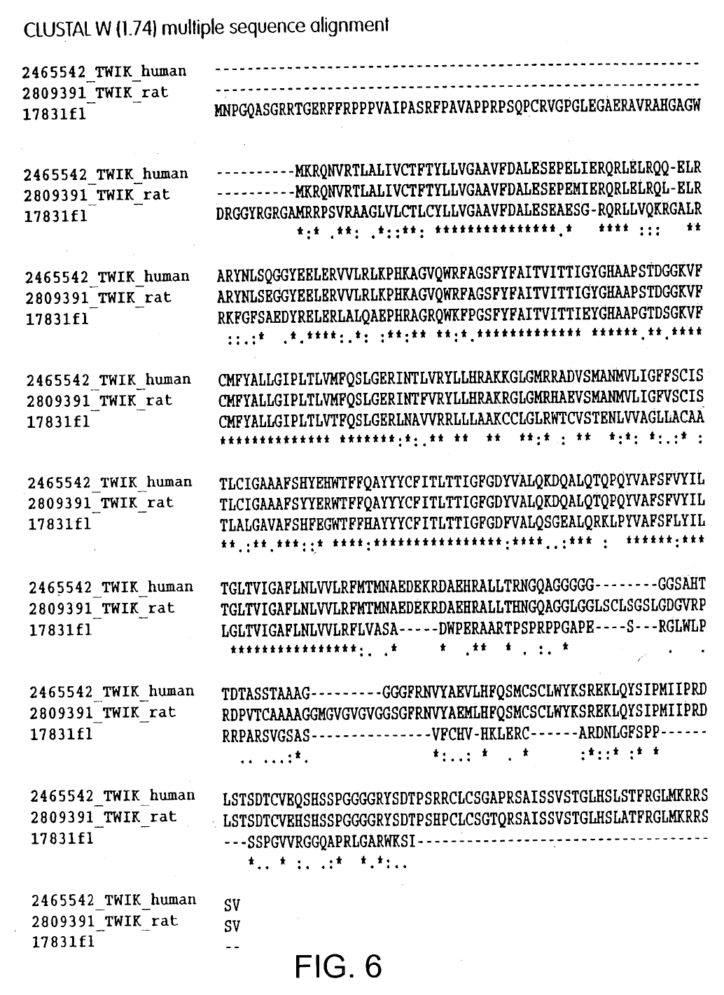

A BLASTN 2.0 search against the NRN database, using a score of 100 and a wordlength of 12 (Altschul et al. (1990) J. Mol. Biol. 215:403) of the nucleotide sequence of human TWIK-6 revealed that human TWIK-6 is 72% identical to Homo sapiens TWIK-related acid-sensitive K+ channel (TASK) mRNA (Accession Number AF006823) over nucleotides 200-1158 (as shown in FIG. 6). The search further revealed that human TWIK-6 is 73% identical to Mus musculus mRNA for cTBAK, complete cds (Accession Number AB013345) over nucleotides 180 to 1229. The search further revealed that human TWIK-6 is 69% identical to Rattus norvegicus TWIK-related acid-sensitive K+ channel (TASK) mRNA (Accession Number AF031384) over nucleotides 232 to 1229, and 78% identical over nucleotides 1482 to 1527. The search further revealed that human TWIK-6 is 70% identical to Mus musculus TWIK-related acid-sensitive K+ channel (TASK) mRNA (Accession Number AF006824) over nucleotides 324 to 1229, 77% identical over nucleotides 1492 to 1526, 95% identical over nucleotides 1506 to 1527, and 95% identical over nucleotides 1507 to 1528. The search further revealed that human TWIK-6 is 70% identical to Mus musculus mRNA for cTBAK (Accession Number AB008537) over nucleotides 303 to 924. This search further revealed that human TWIK-6 is 71% identical to Mus musculus putative potassium channel DP4 mRNA (Accession Number AF022821) over nucleotides 318 to 1217. This search further revealed that human TWIK-6 is homologous to genomic sequence for Homo sapiens clone 431C18 from chromosome 8 (locus D8S1741) (Accession Number AC007869)

A BLASTN 2.0 search against the dbEST database, using a score of 100 and a wordlength of 12 (Altschul et al. (1990) J. Mol. Biol. 215:403) of the nucleotide sequence of human TWIK-6 revealed that human TWIK-6 is 97% identical to xg70f06.x1 NCI_CGAP_Ut4 Homo sapiens cDNA clone IMAGE:2633699 3′ similar to TR:o35111 O35111 CTBAK (Accession Number AW167075) over nucleotides 1496 to 895. The search further revealed that human TWIK-6 is 98% identical to tz15g05.x1 NCI_CGAP_Ut2 Homo sapiens cDNA clone IMAGE:2288696 3′ similar to TR:O35111 O35111 CTBAK (Accession Number AI690321) over nucleotides 415 to 871. This search further revealed that human TWIK-6 is 100% identical to wi17h03.x1NCI_CGAP_Co16 Homo sapiens cDNA clone IMAGE: 2390549 3′ similar to TR:O35111 O35111 CTBAK (Accession Number AI739096) over nucleotides 460-827. This search further revealed that human TWIK-6 is 73% identical to ma07h04.y1 Soares mouse p3NMF19.5 Mus musculus cDNA clone IMAGE: 303895 5′ similar to TR:O35163 O35163 TWIK-RELATED ACID-SENSITIVE K+ CHANNEL (Accession Number AI605559) over nucleotides 324-998. The search further revealed that human TWIK-6 is 97% identical to oo19e06.x1 Soares_NSF_F8_{—}9W_OT₁₃ PA_P_S1 Homo sapiens cDNA clone IMAGE:1566658 (Accession Number AI091631) over nucleotides 1528 to 1193. This search further revealed that human TWIK-6 is 89% identical to wt90e12.x1 NCI_CGAP_GC6 Homo sapiens cDNA clone IMAGE: 2514766 3′ similar to TR:014649 O14649 TWIK-RELATED ACID-SENSITIVE K+ CHANNEL (Accession Number AI968607) over nucleotides 417-808 and 92% identical over nucleotides 388 to 219. The search further revealed that human TWIK-6 is 72% identical to mf89d07.x1 Soares mouse embryo NbME13.5 14.5 Mus musculus cDNA clone IMAGE:421453 3′ similar to WP:C40C9.1 CE05398 POTASSIUM CHANNEL PROTEIN (Accession Number AI327069) over nucleotides 1173 to 553. The search further revealed that human TWIK-6 is 76% identical to mf89d07.y1 Soares mouse embryo NbME13.5 14.5 Mus musculus cDNA clone IMAGE:421453 5′ similar to TR:O35111 O35111 MCTBAK (Accession Number AI325858) over nucleotides 508 to 989. The search further revealed that human TWIK-6 is 73% identical to EST291996 normalized rat ovary, Bento Soares Rattus sp. cDNA clone RGICN44 5′ end similar to TWIK-related acid-sensitive K+ channel (Accession Number AW141881) over nucleotides 620 to 1142. The search further revealed that human TWIK-6 is 88% identical to UI-M-BH2.2-aos-c-07-0-UI.s1 NIH_BMAP_M_S3.2 Mus musculus cDNA clone UI-M-BH2.2-aos-c-07-0-UI (Accession Number AW122298) over nucleotides 764 to 1054. A BLASTN 2.0 search against the PATENT_{—}2/patentDbpreviewNuc database, using a score of 100 and a wordlength of 12 (Altschul et al. (1990) J. Mol. Biol. 215:403) of the nucleotide sequence of human TWIK-6 revealed that human TWIK-6 is 73% identical to human signal peptide-containing sequence from WO 00/00610 (Accession Number AC36763) over nucleotides 281 to 1158. This search further revealed that human TWIK-6 is 58% identical to human potassium channel genes from WO 99/43696 (Accession Number AC29586) over nucleotides 242 to 1188.

A BLASTX 2.0 search against the NRP/protot database, using a score of 100 and a wordlength of 3 (Altschul et al. (1990) J. Mol. Biol. 215:403), of the translated nucleotide sequence of human TWIK-6 revealed that human TWIK-6 is 64% identical to cTBAK from Mus musculus (Accession Number AB008537) over translated nucleotides 313 to 1086, 44% identical over translated nucleotides 1253 to 1354, and 33% identical over translated nucleotides 1253 to 1369. The search further revealed that human TWIK-6 is 65% identical to TWIK-related acid-sensitive K+ channel from Homo sapiens (Accession Number AF006823) over translated nucleotides 313 to 1086, 40% identical over translated nucleotides 1265 to 1354, and 33% identical over translated nucleotides 1253 to 1369 (as shown in FIG. 6). The search further revealed that human TWIK-6 is 64% identical to TWIK-related acid-sensitive K+ channel from Rattus norvegicus (Accession Number AF031384) over translated nucleotides 313 to 1086, 42% identical over translated nucleotides 1253 to 1354, and 33% identical over translated nucleotides 1253 to 1369. The search further revealed that human TWIK-6 is 64% identical to TWIK-related acid-sensitive K+ channel from Mus musculus (Accession Number AF006824) over translated nucleotides 325 to 1086, 44% identical over translated nucleotides 1253 to 1354, and 33% identical over translated nucleotides 1253 to 1369. The search further revealed that human TWIK-6 is 65% identical to putative potassium channel DP4 from Mus musculus (Accession Number AAD09338) over translated nucleotides 325 to 1086. The search further revealed that human TWIK-6 is 52% identical to putative potassium channel subunit n2P38 from Caenorhabditis elegans (Accession Number AF083652) over translated nucleotides 313 to 1068. The search further revealed that human TWIK-6 is 51% identical to outward rectifier potassium channel homolog twk-23 from Caenorhabditis elegans (Accession Number AF025454) over translated nucleotides 313 to 1068.

An alignment of the human TWIK-6 amino acid sequence with the amino acid sequences of TWIK-related acid-sensitive K+ channel from Homo sapiens (Accession Number AF006823) and TWIK-related acid-sensitive K+ channel from Rattus norvegicus (Accession Number AF031384) using the CLUSTAL W (1.74) multiple sequence alignment program is set forth in FIG. 6.

A search was performed against the Memsat database (FIG. 6), resulting in the identification of four transmembrane domains in the amino acid sequence of human TWIK-6 (SEQ ID NO: 2 or SEQ ID NO:4) at about residues 78-96, 178-195, 229-248, and 290-314. This search further identified two Pore-loop domains (P-loop domains) at residues 150-166 and 260-278.

A search was also performed against the ProDom database resulting in the identification of a potassium channel protein domain in the amino acid sequence of human TWIK-6 (SEQ ID NO:2 or 5) at about residues 81-312 (score=162). This search further resulted in the identification of a TWIK-related ion channel domain in the amino acid sequence of human TWIK-6 (SEQ ID NO:2 or 5) at about residues 75-144 (score=149). The results of the search are set forth in FIG. 8.

Tissue Distribution of TWIK-6 mRNA

This example describes the tissue distribution of TWIK-6 mRNA, as was determined by Polymerase Chain Reaction (PCR) on cDNA libraries using oligonucleotide primers based on the human TWIK-6 sequence.

The human TWIK-6 gene is expressed in thymus, in salivary gland, in primary cultured osteoblasts, and in ovarian epithelium tumor cells.

### Example 2

### Expression of Recombinant TWIK-6 Protein in Bacterial Cells

In this example, TWIK-6 is expressed as a recombinant glutathione-S-transferase (GST) fusion polypeptide in E. coli and the fusion polypeptide is isolated and characterized. Specifically, TWIK-6 is fused to GST and this fusion polypeptide is expressed in E. coli, e.g., strain PEB199. Expression of the GST-TWIK-6 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant TWIK-6 Protein in COS Cells

To express the TWIK-6 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire TWIK-6 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the TWIK-6 DNA sequence is amplified by PCR using two primers. The 5′ primer contains the restriction site of interest followed by approximately twenty nucleotides of the TWIK-6 coding sequence starting from the initiation codon; the 3′ end sequence contains complementary sequences to the other restriction site of interest, a translation stop codon, the HA tag or FLAG tag and the last 20 nucleotides of the TWIK-6 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the TWIK-6 gene is inserted in the correct orientation. The ligation mixture is transformed into E. coil cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the TWIK-6-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the TWIK-6 polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the TWIK-6 coding sequence is cloned directly into the polylinker of the pCDNA/Amp vector using the appropriate restriction sites. The resulting plasmid is transfected into COS cells in the manner described above, and the expression of the TWIK-6 polypeptide is detected by radiolabeling and immunoprecipitation using a TWIK-6 specific monoclonal antibody.

### II. NUCLEIC ACIDS ENCODING TWIK-7, A NOVEL POTASSIUM CHANNEL PROTEIN

### Background of the Invention

Potassium (K⁺) channels are ubiquitous proteins which are involved in the setting of the resting membrane potential as well as in the modulation of the electrical activity of cells. In excitable cells, K⁺ channels influence action potential waveforms, firing frequency, and neurotransmitter secretion (Rudy, B. (1988) Neuroscience, 25, 729-749; Hille, B. (1992) Ionic Channels of Excitable Membranes, 2nd Ed.). In non-excitable cells, they are involved in hormone secretion, cell volume regulation and potentially in cell proliferation and differentiation (Lewis et al. (1995) Annu. Rev. Immunol., 13, 623-653). Developments in electrophysiology have allowed the identification and the characterization of an astonishing variety of K⁺ channels that differ in their biophysical properties, pharmacology, regulation and tissue distribution (Rudy, B. (1988) Neuroscience, 25, 729-749; Hille, B. (1992) Ionic Channels of Excitable Membranes, 2nd Ed.). More recently, cloning efforts have shed considerable light on the mechanisms that determine this functional diversity. Furthermore, analyses of structure-function relationships have provided an important set of data concerning the molecular basis of the biophysical properties (selectivity, gating, assembly) and the pharmacological properties of cloned K⁺ channels.

Functional diversity of K⁺ channels arises mainly from the existence of a great number of genes coding for pore-forming subunits, as well as for other associated regulatory subunits. Two main structural families of pore-forming subunits have been identified. The first one consists of subunits with a conserved hydrophobic core containing six transmembrane domains (TMDs). These K⁺ channel a subunits participate in the formation of outward rectifier voltage-gated (Kv) and Ca²⁺-dependent K⁺ channels. The fourth TMD contains repeated positive charges involved in the voltage gating of these channels and hence in their outward rectification (Logothetis et al. (1992) Neuron, 8, 531-540; Bezanilla et al. (1994) Biophys. J. 66,1011-1021).

The second family of pore-forming subunits have only two TMDs. They are essential subunits of inward-rectifying (IRK), G-protein-coupled (GIRK) and ATP-sensitive (K_{ATP}) K⁺ channels. The inward rectification results from a voltage-dependent block by cytoplasmic Mg²⁺ and polyamines (Matsuda, H. (1991) Annu. Rev. Physiol., 53, 289-298). A conserved domain, called the P domain, is present in all members of both families (Pongs, O. (1993) J. Membr. Biol., 136, 1-8; Heginbotham et al. (1994) Biophys. J. 66,1061-1067; Mackinnon, R. (1995) Neuron, 14, 889-892; Pascual et al., (1995) Neuron., and 14, 1055-1063). This domain is an essential element of the aqueous K⁺-selective pore. In both groups, the assembly of four subunits is necessary to form a functional K⁺ channel (Mackinnon, R. (1991) Nature, 350, 232-235; Yang et al., (1995) Neuron, 15, 1441-1447.

In both six TMD and two TMD pore-forming subunit families, different subunits coded by different genes can associate to form heterotetramers with new channel properties (Isacoff et al., (1990) Nature, 345, 530-534). A selective formation of heteropolymeric channels may allow each cell to develop the best K⁺ current repertoire suited to its function. Pore-forming α subunits of Kv channels are classified into different subfamilies according to their sequence similarity (Chandy et al. (1993) Trends Pharmacol Sci., 14, 434). Tetramerization is believed to occur preferentially between members of each subgroup (Covarrubias et al. (1991) Neuron, 7, 763-773). The domain responsible for this selective association is localized in the N-terminal region and is conserved between members of the same subgroup. This domain is necessary for hetero- but not homomultimeric assembly within a subfamily and prevents co-assembly between subfamilies. Recently, pore-forming subunits with two TMDs were also shown to co-assemble to form heteropolymers (Duprat et al. (1995) Biochem. Biophys. Res. Commun., 212, 657-663. This heteropolymerization seems necessary to give functional GIRKs. IRKs are active as homopolymers but also form heteropolymers.

New structural types of K⁺ channels were identified recently in both humans and yeast. These channels have two P domains in their functional subunit instead of only one (Ketchum et al. (1995) Nature, 376, 690-695; Lesage et al. (1996) J. Biol. Chem, 271, 4183-4187; Lesage et al. (1996) EMBO J., 15, 1004-1011; Reid et al. (1996) Receptors Channels 4, 51-62). The human channel called TWIK-1, has four TMDs. TWIK-1 is expressed widely in human tissues and is particularly abundant in the heart and the brain. TWIK-1 currents are time independent and inwardly rectifying. These properties suggest that TWIK-1 channels are involved in the control of the background K⁺ membrane conductance (Lesage et al. (1996) EMBO J., 15, 1004-1011).

### Summary of the Invention

The present invention is based, at least in part, on the discovery of novel members of the TWIK (for Tandem of P domains in a Weak Inward rectifying K⁺ channel) family of potassium channels, referred to herein as TWIK-7 nucleic acid and protein molecules. The TWIK-7 molecules of the present invention are useful as targets for developing modulating agents to regulate a variety of cellular processes. Accordingly, in one aspect, this invention provides isolated nucleic acid molecules encoding TWIK proteins or biologically active portions thereof, as well as nucleic acid fragments suitable as primers or hybridization probes for the detection of TWIK-encoding nucleic acids.

In one embodiment, a TWIK-7 nucleic acid molecule of the invention is at least 50%, 55%, 60%, 65%, 70%, 73%, 74%, 75%, 80%, 85%, 90%, 95%, 96%, 97%, 98%, 99% or more identical to the nucleotide sequence (e.g., to the entire length of the nucleotide sequence) shown in SEQ ID NO:14 or 16, or a complement thereof. In a preferred embodiment, the isolated nucleic acid molecule includes the nucleotide sequence shown SEQ ID NO:14 or 16, or a complement thereof. In another embodiment, the nucleic acid molecule includes SEQ ID NO:16 and nucleotides 1-465 of SEQ ID NO:14. In yet another embodiment, the nucleic acid molecule includes SEQ ID NO:16 and nucleotides 1759-1943 of SEQ ID NO:14. In another preferred embodiment, the nucleic acid molecule consists of the nucleotide sequence shown in SEQ ID NO:14 or 16. In another preferred embodiment, the nucleic acid molecule includes a fragment of at least 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, or 1910 nucleotides (e.g., 100 contiguous nucleotides) of the nucleotide sequence of SEQ ID NO:14 or 16, or a complement thereof.

In another embodiment, a TWIK-7 nucleic acid molecule includes a nucleotide sequence encoding a protein having an amino acid sequence sufficiently identical to the amino acid sequence of SEQ ID NO:15. In a preferred embodiment, a TWIK-7 nucleic acid molecule includes a nucleotide sequence encoding a protein having an amino acid sequence at least 50%, 55%, 60%, 65%, 67%, 68%, 70%, 75%, 80%, 85%, 90%, 95%, 96%, 97%, 98%, 99% or more identical to the entire length of the amino acid sequence of SEQ ID NO:15.

In another preferred embodiment, an isolated nucleic acid molecule encodes the amino acid sequence of human TWIK-7. In yet another preferred embodiment, the nucleic acid molecule includes a nucleotide sequence encoding a protein having the amino acid sequence of SEQ ID NO:15. In yet another preferred embodiment, the nucleic acid molecule is at least 1910 nucleotides in length. In a further preferred embodiment, the nucleic acid molecule is at least 1910 nucleotides in length and encodes a protein having a TWIK-7 activity (as described herein).

Another embodiment of the invention features nucleic acid molecules, preferably TWIK-7 nucleic acid molecules, which specifically detect TWIK-7 nucleic acid molecules relative to nucleic acid molecules encoding non-TWIK-7 proteins. For example, in one embodiment, such a nucleic acid molecule is at least 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1910, 1910-1920, 1920-1940, or more nucleotides in length and hybridizes under stringent conditions to a nucleic acid molecule comprising the nucleotide sequence shown in SEQ ID NO:14 or a complement thereof.

In other preferred embodiments, the nucleic acid molecule encodes a naturally occurring allelic variant of a polypeptide comprising the amino acid sequence of SEQ ID NO:15, wherein the nucleic acid molecule hybridizes to a nucleic acid molecule comprising SEQ ID NO:14 or 16 under stringent conditions.

Another embodiment of the invention provides an isolated nucleic acid molecule which is antisense to a TWIK-7 nucleic acid molecule, e.g., the coding strand of a TWIK-7 nucleic acid molecule.

Another aspect of the invention provides a vector comprising a TWIK-7 nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably a TWIK-7 protein, by culturing in a suitable medium, a host cell, e.g., a mammalian host cell such as a non-human mammalian cell, of the invention containing a recombinant expression vector, such that the protein is produced.

Another aspect of this invention features isolated or recombinant TWIK-7 proteins and polypeptides. In one embodiment, an isolated TWIK-7 protein includes at least one transmembrane domain. In yet another embodiment, an isolated TWIK-7 protein includes a potassium channel protein domain. In yet another embodiment, an isolated TWIK-7 protein includes a P-loop domain. In yet another embodiment, an isolated TWIK-7 protein includes at least one transmembrane domain and one or more of a P-loop domain and a potassium channel protein domain. In yet another embodiment, an isolated TWIK-7 protein includes at least one transmembrane domain, a P-loop domain, and a potassium channel protein domain.

In a preferred embodiment, a TWIK-7 protein includes at least one transmembrane domain and has an amino acid sequence at least about 50%, 55%, 60%, 65%, 67%, 68%, 70%, 75%, 80%, 85%, 90%, 95%, 96%, 97%, 98%, 99% or more identical to the amino acid sequence of SEQ ID NO:15. In a further preferred embodiment, a TWIK-7 protein includes a potassium channel protein domain and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:15. In a further preferred embodiment, a TWIK-7 protein includes at least one P-loop domain and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:15. In a further preferred embodiment, a TWIK-7 protein includes at least one transmembrane domain and one or more of a P-loop domain and/or a potassium channel protein domain, and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:15.

In another preferred embodiment, a TWIK-7 protein includes at least one transmembrane domain and has a TWIK-7 activity (as described herein).

In yet another preferred embodiment, a TWIK-7 protein includes at least one transmembrane domain and is encoded by a nucleic acid molecule having a nucleotide sequence which hybridizes under stringent hybridization conditions to a nucleic acid molecule comprising the nucleotide sequence of SEQ ID NO:14 or 16. In a further embodiment, a TWIK-7 protein includes a P-loop domain and is encoded by a nucleic acid molecule having a nucleotide sequence which hybridizes under stringent hybridization conditions to a nucleic acid molecule comprising the nucleotide sequence of SEQ ID NO:14 or 16. In a further embodiment, a TWIK-7 protein includes a potassium channel protein domain and is encoded by a nucleic acid molecule having a nucleotide sequence which hybridizes under stringent hybridization conditions to a nucleic acid molecule comprising the nucleotide sequence of SEQ ID NO:14 or 16. In another embodiment, a TWIK-7 protein includes at least one transmembrane domain and one or more of a P-loop domain and/or a potassium channel protein domain, and is encoded by a nucleic acid molecule having a nucleotide sequence which hybridizes under stringent hybridization conditions to a nucleic acid molecule comprising the nucleotide sequence of SEQ ID NO:14 or 16.

In another embodiment, the invention features fragments of the protein having the amino acid sequence of SEQ ID NO:15, wherein the fragment comprises at least 15 amino acids (e.g, 15 contiguous amino acids) of the amino acid sequence of SEQ ID NO:15. In another embodiment, a TWIK-7 protein has the amino acid sequence of SEQ ID NO:15.

In another embodiment, the invention features a TWIK-7 protein which is encoded by a nucleic acid molecule consisting of a nucleotide sequence at least about 50%, 55%, 60%, 65%, 70%, 73%, 74%, 75%, 80%, 85%, 90%, 95%, 96%, 97%, 98%, 99% or more identical to a nucleotide sequence of SEQ ID NO:14 or 16, or a complement thereof. This invention further features a TWIK-7 protein, which is encoded by a nucleic acid molecule consisting of a nucleotide sequence which hybridizes under stringent hybridization conditions to a nucleic acid molecule comprising the nucleotide sequence of SEQ ID NO:14 or 16, or a complement thereof.

The proteins of the present invention or portions thereof, e.g., biologically active portions thereof, can be operatively linked to a non-TWIK-7 polypeptide (e.g., heterologous amino acid sequences) to form fusion proteins. The invention further features antibodies, such as monoclonal or polyclonal antibodies, that specifically bind proteins of the invention, preferably TWIK-7 proteins. In addition, the TWIK-7 proteins or biologically active portions thereof can be incorporated into pharmaceutical compositions, which optionally include pharmaceutically acceptable carriers.

In another aspect, the present invention provides a method for detecting the presence of a TWIK-7 nucleic acid molecule, protein, or polypeptide in a biological sample by contacting the biological sample with an agent capable of detecting a TWIK-7 nucleic acid molecule, protein, or polypeptide such that the presence of a TWIK-7 nucleic acid molecule, protein or polypeptide is detected in the biological sample.

In another aspect, the present invention provides a method for detecting the presence of TWIK-7 activity in a biological sample by contacting the biological sample with an agent capable of detecting an indicator of TWIK-7 activity such that the presence of TWIK-7 activity is detected in the biological sample.

In another aspect, the invention provides a method for modulating TWIK-7 activity comprising contacting a cell capable of expressing TWIK-7 with an agent that modulates TWIK-7 activity such that TWIK-7 activity in the cell is modulated. In one embodiment, the agent inhibits TWIK-7 activity. In another embodiment, the agent stimulates TWIK-7 activity. In one embodiment, the agent is an antibody that specifically binds to a TWIK-7 protein. In another embodiment, the agent modulates expression of TWIK-7 by modulating transcription of a TWIK-7 gene or translation of a TWIK-7 mRNA. In yet another embodiment, the agent is a nucleic acid molecule having a nucleotide sequence that is antisense to the coding strand of a TWIK-7 mRNA or a TWIK-7 gene.

In one embodiment, the methods of the present invention are used to treat a subject having a disorder characterized by aberrant or unwanted TWIK-7 protein or nucleic acid expression or activity by administering an agent which is a TWIK-7 modulator to the subject. In one embodiment, the TWIK-7 modulator is a TWIK-7 protein. In another embodiment the TWIK-7 modulator is a TWIK-7 nucleic acid molecule. In yet another embodiment, the TWIK-7 modulator is a peptide, peptidomimetic, or other small molecule. In a preferred embodiment, the disorder characterized by aberrant or unwanted TWIK-7 protein or nucleic acid expression is a CNS disorder, such as a cognitive or neurodegenerative disorder. In another preferred embodiment, the disorder characterized by aberrant or unwanted TWIK-7 protein or nucleic acid expression is a cardiovascular disorder. In another preferred embodiment, the disorder characterized by aberrant or unwanted TWIK-7 protein or nucleic acid expression is a muscular disorder. In another embodiment, the disorder characterized by aberrant or unwanted TWIK-7 activity is a cell proliferation, growth, differentiation, or migration disorder. In another embodiment, the disorder characterized by aberrant or unwanted TWIK-7 activity is a pain disorder, or a disorder characterized by misregulated pain signaling mechanisms.

The present invention also provides diagnostic assays for identifying the presence or absence of a genetic alteration characterized by at least one of (i) aberrant modification or mutation of a gene encoding a TWIK-7 protein; (ii) mis-regulation of the gene; and (iii) aberrant post-translational modification of a TWIK-7 protein, wherein a wild-type form of the gene encodes a protein with a TWIK-7 activity.

In another aspect the invention provides methods for identifying a compound that binds to or modulates the activity of a TWIK-7 protein, by providing an indicator composition comprising a TWIK-7 protein having TWIK-7 activity, contacting the indicator composition with a test compound, and determining the effect of the test compound on TWIK-7 activity in the indicator composition to identify a compound that modulates the activity of a TWIK-7 protein.

Other features and advantages of the invention will be apparent from the following detailed description and claims.

### DETAILED DESCRIPTION OF THE INVENTION

The present invention is based, at least in part, on the discovery of novel molecules, referred to herein as TWIK-7 nucleic acid and protein molecules, which are novel members of the TWIK (for Tandem of P domains in a Weak Inward rectifying K⁺ channel) family of potassium channels. These novel molecules are capable of, for example, modulating a potassium channel mediated activity in a cell, e.g., a neuronal cell, a muscle cell, or a spleen cell.

As used herein, a “potassium channel” includes a protein or polypeptide which is involved in receiving, conducting, and transmitting signals in an electrically excitable cell, e.g., a neuronal cell, a muscle cell (e.g., cardiac muscle), or a spleen cell. Potassium channels are potassium ion selective, and can determine membrane excitability (the ability of, for example, a neuron to respond to a stimulus and convert it into an impulse). Potassium channels can also influence the resting potential of membranes, wave forms and frequencies of action potentials, and thresholds of excitation. Potassium channels are typically expressed in electrically excitable cells, e.g., neurons, muscle, endocrine, and egg cells, and may form heteromultimeric structures, e.g., composed of pore-forming α and cytoplasmic β subunits. Potassium channels may also be found in nonexcitable cells (e.g., spleen cells or prostate cells), where they may play a role in, e.g., signal transduction. Examples of potassium channels include: (1) the voltage-gated potassium channels, (2) the ligand-gated potassium channels, e.g., neurotransmitter-gated potassium channels, and (3) cyclic-nucleotide-gated potassium channels. Voltage-gated and ligand-gated potassium channels are expressed in the brain, e.g., in brainstem monoaminergic and forebrain cholinergic neurons, where they are involved in the release of neurotransmitters, or in the dendrites of hippocampal and neocortical pyramidal cells, where they are involved in the processes of learning and memory formation. For a detailed description of potassium channels, see Kandel E. R.. et al., Principles of Neural Science, second edition, (Elsevier Science Publishing Co., Inc., N.Y. (1985)), the contents of which are incorporated herein by reference. As the TWIK proteins of the present invention may modulate potassium channel mediated activities, they may be useful for developing novel diagnostic and therapeutic agents for potassium channel associated disorders.

As used herein, a “potassium channel associated disorder” includes a disorder, disease or condition which is characterized by a misregulation of a potassium channel mediated activity. Potassium channel associated disorders can detrimentally affect conveyance of sensory impulses from the periphery to the brain and/or conductance of motor impulses from the brain to the periphery; integration of reflexes; interpretation of sensory impulses; and emotional, intellectual (e.g., learning and memory), or motor processes. Examples of potassium channel associated disorders include CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, movement disorders, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further examples of potassium channel associated disorders include cardiac-related disorders. Cardiovascular system disorders in which the TWIK-7 molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia. TWIK-7-mediated or related disorders also include disorders of the musculoskeletal system such as paralysis and muscle weakness, e.g., ataxia, myotonia, and myokymia.

Further disorders in which the TWIK-7 molecules of the invention may be involved are pain disorders. Pain disorders include those that affect pain signaling mechanisms. As used herein, the term “pain signaling mechanisms” includes the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter sensory neurons. These sensory neurons transmit the information to the central nervous system, evoking a perception of pain or discomfort and initiating appropriate protective reflexes. The TWIK-7 molecules of the present invention may be present on these sensory neurons and, thus, may be involved in detecting these noxious chemical, mechanical, or thermal stimuli and transducing this information into membrane depolarization events. Thus, the TWIK-7 molecules by participating in pain signaling mechanisms, may modulate pain elicitation and act as targets for developing novel diagnostic targets and therapeutic agents to control pain. Examples of pain disorders include headache (e.g., tension headache or migraine), back pain, cancer pain, arthritis pain, or neurogenic pain.

Potassium channel disorders also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The TWIK-7 molecules of the present invention are involved in signal transduction mechanisms, which are known to be involved in cellular growth, differentiation, and migration processes. Thus, the TWIK-7 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementia; hepatic disorders; cardiovascular disorders; and hematopoietic and/or myeloproliferative disorders.

TWIK-7-associated or related disorders also include disorders of tissues in which TWIK-7 protein is expressed, e.g., cerebellum, spinal cord, dorsal root ganglia, prostate, uterus and spleen cells. Such disorders include, for example, CNS disorders, muscular disorders, or pain disorders.

As used herein, a “potassium channel mediated activity” includes an activity which involves a potassium channel, e.g., a potassium channel in a neuronal cell, a muscle cell (e.g., cardiac muscle), or a spleen cell, associated with receiving, conducting, and transmitting signals in, for example, the nervous system. Potassium channel mediated activities include release of neurotransmitters, e.g, dopamine or norepinephrine, from cells, e.g., neuronal cells; modulation of resting potential of membranes, wave forms and frequencies of action potentials, and thresholds of excitation; and modulation of processes such as integration of sub-threshold synaptic responses, the conductance of back-propagating action potentials in, for example, neuronal cells or muscle cells, participation in signal transduction pathways, and participation in nociception.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., monkey proteins. Members of a family may also have common functional characteristics.

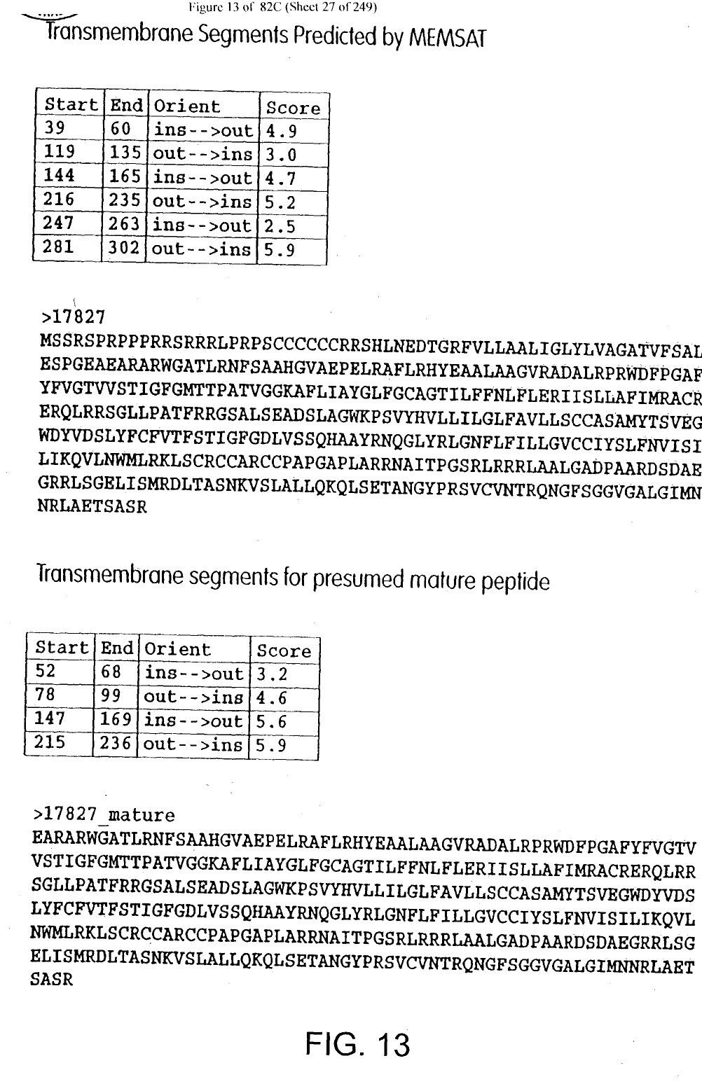

For example, the family of TWIK-7 proteins comprises at least one “transmembrane domain” and preferably four transmembrane domains. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al. (1996) Annual Rev. Neurosci. 19: 235-263, the contents of which are incorporated herein by reference. Amino acid residues 39-60, 144-165, 216-235, and 281-302 of the native TWIK-7 protein are predicted to comprise transmembrane domains (see FIG. 13). Accordingly, TWIK-7 proteins having at least 50-60% homology, preferably about 60-70%, more preferably about 70-80%, or about 80-90% homology with a transmembrane domain of human TWIK-7 are within the scope of the invention.

In another embodiment, a TWIK-7 molecule of the present invention is identified based on the presence of a Pore loop (P-loop). As used herein, the term “Pore loop” or “P-loop” includes an amino acid sequence of about 15-45 amino acid residues in length, preferably about 15-35 amino acid residues in length, and most preferably about 15-25 amino acid residues in length, which is involved in lining the potassium channel pore. A P-loop is typically found between transmembrane domains of potassium channels and is believed to be a major determinant of ion selectivity in potassium channels. Preferably, P-loops contain a G-[HYDROPHOBIC AMINO ACID]-G sequence, e.g., a GYG, GLG, or GFG sequence. P-loops are described in, for example, Warmke et al. (1991) Science 252:1560-1562; Zagotta W. N. et al., (1996) Annu. Rev. Neurosci. 19:235-63 (Pongs, O. (1993) J. Membr. Biol., 136, 1-8; Heginbotham et al. (1994) Biophys. J. 66,1061-1067; Mackinnon, R. (1995) Neuron, and 14, 889-892; Pascual et al., (1995) Neuron., 14, 1055-1063), the contents of which are incorporated herein by reference. Amino acid residues 119-135 and 247-263 of the human TWIK-7 protein comprise P-loops.

In another embodiment, a TWIK-7 molecule of the present invention is identified based on the presence of a “potassium channel protein domain” in the protein or corresponding nucleic acid molecule. As used herein, the term “potassium channel protein domain” includes a protein domain having an amino acid sequence of about 50-350 amino acid residues and having a bit score for the alignment of the sequence to the potassium channel protein domain of about 100-350. Preferably, a potassium channel protein domain includes at least about 200-300, or more preferably about 280 amino acid residues, and has a bit score for the alignment of the sequence to the potassium channel protein domain of at least 20, 30, 40, 50, 60, 70, 80, 90, 100, 110, 120, 130, 140, 150, 160, 170, 180, 190, 200, 210, 220, 230, 240, 250 or higher. The potassium channel protein domain has been assigned ProDom entry 129403. To identify the presence of a potassium channel protein domain in a TWIK-7 protein, and make the determination that a protein of interest has a particular profile, the amino acid sequence of the protein is searched against a database of known protein domains (e.g., the ProDom database) using the default parameters (available online through the Institute National de la Recherche Agronomique, France). A search was performed against the ProDom database resulting in the identification of a potassium channel protein domain in the amino acid sequence of human TWIK-7 (SEQ ID NO:15) at about residues 34-313 of SEQ ID NO:15. The results of the search are set forth in FIG. 14.

In a preferred embodiment, the TWIK-7 molecules of the invention include at least one transmembrane domain, at least one P-loop, and at least one potassium channel protein domain.

Isolated proteins of the present invention, preferably TWIK-7 proteins, have an amino acid sequence sufficiently identical to the amino acid sequence of SEQ ID NO:15 or are encoded by a nucleotide sequence sufficiently identical to SEQ ID NO:14 or 16. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, an “TWIK-7 activity”, “biological activity of TWIK-7” or “functional activity of TWIK-7”, refers to an activity exerted by a TWIK-7 protein, polypeptide or nucleic acid molecule on a TWIK-7 responsive cell or tissue, or on a TWIK-7 protein substrate, as determined in vivo, or in vitro, according to standard techniques. In one embodiment, a TWIK-7 activity is a direct activity, such as an association with a TWIK-7-target molecule. As used herein, a “target molecule” or “binding partner” is a molecule with which a TWIK-7 protein binds or interacts in nature, such that TWIK-7-mediated function is achieved. A TWIK-7 target molecule can be a non-TWIK-7 molecule or a TWIK-7 protein or polypeptide of the present invention. In an exemplary embodiment, a TWIK-7 target molecule is a TWIK-7 ligand, e.g., a potassium channel pore-forming subunit or a potassium channel ligand. Alternatively, a TWIK-7 activity is an indirect activity, such as a cellular signaling activity mediated by interaction of the TWIK-7 protein with a TWIK-7 ligand. The biological activities of TWIK-7 are described herein. For example, the TWIK-7 proteins of the present invention can have one or more of the following activities: (1) interacting with a non-TWIK protein molecule; (2) activating a TWIK-dependent signal transduction pathway; (3) modulating the release of neurotransmitters; (4) modulating membrane excitability; (5) influencing the resting potential of membranes, wave forms and frequencies of action potentials, and thresholds of excitation, (6) modulating processes which underlie learning and memory, such as integration of sub-threshold synaptic responses and the conductance of back-propagating action potentials, and (7) mediating nociception.

Accordingly, another embodiment of the invention features isolated TWIK-7 proteins and polypeptides having a TWIK-7 activity. Preferred proteins are TWIK-7 proteins having at least one transmembrane domain, and, preferably, a TWIK-7 activity. Other preferred proteins are TWIK-7 proteins having a P-loop domain and, preferably, a TWIK-7 activity. Other preferred proteins are TWIK-7 proteins having a potassium channel protein domain and, preferably, a TWIK-7 activity. Yet other preferred proteins are TWIK-7 proteins having at least one transmembrane domain, a P-loop domain, and a potassium channel protein domain and, preferably, a TWIK-7 activity.

Additional preferred proteins have at least one transmembrane domain, and one or more of a P-loop domain and/or a potassium channel protein domain, and are, preferably, encoded by a nucleic acid molecule having a nucleotide sequence which hybridizes under stringent hybridization conditions to a nucleic acid molecule comprising the nucleotide sequence of SEQ ID NO:14 or 16.

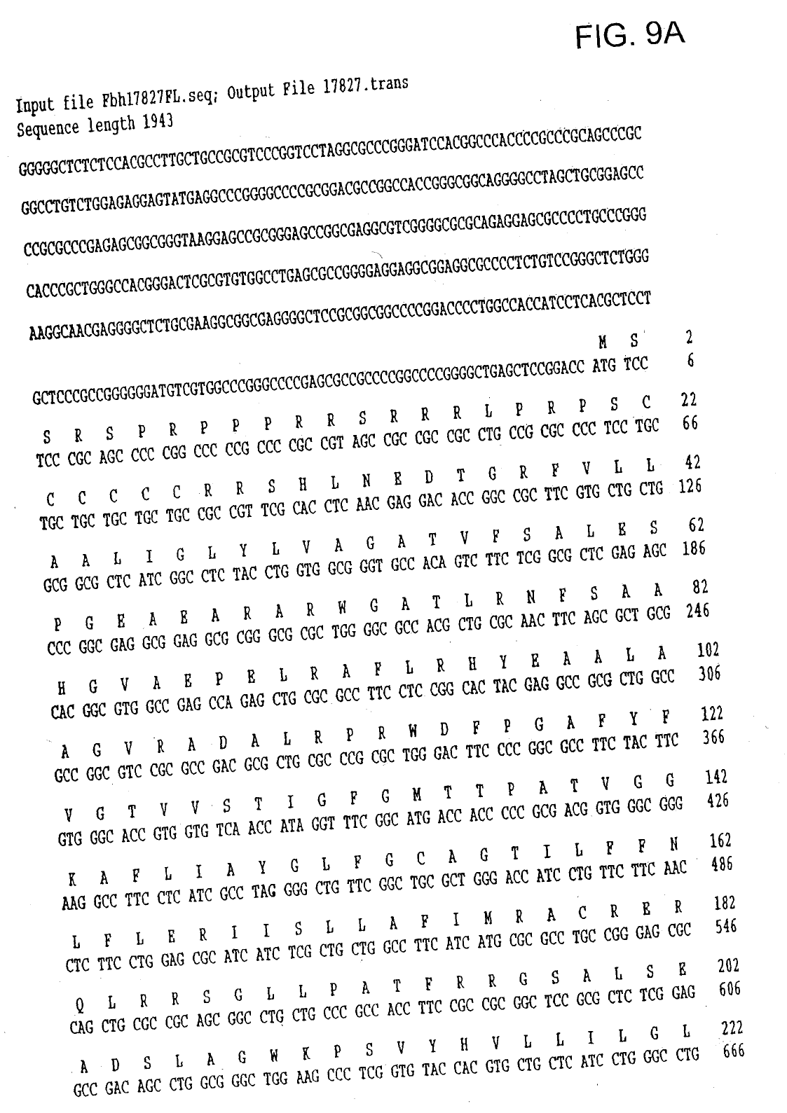

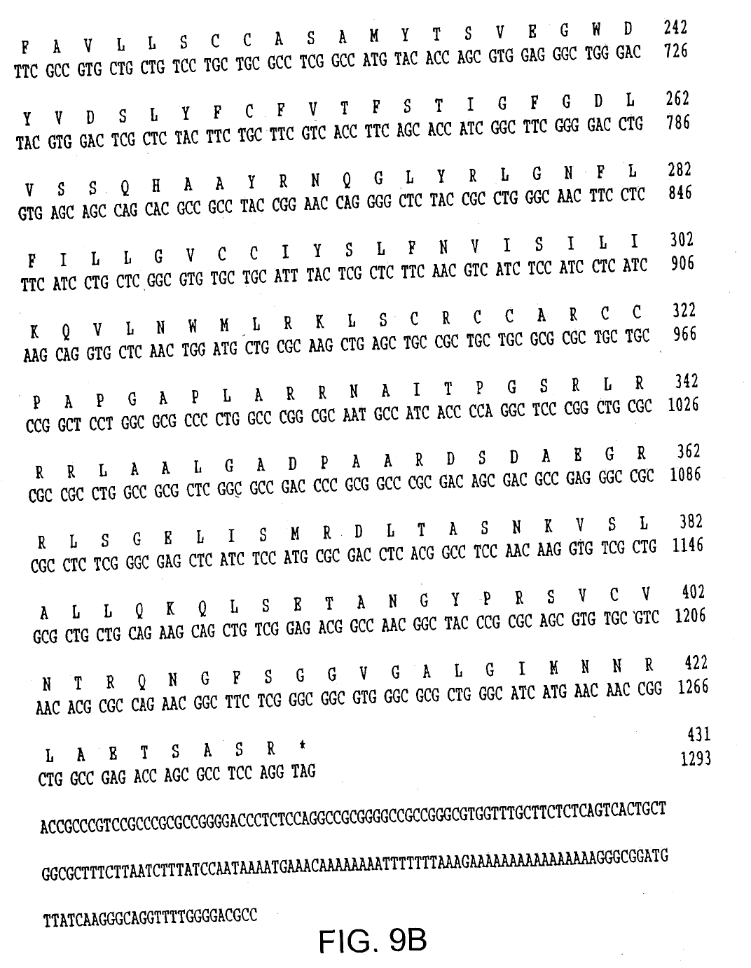

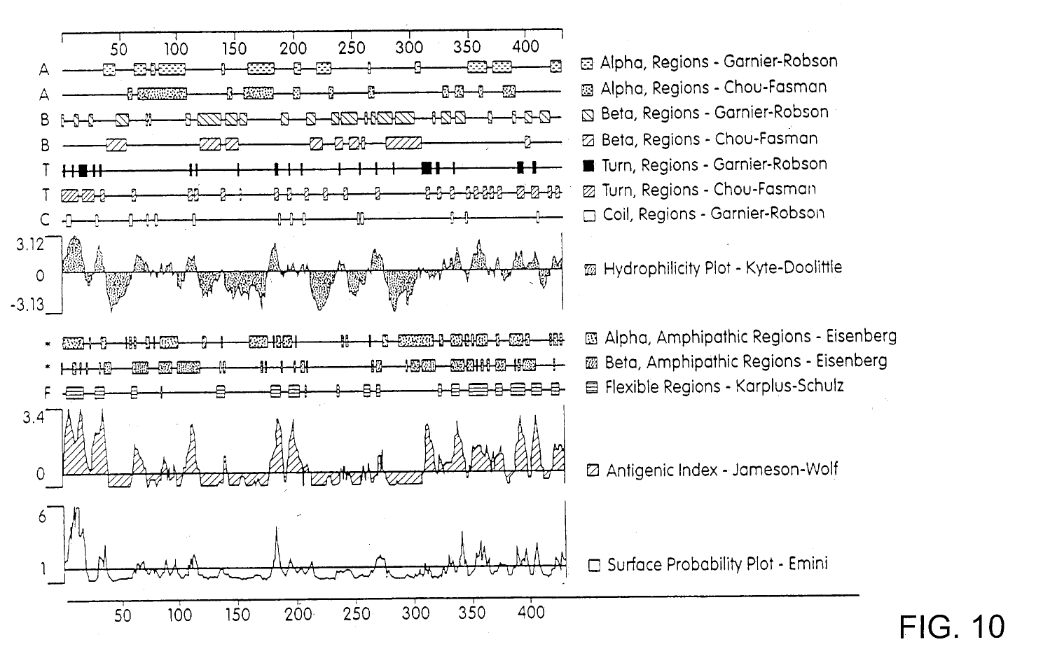

The nucleotide sequence of the isolated human TWIK-7 cDNA and the predicted amino acid sequence of the human TWIK-7 polypeptide are shown in FIGS. 9A-B and in SEQ ID NOs:14 and 15, respectively.

The human TWIK-7 gene, which is approximately 1943 nucleotides in length, encodes a protein having a molecular weight of approximately 48.7 kD and which is approximately 431 amino acid residues in length.

Various aspects of the invention are described in further detail in the following subsections:

I. Isolated Nucleic Acid Molecules

One aspect of the invention pertains to isolated nucleic acid molecules that encode TWIK-7 proteins or biologically active portions thereof, as well as nucleic acid fragments sufficient for use as hybridization probes to identify TWIK-7-encoding nucleic acid molecules (e.g, TWIK-7 mRNA) and fragments for use as PCR primers for the amplification or mutation of TWIK-7 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TWIK-7 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:14 or 16, or a portion thereof, can be isolated using standard molecular biology techniques and the sequence information provided herein. Using all or portion of the nucleic acid sequence of SEQ ID NO:14 or 16 as a hybridization probe, TWIK-7 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:14 or 16 can be isolated by the polymerase chain reaction (PCR) using synthetic oligonucleotide primers designed based upon the sequence of SEQ ID NO:14 or 16.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to TWIK-7 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:14. The sequence of SEQ ID NO:14 corresponds to the human TWIK-7 cDNA. This cDNA comprises sequences encoding the human TWIK-7 protein (i.e., “the coding region”, from nucleotides 466-1758), as well as 5′ untranslated sequences (nucleotides 1-465) and 3′ untranslated sequences (nucleotides 1759-1943). Alternatively, the nucleic acid molecule can comprise only the coding region of SEQ ID NO:14 (e.g., nucleotides 466-1758, corresponding to SEQ ID NO:16).

In another preferred embodiment, an isolated nucleic acid molecule of the invention comprises a nucleic acid molecule which is a complement of the nucleotide sequence shown in SEQ ID NO:14 or 16, or a portion of any of these nucleotide sequences. A nucleic acid molecule which is complementary to the nucleotide sequence shown in SEQ ID NO:14 or 16 is one which is sufficiently complementary to the nucleotide sequence shown in SEQ ID NO:14 or 16 such that it can hybridize to the nucleotide sequence shown in SEQ ID NO:14 or 16, thereby forming a stable duplex.

In still another preferred embodiment, an isolated nucleic acid molecule of the present invention comprises a nucleotide sequence which is at least about 50%, 55%, 60%, 65%, 70%, 73%, 74%, 75%, 80%, 85%, 90%, 95%, 96%, 97%, 98%, 99% or more identical to the entire length of the nucleotide sequence shown in SEQ ID NO:14 or 16, or a portion of any of these nucleotide sequences.

Moreover, the nucleic acid molecule of the invention can comprise only a portion of the nucleic acid sequence of SEQ ID NO:14 or 16, for example, a fragment which can be used as a probe or primer or a fragment encoding a portion of a TWIK-7 protein, e.g., a biologically active portion of a TWIK-7 protein. The nucleotide sequence determined from the cloning of the TWIK-7 gene allows for the generation of probes and primers designed for use in identifying and/or cloning other TWIK-7 family members, as well as TWIK-7 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95, 100, 150, or 200 or more consecutive nucleotides of a sense sequence of SEQ ID NO:14 or 16, of an anti-sense sequence of SEQ ID NO:14 or 16, or of a naturally occurring allelic variant or mutant of SEQ ID NO:14 or 16. In one embodiment, a nucleic acid molecule of the present invention comprises a nucleotide sequence which is greater than 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1910, 1910-1920, 1920-1940 or more nucleotides in length and hybridizes under stringent hybridization conditions to a nucleic acid molecule of SEQ ID NO:14 or 16.

Probes based on the TWIK-7 nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a TWIK-7 protein, such as by measuring a level of a TWIK-7-encoding nucleic acid in a sample of cells from a subject e.g., detecting TWIK-7 mRNA levels or determining whether a genomic TWIK-7 gene has been mutated or deleted.

A nucleic acid fragment encoding a “biologically active portion of a TWIK-7 protein” can be prepared by isolating a portion of the nucleotide sequence of SEQ ID NO:14 or 16 which encodes a polypeptide having a TWIK-7 biological activity (the biological activities of the TWIK-7 proteins are described herein), expressing the encoded portion of the TWIK-7 protein (e.g., by recombinant expression in vitro) and assessing the activity of the encoded portion of the TWIK-7 protein.

The invention further encompasses nucleic acid molecules that differ from the nucleotide sequence shown in SEQ ID NO:14 or 16 due to degeneracy of the genetic code and thus encode the same TWIK-7 proteins as those encoded by the nucleotide sequence shown in SEQ ID NO:14 or 16. In another embodiment, an isolated nucleic acid molecule of the invention has a nucleotide sequence encoding a protein having an amino acid sequence shown in SEQ ID NO:15.

In addition to the TWIK-7 nucleotide sequences shown in SEQ ID NO:14 or 16, it will be appreciated by those skilled in the art that DNA sequence polymorphisms that lead to changes in the amino acid sequences of the TWIK-7 proteins may exist within a population (e.g., the human population). Such genetic polymorphism in the TWIK-7 genes may exist among individuals within a population due to natural allelic variation. As used herein, the terms “gene” and “recombinant gene” refer to nucleic acid molecules which include an open reading frame encoding a TWIK-7 protein, preferably a mammalian TWIK-7 protein, and can further include non-coding regulatory sequences, and introns.

Allelic variants of human TWIK-7 include both functional and non-functional TWIK-7 proteins. Functional allelic variants are naturally occurring amino acid sequence variants of the human TWIK-7 protein that maintain the ability to bind a TWIK-7 ligand or substrate and/or modulate cell proliferation and/or migration mechanisms. Functional allelic variants will typically contain only conservative substitution of one or more amino acids of SEQ ID NO:15, or substitution, deletion or insertion of non-critical residues in non-critical regions of the protein.

Non-functional allelic variants are naturally occurring amino acid sequence variants of the human TWIK-7 protein that do not have the ability to either bind a TWIK-7 ligand and/or modulate any of the TWIK-7 activities described herein. Non-functional allelic variants will typically contain a non-conservative substitution, a deletion, or insertion or premature truncation of the amino acid sequence of SEQ ID NO:15, or a substitution, insertion or deletion in critical residues or critical regions.

The present invention further provides non-human orthologues of the human TWIK-7 protein. Orthologues of the human TWIK-7 protein are proteins that are isolated from non-human organisms and possess the same TWIK-7 ligand binding and/or modulation of membrane excitability activities of the human TWIK-7 protein. Orthologues of the human TWIK-7 protein can readily be identified as comprising an amino acid sequence that is substantially identical to SEQ ID NO:15.

Moreover, nucleic acid molecules encoding other TWIK-7 family members and, thus, which have a nucleotide sequence which differs from the TWIK-7 sequences of SEQ ID NO:14 or 16 are intended to be within the scope of the invention. For example, another TWIK-7 cDNA can be identified based on the nucleotide sequence of human TWIK-7. Moreover, nucleic acid molecules encoding TWIK-7 proteins from different species, and which, thus, have a nucleotide sequence which differs from the TWIK-7 sequences of SEQ ID NO:14 or 16 are intended to be within the scope of the invention. For example, a mouse TWIK-7 cDNA can be identified based on the nucleotide sequence of a human TWIK-7.

Nucleic acid molecules corresponding to natural allelic variants and homologues of the TWIK-7 cDNAs of the invention can be isolated based on their homology to the TWIK-7 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the TWIK-7 cDNAs of the invention can further be isolated by mapping to the same chromosome or locus as the TWIK-7 gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:14 or 16. In other embodiment, the nucleic acid is at least 1910, 1910-1920, 1920-1940, or more nucleotides in length. As used herein, the termn “hybridizes under stringent conditions” is intended to describe conditions for hybridization and washing under which nucleotide sequences at least 60% identical to each other typically remain hybridized to each other. Preferably, the conditions are such that sequences at least about 70%, more preferably at least about 80%, even more preferably at least about 85% or 90% identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50° C., preferably at 55° C., more preferably at 60° C., and even more preferably at 65° C. Preferably, an isolated nucleic acid molecule of the invention that hybridizes under stringent conditions to the sequence of SEQ ID NO:14 or 16 and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the TWIK-7 sequences that may exist in the population, the skilled artisan will further appreciate that changes can be introduced by mutation into the nucleotide sequences of SEQ ID NO:14 or 16, thereby leading to changes in the amino acid sequence of the encoded TWIK-7 proteins, without altering the functional ability of the TWIK-7 proteins. For example, nucleotide substitutions leading to amino acid substitutions at “non-essential” amino acid residues can be made in the sequence of SEQ ID NO:14 or 16. A “non-essential” amino acid residue is a residue that can be altered from the wild-type sequence of TWIK-7 (e.g., the sequence of SEQ ID NO:15) without altering the biological activity, whereas an “essential” amino acid residue is required for biological activity. For example, amino acid residues that are conserved among the TWIK-7 proteins of the present invention, e.g., those present in a transmembrane domain, are predicted to be particularly unamenable to alteration. Furthermore, additional amino acid residues that are conserved between the TWIK-7 proteins of the present invention and other members of the TWIK family are not likely to be amenable to alteration.

Accordingly, another aspect of the invention pertains to nucleic acid molecules encoding TWIK-7 proteins that contain changes in amino acid residues that are not essential for activity. Such TWIK-7 proteins differ in amino acid sequence from SEQ ID NO:15, yet retain biological activity. In one embodiment, the isolated nucleic acid molecule comprises a nucleotide sequence encoding a protein, wherein the protein comprises an amino acid sequence at least about 50%, 55%, 60%, 65%, 67%, 68%, 70%, 75%, 80%, 85%, 90%, 95%, 96%, 97%, 98%, 99% or more identical to SEQ ID NO:15.

An isolated nucleic acid molecule encoding a TWIK-7 protein identical to the protein of SEQ ID NO:15, can be created by introducing one or more nucleotide substitutions, additions or deletions into the nucleotide sequence of SEQ ID NO:14 or 16, such that one or more amino acid substitutions, additions or deletions are introduced into the encoded protein. Mutations can be introduced into SEQ ID NO:14 or 16 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a TWIK-7 protein is preferably replaced with another amino acid residue from the same side chain family. Alternatively, in another embodiment, mutations can be introduced randomly along all or part of a TWIK-7 coding sequence, such as by saturation mutagenesis, and the resultant mutants can be screened for TWIK-7 biological activity to identify mutants that retain activity. Following mutagenesis of SEQ ID NO:14 or 16, the encoded protein can be expressed recombinantly and the activity of the protein can be determined.

In a preferred embodiment, a mutant TWIK-7 protein can be assayed for the ability to (1) interact with a non-TWIK protein molecule; (2) activate a TWIK-dependent signal transduction pathway; (3) modulate the release of neurotransmitters; (4) modulate membrane excitability; (5) influence the resting potential of membranes, wave forms and frequencies of action potentials, and thresholds of excitation, (6) modulate processes which underlie learning and memory, such as integration of sub-threshold synaptic responses and the conductance of back-propagating action potentials, and (7) mediate nociception.

In addition to the nucleic acid molecules encoding TWIK-7 proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TWIK-7 coding strand, or to only a portion thereof. In one embodiment, an antisense nucleic acid molecule is antisense to a “coding region” of the coding strand of a nucleotide sequence encoding TWIK-7. The term “coding region” refers to the region of the nucleotide sequence comprising codons which are translated into amino acid residues (e.g., the coding region of human TWIK-7 corresponds to SEQ ID NO:16). In another embodiment, the antisense nucleic acid molecule is antisense to a “noncoding region” of the coding strand of a nucleotide sequence encoding TWIK-7. The term “noncoding region” refers to 5′ and 3′ sequences which flank the coding region that are not translated into amino acids (i.e., also referred to as 5′ and 3′ untranslated regions).

Given the coding strand sequences encoding TWIK-7 disclosed herein (e.g., SEQ ID NO:16), antisense nucleic acids of the invention can be designed according to the rules of Watson and Crick base pairing. The antisense nucleic acid molecule can be complementary to the entire coding region of TWIK-7 mRNA, but more preferably is an oligonucleotide which is antisense to only a portion of the coding or noncoding region of TWIK-7 mRNA. For example, the antisense oligonucleotide can be complementary to the region surrounding the translation start site of TWIK-7 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a TWIK-7 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave TWIK-7 mRNA transcripts to thereby inhibit translation of TWIK-7 mRNA. A ribozyme having specificity for a TWIK-7-encoding nucleic acid can be designed based upon the nucleotide sequence of a TWIK-7 cDNA disclosed herein (i.e., SEQ ID NO:14 or 16). For example, a derivative of a Tetrahymena L-19 IVS RNA can be constructed in which the nucleotide sequence of the active site is complementary to the nucleotide sequence to be cleaved in a TWIK-7-encoding mRNA. See, e.g., Cech et al. U.S. Pat. No. 4,987,071; and Cech et al. U.S. Pat. No. 5,116,742. Alternatively, TWIK-7 mRNA can be used to select a catalytic RNA having a specific ribonuclease activity from a pool of RNA molecules. See, e.g., Bartel, D. and Szostak, J. W. (1993) Science 261:1411-1418.

Alternatively, TWIK-7 gene expression can be inhibited by targeting nucleotide sequences complementary to the regulatory region of the TWIK-7 (e.g., the TWIK-7 promoter and/or enhancers; e.g., nucleotides 1-465 of SEQ ID NO:14) to form triple helical structures that prevent transcription of the TWIK-7 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the TWIK-7 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad Sci. 93: 14670-675.

PNAs of TWIK-7 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of TWIK-7 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of TWIK-7 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of TWIK-7 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous TWIK-7 gene within a cell line or microorganism may be modified by inserting a heterologous DNA regulatory element into the genome of a stable cell line or cloned microorganism such that the inserted regulatory element is operatively linked with the endogenous TWIK-7 gene. For example, an endogenous TWIK-7 gene which is normally “transcriptionally silent”, i.e., a TWIK-7 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous TWIK-7 gene may be activated by insertion of a promiscuous regulatory element that works across cell types.

A heterologous regulatory element may be inserted into a stable cell line or cloned microorganism, such that it is operatively linked with an endogenous TWIK-7 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated TWIK-7 Proteins and Anti-TWIK-7 Antibodies

One aspect of the invention pertains to isolated TWIK-7 proteins, and biologically active portions thereof, as well as polypeptide fragments suitable for use as immunogens to raise anti-TWIK-7 antibodies. In one embodiment, native TWIK-7 proteins can be isolated from cells or tissue sources by an appropriate purification scheme using standard protein purification techniques. In another embodiment, TWIK-7 proteins are produced by recombinant DNA techniques. Alternative to recombinant expression, a TWIK-7 protein or polypeptide can be synthesized chemically using standard peptide synthesis techniques.

An “isolated” or “purified” protein or biologically active portion thereof is substantially free of cellular material or other contaminating proteins from the cell or tissue source from which the TWIK-7 protein is derived, or substantially free from chemical precursors or other chemicals when chemically synthesized. The language “substantially free of cellular material” includes preparations of TWIK-7 protein in which the protein is separated from cellular components of the cells from which it is isolated or recombinantly produced. In one embodiment, the language “substantially free of cellular material” includes preparations of TWIK-7 protein having less than about 30% (by dry weight) of non-TWIK-7 protein (also referred to herein as a “contaminating protein”), more preferably less than about 20% of non-TWIK-7 protein, still more preferably less than about 10% of non-TWIK-7 protein, and most preferably less than about 5% non-TWIK-7 protein. When the TWIK-7 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of TWIK-7 protein in which the protein is separated from chemical precursors or other chemicals which are involved in the synthesis of the protein. In one embodiment, the language “substantially free of chemical precursors or other chemicals” includes preparations of TWIK-7 protein having less than about 30% (by dry weight) of chemical precursors or non-TWIK-7 chemicals, more preferably less than about 20% chemical precursors or non-TWIK-7 chemicals, still more preferably less than about 10% chemical precursors or non-TWIK-7 chemicals, and most preferably less than about 5% chemical precursors or non-TWIK-7 chemicals.

As used herein, a “biologically active portion” of a TWIK-7 protein includes a fragment of a TWIK-7 protein which participates in an interaction between a TWIK-7 molecule and a non-TWIK-7 molecule. Biologically active portions of a TWIK-7 protein include peptides comprising amino acid sequences sufficiently identical to or derived from the amino acid sequence of the TWIK-7 protein, e.g., the amino acid sequence shown in SEQ ID NO:15, which include less amino acids than the full length TWIK-7 proteins, and exhibit at least one activity of a TWIK-7 protein. Typically, biologically active portions comprise a domain or motif with at least one activity of the TWIK-7 protein, e.g., modulating membrane excitability. A biologically active portion of a TWIK-7 protein can be a polypeptide which is, for example, 25, 30, 35, 40, 45, 50, 75, 100, 125, 150, 175, 200 or more amino acids in length. Biologically active portions of a TWIK-7 protein can be used as targets for developing agents which modulate a TWIK-7 mediated activity, e.g., modulation of membrane excitability.

In one embodiment, a biologically active portion of a TWIK-7 protein comprises at least one transmembrane domain. It is to be understood that a preferred biologically active portion of a TWIK-7 protein of the present invention may contain at least one transmembrane domain and a potassium channel protein domain. Moreover, other biologically active portions, in which other regions of the protein are deleted, can be prepared by recombinant techniques and evaluated for one or more of the functional activities of a native TWIK-7 protein.

In a preferred embodiment, the TWIK-7 protein has an amino acid sequence shown in SEQ ID NO:15. In other embodiments, the TWIK-7 protein is substantially identical to SEQ ID NO:15, and retains the functional activity of the protein of SEQ ID NO:15, yet differs in amino acid sequence due to natural allelic variation or mutagenesis, as described in detail in subsection I above. Accordingly, in another embodiment, the TWIK-7 protein is a protein which comprises an amino acid sequence at least about 50%, 55%, 60%, 65%, 67%, 68%, 70%, 75%, 80%, 85%, 90%, 95%, 96%, 97%, 98%, 99% or more identical to SEQ ID NO:15.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the TWIK-7 amino acid sequence of SEQ ID NO:15 having 220 amino acid residues, at least 50, preferably at least 100, more preferably at least 150, even more preferably at least 175, and even more preferably at least 200 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol (48):444-453 (1970)) algorithm which has been incorporated into the GAP program in the GCG software package (available online through the Genetics Computer Group), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online through the Genetics Computer Group), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to TWIK-7 nucleic acid molecules of the invention. BLAST protein searches can be performed with the XBLAST program, score=100, wordlength=3 to obtain amino acid sequences homologous to TWIK-7 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See the website for the National Center for Biotechnology Information.

The invention also provides TWIK-7 chimeric or fusion proteins. As used herein, a TWIK-7 “chimeric protein” or “fusion protein” comprises a TWIK-7 polypeptide operatively linked to a non-TWIK-7 polypeptide. An “TWIK-7 polypeptide” refers to a polypeptide having an amino acid sequence corresponding to TWIK-7, whereas a “non-TWIK-7 polypeptide” refers to a polypeptide having an amino acid sequence corresponding to a protein which is not substantially homologous to the TWIK-7 protein, e.g., a protein which is different from the TWIK-7 protein and which is derived from the same or a different organism. Within a TWIK-7 fusion protein the TWIK-7 polypeptide can correspond to all or a portion of a TWIK-7 protein. In a preferred embodiment, a TWIK-7 fusion protein comprises at least one biologically active portion of a TWIK-7 protein. In another preferred embodiment, a TWIK-7 fusion protein comprises at least two biologically active portions of a TWIK-7 protein. Within the fusion protein, the term “operatively linked” is intended to indicate that the TWIK-7 polypeptide and the non-TWIK-7 polypeptide are fused in-frame to each other. The non-TWIK-7 polypeptide can be fused to the N-terminus or C-terminus of the TWIK-7 polypeptide.

For example, in one embodiment, the fusion protein is a GST-TWIK-7 fusion protein in which the TWIK-7 sequences are fused to the C-terminus of the GST sequences. Such fusion proteins can facilitate the purification of recombinant TWIK-7.

In another embodiment, the fusion protein is a TWIK-7 protein containing a heterologous signal sequence at its N-terminus. In certain host cells (e.g., mammalian host cells), expression and/or secretion of TWIK-7 can be increased through use of a heterologous signal sequence.

The TWIK-7 fusion proteins of the invention can be incorporated into pharmaceutical compositions and administered to a subject in vivo. The TWIK-7 fusion proteins can be used to affect the bioavailability of a TWIK-7 substrate. Use of TWIK-7 fusion proteins may be useful therapeutically for the treatment of disorders caused by, for example, (i) aberrant modification or mutation of a gene encoding a TWIK-7 protein; (ii) mis-regulation of the TWIK-7 gene; and (iii) aberrant post-translational modification of a TWIK-7 protein.

Moreover, the TWIK-7-fusion proteins of the invention can be used as immunogens to produce anti-TWIK-7 antibodies in a subject, to purify TWIK-7 ligands and in screening assays to identify molecules which inhibit the interaction of TWIK-7 with a TWIK-7 substrate.

Preferably, a TWIK-7 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A TWIK-7-encoding nucleic acid can be cloned into such an expression vector such that the fusion moiety is linked in-frame to the TWIK-7 protein.

The present invention also pertains to variants of the TWIK-7 proteins which function as either TWIK-7 agonists (mimetics) or as TWIK-7 antagonists. Variants of the TWIK-7 proteins can be generated by mutagenesis, e.g., discrete point mutation or truncation of a TWIK-7 protein. An agonist of the TWIK-7 proteins can retain substantially the same, or a subset, of the biological activities of the naturally occurring form of a TWIK-7 protein. An antagonist of a TWIK-7 protein can inhibit one or more of the activities of the naturally occurring form of the TWIK-7 protein by, for example, competitively modulating a TWIK-7-mediated activity of a TWIK-7 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the TWIK-7 protein.

In one embodiment, variants of a TWIK-7 protein which function as either TWIK-7 agonists (mimetics) or as TWIK-7 antagonists can be identified by screening combinatorial libraries of mutants, e.g., truncation mutants, of a TWIK-7 protein for TWIK-7 protein agonist or antagonist activity. In one embodiment, a variegated library of TWIK-7 variants is generated by combinatorial mutagenesis at the nucleic acid level and is encoded by a variegated gene library. A variegated library of TWIK-7 variants can be produced by, for example, enzymatically ligating a mixture of synthetic oligonucleotides into gene sequences such that a degenerate set of potential TWIK-7 sequences is expressible as individual polypeptides, or alternatively, as a set of larger fusion proteins (e.g., for phage display) containing the set of TWIK-7 sequences therein. There are a variety of methods which can be used to produce libraries of potential TWIK-7 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TWIK-7 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a TWIK-7 protein coding sequence can be used to generate a variegated population of TWIK-7 fragments for screening and subsequent selection of variants of a TWIK-7 protein. In one embodiment, a library of coding sequence fragments can be generated by treating a double stranded PCR fragment of a TWIK-7 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the TWIK-7 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TWIK-7 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify TWIK-7 variants (Arkin and Youvan (1992) Proc. Natl. Acad. Sci. USA 89:7811-7815; Delagrave et al. (1993) Protein Engineering 6(3): 327-331).

In one embodiment, cell based assays can be exploited to analyze a variegated TWIK-7 library. For example, a library of expression vectors can be transfected into a cell line, e.g, a neuronal cell line, which ordinarily responds to a TWIK-7 ligand in a particular TWIK-7 ligand-dependent manner. The transfected cells are then contacted with a TWIK-7 ligand and the effect of expression of the mutant on, e.g., membrane excitability of TWIK-7 can be detected. Plasmid DNA can then be recovered from the cells which score for inhibition, or alternatively, potentiation of signaling by the TWIK-7 ligand, and the individual clones further characterized.

An isolated TWIK-7 protein, or a portion or fragment thereof, can be used as an immunogen to generate antibodies that bind TWIK-7 using standard techniques for polyclonal and monoclonal antibody preparation. A full-length TWIK-7 protein can be used or, alternatively, the invention provides antigenic peptide fragments of TWIK-7 for use as immunogens. The antigenic peptide of TWIK-7 comprises at least 8 amino acid residues of the amino acid sequence shown in SEQ ID NO:15 and encompasses an epitope of TWIK-7 such that an antibody raised against the peptide forms a specific immune complex with TWIK-7. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of TWIK-7 that are located on the surface of the protein, e.g. hydrophilic regions, as well as regions with high antigenicity (see, for example, FIG. 10).

A TWIK-7 immunogen typically is used to prepare antibodies by immunizing a suitable subject, (e.g., rabbit, goat, mouse or other mammal) with the immunogen. An appropriate immunogenic preparation can contain, for example, recombinantly expressed TWIK-7 protein or a chemically synthesized TWIK-7 polypeptide. The preparation can further include an adjuvant, such as Freund's complete or incomplete adjuvant, or similar immunostimulatory agent. Immunization of a suitable subject with an immunogenic TWIK-7 preparation induces a polyclonal anti-TWIK-7 antibody response.

Accordingly, another aspect of the invention pertains to anti-TWIK-7 antibodies. The term “antibody” as used herein refers to immunoglobulin molecules and immunologically active portions of immunoglobulin molecules, i.e., molecules that contain an antigen binding site which specifically binds (immunoreacts with) an antigen, such as TWIK-7. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind TWIK-7. The term “monoclonal antibody” or “monoclonal antibody composition”, as used herein, refers to a population of antibody molecules that contain only one species of an antigen binding site capable of immunoreacting with a particular epitope of TWIK-7. A monoclonal antibody composition thus typically displays a single binding affinity for a particular TWIK-7 protein with which it immunoreacts.

Polyclonal anti-TWIK-7 antibodies can be prepared as described above by immunizing a suitable subject with a TWIK-7 immunogen. The anti-TWIK-7 antibody titer in the immunized subject can be monitored over time by standard techniques, such as with an enzyme linked immunosorbent assay (ELISA) using immobilized TWIK-7. If desired, the antibody molecules directed against TWIK-7 can be isolated from the mammal (e.g., from the blood) and further purified by well known techniques, such as protein A chromatography to obtain the IgG fraction. At an appropriate time after immunization, e.g., when the anti-TWIK-7 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a TWIK-7 immunogen as described above, and the culture supernatants of the resulting hybridoma cells are screened to identify a hybridoma producing a monoclonal antibody that binds TWIK-7.

Any of the many well known protocols used for fusing lymphocytes and immortalized cell lines can be applied for the purpose of generating an anti-TWIK-7 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC™ (the American Type Culture Collection, Manassas, Va.). Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind TWIK-7, e.g., using a standard ELISA assay.

Alternative to preparing monoclonal antibody-secreting hybridomas, a monoclonal anti-TWIK-7 antibody can be identified and isolated by screening a recombinant combinatorial immunoglobulin library (e.g., an antibody phage display library) with TWIK-7 to thereby isolate immunoglobulin library members that bind TWIK-7. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et a. PCT International Publication No. WO 92/09690; Ladner et al. PCT International Publication No. WO 90/02809; Fuchs et a. (1991) Bio/Technology 9:1369-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol 226:889-896; Clackson et al. (1991) Nature 352:624-628; Gram et al. (1992) Proc. Natl. Acad. Sci USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-TWIK-7 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Pat. Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567, Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-TWIK-7 antibody (e.g., monoclonal antibody) can be used to isolate TWIK-7 by standard techniques, such as affinity chromatography or immunoprecipitation. An anti-TWIK-7 antibody can facilitate the purification of natural TWIK-7 from cells and of recombinantly produced TWIK-7 expressed in host cells. Moreover, an anti-TWIK-7 antibody can be used to detect TWIK-7 protein (e.g., in a cellular lysate or cell supernatant) in order to evaluate the abundance and pattern of expression of the TWIK-7 protein. Anti-TWIK-7 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a TWIK-7 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., TWIK-7 proteins, mutant forms of TWIK-7 proteins, fusion proteins, and the like).

The recombinant expression vectors of the invention can be designed for expression of TWIK-7 proteins in prokaryotic or eukaryotic cells. For example, TWIK-7 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in TWIK-7 activity assays, (e.g., direct assays or competitive assays described in detail below), or to generate antibodies specific for TWIK-7 proteins, for example. In a preferred embodiment, a TWIK-7 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology. Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the TWIK-7 expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp, San Diego, Calif.).

Alternatively, TWIK-7 proteins can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to TWIK-7 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a TWIK-7 nucleic acid molecule of the invention is introduced, e.g., a TWIK-7 nucleic acid molecule within a recombinant expression vector or a TWIK-7 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a TWIK-7 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a TWIK-7 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a TWIK-7 protein. Accordingly, the invention further provides methods for producing a TWIK-7 protein using the host cells of the invention. In one embodiment, the method comprises culturing the host cell of the invention (into which a recombinant expression vector encoding a TWIK-7 protein has been introduced) in a suitable medium such that a TWIK-7 protein is produced. In another embodiment, the method further comprises isolating a TWIK-7 protein from the medium or the host cell.

The host cells of the invention can also be used to produce non-human transgenic animals. For example, in one embodiment, a host cell of the invention is a fertilized oocyte or an embryonic stem cell into which TWIK-7-coding sequences have been introduced. Such host cells can then be used to create non-human transgenic animals in which exogenous TWIK-7 sequences have been introduced into their genome or homologous recombinant animals in which endogenous TWIK-7 sequences have been altered. Such animals are useful for studying the function and/or activity of a TWIK-7 and for identifying and/or evaluating modulators of TWIK-7 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous TWIK-7 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a TWIK-7-encoding nucleic acid into the male pronuclei of a fertilized oocyte, e.g., by microinjection, retroviral infection, and allowing the oocyte to develop in a pseudopregnant female foster animal. The TWIK-7 cDNA sequence of SEQ ID NO:14 can be introduced as a transgene into the genome of a non-human animal. Alternatively, a nonhuman homologue of a human TWIK-7 gene, such as a mouse or rat TWIK-7 gene, can be used as a transgene. Alternatively, a TWIK-7 gene homologue, such as another TWIK-7 family member, can be isolated based on hybridization to the TWIK-7 cDNA sequences of SEQ ID NO:14 or 16 (described further in subsection I above) and used as a transgene. Intronic sequences and polyadenylation signals can also be included in the transgene to increase the efficiency of expression of the transgene. A tissue-specific regulatory sequence(s) can be operably linked to a TWIK-7 transgene to direct expression of a TWIK-7 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a TWIK-7 transgene in its genome and/or expression of TWIK-7 mRNA in tissues or cells of the animals. A transgenic founder animal can then be used to breed additional animals carrying the transgene. Moreover, transgenic animals carrying a transgene encoding a TWIK-7 protein can further be bred to other transgenic animals carrying other transgenes.

To create a homologous recombinant animal, a vector is prepared which contains at least a portion of a TWIK-7 gene into which a deletion, addition or substitution has been introduced to thereby alter, e.g., functionally disrupt, the TWIK-7 gene. The TWIK-7 gene can be a human gene (e.g., the cDNA of SEQ ID NO:16), but more preferably, is a non-human homologue of a human TWIK-7 gene (e.g., a cDNA isolated by stringent hybridization with the nucleotide sequence of SEQ ID NO:14). For example, a mouse TWIK-7 gene can be used to construct a homologous recombination nucleic acid molecule, e.g., a vector, suitable for altering an endogenous TWIK-7 gene in the mouse genome. In a preferred embodiment, the homologous recombination nucleic acid molecule is designed such that, upon homologous recombination, the endogenous TWIK-7 gene is functionally disrupted (i.e., no longer encodes a functional protein; also referred to as a “knock out” vector). Alternatively, the homologous recombination nucleic acid molecule can be designed such that, upon homologous recombination, the endogenous TWIK-7 gene is mutated or otherwise altered but still encodes functional protein (e.g., the upstream regulatory region can be altered to thereby alter the expression of the endogenous TWIK-7 protein). In the homologous recombination nucleic acid molecule, the altered portion of the TWIK-7 gene is flanked at its 5′ and 3′ ends by additional nucleic acid sequence of the TWIK-7 gene to allow for homologous recombination to occur between the exogenous TWIK-7 gene carried by the homologous recombination nucleic acid molecule and an endogenous TWIK-7 gene in a cell, e.g., an embryonic stem cell. The additional flanking TWIK-7 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced TWIK-7 gene has homologously recombined with the endogenous TWIK-7 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g, a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The TWIK-7 nucleic acid molecules, fragments of TWIK-7 proteins, and anti-TWIK-7 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a TWIK-7 protein or an anti-TWIK-7 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a TWIK-7 protein of the invention has one or more of the following activities: (1) interacting with a non-TWIK protein molecule; (2) activating a TWIK-dependent signal transduction pathway; (3) modulating the release of neurotransmitters; (4) modulating membrane excitability; (5) influencing the resting potential of membranes, wave forms and frequencies of action potentials, and thresholds of excitation, (6) modulating processes which underlie learning and memory, such as integration of sub-threshold synaptic responses and the conductance of back-propagating action potentials, and (7) mediating nociception.

The isolated nucleic acid molecules of the invention can be used, for example, to express TWIK-7 protein (e.g., via a recombinant expression vector in a host cell in gene therapy applications), to detect TWIK-7 mRNA (e.g., in a biological sample) or a genetic alteration in a TWIK-7 gene, and to modulate TWIK-7 activity, as described further below. The TWIK-7 proteins can be used to treat disorders characterized by insufficient or excessive production of a TWIK-7 substrate or production of TWIK-7 inhibitors. In addition, the TWIK-7 proteins can be used to screen for naturally occurring TWIK-7 substrates, to screen for drugs or compounds which modulate TWIK-7 activity, as well as to treat disorders characterized by insufficient or excessive production of TWIK-7 protein or production of TWIK-7 protein forms which have decreased, aberrant or unwanted activity compared to TWIK-7 wild type protein (e.g., CNS disorders such as cognitive and neurodegenerative disorders (e.g., Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, and bipolar affective disorder (e.g., severe bipolar affective (mood) disorder (BP-1) and bipolar affective neurological disorders (e.g., migraine and obesity)); cardiac disorders (e.g., arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia), muscular disorders (e.g., paralysis, muscle weakness (e.g., ataxia, myotonia, and myokymia), muscular dystrophy (e.g., Duchenne muscular dystrophy or myotonic dystrophy), spinal muscular atrophy, congenital myopathies, central core disease, rod myopathy, central nuclear myopathy, Lambert-Eaton syndrome, denervation, and infantile spinal muscular atrophy (Werdnig-Hoffman disease), pain disorders (e.g., headache (e.g., tension headache or migraine), back pain, cancer pain, arthritis pain, or neurogenic pain), and disorders of cellular growth, differentiation, or migration (e.g., cancer, e.g., carcinoma, sarcoma, or leukemia; tumor angiogenesis and metastasis; skeletal dysplasia; neuronal deficiencies resulting from impaired neural induction and patterning; neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementia; hepatic disorders; cardiovascular disorders; and hematopoietic and/or myeloproliferative disorders). Moreover, the anti-TWIK-7 antibodies of the invention can be used to detect and isolate TWIK-7 proteins, to regulate the bioavailability of TWIK-7 proteins, and to modulate TWIK-7 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to TWIK-7 proteins, have a stimulatory or inhibitory effect on, for example, TWIK-7 expression or TWIK-7 activity, or have a stimulatory or inhibitory effect on, for example, the expression or activity of TWIK-7 substrate.

In one embodiment, the invention provides assays for screening candidate or test compounds which are substrates of a TWIK-7 protein or polypeptide or biologically active portion thereof. In another embodiment, the invention provides assays for screening candidate or test compounds which bind to or modulate the activity of a TWIK-7 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. '5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a TWIK-7 protein or biologically active portion thereof is contacted with a test compound and the ability of the test compound to modulate TWIK-7 activity is determined. Determining the ability of the test compound to modulate TWIK-7 activity can be accomplished by monitoring, for example, the release of a neurotransmitter from a cell which expresses TWIK-7. The cell, for example, can be of mammalian origin, e.g., a neuronal cell or a spleen cell.

The ability of the test compound to modulate TWIK-7 binding to a substrate or to bind to TWIK-7 can also be determined. Determining the ability of the test compound to modulate TWIK-7 binding to a substrate can be accomplished, for example, by coupling the TWIK-7 substrate with a radioisotope or enzymatic label such that binding of the TWIK-7 substrate to TWIK-7 can be determined by detecting the labeled TWIK-7 substrate in a complex. Alternatively, TWIK-7 could be coupled with a radioisotope or enzymatic label to monitor the ability of a test compound to modulate TWIK-7 binding to a TWIK-7 substrate in a complex. Determining the ability of the test compound to bind TWIK-7 can be accomplished, for example, by coupling the compound with a radioisotope or enzymatic label such that binding of the compound to TWIK-7 can be determined by detecting the labeled TWIK-7 compound in a complex. For example, compounds (e.g., TWIK-7 substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a TWIK-7 substrate) to interact with TWIK-7 without the labeling of any of the interactants. For example, a microphysiometer can be used to detect the interaction of a compound with TWIK-7 without the labeling of either the compound or the TWIK-7. McConnell, H. M. et al. (1992) Science 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between a compound and TWIK-7.

In another embodiment, an assay is a cell-based assay comprising contacting a cell expressing a TWIK-7 target molecule (e.g., a TWIK-7 substrate) with a test compound and determining the ability of the test compound to modulate (e.g., stimulate or inhibit) the activity of the TWIK-7 target molecule. Determining the ability of the test compound to modulate the activity of a TWIK-7 target molecule can be accomplished, for example, by determining the ability of the TWIK-7 protein to bind to or interact with the TWIK-7 target molecule.

Determining the ability of the TWIK-7 protein, or a biologically active fragment thereof, to bind to or interact with a TWIK-7 target molecule can be accomplished by one of the methods described above for determining direct binding. In a preferred embodiment, determining the ability of the TWIK-7 protein to bind to or interact with a TWIK-7 target molecule can be accomplished by determining the activity of the target molecule. For example, the activity of the target molecule can be determined by detecting induction of a cellular response (i.e., changes in intracellular K⁺ levels), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which a TWIK-7 protein or biologically active portion thereof is contacted with a test compound and the ability of the test compound to bind to the TWIK-7 protein or biologically active portion thereof is determined. Preferred biologically active portions of the TWIK-7 proteins to be used in assays of the present invention include fragments which participate in interactions with non-TWIK-7 molecules, e.g., fragments with high surface probability scores (see, for example, FIG. 10). Binding of the test compound to the TWIK-7 protein can be determined either directly or indirectly as described above. In a preferred embodiment, the assay includes contacting the TWIK-7 protein or biologically active portion thereof with a known compound which binds TWIK-7 to form an assay mixture, contacting the assay mixture with a test compound, and determining the ability of the test compound to interact with a TWIK-7 protein, wherein determining the ability of the test compound to interact with a TWIK-7 protein comprises determining the ability of the test compound to preferentially bind to TWIK-7 or biologically active portion thereof as compared to the known compound.

In another embodiment, the assay is a cell-free assay in which a TWIK-7 protein or biologically active portion thereof is contacted with a test compound and the ability of the test compound to modulate (e.g., stimulate or inhibit) the activity of the TWIK-7 protein or biologically active portion thereof is determined. Determining the ability of the test compound to modulate the activity of a TWIK-7 protein can be accomplished, for example, by determining the ability of the TWIK-7 protein to bind to a TWIK-7 target molecule by one of the methods described above for determining direct binding. Determining the ability of the TWIK-7 protein to bind to a TWIK-7 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon of surface plasmon resonance (SPR) can be used as an indication of real-time reactions between biological molecules.

In an alternative embodiment, determining the ability of the test compound to modulate the activity of a TWIK-7 protein can be accomplished by determining the ability of the TWIK-7 protein to further modulate the activity of a downstream effector of a TWIK-7 target molecule. For example, the activity of the effector molecule on an appropriate target can be determined or the binding of the effector to an appropriate target can be determined as previously described.

In yet another embodiment, the cell-free assay involves contacting a TWIK-7 protein or biologically active portion thereof with a known compound which binds the TWIK-7 protein to form an assay mixture, contacting the assay mixture with a test compound, and determining the ability of the test compound to interact with the TWIK-7 protein, wherein determining the ability of the test compound to interact with the TWIK-7 protein comprises determining the ability of the TWIK-7protein to preferentially bind to or modulate the activity of a TWIK-7 target molecule.

In more than one embodiment of the above assay methods of the present invention, it may be desirable to immobilize either TWIK-7 or its target molecule to facilitate separation of complexed from uncomplexed forms of one or both of the proteins, as well as to accommodate automation of the assay. Binding of a test compound to a TWIK-7 protein, or interaction of a TWIK-7 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/TWIK-7 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or TWIK-7 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of TWIK-7 binding or activity determined using standard techniques.

Other techniques for immobilizing proteins on matrices can also be used in the screening assays of the invention. For example, either a TWIK-7 protein or a TWIK-7 target molecule can be immobilized utilizing conjugation of biotin and streptavidin. Biotinylated TWIK-7 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with TWIK-7 protein or target molecules but which do not interfere with binding of the TWIK-7 protein to its target molecule can be derivatized to the wells of the plate, and unbound target or TWIK-7 protein trapped in the wells by antibody conjugation. Methods for detecting such complexes, in addition to those described above for the GST-immobilized complexes, include immunodetection of complexes using antibodies reactive with the TWIK-7 protein or target molecule, as well as enzyme-linked assays which rely on detecting an enzymatic activity associated with the TWIK-7 protein or target molecule.

In another embodiment, modulators of TWIK-7 expression are identified in a method wherein a cell is contacted with a candidate compound and the expression of TWIK-7 mRNA or protein in the cell is determined. The level of expression of TWIK-7 mRNA or protein in the presence of the candidate compound is compared to the level of expression of TWIK-7 mRNA or protein in the absence of the candidate compound. The candidate compound can then be identified as a modulator of TWIK-7 expression based on this comparison. For example, when expression of TWIK-7 mRNA or protein is greater (statistically significantly greater) in the presence of the candidate compound than in its absence, the candidate compound is identified as a stimulator of TWIK-7 mRNA or protein expression. Alternatively, when expression of TWIK-7 mRNA or protein is less (statistically significantly less) in the presence of the candidate compound than in its absence, the candidate compound is identified as an inhibitor of TWIK-7 mRNA or protein expression. The level of TWIK-7 mRNA or protein expression in the cells can be determined by methods described herein for detecting TWIK-7 mRNA or protein.

In yet another aspect of the invention, the TWIK-7 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with TWIK-7 (“TWIK-7-binding proteins” or “TWIK-7-bp”) and are involved in TWIK-7 activity. Such TWIK-7-binding proteins are also likely to be involved in the propagation of signals by the TWIK-7 proteins or TWIK-7 targets as, for example, downstream elements of a TWIK-7-mediated signaling pathway. Alternatively, such TWIK-7-binding proteins are likely to be TWIK-7 inhibitors.

The two-hybrid system is based on the modular nature of most transcription factors, which consist of separable DNA-binding and activation domains. Briefly, the assay utilizes two different DNA constructs. In one construct, the gene that codes for a TWIK-7 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TWIK-7-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the TWIK-7 protein.

In another aspect, the invention pertains to a combination of two or more of the assays described herein. For example, a modulating agent can be identified using a cell-based or a cell free assay, and the ability of the agent to modulate the activity of a TWIK-7 protein can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., a TWIK-7 modulating agent, an antisense TWIK-7 nucleic acid molecule, a TWIK-7-specific antibody, or a TWIK-7-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the TWIK-7 nucleotide sequences, described herein, can be used to map the location of the TWIK-7 genes on a chromosome. The mapping of the TWIK-7 sequences to chromosomes is an important first step in correlating these sequences with genes associated with disease.

Briefly, TWIK-7 genes can be mapped to chromosomes by preparing PCR primers (preferably 15-25 bp in length) from the TWIK-7 nucleotide sequences. Computer analysis of the TWIK-7 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the TWIK-7 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the TWIK-7 nucleotide sequences to design oligonucleotide primers, sublocalization can be achieved with panels of fragments from specific chromosomes. Other mapping strategies which can similarly be used to map a TWIK-7 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the TWIK-7 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The TWIK-7 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the TWIK-7 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The TWIK-7 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:14 can comfortably provide positive individual identification with a panel of perhaps 10 to 1,000 primers which each yield a noncoding amplified sequence of 100 bases. If predicted coding sequences, such as those in SEQ ID NO:16 are used, a more appropriate number of primers for positive individual identification would be 500-2,000.

If a panel of reagents from TWIK-7 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of TWIK-7 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:14 are particularly appropriate for this use as greater numbers of polymorphisms occur in the noncoding regions, making it easier to differentiate individuals using this technique. Examples of polynucleotide reagents include the TWIK-7 nucleotide sequences or portions thereof, e.g., fragments derived from the noncoding regions of SEQ ID NO:14 having a length of at least 20 bases, preferably at least 30 bases.

The TWIK-7 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such TWIK-7 probes can be used to identify tissue by species and/or by organ type.

In a similar fashion, these reagents, e.g., TWIK-7 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining TWIK-7 protein and/or nucleic acid expression as well as TWIK-7 activity, in the context of a biological sample (e.g., blood, serum, cells, tissue) to thereby determine whether an individual is afflicted with a disease or disorder, or is at risk of developing a disorder, associated with aberrant or unwanted TWIK-7 expression or activity. The invention also provides for prognostic (or predictive) assays for determining whether an individual is at risk of developing a disorder associated with TWIK-7 protein, nucleic acid expression or activity. For example, mutations in a TWIK-7 gene can be assayed in a biological sample. Such assays can be used for prognostic or predictive purpose to thereby prophylactically treat an individual prior to the onset of a disorder characterized by or associated with TWIK-7 protein, nucleic acid expression or activity.

Another aspect of the invention pertains to monitoring the influence of agents (e.g., drugs, compounds) on the expression or activity of TWIK-7 in clinical trials.

These and other agents are described in further detail in the following sections.

1. Diagnostic Assays

An exemplary method for detecting the presence or absence of TWIK-7 protein or nucleic acid in a biological sample involves obtaining a biological sample from a test subject and contacting the biological sample with a compound or an agent capable of detecting TWIK-7 protein or nucleic acid (e.g., mRNA, or genomic DNA) that encodes TWIK-7 protein such that the presence of TWIK-7 protein or nucleic acid is detected in the biological sample. A preferred agent for detecting TWIK-7 mRNA or genomic DNA is a labeled nucleic acid probe capable of hybridizing to TWIK-7 mRNA or genomic DNA. The nucleic acid probe can be, for example, the TWIK-7 nucleic acid set forth in SEQ ID NO:14 or 16, or a portion thereof, such as an oligonucleotide of at least 15, 30, 50, 100, 250 or 500 nucleotides in length and sufficient to specifically hybridize under stringent conditions to TWIK-7 mRNA or genomic DNA. Other suitable probes for use in the diagnostic assays of the invention are described herein.

A preferred agent for detecting TWIK-7 protein is an antibody capable of binding to TWIK-7 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect TWIK-7 mRNA, protein, or genomic DNA in a biological sample in vitro as well as in vivo. For example, in vitro techniques for detection of TWIK-7 mRNA include Northern hybridizations and in situ hybridizations. In vitro techniques for detection of TWIK-7 protein include enzyme linked immunosorbent assays (ELISAs), Western blots, immunoprecipitations and immunofluorescence. In vitro techniques for detection of TWIK-7 genomic DNA include Southern hybridizations. Furthermore, in vivo techniques for detection of TWIK-7 protein include introducing into a subject a labeled anti-TWIK-7 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting TWIK-7 protein, mRNA, or genomic DNA, such that the presence of TWIK-7 protein, mRNA or genomic DNA is detected in the biological sample, and comparing the presence of TWIK-7 protein, mRNA or genomic DNA in the control sample with the presence of TWIK-7 protein, mRNA or genomic DNA in the test sample.

The invention also encompasses kits for detecting the presence of TWIK-7 in a biological sample. For example, the kit can comprise a labeled compound or agent capable of detecting TWIK-7 protein or mRNA in a biological sample; means for determining the amount of TWIK-7 in the sample; and means for comparing the amount of TWIK-7 in the sample with a standard. The compound or agent can be packaged in a suitable container. The kit can further comprise instructions for using the kit to detect TWIK-7 protein or nucleic acid.

2. Prognostic Assays

The diagnostic methods described herein can furthermore be utilized to identify subjects having or at risk of developing a disease or disorder associated with aberrant or unwanted TWIK-7 expression or activity. As used herein, the term “aberrant” includes a TWIK-7 expression or activity which deviates from the wild type TWIK-7 expression or activity. Aberrant expression or activity includes increased or decreased expression or activity, as well as expression or activity which does not follow the wild type developmental pattern of expression or the subcellular pattern of expression. For example, aberrant TWIK-7 expression or activity is intended to include the cases in which a mutation in the TWIK-7 gene causes the TWIK-7 gene to be under-expressed or over-expressed and situations in which such mutations result in a non-functional TWIK-7 protein or a protein which does not function in a wild-type fashion, e.g., a protein which does not interact with a TWIK-7 substrate, e.g., a non-potassium channel subunit or ligand, or one which interacts with a non-TWIK-7 substrate, e.g. a non-potassium channel subunit or ligand. As used herein, the term “unwanted” includes an unwanted phenomenon involved in a biological response such as cellular proliferation. For example, the term unwanted includes a TWIK-7 expression or activity which is undesirable in a subject.

The assays described herein, such as the preceding diagnostic assays or the following assays, can be utilized to identify a subject having or at risk of developing a disorder associated with a misregulation in TWIK-7 protein activity or nucleic acid expression, such as a CNS disorder (e.g., a cognitive or neurodegenerative disorder, a pain disorder, a muscular disorder, or a cellular proliferation, growth, differentiation, or migration disorder), a cardiovascular disorder, or a pain disorder. Alternatively, the prognostic assays can be utilized to identify a subject having or at risk for developing a disorder associated with a misregulation in TWIK-7 protein activity or nucleic acid expression, such as a CNS disorder, a cardiovascular disorder, or a pain disorder, or a cellular proliferation, growth, differentiation, or migration disorder. Thus, the present invention provides a method for identifying a disease or disorder associated with aberrant or unwanted TWIK-7 expression or activity in which a test sample is obtained from a subject and TWIK-7 protein or nucleic acid (e.g., mRNA or genomic DNA) is detected, wherein the presence of TWIK-7 protein or nucleic acid is diagnostic for a subject having or at risk of developing a disease or disorder associated with aberrant or unwanted TWIK-7 expression or activity. As used herein, a “test sample” refers to a biological sample obtained from a subject of interest. For example, a test sample can be a biological fluid (e.g., cerebrospinal fluid or serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted TWIK-7 expression or activity. For example, such methods can be used to determine whether a subject can be effectively treated with an agent for a CNS disorder, a muscular disorder, a pain disorder, or a cellular proliferation, growth, differentiation, or migration disorder. Thus, the present invention provides methods for determining whether a subject can be effectively treated with an agent for a disorder associated with aberrant or unwanted TWIK-7 expression or activity in which a test sample is obtained and TWIK-7 protein or nucleic acid expression or activity is detected (e.g., wherein the abundance of TWIK-7 protein or nucleic acid expression or activity is diagnostic for a subject that can be administered the agent to treat a disorder associated with aberrant or unwanted TWIK-7 expression or activity).

The methods of the invention can also be used to detect genetic alterations in a TWIK-7 gene, thereby determining if a subject with the altered gene is at risk for a disorder characterized by misregulation in TWIK-7 protein activity or nucleic acid expression, such as a CNS disorder, a cardiovascular disorder, a pain disorder, or a disorder of cellular growth, differentiation, or migration. In preferred embodiments, the methods include detecting, in a sample of cells from the subject, the presence or absence of a genetic alteration characterized by at least one of an alteration affecting the integrity of a gene encoding a TWIK-7-protein, or the mis-expression of the TWIK-7 gene. For example, such genetic alterations can be detected by ascertaining the existence of at least one of 1) a deletion of one or more nucleotides from a TWIK-7 gene; 2) an addition of one or more nucleotides to a TWIK-7 gene; 3) a substitution of one or more nucleotides of a TWIK-7 gene, 4) a chromosomal rearrangement of a TWIK-7 gene; 5) an alteration in the level of a messenger RNA transcript of a TWIK-7 gene, 6) aberrant modification of a TWIK-7 gene, such as of the methylation pattern of the genomic DNA, 7) the presence of a non-wild type splicing pattern of a messenger RNA transcript of a TWIK-7 gene, 8) a non-wild type level of a TWIK-7-protein, 9) allelic loss of a TWIK-7 gene, and 10) inappropriate post-translational modification of a TWIK-7-protein. As described herein, there are a large number of assays known in the art which can be used for detecting alterations in a TWIK-7 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the TWIK-7-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a TWIK-7 gene under conditions such that hybridization and amplification of the TWIK-7-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a TWIK-7 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in TWIK-7 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in TWIK-7 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the TWIK-7 gene and detect mutations by comparing the sequence of the sample TWIK-7 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the TWIK-7 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type TWIK-7 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in TWIK-7 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a TWIK-7 sequence, e.g., a wild-type TWIK-7 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in TWIK-7 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control TWIK-7 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a TWIK-7 gene.

Furthermore, any cell type or tissue in which TWIK-7 is expressed may be utilized in the prognostic assays described herein.

3. Monitoring of Effects During Clinical Trials

Monitoring the influence of agents (e.g., drugs) on the expression or activity of a TWIK-7 protein (e.g., the modulation of cell proliferation and/or migration) can be applied not only in basic drug screening, but also in clinical trials. For example, the effectiveness of an agent determined by a screening assay as described herein to increase TWIK-7 gene expression, protein levels, or upregulate TWIK-7 activity, can be monitored in clinical trials of subjects exhibiting decreased TWIK-7 gene expression, protein levels, or downregulated TWIK-7 activity. Alternatively, the effectiveness of an agent determined by a screening assay to decrease TWIK-7 gene expression, protein levels, or downregulate TWIK-7 activity, can be monitored in clinical trials of subjects exhibiting increased TWIK-7 gene expression, protein levels, or upregulated TWIK-7 activity. In such clinical trials, the expression or activity of a TWIK-7 gene, and preferably, other genes that have been implicated in, for example, a TWIK-7-associated disorder can be used as a “read out” or markers of the phenotype of a particular cell.

For example, and not by way of limitation, genes, including TWIK-7, that are modulated in cells by treatment with an agent (e.g., compound, drug or small molecule) which modulates TWIK-7 activity (e.g., identified in a screening assay as described herein) can be identified. Thus, to study the effect of agents on TWIK-7-associated disorders (e.g., disorders characterized by deregulated cell proliferation and/or migration), for example, in a clinical trial, cells can be isolated and RNA prepared and analyzed for the levels of expression of TWIK-7 and other genes implicated in the TWIK-7-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of TWIK-7 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a TWIK-7 protein, mRNA, or genomic DNA in the preadministration sample; (iii) obtaining one or more post-administration samples from the subject; (iv) detecting the level of expression or activity of the TWIK-7 protein, mRNA, or genomic DNA in the post-administration samples; (v) comparing the level of expression or activity of the TWIK-7 protein, mRNA, or genomic DNA in the pre-administration sample with the TWIK-7 protein, mRNA, or genomic DNA in the post administration sample or samples; and (vi) altering the administration of the agent to the subject accordingly. For example, increased administration of the agent may be desirable to increase the expression or activity of TWIK-7 to higher levels than detected, i.e., to increase the effectiveness of the agent. Alternatively, decreased administration of the agent may be desirable to decrease expression or activity of TWIK-7 to lower levels than detected, i.e. to decrease the effectiveness of the agent. According to such an embodiment, TWIK-7 expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted TWIK-7 expression or activity, e.g. a CNS disorder, a cardiovascular disorder, a pain disorder, or a cellular proliferation, growth, differentiation, or migration disorder. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the TWIK-7 molecules of the present invention or TWIK-7 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted TWIK-7 expression or activity, by administering to the subject a TWIK-7 or an agent which modulates TWIK-7 expression or at least one TWIK-7 activity. Subjects at risk for a disease which is caused or contributed to by aberrant or unwanted TWIK-7 expression or activity can be identified by, for example, any or a combination of diagnostic or prognostic assays as described herein. Administration of a prophylactic agent can occur prior to the manifestation of symptoms characteristic of the TWIK-7 aberrancy, such that a disease or disorder is prevented or, alternatively, delayed in its progression. Depending on the type of TWIK-7 aberrancy, for example, a TWIK-7, TWIK-7 agonist or TWIK-7 antagonist agent can be used for treating the subject. The appropriate agent can be determined based on screening assays described herein.

2. Therapeutic Methods

Another aspect of the invention pertains to methods of modulating TWIK-7 expression or activity for therapeutic purposes. Accordingly, in an exemplary embodiment, the modulatory method of the invention involves contacting a cell with a TWIK-7 or agent that modulates one or more of the activities of TWIK-7 protein activity associated with the cell. An agent that modulates TWIK-7 protein activity can be an agent as described herein, such as a nucleic acid or a protein, a naturally-occurring target molecule of a TWIK-7 protein (e.g., a TWIK-7 substrate), a TWIK-7 antibody, a TWIK-7 agonist or antagonist, a peptidomimetic of a TWIK-7 agonist or antagonist, or other small molecule. In one embodiment, the agent stimulates one or more TWIK-7 activities. Examples of such stimulatory agents include active TWIK-7 protein and a nucleic acid molecule encoding TWIK-7 that has been introduced into the cell. In another embodiment, the agent inhibits one or more TWIK-7 activities. Examples of such inhibitory agents include antisense TWIK-7 nucleic acid molecules, anti-TWIK-7 antibodies, and TWIK-7 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a TWIK-7 protein or nucleic acid molecule. In one embodiment, the method involves administering an agent (e.g., an agent identified by a screening assay described herein), or combination of agents that modulates (e.g., upregulates or downregulates) TWIK-7 expression or activity. In another embodiment, the method involves administering a TWIK-7 protein or nucleic acid molecule as therapy to compensate for reduced, aberrant, or unwanted TWIK-7 expression or activity.

Stimulation of TWIK-7 activity is desirable in situations in which TWIK-7 is abnormally downregulated and/or in which increased TWIK-7 activity is likely to have a beneficial effect. Likewise, inhibition of TWIK-7 activity is desirable in situations in which TWIK-7 is abnormally upregulated and/or in which decreased TWIK-7 activity is likely to have a beneficial effect.

3. Pharmacogenomics

The TWIK-7 molecules of the present invention, as well as agents, or modulators which have a stimulatory or inhibitory effect on TWIK-7 activity (e.g., TWIK-7 gene expression) as identified by a screening assay described herein can be administered to individuals to treat (prophylactically or therapeutically) TWIK-7-associated disorders (e.g., proliferative disorders) associated with aberrant or unwanted TWIK-7 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a TWIK-7 molecule or TWIK-7 modulator as well as tailoring the dosage and/or therapeutic regimen of treatment with a TWIK-7 molecule or TWIK-7 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a TWIK-7 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a TWIK-7 molecule or TWIK-7 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a TWIK-7 molecule or TWIK-7 modulator, such as a modulator identified by one of the exemplary screening assays described herein.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human TWIK-7 cDNA

In this example, the identification and characterization of the gene encoding human TWIK-7 (clone Fbh17827) is described.

Isolation of the TWIK-7 cDNA

The invention is based, at least in part, on the discovery of a human gene encoding a novel protein, referred to herein as TWIK-7. The entire sequence of the human clone Fbh17827 was determined and found to contain an open reading frame termed human “TWIK-7.”

The nucleotide sequence encoding the human TWIK-7 protein is shown in FIGS. 9A-B and is set forth as SEQ ID NO:14. The protein encoded by this nucleic acid comprises about 431 amino acids and has the amino acid sequence shown in FIGS. 9A-B and set forth as SEQ ID NO:15. The coding region (open reading frame) of SEQ ID NO:14 is set forth as SEQ ID NO:16.

Analysis of the Human TWIK-7 Molecule

A BLASTN 2.0 search against the NRN database, using a score of 100 and a wordlength of 12 (Altschul et al. (1990) J. Mol. Biol. 215:403) of the nucleotide sequence of human TWIK-7 revealed that human TWIK-7 is 55% identical to Streptomyces violaceoruber Tu22 granaticin biosynthetic gene cluster (Accession Number AJ011500) over nucleotides 15-1807, is 55% identical over nucleotides 12-1710, is 55% identical over nucleotides 25-1107, is 54% identical over nucleotides 343-1689, is 54% identical over nucleotides 27-1812, is 54% identical over nucleotides 56-1821, is 53% identical over nucleotides 2-1815, is 53% identical over nucleotides 56-1811, is 53% identical over nucleotides 56-1685, is 56% identical over nucleotides 1809-62, is 54% identical over nucleotides 1185-9, is 54% identical over nucleotides 1709-117, is 53% identical over nucleotides 1759-128, is 55% identical over nucleotides 889-21, and is 53% identical over nucleotides 1783-248, is 55% identical over nucleotides 843-363. This search further revealed that human TWIK-7 is 56% identical to Sequence 2 from Patent WO9516779 (Accession Number A45258) over nucleotides 13-1815, is 55% identical over nucleotides 6-1842, is 55% identical over nucleotides 1-1809, is 55% identical over nucleotides 1816-10, and is 54% identical over nucleotides 1823-521.

A BLASTN 2.0 search against the dbEST database, using a score of 100 and a wordlength of 12 (Altschul et al. (1990) J. Mol. Biol. 215:403) of the nucleotide sequence of human TWIK-7 revealed that human TWIK-7 is 94% identical to UI-M-BH2.2-ao1-b-03-0-UI.s1 NIH_BMAP_M_S3.2 Mus musculus cDNA clone UI-M-BH2.2-ao1-b-03-0-UI 3′, mRNA sequence (Accession Number AW122613) over nucleotides 1766-1429, and is 57% identical over nucleotides 737-395. This search further revealed that human TWIK-7 is 93% identical to UI-R-C2-mq-e-12-0-UI.s1 UI-R-C2 Rattus norvegicus cDNA clone UI-R-C2-mq-e-12-0-UI 3′ over nucleotides 1766-1429, and is 56% identical over nucleotides 682-395. This search further revealed that human TWIK-7 is 99% identical to wb18h05.x1 NCI_CGAP_GC6 Homo sapiens cDNA clone IMAGE:2306073 3′, mRNA sequence (Accession Number AI825537) over nucleotides 1895-1675, and is 64% identical over nucleotides 779-663.

A BLASTN 2.0 search against the PatentDbPreviewNuc database, using a score of 100 and a wordlength of 12 (Altschul et al. (1990) J. Mol. Biol. 215:403) of the nucleotide sequence of human TWIK-7 revealed that human TWIK-7 is 55% identical to METH1 and METH2 polynucleotides (Accession Number AC28375)(WO99/37660) over nucleotides 2-1799, is 54% identical over nucleotides 345-1846, is 54% identical over nucleotides 1828-5, and is 55% identical over nucleotides 1160-13.

A BLASTN 2.0 search against the gsnuc database, using a score of 100 and a wordlength of 12 (Altschul et al. (1990) J. Mol. Biol. 215:403) of the nucleotide sequence of human TWIK-7 revealed that human TWIK-7 is 54% identical to BamHI J-1 fragment carrying sequence characteristic of productive pseudorabies virus (Accession Number Q10212) over nucleotides 438-1815, is 56% identical over nucleotides 10-2540, is 56% identical over nucleotides 1923-15, and is 54% identical over nucleotides 1859-112. This search further revealed that human TWIK-7 is 54% identical to BamHI J-1 fragment carrying sequences characteristic of latent pseudorabies virus (Accession Number Q10543) over nucleotides 438-1815, is 56% identical over nucleotides 10-740, is 56% identical over nucleotides 1923-15, and is 54% identical over nucleotides 1859-112.

A BLASTX 2.0 search against the NRP/protot database, using a score of 100, a wordlength of 3, and a Blosum 62 matrix (Altschul et al. (1990) J. Mol. Biol. 215:403), of the translated nucleotide sequence of human TWIK-7 revealed that human TWIK-7 is 37% identical to Caenorhabditis elegans ‘similar to potassium channel protein’ (Accession Number Z75543) over nucleotides 541-1413. This search further revealed that human TWIK-7 is 28% identical to Boreogadus saida antifreeze glycopeptide AFGP polyprotein precursor (Accession Number U43200) over translated nucleotides 335-1711, is 28% identical over translated nucleotides 413-1750, is 27% identical over translated nucleotides 113-1582, is 26% identical over translated nucleotides 254-1708, is 27% identical over translated nucleotides 233-1600, is 32% identical over translated nucleotides 32-1432, is 27% identical over translated nucleotides 572-1837, is 29% identical over translated nucleotides 1127-1843, is 26% identical over translated nucleotides 11-826, is 27% identical over translated nucleotides 549-100, is 25% identical over translated nucleotides 813-28, is 29% identical over translated nucleotides 579-70, is 29% identical over translated nucleotides 579-28, is 31% identical over translated nucleotides 552-28, is 27% identical over translated nucleotides 579-133, is 30% identical over translated nucleotides 552-28, is 29% identical over translated nucleotides 549-88, is 30% identical over translated nucleotides 555-82, is 22% identical over translated nucleotides 1199-558, is 23% identical over translated nucleotides 1199-588, is 30% identical over translated nucleotides 555-79, is 22% identical over translated nucleotides 1142-558, is 34% identical over translated nucleotides 579-337, is 22% identical over translated nucleotides 1813-992, is 23% identical over translated nucleotides 1219-518, is 21% identical over translated nucleotides 1747-641, is 23% identical over translated nucleotides 1842-1408, is 22% identical over translated nucleotides 1204-518, is 27% identical over translated nucleotides 785-582, and is 27% identical over translated nucleotides 1842-1648.

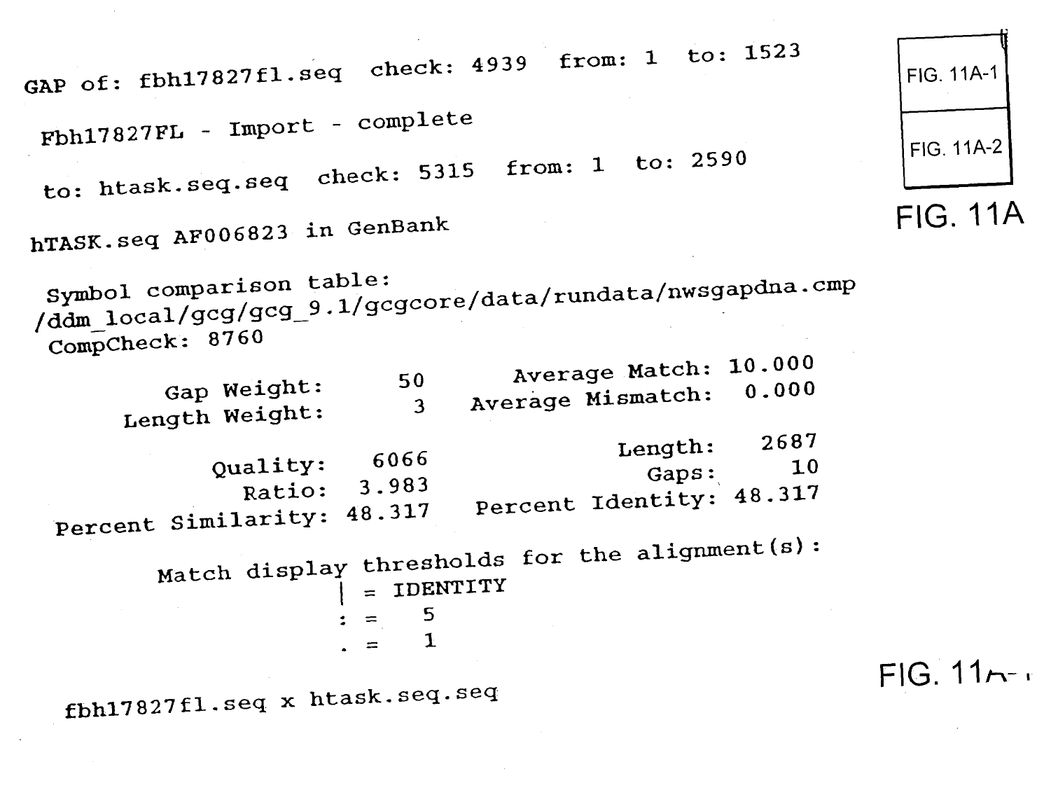

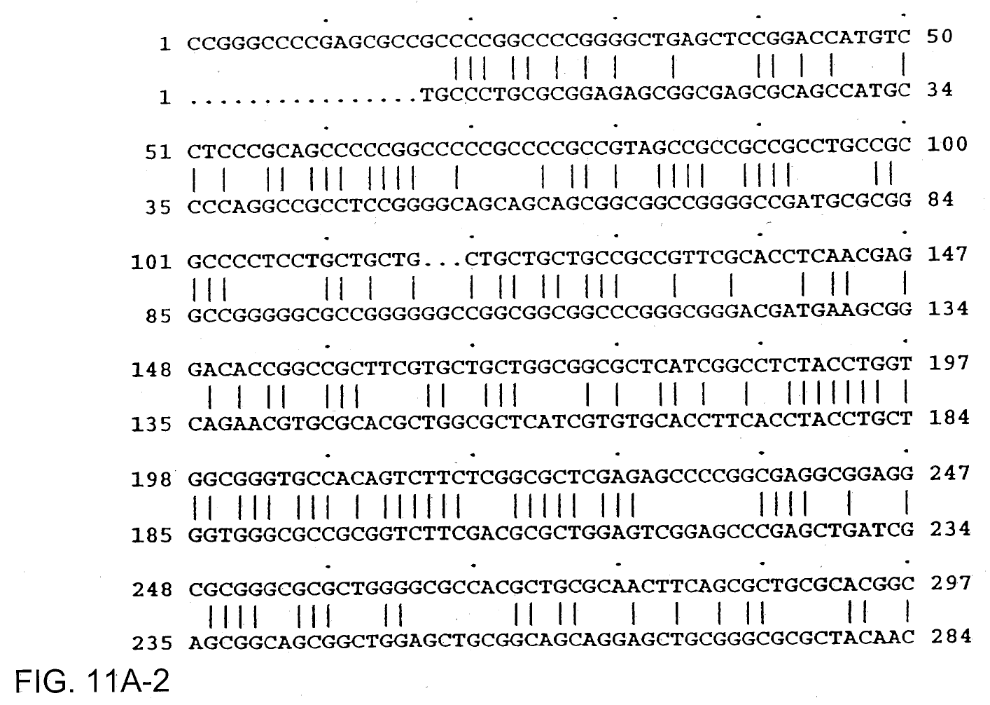

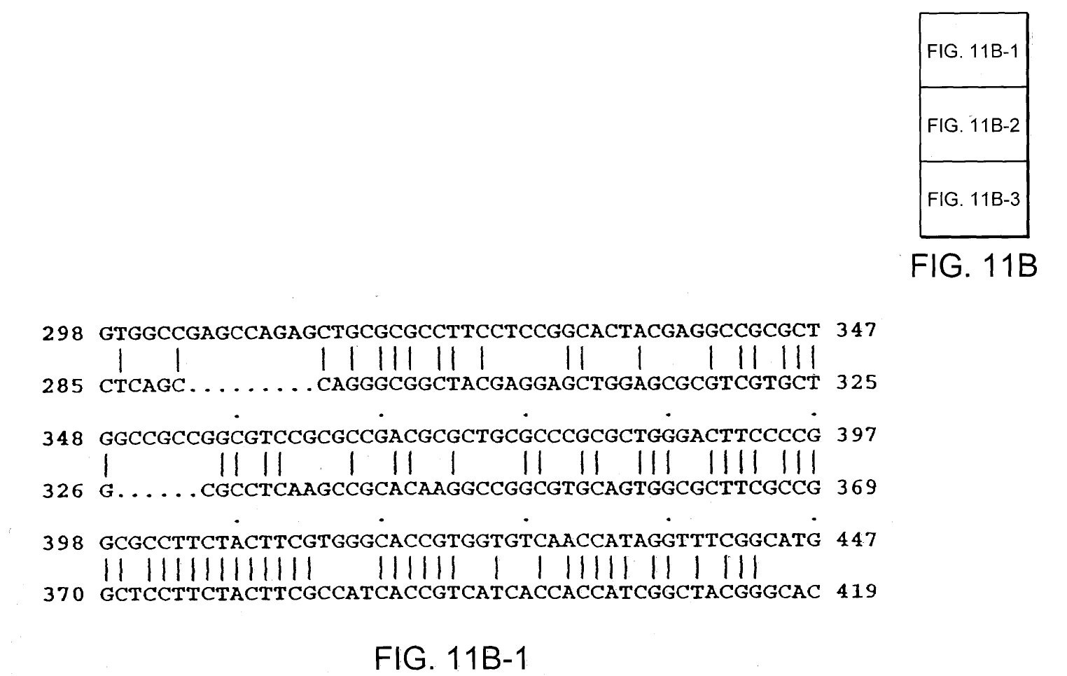

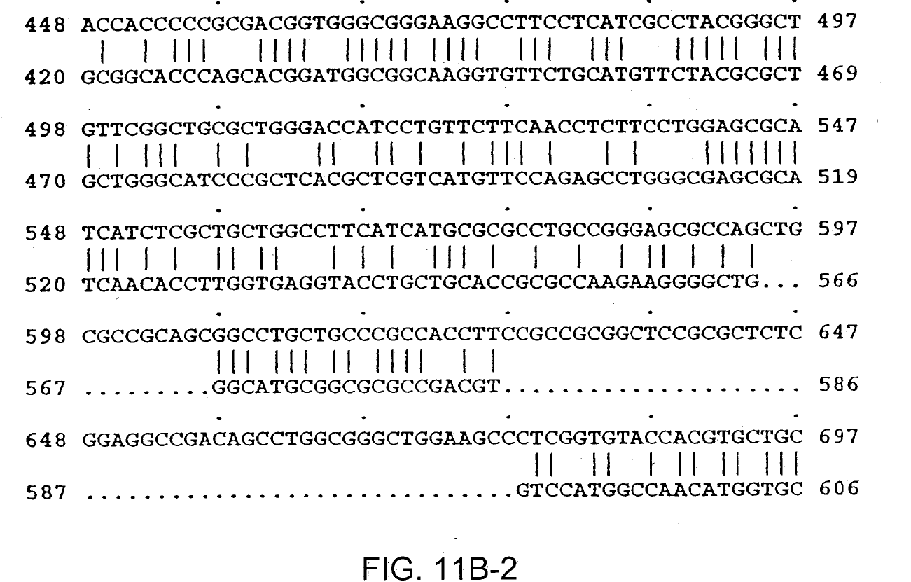

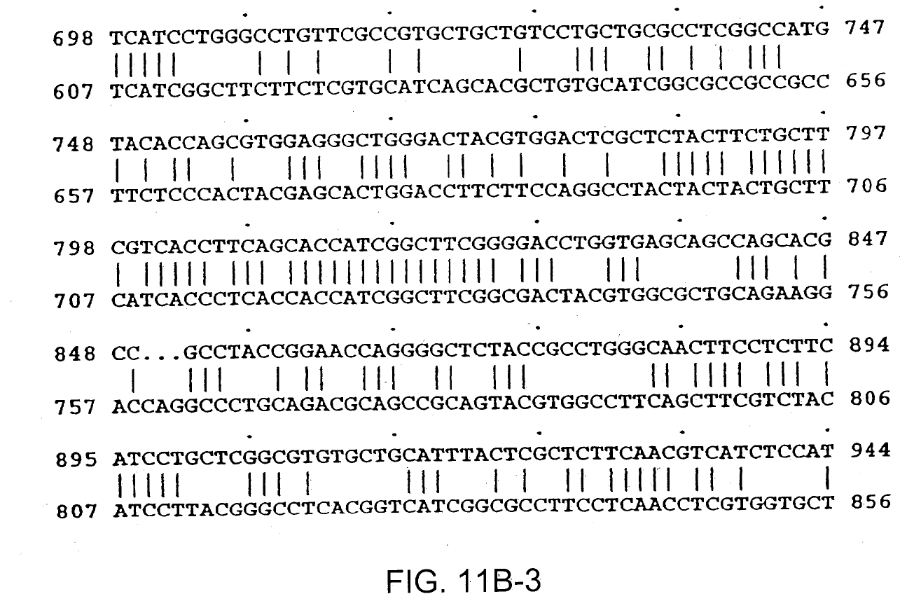

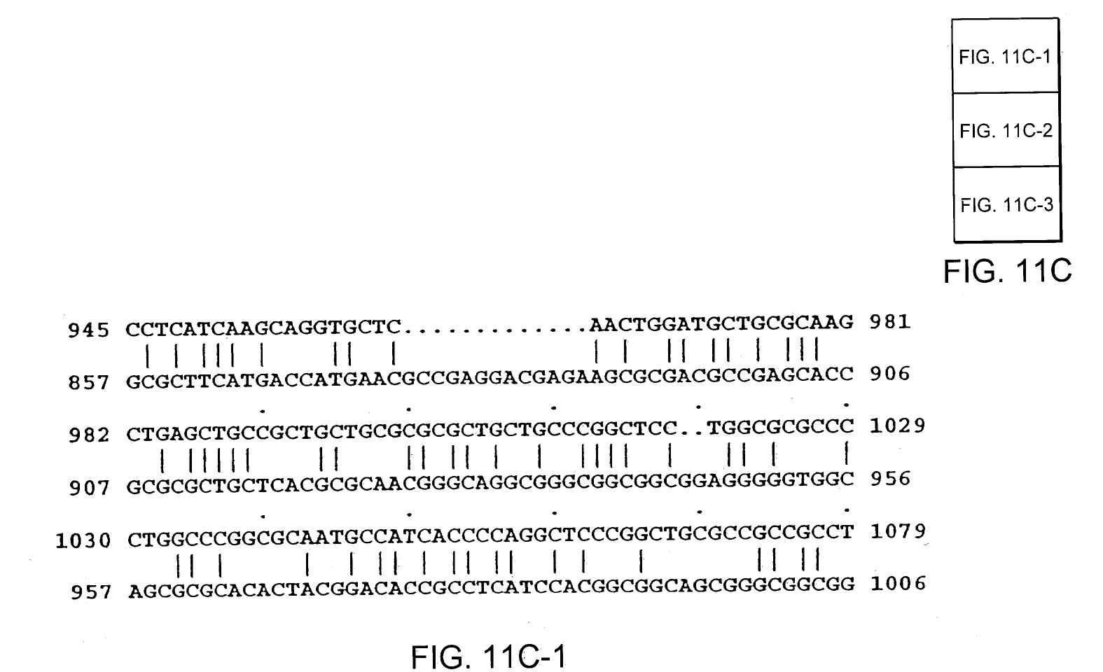

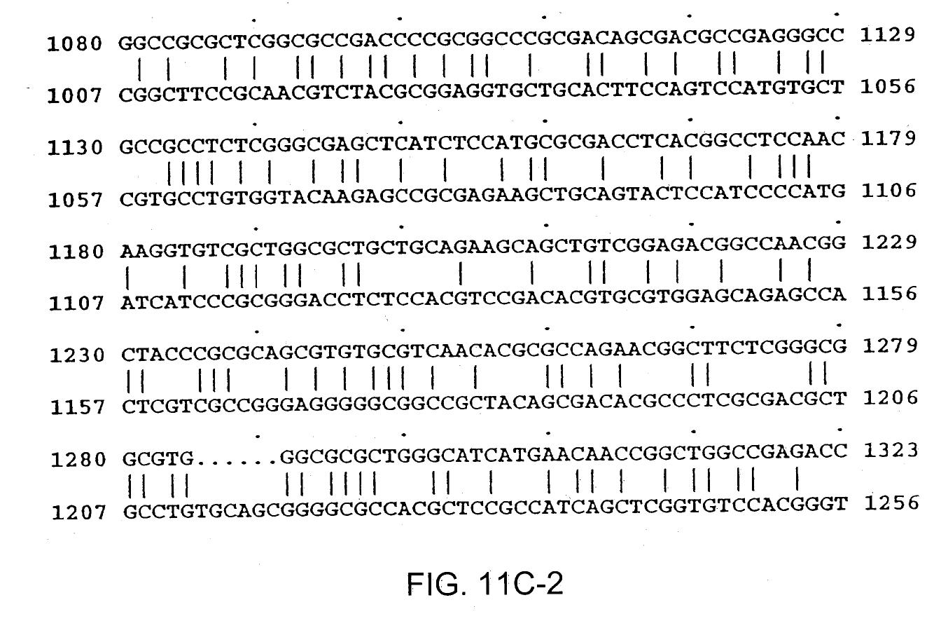

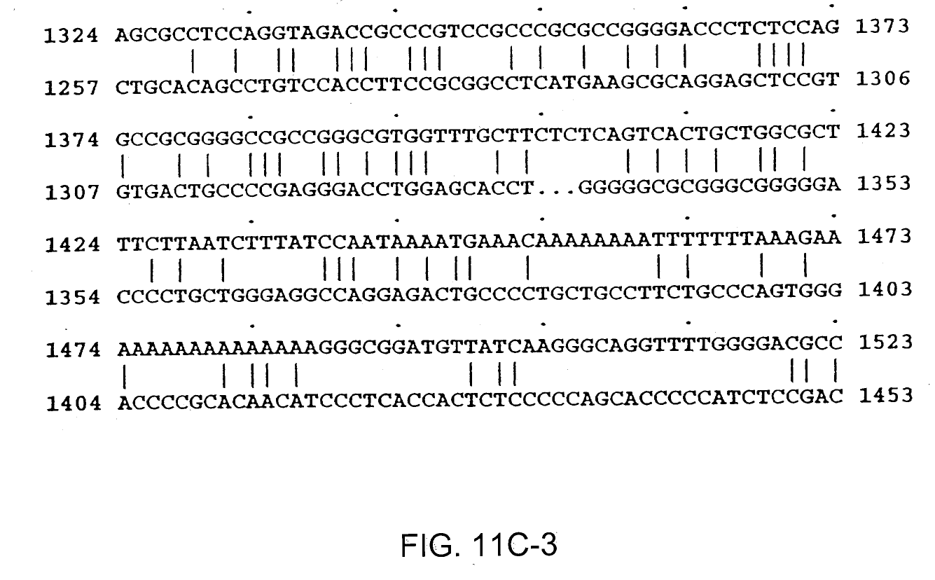

The human TWIK-7 nucleic acid sequence was aligned with the nucleic acid sequence of Homo sapiens TWIK-related acid sensitive K+ channel (TASK) mRNA (Accession Number AF006823), using the GAP program in the GCG software package (nwsgapDNA matrix), a gap weight of 50, and a length weight of 3 (FIGS. 11A-11C). This alignment revealed that human TWIK-7 is 48.317% identical to Accession Number AF006823.

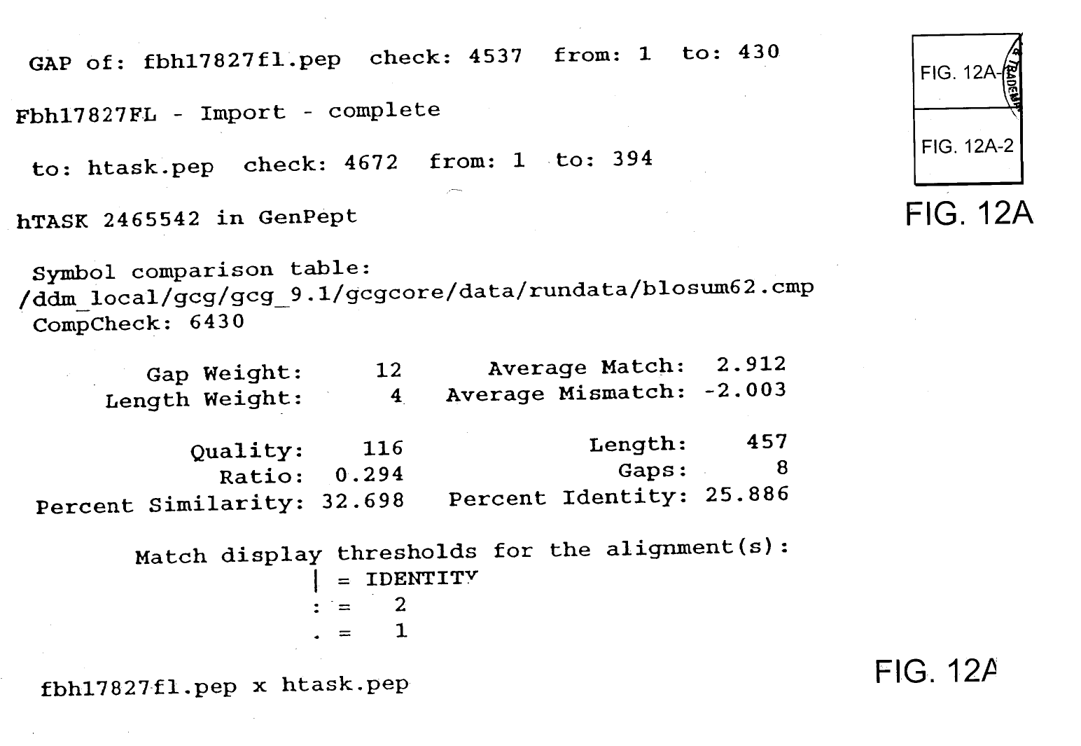

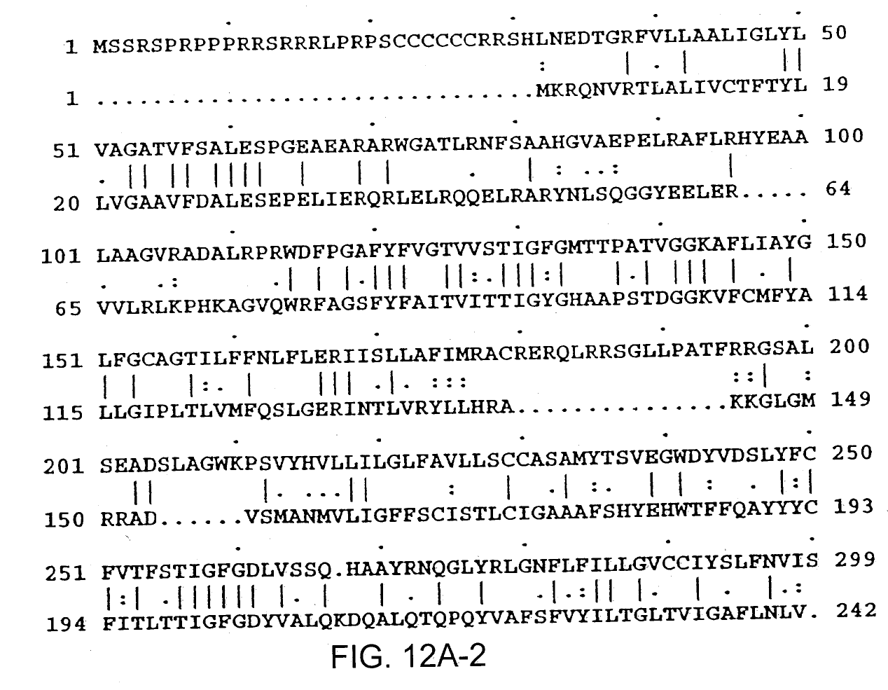

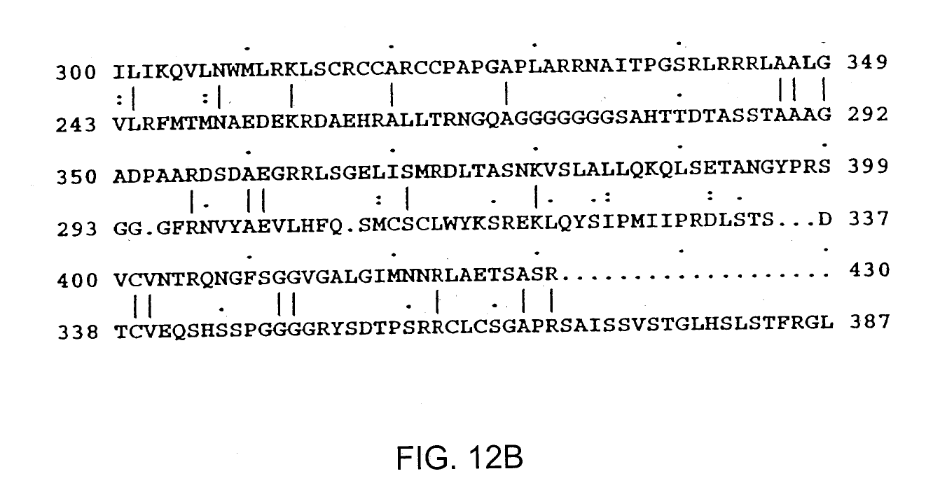

The human TWIK-7 amino acid sequence was aligned with the amino acid sequence of Homo sapiens TWIK related acid sensitive K+ channel (Accession Number AAC51777), using the GAP program in the GCG software package (Blosum 62 matrix), a gap weight of 12, and a length weight of 4 (FIGS. 12A-12B). This alignment revealed that human TWIK-7 is 25.886% identical to Accession Number AAC51777.

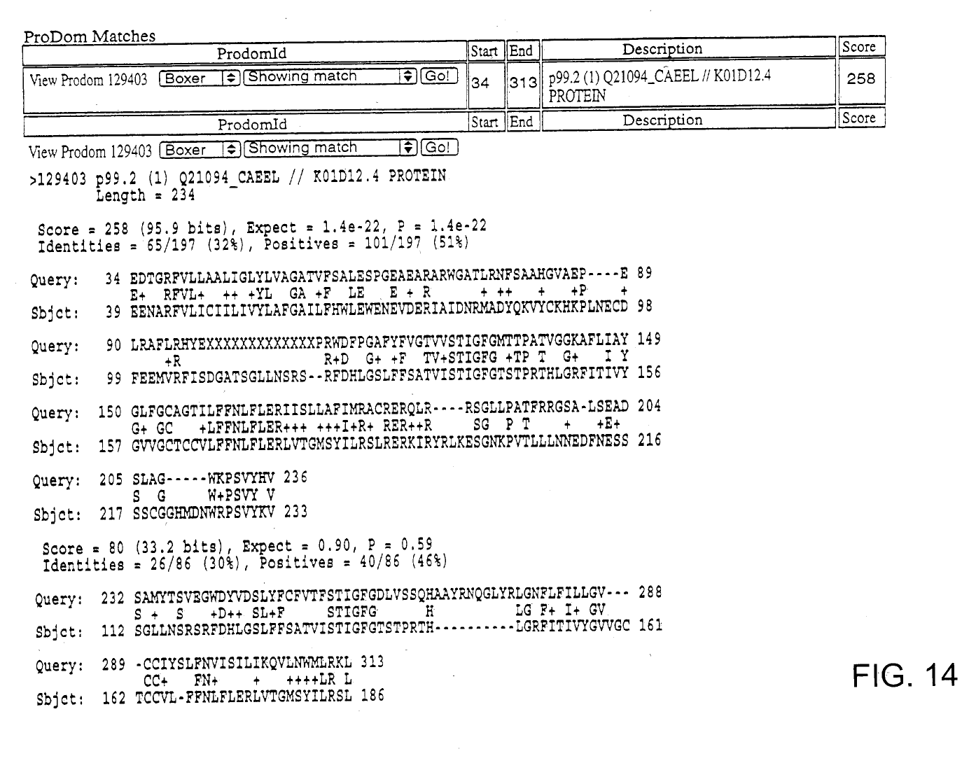

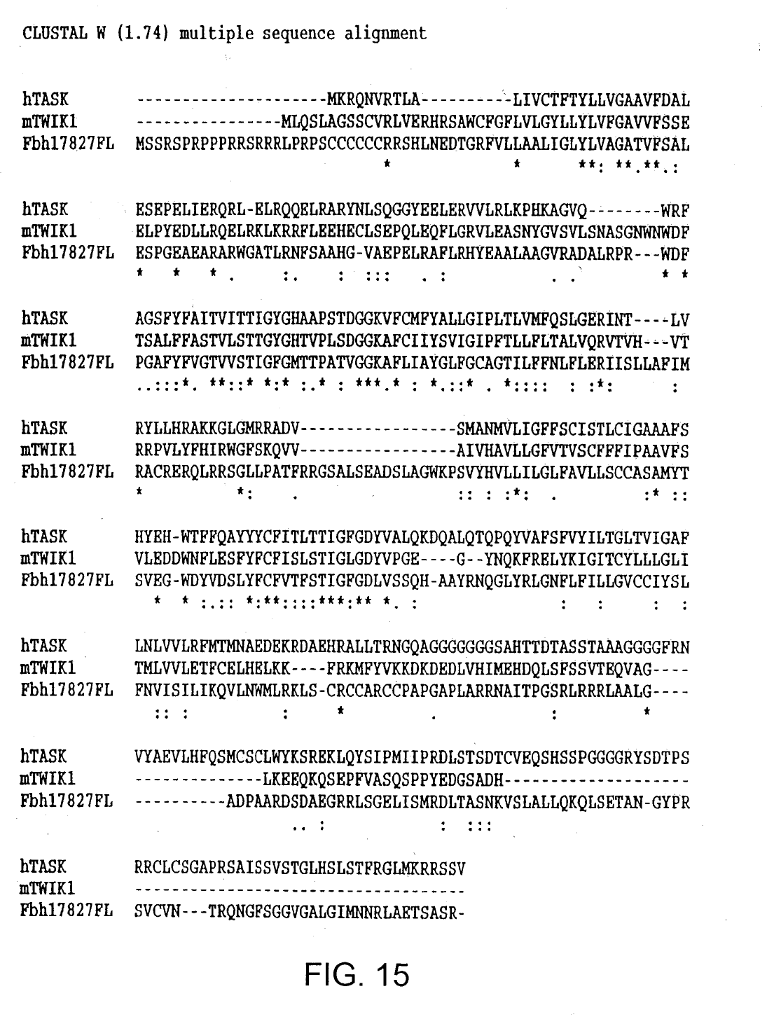

An alignment of the human TWIK-7 amino acid sequence with the amino acid sequences of TWIK-1 from Mus musculus (Accession No. AF006823), and of TWIK-related acid-sensitive K+ channel (Accession No. AF006823) from Homo sapiens, using the CLUSTAL W (1.74) multiple sequence alignment program, is depicted in FIG. 15.

A search was performed against the Memsat database (FIG. 13), resulting in the identification of four transmembrane domains in the amino acid sequence of human TWIK-7 (SEQ ID NO:15) at about residues 39-60, 144-165, 216-235, and 281-302, and two P-loop domains at about residues 119-135 and 247-263.

A search was also performed against the ProDom database resulting in the identification of a potassium channel protein domain in the amino acid sequence of human TWIK-7 (SEQ ID NO:15) at about residues 34-313 (score=258). The results of the search are set forth in FIG. 14.

Tissue Distribution of TWIK-7 mRNA

This example describes the tissue distribution of TWIK-7 mRNA, as was determined by Polymerase Chain Reaction (PCR) on cDNA libraries using oligonucleotide primers based on the human TWIK-7 sequence. The human TWIK-7 gene is strongly expressed in fetal brain, in cerebellum, in T24 bladder carcinoma cells, in A549 lung carcinoma cells, in spinal cord, in fetal dorsal spinal cord, in dorsal root ganglia, in prostate, in uterus, in mammary gland, in normal breast and ovarian epithelia, in spleen, in stomach, in small intestine, and in Th-2 induced T cells. The human TWIK-7 was less strongly expressed in salivary gland, in heart, in thymus, in normal megakaryocytes, in lung, in colon carcinoma, in PTH osteo cells, in erythroblasts, in IBD colon, and in brain subcortical white matter.

For in situ analysis, various tissues, e.g. tissues obtained from brain, are first frozen on dry ice. Ten-micrometer-thick sections of the tissues are postfixed with 4% formaldehyde in DEPC treated 1×phosphate-buffered saline at room temperature for 10 minutes before being rinsed twice in DEPC 1×phosphate-buffered saline and once in 0.1 M triethanolamine-HCl (pH 8.0). Following incubation in 0.25% acetic anhydride-0.1 M triethanolamine-HCl for 10 minutes, sections are rinsed in DEPC 2×SSC (1×SSC is 0.15M NaCl plus 0.015M sodium citrate). Tissue is then dehydrated through a series of ethanol washes, incubated in 100% chloroform for 5 minutes, and then rinsed in 100% ethanol for 1 minute and 95% ethanol for 1 minute and allowed to air dry.

Hybridizations are performed with ³⁵S-radiolabeled (5×10⁷ cpm/ml) cRNA probes. Probes are incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides are washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides are then rinsed with 2×SSC at room temperature, washed with 2×SSC at 50° C. for 1 hour, washed with 0.2×SSC at 55° C. for 1 hour, and 0.2×SSC at 60° C. for 1 hour. Sections are then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

The human TWIK-7 gene is expressed at very high levels in brain. Other organs with lower levels of expression of the human TWIK-7 gene are spinal cord, skin and testis. In situ hybridization experiments showed that human TWIK-7 is expressed in trigeminal (TRG) and dorsal root ganglion (DRG) neurons, in spinal cord and brain. This gene is downregulated in rodent DRG neurons after 2, 7, and 14 days post-axotomy of the sciatic nerve. Expression of human TWIK-7 is also down regulated in DRG neurons in rodent models of inflammatory and neuropathic pain.

### Example 2

### Expression of Recombinant TWIK-7 Protein in Bacterial Cells

In this example, TWIK-7 is expressed as a recombinant glutathione-S-transferase (GST) fusion polypeptide in E. coli and the fusion polypeptide is isolated and characterized. Specifically, TWIK-7 is fused to GST and this fusion polypeptide is expressed in E. coli, e.g, strain PEB199. Expression of the GST-TWIK-7 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant TWIK-7 Protein in COS Cells

To express the TWIK-7 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire TWIK-7 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the TWIK-7 DNA sequence is amplified by PCR using two primers. The 5′ primer contains the restriction site of interest followed by approximately twenty nucleotides of the TWIK-7 coding sequence starting from the initiation codon; the 3′ end sequence contains complementary sequences to the other restriction site of interest, a translation stop codon, the HA tag or FLAG tag and the last 20 nucleotides of the TWIK-7 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the TWIK-7 gene is inserted in the correct orientation. The ligation mixture is transformed into E. coli cells (strains HB101, DH5□, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the TWIK-7-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the TWIK-7 polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the TWIK-7 coding sequence is cloned directly into the polylinker of the pCDNA/Amp vector using the appropriate restriction sites. The resulting plasmid is transfected into COS cells in the manner described above, and the expression of the TWIK-7 polypeptide is detected by radiolabeling and immunoprecipitation using a TWIK-7 specific monoclonal antibody.

### III. 23927, A NOVEL HUMAN ION CHANNEL

### Background of the Invention

The ion channel family of proteins is a large family of membrane-bound proteins responsible for a wide range of important transport and signaling functions in cells. The ion channel family includes at least three subfamilies: calcium ion channels (i.e., Ca channels), potassium channels (i.e., K channels) and sodium channels (Na channels). Members of the ion channel family are characterized by the presence of six (6) transmembrane helices in which the last two helices flank a loop which determines ion selectivity. In some subfamilies (e.g., Na channels) the domain is repeated four times, whereas in others (e.g., K channels) the protein forms as a tetramer in the membrane.

Calcium channel proteins are involved in the control of neurotransmitter release from neurons (Williams et al. (1992) Science 257:389-395), and play an important role in the regulation of a variety of cellular functions, including membrane excitability, muscle contraction and synaptic transmission (Mori et al. (1991) Nature 350:398-402). The calcium channel proteins are composed of four (4) tightly-coupled subunits (α1, α2, β and γ), the α1 subunit from each creating the pore for the import of extracellular calcium ions. The α1 subunit shares sequence characteristics with all voltage-dependent cation channels, and exploits the same 6-helix bundle structural motif. In both sodium and calcium channels, this motif is repeated 4 times within the sequence to give a 24-helix bundle. There are several tissue-specific pharmacologically and electrophysiologically distinct isoforms of calcium channels, coded for by separate genes in a multi-gene family. In skeletal muscle, each tightly-bound assembly of α, β and γ subunits associates with 4 others to form a pentameric macromolecule (Koch et al. (1990) J. Biol. Chem. 265:17786-17791). Examples of calcium channels include, but are not limited to, the low-voltage-gated channels and the high-voltage-gated channels. Calcium channels are described in, for example, Davila et al. (1999) Annals New York Acad. Sci. 868:102-17 and McEnery et al. (1998) J. Bioenergetics and Biomembranes 30(4):409-418, the contents of which are incorporated herein by reference.

Sodium channels are transmembrane (TM) voltage-dependent proteins responsible for the depolarizing phase of the action potential in most electrically excitable cells (George et al. (1992) Proc. Natl. Acad. Sci. USA 89:4893-4897). They may exist in 3 states (Noda et al. (1984) Nature 312:121-127): the resting state, where the channel is closed; the activated state, where the channel is open; and the inactivated state, where the channel is closed. Several different structurally and functionally distinct isoforms are found in mammals, coded for by a multigene family (Rogart et al. (1989) Proc. Natl. Acad. Sci. USA 86:8170-8174), these being responsible for the different types of sodium ion currents found in excitable tissues. The structure of sodium channels is based on 4 internal repeats of a 6-helix bundle (Noda et al. (1986) Nature 320:188-192) (in which 5 of the membrane-spanning segments are hydrophobic and the other is positively charged), forming a 24-helical bundle. The charged segments are believed to be localized within clusters formed by their 5 hydrophobic neighbors. It is postulated that the charged domain may be the voltage sensor region, possibly moving outward on depolarization, causing a conformational change. This model, proposed by (Noda et al., supra), contrasts with that of Sato and Matsumoto (1992) Biochem. Biophys. Res. Commun.. 186:1158-1167), in which the TM segments are juxtaposed octagonally. The basic structural motif (the 6-helix bundle) is also found in potassium and calcium channels.

Potassium channels are the most diverse group of the ion channel family (possibly as a result of gene duplication and alternative splicing of the genes (Perney and Kaczmarek (1991) Curr. Opin. Cell. Biol. 3:663-670 and Luneau et al. (1991) FEBS Lett. 288:163-167). They are important in shaping the action potential, and in neuronal excitability and plasticity (Tempel et al. (1988) Nature 332:837-839). The potassium channel family is composed of several functionally distinct isoforms, which can be broadly separated into 2 groups (Stuehmer et al. (1989) EMBO J. 8:3225-3244). The first is the practically non-inactivating “delayed” group, the second the rapidly inactivating “transient” group. These are all highly similar proteins, with possibly only small amino acid changes causing the diversity of the voltage-dependent gating mechanism, channel conductance and toxin binding properties. Members of the potassium channel family vary in several ways. Some open in response to depolarization of the plasma membrane; others open in response to hyperpolarization or an increase in intracellular calcium concentration; some can be regulated by binding of a transmitter, together with intracellular kinases; and others are regulated by GTP-binding proteins or other second messengers (Schwarz et al. (1988) Nature 331:137-142 (1988). They are also involved in T-cell activation, and may have a role in target cell lysis by cytotoxic T-lymphocytes (Attali et al. (1992) J. Biol. Chem. 267:8650-8657 (1992). Potassium channels are transmembrane (TM) proteins that contain 6 membrane-spanning α-helical segments, 5 of which are hydrophobic, the other being positively charged. The charged segment is believed to be localized within a cluster formed by the hydrophobic helices. As with Na channels, it is postulated that the charged segment may constitute the voltage sensor region, possibly moving outward on depolarization, causing a conformational change. The 6-helix bundle is a common structural motif in sodium channels (in which it is repeated 4 times within the sequence to form a 24-helix bundle), and in calcium channels (where it also forms a 24-helix bundle, which itself is tightly bound to 3 different subunits).

Ion channels play a role in regulating ion transport and signaling in virtually every cell in the human body. This fundamental knowledge has fostered an extensive search for modulators of such receptors for use as human therapeutics. In fact, the ion channel superfamily has proven to be among the most successful drug targets. Consequently, it has been recognized that newly isolated ion channels have great potential for drug discovery (e.g., for identifying modulators of such channels for use in regulating a variety of cellular responses.

### Summary of the Invention

The present invention is based, at least in part, on the discovery of novel ion channel family members, referred to herein as ion channel 23927 or IC23927 or 23927 nucleic acid and protein molecules. The IC23927 molecules of the present invention are useful as targets for developing modulating agents to regulate a variety of cellular processes, including ion transport (e.g., ion conductance), membrane excitability and/or polarization, synaptic transmission, signal transduction (e.g., pain signaling), cell activation, proliferation, growth, differentiation and/or migration as well as muscle contraction. The 23927 nucleic acid and protein molecules (and modulators thereof) are also useful in regulating physiologic processes, for example, pain and/or pain disorders. Accordingly, in one aspect, this invention provides isolated nucleic acid molecules encoding IC23927 proteins or biologically active portions thereof, as well as nucleic acid fragments suitable as primers or hybridization probes for the detection of IC23927-encoding nucleic acids.

In one embodiment, an IC23927 nucleic acid molecule of the invention is at least 50%, 55%, 60%, 65%, 70%, 75%, 80%, 85%, 90%, 95%, 96%, 97%, 98%, 99% identical to the nucleotide sequence (e.g., to the entire length of the nucleotide sequence) shown in SEQ ID NO:21 or 23 or the nucleotide sequence of the DNA insert of the plasmid deposited with ATCC as Accession Number ______, or a complement thereof.

In a preferred embodiment, the isolated nucleic acid molecule includes the nucleotide sequence shown SEQ ID NO:21 or 23, or a complement thereof. In another embodiment, the nucleic acid molecule includes SEQ ID NO:23 and nucleotides 1-287 of SEQ ID NO:21. In yet another embodiment, the nucleic acid molecule includes SEQ ID NO:23 and nucleotides 2339-5269 of SEQ ID NO:21. In another preferred embodiment, the nucleic acid molecule consists of the nucleotide sequence shown in SEQ ID NO:21 or 23. In another preferred embodiment, the nucleic acid molecule includes a fragment of at least 2451 nucleotides (e.g., 2451 contiguous nucleotides) of the nucleotide sequence of SEQ ID NO:21 or 23, or a complement thereof.

In another embodiment, an IC23927 nucleic acid molecule includes a nucleotide sequence encoding a protein having an amino acid sequence sufficiently identical to the amino acid sequence of SEQ ID NO:22 or an amino acid sequence encoded by the DNA insert of the plasmid deposited with ATCC as Accession Number ______. In a preferred embodiment, an IC23927 nucleic acid molecule includes a nucleotide sequence encoding a protein having an amino acid sequence at least 50%, 55%, 60%, 65%, 70%, 75%, 80%, 85%, 90%, 95%, 96%, 97%, 98%, 99% or more identical to the entire length of the amino acid sequence of SEQ ID NO:22 or the amino acid sequence encoded by the DNA insert of the plasmid deposited with ATCC as Accession Number ______.

In another preferred embodiment, an isolated nucleic acid molecule encodes the amino acid sequence of human IC23927. In yet another preferred embodiment, the nucleic acid molecule includes a nucleotide sequence encoding a protein having the amino acid sequence of SEQ ID NO:22, or the amino acid sequence encoded by the DNA insert of the plasmid deposited with ATCC as Accession Number ______. In yet another preferred embodiment, the nucleic acid molecule is at least 2451 nucleotides in length. In a further preferred embodiment, the nucleic acid molecule is at least 2393 nucleotides in length and encodes a protein having an IC23927 activity (as described herein).

Another embodiment of the invention features nucleic acid molecules, preferably IC23927 nucleic acid molecules, which specifically detect IC23927 nucleic acid molecules relative to nucleic acid molecules encoding non-IC23927 proteins. For example, in one embodiment, such a nucleic acid molecule is at least 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2200-2300, 2300-2400, 2400-2500 or more nucleotides in length and hybridizes under stringent conditions to a nucleic acid molecule comprising the nucleotide sequence shown in SEQ ID NO:21, the nucleotide sequence of the DNA insert of the plasmid deposited with ATCC as Accession Number ______, or a complement thereof.

In other preferred embodiments, the nucleic acid molecule encodes a naturally occurring allelic variant of a polypeptide comprising the amino acid sequence of SEQ ID NO:22 or an amino acid sequence encoded by the DNA insert of the plasmid deposited with ATCC as Accession Number ______, wherein the nucleic acid molecule hybridizes to a nucleic acid molecule comprising SEQ ID NO:21 or 23 under stringent conditions.

Another embodiment of the invention provides an isolated nucleic acid molecule which is antisense to an IC23927 nucleic acid molecule, e.g., the coding strand of an IC23927 nucleic acid molecule.

Another aspect of the invention provides a vector comprising an IC23927 nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably an IC23927 protein, by culturing in a suitable medium, a host cell, e.g., a mammalian host cell such as a non-human mammalian cell, of the invention containing a recombinant expression vector, such that the protein is produced.

Another aspect of this invention features isolated or recombinant IC23927 proteins and polypeptides. In one embodiment, an isolated IC23927 protein includes at least one transmembrane domain. In another embodiment, an isolated IC23927 protein includes at least one ion transport protein (ITP) domain. In another embodiment, an isolated IC23927 protein includes at least one site selected from the group consisting of an N-glycosylation site, a protein kinase C phosphorylation site, a casein kinase II phosphorylation site, a tyrosine kinase phosphorylation site, an N-myristoylation site and an amidation site. In yet another embodiment, an isolated IC23927 protein includes at least one transmembrane domain and/or an ITP domain and at least one site selected from the group consisting of an N-glycosylation site, a protein kinase C phosphorylation site, a casein kinase II phosphorylation site, a tyrosine kinase phosphorylation site, an N-myristoylation site and an amidation site.

In a preferred embodiment, an IC23927 protein includes at least one transmembrane domain and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:22, or the amino acid sequence encoded by the DNA insert of the plasmid deposited with ATCC as Accession Number ______. In a further preferred embodiment, an IC23927 protein includes an ITP domain and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:22, or the amino acid sequence encoded by the DNA insert of the plasmid deposited with ATCC as Accession Number ______. In a further preferred embodiment, an IC23927 protein includes at least one transmembrane domain and/or an ITP domain and at least one site selected from the group consisting of an N-glycosylation site, a protein kinase C phosphorylation site, a casein kinase II phosphorylation site, a tyrosine kinase phosphorylation site, an N-myristoylation site and an amidation site and has an amino acid sequence at least about 50%, 55%, 60%, 65%, 70%, 75%, 80%, 85%, 90%, 95%, 96%, 97%, 98%, 99% or more identical to the amino acid sequence of SEQ ID NO:22, or the amino acid sequence encoded by the DNA insert of the plasmid deposited with ATCC as Accession Number ______.

In another preferred embodiment, an IC23927 protein includes at least one transmembrane domain and/or an ITP domain and has an IC23927 activity (as described herein).

In yet another preferred embodiment, an IC23927 protein includes at least one transmembrane domain and is encoded by a nucleic acid molecule having a nucleotide sequence which hybridizes under stringent hybridization conditions to a nucleic acid molecule comprising the nucleotide sequence of SEQ ID NO:21 or 23. In a further embodiment, an IC23927 protein includes an ITP domain and is encoded by a nucleic acid molecule having a nucleotide sequence which hybridizes under stringent hybridization conditions to a nucleic acid molecule comprising the nucleotide sequence of SEQ ID NO:21 or 23. In another embodiment, an IC23927 protein includes at least one transmembrane and/or an ITP domain and at least one site selected from the group consisting of an N-glycosylation site, a protein kinase C phosphorylation site, a casein kinase II phosphorylation site, a tyrosine kinase phosphorylation site, an N-myristoylation site and an amidation site and is encoded by a nucleic acid molecule having a nucleotide sequence which hybridizes under stringent hybridization conditions to a nucleic acid molecule comprising the nucleotide sequence of SEQ ID NO:21 or 23.

In another embodiment, the invention features fragments of the protein having the amino acid sequence of SEQ ID NO:22, wherein the fragment comprises at least 15, 50, 100, 150, 200, 250, 300, 350, 400, 450, 500, 550, 600, 650, 700, 750, 800 or more amino acids (e.g., contiguous amino acids) of the amino acid sequence of SEQ ID NO:22, or an amino acid sequence encoded by the DNA insert of the plasmid deposited with the ATCC as Accession Number ______. In another embodiment, an IC23927 protein has the amino acid sequence of SEQ ID NO:22.

In another embodiment, the invention features an IC23927 protein which is encoded by a nucleic acid molecule consisting of a nucleotide sequence at least about 50%, 55%, 60%, 65%, 70%, 75%, 80%, 85%, 90%, 95%, 96%, 97%, 98%, 99% or more identical to a nucleotide sequence of SEQ ID NO:21 or 23, or a complement thereof. This invention further features an IC23927 protein, which is encoded by a nucleic acid molecule consisting of a nucleotide sequence which hybridizes under stringent hybridization conditions to a nucleic acid molecule comprising the nucleotide sequence of SEQ ID NO:21 or 23, or a complement thereof.

The proteins of the present invention or portions thereof, e.g., biologically active portions thereof, can be operatively linked to a non-IC23927 polypeptide (e.g., heterologous amino acid sequences) to form fusion proteins. The invention further features antibodies, such as monoclonal or polyclonal antibodies, that specifically bind proteins of the invention, preferably IC23927 proteins. In addition, the IC23927 proteins or biologically active portions thereof can be incorporated into pharmaceutical compositions, which optionally include pharmaceutically acceptable carriers.

In another aspect, the present invention provides a method for detecting the presence of an IC23927 nucleic acid molecule, protein, or polypeptide in a biological sample by contacting the biological sample with an agent capable of detecting an IC23927 nucleic acid molecule, protein, or polypeptide such that the presence of an IC23927 nucleic acid molecule, protein or polypeptide is detected in the biological sample.

In another aspect, the present invention provides a method for detecting the presence of IC23927 activity in a biological sample by contacting the biological sample with an agent capable of detecting an indicator of IC23927 activity such that the presence of IC23927 activity is detected in the biological sample.

In another aspect, the invention provides a method for modulating IC23927 activity comprising contacting a cell capable of expressing IC23927 with an agent that modulates IC23927 activity such that IC23927 activity in the cell is modulated. In one embodiment, the agent inhibits IC23927 activity. In another embodiment, the agent stimulates IC23927 activity. In one embodiment, the agent is an antibody that specifically binds to an IC23927 protein. In another embodiment, the agent modulates expression of IC23927 by modulating transcription of an IC23927 gene or translation of an IC23927 mRNA. In yet another embodiment, the agent is a nucleic acid molecule having a nucleotide sequence that is antisense to the coding strand of an IC23927 mRNA or an IC23927 gene.

In one embodiment, the methods of the present invention are used to treat a subject having a disorder characterized by aberrant or unwanted IC23927 protein or nucleic acid expression or activity by administering an agent which is an IC23927 modulator to the subject. In one embodiment, the IC23927 modulator is an IC23927 protein. In another embodiment the IC23927 modulator is an IC23927 nucleic acid molecule. In yet another embodiment, the IC23927 modulator is a peptide, peptidomimetic, or other small molecule. In a preferred embodiment, the disorder characterized by aberrant or unwanted IC23927 protein or nucleic acid expression is a CNS disorder, such as a neurodegenerative disorder, (e.g., Alzheimer's disease), Parkinson's disease, multiple sclerosis, amyotrophic lateral sclerosis, AIDS related dementia, a convulsion, palsy or epilepsy, a psychiatric disorder (e.g., depression, schizophrenic disorders, mania, anxiety disorders, or phobic disorders) or a learning or memory disorder (e.g., amnesia or age-related memory loss) or is a neurological disorder (e.g., migraine). In another embodiment, the disorder characterized by aberrant or unwanted IC23927 activity is a pain disorder (e.g., a disorder characterized by misregulated pain signaling mechanisms). In another embodiment, the disorder characterized by aberrant or unwanted IC23927 activity is a cell activation, proliferation, growth, differentiation, or migration disorder.

The present invention also provides diagnostic assays for identifying the presence or absence of a genetic alteration characterized by at least one of (i) aberrant modification or mutation of a gene encoding an IC23927 protein; (ii) mis-regulation of the gene; and (iii) aberrant post-translational modification of an IC23927 protein, wherein a wild-type form of the gene encodes a protein with an IC23927 activity.

In another aspect the invention provides methods for identifying a compound that binds to or modulates the activity of an IC23927 protein, by providing an indicator composition comprising an IC23927 protein having IC23927 activity, contacting the indicator composition with a test compound, and determining the effect of the test compound on IC23927 activity in the indicator composition to identify a compound that modulates the activity of an IC23927 protein.

In a further aspect, the invention provides a method for identifying a compound which modulates pain comprising contacting a IC23927 polypeptide or a cell which expresses a IC23927 polypeptide with a test compound with a test compound and identifying the compound as a modulator of pain by determining the effect of the test compound on the activity of the polypeptide. In yet another aspect, the invention provides a method for identifying a compound capable of modulating nociception comprising contacting a IC23927 polypeptide or a cell which expresses a IC23927 polypeptide with a test compound and identifying the compound as a modulator of nociception by determining the effect of the test compound on the activity of the polypeptide.

The present invention further features a method for treating a subject having pain or a pain disorder comprising administering to the subject a IC23927 modulator. In one embodiment, the IC23927 modulator is a small molecule. In another embodiment, the IC23927 modulator is administered in a pharmaceutically acceptable formulation. In yet another embodiment the IC23927 modulator is administered using a gene therapy vector.

Other features and advantages of the invention will be apparent from the following detailed description and claims.

### Detailed Description of the Invention

The present invention is based, at least in part, on the discovery of novel molecules, referred to herein as ion channel 23927 or “IC23927” nucleic acid and protein molecules, which are novel members of the ion channel family of proteins and nucleic acid molecules. These novel molecules are capable of, for example, modulating ion transport in a cell (e.g., a neuronal, muscle (e.g., cardiac muscle), or liver cell).

As used herein, an “ion channel” includes a protein or polypeptide which is involved in receiving, conducting, and transmitting signals in an cell (e.g., an electrically excitable cell, for example, a neuronal or muscle cell). Ion channels can determine membrane excitability (the ability of, for example, a cell to respond to a stimulus and to convert it into a sensory impulse). Ion channels can also influence the resting potential of membranes, wave forms and frequencies of action potentials, and thresholds of excitation. Ion channels are typically expressed in electrically excitable cells, e.g., neuronal cells, and may form heteromultimeric structures (e.g,, composed of more than one type of subunit). Ion channels may also be found in nonexcitable cells (e.g., adipose cells or liver cells), where they may play a role in, for example, signal transduction. As the IC23927 molecules of the present invention may modulate ion channel mediated activities, they may be useful for developing novel diagnostic and therapeutic agents for ion channel associated disorders.

As used herein, an “ion channel associated disorder” includes a disorder, disease or condition which is characterized by a misregulation of ion channel mediated activity. Ion channel associated disorders include CNS disorders, such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, Jakob-Creutzfieldt disease, or AIDS related dementia; autonomic function disorders such as hypertension and sleep disorders, and neuropsychiatric disorders, such as depression, schizophrenia, schizoaffective disorder, korsakoff's psychosis, mania, anxiety disorders, or phobic disorders; leaning or memory disorders, e.g., amnesia or age-related memory loss, attention deficit disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Ion channel disorders also include pain disorders. As used herein, the term “pain disorder” includes disorders characterized by aberrant (e.g., excessive or amplified) pain signaling in addition to symptoms and/or phenotypes which result from wild-type, or normal, pain signaling mechanisms. Examples of pain disorders include posttherapeutic neuralgia, diabetic neuropathy, postmastectomy pain syndrome, stump pain, reflex sympathetic dystrophy, trigeminal neuralgia, neuropathic pain, orofacial neuropathic pain, osteoarthritis, rheumatoid arthritis, fibromyalgia syndrome, tension myalgia, Guillian-Barre syndrome, Meralgia paraesthetica, burning mouth syndrome, fibrocitis, myofascial pain syndrome, idiopathic pain disorder, temporomandibular joint syndrome, atypical odontalgia, loin pain, haematuria syndrome, non-cardiac chest pain, low back pain, chronic nonspecific pain, psychogenic pain, musculoskeletal pain disorder, chronic pelvic pain, nonorganic chronic headache, tension-type headache, cluster headache, migraine, complex regional pain syndrome, vaginismus, nerve trunk pain, somatoform pain disorder, cyclical mastalgia, chronic fatigue syndrome, multiple somatization syndrome, chronic pain disorder, somatization disorder, Syndrome X, facial pain, idiopathic pain disorder, posttraumatic rheumatic pain modulation disorder (fibrositis syndrome), hyperalgesia, and Tangier disease. As used herein, the term “pain signaling mechanisms” includes the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter sensory neurons. These sensory neurons transmit the information to the central nervous system, evoking a perception of pain or discomfort and initiating appropriate protective reflexes. The IC23927 molecules of the present invention may be present on sensory neurons and, thus, may be involved in detecting, for example, noxious chemical, mechanical, or thermal stimuli and transducing this information into membrane depolarization events. Thus, the IC23927 molecules by participating in pain signaling mechanisms, may modulate pain elicitation and act as targets for developing novel diagnostic targets and therapeutic agents to control pain. Moreover, IC23927 mRNA is predominantly expressed in the brain as compared to other tissues evidencing the usefulness of the IC23927 molecules of the present invention as targets for developing novel diagnostic targets and therapeutic agents to control pain and pain disorders.

Ion channel disorders also include cellular activation, proliferation, growth, differentiation, or migration disorders. Cellular activation, proliferation, growth, differentiation, or migration disorders include those disorders that affect cell activation, proliferation, growth, differentiation, or migration processes. As used herein, a “cellular activation, proliferation, growth, differentiation, or migration process” is a process by which a cell increases in activity (e.g., a cell-specific activity), number, size or content, by which a cell develops a specialized set of characteristics which differ from that of other cells, or by which a cell moves closer to or further from a particular location or stimulus. The IC23927 molecules of the present invention are also involved in signal transduction mechanisms, which are known to be involved in cellular activation, growth, differentiation, and migration processes. Thus, the IC23927 molecules may modulate activation, cellular growth, differentiation, or migration, and may play a role in disorders characterized by aberrantly regulated activation, growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; hepatic disorders; cardiovascular disorders; and hematopoietic and/or myeloproliferative disorders.

As used herein, an “ion channel mediated activity” includes an activity which involves an ion channel, e.g., a calcium channel in a neuronal cell, a muscular cell, or a liver cell, associated with receiving, conducting, and transmitting signals, in, for example, the nervous system. Ion channel mediated activities include release of neurotransmitters or second messenger molecules (e.g., dopamine or norepinephrine), from cells, e.g., neuronal cells; modulation of resting potential of membranes, wave forms and frequencies of action potentials, and thresholds of excitation; participation in signal transduction pathways, and modulation of processes such as integration of sub-threshold synaptic responses and the conductance of back-propagating action potentials in, for example, neuronal cells (e.g., changes in those action potentials resulting in a morphological or differentiative response in the cell).

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., mouse proteins. Members of a family may also have common functional characteristics.

For example, the family of IC23927 proteins comprise at least one “transmembrane domain” and preferably six, seven, eight, nine, ten, eleven or twelve transmembrane domains. As used herein, the term “transmembrane domain” includes an amino acid sequence of about 10-45 amino acid residues in length which spans the plasma membrane. More preferably, a transmembrane domain includes about at least 12, 15, 20, 25, 30, 35 or 40 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta et al. (1996) Ann. Rev. Neurosci. 19: 235-263, the contents of which are incorporated herein by reference. Amino acid residues 114-128, 146-168, 178-195, 199-210, 233-254, 298-320, 445-465, 482-502, 510-532, 539-554, 570-594 and 666-687 of the IC23927 protein comprise transmembrane domains (see FIG. 17). Accordingly, IC23927 proteins having at least 50-60% homology, preferably about 60-70%, more preferably about 70-80%, or about 80-90% homology with a transmembrane domain of human IC23927 are within the scope of the invention.

In another embodiment, an IC23927 molecule of the present invention is identified based on the presence of at least one pore domain between the fifth and sixth transmembrane domains. As used herein, the term “pore domain” includes an overall hydrophobic amino acid sequence which is located between two transmembrane domains of an ion channel protein, preferably transmembrane domains 5 and 6, and which is believed to be a major determinant of ion selectivity and activity in ion channels. Pore domains are described, for example in Vannier et al. (1998) J. Biol. Chem. 273: 8675-8679 and Phillips, A. M. et al. (1992) Neuron 8, 631-642, the contents of which are incorporated herein by reference. Pore domains in the human IC23927 amino acid sequence can be found, for example, from amino acids 269-287 of SEQ ID NO:22 (P1) and from amino acid 637-653 of SEQ ID NO:22 (P2). IC23927 molecules having at least one pore domain are within the scope of the invention.

In another embodiment, an IC23927 molecule of the present invention is identified based on the presence of at least one N-glycosylation site. As used herein, the term “N-glycosylation site” includes an amino acid sequence of about 4 amino acid residues in length which serves as a potential N-glycosylation site. Preferably, an N-glycosylation site has the consensus sequence N-X-[ST] (where X is any amino acid and [ST] is serine or threonine). More preferably, an N-glycosylation site has the consensus sequence N-{P}-[ST]-{P} (where N is a glycosylation site, {P} is any amino acid sequence but proline and [ST] is serine or threonine). (All consensus sequences used herein are written according to art-recognized designations of amino acid residues). N-glycosylation sites are described in, for example, Prosite PDOC00001 (http://www.expasy.ch/cgi-bin/nicedoc.p1?PDOC00001), the contents of which are incorporated herein by reference. Amino acid residues 599-602, 611 -614, 616-619 and 695-698 of the IC23927 protein comprise N-glycosylation sites. Accordingly, IC23927 proteins having at least one N-glycosylation site are within the scope of the invention.

In another embodiment, an IC23927 molecule of the present invention is identified based on the presence of at least one protein kinase C (PKC) phosphorylation site. As used herein, the term “PKC phosphorylation site” includes an amino acid sequence of about 3 amino acid residues in length which includes a serine or threonine residue which is potentially phosphorylated by protein kinase C (PKC). Preferably, a PKC phosphorylation site has the consensus sequence [ST]-X-[RK] [where S or T is the phosphorylation site, X is any amino acid and DE is aspartate or glutamate]. PKC phosphorylation sites are described in, for example, Prosite PDOC00001 (http://www.expasy.ch/cgi-bin/nicedoc.p1?PDOC00005), the contents of which are incorporated herein by reference. Amino acid residues 351-353, 359-361, 375-377, 382-384, 395-397, 697-699 and 769-771 of the IC23927 protein comprise PKC phosphorylation sites. Accordingly, IC23927 proteins having at least one PKC phosphorylation site are within the scope of the invention.

In another embodiment, an IC23927 molecule of the present invention is identified based on the presence of at least one casein kinase II phosphorylation site. As used herein, the term “casein kinase II phosphorylation site” includes an amino acid sequence of about 4 amino acid residues in length which includes a serine or threonine residue which is potentially phosphorylated by casein kinase II (CK-2). Preferably, a casein kinase II phosphorylation site has the consensus sequence [ST]-X(2)-[DE] [where S or T is the phosphorylation site and X is any amino acid]. Casein kinase II phosphorylation sites are described in, for example, Prosite PDOC00006 (http://www.expasy.ch/cgi-bin/nicedoc.p1?PDOC00006), the contents of which are incorporated herein by reference. Amino acid residues 4-7, 14-17, 54-57, 123-126, 264-267, 322-325, 375-378, 395-398, 559-562, 602-605, 618-621, 639-642, 703-706, 716-719, 745-748 and 764-767 of the IC23927 protein comprise casein kinase II phosphorylation sites. Accordingly, IC23927 proteins having at least one casein kinase II phosphorylation site are within the scope of the invention.

In another embodiment, an IC23927 molecule of the present invention is identified based on the presence of at least one tyrosine kinase phosphorylation site. As used herein, the term “tyrosine kinase phosphorylation site” includes an amino acid sequence of about 9 amino acid residues in length which includes a tyrosine residue which is potentially phosphorylated by a tyrosine kinase. Preferably, a tyrosine kinase phosphorylation site has the consensus sequence [RK]-x(2)-[DE]-x(3)-Y or [RK]-x(3)-[DE]-x(2)-Y [Y is the phosphorylation site]. Tyrosine kinase phosphorylation sites are described in, for example, Prosite PDOC00007 (http://www.expasy.ch/cgi-bin/nicedoc.p1?PDOC00007), the contents of which are incorporated herein by reference. Amino acid residues 617-625 of the IC23927 protein comprise tyrosine kinase phosphorylation sites. Accordingly, IC23927 proteins having at least one tyrosine kinase phosphorylation site are within the scope of the invention.

In another embodiment, an IC23927 molecule of the present invention is identified based on the presence of at least one N-myristoylation site. As used herein, the term “N-myristoylation site” includes an amino acid sequence of about 6 amino acid residues in length which includes an asparagine residue which is potentially acylated by the covalent addition of myristate. Preferably, a N-myristoylation site has the consensus sequence G-{EDRKHPFYW}-x(2)-[STAGCN]-{P} [G is the N-myristoylation site]. The N-terminal residue must be glycine. In position 2, uncharged residues are allowed. Charged residues, proline and large hydrophobic residues are not allowed. In positions 3 and 4, most, if not all, residues are allowed. In position 5, small uncharged residues are allowed (Ala, Ser, Thr, Cys, Asn and Gly). Serine is favored. In position 6, proline is not allowed. N-myristoylation sites are described in, for example, Prosite PDOC00008 (http://www.expasy.ch/cgi-bin/nicedoc.p1?PDOC00008), the contents of which are incorporated herein by reference. Amino acid residues 39-44, 217-222 and 468-473 of the IC23927 protein comprise N-myristoylation sites. Accordingly, IC23927 proteins having at least one N-myristoylation site are within the scope of the invention.

In another embodiment, an IC23927 molecule of the present invention is identified based on the presence of at least one amidation site. As used herein, the term “amidation site” includes an amino acid sequence of about 4 amino acid residues in length which includes a C-terminal residue which is potentially amidated. Preferably, an amidation site has the consensus sequence x-G-[RK]-[RK] [x is the amidation site]. Amidation sites are described in, for example, Prosite PDOC00009 (http://www.expasy.ch/cgi-bin/nicedoc.p1?PDOC00009), the contents of which are incorporated herein by reference. Amino acid residues 758-761 of the IC23927 protein comprise an amidation site. Accordingly, IC23927 proteins having at least one amidation site are within the scope of the invention.

In another embodiment, an IC23927 molecule of the present invention is identified based on the presence of an “ion transport protein domain” in the protein or corresponding nucleic acid molecule. As used herein, the term “ion transport protein domain” includes a protein domain having an amino acid sequence of about 200-300 amino acid residues and having a bit score for the alignment of the sequence to the ion transport protein domain Hidden Markov Model having Accession No. PF00520 of at about 25-100. Preferably, an ion transport protein domain includes at least about 225-275, or more preferably about 250 amino acid residues, and has a bit score for the alignment of the sequence to the ion transport protein domain Hidden Markov Model having Accession No. PF00520 of at least 25, 27, 30, 32, 35, 37, 38, 40, 42, 45, 50, 60, 70, 80, 90 or higher. To identify the presence of an ion transport protein domain in an IC23927 protein, and make the determination that a protein of interest has a particular profile, the amino acid sequence of the protein is searched against a database of known protein domains (e.g., the PFam database) using the default parameters (available at http://www.sanger.ac.uk/Software/Pfam/search.shtml). A search was performed against the PFam database resulting in the identification of an ion transport protein domain in the amino acid sequence of human IC23927 (SEQ ID NO:22) at about residues 437-686 of SEQ ID NO:22.

Isolated proteins of the present invention, preferably IC23927 proteins, have an amino acid sequence sufficiently identical to the amino acid sequence of SEQ ID NO:22 or are encoded by a nucleotide sequence sufficiently identical to SEQ ID NO:21 or 23. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, an “IC23927 activity”, “biological activity of IC23927” or “functional activity of IC23927”, refers to an activity exerted by an IC23927 protein, polypeptide or nucleic acid molecule in an IC23927 expressing cell or tissue in vivo or ex vivo, or in an reaction containing IC23927 protein, as determined in vitro, according to standard techniques. In one embodiment, an IC23927 activity is a direct activity, such a binding of an IC23927 target molecule to an IC23927 protein of the present invention or the transport of an IC23927 target molecule (e.g., across a cell membrane). As used herein, a “target molecule” is a molecule with which an IC23927 protein interacts in nature, such that IC23927-mediated function is achieved. An IC23927 target molecule can be a non-IC23927 molecule. In an exemplary embodiment, an IC23927 target molecule is an IC23927 ligand, e.g., an ionic ligand, for example, calcium, potassium or sodium. In a preferred embodiment, an IC23927 target molecule is calcium. In yet another embodiment, an IC23927 target molecule is GTP. Alternatively, an IC23927 activity is an indirect activity, such as a cellular signaling activity mediated by interaction of the IC23927 protein with an IC23927 ligand. The biological activities of IC23927 are described herein. For example, the IC23927 proteins of the present invention can have one or more of the following activities: (1) modulation of membrane excitability, (2) regulation of intracellular ion concentration, (3) modulation of membrane polarization (e.g., membrane polarization and/or depolarization), (4) regulation of action potential, (5) regulation of cellular signal transduction (e.g., regulation of neuronal signal transduction), (6) regulation of neurotransmitter release (e.g., from neuronal cells), (7) modulation of synaptic transmission, (8) regulation of neuronal excitability and/or plasticity, (9) regulation of muscle contraction, (10) regulation of cell activation (e.g., T cell activation), (11) regulation of cellular proliferation, growth, migration and/or differentiation, and/or (12) modulation of pain and/or pain signaling.

Accordingly, another embodiment of the invention features isolated IC23927 proteins and polypeptides having an IC23927 activity. Preferred proteins are IC23927 proteins having at least one transmembrane domain, and, preferably, an IC23927 activity. Other preferred proteins are IC23927 proteins having at least one ion transport protein domain and, preferably, an IC23927 activity. Yet other preferred proteins are IC23927 proteins having at least one site selected from the group consisting of an N-glycosylation site, a protein kinase C phosphorylation site, a casein kinase II phosphorylation site, a tyrosine kinase phosphorylation site, an N-myristoylation site and an amidation site and, preferably, an IC23927 activity. Yet other preferred proteins are IC23927 proteins having at least one transmembrane domain and/or an ion transport protein domain, at least at least one site selected from the group consisting of an N-glycosylation site, a protein kinase C phosphorylation site, a casein kinase II phosphorylation site, a tyrosine kinase phosphorylation site, an N-myristoylation site and an amidation site and, preferably, an IC23927 activity.

Additional preferred proteins have at least one transmembrane domain and/or an ion transport protein domain, at least at least one site selected from the group consisting of an N-glycosylation site, a protein kinase C phosphorylation site, a casein kinase II phosphorylation site, a tyrosine kinase phosphorylation site, an N-myristoylation site and an amidation site, and are, preferably, encoded by a nucleic acid molecule having a nucleotide sequence which hybridizes under stringent hybridization conditions to a nucleic acid molecule comprising the nucleotide sequence of SEQ ID NO:21 or 23.

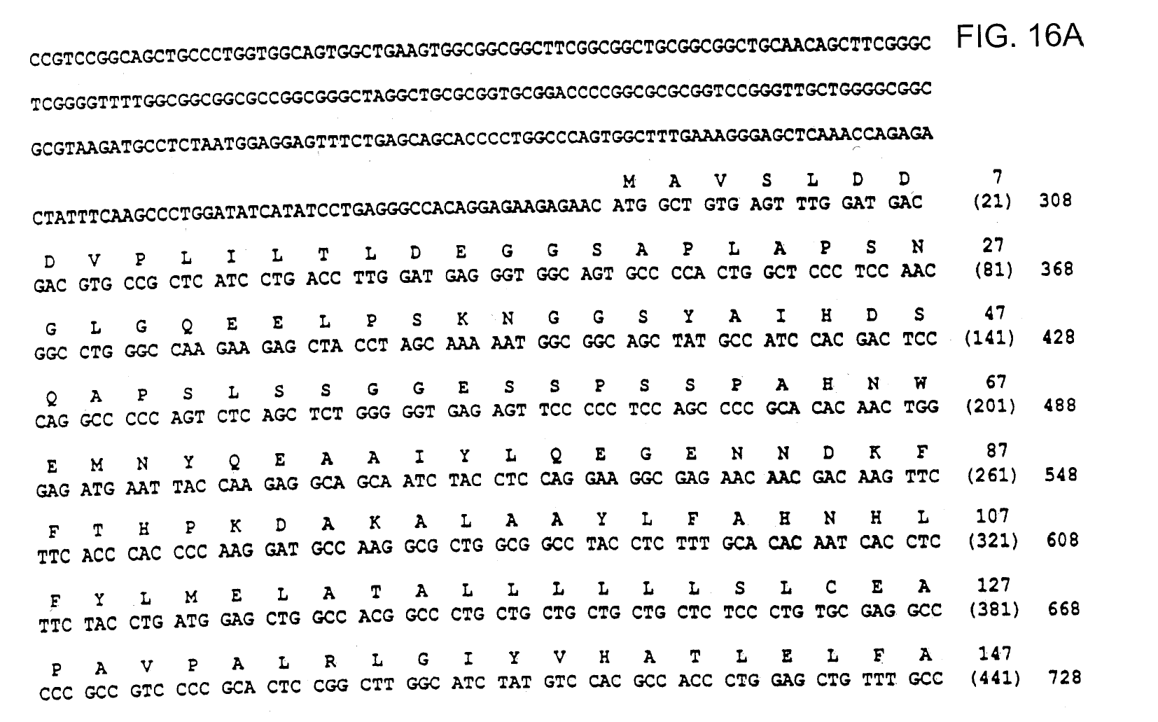

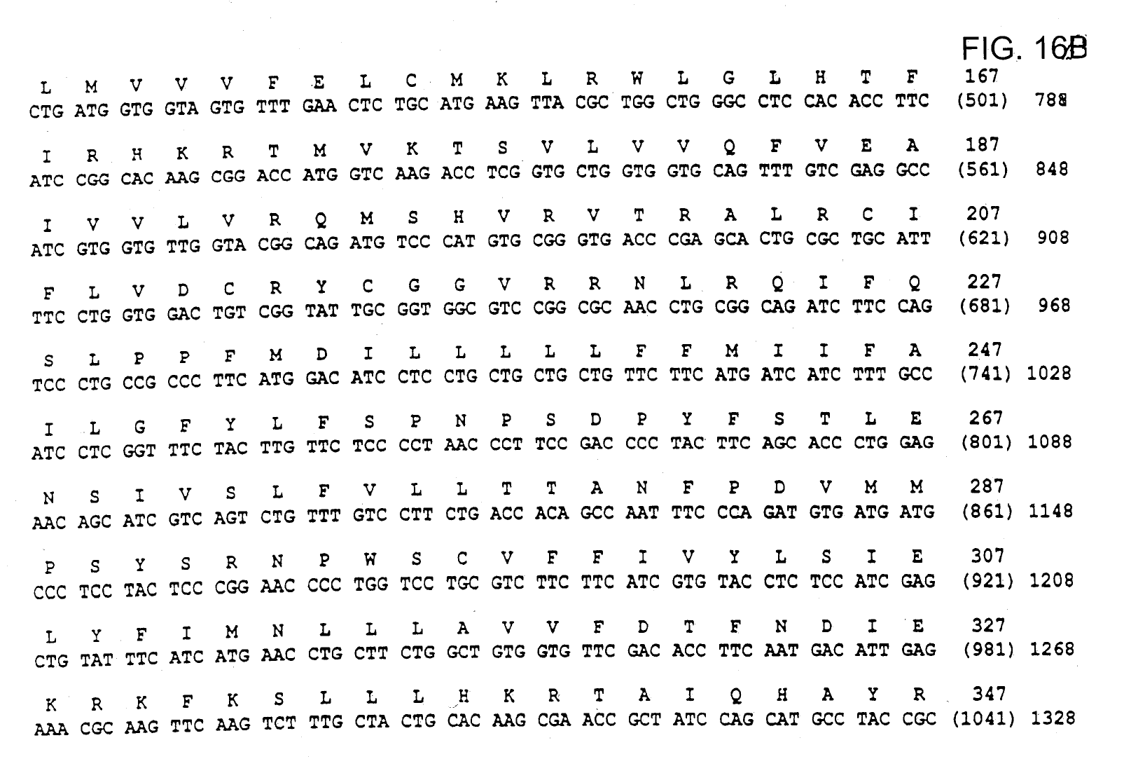

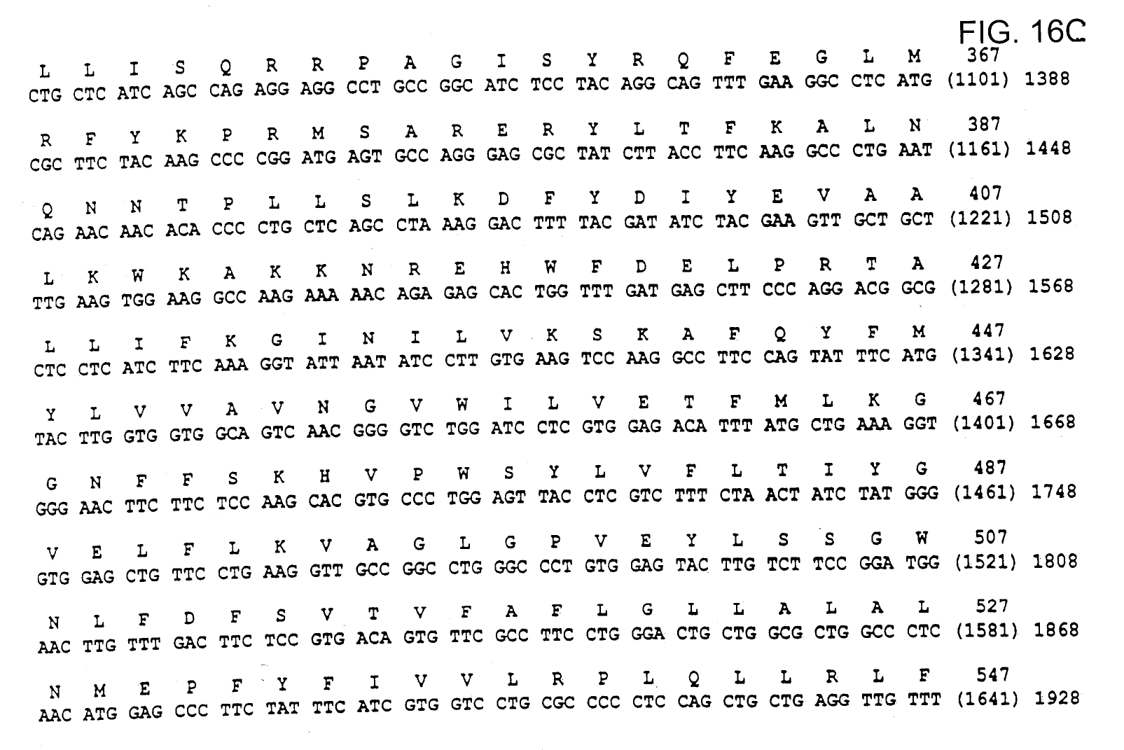

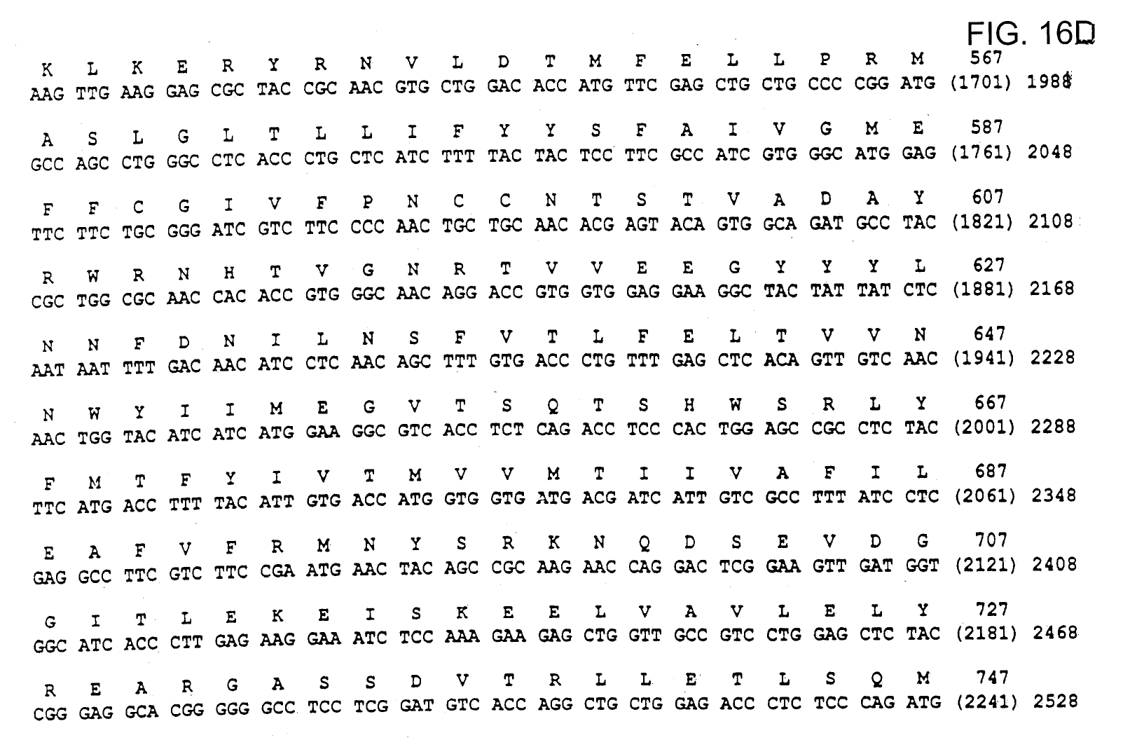

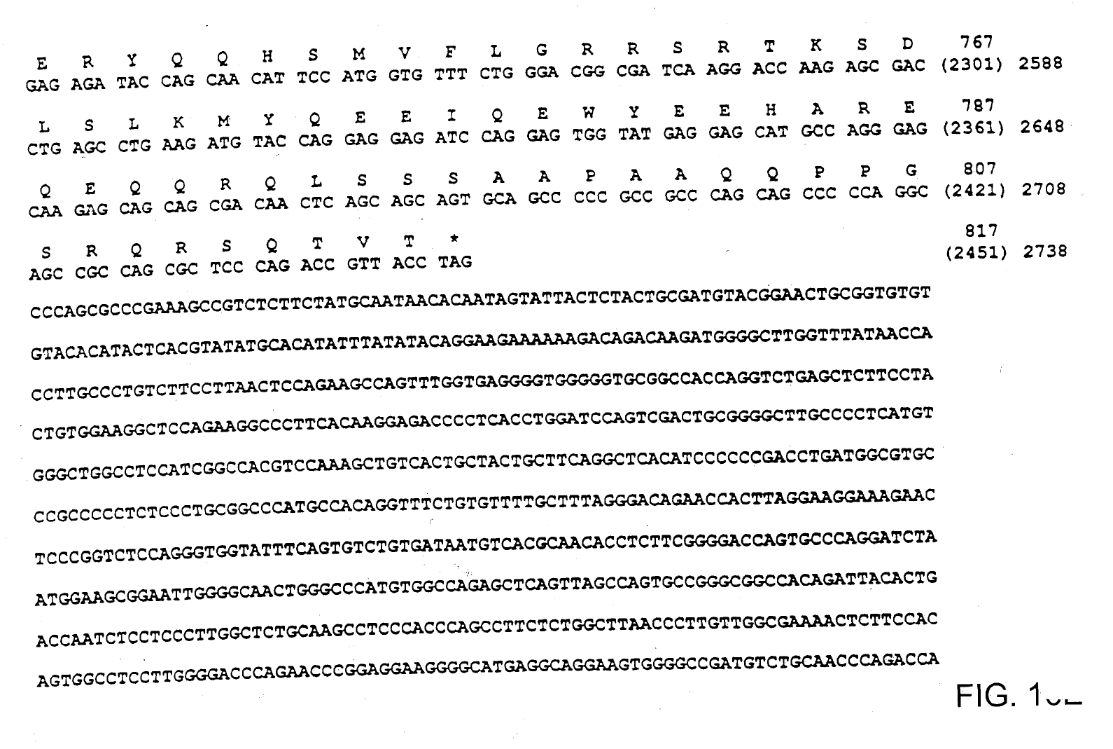

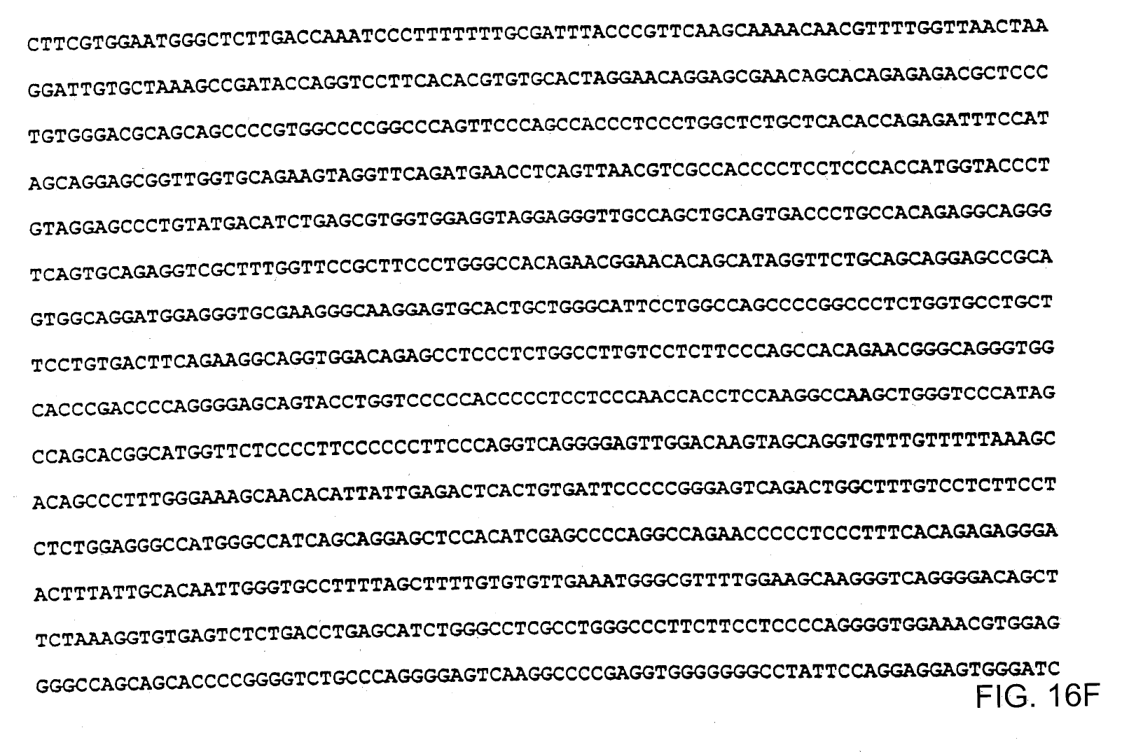

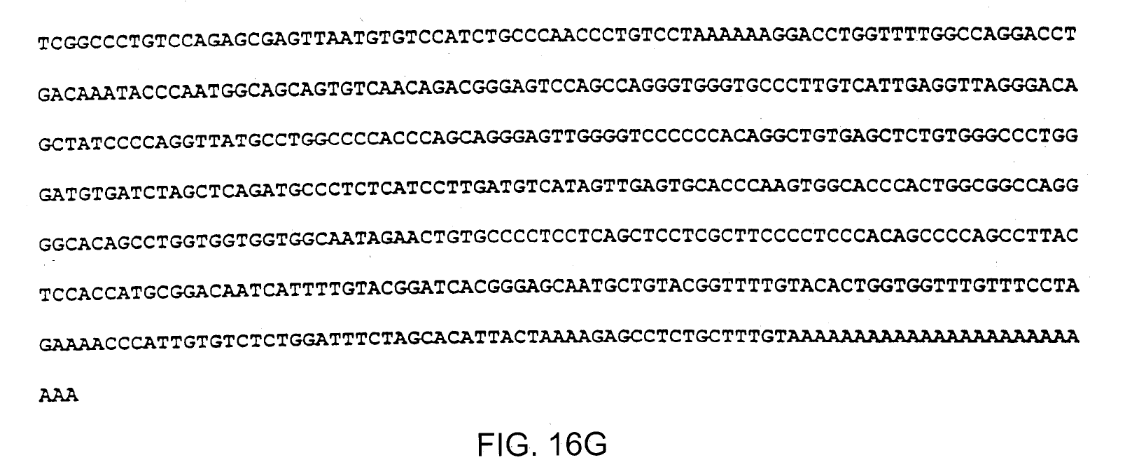

The nucleotide sequence of the isolated human IC23927 cDNA and the predicted amino acid sequence of the human IC23927 polypeptide are shown in FIGS. 16A-G and in SEQ ID NOs:21 and 22, respectively. A plasmid containing the nucleotide sequence encoding human IC23927 was deposited with the American Type Culture Collection (ATCC), 10801 University Boulevard, Manassas, Va. 20110-2209, USA, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The human IC23927 gene, which is approximately 5269 nucleotides in length, encodes a protein having a molecular weight of approximately 94 kD and which is approximately 816 amino acid residues in length.

Various aspects of the invention are described in further detail in the following subsections:

I. Isolated Nucleic Acid Molecules

One aspect of the invention pertains to isolated nucleic acid molecules that encode IC23927 proteins or biologically active portions thereof, as well as nucleic acid fragments sufficient for use as hybridization probes to identify IC23927-encoding nucleic acid molecules (e.g., IC23927 mRNA) and fragments for use as PCR primers for the amplification or mutation of IC23927 nucleic acid molecules. As used herein, the term “nucleic acid molecule” is intended to include DNA molecules (e.g., cDNA or genomic DNA) and RNA molecules (e.g, mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated IC23927 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:21 or 23, or the nucleotide sequence of the DNA insert of the plasmid deposited with ATCC as Accession Number ______, or a portion thereof, can be isolated using standard molecular biology techniques and the sequence information provided herein. Using all or a portion of the nucleic acid sequence of SEQ ID NO:21 or 23, or the nucleotide sequence of the DNA insert of the plasmid deposited with ATCC as Accession Number ______, as a hybridization probe, IC23927 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:21 or 23, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:21 or 23, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to IC23927 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:21. The sequence of SEQ ID NO:21 corresponds to the human IC23927 cDNA. This cDNA comprises sequences encoding the human IC23927 protein (i.e., “the coding region”, from nucleotides 288-2738), as well as 5′ untranslated sequences (nucleotides 1-287) and 3′ untranslated sequences (nucleotides 2399-5269). Alternatively, the nucleic acid molecule can comprise only the coding region of SEQ ID NO:21 (e.g., nucleotides 288-2738, corresponding to SEQ ID NO:23).

In another preferred embodiment, an isolated nucleic acid molecule of the invention comprises a nucleic acid molecule which is a complement of the nucleotide sequence shown in SEQ ID NO:21 or 23, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:21 or 23, or the nucleotide sequence of the DNA insert of the plasmid deposited with ATCC as Accession Number ______, is one which is sufficiently complementary to the nucleotide sequence shown in SEQ ID NO:21 or 23, or the nucleotide sequence of the DNA insert of the plasmid deposited with ATCC as Accession Number ______, such that it can hybridize to the nucleotide sequence shown in SEQ ID NO:21 or 23, or the nucleotide sequence of the DNA insert of the plasmid deposited with ATCC as Accession Number ______, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 96%, 97%, 98%, 99% or more identical to the entire length of the nucleotide sequence shown in SEQ ID NO:21 or 23, or the entire length of the nucleotide sequence of the DNA insert of the plasmid deposited with ATCC as Accession Number ______, or a portion of any of these nucleotide sequences.

Moreover, the nucleic acid molecule of the invention can comprise only a portion of the nucleic acid sequence of SEQ ID NO:21 or 23, or the nucleotide sequence of the DNA insert of the plasmid deposited with ATCC as Accession Number ______, for example, a fragment which can be used as a probe or primer or a fragment encoding a portion of an IC23927 protein, e.g., a biologically active portion of an IC23927 protein. The nucleotide sequence determined from the cloning of the IC23927 gene allows for the generation of probes and primers designed for use in identifying and/or cloning other IC23927 family members, as well as IC23927 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95, or 100 or more consecutive nucleotides of a sense sequence of SEQ ID NO:21 or 23, or the nucleotide sequence of the DNA insert of the plasmid deposited with ATCC as Accession Number ______, of an anti-sense sequence of SEQ ID NO:21 or 23, or the nucleotide sequence of the DNA insert of the plasmid deposited with ATCC as Accession Number ______, or of a naturally occurring allelic variant or mutant of SEQ ID NO:21 or 23, or the nucleotide sequence of the DNA insert of the plasmid deposited with ATCC as Accession Number ______. In one embodiment, a nucleic acid molecule of the present invention comprises a nucleotide sequence which is at least about (and in some embodiment, less than or greater than) 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2200-2300, 2300-2400, 2400-2500 or more nucleotides in length and hybridizes under stringent hybridization conditions to a nucleic acid molecule of SEQ ID NO:21 or 23, or the nucleotide sequence of the DNA insert of the plasmid deposited with ATCC as Accession Number ______.

Probes based on the IC23927 nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an IC23927 protein, such as by measuring a level of an IC23927-encoding nucleic acid in a sample of cells from a subject e.g., detecting IC23927 mRNA levels or determining whether a genomic IC23927 gene has been mutated or deleted.

A nucleic acid fragment encoding a “biologically active portion of an IC23927 protein” can be prepared by isolating a portion of the nucleotide sequence of SEQ ID NO:21 or 23, or the nucleotide sequence of the DNA insert of the plasmid deposited with ATCC as Accession Number ______, which encodes a polypeptide having an IC23927 biological activity (the biological activities of the IC23927 proteins are described herein), expressing the encoded portion of the IC23927 protein (e.g., by recombinant expression in vitro) and assessing the activity of the encoded portion of the IC23927 protein.

The invention further encompasses nucleic acid molecules that differ from the nucleotide sequence shown in SEQ ID NO:21 or 23, or the nucleotide sequence of the DNA insert of the plasmid deposited with ATCC as Accession Number ______, due to degeneracy of the genetic code and thus encode the same IC23927 proteins as those encoded by the nucleotide sequence shown in SEQ ID NO:21 or 23, or the nucleotide sequence of the DNA insert of the plasmid deposited with ATCC as Accession Number ______. In another embodiment, an isolated nucleic acid molecule of the invention has a nucleotide sequence encoding a protein having an amino acid sequence shown in SEQ ID NO:22.

In addition to the IC23927 nucleotide sequences shown in SEQ ID NO:21 or 23, or the nucleotide sequence of the DNA insert of the plasmid deposited with ATCC as Accession Number ______, it will be appreciated by those skilled in the art that DNA sequence polymorphisms that lead to changes in the amino acid sequences of the IC23927 proteins may exist within a population (e.g., the human population). Such genetic polymorphism in the IC23927 genes may exist among individuals within a population due to natural allelic variation. As used herein, the terms “gene” and “recombinant gene” refer to nucleic acid molecules which include an open reading frame encoding an IC23927 protein, preferably a mammalian IC23927 protein, and can further include non-coding regulatory sequences, and introns.

Allelic variants of human IC23927 include both functional and non-functional IC23927 proteins. Functional allelic variants are naturally occurring amino acid sequence variants of the human IC23927 protein that maintain the ability to bind an IC23927 ligand or transport such ligand and/or modulate membrane excitability or signal transduction. Functional allelic variants will typically contain only conservative substitution of one or more amino acids of SEQ ID NO:22, or substitution, deletion or insertion of non-critical residues in non-critical regions of the protein.

Non-functional allelic variants are naturally occurring amino acid sequence variants of the human IC23927 protein that do not have the ability to form functional calcium channels or to modulate membrane excitability. Non-functional allelic variants will typically contain a non-conservative substitution, a deletion, or insertion or premature truncation of the amino acid sequence of SEQ ID NO:22, or a substitution, insertion or deletion in critical residues or critical regions.

The present invention further provides non-human orthologues of the human IC23927 proteins. Orthologues of the human IC23927 protein are proteins that are isolated from non-non-human organisms and possess the same IC23927 ligand binding and/or modulation of membrane excitation mechanisms of the human IC23927 protein. Orthologues of the human IC23927 protein can readily be identified as comprising an amino acid sequence that is substantially identical to SEQ ID NO:22.

Moreover, nucleic acid molecules encoding other IC23927 family members and, thus, which have a nucleotide sequence which differs from the IC23927 sequences of SEQ ID NO:21 or 23, or the nucleotide sequence of the DNA insert of the plasmid deposited with ATCC as Accession Number ______ are intended to be within the scope of the invention. For example, another IC23927 cDNA can be identified based on the nucleotide sequence of human IC23927. Moreover, nucleic acid molecules encoding IC23927 proteins from different species, and which, thus, have a nucleotide sequence which differs from the IC23927 sequences of SEQ ID NO:21 or 23, or the nucleotide sequence of the DNA insert of the plasmid deposited with ATCC as Accession Number ______ are intended to be within the scope of the invention. For example, a mouse IC23927 cDNA can be identified based on the nucleotide sequence of a human IC23927.

Nucleic acid molecules corresponding to natural allelic variants and homologues of the IC23927 cDNAs of the invention can be isolated based on their homology to the IC23927 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the IC23927 cDNAs of the invention can further be isolated by mapping to the same chromosome or locus as the IC23927 gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:21 or 23, or the nucleotide sequence of the DNA insert of the plasmid deposited with ATCC as Accession Number ______. In other embodiment, the nucleic acid is at least 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2200-2300, 2300-2400, 2400-2500, or more nucleotides in length. As used herein, the term “hybridizes under stringent conditions” is intended to describe conditions for hybridization and washing under which nucleotide sequences at least 60% identical to each other typically remain hybridized to each other. Preferably, the conditions are such that sequences at least about 70%, more preferably at least about 80%, even more preferably at least about 85% or 90% identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50° C., preferably at 55° C., more preferably at 60° C., and even more preferably at 65° C. Preferably, an isolated nucleic acid molecule of the invention that hybridizes under stringent conditions to the sequence of SEQ ID NO:21 or 23 and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the IC23927 sequences that may exist in the population, the skilled artisan will further appreciate that changes can be introduced by mutation into the nucleotide sequences of SEQ ID NO:21 or 23, or the nucleotide sequence of the DNA insert of the plasmid deposited with ATCC as Accession Number ______, thereby leading to changes in the amino acid sequence of the encoded IC23927 proteins, without altering the functional ability of the IC23927 proteins. For example, nucleotide substitutions leading to amino acid substitutions at “non-essential” amino acid residues can be made in the sequence of SEQ ID NO:21 or 23, or the nucleotide sequence of the DNA insert of the plasmid deposited with ATCC as Accession Number ______. A “non-essential” amino acid residue is a residue that can be altered from the wild-type sequence of IC23927 (e.g., the sequence of SEQ ID NO:22) without altering the biological activity, whereas an “essential” amino acid residue is required for biological activity. For example, amino acid residues that are conserved among the IC23927 proteins of the present invention, e.g., those present in a transmembrane domain, are predicted to be particularly unamenable to alteration. Furthermore, additional amino acid residues that are conserved between the IC23927 proteins of the present invention and other members of the IC23927 family are not likely to be amenable to alteration.

Accordingly, another aspect of the invention pertains to nucleic acid molecules encoding IC23927 proteins that contain changes in amino acid residues that are not essential for activity. Such IC23927 proteins differ in amino acid sequence from SEQ ID NO:22, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 95%, 96%, 97%, 98%, 99% or more identical to SEQ ID NO:22.

An isolated nucleic acid molecule encoding an IC23927 protein identical to the protein of SEQ ID NO:22, can be created by introducing one or more nucleotide substitutions, additions or deletions into the nucleotide sequence of SEQ ID NO:21 or 23, or the nucleotide sequence of the DNA insert of the plasmid deposited with ATCC as Accession Number ______, such that one or more amino acid substitutions, additions or deletions are introduced into the encoded protein. Mutations can be introduced into SEQ ID NO:21 or 23, or the nucleotide sequence of the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g, aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an IC23927 protein is preferably replaced with another amino acid residue from the same side chain family. Alternatively, in another embodiment, mutations can be introduced randomly along all or part of an IC23927 coding sequence, such as by saturation mutagenesis, and the resultant mutants can be screened for IC23927 biological activity to identify mutants that retain activity. Following mutagenesis of SEQ ID NO:21 or 23, or the nucleotide sequence of the DNA insert of the plasmid deposited with ATCC as Accession Number ______, the encoded protein can be expressed recombinantly and the activity of the protein can be determined.

In a preferred embodiment, a mutant IC23927 protein can be assayed for the ability to (1) modulate membrane excitability, (2) regulate intracellular ion concentration, (3) modulate membrane polarization (e.g., membrane polarization and/or depolarization), (4) regulate action potential, (5) regulate cellular signal transduction, (6) regulate neurotransmitter release (e.g., from neuronal cells), (7) modulate synaptic transmission, (8) regulate neuronal excitability and/or plasticity, (9) regulate muscle contraction, (10) regulate cell activation (e.g., T cell activation) and (11) regulate cellular proliferation, growth, migration and/or differentiation.

In addition to the nucleic acid molecules encoding IC23927 proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire IC23927 coding strand, or to only a portion thereof. In one embodiment, an antisense nucleic acid molecule is antisense to a “coding region” of the coding strand of a nucleotide sequence encoding IC23927. The term “coding region” refers to the region of the nucleotide sequence comprising codons which are translated into amino acid residues (e.g., the coding region of human IC23927 corresponds to SEQ ID NO:23). In another embodiment, the antisense nucleic acid molecule is antisense to a “noncoding region” of the coding strand of a nucleotide sequence encoding IC23927. The term “noncoding region” refers to 5′ and 3′ sequences which flank the coding region that are not translated into amino acids (i.e., also referred to as 5′ and 3′ untranslated regions).

Given the coding strand sequences encoding IC23927 disclosed herein (e.g., SEQ ID NO:23), antisense nucleic acids of the invention can be designed according to the rules of Watson and Crick base pairing. The antisense nucleic acid molecule can be complementary to the entire coding region of IC23927 mRNA, but more preferably is an oligonucleotide which is antisense to only a portion of the coding or noncoding region of IC23927 mRNA. For example, the antisense oligonucleotide can be complementary to the region surrounding the translation start site of IC23927 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an IC23927 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave IC23927 mRNA transcripts to thereby inhibit translation of IC23927 mRNA. A ribozyme having specificity for an IC23927-encoding nucleic acid can be designed based upon the nucleotide sequence of an IC23927 cDNA disclosed herein (i.e., SEQ ID NO:21 or 23, or the nucleotide sequence of the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in an IC23927-encoding mRNA. See, e.g., Cech et al. U.S. Pat. No. 4,987,071; and Cech et al. U.S. Pat. No. 5,116,742. Alternatively, IC23927 mRNA can be used to select a catalytic RNA having a specific ribonuclease activity from a pool of RNA molecules. See, e.g., Bartel, D. and Szostak, J. W. (1993) Science 261:1411-1418.

Alternatively, IC23927 gene expression can be inhibited by targeting nucleotide sequences complementary to the regulatory region of the IC23927 (e.g., the IC23927 promoter and/or enhancers; e.g., nucleotides 1-287 of SEQ ID NO:21) to form triple helical structures that prevent transcription of the IC23927 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the IC23927 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g, DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of IC23927 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of IC23927 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of IC23927 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of IC23927 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous IC23927 gene within a cell line or microorganism may be modified by inserting a heterologous DNA regulatory element into the genome of a stable cell line or cloned microorganism such that the inserted regulatory element is operatively linked with the endogenous IC23927 gene. For example, an endogenous IC23927 gene which is normally “transcriptionally silent”, i.e., an IC23927 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous IC23927 gene may be activated by insertion of a promiscuous regulatory element that works across cell types.

A heterologous regulatory element may be inserted into a stable cell line or cloned microorganism, such that it is operatively linked with an endogenous IC23927 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated IC23927 Proteins and Anti-IC23927 Antibodies

One aspect of the invention pertains to isolated IC23927 proteins, and biologically active portions thereof, as well as polypeptide fragments suitable for use as immunogens to raise anti-IC23927 antibodies. In one embodiment, native IC23927 proteins can be isolated from cells or tissue sources by an appropriate purification scheme using standard protein purification techniques. In another embodiment, IC23927 proteins are produced by recombinant DNA techniques. Alternative to recombinant expression, an IC23927 protein or polypeptide can be synthesized chemically using standard peptide synthesis techniques.

An “isolated” or “purified” protein or biologically active portion thereof is substantially free of cellular material or other contaminating proteins from the cell or tissue source from which the IC23927 protein is derived, or substantially free from chemical precursors or other chemicals when chemically synthesized. The language “substantially free of cellular material” includes preparations of IC23927 protein in which the protein is separated from cellular components of the cells from which it is isolated or recombinantly produced. In one embodiment, the language “substantially free of cellular material” includes preparations of IC23927 protein having less than about 30% (by dry weight) of non-IC23927 protein (also referred to herein as a “contaminating protein”), more preferably less than about 20% of non-IC23927 protein, still more preferably less than about 10% of non-IC23927 protein, and most preferably less than about 5% non-IC23927 protein. When the IC23927 protein or biologically active portion thereof is recombinantly produced, it is also preferably substantially free of culture medium, i e., culture medium represents less than about 20%, more preferably less than about 10%, and most preferably less than about 5% of the volume of the protein preparation.

The language “substantially free of chemical precursors or other chemicals” includes preparations of IC23927 protein in which the protein is separated from chemical precursors or other chemicals which are involved in the synthesis of the protein. In one embodiment, the language “substantially free of chemical precursors or other chemicals” includes preparations of IC23927 protein having less than about 30% (by dry weight) of chemical precursors or non-IC23927 chemicals, more preferably less than about 20% chemical precursors or non-IC23927 chemicals, still more preferably less than about 10% chemical precursors or non-IC23927 chemicals, and most preferably less than about 5% chemical precursors or non-IC23927 chemicals.

As used herein, a “biologically active portion” of an IC23927 protein includes a fragment of an IC23927 protein which participates in an interaction between an IC23927 molecule and a non-IC23927 molecule. Biologically active portions of an IC23927 protein include peptides comprising amino acid sequences sufficiently identical to or derived from the amino acid sequence of the IC23927 protein, e.g., the amino acid sequence shown in SEQ ID NO:22, which include less amino acids than the full length IC23927 proteins, and exhibit at least one activity of an IC23927 protein. Typically, biologically active portions comprise a domain or motif with at least one activity of the IC23927 protein, e.g., modulating membrane excitation mechanisms. A biologically active portion of an IC23927 protein can be a polypeptide which is, for example, 25, 30, 35, 40, 45, 50, 75, 100, 125, 150, 175, 200, 225, 250, 275, 300, 316, 325, 350, 375, 400, 425, 450, 274, 500, 525, 550, 575, 600, 625, 650, 675, 700, 725, 750, 775, 800, or more amino acids in length. Biologically active portions of an IC23927 protein can be used as targets for developing agents which modulate an IC23927 mediated activity, e.g., a membrane excitation mechanism.

In one embodiment, a biologically active portion of an IC23927 protein comprises at least one transmembrane domain and/or an ion transport protein domain. It is to be understood that a preferred biologically active portion of an IC23927 protein of the present invention comprises at least one transmembrane domain and/or an ion transport protein and at least one site selected from the group consisting of an N-glycosylation site, a protein kinase C phosphorylation site, a casein kinase II phosphorylation site, a tyrosine kinase phosphorylation site, an N-myristoylation site and an amidation site. Moreover, other biologically active portions, in which other regions of the protein are deleted, can be prepared by recombinant techniques and evaluated for one or more of the functional activities of a native IC23927 protein.

In a preferred embodiment, the IC23927 protein has an amino acid sequence shown in SEQ ID NO:22. In other embodiments, the IC23927 protein is substantially identical to SEQ ID NO:22, and retains the functional activity of the protein of SEQ ID NO:22, yet differs in amino acid sequence due to natural allelic variation or mutagenesis, as described in detail in subsection I above. Accordingly, in another embodiment, the IC23927 protein is a protein which comprises an amino acid sequence at least about 50%, 55%, 60%, 65%, 70%, 75%, 80%, 85%, 90%, 95%, 96%, 97%, 98%, 99% or more identical to SEQ ID NO:22.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the IC23927 amino acid sequence of SEQ ID NO:22 having 816 amino acid residues, at least 245, 326, 407, 490, 571, 653 or 734 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 2 or 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to IC23927 nucleic acid molecules of the invention. BLAST protein searches can be performed with the XBLAST program, score=100, wordlength=3, and a Blosum62 matrix to obtain amino acid sequences homologous to IC23927 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides IC23927 chimeric or fusion proteins. As used herein, an IC23927 “chimeric protein” or “fusion protein” comprises an IC23927 polypeptide operatively linked to a non-IC23927 polypeptide. An “IC23927 polypeptide” refers to a polypeptide having an amino acid sequence corresponding to IC23927, whereas a “non-IC23927 polypeptide” refers to a polypeptide having an amino acid sequence corresponding to a protein which is not substantially homologous to the IC23927 protein, e.g., a protein which is different from the IC23927 protein and which is derived from the same or a different organism. Within an IC23927 fusion protein the IC23927 polypeptide can correspond to all or a portion of an IC23927 protein. In a preferred embodiment, an IC23927 fusion protein comprises at least one biologically active portion of an IC23927 protein. In another preferred embodiment, an IC23927 fusion protein comprises at least two biologically active portions of an IC23927 protein. Within the fusion protein, the term “operatively linked” is intended to indicate that the IC23927 polypeptide and the non-IC23927 polypeptide are fused in-frame to each other. The non-IC23927 polypeptide can be fused to the N-terminus or C-terminus of the IC23927 polypeptide.

For example, in one embodiment, the fusion protein is a GST-IC23927 fusion protein in which the IC23927 sequences are fused to the C-terminus of the GST sequences. Such fusion proteins can facilitate the purification of recombinant IC23927.

In another embodiment, the fusion protein is an IC23927 protein containing a heterologous signal sequence at its N-terminus. In certain host cells (e.g., bacterial or mammalian host cells), expression and/or secretion of IC23927 can be increased through use of a heterologous signal sequence.

The IC23927 fusion proteins of the invention can be incorporated into pharmaceutical compositions and administered to a subject in vivo. The IC23927 fusion proteins can be used to affect the bioavailability of an IC23927 ligand. Use of IC23927 fusion proteins may be useful therapeutically for the treatment of disorders caused by, for example, (i) aberrant modification or mutation of a gene encoding an IC23927 protein; (ii) mis-regulation of the IC23927 gene; and (iii) aberrant post-translational modification of an IC23927 protein.

Moreover, the IC23927-fusion proteins of the invention can be used as immunogens to produce anti-IC23927 antibodies in a subject, to purify IC23927 ligands and in screening assays to identify molecules which inhibit the interaction of IC23927 with an IC23927 ligand or target molecule.

Preferably, an IC23927 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An IC23927-encoding nucleic acid can be cloned into such an expression vector such that the fusion moiety is linked in-frame to the IC23927 protein.

The present invention also pertains to variants of the IC23927 proteins which function as either IC23927 agonists (mimetics) or as IC23927 antagonists. Variants of the IC23927 proteins can be generated by mutagenesis, e.g., discrete point mutation or truncation of an IC23927 protein. An agonist of the IC23927 proteins can retain substantially the same, or a subset, of the biological activities of the naturally occurring form of an IC23927 protein. An antagonist of an IC23927 protein can inhibit one or more of the activities of the naturally occurring form of the IC23927 protein by, for example, competitively modulating an IC23927-mediated activity of an IC23927 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the IC23927 protein.

In one embodiment, variants of an IC23927 protein which function as either IC23927 agonists (mimetics) or as IC23927 antagonists can be identified by screening combinatorial libraries of mutants, e.g., truncation mutants, of an IC23927 protein for IC23927 protein agonist or antagonist activity. In one embodiment, a variegated library of IC23927 variants is generated by combinatorial mutagenesis at the nucleic acid level and is encoded by a variegated gene library. A variegated library of IC23927 variants can be produced by, for example, enzymatically ligating a mixture of synthetic oligonucleotides into gene sequences such that a degenerate set of potential IC23927 sequences is expressible as individual polypeptides, or alternatively, as a set of larger fusion proteins (e.g., for phage display) containing the set of IC23927 sequences therein. There are a variety of methods which can be used to produce libraries of potential IC23927 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential IC23927 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of an IC23927 protein coding sequence can be used to generate a variegated population of IC23927 fragments for screening and subsequent selection of variants of an IC23927 protein. In one embodiment, a library of coding sequence fragments can be generated by treating a double stranded PCR fragment of an IC23927 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the IC23927 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of IC23927 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify IC23927 variants (Arkin and Youvan (1992) Proc. Natl. Acad. Sci. USA 89:7811-7815; Delagrave et al. (1993) Protein Engineering 6(3):327-331).

In one embodiment, cell based assays can be exploited to analyze a variegated IC23927 library. For example, a library of expression vectors can be transfected into a cell line, e.g., an endothelial cell line, which ordinarily responds to IC23927 in a particular IC23927 ligand-dependent manner. The transfected cells are then contacted with IC23927-ligand and the effect of expression of the mutant on signaling by IC23927 can be detected, e.g., by monitoring calcium, IP3, or diacylglycerol concentration, phosphorylation profile of intracellular proteins, or intracellular ion concentration. Plasmid DNA can then be recovered from the cells which score for inhibition, or alternatively, potentiation of signaling by the IC23927 ligand, and the individual clones further characterized.

An isolated IC23927 protein, or a portion or fragment thereof, can be used as an immunogen to generate antibodies that bind IC23927 using standard techniques for polyclonal and monoclonal antibody preparation. A full-length IC23927 protein can be used or, alternatively, the invention provides antigenic peptide fragments of IC23927 for use as immunogens. The antigenic peptide of IC23927 comprises at least 8 amino acid residues of the amino acid sequence shown in SEQ ID NO:22 and encompasses an epitope of IC23927 such that an antibody raised against the peptide forms a specific immune complex with IC23927. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of IC23927 that are located on the surface of the protein, e.g., hydrophilic regions, as well as regions with high antigenicity (see, for example, FIG. 17).

An IC23927 immunogen typically is used to prepare antibodies by immunizing a suitable subject, (e.g., rabbit, goat, mouse or other mammal) with the immunogen. An appropriate immunogenic preparation can contain, for example, recombinantly expressed IC23927 protein or a chemically synthesized IC23927 polypeptide. The preparation can further include an adjuvant, such as Freund's complete or incomplete adjuvant, or similar immunostimulatory agent. Immunization of a suitable subject with an immunogenic IC23927 preparation induces a polyclonal anti-IC23927 antibody response.

Accordingly, another aspect of the invention pertains to anti-IC23927 antibodies. The term “antibody” as used herein refers to immunoglobulin molecules and immunologically active portions of immunoglobulin molecules, i.e., molecules that contain an antigen binding site which specifically binds (immunoreacts with) an antigen, such as IC23927. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind IC23927. The term “monoclonal antibody” or “monoclonal antibody composition”, as used herein, refers to a population of antibody molecules that contain only one species of an antigen binding site capable of immunoreacting with a particular epitope of IC23927. A monoclonal antibody composition thus typically displays a single binding affinity for a particular IC23927 protein with which it immunoreacts.

Polyclonal anti-IC23927 antibodies can be prepared as described above by immunizing a suitable subject with an IC23927 immunogen. The anti-IC23927 antibody titer in the immunized subject can be monitored over time by standard techniques, such as with an enzyme linked immunosorbent assay (ELISA) using immobilized IC23927. If desired, the antibody molecules directed against IC23927 can be isolated from the mammal (e.g., from the blood) and further purified by well known techniques, such as protein A chromatography to obtain the IgG fraction. At an appropriate time after immunization, e.g., when the anti-IC23927 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med, 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an IC23927 immunogen as described above, and the culture supernatants of the resulting hybridoma cells are screened to identify a hybridoma producing a monoclonal antibody that binds IC23927.

Any of the many well known protocols used for fusing lymphocytes and immortalized cell lines can be applied for the purpose of generating an anti-IC23927 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind IC23927, e.g., using a standard ELISA assay.

Alternative to preparing monoclonal antibody-secreting hybridomas, a monoclonal anti-IC23927 antibody can be identified and isolated by screening a recombinant combinatorial immunoglobulin library (e.g., an antibody phage display library) with IC23927 to thereby isolate immunoglobulin library members that bind IC23927. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-IC23927 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-IC23927 antibody (e.g., monoclonal antibody) can be used to isolate IC23927 by standard techniques, such as affinity chromatography or immunoprecipitation. An anti-IC23927 antibody can facilitate the purification of natural IC23927 from cells and of recombinantly produced IC23927 expressed in host cells. Moreover, an anti-IC23927 antibody can be used to detect IC23927 protein (e.g., in a cellular lysate or cell supernatant) in order to evaluate the abundance and pattern of expression of the IC23927 protein. Anti-IC23927 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

II. Recombinant Expression Vectors and Host Cells

Another aspect of the invention pertains to vectors, preferably expression vectors, containing a nucleic acid encoding an IC23927 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., IC23927 proteins, mutant forms of IC23927 proteins, fusion proteins, and the like).

The recombinant expression vectors of the invention can be designed for expression of IC23927 proteins in prokaryotic or eukaryotic cells. For example, IC23927 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in IC23927 activity assays, (e.g., direct assays or competitive assays described in detail below), or to generate antibodies specific for IC23927 proteins, for example. In a preferred embodiment, an IC23927 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the IC23927 expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corporation, San Diego, Calif.).

Alternatively, IC23927 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to IC23927 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which an IC23927 nucleic acid molecule of the invention is introduced, e.g., an IC23927 nucleic acid molecule within a recombinant expression vector or an IC23927 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an IC23927 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an IC23927 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (ie., express) an IC23927 protein. Accordingly, the invention further provides methods for producing an IC23927 protein using the host cells of the invention. In one embodiment, the method comprises culturing the host cell of the invention (into which a recombinant expression vector encoding an IC23927 protein has been introduced) in a suitable medium such that an IC23927 protein is produced. In another embodiment, the method further comprises isolating an IC23927 protein from the medium or the host cell.

The host cells of the invention can also be used to produce non-human transgenic animals. For example, in one embodiment, a host cell of the invention is a fertilized oocyte or an embryonic stem cell into which IC23927-coding sequences have been introduced. Such host cells can then be used to create non-human transgenic animals in which exogenous IC23927 sequences have been introduced into their genome or homologous recombinant animals in which endogenous IC23927 sequences have been altered. Such animals are useful for studying the function and/or activity of an IC23927 and for identifying and/or evaluating modulators of IC23927 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous IC23927 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an IC23927-encoding nucleic acid into the male pronuclei of a fertilized oocyte, e.g., by microinjection, retroviral infection, and allowing the oocyte to develop in a pseudopregnant female foster animal. The IC23927 cDNA sequence of SEQ ID NO:21 can be introduced as a transgene into the genome of a non-human animal. Alternatively, a nonhuman homologue of a human IC23927 gene, such as a mouse or rat IC23927 gene, can be used as a transgene. Alternatively, an IC23927 gene homologue, such as another IC23927 family member, can be isolated based on hybridization to the IC23927 cDNA sequences of SEQ ID NO:21 or 23, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to an IC23927 transgene to direct expression of an IC23927 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of an IC23927 transgene in its genome and/or expression of IC23927 mRNA in tissues or cells of the animals. A transgenic founder animal can then be used to breed additional animals carrying the transgene. Moreover, transgenic animals carrying a transgene encoding an IC23927 protein can further be bred to other transgenic animals carrying other transgenes.

To create a homologous recombinant animal, a vector is prepared which contains at least a portion of an IC23927 gene into which a deletion, addition or substitution has been introduced to thereby alter, e.g., functionally disrupt, the IC23927 gene. The IC23927 gene can be a human gene (e.g., the cDNA of SEQ ID NO:23), but more preferably, is a non-human homologue of a human IC23927 gene (e.g., a cDNA isolated by stringent hybridization with the nucleotide sequence of SEQ ID NO:21). For example, a mouse IC23927 gene can be used to construct a homologous recombination nucleic acid molecule, e.g., a vector, suitable for altering an endogenous IC23927 gene in the mouse genome. In a preferred embodiment, the homologous recombination nucleic acid molecule is designed such that, upon homologous recombination, the endogenous IC23927 gene is functionally disrupted (i.e., no longer encodes a functional protein; also referred to as a “knock out” vector). Alternatively, the homologous recombination nucleic acid molecule can be designed such that, upon homologous recombination, the endogenous IC23927 gene is mutated or otherwise altered but still encodes functional protein (e.g., the upstream regulatory region can be altered to thereby alter the expression of the endogenous IC23927 protein). In the homologous recombination nucleic acid molecule, the altered portion of the IC23927 gene is flanked at its 5′ and 3′ ends by additional nucleic acid sequence of the IC23927 gene to allow for homologous recombination to occur between the exogenous IC23927 gene carried by the homologous recombination nucleic acid molecule and an endogenous IC23927 gene in a cell, e.g., an embryonic stem cell. The additional flanking IC23927 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced IC23927 gene has homologously recombined with the endogenous IC23927 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The IC23927 nucleic acid molecules, IC23927 proteins, fragments of IC23927 proteins, IC23927 modulators and anti-IC23927 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g, intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of an IC23927 protein or an anti-IC23927 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules, oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g, with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g.. Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, modulators and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, an IC23927 protein of the invention has one or more of the following activities: (1) modulation of membrane excitability, (2) regulation of intracellular ion concentration, (3) modulation of membrane polarization (e.g., membrane polarization and/or depolarization), (4) regulation of action potential, (5) regulation of cellular signal transduction, (6) regulation of neurotransmitter release (e.g., from neuronal cells), (7) modulation of synaptic transmission, (8) regulation of neuronal excitability and/or plasticity, (9) regulation of muscle contraction, (10) regulation of cell activation (e.g., T cell activation), (11) regulation of cellular proliferation, growth, migration and/or differentiation, and/or (12) modulation of pain and/or pain signaling.

The isolated nucleic acid molecules of the invention can be used, for example, to express IC23927 protein (e.g., via a recombinant expression vector in a host cell in gene therapy applications), to detect IC23927 mRNA (e.g., in a biological sample) or a genetic alteration in an IC23927 gene, and to modulate IC23927 activity, as described further below. The IC23927 proteins can be used to treat disorders characterized by insufficient or excessive production of an IC23927 ligand or production of IC23927 inhibitors. Moreover, modulation of IC23927 activity has particular application in treating pain and or pain disorders. Modulation of IC23927 activity includes, but is not limited to, increasing or enhancing the activity of IC23927 (e.g., increasing or enhancing IC23927 signaling), decreasing or inhibiting the activity of IC23927 (e.g., decreasing or inhibiting IC23927 signaling), regulating IC23927 cellular localization, trafficking and/or desensitization of 52871. In addition, the IC23927 proteins can be used to screen for naturally occurring IC23927 ligands, to screen for drugs or compounds which modulate IC23927 activity, as well as to treat disorders characterized by insufficient or excessive production of IC23927 protein or production of IC23927 protein forms which have decreased, aberrant or unwanted activity compared to IC23927 wild type protein (e.g., CNS disorders (such as neurodegenerative disorders), pain disorders, or disorders of cellular growth, differentiation, or migration. Moreover, the anti-IC23927 antibodies of the invention can be used to detect and isolate IC23927 proteins, to regulate the bioavailability of IC23927 proteins, and modulate IC23927 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to IC23927 proteins, have a stimulatory or inhibitory effect on, for example, IC23927 expression or IC23927 activity.

In one embodiment, the invention provides assays for screening candidate or test compounds which are ligands of an IC23927 protein or polypeptide or biologically active portion thereof. In another embodiment, the invention provides assays for screening candidate or test compounds which bind to or modulate the activity of an IC23927 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an IC23927 protein or biologically active portion thereof is contacted with a test compound and the ability of the test compound to modulate IC23927 activity is determined. Determining the ability of the test compound to modulate IC23927 activity can be accomplished by monitoring, for example, intracellular calcium, IP3, or diacylglycerol concentration, or phosphorylation profile of intracellular proteins. Preferably, Determining the ability of the test compound to modulate IC23927 activity can be accomplished by monitoring intracellular ligand (e.g., ion) concentration). The cell, for example, can be of mammalian origin, e.g., a neuronal cell, or a liver cell.

The ability of the test compound to modulate IC23927 binding to a ligand or target molecule can also be determined. Determining the ability of the test compound to modulate IC23927 binding to a ligand or target molecule can be accomplished, for example, by coupling the IC23927 ligand or target molecule with a radioisotope or enzymatic label such that binding of the IC23927 ligand or target molecule to IC23927 can be determined by detecting the labeled IC23927 ligand or target molecule in a complex. Alternatively, IC23927 could be coupled with a radioisotope or enzymatic label to monitor the ability of a test compound to modulate IC23927 binding to an IC23927 ligand or target molecule in a complex. Determining the ability of the test compound to bind IC23927 can be accomplished, for example, by coupling the compound with a radioisotope or enzymatic label such that binding of the compound to IC23927 can be determined by detecting the labeled IC23927 compound in a complex. For example, compounds (e.g., IC23927 target molecules) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., an IC23927 ligand or target molecule) to interact with IC23927 without the labeling of any of the interactants. For example, a microphysiometer can be used to detect the interaction of a compound with IC23927 without the labeling of either the compound or the IC23927. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and IC23927.

In another embodiment, an assay is a cell-based assay comprising contacting a cell expressing an IC23927 target molecule (e.g., an IC23927 ligand) with a test compound and determining the ability of the test compound to modulate (e.g., stimulate or inhibit) the activity of the IC23927 target molecule. Determining the ability of the test compound to modulate the activity of an IC23927 target molecule can be accomplished, for example, by determining the ability of the IC23927 protein to bind to or interact with the IC23927 target molecule or, alternatively, by determining the intracellular concentration of the target molecule or ligand.

Determining the ability of the IC23927 protein, or a biologically active fragment thereof, to bind to, interact with or transport an IC23927 target molecule (e.g., a ligand) can be accomplished by one of the methods described above for determining direct binding. In a preferred embodiment, determining the ability of the IC23927 protein to bind to, interact with or transport an IC23927 target molecule can be accomplished by determining the intracellular concentration or a secondary activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target using an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which an IC23927 protein or biologically active portion thereof is contacted with a test compound and the ability of the test compound to bind to the IC23927 protein or biologically active portion thereof is determined. Preferred biologically active portions of the IC23927 proteins to be used in assays of the present invention include fragments which participate in interactions with non-IC23927 molecules, e.g., fragments with high surface probability scores (see, for example, FIG. 17). Binding of the test compound to the IC23927 protein can be determined either directly or indirectly as described above. In a preferred embodiment, the assay includes contacting the IC23927 protein or biologically active portion thereof with a known compound which binds IC23927 to form an assay mixture, contacting the assay mixture with a test compound, and determining the ability of the test compound to interact with an IC23927 protein, wherein determining the ability of the test compound to interact with an IC23927 protein comprises determining the ability of the test compound to preferentially bind to IC23927 or biologically active portion thereof as compared to the known compound.

In another embodiment, the assay is a cell-free assay in which an IC23927 protein or biologically active portion thereof is contacted with a test compound and the ability of the test compound to modulate (e.g., stimulate or inhibit) the activity of the IC23927 protein or biologically active portion thereof is determined. Determining the ability of the test compound to modulate the activity of an IC23927 protein can be accomplished, for example, by determining the ability of the IC23927 protein to bind to an IC23927 target molecule by one of the methods described above for determining direct binding. Determining the ability of the IC23927 protein to bind to an IC23927 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an IC23927 protein can be accomplished by determining the ability of the IC23927 protein to further modulate the activity of a downstream effector of an IC23927 target molecule. For example, the activity of the effector molecule on an appropriate target can be determined or the binding of the effector to an appropriate target can be determined as previously described.

In yet another embodiment, the cell-free assay involves contacting an IC23927 protein or biologically active portion thereof with a known compound which binds the IC23927 protein to form an assay mixture, contacting the assay mixture with a test compound, and determining the ability of the test compound to interact with the IC23927 protein, wherein determining the ability of the test compound to interact with the IC23927 protein comprises determining the ability of the IC23927 protein to preferentially bind to or modulate the activity of an IC23927 target molecule.

In more than one embodiment of the above assay methods of the present invention, it may be desirable to immobilize either IC23927 or its target molecule to facilitate separation of complexed from uncomplexed forms of one or both of the proteins, as well as to accommodate automation of the assay. Binding of a test compound to an IC23927 protein, or interaction of an IC23927 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/IC23927 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or IC23927 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of IC23927 binding or activity determined using standard techniques.

Other techniques for immobilizing proteins on matrices can also be used in the screening assays of the invention. For example, either an IC23927 protein or an IC23927 target molecule can be immobilized utilizing conjugation of biotin and streptavidin. Biotinylated IC23927 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with IC23927 protein or target molecules but which do not interfere with binding of the IC23927 protein to its target molecule can be derivatized to the wells of the plate, and unbound target or IC23927 protein trapped in the wells by antibody conjugation. Methods for detecting such complexes, in addition to those described above for the GST-immobilized complexes, include immunodetection of complexes using antibodies reactive with the IC23927 protein or target molecule, as well as enzyme-linked assays which rely on detecting an enzymatic activity associated with the IC23927 protein or target molecule.

In another embodiment, modulators of IC23927 expression are identified in a method wherein a cell is contacted with a candidate compound and the expression of IC23927 mRNA or protein in the cell is determined. The level of expression of IC23927 mRNA or protein in the presence of the candidate compound is compared to the level of expression of IC23927 mRNA or protein in the absence of the candidate compound. The candidate compound can then be identified as a modulator of IC23927 expression based on this comparison. For example, when expression of IC23927 mRNA or protein is greater (statistically significantly greater) in the presence of the candidate compound than in its absence, the candidate compound is identified as a stimulator of IC23927 mRNA or protein expression. Alternatively, when expression of IC23927 mRNA or protein is less (statistically significantly less) in the presence of the candidate compound than in its absence, the candidate compound is identified as an inhibitor of IC23927 mRNA or protein expression. The level of IC23927 mRNA or protein expression in the cells can be determined by methods described herein for detecting IC23927 mRNA or protein.

In yet another aspect of the invention, the IC23927 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with IC23927 (“IC23927-binding proteins” or “IC23927-bp”) and are involved in IC23927 activity. Such IC23927-binding proteins are also likely to be involved in the propagation of signals by the IC23927 proteins or IC23927 targets as, for example, downstream elements of an IC23927-mediated signaling pathway. Alternatively, such IC23927-binding proteins are likely to be IC23927 inhibitors.

The two-hybrid system is based on the modular nature of most transcription factors, which consist of separable DNA-binding and activation domains. Briefly, the assay utilizes two different DNA constructs. In one construct, the gene that codes for an IC23927 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an IC23927-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the IC23927 protein.

In another aspect, the invention pertains to a combination of two or more of the assays described herein. For example, a modulating agent can be identified using a cell-based or a cell free assay, and the ability of the agent to modulate the activity of an IC23927 protein can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., an IC23927 modulating agent, an antisense IC23927 nucleic acid molecule, an IC23927-specific antibody, or an IC23927-binding partner) can be used in an animal model to determine the efficacy, toxicity, or side effects of treatment with such an agent. Alternatively, an agent identified as described herein can be used in an animal model to determine the mechanism of action of such an agent. Models for studying pain in vivo include rat models of neuropathic pain caused by methods such as intraperitoneal administration of Taxol (Authier et al. (2000) Brain Res. 887:239-249), chronic constriction injury (CCI), partial sciatic nerve transection (Linenlaub and Sommer (2000) Pain 89:97-106), transection of the tibial and sural nerves (Lee et al. (2000) Neurosci. Lett. 291:29-32), the spared nerve injury model (Decosterd and Woolf (2000) Pain 87:149-158), cuffing the sciatic nerve (Pitcher and Henry (2000) Eur. J. Neurosci. 12:2006-2020), unilateral tight ligation (Esser and Sawynok (2000) Eur. J. Pharmacol. 399:131-139), L5 spinal nerve ligation (Honroe et al. (2000) Neurosci. 98:585-598), and photochemically induced ischemic nerve injury (Hao et al. (2000) Exp. Neurol. 163:231 -238); rat models of nociceptive pain caused by methods such as the Chung Method, the Bennett Method, and intraperitoneal administration of complete Freund's adjuvant (CFA) (Abdi et al. (2000) Anesth. Analg. 91:955-959); rat models of post-incisional pain caused by incising the skin and fascia of a hind paw (Olivera and Prado (2000) Braz. J. Med. Biol. Res. 33:957-960); rat models of cancer pain caused by methods such as injecting osteolytic sarcoma cells into the femur (Honroe et al. (2000) Neurosci. 98:585-598); and rat models of visceral pain caused by methods such as intraperitoneal administration of cyclophosphamide.

Various methods of determining an animal's response to pain are known in the art. Examples of such methods include, but are not limited to brief intense exposure to a focused heat source, administration of a noxious chemical subcutaneously, the tail flick test, the hot plate test, the formalin test, Von Frey threshold, and testing for stress-induced analgesia (et al., by restraint, foot shock, and/or cold water swim) (Crawley (2000) What's Wrong With My Mouse? Wiley-Liss pp. 72-75).

Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the IC23927 nucleotide sequences, described herein, can be used to map the location of the IC23927 genes on a chromosome. The mapping of the IC23927 sequences to chromosomes is an important first step in correlating these sequences with genes associated with disease.

Briefly, IC23927 genes can be mapped to chromosomes by preparing PCR primers (preferably 15-25 bp in length) from the IC23927 nucleotide sequences. Computer analysis of the IC23927 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the IC23927 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the IC23927 nucleotide sequences to design oligonucleotide primers, sublocalization can be achieved with panels of fragments from specific chromosomes. Other mapping strategies which can similarly be used to map an IC23927 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the IC23927 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The IC23927 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the IC23927 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The IC23927 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:21 can comfortably provide positive individual identification with a panel of perhaps 10 to 1,000 primers which each yield a noncoding amplified sequence of 100 bases. If predicted coding sequences, such as those in SEQ ID NO:23 are used, a more appropriate number of primers for positive individual identification would be 500-2,000.

If a panel of reagents from IC23927 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of IC23927 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:21 are particularly appropriate for this use as greater numbers of polymorphisms occur in the noncoding regions, making it easier to differentiate individuals using this technique. Examples of polynucleotide reagents include the IC23927 nucleotide sequences or portions thereof, e.g., fragments derived from the noncoding regions of SEQ ID NO:21 having a length of at least 20 bases, preferably at least 30 bases.

The IC23927 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such IC23927 probes can be used to identify tissue by species and/or by organ type.

In a similar fashion, these reagents, e.g., IC23927 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining IC23927 protein and/or nucleic acid expression as well as IC23927 activity, in the context of a biological sample (e.g., blood, serum, cells, tissue) to thereby determine whether an individual is afflicted with a disease or disorder, or is at risk of developing a disorder, associated with aberrant or unwanted IC23927 expression or activity. The invention also provides for prognostic (or predictive) assays for determining whether an individual is at risk of developing a disorder associated with IC23927 protein, nucleic acid expression or activity. For example, mutations in an IC23927 gene can be assayed in a biological sample. Such assays can be used for prognostic or predictive purpose to thereby prophylactically treat an individual prior to the onset of a disorder characterized by or associated with IC23927 protein, nucleic acid expression or activity.

Another aspect of the invention pertains to monitoring the influence of agents (e.g., drugs, compounds) on the expression or activity of IC23927 in clinical trials.

These and other agents are described in further detail in the following sections.

1. Diagnostic Assays

An exemplary method for detecting the presence or absence of IC23927 protein or nucleic acid in a biological sample involves obtaining a biological sample from a test subject and contacting the biological sample with a compound or an agent capable of detecting IC23927 protein or nucleic acid (e.g., mRNA, or genomic DNA) that encodes IC23927 protein such that the presence of IC23927 protein or nucleic acid is detected in the biological sample. A preferred agent for detecting IC23927 mRNA or genomic DNA is a labeled nucleic acid probe capable of hybridizing to IC23927 mRNA or genomic DNA. The nucleic acid probe can be, for example, the IC23927 nucleic acid set forth in SEQ ID NO:21 or 23, or the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to IC23927 mRNA or genomic DNA. Other suitable probes for use in the diagnostic assays of the invention are described herein.

A preferred agent for detecting IC23927 protein is an antibody capable of binding to IC23927 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect IC23927 mRNA, protein, or genomic DNA in a biological sample in vitro as well as in vivo. For example, in vitro techniques for detection of IC23927 mRNA include Northern hybridizations and in situ hybridizations. In vitro techniques for detection of IC23927 protein include enzyme linked immunosorbent assays (ELISAs), Western blots, immunoprecipitations and immunofluorescence. In vitro techniques for detection of IC23927 genomic DNA include Southern hybridizations. Furthermore, in vivo techniques for detection of IC23927 protein include introducing into a subject a labeled anti-IC23927 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting IC23927 protein, mRNA, or genomic DNA, such that the presence of IC23927 protein, mRNA or genomic DNA is detected in the biological sample, and comparing the presence of IC23927 protein, mRNA or genomic DNA in the control sample with the presence of IC23927 protein, mRNA or genomic DNA in the test sample.

The invention also encompasses kits for detecting the presence of IC23927 in a biological sample. For example, the kit can comprise a labeled compound or agent capable of detecting IC23927 protein or mRNA in a biological sample; means for determining the amount of IC23927 in the sample; and means for comparing the amount of IC23927 in the sample with a standard. The compound or agent can be packaged in a suitable container. The kit can further comprise instructions for using the kit to detect IC23927 protein or nucleic acid.

2. Prognostic Assays

The diagnostic methods described herein can furthermore be utilized to identify subjects having or at risk of developing a disease or disorder associated with aberrant or unwanted IC23927 expression or activity. As used herein, the term “aberrant” includes an IC23927 expression or activity which deviates from the wild type IC23927 expression or activity. Aberrant expression or activity includes increased or decreased expression or activity, as well as expression or activity which does not follow the wild type developmental pattern of expression or the subcellular pattern of expression. For example, aberrant IC23927 expression or activity is intended to include the cases in which a mutation in the IC23927 gene causes the IC23927 gene to be under-expressed or over-expressed and situations in which such mutations result in a non-functional IC23927 protein or a protein which does not function in a wild-type fashion, e.g., a protein which does not interact with an IC23927 target, e.g., a non-ion channel subunit or ligand, or one which interacts with a non-IC23927 target molecule, e.g. a non-ion channel subunit or ligand. As used herein, the term “unwanted” includes an unwanted phenomenon involved in a biological response, such as cellular proliferation. For example, the term unwanted includes an IC23927 expression or activity which is undesirable in a subject.

The assays described herein, such as the preceding diagnostic assays or the following assays, can be utilized to identify a subject having or at risk of developing a disorder associated with a misregulation in IC23927 protein activity or nucleic acid expression, such as a CNS disorder (e.g., a neurodegenerative disorder, a pain disorder, or a cellular proliferation, growth, differentiation, or migration disorder). Alternatively, the prognostic assays can be utilized to identify a subject having or at risk for developing a disorder associated with a misregulation in IC23927 protein activity or nucleic acid expression, such as a CNS disorder, a pain disorder, or a cellular proliferation, growth, differentiation, or migration disorder. Thus, the present invention provides a method for identifying a disease or disorder associated with aberrant or unwanted IC23927 expression or activity in which a test sample is obtained from a subject and IC23927 protein or nucleic acid (e.g., mRNA or genomic DNA) is detected, wherein the presence of IC23927 protein or nucleic acid is diagnostic for a subject having or at risk of developing a disease or disorder associated with aberrant or unwanted IC23927 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted IC23927 expression or activity. For example, such methods can be used to determine whether a subject can be effectively treated with an agent for a CNS disorder, pain disorder, or a cellular proliferation, growth, differentiation, or migration disorder. Thus, the present invention provides methods for determining whether a subject can be effectively treated with an agent for a disorder associated with aberrant or unwanted IC23927 expression or activity in which a test sample is obtained and IC23927 protein or nucleic acid expression or activity is detected (e.g., wherein the abundance of IC23927 protein or nucleic acid expression or activity is diagnostic for a subject that can be administered the agent to treat a disorder associated with aberrant or unwanted IC23927 expression or activity).

The methods of the invention can also be used to detect genetic alterations in an IC23927 gene, thereby determining if a subject with the altered gene is at risk for a disorder characterized by misregulation in IC23927 protein activity or nucleic acid expression, such as a CNS disorder, pain disorder, or a disorder of cellular growth, differentiation, or migration. In preferred embodiments, the methods include detecting, in a sample of cells from the subject, the presence or absence of a genetic alteration characterized by at least one of an alteration affecting the integrity of a gene encoding an IC23927-protein, or the mis-expression of the IC23927 gene. For example, such genetic alterations can be detected by ascertaining the existence of at least one of 1) a deletion of one or more nucleotides from an IC23927 gene; 2) an addition of one or more nucleotides to an IC23927 gene; 3) a substitution of one or more nucleotides of an IC23927 gene, 4) a chromosomal rearrangement of an IC23927 gene; 5) an alteration in the level of a messenger RNA transcript of an IC23927 gene, 6) aberrant modification of an IC23927 gene, such as of the methylation pattern of the genomic DNA, 7) the presence of a non-wild type splicing pattern of a messenger RNA transcript of an IC23927 gene, 8) a non-wild type level of an IC23927-protein, 9) allelic loss of an IC23927 gene, and 10) inappropriate post-translational modification of an IC23927-protein. As described herein, there are a large number of assays known in the art which can be used for detecting alterations in an IC23927 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the IC23927-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to an IC23927 gene under conditions such that hybridization and amplification of the IC23927-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an IC23927 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in IC23927 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in IC23927 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the IC23927 gene and detect mutations by comparing the sequence of the sample IC23927 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the IC23927 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type IC23927 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in IC23927 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an IC23927 sequence, e.g., a wild-type IC23927 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in IC23927 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control IC23927 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an IC23927 gene.

Furthermore, any cell type or tissue in which IC23927 is expressed may be utilized in the prognostic assays described herein.

3. Monitoring of Effects During Clinical Trials

Monitoring the influence of agents (e.g., drugs) on the expression or activity of an IC23927 protein (e.g., the modulation of membrane excitability) can be applied not only in basic drug screening, but also in clinical trials. For example, the effectiveness of an agent determined by a screening assay as described herein to increase IC23927 gene expression, protein levels, or upregulate IC23927 activity, can be monitored in clinical trials of subjects exhibiting decreased IC23927 gene expression, protein levels, or downregulated IC23927 activity. Alternatively, the effectiveness of an agent determined by a screening assay to decrease IC23927 gene expression, protein levels, or downregulate IC23927 activity, can be monitored in clinical trials of subjects exhibiting increased IC23927 gene expression, protein levels, or upregulated IC23927 activity. In such clinical trials, the expression or activity of an IC23927 gene, and preferably, other genes that have been implicated in, for example, an IC23927-associated disorder can be used as a “read out” or markers of the phenotype of a particular cell.

For example, and not by way of limitation, genes, including IC23927, that are modulated in cells by treatment with an agent (e.g., compound, drug or small molecule) which modulates IC23927 activity (e.g., identified in a screening assay as described herein) can be identified. Thus, to study the effect of agents on IC23927-associated disorders (e.g., disorders characterized by deregulated signaling, e.g., pain disorders, or membrane excitation), for example, in a clinical trial, cells can be isolated and RNA prepared and analyzed for the levels of expression of IC23927 and other genes implicated in the IC23927-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of IC23927 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an IC23927 protein, mRNA, or genomic DNA in the preadministration sample; (iii) obtaining one or more post-administration samples from the subject; (iv) detecting the level of expression or activity of the IC23927 protein, mRNA, or genomic DNA in the post-administration samples; (v) comparing the level of expression or activity of the IC23927 protein, mRNA, or genomic DNA in the pre-administration sample with the IC23927 protein, mRNA, or genomic DNA in the post administration sample or samples; and (vi) altering the administration of the agent to the subject accordingly. For example, increased administration of the agent may be desirable to increase the expression or activity of IC23927 to higher levels than detected, i.e., to increase the effectiveness of the agent. Alternatively, decreased administration of the agent may be desirable to decrease expression or activity of IC23927 to lower levels than detected, i.e. to decrease the effectiveness of the agent. According to such an embodiment, IC23927 expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted IC23927 expression or activity, e.g. a CNS disorder, pain disorder, or a cellular proliferation, growth, differentiation, or migration disorder. “Treatment”, or “treating” as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. A therapeutic agent includes, but is not limited to, small molecules, peptides, antibodies, ribozymes and antisense oligonucleotides. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the IC23927 molecules of the present invention or IC23927 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted IC23927 expression or activity, by administering to the subject an IC23927 or an agent which modulates IC23927 expression or at least one IC23927 activity. Subjects at risk for a disease which is caused or contributed to by aberrant or unwanted IC23927 expression or activity can be identified by, for example, any or a combination of diagnostic or prognostic assays as described herein. Administration of a prophylactic agent can occur prior to the manifestation of symptoms characteristic of the IC23927 aberrancy, such that a disease or disorder is prevented or, alternatively, delayed in its progression. Depending on the type of IC23927 aberrancy, for example, an IC23927, IC23927 agonist or IC23927 antagonist agent can be used for treating the subject. The appropriate agent can be determined based on screening assays described herein.

2. Therapeutic Methods

Another aspect of the invention pertains to methods of modulating IC23927 expression or activity for therapeutic purposes. Accordingly, in an exemplary embodiment, the modulatory method of the invention involves contacting a cell with an IC23927 or agent that modulates one or more of the activities of IC23927 protein activity associated with the cell. An agent that modulates IC23927 protein activity can be an agent as described herein, such as a nucleic acid or a protein, a naturally-occurring target molecule of an IC23927 protein (e.g., an IC23927 ligand or binding partner), an IC23927 antibody, an IC23927 agonist or antagonist, a peptidomimetic of an IC23927 agonist or antagonist, or other small molecule. In one embodiment, the agent stimulates one or more IC23927 activities. Examples of such stimulatory agents include active IC23927 protein and a nucleic acid molecule encoding IC23927 that has been introduced into the cell. In another embodiment, the agent inhibits one or more IC23927 activities. Examples of such inhibitory agents include antisense IC23927 nucleic acid molecules, anti-IC23927 antibodies, and IC23927 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of an IC23927 protein or nucleic acid molecule. In one embodiment, the method involves administering an agent (e.g., an agent identified by a screening assay described herein), or combination of agents that modulates (e.g., upregulates or downregulates) IC23927 expression or activity. In another embodiment, the method involves administering an IC23927 protein or nucleic acid molecule as therapy to compensate for reduced, aberrant, or unwanted IC23927 expression or activity.

Stimulation of IC23927 activity is desirable in situations in which IC23927 is abnormally downregulated and/or in which increased IC23927 activity is likely to have a beneficial effect. Likewise, inhibition of IC23927 activity is desirable in situations in which IC23927 is abnormally upregulated and/or in which decreased IC23927 activity is likely to have a beneficial effect.

3. Pharmacogenomics

The IC23927 molecules of the present invention, as well as agents, or modulators which have a stimulatory or inhibitory effect on IC23927 activity (e.g., IC23927 gene expression) as identified by a screening assay described herein can be administered to individuals to treat (prophylactically or therapeutically) IC23927-associated disorders (e.g., proliferative disorders) associated with aberrant or unwanted IC23927 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an IC23927 molecule or IC23927 modulator as well as tailoring the dosage and/or therapeutic regimen of treatment with an IC23927 molecule or IC23927 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., an IC23927 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an IC23927 molecule or IC23927 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an IC23927 molecule or IC23927 modulator, such as a modulator identified by one of the exemplary screening assays described herein.

4. Use of IC23927 Molecules as Surrogate Markers

The IC23927 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the IC23927 molecules of the invention may be detected, and may be correlated with one or more biological states in vivo. For example, the IC23927 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The IC23927 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a IC23927 marker) transcription or expression, the amplified marker may be in a quantity which is more readily detectable than the drug itself. Also, the marker may be more easily detected due to the nature of the marker itself; for example, using the methods described herein, anti-IC23927 antibodies may be employed in an immune-based detection system for a IC23927 protein marker, or IC23927-specific radiolabeled probes may be used to detect a IC23927 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The IC23927 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., IC23927 protein or RNA) for specific tumor markers in a subject, a drug or course of treatment may be selected that is optimized for the treatment of the specific tumor likely to be present in the subject. Similarly, the presence or absence of a specific sequence mutation in IC23927 DNA may correlate IC23927 drug response. The use of pharmacogenomic markers therefore permits the application of the most appropriate treatment for each subject without having to administer the therapy.

VI. Electronic Apparatus Readable Media and Arrays

Electronic apparatus readable media comprising IC23927 sequence information is also provided. As used herein, “IC23927 sequence information” refers to any nucleotide and/or amino acid sequence information particular to the IC23927 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said IC23927 sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon IC23927 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the IC23927 sequence information.

A variety of software programs and formats can be used to store the sequence information on the electronic apparatus readable medium. For example, the sequence information can be represented in a word processing text file, formatted in commercially-available software such as WordPerfect and Microsoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon the IC23927 sequence information.

By providing IC23927 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a IC23927-associated disease or disorder or a pre-disposition to a IC23927-associated disease or disorder, wherein the method comprises the steps of determining IC23927 sequence information associated with the subject and based on the IC23927 sequence information, determining whether the subject has a IC23927-associated disease or disorder or a pre-disposition to a IC23927-associated disease or disorder and/or recommending a particular treatment for the disease, disorder or pre-disease condition.

The present invention further provides in an electronic system and/or in a network, a method for determining whether a subject has a IC23927-associated disease or disorder or a pre-disposition to a disease associated with a IC23927 wherein the method comprises the steps of determining IC23927 sequence information associated with the subject, and based on the IC23927 sequence information, determining whether the subject has a IC23927-associated disease or disorder or a pre-disposition to a IC23927-associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a IC23927-associated disease or disorder or a pre-disposition to a IC23927 associated disease or disorder associated with IC23927, said method comprising the steps of receiving IC23927 sequence information from the subject and/or information related thereto, receiving phenotypic information associated with the subject, acquiring information from the network corresponding to IC23927 and/or a IC23927-associated disease or disorder, and based on one or more of the phenotypic information, the IC23927 information (e.g., sequence information and/or information related thereto), and the acquired information, determining whether the subject has a IC23927-associated disease or disorder or a pre-disposition to a IC23927-associated disease or disorder (e.g., a pain disorder). The method may further comprise the step of recommending a particular treatment for the disease, disorder or pre-disease condition.

The present invention also provides a business method for determining whether a subject has a IC23927-associated disease or disorder or a pre-disposition to a IC23927-associated disease or disorder, said method comprising the steps of receiving information related to IC23927 (e.g., sequence information and/or information related thereto), receiving phenotypic information associated with the subject, acquiring information from the network related to IC23927 and/or related to a IC23927-associated disease or disorder, and based on one or more of the phenotypic information, the IC23927 information, and the acquired information, determining whether the subject has a IC23927-associated disease or disorder or a pre-disposition to a IC23927-associated disease or disorder. The method may further comprise the step of recommending a particular treatment for the disease, disorder or pre-disease condition.

The invention also includes an array comprising a IC23927 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be IC23927. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a IC23927-associated disease or disorder, progression of IC23927-associated disease or disorder, and processes, such a cellular transformation associated with the IC23927-associated disease or disorder.

The array is also useful for ascertaining the effect of the expression of a gene on the expression of other genes in the same cell or in different cells (e.g., ascertaining the effect of IC23927 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including IC23927) that could serve as a molecular target for diagnosis or therapeutic intervention.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human IC23927 cDNA

In this example, the identification and characterization of the gene encoding human IC23927 (clone Fbh23927) is described.

Isolation of the IC23927 cDNA

The invention is based, at least in part, on the discovery of a human gene encoding a novel protein, referred to herein as IC23927. The entire sequence of the human clone Fbh23927 was determined and found to contain an open reading frame termed human “IC23927.”

The nucleotide sequence encoding the human IC23927 protein is shown in FIGS. 16A-G and is set forth as SEQ ID NO:21. The protein encoded by this nucleic acid comprises about 816 amino acids and has the amino acid sequence shown in FIGS. 16A-G and set forth as SEQ ID NO:22. The coding region (open reading frame) of SEQ ID NO:21 is set forth as SEQ ID NO:23. Clone Fbh23927, comprising the coding region of human IC23927, was deposited with the American Type Culture Collection (ATCC®), 10801 University Boulevard, Manassas, Va. 20110-2209, on ______, and assigned Accession No. ______.

Analysis of the Human IC23927 Molecule

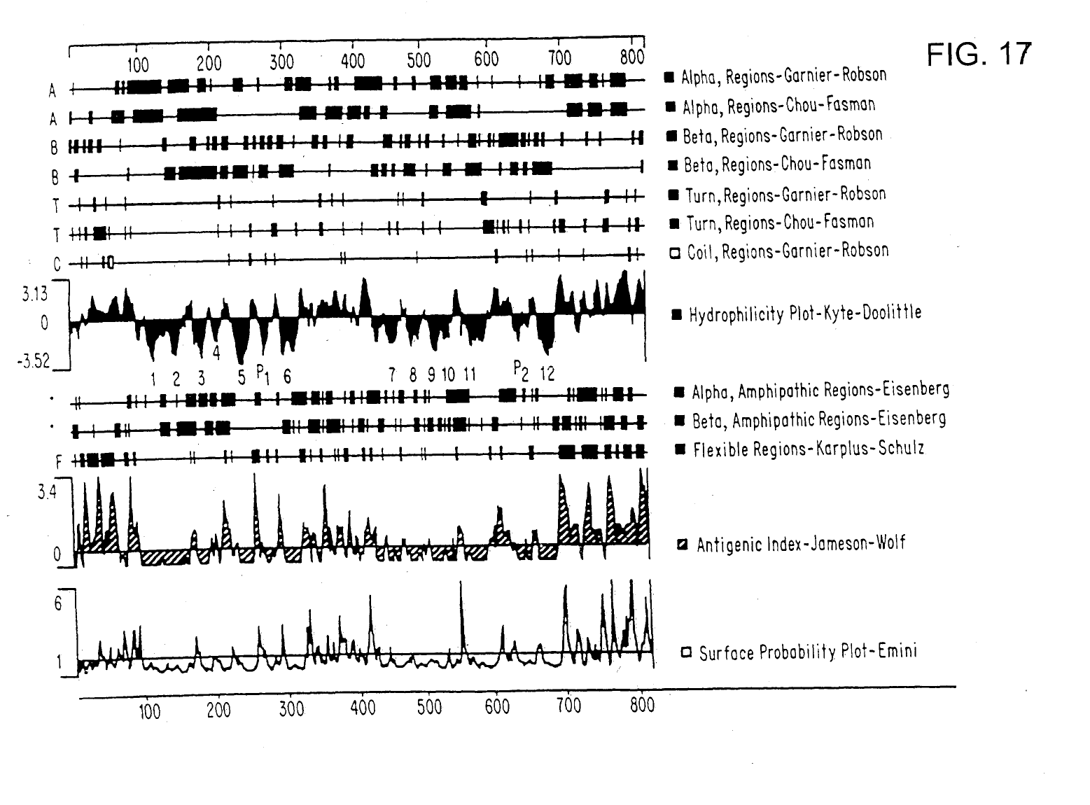

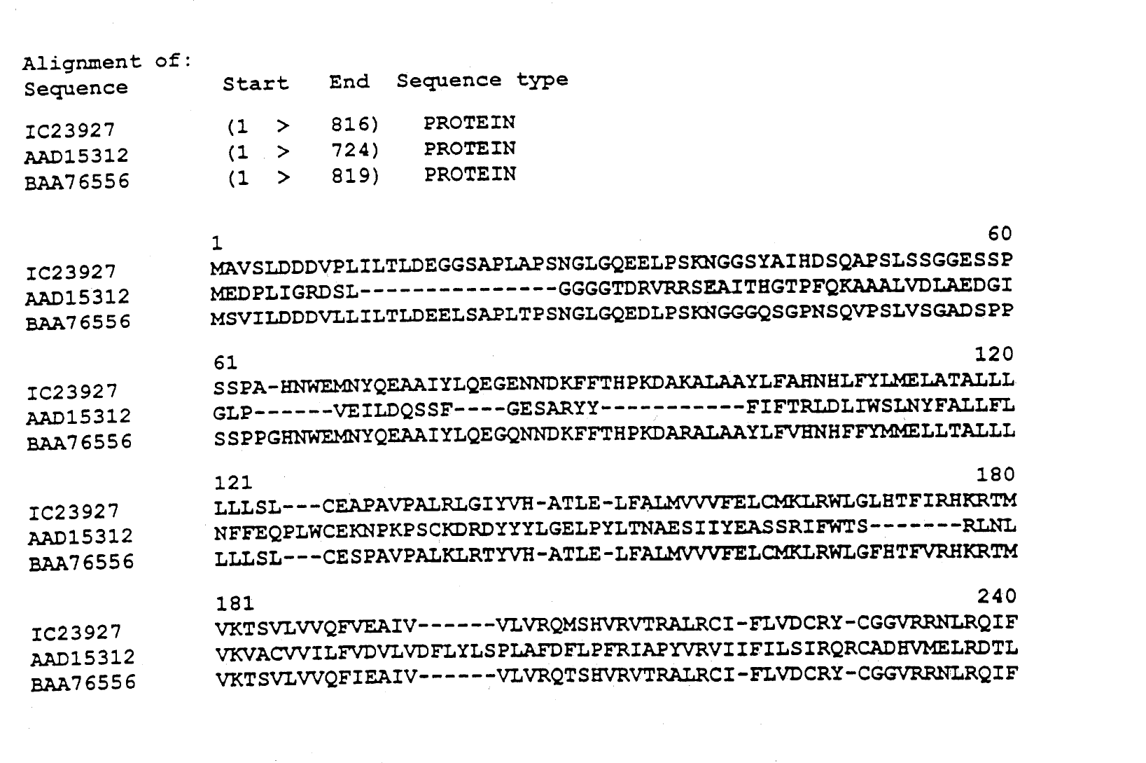

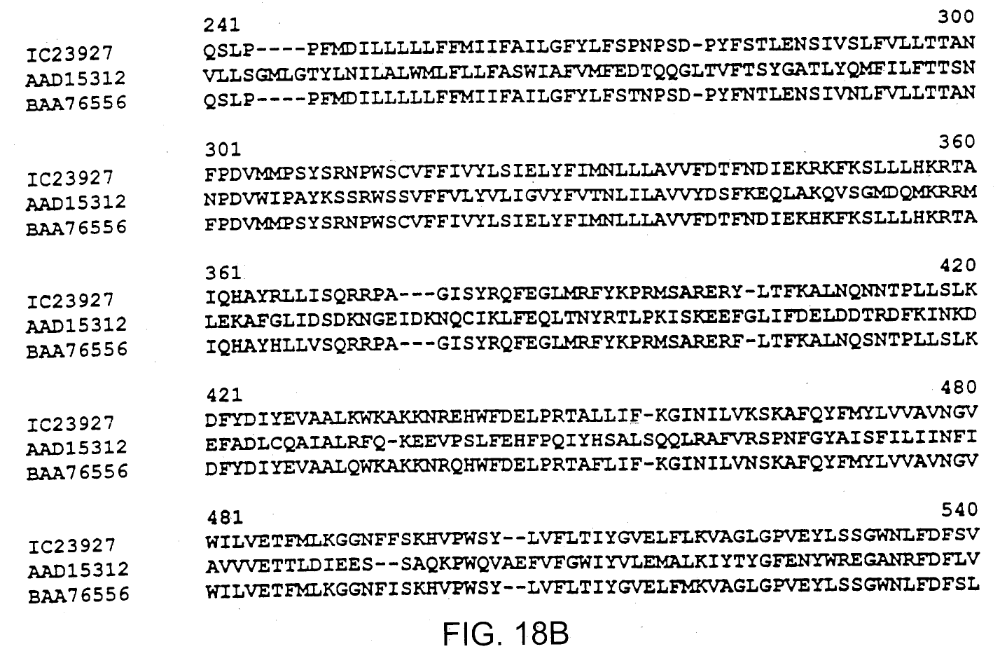

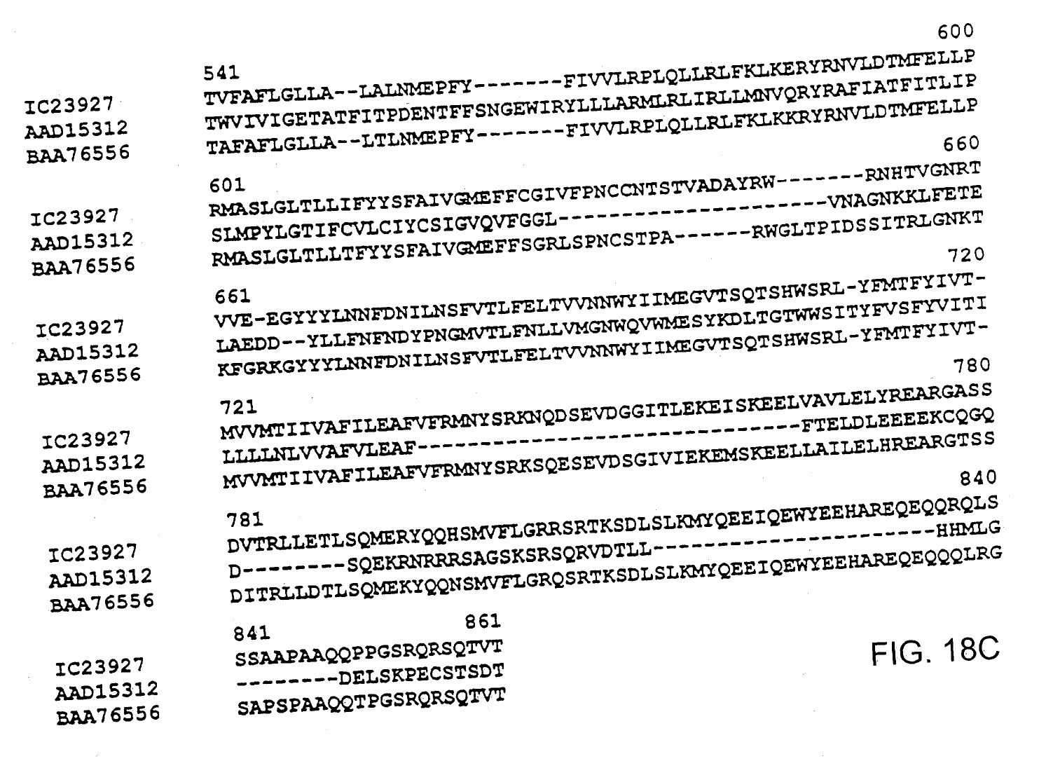

A BLAST search against the PNU database, of the nucleotide sequence of human IC23927 revealed that human IC23927 protein is homologous to a human KIAA1169 protein sequence (GenBank™ Accession Number BAA86486) over translated nucleic acid residues 411-2735. This search further revealed that human IC23927 has homology to rat voltage-gated Ca channel (GenBank™ Accession Number BAA76556) over translated nucleic acid residues 288-2735. This search further revealed that human IC23927 has homology to a putative calcium channel from Arabidopsis thaliana (GenBank™ Accession Number AAD15312) over translated nucleic acid residues 786-2357. This search further revealed that human IC23927 has homology to a voltage-gated calcium channel α1 subunit from Cyanea capillata (GenBank™ Accession Number AAC63050) over translated nucleic acid residues 1053-2060, over translated nucleic acid residues 723-1268, over translated nucleic acid residues 1596-2399, over translated nucleic acid residues 1596-2357, over translated nucleic acid residues 1947-1099, over translated nucleic acid residues 327-1301, over translated nucleic acid residues 717-1091 and over translated nucleic acid residues 2130-4779. This search further revealed that human IC23927 has homology to an N-type calcium channel α-1B cdB5 variant from Gallus gallus (GenBank™ Accession Number AAD51819) over translated nucleic acid residues 1053-2060, over translated nucleic acid residues 723-1268, over translated nucleic acid residues 1596-2399, over translated nucleic acid residues 1596-2357, over translated nucleic acid residues 1947-1099, over translated nucleic acid residues 327-1301, over translated nucleic acid residues 717-1091 and over translated nucleic acid residues 2130-4779. A Clustal alignment of the translated cDNA sequence of human IC23927 with the top two hits is provided in FIGS. 18A-C.

A MEMSAT analysis was performed, and correlated with an analysis of the hydrophilicity and surface probability of human IC23927 (FIG. 17), resulting in the identification of twelve transmembrane domains in the amino acid sequence of human IC23927 (SEQ ID NO:22) at about residues 114-128, residues 146-168, residues 178-195, residues 199-210 (or about residues 199-220), residues 233-254, residues 298-320, residues 445-465, residues 482-502 (or about residues 482-503), residues 510-532, residues 539-554, residues 570-594, and residues 666-687.

A search was also performed against the Prosite database, and resulted in the identification of 4 N-glycosylation sites at amino acid residues 599-602, 611-614, 616-619 and 695-698 of the IC23927 protein, 7 protein kinase C (PKC) phosphorylation sites at amino acid residues 351-353, 359-361, 375-377, 382-384, 395-397, 697-699 and 769-771 of the IC23927 protein, 16 casein kinase II phosphorylation sites at amino acid residues 4-7, 14-17, 54-57, 123-126, 264-267, 322-325, 375-378, 395-398, 559-562, 602-605, 618-621, 639-642, 703-706, 716-719, 745-748 and 764-767 of the IC23927 protein, 1 tyrosine kinase phosphorylation site at amino acid residues 617-625 of the IC23927 protein, 3 N-myristoylation sites at amino acid residues 39-44, 217-222 and 468-473 of the IC23927 protein, and 1 amidation site at amino acid residues 758-761 of the IC23927 protein.

A search was also performed against the PFAM database resulting in the identification of an ion transport protein domain in human IC23927 (SEQ ID NO:22) at about residues 437-686. The Hidden Markov Model for this domain has Accession No. PF00520.

A search was also performed against the ProDom database resulting in hits to the following Prodom entries including “Channel Probable Protein Ionic Transmembrane Ion Transport Voltage-Gated Calcium Channel Calcium”, “Probable GTP-Binding Protein MG384 Homolog”, and “Channel Calcium Ionic Subunit Voltage-Gated Sodium αTransmembrane L-type Ion”.

A PSORT prediction of protein localization predicted the presence of a coiled coil from about amino acid residues 768 to 796 of SEQ ID NO:22 and predicted the presence of IC23927 in at least one of the following locations: endoplasmic reticulum (score of 66.7%), mitochondria (score of 11.1%), vesicles of secretory system (score of 11.1%) and vacuolar (score of 11.1%). Accordingly, IC23927 may be localized to at least one of the endoplasmic reticulum, the mitochondria, vesicles of the secretory system, vacuoles and/or the plasma membrane.

The tissue distribution of the IC23927 homolog KIAA1699 has been determined by RT-PCR-ELISA using the following primers: forward, CCAGAGCGAGTTAATGTGTCC (SEQ ID NO:26), and reverse, AGCTGTCCCTAACCTCAATGA (SEQ ID NO:27). The mRNA has been found to be expressed at significant levels in heart, kidney, spleen, B. cerebellum and spinal cord. Radiation hybrid mapping analysis has placed the IC23927 gene on chromosome 12.

Tissue Expression Analysis of Human IC23927 mRNA Using Tagman Analysis

This example describes the tissue distribution of human IC23927 mRNA in a variety of cells and tissues, as determined using the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., brain, testis, spinal cord, skin, dorsal root ganglia, placenta, etc., and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

A human normal tissue panel indicated that human IC23927 is expressed at the highest levels in human brain, followed human kidney and testes (see Table 1, below).

TABLE 1
Tissue Expression Analysis of Human IC23927 mRNA Using
Taqman Analysis of Normal Human Tissues

| | 23927 | | | Relative |
|---|---|---|---|---|
| Tissue Type | Mean | β 2 Mean | δ Ct | Expression |
| Adrenal Gland | 31.12 | 18.89 | 12.23 | 0.21 |
| Brain | 29.50 | 20.37 | 9.14 | 1.78 |
| Heart | 31.92 | 18.95 | 12.97 | 0.13 |
| Kidney | 27.63 | 18.07 | 9.56 | 1.32 |
| Liver | 34.13 | 19.16 | 14.97 | 0.03 |
| Lung | 33.00 | 16.96 | 16.04 | 0.01 |
| Mammary Gland | 32.04 | 17.77 | 14.27 | 0.05 |
| Placenta | 30.09 | 18.59 | 11.50 | 0.35 |
| Prostate | 31.83 | 18.48 | 13.35 | 0.10 |
| Salivary Gland | 31.21 | 19.09 | 12.12 | 0.22 |
| Muscle | 32.02 | 20.96 | 11.07 | 0.47 |
| Sm. Intestine | 31.49 | 18.36 | 13.13 | 0.11 |
| Spleen | 33.17 | 16.58 | 16.60 | 0.01 |
| Stomach | 33.00 | 18.57 | 14.43 | 0.05 |
| Teste | 30.74 | 20.07 | 10.67 | 0.61 |
| Thymus | 33.44 | 18.42 | 15.02 | 0.03 |
| Trachea | 32.72 | 19.03 | 13.69 | 0.08 |
| Uterus | 34.33 | 18.63 | 15.70 | 0.02 |
| Spinal Cord | 31.91 | 18.98 | 12.93 | 0.13 |
| Skin | 33.18 | 17.17 | 16.01 | 0.02 |
| dorsal root gang. | 33.41 | 19.27 | 14.14 | 0.06 |

This expression pattern was confirmed in further experimentation using the following panel of samples (MK=monkey samples).

TABLE 2
Tissue Expression Analysis of Human IC23927 mRNA Using
Taqman Analysis of Normal Human Tissues

| | 23297 | | | Relative |
|---|---|---|---|---|
| Tissue Type | avg. | HK avg. | δδ Ct | Expression |
| MK cortex | 40 | 22.35 | 17.485 | 0.00545118 |
| MK DRG | 40 | 19.405 | 20.43 | 0.00070788 |
| MK spinal cord | 40 | 21.175 | 18.66 | 0.00241424 |
| MK sciatic nerve | 40 | 19.14 | 20.695 | 0.00058909 |
| MK kidney | 40 | 19.4 | 20.435 | 0.00070543 |
| MK hairy skin | 40 | 20.51 | 19.325 | 0.00152263 |
| MK heart LV | 40 | 19.995 | 19.84 | 0.00106553 |
| MK gastro muscle | 40 | 21.065 | 18.77 | 0.00223701 |
| MK liver | 40 | 20.67 | 19.165 | 0.00170122 |
| Hu. Brain | 28.38 | 19.6 | 8.615 | 2.5505074 |
| Hu. Spinal cord | 33.415 | 18.755 | 14.495 | 0.04330821 |
| Hu. Heart | 36.24 | 18.42 | 17.655 | 0.00484524 |
| Hu. Kidney | 29.41 | 18.64 | 10.605 | 0.6420619 |
| Hu. Liver | 39.2 | 18.98 | 20.055 | 0.000918 |
| Hu. Lung | 40 | 16.39 | 23.445 | 8.76E-05 |
| NTC | 40 | 39.835 | 0 | 1000 |
| NTC | 40 | 40 | -0.165 | 1121.16608 |

Moreover, expression in a more detailed panel of normal and disease samples confirmed this pattern of expression.

TABLE 3
Tissue Expression Analysis of Human IC23927 mRNA Using
Taqman Analysis of Normal and Diseased Human Tissues

| | 23927 | | | Relative |
|---|---|---|---|---|
| Tissue Type | Mean | β 2 Mean | ∂∂ Ct | Expression |
| Artery normal | 27.25 | 24.09 | 3.16 | 111.8781 |
| Aorta diseased | 28.18 | 23.64 | 4.54 | 42.9857 |
| Vein normal | 27.48 | 20.88 | 6.61 | 10.273 |
| Coronary SMC | 28.45 | 24.31 | 4.14 | 56.5237 |
| HUVEC | 27.72 | 22.53 | 5.18 | 27.489 |
| Hemangioma | 26.58 | 21.48 | 5.11 | 29.0564 |
| Heart normal | 26.3 | 21.56 | 4.74 | 37.4212 |
| Heart CHF | 24.27 | 20.66 | 3.6 | 82.4692 |
| Kidney | 24.45 | 21.45 | 3 | 124.5675 |
| Skeletal Muscle | 28.1 | 23.59 | 4.51 | 43.8889 |
| Adipose normal | 28.59 | 22.5 | 6.09 | 14.68 |
| Pancreas | 27.49 | 23.11 | 4.38 | 48.0273 |
| primary osteoblasts | 28.95 | 22.13 | 6.83 | 8.82 |
| Osteoclasts (diff) | 26.73 | 18.83 | 7.89 | 4.2011 |
| Skin normal | 28.93 | 23.22 | 5.71 | 19.1038 |
| Spinal cord normal | 27.38 | 22.89 | 4.49 | 44.6561 |
| Brain Cortex normal | 26.16 | 23.97 | 2.19 | 218.3932 |
| Brain Hypothalamus normal | 27.28 | 23.16 | 4.13 | 57.3128 |
| Nerve | 27.48 | 23.97 | 3.52 | 87.1715 |
| DRG (Dorsal Root Ganglion) | 26.84 | 23.48 | 3.36 | 97.3956 |
| Breast normal | 26.95 | 22.26 | 4.69 | 38.7409 |
| Breast tumor | 27.41 | 22.27 | 5.13 | 28.5572 |
| Ovary normal | 25.98 | 21.58 | 4.39 | 47.5306 |
| Ovary Tumor | 27.43 | 21.23 | 6.2 | 13.6024 |
| Prostate Normal | 27.5 | 22.61 | 4.88 | 33.9605 |
| Prostate Tumor | 26.7 | 22 | 4.71 | 38.3402 |
| Salivary glands | 26.64 | 21.89 | 4.75 | 37.1627 |
| Colon normal | 26.42 | 19.7 | 6.72 | 9.4859 |
| Colon Tumor | 25.26 | 20.25 | 5 | 31.1419 |
| Lung normal | 26.56 | 19.64 | 6.92 | 8.2294 |
| Lung tumor | 25.9 | 22.06 | 3.84 | 70.0729 |
| Lung COPD | 25.91 | 22.78 | 3.13 | 113.8337 |
| Colon IBD | 25.97 | 18.73 | 7.24 | 6.6152 |
| Liver normal | 27.14 | 21.02 | 6.13 | 14.3282 |
| Liver fibrosis | 28.18 | 22.7 | 5.47 | 22.5614 |
| Spleen normal | 27.06 | 20.97 | 6.09 | 14.6293 |
| Tonsil normal | 24.31 | 19.09 | 5.22 | 26.7373 |
| Lymph node normal | 26.56 | 20.39 | 6.17 | 13.8401 |
| Small intestine normal | 27.9 | 21.4 | 6.5 | 11.0485 |
| Skin-Decubitus | 28.05 | 23.41 | 4.63 | 40.2463 |
| Synovium | 28.19 | 21.06 | 7.13 | 7.1146 |
| BM-MNC | 29.26 | 20.35 | 8.91 | 2.0788 |
| Activated PBMC | 27.6 | 19.38 | 8.22 | 3.3538 |
| Neutrophils | 27.66 | 20.59 | 7.06 | 7.4943 |
| Megakaryocytes | 25.93 | 19.95 | 5.99 | 15.7337 |
| Erythroid | 26.68 | 22.91 | 3.77 | 73.3022 |

### Example 2

### Expression of Recombinant IC23927 Protein in Bacterial Cells

In this example, IC23927 is expressed as a recombinant glutathione-S-transferase (GST) fusion polypeptide in E. coli and the fusion polypeptide is isolated and characterized. Specifically, IC23927 is fused to GST and this fusion polypeptide is expressed in E. coli, e.g., strain PEB199. Expression of the GST-IC23927 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expession of Recombinant IC23927 Protein in COS Cells

To express the IC23927 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire IC23927 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the IC23927 DNA sequence is amplified by PCR using two primers. The 5′ primer contains the restriction site of interest followed by approximately twenty nucleotides of the IC23927 coding sequence starting from the initiation codon; the 3′ end sequence contains complementary sequences to the other restriction site of interest, a translation stop codon, the HA tag or FLAG tag and the last 20 nucleotides of the IC23927 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the IC23927 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the IC23927-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the IC23927 polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the IC23927 coding sequence is cloned directly into the polylinker of the pCDNA/Amp vector using the appropriate restriction sites. The resulting plasmid is transfected into COS cells in the manner described above, and the expression of the IC23927 polypeptide is detected by radiolabeling and immunoprecipitation using an IC23927 specific monoclonal antibody.

IV. 12303, A NOVEL HUMAN TWIK MOLECULE AND USES THEREOF

### Background of the Invention

Potassium (K⁺) channels are ubiquitous proteins which are involved in the setting of the resting membrane potential as well as in the modulation of the electrical activity of cells. In excitable cells, K⁺ channels influence action potential waveforms, firing frequency, and neurotransmitter secretion (Rudy, B. (1988) Neuroscience, 25, 729-749; Hille, B. (1992) Ionic Channels of Excitable Membranes, 2nd Ed.). In non-excitable cells, they are involved in hormone secretion, cell volume regulation and potentially in cell proliferation and differentiation (Lewis et al. (1995) Annu. Rev. Immunol., 13, 623-653). Developments in electrophysiology have allowed the identification and the characterization of an astonishing variety of K⁺ channels that differ in their biophysical properties, pharmacology, regulation and tissue distribution (Rudy, B. (1988) Neuroscience, 25, 729-749; Hille, B. (1992) Ionic Channels of Excitable Membranes, 2nd Ed.). More recently, cloning efforts have shed considerable light on the mechanisms that determine this functional diversity. Furthermore, analyses of structure-function relationships have provided an important set of data concerning the molecular basis of the biophysical properties (selectivity, gating, assembly) and the pharmacological properties of cloned K⁺ channels.

Functional diversity of K⁺ channels arises mainly from the existence of a great number of genes coding for pore-forming subunits, as well as for other associated regulatory subunits. Two main structural families of pore-forming subunits have been identified. The first one consists of subunits with a conserved hydrophobic core containing six transmembrane domains (TMDs). These K⁺ channel α subunits participate in the formation of outward rectifier voltage-gated (Kv) and Ca²⁺-dependent K⁺ channels. The fourth TMD contains repeated positive charges involved in the voltage gating of these channels and hence in their outward rectification (Logothetis et al. (1992) Neuron, 8, 531-540; Bezanilla et al. (1994) Biophys. J. 66, 1011-1021).

The second family of pore-forming subunits have only two TMDs. They are essential subunits of inward-rectifying (IRK), G-protein-coupled (GIRK) and ATP-sensitive (K_{ATP}) K⁺ channels. The inward rectification results from a voltage-dependent block by cytoplasmic Mg²⁺ and polyamines (Matsuda, H. (1991) Annu. Rev. Physiol., 53, 289-298). A conserved domain, called the P domain, is present in all members of both families (Pongs, O. (1993) J. Membr. Biol., 136, 1-8; Heginbotham et al. (1994) Biophys. J. 66,1061-1067; Mackinnon, R. (1995) Neuron, 14, 889-892; Pascual et al., (1995) Neuron., and 14, 1055-1063). This domain is an essential element of the aqueous K⁺-selective pore. In both groups, the assembly of four subunits is necessary to form a functional K⁺ channel (Mackinnon, R. (1991) Nature, 350, 232-235; Yang et al., (1995) Neuron, 15, 1441-1447.

In both six TMD and two TMD pore-forming subunit families, different subunits coded by different genes can associate to form heterotetramers with new channel properties (Isacoff et al., (1990) Nature, 345, 530-534). A selective formation of heteropolymeric channels may allow each cell to develop the best K⁺ current repertoire suited to its function. Pore-forming α subunits of Kv channels are classified into different subfamilies according to their sequence similarity (Chandy et al. (1993) Trends Pharmacol. Sci., 14: 434). Tetramerization is believed to occur preferentially between members of each subgroup (Covarrubias et al. (1991) Neuron, 7, 763-773). The domain responsible for this selective association is localized in the N-terminal region and is conserved between members of the same subgroup. This domain is necessary for hetero- but not homo-multimeric assembly within a subfamily and prevents co-assembly between subfamilies. Recently, pore-forming subunits with two TMDs were also shown to co-assemble to form heteropolymers (Duprat et al. (1995) Biochem. Biophys. Res. Commun., 212, 657-663. This heteropolymerization seems necessary to give functional GIRKs. IRKs are active as homopolymers but also form heteropolymers.

New structural types of K⁺ channels were identified recently in both humans and yeast. These channels have two P domains in their functional subunit instead of only one (Ketchum et al. (1995) Nature, 376, 690-695; Lesage et al. (1996) J. Biol. Chem, 271, 4183-4187; Lesage et al. (1996) EMBO J., 15, 1004-1011; Reid et al. (1996) Receptors Channels 4, 51-62). The human channel called TWIK-1, has four TMDs. TWIK-1 is expressed widely in human tissues and is particularly abundant in the heart and the brain. TWIK-1 currents are time independent and inwardly rectifying. These properties suggest that TWIK-1 channels are involved in the control of the background K⁺ membrane conductance (Lesage et al. (1996) EMBO J., 15, 1004-1011).

### Summary of the Invention

The present invention is based, at least in part, on the discovery of novel members of the TWIK (for Tandem of P domains in a Weak Inward rectifying K⁺ channel)-like family of potassium channels, referred to herein as TWIK-8 nucleic acid and protein molecules. The TWIK-8 molecules of the present invention are useful as targets for developing modulating agents to regulate a variety of cellular processes. Accordingly, in one aspect, this invention provides isolated nucleic acid molecules encoding TWIK-8 proteins or biologically active portions thereof, as well as nucleic acid fragments suitable as primers or hybridization probes for the detection of TWIK-8-encoding nucleic acids.

In one embodiment, a TWIK-8 nucleic acid molecule of the invention is at least 50%, 55%, 60%, 65%, 70%, 75%, 80%, 85%, 90%, 91%, 92%, 93%, 94%, 95%, 96%, 97%, 98%, 99% or more identical to the nucleotide sequence (e.g., to the entire length of the nucleotide sequence) shown in SEQ ID NO:28 or 30 or the nucleotide sequence of the DNA insert of the plasmid deposited with ATCC as Accession Number ______, or a complement thereof. In a preferred embodiment, the isolated nucleic acid molecule includes the nucleotide sequence shown SEQ ID NO:28 or 30, or a complement thereof. In another embodiment, the nucleic acid molecule includes SEQ ID NO:30 and nucleotides 1-83 of SEQ ID NO:28. In yet another embodiment, the nucleic acid molecule includes SEQ ID NO:30 and nucleotides 1344-1408 of SEQ ID NO:28. In another preferred embodiment, the nucleic acid molecule consists of the nucleotide sequence shown in SEQ ID NO:28 or 30. In another preferred embodiment, the nucleic acid molecule includes a fragment of at least 50, 100, 150, 200, 250, 300, 350, 400, 450, 500, 550, 600, 650, 700, 750, 800, 850, 900, 950, 1000, 1050, 1100, 1150, 1200, 1250, 1300, 1350, or 1400, nucleotides (e.g., contiguous nucleotides) of the nucleotide sequence of SEQ ID NO:28 or 30, or a complement thereof.

In another embodiment, a TWIK-8 nucleic acid molecule includes a nucleotide sequence encoding a protein having an amino acid sequence sufficiently identical to the amino acid sequence of SEQ ID NO:29 or an amino acid sequence encoded by the DNA insert of the plasmid deposited with ATCC as Accession Number ______. In a preferred embodiment, a TWIK-8 nucleic acid molecule includes a nucleotide sequence encoding a protein having an amino acid sequence at least 50%, 55%, 60%, 65%, 70%, 75%, 80%, 85%, 90%, 91%, 92%, 93%, 94%, 95%, 96%, 97%, 98%, 99% or more identical to the entire length of the amino acid sequence of SEQ ID NO:29 or the amino acid sequence encoded by the DNA insert of the plasmid deposited with ATCC as Accession Number ______.

In another preferred embodiment, an isolated nucleic acid molecule encodes the amino acid sequence of human TWIK-8. In yet another preferred embodiment, the nucleic acid molecule includes a nucleotide sequence encoding a protein having the amino acid sequence of SEQ ID NO:29, or the amino acid sequence encoded by the DNA insert of the plasmid deposited with ATCC as Accession Number ______.

Another embodiment of the invention features nucleic acid molecules, preferably TWIK-8 nucleic acid molecules, which specifically detect TWIK-8 nucleic acid molecules relative to nucleic acid molecules encoding non-TWIK-8 proteins. For example, in one embodiment, such a nucleic acid molecule is at least 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, or more nucleotides in length and hybridizes under stringent conditions to a nucleic acid molecule comprising the nucleotide sequence shown in SEQ ID NO:28, the nucleotide sequence of the DNA insert of the plasmid deposited with ATCC as Accession Number ______, or a complement thereof.

In other preferred embodiments, the nucleic acid molecule encodes a naturally occurring allelic variant of a polypeptide comprising the amino acid sequence of SEQ ID NO:29 or an amino acid sequence encoded by the DNA insert of the plasmid deposited with ATCC as Accession Number ______, wherein the nucleic acid molecule hybridizes to a nucleic acid molecule comprising SEQ ID NO:28 or 30 under stringent conditions.

Another embodiment of the invention provides an isolated nucleic acid molecule which is antisense to a TWIK-8 nucleic acid molecule, e.g., the coding strand of a TWIK-8 nucleic acid molecule.

Another aspect of the invention provides a vector comprising a TWIK-8 nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably a TWIK-8 protein, by culturing in a suitable medium, a host cell, e.g., a mammalian host cell such as a non-human mammalian cell, of the invention containing a recombinant expression vector, such that the protein is produced.

Another aspect of this invention features isolated or recombinant TWIK-8 proteins and polypeptides. In one embodiment, an isolated TWIK-8 protein includes at least one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain.

In a preferred embodiment, a TWIK-8 protein includes at least one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain, and has an amino acid sequence at least about 50%, 55%, 60%, 65%, 70%, 75%, 80%, 85%, 90%, 91%, 92%, 93%, 94%, 95%, 96%, 97%, 98%, 99% or more identical to the amino acid sequence of SEQ ID NO:29, or the amino acid sequence encoded by the DNA insert of the plasmid deposited with ATCC as Accession Number ______.

In another preferred embodiment, a TWIK-8 protein includes one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain, and has a TWIK-8 activity (as described herein).

In yet another preferred embodiment, a TWIK-8 protein includes one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain, and is encoded by a nucleic acid molecule having a nucleotide sequence which hybridizes under stringent hybridization conditions to a nucleic acid molecule comprising the nucleotide sequence of SEQ ID NO:28 or 30.

In another embodiment, the invention features fragments of the protein having the amino acid sequence of SEQ ID NO:29, wherein the fragment comprises at least 15 amino acids (e.g., 15 contiguous amino acids) of the amino acid sequence of SEQ ID NO:29, or an amino acid sequence encoded by the DNA insert of the plasmid deposited with the ATCC as Accession Number ______. In another embodiment, a TWIK-8 protein has the amino acid sequence of SEQ ID NO:29.

In another embodiment, the invention features a TWIK-8 protein which is encoded by a nucleic acid molecule consisting of a nucleotide sequence at least about 50%, 55%, 60%, 65%, 70%, 75%, 80%, 85%, 90%, 91%, 92%, 93%, 94%, 95%, 96%, 97%, 98%, 99% or more identical to a nucleotide sequence of SEQ ID NO:28 or 30, or a complement thereof. This invention further features a TWIK-8 protein, which is encoded by a nucleic acid molecule consisting of a nucleotide sequence which hybridizes under stringent hybridization conditions to a nucleic acid molecule comprising the nucleotide sequence of SEQ ID NO:28 or 30, or a complement thereof.

The proteins of the present invention or portions thereof, e.g., biologically active portions thereof, can be operatively linked to a non-TWIK-8 polypeptide (e.g., heterologous amino acid sequences) to form fusion proteins. The invention further features antibodies, such as monoclonal or polyclonal antibodies, that specifically bind proteins of the invention, preferably TWIK-8 proteins. In addition, the TWIK-8 proteins or biologically active portions thereof can be incorporated into pharmaceutical compositions, which optionally include pharmaceutically acceptable carriers.

In another aspect, the present invention provides a method for detecting the presence of a TWIK-8 nucleic acid molecule, protein, or polypeptide in a biological sample by contacting the biological sample with an agent capable of detecting a TWIK-8 nucleic acid molecule, protein, or polypeptide such that the presence of a TWIK-8 nucleic acid molecule, protein or polypeptide is detected in the biological sample.

In another aspect, the present invention provides a method for detecting the presence of TWIK-8 activity in a biological sample by contacting the biological sample with an agent capable of detecting an indicator of TWIK-8 activity such that the presence of TWIK-8 activity is detected in the biological sample.

In another aspect, the invention provides a method for modulating TWIK-8 activity comprising contacting a cell capable of expressing TWIK-8 with an agent that modulates TWIK-8 activity such that TWIK-8 activity in the cell is modulated. In one embodiment, the agent inhibits TWIK-8 activity. In another embodiment, the agent stimulates TWIK-8 activity. In one embodiment, the agent is an antibody that specifically binds to a TWIK-8 protein. In another embodiment, the agent modulates expression of TWIK-8 by modulating transcription of a TWIK-8 gene or translation of a TWIK-8 mRNA. In yet another embodiment, the agent is a nucleic acid molecule having a nucleotide sequence that is antisense to the coding strand of a TWIK-8 mRNA or a TWIK-8 gene.

In one embodiment, the methods of the present invention are used to treat a subject having a disorder characterized by aberrant or unwanted TWIK-8 protein or nucleic acid expression or activity by administering an agent which is a TWIK-8 modulator to the subject. In one embodiment, the TWIK-8 modulator is a TWIK-8 protein. In another embodiment the TWIK-8 modulator is a TWIK-8 nucleic acid molecule. In yet another embodiment, the TWIK-8 modulator is a peptide, peptidomimetic, or other small molecule. In a preferred embodiment, the disorder characterized by aberrant or unwanted TWIK-8 protein or nucleic acid expression is a CNS disorder, such as a cognitive or neurodegenerative disorder. In another preferred embodiment, the disorder characterized by aberrant or unwanted TWIK-8 protein or nucleic acid expression is a cardiovascular disorder. In another preferred embodiment, the disorder characterized by aberrant or unwanted TWIK-8 protein or nucleic acid expression is a muscular disorder. In another embodiment, the disorder characterized by aberrant or unwanted TWIK-8 activity is a cell proliferation, growth, differentiation, or migration disorder. In another embodiment, the disorder characterized by aberrant or unwanted TWIK-8 activity is a pain disorder, or a disorder characterized by misregulated pain signaling mechanisms.

The present invention also provides diagnostic assays for identifying the presence or absence of a genetic alteration characterized by at least one of (i) aberrant modification or mutation of a gene encoding a TWIK-8 protein; (ii) mis-regulation of the gene; and (iii) aberrant post-translational modification of a TWIK-8 protein, wherein a wild-type form of the gene encodes a protein with a TWIK-8 activity.

In another aspect the invention provides methods for identifying a compound that binds to or modulates the activity of a TWIK-8 protein, by providing an indicator composition comprising a TWIK-8 protein having TWIK-8 activity, contacting the indicator composition with a test compound, and determining the effect of the test compound on TWIK-8 activity in the indicator composition to identify a compound that modulates the activity of a TWIK-8 protein.

Other features and advantages of the invention will be apparent from the following detailed description and claims.

### Detailed Description of the Invention

The present invention is based, at least in part, on the discovery of novel molecules, referred to herein as TWIK-8 nucleic acid and protein molecules, which are novel members of the TWIK (for Tandem of P domains in a Weak Inward rectifying K⁺ channel)-like family of potassium channels. These novel molecules are capable of, for example, modulating a potassium channel mediated activity in a cell, e.g., a neuronal cell, or a muscle cell.

As used herein, a “potassium channel” includes a protein or polypeptide which is involved in receiving, conducting, and transmitting signals in an electrically excitable or a non-electrically excitable cell, e.g., a neuronal cell, or a muscle cell (e.g., a cardiac muscle cell). Potassium channels are potassium ion selective, and can determine membrane excitability (the ability of, for example, a neuron to respond to a stimulus and convert it into an impulse). Potassium channels can also influence the resting potential of membranes, wave forms and frequencies of action potentials, and thresholds of excitation. Potassium channels are typically expressed in electrically excitable cells, e.g., neurons, muscle, endocrine, and egg cells, and may form heteromultimeric structures, e.g., composed of pore-forming α and cytoplasmic β subunits. Potassium channels may also be found in non-excitable cells (e.g., spleen cells or prostate cells), where they may play a role in, e.g., signal transduction. Examples of potassium channels include: (1) the voltage-gated potassium channels, (2) the ligand-gated potassium channels, e.g., neurotransmitter-gated potassium channels, and (3) cyclic-nucleotide-gated potassium channels. Voltage-gated and ligand-gated potassium channels are expressed in the brain, e.g., in brainstem monoaminergic and forebrain cholinergic neurons, where they are involved in the release of neurotransmitters, or in the dendrites of hippocampal and neocortical pyramidal cells, where they are involved in the processes of learning and memory formation. For a detailed description of potassium channels, see Kandel E. R.. et al., Principles of Neural Science, second edition, (Elsevier Science Publishing Co., Inc., N.Y. (1985)), the contents of which are incorporated herein by reference. As the TWIK-like proteins of the present invention may modulate potassium channel mediated activities, they may be useful for developing novel diagnostic and therapeutic agents for potassium channel associated disorders.

As used herein, a “potassium channel associated disorder” includes a disorder, disease or condition which is characterized by a misregulation of a potassium channel mediated activity. Potassium channel associated disorders can detrimentally affect conveyance of sensory impulses from the periphery to the brain and/or conductance of motor impulses from the brain to the periphery; integration of reflexes; interpretation of sensory impulses; and emotional, intellectual (e.g., learning and memory), or motor processes.

Examples of potassium channel associated disorders include CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, movement disorders, progressive supranuclear palsy, epilepsy, AIDS related dementia, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further examples of potassium channel associated disorders include cardiac-related disorders. Cardiovascular system disorders in which the TWIK-8 molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia. TWIK-8-mediated or related disorders also include disorders of the musculoskeletal system such as paralysis and muscle weakness, e.g., ataxia, myotonia, and myokymia.

Other examples of potassium channel-associated disorders include pain disorders. Pain disorders include those disorders that affect pain signaling mechanisms. As used herein, the term “pain signaling mechanisms” includes the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter sensory neurons. These sensory neurons transmit the information to the central nervous system, evoking a perception of pain or discomfort and initiating appropriate protective reflexes. The TWIK-8 molecules of the present invention may be present on these sensory neurons and, thus, may be involved in detecting these noxious chemical, mechanical, or thermal stimuli and transducing this information into membrane depolarization events. Thus, the TWIK-8 molecules by participating in pain signaling mechanisms, may modulate pain elicitation and act as targets for developing novel diagnostic targets and therapeutic agents to control pain. Examples of pain disorders include headache posttherapeutic neuralgia, diabetic neuropathy, postmastectomy pain syndrome, stump pain, reflex sympathetic dystrophy, trigeminal neuralgia, neuropathic pain, orofacial neuropathic pain, osteoarthritis, arthritis, e.g., rheumatoid arthritis, fibromyalgia syndrome, tension myalgia, Guillian-Barre syndrome, Meralgia paraesthetica, burning mouth syndrome, fibrocitis, myofascial pain syndrome, idiopathic pain disorder, temporomandibular joint syndrome, atypical odontalgia, loin pain, haematuria syndrome, non-cardiac chest pain, back pain, chronic nonspecific pain, pain associated with surgery, psychogenic pain, tooth pain, musculoskeletal pain disorder, chronic pelvic pain, nonorganic chronic headache, tension-type headache, cluster headache, migraine, complex regional pain syndrome, vaginismus, nerve trunk pain, somatoform pain disorder, cyclical mastalgia, chronic fatigue syndrome, multiple somatization syndrome, chronic pain disorder, cancer pain, somatization disorder, Syndrome X, facial pain, idiopathic pain disorder, posttraumatic rheumatic pain modulation disorder (fibrositis syndrome), hyperalgesia, and Tangier disease.

Potassium channel disorders also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The TWIK-8 molecules of the present invention are involved in signal transduction mechanisms, which are known to be involved in cellular growth, differentiation, and migration processes. Thus, the TWIK-8 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; and hematopoietic and/or myeloproliferative disorders.

TWIK-8-associated or related disorders also include disorders of tissues in which TWIK-8 protein is expressed, e.g., cortex, hypothalamus and dorsal root ganglia.

As used herein, a “potassium channel mediated activity” includes an activity which involves a potassium channel, e.g., a potassium channel in a neuronal cell, or a muscle cell (e.g., a cardiac muscle cell), associated with receiving, conducting, and transmitting signals in, for example, the nervous system. Potassium channel mediated activities include release of neurotransmitters, e.g., dopamine or norepinephrine, from cells, e.g., neuronal cells; modulation of resting potential of membranes, wave forms and frequencies of action potentials, and thresholds of excitation; and modulation of processes such as integration of sub-threshold synaptic responses, the conductance of back-propagating action potentials in, for example, neuronal cells or muscle cells, participation in signal transduction pathways, and participation in nociception.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., monkey proteins. Members of a family may also have common functional characteristics.

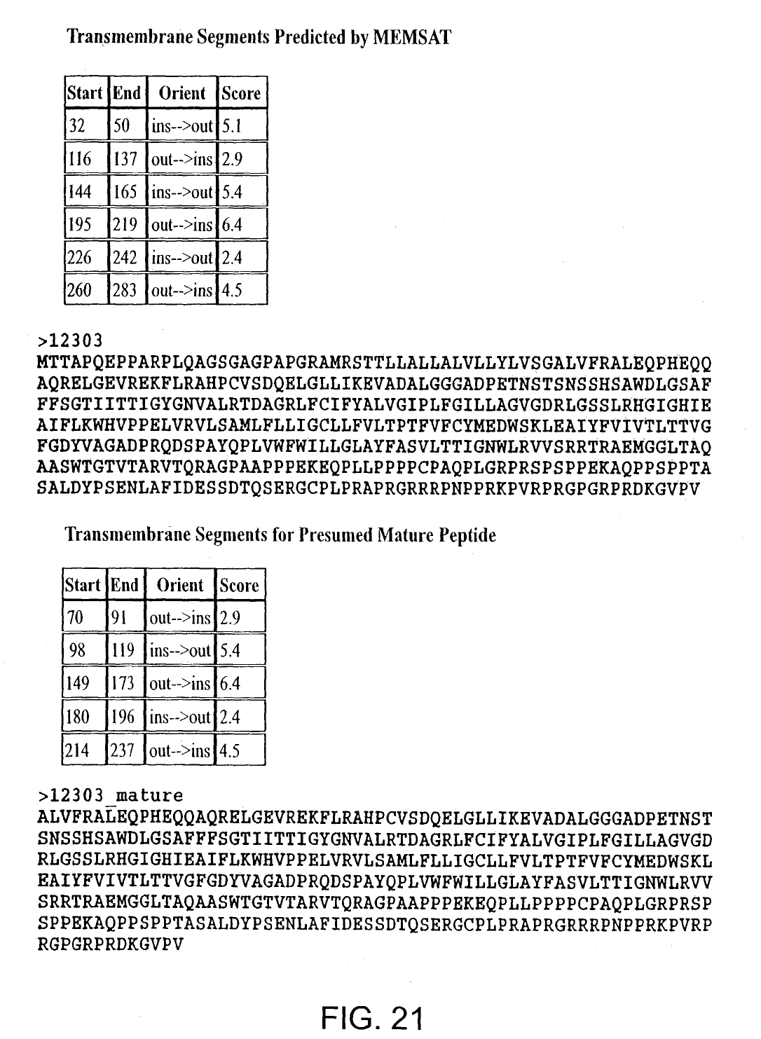

For example, the family of TWIK-8 proteins comprises at least one “transmembrane domain” and preferably six transmembrane domains. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al. (1996) Annual Rev. Neurosci. 19: 235-263, the contents of which are incorporated herein by reference. Amino acid residues 32-50, 116-137, 144-165, 195-219, 226-242, and 260-283 of the native TWIK-8 protein, and amino acid residues 70-91, 98-119, 149-173, 180-196 and 214-237 of the mature TWIK-8 protein are predicted to comprise transmembrane domains (see FIG. 21). Accordingly, TWIK-8 proteins having at least 50-60% homology, preferably about 60-70%, more preferably about 70-80%, or about 80-90% homology with a transmembrane domain of human TWIK-8 are within the scope of the invention.

In another embodiment, a TWIK-8 molecule of the present invention is identified based on the presence of a Pore loop (P-loop). As used herein, the term “Pore loop” or “P-loop” includes an amino acid sequence of about 15-45 amino acid residues in length, preferably about 15-35 amino acid residues in length, and most preferably about 15-25 amino acid residues in length, which is involved in lining the potassium channel pore. A P-loop is typically found between transmembrane domains of potassium channels and is believed to be a major determinant of ion selectivity in potassium channels. Preferably, P-loops contain a G-[HYDROPHOBIC AMINO ACID]-G sequence, e.g., a GYG, GLG, or GFG sequence. P-loops are described in, for example, Warmke et al. (1991) Science 252:1560-1562; Zagotta W. N. et al., (1996) Annual Rev. Neurosci. 19:235-63 (Pongs, O. (1993) J. Membr. Biol., 136, 1-8; Heginbotham et al. (1994) Biophys. J. 66,1061-1067; Mackinnon, R. (1995) Neuron, and 14, 889-892; Pascual et al., (1995) Neuron., 14, 1055-1063), the contents of which are incorporated herein by reference. Amino acid residues 243-259 of the native human TWIK-8 protein, and residues 197-213 of the predicted mature human TWIK-8 protein comprise a P-loop.

In a preferred embodiment, the TWIK-8 molecules of the invention include at least one and, preferably, six transmembrane domains and at least one P-loop domain.

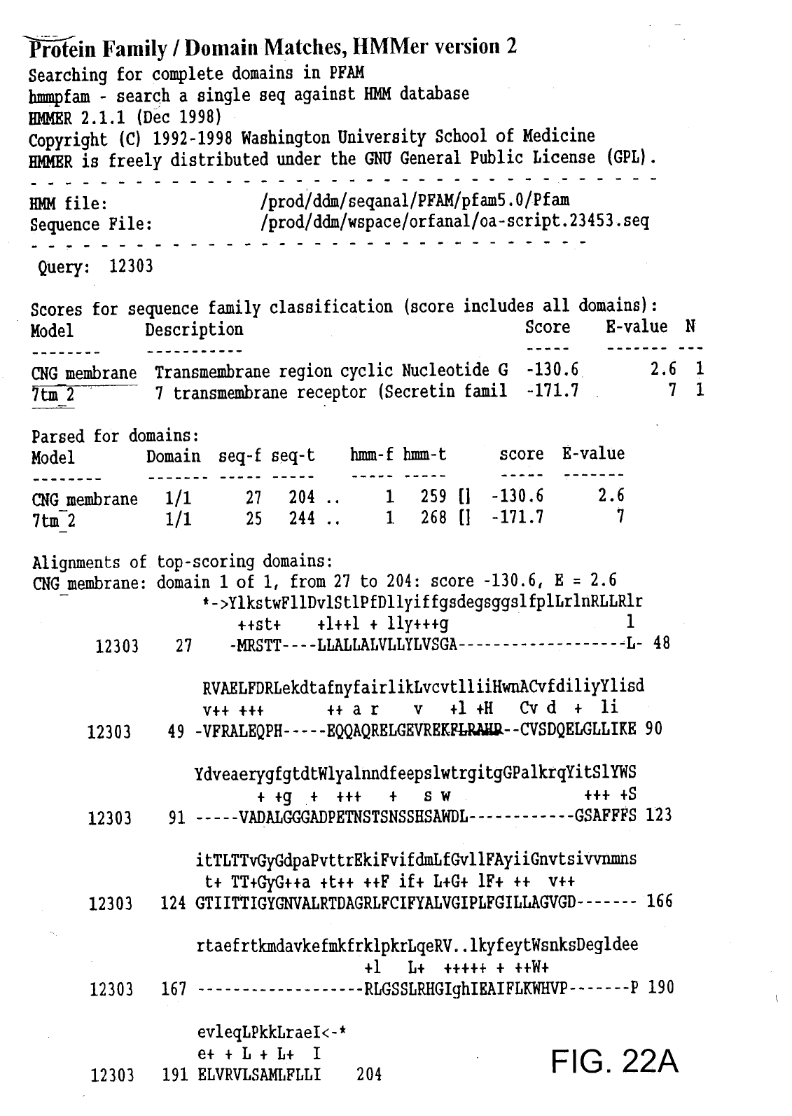

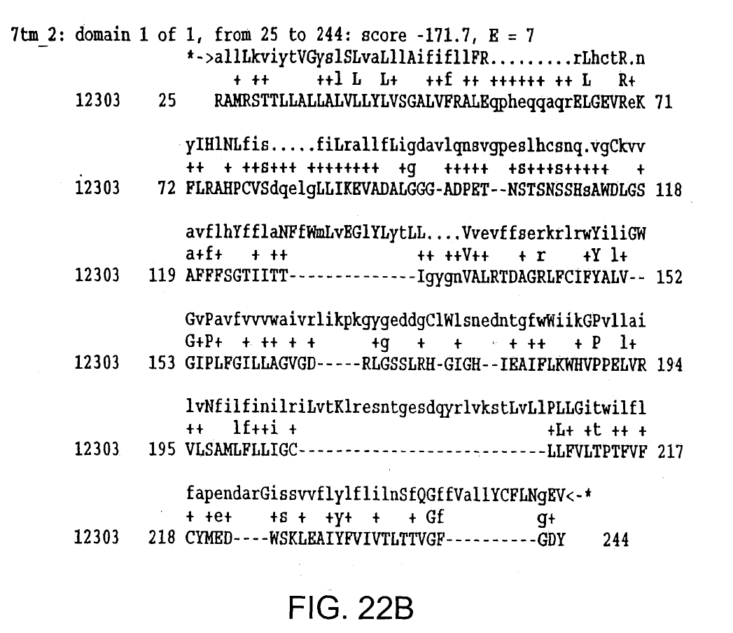

In another embodiment, a TWIK-8 molecule of the present invention is identified based on the presence of a “seven-transmembrane receptor domain” in the protein or corresponding nucleic acid molecule. Seven-transmembrane receptor domains are described, for example, in Hamann et al. (1996) Genomics 32: 144-147. As used herein, the term “seven-transmembrane receptor domain” includes a protein domain having an amino acid sequence of about 150-320 amino acid residues. Preferably, a seven-transmembrane receptor domain includes at least about 200-250, or more preferably about 220 amino acid residues. To identify the presence of a seven-transmembrane receptor domain in a TWIK-8 protein, and to make the determination that a protein of interest has a particular profile, the amino acid sequence of the protein is searched against a database of known protein domains (e.g., the HMM database). The seven-transmembrane receptor domain (HMM) has been assigned the PFAM Accession PF00002 (http://genome.wustl.edu/Pfam/html). A search was performed against the HMM database resulting in the identification of a seven-transmembrane receptor domain in the amino acid sequence of human TWIK-8 (SEQ ID NO:29) at about residues 25-244 of SEQ ID NO:29. The results of the search are set forth in FIG. 22.

In another embodiment, a TWIK-8 molecule of the present invention is identified based on the presence of a “cyclic nucleotide-gated channel domain” in the protein or corresponding nucleic acid molecule. Cyclic nucleotide-gated channel domains are described, for example, in Yau (1994) Proc. Natl. Acad. Sci. USA 91: 3481-3483. As used herein, the term “cyclic nucleotide-gated channel domain” includes a protein domain having an amino acid sequence of about 100-225 amino acid residues. Preferably, a cyclic nucleotide-gated channel domain includes at least about 150-200, or more preferably about 178 amino acid residues. To identify the presence of a cyclic nucleotide-gated channel domain in a TWIK-8 protein, and to make the determination that a protein of interest has a particular profile, the amino acid sequence of the protein is searched against a database of known protein domains (e.g., the HMM database). The cyclic nucleotide-gated channel domain (HMM) has been assigned the PFAM Accession PF00914 (http:H/genome.wustl.edu/Pfam/html). A search was performed against the HMM database resulting in the identification of a cyclic nucleotide-gated channel domain in the amino acid sequence of human TWIK-8 (SEQ ID NO:29) at about residues 27-204 of SEQ ID NO:29. The results of the search are set forth in FIG. 22.

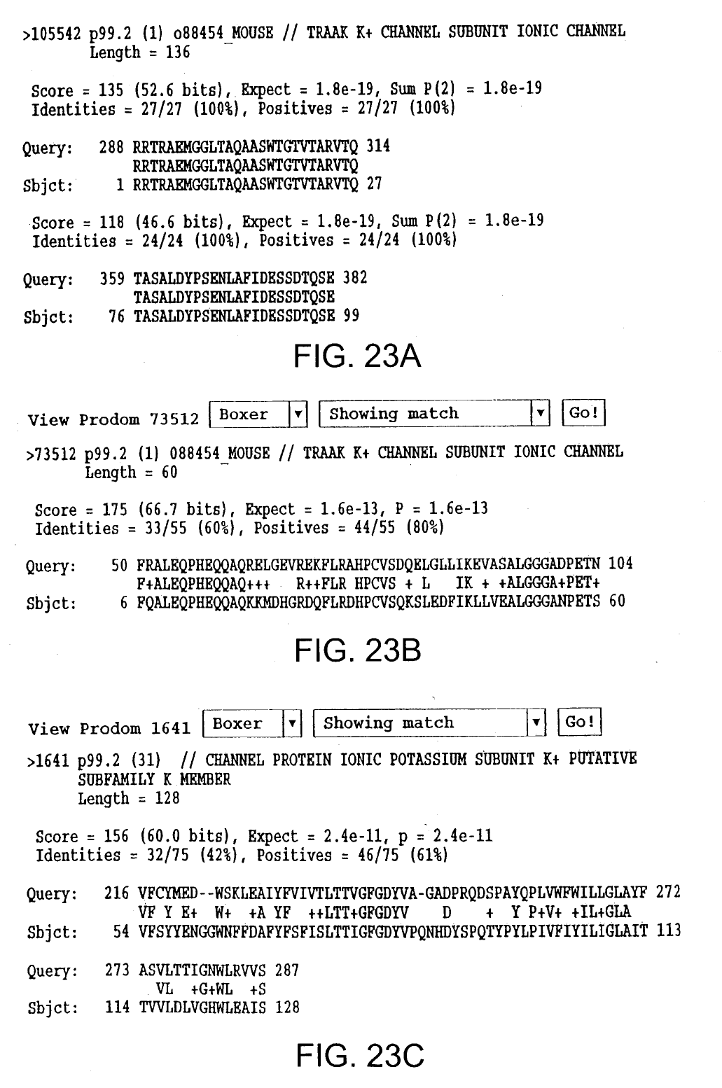

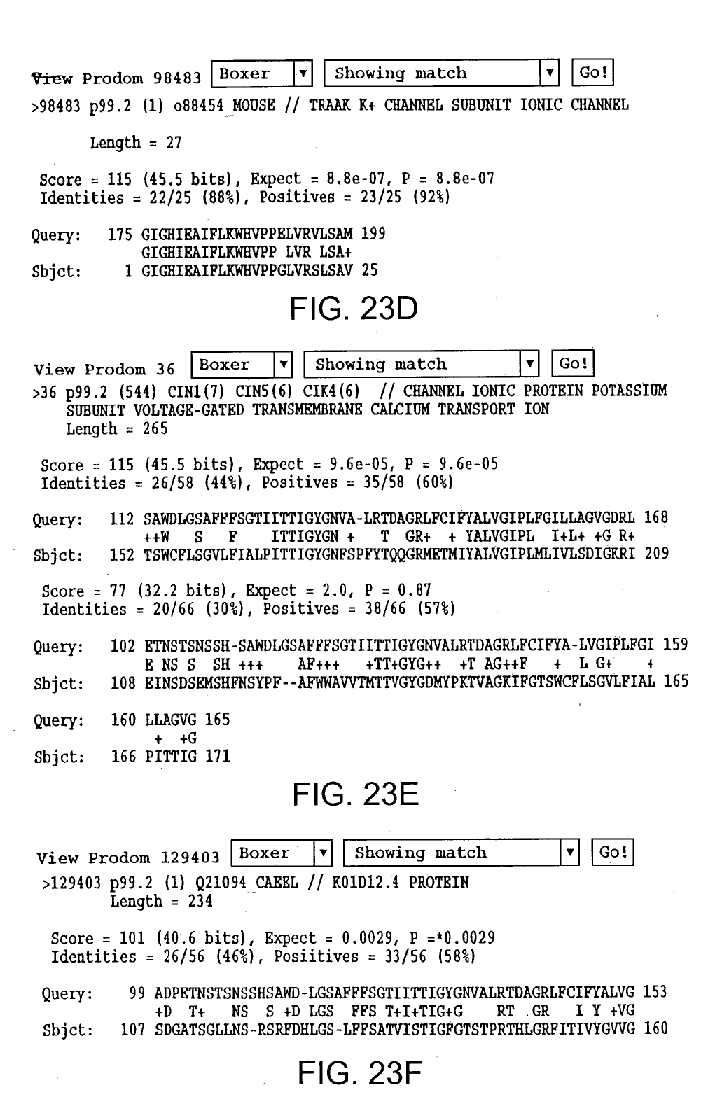

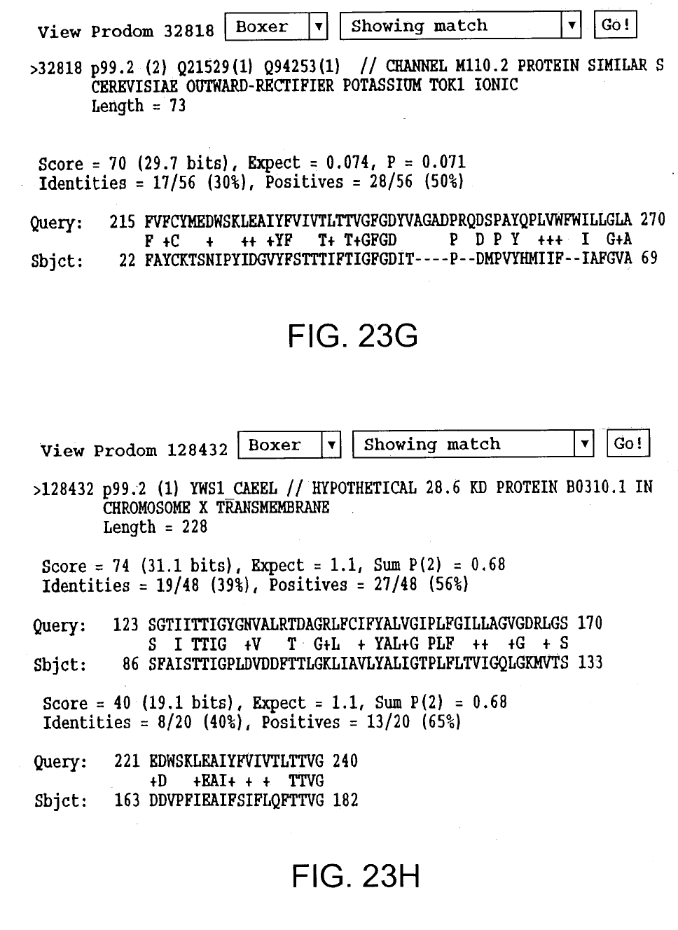

In another embodiment, a TWIK-8 molecule of the present invention is identified based on the presence of a “TRAAK potassium channel domain” in the protein or corresponding nucleic acid molecule. As used herein, the term “TRAAK potassium channel domain” includes a protein domain having an amino acid sequence of about 20-150 amino acid residues and having a bit score for the alignment of the sequence to the TRAAK potassium channel domain of at least 115-175. Preferably, a TRAAK potassium channel domain includes at least about 23-100, or more preferably about 25, 55, or 95 amino acid residues, and has a bit score for the alignment of the sequence to the TRAAK potassium channel domain of at least 20, 30, 40, 50, 60, 70, 80, 90, 100, 110 or higher. The TRAAK potassium channel domain has been assigned ProDom entries 73512, 98483, and 105542. To identify the presence of a TRAAK potassium channel domain in a TWIK-8 protein, and to make the determination that a protein of interest has a particular profile, the amino acid sequence of the protein is searched against a database of known protein domains (e.g., the ProDom database) using the default parameters (available at http://www.toulouse.inra.fr/prodom.html). A search was performed against the ProDom database resulting in the identification of a TRAAK potassium channel domains in the amino acid sequence of human TWIK-8 (SEQ ID NO:29) at about residues 50-104, 175-199, and 288-382 of SEQ ID NO:29. The results of the search are set forth in FIGS. 23A-H.

In another embodiment, a TWIK-8 molecule of the present invention is identified based on the presence of a “potassium channel protein domain” in the protein or corresponding nucleic acid molecule. As used herein, the term “potassium channel protein domain” includes a protein domain having an amino acid sequence of about 20-100 amino acid residues and having a bit score for the alignment of the sequence to the potassium channel protein domain of at least 101. Preferably, a potassium channel protein domain includes at least about 40-75, or more preferably about 55 amino acid residues, and has a bit score for the alignment of the sequence to the potassium channel protein domain of at least 20, 30, 40, 50, 60, 70, 80, 90, 100, or higher. The potassium channel protein domain has been assigned ProDom entry 129403. To identify the presence of a potassium channel protein domain in a TWIK-8 protein, and to make the determination that a protein of interest has a particular profile, the amino acid sequence of the protein is searched against a database of known protein domains (e.g., the ProDom database) using the default parameters (available at http://www.toulouse.inra.fr/prodom.html). A search was performed against the ProDom database resulting in the identification of a potassium channel protein domain in the amino acid sequence of human TWIK-8 (SEQ ID NO:29) at about residues 99-153 of SEQ ID NO:29. The results of the search are set forth in FIGS. 23A-H.

In another embodiment, a TWIK-8 molecule of the present invention is identified based on the presence of a “voltage-gated potassium channel domain” in the protein or corresponding nucleic acid molecule. As used herein, the term “voltage-gated potassium channel domain” includes a protein domain having an amino acid sequence of about 20-100 amino acid residues and having a bit score for the alignment of the sequence to the potassium channel protein domain of at least 115. Preferably, a voltage-gated potassium channel domain includes at least about 40-75, or more preferably about 55 amino acid residues, and has a bit score for the alignment of the sequence to the voltage-gated potassium channel domain of at least 20, 30, 40, 50, 60, 70, 80, 90, 100, 110 or higher. The voltage-gated potassium channel domain has been assigned ProDom entry 36. To identify the presence of a voltage-gated potassium channel domain in a TWIK-8 protein, and to make the determination that a protein of interest has a particular profile, the amino acid sequence of the protein is searched against a database of known protein domains (e.g., the ProDom database) using the default parameters (available at http:H/www.toulouse.inra.fr/prodom.html). A search was performed against the ProDom database resulting in the identification of a voltage-gated potassium channel domain in the amino acid sequence of human TWIK-8 (SEQ ID NO:29) at about residues 102-168 of SEQ ID NO:29. The results of the search are set forth in FIGS. 23A-H.

In another embodiment, a TWIK-8 molecule of the present invention is identified based on the presence of an “outward-rectifier TOK1 potassium channel domain” in the protein or corresponding nucleic acid molecule. As used herein, the term “outward-rectifier TOK1 potassium channel domain” includes a protein domain having an amino acid sequence of about 25-100 amino acid residues and having a bit score for the alignment of the sequence to the outward-rectifier TOK1 potassium channel domain of at least 70. Preferably, an outward-rectifier TOK1 potassium channel domain includes at least about 40-75, or more preferably about 56 amino acid residues, and has a bit score for the alignment of the sequence to the aoutward-rectifier TOK1 potassium channel domain of at least 20, 30, 40, 50, 60, or higher. The outward-rectifier TOK1 potassium channel domain has been assigned ProDom entry 32818. To identify the presence of an outward-rectifier TOK1 potassium channel domain in a TWIK-8 protein, and to make the determination that a protein of interest has a particular profile, the amino acid sequence of the protein is searched against a database of known protein domains (e.g., the ProDom database) using the default parameters (available at http://www.toulouse.inra.fr/prodom.html). A search was performed against the ProDom database resulting in the identification of an outward-rectifier TOK1 potassium channel domain in the amino acid sequence of human TWIK-8 (SEQ ID NO:29) at about residues 215-270 of SEQ ID NO:29. The results of the search are set forth in FIGS. 23A-H.

In another embodiment, a TWIK-8 molecule of the present invention is identified based on the presence of a “potassium channel subunit domain” in the protein or corresponding nucleic acid molecule. As used herein, the term “potassium channel subunit domain” includes a protein domain having an amino acid sequence of about 25-125 amino acid residues and having a bit score for the alignment of the sequence to the potassium channel subunit domain of at least 156. Preferably, a potassium channel subunit domain includes at least about 40-100, or more preferably about 72 amino acid residues, and has a bit score for the alignment of the sequence to the potassium channel subunit domain of at least 20, 30, 40, 50, 60, or higher. The potassium channel subunit domain has been assigned ProDom entry 1641. To identify the presence of a potassium channel subunit domain in a TWIK-8 protein, and to make the determination that a protein of interest has a particular profile, the amino acid sequence of the protein is searched against a database of known protein domains (e.g., the ProDom database) using the default parameters (available at http://www.toulouse.inra.fr/prodom.html). A search was performed against the ProDom database resulting in the identification of a potassium channel subunit domain in the amino acid sequence of human TWIK-8 (SEQ ID NO:29) at about residues 216-287 of SEQ ID NO:29. The results of the search are set forth in FIGS. 23A-H.

Isolated proteins of the present invention, preferably TWIK-8 proteins, have an amino acid sequence sufficiently identical to the amino acid sequence of SEQ ID NO:29 or are encoded by a nucleotide sequence sufficiently identical to SEQ ID NO:28 or 30. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, an “TWIK-8 activity”, “biological activity of TWIK-8” or “functional activity of TWIK-8”, refers to an activity exerted by a TWIK-8 protein, polypeptide or nucleic acid molecule on a TWIK-8 responsive cell or tissue, or on a TWIK-8 protein substrate, as determined in vivo, or in vitro, according to standard techniques. In one embodiment, a TWIK-8 activity is a direct activity, such as an association with a TWIK-8-target molecule. As used herein, a “target molecule” or “binding partner” is a molecule with which a TWIK-8 protein binds or interacts in nature, such that TWIK-8-mediated function is achieved. A TWIK-8 target molecule can be a non-TWIK-8 molecule or a TWIK-8 protein or polypeptide of the present invention. In an exemplary embodiment, a TWIK-8 target molecule is a TWIK-8 ligand, e.g., a potassium channel pore-forming subunit or a potassium channel ligand. Alternatively, a TWIK-8 activity is an indirect activity, such as a cellular signaling activity mediated by interaction of the TWIK-8 protein with a TWIK-8 ligand. The biological activities of TWIK-8 are described herein. For example, the TWIK-8 proteins of the present invention can have one or more of the following activities: (1) interacting with a non-TWIK protein molecule; (2) activating a TWIK-dependent signal transduction pathway; (3) modulating the release of neurotransmitters; (4) modulating membrane excitability; (5) influencing the resting potential of membranes, wave forms and frequencies of action potentials, and thresholds of excitation, (6) modulating processes which underlie learning and memory, such as integration of sub-threshold synaptic responses and the conductance of back-propagating action potentials, and (7) mediating nociception.

Accordingly, another embodiment of the invention features isolated TWIK-8 proteins and polypeptides having a TWIK-8 activity. Preferred proteins are TWIK-8 proteins having at least one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain, and, preferably, a TWIK-8 activity.

Additional preferred proteins have at least one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain, and are, preferably, encoded by a nucleic acid molecule having a nucleotide sequence which hybridizes under stringent hybridization conditions to a nucleic acid molecule comprising the nucleotide sequence of SEQ ID NO:28 or 30.

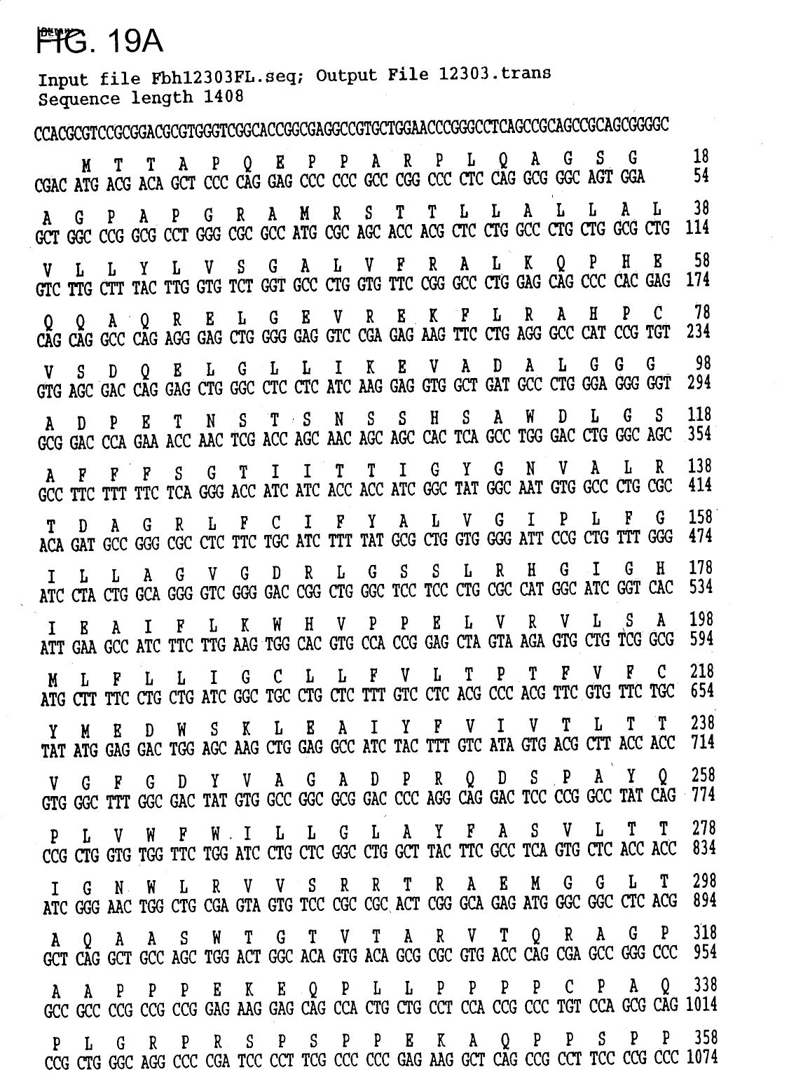

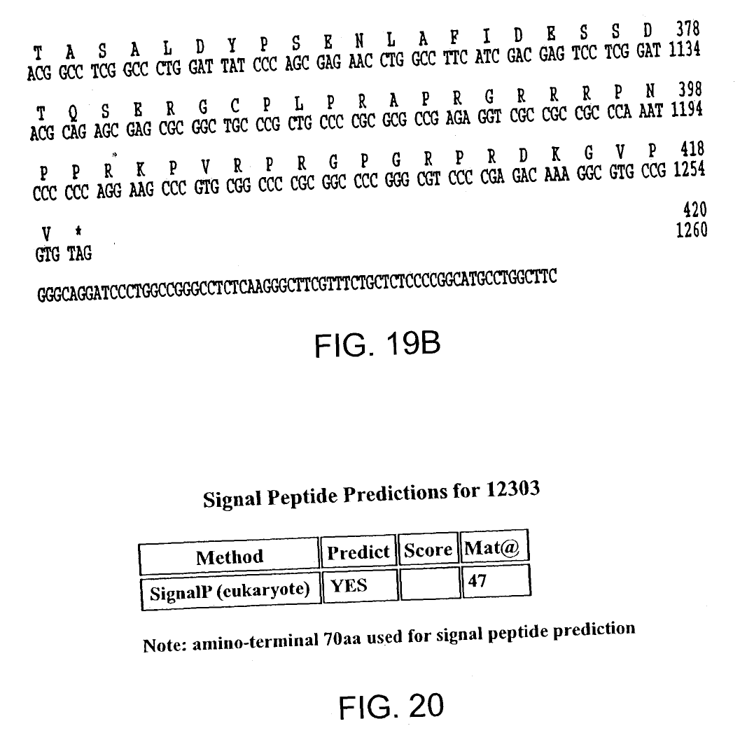

The nucleotide sequence of the isolated human TWIK-8 cDNA and the predicted amino acid sequence of the human TWIK-8 polypeptide are shown in FIG. 19 and in SEQ ID NOs:28 and 29, respectively. A plasmid containing the nucleotide sequence encoding human TWIK-8 was deposited with the American Type Culture Collection (ATCC), 10801 University Boulevard, Manassas, Va. 20110-2209, on and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The human TWIK-8 gene, which is approximately 1408 nucleotides in length, encodes a protein having a molecular weight of approximately 46.1 kD and which is approximately 419 amino acid residues in length.

Various aspects of the invention are described in further detail in the following subsections:

I. Isolated Nucleic Acid Molecules

One aspect of the invention pertains to isolated nucleic acid molecules that encode TWIK-8 proteins or biologically active portions thereof, as well as nucleic acid fragments sufficient for use as hybridization probes to identify TWIK-8-encoding nucleic acid molecules (e.g., TWIK-8 mRNA) and fragments for use as PCR primers for the amplification or mutation of TWIK-8 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TWIK-8 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:28 or 30, or the nucleotide sequence of the DNA insert of the plasmid deposited with ATCC as Accession Number ______, or a portion thereof, can be isolated using standard molecular biology techniques and the sequence information provided herein. Using all or portion of the nucleic acid sequence of SEQ ID NO:28 or 30, or the nucleotide sequence of the DNA insert of the plasmid deposited with ATCC as Accession Number ______, as a hybridization probe, TWIK-8 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:28 or 30, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:28 or 30, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to TWIK-8 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:28. The sequence of SEQ ID NO:28 corresponds to the human TWIK-8 cDNA. This cDNA comprises sequences encoding the human TWIK-8 protein (i.e., “the coding region”, from nucleotides 84-1343), as well as 5′ untranslated sequences (nucleotides 1-83) and 3′ untranslated sequences (nucleotides 1344-1408). Alternatively, the nucleic acid molecule can comprise only the coding region of SEQ ID NO:28 (e.g., nucleotides 84-1343, corresponding to SEQ ID NO:30).

In another preferred embodiment, an isolated nucleic acid molecule of the invention comprises a nucleic acid molecule which is a complement of the nucleotide sequence shown in SEQ ID NO:28 or 30, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:28 or 30, or the nucleotide sequence of the DNA insert of the plasmid deposited with ATCC as Accession Number ______, is one which is sufficiently complementary to the nucleotide sequence shown in SEQ ID NO:28 or 30, or the nucleotide sequence of the DNA insert of the plasmid deposited with ATCC as Accession Number ______, such that it can hybridize to the nucleotide sequence shown in SEQ ID NO:28 or 30, or the nucleotide sequence of the DNA insert of the plasmid deposited with ATCC as Accession Number ______, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 91%, 92%, 93%, 94%, 95%, 96%, 97%, 98%, 99% or more identical to the entire length of the nucleotide sequence shown in SEQ ID NO:28 or 30, or the entire length of the nucleotide sequence of the DNA insert of the plasmid deposited with ATCC as Accession Number ______, or a portion of any of these nucleotide sequences.

Moreover, the nucleic acid molecule of the invention can comprise only a portion of the nucleic acid sequence of SEQ ID NO:28 or 30, or the nucleotide sequence of the DNA insert of the plasmid deposited with ATCC as Accession Number ______, for example, a fragment which can be used as a probe or primer or a fragment encoding a portion of a TWIK-8 protein, e.g., a biologically active portion of a TWIK-8 protein. The nucleotide sequence determined from the cloning of the TWIK-8 gene allows for the generation of probes and primers designed for use in identifying and/or cloning other TWIK-8 family members, as well as TWIK-8 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95, 100, 150, or 200 or more consecutive nucleotides of a sense sequence of SEQ ID NO:28 or 30, or the nucleotide sequence of the DNA insert of the plasmid deposited with ATCC as Accession Number ______, of an anti-sense sequence of SEQ ID NO:28 or 30, or the nucleotide sequence of the DNA insert of the plasmid deposited with ATCC as Accession Number ______, or of a naturally occurring allelic variant or mutant of SEQ ID NO:28 or 30, or the nucleotide sequence of the DNA insert of the plasmid deposited with ATCC as Accession Number ______. In one embodiment, a nucleic acid molecule of the present invention comprises a nucleotide sequence which is greater than 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500 or more nucleotides in length and hybridizes under stringent hybridization conditions to a nucleic acid molecule of SEQ ID NO:28 or 30, or the nucleotide sequence of the DNA insert of the plasmid deposited with ATCC as Accession Number ______.

Probes based on the TWIK-8 nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a TWIK-8 protein, such as by measuring a level of a TWIK-8-encoding nucleic acid in a sample of cells from a subject e.g., detecting TWIK-8 mRNA levels or determining whether a genomic TWIK-8 gene has been mutated or deleted.

A nucleic acid fragment encoding a “biologically active portion of a TWIK-8 protein” can be prepared by isolating a portion of the nucleotide sequence of SEQ ID NO:28 or 30, or the nucleotide sequence of the DNA insert of the plasmid deposited with ATCC as Accession Number ______, which encodes a polypeptide having a TWIK-8 biological activity (the biological activities of the TWIK-8 proteins are described herein), expressing the encoded portion of the TWIK-8 protein (e.g., by recombinant expression in vitro) and assessing the activity of the encoded portion of the TWIK-8 protein.

The invention further encompasses nucleic acid molecules that differ from the nucleotide sequence shown in SEQ ID NO:28 or 30, or the nucleotide sequence of the DNA insert of the plasmid deposited with ATCC as Accession Number ______, due to degeneracy of the genetic code and thus encode the same TWIK-8 proteins as those encoded by the nucleotide sequence shown in SEQ ID NO:28 or 30, or the nucleotide sequence of the DNA insert of the plasmid deposited with ATCC as Accession Number ______. In another embodiment, an isolated nucleic acid molecule of the invention has a nucleotide sequence encoding a protein having an amino acid sequence shown in SEQ ID NO:29.

In addition to the TWIK-8 nucleotide sequences shown in SEQ ID NO:28 or 30, or the nucleotide sequence of the DNA insert of the plasmid deposited with ATCC as Accession Number ______, it will be appreciated by those skilled in the art that DNA sequence polymorphisms that lead to changes in the amino acid sequences of the TWIK-8 proteins may exist within a population (e.g., the human population). Such genetic polymorphism in the TWIK-8 genes may exist among individuals within a population due to natural allelic variation. As used herein, the terms “gene” and “recombinant gene” refer to nucleic acid molecules which include an open reading frame encoding a TWIK-8 protein, preferably a mammalian TWIK-8 protein, and can further include non-coding regulatory sequences, and introns.

Allelic variants of human TWIK-8 include both functional and non-functional TWIK-8 proteins. Functional allelic variants are naturally occurring amino acid sequence variants of the human TWIK-8 protein that maintain the ability to bind a TWIK-8 ligand or substrate and/or modulate pain signaling mechanisms, membrane excitability or neurotransmitter release. Functional allelic variants will typically contain only conservative substitution of one or more amino acids of SEQ ID NO:29, or substitution, deletion or insertion of non-critical residues in non-critical regions of the protein.

Non-functional allelic variants are naturally occurring amino acid sequence variants of the human TWIK-8 protein that do not have the ability to either bind a TWIK-8 ligand and/or modulate any of the TWIK-8 activities described herein. Non-functional allelic variants will typically contain a non-conservative substitution, a deletion, or insertion or premature truncation of the amino acid sequence of SEQ ID NO:29, or a substitution, insertion or deletion in critical residues or critical regions.

The present invention further provides non-human orthologues of the human TWIK-8 protein. Orthologues of the human TWIK-8 protein are proteins that are isolated from non-human organisms and possess the same TWIK-8 ligand binding and/or modulation of membrane excitability activities of the human TWIK-8 protein. Orthologues of the human TWIK-8 protein can readily be identified as comprising an amino acid sequence that is substantially identical to SEQ ID NO:29.

Moreover, nucleic acid molecules encoding other TWIK-8 family members and, thus, which have a nucleotide sequence which differs from the TWIK-8 sequences of SEQ ID NO:28 or 30, or the nucleotide sequence of the DNA insert of the plasmid deposited with ATCC as Accession Number ______ are intended to be within the scope of the invention. For example, another TWIK-8 cDNA can be identified based on the nucleotide sequence of human TWIK-8. Moreover, nucleic acid molecules encoding TWIK-8 proteins from different species, and which, thus, have a nucleotide sequence which differs from the TWIK-8 sequences of SEQ ID NO:28 or 30, or the nucleotide sequence of the DNA insert of the plasmid deposited with ATCC as Accession Number ______ are intended to be within the scope of the invention. For example, a mouse TWIK-8 cDNA can be identified based on the nucleotide sequence of a human TWIK-8.

Nucleic acid molecules corresponding to natural allelic variants and homologues of the TWIK-8 cDNAs of the invention can be isolated based on their homology to the TWIK-8 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the TWIK-8 cDNAs of the invention can further be isolated by mapping to the same chromosome or locus as the TWIK-8 gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:28 or 30, or the nucleotide sequence of the DNA insert of the plasmid deposited with ATCC as Accession Number ______. In other embodiment, the nucleic acid is at least 950-1000, 1000-1100, 1100-1200, 1200-1300, 1300-1400, 1400-1500 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

Preferably, an isolated nucleic acid molecule of the invention that hybridizes under stringent conditions to the sequence of SEQ ID NO:28 or 30 and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the TWIK-8 sequences that may exist in the population, the skilled artisan will further appreciate that changes can be introduced by mutation into the nucleotide sequences of SEQ ID NO:28 or 30, or the nucleotide sequence of the DNA insert of the plasmid deposited with ATCC as Accession Number ______, thereby leading to changes in the amino acid sequence of the encoded TWIK-8 proteins, without altering the functional ability of the TWIK-8 proteins. For example, nucleotide substitutions leading to amino acid substitutions at “non-essential” amino acid residues can be made in the sequence of SEQ ID NO:28 or 30, or the nucleotide sequence of the DNA insert of the plasmid deposited with ATCC as Accession Number ______. A “non-essential” amino acid residue is a residue that can be altered from the wild-type sequence of TWIK-8 (e.g., the sequence of SEQ ID NO:29) without altering the biological activity, whereas an “essential” amino acid residue is required for biological activity. For example, amino acid residues that are conserved among the TWIK-8 proteins of the present invention, e.g., those present in a transmembrane domain, are predicted to be particularly unamenable to alteration. Furthermore, additional amino acid residues that are conserved between the TWIK-8 proteins of the present invention and other members of the TWIK family are not likely to be amenable to alteration.

Accordingly, another aspect of the invention pertains to nucleic acid molecules encoding TWIK-8 proteins that contain changes in amino acid residues that are not essential for activity. Such TWIK-8 proteins differ in amino acid sequence from SEQ ID NO:29, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 91%, 92%, 93%, 94%, 95%, 96%, 97%, 98%, 99% or more identical to SEQ ID NO:29.

An isolated nucleic acid molecule encoding a TWIK-8 protein identical to the protein of SEQ ID NO:29, can be created by introducing one or more nucleotide substitutions, additions or deletions into the nucleotide sequence of SEQ ID NO:28 or 30, or the nucleotide sequence of the DNA insert of the plasmid deposited with ATCC as Accession Number ______, such that one or more amino acid substitutions, additions or deletions are introduced into the encoded protein. Mutations can be introduced into SEQ ID NO:28 or 30, or the nucleotide sequence of the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a TWIK-8 protein is preferably replaced with another amino acid residue from the same side chain family. Alternatively, in another embodiment, mutations can be introduced randomly along all or part of a TWIK-8 coding sequence, such as by saturation mutagenesis, and the resultant mutants can be screened for TWIK-8 biological activity to identify mutants that retain activity. Following mutagenesis of SEQ ID NO:28 or 30, or the nucleotide sequence of the DNA insert of the plasmid deposited with ATCC as Accession Number ______, the encoded protein can be expressed recombinantly and the activity of the protein can be determined.

In a preferred embodiment, a mutant TWIK-8 protein can be assayed for the ability to (1) interact with a non-TWIK protein molecule; (2) activate a TWIK-dependent signal transduction pathway; (3) modulate the release of neurotransmitters; (4) modulate membrane excitability; (5) influence the resting potential of membranes, wave forms and frequencies of action potentials, and thresholds of excitation, (6) modulate processes which underlie learning and memory, such as integration of sub-threshold synaptic responses and the conductance of back-propagating action potentials, and (7) mediate nociception.

In addition to the nucleic acid molecules encoding TWIK-8 proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TWIK-8 coding strand, or to only a portion thereof. In one embodiment, an antisense nucleic acid molecule is antisense to a “coding region” of the coding strand of a nucleotide sequence encoding TWIK-8. The term “coding region” refers to the region of the nucleotide sequence comprising codons which are translated into amino acid residues (e.g., the coding region of human TWIK-8 corresponds to SEQ ID NO:30). In another embodiment, the antisense nucleic acid molecule is antisense to a “noncoding region” of the coding strand of a nucleotide sequence encoding TWIK-8. The term “noncoding region” refers to 5′ and 3′ sequences which flank the coding region that are not translated into amino acids (i.e., also referred to as 5′ and 3′ untranslated regions).

Given the coding strand sequences encoding TWIK-8 disclosed herein (e.g., SEQ ID NO:30), antisense nucleic acids of the invention can be designed according to the rules of Watson and Crick base pairing. The antisense nucleic acid molecule can be complementary to the entire coding region of TWIK-8 mRNA, but more preferably is an oligonucleotide which is antisense to only a portion of the coding or noncoding region of TWIK-8 mRNA. For example, the antisense oligonucleotide can be complementary to the region surrounding the translation start site of TWIK-8 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a TWIK-8 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave TWIK-8 mRNA transcripts to thereby inhibit translation of TWIK-8 mRNA. A ribozyme having specificity for a TWIK-8-encoding nucleic acid can be designed based upon the nucleotide sequence of a TWIK-8 cDNA disclosed herein (i.e., SEQ ID NO:28 or 30, or the nucleotide sequence of the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in a TWIK-8-encoding mRNA. See, e.g., Cech et al. U.S. Pat. No. 4,987,071; and Cech et al. U.S. Pat. No. 5,116,742. Alternatively, TWIK-8 mRNA can be used to select a catalytic RNA having a specific ribonuclease activity from a pool of RNA molecules. See, e.g., Bartel, D. and Szostak, J. W. (1993) Science 261:1411-1418.

Alternatively, TWIK-8 gene expression can be inhibited by targeting nucleotide sequences complementary to the regulatory region of the TWIK-8 (e.g., the TWIK-8 promoter and/or enhancers; e.g., nucleotides 1-83 of SEQ ID NO:28) to form triple helical structures that prevent transcription of the TWIK-8 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the TWIK-8 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of TWIK-8 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of TWIK-8 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of TWIK-8 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of TWIK-8 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous TWIK-8 gene within a cell line or microorganism may be modified by inserting a heterologous DNA regulatory element into the genome of a stable cell line or cloned microorganism such that the inserted regulatory element is operatively linked with the endogenous TWIK-8 gene. For example, an endogenous TWIK-8 gene which is normally “transcriptionally silent”, i.e., a TWIK-8 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous TWIK-8 gene may be activated by insertion of a promiscuous regulatory element that works across cell types.

A heterologous regulatory element may be inserted into a stable cell line or cloned microorganism, such that it is operatively linked with an endogenous TWIK-8 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated TWIK-8 Proteins and Anti-TWIK-8 Antibodies

One aspect of the invention pertains to isolated TWIK-8 proteins, and biologically active portions thereof, as well as polypeptide fragments suitable for use as immunogens to raise anti-TWIK-8 antibodies. In one embodiment, native TWIK-8 proteins can be isolated from cells or tissue sources by an appropriate purification scheme using standard protein purification techniques. In another embodiment, TWIK-8 proteins are produced by recombinant DNA techniques. Alternative to recombinant expression, a TWIK-8 protein or polypeptide can be synthesized chemically using standard peptide synthesis techniques.

An “isolated” or “purified” protein or biologically active portion thereof is substantially free of cellular material or other contaminating proteins from the cell or tissue source from which the TWIK-8 protein is derived, or substantially free from chemical precursors or other chemicals when chemically synthesized. The language “substantially free of cellular material” includes preparations of TWIK-8 protein in which the protein is separated from cellular components of the cells from which it is isolated or recombinantly produced. In one embodiment, the language “substantially free of cellular material” includes preparations of TWIK-8 protein having less than about 30% (by dry weight) of non-TWIK-8 protein (also referred to herein as a “contaminating protein”), more preferably less than about 20% of non-TWIK-8 protein, still more preferably less than about 10% of non-TWIK-8 protein, and most preferably less than about 5% non-TWIK-8 protein. When the TWIK-8 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of TWIK-8 protein in which the protein is separated from chemical precursors or other chemicals which are involved in the synthesis of the protein. In one embodiment, the language “substantially free of chemical precursors or other chemicals” includes preparations of TWIK-8 protein having less than about 30% (by dry weight) of chemical precursors or non-TWIK-8 chemicals, more preferably less than about 20% chemical precursors or non-TWIK-8 chemicals, still more preferably less than about 10% chemical precursors or non-TWIK-8 chemicals, and most preferably less than about 5% chemical precursors or non-TWIK-8 chemicals.

As used herein, a “biologically active portion” of a TWIK-8 protein includes a fragment of a TWIK-8 protein which participates in an interaction between a TWIK-8 molecule and a non-TWIK-8 molecule. Biologically active portions of a TWIK-8 protein include peptides comprising amino acid sequences sufficiently identical to or derived from the amino acid sequence of the TWIK-8 protein, e.g., the amino acid sequence shown in SEQ ID NO:29, which include less amino acids than the full length TWIK-8 proteins, and exhibit at least one activity of a TWIK-8 protein. Typically, biologically active portions comprise a domain or motif with at least one activity of the TWIK-8 protein, e.g., modulating membrane excitability. A biologically active portion of a TWIK-8 protein can be a polypeptide which is, for example, 25, 30, 35, 40, 45, 50, 75, 100, 125, 150, 175, 200 or more amino acids in length. Biologically active portions of a TWIK-8 protein can be used as targets for developing agents which modulate a TWIK-8 mediated activity, e.g., modulation of membrane excitability.

In one embodiment, a biologically active portion of a TWIK-8 protein comprises at least one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain. It is to be understood that a preferred biologically active portion of a TWIK-8 protein of the present invention may contain at least one transmembrane domain and one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain. Moreover, other biologically active portions, in which other regions of the protein are deleted, can be prepared by recombinant techniques and evaluated for one or more of the functional activities of a native TWIK-8 protein.

In a preferred embodiment, the TWIK-8 protein has an amino acid sequence shown in SEQ ID NO:29. In other embodiments, the TWIK-8 protein is substantially identical to SEQ ID NO:29, and retains the functional activity of the protein of SEQ ID NO:29, yet differs in amino acid sequence due to natural allelic variation or mutagenesis, as described in detail in subsection I above. Accordingly, in another embodiment, the TWIK-8 protein is a protein which comprises an amino acid sequence at least about 50%, 55%, 60%, 65%, 70%, 75%, 80%, 85%, 90%, 91%, 92%, 93%, 94%, 95%, 96%, 97%, 98%, 99% or more identical to SEQ ID NO:29.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the TWIK-8 amino acid sequence of SEQ ID NO:29 having 419 amino acid residues, at least 120, preferably at least 160, more preferably at least 201, even more preferably at least 241, and even more preferably at least 281 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to TWIK-8 nucleic acid molecules of the invention. BLAST protein searches can be performed with the XBLAST program, score=100, wordlength=3 to obtain amino acid sequences homologous to TWIK-8 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides TWIK-8 chimeric or fusion proteins. As used herein, a TWIK-8 “chimeric protein” or “fusion protein” comprises a TWIK-8 polypeptide operatively linked to a non-TWIK-8 polypeptide. An “TWIK-8 polypeptide” refers to a polypeptide having an amino acid sequence corresponding to TWIK-8, whereas a “non-TWIK-8 polypeptide” refers to a polypeptide having an amino acid sequence corresponding to a protein which is not substantially homologous to the TWIK-8 protein, e.g., a protein which is different from the TWIK-8 protein and which is derived from the same or a different organism. Within a TWIK-8 fusion protein the TWIK-8 polypeptide can correspond to all or a portion of a TWIK-8 protein. In a preferred embodiment, a TWIK-8 fusion protein comprises at least one biologically active portion of a TWIK-8 protein. In another preferred embodiment, a TWIK-8 fusion protein comprises at least two biologically active portions of a TWIK-8 protein. Within the fusion protein, the term “operatively linked” is intended to indicate that the TWIK-8 polypeptide and the non-TWIK-8 polypeptide are fused in-frame to each other. The non-TWIK-8 polypeptide can be fused to the N-terminus or C-terminus of the TWIK-8 polypeptide.

For example, in one embodiment, the fusion protein is a GST-TWIK-8 fusion protein in which the TWIK-8 sequences are fused to the C-terminus of the GST sequences. Such fusion proteins can facilitate the purification of recombinant TWIK-8.

In another embodiment, the fusion protein is a TWIK-8 protein containing a heterologous signal sequence at its N-terminus. In certain host cells (e.g., mammalian host cells), expression and/or secretion of TWIK-8 can be increased through use of a heterologous signal sequence.

The TWIK-8 fusion proteins of the invention can be incorporated into pharmaceutical compositions and administered to a subject in vivo. The TWIK-8 fusion proteins can be used to affect the bioavailability of a TWIK-8 substrate. Use of TWIK-8 fusion proteins may be useful therapeutically for the treatment of disorders caused by, for example, (i) aberrant modification or mutation of a gene encoding a TWIK-8 protein; (ii) mis-regulation of the TWIK-8 gene; and (iii) aberrant post-translational modification of a TWIK-8 protein.

Moreover, the TWIK-8-fusion proteins of the invention can be used as immunogens to produce anti-TWIK-8 antibodies in a subject, to purify TWIK-8 ligands and in screening assays to identify molecules which inhibit the interaction of TWIK-8 with a TWIK-8 substrate.

Preferably, a TWIK-8 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A TWIK-8-encoding nucleic acid can be cloned into such an expression vector such that the fusion moiety is linked in-frame to the TWIK-8 protein.

The present invention also pertains to variants of the TWIK-8 proteins which function as either TWIK-8 agonists (mimetics) or as TWIK-8 antagonists. Variants of the TWIK-8 proteins can be generated by mutagenesis, e.g., discrete point mutation or truncation of a TWIK-8 protein. An agonist of the TWIK-8 proteins can retain substantially the same, or a subset, of the biological activities of the naturally occurring form of a TWIK-8 protein. An antagonist of a TWIK-8 protein can inhibit one or more of the activities of the naturally occurring form of the TWIK-8 protein by, for example, competitively modulating a TWIK-8-mediated activity of a TWIK-8 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the TWIK-8 protein.

In one embodiment, variants of a TWIK-8 protein which function as either TWIK-8 agonists (mimetics) or as TWIK-8 antagonists can be identified by screening combinatorial libraries of mutants, e.g., truncation mutants, of a TWIK-8 protein for TWIK-8 protein agonist or antagonist activity. In one embodiment, a variegated library of TWIK-8 variants is generated by combinatorial mutagenesis at the nucleic acid level and is encoded by a variegated gene library. A variegated library of TWIK-8 variants can be produced by, for example, enzymatically ligating a mixture of synthetic oligonucleotides into gene sequences such that a degenerate set of potential TWIK-8 sequences is expressible as individual polypeptides, or alternatively, as a set of larger fusion proteins (e.g., for phage display) containing the set of TWIK-8 sequences therein. There are a variety of methods which can be used to produce libraries of potential TWIK-8 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TWIK-8 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a TWIK-8 protein coding sequence can be used to generate a variegated population of TWIK-8 fragments for screening and subsequent selection of variants of a TWIK-8 protein. In one embodiment, a library of coding sequence fragments can be generated by treating a double stranded PCR fragment of a TWIK-8 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the TWIK-8 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TWIK-8 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify TWIK-8 variants (Arkin and Youvan (1992) Proc. Natl. Acad. Sci. USA 89:7811-7815; Delagrave et al. (1993) Protein Engineering 6(3): 327-331).

In one embodiment, cell based assays can be exploited to analyze a variegated TWIK-8 library. For example, a library of expression vectors can be transfected into a cell line, e.g., a neuronal cell line, which ordinarily responds to a TWIK-8 ligand in a particular TWIK-8 ligand-dependent manner. The transfected cells are then contacted with a TWIK-8 ligand and the effect of expression of the mutant on, e.g., membrane excitability of TWIK-8 can be detected. Plasmid DNA can then be recovered from the cells which score for inhibition, or alternatively, potentiation of signaling by the TWIK-8 ligand, and the individual clones further characterized.

An isolated TWIK-8 protein, or a portion or fragment thereof, can be used as an immunogen to generate antibodies that bind TWIK-8 using standard techniques for polyclonal and monoclonal antibody preparation. A full-length TWIK-8 protein can be used or, alternatively, the invention provides antigenic peptide fragments of TWIK-8 for use as immunogens. The antigenic peptide of TWIK-8 comprises at least 8 amino acid residues of the amino acid sequence shown in SEQ ID NO:29 and encompasses an epitope of TWIK-8 such that an antibody raised against the peptide forms a specific immune complex with TWIK-8. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of TWIK-8 that are located on the surface of the protein, e.g., hydrophilic regions, as well as regions with high antigenicity.

A TWIK-8 immunogen typically is used to prepare antibodies by immunizing a suitable subject, (e.g., rabbit, goat, mouse or other mammal) with the immunogen. An appropriate immunogenic preparation can contain, for example, recombinantly expressed TWIK-8 protein or a chemically synthesized TWIK-8 polypeptide. The preparation can further include an adjuvant, such as Freund's complete or incomplete adjuvant, or similar immunostimulatory agent. Immunization of a suitable subject with an immunogenic TWIK-8 preparation induces a polyclonal anti-TWIK-8 antibody response.

Accordingly, another aspect of the invention pertains to anti-TWIK-8 antibodies. The term “antibody” as used herein refers to immunoglobulin molecules and immunologically active portions of immunoglobulin molecules, i.e., molecules that contain an antigen binding site which specifically binds (immunoreacts with) an antigen, such as TWIK-8. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind TWIK-8. The term “monoclonal antibody” or “monoclonal antibody composition”, as used herein, refers to a population of antibody molecules that contain only one species of an antigen binding site capable of immunoreacting with a particular epitope of TWIK-8. A monoclonal antibody composition thus typically displays a single binding affinity for a particular TWIK-8 protein with which it immunoreacts.

Polyclonal anti-TWIK-8 antibodies can be prepared as described above by immunizing a suitable subject with a TWIK-8 immunogen. The anti-TWIK-8 antibody titer in the immunized subject can be monitored over time by standard techniques, such as with an enzyme linked immunosorbent assay (ELISA) using immobilized TWIK-8. If desired, the antibody molecules directed against TWIK-8 can be isolated from the mammal (e.g., from the blood) and further purified by well known techniques, such as protein A chromatography to obtain the IgG fraction. At an appropriate time after immunization, e.g., when the anti-TWIK-8 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a TWIK-8 immunogen as described above, and the culture supernatants of the resulting hybridoma cells are screened to identify a hybridoma producing a monoclonal antibody that binds TWIK-8.

Any of the many well known protocols used for fusing lymphocytes and immortalized cell lines can be applied for the purpose of generating an anti-TWIK-8 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind TWIK-8, e.g., using a standard ELISA assay.

Alternative to preparing monoclonal antibody-secreting hybridomas, a monoclonal anti-TWIK-8 antibody can be identified and isolated by screening a recombinant combinatorial immunoglobulin library (e.g., an antibody phage display library) with TWIK-8 to thereby isolate immunoglobulin library members that bind TWIK-8. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-TWIK-8 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-TWIK-8 antibody (e.g., monoclonal antibody) can be used to isolate TWIK-8 by standard techniques, such as affinity chromatography or immunoprecipitation. An anti-TWIK-8 antibody can facilitate the purification of natural TWIK-8 from cells and of recombinantly produced TWIK-8 expressed in host cells. Moreover, an anti-TWIK-8 antibody can be used to detect TWIK-8 protein (e.g., in a cellular lysate or cell supernatant) in order to evaluate the abundance and pattern of expression of the TWIK-8 protein. Anti-TWIK-8 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a TWIK-8 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., TWIK-8 proteins, mutant forms of TWIK-8 proteins, fusion proteins, and the like).

The recombinant expression vectors of the invention can be designed for expression of TWIK-8 proteins in prokaryotic or eukaryotic cells. For example, TWIK-8 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in TWIK-8 activity assays, (e.g., direct assays or competitive assays described in detail below), or to generate antibodies specific for TWIK-8 proteins, for example. In a preferred embodiment, a TWIK-8 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the TWIK-8 expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corporation, San Diego, Calif.).

Alternatively, TWIK-8 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to TWIK-8 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a TWIK-8 nucleic acid molecule of the invention is introduced, e.g, a TWIK-8 nucleic acid molecule within a recombinant expression vector or a TWIK-8 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a TWIK-8 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a TWIK-8 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a TWIK-8 protein. Accordingly, the invention further provides methods for producing a TWIK-8 protein using the host cells of the invention. In one embodiment, the method comprises culturing the host cell of the invention (into which a recombinant expression vector encoding a TWIK-8 protein has been introduced) in a suitable medium such that a TWIK-8 protein is produced. In another embodiment, the method further comprises isolating a TWIK-8 protein from the medium or the host cell.

The host cells of the invention can also be used to produce non-human transgenic animals. For example, in one embodiment, a host cell of the invention is a fertilized oocyte or an embryonic stem cell into which TWIK-8-coding sequences have been introduced. Such host cells can then be used to create non-human transgenic animals in which exogenous TWIK-8 sequences have been introduced into their genome or homologous recombinant animals in which endogenous TWIK-8 sequences have been altered. Such animals are useful for studying the function and/or activity of a TWIK-8 and for identifying and/or evaluating modulators of TWIK-8 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous TWIK-8 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a TWIK-8-encoding nucleic acid into the male pronuclei of a fertilized oocyte, e.g., by microinjection, retroviral infection, and allowing the oocyte to develop in a pseudopregnant female foster animal. The TWIK-8 cDNA sequence of SEQ ID NO:28 can be introduced as a transgene into the genome of a non-human animal. Alternatively, a nonhuman homologue of a human TWIK-8 gene, such as a mouse or rat TWIK-8 gene, can be used as a transgene. Alternatively, a TWIK-8 gene homologue, such as another TWIK-8 family member, can be isolated based on hybridization to the TWIK-8 cDNA sequences of SEQ ID NO:28 or 30, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to a TWIK-8 transgene to direct expression of a TWIK-8 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagneret al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a TWIK-8 transgene in its genome and/or expression of TWIK-8 mRNA in tissues or cells of the animals. A transgenic founder animal can then be used to breed additional animals carrying the transgene. Moreover, transgenic animals carrying a transgene encoding a TWIK-8 protein can further be bred to other transgenic animals carrying other transgenes.

To create a homologous recombinant animal, a vector is prepared which contains at least a portion of a TWIK-8 gene into which a deletion, addition or substitution has been introduced to thereby alter, e.g., functionally disrupt, the TWIK-8 gene. The TWIK-8 gene can be a human gene (e.g., the cDNA of SEQ ID NO:30), but more preferably, is a non-human homologue of a human TWIK-8 gene (e.g., a cDNA isolated by stringent hybridization with the nucleotide sequence of SEQ ID NO:28). For example, a mouse TWIK-8 gene can be used to construct a homologous recombination nucleic acid molecule, e.g., a vector, suitable for altering an endogenous TWIK-8 gene in the mouse genome. In a preferred embodiment, the homologous recombination nucleic acid molecule is designed such that, upon homologous recombination, the endogenous TWIK-8 gene is functionally disrupted (i.e., no longer encodes a functional protein; also referred to as a “knock out” vector). Alternatively, the homologous recombination nucleic acid molecule can be designed such that, upon homologous recombination, the endogenous TWIK-8 gene is mutated or otherwise altered but still encodes functional protein (e.g., the upstream regulatory region can be altered to thereby alter the expression of the endogenous TWIK-8 protein). In the homologous recombination nucleic acid molecule, the altered portion of the TWIK-8 gene is flanked at its 5′ and 3′ ends by additional nucleic acid sequence of the TWIK-8 gene to allow for homologous recombination to occur between the exogenous TWIK-8 gene carried by the homologous recombination nucleic acid molecule and an endogenous TWIK-8 gene in a cell, e.g., an embryonic stem cell. The additional flanking TWIK-8 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced TWIK-8 gene has homologously recombined with the endogenous TWIK-8 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells. A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The TWIK-8 nucleic acid molecules, fragments of TWIK-8 proteins, and anti-TWIK-8 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a TWIK-8 protein or an anti-TWIK-8 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a TWIK-8 protein of the invention has one or more of the following activities: (1) interacting with a non-TWIK protein molecule; (2) activating a TWIK-dependent signal transduction pathway; (3) modulating the release of neurotransmitters; (4) modulating membrane excitability; (5) influencing the resting potential of membranes, wave forms and frequencies of action potentials, and thresholds of excitation, (6) modulating processes which underlie learning and memory, such as integration of sub-threshold synaptic responses and the conductance of back-propagating action potentials, and (7) mediating nociception.

The isolated nucleic acid molecules of the invention can be used, for example, to express TWIK-8 protein (e.g., via a recombinant expression vector in a host cell in gene therapy applications), to detect TWIK-8 mRNA (e.g., in a biological sample) or a genetic alteration in a TWIK-8 gene, and to modulate TWIK-8 activity, as described further below. The TWIK-8 proteins can be used to treat disorders characterized by insufficient or excessive production of a TWIK-8 substrate or production of TWIK-8 inhibitors. In addition, the TWIK-8 proteins can be used to screen for naturally occurring TWIK-8 substrates, to screen for drugs or compounds which modulate TWIK-8 activity, as well as to treat disorders characterized by insufficient or excessive production of TWIK-8 protein or production of TWIK-8 protein forms which have decreased, aberrant or unwanted activity compared to TWIK-8 wild type protein, e.g., potassium channel associated disorders. Such disorders include CNS disorders such as cognitive and neurodegenerative disorders (e.g., Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, AIDS related dementia, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, and bipolar affective disorder (e.g., severe bipolar affective (mood) disorder (BP-1) and bipolar affective neurological disorders (e.g., migraine and obesity)); cardiac disorders (e.g., arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia), muscular disorders (e.g., paralysis, muscle weakness (e.g., ataxia, myotonia, and myokymia), muscular dystrophy (e.g., Duchenne muscular dystrophy or myotonic dystrophy), spinal muscular atrophy, congenital myopathies, central core disease, rod myopathy, central nuclear myopathy, Lambert-Eaton syndrome, denervation, and infantile spinal muscular atrophy (Werdnig-Hoffman disease), pain disorders (e.g., headache (e.g., tension headache or migraine), back pain, cancer pain, arthritis pain, or neurogenic pain), and disorders of cellular growth, differentiation, or migration (e.g., cancer, e.g., carcinoma, sarcoma, or leukemia; tumor angiogenesis and metastasis; skeletal dysplasia; neuronal deficiencies resulting from impaired neural induction and patterning; and hematopoietic and/or myeloproliferative disorders. Moreover, the anti-TWIK-8 antibodies of the invention can be used to detect and isolate TWIK-8 proteins, to regulate the bioavailability of TWIK-8 proteins, and to modulate TWIK-8 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to TWIK-8 proteins, have a stimulatory or inhibitory effect on, for example, TWIK-8 expression or TWIK-8 activity, or have a stimulatory or inhibitory effect on, for example, the expression or activity of TWIK-8 substrate.

In one embodiment, the invention provides assays for screening candidate or test compounds which are substrates of a TWIK-8 protein or polypeptide or biologically active portion thereof. In another embodiment, the invention provides assays for screening candidate or test compounds which bind to or modulate the activity of a TWIK-8 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a TWIK-8 protein or biologically active portion thereof is contacted with a test compound and the ability of the test compound to modulate TWIK-8 activity is determined. Determining the ability of the test compound to modulate TWIK-8 activity can be accomplished by monitoring, for example, the release of a neurotransmitter from a cell which expresses TWIK-8. The cell, for example, can be of mammalian origin, e.g., a neuronal cell, cardiac cell or a skin cell.

The ability of the test compound to modulate TWIK-8 binding to a substrate or to bind to TWIK-8 can also be determined. Determining the ability of the test compound to modulate TWIK-8 binding to a substrate can be accomplished, for example, by coupling the TWIK-8 substrate with a radioisotope or enzymatic label such that binding of the TWIK-8 substrate to TWIK-8 can be determined by detecting the labeled TWIK-8 substrate in a complex. Alternatively, TWIK-8 could be coupled with a radioisotope or enzymatic label to monitor the ability of a test compound to modulate TWIK-8 binding to a TWIK-8 substrate in a complex. Determining the ability of the test compound to bind TWIK-8 can be accomplished, for example, by coupling the compound with a radioisotope or enzymatic label such that binding of the compound to TWIK-8 can be determined by detecting the labeled TWIK-8 compound in a complex. For example, compounds (e.g., TWIK-8 substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a TWIK-8 substrate) to interact with TWIK-8 without the labeling of any of the interactants. For example, a microphysiometer can be used to detect the interaction of a compound with TWIK-8 without the labeling of either the compound or the TWIK-8. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and TWIK-8.

In another embodiment, an assay is a cell-based assay comprising contacting a cell expressing a TWIK-8 target molecule (e.g., a TWIK-8 substrate) with a test compound and determining the ability of the test compound to modulate (e.g., stimulate or inhibit) the activity of the TWIK-8 target molecule. Determining the ability of the test compound to modulate the activity of a TWIK-8 target molecule can be accomplished, for example, by determining the ability of the TWIK-8 protein to bind to or interact with the TWIK-8 target molecule.

Determining the ability of the TWIK-8 protein, or a biologically active fragment thereof, to bind to or interact with a TWIK-8 target molecule can be accomplished by one of the methods described above for determining direct binding. In a preferred embodiment, determining the ability of the TWIK-8 protein to bind to or interact with a TWIK-8 target molecule can be accomplished by determining the activity of the target molecule. For example, the activity of the target molecule can be determined by detecting induction of a cellular response (i.e., changes in intracellular K⁺ levels), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which a TWIK-8 protein or biologically active portion thereof is contacted with a test compound and the ability of the test compound to bind to the TWIK-8 protein or biologically active portion thereof is determined. Preferred biologically active portions of the TWIK-8 proteins to be used in assays of the present invention include fragments which participate in interactions with non-TWIK-8 molecules, e.g., fragments with high surface probability scores. Binding of the test compound to the TWIK-8 protein can be determined either directly or indirectly as described above. In a preferred embodiment, the assay includes contacting the TWIK-8 protein or biologically active portion thereof with a known compound which binds TWIK-8 to form an assay mixture, contacting the assay mixture with a test compound, and determining the ability of the test compound to interact with a TWIK-8 protein, wherein determining the ability of the test compound to interact with a TWIK-8 protein comprises determining the ability of the test compound to preferentially bind to TWIK-8 or biologically active portion thereof as compared to the known compound.

In another embodiment, the assay is a cell-free assay in which a TWIK-8 protein or biologically active portion thereof is contacted with a test compound and the ability of the test compound to modulate (e.g., stimulate or inhibit) the activity of the TWIK-8 protein or biologically active portion thereof is determined. Determining the ability of the test compound to modulate the activity of a TWIK-8 protein can be accomplished, for example, by determining the ability of the TWIK-8 protein to bind to a TWIK-8 target molecule by one of the methods described above for determining direct binding. Determining the ability of the TWIK-8 protein to bind to a TWIK-8 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a TWIK-8 protein can be accomplished by determining the ability of the TWIK-8 protein to further modulate the activity of a downstream effector of a TWIK-8 target molecule. For example, the activity of the effector molecule on an appropriate target can be determined or the binding of the effector to an appropriate target can be determined as previously described.

In yet another embodiment, the cell-free assay involves contacting a TWIK-8 protein or biologically active portion thereof with a known compound which binds the TWIK-8 protein to form an assay mixture, contacting the assay mixture with a test compound, and determining the ability of the test compound to interact with the TWIK-8 protein, wherein determining the ability of the test compound to interact with the TWIK-8 protein comprises determining the ability of the TWIK-8 protein to preferentially bind to or modulate the activity of a TWIK-8 target molecule.

In more than one embodiment of the above assay methods of the present invention, it may be desirable to immobilize either TWIK-8 or its target molecule to facilitate separation of complexed from uncomplexed forms of one or both of the proteins, as well as to accommodate automation of the assay. Binding of a test compound to a TWIK-8 protein, or interaction of a TWIK-8 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/TWIK-8 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or TWIK-8 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of TWIK-8 binding or activity determined using standard techniques.

Other techniques for immobilizing proteins on matrices can also be used in the screening assays of the invention. For example, either a TWIK-8 protein or a TWIK-8 target molecule can be immobilized utilizing conjugation of biotin and streptavidin. Biotinylated TWIK-8 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with TWIK-8 protein or target molecules but which do not interfere with binding of the TWIK-8 protein to its target molecule can be derivatized to the wells of the plate, and unbound target or TWIK-8 protein trapped in the wells by antibody conjugation. Methods for detecting such complexes, in addition to those described above for the GST-immobilized complexes, include immunodetection of complexes using antibodies reactive with the TWIK-8 protein or target molecule, as well as enzyme-linked assays which rely on detecting an enzymatic activity associated with the TWIK-8 protein or target molecule.

In another embodiment, modulators of TWIK-8 expression are identified in a method wherein a cell is contacted with a candidate compound and the expression of TWIK-8 mRNA or protein in the cell is determined. The level of expression of TWIK-8 mRNA or protein in the presence of the candidate compound is compared to the level of expression of TWIK-8 mRNA or protein in the absence of the candidate compound. The candidate compound can then be identified as a modulator of TWIK-8 expression based on this comparison. For example, when expression of TWIK-8 mRNA or protein is greater (statistically significantly greater) in the presence of the candidate compound than in its absence, the candidate compound is identified as a stimulator of TWIK-8 mRNA or protein expression. Alternatively, when expression of TWIK-8 mRNA or protein is less (statistically significantly less) in the presence of the candidate compound than in its absence, the candidate compound is identified as an inhibitor of TWIK-8 mRNA or protein expression. The level of TWIK-8 mRNA or protein expression in the cells can be determined by methods described herein for detecting TWIK-8 mRNA or protein.

In yet another aspect of the invention, the TWIK-8 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with TWIK-8 (“TWIK-8-binding proteins” or “TWIK-8-bp”) and are involved in TWIK-8 activity. Such TWIK-8-binding proteins are also likely to be involved in the propagation of signals by the TWIK-8 proteins or TWIK-8 targets as, for example, downstream elements of a TWIK-8-mediated signaling pathway. Alternatively, such TWIK-8-binding proteins are likely to be TWIK-8 inhibitors.

The two-hybrid system is based on the modular nature of most transcription factors, which consist of separable DNA-binding and activation domains. Briefly, the assay utilizes two different DNA constructs. In one construct, the gene that codes for a TWIK-8 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TWIK-8-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the TWIK-8 protein.

In another aspect, the invention pertains to a combination of two or more of the assays described herein. For example, a modulating agent can be identified using a cell-based or a cell free assay, and the ability of the agent to modulate the activity of a TWIK-8 protein can be confirmed in vivo, e.g., in an animal such as an animal model for pain.

This invention further pertains to novel agents identified by the above-described screening assays. Accordingly, it is within the scope of this invention to further use an agent identified as described herein in an appropriate animal model. For example, an agent identified as described herein (e.g., a TWIK-8 modulating agent, an antisense TWIK-8 nucleic acid molecule, a TWIK-8-specific antibody, or a TWIK-8-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the TWIK-8 nucleotide sequences, described herein, can be used to map the location of the TWIK-8 genes on a chromosome. The mapping of the TWIK-8 sequences to chromosomes is an important first step in correlating these sequences with genes associated with disease.

Briefly, TWIK-8 genes can be mapped to chromosomes by preparing PCR primers (preferably 15-25 bp in length) from the TWIK-8 nucleotide sequences. Computer analysis of the TWIK-8 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the TWIK-8 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the TWIK-8 nucleotide sequences to design oligonucleotide primers, sublocalization can be achieved with panels of fragments from specific chromosomes. Other mapping strategies which can similarly be used to map a TWIK-8 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the TWIK-8 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The TWIK-8 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the TWIK-8 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The TWIK-8 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:28 can comfortably provide positive individual identification with a panel of perhaps 10 to 1,000 primers which each yield a noncoding amplified sequence of 100 bases. If predicted coding sequences, such as those in SEQ ID NO:30 are used, a more appropriate number of primers for positive individual identification would be 500-2,000.

If a panel of reagents from TWIK-8 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of TWIK-8 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:28 are particularly appropriate for this use as greater numbers of polymorphisms occur in the noncoding regions, making it easier to differentiate individuals using this technique. Examples of polynucleotide reagents include the TWIK-8 nucleotide sequences or portions thereof, e.g., fragments derived from the noncoding regions of SEQ ID NO:28 having a length of at least 20 bases, preferably at least 30 bases.

The TWIK-8 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such TWIK-8 probes can be used to identify tissue by species and/or by organ type.

In a similar fashion, these reagents, e.g., TWIK-8 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining TWIK-8 protein and/or nucleic acid expression as well as TWIK-8 activity, in the context of a biological sample (e.g., blood, serum, cells, tissue) to thereby determine whether an individual is afflicted with a disease or disorder, or is at risk of developing a disorder, associated with aberrant or unwanted TWIK-8 expression or activity. The invention also provides for prognostic (or predictive) assays for determining whether an individual is at risk of developing a disorder associated with TWIK-8 protein, nucleic acid expression or activity. For example, mutations in a TWIK-8 gene can be assayed in a biological sample. Such assays can be used for prognostic or predictive purpose to thereby prophylactically treat an individual prior to the onset of a disorder characterized by or associated with TWIK-8 protein, nucleic acid expression or activity.

Another aspect of the invention pertains to monitoring the influence of agents (e.g., drugs, compounds) on the expression or activity of TWIK-8 in clinical trials.

These and other agents are described in further detail in the following sections.

1. Diagnostic Assays

An exemplary method for detecting the presence or absence of TWIK-8 protein or nucleic acid in a biological sample involves obtaining a biological sample from a test subject and contacting the biological sample with a compound or an agent capable of detecting TWIK-8 protein or nucleic acid (e.g., mRNA, or genomic DNA) that encodes TWIK-8 protein such that the presence of TWIK-8 protein or nucleic acid is detected in the biological sample. A preferred agent for detecting TWIK-8 mRNA or genomic DNA is a labeled nucleic acid probe capable of hybridizing to TWIK-8 mRNA or genomic DNA. The nucleic acid probe can be, for example, the TWIK-8 nucleic acid set forth in SEQ ID NO:28 or 30, or the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to TWIK-8 mRNA or genomic DNA. Other suitable probes for use in the diagnostic assays of the invention are described herein.

A preferred agent for detecting TWIK-8 protein is an antibody capable of binding to TWIK-8 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect TWIK-8 mRNA, protein, or genomic DNA in a biological sample in vitro as well as in vivo. For example, in vitro techniques for detection of TWIK-8 mRNA include Northern hybridizations and in situ hybridizations. In vitro techniques for detection of TWIK-8 protein include enzyme linked immunosorbent assays (ELISAs), Western blots, immunoprecipitations and immunofluorescence. In vitro techniques for detection of TWIK-8 genomic DNA include Southern hybridizations. Furthermore, in vivo techniques for detection of TWIK-8 protein include introducing into a subject a labeled anti-TWIK-8 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting TWIK-8 protein, mRNA, or genomic DNA, such that the presence of TWIK-8 protein, mRNA or genomic DNA is detected in the biological sample, and comparing the presence of TWIK-8 protein, mRNA or genomic DNA in the control sample with the presence of TWIK-8 protein, mRNA or genomic DNA in the test sample.

The invention also encompasses kits for detecting the presence of TWIK-8 in a biological sample. For example, the kit can comprise a labeled compound or agent capable of detecting TWIK-8 protein or mRNA in a biological sample; means for determining the amount of TWIK-8 in the sample; and means for comparing the amount of TWIK-8 in the sample with a standard. The compound or agent can be packaged in a suitable container. The kit can further comprise instructions for using the kit to detect TWIK-8 protein or nucleic acid.

2. Prognostic Assays

The diagnostic methods described herein can furthermore be utilized to identify subjects having or at risk of developing a disease or disorder associated with aberrant or unwanted TWIK-8 expression or activity. As used herein, the term “aberrant” includes a TWIK-8 expression or activity which deviates from the wild type TWIK-8 expression or activity. Aberrant expression or activity includes increased or decreased expression or activity, as well as expression or activity which does not follow the wild type developmental pattern of expression or the subcellular pattern of expression. For example, aberrant TWIK-8 expression or activity is intended to include the cases in which a mutation in the TWIK-8 gene causes the TWIK-8 gene to be under-expressed or over-expressed and situations in which such mutations result in a non-functional TWIK-8 protein or a protein which does not function in a wild-type fashion, e.g., a protein which does not interact with a TWIK-8 substrate, e.g., a non-potassium channel subunit or ligand, or one which interacts with a non-TWIK-8 substrate, e.g. a non-potassium channel subunit or ligand. As used herein, the term “unwanted” includes an unwanted phenomenon involved in a biological response such as cellular proliferation. For example, the term unwanted includes a TWIK-8 expression or activity which is undesirable in a subject.

The assays described herein, such as the preceding diagnostic assays or the following assays, can be utilized to identify a subject having or at risk of developing a disorder associated with a misregulation in TWIK-8 protein activity or nucleic acid expression, such as a CNS disorder (e.g., a cognitive or neurodegenerative disorder, or a pain disorder), a muscular disorder, a cellular proliferation, growth, differentiation, or migration disorder, or a cardiovascular disorder. Alternatively, the prognostic assays can be utilized to identify a subject having or at risk for developing a disorder associated with a misregulation in TWIK-8 protein activity or nucleic acid expression, such as a CNS disorder, a cardiovascular disorder, a pain disorder, or a cellular proliferation, growth, differentiation, or migration disorder. Thus, the present invention provides a method for identifying a disease or disorder associated with aberrant or unwanted TWIK-8 expression or activity in which a test sample is obtained from a subject and TWIK-8 protein or nucleic acid (e.g., mRNA or genomic DNA) is detected, wherein the presence of TWIK-8 protein or nucleic acid is diagnostic for a subject having or at risk of developing a disease or disorder associated with aberrant or unwanted TWIK-8 expression or activity. As used herein, a “test sample” refers to a biological sample obtained from a subject of interest. For example, a test sample can be a biological fluid (e.g., cerebrospinal fluid or serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted TWIK-8 expression or activity. For example, such methods can be used to determine whether a subject can be effectively treated with an agent for a CNS disorder, a muscular disorder, a pain disorder, a cardiovascular, or a cellular proliferation, growth, differentiation, or migration disorder. Thus, the present invention provides methods for determining whether a subject can be effectively treated with an agent for a disorder associated with aberrant or unwanted TWIK-8 expression or activity in which a test sample is obtained and TWIK-8 protein or nucleic acid expression or activity is detected (e.g., wherein the abundance of TWIK-8 protein or nucleic acid expression or activity is diagnostic for a subject that can be administered the agent to treat a disorder associated with aberrant or unwanted TWIK-8 expression or activity).

The methods of the invention can also be used to detect genetic alterations in a TWIK-8 gene, thereby determining if a subject with the altered gene is at risk for a disorder characterized by misregulation in TWIK-8 protein activity or nucleic acid expression, such as a CNS disorder, a cardiovascular disorder, a pain disorder, or a disorder of cellular growth, differentiation, or migration. In preferred embodiments, the methods include detecting, in a sample of cells from the subject, the presence or absence of a genetic alteration characterized by at least one of an alteration affecting the integrity of a gene encoding a TWIK-8-protein, or the mis-expression of the TWIK-8 gene. For example, such genetic alterations can be detected by ascertaining the existence of at least one of 1) a deletion of one or more nucleotides from a TWIK-8 gene; 2) an addition of one or more nucleotides to a TWIK-8 gene; 3) a substitution of one or more nucleotides of a TWIK-8 gene, 4) a chromosomal rearrangement of a TWIK-8 gene; 5) an alteration in the level of a messenger RNA transcript of a TWIK-8 gene, 6) aberrant modification of a TWIK-8 gene, such as of the methylation pattern of the genomic DNA, 7) the presence of a non-wild type splicing pattern of a messenger RNA transcript of a TWIK-8 gene, 8) a non-wild type level of a TWIK-8-protein, 9) allelic loss of a TWIK-8 gene, and 10) inappropriate post-translational modification of a TWIK-8-protein. As described herein, there are a large number of assays known in the art which can be used for detecting alterations in a TWIK-8 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the TWIK-8-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a TWIK-8 gene under conditions such that hybridization and amplification of the TWIK-8-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a TWIK-8 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in TWIK-8 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in TWIK-8 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the TWIK-8 gene and detect mutations by comparing the sequence of the sample TWIK-8 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the TWIK-8 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type TWIK-8 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in TWIK-8 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a TWIK-8 sequence, e.g., a wild-type TWIK-8 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in TWIK-8 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control TWIK-8 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele-specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a TWIK-8 gene.

Furthermore, any cell type or tissue in which TWIK-8 is expressed may be utilized in the prognostic assays described herein.

3. Monitoring of Effects During Clinical Trials

Monitoring the influence of agents (e.g., drugs) on the expression or activity of a TWIK-8 protein (e.g., the modulation of pain signaling mechanisms, neurotransmitter release and/or membrane excitability) can be applied not only in basic drug screening, but also in clinical trials. For example, the effectiveness of an agent determined by a screening assay as described herein to increase TWIK-8 gene expression, protein levels, or upregulate TWIK-8 activity, can be monitored in clinical trials of subjects exhibiting decreased TWIK-8 gene expression, protein levels, or downregulated TWIK-8 activity. Alternatively, the effectiveness of an agent determined by a screening assay to decrease TWIK-8 gene expression, protein levels, or downregulate TWIK-8 activity, can be monitored in clinical trials of subjects exhibiting increased TWIK-8 gene expression, protein levels, or upregulated TWIK-8 activity. In such clinical trials, the expression or activity of a TWIK-8 gene, and preferably, other genes that have been implicated in, for example, a TWIK-8-associated disorder can be used as a “read out” or markers of the phenotype of a particular cell.

For example, and not by way of limitation, genes, including TWIK-8, that are modulated in cells by treatment with an agent (e.g., compound, drug or small molecule) which modulates TWIK-8 activity (e.g., identified in a screening assay as described herein) can be identified. Thus, to study the effect of agents on TWIK-8-associated disorders (e.g., disorders characterized by deregulated pain signaling mechanisms, neurotransmitter release and/or membrane excitability), for example, in a clinical trial, cells can be isolated and RNA prepared and analyzed for the levels of expression of TWIK-8 and other genes implicated in the TWIK-8-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of TWIK-8 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a TWIK-8 protein, mRNA, or genomic DNA in the preadministration sample; (iii) obtaining one or more post-administration samples from the subject; (iv) detecting the level of expression or activity of the TWIK-8 protein, mRNA, or genomic DNA in the post-administration samples; (v) comparing the level of expression or activity of the TWIK-8 protein, mRNA, or genomic DNA in the pre-administration sample with the TWIK-8 protein, mRNA, or genomic DNA in the post administration sample or samples; and (vi) altering the administration of the agent to the subject accordingly. For example, increased administration of the agent may be desirable to increase the expression or activity of TWIK-8 to higher levels than detected, i.e., to increase the effectiveness of the agent. Alternatively, decreased administration of the agent may be desirable to decrease expression or activity of TWIK-8 to lower levels than detected, i.e. to decrease the effectiveness of the agent. According to such an embodiment, TWIK-8 expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted TWIK-8 expression or activity, e.g. a potassium channel associated disorder such as a CNS disorder, a cardiovascular disorder, a pain disorder, or a cellular proliferation, growth, differentiation, or migration disorder. “Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of disease or disorder or the predisposition toward a disease or disorder. A therapeutic agent includes, but is not limited to, small molecules, peptides, antibodies, ribozymes and antisense oligonucleotides. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the TWIK-8 molecules of the present invention or TWIK-8 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted TWIK-8 expression or activity, by administering to the subject a TWIK-8 or an agent which modulates TWIK-8 expression or at least one TWIK-8 activity. Subjects at risk for a disease which is caused or contributed to by aberrant or unwanted TWIK-8 expression or activity can be identified by, for example, any or a combination of diagnostic or prognostic assays as described herein. Administration of a prophylactic agent can occur prior to the manifestation of symptoms characteristic of the TWIK-8 aberrancy, such that a disease or disorder is prevented or, alternatively, delayed in its progression. Depending on the type of TWIK-8 aberrancy, for example, a TWIK-8, TWIK-8 agonist or TWIK-8 antagonist agent can be used for treating the subject. The appropriate agent can be determined based on screening assays described herein.

2. Therapeutic Methods

Another aspect of the invention pertains to methods of modulating TWIK-8 expression or activity for therapeutic purposes. Accordingly, in an exemplary embodiment, the modulatory method of the invention involves contacting a cell with a TWIK-8 or agent that modulates one or more of the activities of TWIK-8 protein activity associated with the cell. An agent that modulates TWIK-8 protein activity can be an agent as described herein, such as a nucleic acid or a protein, a naturally-occurring target molecule of a TWIK-8 protein (e.g., a TWIK-8 substrate), a TWIK-8 antibody, a TWIK-8 agonist or antagonist, a peptidomimetic of a TWIK-8 agonist or antagonist, or other small molecule. In one embodiment, the agent stimulates one or more TWIK-8 activities. Examples of such stimulatory agents include active TWIK-8 protein and a nucleic acid molecule encoding TWIK-8 that has been introduced into the cell. In another embodiment, the agent inhibits one or more TWIK-8 activities. Examples of such inhibitory agents include antisense TWIK-8 nucleic acid molecules, anti-TWIK-8 antibodies, and TWIK-8 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a TWIK-8 protein or nucleic acid molecule. In one embodiment, the method involves administering an agent (e.g., an agent identified by a screening assay described herein), or combination of agents that modulates (e.g., upregulates or downregulates) TWIK-8 expression or activity. In another embodiment, the method involves administering a TWIK-8 protein or nucleic acid molecule as therapy to compensate for reduced, aberrant, or unwanted TWIK-8 expression or activity.

Stimulation of TWIK-8 activity is desirable in situations in which TWIK-8 is abnormally downregulated and/or in which increased TWIK-8 activity is likely to have a beneficial effect. Likewise, inhibition of TWIK-8 activity is desirable in situations in which TWIK-8 is abnormally upregulated and/or in which decreased TWIK-8 activity is likely to have a beneficial effect.

3. Pharmacogenomics

The TWIK-8 molecules of the present invention, as well as agents, or modulators which have a stimulatory or inhibitory effect on TWIK-8 activity (e.g., TWIK-8 gene expression) as identified by a screening assay described herein can be administered to individuals to treat (prophylactically or therapeutically) TWIK-8-associated disorders (e.g., pain disorders, cardiovascular disorders, proliferative disorders) associated with aberrant or unwanted TWIK-8 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a TWIK-8 molecule or TWIK-8 modulator as well as tailoring the dosage and/or therapeutic regimen of treatment with a TWIK-8 molecule or TWIK-8 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a TWIK-8 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so-called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a TWIK-8 molecule or TWIK-8 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a TWIK-8 molecule or TWIK-8 modulator, such as a modulator identified by one of the exemplary screening assays described herein.

VI. Electronic Apparatus Readable Media and Arrays

Electronic apparatus readable media comprising TWIK-8 sequence information is also provided. As used herein, “TWIK-8 sequence information” refers to any nucleotide and/or amino acid sequence information particular to the TWIK-8 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said TWIK-8 sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon TWIK-8 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the TWIK-8 sequence information.

A variety of software programs and formats can be used to store the sequence information on the electronic apparatus readable medium. For example, the sequence information can be represented in a word processing text file, formatted in commercially-available software such as WordPerfect and Microsoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon the TWIK-8 sequence information.

By providing TWIK-8 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a TWIK-8-associated disease or disorder or a pre-disposition to a TWIK-8-associated disease or disorder, wherein the method comprises the steps of determining TWIK-8 sequence information associated with the subject and based on the TWIK-8 sequence information, determining whether the subject has a TWIK-8-associated disease or disorder or a pre-disposition to a TWIK-8-associated disease or disorder and/or recommending a particular treatment for the disease, disorder or pre-disease condition.

The present invention further provides in an electronic system and/or in a network, a method for determining whether a subject has a TWIK-8-associated disease or disorder or a pre-disposition to a disease associated with a TWIK-8 wherein the method comprises the steps of determining TWIK-8 sequence information associated with the subject, and based on the TWIK-8 sequence information, determining whether the subject has a TWIK-8-associated disease or disorder or a pre-disposition to a TWIK-8-associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a TWIK-8-associated disease or disorder or a pre-disposition to a TWIK-8 associated disease or disorder associated with TWIK-8, said method comprising the steps of receiving TWIK-8 sequence information from the subject and/or information related thereto, receiving phenotypic information associated with the subject, acquiring information from the network corresponding to TWIK-8 and/or a TWIK-8-associated disease or disorder, and based on one or more of the phenotypic information, the TWIK-8 information (e.g., sequence information and/or information related thereto), and the acquired information, determining whether the subject has a TWIK-8-associated disease or disorder or a pre-disposition to a TWIK-8-associated disease or disorder (e.g., a pain disorder, a cardiovascular disorder, a CNS disorder, or a cellular proliferation, growth, differentiation, or migration disorder). The method may further comprise the step of recommending a particular treatment for the disease, disorder or pre-disease condition.

The present invention also provides a business method for determining whether a subject has a TWIK-8-associated disease or disorder or a pre-disposition to a TWIK-8-associated disease or disorder, said method comprising the steps of receiving information related to TWIK-8 (e.g., sequence information and/or information related thereto), receiving phenotypic information associated with the subject, acquiring information from the network related to TWIK-8 and/or related to a TWIK-8-associated disease or disorder, and based on one or more of the phenotypic information, the TWIK-8 information, and the acquired information, determining whether the subject has a TWIK-8-associated disease or disorder or a pre-disposition to a TWIK-8-associated disease or disorder. The method may further comprise the step of recommending a particular treatment for the disease, disorder or pre-disease condition.

The invention also includes an array comprising a TWIK-8 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be TWIK-8. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a TWIK-8-associated disease or disorder, progression of TWIK-8-associated disease or disorder, and processes, such a cellular transformation associated with the TWIK-8-associated disease or disorder.

The array is also useful for ascertaining the effect of the expression of a gene on the expression of other genes in the same cell or in different cells (e.g., ascertaining the effect of TWIK-8 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including TWIK-8) that could serve as a molecular target for diagnosis or therapeutic intervention.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human TWIK-8 cDNA

In this example, the identification and characterization of the gene encoding human TWIK-8 (clone Fbh12303) is described.

Isolation of the TWIK-8 cDNA

The invention is based, at least in part, on the discovery of a human gene encoding a novel protein, referred to herein as TWIK-8. The entire sequence of the human clone Fbh12303 was determined and found to contain an open reading frame termed human “TWIK-8.”

The nucleotide sequence encoding the human TWIK-8 protein is shown in FIG. 19 and is set forth as SEQ ID NO:28. The protein encoded by this nucleic acid comprises about 419 amino acids and has the amino acid sequence shown in FIG. 19 and set forth as SEQ ID NO:29. The coding region (open reading frame) of SEQ ID NO:28 is set forth as SEQ ID NO:30. Clone Fbh12303, comprising the coding region of human TWIK-8, was deposited with the American Type Culture Collection (ATCC®), 10801 University Boulevard, Manassas, Va. 20110-2209, on ______, and assigned Accession No. ______.

Analysis of the Human TWIK-8 Molecule

The amino acid sequence of human TWIK-8 was analyzed using the program PSORT (http://www.psort.nibb.ac.jp) to predict the localization of the proteins within the cell. This program assesses the presence of different targeting and localization amino acid sequences within the query sequence. The results of the analysis show that human TWIK-8 (SEQ ID NO:29) may be localized to the endoplasmic reticulum or to the mitochondrion.

An analysis of the amino acid sequence of human TWIK-8 using the Signal P program (Henrik, et al. (1997) Protein Engineering 10:1-6), identified the presence of a signal peptide from amino acids 1-46 (FIG. 20).

A search was performed against the Memsat database (FIG. 21) resulting in the identification of six transmembrane domains in the amino acid sequence of the native human TWIK-8 (SEQ ID NO:29) at about residues 32-50, 116-137, 144-165, 195-219, 226-242, and 260-283. This search further identified five transmembrane domains in the amino acid sequence of the predicted mature form of this protein, at about residues 70-91, 98-119, 149-173, 180-196, and 214-237. The results of this search are set forth in FIG. 21.

A search was performed against the HMM database (FIG. 22), resulting in the identification of a “seven-transmembrane receptor domain” from about residues 25-244, and a “cyclic nucleotide-gated channel domain” from about residues 27-204 in the amino acid sequence of human TWIK-8 (SEQ ID NO:29).

A search was also performed against the ProDom database (FIGS. 23A-H), resulting in the identification of “TRAAK potassium channel domains” from about residues 50-104 (score=175), 175-199 (score=115), and 288-382 (score=135); a “potassium channel protein domain” from about residues 99-153 (score=101); a “voltage-gated potassium channel domain” from about residues 102-168 (score=115); an “outward-rectifier TOK1 potassium channel domain” from about residues 215-270 (score=70); and a “potassium channel subunit domain” from about residues 216-287 (score=156) in the amino acid sequence of human TWIK-8 (SEQ ID NO:29).

A BLASTX 2.0 search against the NRP/protot database, using a score of 100, a wordlength of 3, and a Blosum 62 matrix (Altschul et al. (1990) J. Mol. Biol. 215:403), of the translated nucleotide sequence of human TWIK-8 revealed that human TWIK-8 has limited sequence homology to Mus musculus TRAAK K+ channel subunit mRNA (Accession Number AF056492), to Homo sapiens TREK-1 potassium channel (KCNK2) mRNA (Accession Number AF129399), to Mus musculus TREK-1 K+ channel subunit mRNA (Accession Number U73488), and to Homo sapiens two-pore potassium channel TPKC1 mRNA (Accession number AF004711).

Tissue Distribution of TWIK-8 mRNA by PCR and In Situ Hybridization

The tissue distribution of TWIK-8 mRNA is determined by Polymerase Chain Reaction (PCR) on cDNA libraries of normal human tissues using oligonucleotide primers based on the human TWIK-8 sequence.

The tissue distribution of TWIK-8 mRNA is also determined using in situ hybridization. For in situ analysis, various tissues, e.g. tissues obtained from brain, are first frozen on dry ice. Ten-micrometer-thick sections of the tissues are postfixed with 4% formaldehyde in DEPC treated 1×phosphate-buffered saline at room temperature for 10 minutes before being rinsed twice in DEPC 1×phosphate-buffered saline and once in 0.1 M triethanolamine-HCl (pH 8.0). Following incubation in 0.25% acetic anhydride-0.1 M triethanolamine-HCl for 10 minutes, sections are rinsed in DEPC 2×SSC (1×SSC is 0.15M NaCl plus 0.015M sodium citrate). Tissue is then dehydrated through a series of ethanol washes, incubated in 100% chloroform for 5 minutes, and then rinsed in 100% ethanol for 1 minute and 95% ethanol for 1 minute and allowed to air dry.

Hybridizations are performed with ³⁵S-radiolabeled (5×10⁷ cpm/ml) cRNA probes. Probes are incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides are washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides are then rinsed with 2×SSC at room temperature, washed with 2×SSC at 50° C. for 1 hour, washed with 0.2×SSC at 55° C. for 1 hour, and 0.2×SSC at 60° C. for 1 hour. Sections are then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

Tissue Distribution of Human TWIK-8 mRNA Using Tagman™ Analysis

This example describes the tissue distribution of human TWIK-8 mRNA in a variety of cells and tissues, as determined using the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., various human tissue samples, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

Highest expression of TWIK-8 mRNA was detected in cortex, followed by dorsal root ganglia, and hypothalamus. Weak expression was also detected in erythroid tissue, followed by HUVEC, spinal cord, hemangioma, kidney, normal ovary and ovary tumor, megakaryocytes, normal prostate and prostate tumor. Weak expression was also detected in breast tumor although no expression was detected in normal breast tissue.

### Example 2

### Expression of Recombinant TWIK-8 Protein in Bacterial Cells

In this example, TWIK-8 is expressed as a recombinant glutathione-S-transferase (GST) fusion polypeptide in E. coli and the fusion polypeptide is isolated and characterized. Specifically, TWIK-8 is fused to GST and this fusion polypeptide is expressed in E. coli, e.g., strain PEB199. Expression of the GST-TWIK-8 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant TWIK-8 Protein in COS Cells

To express the TWIK-8 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire TWIK-8 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the TWIK-8 DNA sequence is amplified by PCR using two primers. The 5′ primer contains the restriction site of interest followed by approximately twenty nucleotides of the TWIK-8 coding sequence starting from the initiation codon; the 3′ end sequence contains complementary sequences to the other restriction site of interest, a translation stop codon, the HA tag or FLAG tag and the last 20 nucleotides of the TWIK-8 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the TWIK-8 gene is inserted in the correct orientation. The ligation mixture is transformed into E. coli cells (strains HB101, DH5 α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the TWIK-8-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the TWIK-8 polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the TWIK-8 coding sequence is cloned directly into the polylinker of the pCDNA/Amp vector using the appropriate restriction sites. The resulting plasmid is transfected into COS cells in the manner described above, and the expression of the TWIK-8 polypeptide is detected by radiolabeling and immunoprecipitation using a TWIK-8 specific monoclonal antibody.

### V. 47611, A NOVEL HUMAN ION CHANNEL AND USES THEREOF

### Background of the Invention

The ion channel family of proteins is a large family of membrane-bound proteins responsible for a wide range of important transport and signaling functions in cells. The ion channel family includes at least three subfamilies: calcium ion channels (i.e., Ca channels), potassium channels (i e., K channels) and sodium channels (Na channels). Members of the ion channel family are characterized by the presence of six (6) transmembrane helices in which the last two helices flank a loop which determines ion selectivity. In some subfamilies (e.g., Na channels) the domain is repeated four times, whereas in others (e.g., K channels) the protein forms as a tetramer in the membrane.

Calcium channel proteins are involved in the control of neurotransmitter release from neurons (Williams et al. (1992) Science 257:389-395), and play an important role in the regulation of a variety of cellular functions, including membrane excitability, muscle contraction and synaptic transmission (Mori et al. (1991) Nature 350:398-402). The calcium channel proteins are composed of four (4) tightly-coupled subunits (α1, α2, β and γ), the α1 subunit from each creating the pore for the import of extracellular calcium ions. The α1 subunit shares sequence characteristics with all voltage-dependent cation channels, and exploits the same 6-helix bundle structural motif. In both sodium and calcium channels, this motif is repeated 4 times within the sequence to give a 24-helix bundle. There are several tissue-specific pharmacologically and electrophysiologically distinct isoforms of calcium channels, coded for by separate genes in a multi-gene family. In skeletal muscle, each tightly-bound assembly of α, β and γ subunits associates with 4 others to form a pentameric macromolecule (Koch et al. (1990) J. Biol. Chem. 265:17786-17791). Examples of calcium channels include, but are not limited to, the low-voltage-gated channels and the high-voltage-gated channels. Calcium channels are described in, for example, Davila et al. (1999) Ann. N.Y. Acad. Sci. 868:102-17 and McEnery et al. (1998) J. Bioenergetics and Biomembranes 30(4):409-418, the contents of which are incorporated herein by reference.

Sodium channels are transmembrane (TM) voltage-dependent proteins responsible for the depolarizing phase of the action potential in most electrically excitable cells (George et al. (1992) Proc. Natl. Acad Sci. USA 89:4893-4897). They may exist in 3 states (Noda et al. (1984) Nature 312:121-127): the resting state, where the channel is closed; the activated state, where the channel is open; and the inactivated state, where the channel is closed. Several different structurally and functionally distinct isoforms are found in mammals, coded for by a multigene family (Rogart et al. (1989) Proc. Natl. Acad. Sci USA 86:8170-8174), these being responsible for the different types of sodium ion currents found in excitable tissues. The structure of sodium channels is based on 4 internal repeats of a 6-helix bundle (Noda et al. (1986) Nature 320:188-192) (in which 5 of the membrane-spanning segments are hydrophobic and the other is positively charged), forming a 24-helical bundle. The charged segments are believed to be localized within clusters formed by their 5 hydrophobic neighbors. It is postulated that the charged domain may be the voltage sensor region, possibly moving outward on depolarization, causing a conformational change. This model, proposed by (Noda et al., supra), contrasts with that of Sato and Matsumoto (1992) Biochem. Biophys. Res. Commun.. 186:1158-1167), in which the TM segments are juxtaposed octagonally. The basic structural motif (the 6-helix bundle) is also found in potassium and calcium channels.

Potassium channels are the most diverse group of the ion channel family (possibly as a result of gene duplication and alternative splicing of the genes (Perney and Kaczmarek (1991) Curr. Opin. Cell. Biol. 3:663-670 and Luneau et al. (1991) FEBS Lett. 288:163-167). They are important in shaping the action potential, and in neuronal excitability and plasticity (Tempel et al. (1988) Nature 332:837-839). The potassium channel family is composed of several functionally distinct isoforms, which can be broadly separated into 2 groups (Stuehmer et al. (1989) EMBO J. 8:3225-3244). The first is the practically non-inactivating “delayed” group, the second the rapidly inactivating “transient” group. These are all highly similar proteins, with possibly only small amino acid changes causing the diversity of the voltage-dependent gating mechanism, channel conductance and toxin binding properties. Members of the potassium channel family vary in several ways. Some open in response to depolarization of the plasma membrane; others open in response to hyperpolarization or an increase in intracellular calcium concentration; some can be regulated by binding of a transmitter, together with intracellular kinases; and others are regulated by GTP-binding proteins or other second messengers (Schwarz et al. (1988) Nature 331:137-142 (1988). They are also involved in T-cell activation, and may have a role in target cell lysis by cytotoxic T-lymphocytes (Attali et al. (1992) J. Biol. Chem. 267:8650-8657 (1992). Potassium channels are transmembrane (TM) proteins that contain 6 membrane-spanning α-helical segments, 5 of which are hydrophobic, the other being positively charged. The charged segment is believed to be localized within a cluster formed by the hydrophobic helices. As with Na channels, it is postulated that the charged segment may constitute the voltage sensor region, possibly moving outward on depolarization, causing a conformational change. The 6-helix bundle is a common structural motif in sodium channels (in which it is repeated 4 times within the sequence to form a 24-helix bundle), and in calcium channels (where it also forms a 24-helix bundle, which itself is tightly bound to 3 different subunits).

Ion channels play a role in regulating ion transport and signaling in virtually every cell in the human body.

### Summary of the Invention

The present invention is based, at least in part, on the discovery of novel ion channel family members, referred to herein as ion channel 47611, or “IC47611” nucleic acid and protein molecules. The IC47611 molecules of the present invention are useful as targets for developing modulating agents to regulate a variety of cellular processes, including ion transport (e.g., ion conductance); membrane excitability and/or polarization; synaptic transmission; signal transduction; cell activation, proliferation, growth, differentiation and/or migration; and muscle contraction. Accordingly, in one aspect, this invention provides isolated nucleic acid molecules encoding IC47611 proteins or biologically active portions thereof, as well as nucleic acid fragments suitable as primers or hybridization probes for the detection of IC47611-encoding nucleic acids.

In one embodiment, an IC47611 nucleic acid molecule of the invention is at least 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the nucleotide sequence (e.g., to the entire length of the nucleotide sequence) shown in SEQ ID NO:31 or 33 or the nucleotide sequence of the DNA insert of the plasmid deposited with ATCC as Accession Number ______, or a complement thereof.

In a preferred embodiment, the isolated nucleic acid molecule includes the nucleotide sequence shown SEQ ID NO:31 or 33, or a complement thereof. In another embodiment, the nucleic acid molecule includes SEQ ID NO:33 and nucleotides 1-366 of SEQ ID NO:31. In yet another embodiment, the nucleic acid molecule includes SEQ ID NO:33 and nucleotides 1729-4037 of SEQ ID NO:31. In another preferred embodiment, the nucleic acid molecule consists of the nucleotide sequence shown in SEQ ID NO:31 or 33. In another preferred embodiment, the nucleic acid molecule includes a fragment of at least 50 nucleotides (e.g., 50 contiguous nucleotides) of the nucleotide sequence of SEQ ID NO:31 or 33, or a complement thereof.

In another embodiment, an IC47611 nucleic acid molecule includes a nucleotide sequence encoding a protein having an amino acid sequence sufficiently identical to the amino acid sequence of SEQ ID NO:32 or an amino acid sequence encoded by the DNA insert of the plasmid deposited with ATCC as Accession Number ______. In a preferred embodiment, an IC47611 nucleic acid molecule includes a nucleotide sequence encoding a protein having an amino acid sequence at least 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the entire length of the amino acid sequence of SEQ ID NO:32 or the amino acid sequence encoded by the DNA insert of the plasmid deposited with ATCC as Accession Number ______.

In another preferred embodiment, an isolated nucleic acid molecule encodes the amino acid sequence of human IC47611. In yet another preferred embodiment, the nucleic acid molecule includes a nucleotide sequence encoding a protein having the amino acid sequence of SEQ ID NO:32 or the amino acid sequence encoded by the DNA insert of the plasmid deposited with ATCC as Accession Number ______. In yet another preferred embodiment, the nucleic acid molecule is at least 50 nucleotides in length. In a further preferred embodiment, the nucleic acid molecule is at least 50 nucleotides in length and encodes a protein having an IC47611 activity (as described herein).

Another embodiment of the invention features nucleic acid molecules, preferably IC47611 nucleic acid molecules, which specifically detect IC47611 nucleic acid molecules relative to nucleic acid molecules encoding non-IC47611 proteins. For example, in one embodiment, such a nucleic acid molecule is at least 50-100, 100-150, 150-200, 200-216, 216-250, 250-300, 300-340, 340-340, 340-350, 350-400, 400-438, 438-450, 450-500, 500-528, 528-542, 542-550, 550-600, 600-650, 650-700, 700-750, 750-764, 764-800, 800-841, 841-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1950-2000, 2000-2050, 2050-2100, 2100-2150, 2150-2200, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150 -3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000 or more nucleotides in length and hybridizes under stringent conditions to a nucleic acid molecule comprising the nucleotide sequence shown in SEQ ID NO:31, the nucleotide sequence of the DNA insert of the plasmid deposited with ATCC as Accession Number ______, or a complement thereof.

In preferred embodiments, the nucleic acid molecules are at least 15 (e.g., 15 contiguous) nucleotides in length and hybridize under stringent conditions to SEQ ID NO:31.

In other preferred embodiments, the nucleic acid molecule encodes a naturally occurring allelic variant of a polypeptide comprising the amino acid sequence of SEQ ID NO:32 or an amino acid sequence encoded by the DNA insert of the plasmid deposited with ATCC as Accession Number ______, wherein the nucleic acid molecule hybridizes to a nucleic acid molecule comprising SEQ ID NO:31 or 33 under stringent conditions.

Another embodiment of the invention provides an isolated nucleic acid molecule which is antisense to an IC47611 nucleic acid molecule, e.g., the coding strand of an IC47611 nucleic acid molecule.

Another aspect of the invention provides a vector comprising an IC47611 nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably an IC47611 protein, by culturing in a suitable medium, a host cell, e.g., a mammalian host cell such as a non-human mammalian cell, of the invention containing a recombinant expression vector, such that the protein is produced.

Another aspect of this invention features isolated or recombinant IC47611 proteins and polypeptides. In one embodiment, an isolated IC47611 protein has one or more of the following domains: a transmembrane domain, an inward-rectifier potassium channel (IRK) domain, and an inward-rectifier potassium channel (IRK)-related domain. In a preferred embodiment, an IC47611 protein includes at least one or more of the following domains: a transmembrane domain, an inward-rectifier potassium channel (IRK) domain, and an inward-rectifier potassium channel (IRK)-related domain, and has an amino acid sequence at least about 50%, 55%, 60%, 65%, 67%, 68%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the amino acid sequence of SEQ ID NO:32, or the amino acid sequence encoded by the DNA insert of the plasmid deposited with ATCC as Accession Number ______. In another preferred embodiment, an IC47611 protein includes at least one transmembrane domain and has an IC47611 activity (as described herein).

In yet another preferred embodiment, an IC47611 protein includes one or more of the following domains: a transmembrane domain, an inward-rectifier potassium channel (IRK) domain, and an inward-rectifier potassium channel (IRK)-related domain, and is encoded by a nucleic acid molecule having a nucleotide sequence which hybridizes under stringent hybridization conditions to a nucleic acid molecule comprising the nucleotide sequence of SEQ ID NO:31 or 33.

In another embodiment, the invention features fragments of the protein having the amino acid sequence of SEQ ID NO:32, wherein the fragment comprises at least 15 amino acids (e.g., contiguous amino acids) of the amino acid sequence of SEQ ID NO:32, or an amino acid sequence encoded by the DNA insert of the plasmid deposited with the ATCC as Accession Number ______. In another embodiment, an IC47611 protein has the amino acid sequence of SEQ ID NO:32.

In another embodiment, the invention features an IC47611 protein which is encoded by a nucleic acid molecule consisting of a nucleotide sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to a nucleotide sequence of SEQ ID NO:31 or 33, or a complement thereof. This invention further features an IC47611 protein, which is encoded by a nucleic acid molecule consisting of a nucleotide sequence which hybridizes under stringent hybridization conditions to a nucleic acid molecule comprising the nucleotide sequence of SEQ ID NO:31 or 33, or a complement thereof.

The proteins of the present invention or portions thereof, e.g., biologically active portions thereof, can be operatively linked to a non-IC47611 polypeptide (e.g., heterologous amino acid sequences) to form fusion proteins. The invention further features antibodies, such as monoclonal or polyclonal antibodies, that specifically bind proteins of the invention, preferably IC47611 proteins. In addition, the IC47611 proteins or biologically active portions thereof can be incorporated into pharmaceutical compositions, which optionally include pharmaceutically acceptable carriers.

In another aspect, the present invention provides a method for detecting the presence of an IC47611 nucleic acid molecule, protein, or polypeptide in a biological sample by contacting the biological sample with an agent capable of detecting an IC47611 nucleic acid molecule, protein, or polypeptide such that the presence of an IC47611 nucleic acid molecule, protein or polypeptide is detected in the biological sample.

In another aspect, the present invention provides a method for detecting the presence of IC47611 activity in a biological sample by contacting the biological sample with an agent capable of detecting an indicator of IC47611 activity such that the presence of IC47611 activity is detected in the biological sample.

In another aspect, the invention provides a method for modulating IC47611 activity comprising contacting a cell capable of expressing IC47611 with an agent that modulates IC47611 activity such that IC47611 activity in the cell is modulated. In one embodiment, the agent inhibits IC47611 activity. In another embodiment, the agent stimulates IC47611 activity. In one embodiment, the agent is an antibody that specifically binds to an IC47611 protein. In another embodiment, the agent modulates expression of IC47611 by modulating transcription of an IC47611 gene or translation of an IC47611 mRNA. In yet another embodiment, the agent is a nucleic acid molecule having a nucleotide sequence that is antisense to the coding strand of an IC47611 mRNA or an IC47611 gene.

In one embodiment, the methods of the present invention are used to treat a subject having a disorder characterized by aberrant or unwanted IC47611 protein or nucleic acid expression or activity by administering an agent which is an IC47611 modulator to the subject. In one embodiment, the IC47611 modulator is an IC47611 protein. In another embodiment the IC47611 modulator is an IC47611 nucleic acid molecule. In yet another embodiment, the IC47611 modulator is a peptide, peptidomimetic, or other small molecule. In a preferred embodiment, the disorder characterized by aberrant or unwanted IC47611 protein or nucleic acid expression is a CNS disorder, such as a cognitive or neurodegenerative disorder. In another preferred embodiment, the disorder characterized by aberrant or unwanted IC47611 protein or nucleic acid expression is a cardiovascular disorder. In another preferred embodiment, the disorder characterized by aberrant or unwanted IC47611 protein or nucleic acid expression is a muscular disorder. In another embodiment, the disorder characterized by aberrant or unwanted IC47611 activity is a pain disorder. In another embodiment, the disorder characterized by aberrant or unwanted IC47611 activity is a cell proliferation, growth, differentiation, or migration disorder.

The present invention also provides diagnostic assays for identifying the presence or absence of a genetic alteration characterized by at least one of (i) aberrant modification or mutation of a gene encoding an IC47611 protein; (ii) mis-regulation of the gene; and (iii) aberrant post-translational modification of an IC47611 protein, wherein a wild-type form of the gene encodes a protein with an IC47611 activity.

In another aspect the invention provides methods for identifying a compound that binds to or modulates the activity of an IC47611 protein, by providing an indicator composition comprising an IC47611 protein having IC47611 activity, contacting the indicator composition with a test compound, and determining the effect of the test compound on IC47611 activity in the indicator composition to identify a compound that modulates the activity of an IC47611 protein.

Other features and advantages of the invention will be apparent from the following detailed description and claims.

### Detailed Description of the Invention

The present invention is based, at least in part, on the discovery of novel molecules, referred to herein as IC47611 (for ion channel 47611) nucleic acid and protein molecules, which are novel members of the ion channel family. These novel molecules are capable of, for example, modulating ion transport in an electrically excitable cell (e.g., a neuronal or muscle (e.g., cardiac muscle) cell), or in a non-electrically excitable cell, e.g., a spleen cell.

As used herein, the term “ion channel” includes a protein or polypeptide which is involved in receiving, conducting, and transmitting signals in an cell (e.g., an electrically excitable cell, for example, a neuronal or muscle cell). Ion channels can determine membrane excitability (the ability of, for example, a cell to respond to a stimulus and to convert it into a sensory impulse). Ion channels can also influence the resting potential of membranes, wave forms and frequencies of action potentials, and thresholds of excitation. Ion channels are typically expressed in electrically excitable cells, e.g., neuronal cells, and may form heteromultimeric structures (e.g., composed of more than one type of subunit). Ion channels may also be found in non-excitable cells (e.g., endothelial cells or spleen cells), where they may play a role in, for example, signal transduction. As the IC47611 molecules of the present invention may modulate ion channel mediated activities, they may be useful for developing novel diagnostic and therapeutic agents for ion channel associated disorders.

As used herein, an “ion channel associated disorder” includes a disorder, disease or condition which is characterized by a misregulation of an ion channel mediated activity. Ion channel associated disorders can detrimentally affect conveyance of sensory impulses from the periphery to the brain and/or conductance of motor impulses from the brain to the periphery; integration of reflexes; interpretation of sensory impulses; cellular proliferation, growth, differentiation, or migration, and emotional, intellectual (e.g., learning and memory), or motor processes. Examples of ion channel associated disorders include CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Ion channel disorders also include pain disorders. The IC47611 molecules of the present invention may be present on sensory neurons and, thus, may be involved in detecting, for example, noxious chemical, mechanical, or thermal stimuli and transducing this information into membrane depolarization events. Thus, the IC47611 molecules by participating in pain signaling mechanisms, may modulate pain elicitation and act as targets for developing novel diagnostic targets and therapeutic agents to control pain.

Further examples of ion channel associated disorders include cardiac-related disorders. Cardiovascular system disorders in which the IC47611 molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia. IC47611-mediated or related disorders also include disorders of the musculoskeletal system such as paralysis and muscle weakness, e.g., ataxia, myotonia, and myokymia.

Ion channel disorders also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The IC47611 molecules of the present invention are involved in signal transduction mechanisms, which are known to be involved in cellular growth, differentiation, and migration processes. Thus, the IC47611 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementia; hepatic disorders; cardiovascular disorders; and hematopoietic and/or myeloproliferative disorders.

IC47611-associated or related disorders also include disorders of tissues in which IC47611 protein is expressed.

As used herein, an “ion channel mediated activity” includes an activity which involves an ion channel, e.g., an ion channel associated with receiving, conducting, and transmitting signals, in electrically excitable or non-electrically excitable cells. Ion channel mediated activities include release of neurotransmitters or second messenger molecules, e.g., dopamine or norepinephrine, from cells, e.g., neuronal cells; modulation of resting potential of membranes, wave forms and frequencies of action potentials, and thresholds of excitation; participation in signal transduction pathways; and modulation of processes such as integration of sub-threshold synaptic responses and the conductance of back-propagating action potentials in, for example, neuronal cells (e.g., changes in those action potentials resulting in a morphological or differentiative response in the cell).

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., monkey proteins. Members of a family may also have common functional characteristics.

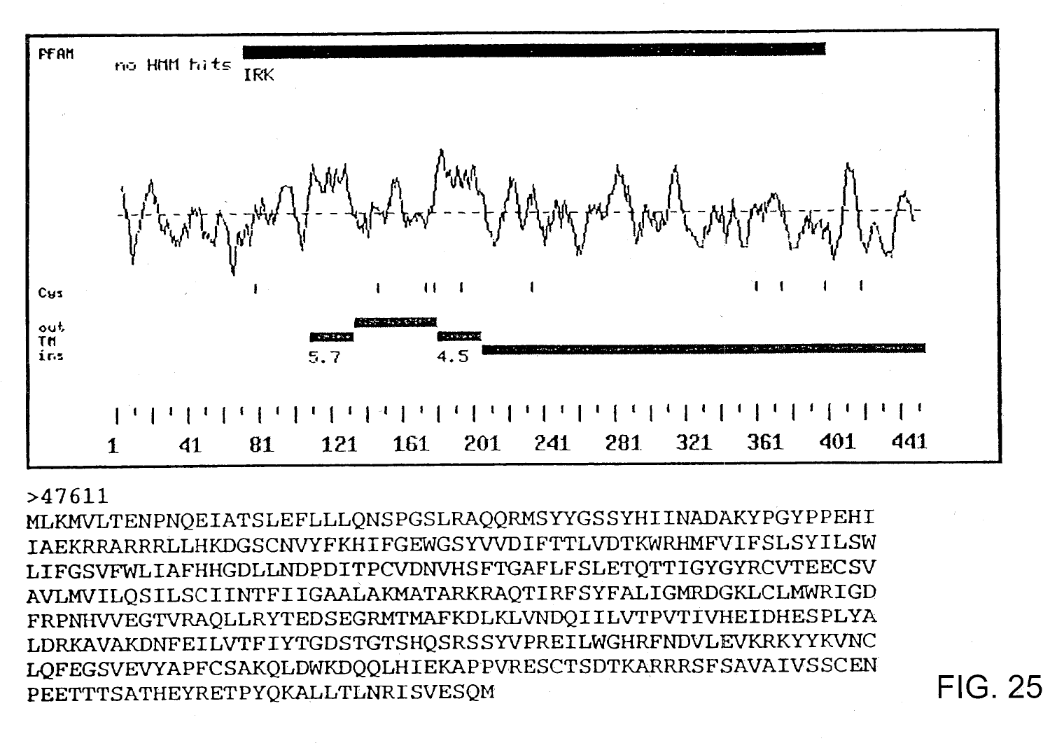

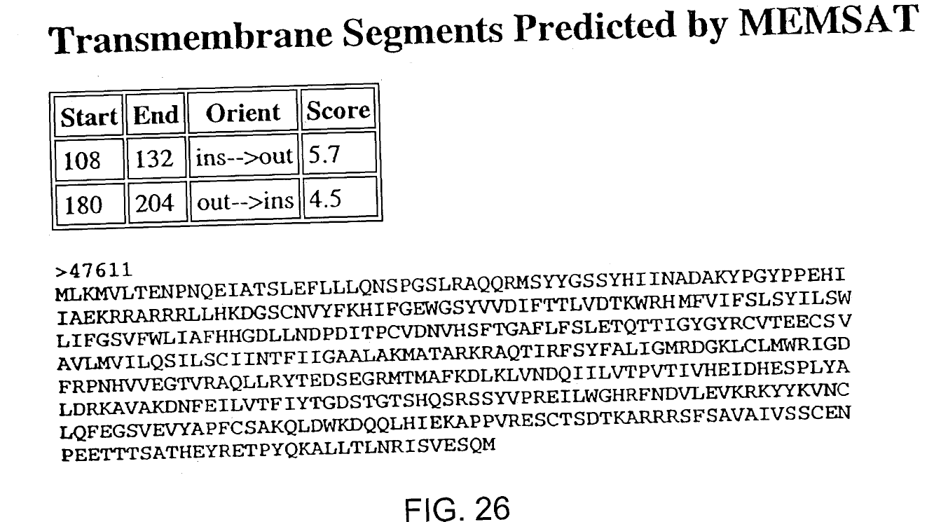

For example, the family of IC47611 proteins comprises at least one “transmembrane domain” and preferably two transmembrane domains. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al., (1996) Annu. Rev. Neurosci. 19: 235-263, the contents of which are incorporated herein by reference. Amino acid residues 108-132 and 180-204 of the native IC47611 protein are predicted to comprise transmembrane domains (see FIGS. 25 and 26). Accordingly, IC47611 proteins having at least 50-60% homology, preferably about 60-70%, more preferably about 70-80%, or about 80-90% homology with a transmembrane domain of human IC47611 are within the scope of the invention.

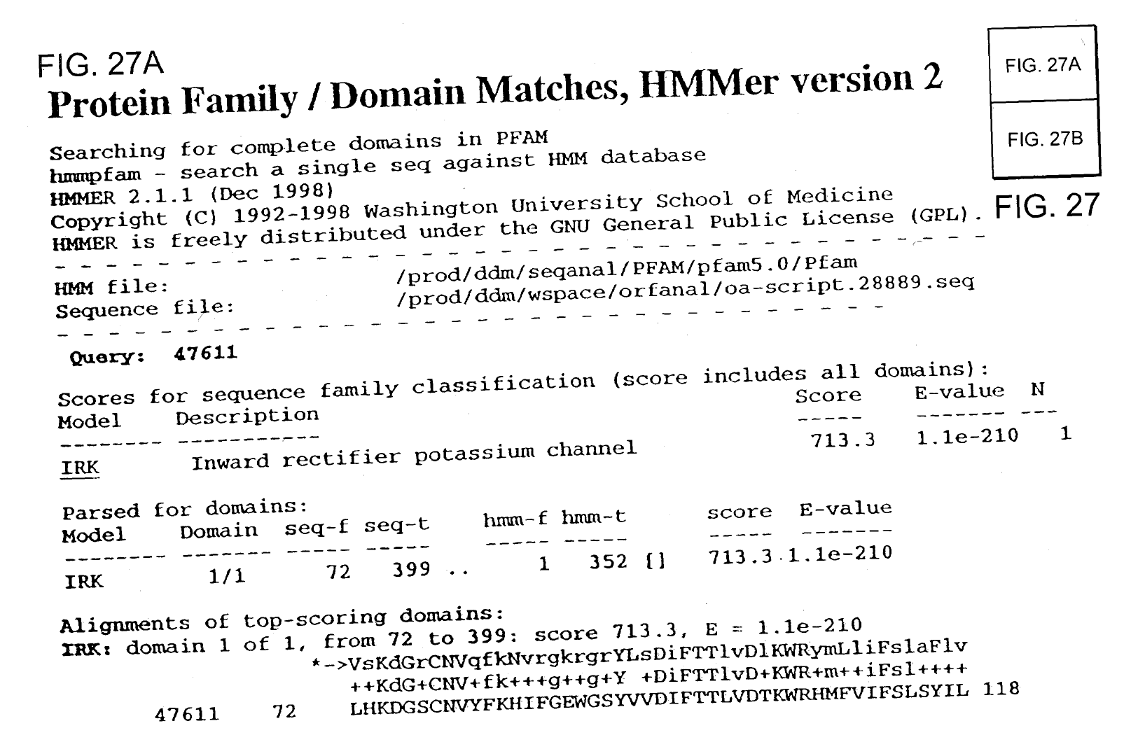

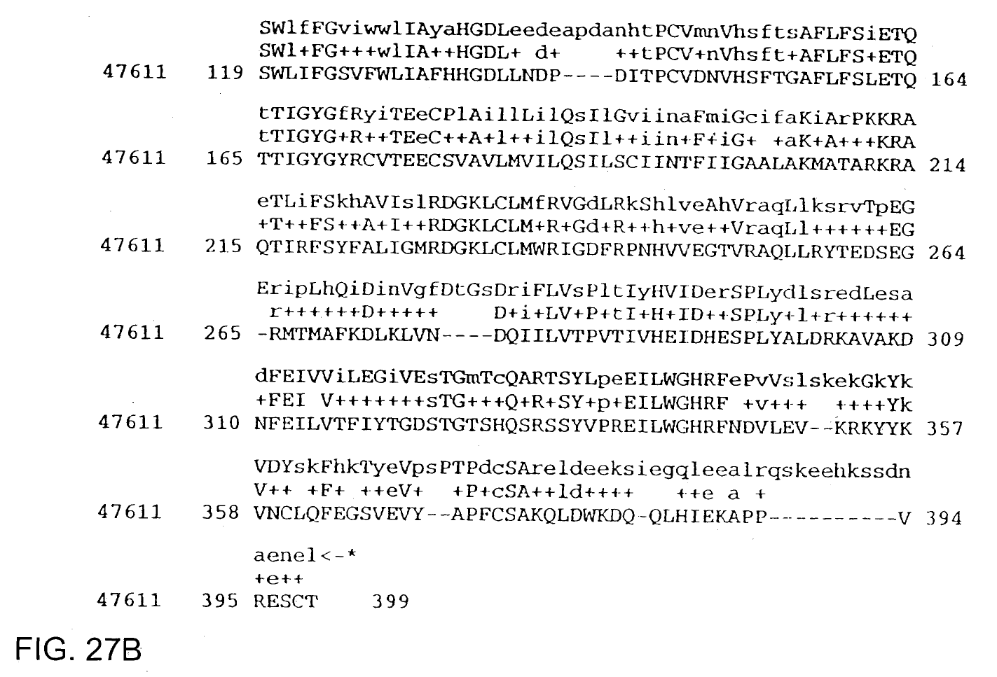

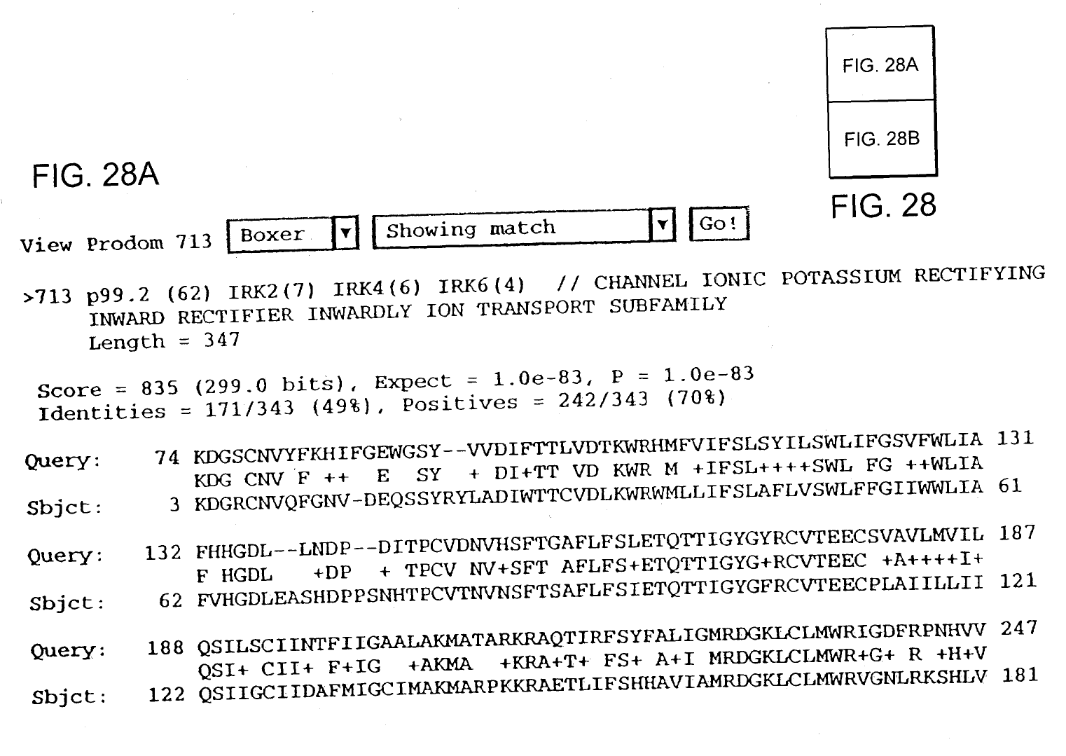

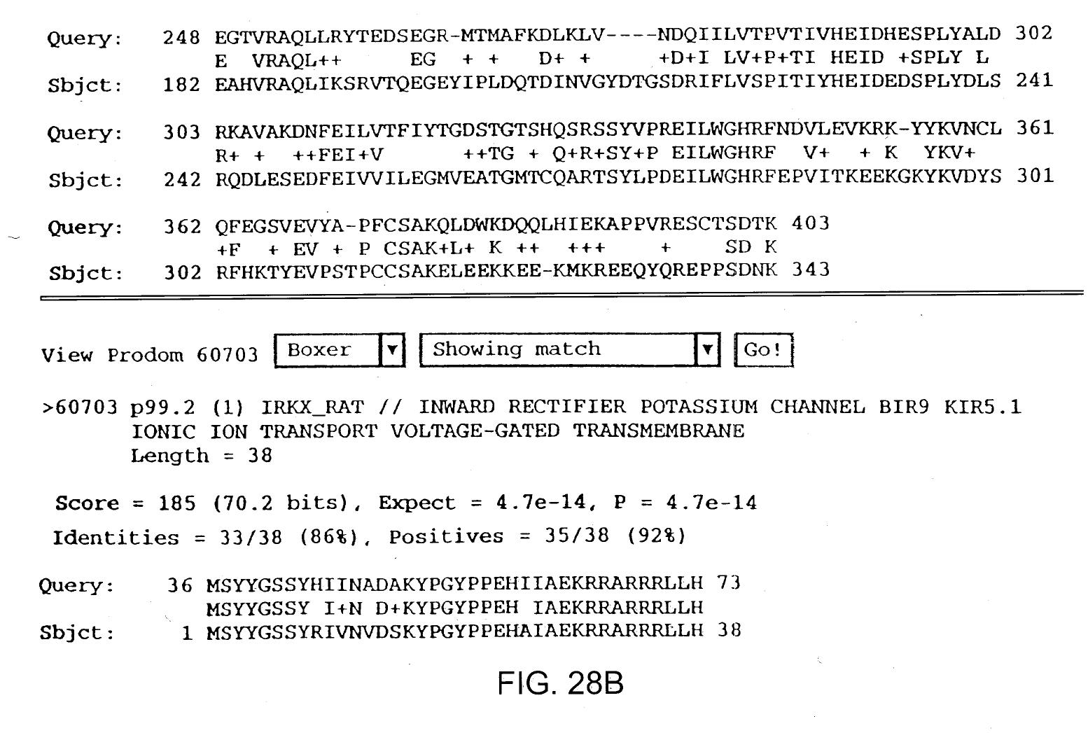

In another embodiment, an IC47611 molecule of the present invention is identified based on the presence of an “an inward-rectifier potassium channel (IRK) domain” in the protein or corresponding nucleic acid molecule. Inward-rectifier potassium channel (IRK) domains are described in Doupnik et al. (1995) Curr. Opin. Neurobiol. 5: 268-277. As used herein, the term “inward-rectifier potassium channel (IRK) domain” includes a protein domain having an amino acid sequence of about 250-400 amino acid residues and having a bit score for the alignment of the sequence to the inward-rectifier potassium channel (IRK) domain of at least 713-835. Preferably, an inward-rectifier potassium channel (IRK) domain includes at least about 300-350, or more preferably about 328-330 amino acid residues, and has a bit score for the alignment of the sequence to the inward-rectifier potassium channel (IRK) domain of at least 50, 100, 150, 200, 250, 300, 350, 400, 450, 500, 550, 600, 650, 700 or higher. The inward-rectifier potassium channel (IRK) domain has been assigned Pfam accession number PF01007 (http:/Hgenome.wustl.edu/Pfam/html). To identify the presence of an inward-rectifier potassium channel (IRK) domain in an IC47611 protein, and make the determination that a protein of interest has a particular profile, the amino acid sequence of the protein is searched against a database of known protein domains (e.g., the HMM database) using the default parameters. A search was performed against the HMM database resulting in the identification of an inward-rectifier potassium channel (IRK) domain in the amino acid sequence of human IC47611 (SEQ ID NO:32) at about residues 72-399 of SEQ ID NO:32. The results of the search are set forth in FIG. 27. A search was also performed against the ProDom database, resulting in the identification of an inward-rectifier potassium channel (IRK) domain in the amino acid sequence of human IC47611 (SEQ ID NO:32) at about residues 74-403 of SEQ ID NO:32. The results of the search are set forth in FIG. 28.

In another embodiment, an IC47611 molecule of the present invention is identified based on the presence of an “inward-rectifier potassium channel (IRK)-related domain” in the protein or corresponding nucleic acid molecule. As used herein, the term “inward-rectifier potassium channel (IRK)-related domain” includes a protein domain having an amino acid sequence of about 20-70 amino acid residues and having a bit score for the alignment of the sequence to the inward-rectifier potassium channel (IRK)-related domain of at least 185. Preferably, an inward-rectifier potassium channel (IRK)-related domain includes at least about 30-50, or more preferably about 38 amino acid residues, and has a bit score for the alignment of the sequence to the IC47611 amino acid sequence of at least 20, 25, 30, 35, 40, 45, 50, 55, 60, 65, 70, 80, 90, 100, 110, 120, 130, 140, 150, 160, 170, 180 or higher. The inward-rectifier potassium channel (IRK)-related domain has been assigned ProDom entry 60703. To identify the presence of an inward-rectifier potassium channel (IRK)-related domain in an IC47611 protein, and make the determination that a protein of interest has a particular profile, the amino acid sequence of the protein is searched against a database of known protein domains (e.g., the ProDom database) using the default parameters (available at http://www.toulouse.inra.fr/prodom.html). A search was performed against the ProDom database resulting in the identification of an inward-rectifier potassium channel (IRK)-related domain in the amino acid sequence of human IC47611 (SEQ ID NO:32) at about residues 36-73 of SEQ ID NO:32. The results of the search are set forth in FIG. 28.

In a preferred embodiment, the IC47611 molecules of the invention include at least one transmembrane domain, an inward-rectifier potassium channel (IRK) domain, and an inward-rectifier potassium channel (IRK)-related domain.

Isolated proteins of the present invention, preferably IC47611 proteins, have an amino acid sequence sufficiently identical to the amino acid sequence of SEQ ID NO:32 or are encoded by a nucleotide sequence sufficiently identical to SEQ ID NO:31 or 33. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, an “IC47611 activity”, “biological activity of IC47611” or “functional activity of IC47611”, refers to an activity exerted by an IC47611 protein, polypeptide or nucleic acid molecule on an IC47611 responsive cell or tissue, or on an IC47611 protein substrate, as determined in vivo, or in vitro, according to standard techniques. In one embodiment, an IC47611 activity is a direct activity, such as an association with an IC47611-target molecule. As used herein, a “target molecule” or “binding partner” is a molecule with which an IC47611 protein binds or interacts in nature, such that IC47611-mediated function is achieved. An IC47611 target molecule can be a non-IC47611 molecule or an IC47611 protein or polypeptide of the present invention. In an exemplary embodiment, an IC47611 target molecule is an IC47611 ligand, e.g., an ion channel pore-forming subunit or an ion channel ligand. Alternatively, an IC47611 activity is an indirect activity, such as a cellular signaling activity mediated by interaction of the IC47611 protein with an IC47611 ligand. The biological activities of IC47611 are described herein. For example, the IC47611 proteins of the present invention can have one or more of the following activities: (1) modulation of membrane excitability, (2) modulation of intracellular ion concentration, (3) modulation of membrane polarization (e.g., membrane polarization and/or depolarization), (4) modulation of action potential, (5) modulation of cellular signal transduction, (6) modulation of neurotransmitter release (e.g., from neuronal cells), (7) modulation of synaptic transmission, (8) modulation of neuronal excitability and/or plasticity, (9) modulation of muscle contraction, (10) modulation of cell activation (e.g., T cell activation), and/or (11) modulation of cellular proliferation, growth, migration and/or differentiation.

Accordingly, another embodiment of the invention features isolated IC47611 proteins and polypeptides having an IC47611 activity. Preferred proteins are IC47611 proteins having one or more of the following domains: a transmembrane domain, an inward-rectifier potassium channel (IRK) domain, and an inward-rectifier potassium channel (IRK)-related domain, and, preferably, an IC47611 activity. Yet other preferred proteins are IC47611 proteins having at least one transmembrane domain, an inward-rectifier potassium channel (IRK) domain, and an inward-rectifier potassium channel (IRK)-related domain, and, preferably, an IC47611 activity.

Additional preferred proteins have one or more of the following domains: a transmembrane domain, an inward-rectifier potassium channel (IRK) domain, and an inward-rectifier potassium channel (IRK)-related domain, and are, preferably, encoded by a nucleic acid molecule having a nucleotide sequence which hybridizes under stringent hybridization conditions to a nucleic acid molecule comprising the nucleotide sequence of SEQ ID NO:31 or 33.

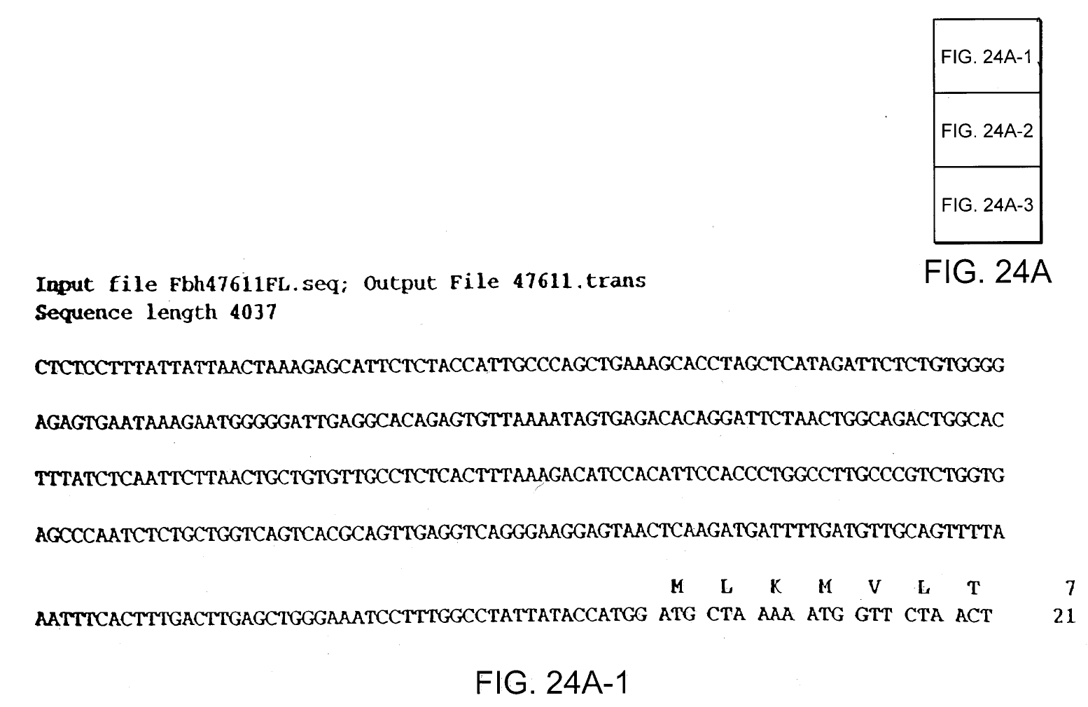

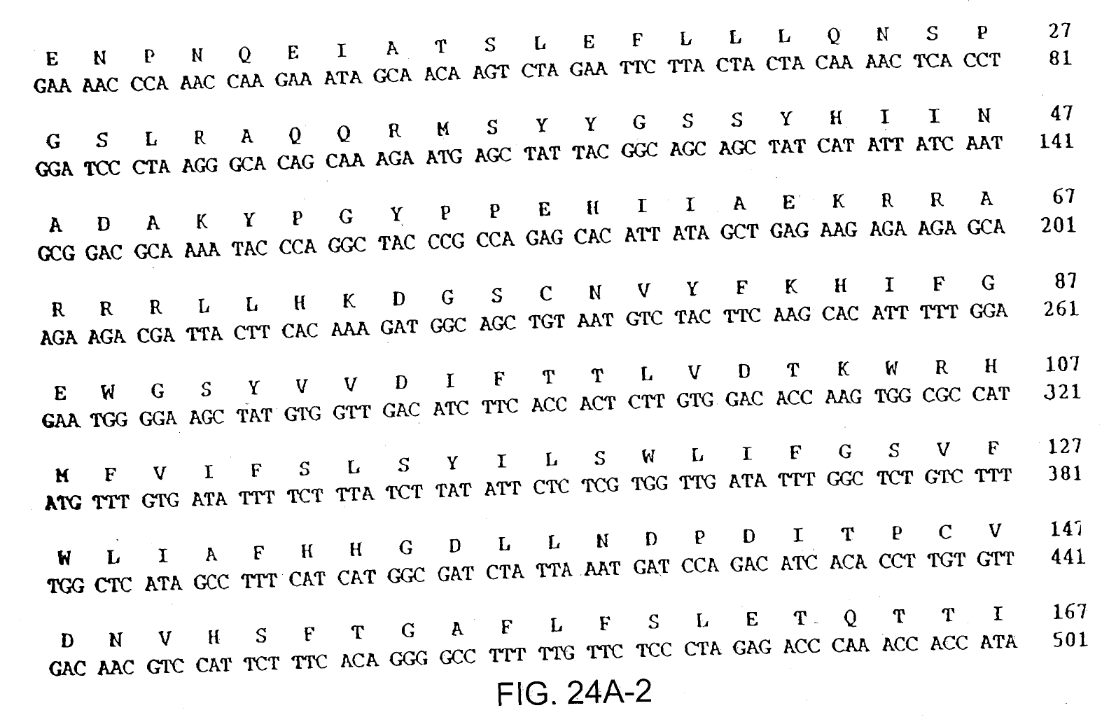

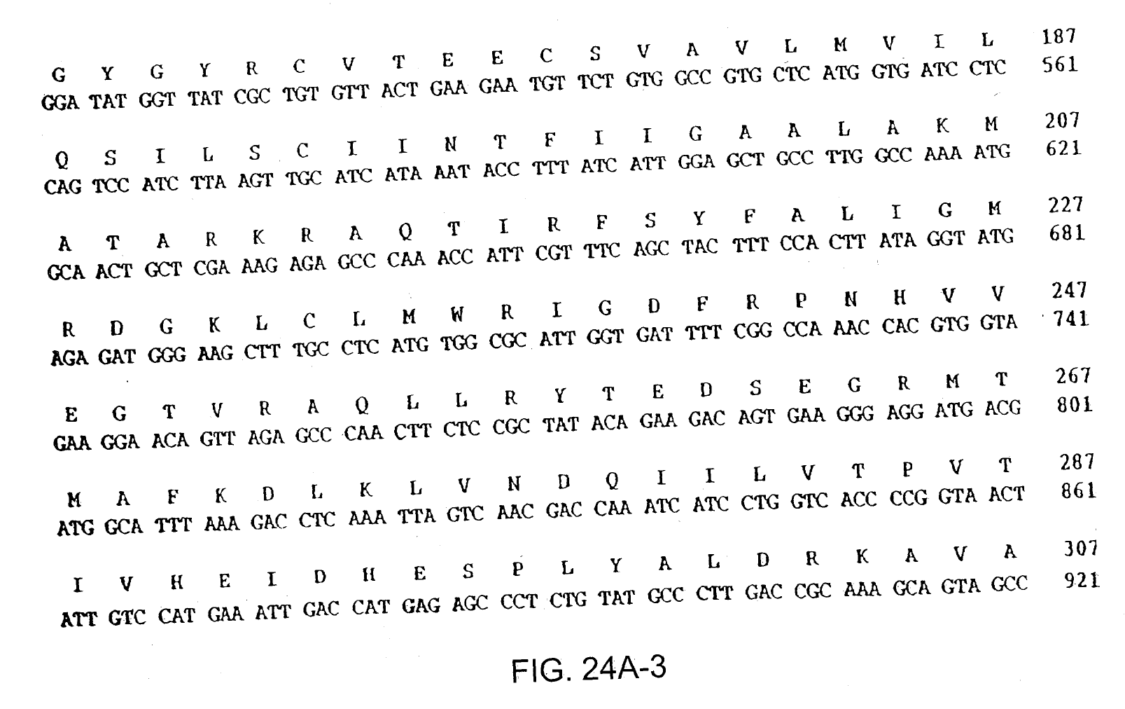

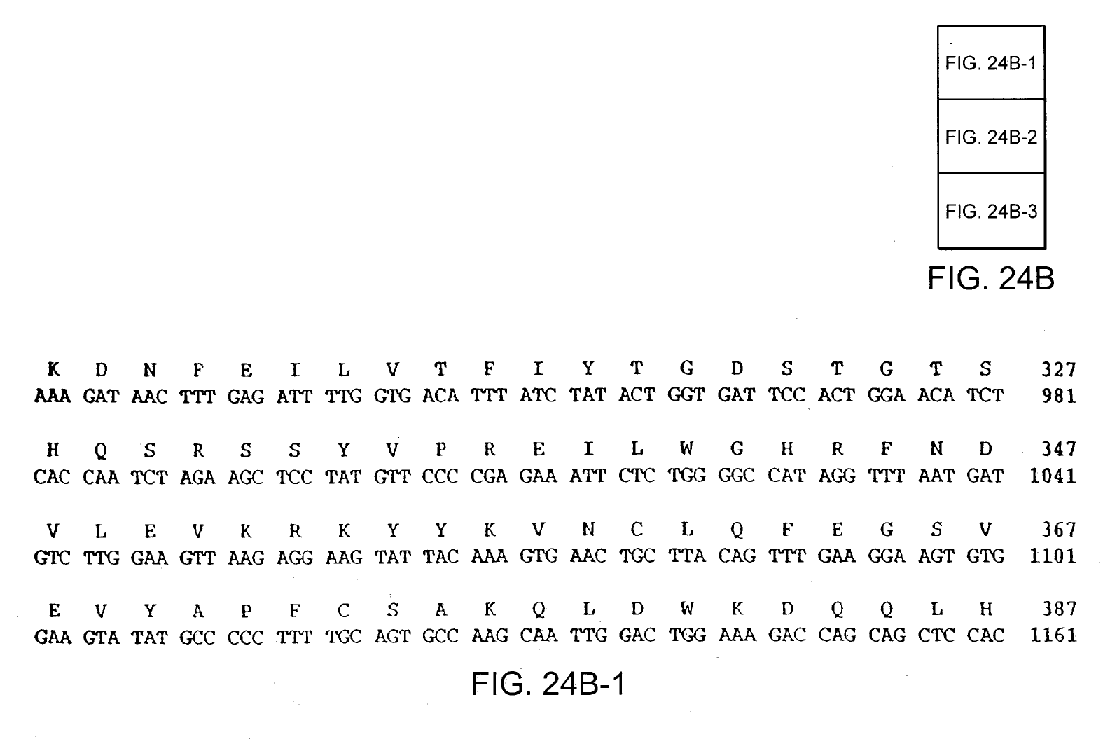

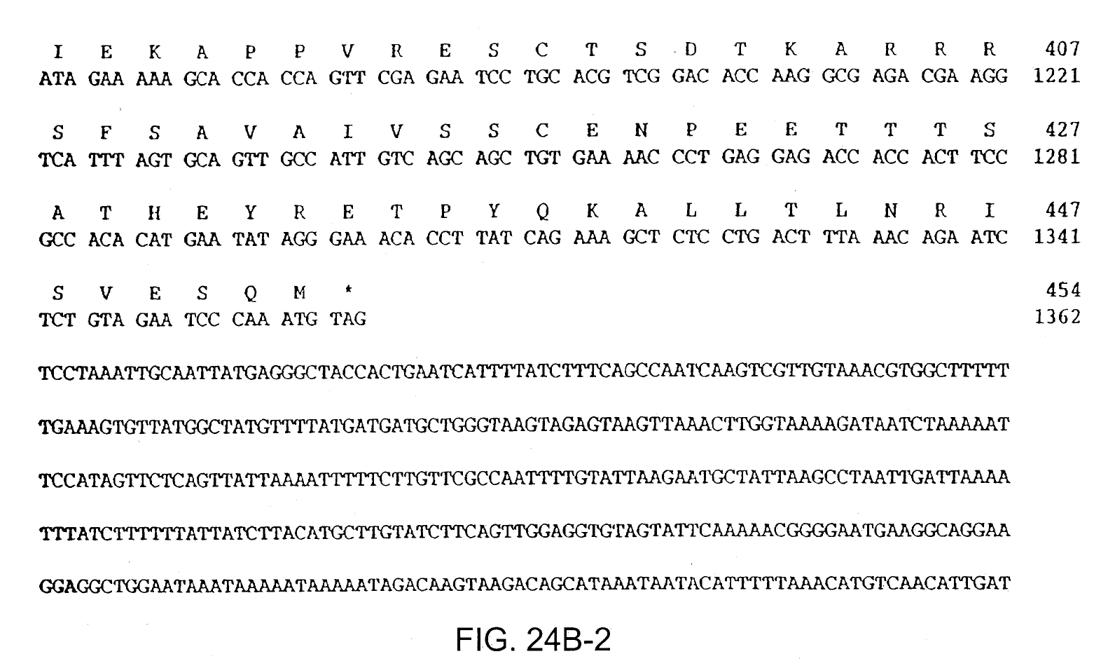

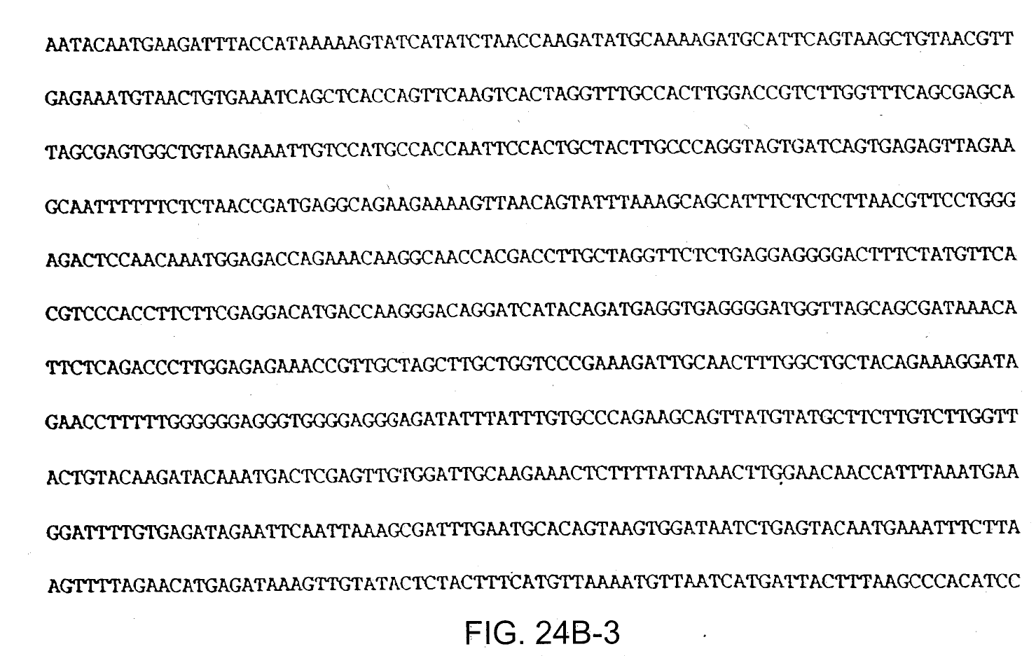

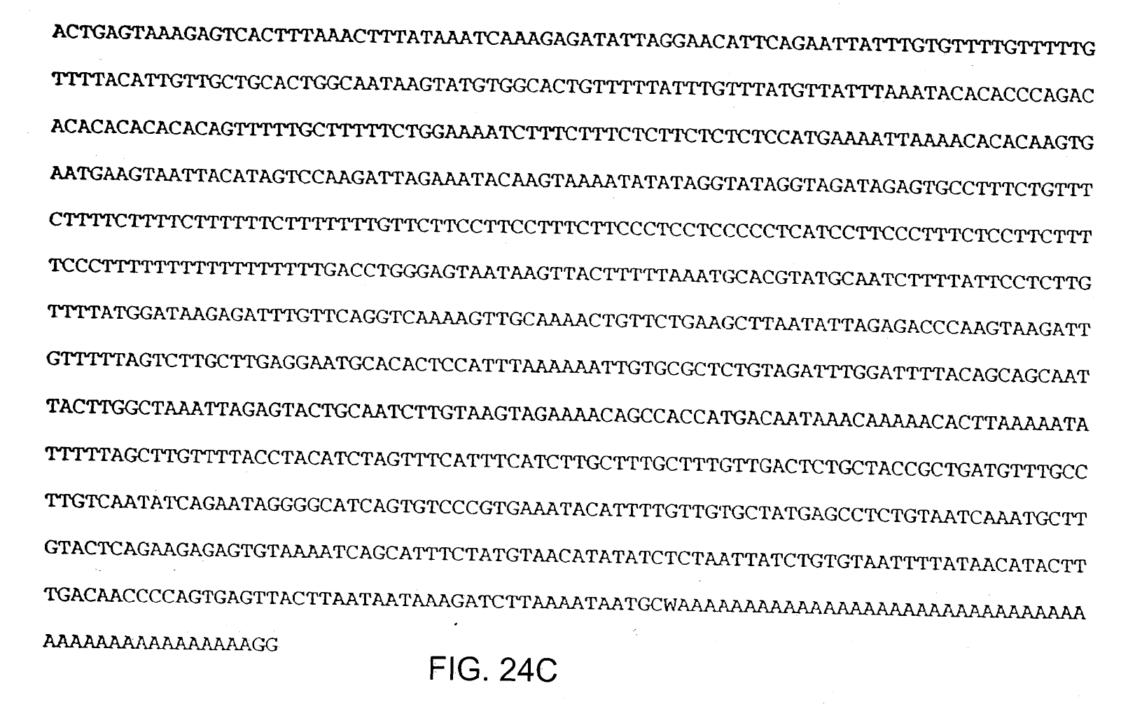

The nucleotide sequence of the isolated human IC47611 cDNA and the predicted amino acid sequence of the human IC47611 polypeptide are shown in FIGS. 24A-C and in SEQ ID NOs:31 and 33, respectively. A plasmid containing the nucleotide sequence encoding human IC47611 was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The human IC47611 gene, which is approximately 4037 nucleotides in length, encodes a protein having a molecular weight of approximately 51.3 kD and which is approximately 453 amino acid residues in length.

Various aspects of the invention are described in further detail in the following subsections:

I. Isolated Nucleic Acid Molecules

One aspect of the invention pertains to isolated nucleic acid molecules that encode IC47611 proteins or biologically active portions thereof, as well as nucleic acid fragments sufficient for use as hybridization probes to identify IC47611-encoding nucleic acid molecules (e.g., IC47611 mRNA) and fragments for use as PCR primers for the amplification or mutation of IC47611 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated IC47611 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:31 or 33, or the nucleotide sequence of the DNA insert of the plasmid deposited with ATCC as Accession Number ______, or a portion thereof, can be isolated using standard molecular biology techniques and the sequence information provided herein. Using all or portion of the nucleic acid sequence of SEQ ID NO:31 or 33, or the nucleotide sequence of the DNA insert of the plasmid deposited with ATCC as Accession Number ______, as a hybridization probe, IC47611 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:31 or 33, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:31 or 33, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to IC47611 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:31. This cDNA comprises sequences encoding the human IC47611 protein (i.e., “the coding region”, from nucleotides 367-1728), as well as 5′ untranslated sequences (nucleotides 1-366) and 3′ untranslated sequences (nucleotides 1729-4037). Alternatively, the nucleic acid molecule can comprise only the coding region of SEQ ID NO:31 (e.g, nucleotides 367-1728, corresponding to SEQ ID NO:33).

In another preferred embodiment, an isolated nucleic acid molecule of the invention comprises a nucleic acid molecule which is a complement of the nucleotide sequence shown in SEQ ID NO:31 or 33, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:31 or 33, or the nucleotide sequence of the DNA insert of the plasmid deposited with ATCC as Accession Number ______, is one which is sufficiently complementary to the nucleotide sequence shown in SEQ ID NO:31 or 33, or the nucleotide sequence of the DNA insert of the plasmid deposited with ATCC as Accession Number ______, such that it can hybridize to the nucleotide sequence shown in SEQ ID NO:31 or 33, or the nucleotide sequence of the DNA insert of the plasmid deposited with ATCC as Accession Number ______, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the entire length of the nucleotide sequence shown in SEQ ID NO:31 or 33, or the entire length of the nucleotide sequence of the DNA insert of the plasmid deposited with ATCC as Accession Number ______, or a portion of any of these nucleotide sequences.

Moreover, the nucleic acid molecule of the invention can comprise only a portion of the nucleic acid sequence of SEQ ID NO:31 or 33, or the nucleotide sequence of the DNA insert of the plasmid deposited with ATCC as Accession Number ______, for example, a fragment which can be used as a probe or primer or a fragment encoding a portion of an IC47611 protein, e.g., a biologically active portion of an IC47611 protein. The nucleotide sequence determined from the cloning of the IC47611 gene allows for the generation of probes and primers designed for use in identifying and/or cloning other IC47611 family members, as well as IC47611 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:31 or 33, or the nucleotide sequence of the DNA insert of the plasmid deposited with ATCC as Accession Number ______, of an anti-sense sequence of SEQ ID NO:31 or 33, or the nucleotide sequence of the DNA insert of the plasmid deposited with ATCC as Accession Number ______, or of a naturally occurring allelic variant or mutant of SEQ ID NO:31 or 33, or the nucleotide sequence of the DNA insert of the plasmid deposited with ATCC as Accession Number ______. In one embodiment, a nucleic acid molecule of the present invention comprises a nucleotide sequence which is greater than 50-100, 100-150, 150-200, 200-216, 216-250, 250-300, 300-340, 340-350, 350-400, 400-438, 438-450, 450-500, 500-528, 528-542, 542-550, 550-600, 600-650, 650-700, 700-750, 750-764, 764-800, 800-841, 841-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1950-2000, 2000-2050, 2050-2100, 2100-2150, 2150-2200, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000 or more nucleotides in length and hybridizes under stringent hybridization conditions to a nucleic acid molecule of SEQ ID NO:31 or 33, or the nucleotide sequence of the DNA insert of the plasmid deposited with ATCC as Accession Number ______.

Probes based on the IC47611 nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an IC47611 protein, such as by measuring a level of an IC47611-encoding nucleic acid in a sample of cells from a subject e.g., detecting IC47611 mRNA levels or determining whether a genomic IC47611 gene has been mutated or deleted.

A nucleic acid fragment encoding a “biologically active portion of an IC47611 protein” can be prepared by isolating a portion of the nucleotide sequence of SEQ ID NO:31 or 33, or the nucleotide sequence of the DNA insert of the plasmid deposited with ATCC as Accession Number ______, which encodes a polypeptide having an IC47611 biological activity (the biological activities of the IC47611 proteins are described herein), expressing the encoded portion of the IC47611 protein (e.g., by recombinant expression in vitro) and assessing the activity of the encoded portion of the IC47611 protein.

The invention further encompasses nucleic acid molecules that differ from the nucleotide sequence shown in SEQ ID NO:31 or 33, or the nucleotide sequence of the DNA insert of the plasmid deposited with ATCC as Accession Number ______, due to degeneracy of the genetic code and thus encode the same IC47611 proteins as those encoded by the nucleotide sequence shown in SEQ ID NO:31 or 33, or the nucleotide sequence of the DNA insert of the plasmid deposited with ATCC as Accession Number ______. In another embodiment, an isolated nucleic acid molecule of the invention has a nucleotide sequence encoding a protein having an amino acid sequence shown in SEQ ID NO:32.

In addition to the IC47611 nucleotide sequences shown in SEQ ID NO:31 or 33, or the nucleotide sequence of the DNA insert of the plasmid deposited with ATCC as Accession Number ______, it will be appreciated by those skilled in the art that DNA sequence polymorphisms that lead to changes in the amino acid sequences of the IC47611 proteins may exist within a population (e.g., the human population). Such genetic polymorphism in the IC47611 genes may exist among individuals within a population due to natural allelic variation. As used herein, the terms “gene” and “recombinant gene” refer to nucleic acid molecules which include an open reading frame encoding an IC47611 protein, preferably a mammalian IC47611 protein, and can further include non-coding regulatory sequences, and introns.

Allelic variants of human IC47611 include both functional and non-functional IC47611 proteins. Functional allelic variants are naturally occurring amino acid sequence variants of the human IC47611 protein that maintain the ability to bind an IC47611 ligand or substrate and/or modulate cell proliferation and/or migration mechanisms. Functional allelic variants will typically contain only conservative substitution of one or more amino acids of SEQ ID NO:32, or substitution, deletion or insertion of non-critical residues in non-critical regions of the protein.

Non-functional allelic variants are naturally occurring amino acid sequence variants of the human IC47611 protein that do not have the ability to either bind an IC47611 ligand and/or modulate any of the IC47611 activities described herein. Non-functional allelic variants will typically contain a non-conservative substitution, a deletion, or insertion or premature truncation of the amino acid sequence of SEQ ID NO:32, or a substitution, insertion or deletion in critical residues or critical regions.

The present invention further provides non-human orthologues of the human IC47611 protein. Orthologues of the human IC47611 protein are proteins that are isolated from non-human organisms and possess the same IC47611 ligand binding and/or modulation of membrane excitability activities of the human IC47611 protein. Orthologues of the human IC47611 protein can readily be identified as comprising an amino acid sequence that is substantially identical to SEQ ID NO:32.

Moreover, nucleic acid molecules encoding other IC47611 family members and, thus, which have a nucleotide sequence which differs from the IC47611 sequences of SEQ ID NO:31 or 33, or the nucleotide sequence of the DNA insert of the plasmid deposited with ATCC as Accession Number ______ are intended to be within the scope of the invention. For example, another IC47611 cDNA can be identified based on the nucleotide sequence of human IC47611. Moreover, nucleic acid molecules encoding IC47611 proteins from different species, and which, thus, have a nucleotide sequence which differs from the IC47611 sequences of SEQ ID NO:31 or 33, or the nucleotide sequence of the DNA insert of the plasmid deposited with ATCC as Accession Number ______ are intended to be within the scope of the invention. For example, a mouse IC47611 cDNA can be identified based on the nucleotide sequence of a human IC47611.

Nucleic acid molecules corresponding to natural allelic variants and homologues of the IC47611 cDNAs of the invention can be isolated based on their homology to the IC47611 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the IC47611 cDNAs of the invention can further be isolated by mapping to the same chromosome or locus as the IC47611 gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:31 or 33, or the nucleotide sequence of the DNA insert of the plasmid deposited with ATCC as Accession Number ______. In other embodiment, the nucleic acid is at least 50-100, 100-150, 150-200, 200-216, 216-250, 250-300, 300-340, 340-350, 350-400, 400-438, 438-450, 450-500, 500-528, 528-542, 542-550, 550-600, 600-650, 650-700, 700-750, 750-764, 764-800, 800-841, 841-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1950-2000, 2000-2050, 2050-2100, 2100-2150, 2150-2200, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4×sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

Preferably, an isolated nucleic acid molecule of the invention that hybridizes under stringent conditions to the sequence of SEQ ID NO:31 or 33, and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the IC47611 sequences that may exist in the population, the skilled artisan will further appreciate that changes can be introduced by mutation into the nucleotide sequences of SEQ ID NO:31 or 33, or the nucleotide sequence of the DNA insert of the plasmid deposited with ATCC as Accession Number ______, thereby leading to changes in the amino acid sequence of the encoded IC47611 proteins, without altering the functional ability of the IC47611 proteins. For example, nucleotide substitutions leading to amino acid substitutions at “non-essential” amino acid residues can be made in the sequence of SEQ ID NO:31 or 33, or the nucleotide sequence of the DNA insert of the plasmid deposited with ATCC as Accession Number ______. A “non-essential” amino acid residue is a residue that can be altered from the wild-type sequence of IC47611 (e.g., the sequence of SEQ ID NO:32) without altering the biological activity, whereas an “essential” amino acid residue is required for biological activity. For example, amino acid residues that are conserved among the IC47611 proteins of the present invention, e.g., those present in a transmembrane domain, are predicted to be particularly unamenable to alteration. Furthermore, additional amino acid residues that are conserved between the IC47611 proteins of the present invention and other members of the IC47611 family are not likely to be amenable to alteration.

Accordingly, another aspect of the invention pertains to nucleic acid molecules encoding IC47611 proteins that contain changes in amino acid residues that are not essential for activity. Such IC47611 proteins differ in amino acid sequence from SEQ ID NO:32, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to SEQ ID NO:32.

An isolated nucleic acid molecule encoding an IC47611 protein identical to the protein of SEQ ID NO:32, can be created by introducing one or more nucleotide substitutions, additions or deletions into the nucleotide sequence of SEQ ID NO:31 or 33, or the nucleotide sequence of the DNA insert of the plasmid deposited with ATCC as Accession Number ______, such that one or more amino acid substitutions, additions or deletions are introduced into the encoded protein. Mutations can be introduced into SEQ ID NO:31 or 33, or the nucleotide sequence of the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an IC47611 protein is preferably replaced with another amino acid residue from the same side chain family. Alternatively, in another embodiment, mutations can be introduced randomly along all or part of an IC47611 coding sequence, such as by saturation mutagenesis, and the resultant mutants can be screened for IC47611 biological activity to identify mutants that retain activity. Following mutagenesis of SEQ ID NO:31 or 33, or the nucleotide sequence of the DNA insert of the plasmid deposited with ATCC as Accession Number ______, the encoded protein can be expressed recombinantly and the activity of the protein can be determined.

In a preferred embodiment, a mutant IC47611 protein can be assayed for the ability to (1) modulate membrane excitability, (2) regulate intracellular ion concentration, (3) modulate membrane polarization (e.g., membrane polarization and/or depolarization), (4) regulate action potential, (5) regulate cellular signal transduction, (6) regulate neurotransmitter release (e.g., from neuronal cells), (7) modulate synaptic transmission, (8) regulate neuronal excitability and/or plasticity, (9) regulate muscle contraction, (10) regulate cell activation and (11) regulate cellular proliferation, growth, migration and/or differentiation.

In addition to the nucleic acid molecules encoding IC47611 proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire IC47611 coding strand, or to only a portion thereof. In one embodiment, an antisense nucleic acid molecule is antisense to a “coding region” of the coding strand of a nucleotide sequence encoding IC47611. The term “coding region” refers to the region of the nucleotide sequence comprising codons which are translated into amino acid residues (e.g., the coding region of human IC47611 corresponds to SEQ ID NO:33). In another embodiment, the antisense nucleic acid molecule is antisense to a “noncoding region” of the coding strand of a nucleotide sequence encoding IC47611. The term “noncoding region” refers to 5′ and 3′ sequences which flank the coding region that are not translated into amino acids (i.e., also referred to as 5′ and 3′ untranslated regions).

Given the coding strand sequences encoding IC47611 disclosed herein (e.g., SEQ ID NO:33), antisense nucleic acids of the invention can be designed according to the rules of Watson and Crick base pairing. The antisense nucleic acid molecule can be complementary to the entire coding region of IC47611 mRNA, but more preferably is an oligonucleotide which is antisense to only a portion of the coding or noncoding region of IC47611 mRNA. For example, the antisense oligonucleotide can be complementary to the region surrounding the translation start site of IC47611 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an IC47611 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave IC47611 mRNA transcripts to thereby inhibit translation of IC47611 mRNA. A ribozyme having specificity for an IC47611-encoding nucleic acid can be designed based upon the nucleotide sequence of an IC47611 cDNA disclosed herein (i.e., SEQ ID NO:31 or 33, or the nucleotide sequence of the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in an IC47611-encoding mRNA. See, e.g., Cech et al. U.S. Pat. No. 4,987,071; and Cech et al. U.S. Pat. No. 5,116,742. Alternatively, IC47611 mRNA can be used to select a catalytic RNA having a specific ribonuclease activity from a pool of RNA molecules. See, e.g., Bartel, D. and Szostak, J. W. (1993) Science 261:1411-1418.

Alternatively, IC47611 gene expression can be inhibited by targeting nucleotide sequences complementary to the regulatory region of the IC47611 (e.g., the IC47611 promoter and/or enhancers; e.g., nucleotides 1-366 of SEQ ID NO:31) to form triple helical structures that prevent transcription of the IC47611 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the IC47611 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad Sci. 93: 14670-675.

PNAs of IC47611 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of IC47611 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of IC47611 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of IC47611 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous IC47611 gene within a cell line or microorganism may be modified by inserting a heterologous DNA regulatory element into the genome of a stable cell line or cloned microorganism such that the inserted regulatory element is operatively linked with the endogenous IC47611 gene. For example, an endogenous IC47611 gene which is normally “transcriptionally silent”, i.e., an IC47611 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous IC47611 gene may be activated by insertion of a promiscuous regulatory element that works across cell types.

A heterologous regulatory element may be inserted into a stable cell line or cloned microorganism, such that it is operatively linked with an endogenous IC47611 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated IC47611 Proteins and Anti-IC47611 Antibodies

One aspect of the invention pertains to isolated IC47611 proteins, and biologically active portions thereof, as well as polypeptide fragments suitable for use as immunogens to raise anti-IC47611 antibodies. In one embodiment, native IC47611 proteins can be isolated from cells or tissue sources by an appropriate purification scheme using standard protein purification techniques. In another embodiment, IC47611 proteins are produced by recombinant DNA techniques. Alternative to recombinant expression, an IC47611 protein or polypeptide can be synthesized chemically using standard peptide synthesis techniques.

An “isolated” or “purified” protein or biologically active portion thereof is substantially free of cellular material or other contaminating proteins from the cell or tissue source from which the IC47611 protein is derived, or substantially free from chemical precursors or other chemicals when chemically synthesized. The language “substantially free of cellular material” includes preparations of IC47611 protein in which the protein is separated from cellular components of the cells from which it is isolated or recombinantly produced. In one embodiment, the language “substantially free of cellular material” includes preparations of IC47611 protein having less than about 30% (by dry weight) of non-IC47611 protein (also referred to herein as a “contaminating protein”), more preferably less than about 20% of non-IC47611 protein, still more preferably less than about 10% of non-IC47611 protein, and most preferably less than about 5% non-IC47611 protein. When the IC47611 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of IC47611 protein in which the protein is separated from chemical precursors or other chemicals which are involved in the synthesis of the protein. In one embodiment, the language “substantially free of chemical precursors or other chemicals” includes preparations of IC47611 protein having less than about 30% (by dry weight) of chemical precursors or non-IC47611 chemicals, more preferably less than about 20% chemical precursors or non-IC47611 chemicals, still more preferably less than about 10% chemical precursors or non-IC47611 chemicals, and most preferably less than about 5% chemical precursors or non-IC47611 chemicals.

As used herein, a “biologically active portion” of an IC47611 protein includes a fragment of an IC47611 protein which participates in an interaction between an IC47611 molecule and a non-IC47611 molecule. Biologically active portions of an IC47611 protein include peptides comprising amino acid sequences sufficiently identical to or derived from the amino acid sequence of the IC47611 protein, e.g., the amino acid sequence shown in SEQ ID NO:32, which include less amino acids than the full length IC47611 proteins, and exhibit at least one activity of an IC47611 protein. Typically, biologically active portions comprise a domain or motif with at least one activity of the IC47611 protein, e.g., modulating membrane excitability. A biologically active portion of an IC47611 protein can be a polypeptide which is, for example, 25, 50, 75, 100, 125, 150, 175, 200 or more amino acids in length. Biologically active portions of an IC47611 protein can be used as targets for developing agents which modulate an IC47611 mediated activity, e.g., modulation of membrane excitability.

In one embodiment, a biologically active portion of an IC47611 protein comprises at least one transmembrane domain. It is to be understood that a preferred biologically active portion of an IC47611 protein of the present invention may contain one or more of the following domains: a transmembrane domain, an inward-rectifier potassium channel (IRK) domain, and an inward-rectifier potassium channel (IRK)-related domain. Moreover, other biologically active portions, in which other regions of the protein are deleted, can be prepared by recombinant techniques and evaluated for one or more of the functional activities of a native IC47611 protein.

In a preferred embodiment, the IC47611 protein has an amino acid sequence shown in SEQ ID NO:32. In other embodiments, the IC47611 protein is substantially identical to SEQ ID NO:32, and retains the functional activity of the protein of SEQ ID NO:32, yet differs in amino acid sequence due to natural allelic variation or mutagenesis, as described in detail in subsection I above. Accordingly, in another embodiment, the IC47611 protein is a protein which comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to SEQ ID NO:32.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the IC47611 amino acid sequence of SEQ ID NO:32 having 453 amino acid residues, at least 136, preferably at least 181, more preferably at least 227, even more preferably at least 272, and even more preferably at least 317, 362, 408 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to IC47611 nucleic acid molecules of the invention. BLAST protein searches can be performed with the XBLAST program, score=100, wordlength=3 to obtain amino acid sequences homologous to IC47611 protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See http://www.ncbi.nlm.nih.gov.

The invention also provides IC47611 chimeric or fusion proteins. As used herein, an IC47611 “chimeric protein” or “fusion protein” comprises an IC47611 polypeptide operatively linked to a non-IC47611 polypeptide. An “IC47611 polypeptide” refers to a polypeptide having an amino acid sequence corresponding to IC47611, whereas a “non-IC47611 polypeptide” refers to a polypeptide having an amino acid sequence corresponding to a protein which is not substantially homologous to the IC47611 protein, e.g., a protein which is different from the IC47611 protein and which is derived from the same or a different organism. Within an IC47611 fusion protein the IC47611 polypeptide can correspond to all or a portion of an IC47611 protein. In a preferred embodiment, an IC47611 fusion protein comprises at least one biologically active portion of an IC47611 protein. In another preferred embodiment, an IC47611 fusion protein comprises at least two biologically active portions of an IC47611 protein. Within the fusion protein, the term “operatively linked” is intended to indicate that the IC47611 polypeptide and the non-IC47611 polypeptide are fused in-frame to each other. The non-IC47611 polypeptide can be fused to the N-terminus or C-terminus of the IC47611 polypeptide.

For example, in one embodiment, the fusion protein is a GST-IC47611 fusion protein in which the IC47611 sequences are fused to the C-terminus of the GST sequences. Such fusion proteins can facilitate the purification of recombinant IC47611.

In another embodiment, the fusion protein is an IC47611 protein containing a heterologous signal sequence at its N-terminus. In certain host cells (e.g., mammalian host cells), expression and/or secretion of IC47611 can be increased through use of a beterologous signal sequence.

The IC47611 fusion proteins of the invention can be incorporated into pharmaceutical compositions and administered to a subject in vivo. The IC47611 fusion proteins can be used to affect the bioavailability of an IC47611 substrate. Use of IC47611 fusion proteins may be useful therapeutically for the treatment of disorders caused by, for example, (i) aberrant modification or mutation of a gene encoding an IC47611 protein; (ii) mis-regulation of the IC47611 gene; and (iii) aberrant post-translational modification of an IC47611 protein.

Moreover, the IC47611-fusion proteins of the invention can be used as immunogens to produce anti-IC47611 antibodies in a subject, to purify IC47611 ligands and in screening assays to identify molecules which inhibit the interaction of IC47611 with an IC47611 substrate.

Preferably, an IC47611 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An IC47611-encoding nucleic acid can be cloned into such an expression vector such that the fusion moiety is linked in-frame to the IC47611 protein.

The present invention also pertains to variants of the IC47611 proteins which function as either IC47611 agonists (mimetics) or as IC47611 antagonists. Variants of the IC47611 proteins can be generated by mutagenesis, e.g., discrete point mutation or truncation of an IC47611 protein. An agonist of the IC47611 proteins can retain substantially the same, or a subset, of the biological activities of the naturally occurring form of an IC47611 protein. An antagonist of an IC47611 protein can inhibit one or more of the activities of the naturally occurring form of the IC47611 protein by, for example, competitively modulating an IC47611-mediated activity of an IC47611 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the IC47611 protein.

In one embodiment, variants of an IC47611 protein which function as either IC47611 agonists (mimetics) or as IC47611 antagonists can be identified by screening combinatorial libraries of mutants, e.g., truncation mutants, of an IC47611 protein for IC47611 protein agonist or antagonist activity. In one embodiment, a variegated library of IC47611 variants is generated by combinatorial mutagenesis at the nucleic acid level and is encoded by a variegated gene library. A variegated library of IC47611 variants can be produced by, for example, enzymatically ligating a mixture of synthetic oligonucleotides into gene sequences such that a degenerate set of potential IC47611 sequences is expressible as individual polypeptides, or alternatively, as a set of larger fusion proteins (e.g., for phage display) containing the set of IC47611 sequences therein. There are a variety of methods that can be used to produce libraries of potential IC47611 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential IC47611 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of an IC47611 protein coding sequence can be used to generate a variegated population of IC47611 fragments for screening and subsequent selection of variants of an IC47611 protein. In one embodiment, a library of coding sequence fragments can be generated by treating a double stranded PCR fragment of an IC47611 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the IC47611 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of IC47611 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify IC47611 variants (Arkin and Youvan (1992) Proc. Natl. Acad. Sci. USA 89:7811-7815; Delagrave et al. (1993) Protein Engineering 6(3): 327-331).

In one embodiment, cell based assays can be exploited to analyze a variegated IC47611 library. For example, a library of expression vectors can be transfected into a cell line, e.g., a neuronal cell line, which ordinarily responds to an IC47611 ligand in a particular IC47611 ligand-dependent manner. The transfected cells are then contacted with an IC47611 ligand and the effect of expression of the mutant on, e.g., membrane excitability of IC47611 can be detected. Plasmid DNA can then be recovered from the cells which score for inhibition, or alternatively, potentiation of signaling by the IC47611 ligand, and the individual clones further characterized.

An isolated IC47611 protein, or a portion or fragment thereof, can be used as an immunogen to generate antibodies that bind IC47611 using standard techniques for polyclonal and monoclonal antibody preparation. A full-length IC47611 protein can be used or, alternatively, the invention provides antigenic peptide fragments of IC47611 for use as immunogens. The antigenic peptide of IC47611 comprises at least 8 amino acid residues of the amino acid sequence shown in SEQ ID NO:32 and encompasses an epitope of IC47611 such that an antibody raised against the peptide forms a specific immune complex with IC47611. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of IC47611 that are located on the surface of the protein, e.g., hydrophilic regions, as well as regions with high antigenicity (see FIG. 25).

An IC47611 immunogen typically is used to prepare antibodies by immunizing a suitable subject, (e.g., rabbit, goat, mouse or other mammal) with the immunogen. An appropriate immunogenic preparation can contain, for example, recombinantly expressed IC47611 protein or a chemically synthesized IC47611 polypeptide. The preparation can further include an adjuvant, such as Freund's complete or incomplete adjuvant, or similar immunostimulatory agent. Immunization of a suitable subject with an immunogenic IC47611 preparation induces a polyclonal anti-IC47611 antibody response.

Accordingly, another aspect of the invention pertains to anti-IC47611 antibodies. The term “antibody” as used herein refers to immunoglobulin molecules and immunologically active portions of immunoglobulin molecules, i.e., molecules that contain an antigen binding site which specifically binds (immunoreacts with) an antigen, such as IC47611. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind IC47611. The term “monoclonal antibody” or “monoclonal antibody composition”, as used herein, refers to a population of antibody molecules that contain only one species of an antigen binding site capable of immunoreacting with a particular epitope of IC47611. A monoclonal antibody composition thus typically displays a single binding affinity for a particular IC47611 protein with which it immunoreacts.

Polyclonal anti-IC47611 antibodies can be prepared as described above by immunizing a suitable subject with an IC47611 immunogen. The anti-IC47611 antibody titer in the immunized subject can be monitored over time by standard techniques, such as with an enzyme linked immunosorbent assay (ELISA) using immobilized IC47611. If desired, the antibody molecules directed against IC47611 can be isolated from the mammal (e.g., from the blood) and further purified by well known techniques, such as protein A chromatography to obtain the IgG fraction. At an appropriate time after immunization, e.g., when the anti-IC47611 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N. Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an IC47611 immunogen as described above, and the culture supernatants of the resulting hybridoma cells are screened to identify a hybridoma producing a monoclonal antibody that binds IC47611.

Any of the many well known protocols used for fusing lymphocytes and immortalized cell lines can be applied for the purpose of generating an anti-IC47611 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind IC47611, e.g., using a standard ELISA assay.

Alternative to preparing monoclonal antibody-secreting hybridomas, a monoclonal anti-IC47611 antibody can be identified and isolated by screening a recombinant combinatorial immunoglobulin library (e.g., an antibody phage display library) with IC47611 to thereby isolate immunoglobulin library members that bind IC47611. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-IC47611 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-IC47611 antibody (e.g., monoclonal antibody) can be used to isolate IC47611 by standard techniques, such as affinity chromatography or immunoprecipitation. An anti-IC47611 antibody can facilitate the purification of natural IC47611 from cells and of recombinantly produced IC47611 expressed in host cells. Moreover, an anti-IC47611 antibody can be used to detect IC47611 protein (e.g., in a cellular lysate or cell supernatant) in order to evaluate the abundance and pattern of expression of the IC47611 protein. Anti-IC47611 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding an IC47611 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., IC47611 proteins, mutant forms of IC47611 proteins, fusion proteins, and the like).

The recombinant expression vectors of the invention can be designed for expression of IC47611 proteins in prokaryotic or eukaryotic cells. For example, IC47611 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in IC47611 activity assays, (e.g., direct assays or competitive assays described in detail below), or to generate antibodies specific for IC47611 proteins, for example. In a preferred embodiment, an IC47611 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the IC47611 expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp, San Diego, Calif.).

Alternatively, IC47611 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to IC47611 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which an IC47611 nucleic acid molecule of the invention is introduced, e.g., an IC47611 nucleic acid molecule within a recombinant expression vector or an IC47611 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an IC47611 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an IC47611 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an IC47611 protein. Accordingly, the invention further provides methods for producing an IC47611 protein using the host cells of the invention. In one embodiment, the method comprises culturing the host cell of the invention (into which a recombinant expression vector encoding an IC47611 protein has been introduced) in a suitable medium such that an IC47611 protein is produced. In another embodiment, the method further comprises isolating an IC47611 protein from the medium or the host cell.

The host cells of the invention can also be used to produce non-human transgenic animals. For example, in one embodiment, a host cell of the invention is a fertilized oocyte or an embryonic stem cell into which IC47611-coding sequences have been introduced. Such host cells can then be used to create non-human transgenic animals in which exogenous IC47611 sequences have been introduced into their genome or homologous recombinant animals in which endogenous IC47611 sequences have been altered. Such animals are useful for studying the function and/or activity of an IC47611 and for identifying and/or evaluating modulators of IC47611 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous IC47611 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an IC47611-encoding nucleic acid into the male pronuclei of a fertilized oocyte, e.g., by microinjection, retroviral infection, and allowing the oocyte to develop in a pseudopregnant female foster animal. The IC47611 cDNA sequence of SEQ ID NO:31 can be introduced as a transgene into the genome of a non-human animal. Alternatively, a nonhuman homologue of a human IC47611 gene, such as a mouse or rat IC47611 gene, can be used as a transgene. Alternatively, an IC47611 gene homologue, such as another IC47611 family member, can be isolated based on hybridization to the IC47611 cDNA sequences of SEQ ID NO:31 or 33, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to an IC47611 transgene to direct expression of an IC47611 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of an IC47611 transgene in its genome and/or expression of IC47611 mRNA in tissues or cells of the animals. A transgenic founder animal can then be used to breed additional animals carrying the transgene. Moreover, transgenic animals carrying a transgene encoding an IC47611 protein can further be bred to other transgenic animals carrying other transgenes.

To create a homologous recombinant animal, a vector is prepared which contains at least a portion of an IC47611 gene into which a deletion, addition or substitution has been introduced to thereby alter, e.g., functionally disrupt, the IC47611 gene. The IC47611 gene can be a human gene (e.g., the cDNA of SEQ ID NO:33), but more preferably, is a non-human homologue of a human IC47611 gene (e.g., a cDNA isolated by stringent hybridization with the nucleotide sequence of SEQ ID NO:31). For example, a mouse IC47611 gene can be used to construct a homologous recombination nucleic acid molecule, e.g., a vector, suitable for altering an endogenous IC47611 gene in the mouse genome. In a preferred embodiment, the homologous recombination nucleic acid molecule is designed such that, upon homologous recombination, the endogenous IC47611 gene is functionally disrupted (i.e., no longer encodes a functional protein; also referred to as a “knock out” vector). Alternatively, the homologous recombination nucleic acid molecule can be designed such that, upon homologous recombination, the endogenous IC47611 gene is mutated or otherwise altered but still encodes functional protein (e.g., the upstream regulatory region can be altered to thereby alter the expression of the endogenous IC47611 protein). In the homologous recombination nucleic acid molecule, the altered portion of the IC47611 gene is flanked at its 5′ and 3′ ends by additional nucleic acid sequence of the IC47611 gene to allow for homologous recombination to occur between the exogenous IC47611 gene carried by the homologous recombination nucleic acid molecule and an endogenous IC47611 gene in a cell, e.g., an embryonic stem cell. The additional flanking IC47611 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced IC47611 gene has homologously recombined with the endogenous IC47611 gene are selected (see e.g., Li, E. et al. (1992) Cell 69:915). The selected cells can then be injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The IC47611 nucleic acid molecules, fragments of IC47611 proteins, and anti-IC47611 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g, a fragment of an IC47611 protein or an anti-IC47611 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g, Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, an IC47611 protein of the invention has one or more of the following activities: (1) modulates membrane excitability, (2) regulates intracellular ion concentration, (3) modulates membrane polarization (e.g., membrane polarization and/or depolarization), (4) regulates action potential, (5) regulates cellular signal transduction, (6) regulates neurotransmitter release (e.g., from neuronal cells), (7) modulates synaptic transmission, (8) regulates neuronal excitability and/or plasticity, (9) regulates muscle contraction, (10) regulate cell activation and (11) regulates cellular proliferation, growth, migration and/or differentiation.

The isolated nucleic acid molecules of the invention can be used, for example, to express IC47611 protein (e.g., via a recombinant expression vector in a host cell in gene therapy applications), to detect IC47611 mRNA (e.g., in a biological sample) or a genetic alteration in an IC47611 gene, and to modulate IC47611 activity, as described further below. The IC47611 proteins can be used to treat disorders characterized by insufficient or excessive production of an IC47611 substrate or production of IC47611 inhibitors. In addition, the IC47611 proteins can be used to screen for naturally occurring IC47611 substrates, to screen for drugs or compounds which modulate IC47611 activity, as well as to treat disorders characterized by insufficient or excessive production of IC47611 protein or production of IC47611 protein forms which have decreased, aberrant or unwanted activity compared to IC47611 wild type protein (e.g., proliferative disorders, CNS disorders such as cognitive and neurodegenerative disorders (e.g., Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, and bipolar affective disorder (e.g., severe bipolar affective (mood) disorder (BP-1) and bipolar affective neurological disorders (e.g., migraine and obesity)); pain disorders; cardiac disorders (e.g., arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia), muscular disorders (e.g., paralysis, muscle weakness (e.g., ataxia, myotonia, and myokymia), muscular dystrophy (e.g., Duchenne muscular dystrophy or myotonic dystrophy), spinal muscular atrophy, congenital myopathies, central core disease, rod myopathy, central nuclear myopathy, Lambert-Eaton syndrome, denervation, and infantile spinal muscular atrophy (Werdnig-Hoffman disease), and cellular growth, differentiation, or migration disorders (e.g., cancer, e.g., carcinoma, sarcoma, or leukemia; tumor angiogenesis and metastasis; skeletal dysplasia; neuronal deficiencies resulting from impaired neural induction and patterning; hepatic disorders; cardiovascular disorders; and hematopoietic and/or myeloproliferative disorders). Moreover, the anti-IC47611 antibodies of the invention can be used to detect and isolate IC47611 proteins, regulate the bioavailability of IC47611 proteins, and modulate IC47611 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to IC47611 proteins, have a stimulatory or inhibitory effect on, for example, IC47611 expression or IC47611 activity, or have a stimulatory or inhibitory effect on, for example, the expression or activity of IC47611 substrate.

In one embodiment, the invention provides assays for screening candidate or test compounds which are substrates of an IC47611 protein or polypeptide or biologically active portion thereof. In another embodiment, the invention provides assays for screening candidate or test compounds which bind to or modulate the activity of an IC47611 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91: 11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an IC47611 protein or biologically active portion thereof is contacted with a test compound and the ability of the test compound to modulate IC47611 activity is determined. Determining the ability of the test compound to modulate IC47611 activity can be accomplished by monitoring, for example, the release of a neurotransmitter from a cell which expresses IC47611. The cell, for example, can be of mammalian origin, e.g., a neuronal cell or a thymus cell. The ability of the test compound to modulate IC47611 binding to a substrate or to bind to IC47611 can also be determined. Determining the ability of the test compound to modulate IC47611 binding to a substrate can be accomplished, for example, by coupling the IC47611 substrate with a radioisotope or enzymatic label such that binding of the IC47611 substrate to IC47611 can be determined by detecting the labeled IC47611 substrate in a complex. Alternatively, IC47611 could be coupled with a radioisotope or enzymatic label to monitor the ability of a test compound to modulate IC47611 binding to an IC47611 substrate in a complex. Determining the ability of the test compound to bind IC47611 can be accomplished, for example, by coupling the compound with a radioisotope or enzymatic label such that binding of the compound to IC47611 can be determined by detecting the labeled IC47611 compound in a complex. For example, compounds (e.g., IC47611 substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., an IC47611 substrate) to interact with IC47611 without the labeling of any of the interactants. For example, a microphysiometer can be used to detect the interaction of a compound with IC47611 without the labeling of either the compound or the IC47611. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and IC47611.

In another embodiment, an assay is a cell-based assay comprising contacting a cell expressing an IC47611 target molecule (e.g., an IC47611 substrate) with a test compound and determining the ability of the test compound to modulate (e.g., stimulate or inhibit) the activity of the IC47611 target molecule. Determining the ability of the test compound to modulate the activity of an IC47611 target molecule can be accomplished, for example, by determining the ability of the IC47611 protein to bind to or interact with the IC47611 target molecule.

Determining the ability of the IC47611 protein, or a biologically active fragment thereof, to bind to or interact with an IC47611 target molecule can be accomplished by one of the methods described above for determining direct binding. In a preferred embodiment, determining the ability of the IC47611 protein to bind to or interact with an IC47611 target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target using an appropriate substrate, detecting the induction of a reporter gene (comprising a target-responsive regulatory element operatively linked to a nucleic acid encoding a detectable marker, e.g., luciferase), or detecting a target regulated cellular response.

In yet another embodiment, an assay of the present invention is a cell-free assay in which an IC47611 protein or biologically active portion thereof is-contacted with a test compound and the ability of the test compound to bind to the IC47611 protein or biologically active portion thereof is determined. Preferred biologically active portions of the IC47611 proteins to be used in assays of the present invention include fragments which participate in interactions with non-IC47611 molecules, e.g., fragments with high surface probability scores (see, for example, FIG. 25). Binding of the test compound to the IC47611 protein can be determined either directly or indirectly as described above. In a preferred embodiment, the assay includes contacting the IC47611 protein or biologically active portion thereof with a known compound which binds IC47611 to form an assay mixture, contacting the assay mixture with a test compound, and determining the ability of the test compound to interact with an IC47611 protein, wherein determining the ability of the test compound to interact with an IC47611 protein comprises determining the ability of the test compound to preferentially bind to IC47611 or biologically active portion thereof as compared to the known compound.

In another embodiment, the assay is a cell-free assay in which an IC47611 protein or biologically active portion thereof is contacted with a test compound and the ability of the test compound to modulate (e.g., stimulate or inhibit) the activity of the IC47611 protein or biologically active portion thereof is determined. Determining the ability of the test compound to modulate the activity of an IC47611 protein can be accomplished, for example, by determining the ability of the IC47611 protein to bind to an IC47611 target molecule by one of the methods described above for determining direct binding. Determining the ability of the IC47611 protein to bind to an IC47611 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an IC47611 protein can be accomplished by determining the ability of the IC47611 protein to further modulate the activity of a downstream effector of an IC47611 target molecule. For example, the activity of the effector molecule on an appropriate target can be determined or the binding of the effector to an appropriate target can be determined as previously described.

In yet another embodiment, the cell-free assay involves contacting an IC47611 protein or biologically active portion thereof with a known compound which binds the IC47611 protein to form an assay mixture, contacting the assay mixture with a test compound, and determining the ability of the test compound to interact with the IC47611 protein, wherein determining the ability of the test compound to interact with the IC47611 protein comprises determining the ability of the IC47611protein to preferentially bind to or modulate the activity of an IC47611 target molecule.

In more than one embodiment of the above assay methods of the present invention, it may be desirable to immobilize either IC47611 or its target molecule to facilitate separation of complexed from uncomplexed forms of one or both of the proteins, as well as to accommodate automation of the assay. Binding of a test compound to an IC47611 protein, or interaction of an IC47611 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/IC47611 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or IC47611 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of IC47611 binding or activity determined using standard techniques.

Other techniques for immobilizing proteins on matrices can also be used in the screening assays of the invention. For example, either an IC47611 protein or an IC47611 target molecule can be immobilized utilizing conjugation of biotin and streptavidin. Biotinylated IC47611 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with IC47611 protein or target molecules but which do not interfere with binding of the IC47611 protein to its target molecule can be derivatized to the wells of the plate, and unbound target or IC47611 protein trapped in the wells by antibody conjugation. Methods for detecting such complexes, in addition to those described above for the GST-immobilized complexes, include immunodetection of complexes using antibodies reactive with the IC47611 protein or target molecule, as well as enzyme-linked assays which rely on detecting an enzymatic activity associated with the IC47611 protein or target molecule.

In another embodiment, modulators of IC47611 expression are identified in a method wherein a cell is contacted with a candidate compound and the expression of IC47611 mRNA or protein in the cell is determined. The level of expression of IC47611 mRNA or protein in the presence of the candidate compound is compared to the level of expression of IC47611 mRNA or protein in the absence of the candidate compound. The candidate compound can then be identified as a modulator of IC47611 expression based on this comparison. For example, when expression of IC47611 mRNA or protein is greater (statistically significantly greater) in the presence of the candidate compound than in its absence, the candidate compound is identified as a stimulator of IC47611 mRNA or protein expression. Alternatively, when expression of IC47611 mRNA or protein is less (statistically significantly less) in the presence of the candidate compound than in its absence, the candidate compound is identified as an inhibitor of IC47611 mRNA or protein expression. The level of IC47611 mRNA or protein expression in the cells can be determined by methods described herein for detecting IC47611 mRNA or protein.

In yet another aspect of the invention, the IC47611 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with IC47611 (“IC47611-binding proteins” or “IC47611-bp”) and are involved in IC47611 activity. Such IC47611-binding proteins are also likely to be involved in the propagation of signals by the IC47611 proteins or IC47611 targets as, for example, downstream elements of an IC47611-mediated signaling pathway. Alternatively, such IC47611-binding proteins are likely to be IC47611 inhibitors.

The two-hybrid system is based on the modular nature of most transcription factors, which consist of separable DNA-binding and activation domains. Briefly, the assay utilizes two different DNA constructs. In one construct, the gene that codes for an IC47611 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an IC47611-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the IC47611 protein.

In another aspect, the invention pertains to a combination of two or more of the assays described herein. For example, a modulating agent can be identified using a cell-based or a cell free assay, and the ability of the agent to modulate the activity of an IC47611 protein can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., an IC47611 modulating agent, an antisense IC47611 nucleic acid molecule, an IC47611-specific antibody, or an IC47611-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the IC47611 nucleotide sequences, described herein, can be used to map the location of the IC47611 genes on a chromosome. The mapping of the IC47611 sequences to chromosomes is an important first step in correlating these sequences with genes associated with disease.

Briefly, IC47611 genes can be mapped to chromosomes by preparing PCR primers (preferably 15-25 bp in length) from the IC47611 nucleotide sequences. Computer analysis of the IC47611 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the IC47611 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the IC47611 nucleotide sequences to design oligonucleotide primers, sublocalization can be achieved with panels of fragments from specific chromosomes. Other mapping strategies which can similarly be used to map an IC47611 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the IC47611 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The IC47611 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot -to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the IC47611 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The IC47611 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:31 can comfortably provide positive individual identification with a panel of perhaps 10 to 1,000 primers which each yield a noncoding amplified sequence of 100 bases. If predicted coding sequences, such as that in SEQ ID NO:33 are used, a more appropriate number of primers for positive individual identification would be 500-2,000.

If a panel of reagents from IC47611 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of IC47611 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:31 are particularly appropriate for this use as greater numbers of polymorphisms occur in the noncoding regions, making it easier to differentiate individuals using this technique. Examples of polynucleotide reagents include the IC47611 nucleotide sequences or portions thereof, e.g., fragments derived from the noncoding regions of SEQ ID NO:31 having a length of at least bases, preferably at least 30 bases.

The IC47611 nucleotide sequences described herein can further be used to provide polynucleotide reagents, e.g., labeled or labelable probes which can be used in, for example, an in situ hybridization technique, to identify a specific tissue, e.g., thymus or brain tissue. This can be very useful in cases where a forensic pathologist is presented with a tissue of unknown origin. Panels of such IC47611 probes can be used to identify tissue by species and/or by organ type.

In a similar fashion, these reagents, e.g., IC47611 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining IC47611 protein and/or nucleic acid expression as well as IC47611 activity, in the context of a biological sample (e.g., blood, serum, cells, tissue) to thereby determine whether an individual is afflicted with a disease or disorder, or is at risk of developing a disorder, associated with aberrant or unwanted IC47611 expression or activity. The invention also provides for prognostic (or predictive) assays for determining whether an individual is at risk of developing a disorder associated with IC47611 protein, nucleic acid expression or activity. For example, mutations in an IC47611 gene can be assayed in a biological sample. Such assays can be used for prognostic or predictive purpose to thereby prophylactically treat an individual prior to the onset of a disorder characterized by or associated with IC47611 protein, nucleic acid expression or activity.

Another aspect of the invention pertains to monitoring the influence of agents (e.g., drugs, compounds) on the expression or activity of IC47611 in clinical trials.

These and other agents are described in further detail in the following sections.

1. Diagnostic Assays

An exemplary method for detecting the presence or absence of IC47611 protein or nucleic acid in a biological sample involves obtaining a biological sample from a test subject and contacting the biological sample with a compound or an agent capable of detecting IC47611 protein or nucleic acid (e.g., mRNA, or genomic DNA) that encodes IC47611 protein such that the presence of IC47611 protein or nucleic acid is detected in the biological sample. A preferred agent for detecting IC47611 mRNA or genomic DNA is a labeled nucleic acid probe capable of hybridizing to IC47611 mRNA or genomic DNA. The nucleic acid probe can be, for example, the IC47611 nucleic acid set forth in SEQ ID NO:31 or 33, or the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to IC47611 mRNA or genomic DNA. Other suitable probes for use in the diagnostic assays of the invention are described herein.

A preferred agent for detecting IC47611 protein is an antibody capable of binding to IC47611 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect IC47611 mRNA, protein, or genomic DNA in a biological sample in vitro as well as in vivo. For example, in vitro techniques for detection of IC47611 mRNA include Northern hybridizations and in situ hybridizations. In vitro techniques for detection of IC47611 protein include enzyme linked immunosorbent assays (ELISAs), Western blots, immunoprecipitations and immunofluorescence. In vitro techniques for detection of IC47611 genomic DNA include Southern hybridizations. Furthermore, in vivo techniques for detection of IC47611 protein include introducing into a subject a labeled anti-IC47611 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting IC47611 protein, mRNA, or genomic DNA, such that the presence of IC47611 protein, mRNA or genomic DNA is detected in the biological sample, and comparing the presence of IC47611 protein, mRNA or genomic DNA in the control sample with the presence of IC47611 protein, mRNA or genomic DNA in the test sample.

The invention also encompasses kits for detecting the presence of IC47611 in a biological sample. For example, the kit can comprise a labeled compound or agent capable of detecting IC47611 protein or mRNA in a biological sample; means for determining the amount of IC47611 in the sample; and means for comparing the amount of IC47611 in the sample with a standard. The compound or agent can be packaged in a suitable container. The kit can further comprise instructions for using the kit to detect IC47611 protein or nucleic acid.

2. Prognostic Assays

The diagnostic methods described herein can furthermore be utilized to identify subjects having or at risk of developing a disease or disorder associated with aberrant or unwanted IC47611 expression or activity. As used herein, the term “aberrant” includes an IC47611 expression or activity which deviates from the wild type IC47611 expression or activity. Aberrant expression or activity includes increased or decreased expression or activity, as well as expression or activity which does not follow the wild type developmental pattern of expression or the subcellular pattern of expression. For example, aberrant IC47611 expression or activity is intended to include the cases in which a mutation in the IC47611 gene causes the IC47611 gene to be under-expressed or over-expressed and situations in which such mutations result in a non-functional IC47611 protein or a protein which does not function in a wild-type fashion, e.g., a protein which does not interact with an IC47611 substrate, e.g., a non-IC47611 channel subunit or ligand, or one which interacts with a non-IC47611 substrate, e.g. a non-IC47611 channel subunit or ligand. As used herein, the term “unwanted” includes an unwanted phenomenon involved in a biological response such as cellular proliferation. For example, the term unwanted includes an IC47611 expression or activity which is undesirable in a subject.

The assays described herein, such as the preceding diagnostic assays or the following assays, can be utilized to identify a subject having or at risk of developing a disorder associated with a misregulation in IC47611 protein activity or nucleic acid expression, such as a CNS disorder (e.g., a cognitive or neurodegenerative disorder), a pain disorder, a muscular disorder, a cellular proliferation, growth, differentiation, or migration disorder, or a cardiovascular disorder. Alternatively, the prognostic assays can be utilized to identify a subject having or at risk for developing a disorder associated with a misregulation in IC47611 protein activity or nucleic acid expression, such as a CNS disorder, a pain disorder, a cellular proliferation, growth, differentiation, or migration disorder, a muscular disorder, or a cardiovascular disorder. Thus, the present invention provides a method for identifying a disease or disorder associated with aberrant or unwanted IC47611 expression or activity in which a test sample is obtained from a subject and IC47611 protein or nucleic acid (e.g., mRNA or genomic DNA) is detected, wherein the presence of IC47611 protein or nucleic acid is diagnostic for a subject having or at risk of developing a disease or disorder associated with aberrant or unwanted IC47611 expression or activity. As used herein, a “test sample” refers to a biological sample obtained from a subject of interest. For example, a test sample can be a biological fluid (e.g., cerebrospinal fluid or serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted IC47611 expression or activity. For example, such methods can be used to determine whether a subject can be effectively treated with an agent for a CNS disorder, a pain disorder, a muscular disorder, a cardiovascular disorder, or a cellular proliferation, growth, differentiation, or migration disorder. Thus, the present invention provides methods for determining whether a subject can be effectively treated with an agent for a disorder associated with aberrant or unwanted IC47611 expression or activity in which a test sample is obtained and IC47611 protein or nucleic acid expression or activity is detected (e.g., wherein the abundance of IC47611 protein or nucleic acid expression or activity is diagnostic for a subject that can be administered the agent to treat a disorder associated with aberrant or unwanted IC47611 expression or activity).

The methods of the invention can also be used to detect genetic alterations in an IC47611 gene, thereby determining if a subject with the altered gene is at risk for a disorder characterized by misregulation in IC47611 protein activity or nucleic acid expression, such as a CNS disorder, a pain disorder, a cellular proliferation, growth, differentiation, or migration disorder, a muscular disorder, or cardiovascular disorder. In preferred embodiments, the methods include detecting, in a sample of cells from the subject, the presence or absence of a genetic alteration characterized by at least one of an alteration affecting the integrity of a gene encoding an IC47611-protein, or the mis-expression of the IC47611 gene. For example, such genetic alterations can be detected by ascertaining the existence of at least one of 1) a deletion of one or more nucleotides from an IC47611 gene; 2) an addition of one or more nucleotides to an IC47611 gene; 3) a substitution of one or more nucleotides of an IC47611 gene, 4) a chromosomal rearrangement of an IC47611 gene; 5) an alteration in the level of a messenger RNA transcript of an IC47611 gene, 6) aberrant modification of an IC47611 gene, such as of the methylation pattern of the genomic DNA, 7) the presence of a non-wild type splicing pattern of a messenger RNA transcript of an IC47611 gene, 8) a non-wild type level of an IC47611-protein, 9) allelic loss of an IC47611 gene, and 10) inappropriate post-translational modification of an IC47611-protein. As described herein, there are a large number of assays known in the art which can be used for detecting alterations in an IC47611 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the IC47611-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to an IC47611 gene under conditions such that hybridization and amplification of the IC47611-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an IC47611 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in IC47611 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in IC47611 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the IC47611 gene and detect mutations by comparing the sequence of the sample IC47611 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the IC47611 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type IC47611 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in IC47611 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an IC47611 sequence, e.g., a wild-type IC47611 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in IC47611 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control IC47611 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an IC47611 gene.

Furthermore, any cell type or tissue in which IC47611 is expressed may be utilized in the prognostic assays described herein.

3. Monitoring of Effects During Clinical Trials

Monitoring the influence of agents (e.g., drugs) on the expression or activity of an IC47611 protein (e.g., the modulation of cell proliferation and/or migration) can be applied not only in basic drug screening, but also in clinical trials. For example, the effectiveness of an agent determined by a screening assay as described herein to increase IC47611 gene expression, protein levels, or upregulate IC47611 activity, can be monitored in clinical trials of subjects exhibiting decreased IC47611 gene expression, protein levels, or downregulated IC47611 activity. Alternatively, the effectiveness of an agent determined by a screening assay to decrease IC47611 gene expression, protein levels, or downregulate IC47611 activity, can be monitored in clinical trials of subjects exhibiting increased IC47611 gene expression, protein levels, or upregulated IC47611 activity. In such clinical trials, the expression or activity of an IC47611 gene, and preferably, other genes that have been implicated in, for example, an IC47611-associated disorder can be used as a “read out” or markers of the phenotype of a particular cell.

For example, and not by way of limitation, genes, including IC47611, that are modulated in cells by treatment with an agent (e.g., compound, drug or small molecule) which modulates IC47611 activity (e.g., identified in a screening assay as described herein) can be identified. Thus, to study the effect of agents on IC47611-associated disorders (e.g., disorders characterized by deregulated cell proliferation and/or migration), for example, in a clinical trial, cells can be isolated and RNA prepared and analyzed for the levels of expression of IC47611 and other genes implicated in the IC47611-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of IC47611 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an IC47611 protein, mRNA, or genomic DNA in the preadministration sample; (iii) obtaining one or more post-administration samples from the subject; (iv) detecting the level of expression or activity of the IC47611 protein, mRNA, or genomic DNA in the post-administration samples; (v) comparing the level of expression or activity of the IC47611 protein, mRNA, or genomic DNA in the pre-administration sample with the IC47611 protein, mRNA, or genomic DNA in the post administration sample or samples; and (vi) altering the administration of the agent to the subject accordingly. For example, increased administration of the agent may be desirable to increase the expression or activity of IC47611 to higher levels than detected, i.e., to increase the effectiveness of the agent. Alternatively, decreased administration of the agent may be desirable to decrease expression or activity of IC47611 to lower levels than detected, i.e. to decrease the effectiveness of the agent. According to such an embodiment, IC47611 expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted IC47611 expression or activity, e.g. a CNS disorder, a pain disorder, a cellular proliferation, growth, differentiation, or migration disorder, a muscular disorder, or a cardiovascular disorder. As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose to cure, heal, alleviate, relieve, alter, remedy, ameliorate, improve, or affect the disease or disorder, the symptom of the disease or disorder, or the risk of (or susceptibility to) the disease or disorder. As used herein, a “therapeutic agent” includes, but is not limited to, small molecules, peptides, polypeptides, antibodies, ribozymes, and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the IC47611 molecules of the present invention or IC47611 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted IC47611 expression or activity, by administering to the subject an IC47611 or an agent which modulates IC47611 expression or at least one IC47611 activity. Subjects at risk for a disease which is caused or contributed to by aberrant or unwanted IC47611 expression or activity can be identified by, for example, any or a combination of diagnostic or prognostic assays as described herein. Administration of a prophylactic agent can occur prior to the manifestation of symptoms characteristic of the IC47611 aberrancy, such that a disease or disorder is prevented or, alternatively, delayed in its progression. Depending on the type of IC47611 aberrancy, for example, an IC47611, IC47611 agonist or IC47611 antagonist agent can be used for treating the subject. The appropriate agent can be determined based on screening assays described herein.

2. Therapeutic Methods

Another aspect of the invention pertains to methods of modulating IC47611 expression or activity for therapeutic purposes. Accordingly, in an exemplary embodiment, the modulatory method of the invention involves contacting a cell with an IC47611 or agent that modulates one or more of the activities of IC47611 protein activity associated with the cell. An agent that modulates IC47611 protein activity can be an agent as described herein, such as a nucleic acid or a protein, a naturally-occurring target molecule of an IC47611 protein (e.g., an IC47611 substrate), an IC47611 antibody, an IC47611 agonist or antagonist, a peptidomimetic of an IC47611 agonist or antagonist, or other small molecule. In one embodiment, the agent stimulates one or more IC47611 activities. Examples of such stimulatory agents include active IC47611 protein and a nucleic acid molecule encoding IC47611 that has been introduced into the cell. In another embodiment, the agent inhibits one or more IC47611 activities. Examples of such inhibitory agents include antisense IC47611 nucleic acid molecules, anti-IC47611 antibodies, and IC47611 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of an IC47611 protein or nucleic acid molecule. In one embodiment, the method involves administering an agent (e.g., an agent identified by a screening assay described herein), or combination of agents that modulates (e.g., upregulates or downregulates) IC47611 expression or activity. In another embodiment, the method involves administering an IC47611 protein or nucleic acid molecule as therapy to compensate for reduced, aberrant, or unwanted IC47611 expression or activity.

Stimulation of IC47611 activity is desirable in situations in which IC47611 is abnormally downregulated and/or in which increased IC47611 activity is likely to have a beneficial effect. Likewise, inhibition of IC47611 activity is desirable in situations in which IC47611 is abnormally upregulated and/or in which decreased IC47611 activity is likely to have a beneficial effect.

3. Pharmacogenomics

The IC47611 molecules of the present invention, as well as agents, or modulators which have a stimulatory or inhibitory effect on IC47611 activity (e.g., IC47611 gene expression) as identified by a screening assay described herein can be administered to individuals to treat (prophylactically or therapeutically) IC47611-associated disorders (e.g., proliferative disorders) associated with aberrant or unwanted IC47611 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an IC47611 molecule or IC47611 modulator as well as tailoring the dosage and/or therapeutic regimen of treatment with an IC47611 molecule or IC47611 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2): 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., an IC47611 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme is the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an IC47611 molecule or IC47611 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an IC47611 molecule or IC47611 modulator, such as a modulator identified by one of the exemplary screening assays described herein.

E. Electronic Apparatus Readable Media and Arrays

Electronic apparatus readable media comprising IC47611 sequence information is also provided. As used herein, “IC47611 sequence information” refers to any nucleotide and/or amino acid sequence information particular to the IC47611 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said IC47611 sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding, or containing data or information that can be read and accessed directly by an electronic apparatus. Such media can include, but are not limited to: magnetic storage media, such as floppy discs, hard disc storage medium, and magnetic tape; optical storage media such as compact discs; electronic storage media such as RAM, ROM, EPROM, EEPROM and the like; and general hard disks and hybrids of these categories such as magnetic/optical storage media. The medium is adapted or configured for having recorded thereon IC47611 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatuses; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the IC47611 sequence information. A variety of software programs and formats can be used to store the sequence information on the electronic apparatus readable medium. For example, the sequence information can be represented in a word processing text file, formatted in commercially-available software such as WordPerfect and Microsoft Word, represented in the form of an ASCII file, or stored in a database application, such as DB2, Sybase, Oracle, or the like, as well as in other forms. Any number of dataprocessor structuring formats (e.g., text file or database) may be employed in order to obtain or create a medium having recorded thereon the IC47611 sequence information.

By providing IC47611 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a IC47611 associated disease or disorder or a pre-disposition to a IC47611 associated disease or disorder, wherein the method comprises the steps of determining IC47611 sequence information associated with the subject and based on the IC47611 sequence information, determining whether the subject has a IC47611 associated disease or disorder or a pre-disposition to a IC47611 associated disease or disorder, and/or recommending a particular treatment for the disease, disorder, or pre-disease condition.

The present invention further provides in an electronic system and/or in a network, a method for determining whether a subject has a IC47611 associated disease or disorder or a pre-disposition to a disease associated with IC47611 wherein the method comprises the steps of determining IC47611 sequence information associated with the subject, and based on the IC47611 sequence information, determining whether the subject has a IC47611 associated disease or disorder or a pre-disposition to a IC47611 associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a IC47611 associated disease or disorder or a pre-disposition to a IC47611 associated disease or disorder associated with IC47611, said method comprising the steps of receiving IC47611 sequence information from the subject and/or information related thereto, receiving phenotypic information associated with the subject, acquiring information from the network corresponding to IC47611 and/or a IC47611 associated disease or disorder, and based on one or more of the phenotypic information, the IC47611 information (e.g., sequence information and/or information related thereto), and the acquired information, determining whether the subject has a IC47611 associated disease or disorder or a pre-disposition to a IC47611 associated disease or disorder. The method may further comprise the step of recommending a particular treatment for the disease, disorder or pre-disease condition.

The present invention also provides a business method for determining whether a subject has a IC47611 associated disease or disorder or a pre-disposition to a IC47611 associated disease or disorder, said method comprising the steps of receiving information related to IC47611 (e.g., sequence information and/or information related thereto), receiving phenotypic information associated with the subject, acquiring information from the network related to IC47611 and/or related to a IC47611 associated disease or disorder, and based on one or more of the phenotypic information, the IC47611 information, and the acquired information, determining whether the subject has a IC47611 associated disease or disorder or a pre-disposition to a IC47611 associated disease or disorder. The method may further comprise the step of recommending a particular treatment for the disease, disorder or pre-disease condition.

The invention also includes an array comprising a IC47611 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be IC47611. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a IC47611 associated disease or disorder, progression of IC47611 associated disease or disorder, and processes, such a cellular transformation associated with the IC47611 associated disease or disorder.

The array is also useful for ascertaining the effect of the expression of a gene on the expression of other genes in the same cell or in different cells (e.g., ascertaining the effect of IC47611 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including IC47611) that could serve as a molecular target for diagnosis or therapeutic intervention.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human IC47611 cDNA

In this example, the identification and characterization of the gene encoding human IC47611 (clone Fbh47611FL) is described.

Isolation of the IC47611 cDNA

The invention is based, at least in part, on the discovery of a human gene encoding a novel protein, referred to herein as IC47611. The entire sequence of the human clone Fbh47611FL was determined and found to contain an open reading frame termed human “IC47611.” The nucleotide sequence of the human IC47611 gene is set forth in FIGS. 24A-C and in SEQ ID Nos:31 and 33. The amino acid sequence of the human IC47611 expression product is set forth in FIGS. 24A-C and in SEQ ID NO:32.

The nucleotide sequence encoding the human IC47611 protein is shown in FIGS. 24A-C and is set forth as SEQ ID NO:31. The protein encoded by this nucleic acid comprises about 453 amino acids and has the amino acid sequence shown in FIGS. 24A-C and set forth as SEQ ID NO:32. The coding region (open reading frame) of SEQ ID NO:31 is set forth as SEQ ID NO:33. Clone Fbh47611FL, comprising the coding region of human IC47611, was deposited with the American Type Culture Collection (ATCC®), 10801 University Boulevard, Manassas, Va. 20110-2209, on ______, and assigned Accession No. ______.

Analysis of the Human IC47611 Molecules

The amino acid sequence of human IC47611 was analyzed using the program PSORT (http://www.psort.nibb.ac.jp) to predict the localization of the proteins within the cell. This program assesses the presence of different targeting and localization amino acid sequences within the query sequence. The results of the analysis show that human IC47611 (SEQ ID NO:32) may be localized to the endoplasmic reticulum, to the mitochondrion, to the nucleus, or to secretory vesicles.

A search was performed against the Memsat database (FIG. 26), resulting in the identification of two transmembrane domains in the amino acid sequence of human IC47611 (SEQ ID NO:32) at about residues 108-132 and 180-204.

A search was also performed against the HMM database (FIG. 27) resulting in the identification of an inward-rectifier potassium channel (IRK) domain in the amino acid sequence of human IC47611 (SEQ ID NO:32) at about residues 72-399 (score=713.3).

A search was also performed against the ProDom database resulting in the identification of an inward-rectifier potassium channel (IRK) domain at about residues 74-403 (score=835), and an inward-rectifier potassium channel (IRK)-related domain at about residues 36-73 (score=185) of the amino acid sequence of human IC47611 (SEQ ID NO:32). The results of the search are set forth in FIG. 28.

Tissue Distribution of IC47611 mRNA

This example describes the tissue distribution of IC47611 mRNA, as is determined by Polymerase Chain Reaction (PCR) on cDNA libraries using oligonucleotide primers based on the human IC47611 sequence.

For in situ analysis, various tissues, e.g. tissues obtained from brain, are first frozen on dry ice. Ten-micrometer-thick sections of the tissues are postfixed with 4% formaldehyde in DEPC treated 1×phosphate-buffered saline at room temperature for 10 minutes before being rinsed twice in DEPC 1×phosphate-buffered saline and once in 0.1 M triethanolamine-HCl (pH 8.0). Following incubation in 0.25% acetic anhydride-0.1 M triethanolamine-HCl for 10 minutes, sections are rinsed in DEPC 2×SSC (1×SSC is 0.15M NaCl plus 0.015M sodium citrate). Tissue is then dehydrated through a series of ethanol washes, incubated in 100% chloroform for 5 minutes, and then rinsed in 100% ethanol for 1 minute and 95% ethanol for 1 minute and allowed to air dry.

Hybridizations are performed with ³⁵S-radiolabeled (5×10⁷ cpm/ml) cRNA probes. Probes are incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides are washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides are then rinsed with 2×SSC at room temperature, washed with 2×SSC at 50° C. for 1 hour, washed with 0.2×SSC at 55° C. for 1 hour, and 0.2×SSC at 60° C. for 1 hour. Sections are then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

### Example 2

### Expression of Recombinant IC47611 Protein in Bacterial Cells

In this example, IC47611 is expressed as a recombinant glutathione-S-transferase (GST) fusion polypeptide in E. coli and the fusion polypeptide is isolated and characterized. Specifically, IC47611 is fused to GST and this fusion polypeptide is expressed in E. coli, e.g., strain PEB199. Expression of the GST-IC47611 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant IC47611 Protein in COS Cells

To express the IC47611 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire IC47611 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the IC47611 DNA sequence is amplified by PCR using two primers. The 5′ primer contains the restriction site of interest followed by approximately twenty nucleotides of the IC47611 coding sequence starting from the initiation codon; the 3′ end sequence contains complementary sequences to the other restriction site of interest, a translation stop codon, the HA tag or FLAG tag and the last 20 nucleotides of the IC47611 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the IC47611 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the IC47611-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the IC47611 polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the IC47611 coding sequence is cloned directly into the polylinker of the pCDNA/Amp vector using the appropriate restriction sites. The resulting plasmid is transfected into COS cells in the manner described above, and the expression of the IC47611 polypeptide is detected by radiolabeling and immunoprecipitation using an IC47611 specific monoclonal antibody.

VI. 47615, A NOVEL HUMAN ION CHANNEL AND USES THEREOF

### Background of the Invention

The ion channel family of proteins is a large family of membrane-bound proteins responsible for a wide range of important transport and signaling functions in cells. The ion channel family includes at least three subfamilies: calcium ion channels (i.e., Ca channels), potassium channels (i.e., K channels) and sodium channels (Na channels). Members of the ion channel family are characterized by the presence of six (6) transmembrane helices in which the last two helices flank a loop which determines ion selectivity. In some subfamilies (e.g., Na channels) the domain is repeated four times, whereas in others (e.g., K channels) the protein forms as a tetramer in the membrane.

Calcium channel proteins are involved in the control of neurotransmitter release from neurons (Williams et al. (1992) Science 257:389-395), and play an important role in the regulation of a variety of cellular functions, including membrane excitability, muscle contraction and synaptic transmission (Mori et al. (1991) Nature 350:398-402). The calcium channel proteins are composed of four (4) tightly-coupled subunits (α1, α2, β and γ), the α1 subunit from each creating the pore for the import of extracellular calcium ions. The α1 subunit shares sequence characteristics with all voltage-dependent cation channels, and exploits the same 6-helix bundle structural motif. In both sodium and calcium channels, this motif is repeated 4 times within the sequence to give a 24-helix bundle. There are several tissue-specific pharmacologically and electrophysiologically distinct isoforms of calcium channels, coded for by separate genes in a multi-gene family. In skeletal muscle, each tightly-bound assembly of α, β and γ subunits associates with 4 others to form a pentameric macromolecule (Koch et al. (1990) J. Biol. Chem. 265:17786-17791). Examples of calcium channels include, but are not limited to, the low-voltage-gated channels and the high-voltage-gated channels. Calcium channels are described in, for example, Davila et al. (1999) Ann. N.Y. Acad. Sci. 868:102-17 and McEnery et al. (1998) J. Bioenergetics and Biomembranes 30(4):409-418, the contents of which are incorporated herein by reference.

Sodium channels are transmembrane (TM) voltage-dependent proteins responsible for the depolarizing phase of the action potential in most electrically excitable cells (George et al. (1992) Proc. Natl. Acad. Sci. USA 89:4893-4897). They may exist in 3 states (Noda et al. (1984) Nature 312:121-127): the resting state, where the channel is closed; the activated state, where the channel is open; and the inactivated state, where the channel is closed. Several different structurally and functionally distinct isoforms are found in mammals, coded for by a multigene family (Rogart et al. (1989) Proc. Natl. Acad. Sci. USA 86:8170-8174), these being responsible for the different types of sodium ion currents found in excitable tissues. The structure of sodium channels is based on 4 internal repeats of a 6-helix bundle (Noda et al. (1986) Nature 320:188-192) (in which 5 of the membrane-spanning segments are hydrophobic and the other is positively charged), forming a 24-helical bundle. The charged segments are believed to be localized within clusters formed by their 5 hydrophobic neighbors. It is postulated that the charged domain may be the voltage sensor region, possibly moving outward on depolarization, causing a conformational change. This model, proposed by (Noda et al. (1986) supra), contrasts with that of Sato and Matsumoto (1992) Biochem. Biophys. Res. Commun. 186:1158-1167), in which the TM segments are juxtaposed octagonally. The basic structural motif (the 6-helix bundle) is also found in potassium and calcium channels.

Potassium channels are the most diverse group of the ion channel family (possibly as a result of gene duplication and alternative splicing of the genes (Perney and Kaczmarek (1991) Curr. Opin. Cell. Biol. 3:663-670 and Luneau et al. (1991) FEBS Lett. 288:163-167). They are important in shaping the action potential, and in neuronal excitability and plasticity (Tempel et al. (1988) Nature 332:837-839). The potassium channel family is composed of several functionally distinct isoforms, which can be broadly separated into 2 groups (Stuehmer et al. (1989) EMBO J. 8:3225-3244). The first is the practically non-inactivating “delayed” group, the second the rapidly inactivating “transient” group. These are all highly similar proteins, with possibly only small amino acid changes causing the diversity of the voltage-dependent gating mechanism, channel conductance and toxin binding properties. Members of the potassium channel family vary in several ways. Some open in response to depolarization of the plasma membrane; others open in response to hyperpolarization or an increase in intracellular calcium concentration; some can be regulated by binding of a transmitter, together with intracellular kinases; and others are regulated by GTP-binding proteins or other second messengers (Schwarz et al. (1988) Nature 331:137-142 (1988). They are also involved in T-cell activation, and may have a role in target cell lysis by cytotoxic T-lymphocytes (Attali et al. (1992) J. Biol. Chem. 267:8650-8657 (1992). Potassium channels are transmembrane (TM) proteins that contain 6 membrane-spanning α-helical segments, 5 of which are hydrophobic, the other being positively charged. The charged segment is believed to be localized within a cluster formed by the hydrophobic helices. As with Na channels, it is postulated that the charged segment may constitute the voltage sensor region, possibly moving outward on depolarization, causing a conformational change. The 6-helix bundle is a common structural motif in sodium channels (in which it is repeated 4 times within the sequence to form a 24-helix bundle), and in calcium channels (where it also forms a 24-helix bundle, which itself is tightly bound to 3 different subunits).

Ion channels play a role in regulating ion transport and signaling in virtually every cell in the human body.

### Summary of the Invention

The present invention is based, at least in part, on the discovery of novel ion channel family members, referred to herein as ion channel 47615, or “IC47615” nucleic acid and protein molecules. The IC47615 molecules of the present invention are useful as targets for developing modulating agents to regulate a variety of cellular processes, including ion transport (e.g., ion conductance); membrane excitability and/or polarization; synaptic transmission; signal transduction; cell activation, proliferation, growth, differentiation and/or migration; and muscle contraction. Accordingly, in one aspect, this invention provides isolated nucleic acid molecules encoding IC47615 proteins or biologically active portions thereof, as well as nucleic acid fragments suitable as primers or hybridization probes for the detection of IC47615-encoding nucleic acids.

In one embodiment, an IC47615 nucleic acid molecule of the invention is at least 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the nucleotide sequence (e.g., to the entire length of the nucleotide sequence) shown in SEQ ID NO:34 or 36 or the nucleotide sequence of the DNA insert of the plasmid deposited with ATCC as Accession Number ______, or a complement thereof.

In a preferred embodiment, the isolated nucleic acid molecule includes the nucleotide sequence shown SEQ ID NO:34 or 36, or a complement thereof. In another embodiment, the nucleic acid molecule includes SEQ ID NO:36 and nucleotides 1-603 of SEQ ID NO:34. In yet another embodiment, the nucleic acid molecule includes SEQ ID NO:36 and nucleotides 1519-4003 of SEQ ID NO:34. In another preferred embodiment, the nucleic acid molecule consists of the nucleotide sequence shown in SEQ ID NO:34 or 36. In another preferred embodiment, the nucleic acid molecule includes a fragment of at least 50 nucleotides (e.g., 50 contiguous nucleotides) of the nucleotide sequence of SEQ ID NO:34 or 36, or a complement thereof.

In another embodiment, an IC47615 nucleic acid molecule includes a nucleotide sequence encoding a protein having an amino acid sequence sufficiently identical to the amino acid sequence of SEQ ID NO:35 or an amino acid sequence encoded by the DNA insert of the plasmid deposited with ATCC as Accession Number ______. In a preferred embodiment, an IC47615 nucleic acid molecule includes a nucleotide sequence encoding a protein having an amino acid sequence at least 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the entire length of the amino acid sequence of SEQ ID NO:35 or the amino acid sequence encoded by the DNA insert of the plasmid deposited with ATCC as Accession Number ______.

In another preferred embodiment, an isolated nucleic acid molecule encodes the amino acid sequence of human IC47615. In yet another preferred embodiment, the nucleic acid molecule includes a nucleotide sequence encoding a protein having the amino acid sequence of SEQ ID NO:35 or the amino acid sequence encoded by the DNA insert of the plasmid deposited with ATCC as Accession Number ______. In yet another preferred embodiment, the nucleic acid molecule is at least 50 nucleotides in length. In a further preferred embodiment, the nucleic acid molecule is at least 50 nucleotides in length and encodes a protein having an IC47615 activity (as described herein).

Another embodiment of the invention features nucleic acid molecules, preferably IC47615 nucleic acid molecules, which specifically detect IC47615 nucleic acid molecules relative to nucleic acid molecules encoding non-IC47615 proteins. For example, in one embodiment, such a nucleic acid molecule is at least 50-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 3000, 3500, 4000 or more nucleotides in length and hybridizes under stringent conditions to a nucleic acid molecule comprising the nucleotide sequence shown in SEQ ID NO:34, the nucleotide sequence of the DNA insert of the plasmid deposited with ATCC as Accession Number ______, or a complement thereof.

In preferred embodiments, the nucleic acid molecules are at least 15 (e.g., 15 contiguous) nucleotides in length and hybridize under stringent conditions to SEQ ID NO:34.

In other preferred embodiments, the nucleic acid molecule encodes a naturally occurring allelic variant of a polypeptide comprising the amino acid sequence of SEQ ID NO:35 or an amino acid sequence encoded by the DNA insert of the plasmid deposited with ATCC as Accession Number ______, wherein the nucleic acid molecule hybridizes to a complement of a nucleic acid molecule comprising SEQ ID NO:34 or 36 under stringent conditions.

Another embodiment of the invention provides an isolated nucleic acid molecule which is antisense to an IC47615 nucleic acid molecule, e.g., the coding strand of an IC47615 nucleic acid molecule.

Another aspect of the invention provides a vector comprising an IC47615 nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably an IC47615 protein, by culturing in a suitable medium, a host cell, e.g., a mammalian host cell such as a non-human mammalian cell, of the invention containing a recombinant expression vector, such that the protein is produced.

Another aspect of this invention features isolated or recombinant IC47615 proteins and polypeptides. In one embodiment, an isolated IC47615 protein has a transmembrane domain. In a preferred embodiment, an IC47615 protein includes at least one transmembrane domain and has an amino acid sequence at least about 50%, 55%, 60%, 65%, 67%, 68%, 70%, 75%, 80%, 85%, 90%, 95%, 96%, 97%, 98%, 99%, 99.2%, 99.3%, 99.4%, 99.5%, 99.6%, 99.7%, 99.8%, 99.9% or more identical to the amino acid sequence of SEQ ID NO:35, or the amino acid sequence encoded by the DNA insert of the plasmid deposited with ATCC as Accession Number ______. In another preferred embodiment, an IC47615 protein includes at least one transmembrane domain and has an IC47615 activity (as described herein).

In yet another preferred embodiment, an IC47615 protein includes at least one transmembrane domain and is encoded by a nucleic acid molecule having a nucleotide sequence which hybridizes under stringent hybridization conditions to a complement of a nucleic acid molecule comprising the nucleotide sequence of SEQ ID NO:34 or 36.

In another embodiment, the invention features fragments of the protein having the amino acid sequence of SEQ ID NO:35, wherein the fragment comprises at least 15 amino acids (e.g., contiguous amino acids) of the amino acid sequence of SEQ ID NO:35, or an amino acid sequence encoded by the DNA insert of the plasmid deposited with the ATCC as Accession Number ______. In another embodiment, an IC47615 protein has the amino acid sequence of SEQ ID NO:35.

In another embodiment, the invention features an IC47615 protein which is encoded by a nucleic acid molecule consisting of a nucleotide sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to a nucleotide sequence of SEQ ID NO:34 or 36, or a complement thereof. This invention further features an IC47615 protein, which is encoded by a nucleic acid molecule consisting of a nucleotide sequence which hybridizes under stringent hybridization conditions to a complement of a nucleic acid molecule comprising the nucleotide sequence,of SEQ ID NO:34 or 36, or a complement thereof.

The proteins of the present invention or portions thereof, e.g., biologically active portions thereof, can be operatively linked to a non-IC47615 polypeptide (e.g., heterologous amino acid sequences) to form fusion proteins. The invention further features antibodies, such as monoclonal or polyclonal antibodies, that specifically bind proteins of the invention, preferably IC47615 proteins. In addition, the IC47615 proteins or biologically active portions thereof can be incorporated into pharmaceutical compositions, which optionally include pharmaceutically acceptable carriers.

In another aspect, the present invention provides a method for detecting the presence of an IC47615 nucleic acid molecule, protein, or polypeptide in a biological sample by contacting the biological sample with an agent capable of detecting an IC47615 nucleic acid molecule, protein, or polypeptide such that the presence of an IC47615 nucleic acid molecule, protein or polypeptide is detected in the biological sample.

In another aspect, the present invention provides a method for detecting the presence of IC47615 activity in a biological sample by contacting the biological sample with an agent capable of detecting an indicator of IC47615 activity such that the presence of IC47615 activity is detected in the biological sample.

In another aspect, the invention provides a method for modulating IC47615 activity comprising contacting a cell capable of expressing IC47615 with an agent that modulates IC47615 activity such that IC47615 activity in the cell is modulated. In one embodiment, the agent inhibits IC47615 activity. In another embodiment, the agent stimulates IC47615 activity. In one embodiment, the agent is an antibody that specifically binds to an IC47615 protein. In another embodiment, the agent modulates expression of IC47615 by modulating transcription of an IC47615 gene or translation of an IC47615 mRNA. In yet another embodiment, the agent is a nucleic acid molecule having a nucleotide sequence that is antisense to the coding strand of an IC47615 mRNA or an IC47615 gene.

In one embodiment, the methods of the present invention are used to treat a subject having a disorder characterized by aberrant or unwanted IC47615 protein or nucleic acid expression or activity by administering an agent which is an IC47615 modulator to the subject. In one embodiment, the IC47615 modulator is an IC47615 protein. In another embodiment the IC47615 modulator is an IC47615 nucleic acid molecule. In yet another embodiment, the IC47615 modulator is a peptide, peptidomimetic, or other small molecule. In a preferred embodiment, the disorder characterized by aberrant or unwanted IC47615 protein or nucleic acid expression is a CNS disorder, such as a cognitive or neurodegenerative disorder. In another preferred embodiment, the disorder characterized by aberrant or unwanted IC47615 protein or nucleic acid expression is a cardiovascular disorder. In another preferred embodiment, the disorder characterized by aberrant or unwanted IC47615 protein or nucleic acid expression is a muscular disorder. In another embodiment, the disorder characterized by aberrant or unwanted IC47615 activity is a pain disorder. In another embodiment, the disorder characterized by aberrant or unwanted IC47615 activity is a cell proliferation, growth, differentiation, or migration disorder.

The present invention also provides diagnostic assays for identifying the presence or absence of a genetic alteration characterized by at least one of (i) aberrant modification or mutation of a gene encoding an IC47615 protein; (ii) mis-regulation of the gene; and (iii) aberrant post-translational modification of an IC47615 protein, wherein a wild-type form of the gene encodes a protein with an IC47615 activity.

In another aspect the invention provides methods for identifying a compound that binds to or modulates the activity of an IC47615 protein, by providing an indicator composition comprising an IC47615 protein having IC47615 activity, contacting the indicator composition with a test compound, and determining the effect of the test compound on IC47615 activity in the indicator composition to identify a compound that modulates the activity of an IC47615 protein.

Other features and advantages of the invention will be apparent from the following detailed description and claims.

### Detailed Description of the Invention

The present invention is based, at least in part, on the discovery of novel molecules, referred to herein as IC47615 (for ion channel 47615) nucleic acid and protein molecules, which are novel members of the ion channel family. These novel molecules are capable of, for example, modulating ion transport in an electrically excitable cell (e.g., a neuronal or muscle (e.g., cardiac muscle) cell), or in a non-electrically excitable cell, e.g., a spleen cell.

As used herein, the term “ion channel” includes a protein or polypeptide which is involved in receiving, conducting, and transmitting signals in an cell (e.g., an electrically excitable cell, for example, a neuronal or muscle cell). Ion channels can determine membrane excitability (the ability of, for example, a cell to respond to a stimulus and to convert it into a sensory impulse). Ion channels can also influence the resting potential of membranes, wave forms and frequencies of action potentials, and thresholds of excitation. Ion channels are typically expressed in electrically excitable cells, e.g., neuronal cells, and may form heteromultimeric structures (e.g., composed of more than one type of subunit). Ion channels may also be found in non-excitable cells (e.g., endothelial cells or spleen cells), where they may play a role in, for example, signal transduction. As the IC47615 molecules of the present invention may modulate ion channel mediated activities, they may be useful for developing novel diagnostic and therapeutic agents for ion channel associated disorders.

As used herein, an “ion channel associated disorder” includes a disorder, disease or condition which is characterized by a misregulation of an ion channel mediated activity. Ion channel associated disorders can detrimentally affect conveyance of sensory impulses from the periphery to the brain and/or conductance of motor impulses from the brain to the periphery; integration of reflexes; interpretation of sensory impulses; cellular proliferation, growth, differentiation, or migration, and emotional, intellectual (e.g., learning and memory), or motor processes. Examples of ion channel associated disorders include CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Ion channel disorders also include pain disorders. The IC47615 molecules of the present invention may be present on sensory neurons and, thus, may be involved in detecting, for example, noxious chemical, mechanical, or thermal stimuli and transducing this information into membrane depolarization events. Thus, the IC47615 molecules by participating in pain signaling mechanisms, may modulate pain elicitation and act as targets for developing novel diagnostic targets and therapeutic agents to control pain.

Further examples of ion channel associated disorders include cardiovascular system disorders. Cardiovascular system disorders in which the IC47615 molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia. IC47615-mediated or related disorders also include disorders of the musculoskeletal system such as paralysis and muscle weakness, e.g., ataxia, myotonia, and myokymia.

Ion channel disorders also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The IC47615 molecules of the present invention are involved in signal transduction mechanisms, which are known to be involved in cellular growth, differentiation, and migration processes. Thus, the IC47615 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementia; hepatic disorders; cardiovascular disorders; and hematopoietic and/or myeloproliferative disorders.

IC47615-associated or related disorders also include disorders of tissues in which IC47615 protein is expressed.

As used herein, an “ion channel mediated activity” includes an activity which involves an ion channel, e.g., an ion channel associated with receiving, conducting, and transmitting signals, in electrically excitable or non-electrically excitable cells. Ion channel mediated activities include release of neurotransmitters or second messenger molecules, e.g., dopamine or norepinephrine, from cells, e.g., neuronal cells; modulation of resting potential of membranes, wave forms and frequencies of action potentials, and thresholds of excitation; participation in signal transduction pathways; and modulation of processes such as integration of sub-threshold synaptic responses and the conductance of back-propagating action potentials in, for example, neuronal cells (e.g., changes in those action potentials resulting in a morphological or differentiative response in the cell).

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., monkey proteins. Members of a family may also have common functional characteristics.

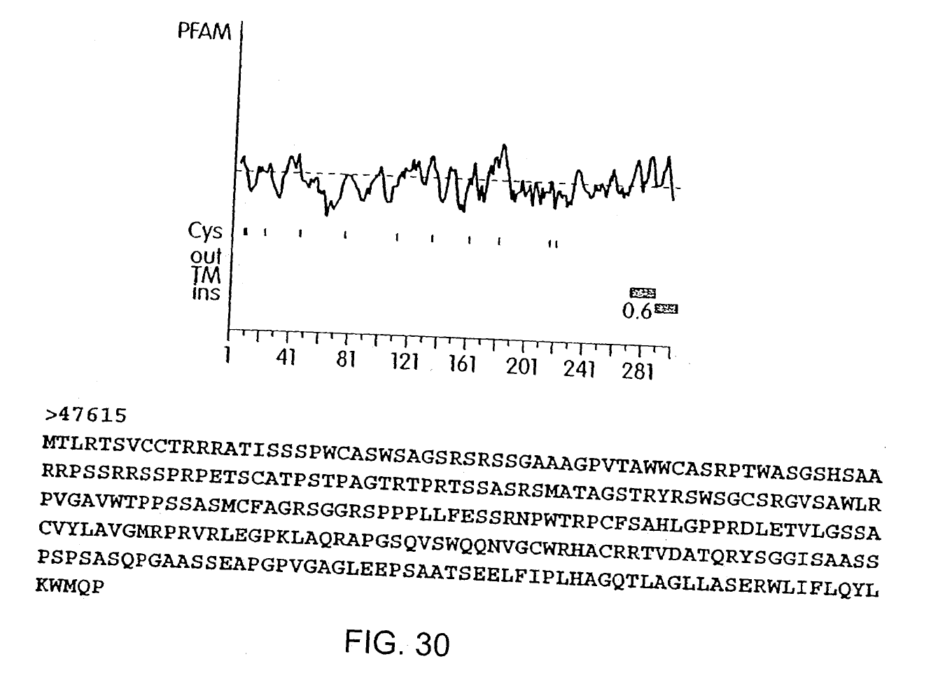

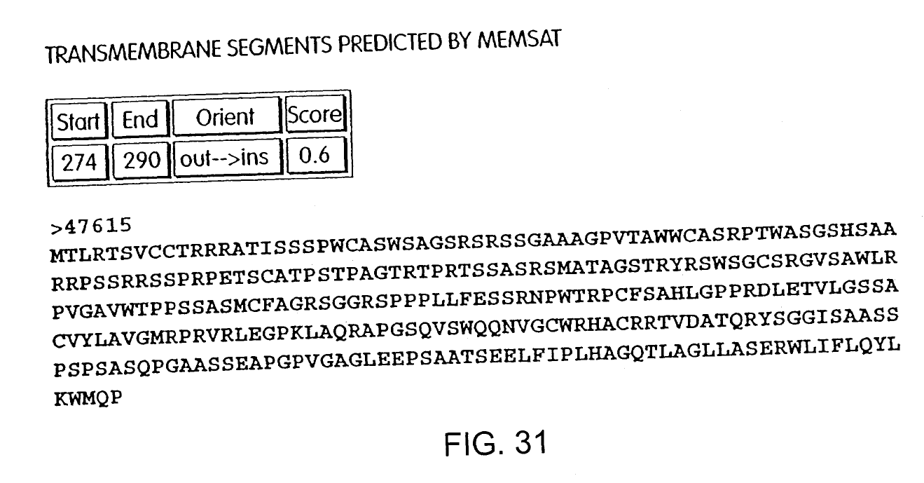

For example, the family of IC47615 proteins comprises at least one “transmembrane domain”. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al. (1996) Annu. Rev. Neurosci. 19:235-263, the contents of which are incorporated herein by reference. Amino acid residues 274-290 of the native IC47615 protein are predicted to comprise a transmembrane domain (see FIGS. 30 and 31). Accordingly, IC47615 proteins having at least 50-60% homology, preferably about 60-70%, more preferably about 70-80%, or about 80-90% homology with a transmembrane domain of human IC47615 are within the scope of the invention.

Isolated proteins of the present invention, preferably IC47615 proteins, have an amino acid sequence sufficiently identical to the amino acid sequence of SEQ ID NO:35 or are encoded by a nucleotide sequence sufficiently identical to SEQ ID NO:34 or 36. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, an “IC47615 activity”, “biological activity of IC47615” or “functional activity of IC47615”, refers to an activity exerted by an IC47615 protein, polypeptide or nucleic acid molecule on an IC47615 responsive cell or tissue, or on an IC47615 protein substrate, as determined in vivo, or in vitro, according to standard techniques. In one embodiment, an IC47615 activity is a direct activity, such as an association with an IC47615-target molecule. As used herein, a “target molecule” or “binding partner” is a molecule with which an IC47615 protein binds or interacts in nature, such that IC47615-mediated function is achieved. An IC47615 target molecule can be a non-IC47615 molecule or an IC47615 protein or polypeptide of the present invention. In an exemplary embodiment, an IC47615 target molecule is an IC47615 ligand, e.g., an ion channel pore-forming subunit or an ion channel ligand. Alternatively, an IC47615 activity is an indirect activity, such as a cellular signaling activity mediated by interaction of the IC47615 protein with an IC47615 ligand. The biological activities of IC47615 are described herein. For example, the IC47615 proteins of the present invention can have one or more of the following activities: (1) modulation of membrane excitability, (2) modulation of intracellular ion concentration, (3) modulation of membrane polarization (e.g., membrane polarization and/or depolarization), (4) modulation of action potential, (5) modulation of cellular signal transduction, (6) modulation of neurotransmitter release (e.g., from neuronal cells), (7) modulation of synaptic transmission, (8) modulation of neuronal excitability and/or plasticity, (9) modulation of muscle contraction, (10) modulation of cell activation (e.g., T cell activation), and/or (11) modulation of cellular proliferation, growth, migration and/or differentiation.

Accordingly, another embodiment of the invention features isolated IC47615 proteins and polypeptides having an IC47615 activity. Preferred proteins are IC47615 proteins having one or more transmembrane domains, and, preferably, an IC47615 activity.

Additional preferred proteins have one or more transmembrane domains, and are, preferably, encoded by a nucleic acid molecule having a nucleotide sequence which hybridizes under stringent hybridization conditions to a complement of a nucleic acid molecule comprising the nucleotide sequence of SEQ ID NO:34 or 36.

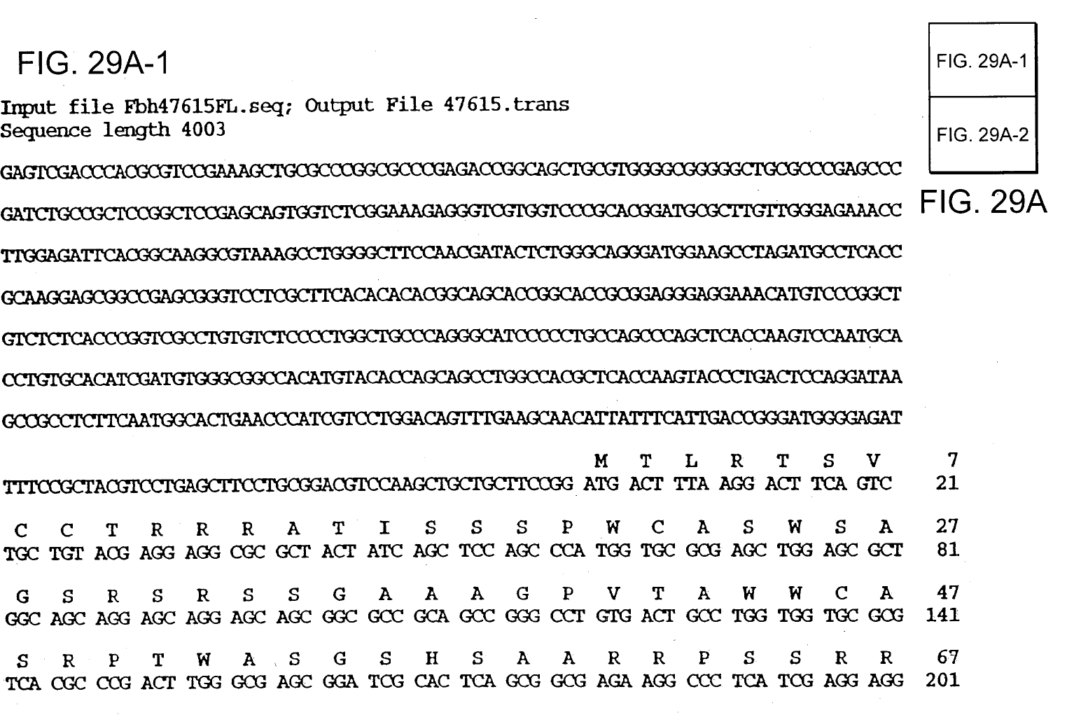

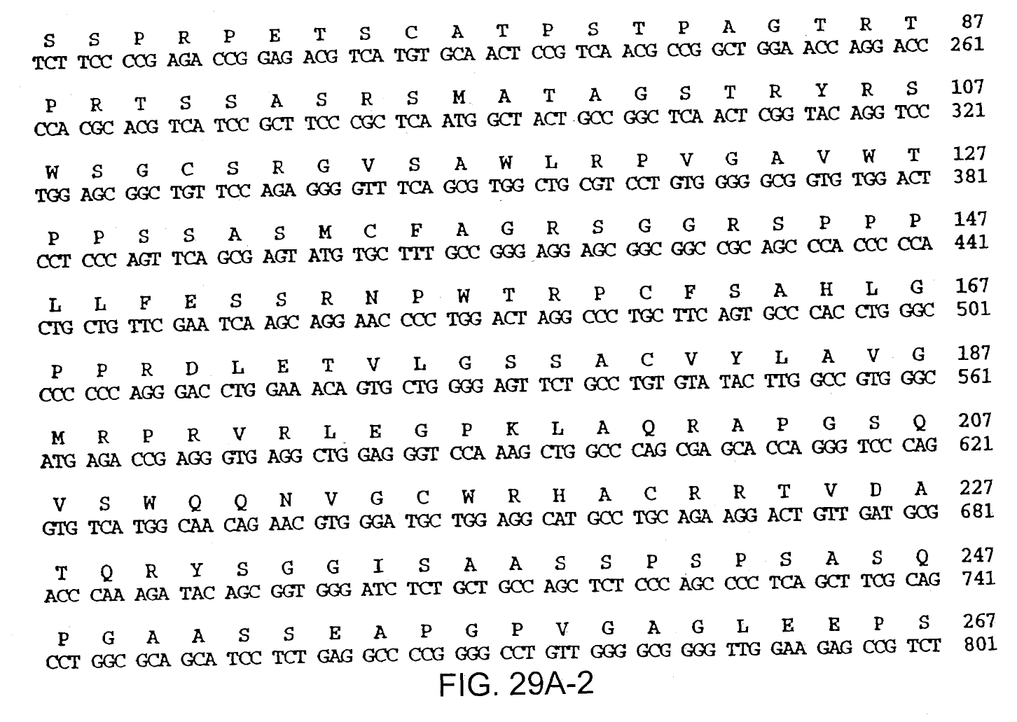

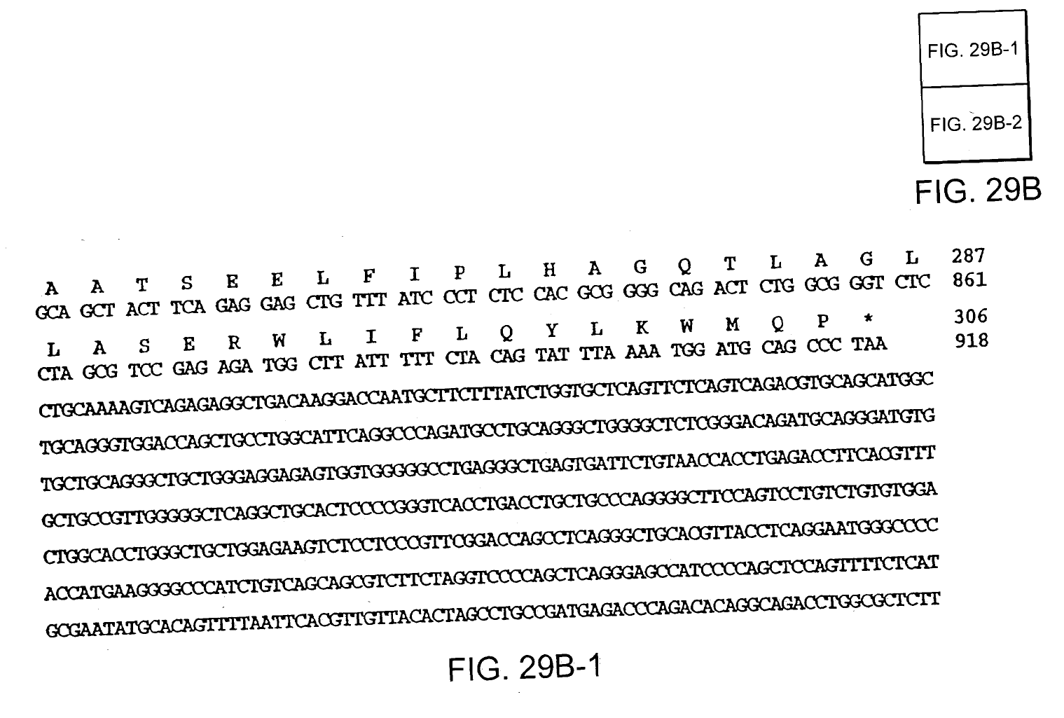

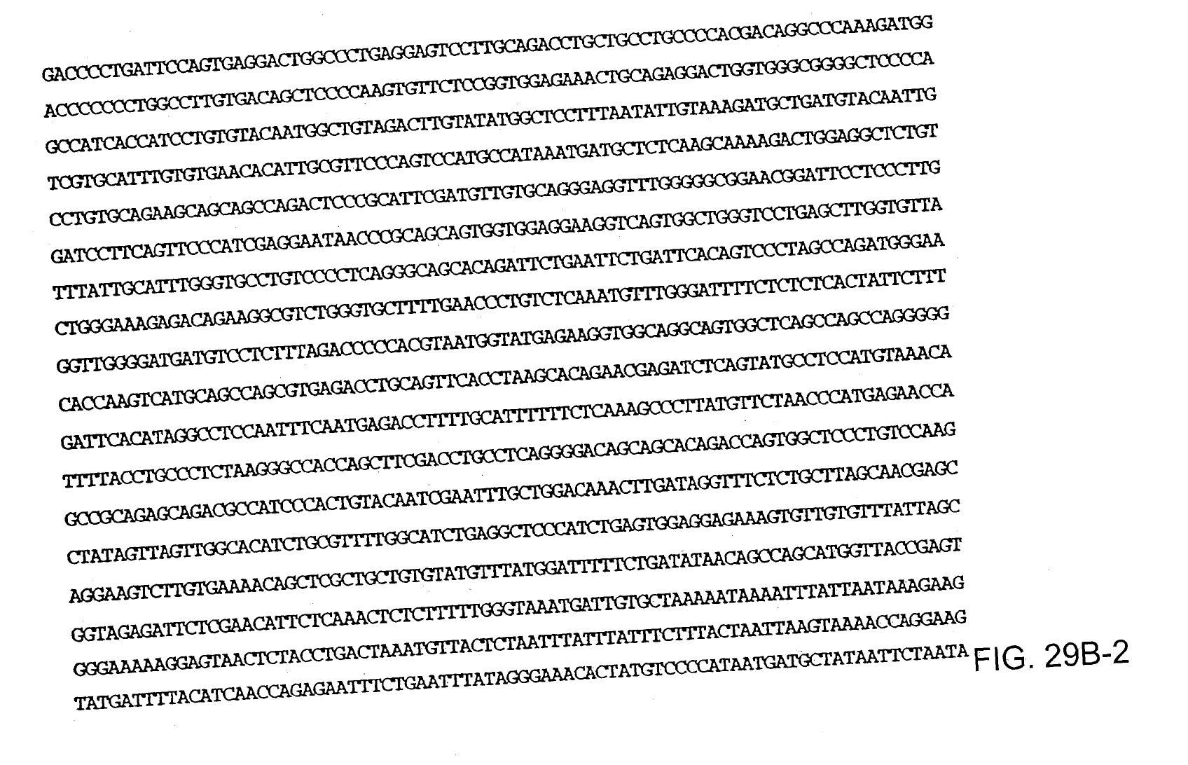

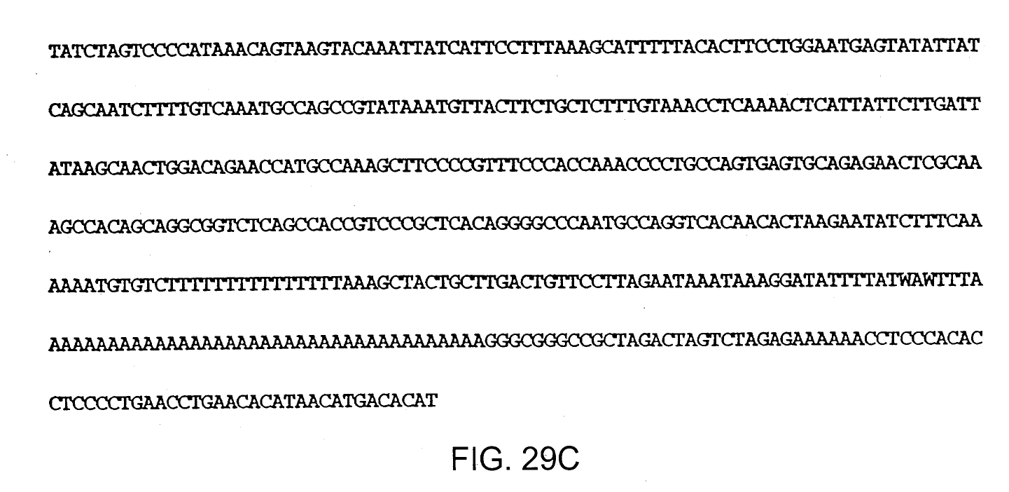

The nucleotide sequence of the isolated human IC47615 cDNA and the predicted amino acid sequence of the human IC47615 polypeptide are shown in FIGS. 29A-C and in SEQ ID NOs:34 and 35, respectively. A plasmid containing the nucleotide sequence encoding human IC47615 was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The human IC47615 gene, which is approximately 4003 nucleotides in length, encodes a protein having a molecular weight of approximately 34.7 kD and which is approximately 305 amino acid residues in length.

Various aspects of the invention are described in further detail in the following subsections:

I. Isolated Nucleic Acid Molecules

One aspect of the invention pertains to isolated nucleic acid molecules that encode IC47615 proteins or biologically active portions thereof, as well as nucleic acid fragments sufficient for use as hybridization probes to identify IC47615-encoding nucleic acid molecules (e.g., IC47615 mRNA) and fragments for use as PCR primers for the amplification or mutation of IC47615 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated IC47615 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:34 or 36, or the nucleotide sequence of the DNA insert of the plasmid deposited with ATCC as Accession Number ______, or a portion thereof, can be isolated using standard molecular biology techniques and the sequence information provided herein. Using all or portion of the nucleic acid sequence of SEQ ID NO:34 or 36, or the nucleotide sequence of the DNA insert of the plasmid deposited with ATCC as Accession Number ______, as a hybridization probe, IC47615 nucleic acid molecules can be isolated using standard hybridization and cloning techniques (e.g., as described in Sambrook, J. et al., Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:34 or 36, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:34 or 36, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to IC47615 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:34. This cDNA comprises sequences encoding the human IC47615 protein (i.e., “the coding region”, from nucleotides 604-1518), as well as 5′ untranslated sequences (nucleotides 1-603) and 3′ untranslated sequences (nucleotides 1519-4003). Alternatively, the nucleic acid molecule can comprise only the coding region of SEQ ID NO:34 (e.g., nucleotides 604-1518, corresponding to SEQ ID NO:36). In another embodiment, the nucleic acid molecule can comprise the coding region of SEQ ID NO:34 (e.g., nucleotides 604-1518, corresponding to SEQ ID NO:36), as well as a stop codon (e.g., nucleotides 1519-1522 of SEQ ID NO:34).

In another preferred embodiment, an isolated nucleic acid molecule of the invention comprises a nucleic acid molecule which is a complement of the nucleotide sequence shown in SEQ ID NO:34 or 36, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:34 or 36, or the nucleotide sequence of the DNA insert of the plasmid deposited with ATCC as Accession Number ______, is one which is sufficiently complementary to the nucleotide sequence shown in SEQ ID NO:34 or 36, or the nucleotide sequence of the DNA insert of the plasmid deposited with ATCC as Accession Number _______, such that it can hybridize to the nucleotide sequence shown in SEQ ID NO:34 or 36, or the nucleotide sequence of the DNA insert of the plasmid deposited with ATCC as Accession Number ______, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the entire length of the nucleotide sequence shown in SEQ ID NO:34 or 36, or the entire length of the nucleotide sequence of the DNA insert of the plasmid deposited with ATCC as Accession Number ______, or a portion of any of these nucleotide sequences.

Moreover, the nucleic acid molecule of the invention can comprise only a portion of the nucleic acid sequence of SEQ ID NO:34 or 36, or the nucleotide sequence of the DNA insert of the plasmid deposited with ATCC as Accession Number ______, for example, a fragment which can be used as a probe or primer or a fragment encoding a portion of an IC47615 protein, e.g., a biologically active portion of an IC47615 protein. The nucleotide sequence determined from the cloning of the IC47615 gene allows for the generation of probes and primers designed for use in identifying and/or cloning other IC47615 family members, as well as IC47615 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:34 or 36, or the nucleotide sequence of the DNA insert of the plasmid deposited with ATCC as Accession Number ______, of an anti-sense sequence of SEQ ID NO:34 or 36, or the nucleotide sequence of the DNA insert of the plasmid deposited with ATCC as Accession Number ______, or of a naturally occurring allelic variant or mutant of SEQ ID NO:34 or 36, or the nucleotide sequence of the DNA insert of the plasmid deposited with ATCC as Accession Number ______. In one embodiment, a nucleic acid molecule of the present invention comprises a nucleotide sequence which is greater than 50-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 3000, 3500, 4000 or more nucleotides in length and hybridizes under stringent hybridization conditions to a nucleic acid molecule of SEQ ID NO:34 or 36, or the nucleotide sequence of the DNA insert of the plasmid deposited with ATCC as Accession Number ______.

Probes based on the IC47615 nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an IC47615 protein, such as by measuring a level of an IC47615-encoding nucleic acid in a sample of cells from a subject e.g., detecting IC47615 mRNA levels or determining whether a genomic IC47615 gene has been mutated or deleted.

A nucleic acid fragment encoding a “biologically active portion of an IC47615 protein” can be prepared by isolating a portion of the nucleotide sequence of SEQ ID NO:34 or 36, or the nucleotide sequence of the DNA insert of the plasmid deposited with ATCC as Accession Number ______, which encodes a polypeptide having an IC47615 biological activity (the biological activities of the IC47615 proteins are described herein), expressing the encoded portion of the IC47615 protein (e.g., by recombinant expression in vitro) and assessing the activity of the encoded portion of the IC47615 protein.

The invention further encompasses nucleic acid molecules that differ from the nucleotide sequence shown in SEQ ID NO:34 or 36, or the nucleotide sequence of the DNA insert of the plasmid deposited with ATCC as Accession Number ______, due to degeneracy of the genetic code and thus encode the same IC47615 proteins as those encoded by the nucleotide sequence shown in SEQ ID NO:34 or 36, or the nucleotide sequence of the DNA insert of the plasmid deposited with ATCC as Accession Number ______. In another embodiment, an isolated nucleic acid molecule of the invention has a nucleotide sequence encoding a protein having an amino acid sequence shown in SEQ ID NO:35.

In addition to the IC47615 nucleotide sequences shown in SEQ ID NO:34 or 36, or the nucleotide sequence of the DNA insert of the plasmid deposited with ATCC as Accession Number ______, it will be appreciated by those skilled in the art that DNA sequence polymorphisms that lead to changes in the amino acid sequences of the IC47615 proteins may exist within a population (e.g., the human population). Such genetic polymorphism in the IC47615 genes may exist among individuals within a population due to natural allelic variation. As used herein, the terms “gene” and “recombinant gene” refer to nucleic acid molecules which include an open reading frame encoding an IC47615 protein, preferably a mammalian IC47615 protein, and can further include non-coding regulatory sequences, and introns.

Allelic variants of human IC47615 include both functional and non-functional IC47615 proteins. Functional allelic variants are naturally occurring amino acid sequence variants of the human IC47615 protein that maintain the ability to bind an IC47615 ligand or substrate and/or modulate cell proliferation and/or migration mechanisms. Functional allelic variants will typically contain only conservative substitution of one or more amino acids of SEQ ID NO:35, or substitution, deletion or insertion of non-critical residues in non-critical regions of the protein.

Non-functional allelic variants are naturally occurring amino acid sequence variants of the human IC47615 protein that do not have the ability to either bind an IC47615 ligand and/or modulate any of the IC47615 activities described herein. Non-functional allelic variants will typically contain a non-conservative substitution, a deletion, or insertion or premature truncation of the amino acid sequence of SEQ ID NO:35, or a substitution, insertion or deletion in critical residues or critical regions.

The present invention further provides non-human orthologues of the human IC47615 protein. Orthologues of the human IC47615 protein are proteins that are isolated from non-human organisms and possess the same IC47615 ligand binding and/or modulation of membrane excitability activities of the human IC47615 protein. Orthologues of the human IC47615 protein can readily be identified as comprising an amino acid sequence that is substantially identical to SEQ ID NO:35.

Moreover, nucleic acid molecules encoding other IC47615 family members and, thus, which have a nucleotide sequence which differs from the IC47615 sequences of SEQ ID NO:34 or 36, or the nucleotide sequence of the DNA insert of the plasmid deposited with ATCC as Accession Number ______ are intended to be within the scope of the invention. For example, another IC47615 cDNA can be identified based on the nucleotide sequence of human IC47615. Moreover, nucleic acid molecules encoding IC47615 proteins from different species, and which, thus, have a nucleotide sequence which differs from the IC47615 sequences of SEQ ID NO:34 or 36, or the nucleotide sequence of the DNA insert of the plasmid deposited with ATCC as Accession Number ______ are intended to be within the scope of the invention. For example, a mouse IC47615 cDNA can be identified based on the nucleotide sequence of a human IC47615.

Nucleic acid molecules corresponding to natural allelic variants and homologues of the IC47615 cDNAs of the invention can be isolated based on their homology to the IC47615 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the IC47615 cDNAs of the invention can further be isolated by mapping to the same chromosome or locus as the IC47615 gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:34 or 36, or the nucleotide sequence of the DNA insert of the plasmid deposited with ATCC as Accession Number ______. In other embodiment, the nucleic acid is at least 50-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 3000, 3500, 4000 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4×sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, T_{m(° C.)=}81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

Preferably, an isolated nucleic acid molecule of the invention that hybridizes under stringent conditions to the sequence of SEQ ID NO:34 or 36, and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the IC47615 sequences that may exist in the population, the skilled artisan will further appreciate that changes can be introduced by mutation into the nucleotide sequences of SEQ ID NO:34 or 36, or the nucleotide sequence of the DNA insert of the plasmid deposited with ATCC as Accession Number ______, thereby leading to changes in the amino acid sequence of the encoded IC47615 proteins, without altering the functional ability of the IC47615 proteins. For example, nucleotide substitutions leading to amino acid substitutions at “non-essential” amino acid residues can be made in the sequence of SEQ ID NO:34 or 36, or the nucleotide sequence of the DNA insert of the plasmid deposited with ATCC as Accession Number ______. A “non-essential” amino acid residue is a residue that can be altered from the wild-type sequence of IC47615 (e.g., the sequence of SEQ ID NO:35) without altering the biological activity, whereas an “essential” amino acid residue is required for biological activity. For example, amino acid residues that are conserved among the IC47615 proteins of the present invention, e.g., those present in a transmembrane domain, are predicted to be particularly unamenable to alteration. Furthermore, additional amino acid residues that are conserved between the IC47615 proteins of the present invention and other members of the IC47615 family are not likely to be amenable to alteration.

Accordingly, another aspect of the invention pertains to nucleic acid molecules encoding IC47615 proteins that contain changes in amino acid residues that are not essential for activity. Such IC47615 proteins differ in amino acid sequence from SEQ ID NO:35, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to SEQ ID NO:35.

An isolated nucleic acid molecule encoding an IC47615 protein identical to the protein of SEQ ID NO:35, can be created by introducing one or more nucleotide substitutions, additions or deletions into the nucleotide sequence of SEQ ID NO:34 or 36, or the nucleotide sequence of the DNA insert of the plasmid deposited with ATCC as Accession Number ______, such that one or more amino acid substitutions, additions or deletions are introduced into the encoded protein. Mutations can be introduced into SEQ ID NO:34 or 36, or the nucleotide sequence of the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an IC47615 protein is preferably replaced with another amino acid residue from the same side chain family. Alternatively, in another embodiment, mutations can be introduced randomly along all or part of an IC47615 coding sequence, such as by saturation mutagenesis, and the resultant mutants can be screened for IC47615 biological activity to identify mutants that retain activity. Following mutagenesis of SEQ ID NO:34 or 36, or the nucleotide sequence of the DNA insert of the plasmid deposited with ATCC as Accession Number ______,the encoded protein can be expressed recombinantly and the activity of the protein can be determined.

In a preferred embodiment, a mutant IC47615 protein can be assayed for the ability to (1) modulate membrane excitability, (2) regulate intracellular ion concentration, (3) modulate membrane polarization (e.g., membrane polarization and/or depolarization), (4) regulate action potential, (5) regulate cellular signal transduction, (6) regulate neurotransmitter release (e.g., from neuronal cells), (7) modulate synaptic transmission, (8) regulate neuronal excitability and/or plasticity, (9) regulate muscle contraction, (10) regulate cell activation and (11) regulate cellular proliferation, growth, migration and/or differentiation.

In addition to the nucleic acid molecules encoding IC47615 proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire IC47615 coding strand, or to only a portion thereof. In one embodiment, an antisense nucleic acid molecule is antisense to a “coding region” of the coding strand of a nucleotide sequence encoding IC47615. The term “coding region” refers to the region of the nucleotide sequence comprising codons which are translated into amino acid residues (e.g., the coding region of human IC47615 corresponds to SEQ ID NO:36). In another embodiment, the antisense nucleic acid molecule is antisense to a “noncoding region” of the coding strand of a nucleotide sequence encoding IC47615. The term “noncoding region” refers to 5′ and 3′ sequences which flank the coding region that are not translated into amino acids (i.e., also referred to as 5′ and 3′ untranslated regions).

Given the coding strand sequences encoding IC47615 disclosed herein (e.g., SEQ ID NO:36), antisense nucleic acids of the invention can be designed according to the rules of Watson and Crick base pairing. The antisense nucleic acid molecule can be complementary to the entire coding region of IC47615 mRNA, but more preferably is an oligonucleotide which is antisense to only a portion of the coding or noncoding region of IC47615 mRNA. For example, the antisense oligonucleotide can be complementary to the region surrounding the translation start site of IC47615 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an IC47615 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave IC47615 mRNA transcripts to thereby inhibit translation of IC47615 mRNA. A ribozyme having specificity for an IC47615-encoding nucleic acid can be designed based upon the nucleotide sequence of an IC47615 cDNA disclosed herein (i.e., SEQ ID NO:34 or 36, or the nucleotide sequence of the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in an IC47615-encoding mRNA. See, e.g., Cech et al. U.S. Pat. No. 4,987,071; and Cech et al. U.S. Pat. No. 5,116,742. Alternatively, IC47615 mRNA can be used to select a catalytic RNA having a specific ribonuclease activity from a pool of RNA molecules. See, e.g., Bartel, D. and Szostak, J. W. (1993) Science 261:1411-1418.

Alternatively, IC47615 gene expression can be inhibited by targeting nucleotide sequences complementary to the regulatory region of the IC47615 (e.g., the IC47615 promoter and/or enhancers; e.g., nucleotides 1-603 of SEQ ID NO:34) to form triple helical structures that prevent transcription of the IC47615 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioessays 14(12):807-15.

In yet another embodiment, the IC47615 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci USA 93:14670-675.

PNAs of IC47615 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of IC47615 nucleic acid molecules can also be used in the analysis of single base pair mutations in a gene, (e.g., by PNA-directed PCR clamping); as ‘artificial restriction enzymes’ when used in combination with other enzymes (e.g, S1 nucleases (Hyrup and Nielsen (1996) supra)); or as probes or primers for DNA sequencing or hybridization (Hyrup and Nielsen (1996) supra; Perry-O'Keefe et al. (1996) supra).

In another embodiment, PNAs of IC47615 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of IC47615 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup and Nielsen (1996) supra). The synthesis of PNA-DNA chimeras can be performed as described in Hyrup and Nielsen (1996) supra and Finn, P. J. et al. (1996) Nucleic Acids Res. 24(17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acids Res. 17:5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous IC47615 gene within a cell line or microorganism may be modified by inserting a heterologous DNA regulatory element into the genome of a stable cell line or cloned microorganism such that the inserted regulatory element is operatively linked with the endogenous IC47615 gene. For example, an endogenous IC47615 gene which is normally “transcriptionally silent”, i.e., an IC47615 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous IC47615 gene may be activated by insertion of a promiscuous regulatory element that works across cell types.

A heterologous regulatory element may be inserted into a stable cell line or cloned microorganism, such that it is operatively linked with an endogenous IC47615 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated IC47615 Proteins and Anti-IC47615 Antibodies

One aspect of the invention pertains to isolated IC47615 proteins, and biologically active portions thereof, as well as polypeptide fragments suitable for use as immunogens to raise anti-IC47615 antibodies. In one embodiment, native IC47615 proteins can be isolated from cells or tissue sources by an appropriate purification scheme using standard protein purification techniques. In another embodiment, IC47615 proteins are produced by recombinant DNA techniques. Alternative to recombinant expression, an IC47615 protein or polypeptide can be synthesized chemically using standard peptide synthesis techniques.

An “isolated” or “purified” protein or biologically active portion thereof is substantially free of cellular material or other contaminating proteins from the cell or tissue source from which the IC47615 protein is derived, or substantially free from chemical precursors or other chemicals when chemically synthesized. The language “substantially free of cellular material” includes preparations of IC47615 protein in which the protein is separated from cellular components of the cells from which it is isolated or recombinantly produced. In one embodiment, the language “substantially free of cellular material” includes preparations of IC47615 protein having less than about 30% (by dry weight) of non-IC47615 protein (also referred to herein as a “contaminating protein”), more preferably less than about 20% of non-IC47615 protein, still more preferably less than about 10% of non-IC47615 protein, and most preferably less than about 5% non-IC47615 protein. When the IC47615 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of IC47615 protein in which the protein is separated from chemical precursors or other chemicals which are involved in the synthesis of the protein. In one embodiment, the language “substantially free of chemical precursors or other chemicals” includes preparations of IC47615 protein having less than about 30% (by dry weight) of chemical precursors or non-IC47615 chemicals, more preferably less than about 20% chemical precursors or non-IC47615 chemicals, still more preferably less than about 10% chemical precursors or non-IC47615 chemicals, and most preferably less than about 5% chemical precursors or non-IC47615 chemicals.

As used herein, a “biologically active portion” of an IC47615 protein includes a fragment of an IC47615 protein which participates in an interaction between an IC47615 molecule and a non-IC47615 molecule. Biologically active portions of an IC47615 protein include peptides comprising amino acid sequences sufficiently identical to or derived from the amino acid sequence of the IC47615 protein, e.g., the amino acid sequence shown in SEQ ID NO:35, which include less amino acids than the full length IC47615 proteins, and exhibit at least one activity of an IC47615 protein. Typically, biologically active portions comprise a domain or motif with at least one activity of the IC47615 protein, e.g., modulating membrane excitability. A biologically active portion of an IC47615 protein can be a polypeptide which is, for example, 25, 50, 75, 100, 125, 150, 175, 200 or more amino acids in length. Biologically active portions of an IC47615 protein can be used as targets for developing agents which modulate an IC47615 mediated activity, e.g., modulation of membrane excitability.

In one embodiment, a biologically active portion of an IC47615 protein comprises at least one transmembrane domain. Moreover, other biologically active portions, in which other regions of the protein are deleted, can be prepared by recombinant techniques and evaluated for one or more of the functional activities of a native IC47615 protein.

In a preferred embodiment, the IC47615 protein has an amino acid sequence shown in SEQ ID NO:35. In other embodiments, the IC47615 protein is substantially identical to SEQ ID NO:35, and retains the functional activity of the protein of SEQ ID NO:35, yet differs in amino acid sequence due to natural allelic variation or mutagenesis, as described in detail in subsection I above. Accordingly, in another embodiment, the IC47615 protein is a protein which comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to SEQ ID NO:35.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the IC47615 amino acid sequence of SEQ ID NO:35 having 305 amino acid residues, at least 92, preferably at least 122, more preferably at least 153, even more preferably at least 183, and even more preferably at least 214, 244, 275 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to IC47615 nucleic acid molecules of the invention. BLAST protein searches can be performed with the XBLAST program, score=100, wordlength=3 to obtain amino acid sequences homologous to IC47615 protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides IC47615 chimeric or fusion proteins. As used herein, an IC47615 “chimeric protein” or “fusion protein” comprises an IC47615 polypeptide operatively linked to a non-IC47615 polypeptide. An “IC47615 polypeptide” refers to a polypeptide having an amino acid sequence corresponding to IC47615, whereas a “non-IC47615 polypeptide” refers to a polypeptide having an amino acid sequence corresponding to a protein which is not substantially homologous to the IC47615 protein, e.g., a protein which is different from the IC47615 protein and which is derived from the same or a different organism. Within an IC47615 fusion protein the IC47615 polypeptide can correspond to all or a portion of an IC47615 protein. In a preferred embodiment, an IC47615 fusion protein comprises at least one biologically active portion of an IC47615 protein. In another preferred embodiment, an IC47615 fusion protein comprises at least two biologically active portions of an IC47615 protein. Within the fusion protein, the term “operatively linked” is intended to indicate that the IC47615 polypeptide and the non-IC47615 polypeptide are fused in-frame to each other. The non-IC47615 polypeptide can be fused to the N-terminus or C-terminus of the IC47615 polypeptide.

For example, in one embodiment, the fusion protein is a GST-IC47615 fusion protein in which the IC47615 sequences are fused to the C-terminus of the GST sequences. Such fusion proteins can facilitate the purification of recombinant IC47615.

In another embodiment, the fusion protein is an IC47615 protein containing a heterologous signal sequence at its N-terminus. In certain host cells (e.g., mammalian host cells), expression and/or secretion of IC47615 can be increased through use of a heterologous signal sequence.

The IC47615 fusion proteins of the invention can be incorporated into pharmaceutical compositions and administered to a subject in vivo. The IC47615 fusion proteins can be used to affect the bioavailability of an IC47615 substrate. Use of IC47615 fusion proteins may be useful therapeutically for the treatment of disorders caused by, for example, (i) aberrant modification or mutation of a gene encoding an IC47615 protein; (ii) mis-regulation of the IC47615 gene; and (iii) aberrant post-translational modification of an IC47615 protein.

Moreover, the IC47615-fusion proteins of the invention can be used as immunogens to produce anti-IC47615 antibodies in a subject, to purify IC47615 ligands and in screening assays to identify molecules which inhibit the interaction of IC47615 with an IC47615 substrate.

Preferably, an IC47615 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An IC47615-encoding nucleic acid can be cloned into such an expression vector such that the fusion moiety is linked in-frame to the IC47615 protein.

The present invention also pertains to variants of the IC47615 proteins which function as either IC47615 agonists (mimetics) or as IC47615 antagonists. Variants of the IC47615 proteins can be generated by mutagenesis, e.g., discrete point mutation or truncation of an IC47615 protein. An agonist of the IC47615 proteins can retain substantially the same, or a subset, of the biological activities of the naturally occurring form of an IC47615 protein. An antagonist of an IC47615 protein can inhibit one or more of the activities of the naturally occurring form of the IC47615 protein by, for example, competitively modulating an IC47615-mediated activity of an IC47615 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the IC47615 protein.

In one embodiment, variants of an IC47615 protein which function as either IC47615 agonists (mimetics) or as IC47615 antagonists can be identified by screening combinatorial libraries of mutants, e.g., truncation mutants, of an IC47615 protein for IC47615 protein agonist or antagonist activity. In one embodiment, a variegated library of IC47615 variants is generated by combinatorial mutagenesis at the nucleic acid level and is encoded by a variegated gene library. A variegated library of IC47615 variants can be produced by, for example, enzymatically ligating a mixture of synthetic oligonucleotides into gene sequences such that a degenerate set of potential IC47615 sequences is expressible as individual polypeptides, or alternatively, as a set of larger fusion proteins (e.g., for phage display) containing the set of IC47615 sequences therein. There are a variety of methods that can be used to produce libraries of potential IC47615 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential IC47615 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acids Res. 11:477.

In addition, libraries of fragments of an IC47615 protein coding sequence can be used to generate a variegated population of IC47615 fragments for screening and subsequent selection of variants of an IC47615 protein. In one embodiment, a library of coding sequence fragments can be generated by treating a double stranded PCR fragment of an IC47615 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the IC47615 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of IC47615 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify IC47615 variants (Arkin and Youvan (1992) Proc. Natl. Acad. Sci. USA 89:7811-7815; Delagrave et al. (1993) Protein Eng. 6(3):327-331).

In one embodiment, cell based assays can be exploited to analyze a variegated IC47615 library. For example, a library of expression vectors can be transfected into a cell line, e.g., a neuronal cell line, which ordinarily responds to an IC47615 ligand in a particular IC47615 ligand-dependent manner. The transfected cells are then contacted with an IC47615 ligand and the effect of expression of the mutant on, e.g., membrane excitability of IC47615 can be detected. Plasmid DNA can then be recovered from the cells which score for inhibition, or alternatively, potentiation of signaling by the IC47615 ligand, and the individual clones further characterized.

An isolated IC47615 protein, or a portion or fragment thereof, can be used as an immunogen to generate antibodies that bind IC47615 using standard techniques for polyclonal and monoclonal antibody preparation. A full-length IC47615 protein can be used or, alternatively, the invention provides antigenic peptide fragments of IC47615 for use as immunogens. The antigenic peptide of IC47615 comprises at least 8 amino acid residues of the amino acid sequence shown in SEQ ID NO:35 and encompasses an epitope of IC47615 such that an antibody raised against the peptide forms a specific immune complex with IC47615. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of IC47615 that are located on the surface of the protein, e.g., hydrophilic regions, as well as regions with high antigenicity (see FIG. 30).

An IC47615 immunogen typically is used to prepare antibodies by immunizing a suitable subject, (e.g., rabbit, goat, mouse or other mammal) with the immunogen. An appropriate immunogenic preparation can contain, for example, recombinantly expressed IC47615 protein or a chemically synthesized IC47615 polypeptide. The preparation can further include an adjuvant, such as Freund's complete or incomplete adjuvant, or similar immunostimulatory agent. Immunization of a suitable subject with an immunogenic IC47615 preparation induces a polyclonal anti-IC47615 antibody response.

Accordingly, another aspect of the invention pertains to anti-IC47615 antibodies. The term “antibody” as used herein refers to immunoglobulin molecules and immunologically active portions of immunoglobulin molecules, i.e., molecules that contain an antigen binding site which specifically binds (immunoreacts with) an antigen, such as IC47615. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind IC47615. The term “monoclonal antibody” or “monoclonal antibody composition”, as used herein, refers to a population of antibody molecules that contain only one species of an antigen binding site capable of immunoreacting with a particular epitope of IC47615. A monoclonal antibody composition thus typically displays a single binding affinity for a particular IC47615 protein with which it immunoreacts.

Polyclonal anti-IC47615 antibodies can be prepared as described above by immunizing a suitable subject with an IC47615 immunogen. The anti-IC47615 antibody titer in the immunized subject can be monitored over time by standard techniques, such as with an enzyme linked immunosorbent assay (ELISA) using immobilized IC47615. If desired, the antibody molecules directed against IC47615 can be isolated from the mammal (e.g., from the blood) and further purified by well known techniques, such as protein A chromatography to obtain the IgG fraction. At an appropriate time after immunization, e.g., when the anti-IC47615 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally Kenneth, R. H., in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an IC47615 immunogen as described above, and the culture supernatants of the resulting hybridoma cells are screened to identify a hybridoma producing a monoclonal antibody that binds IC47615.

Any of the many well known protocols used for fusing lymphocytes and immortalized cell lines can be applied for the purpose of generating an anti-IC47615 monoclonal antibody (see, e.g., Galfre, G. et al. (1977) Nature 266:55052; Gefter et al. (1977) supra; Lerner (1981) supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind IC47615, e.g., using a standard ELISA assay.

Alternative to preparing monoclonal antibody-secreting hybridomas, a monoclonal anti-IC47615 antibody can be identified and isolated by screening a recombinant combinatorial immunoglobulin library (e.g., an antibody phage display library) with IC47615 to thereby isolate immunoglobulin library members that bind IC47615. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-IC47615 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) i J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) i J. Immunol. 141:4053-4060.

An anti-IC47615 antibody (e.g., monoclonal antibody) can be used to isolate IC47615 by standard techniques, such as affinity chromatography or immunoprecipitation. An anti-IC47615 antibody can facilitate the purification of natural IC47615 from cells and of recombinantly produced IC47615 expressed in host cells. Moreover, an anti-IC47615 antibody can be used to detect IC47615 protein (e.g., in a cellular lysate or cell supernatant) in order to evaluate the abundance and pattern of expression of the IC47615 protein. Anti-IC47615 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding an IC47615 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., IC47615 proteins, mutant forms of IC47615 proteins, fusion proteins, and the like).

The recombinant expression vectors of the invention can be designed for expression of IC47615 proteins in prokaryotic or eukaryotic cells. For example, IC47615 proteins can be expressed in bacterial cells such as E. coli, insect cells (using baculovirus expression vectors) yeast cells or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in IC47615 activity assays, (e.g., direct assays or competitive assays described in detail below), or to generate antibodies specific for IC47615 proteins, for example. In a preferred embodiment, an IC47615 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al. (1988) Gene 69:301-315) and pET 11d (Studier et al. (1990) Methods Enzymol. 185:60-89) (Studier et al. (1990) Methods Enzymol. 185:60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S. (1990) Methods Enzymol. 185:119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the IC47615 expression vector is a yeast expression vector. Examples of vectors for expression in yeast S. cerevisiae include pYepSec1 (Baldari, et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and picZ (Invitrogen Corp, San Diego, Calif.).

Alternatively, IC47615 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to IC47615 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which an IC47615 nucleic acid molecule of the invention is introduced, e.g., an IC47615 nucleic acid molecule within a recombinant expression vector or an IC47615 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an IC47615 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an IC47615 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an IC47615 protein. Accordingly, the invention further provides methods for producing an IC47615 protein using the host cells of the invention. In one embodiment, the method comprises culturing the host cell of the invention (into which a recombinant expression vector encoding an IC47615 protein has been introduced) in a suitable medium such that an IC47615 protein is produced. In another embodiment, the method further comprises isolating an IC47615 protein from the medium or the host cell.

The host cells of the invention can also be used to produce non-human transgenic animals. For example, in one embodiment, a host cell of the invention is a fertilized oocyte or an embryonic stem cell into which IC47615-coding sequences have been introduced. Such host cells can then be used to create non-human transgenic animals in which exogenous IC47615 sequences have been introduced into their genome or homologous recombinant animals in which endogenous IC47615 sequences have been altered. Such animals are useful for studying the function and/or activity of an IC47615 and for identifying and/or evaluating modulators of IC47615 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous IC47615 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an IC47615-encoding nucleic acid into the male pronuclei of a fertilized oocyte, e.g., by microinjection, retroviral infection, and allowing the oocyte to develop in a pseudopregnant female foster animal. The IC47615 cDNA sequence of SEQ ID NO:34 can be introduced as a transgene into the genome of a non-human animal. Alternatively, a nonhuman homologue of a human IC47615 gene, such as a mouse or rat IC47615 gene, can be used as a transgene. Alternatively, an IC47615 gene homologue, such as another IC47615 family member, can be isolated based on hybridization to the IC47615 cDNA sequences of SEQ ID NO:34 or 36, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to an IC47615 transgene to direct expression of an IC47615 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of an IC47615 transgene in its genome and/or expression of IC47615 mRNA in tissues or cells of the animals. A transgenic founder animal can then be used to breed additional animals carrying the transgene. Moreover, transgenic animals carrying a transgene encoding an IC47615 protein can further be bred to other transgenic animals carrying other transgenes.

To create a homologous recombinant animal, a vector is prepared which contains at least a portion of an IC47615 gene into which a deletion, addition or substitution has been introduced to thereby alter, e.g., functionally disrupt, the IC47615 gene. The IC47615 gene can be a human gene (e.g., the cDNA of SEQ ID NO:36), but more preferably, is a non-human homologue of a human IC47615 gene (e.g., a cDNA isolated by stringent hybridization with the nucleotide sequence of SEQ ID NO:34). For example, a mouse IC47615 gene can be used to construct a homologous recombination nucleic acid molecule, e.g., a vector, suitable for altering an endogenous IC47615 gene in the mouse genome. In a preferred embodiment, the homologous recombination nucleic acid molecule is designed such that, upon homologous recombination, the endogenous IC47615 gene is functionally disrupted (i.e., no longer encodes a functional protein; also referred to as a “knock out” vector). Alternatively, the homologous recombination nucleic acid molecule can be designed such that, upon homologous recombination, the endogenous IC47615 gene is mutated or otherwise altered but still encodes functional protein (e.g., the upstream regulatory region can be altered to thereby alter the expression of the endogenous IC47615 protein). In the homologous recombination nucleic acid molecule, the altered portion of the IC47615 gene is flanked at its 5′ and 3′ ends by additional nucleic acid sequence of the IC47615 gene to allow for homologous recombination to occur between the exogenous IC47615 gene carried by the homologous recombination nucleic acid molecule and an endogenous IC47615 gene in a cell, e.g., an embryonic stem cell. The additional flanking IC47615 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced IC47615 gene has homologously recombined with the endogenous IC47615 gene are selected (see e.g., Li, E. et al. (1992) Cell 69:915). The selected cells can then be injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The IC47615 nucleic acid molecules, fragments of IC47615 proteins, and anti-IC47615 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of an IC47615 protein or an anti-IC47615 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, an IC47615 protein of the invention has one or more of the following activities: (1) modulates membrane excitability, (2) regulates intracellular ion concentration, (3) modulates membrane polarization (e.g., membrane polarization and/or depolarization), (4) regulates action potential, (5) regulates cellular signal transduction, (6) regulates neurotransmitter release (e.g., from neuronal cells), (7) modulates synaptic transmission, (8) regulates neuronal excitability and/or plasticity, (9) regulates muscle contraction, (10) regulate cell activation and (11) regulates cellular proliferation, growth, migration and/or differentiation.

The isolated nucleic acid molecules of the invention can be used, for example, to express IC47615 protein (e.g., via a recombinant expression vector in a host cell in gene therapy applications), to detect IC47615 mRNA (e.g., in a biological sample) or a genetic alteration in an IC47615 gene, and to modulate IC47615 activity, as described further below. The IC47615 proteins can be used to treat disorders characterized by insufficient or excessive production of an IC47615 substrate or production of IC47615 inhibitors. In addition, the IC47615 proteins can be used to screen for naturally occurring IC47615 substrates, to screen for drugs or compounds which modulate IC47615 activity, as well as to treat disorders characterized by insufficient or excessive production of IC47615 protein or production of IC47615 protein forms which have decreased, aberrant or unwanted activity compared to IC47615 wild type protein (e.g., proliferative disorders, CNS disorders such as cognitive and neurodegenerative disorders (e.g., Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, and bipolar affective disorder (e.g., severe bipolar affective (mood) disorder (BP-1) and bipolar affective neurological disorders (e.g., migraine and obesity)); pain disorders; cardiac disorders (e.g., arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia), muscular disorders (e.g., paralysis, muscle weakness (e.g., ataxia, myotonia, and myokymia), muscular dystrophy (e.g., Duchenne muscular dystrophy or myotonic dystrophy), spinal muscular atrophy, congenital myopathies, central core disease, rod myopathy, central nuclear myopathy, Lambert-Eaton syndrome, denervation, and infantile spinal muscular atrophy (Werdnig-Hoffman disease), and cellular growth, differentiation, or migration disorders (e.g., cancer, e.g., carcinoma, sarcoma, or leukemia; tumor angiogenesis and metastasis; skeletal dysplasia; neuronal deficiencies resulting from impaired neural induction and patterning; hepatic disorders; cardiovascular disorders; and hematopoietic and/or myeloproliferative disorders). Moreover, the anti-IC47615 antibodies of the invention can be used to detect and isolate IC47615 proteins, regulate the bioavailability of IC47615 proteins, and modulate IC47615 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to IC47615 proteins, have a stimulatory or inhibitory effect on, for example, IC47615 expression or IC47615 activity, or have a stimulatory or inhibitory effect on, for example, the expression or activity of IC47615 substrate.

In one embodiment, the invention provides assays for screening candidate or test compounds which are substrates of an IC47615 protein or polypeptide or biologically active portion thereof. In another embodiment, the invention provides assays for screening candidate or test compounds which bind to or modulate the activity of an IC47615 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g, Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, an assay is a cell-based assay in which a cell which expresses an IC47615 protein or biologically active portion thereof is contacted with a test compound and the ability of the test compound to modulate IC47615 activity is determined. Determining the ability of the test compound to modulate IC47615 activity can be accomplished by monitoring, for example, the release of a neurotransmitter from a cell which expresses IC47615. The cell, for example, can be of mammalian origin, e.g., a neuronal cell or a thymus cell. The ability of the test compound to modulate IC47615 binding to a substrate or to bind to IC47615 can also be determined. Determining the ability of the test compound to modulate IC47615 binding to a substrate can be accomplished, for example, by coupling the IC47615 substrate with a radioisotope or enzymatic label such that binding of the IC47615 substrate to IC47615 can be determined by detecting the labeled IC47615 substrate in a complex. Alternatively, IC47615 could be coupled with a radioisotope or enzymatic label to monitor the ability of a test compound to modulate IC47615 binding to an IC47615 substrate in a complex. Determining the ability of the test compound to bind IC47615 can be accomplished, for example, by coupling the compound with a radioisotope or enzymatic label such that binding of the compound to IC47615 can be determined by detecting the labeled IC47615 compound in a complex. For example, compounds (e.g., IC47615 substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., an IC47615 substrate) to interact with IC47615 without the labeling of any of the interactants. For example, a microphysiometer can be used to detect the interaction of a compound with IC47615 without the labeling of either the compound or the IC47615. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and IC47615.

In another embodiment, an assay is a cell-based assay comprising contacting a cell expressing an IC47615 target molecule (e.g., an IC47615 substrate) with a test compound and determining the ability of the test compound to modulate (e.g., stimulate or inhibit) the activity of the IC47615 target molecule. Determining the ability of the test compound to modulate the activity of an IC47615 target molecule can be accomplished, for example, by determining the ability of the IC47615 protein to bind to or interact with the IC47615 target molecule.

Determining the ability of the IC47615 protein, or a biologically active fragment thereof, to bind to or interact with an IC47615 target molecule can be accomplished by one of the methods described above for determining direct binding. In a preferred embodiment, determining the ability of the IC47615 protein to bind to or interact with an IC47615 target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target using an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which an IC47615 protein or biologically active portion thereof is contacted with a test compound and the ability of the test compound to bind to the IC47615 protein or biologically active portion thereof is determined. Preferred biologically active portions of the IC47615 proteins to be used in assays of the present invention include fragments which participate in interactions with non-IC47615 molecules, e.g., fragments with high surface probability scores (see, for example, FIG. 30). Binding of the test compound to the IC47615 protein can be determined either directly or indirectly as described above. In a preferred embodiment, the assay includes contacting the IC47615 protein or biologically active portion thereof with a known compound which binds IC47615 to form an assay mixture, contacting the assay mixture with a test compound, and determining the ability of the test compound to interact with an IC47615 protein, wherein determining the ability of the test compound to interact with an IC47615 protein comprises determining the ability of the test compound to preferentially bind to IC47615 or biologically active portion thereof as compared to the known compound.

In another embodiment, the assay is a cell-free assay in which an IC47615 protein or biologically active portion thereof is contacted with a test compound and the ability of the test compound to modulate (e.g., stimulate or inhibit) the activity of the IC47615 protein or biologically active portion thereof is determined. Determining the ability of the test compound to modulate the activity of an IC47615 protein can be accomplished, for example, by determining the ability of the IC47615 protein to bind to an IC47615 target molecule by one of the methods described above for determining direct binding. Determining the ability of the IC47615 protein to bind to an IC47615 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an IC47615 protein can be accomplished by determining the ability of the IC47615 protein to further modulate the activity of a downstream effector of an IC47615 target molecule. For example, the activity of the effector molecule on an appropriate target can be determined or the binding of the effector to an appropriate target can be determined as previously described.

In yet another embodiment, the cell-free assay involves contacting an IC47615 protein or biologically active portion thereof with a known compound which binds the IC47615 protein to form an assay mixture, contacting the assay mixture with a test compound, and determining the ability of the test compound to interact with the IC47615 protein, wherein determining the ability of the test compound to interact with the IC47615 protein comprises determining the ability of the IC47615protein to preferentially bind to or modulate the activity of an IC47615 target molecule.

In more than one embodiment of the above assay methods of the present invention, it may be desirable to immobilize either IC47615 or its target molecule to facilitate separation of complexed from uncomplexed forms of one or both of the proteins, as well as to accommodate automation of the assay. Binding of a test compound to an IC47615 protein, or interaction of an IC47615 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/IC47615 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or IC47615 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of IC47615 binding or activity determined using standard techniques.

Other techniques for immobilizing proteins on matrices can also be used in the screening assays of the invention. For example, either an IC47615 protein or an IC47615 target molecule can be immobilized utilizing conjugation of biotin and streptavidin. Biotinylated IC47615 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with IC47615 protein or target molecules but which do not interfere with binding of the IC47615 protein to its target molecule can be derivatized to the wells of the plate, and unbound target or IC47615 protein trapped in the wells by antibody conjugation. Methods for detecting such complexes, in addition to those described above for the GST-immobilized complexes, include immunodetection of complexes using antibodies reactive with the IC47615 protein or target molecule, as well as enzyme-linked assays which rely on detecting an enzymatic activity associated with the IC47615 protein or target molecule.

In another embodiment, modulators of IC47615 expression are identified in a method wherein a cell is contacted with a candidate compound and the expression of IC47615 mRNA or protein in the cell is determined. The level of expression of IC47615 mRNA or protein in the presence of the candidate compound is compared to the level of expression of IC47615 mRNA or protein in the absence of the candidate compound. The candidate compound can then be identified as a modulator of IC47615 expression based on this comparison. For example, when expression of IC47615 mRNA or protein is greater (statistically significantly greater) in the presence of the candidate compound than in its absence, the candidate compound is identified as a stimulator of IC47615 mRNA or protein expression. Alternatively, when expression of IC47615 mRNA or protein is less (statistically significantly less) in the presence of the candidate compound than in its absence, the candidate compound is identified as an inhibitor of IC47615 mRNA or protein expression. The level of IC47615 mRNA or protein expression in the cells can be determined by methods described herein for detecting IC47615 mRNA or protein.

In yet another aspect of the invention, the IC47615 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with IC47615 (“IC47615-binding proteins” or “IC47615-bp”) and are involved in IC47615 activity. Such IC47615-binding proteins are also likely to be involved in the propagation of signals by the IC47615 proteins or IC47615 targets as, for example, downstream elements of an IC47615-mediated signaling pathway. Alternatively, such IC47615-binding proteins are likely to be IC47615 inhibitors.

The two-hybrid system is based on the modular nature of most transcription factors, which consist of separable DNA-binding and activation domains. Briefly, the assay utilizes two different DNA constructs. In one construct, the gene that codes for an IC47615 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an IC47615-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the IC47615 protein.

In another aspect, the invention pertains to a combination of two or more of the assays described herein. For example, a modulating agent can be identified using a cell-based or a cell free assay, and the ability of the agent to modulate the activity of an IC47615 protein can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., an IC47615 modulating agent, an antisense IC47615 nucleic acid molecule, an IC47615-specific antibody, or an IC47615-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the IC47615 nucleotide sequences, described herein, can be used to map the location of the IC47615 genes on a chromosome. The mapping of the IC47615 sequences to chromosomes is an important first step in correlating these sequences with genes associated with disease.

Briefly, IC47615 genes can be mapped to chromosomes by preparing PCR primers (preferably 15-25 bp in length) from the IC47615 nucleotide sequences. Computer analysis of the IC47615 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the IC47615 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the IC47615 nucleotide sequences to design oligonucleotide primers, sublocalization can be achieved with panels of fragments from specific chromosomes. Other mapping strategies which can similarly be used to map an IC47615 sequence to its chromosome include in situ hybridization (described in Fan, Y. et al. (1990) Proc. Natl. Acad. Sci. USA 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al, Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the IC47615 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The IC47615 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the IC47615 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The IC47615 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:34 can comfortably provide positive individual identification with a panel of perhaps 10 to 1,000 primers which each yield a noncoding amplified sequence of 100 bases. If predicted coding sequences, such as that in SEQ ID NO:36 are used, a more appropriate number of primers for positive individual identification would be 500-2,000.

If a panel of reagents from IC47615 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of IC47615 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:34 are particularly appropriate for this use as greater numbers of polymorphisms occur in the noncoding regions, making it easier to differentiate individuals using this technique. Examples of polynucleotide reagents include the IC47615 nucleotide sequences or portions thereof, e.g., fragments derived from the noncoding regions of SEQ ID NO:34 having a length of at least 20 bases, preferably at least 30 bases.

The IC47615 nucleotide sequences described herein can further be used to provide polynucleotide reagents, e.g., labeled or labelable probes which can be used in, for example, an in situ hybridization technique, to identify a specific tissue, e.g., thymus or brain tissue. This can be very useful in cases where a forensic pathologist is presented with a tissue of unknown origin. Panels of such IC47615 probes can be used to identify tissue by species and/or by organ type.

In a similar fashion, these reagents, e.g., IC47615 primers or probes can be used to screen tissue culture for contamination (i.e., screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining IC47615 protein and/or nucleic acid expression as well as IC47615 activity, in the context of a biological sample (e.g., blood, serum, cells, tissue) to thereby determine whether an individual is afflicted with a disease or disorder, or is at risk of developing a disorder, associated with aberrant or unwanted IC47615 expression or activity. The invention also provides for prognostic (or predictive) assays for determining whether an individual is at risk of developing a disorder associated with IC47615 protein, nucleic acid expression or activity. For example, mutations in an IC47615 gene can be assayed in a biological sample. Such assays can be used for prognostic or predictive purpose to thereby prophylactically treat an individual prior to the onset of a disorder characterized by or associated with IC47615 protein, nucleic acid expression or activity.

Another aspect of the invention pertains to monitoring the influence of agents (e.g., drugs, compounds) on the expression or activity of IC47615 in clinical trials.

These and other agents are described in further detail in the following sections.

1. Diagnostic Assays

An exemplary method for detecting the presence or absence of IC47615 protein or nucleic acid in a biological sample involves obtaining a biological sample from a test subject and contacting the biological sample with a compound or an agent capable of detecting IC47615 protein or nucleic acid (e.g., mRNA, or genomic DNA) that encodes IC47615 protein such that the presence of IC47615 protein or nucleic acid is detected in the biological sample. A preferred agent for detecting IC47615 mRNA or genomic DNA is a labeled nucleic acid probe capable of hybridizing to IC47615 mRNA or genomic DNA. The nucleic acid probe can be, for example, the IC47615 nucleic acid set forth in SEQ ID NO:34 or 36, or the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to IC47615 mRNA or genomic DNA. Other suitable probes for use in the diagnostic assays of the invention are described herein.

A preferred agent for detecting IC47615 protein is an antibody capable of binding to IC47615 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect IC47615 mRNA, protein, or genomic DNA in a biological sample in vitro as well as in vivo. For example, in vitro techniques for detection of IC47615 mRNA include Northern hybridizations and in situ hybridizations. In vitro techniques for detection of IC47615 protein include enzyme linked immunosorbent assays (ELISAs), Western blots, immunoprecipitations and immunofluorescence. In vitro techniques for detection of IC47615 genomic DNA include Southern hybridizations. Furthermore, in vivo techniques for detection of IC47615 protein include introducing into a subject a labeled anti-IC47615 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting IC47615 protein, mRNA, or genomic DNA, such that the presence of IC47615 protein, mRNA or genomic DNA is detected in the biological sample, and comparing the presence of IC47615 protein, mRNA or genomic DNA in the control sample with the presence of IC47615 protein, mRNA or genomic DNA in the test sample.

The invention also encompasses kits for detecting the presence of IC47615 in a biological sample. For example, the kit can comprise a labeled compound or agent capable of detecting IC47615 protein or mRNA in a biological sample; means for determining the amount of IC47615 in the sample; and means for comparing the amount of IC47615 in the sample with a standard. The compound or agent can be packaged in a suitable container. The kit can further comprise instructions for using the kit to detect IC47615 protein or nucleic acid.

2. Prognostic Assays

The diagnostic methods described herein can furthermore be utilized to identify subjects having or at risk of developing a disease or disorder associated with aberrant or unwanted IC47615 expression or activity. As used herein, the term “aberrant” includes an IC47615 expression or activity which deviates from the wild type IC47615 expression or activity. Aberrant expression or activity includes increased or decreased expression or activity, as well as expression or activity which does not follow the wild type developmental pattern of expression or the subcellular pattern of expression. For example, aberrant IC47615 expression or activity is intended to include the cases in which a mutation in the IC47615 gene causes the IC47615 gene to be under-expressed or over-expressed and situations in which such mutations result in a non-functional IC47615 protein or a protein which does not function in a wild-type fashion, e.g., a protein which does not interact with an IC47615 substrate, e.g., a non-IC47615 channel subunit or ligand, or one which interacts with a non-IC47615 substrate, e.g. a non-IC47615 channel subunit or ligand. As used herein, the term “unwanted” includes an unwanted phenomenon involved in a biological response such as cellular proliferation. For example, the term unwanted includes an IC47615 expression or activity which is undesirable in a subject.

The assays described herein, such as the preceding diagnostic assays or the following assays, can be utilized to identify a subject having or at risk of developing a disorder associated with a misregulation in IC47615 protein activity or nucleic acid expression, such as a CNS disorder (e.g., a cognitive or neurodegenerative disorder), a pain disorder, a muscular disorder, a cellular proliferation, growth, differentiation, or migration disorder, or a cardiovascular disorder. Alternatively, the prognostic assays can be utilized to identify a subject having or at risk for developing a disorder associated with a misregulation in IC47615 protein activity or nucleic acid expression, such as a CNS disorder, a pain disorder, a cellular proliferation, growth, differentiation, or migration disorder, a muscular disorder, or a cardiovascular disorder. Thus, the present invention provides a method for identifying a disease or disorder associated with aberrant or unwanted IC47615 expression or activity in which a test sample is obtained from a subject and IC47615 protein or nucleic acid (e.g., mRNA or genomic DNA) is detected, wherein the presence of IC47615 protein or nucleic acid is diagnostic for a subject having or at risk of developing a disease or disorder associated with aberrant or unwanted IC47615 expression or activity. As used herein, a “test sample” refers to a biological sample obtained from a subject of interest. For example, a test sample can be a biological fluid (e.g., cerebrospinal fluid or serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted IC47615 expression or activity. For example, such methods can be used to determine whether a subject can be effectively treated with an agent for a CNS disorder, a pain disorder, a muscular disorder, a cardiovascular disorder, or a cellular proliferation, growth, differentiation, or migration disorder. Thus, the present invention provides methods for determining whether a subject can be effectively treated with an agent for a disorder associated with aberrant or unwanted IC47615 expression or activity in which a test sample is obtained and IC47615 protein or nucleic acid expression or activity is detected (e.g., wherein the abundance of IC47615 protein or nucleic acid expression or activity is diagnostic for a subject that can be administered the agent to treat a disorder associated with aberrant or unwanted IC47615 expression or activity).

The methods of the invention can also be used to detect genetic alterations in an IC47615 gene, thereby determining if a subject with the altered gene is at risk for a disorder characterized by misregulation in IC47615 protein activity or nucleic acid expression, such as a CNS disorder, a pain disorder, a cellular proliferation, growth, differentiation, or migration disorder, a muscular disorder, or cardiovascular disorder. In preferred embodiments, the methods include detecting, in a sample of cells from the subject, the presence or absence of a genetic alteration characterized by at least one of an alteration affecting the integrity of a gene encoding an IC47615-protein, or the mis-expression of the IC47615 gene. For example, such genetic alterations can be detected by ascertaining the existence of at least one of 1) a deletion of one or more nucleotides from an IC47615 gene; 2) an addition of one or more nucleotides to an IC47615 gene; 3) a substitution of one or more nucleotides of an IC47615 gene, 4) a chromosomal rearrangement of an IC47615 gene; 5) an alteration in the level of a messenger RNA transcript of an IC47615 gene, 6) aberrant modification of an IC47615 gene, such as of the methylation pattern of the genomic DNA, 7) the presence of a non-wild type splicing pattern of a messenger RNA transcript of an IC47615 gene, 8) a non-wild type level of an IC47615-protein, 9) allelic loss of an IC47615 gene, and 10) inappropriate post-translational modification of an IC47615-protein. As described herein, there are a large number of assays known in the art which can be used for detecting alterations in an IC47615 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the IC47615-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to an IC47615 gene under conditions such that hybridization and amplification of the IC47615-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an IC47615 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in IC47615 can be identified by hybridizing a sample and control nucleic acids, e.g., DNA or RNA, to high density arrays containing hundreds or thousands of oligonucleotides probes (Cronin, M. T. et al. (1996) Hum. Mutat. 7:244-255; Kozal, M. J. et al. (1996) Nat. Med. 2:753-759). For example, genetic mutations in IC47615 can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the IC47615 gene and detect mutations by comparing the sequence of the sample IC47615 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the IC47615 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type IC47615 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in IC47615 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an IC47615 sequence, e.g., a wild-type IC47615 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in IC47615 genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat. Res. 285:125-144; and Hayashi (1992) Genet. Anal. Tech. Appl. 9:73-79). Single-stranded DNA fragments of sample and control IC47615 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an IC47615 gene.

Furthermore, any cell type or tissue in which IC47615 is expressed may be utilized in the prognostic assays described herein.

3. Monitoring of Effects During Clinical Trials

Monitoring the influence of agents (e.g., drugs) on the expression or activity of an IC47615 protein (e.g., the modulation of cell proliferation and/or migration) can be applied not only in basic drug screening, but also in clinical trials. For example, the effectiveness of an agent determined by a screening assay as described herein to increase IC47615 gene expression, protein levels, or upregulate IC47615 activity, can be monitored in clinical trials of subjects exhibiting decreased IC47615 gene expression, protein levels, or downregulated IC47615 activity. Alternatively, the effectiveness of an agent determined by a screening assay to decrease IC47615 gene expression, protein levels, or downregulate IC47615 activity, can be monitored in clinical trials of subjects exhibiting increased IC47615 gene expression, protein levels, or upregulated IC47615 activity. In such clinical trials, the expression or activity of an IC47615 gene, and preferably, other genes that have been implicated in, for example, an IC47615-associated disorder can be used as a “read out” or markers of the phenotype of a particular cell.

For example, and not by way of limitation, genes, including IC47615, that are modulated in cells by treatment with an agent (e.g., compound, drug or small molecule) which modulates IC47615 activity (e.g., identified in a screening assay as described herein) can be identified. Thus, to study the effect of agents on IC47615-associated disorders (e.g., disorders characterized by deregulated cell proliferation and/or migration), for example, in a clinical trial, cells can be isolated and RNA prepared and analyzed for the levels of expression of IC47615 and other genes implicated in the IC47615-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of IC47615 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an IC47615 protein, mRNA, or genomic DNA in the preadministration sample; (iii) obtaining one or more post-administration samples from the subject; (iv) detecting the level of expression or activity of the IC47615 protein, mRNA, or genomic DNA in the post-administration samples; (v) comparing the level of expression or activity of the IC47615 protein, mRNA, or genomic DNA in the pre-administration sample with the IC47615 protein, mRNA, or genomic DNA in the post administration sample or samples; and (vi) altering the administration of the agent to the subject accordingly. For example, increased administration of the agent may be desirable to increase the expression or activity of IC47615 to higher levels than detected, i.e., to increase the effectiveness of the agent. Alternatively, decreased administration of the agent may be desirable to decrease expression or activity of IC47615 to lower levels than detected, i.e., to decrease the effectiveness of the agent. According to such an embodiment, IC47615 expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted IC47615 expression or activity, e.g., a CNS disorder, a pain disorder, a cellular proliferation, growth, differentiation, or migration disorder, a muscular disorder, or a cardiovascular disorder. As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose to cure, heal, alleviate, relieve, alter, remedy, ameliorate, improve, or affect the disease or disorder, the symptom of the disease or disorder, or the risk of (or susceptibility to) the disease or disorder. As used herein, a “therapeutic agent” includes, but is not limited to, small molecules, peptides, polypeptides, antibodies, ribozymes, and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the IC47615 molecules of the present invention or IC47615 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted IC47615 expression or activity, by administering to the subject an IC47615 or an agent which modulates IC47615 expression or at least one IC47615 activity. Subjects at risk for a disease which is caused or contributed to by aberrant or unwanted IC47615 expression or activity can be identified by, for example, any or a combination of diagnostic or prognostic assays as described herein. Administration of a prophylactic agent can occur prior to the manifestation of symptoms characteristic of the IC47615 aberrancy, such that a disease or disorder is prevented or, alternatively, delayed in its progression. Depending on the type of IC47615 aberrancy, for example, an IC47615, IC47615 agonist or IC47615 antagonist agent can be used for treating the subject. The appropriate agent can be determined based on screening assays described herein.

2. Therapeutic Methods

Another aspect of the invention pertains to methods of modulating IC47615 expression or activity for therapeutic purposes. Accordingly, in an exemplary embodiment, the modulatory method of the invention involves contacting a cell with an IC47615 or agent that modulates one or more of the activities of IC47615 protein activity associated with the cell. An agent that modulates IC47615 protein activity can be an agent as described herein, such as a nucleic acid or a protein, a naturally-occurring target molecule of an IC47615 protein (e.g., an IC47615 substrate), an IC47615 antibody, an IC47615 agonist or antagonist, a peptidomimetic of an IC47615 agonist or antagonist, or other small molecule. In one embodiment, the agent stimulates one or more IC47615 activities. Examples of such stimulatory agents include active IC47615 protein and a nucleic acid molecule encoding IC47615 that has been introduced into the cell. In another embodiment, the agent inhibits one or more IC47615 activities. Examples of such inhibitory agents include antisense IC47615 nucleic acid molecules, anti-IC47615 antibodies, and IC47615 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of an IC47615 protein or nucleic acid molecule. In one embodiment, the method involves administering an agent (e.g., an agent identified by a screening assay described herein), or combination of agents that modulates (e.g., upregulates or downregulates) IC47615 expression or activity. In another embodiment, the method involves administering an IC47615 protein or nucleic acid molecule as therapy to compensate for reduced, aberrant, or unwanted IC47615 expression or activity.

Stimulation of IC47615 activity is desirable in situations in which IC47615 is abnormally downregulated and/or in which increased IC47615 activity is likely to have a beneficial effect. Likewise, inhibition of IC47615 activity is desirable in situations in which IC47615 is abnormally upregulated and/or in which decreased IC47615 activity is likely to have a beneficial effect.

3. Pharmacogenomics

The IC47615 molecules of the present invention, as well as agents, or modulators which have a stimulatory or inhibitory effect on IC47615 activity (e.g., IC47615 gene expression) as identified by a screening assay described herein can be administered to individuals to treat (prophylactically or therapeutically) IC47615-associated disorders (e.g., proliferative disorders) associated with aberrant or unwanted IC47615 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an IC47615 molecule or IC47615 modulator as well as tailoring the dosage and/or therapeutic regimen of treatment with an IC47615 molecule or IC47615 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., an IC47615 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme is the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an IC47615 molecule or IC47615 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an IC47615 molecule or IC47615 modulator, such as a modulator identified by one of the exemplary screening assays described herein.

E. Electronic Apparatus Readable Media and Arrays

Electronic apparatus readable media comprising IC47615 sequence information is also provided. As used herein, “IC47615 sequence information” refers to any nucleotide and/or amino acid sequence information particular to the IC47615 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said IC47615 sequence information includes detection of the presence or absence of a sequence (eg., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding, or containing data or information that can be read and accessed directly by an electronic apparatus. Such media can include, but are not limited to: magnetic storage media, such as floppy discs, hard disc storage medium, and magnetic tape; optical storage media such as compact discs; electronic storage media such as RAM, ROM, EPROM, EEPROM and the like; and general hard disks and hybrids of these categories such as magnetic/optical storage media. The medium is adapted or configured for having recorded thereon IC47615 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatuses; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the IC47615 sequence information.

A variety of software programs and formats can be used to store the sequence information on the electronic apparatus readable medium. For example, the sequence information can be represented in a word processing text file, formatted in commercially-available software such as WordPerfect and Microsoft Word, represented in the form of an ASCII file, or stored in a database application, such as DB2, Sybase, Oracle, or the like, as well as in other forms. Any number of dataprocessor structuring formats (e.g., text file or database) may be employed in order to obtain or create a medium having recorded thereon the IC47615 sequence information.

By providing IC47615 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has an IC47615 associated disease or disorder or a pre-disposition to an IC47615 associated disease or disorder, wherein the method comprises the steps of determining IC47615 sequence information associated with the subject and based on the IC47615 sequence information, determining whether the subject has an IC47615 associated disease or disorder or a pre-disposition to an IC47615 associated disease or disorder, and/or recommending a particular treatment for the disease, disorder, or pre-disease condition.

The present invention further provides in an electronic system and/or in a network, a method for determining whether a subject has an IC47615 associated disease or disorder or a pre-disposition to a disease associated with IC47615 wherein the method comprises the steps of determining IC47615 sequence information associated with the subject, and based on the IC47615 sequence information, determining whether the subject has an IC47615 associated disease or disorder or a pre-disposition to an IC47615 associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has an IC47615 associated disease or disorder or a pre-disposition to an IC47615 associated disease or disorder associated with IC47615, said method comprising the steps of receiving IC47615 sequence information from the subject and/or information related thereto, receiving phenotypic information associated with the subject, acquiring information from the network corresponding to IC47615 and/or an IC47615 associated disease or disorder, and based on one or more of the phenotypic information, the IC47615 information (e.g., sequence information and/or information related thereto), and the acquired information, determining whether the subject has an IC47615 associated disease or disorder or a pre-disposition to an IC47615 associated disease or disorder. The method may further comprise the step of recommending a particular treatment for the disease, disorder or pre-disease condition.

The present invention also provides a business method for determining whether a subject has an IC47615 associated disease or disorder or a pre-disposition to an IC47615 associated disease or disorder, said method comprising the steps of receiving information related to IC47615 (e.g., sequence information and/or information related thereto), receiving phenotypic information associated with the subject, acquiring information from the network related to IC47615 and/or related to an IC47615 associated disease or disorder, and based on one or more of the phenotypic information, the IC47615 information, and the acquired information, determining whether the subject has an IC47615 associated disease or disorder or a pre-disposition to an IC47615 associated disease or disorder. The method may further comprise the step of recommending a particular treatment for the disease, disorder or pre-disease condition.

The invention also includes an array comprising an IC47615 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be IC47615. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of an IC47615 associated disease or disorder, progression of IC47615 associated disease or disorder, and processes, such a cellular transformation associated with the IC47615 associated disease or disorder.

The array is also useful for ascertaining the effect of the expression of a gene on the expression of other genes in the same cell or in different cells (e.g., ascertaining the effect of IC47615 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including IC47615) that could serve as a molecular target for diagnosis or therapeutic intervention.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human IC47615 cDNA

In this example, the identification and characterization of the gene encoding human IC47615 (clone Fbh47615FL) is described.

Isolation of the IC47615 cDNA

The invention is based, at least in part, on the discovery of a human gene encoding a novel protein, referred to herein as IC47615. The entire sequence of the human clone Fbh47615FL was determined and found to contain an open reading frame termed human “IC47615.” The nucleotide sequence of the human IC47615 gene is set forth in FIGS. 29A-C and in SEQ ID NOs:34 and 35. The amino acid sequence of the human IC47615 expression product is set forth in FIG. 29 and in SEQ ID NO:35.

The nucleotide sequence encoding the human IC47615 protein is shown in FIGS. 29A-C and is set forth as SEQ ID NO:34. The protein encoded by this nucleic acid comprises about 305 amino acids and has the amino acid sequence shown in FIGS. 29A-C and set forth as SEQ ID NO:35. The coding region (open reading frame) of SEQ ID NO:34 is set forth as SEQ ID NO:36. Clone Fbh47615FL, comprising the coding region of human IC47615, was deposited with the American Type Culture Collection (ATCC®), 10801 University Boulevard, Manassas, Va. 20110-2209, on ______, and assigned Accession No. ______.

Analysis of the Human IC47615 Molecules

The amino acid sequence of human IC47615 was analyzed using the program PSORT (http://www.psort.nibb.ac.jp) to predict the localization of the proteins within the cell. This program assesses the presence of different targeting and localization amino acid sequences within the query sequence. The results of the analysis show that human IC47615 (SEQ ID NO:35) may be localized to the mitochondrion, to the cytoplasm, or to the nucleus.

A search was performed against the Memsat database (FIG. 31), resulting in the identification of a transmembrane domain in the amino acid sequence of human IC47615 (SEQ ID NO:35) at about residues 274-290.

Tissue Distribution of IC47615 mRNA

This example describes the tissue distribution of IC47615 mRNA, as is determined by Polymerase Chain Reaction (PCR) on cDNA libraries using oligonucleotide primers based on the human IC47615 sequence.

For in situ analysis, various tissues, e.g., tissues obtained from brain, are first frozen on dry ice. Ten-micrometer-thick sections of the tissues are postfixed with 4% formaldehyde in DEPC treated 1×phosphate-buffered saline at room temperature for 10 minutes before being rinsed twice in DEPC 1×phosphate-buffered saline and once in 0.1 M triethanolamine-HCl (pH 8.0). Following incubation in 0.25% acetic anhydride-0.1 M triethanolamine-HCl for 10 minutes, sections are rinsed in DEPC 2×SSC (1×SSC is 0.15M NaCl plus 0.015M sodium citrate). Tissue is then dehydrated through a series of ethanol washes, incubated in 100% chloroform for 5 minutes, and then rinsed in 100% ethanol for 1 minute and 95% ethanol for 1 minute and allowed to air dry.

Hybridizations are performed with ³⁵S-radiolabeled (5×10⁷ cpm/ml) cRNA probes. Probes are incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides are washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides are then rinsed with 2×SSC at room temperature, washed with 2×SSC at 50° C. for 1 hour, washed with 0.2×SSC at 55° C. for 1 hour, and 0.2×SSC at 60° C. for 1 hour. Sections are then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

### Example 2

### Expression of Recombinant IC47615 Protein in Bacterial Cells

In this example, IC47615 is expressed as a recombinant glutathione-S-transferase (GST) fusion polypeptide in E. coli and the fusion polypeptide is isolated and characterized. Specifically, IC47615 is fused to GST and this fusion polypeptide is expressed in E. coli, e.g., strain PEB199. Expression of the GST-IC47615 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant IC47615 Protein in COS Cells

To express the IC47615 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire IC47615 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the IC47615 DNA sequence is amplified by PCR using two primers. The 5′ primer contains the restriction site of interest followed by approximately twenty nucleotides of the IC47615 coding sequence starting from the initiation codon; the 3′ end sequence contains complementary sequences to the other restriction site of interest, a translation stop codon, the HA tag or FLAG tag and the last 20 nucleotides of the IC47615 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the IC47615 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the IC47615-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J. et al., Molecular Cloning: A Laboratory Manual. 2nd, ed., Cold Spring Harbor Laboratory, Cold Spring Harbor Laboratory Press, Cold Spring Harbor, N.Y., 1989. The expression of the IC47615 polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the IC47615 coding sequence is cloned directly into the polylinker of the pCDNA/Amp vector using the appropriate restriction sites. The resulting plasmid is transfected into COS cells in the manner described above, and the expression of the IC47615 polypeptide is detected by radiolabeling and immunoprecipitation using an IC47615 specific monoclonal antibody.

### VII. 55063, A NOVEL HUMAN NMDA FAMILY MEMBER AND USES THEREOF

### Background of the Invention

Ion channels constitute a large family of membrane-bound proteins responsible for a wide range of important transport and signaling functions in cells. Members of this family regulate ion selectivity in response to a specific stimulus such as a change in voltage across a biological membrane (voltage-gated channels), a mechanical stress (mechanically gated channels, or the binding of a ligand (ligand-gated channels). lonotropic neurotransmitter receptors (or transmitter-gated ion channels) are members of the ligand-gated ion channel family and include receptors with specificity for acetylcholine, serotonin, glycine, glutamate, and GABA (γ-aminobutyric acid). Transmitter-gated ion channels rapidly convert extracellular chemical signals into electrical signals at chemical synapses. The channels open transiently in response to neurotransmitter binding, producing a transient permeability change in the membrane, and resulting in a change in the membrane potential.

Glutamate-gated ion channels are members of the transmitter-gated ion channel family. These receptors exist as multimeric proteins (typically pentamers) which may consist of various combinations of monomeric subunits, thus, generating a large array of receptors with varying channel subtypes, ligand affinities, channel conductances, opening and closing rates, and different sensitivities to drugs and toxins (Bigge (1999) Curr. Op. Chem. Biol. 3:441-447). The glutamate-gated ion channel family includes three classes of receptors: kainate receptors, AMPA (α-amino-3-hydroxy-5-methyl-4-isoxazolepropionic acid) receptors, and NMDA (N-methyl-D-aspartic acid) receptors. The NMDA receptors control intracellular Ca²⁺ concentration in response to the binding of the agonist L-glutamate and the co-agonist glycine, and initiates long-term potentiation at synapses (Yamakura et al. (1999) Prog. Neurobiol. 59:279-298).

NMDA receptors are glycosylated proteins that contain four transmembrane regions, a large amino-terminal cytoplasmic domain, and a small carboxy-terminal intracellular domain which contains phosphorylation sites responsible for modifying receptor activity (Bigge (1999) Curr. Op. Chem. Biol. 3:441-447). In situ hybridization in the mouse has shown a ubiquitous distribution of NMDA receptors in the brain, while NMDA mRNA is observed in characteristic distributions in the brain and spinal cord, and is differentially expressed during development in the brain (Yamakura et al. (1999) Prog. Neurobiol. 59:279-298). These receptors are molecularly diverse, with multiple subunits displaying distinct distributions, properties and regulation, depending upon the region of the brain in which they are expressed, as well as the developmental stage at which they are expressed. They play a role in synaptic plasticity and synapse formation underlying memory, learning, and formation of neural networks during development. Pathological states involving NMDA receptors include acute and chronic neurological disorders; psychiatric disorders; and neuropathic pain syndromes.

### Summary of the Invention

The present invention is based, at least in part, on the discovery of novel human N-methyl-D-aspartate (NMDA) family members, referred to herein as “human NMDA-1” or “HNMDA-1” nucleic acid and polypeptide molecules. The HNMDA-1 nucleic acid and polypeptide molecules of the present invention are useful as modulating agents in regulating a variety of cellular processes, e.g., synaptic plasticity and synapse formation. Accordingly, in one aspect, this invention provides isolated nucleic acid molecules encoding HNMDA-1 polypeptides or biologically active portions thereof, as well as nucleic acid fragments suitable as primers or hybridization probes for the detection of HNMDA-1-encoding nucleic acids.

In one embodiment, the invention features an isolated nucleic acid molecule that includes the nucleotide sequence set forth in SEQ ID NO:37 or 39. In another embodiment, the invention features an isolated nucleic acid molecule that encodes a polypeptide including the amino acid sequence set forth in SEQ ID NO:38. In another embodiment, the invention features an isolated nucleic acid molecule that includes the nucleotide sequence contained in the plasmid deposited with ATCC® as Accession Number ______.

In still other embodiments, the invention features isolated nucleic acid molecules including nucleotide sequences that are substantially identical (e.g., 71.2%, 72%, 75%, 80%, 85%, 90%, 95%, 96%, 97%, 98% or 99% identical) to the nucleotide sequence set forth as SEQ ID NO:37 or 39. The invention further features isolated nucleic acid molecules including at least 591 contiguous nucleotides of the nucleotide sequence set forth as SEQ ID NO:37 or 39. In another embodiment, the invention features isolated nucleic acid molecules which encode a polypeptide including an amino acid sequence that is substantially identical (e.g., 94.2%, 95%, 96%, 97%, 98%, or 99% identical) to the amino acid sequence set forth as SEQ ID NO:38. The present invention also features nucleic acid molecules which encode allelic variants of the polypeptide having the amino acid sequence set forth as SEQ ID NO:38. In addition to isolated nucleic acid molecules encoding full-length polypeptides, the present invention also features nucleic acid molecules which encode fragments, for example, biologically active or antigenic fragments, of the full-length polypeptides of the present invention (e.g., fragments including at least 97 contiguous amino acid residues of the amino acid sequence of SEQ ID NO:38). In still other embodiments, the invention features nucleic acid molecules that are complementary to, antisense to, or hybridize under stringent conditions to the isolated nucleic acid molecules described herein.

In another aspect, the invention provides vectors including the isolated nucleic acid molecules described herein (e.g., HNMDA-1-encoding nucleic acid molecules). Such vectors can optionally include nucleotide sequences encoding heterologous polypeptides. Also featured are host cells including such vectors (e.g., host cells including vectors suitable for producing HNMDA-1 nucleic acid molecules and polypeptides).

In another aspect, the invention features isolated HNMDA-1 polypeptides and/or biologically active or antigenic fragments thereof. Exemplary embodiments feature a polypeptide including the amino acid sequence set forth as SEQ ID NO:38, a polypeptide including an amino acid sequence at least 94.2%, 95%, 96%, 97%, 98%, or 99% identical to the amino acid sequence set forth as SEQ ID NO:38, a polypeptide encoded by a nucleic acid molecule including a nucleotide sequence at least 71.2%, 72%, 75%, 80%, 85%, 90%, 95%, 96%, 97%, 98% or 99% identical to the nucleotide sequence set forth as SEQ ID NO:37 or 39. Also featured are fragments of the full-length polypeptides described herein (e.g., fragments including at least 97 contiguous amino acid residues of the sequence set forth as SEQ ID NO:38) as well as allelic variants of the polypeptide having the amino acid sequence set forth as SEQ ID NO:38.

The HNMDA-1 polypeptides and/or biologically active or antigenic fragments thereof, are useful, for example, as reagents or targets in assays applicable to treatment and/or diagnosis of HNMDA-1 mediated or related disorders. In one embodiment, an HNMDA-1 polypeptide or fragment thereof, has an HNMDA-1 activity. In another embodiment, an HNMDA-1 polypeptide or fragment thereof, includes a transmembrane domain, a signal sequence, an ionotropic glutamate receptor family domain, and/or a ligand-gated ion channel family domain, and optionally, has an HNMDA-1 activity. In a related aspect, the invention features antibodies (e.g., antibodies which specifically bind to any one of the polypeptides described herein) as well as fusion polypeptides including all or a fragment of a polypeptide described herein.

The present invention further features methods for detecting HNMDA-1 polypeptides and/or HNMDA-1 nucleic acid molecules, such methods featuring, for example, a probe, primer or antibody described herein. Also featured are kits e.g., kits for the detection of HNMDA-1 polypeptides and/or HNMDA-1 nucleic acid molecules. In a related aspect, the invention features methods for identifying compounds which bind to and/or modulate the activity of an HNMDA-1 polypeptide or HNMDA-1 nucleic acid molecule described herein. Further featured are methods for modulating an HNMDA-1 activity.

Other features and advantages of the invention will be apparent from the following detailed description and claims.

### Detailed Description of the Invention

The present invention is based, at least in part, on the discovery of novel molecules, referred to herein as “human NMDA-1” or “HNMDA-1” nucleic acid and polypeptide molecules, which are novel members of the glutamate-gated ion channel family. These novel molecules are capable of, for example, modulating a glutamate-gated ion channel mediated activity (e.g., an NMDA mediated activity) in a neural cell (e.g., in the brain and/or spinal cord). These novel molecules are capable of binding neurotransmitters, e.g., L-glutamate and glycine, and transporting ions, e.g., Ca²⁺, across neural membranes and, thus, play a role in or function in a variety of cellular processes, e.g., mediating excitatory postsynaptic currents (e.g., long term potentiation).

As used herein, a “glutamate-gated ion channel” includes a protein or polypeptide which is a member of the ligand-gated ion channel family and is involved in binding ligands, (e.g., binding L-glutamate and glycine), and transporting ions (e.g., Ca²⁺) across the plasma membrane of a cell (e.g., a neural cell). Glutamate-gated ion channels regulate long term potentiation in a cell and, typically, have glutamate substrate specificity. Examples of glutamate-gated ion channels include kainate, AMPA, and NMDA receptors.

As used herein, a “glutamate-gated ion channel mediated activity” includes an activity which involves a glutamate-gated ion channel in a cell (e.g., in a neural cell). Glutamate-gated ion channel mediated activities include the binding of a ligand (e.g., L-glutamine and/or glycine); the transporting of Ca²⁺ across a neural membrane; the regulation of long term potentiation; and the regulation of synapse formation underlying memory, learning, and formation of neural networks during development.

As the HNMDA-1 molecules of the present invention are glutamate-gated ion channels, they may be useful for developing novel diagnostic and therapeutic agents for glutamate-gated ion channel associated disorders. As used herein, the term “glutamate-gated ion channel associated disorder” includes a disorder, disease, or condition which is characterized by an aberrant, e.g., upregulated or downregulated, glutamate-gated ion channel mediated activity. Glutamate-gated ion channel associated disorders typically result in, e.g., upregulated or downregulated, Ca²⁺ levels in a cell (e.g., a neural cell). Examples of glutamate-gated ion channel associated disorders include disorders associated with long term synapse potentiation, acute and chronic neurological disorders, psychiatric disorders, and neuropathic pain syndromes. Glutamate-gated ion channel associated disorders can detrimentally affect conveyance of sensory impulses from the periphery to the brain and/or conductance of motor impulses from the brain to the periphery; integration of reflexes; interpretation of sensory impulses; and emotional, intellectual (e.g., learning and memory), or motor processes.

The term “family” when referring to the polypeptide and nucleic acid molecules of the invention is intended to mean two or more polypeptides or nucleic acid molecules having a common structural domain or motif and having sufficient amino acid or nucleotide sequence homology as defined herein. Such family members can be naturally or non-naturally occurring and can be from either the same or different species. For example, a family can contain a first polypeptide of human origin, as well as other, distinct polypeptides of human origin or alternatively, can contain homologues of non-human origin, e.g., mouse or monkey polypeptides. Members of a family may also have common functional characteristics.

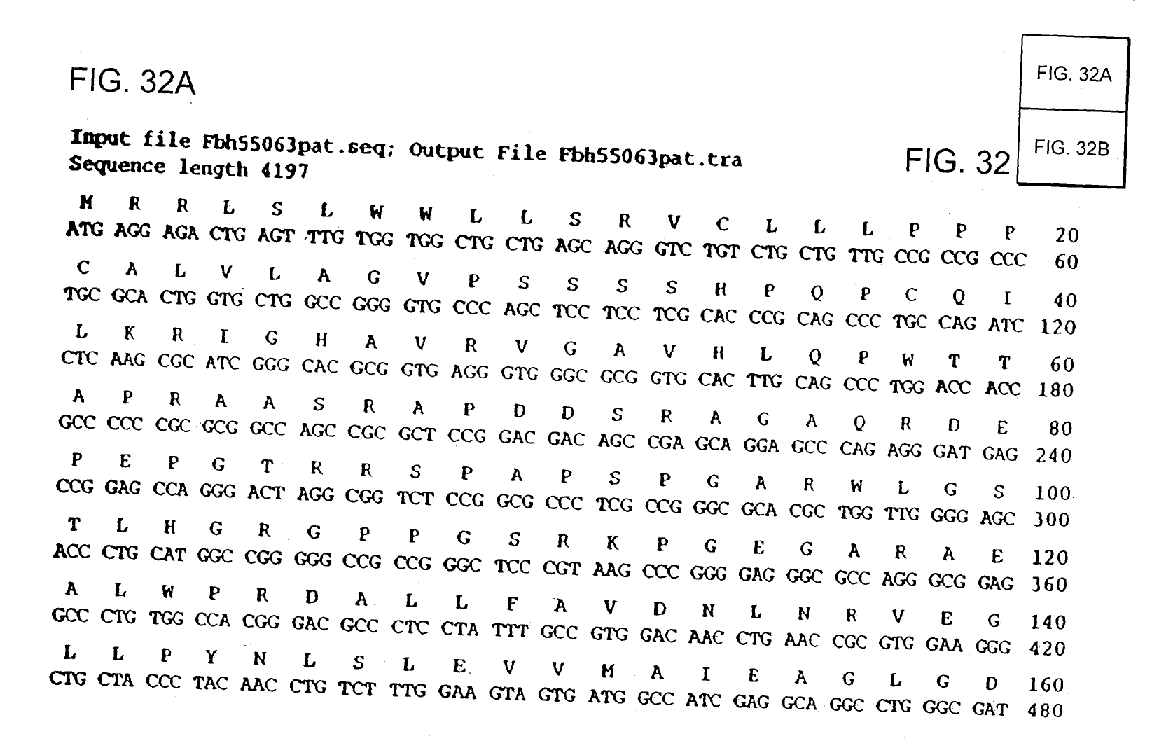

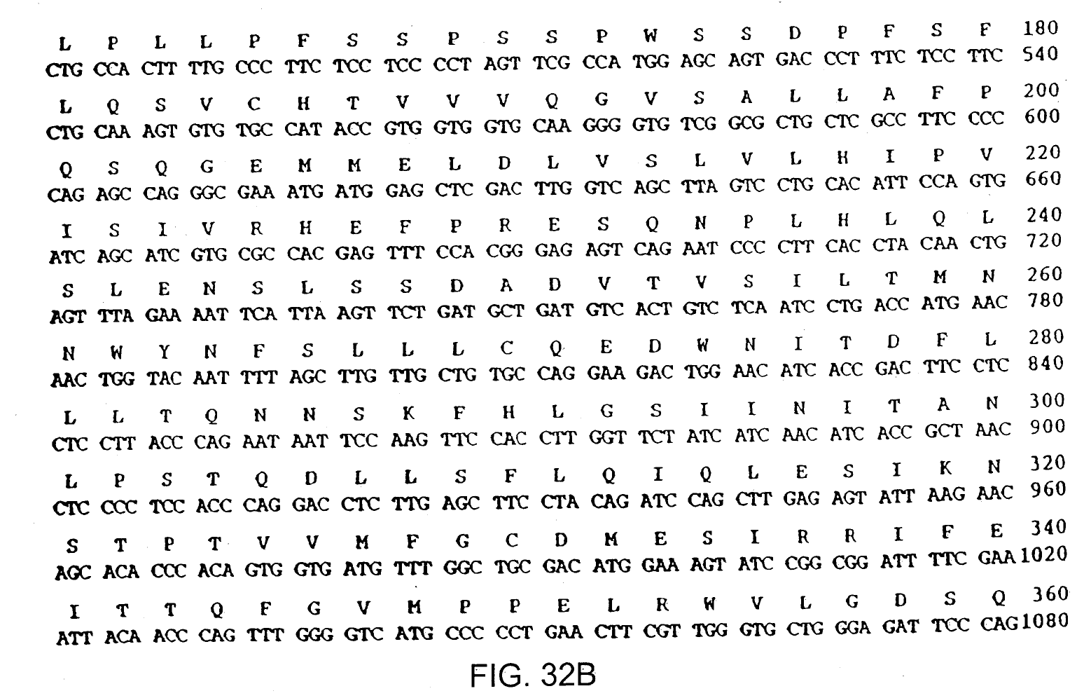

For example, the family of HNMDA-1 polypeptides comprise at least one “transmembrane domain” and preferably four transmembrane domains. As used herein, the term “transmembrane domain” includes an amino acid sequence of about 20-45 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, alanines, valines, phenylalanines, prolines or methionines. Transmembrane domains are described in, for example, Zagotta W. N. et al, (1996) Annual Rev. Neurosci. 19: 235-263, the contents of which are incorporated herein by reference. A MEMSAT analysis resulted in the identification of four transmembrane domains in the amino acid sequence of HNMDA-1 (SEQ ID NO:38) at about residues 7-28, 677-695, 748-770, and 931-951 as set forth in FIG. 32.

Accordingly, HNMDA-1 polypeptides having at least 50-60% homology, preferably about 60-70%, more preferably about 70-80%, or about 80-90% homology with a transmembrane domain of human HNMDA-1 are within the scope of the invention.

In another embodiment of the invention features HNMDA-1 molecules which contain a signal sequence. As used herein, a “signal peptide” includes a peptide of at least about 20 amino acid residues in length which occurs at the N-terminus of secretory and integral membrane proteins and which contains at least 55% hydrophobic amino acid residues. In a preferred embodiment, a signal sequence contains at least about 15-45 amino acid residues, preferably about 20-42 amino acid residues. Signal sequences of 25-35 amino acid residues and 28-32 amino acid residues are also within the scope of the invention. As used herein, a signal sequence has at least about 40-70%, preferably about 50-65%, and more preferably about 55-60% hydrophobic amino acid residues (e.g., Alanine, Valine, Leucine, Isoleucine, Phenylalanine, Tyrosine, Tryptophan, or Proline). Such a “signal peptide”, also referred to in the art as a “signal sequence”, serves to direct a protein containing such a sequence to a lipid bilayer. For example, a signal sequence can be found at about amino acids 1-22 of SEQ ID NO:38 (Met1 to Ala22 of the HNMDA-1 amino acid sequence).

Accordingly, HNMDA-1 polypeptides having at least 50-60% homology, preferably about 60-70%, more preferably about 70-80%, or about 80-90% homology with a signal sequence domain of HNMDA-1 are within the scope of the invention.

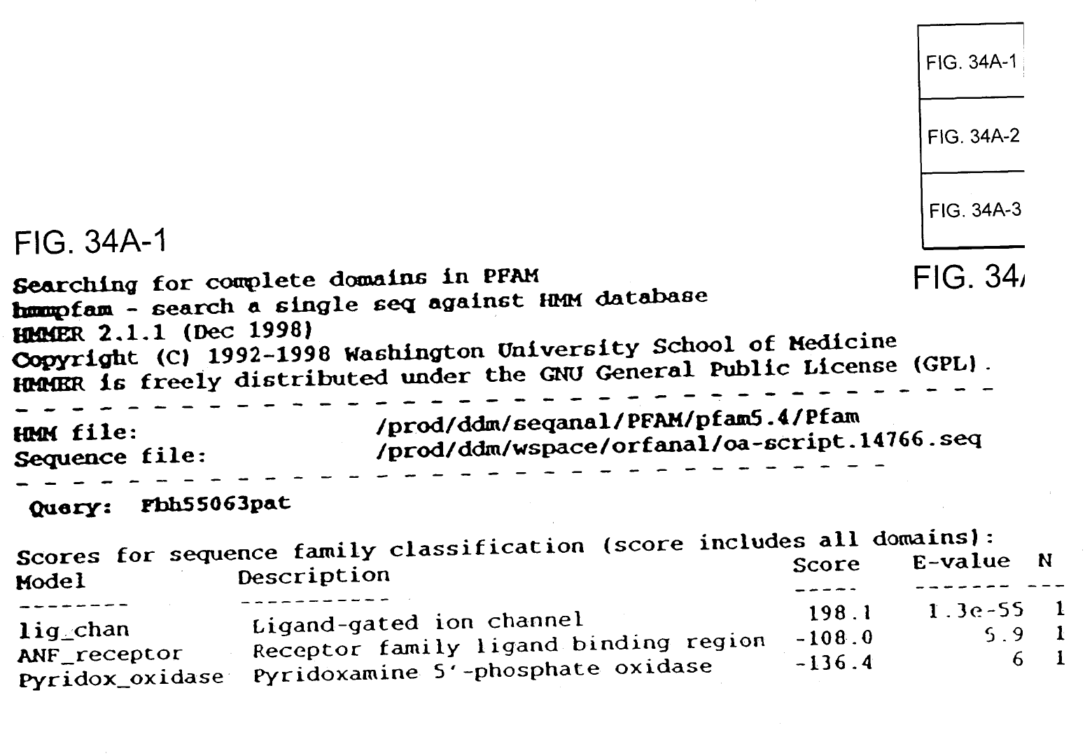

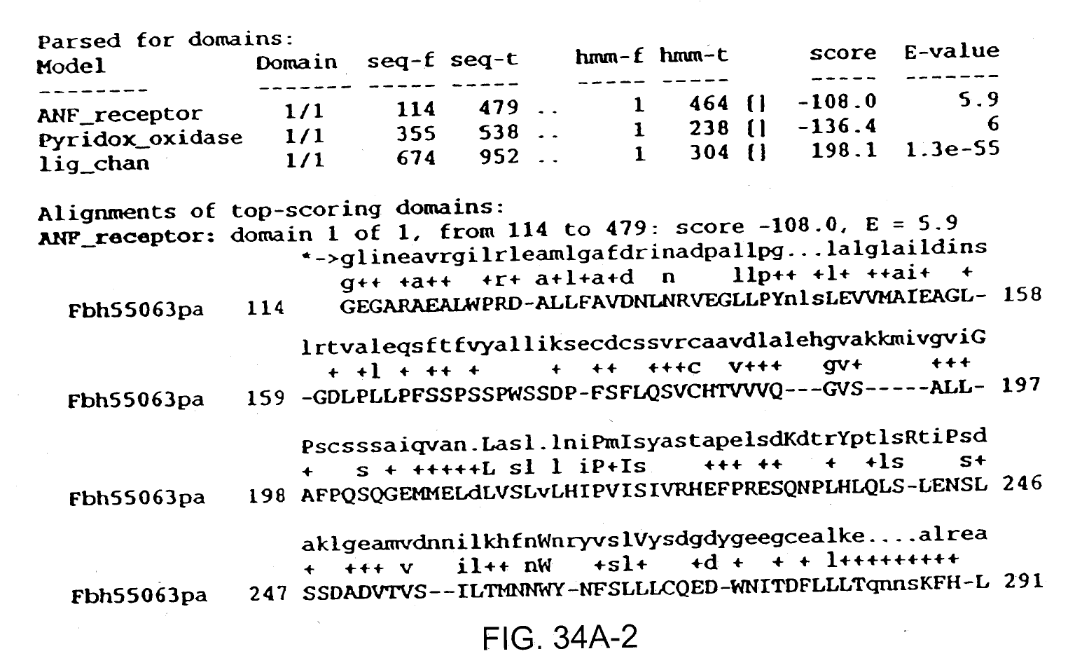

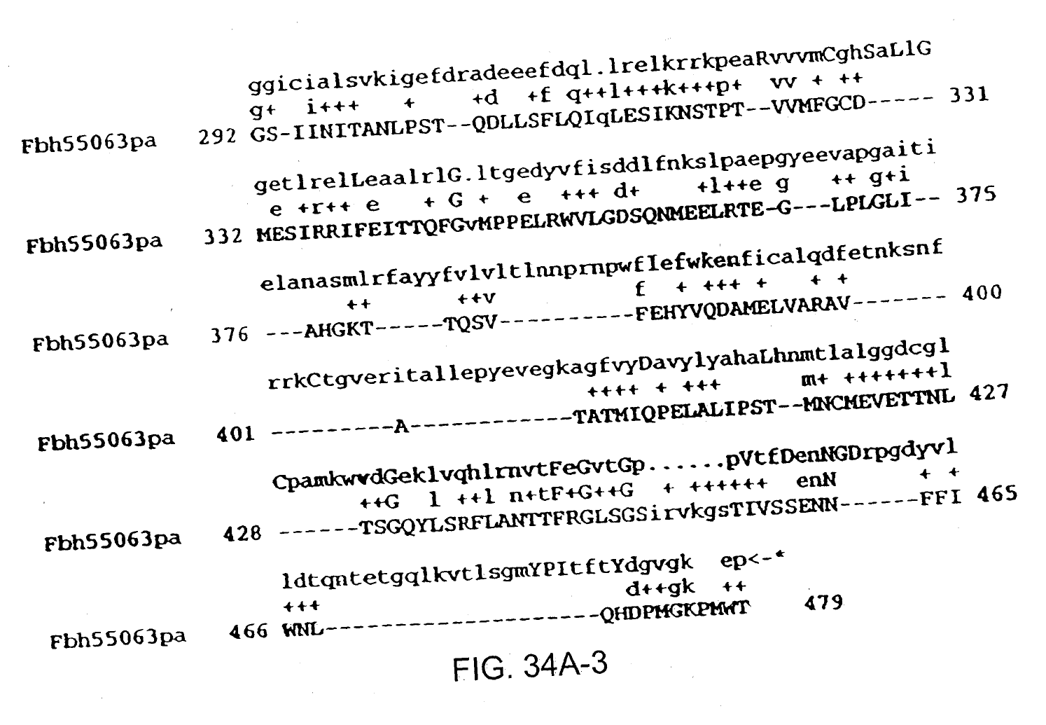

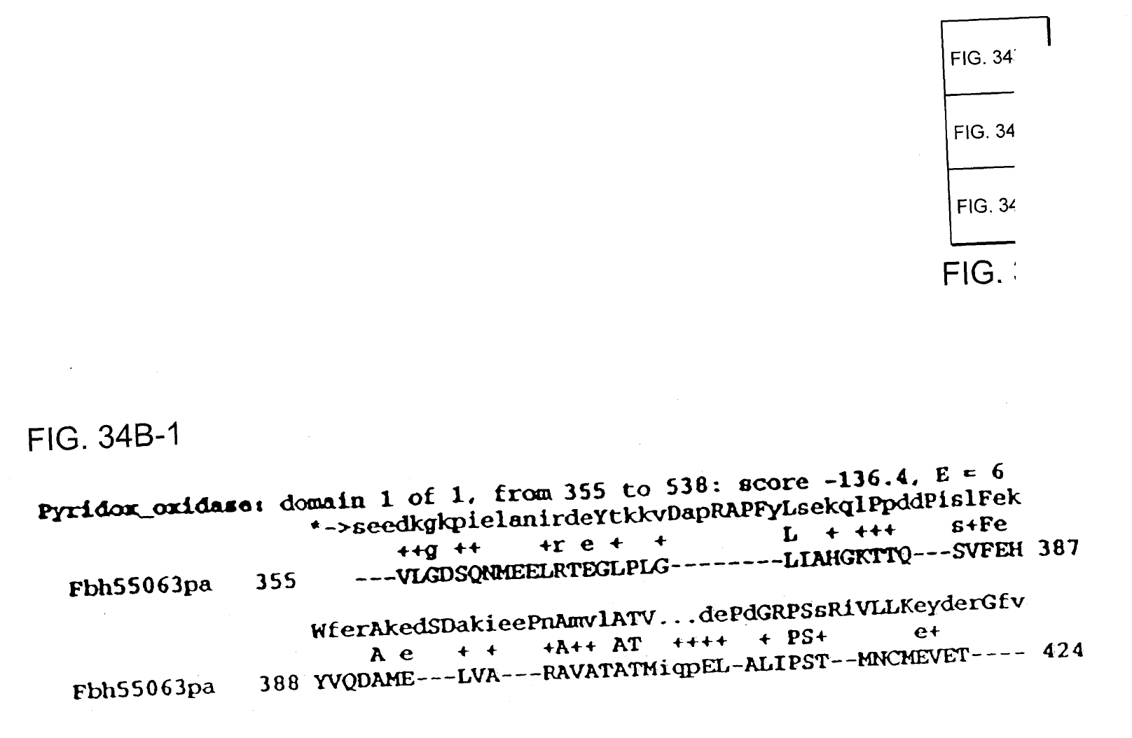

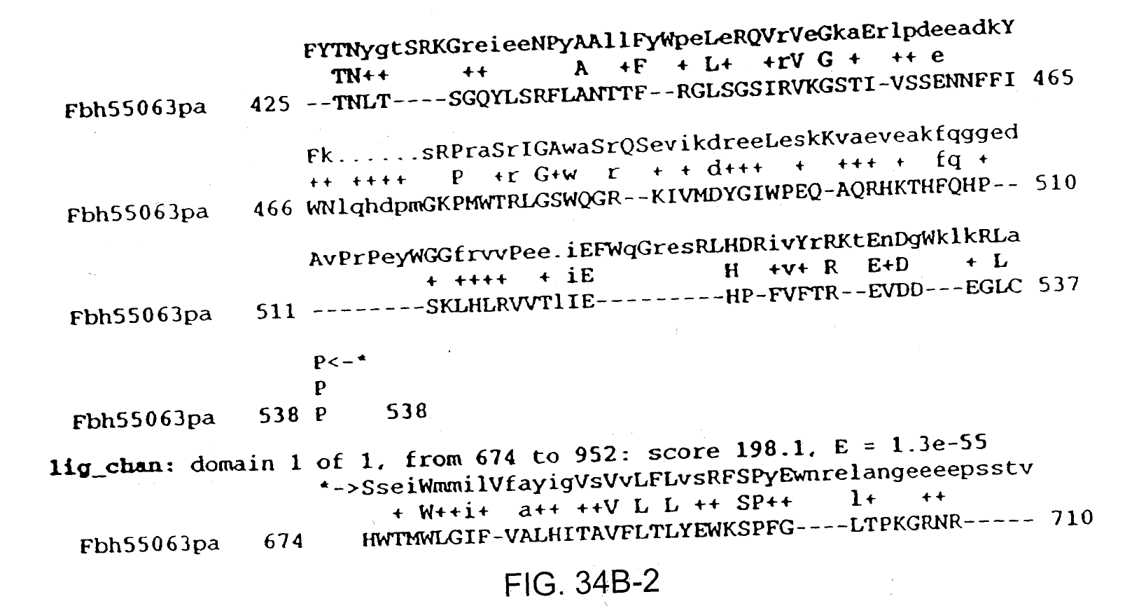

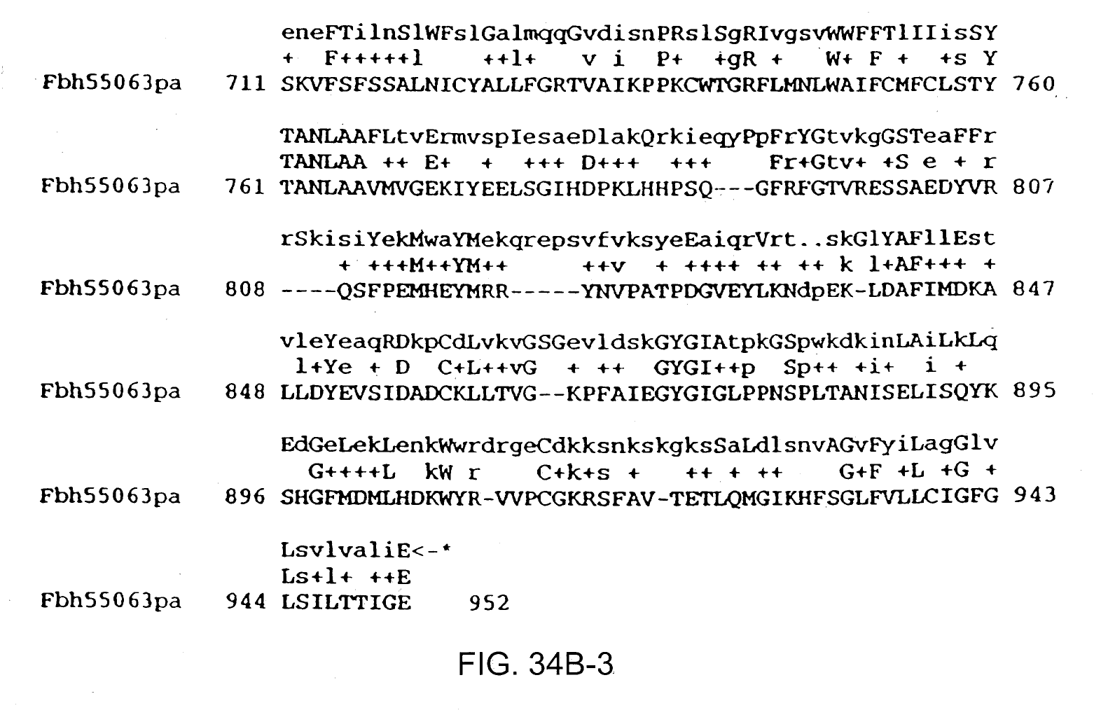

In another embodiment, an HNMDA-1 molecule of the present invention is identified based on the presence of at least one “ligand-gated ion channel family domain.” As used herein, the term “ligand-gated ion channel family domain” includes a protein domain having at least about 200-400 amino acid residues, having a bit score of at least 100 when compared against a ligand-gated ion channel family domain Hidden Markov Model (HMM), and, preferably, a ligand-gated ion channel mediated activity. Preferably, a ligand-gated ion channel family domain includes a polypeptide having an amino acid sequence of about 250-400, 250-350, or more preferably, about 278 amino acid residues, a bit score of at least 160, 170, 180, 190, or more preferably about 198.1, and, preferably a ligand-gated ion channel mediated activity. To identify the presence of a ligand-gated ion channel family domain in an HNMDA-1 protein, and make the determination that a protein of interest has a particular profile, the amino acid sequence of the protein may be searched against a database of known protein domains (e.g., the PFAM HMM database). A PFAM ligand-gated ion channel family domain has been assigned the PFAM Accession PF00060. A search was performed against the PFAM HMM database resulting in the identification of a ligand-gated ion channel family domain in the amino acid sequence of an HNMDA-1 (SEQ ID NO:38) at about residues 674-952 of SEQ ID NO:38. The results of the search are set forth in FIG. 34.

Preferably a “ligand-gated ion channel family domain” has a “ligand-gated ion channel mediated activity” as described herein. For example, a ligand-gated ion channel family domain may have the ability to bind a ligand, e.g., a neurotransmitter (e.g., acetylcholine, serotonin, glycine, glutamate, and/or GABA), on a cell (e.g., a neural cell); and the ability to regulate ion transport in a cell (e.g., Ca²⁺, K⁺, H⁺, Cl⁻, Mg²⁺ and/or Na⁺). Accordingly, identifying the presence of a “ligand-gated ion channel family domain” can include isolating a fragment of an HNMDA-1 molecule (e.g., an HNMDA-1 polypeptide) and assaying for the ability of the fragment to exhibit one of the aforementioned ligand-gated ion channel mediated activities.

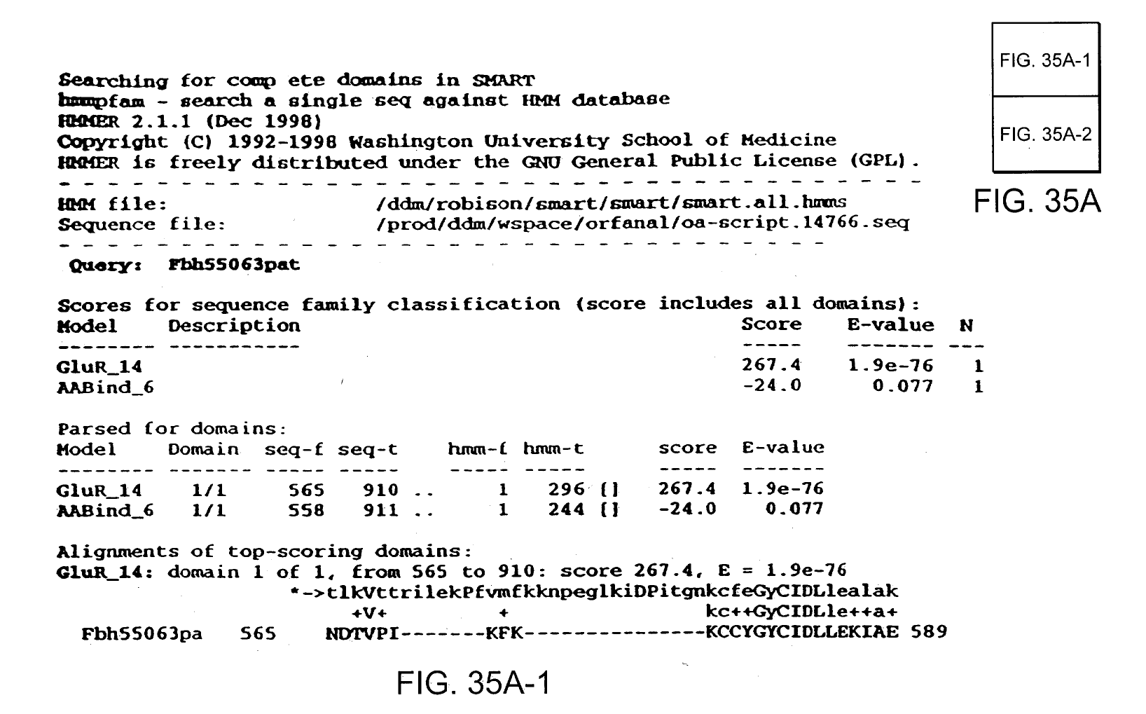

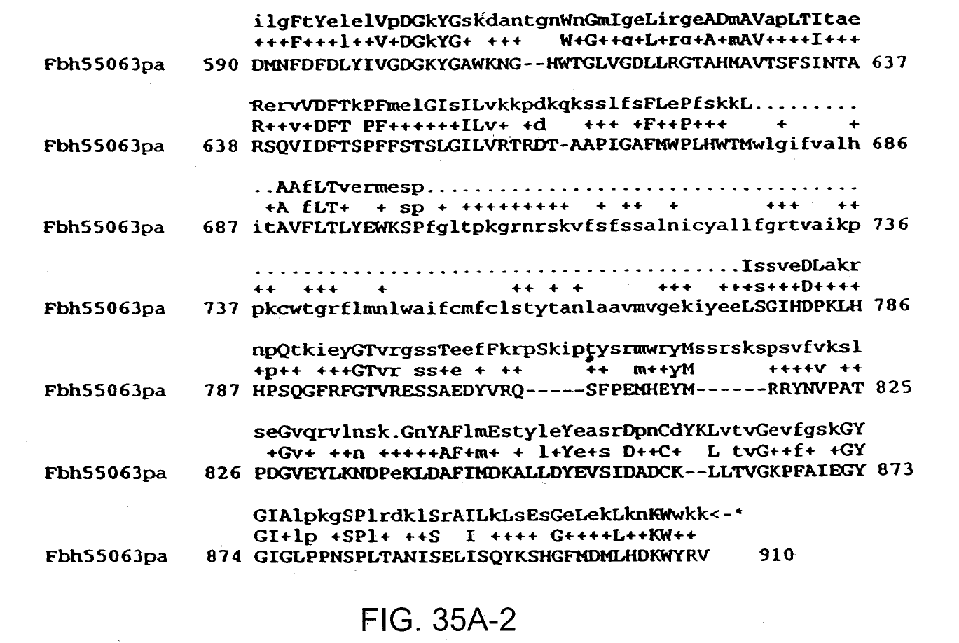

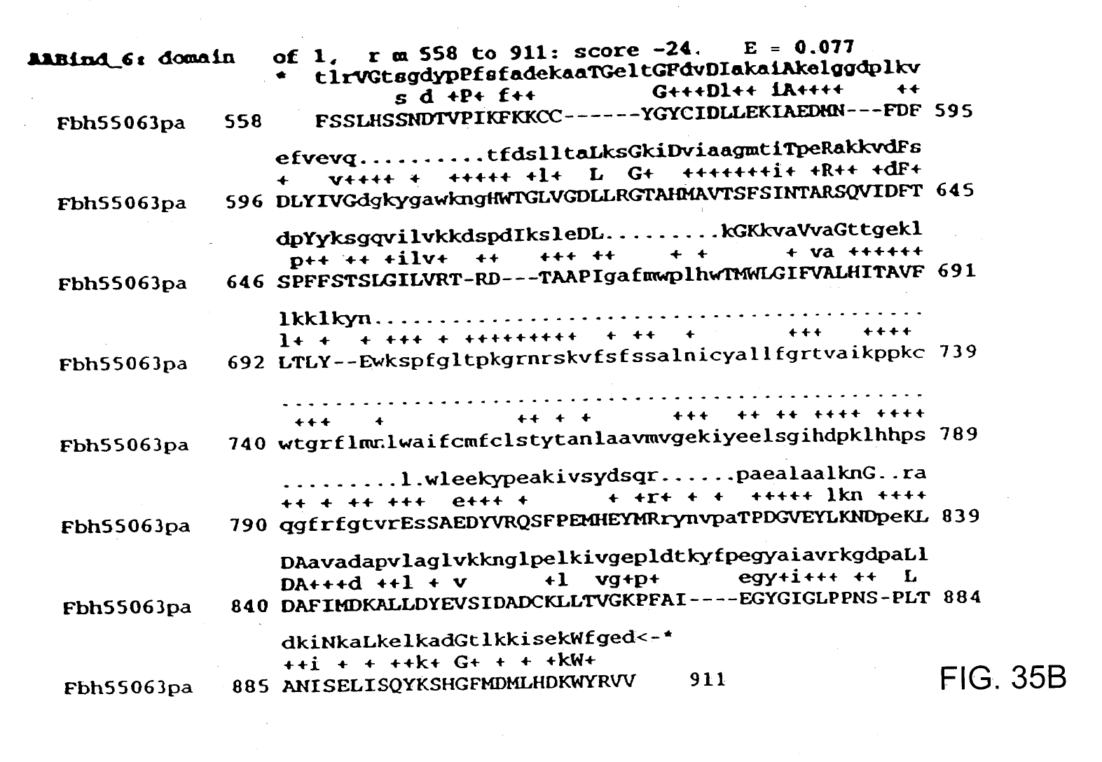

In another embodiment, an HNMDA-1 molecule of the present invention is identified based on the presence of at least one “glutamate-gated ion channel family domain.” As used herein, the term “glutamate-gated ion channel family domain,” also known as an “ionotropic glutamate receptor family domain,” includes a protein domain having at least about 200-500 amino acid residues, having a bit score of at least 200 when compared against a glutamate-gated ion channel family domain Hidden Markov Model (HMM), and a glutamate-gated ion channel mediated activity. Preferably, a glutamate-gated ion channel family domain includes a polypeptide having an amino acid sequence of about 250-450, 300-400, 325-375, or more preferably, about 345 amino acid residues, a bit score of at least 210, 220, 230, 240, 250, 260, or more preferably about 267.4, and a glutamate-gated ion channel mediated activity. To identify the presence of a glutamate-gated ion channel family domain in an HNMDA-1 protein, and make the determination that a protein of interest has a particular profile, the amino acid sequence of the protein may be searched against a database of known protein domains (e.g., the PFAM HMM database). A PFAM glutamate-gated ion channel family domain has been assigned the InterPro Accession IPR001320. A search was performed against the PFAM HMM database resulting in the identification of a glutamate-gated ion channel family domain in the amino acid sequence of an HNMDA-1 (SEQ ID NO:38) at about residues 565-910 of SEQ ID NO:38. The results of the search are set forth in FIGS. 35A-B.

Preferably a “glutamate-gated ion channel family domain” has a “glutamate-gated ion channel mediated activity” as described herein. For example, a glutamate-gated ion channel family domain may have the ability to bind a ligand, e.g., L-glutamate and/or glycine, on a cell (e.g., a neural cell); and the ability to regulate Ca²⁺ transport in a cell. Accordingly, identifying the presence of a “glutamate-gated ion channel family domain” can include isolating a fragment of an HNMDA-1 molecule (e.g., an HNMDA-1 polypeptide) and assaying for the ability of the fragment to exhibit one of the aforementioned glutamate-gated ion channel mediated activities.

A description of the Pfam database can be found in Sonhammer et al. (1997) Proteins 28:405-420 and a detailed description of HMMs can be found, for example, in Gribskov et al.(1990) Meth. Enzymol. 183:146-159; Gribskov et al.(1987) Proc. Natl. Acad, Sci. USA 84:4355-4358; Krogh et al.(1994) J. Mol. Biol. 235:1501-1531; and Stultz et al.(1993) Protein Sci. 2:305-314, the contents of which are incorporated herein by reference.

In a preferred embodiment, the HNMDA-1 molecules of the invention include at least one, preferably two, more preferably three, and even more preferably four transmembrane domain(s) and at least one of the following domains: a signal peptide, a ligand-gated ion channel family domain, and/or a glutamate-gated ion channel family domain.

Isolated HNMDA-1 polypeptides of the present invention have an amino acid sequence sufficiently identical to the amino acid sequence of SEQ ID NO:38 or are encoded by a nucleotide sequence sufficiently identical to SEQ ID NO:37 or 39. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains having at least 50%, 55%, 60%, 65%, 70%, 75%, 80%, 85%, 85%, 90%, 95%, 96%, 97%, 98%, 99% or more homology or identity across the amino acid sequences of the domains and contain at least one and preferably two structural domains or motifs, are defined herein as sufficiently identical. Furthermore, amino acid or nucleotide sequences which share at least 50%, 55%, 60%, 65%, 70%, 75%, 80%, 85%, 85%, 90%, 95%, 96%, 97%, 98%, 99% or more homology or identity and share a common functional activity are defined herein as sufficiently identical.

In a preferred embodiment, an HNMDA-1 polypeptide includes at least one or more of the following domains: a transmembrane domain, a signal peptide, a ligand-gated ion channel family domain, and/or a glutamate-gated ion channel family domain, and has an amino acid sequence at least about 50%, 55%, 60%, 65%, 70%, 75%, 80%, 85%, 85%, 90%, 95%, 96%, 97%, 98%, 99% or more homologous or identical to the amino acid sequence of SEQ ID NO:38, or the amino acid sequence encoded by the DNA insert of the plasmid deposited with ATCC as Accession Number ______. In yet another preferred embodiment, an HNMDA-1 polypeptide includes at least one or more of the following domains: a transmembrane domain, a signal peptide, a ligand-gated ion channel family domain, and/or a glutamate-gated ion channel family domain, and is encoded by a nucleic acid molecule having a nucleotide sequence which hybridizes under stringent hybridization conditions to a complement of a nucleic acid molecule comprising the nucleotide sequence of SEQ ID NO:37 or 39. In another preferred embodiment, an HNMDA-1 polypeptide includes at least one or more of the following domains: a transmembrane domain, a signal peptide, a ligand-gated ion channel family domain, and/or a glutamate-gated ion channel family domain, and has an HNMDA-1 activity.

As used interchangeably herein, an “HNMDA-1 activity”, “biological activity of HNMDA-1” or “functional activity of HNMDA-1,” refers to an activity exerted by an HNMDA-1 polypeptide or nucleic acid molecule on an HNMDA-1 responsive cell or tissue, or on an HNMDA-1 polypeptide substrate, as determined in vivo, or in vitro, according to standard techniques. In one embodiment, an HNMDA-1 activity is a direct activity, such as an association with an HNMDA-1-target molecule. As used herein, a “substrate,” “target molecule,” or “binding partner” is a molecule with which an HNMDA-1 polypeptide binds or interacts in nature, such that HNMDA-1-mediated function is achieved. An HNMDA-1 target molecule can be a non-HNMDA-1 molecule or an HNMDA-1 polypeptide or polypeptide of the present invention. In an exemplary embodiment, an HNMDA-1 target molecule is an HNMDA-1 ligand, e.g., a glutamate-gated ion channel ligand such as L-glutamate or glycine. Alternatively, an HNMDA-1 activity is an indirect activity, such as a cellular signaling activity mediated by interaction of the HNMDA-1 polypeptide with an HNMDA-1 ligand. The biological activities of HNMDA-1 are described herein. For example, the HNMDA-1 polypeptides of the present invention can have one or more of the following activities: (1) modulate Ca²⁺ transport across a cell membrane, (2) modulate intracellular Ca²⁺ concentration, (3) bind a ligand, e.g., L-glutamate, and/or glycine, (4) influence long term synapse potentiation, (5) modulate synapse formation, e.g., synapse formation related to memory or learning, and/or (6) modulate synapse formation related to the formation of neural networks during development.

The nucleotide sequence of the isolated HNMDA-1 cDNA and the predicted amino acid sequence of the HNMDA-1 polypeptide are shown in FIG. 32 and in SEQ ID NOs:37 and 38, respectively. A plasmid containing the nucleotide sequence encoding HNMDA-1 was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The HNMDA-1 gene, which is approximately 4197 nucleotides in length, encodes a polypeptide which is approximately 1115 amino acid residues in length.

Various aspects of the invention are described in further detail in the following subsections:

I. Isolated Nucleic Acid Molecules

One aspect of the invention pertains to isolated nucleic acid molecules that encode HNMDA-1 polypeptides or biologically active portions thereof, as well as nucleic acid fragments sufficient for use as hybridization probes to identify HNMDA-1-encoding nucleic acid molecules (e.g., HNMDA-1 mRNA) and fragments for use as PCR primers for the amplification or mutation of HNMDA-1 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated HNMDA-1 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:37 or 39, or the nucleotide sequence of the DNA insert of the plasmid deposited with ATCC as Accession Number ______, or a portion thereof, can be isolated using standard molecular biology techniques and the sequence information provided herein. Using all or a portion of the nucleic acid sequence of SEQ ID NO:37 or 39, or the nucleotide sequence of the DNA insert of the plasmid deposited with ATCC as Accession Number ______, as a hybridization probe, HNMDA-1 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:37 or 39, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:37 or 39, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to HNMDA-1 nucleotide sequences can be prepared by standard synthetic techniques, e.g., using an automated DNA synthesizer.

In one embodiment, an isolated nucleic acid molecule of the invention comprises the nucleotide sequence shown in SEQ ID NO:37. The sequence of SEQ ID NO:37 corresponds to the HNMDA-1 cDNA. This cDNA comprises sequences encoding the HNMDA-1 polypeptide (i.e., “the coding region”, from nucleotides 1-3348) as well as 3′ untranslated sequences (nucleotides 3349-4197). Alternatively, the nucleic acid molecule can comprise only the coding region of SEQ ID NO:37 (e.g., nucleotides 1-3348, corresponding to SEQ ID NO:39). Accordingly, in another embodiment, the isolated nucleic acid molecule comprises SEQ ID NO:39 and nucleotides 3349-4197 of SEQ ID NO:37. In yet another embodiment, the nucleic acid molecule consists of the nucleotide sequence set forth as SEQ ID NO:37 or 39.

In still another embodiment, an isolated nucleic acid molecule of the invention comprises a nucleic acid molecule which is a complement of the nucleotide sequence shown in SEQ ID NO:37 or 39, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:37 or 39, or the nucleotide sequence of the DNA insert of the plasmid deposited with ATCC as Accession Number ______, is one which is sufficiently complementary to the nucleotide sequence shown in SEQ ID NO:37 or 39, or the nucleotide sequence of the DNA insert of the plasmid deposited with ATCC as Accession Number ______, such that it can hybridize to the nucleotide sequence shown in SEQ ID NO:37 or 39, or the nucleotide sequence of the DNA insert of the plasmid deposited with ATCC as Accession Number ______, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 96%, 97%, 98%, 99% or more identical to the nucleotide sequence shown in SEQ ID NO:37 or 39 (e.g., to the entire length of the nucleotide sequence), or to the nucleotide sequence (e.g., the entire length of the nucleotide sequence) of the DNA insert of the plasmid deposited with ATCC as Accession Number ______, or a portion of any of these nucleotide sequences. In one embodiment, a nucleic acid molecule of the present invention comprises a nucleotide sequence which is at least (or no greater than) 50-100, 100-250, 250-500, 500-750, 750-1000, 1000-1250, 1250-1500, 1500-1750, 1750-1900, 1900-2150, 2150-2400, 2400-2650, 2650-2900, 2900-3150, 3150-3400, 3400-3650, 3650-3900, 3900-4150 or more nucleotides in length and hybridizes under stringent hybridization conditions to a complement of a nucleic acid molecule of SEQ ID NO:37 or 39, or the nucleotide sequence of the DNA insert of the plasmid deposited with ATCC as Accession Number ______.

Moreover, the nucleic acid molecule of the invention can comprise only a portion of the nucleic acid sequence of SEQ ID NO:37 or 39, or the nucleotide sequence of the DNA insert of the plasmid deposited with ATCC as Accession Number ______, for example, a fragment which can be used as a probe or primer or a fragment encoding a portion of an HNMDA-1 polypeptide, e.g., a biologically active portion of an HNMDA-1 polypeptide. The nucleotide sequence determined from the cloning of the HNMDA-1 gene allows for the generation of probes and primers designed for use in identifying and/or cloning other HNMDA-1 family members, as well as HNMDA-1 homologues from other species. The probe/primer typically comprises substantially purified oligonucleotide. The probe/primer (e.g., oligonucleotide) typically comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95, or 100 or more consecutive nucleotides of a sense sequence of SEQ ID NO:37 or 39, or the nucleotide sequence of the DNA insert of the plasmid deposited with ATCC as Accession Number ______, of an anti-sense sequence of SEQ ID NO:37 or 39, or the nucleotide sequence of the DNA insert of the plasmid deposited with ATCC as Accession Number ______, or of a naturally occurring allelic variant or mutant of SEQ ID NO:37 or 39, or the nucleotide sequence of the DNA insert of the plasmid deposited with ATCC as Accession Number ______.

Exemplary probes or primers are at least 12, 15, 20, 25, 30, 35, 40, 45, 50, 55, 60, 65, 70, 75 or more nucleotides in length and/or comprise consecutive nucleotides of an isolated nucleic acid molecule described herein. Probes based on the HNMDA-1 nucleotide sequences can be used to detect (e.g., specifically detect) transcripts or genomic sequences encoding the same or homologous polypeptides. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of an HNMDA-1 sequence, e.g., a domain, region, site or other sequence described herein. The primers should be at least 5, 10, 20, 30, 40, 50, 60, 70, 80, 90, 100 or more nucleotides in length. Such probes can be used as a part of a diagnostic test kit for identifying cells or tissue which misexpress an HNMDA-1 polypeptide, such as by measuring a level of an HNMDA-1-encoding nucleic acid in a sample of cells from a subject e.g., detecting HNMDA-1 mRNA levels or determining whether a genomic HNMDA-1 gene has been mutated or deleted.

A nucleic acid fragment encoding a “biologically active portion of an HNMDA-1 polypeptide” can be prepared by isolating a portion of the nucleotide sequence of SEQ ID NO:37 or 39, or the nucleotide sequence of the DNA insert of the plasmid deposited with ATCC as Accession Number ______, which encodes a polypeptide having an HNMDA-1 biological activity (the biological activities of the HNMDA-1 polypeptides are described herein), expressing the encoded portion of the HNMDA-1 polypeptide (e.g., by recombinant expression in vitro) and assessing the activity of the encoded portion of the HNMDA-1 polypeptide. In an exemplary embodiment, the nucleic acid molecule is at least 50-100, 100-250, 250-500, 500-750, 750-1000, 1000-1250, 1250-1500, 1500-1750, 1750-1900, 1900-2150, 2150-2400, 2400-2650, 2650-2900, 2900-3150, 3150-3400, 3400-3650, 3650-3900, 3900-4150 or more nucleotides in length and encodes a polypeptide having an HNMDA-1 activity (as described herein).

The invention further encompasses nucleic acid molecules that differ from the nucleotide sequence shown in SEQ ID NO:37 or 39, or the nucleotide sequence of the DNA insert of the plasmid deposited with ATCC as Accession Number ______. Such differences can be due to due to degeneracy of the genetic code, thus resulting in a nucleic acid which encodes the same HNMDA-1 polypeptides as those encoded by the nucleotide sequence shown in SEQ ID NO:37 or 39, or the nucleotide sequence of the DNA insert of the plasmid deposited with ATCC as Accession Number ______. In another embodiment, an isolated nucleic acid molecule of the invention has a nucleotide sequence encoding a polypeptide having an amino acid sequence which differs by at least 1, but no greater than 5, 10, 20, 50 or 100 amino acid residues from the amino acid sequence shown in SEQ ID NO:38, or the amino acid sequence encoded by the DNA insert of the plasmid deposited with the ATCC as Accession Number ______. In yet another embodiment, the nucleic acid molecule encodes the amino acid sequence of HNMDA-1. If an alignment is needed for this comparison, the sequences should be aligned for maximum homology.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

Allelic variants result, for example, from DNA sequence polymorphisms within a population (e.g., the human population) that lead to changes in the amino acid sequences of the HNMDA-1 polypeptides. Such genetic polymorphism in the HNMDA-1 genes may exist among individuals within a population due to natural allelic variation. As used herein, the terms “gene” and “recombinant gene” refer to nucleic acid molecules which include an open reading frame encoding an HNMDA-1 polypeptide, preferably a mammalian HNMDA-1 polypeptide, and can further include non-coding regulatory sequences, and introns.

Accordingly, in one embodiment, the invention features isolated nucleic acid molecules which encode a naturally occurring allelic variant of a polypeptide comprising the amino acid sequence of SEQ ID NO:38, or an amino acid sequence encoded by the DNA insert of the plasmid deposited with ATCC as Accession Number ______, wherein the nucleic acid molecule hybridizes to a complement of a nucleic acid molecule comprising SEQ ID NO:37 or 39, for example, under stringent hybridization conditions.

Allelic variants of HNMDA-1 include both functional and non-functional HNMDA-1 polypeptides. Functional allelic variants are naturally occurring amino acid sequence variants of the HNMDA-1 polypeptide that have an HNMDA-1 activity, e.g., maintain the ability to bind an HNMDA-1 ligand or substrate (e.g., L-glutamate and/or glycine) and/or modulate Ca²⁺ transport. Functional allelic variants will typically contain only conservative substitution of one or more amino acids of SEQ ID NO:38, or substitution, deletion or insertion of non-critical residues in non-critical regions of the polypeptide.

Non-functional allelic variants are naturally occurring amino acid sequence variants of the HNMDA-1 polypeptide that do not have an HNMDA-1 activity, e.g., they do not have the ability to transport Ca²⁺ into and out of cells or to bind L-glutamate and/or glycine. Non-functional allelic variants will typically contain a non-conservative substitution, a deletion, or insertion or premature truncation of the amino acid sequence of SEQ ID NO:38, or a substitution, insertion or deletion in critical residues or critical regions.

The present invention further provides non-human orthologues of the HNMDA-1 polypeptide. Orthologues of HNMDA-1 polypeptides are polypeptides that are isolated from non-human organisms and possess the same HNMDA-1 activity, e.g., ligand binding and/or Ca²⁺ transport, as the HNMDA-1 polypeptide. Orthologues of the HNMDA-1 polypeptide can readily be identified as comprising an amino acid sequence that is substantially identical to SEQ ID NO:38.

Moreover, nucleic acid molecules encoding other HNMDA-1 family members and, thus, which have a nucleotide sequence which differs from the HNMDA-1 sequences of SEQ ID NO:37 or 39, or the nucleotide sequence of the DNA insert of the plasmid deposited with ATCC as Accession Number ______, are intended to be within the scope of the invention. For example, another HNMDA-1 cDNA can be identified based on the nucleotide sequence of HNMDA-1. Moreover, nucleic acid molecules encoding HNMDA-1 polypeptides from different species, and which, thus, have a nucleotide sequence which differs from the HNMDA-1 sequences of SEQ ID NO:37 or 39, or the nucleotide sequence of the DNA insert of the plasmid deposited with ATCC as Accession Number ______ are intended to be within the scope of the invention. For example, a mouse HNMDA-1 cDNA can be identified based on the nucleotide sequence of a HNMDA-1.

Nucleic acid molecules corresponding to natural allelic variants and homologues of the HNMDA-1 cDNAs of the invention can be isolated based on their homology to the HNMDA-1 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the HNMDA-1 cDNAs of the invention can further be isolated by mapping to the same chromosome or locus as the HNMDA-1 gene.

Orthologues, homologues and allelic variants can be identified using methods known in the art (e.g., by hybridization to an isolated nucleic acid molecule of the present invention, for example, under stringent hybridization conditions). In one embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:37 or 39, or the nucleotide sequence of the DNA insert of the plasmid deposited with ATCC as Accession Number ______. In other embodiment, the nucleic acid is at least 100, 150, 200, 250, 300, 350, 400, 450, 500, 550, 600, 650, 700, 750, 800, 850, 900, 950, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

Preferably, an isolated nucleic acid molecule of the invention that hybridizes under stringent conditions to the sequence of SEQ ID NO:37 or 39 and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural polypeptide).

In addition to naturally-occurring allelic variants of the HNMDA-1 sequences that may exist in the population, the skilled artisan will further appreciate that changes can be introduced by mutation into the nucleotide sequences of SEQ ID NO:37 or 39, or the nucleotide sequence of the DNA insert of the plasmid deposited with ATCC as Accession Number ______, thereby leading to changes in the amino acid sequence of the encoded HNMDA-1 polypeptides, without altering the functional ability of the HNMDA-1 polypeptides. For example, nucleotide substitutions leading to amino acid substitutions at “non-essential” amino acid residues can be made in the sequence of SEQ ID NO:37 or 39, or the nucleotide sequence of the DNA insert of the plasmid deposited with ATCC as Accession Number ______. A “non-essential” amino acid residue is a residue that can be altered from the wild-type sequence of HNMDA-1 (e.g., the sequence of SEQ ID NO:38) without altering the biological activity, whereas an “essential” amino acid residue is required for biological activity. For example, amino acid residues that are conserved among the HNMDA-1 polypeptides of the present invention, e.g., those present in a transmembrane domain, a signal peptide, a ligand-gated ion channel family domain, and/or a glutamate-gated ion channel family domain, are predicted to be particularly unamenable to alteration. Furthermore, additional amino acid residues that are conserved between the HNMDA-1 polypeptides of the present invention and other members of the HNMDA-1 family are not likely to be amenable to alteration.

Accordingly, another aspect of the invention pertains to nucleic acid molecules encoding HNMDA-1 polypeptides that contain changes in amino acid residues that are not essential for activity. Such HNMDA-1 polypeptides differ in amino acid sequence from SEQ ID NO:38, yet retain biological activity. In one embodiment, the isolated nucleic acid molecule comprises a nucleotide sequence encoding a polypeptide, wherein the polypeptide comprises an amino acid sequence at least about 50%, 55%, 60%, 65%, 70%, 75%, 80%, 85%, 90%, 95%, 96%, 97%, 98%, 99% or more identical to SEQ ID NO:38 (e.g., to the entire length of SEQ ID NO:38).

An isolated nucleic acid molecule encoding an HNMDA-1 polypeptide identical to the polypeptide of SEQ ID NO:38, can be created by introducing one or more nucleotide substitutions, additions or deletions into the nucleotide sequence of SEQ ID NO:37 or 39, or the nucleotide sequence of the DNA insert of the plasmid deposited with ATCC as Accession Number _______, such that one or more amino acid substitutions, additions or deletions are introduced into the encoded polypeptide. Mutations can be introduced into SEQ ID NO:37 or 39, or the nucleotide sequence of the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an HNMDA-1 polypeptide is preferably replaced with another amino acid residue from the same side chain family. Alternatively, in another embodiment, mutations can be introduced randomly along all or part of an HNMDA-1 coding sequence, such as by saturation mutagenesis, and the resultant mutants can be screened for HNMDA-1 biological activity to identify mutants that retain activity. Following mutagenesis of SEQ ID NO:37 or 39, or the nucleotide sequence of the DNA insert of the plasmid deposited with ATCC as Accession Number ______, the encoded polypeptide can be expressed recombinantly and the activity of the polypeptide can be determined.

In a preferred embodiment, a mutant HNMDA-1 polypeptide can be assayed for the ability to (1) modulate Ca²⁺ transport across a cell membrane, (2) modulate intracellular Ca²⁺ concentration, (3) bind a ligand, e.g., L-glutamate, and/or glycine, (4) influence long term synapse potentiation, (5) modulate synapse formation, e.g., synapse formation related to memory or learning, and/or (6) modulate synapse formation related to the formation of neural networks during development.

In addition to the nucleic acid molecules encoding HNMDA-1 polypeptides described above, another aspect of the invention pertains to isolated nucleic acid molecules which are antisense thereto. In an exemplary embodiment, the invention provides an isolated nucleic acid molecule which is antisense to an HNMDA-1 nucleic acid molecule (e.g., is antisense to the coding strand of an HNMDA-1 nucleic acid molecule). An “antisense” nucleic acid comprises a nucleotide sequence which is complementary to a “sense” nucleic acid encoding a polypeptide, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire HNMDA-1 coding strand, or to only a portion thereof. In one embodiment, an antisense nucleic acid molecule is antisense to a “coding region” of the coding strand of a nucleotide sequence encoding HNMDA-1. The term “coding region” refers to the region of the nucleotide sequence comprising codons which are translated into amino acid residues (e.g., the coding region of HNMDA-1 corresponds to SEQ ID NO:39). In another embodiment, the antisense nucleic acid molecule is antisense to a “noncoding region” of the coding strand of a nucleotide sequence encoding HNMDA-1. The term “noncoding region” refers to 5′ and 3′ sequences which flank the coding region that are not translated into amino acids (i.e., also referred to as 5′ and 3′ untranslated regions).

Given the coding strand sequences encoding HNMDA-1 disclosed herein (e.g., SEQ ID NO:39), antisense nucleic acids of the invention can be designed according to the rules of Watson and Crick base pairing. The antisense nucleic acid molecule can be complementary to the entire coding region of HNMDA-1 mRNA, but more preferably is an oligonucleotide which is antisense to only a portion of the coding or noncoding region of HNMDA-1 mRNA. For example, the antisense oligonucleotide can be complementary to the region surrounding the translation start site of HNMDA-1 mRNA (e.g., between the −10 and +10 regions of the start site of a gene nucleotide sequence). An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an HNMDA-1 polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave HNMDA-1 mRNA transcripts to thereby inhibit translation of HNMDA-1 mRNA. A ribozyme having specificity for an HNMDA-1-encoding nucleic acid can be designed based upon the nucleotide sequence of an HNMDA-1 cDNA disclosed herein (i.e., SEQ ID NO:37 or 39, or the nucleotide sequence of the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in an HNMDA-1-encoding mRNA. See, e.g., Cech et al. U.S. Pat. No. 4,987,071; and Cech et al. U.S. Pat. No. 5,116,742. Alternatively, HNMDA-1 mRNA can be used to select a catalytic RNA having a specific ribonuclease activity from a pool of RNA molecules. See, e.g., Bartel, D. and Szostak, J. W. (1993) Science 261:1411-1418.

Alternatively, HNMDA-1 gene expression can be inhibited by targeting nucleotide sequences complementary to the regulatory region of the HNMDA-1 (e.g., the HNMDA-1 promoter and/or enhancers) to form triple helical structures that prevent transcription of the HNMDA-1 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the HNMDA-1 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of HNMDA-1 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of HNMDA-1 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of HNMDA-1 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of HNMDA-1 nucleic acid molecules can be generated which may combine the advantageous properties of PNA and DNA. Such chimeras allow DNA recognition enzymes, (e.g., RNase H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous HNMDA-1 gene within a cell line or microorganism may be modified by inserting a heterologous DNA regulatory element into the genome of a stable cell line or cloned microorganism such that the inserted regulatory element is operatively linked with the endogenous HNMDA-1 gene. For example, an endogenous HNMDA-1 gene which is normally “transcriptionally silent”, i.e., an HNMDA-1 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous HNMDA-1 gene may be activated by insertion of a promiscuous regulatory element that works across cell types.

A heterologous regulatory element may be inserted into a stable cell line or cloned microorganism, such that it is operatively linked with an endogenous HNMDA-1 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated HNMDA-1 Polypeptides and Anti-HNMDA-1 Antibodies

One aspect of the invention pertains to isolated HNMDA-1 or recombinant polypeptides and polypeptides, and biologically active portions thereof, as well as polypeptide fragments suitable for use as immunogens to raise anti-HNMDA-1 antibodies. In one embodiment, native HNMDA-1 polypeptides can be isolated from cells or tissue sources by an appropriate purification scheme using standard protein purification techniques. In another embodiment, HNMDA-1 polypeptides are produced by recombinant DNA techniques. Alternative to recombinant expression, an HNMDA-1 polypeptide or polypeptide can be synthesized chemically using standard peptide synthesis techniques.

An “isolated” or “purified” polypeptide or biologically active portion thereof is substantially free of cellular material or other contaminating proteins from the cell or tissue source from which the HNMDA-1 polypeptide is derived, or substantially free from chemical precursors or other chemicals when chemically synthesized. The language “substantially free of cellular material” includes preparations of HNMDA-1 polypeptide in which the polypeptide is separated from cellular components of the cells from which it is isolated or recombinantly produced. In one embodiment, the language “substantially free of cellular material” includes preparations of HNMDA-1 polypeptide having less than about 30% (by dry weight) of non-HNMDA-1 polypeptide (also referred to herein as a “contaminating protein”), more preferably less than about 20% of non-HNMDA-1 polypeptide, still more preferably less than about 10% of non-HNMDA-1 polypeptide, and most preferably less than about 5% non-HNMDA-1 polypeptide. When the HNMDA-1 polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of HNMDA-1 polypeptide in which the polypeptide is separated from chemical precursors or other chemicals which are involved in the synthesis of the polypeptide. In one embodiment, the language “substantially free of chemical precursors or other chemicals” includes preparations of HNMDA-1 polypeptide having less than about 30% (by dry weight) of chemical precursors or non-HNMDA-1 chemicals, more preferably less than about 20% chemical precursors or non-HNMDA-1 chemicals, still more preferably less than about 10% chemical precursors or non-HNMDA-1 chemicals, and most preferably less than about 5% chemical precursors or non-HNMDA-1 chemicals.

As used herein, a “biologically active portion” of an HNMDA-1 polypeptide includes a fragment of an HNMDA-1 polypeptide which participates in an interaction between an HNMDA-1 molecule and a non-HNMDA-1 molecule. Biologically active portions of an HNMDA-1 polypeptide include peptides comprising amino acid sequences sufficiently identical to or derived from the amino acid sequence of the HNMDA-1 polypeptide, e.g., the amino acid sequence shown in SEQ ID NO:38, which include less amino acids than the full length HNMDA-1 polypeptides, and exhibit at least one activity of an HNMDA-1 polypeptide. Typically, biologically active portions comprise a domain or motif with at least one activity of the HNMDA-1 polypeptide, e.g., the ability to bind L-glutamate or glycine or the ability to modulate Ca²⁺ transport. A biologically active portion of an HNMDA-1 polypeptide can be a polypeptide which is, for example, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100 or more amino acids in length. Biologically active portions of an HNMDA-1 polypeptide can be used as targets for developing agents which modulate an HNMDA-1 mediated activity, e.g., a long term synapse potentiation.

In one embodiment, a biologically active portion of an HNMDA-1 polypeptide comprises at least one glutamate-gated ion channel family. It is to be understood that a preferred biologically active portion of an HNMDA-1 polypeptide of the present invention comprises at least one or more of the following domains: a transmembrane domain, a signal peptide, a ligand-gated ion channel family domain and/or a glutamate-gated ion channel family domain. Moreover, other biologically active portions, in which other regions of the polypeptide are deleted, can be prepared by recombinant techniques and evaluated for one or more of the functional activities of a native HNMDA-1 polypeptide.

Another aspect of the invention features fragments of the polypeptide having the amino acid sequence of SEQ ID NO:38, for example, for use as immunogens. In one embodiment, a fragment comprises at least 5 amino acids (e.g., contiguous or consecutive amino acids) of the amino acid sequence of SEQ ID NO:38, or an amino acid sequence encoded by the DNA insert of the plasmid deposited with the ATCC as Accession Number ______. In another embodiment, a fragment comprises at least 10, 15, 20, 25, 30, 35, 40, 45, 50 or more amino acids (e.g., contiguous or consecutive amino acids) of the amino acid sequence of SEQ ID NO:38, or an amino acid sequence encoded by the DNA insert of the plasmid deposited with the ATCC as Accession Number ______.

In a preferred embodiment, an HNMDA-1 polypeptide has an amino acid sequence shown in SEQ ID NO:38. In other embodiments, the HNMDA-1 polypeptide is substantially identical to SEQ ID NO:38, and retains the functional activity of the polypeptide of SEQ ID NO:38, yet differs in amino acid sequence due to natural allelic variation or mutagenesis, as described in detail in subsection I above. In another embodiment, the HNMDA-1 polypeptide is a polypeptide which comprises an amino acid sequence at least about 50%, 55%, 60%, 65%, 70%, 75%, 80%, 85%, 90%, 95%, 96%, 97%, 98%, 99% or more identical to SEQ ID NO:38.

In another embodiment, the invention features an HNMDA-1 polypeptide which is encoded by a nucleic acid molecule consisting of a nucleotide sequence at least about 50%, 55%, 60%, 65%, 70%, 75%, 80%, 85%, 90%, 95%, 96%, 97%, 98%, 99% or more identical to a nucleotide sequence of SEQ ID NO:37 or 39, or a complement thereof. This invention further features an HNMDA-1 polypeptide which is encoded by a nucleic acid molecule consisting of a nucleotide sequence which hybridizes under stringent hybridization conditions to a complement of a nucleic acid molecule comprising the nucleotide sequence of SEQ ID NO:37 or 39, or a complement thereof.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the HNMDA-1 amino acid sequence of SEQ ID NO:38 having 1115 amino acid residues, at least 334, preferably at least 446, more preferably at least 557, more preferably at least 669, even more preferably at least 780, and even more preferably at least 892 or 1003 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of E. Meyers and W. Miller (Comput. Appl. Biosci., 4:11-17 (1988)) which has been incorporated into the ALIGN program (version 2.0 or version 2.0U), using a PAM120 weight residue table, a gap length penalty of 12 and a gap penalty of 4.

The nucleic acid and polypeptide sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to HNMDA-1 nucleic acid molecules of the invention. BLAST protein searches can be performed with the XBLAST program, score=100, wordlength=3, and a Blosum62 matrix to obtain amino acid sequences homologous to HNMDA-1 polypeptide molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides HNMDA-1 chimeric or fusion proteins. As used herein, an HNMDA-1 “chimeric protein” or “fusion protein” comprises an HNMDA-1 polypeptide operatively linked to a non-HNMDA-1 polypeptide. An “HNMDA-1 polypeptide” refers to a polypeptide having an amino acid sequence corresponding to HNMDA-1, whereas a “non-HNMDA-1 polypeptide” refers to a polypeptide having an amino acid sequence corresponding to a polypeptide which is not substantially homologous to the HNMDA-1 polypeptide, e.g., a polypeptide which is different from the HNMDA-1 polypeptide and which is derived from the same or a different organism. Within an HNMDA-1 fusion protein the HNMDA-1 polypeptide can correspond to all or a portion of an HNMDA-1 polypeptide. In a preferred embodiment, an HNMDA-1 fusion protein comprises at least one biologically active portion of an HNMDA-1 polypeptide. In another preferred embodiment, an HNMDA-1 fusion protein comprises at least two biologically active portions of an HNMDA-1 polypeptide. Within the fusion protein, the term “operatively linked” is intended to indicate that the HNMDA-1 polypeptide and the non-HNMDA-1 polypeptide are fused in-frame to each other. The non-HNMDA-1 polypeptide can be fused to the N-terminus or C-terminus of the HNMDA-1 polypeptide.

For example, in one embodiment, the fusion protein is a GST-HNMDA-1 fusion protein in which the HNMDA-1 sequences are fused to the C-terminus of the GST sequences. Such fusion proteins can facilitate the purification of recombinant HNMDA-1.

In another embodiment, the fusion protein is an HNMDA-1 polypeptide containing a heterologous signal sequence at its N-terminus. In certain host cells (e.g., mammalian host cells), expression and/or secretion of HNMDA-1 can be increased through the use of a heterologous signal sequence.

The HNMDA-1 fusion proteins of the invention can be incorporated into pharmaceutical compositions and administered to a subject in vivo. The HNMDA-1 fusion proteins can be used to affect the bioavailability of an HNMDA-1 substrate. Use of HNMDA-1 fusion proteins may be useful therapeutically for the treatment of disorders caused by, for example, (i) aberrant modification or mutation of a gene encoding an HNMDA-1 polypeptide; (ii) mis-regulation of the HNMDA-1 gene; and (iii) aberrant post-translational modification of an HNMDA-1 polypeptide.

Moreover, the HNMDA-1-fusion proteins of the invention can be used as immunogens to produce anti-HNMDA-1 antibodies in a subject, to purify HNMDA-1 ligands and in screening assays to identify molecules which inhibit the interaction of HNMDA-1 with an HNMDA-1 substrate.

Preferably, an HNMDA-1 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An HNMDA-1-encoding nucleic acid can be cloned into such an expression vector such that the fusion moiety is linked in-frame to the HNMDA-1 polypeptide.

The present invention also pertains to variants of the HNMDA-1 polypeptides which function as either HNMDA-1 agonists (mimetics) or as HNMDA-1 antagonists. Variants of the HNMDA-1 polypeptides can be generated by mutagenesis, e.g., discrete point mutation or truncation of an HNMDA-1 polypeptide. An agonist of the HNMDA-1 polypeptides can retain substantially the same, or a subset, of the biological activities of the naturally occurring form of an HNMDA-1 polypeptide. An antagonist of an HNMDA-1 polypeptide can inhibit one or more of the activities of the naturally occurring form of the HNMDA-1 polypeptide by, for example, competitively modulating an HNMDA-1-mediated activity of an HNMDA-1 polypeptide. Thus, specific biological effects can be elicited by treatment with a variant of limited function. In one embodiment, treatment of a subject with a variant having a subset of the biological activities of the naturally occurring form of the polypeptide has fewer side effects in a subject relative to treatment with the naturally occurring form of the HNMDA-1 polypeptide.

In one embodiment, variants of an HNMDA-1 polypeptide which function as either HNMDA-1 agonists (mimetics) or as HNMDA-1 antagonists can be identified by screening combinatorial libraries of mutants, e.g., truncation mutants, of an HNMDA-1 polypeptide for HNMDA-1 polypeptide agonist or antagonist activity. In one embodiment, a variegated library of HNMDA-1 variants is generated by combinatorial mutagenesis at the nucleic acid level and is encoded by a variegated gene library. A variegated library of HNMDA-1 variants can be produced by, for example, enzymatically ligating a mixture of synthetic oligonucleotides into gene sequences such that a degenerate set of potential HNMDA-1 sequences is expressible as individual polypeptides, or alternatively, as a set of larger fusion proteins (e.g., for phage display) containing the set of HNMDA-1 sequences therein. There are a variety of methods which can be used to produce libraries of potential HNMDA-1 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential HNMDA-1 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of an HNMDA-1 polypeptide coding sequence can be used to generate a variegated population of HNMDA-1 fragments for screening and subsequent selection of variants of an HNMDA-1 polypeptide. In one embodiment, a library of coding sequence fragments can be generated by treating a double stranded PCR fragment of an HNMDA-1 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the HNMDA-1 polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of HNMDA-1 polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify HNMDA-1 variants (Arkin and Youvan (1992) Proc. Natl. Acad. Sci. USA 89:7811-7815; Delagrave et al. (1993) Protein Engineering 6(3):327-331).

In one embodiment, cell based assays can be exploited to analyze a variegated HNMDA-1 library. For example, a library of expression vectors can be transfected into a cell line, e.g., a neural cell line, which ordinarily responds to HNMDA-1 in a particular HNMDA-1 substrate-dependent manner. The transfected cells are then contacted with HNMDA-1 and the effect of expression of the mutant on signaling by the HNMDA-1 substrate can be detected, e.g., by monitoring intracellular calcium, IP3, or diacylglycerol concentration, phosphorylation profile of intracellular proteins, or the activity of an HNMDA-1-regulated transcription factor. Plasmid DNA can then be recovered from the cells which score for inhibition, or alternatively, potentiation of signaling by the HNMDA-1 substrate, and the individual clones further characterized.

An isolated HNMDA-1 polypeptide, or a portion or fragment thereof, can be used as an immunogen to generate antibodies that bind HNMDA-1 using standard techniques for polyclonal and monoclonal antibody preparation. A full-length HNMDA-1 polypeptide can be used or, alternatively, the invention provides antigenic peptide fragments of HNMDA-1 for use as immunogens. The antigenic peptide of HNMDA-1 comprises at least 8 amino acid residues of the amino acid sequence shown in SEQ ID NO:38 and encompasses an epitope of HNMDA-1 such that an antibody raised against the peptide forms a specific immune complex with HNMDA-1. Preferably, the antigenic peptide comprises at least 10 amino acid residues, more preferably at least 15 amino acid residues, even more preferably at least 20 amino acid residues, and most preferably at least 30 amino acid residues.

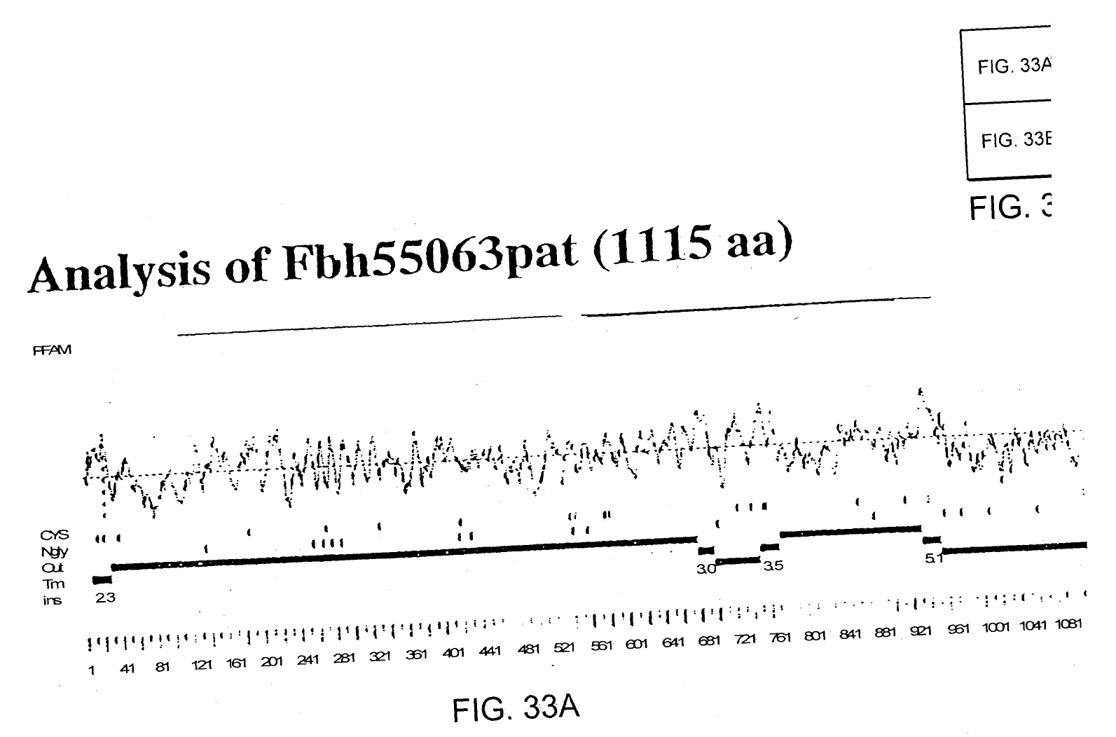

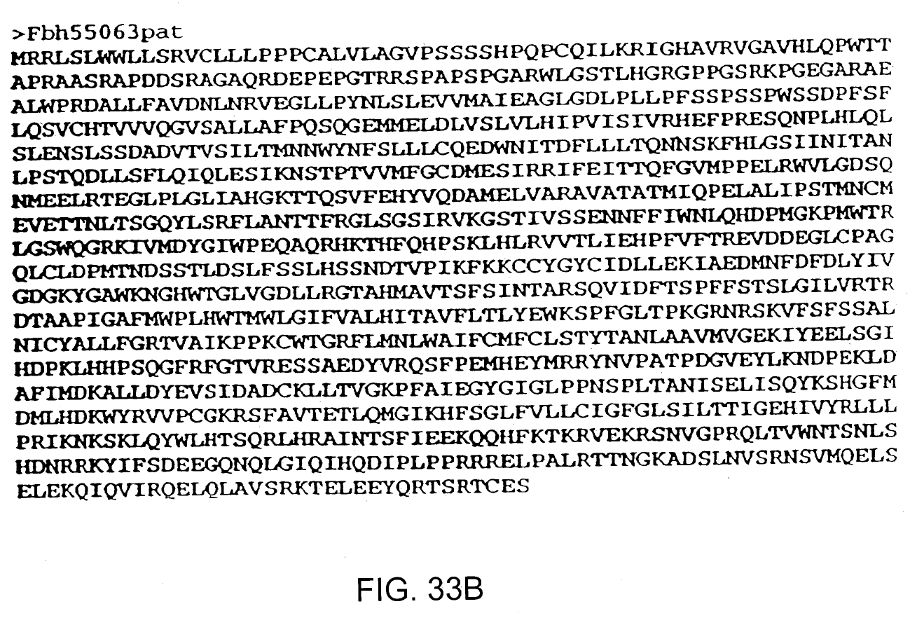

Preferred epitopes encompassed by the antigenic peptide are regions of HNMDA-1 that are located on the surface of the polypeptide, e.g., hydrophilic regions, as well as regions with high antigenicity (see, for example, FIG. 33).

An HNMDA-1 immunogen typically is used to prepare antibodies by immunizing a suitable subject, (e.g., rabbit, goat, mouse or other mammal) with the immunogen. An appropriate immunogenic preparation can contain, for example, recombinantly expressed HNMDA-1 polypeptide or a chemically synthesized HNMDA-1 polypeptide. The preparation can further include an adjuvant, such as Freund's complete or incomplete adjuvant, or similar immunostimulatory agent. Immunization of a suitable subject with an immunogenic HNMDA-1 preparation induces a polyclonal anti-HNMDA-1 antibody response.

Accordingly, another aspect of the invention pertains to anti-HNMDA-1 antibodies. The term “antibody” as used herein refers to immunoglobulin molecules and immunologically active portions of immunoglobulin molecules, i.e., molecules that contain an antigen binding site which specifically binds (immunoreacts with) an antigen, such as HNMDA-1. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind HNMDA-1. The term “monoclonal antibody” or “monoclonal antibody composition”, as used herein, refers to a population of antibody molecules that contain only one species of an antigen binding site capable of immunoreacting with a particular epitope of HNMDA-1. A monoclonal antibody composition thus typically displays a single binding affinity for a particular HNMDA-1 polypeptide with which it immunoreacts.

Polyclonal anti-HNMDA-1 antibodies can be prepared as described above by immunizing a suitable subject with an HNMDA-1 immunogen. The anti-HNMDA-1 antibody titer in the immunized subject can be monitored over time by standard techniques, such as with an enzyme linked immunosorbent assay (ELISA) using immobilized HNMDA-1. If desired, the antibody molecules directed against HNMDA-1 can be isolated from the mammal (e.g., from the blood) and further purified by well known techniques, such as protein A chromatography to obtain the IgG fraction. At an appropriate time after immunization, e.g., when the anti-HNMDA-1 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem .255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an HNMDA-1 immunogen as described above, and the culture supernatants of the resulting hybridoma cells are screened to identify a hybridoma producing a monoclonal antibody that binds HNMDA-1.

Any of the many well known protocols used for fusing lymphocytes and immortalized cell lines can be applied for the purpose of generating an anti-HNMDA-1 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind HNMDA-1, e.g., using a standard ELISA assay.

Alternative to preparing monoclonal antibody-secreting hybridomas, a monoclonal anti-HNMDA-1 antibody can be identified and isolated by screening a recombinant combinatorial immunoglobulin library (e.g., an antibody phage display library) with HNMDA-1 to thereby isolate immunoglobulin library members that bind HNMDA-1. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-HNMDA-1 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-HNMDA-1 antibody (e.g., monoclonal antibody) can be used to isolate HNMDA-1 by standard techniques, such as affinity chromatography or immunoprecipitation. An anti-HNMDA-1 antibody can facilitate the purification of natural HNMDA-1 from cells and of recombinantly produced HNMDA-1 expressed in host cells. Moreover, an anti-HNMDA-1 antibody can be used to detect HNMDA-1 polypeptide (e.g., in a cellular lysate or cell supernatant) in order to evaluate the abundance and pattern of expression of the HNMDA-1 polypeptide. Anti-HNMDA-1 antibodies can be used diagnostically to monitor polypeptide levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, for example recombinant expression vectors, containing a nucleic acid containing an HNMDA-1 nucleic acid molecule or vectors containing a nucleic acid molecule which encodes an HNMDA-1 polypeptide (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, and the like. The expression vectors of the invention can be introduced into host cells to thereby produce proteins or peptides, including fusion proteins or peptides, encoded by nucleic acids as described herein (e.g., HNMDA-1 polypeptides, mutant forms of HNMDA-1 polypeptides, fusion proteins, and the like).

Accordingly, an exemplary embodiment provides a method for producing a polypeptide, preferably an HNMDA-1 polypeptide, by culturing in a suitable medium a host cell of the invention (e.g., a mammalian host cell such as a non-human mammalian cell) containing a recombinant expression vector, such that the polypeptide is produced.

The recombinant expression vectors of the invention can be designed for expression of HNMDA-1 polypeptides in prokaryotic or eukaryotic cells. For example, HNMDA-1 polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in HNMDA-1 activity assays, (e.g., direct assays or competitive assays described in detail below), or to generate antibodies specific for HNMDA-1 polypeptides, for example. In a preferred embodiment, an HNMDA-1 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the HNMDA-1 expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corporation, San Diego, Calif.).

Alternatively, HNMDA-1 polypeptides can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to HNMDA-1 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which an HNMDA-1 nucleic acid molecule of the invention is introduced, e.g., an HNMDA-1 nucleic acid molecule within a vector (e.g., a recombinant expression vector) or an HNMDA-1 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an HNMDA-1 polypeptide can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an HNMDA-1 polypeptide or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an HNMDA-1 polypeptide. Accordingly, the invention further provides methods for producing an HNMDA-1 polypeptide using the host cells of the invention. In one embodiment, the method comprises culturing the host cell of the invention (into which a recombinant expression vector encoding an HNMDA-1 polypeptide has been introduced) in a suitable medium such that an HNMDA-1 polypeptide is produced. In another embodiment, the method further comprises isolating an HNMDA-1 polypeptide from the medium or the host cell.

The host cells of the invention can also be used to produce non-human transgenic animals. For example, in one embodiment, a host cell of the invention is a fertilized oocyte or an embryonic stem cell into which HNMDA-1-coding sequences have been introduced. Such host cells can then be used to create non-human transgenic animals in which exogenous HNMDA-1 sequences have been introduced into their genome or homologous recombinant animals in which endogenous HNMDA-1 sequences have been altered. Such animals are useful for studying the function and/or activity of an HNMDA-1 and for identifying and/or evaluating modulators of HNMDA-1 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous HNMDA-1 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an HNMDA-1-encoding nucleic acid into the male pronuclei of a fertilized oocyte, e.g., by microinjection, retroviral infection, and allowing the oocyte to develop in a pseudopregnant female foster animal. The HNMDA-1 cDNA sequence of SEQ ID NO:37 can be introduced as a transgene into the genome of a non-human animal. Alternatively, a nonhuman homologue of a HNMDA-1 gene, such as a mouse or rat HNMDA-1 gene, can be used as a transgene. Alternatively, an HNMDA-1 gene homologue, such as another HNMDA-1 family member, can be isolated based on hybridization to the HNMDA-1 cDNA sequences of SEQ ID NO:37 or 39, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to an HNMDA-1 transgene to direct expression of an HNMDA-1 polypeptide to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of an HNMDA-1 transgene in its genome and/or expression of HNMDA-1 mRNA in tissues or cells of the animals. A transgenic founder animal can then be used to breed additional animals carrying the transgene. Moreover, transgenic animals carrying a transgene encoding an HNMDA-1 polypeptide can further be bred to other transgenic animals carrying other transgenes.

To create a homologous recombinant animal, a vector is prepared which contains at least a portion of an HNMDA-1 gene into which a deletion, addition or substitution has been introduced to thereby alter, e.g, functionally disrupt, the HNMDA-1 gene. The HNMDA-1 gene can be a human gene (e.g., the cDNA of SEQ ID NO:39), but more preferably, is a non-human homologue of a HNMDA-1 gene (e.g., a cDNA isolated by stringent hybridization with the nucleotide sequence of SEQ ID NO:37). For example, a mouse HNMDA-1 gene can be used to construct a homologous recombination nucleic acid molecule, e.g., a vector, suitable for altering an endogenous HNMDA-1 gene in the mouse genome. In a preferred embodiment, the homologous recombination nucleic acid molecule is designed such that, upon homologous recombination, the endogenous HNMDA-1 gene is functionally disrupted (i.e., no longer encodes a functional protein; also referred to as a “knock out” vector). Alternatively, the homologous recombination nucleic acid molecule can be designed such that, upon homologous recombination, the endogenous HNMDA-1 gene is mutated or otherwise altered but still encodes functional polypeptide (e.g., the upstream regulatory region can be altered to thereby alter the expression of the endogenous HNMDA-1 polypeptide). In the homologous recombination nucleic acid molecule, the altered portion of the HNMDA-1 gene is flanked at its 5′ and 3′ ends by additional nucleic acid sequence of the HNMDA-1 gene to allow for homologous recombination to occur between the exogenous HNMDA-1 gene carried by the homologous recombination nucleic acid molecule and an endogenous HNMDA-1 gene in a cell, e.g., an embryonic stem cell. The additional flanking HNMDA-1 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced HNMDA-1 gene has homologously recombined with the endogenous HNMDA-1 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The HNMDA-1 nucleic acid molecules, fragments of HNMDA-1 polypeptides, and anti-HNMDA-1 antibodies (also referred to herein as “active compounds”) of the invention can be incorporated into pharmaceutical compositions suitable for administration. Such compositions typically comprise the nucleic acid molecule, polypeptide, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of an HNMDA-1 polypeptide or an anti-HNMDA-1 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a polypeptide or antibody can include a single treatment or, preferably, can include a series of treatments.

In a preferred example, a subject is treated with antibody or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, an HNMDA-1 polypeptide of the invention has one or more of the following activities: (1) modulateCa²⁺ transport across a cell membrane, (2) modulate intracellular Ca²⁺ concentration, (3) bind a ligand, e.g., L-glutamate, and/or glycine, (4) influence long term synapse potentiation, (5) modulate synapse formation, e.g., synapse formation related to memory or learning, and/or (6) modulate synapse formation related to the formation of neural networks during development.

The isolated nucleic acid molecules of the invention can be used, for example, to express HNMDA-1 polypeptides (e.g., via a recombinant expression vector in a host cell in gene therapy applications), to detect HNMDA-1 mRNA (e.g., in a biological sample) or a genetic alteration in an HNMDA-1 gene, and to modulate HNMDA-1 activity, as described further below. The HNMDA-1 polypeptides can be used to treat disorders characterized by insufficient or excessive production of an HNMDA-1 substrate or production of HNMDA-1 inhibitors. In addition, the HNMDA-1 polypeptides can be used to screen for naturally occurring HNMDA-1 substrates, to screen for drugs or compounds which modulate HNMDA-1 activity, as well as to treat disorders characterized by insufficient or excessive production of HNMDA-1 polypeptide or production of HNMDA-1 polypeptide forms which have decreased, aberrant or unwanted activity compared to HNMDA-1 wild type polypeptide (e.g., glutamate-gated ion channel disorders). Moreover, the anti-HNMDA-1 antibodies of the invention can be used to detect and isolate HNMDA-1 polypeptides, to regulate the bioavailability of HNMDA-1 polypeptides, and modulate HNMDA-1 activity.

A. Screening Assays

The invention provides a method (also referred to herein as a “screening assay”) for identifying modulators, i.e., candidate or test compounds or agents (e.g., peptides, peptidomimetics, small molecules or other drugs) which bind to HNMDA-1 polypeptides, have a stimulatory or inhibitory effect on, for example, HNMDA-1 expression or HNMDA-1 activity, or have a stimulatory or inhibitory effect on, for example, the expression or activity of HNMDA-1 substrate.

In one embodiment, the invention provides assays for screening candidate or test compounds which are substrates of an HNMDA-1 polypeptide or polypeptide or biologically active portion thereof. In another embodiment, the invention provides assays for screening candidate or test compounds which bind to or modulate the activity of an HNMDA-1 polypeptide or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an HNMDA-1 polypeptide or biologically active portion thereof is contacted with a test compound and the ability of the test compound to modulate HNMDA-1 activity is determined. Determining the ability of the test compound to modulate HNMDA-1 activity can be accomplished by monitoring, for example, intracellular or extracellular L-glutamate, glycine, or Ca²⁺ concentration. The cell, for example, can be of mammalian origin, e.g., a neural cell.

The ability of the test compound to modulate HNMDA-1 binding to a substrate or to bind to HNMDA-1 can also be determined. Determining the ability of the test compound to modulate HNMDA-1 binding to a substrate can be accomplished, for example, by coupling the HNMDA-1 substrate with a radioisotope or enzymatic label such that binding of the HNMDA-1 substrate to HNMDA-1 can be determined by detecting the labeled HNMDA-1 substrate in a complex. Alternatively, HNMDA-1 could be coupled with a radioisotope or enzymatic label to monitor the ability of a test compound to modulate HNMDA-1 binding to an HNMDA-1 substrate in a complex. Determining the ability of the test compound to bind HNMDA-1 can be accomplished, for example, by coupling the compound with a radioisotope or enzymatic label such that binding of the compound to HNMDA-1 can be determined by detecting the labeled HNMDA-1 compound in a complex. For example, compounds (e.g., HNMDA-1 substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., an HNMDA-1 substrate) to interact with HNMDA-1 without the labeling of any of the interactants. For example, a microphysiometer can be used to detect the interaction of a compound with HNMDA-1 without the labeling of either the compound or the HNMDA-1. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and HNMDA-1.

In another embodiment, an assay is a cell-based assay comprising contacting a cell expressing an HNMDA-1 target molecule (e.g., an HNMDA-1 substrate) with a test compound and determining the ability of the test compound to modulate (e.g., stimulate or inhibit) the activity of the HNMDA-1 target molecule. Determining the ability of the test compound to modulate the activity of an HNMDA-1 target molecule can be accomplished, for example, by determining the ability of the HNMDA-1 polypeptide to bind to or interact with the HNMDA-1 target molecule.

Determining the ability of the HNMDA-1 polypeptide, or a biologically active fragment thereof, to bind to or interact with an HNMDA-1 target molecule can be accomplished by one of the methods described above for determining direct binding. In a preferred embodiment, determining the ability of the HNMDA-1 polypeptide to bind to or interact with an HNMDA-1 target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target using an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which an HNMDA-1 polypeptide or biologically active portion thereof is contacted with a test compound and the ability of the test compound to bind to the HNMDA-1 polypeptide or biologically active portion thereof is determined. Preferred biologically active portions of the HNMDA-1 polypeptides to be used in assays of the present invention include fragments which participate in interactions with non-HNMDA-1 molecules, e.g., fragments with high surface probability scores (see, for example, FIG. 33). Binding of the test compound to the HNMDA-1 polypeptide can be determined either directly or indirectly as described above. In a preferred embodiment, the assay includes contacting the HNMDA-1 polypeptide or biologically active portion thereof with a known compound which binds HNMDA-1 to form an assay mixture, contacting the assay mixture with a test compound, and determining the ability of the test compound to interact with an HNMDA-1 polypeptide, wherein determining the ability of the test compound to interact with an HNMDA-1 polypeptide comprises determining the ability of the test compound to preferentially bind to HNMDA-1 or biologically active portion thereof as compared to the known compound.

In another embodiment, the assay is a cell-free assay in which an HNMDA-1 polypeptide or biologically active portion thereof is contacted with a test compound and the ability of the test compound to modulate (e.g., stimulate or inhibit) the activity of the HNMDA-1 polypeptide or biologically active portion thereof is determined. Determining the ability of the test compound to modulate the activity of an HNMDA-1 polypeptide can be accomplished, for example, by determining the ability of the HNMDA-1 polypeptide to bind to an HNMDA-1 target molecule by one of the methods described above for determining direct binding. Determining the ability of the HNMDA-1 polypeptide to bind to an HNMDA-1 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an HNMDA-1 polypeptide can be accomplished by determining the ability of the HNMDA-1 polypeptide to further modulate the activity of a downstream effector of an HNMDA-1 target molecule. For example, the activity of the effector molecule on an appropriate target can be determined or the binding of the effector to an appropriate target can be determined as previously described.

In yet another embodiment, the cell-free assay involves contacting an HNMDA-1 polypeptide or biologically active portion thereof with a known compound which binds the HNMDA-1 polypeptide to form an assay mixture, contacting the assay mixture with a test compound, and determining the ability of the test compound to interact with the HNMDA-1 polypeptide, wherein determining the ability of the test compound to interact with the HNMDA-1 polypeptide comprises determining the ability of the HNMDA-1 polypeptide to preferentially bind to or modulate the activity of an HNMDA-1 target molecule.

In more than one embodiment of the above assay methods of the present invention, it may be desirable to immobilize either HNMDA-1 or its target molecule to facilitate separation of complexed from uncomplexed forms of one or both of the proteins, as well as to accommodate automation of the assay. Binding of a test compound to an HNMDA-1 polypeptide, or interaction of an HNMDA-1 polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/HNMDA-1 fusion proteins or glutathione-S-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or HNMDA-1 polypeptide, and the mixture incubated under conditions conducive to complex formation (e.g., at physiological conditions for salt and pH). Following incubation, the beads or micrometer plate wells are washed to remove any unbound components, the matrix immobilized in the case of beads, complex determined either directly or indirectly, for example, as described above. Alternatively, the complexes can be dissociated from the matrix, and the level of HNMDA-1 binding or activity determined using standard techniques.

Other techniques for immobilizing proteins on matrices can also be used in the screening assays of the invention. For example, either an HNMDA-1 polypeptide or an HNMDA-1 target molecule can be immobilized utilizing conjugation of biotin and streptavidin. Biotinylated HNMDA-1 polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with HNMDA-1 polypeptide or target molecules but which do not interfere with binding of the HNMDA-1 polypeptide to its target molecule can be derivatized to the wells of the plate, and unbound target or HNMDA-1 polypeptide trapped in the wells by antibody conjugation. Methods for detecting such complexes, in addition to those described above for the GST-immobilized complexes, include immunodetection of complexes using antibodies reactive with the HNMDA-1 polypeptide or target molecule, as well as enzyme-linked assays which rely on detecting an enzymatic activity associated with the HNMDA-1 polypeptide or target molecule.

In another embodiment, modulators of HNMDA-1 expression are identified in a method wherein a cell is contacted with a candidate compound and the expression of HNMDA-1 mRNA or polypeptide in the cell is determined. The level of expression of HNMDA-1 mRNA or polypeptide in the presence of the candidate compound is compared to the level of expression of HNMDA-1 mRNA or polypeptide in the absence of the candidate compound. The candidate compound can then be identified as a modulator of HNMDA-1 expression based on this comparison. For example, when expression of HNMDA-1 mRNA or polypeptide is greater (statistically significantly greater) in the presence of the candidate compound than in its absence, the candidate compound is identified as a stimulator of HNMDA-1 mRNA or polypeptide expression. Alternatively, when expression of HNMDA-1 mRNA or polypeptide is less (statistically significantly less) in the presence of the candidate compound than in its absence, the candidate compound is identified as an inhibitor of HNMDA-1 mRNA or polypeptide expression. The level of HNMDA-1 mRNA or polypeptide expression in the cells can be determined by methods described herein for detecting HNMDA-1 mRNA or polypeptide.

In yet another aspect of the invention, the HNMDA-1 polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with HNMDA-1 (“HNMDA-1-binding proteins” or “HNMDA-1-bp”) and are involved in HNMDA-1 activity. Such HNMDA-1-binding proteins are also likely to be involved in the propagation of signals by the HNMDA-1 polypeptides or HNMDA-1 targets as, for example, downstream elements of an HNMDA-1-mediated signaling pathway. Alternatively, such HNMDA-1-binding proteins are likely to be HNMDA-1 inhibitors.

The two-hybrid system is based on the modular nature of most transcription factors, which consist of separable DNA-binding and activation domains. Briefly, the assay utilizes two different DNA constructs. In one construct, the gene that codes for an HNMDA-1 polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an HNMDA-1-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the HNMDA-1 polypeptide.

In another aspect, the invention pertains to a combination of two or more of the assays described herein. For example, a modulating agent can be identified using a cell-based or a cell free assay, and the ability of the agent to modulate the activity of an HNMDA-1 polypeptide can be confirmed in vivo, e.g., in an animal such as an animal model for a learning or memory disorder.

This invention further pertains to novel agents identified by the above-described screening assays. Accordingly, it is within the scope of this invention to further use an agent identified as described herein in an appropriate animal model. For example, an agent identified as described herein (e.g., an HNMDA-1 modulating agent, an antisense HNMDA-1 nucleic acid molecule, an HNMDA-1-specific antibody, or an HNMDA-1-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the HNMDA-1 nucleotide sequences, described herein, can be used to map the location of the HNMDA-1 genes on a chromosome. The mapping of the HNMDA-1 sequences to chromosomes is an important first step in correlating these sequences with genes associated with disease.

Briefly, HNMDA-1 genes can be mapped to chromosomes by preparing PCR primers (preferably 15-25 bp in length) from the HNMDA-1 nucleotide sequences. Computer analysis of the HNMDA-1 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the HNMDA-1 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the HNMDA-1 nucleotide sequences to design oligonucleotide primers, sublocalization can be achieved with panels of fragments from specific chromosomes. Other mapping strategies which can similarly be used to map an HNMDA-1 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the HNMDA-1 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The HNMDA-1 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the HNMDA-1 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The HNMDA-1 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:37 can comfortably provide positive individual identification with a panel of perhaps 10 to 1,000 primers which each yield a noncoding amplified sequence of 100 bases. If predicted coding sequences, such as those in SEQ ID NO:39 are used, a more appropriate number of primers for positive individual identification would be 500-2,000.

If a panel of reagents from HNMDA-1 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of HNMDA-1 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:37 are particularly appropriate for this use as greater numbers of polymorphisms occur in the noncoding regions, making it easier to differentiate individuals using this technique. Examples of polynucleotide reagents include the HNMDA-1 nucleotide sequences or portions thereof, e.g., fragments derived from the noncoding regions of SEQ ID NO:37 having a length of at least 20 bases, preferably at least 30 bases.

The HNMDA-1 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such HNMDA-1 probes can be used to identify tissue by species and/or by organ type.

In a similar fashion, these reagents, e.g., HNMDA-1 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining HNMDA-1 polypeptide and/or nucleic acid expression as well as HNMDA-1 activity, in the context of a biological sample (e.g., blood, serum, cells, tissue) to thereby determine whether an individual is afflicted with a disease or disorder, or is at risk of developing a disorder, associated with aberrant or unwanted HNMDA-1 expression or activity. The invention also provides for prognostic (or predictive) assays for determining whether an individual is at risk of developing a disorder associated with HNMDA-1 polypeptide, nucleic acid expression or activity. For example, mutations in an HNMDA-1 gene can be assayed in a biological sample. Such assays can be used for prognostic or predictive purpose to thereby prophylactically treat an individual prior to the onset of a disorder characterized by or associated with HNMDA-1 polypeptide, nucleic acid expression or activity.

Another aspect of the invention pertains to monitoring the influence of agents (e.g., drugs, compounds) on the expression or activity of HNMDA-1 in clinical trials.

These and other agents are described in further detail in the following sections.

1. Diagnostic Assays

An exemplary method for detecting the presence or absence of HNMDA-1 polypeptide or nucleic acid in a biological sample involves obtaining a biological sample from a test subject and contacting the biological sample with a compound or an agent capable of detecting HNMDA-1 polypeptide or nucleic acid (e.g., mRNA, or genomic DNA) that encodes HNMDA-1 polypeptide such that the presence of HNMDA-1 polypeptide or nucleic acid is detected in the biological sample. In another aspect, the present invention provides a method for detecting the presence of HNMDA-1 activity in a biological sample by contacting the biological sample with an agent capable of detecting an indicator of HNMDA-1 activity such that the presence of HNMDA-1 activity is detected in the biological sample. A preferred agent for detecting HNMDA-1 mRNA or genomic DNA is a labeled nucleic acid probe capable of hybridizing to HNMDA-1 mRNA or genomic DNA. The nucleic acid probe can be, for example, the HNMDA-1 nucleic acid set forth in SEQ ID NO:37 or 39, or the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to HNMDA-1 mRNA or genomic DNA. Other suitable probes for use in the diagnostic assays of the invention are described herein.

A preferred agent for detecting HNMDA-1 polypeptide is an antibody capable of binding to HNMDA-1 polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect HNMDA-1 mRNA, polypeptide, or genomic DNA in a biological sample in vitro as well as in vivo. For example, in vitro techniques for detection of HNMDA-1 mRNA include Northern hybridizations and in situ hybridizations. In vitro techniques for detection of HNMDA-1 polypeptide include enzyme linked immunosorbent assays (ELISAs), Western blots, immunoprecipitations and immunofluorescence. In vitro techniques for detection of HNMDA-1 genomic DNA include Southern hybridizations. Furthermore, in vivo techniques for detection of HNMDA-1 polypeptide include introducing into a subject a labeled anti-HNMDA-1 antibody. For example, the antibody can be labeled with a radioactive marker whose presence and location in a subject can be detected by standard imaging techniques.

The present invention also provides diagnostic assays for identifying the presence or absence of a genetic alteration characterized by at least one of (i) aberrant modification or mutation of a gene encoding an HNMDA-1 polypeptide; (ii) aberrant expression of a gene encoding an HNMDA-1 polypeptide; (iii) mis-regulation of the gene; and (iii) aberrant post-translational modification of an HNMDA-1 polypeptide, wherein a wild-type form of the gene encodes a polypeptide with an HNMDA-1 activity. “Misexpression or aberrant expression”, as used herein, refers to a non-wild type pattern of gene expression, at the RNA or protein level. It includes, but is not limited to, expression at non-wild type levels (e.g., over or under expression); a pattern of expression that differs from wild type in terms of the time or stage at which the gene is expressed (e.g., increased or decreased expression (as compared with wild type) at a predetermined developmental period or stage); a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene (e.g., a pattern of increased or decreased expression (as compared with wild type) in the presence of an increase or decrease in the strength of the stimulus).

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting HNMDA-1 polypeptide, mRNA, or genomic DNA, such that the presence of HNMDA-1 polypeptide, mRNA or genomic DNA is detected in the biological sample, and comparing the presence of HNMDA-1 polypeptide, mRNA or genomic DNA in the control sample with the presence of HNMDA-1 polypeptide, mRNA or genomic DNA in the test sample.

The invention also encompasses kits for detecting the presence of HNMDA-1 in a biological sample. For example, the kit can comprise a labeled compound or agent capable of detecting HNMDA-1 polypeptide or mRNA in a biological sample; means for determining the amount of HNMDA-1 in the sample; and means for comparing the amount of HNMDA-1 in the sample with a standard. The compound or agent can be packaged in a suitable container. The kit can further comprise instructions for using the kit to detect HNMDA-1 polypeptide or nucleic acid.

2. Prognostic Assays

The diagnostic methods described herein can furthermore be utilized to identify subjects having or at risk of developing a disease or disorder associated with aberrant or unwanted HNMDA-1 expression or activity. As used herein, the term “aberrant” includes an HNMDA-1 expression or activity which deviates from the wild type HNMDA-1 expression or activity. Aberrant expression or activity includes increased or decreased expression or activity, as well as expression or activity which does not follow the wild type developmental pattern of expression or the subcellular pattern of expression. For example, aberrant HNMDA-1 expression or activity is intended to include the cases in which a mutation in the HNMDA-1 gene causes the HNMDA-1 gene to be under-expressed or over-expressed and situations in which such mutations result in a non-functional HNMDA-1 polypeptide or a polypeptide which does not function in a wild-type fashion, e.g., a polypeptide which does not interact with an HNMDA-1 substrate, e.g., a glutamate-gated ion channel family member subunit or ligand, or one which interacts with a non-HNMDA-1 substrate, e.g. a non-glutamate-gated ion channel family member subunit or ligand. As used herein, the term “unwanted” includes an unwanted phenomenon involved in a biological response, such as cellular proliferation. For example, the term unwanted includes an HNMDA-1 expression or activity which is undesirable in a subject.

The assays described herein, such as the preceding diagnostic assays or the following assays, can be utilized to identify a subject having or at risk of developing a disorder associated with a misregulation in HNMDA-1 polypeptide activity or nucleic acid expression, such as a glutamate-gated ion channel disorder. Alternatively, the prognostic assays can be utilized to identify a subject having or at risk for developing a disorder associated with a misregulation in HNMDA-1 polypeptide activity or nucleic acid expression, such as a glutamate-gated ion channel disorder. Thus, the present invention provides a method for identifying a disease or disorder associated with aberrant or unwanted HNMDA-1 expression or activity in which a test sample is obtained from a subject and HNMDA-1 polypeptide or nucleic acid (e.g., mRNA or genomic DNA) is detected, wherein the presence of HNMDA-1 polypeptide or nucleic acid is diagnostic for a subject having or at risk of developing a disease or disorder associated with aberrant or unwanted HNMDA-1 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted HNMDA-1 expression or activity. For example, such methods can be used to determine whether a subject can be effectively treated with an agent for a glutamate-gated ion channel disorder. Thus, the present invention provides methods for determining whether a subject can be effectively treated with an agent for a disorder associated with aberrant or unwanted HNMDA-1 expression or activity in which a test sample is obtained and HNMDA-1 polypeptide or nucleic acid expression or activity is detected (e.g., wherein the abundance of HNMDA-1 polypeptide or nucleic acid expression or activity is diagnostic for a subject that can be administered the agent to treat a disorder associated with aberrant or unwanted HNMDA-1 expression or activity).

The methods of the invention can also be used to detect genetic alterations in an HNMDA-1 gene, thereby determining if a subject with the altered gene is at risk for a disorder characterized by misregulation in HNMDA-1 polypeptide activity or nucleic acid expression, such as a glutamate-gated ion channel disorder. In preferred embodiments, the methods include detecting, in a sample of cells from the subject, the presence or absence of a genetic alteration characterized by at least one of an alteration affecting the integrity of a gene encoding an HNMDA-1-polypeptide, or the mis-expression of the HNMDA-1 gene. For example, such genetic alterations can be detected by ascertaining the existence of at least one of 1) a deletion of one or more nucleotides from an HNMDA-1 gene; 2) an addition of one or more nucleotides to an HNMDA-1 gene; 3) a substitution of one or more nucleotides of an HNMDA-1 gene, 4) a chromosomal rearrangement of an HNMDA-1 gene; 5) an alteration in the level of a messenger RNA transcript of an HNMDA-1 gene, 6) aberrant modification of an HNMDA-1 gene, such as of the methylation pattern of the genomic DNA, 7) the presence of a non-wild type splicing pattern of a messenger RNA transcript of an HNMDA-1 gene, 8) a non-wild type level of an HNMDA-1-polypeptide, 9) allelic loss of an HNMDA-1 gene, and 10) inappropriate post-translational modification of an HNMDA-1-polypeptide. As described herein, there are a large number of assays known in the art which can be used for detecting alterations in an HNMDA-1 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the HNMDA-1-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to an HNMDA-1 gene under conditions such that hybridization and amplification of the HNMDA-1-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an HNMDA-1 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in HNMDA-1 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in HNMDA-1 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the HNMDA-1 gene and detect mutations by comparing the sequence of the sample HNMDA-1 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the HNMDA-1 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type HNMDA-1 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in HNMDA-1 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an HNMDA-1 sequence, e.g., a wild-type HNMDA-1 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in HNMDA-1 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control HNMDA-1 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an HNMDA-1 gene.

Furthermore, any cell type or tissue in which HNMDA-1 is expressed may be utilized in the prognostic assays described herein.

3. Monitoring of Effects During Clinical Trials

Monitoring the influence of agents (e.g., drugs) on the expression or activity of an HNMDA-1 polypeptide (e.g., the modulation of Ca²⁺ transport and L-glutamate and/or glycine binding) can be applied not only in basic drug screening, but also in clinical trials. For example, the effectiveness of an agent determined by a screening assay as described herein to increase HNMDA-1 gene expression, polypeptide levels, or upregulate HNMDA-1 activity, can be monitored in clinical trials of subjects exhibiting decreased HNMDA-1 gene expression, polypeptide levels, or downregulated HNMDA-1 activity. Alternatively, the effectiveness of an agent determined by a screening assay to decrease HNMDA-1 gene expression, polypeptide levels, or downregulate HNMDA-1 activity, can be monitored in clinical trials of subjects exhibiting increased HNMDA-1 gene expression, polypeptide levels, or upregulated HNMDA-1 activity. In such clinical trials, the expression or activity of an HNMDA-1 gene, and preferably, other genes that have been implicated in, for example, an HNMDA-1-associated disorder can be used as a “read out” or markers of the phenotype of a particular cell.

For example, and not by way of limitation, genes, including HNMDA-1, that are modulated in cells by treatment with an agent (e.g., compound, drug or small molecule) which modulates HNMDA-1 activity (e.g., identified in a screening assay as described herein) can be identified. Thus, to study the effect of agents on glutamate-gated ion channel-associated disorders (e.g., disorders characterized by deregulated long term potentiation or Ca²⁺ transport), for example, in a clinical trial, cells can be isolated and RNA prepared and analyzed for the levels of expression of HNMDA-1 and other genes implicated in the glutamate-gated ion channel-associated disorder, respectively. The levels of gene expression (e.g., a gene expression pattern) can be quantified by northern blot analysis or RT-PCR, as described herein, or alternatively by measuring the amount of polypeptide produced, by one of the methods as described herein, or by measuring the levels of activity of HNMDA-1 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an HNMDA-1 polypeptide, mRNA, or genomic DNA in the preadministration sample; (iii) obtaining one or more post-administration samples from the subject; (iv) detecting the level of expression or activity of the HNMDA-1 polypeptide, mRNA, or genomic DNA in the post-administration samples; (v) comparing the level of expression or activity of the HNMDA-1 polypeptide, mRNA, or genomic DNA in the pre-administration sample with the HNMDA-1 polypeptide, mRNA, or genomic DNA in the post administration sample or samples; and (vi) altering the administration of the agent to the subject accordingly. For example, increased administration of the agent may be desirable to increase the expression or activity of FINMDA-1 to higher levels than detected, i.e., to increase the effectiveness of the agent. Alternatively, decreased administration of the agent may be desirable to decrease expression or activity of HNMDA-1 to lower levels than detected, i.e. to decrease the effectiveness of the agent. According to such an embodiment, HNMDA-1 expression or activity may be used as an indicator of the effectiveness of an agent, even in the absence of an observable phenotypic response.

4. Methods of Treatment:

The present invention provides for both prophylactic and therapeutic methods of treating a subject at risk of (or susceptible to) a disorder or having a disorder associated with aberrant or unwanted HNMDA-1 expression or activity, e.g. a glutamate-gated ion channel disorder. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the HNMDA-1 molecules of the present invention or HNMDA-1 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

2. Prophylactic Methods

In one aspect, the invention provides a method for preventing in a subject, a disease or condition associated with an aberrant or unwanted HNMDA-1 expression or activity, by administering to the subject an HNMDA-1 or an agent which modulates HNMDA-1 expression or at least one HNMDA-1 activity. Subjects at risk for a disease which is caused or contributed to by aberrant or unwanted HNMDA-1 expression or activity can be identified by, for example, any or a combination of diagnostic or prognostic assays as described herein. Administration of a prophylactic agent can occur prior to the manifestation of symptoms characteristic of the HNMDA-1 aberrancy, such that a disease or disorder is prevented or, alternatively, delayed in its progression. Depending on the type of HNMDA-1 aberrancy, for example, an HNMDA-1 agonist or HNMDA-1 antagonist agent can be used for treating the subject. The appropriate agent can be determined based on screening assays described herein.

2. Therapeutic Methods

Another aspect of the invention pertains to methods of modulating HNMDA-1 expression or activity for therapeutic purposes. Accordingly, in an exemplary embodiment, the modulatory method of the invention involves contacting a cell capable of expressing HNMDA-1 with an agent that modulates one or more of the activities of HNMDA-1 polypeptide activity associated with the cell, such that HNMDA-1 activity in the cell is modulated. An agent that modulates HNMDA-1 polypeptide activity can be an agent as described herein, such as a nucleic acid or a polypeptide, a naturally-occurring target molecule of an HNMDA-1 polypeptide (e.g., an HNMDA-1 substrate), an HNMDA-1 antibody, an HNMDA-1 agonist or antagonist, a peptidomimetic of an HNMDA-1 agonist or antagonist, or other small molecule. In one embodiment, the agent stimulates one or more HNMDA-1 activities. Examples of such stimulatory agents include active HNMDA-1 polypeptide and a nucleic acid molecule encoding HNMDA-1 that has been introduced into the cell. In another embodiment, the agent inhibits one or more HNMDA-1 activities. Examples of such inhibitory agents include antisense HNMDA-1 nucleic acid molecules, anti-HNMDA-1 antibodies, and HNMDA-1 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of an HNMDA-1 polypeptide or nucleic acid molecule. In one embodiment, the method involves administering an agent (e.g., an agent identified by a screening assay described herein), or combination of agents that modulates (e.g., upregulates or downregulates) HNMDA-1 expression or activity. In another embodiment, the method involves administering an HNMDA-1 polypeptide or nucleic acid molecule as therapy to compensate for reduced, aberrant, or unwanted HNMDA-1 expression or activity.

Stimulation of HNMDA-1 activity is desirable in situations in which HNMDA-1 is abnormally downregulated and/or in which increased HNMDA-1 activity is likely to have a beneficial effect. Likewise, inhibition of HNMDA-1 activity is desirable in situations in which HNMDA-1 is abnormally upregulated and/or in which decreased HNMDA-1 activity is likely to have a beneficial effect.

3. Pharmacogenomics

The HNMDA-1 molecules of the present invention, as well as agents, or modulators which have a stimulatory or inhibitory effect on HNMDA-1 activity (e.g., HNMDA-1 gene expression) as identified by a screening assay described herein can be administered to individuals to treat (prophylactically or therapeutically) glutamate-gated ion channel-associated disorders (e.g., learning or memory disorders) associated with aberrant or unwanted HNMDA-1 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an HNMDA-1 molecule or HNMDA-1 modulator as well as tailoring the dosage and/or therapeutic regimen of treatment with an HNMDA-1 molecule or HNMDA-1 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., an HNMDA-1 polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an HNMDA-1 molecule or HNMDA-1 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an HNMDA-1 molecule or HNMDA-1 modulator, such as a modulator identified by one of the exemplary screening assays described herein.

4. Use of HNMDA-1 Molecules as Surrogate Markers

The HNMDA-1 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the HNMDA-1 molecules of the invention may be detected, and may be correlated with one or more biological states in vivo. For example, the HNMDA-1 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The HNMDA-1 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., an HNMDA-1 marker) transcription or expression, the amplified marker may be in a quantity which is more readily detectable than the drug itself. Also, the marker may be more easily detected due to the nature of the marker itself; for example, using the methods described herein, anti-HNMDA-1 antibodies may be employed in an immune-based detection system for an HNMDA-1 polypeptide marker, or HNMDA-1-specific radiolabeled probes may be used to detect an HNMDA-1 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The HNMDA-1 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or polypeptide (e.g., HNMDA-1 polypeptide or RNA) for specific tumor markers in a subject, a drug or course of treatment may be selected that is optimized for the treatment of the specific tumor likely to be present in the subject. Similarly, the presence or absence of a specific sequence mutation in HNMDA-1 DNA may correlate HNMDA-1 drug response. The use of pharmacogenomic markers therefore permits the application of the most appropriate treatment for each subject without having to administer the therapy.

5. Electronic Apparatus Readable Media and Arrays

Electronic apparatus readable media comprising HNMDA-1 sequence information is also provided. As used herein, “HNMDA-1 sequence information” refers to any nucleotide and/or amino acid sequence information particular to the HNMDA-1 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said HNMDA-1 sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding, or containing data or information that can be read and accessed directly by an electronic apparatus. Such media can include, but are not limited to: magnetic storage media, such as floppy discs, hard disc storage medium, and magnetic tape; optical storage media such as compact discs; electronic storage media such as RAM, ROM, EPROM, EEPROM and the like; and general hard disks and hybrids of these categories such as magnetic/optical storage media. The medium is adapted or configured for having recorded thereon HNMDA-1 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatuses; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the HNMDA-1 sequence information. A variety of software programs and formats can be used to store the sequence information on the electronic apparatus readable medium. For example, the sequence information can be represented in a word processing text file, formatted in commercially-available software such as WordPerfect and Microsoft Word, represented in the form of an ASCII file, or stored in a database application, such as DB2, Sybase, Oracle, or the like, as well as in other forms. Any number of dataprocessor structuring formats (e.g., text file or database) may be employed in order to obtain or create a medium having recorded thereon the HNMDA-1 sequence information.

By providing HNMDA-1 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a HNMDA-1 associated disease or disorder or a pre-disposition to a HNMDA-1 associated disease or disorder, wherein the method comprises the steps of determining HNMDA-1 sequence information associated with the subject and based on the HNMDA-1 sequence information, determining whether the subject has a HNMDA-1 associated disease or disorder or a pre-disposition to a HNMDA-1 associated disease or disorder, and/or recommending a particular treatment for the disease, disorder, or pre-disease condition.

The present invention further provides in an electronic system and/or in a network, a method for determining whether a subject has a HNMDA-1 associated disease or disorder or a pre-disposition to a disease associated with HNMDA-1 wherein the method comprises the steps of determining HNMDA-1 sequence information associated with the subject, and based on the HNMDA-1 sequence information, determining whether the subject has a HNMDA-1 associated disease or disorder or a pre-disposition to a HNMDA-1 associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a HNMDA-1 associated disease or disorder or a pre-disposition to a HNMDA-1 associated disease or disorder associated with HNMDA-1, said method comprising the steps of receiving HNMDA-1 sequence information from the subject and/or information related thereto, receiving phenotypic information associated with the subject, acquiring information from the network corresponding to HNMDA-1 and/or a HNMDA-1 associated disease or disorder, and based on one or more of the phenotypic information, the HNMDA-1 information (e.g., sequence information and/or information related thereto), and the acquired information, determining whether the subject has a HNMDA-1 associated disease or disorder or a pre-disposition to a HNMDA-1 associated disease or disorder. The method may further comprise the step of recommending a particular treatment for the disease, disorder or pre-disease condition.

The present invention also provides a business method for determining whether a subject has a HNMDA-1 associated disease or disorder or a pre-disposition to a HNMDA-1 associated disease or disorder, said method comprising the steps of receiving information related to HNMDA-1 (e.g., sequence information and/or information related thereto), receiving phenotypic information associated with the subject, acquiring information from the network related to HNMDA-1 and/or related to a HNMDA-1 associated disease or disorder, and based on one or more of the phenotypic information, the HNMDA-1 information, and the acquired information, determining whether the subject has a HNMDA-1 associated disease or disorder or a pre-disposition to a HNMDA-1 associated disease or disorder. The method may further comprise the step of recommending a particular treatment for the disease, disorder or pre-disease condition.

The invention also includes an array comprising a HNMDA-1 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be HNMDA-1. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a HNMDA-1 associated disease or disorder, progression of HNMDA-1 associated disease or disorder, and processes, such a cellular transformation associated with the HNMDA-1 associated disease or disorder.

The array is also useful for ascertaining the effect of the expression of a gene on the expression of other genes in the same cell or in different cells (e.g., ascertaining the effect of HNMDA-1 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including HNMDA-1) that could serve as a molecular target for diagnosis or therapeutic intervention.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human HNMDA-1 cDNA

In this example, the identification and characterization of the gene encoding human HNMDA-1 (clone 55063) is described.

Isolation of the HNMDA-1 cDNA

The invention is based, at least in part, on the discovery of a human gene encoding a novel polypeptide, referred to herein as HNMDA-1. The entire sequence of the human clone 55063 was determined and found to contain an open reading frame termed human “HNMDA-1.” The nucleotide sequence of the HNMDA-1 gene is set forth in FIG. 32 and in the Sequence Listing as SEQ ID NO:37. The amino acid sequence of the HNMDA-1 expression product is set forth in FIG. 32 and in the Sequence Listing as SEQ ID NO:38. The HNMDA-1 polypeptide comprises 1115 amino acids. The coding region (open reading frame) of SEQ ID NO:37 is set forth as SEQ ID NO:39. Clone 55063, comprising the coding region of HNMDA-1, was deposited with the American Type Culture Collection (ATCC®), 10801 University Boulevard, Manassas, Va. 20110-2209, on ______, and assigned Accession No. ______.

Analysis of the Human HNMDA-1 Molecules

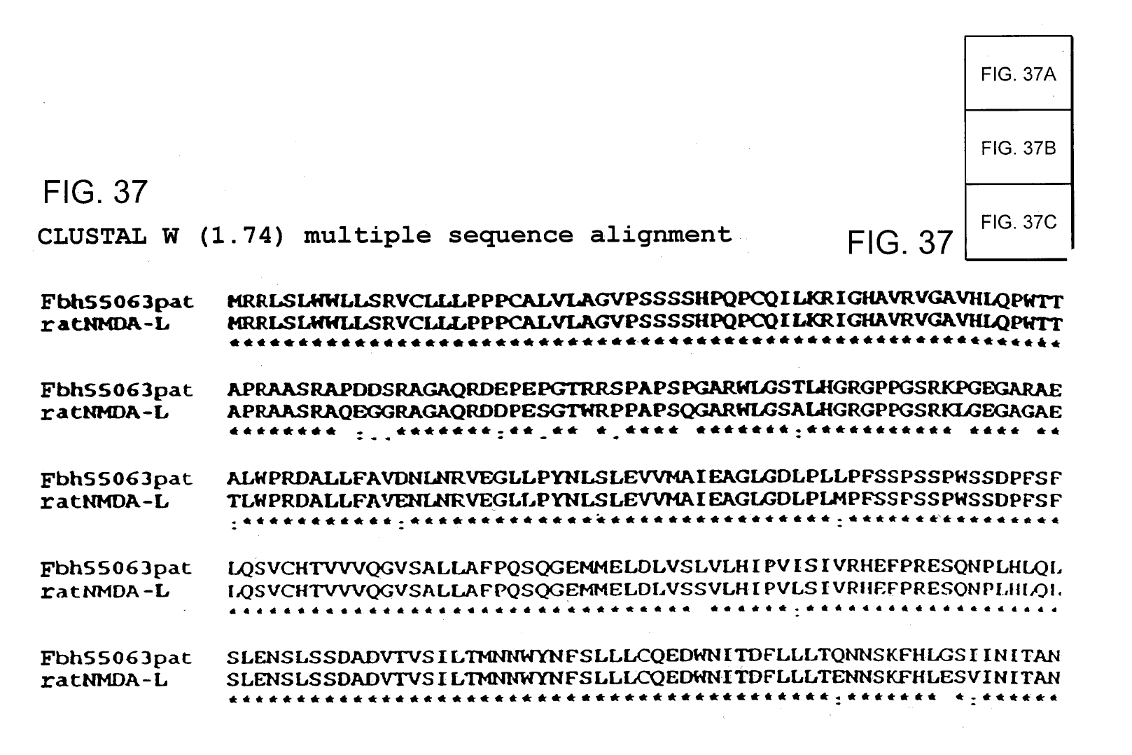

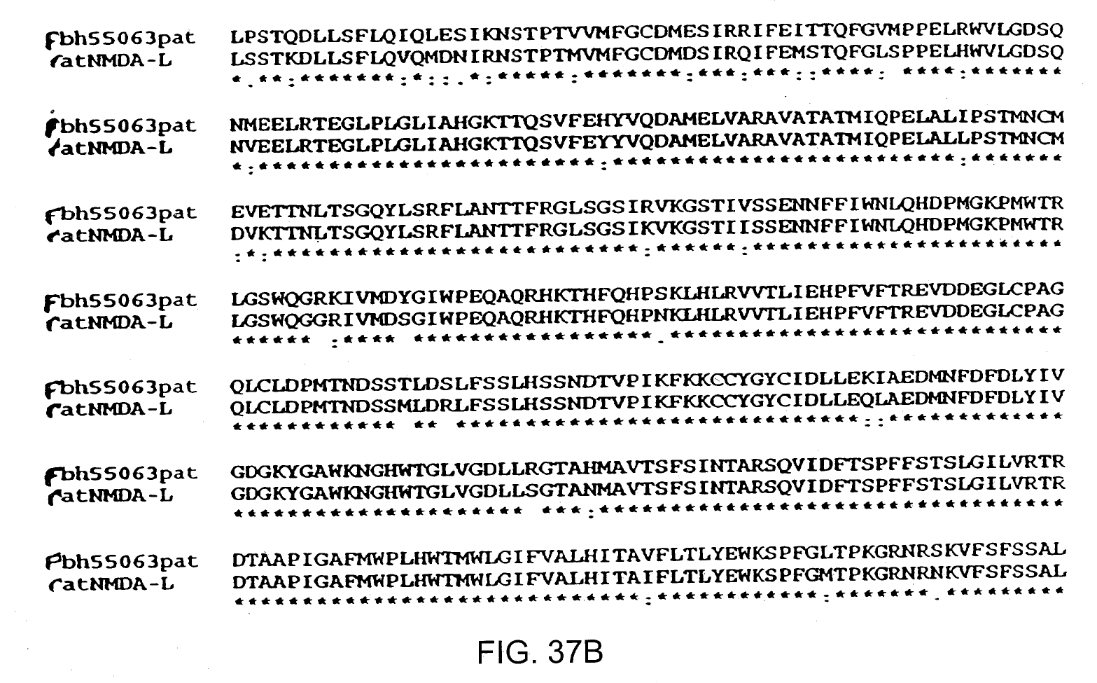

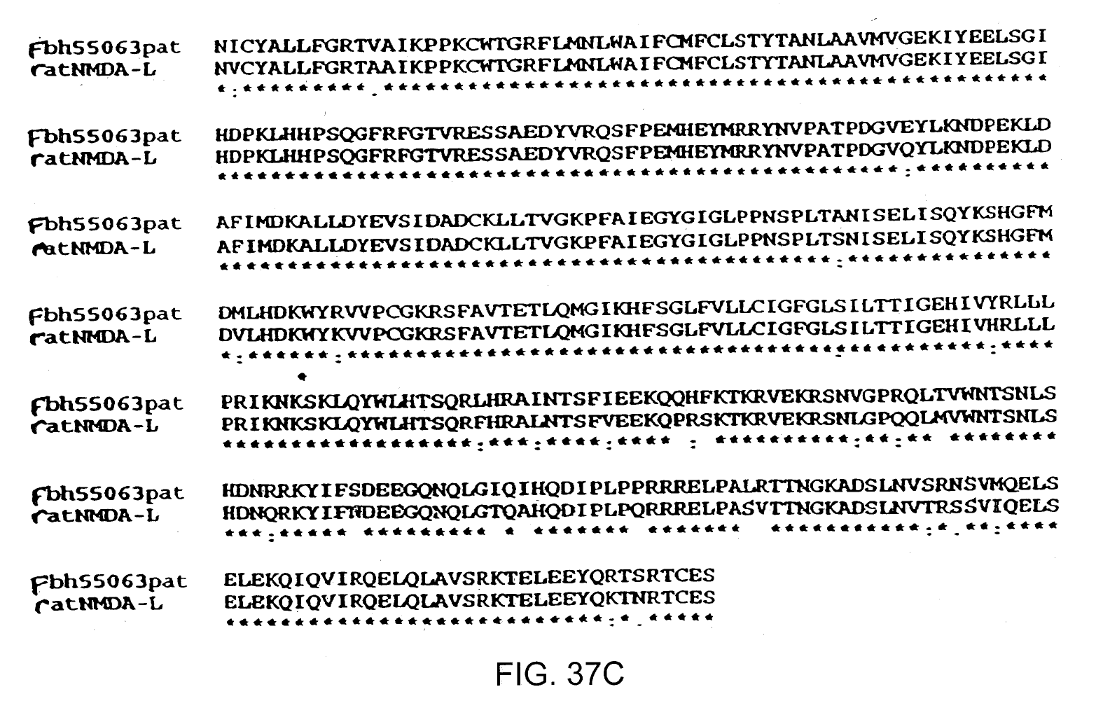

The HNMDA-1 amino acid sequence was aligned with the amino acid sequence of the rat NMDA-L (Accession No. 1050330) amino acid sequence using the CLUSTAL W (1.74) multiple sequence alignment program. The results of the alignment are set forth in FIG. 37.

A search using the polypeptide sequence of SEQ ID NO:38 was performed against the HMM database in PFAM (FIG. 34) resulting in the identification of a potential ligand-gated ion channel family domain in the amino acid sequence of HNMDA-1 at about residues 674-952 of SEQ ID NO:38 (score=198.1).

A search using the polypeptide sequence of SEQ ID NO:38 was also performed against the HMM database in SMART (FIGS. 35A-B) resulting in the identification of a potential glutamate-gated ion channel family domain in the amino acid sequence of HNMDA-1 at about residues 565-910 of SEQ ID NO:38 (score=267.4).

The amino acid sequence of HNMDA-1 was analyzed using the program PSORT (http://www.psort.nibb.ac.jp) to predict the localization of the proteins within the cell. This program assesses the presence of different targeting and localization amino acid sequences within the query sequence. The results of this analysis show that HNMDA-1 may be localized to the endoplasmic reticulum, mitochondria, or nucleus.

Searches of the amino acid sequence of HNMDA-1 were further performed against the Prosite database. These searches resulted in the identification in the amino acid sequence of HNMDA-1 of a number of potential N-glycosylation sites, a potential cAMP- and cGMP-dependent protein kinase phosphorylation site, a number of potential protein kinase C phosphorylation sites, a number of potential casein kinase II phosphorylation sites, a potential tyrosine kinase phosphorylation site, a number of potential N-myristoylation sites, a number of potential amidation sites, and a potential ATP/GTP-binding site motif A (P-loop).

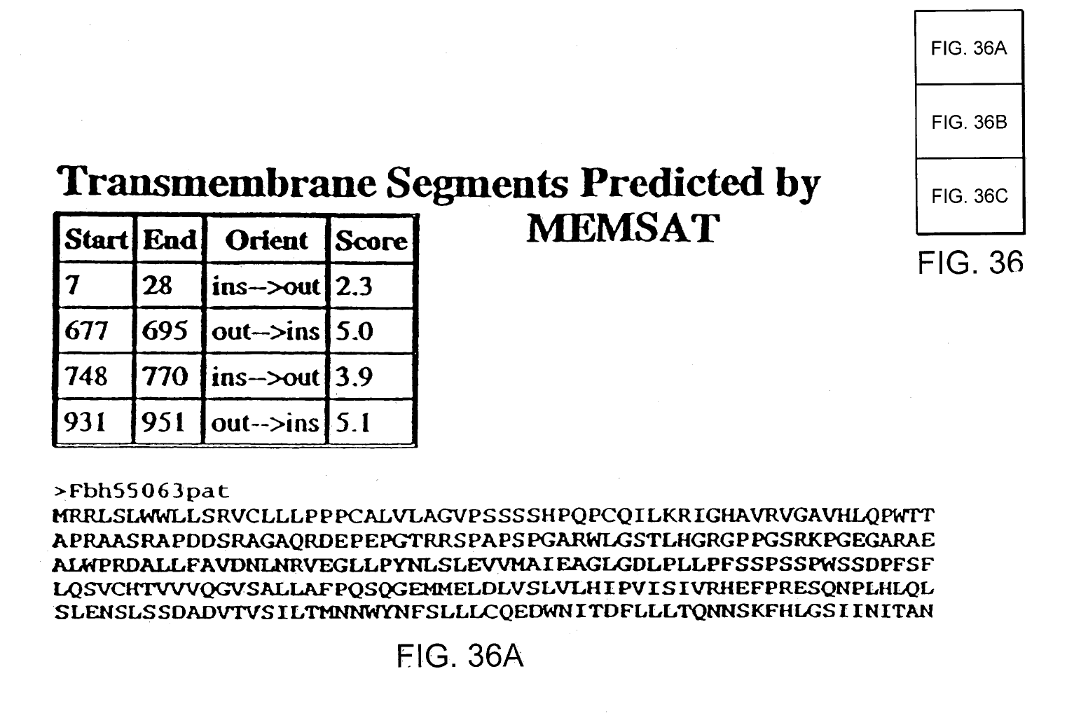

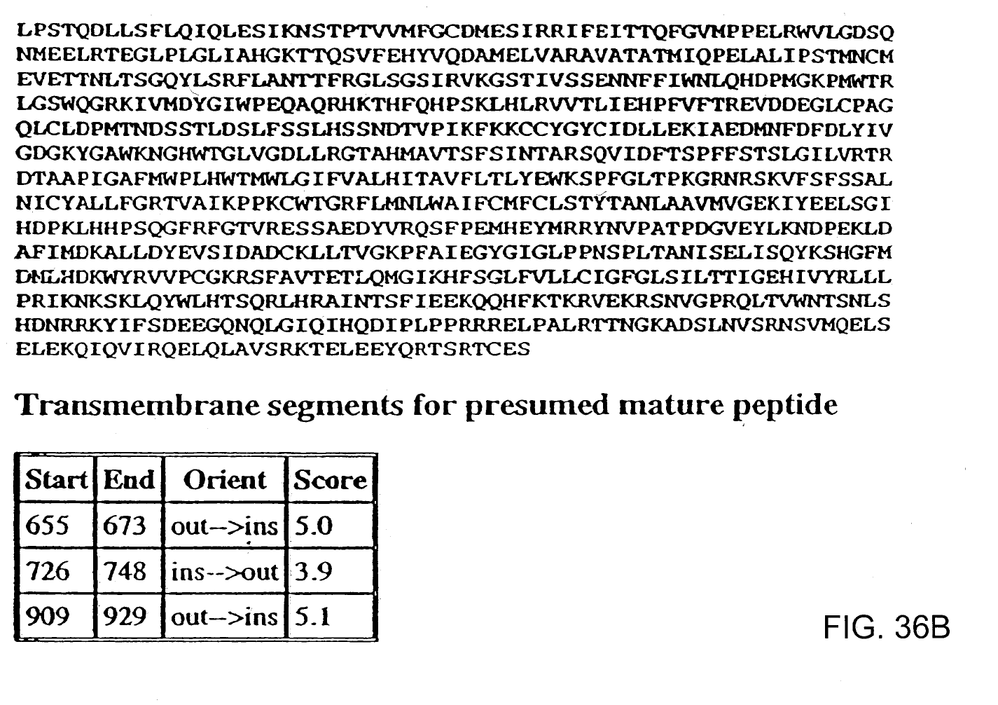

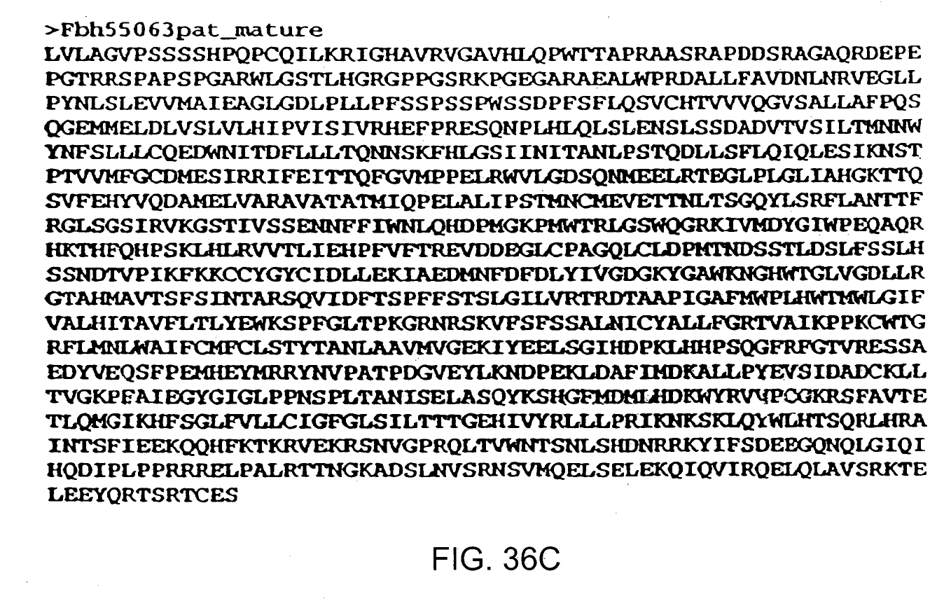

A MEMSAT analysis of the polypeptide sequence of SEQ ID NO:38 was also performed predicting three possible transmembrane domains in the amino acid sequence of HNMDA-1 (SEQ ID NO:38) at about residues 677-695, 748-770, and 931-951 (FIG. 36). Further analysis of the amino acid sequence of SEQ ID NO:38 (e.g., alignment with, for example, a known rat NMDA protein) resulted in the identification of a fourth transmembrane domain at about amino acid residues 713-734 of SEQ ID NO:38.

### Example 2

### Expression of Recombinant Human HNMDA-1 Polypeptide in Bacterial Cells

In this example, human HNMDA-1 is expressed as a recombinant glutathione-S-transferase (GST) fusion polypeptide in E. coli and the fusion polypeptide is isolated and characterized. Specifically, HNMDA-1 is fused to GST and this fusion polypeptide is expressed in E. coli, e.g., strain PEB199. Expression of the GST-HNMDA-1 fusion polypeptide in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant Human HNMDA-1 Polypeptide in COS Cells

To express the human HNMDA-1 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire HNMDA-1 polypeptide and an HA tag (Wilson et al. (1984) Cell 37:767) or a FLAG tag fused in-frame to its 3′ end of the fragment is cloned into the polylinker region of the vector, thereby placing the expression of the recombinant polypeptide under the control of the CMV promoter.

To construct the plasmid, the HNMDA-1 DNA sequence is amplified by PCR using two primers. The 5′ primer contains the restriction site of interest followed by approximately twenty nucleotides of the HNMDA-1 coding sequence starting from the initiation codon; the 3′ end sequence contains complementary sequences to the other restriction site of interest, a translation stop codon, the HA tag or FLAG tag and the last 20 nucleotides of the HNMDA-1 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the HNMDA-1 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the HNMDA-1-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the IC54420 polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the HNMDA-1 coding sequence is cloned directly into the polylinker of the pCDNA/Amp vector using the appropriate restriction sites. The resulting plasmid is transfected into COS cells in the manner described above, and the expression of the HNMDA-1 polypeptide is detected by radiolabeling and immunoprecipitation using an HNMDA-1-specific monoclonal antibody.

### VIII. 56115, A NOVEL HUMAN TWIK POTASSIUM CHANNEL AND USES THEREFOR

### Background of the Invention

Potassium (K⁺) channels are ubiquitous proteins which are involved in the setting of the resting membrane potential as well as in the modulation of the electrical activity of cells. In excitable cells, K⁺ channels influence action potential waveforms, firing frequency, and neurotransmitter secretion (Rudy, B. (1988) Neuroscience, 25, 729-749; Hille, B. (1992) Ionic Channels of Excitable Membranes, 2nd Ed.). In non-excitable cells, they are involved in hormone secretion, cell volume regulation and potentially in cell proliferation and differentiation (Lewis et al. (1995) Annu. Rev. Immunol., 13, 623-653). Developments in electrophysiology have allowed the identification and the characterization of an astonishing variety of K⁺ channels that differ in their biophysical properties, pharmacology, regulation and tissue distribution (Rudy, B. (1988) Neuroscience, 25, 729-749; Hille, B. (1992) Ionic Channels of Excitable Membranes, 2nd Ed.). More recently, cloning efforts have shed considerable light on the mechanisms that determine this functional diversity. Furthermore, analyses of structure-function relationships have provided an important set of data concerning the molecular basis of the biophysical properties (selectivity, gating, assembly) and the pharmacological properties of cloned K⁺ channels.

Functional diversity of K⁺ channels arises mainly from the existence of a great number of genes coding for pore-forming subunits, as well as for other associated regulatory subunits. Two main structural families of pore-forming subunits have been identified. The first one consists of subunits with a conserved hydrophobic core containing six transmembrane domains (TMDs). These K⁺ channel α subunits participate in the formation of outward rectifier voltage-gated (Kv) and Ca²⁺-dependent K⁺ channels. The fourth TMD contains repeated positive charges involved in the voltage gating of these channels and hence in their outward rectification (Logothetis et al. (1992) Neuron, 8, 531-540; Bezanilla et al. (1994) Biophys. J. 66, 1011-1021).

The second family of pore-forming subunits have only two TMDs. They are essential subunits of inward-rectifying (IRK), G-protein-coupled (GIRK) and ATP-sensitive (K_{ATP}) K⁺ channels. The inward rectification results from a voltage-dependent block by cytoplasmic Mg²⁺ and polyamines (Matsuda, H. (1991) Annu. Rev. Physiol., 53, 289-298). A conserved domain, called the P domain, is present in all members of both families (Pongs, O. (1993) J. Membr. Biol., 136, 1-8; Heginbotham et al. (1994) Biophys. J. 66,1061-1067; Mackinnon, R. (1995) Neuron, 14, 889-892; Pascual et al., (1995) Neuron., and 14, 1055-1063). This domain is an essential element of the aqueous K⁺-selective pore. In both groups, the assembly of four subunits is necessary to form a functional K⁺ channel (Mackinnon, R. (1991) Nature, 350, 232-235; Yang et al., (1995) Neuron, 15, 1441-1447.

In both six TMD and two TMD pore-forming subunit families, different subunits coded by different genes can associate to form heterotetramers with new channel properties (Isacoff et al., (1990) Nature, 345, 530-534). A selective formation of heteropolymeric channels may allow each cell to develop the best K⁺ current repertoire suited to its function. Pore-forming α subunits of Kv channels are classified into different subfamilies according to their sequence similarity (Chandy et al. (1993) Trends Pharmacol. Sci., 14: 434). Tetramerization is believed to occur preferentially between members of each subgroup (Covarrubias et al. (1991) Neuron, 7, 763-773). The domain responsible for this selective association is localized in the N-terminal region and is conserved between members of the same subgroup. This domain is necessary for hetero- but not homo-multimeric assembly within a subfamily and prevents co-assembly between subfamilies. Recently, pore-forming subunits with two TMDs were also shown to co-assemble to form heteropolymers (Duprat et al. (1995) Biochem. Biophys. Res. Commun., 212, 657-663. This heteropolymerization seems necessary to give functional GIRKs. IRKs are active as homopolymers but also form heteropolymers.

New structural types of K⁺ channels were identified recently in both humans and yeast. These channels have two P domains in their functional subunit instead of only one (Ketchum et al. (1995) Nature, 376, 690-695; Lesage et al. (1996) J. Biol. Chem, 271, 4183-4187; Lesage et al. (1996) EMBO J., 15, 1004-1011; Reid et al. (1996) Receptors Channels 4, 51-62). The human channel called TWIK-1, has four TMDs. TWIK-1 is expressed widely in human tissues and is particularly abundant in the heart and the brain. TWIK-1 currents are time independent and inwardly rectifying. These properties suggest that TWIK-1 channels are involved in the control of the background K⁺ membrane conductance (Lesage et al. (1996) EMBO J., 15, 1004-1011).

### Summary of the Invention

The present invention is based, at least in part, on the discovery of novel members of the TWIK (for Tandem of P domains in a Weak Inward rectifying K⁺ channel)-like family of potassium channels, referred to herein as TWIK-9 nucleic acid and protein molecules. The TWIK-9 nucleic acid and protein molecules of the present invention are useful as targets for developing modulating agents to regulate a variety of cellular processes. Accordingly, in one aspect, this invention provides isolated nucleic acid molecules encoding TWIK-9 proteins or biologically active portions thereof, as well as nucleic acid fragments suitable as primers or hybridization probes for the detection of TWIK-9-encoding nucleic acids.

In one embodiment, the invention features an isolated nucleic acid molecule that includes the nucleotide sequence set forth in SEQ ID NO:40 or 42. In another embodiment, the invention features an isolated nucleic acid molecule that encodes a polypeptide including the amino acid sequence set forth in SEQ ID NO:41. In another embodiment, the invention features an isolated nucleic acid molecule that includes the nucleotide sequence contained in the plasmid deposited with ATCC® as Accession Number ______.

In still other embodiments, the invention features isolated nucleic acid molecules including nucleotide sequences that are substantially identical (e.g., 60% identical) to the nucleotide sequence set forth as SEQ ID NO:40 or 42. The invention further features isolated nucleic acid molecules including at least 30 contiguous nucleotides of the nucleotide sequence set forth as SEQ ID NO:40 or 42. In another embodiment, the invention features isolated nucleic acid molecules which encode a polypeptide including an amino acid sequence that is substantially identical (e.g., 60% identical) to the amino acid sequence set forth as SEQ ID NO:41. Also featured are nucleic acid molecules which encode allelic variants of the polypeptide having the amino acid sequence set forth as SEQ ID NO:41. In addition to isolated nucleic acid molecules encoding full-length polypeptides, the present invention also features nucleic acid molecules which encode fragments, for example, biologically active or antigenic fragments, of the full-length polypeptides of the present invention (e.g., fragments including at least 10 contiguous amino acid residues of the amino acid sequence of SEQ ID NO:41). In still other embodiments, the invention features nucleic acid molecules that are complementary to, antisense to, or hybridize under stringent conditions to the isolated nucleic acid molecules described herein.

In a related aspect, the invention provides vectors including the isolated nucleic acid molecules described herein (e.g., TWIK-9-encoding nucleic acid molecules). Such vectors can optionally include nucleotide sequences encoding heterologous polypeptides. Also featured are host cells including such vectors (e.g., host cells including vectors suitable for producing TWIK-9 nucleic acid molecules and polypeptides).

In another aspect, the invention features isolated TWIK-9 polypeptides and/or biologically active or antigenic fragments thereof. Exemplary embodiments feature a polypeptide including the amino acid sequence set forth as SEQ ID NO:41, a polypeptide including an amino acid sequence at least 60% identical to the amino acid sequence set forth as SEQ ID NO:41, a polypeptide encoded by a nucleic acid molecule including a nucleotide sequence at least 60% identical to the nucleotide sequence set forth as SEQ ID NO:40 or 42. Also featured are fragments of the full-length polypeptides described herein (e.g., fragments including at least 10 contiguous amino acid residues of the sequence set forth as SEQ ID NO:41) as well as allelic variants of the polypeptide having the amino acid sequence set forth as SEQ ID NO:41.

The TWIK-9 polypeptides and/or biologically active or antigenic fragments thereof, are useful, for example, as reagents or targets in assays applicable to treatment and/or diagnosis of TWIK-9 mediated or potassium channel related disorders. In one embodiment, a TWIK-9 polypeptide or fragment thereof has a TWIK-9 activity. In another embodiment, a TWIK-9 polypeptide or fragment thereof has at least one or more of the following domains: at least one, preferably two, three or four transmembrane domains, at least one, preferably two pore loops (P-loops), and optionally, has a TWIK-9 activity. In a related aspect, the invention features antibodies (e.g., antibodies which specifically bind to any one of the polypeptides, as described herein) as well as fusion polypeptides including all or a fragment of a polypeptide described herein.

The present invention further features methods for detecting TWIK-9 polypeptides and/or TWIK-9 nucleic acid molecules, such methods featuring, for example, a probe, primer or antibody described herein. Also featured are kits for the detection of TWIK-9 polypeptides and/or TWIK-9 nucleic acid molecules. In a related aspect, the invention features methods for identifying compounds which bind to and/or modulate the activity of a TWIK-9 polypeptide or TWIK-9 nucleic acid molecule described herein. Also featured are methods for modulating a TWIK-9 activity.

Other features and advantages of the invention will be apparent from the following detailed description and claims.

### Detailed Description of the Invention

The present invention is based, at least in part, on the discovery of novel molecules, referred to herein as TWIK-9 nucleic acid and protein molecules, which are novel members of the TWIK (for Tandem of P domains in a Weak Inward rectifying K⁺ channel)-like family of potassium channels. These novel molecules are capable of, for example, modulating a potassium channel mediated activity in a cell, e.g., a neuronal cell.

As used herein, a “potassium channel” includes a protein or polypeptide which is involved in receiving, conducting, and transmitting signals in an electrically excitable cell, e.g., a neuronal cell, or a muscle cell (e.g., a cardiac muscle cell). Potassium channels are potassium ion selective, and can determine membrane excitability (the ability of, for example, a neuron to respond to a stimulus and convert it into an impulse). Potassium channels can also influence the resting potential of membranes, wave forms and frequencies of action potentials, and thresholds of excitation. Potassium channels are typically expressed in electrically excitable cells, e.g., neurons, muscle, endocrine, and egg cells, and may form heteromultimeric structures, e.g., composed of pore-forming α and cytoplasmic β subunits. Potassium channels may also be found in non-excitable cells (e.g., spleen cells or prostate cells), where they may play a role in, e.g., signal transduction. Examples of potassium channels include: (1) the voltage-gated potassium channels, (2) the ligand-gated potassium channels, e.g., neurotransmitter-gated potassium channels, and (3) cyclic-nucleotide-gated potassium channels. Voltage-gated and ligand-gated potassium channels are expressed in the brain, e.g., in brainstem monoaminergic and forebrain cholinergic neurons, where they are involved in the release of neurotransmitters, or in the dendrites of hippocampal and neocortical pyramidal cells, where they are involved in the processes of learning and memory formation. For a detailed description of potassium channels, see Kandel E. R.. et al., Principles of Neural Science, second edition, (Elsevier Science Publishing Co., Inc., N.Y. (1985)), the contents of which are incorporated herein by reference. As the TWIK-like proteins of the present invention may modulate potassium channel mediated activities, they may be useful for developing novel diagnostic and therapeutic agents for potassium channel associated disorders.

As used herein, a “potassium channel mediated activity” includes an activity which involves a potassium channel, e.g., a potassium channel in a neuronal cell, or a muscle cell (e.g., a cardiac muscle cell), associated with receiving, conducting, and transmitting signals in, for example, the nervous system. Potassium channel mediated activities include release of neurotransmitters, e.g., dopamine or norepinephrine, from cells, e.g., neuronal cells; modulation of resting potential of membranes, wave forms and frequencies of action potentials, and thresholds of excitation; and modulation of processes such as integration of sub-threshold synaptic responses, the conductance of back-propagating action potentials in, for example, neuronal cells or muscle cells, participation in signal transduction pathways, and participation in nociception.

As used herein, a “potassium channel associated disorder” includes a disorder, disease or condition which is characterized by a misregulation of a potassium channel mediated activity. Potassium channel associated disorders can detrimentally affect conveyance of sensory impulses from the periphery to the brain and/or conductance of motor impulses from the brain to the periphery; integration of reflexes; interpretation of sensory impulses; and emotional, intellectual (e.g., learning and memory), or motor processes.

Examples of potassium channel associated disorders include central nervous system (CNS) disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, movement disorders, progressive supranuclear palsy, epilepsy, AIDS related dementia, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further examples of potassium channel associated disorders include cardiac-related disorders. Cardiovascular system disorders in which the TWIK-9 molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, cerebral ischemia, stroke, coronary artery spasm, and arrhythmia. TWIK-9-mediated or related disorders also include disorders of the musculoskeletal system such as paralysis and muscle weakness, e.g., ataxia, myotonia, and myokymia.

Further disorders in which the TWIK-9 molecules of the invention may be involved are pain disorders. Pain disorders include those that affect pain signaling mechanisms. As used herein, the term “pain signaling mechanisms” includes the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter sensory neurons. These sensory neurons transmit the information to the central nervous system, evoking a perception of pain or discomfort and initiating appropriate protective reflexes. The TWIK-9 molecules of the present invention may be present on these sensory neurons and, thus, may be involved in detecting these noxious chemical, mechanical, or thermal stimuli and transducing this information into membrane depolarization events. Thus, the TWIK-9 molecules by participating in pain signaling mechanisms, may modulate pain elicitation and act as targets for developing novel diagnostic targets and therapeutic agents to control pain. Examples of pain disorders include headache (e.g., tension headache or migraine), back pain, cancer pain, arthritis pain, or neurogenic pain.

Potassium channel disorders also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The TWIK-9 molecules of the present invention are involved in signal transduction mechanisms, which are known to be involved in cellular growth, differentiation, and migration processes. Thus, the TWIK-9 molecules may modulate cellular growth, differentiation, or migration, and may play a role in disorders characterized by aberrantly regulated growth, differentiation, or migration. Such disorders include cancer, e.g., carcinoma, sarcoma, lymphoma or leukemia, examples of which include, but are not limited to, breast, endometrial, ovarian, uterine, hepatic, gastrointestinal, prostate, colorectal, and lung cancer, melanoma, neurofibromatosis, adenomatous polyposis of the colon, Wilms' tumor, nephroblastoma, teratoma, rhabdomyosarcoma; tumor invasion, angiogenesis and metastasis; skeletal dysplasia; neuronal deficiencies resulting from impaired neural induction and patterning; and hematopoietic and/or myeloproliferative disorders.

TWIK-9-associated or related disorders also include disorders of tissues in which TWIK-9 protein is predominantly expressed, e.g., dorsal root ganglia, cerebellum, testes, mammary gland, fetal brain and fetal thymus.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin or alternatively, can contain homologues of non-human origin, e.g., rat or mouse proteins. Members of a family can also have common functional characteristics.

For example, the family of TWIK-9 proteins comprises at least one “transmembrane domain” and preferably two, three, or four transmembrane domains. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al. (1996) Annual Rev. Neurosci. 19: 235-263, the contents of which are incorporated herein by reference. Amino acid residues 9-26, 108-125, 159-178 and 219-243 of the TWIK-9 protein are predicted to comprise transmembrane domains.

In another embodiment, a TWIK-9 molecule of the present invention is identified based on the presence of at least one, preferably two pore loops (P-loops). As used herein, the term “pore loop” or “P-loop” includes an amino acid sequence of about 15-45 amino acid residues in length, preferably about 15-35 amino acid residues in length, and most preferably about 15-25 amino acid residues in length, which is involved in lining the potassium channel pore. A P-loop is typically found between transmembrane domains of potassium channels and is believed to be a major determinant of ion selectivity in potassium channels. Preferably, P-loops contain a G-[HYDROPHOBIC AMINO ACID]-G sequence, e.g., a GYG, GLG, or GFG sequence. P-loops are described in, for example, Warmke et al. (1991) Science 252:1560-1562; Zagotta W. N. et al., (1996) Annual Rev. Neurosci. 19:235-63 (Pongs, O. (1993) J. Membr. Biol., 136, 1-8; Heginbotham et al. (1994) Biophys. J. 66,1061-1067; Mackinnon, R. (1995) Neuron, and 14, 889-892; Pascual et al., (1995) Neuron., 14, 1055-1063), the contents of which are incorporated herein by reference. Amino acid residues 80-97 and 190-208 of the human TWIK-9 protein comprise a P-loop.

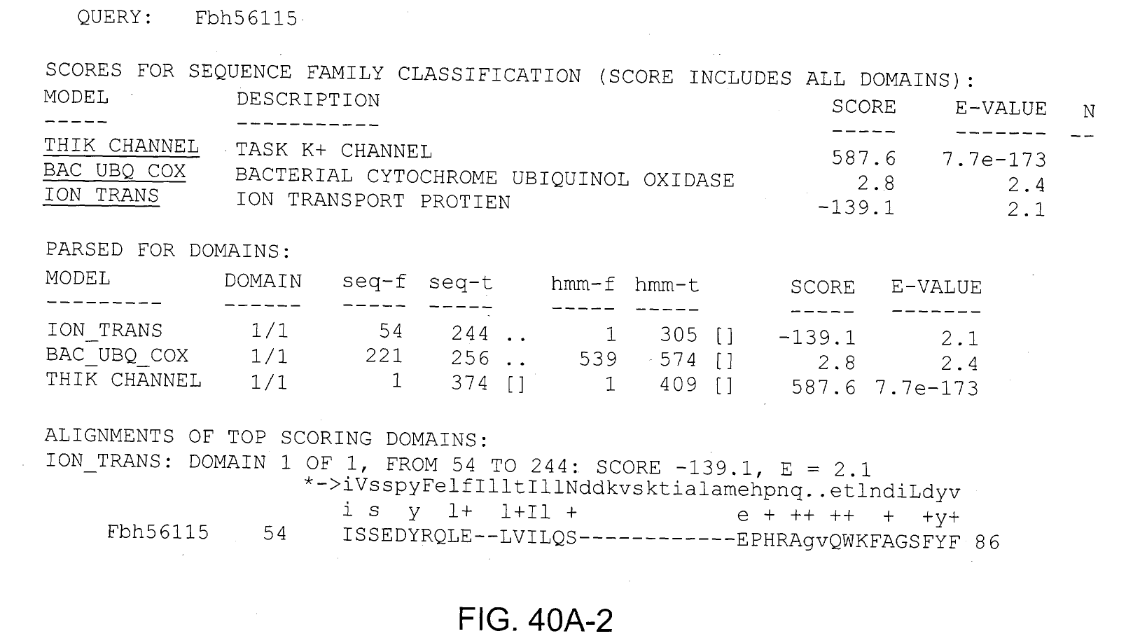

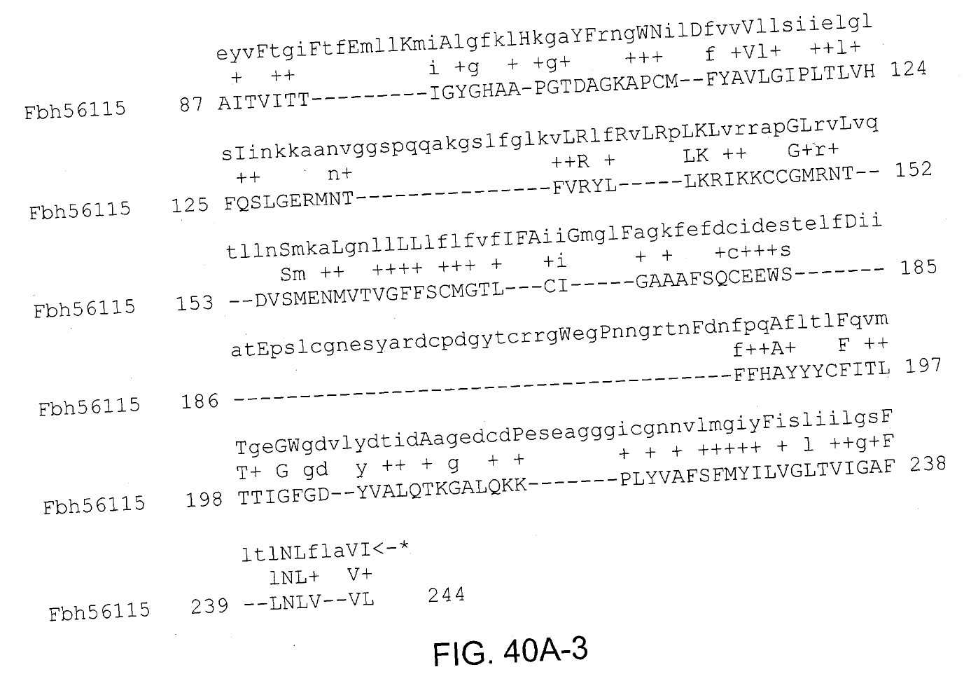

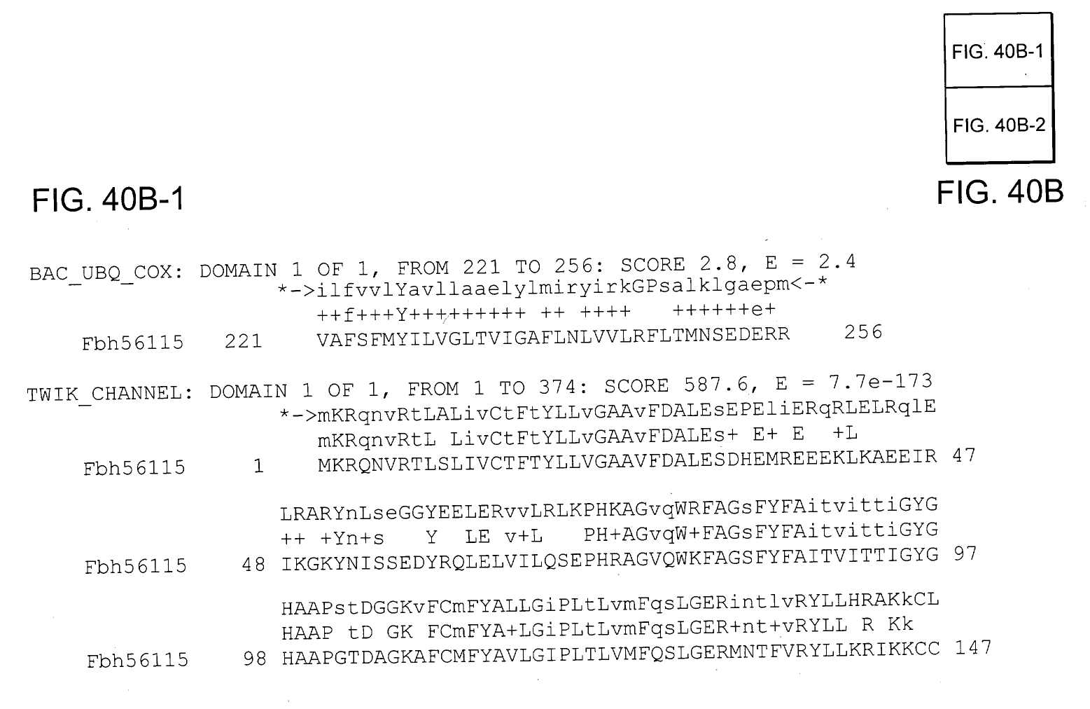

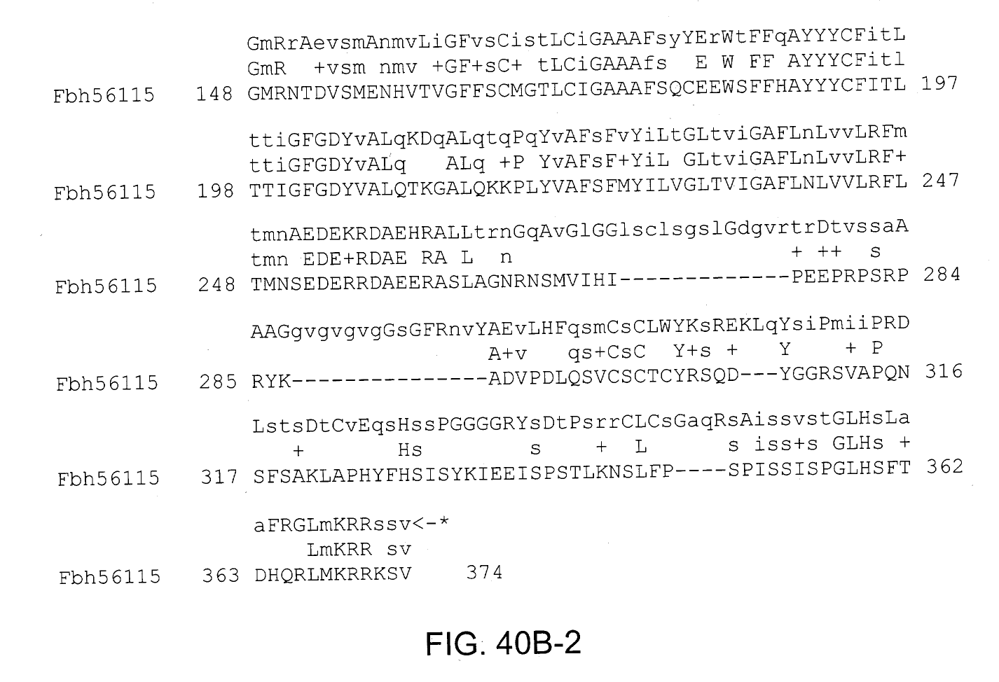

In another embodiment, a TWIK-9 molecule of the present invention is identified based on a high scoring match with a TWIK channel Hidden Markov Model (i.e., a score of greater than 100, greater than 200, greater than 300, greater than 400 or greater than 500). To identify a high scoring match between a protein of interest and a TWIK channel HMM, the amino acid sequence of the protein is searched against a database of known HMMs (e.g., the Washington University HMM database). The TWIK channel (HMM) has been assigned the PFAM Accession PF02034 (http://genome.wustl.edu/Pfam/html). A search was performed against the HMM database resulting in a high scoring match between SEQ ID NO:41 and the TWIK channel HMM, e.g., a match having a score of greater than 587. The results of the search are set forth in FIG. 40.

In a preferred embodiment, the TWIK-9 molecules of the invention include at least one and, preferably, two, three, or four transmembrane domains, and at least one, preferably two P loops. In another preferred embodiment, the TWIK-9 molecules of the invention include at least one and, preferably, two, three, or four transmembrane domains, and at least one, preferably two P loops, and have a high scoring match with a TWIK channel HMM.

A description of the Pfam database can be found in Sonhammer et al. (1997) Proteins 28:405-420, and a detailed description of HMMs can be found, for example, in Gribskov et al.(1990) Meth. Enzymol. 183:146-159; Gribskov et al. (1987) Proc. Natl. Acad. Sci. USA 84:4355-4358; Krogh et al.(1994) J. Mol. Biol. 235:1501-1531; and Stultz et al.(1993) Protein Sci. 2:305-314, the contents of which are incorporated herein by reference.

The signature patterns or consensus patterns described herein are described according to the following designation: all amino acids are indicated according to their universal single letter designation; “x” designates any amino acid; x(n) designates n number of amino acids, e.g., x (2) designates any two amino acids, e.g., x (1-3) designates any of one to three amino acids; and, amino acids in brackets indicates any one of the amino acids within the brackets, e.g., [RK] indicates any of one of either R (arginine) or K (lysine).

Isolated proteins of the present invention, preferably TWIK-9 proteins, have an amino acid sequence sufficiently homologous to the amino acid sequence of SEQ ID NO:41, or are encoded by a nucleotide sequence sufficiently homologous to SEQ ID NO:40 or 42. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains having at least 50%, 55%, 60%, 65%, 70%, 75%, 80%, 85%, 90%, 91%, 92%, 93%, 94%, 95%, 96%, 97%, 98%, 99% or more homology or identity across the amino acid sequences of the domains and contain at least one and preferably two structural domains or motifs, are defined herein as sufficiently homologous. Furthermore, amino acid or nucleotide sequences which share at least 50%, 55%, 60%, 65%, 70%, 75%, 80%, 85%, 90%, 91%, 92%, 93%, 94%, 95%, 96%, 97%, 98%, 99% or more homology or identity and share a common functional activity are defined herein as sufficiently homologous.

In a preferred embodiment, a TWIK-9 protein includes at least one, preferably two, three, or four transmembrane domains, and at least one, preferably two pore loops (P-loops), and has an amino acid sequence at least about 50%, 55%, 60%, 65%, 70%, 75%, 80%, 85%, 85%, 90%, 95%, 96%, 97%, 98%, 99% or more homologous or identical to the amino acid sequence of SEQ ID NO:41, or the amino acid sequence encoded by the DNA insert of the plasmid deposited with ATCC as Accession Number ______. In yet another preferred embodiment, a TWIK-9 protein includes at least one, preferably two, three, or four transmembrane domains, and at least one, preferably two pore loops (P-loops), and is encoded by a nucleic acid molecule having a nucleotide sequence which hybridizes under stringent hybridization conditions to a complement of a nucleic acid molecule comprising the nucleotide sequence of SEQ ID NO:40 or 42. In another preferred embodiment, a TWIK-9 protein includes at least one, preferably two, three, or four transmembrane domains, and at least one, preferably two pore loops (P-loops), and has a TWIK-9 activity.

As used interchangeably herein, a “TWIK-9 activity”, “biological activity of TWIK-9” or “functional activity of TWIK-9”, includes an activity exerted or mediated by a TWIK-9 protein, polypeptide or nucleic acid molecule on a TWIK-9 responsive cell or tissue, or on a TWIK-9 substrate, as determined in vivo or in vitro, according to standard techniques. In one embodiment, a TWIK-9 activity is a direct activity, such as an association with a TWIK-9 target molecule. As used herein, a “target molecule” or “binding partner” is a molecule with which a TWIK-9 protein binds or interacts in nature, such that TWIK-9-mediated function is achieved. A TWIK-9 target molecule can be a non-TWIK-9 molecule or a TWIK-9 protein or polypeptide of the present invention. In an exemplary embodiment, a TWIK-9 target molecule is a TWIK-9 ligand, e.g., a potassium channel pore forming subunit or a potassium channel ligand. A TWIK-9 activity can also be an indirect activity, such as a cellular signaling activity mediated by interaction of the TWIK-9 protein with a TWIK-9 ligand or substrate. The biological activities of TWIK-9 are described herein.

For example, the TWIK-9 proteins of the present invention can have one or more of the following activities: (1) interacting with a non-TWIK protein molecule; (2) activating a TWIK-dependent signal transduction pathway; (3) modulating the release of neurotransmitters; (4) modulating membrane excitability; (5) influencing the resting potential of membranes, wave forms and frequencies of action potentials, and thresholds of excitation, (6) modulating processes which underlie learning and memory, such as integration of sub-threshold synaptic responses and the conductance of back-propagating action potentials, and (7) mediating nociception.

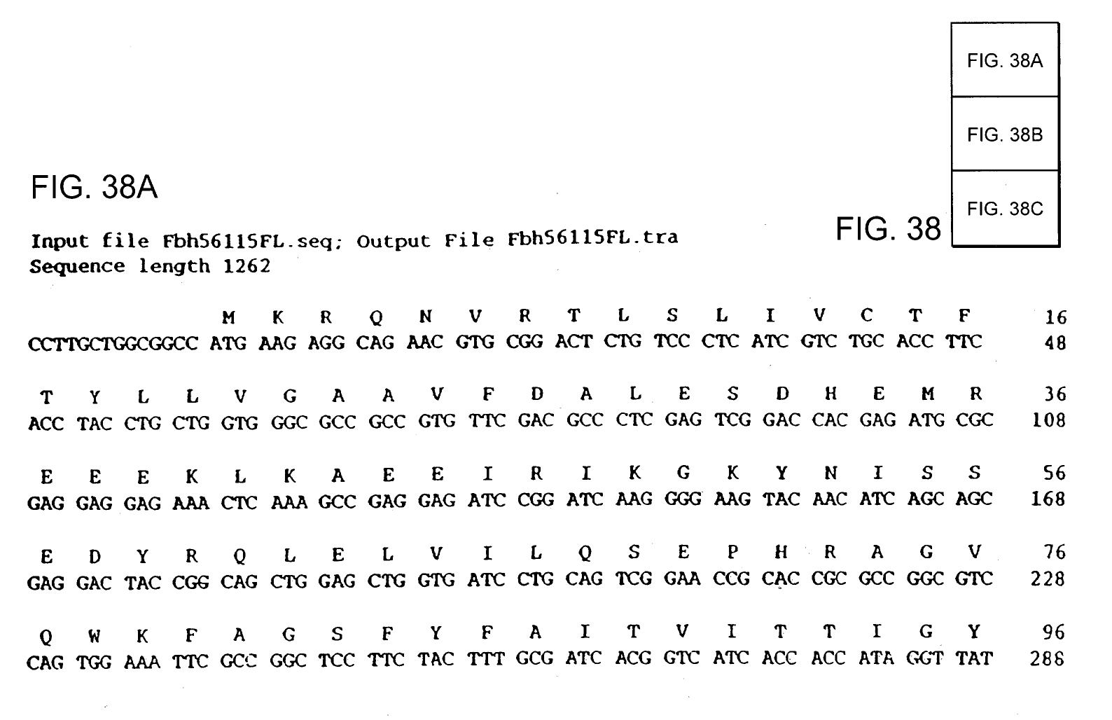

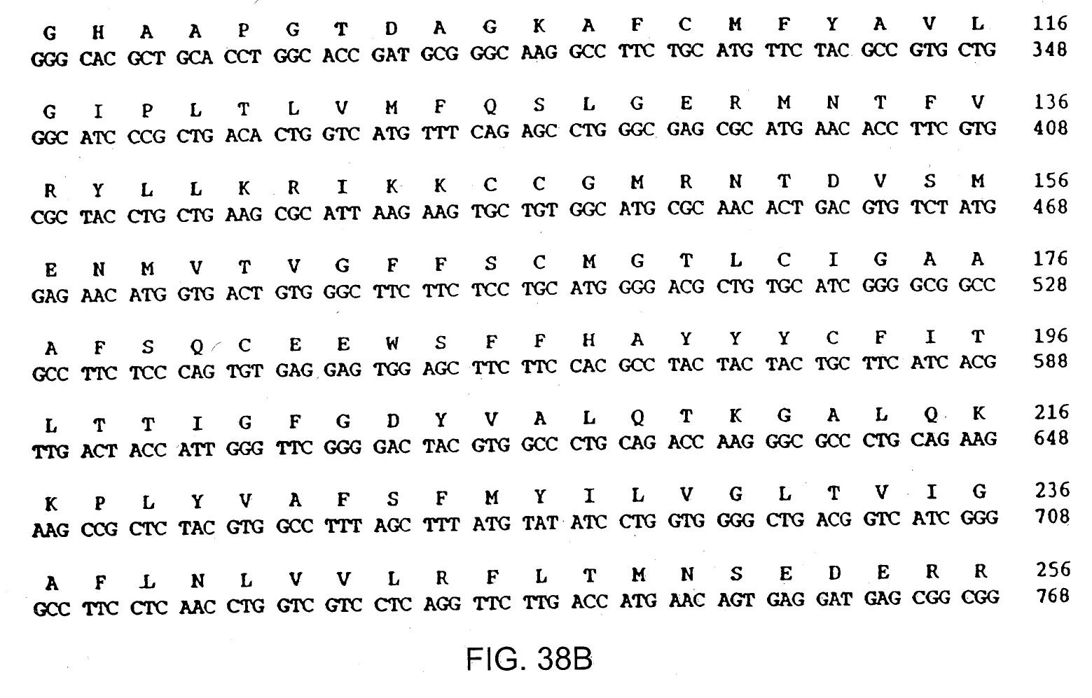

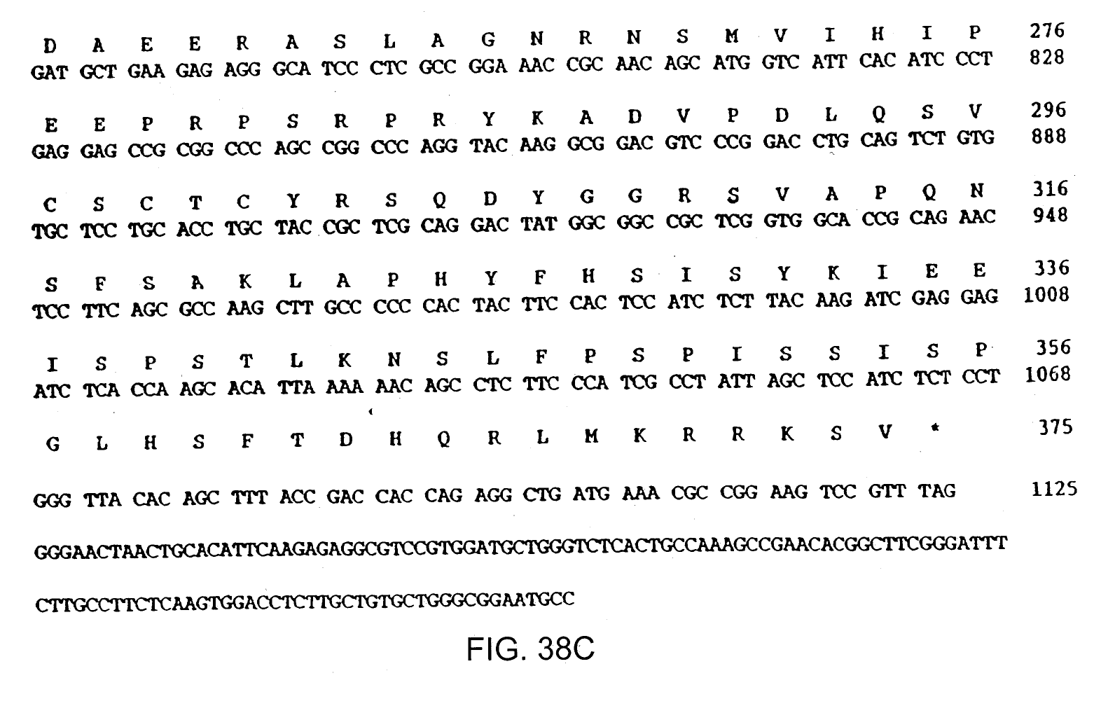

The nucleotide sequence of the isolated human TWIK-9 cDNA and the predicted amino acid sequence encoded by the TWIK-9 cDNA are shown in FIG. 38 and in SEQ ID NOs:40 and 41, respectively. A plasmid containing the human TWIK-9 cDNA was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit were made merely as a convenience for those of skill in the art and is not an admission that a deposit is required under 35 U.S.C. §112.

The human TWIK-9 gene, which is approximately 1262 nucleotides in length, encodes a protein having a molecular weight of approximately 41 kD and which is approximately 374 amino acid residues in length.

Various aspects of the invention are described in further detail in the following subsections:

I. Isolated Nucleic Acid Molecules

One aspect of the invention pertains to isolated nucleic acid molecules that encode TWIK-9 proteins or biologically active portions thereof, as well as nucleic acid fragments sufficient for use as hybridization probes to identify TWIK-9-encoding nucleic acid molecules (e.g, TWIK-9 mRNA) and fragments for use as PCR primers for the amplification or mutation of TWIK-9 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TWIK-9 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:40 or 42, or the nucleotide sequence of the DNA insert of the plasmid deposited with ATCC as Accession Number ______, or a portion thereof, can be isolated using standard molecular biology techniques and the sequence information provided herein. Using all or a portion of the nucleic acid sequence of SEQ ID NO:40 or 42, or the nucleotide sequence of the DNA insert of the plasmid deposited with ATCC as Accession Number ______, as hybridization probes, TWIK-9 nucleic acid molecules can be isolated using standard hybridization and cloning techniques (e.g., as described in Sambrook, J., Fritsh, E. F., and Maniatis, T. Molecular Cloning: A Laboratory Manual. 2^{nd}, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:40 or 42, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:40 or 42, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to TWIK-9 nucleotide sequences can be prepared by standard synthetic techniques, e.g., using an automated DNA synthesizer.

In one embodiment, an isolated nucleic acid molecule of the invention comprises the nucleotide sequence shown in SEQ ID NO:40 or 42. This cDNA may comprise sequences encoding the human TWIK-9 protein (e.g., the “coding region”, from nucleotides 15-1139), as well as 5′ untranslated sequence (nucleotides 1-14) and 3′ untranslated sequences (nucleotides 1140-1262) of SEQ ID NO:40. Alternatively, the nucleic acid molecule can comprise only the coding region of SEQ ID NO:40 (e.g., nucleotides 15-1139, corresponding to SEQ ID NO:42). Accordingly, in another embodiment, an isolated nucleic acid molecule of the invention comprises SEQ ID NO:42 and nucleotides 1-14 of SEQ ID NO:40. In yet another embodiment, the isolated nucleic acid molecule comprises SEQ ID NO:42 and nucleotides 1140-1262 of SEQ ID NO:40. In yet another embodiment, the nucleic acid molecule consists of the nucleotide sequence set forth as SEQ ID NO:40 or 42.

In still another embodiment, an isolated nucleic acid molecule of the invention comprises a nucleic acid molecule which is a complement of the nucleotide sequence shown in SEQ ID NO:40 or 42, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:40 or 42, or the nucleotide sequence of the DNA insert of the plasmid deposited with ATCC as Accession Number ______, is one which is sufficiently complementary to the nucleotide sequence shown in SEQ ID NO:40 or 42, or the nucleotide sequence of the DNA insert of the plasmid deposited with ATCC as Accession Number ______, such that it can hybridize to the nucleotide sequence shown in SEQ ID NO:40 or 42, or the nucleotide sequence of the DNA insert of the plasmid deposited with ATCC as Accession Number ______, thereby forming a stable duplex.

In still another embodiment, an isolated nucleic acid molecule of the present invention comprises a nucleotide sequence which is at least about 50%, 55%, 60%, 65%, 70%, 75%, 80%, 85%, 90%, 91%, 92%, 93%, 94%, 95%, 96%, 97%, 98%, 99% or more identical to the nucleotide sequence shown in SEQ ID NO:40 or 42 (e.g., to the entire length of the nucleotide sequence), or to the nucleotide sequence (e.g., the entire length of the nucleotide sequence) of the DNA insert of the plasmid deposited with ATCC as Accession Number ______, or a portion or complement of any of these nucleotide sequences. In one embodiment, a nucleic acid molecule of the present invention comprises a nucleotide sequence which is at least (or no greater than) 50-100, 100-200, 200-300, 305, 305-400, 400-500, 500-600, 600-700, 700-800, 800-900, 900-1000, 1000-1100, 1000-1200 or more nucleotides in length and hybridizes under stringent hybridization conditions to a complement of a nucleic acid molecule of SEQ ID NO:40 or 42, or the nucleotide sequence of the DNA insert of the plasmid deposited with ATCC as Accession Number ______.

Moreover, the nucleic acid molecule of the invention can comprise only a portion of the nucleic acid sequence of SEQ ID NO:40 or 42, or the nucleotide sequence of the DNA insert of the plasmid deposited with ATCC as Accession Number ______, for example, a fragment which can be used as a probe or primer or a fragment encoding a portion of a TWIK-9 protein, e.g., a biologically active portion of a TWIK-9 protein. The nucleotide sequence determined from the cloning of the TWIK-9 gene allows for the generation of probes and primers designed for use in identifying and/or cloning other TWIK-9 family members, as well as TWIK-9 homologues from other species. The probe/primer (e.g., oligonucleotide) typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:40 or 42, or the nucleotide sequence of the DNA insert of the plasmid deposited with ATCC as Accession Number ______, of an anti-sense sequence of SEQ ID NO:40 or 42, or the nucleotide sequence of the DNA insert of the plasmid deposited with ATCC as Accession Number _______, or of a naturally occurring allelic variant or mutant of SEQ ID NO:40 or 42, or the nucleotide sequence of the DNA insert of the plasmid deposited with ATCC as Accession Number ______.

Exemplary probes or primers are at least (or no greater than) 12 or 15, 20 or 25, 30, 35, 40, 45, 50, 55, 60, 65, 70, 75 or more nucleotides in length and/or comprise consecutive nucleotides of an isolated nucleic acid molecule described herein. Also included within the scope of the present invention are probes or primers comprising contiguous or consecutive nucleotides of an isolated nucleic acid molecule described herein, but for the difference of 1, 2, 3, 4, 5, 6, 7, 8, 9 or 10 bases within the probe or primer sequence. Probes based on the TWIK-9 nucleotide sequences can be used to detect (e.g., specifically detect) transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of a TWIK-9 sequence, e.g., a domain, region, site or other sequence described herein. The primers should be at least 5, 10, or 50 base pairs in length and less than 100, or less than 200, base pairs in length. The primers should be identical, or differ by no greater than 1, 2, 3, 4, 5, 6, 7, 8, 9 or 10 bases when compared to a sequence disclosed herein or to the sequence of a naturally occurring variant. Such probes can be used as a part of a diagnostic test kit for identifying cells or tissue which misexpress a TWIK-9 protein, such as by measuring a level of a TWIK-9-encoding nucleic acid in a sample of cells from a subject, e.g., detecting TWIK-9 mRNA levels or determining whether a genomic TWIK-9 gene has been mutated or deleted.

A nucleic acid fragment encoding a “biologically active portion of a TWIK-9 protein” can be prepared by isolating a portion of the nucleotide sequence of SEQ ID NO:40 or 42, or the nucleotide sequence of the DNA insert of the plasmid deposited with ATCC as Accession Number ______, which encodes a polypeptide having a TWIK-9 biological activity (the biological activities of the TWIK-9 proteins are described herein), expressing the encoded portion of the TWIK-9 protein (e.g., by recombinant expression in vitro) and assessing the activity of the encoded portion of the TWIK-9 protein. In an exemplary embodiment, the nucleic acid molecule is at least 50-100, 100-200, 200-300, 305, 305-400, 400-500, 500-600, 600-700, 700-800, 800-900, 900-1000, 1000-1100, 1000-1200 or more nucleotides in length and encodes a protein having a TWIK-9 activity (as described herein).

The invention further encompasses nucleic acid molecules that differ from the nucleotide sequence shown in SEQ ID NO:40 or 42, or the nucleotide sequence of the DNA insert of the plasmid deposited with ATCC as Accession Number ______, due to degeneracy of the genetic code and thus encode the same TWIK-9 proteins as those encoded by the nucleotide sequence shown in SEQ ID NO:40 or 42, or the nucleotide sequence of the DNA insert of the plasmid deposited with ATCC as Accession Number ______. In another embodiment, an isolated nucleic acid molecule of the invention has a nucleotide sequence encoding a protein having an amino acid sequence which differs by at least 1, but no greater than 5, 10, 20, 50 or 100 amino acid residues from the amino acid sequence shown in SEQ ID NO:41, or the amino acid sequence encoded by the DNA insert of the plasmid deposited with the ATCC as Accession Number ______. In yet another embodiment, the nucleic acid molecule encodes the amino acid sequence of human TWIK-9. If an alignment is needed for this comparison, the sequences should be aligned for maximum homology.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

Allelic variants result, for example, from DNA sequence polymorphisms within a population (e.g., the human population) that lead to changes in the amino acid sequences of the TWIK-9 proteins. Such genetic polymorphism in the TWIK-9 genes may exist among individuals within a population due to natural allelic variation. As used herein, the terms “gene” and “recombinant gene” refer to nucleic acid molecules which include an open reading frame encoding a TWIK-9 protein, preferably a mammalian TWIK-9 protein, and can further include non-coding regulatory sequences, and introns.

Accordingly, in one embodiment, the invention features isolated nucleic acid molecules which encode a naturally occurring allelic variant of a polypeptide comprising the amino acid sequence of SEQ ID NO:41, or an amino acid sequence encoded by the DNA insert of the plasmid deposited with ATCC as Accession Number ______, wherein the nucleic acid molecule hybridizes to a complement of a nucleic acid molecule comprising SEQ ID NO:40 or 42, for example, under stringent hybridization conditions.

Allelic variants of TWIK-9, e.g., human TWIK-9, include both functional and non-functional TWIK-9 proteins. Functional allelic variants are naturally occurring amino acid sequence variants of the TWIK-9 protein that maintain the ability, for example, to bind a TWIK-9 ligand or substrate, and/or modulate pain signaling mechanisms, membrane excitability or neurotransmitter release. Functional allelic variants will typically contain only conservative substitution of one or more amino acids of SEQ ID NO:41, or substitution, deletion or insertion of non-critical residues in non-critical regions of the protein.

Non-functional allelic variants are naturally occurring amino acid sequence variants of the TWIK-9 protein, e.g., human TWIK-9, that do not have the ability to either bind or interact with a TWIK-9 ligand or substrate, and/or modulate any of the TWIK-9 activities described herein. Non-functional allelic variants will typically contain a non-conservative substitution, a deletion, or insertion, or premature truncation of the amino acid sequence of SEQ ID NO:41, or a substitution, insertion, or deletion in critical residues or critical regions of the protein.

The present invention further provides non-human orthologues (e.g., non-human orthologues of the human TWIK-9 protein). Orthologues of the human TWIK-9 protein are proteins that are isolated from non-human organisms and possess, for example, the same TWIK-9 ligand or substrate binding, and/or modulation of pain signaling mechanisms, membrane excitability or neurotransmitter release activities of the human TWIK-9 protein. Orthologues of the human TWIK-9 protein can readily be identified as comprising an amino acid sequence that is substantially homologous to SEQ ID NO:41.

Moreover, nucleic acid molecules encoding other TWIK-9 family members and, thus, which have a nucleotide sequence which differs from the TWIK-9 sequences of SEQ ID NO:40 or 42, or the nucleotide sequence of the DNA insert of the plasmid deposited with ATCC as Accession Number ______ are intended to be within the scope of the invention. For example, another TWIK-9 cDNA can be identified based on the nucleotide sequence of human TWIK-9. Moreover, nucleic acid molecules encoding TWIK-9 proteins from different species, and which, thus, have a nucleotide sequence which differs from the TWIK-9 sequences of SEQ ID NO:40 or 42, or the nucleotide sequence of the DNA insert of the plasmid deposited with ATCC as Accession Number ______ are intended to be within the scope of the invention. For example, a mouse or monkey TWIK-9 cDNA can be identified based on the nucleotide sequence of a human TWIK-9.

Nucleic acid molecules corresponding to natural allelic variants and homologues of the TWIK-9 cDNAs of the invention can be isolated based on their homology to the TWIK-9 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the TWIK-9 cDNAs of the invention can further be isolated by mapping to the same chromosome or locus as the TWIK-9 gene.

Orthologues, homologues and allelic variants can be identified using methods known in the art (e.g., by hybridization to an isolated nucleic acid molecule of the present invention, for example, under stringent hybridization conditions). In one embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:40 or 42, or the nucleotide sequence of the DNA insert of the plasmid deposited with ATCC as Accession Number ______. In other embodiment, the nucleic acid is at least 50-100, 100-200, 200-300, 305, 305-400, 400-500, 500-600, 600-700, 700-800, 800-900, 900-1000, 1000-1100, 1000-1200 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4×sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

Preferably, an isolated nucleic acid molecule of the invention that hybridizes under stringent conditions to the sequence of SEQ ID NO:40 or 42 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the TWIK-9 sequences that may exist in the population, the skilled artisan will further appreciate that changes can be introduced by mutation into the nucleotide sequences of SEQ ID NO:40 or 42, or the nucleotide sequence of the DNA insert of the plasmid deposited with ATCC as Accession Number ______, thereby leading to changes in the amino acid sequence of the encoded TWIK-9 proteins, without altering the functional ability of the TWIK-9 proteins. For example, nucleotide substitutions leading to amino acid substitutions at “non-essential” amino acid residues can be made in the sequence of SEQ ID NO:40 or 42, or the nucleotide sequence of the DNA insert of the plasmid deposited with ATCC as Accession Number ______. A “non-essential” amino acid residue is a residue that can be altered from the wild-type sequence of TWIK-9 (e.g., the sequence of SEQ ID NO:41) without altering the biological activity, whereas an “essential” amino acid residue is required for biological activity. For example, amino acid residues that are conserved among the TWIK-9 proteins of the present invention, e.g., those present in a transmembrane domain or P loop, are predicted to be particularly unamenable to alteration. Furthermore, additional amino acid residues that are conserved between the TWIK-9 proteins of the present invention and other members of the TWIK family are not likely to be amenable to alteration.

Accordingly, another aspect of the invention pertains to nucleic acid molecules encoding TWIK-9 proteins that contain changes in amino acid residues that are not essential for activity. Such TWIK-9 proteins differ in amino acid sequence from SEQ ID NO:41, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 91%, 92% 93%, 94%, 95%, 96%, 97%, 98% 99% or more homologous to SEQ ID NO:41, e.g., to the entire length of SEQ ID NO:41.

An isolated nucleic acid molecule encoding a TWIK-9 protein homologous to the protein of SEQ ID NO:41 can be created by introducing one or more nucleotide substitutions, additions or deletions into the nucleotide sequence of SEQ ID NO:40 or 42, or the nucleotide sequence of the DNA insert of the plasmid deposited with ATCC as Accession Number ______, such that one or more amino acid substitutions, additions or deletions are introduced into the encoded protein. Mutations can be introduced into SEQ ID NO:40 or 42, or the nucleotide sequence of the DNA insert of the plasmid deposited with ATCC as Accession Number ______ by standard techniques, such as site-directed mutagenesis and PCR-mediated mutagenesis.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a TWIK-9 protein is preferably replaced with another amino acid residue from the same side chain family. Alternatively, in another embodiment, mutations can be introduced randomly along all or part of a TWIK-9 coding sequence, such as by saturation mutagenesis, and the resultant mutants can be screened for TWIK-9 biological activity to identify mutants that retain activity. Following mutagenesis of SEQ ID NO:40 or 42, or the nucleotide sequence of the DNA insert of the plasmid deposited with ATCC as Accession Number ______, the encoded protein can be expressed recombinantly and the activity of the protein can be determined.

In a preferred embodiment, a mutant TWIK-9 protein can be assayed for the ability to: (1) interact with a non-TWIK protein molecule; (2) activate a TWIK-dependent signal transduction pathway; (3) modulate the release of neurotransmitters; (4) modulating membrane excitability; (5) influence the resting potential of membranes, wave forms and frequencies of action potentials, and thresholds of excitation, (6) modulate processes which underlie learning and memory, such as integration of sub-threshold synaptic responses and the conductance of back-propagating action potentials, and (7) mediate nociception.

In addition to the nucleic acid molecules encoding TWIK-9 proteins described above, another aspect of the invention pertains to isolated nucleic acid molecules which are antisense thereto. In an exemplary embodiment, the invention provides an isolated nucleic acid molecule which is antisense to a TWIK-9 nucleic acid molecule (e.g., is antisense to the coding strand of a TWIK-9 nucleic acid molecule). An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TWIK-9 coding strand, or to only a portion thereof. In one embodiment, an antisense nucleic acid molecule is antisense to “coding region sequences” of the coding strand of a nucleotide sequence encoding TWIK-9. The term “coding region sequences” refers to the region of the nucleotide sequence comprising codons which are translated into amino acid residues (e.g., the coding region sequences of human TWIK-9 corresponding to SEQ ID NO:42). In another embodiment, the antisense nucleic acid molecule is antisense to a “noncoding region” of the coding strand of a nucleotide sequence encoding TWIK-9. The term “noncoding region” refers to 5′ and/or 3′ sequences which flank the coding region sequences that are not translated into amino acids (also referred to as 5′ and 3′ untranslated regions).

Given the coding strand sequences encoding TWIK-9 disclosed herein (e.g., SEQ ID NO:42), antisense nucleic acids of the invention can be designed according to the rules of Watson and Crick base pairing. The antisense nucleic acid molecule can be complementary to coding region sequences of TWIK-9 mRNA, but more preferably is an oligonucleotide which is antisense to only a portion of the TWIK-9 mRNA. An antisense oligonucleotide can be, for example, about 5, 10, 15, 20, 25, 30, 35, 40, 45, 50, 55, 60, 65, 70, 75, 80, or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a TWIK-9 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave TWIK-9 mRNA transcripts to thereby inhibit translation of TWIK-9 mRNA. A ribozyme having specificity for a TWIK-9-encoding nucleic acid can be designed based upon the nucleotide sequence of a TWIK-9 cDNA disclosed herein (i.e., SEQ ID NO:40 or 42, or the nucleotide sequence of the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in a TWIK-9-encoding mRNA. See, e.g., Cech et al. U.S. Pat. No. 4,987,071; and Cech et al. U.S. Pat. No. 5,116,742. Alternatively, TWIK-9 mRNA can be used to select a catalytic RNA having a specific ribonuclease activity from a pool of RNA molecules. See, e.g., Bartel, D. and Szostak, J. W. (1993) Science 261:1411-1418.

Alternatively, TWIK-9 gene expression can be inhibited by targeting nucleotide sequences complementary to the regulatory region of the TWIK-9 (e.g., the TWIK-9 promoter and/or enhancers; e.g., nucleotides 1-14 of SEQ ID NO:40) to form triple helical structures that prevent transcription of the TWIK-9 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the TWIK-9 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. et al. (1996) Bioorganic & Medicinal Chemistry 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, B. et al. (1996) supra; Perry-O'Keefe et al. (1996) Proc. Natl. Acad. Sci. USA 93:14670-675.

PNAs of TWIK-9 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of TWIK-9 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup, B. et al. (1996) supra)); or as probes or primers for DNA sequencing or hybridization (Hyrup, B. et al. (1996) supra; Perry-O'Keefe et al. (1996) supra).

In another embodiment, PNAs of TWIK-9 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of TWIK-9 nucleic acid molecules can be generated which may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, B. et al. (1996) supra). The synthesis of PNA-DNA chimeras can be performed as described in Hyrup, B. et al. (1996) supra and Finn, P. J. et al. (1996) Nucleic Acids Res. 24 (17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17:5973-88). PNA monomers are then coupled in a stepwise manner to produce a chimeric molecule with a 5′ PNA segment and a 3′ DNA segment (Finn, P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous TWIK-9 gene within a cell line or microorganism may be modified by inserting a heterologous DNA regulatory element into the genome of a stable cell line or cloned microorganism such that the inserted regulatory element is operatively linked with the endogenous TWIK-9 gene. For example, an endogenous TWIK-9 gene which is normally “transcriptionally silent”, i.e., a TWIK-9 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous TWIK-9 gene may be activated by insertion of a promiscuous regulatory element that works across cell types.

A heterologous regulatory element may be inserted into a stable cell line or cloned microorganism, such that it is operatively linked with an endogenous TWIK-9 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated TWIK-9 Proteins and Anti-TWIK-9 Antibodies

One aspect of the invention pertains to isolated or recombinant TWIK-9 proteins and polypeptides, and biologically active portions thereof, as well as polypeptide fragments suitable for use as immunogens to raise anti-TWIK-9 antibodies. In one embodiment, native TWIK-9 proteins can be isolated from cells or tissue sources by an appropriate purification scheme using standard protein purification techniques. In another embodiment, TWIK-9 proteins are produced by recombinant DNA techniques. Alternative to recombinant expression, a TWIK-9 protein or polypeptide can be synthesized chemically using standard peptide synthesis techniques.

An “isolated” or “purified” protein or biologically active portion thereof is substantially free of cellular material or other contaminating proteins from the cell or tissue source from which the TWIK-9 protein is derived, or substantially free from chemical precursors or other chemicals when chemically synthesized. The language “substantially free of cellular material” includes preparations of TWIK-9 protein in which the protein is separated from cellular components of the cells from which it is isolated or recombinantly produced. In one embodiment, the language “substantially free of cellular material” includes preparations of TWIK-9 protein having less than about 30% (by dry weight) of non-TWIK-9 protein (also referred to herein as a “contaminating protein”), more preferably less than about 20% of non-TWIK-9 protein, still more preferably less than about 10% of non-TWIK-9 protein, and most preferably less than about 5% non-TWIK-9 protein. When the TWIK-9 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of TWIK-9 protein in which the protein is separated from chemical precursors or other chemicals which are involved in the synthesis of the protein. In one embodiment, the language “substantially free of chemical precursors or other chemicals” includes preparations of TWIK-9 protein having less than about 30% (by dry weight) of chemical precursors or non-TWIK-9 chemicals, more preferably less than about 20% chemical precursors or non-TWIK-9 chemicals, still more preferably less than about 10% chemical precursors or non-TWIK-9 chemicals, and most preferably less than about 5% chemical precursors or non-TWIK-9 chemicals.

As used herein, a “biologically active portion” of a TWIK-9 protein includes a fragment of a TWIK-9 protein which participates in an interaction between a TWIK-9 molecule and a non-TWIK-9 molecule (e.g., a TWIK-9 ligand or substrate). Biologically active portions of a TWIK-9 protein include peptides comprising amino acid sequences sufficiently homologous to or derived from the TWIK-9 amino acid sequences, e.g., the amino acid sequences shown in SEQ ID NO:41, which include sufficient amino acid residues to exhibit at least one activity of a TWIK-9 protein. Typically, biologically active portions comprise a domain or motif with at least one activity of the TWIK-9 protein, e.g., modulation of pain signaling mechanisms, membrane excitability or neurotransmitter release. A biologically active portion of a TWIK-9 protein can be a polypeptide which is, for example, 10, 25, 50, 75, 100, 125, 150, 175, 200, 225, 250, 275, 300 or more amino acids in length. Biologically active portions of a TWIK-9 protein can be used as targets for developing agents which modulate a TWIK-9 mediated activity, e.g., modulation of pain signaling mechanisms, membrane excitability or neurotransmitter release activities.

In one embodiment, a biologically active portion of a TWIK-9 protein comprises at least one preferably two, three, or four transmembrane domains, and/or at least one, preferably two pore loops (P-loops). Moreover, other biologically active portions, in which other regions of the protein are deleted, can be prepared by recombinant techniques and evaluated for one or more of the functional activities of a native TWIK-9 protein.

Another aspect of the invention features fragments of the protein having the amino acid sequence of SEQ ID NO:41, for example, for use as immunogens. In one embodiment, a fragment comprises at least 5 amino acids (e.g., contiguous or consecutive amino acids) of the amino acid sequence of SEQ ID NO:41, or an amino acid sequence encoded by the DNA insert of the plasmid deposited with the ATCC as Accession Number ______. In another embodiment, a fragment comprises at least 10, 15, 20, 25, 30, 35, 40, 45, 50 or more amino acids (e.g, contiguous or consecutive amino acids) of the amino acid sequence of SEQ ID NO:41, or an amino acid sequence encoded by the DNA insert of the plasmid deposited with the ATCC as Accession Number ______.

In a preferred embodiment, a TWIK-9 protein has an amino acid sequence shown in SEQ ID NO:41. In other embodiments, the TWIK-9 protein is substantially identical to SEQ ID NO:41, and retains the functional activity of the protein of SEQ ID NO:41, yet differs in amino acid sequence due to natural allelic variation or mutagenesis, as described in detail in subsection I above. In another embodiment, the TWIK-9 protein is a protein which comprises an amino acid sequence at least about 50%, 55%, 60%, 65%, 70%, 75%, 80%, 85%, 90%, 91%, 92%, 93%, 94%, 95%, 96%, 97%, 98%, 99% or more identical to SEQ ID NO:41.

In another embodiment, the invention features a TWIK-9 protein which is encoded by a nucleic acid molecule consisting of a nucleotide sequence at least about 50%, 55%, 60%, 65%, 70%, 75%, 80%, 85%, 90%, 91%, 92%, 93%, 94%, 95%, 96%, 97%, 98%, 99% or more identical to a nucleotide sequence of SEQ ID NO:40 or 42, or a complement thereof. This invention further features a TWIK-9 protein which is encoded by a nucleic acid molecule consisting of a nucleotide sequence which hybridizes under stringent hybridization conditions to a complement of a nucleic acid molecule comprising the nucleotide sequence of SEQ ID NO:40 or 42, or a complement thereof.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the TWIK-9 amino acid sequence of SEQ ID NO:41 having 374 amino acid residues, at least 112, preferably at least 150, more preferably at least 187, even more preferably at least 224, and even more preferably at least 262, 299 or 337 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of Meyers and Miller (Comput. Appl. Biosci., 4:11-17 (1988)) which has been incorporated into the ALIGN program (version 2.0 or version 2.0U), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to TWIK-9 nucleic acid molecules of the invention. BLAST protein searches can be performed with the XBLAST program, score=50, wordlength=3 to obtain amino acid sequences homologous to TWIK-9 protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides TWIK-9 chimeric or fusion proteins. As used herein, a TWIK-9 “chimeric protein” or “fusion protein” comprises a TWIK-9 polypeptide operatively linked to a non-TWIK-9 polypeptide. A “TWIK-9 polypeptide” refers to a polypeptide having an amino acid sequence corresponding to TWIK-9, whereas a “non-TWIK-9 polypeptide” refers to a polypeptide having an amino acid sequence corresponding to a protein which is not substantially homologous to the TWIK-9 protein, e.g., a protein which is different from the TWIK-9 protein and which is derived from the same or a different organism. Within a TWIK-9 fusion protein the TWIK-9 polypeptide can correspond to all or a portion of a TWIK-9 protein. In a preferred embodiment, a TWIK-9 fusion protein comprises at least one biologically active portion of a TWIK-9 protein. In another preferred embodiment, a TWIK-9 fusion protein comprises at least two biologically active portions of a TWIK-9 protein. Within the fusion protein, the term “operatively linked” is intended to indicate that the TWIK-9 polypeptide and the non-TWIK-9 polypeptide are fused in-frame to each other. The non-TWIK-9 polypeptide can be fused to the N-terminus or C-terminus of the TWIK-9 polypeptide.

For example, in one embodiment, the fusion protein is a GST-TWIK-9 fusion protein in which the TWIK-9 sequences are fused to the C-terminus of the GST sequences. Such fusion proteins can facilitate the purification of recombinant TWIK-9. In another embodiment, the fusion protein is a TWIK-9 protein containing a heterologous signal sequence at its N-terminus. In certain host cells (e.g., mammalian host cells), expression and/or secretion of TWIK-9 can be increased through use of a heterologous signal sequence.

The TWIK-9 fusion proteins of the invention can be incorporated into pharmaceutical compositions and administered to a subject in vivo. The TWIK-9 fusion proteins can be used to affect the bioavailability of a TWIK-9 ligand and/or substrate. Use of TWIK-9 fusion proteins may be useful therapeutically for the treatment of disorders caused by, for example, (i) aberrant modification or mutation of a gene encoding a TWIK-9 protein; (ii) mis-regulation of the TWIK-9 gene; and (iii) aberrant post-translational modification of a TWIK-9 protein.

Moreover, the TWIK-9-fusion proteins of the invention can be used as immunogens to produce anti-TWIK-9 antibodies in a subject, to purify TWIK-9 substrates, and in screening assays to identify molecules which inhibit or enhance the interaction of TWIK-9 with a TWIK-9 ligand and/or substrate.

Preferably, a TWIK-9 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons:1992). Moreover, many expression vectors are commercially available that already encode a fusion moiety (e.g., a GST polypeptide). A TWIK-9-encoding nucleic acid can be cloned into such an expression vector such that the fusion moiety is linked in-frame to the TWIK-9 protein.

The present invention also pertains to variants of the TWIK-9 proteins which function as either TWIK-9 agonists (mimetics) or as TWIK-9 antagonists. Variants of the TWIK-9 proteins can be generated by mutagenesis, e.g., discrete point mutation or truncation of a TWIK-9 protein. An agonist of the TWIK-9 proteins can retain substantially the same, or a subset, of the biological activities of the naturally occurring form of a TWIK-9 protein. An antagonist of a TWIK-9 protein can inhibit one or more of the activities of the naturally occurring form of the TWIK-9 protein by, for example, competitively modulating a TWIK-9-mediated activity of a TWIK-9 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the TWIK-9 protein.

In one embodiment, variants of a TWIK-9 protein which function as either TWIK-9 agonists (mimetics) or as TWIK-9 antagonists can be identified by screening combinatorial libraries of mutants, e.g., truncation mutants, of a TWIK-9 protein for TWIK-9 protein agonist or antagonist activity. In one embodiment, a variegated library of TWIK-9 variants is generated by combinatorial mutagenesis at the nucleic acid level and is encoded by a variegated gene library. A variegated library of TWIK-9 variants can be produced by, for example, enzymatically ligating a mixture of synthetic oligonucleotides into gene sequences such that a degenerate set of potential TWIK-9 sequences is expressible as individual polypeptides, or alternatively, as a set of larger fusion proteins (e.g., for phage display) containing the set of TWIK-9 sequences therein. There are a variety of methods which can be used to produce libraries of potential TWIK-9 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TWIK-9 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a TWIK-9 protein coding sequence can be used to generate a variegated population of TWIK-9 fragments for screening and subsequent selection of variants of a TWIK-9 protein. In one embodiment, a library of coding sequence fragments can be generated by treating a double stranded PCR fragment of a TWIK-9 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the TWIK-9 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TWIK-9 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify TWIK-9 variants (Arkin and Youvan (1992) Proc. Natl. Acad. Sci. USA 89:7811-7815; Delagrave et al. (1993) Protein Eng. 6(3):327-331).

In one embodiment, cell based assays can be exploited to analyze a variegated TWIK-9 library. For example, a library of expression vectors can be transfected into a cell line, e.g, a neuronal cell line, which ordinarily responds to TWIK-9 in a particular TWIK-9 ligand-dependent manner. The transfected cells are then contacted with TWIK-9 and the effect of the expression of the mutant on, e.g., membrane excitability of TWIK-9 can be detected. Plasmid DNA can then be recovered from the cells which score for inhibition, or alternatively, potentiation of signaling by the TWIK-9 ligand, and the individual clones further characterized.

An isolated TWIK-9 protein, or a portion or fragment thereof, can be used as an immunogen to generate antibodies that bind TWIK-9 using standard techniques for polyclonal and monoclonal antibody preparation. A full-length TWIK-9 protein can be used or, alternatively, the invention provides antigenic peptide fragments of TWIK-9 for use as immunogens. The antigenic peptide of TWIK-9 comprises at least 8 amino acid residues of the amino acid sequence shown in SEQ ID NO:41 and encompasses an epitope of TWIK-9 such that an antibody raised against the peptide forms a specific immune complex with TWIK-9. Preferably, the antigenic peptide comprises at least 10 amino acid residues, more preferably at least 15 amino acid residues, even more preferably at least 20 amino acid residues, and most preferably at least 30 amino acid residues.

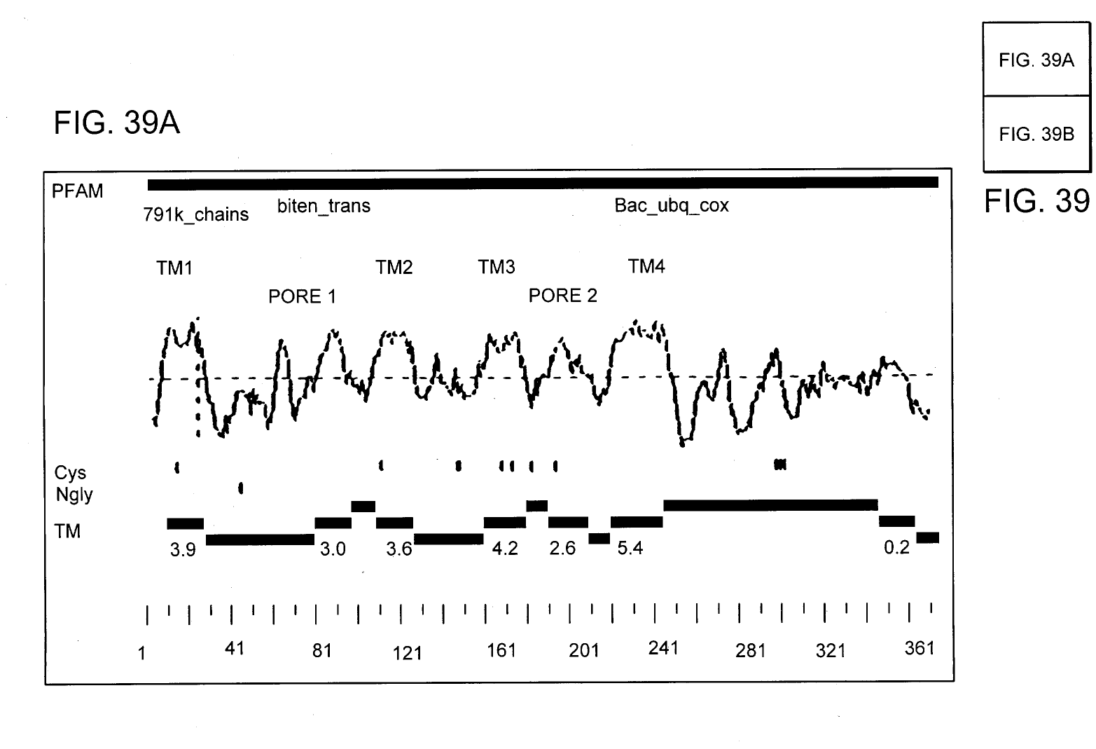

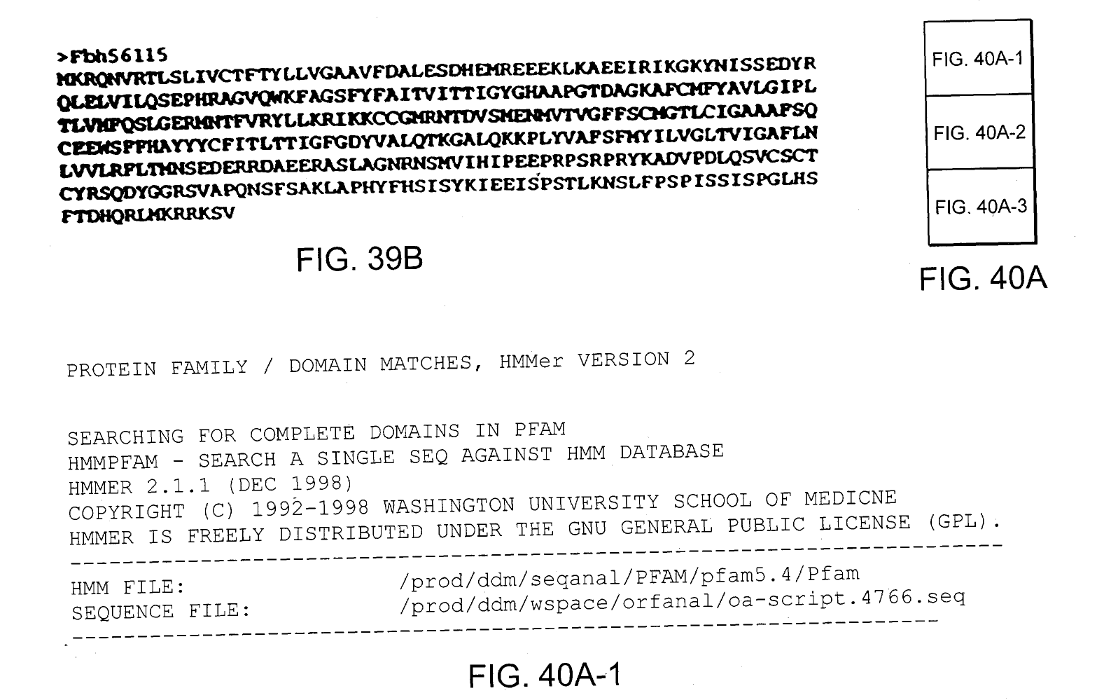

Preferred epitopes encompassed by the antigenic peptide are regions of TWIK-9 that are located on the surface of the protein, e.g., hydrophilic regions, as well as regions with high antigenicity (see, for example, FIG. 39).

A TWIK-9 immunogen typically is used to prepare antibodies by immunizing a suitable subject (e.g., rabbit, goat, mouse, or other mammal) with the immunogen. An appropriate immunogenic preparation can contain, for example, recombinantly expressed TWIK-9 protein or a chemically-synthesized TWIK-9 polypeptide. The preparation can further include an adjuvant, such as Freund's complete or incomplete adjuvant, or similar immunostimulatory agent. Immunization of a suitable subject with an immunogenic TWIK-9 preparation induces a polyclonal anti-TWIK-9 antibody response.

Accordingly, another aspect of the invention pertains to anti-TWIK-9 antibodies. The term “antibody” as used herein refers to immunoglobulin molecules and immunologically active portions of immunoglobulin molecules, i.e., molecules that contain an antigen binding site which specifically binds (immunoreacts with) an antigen, such as TWIK-9. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind TWIK-9. The term “monoclonal antibody” or “monoclonal antibody composition”, as used herein, refers to a population of antibody molecules that contain only one species of an antigen binding site capable of immunoreacting with a particular epitope of TWIK-9. A monoclonal antibody composition thus typically displays a single binding affinity for a particular TWIK-9 protein with which it immunoreacts.

Polyclonal anti-TWIK-9 antibodies can be prepared as described above by immunizing a suitable subject with a TWIK-9 immunogen. The anti-TWIK-9 antibody titer in the immunized subject can be monitored over time by standard techniques, such as with an enzyme linked immunosorbent assay (ELISA) using immobilized TWIK-9. If desired, the antibody molecules directed against TWIK-9 can be isolated from the mammal (e.g., from the blood) and further purified by well known techniques, such as protein A chromatography to obtain the IgG fraction. At an appropriate time after immunization, e.g., when the anti-TWIK-9 antibody titers are highest, antibody-producing cells can be obtained from the subject and used to prepare monoclonal antibodies by standard techniques, such as the hybridoma technique originally described by Kohler and Milstein (1975) Nature 256:495-497 (see also Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med., 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a TWIK-9 immunogen as described above, and the culture supernatants of the resulting hybridoma cells are screened to identify a hybridoma producing a monoclonal antibody that binds TWIK-9.

Any of the many well known protocols used for fusing lymphocytes and immortalized cell lines can be applied for the purpose of generating an anti-TWIK-9 monoclonal antibody (see, e.g., Galfre, G. et al. (1977) Nature 266:55052; Gefter et al. Somatic Cell Genet., supra; Lerner (1981) supra; Kenneth, Monoclonal Antibodies, supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma line These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind TWIK-9, e.g, using a standard ELISA assay.

Alternative to preparing monoclonal antibody-secreting hybridomas, a monoclonal anti-TWIK-9 antibody can be identified and isolated by screening a recombinant combinatorial immunoglobulin library (e.g., an antibody phage display library) with TWIK-9 to thereby isolate immunoglobulin library members that bind TWIK-9. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-TWIK-9 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-TWIK-9 antibody (e.g., monoclonal antibody) can be used to isolate TWIK-9 by standard techniques, such as affinity chromatography or immunoprecipitation. An anti-TWIK-9 antibody can facilitate the purification of natural TWIK-9 from cells and of recombinantly produced TWIK-9 expressed in host cells. Moreover, an anti-TWIK-9 antibody can be used to detect TWIK-9 protein (e.g., in a cellular lysate or cell supernatant) in order to evaluate the abundance and pattern of expression of the TWIK-9 protein. Anti-TWIK-9 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, for example recombinant expression vectors, containing a TWIK-9 nucleic acid molecule or vectors containing a nucleic acid molecule which encodes a TWIK-9 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g, in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., TWIK-9 proteins, mutant forms of TWIK-9 proteins, fusion proteins, and the like).

Accordingly, an exemplary embodiment provides a method for producing a protein, preferably a TWIK-9 protein, by culturing in a suitable medium a host cell of the invention (e.g., a mammalian host cell such as a non-human mammalian cell) containing a recombinant expression vector, such that the protein is produced.

The recombinant expression vectors of the invention can be designed for expression of TWIK-9 proteins in prokaryotic or eukaryotic cells. For example, TWIK-9 proteins can be expressed in bacterial cells such as E. coli, insect cells (using baculovirus expression vectors) yeast cells or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in TWIK-9 activity assays, (e.g., direct assays or competitive assays described in detail below), or to generate antibodies specific for TWIK-9 proteins, for example. In a preferred embodiment, a TWIK-9 fusion protein expressed in a retroviral expression vector of the present invention can be utilized to infect bone marrow cells, which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al. (1990) Methods Enzymol. 185:60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S. (1990) Methods Enzymol. 185:119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the TWIK-9 expression vector is a yeast expression vector. Examples of vectors for expression in yeast S. cerivisae include pYepSec1 (Baldari, et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and picZ (Invitrogen Corporation, San Diego, Calif.).

Alternatively, TWIK-9 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al. Molecular Cloning: A Laboratory Manual. 2^{nd}, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the alpha-myosin heavy chain promoter (cardiac specific), albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), adipose-specific promoters (U.S. Pat. No. 5,476,926; WO 92/06104), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the □-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to TWIK-9 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., “Antisense RNA as a molecular tool for genetic analysis”, Reviews—Trends in Genetics, Vol. 1(1) 1986.

Another aspect of the invention pertains to host cells into which a TWIK-9 nucleic acid molecule of the invention is introduced, e.g., a TWIK-9 nucleic acid molecule within a vector (e.g, a recombinant expression vector) or a TWIK-9 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a TWIK-9 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2^{nd}, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a TWIK-9 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a TWIK-9 protein. Accordingly, the invention further provides methods for producing a TWIK-9 protein using the host cells of the invention. In one embodiment, the method comprises culturing the host cell of the invention (into which a recombinant expression vector encoding a TWIK-9 protein has been introduced) in a suitable medium such that a TWIK-9 protein is produced. In another embodiment, the method further comprises isolating a TWIK-9 protein from the medium or the host cell.

The host cells of the invention can also be used to produce non-human transgenic animals. For example, in one embodiment, a host cell of the invention is a fertilized oocyte or an embryonic stem cell into which TWIK-9-coding sequences have been introduced. Such host cells can then be used to create non-human transgenic animals in which exogenous TWIK-9 sequences have been introduced into their genome or homologous recombinant animals in which endogenous TWIK-9 sequences have been altered. Such animals are useful for studying the function and/or activity of a TWIK-9 protein and for identifying and/or evaluating modulators of TWIK-9 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous TWIK-9 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a TWIK-9-encoding nucleic acid into the male pronuclei of a fertilized oocyte, e.g., by microinjection or retroviral infection, and allowing the oocyte to develop in a pseudopregnant female foster animal. The TWIK-9 cDNA sequence of SEQ ID NO:40 can be introduced as a transgene into the genome of a non-human animal. Alternatively, a non-human homologue of a human TWIK-9 gene, such as a rat or mouse TWIK-9 gene, can be used as a transgene. Alternatively, a TWIK-9 gene homologue, such as another TWIK-9 family member, can be isolated based on hybridization to the TWIK-9 cDNA sequences of SEQ ID NO:40 or 42, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to a TWIK-9 transgene to direct expression of a TWIK-9 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a TWIK-9 transgene in its genome and/or expression of TWIK-9 mRNA in tissues or cells of the animals. A transgenic founder animal can then be used to breed additional animals carrying the transgene. Moreover, transgenic animals carrying a transgene encoding a TWIK-9 protein can further be bred to other transgenic animals carrying other transgenes.

To create a homologous recombinant animal, a vector is prepared which contains at least a portion of a TWIK-9 gene into which a deletion, addition or substitution has been introduced to thereby alter, e.g., functionally disrupt, the TWIK-9 gene. The TWIK-9 gene can be a human gene (e.g., the cDNA of SEQ ID NO:42), but more preferably, is a non-human homologue of a human TWIK-9 gene (e.g., a cDNA isolated by stringent hybridization with the nucleotide sequence of SEQ ID NO:40), For example, a mouse TWIK-9 gene can be used to construct a homologous recombination nucleic acid molecule, e.g., a vector, suitable for altering an endogenous TWIK-9 gene in the mouse genome. In a preferred embodiment, the homologous recombination nucleic acid molecule is designed such that, upon homologous recombination, the endogenous TWIK-9 gene is functionally disrupted (i.e., no longer encodes a functional protein; also referred to as a “knock out” vector). Alternatively, the homologous recombination nucleic acid molecule can be designed such that, upon homologous recombination, the endogenous TWIK-9 gene is mutated or otherwise altered but still encodes functional protein (e.g., the upstream regulatory region can be altered to thereby alter the expression of the endogenous TWIK-9 protein). In the homologous recombination nucleic acid molecule, the altered portion of the TWIK-9 gene is flanked at its 5′ and 3′ ends by additional nucleic acid sequence of the TWIK-9 gene to allow for homologous recombination to occur between the exogenous TWIK-9 gene carried by the homologous recombination nucleic acid molecule and an endogenous TWIK-9 gene in a cell, e.g., an embryonic stem cell. The additional flanking TWIK-9 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced TWIK-9 gene has homologously recombined with the endogenous TWIK-9 gene are selected (see e.g., Li, E. et al. (1992) Cell 69:915). The selected cells can then be injected into a blastocyst of an animal (e.g., a mouse) to form aggregation chimeras (see e.g., Bradley, A. in Teratocarcinomas and Embryonic Stem Cells: A Practical Approach, Robertson, E. J. ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The TWIK-9 nucleic acid molecules, TWIK-9 proteins, fragments thereof, anti-TWIK-9 antibodies, and TWIK-9 modulators (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a TWIK-9 protein or an anti-TWIK-9 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

In certain embodiments of the invention, a modulator of TWIK-9 activity is administered in combination with other agents (e.g., a small molecule), or in conjunction with another, complementary treatment regime. For example, in one embodiment, a modulator of TWIK-9 activity is used to treat a central nervous system disorder. Accordingly, modulation of TWIK-9 activity may be used in conjunction with, for example, neuroactive and/or neurotrophic agents.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, protein fragments, antibodies, peptides, peptidomimetics, and small molecules described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a TWIK-9 protein of the invention has one or more of the following activities: (1) interacting with a non-TWIK protein molecule; (2) activating a TWIK-dependent signal transduction pathway; (3) modulating the release of neurotransmitters; (4) modulating membrane excitability; (5) influencing the resting potential of membranes, wave forms and frequencies of action potentials, and thresholds of excitation, (6) modulating processes which underlie learning and memory, such as integration of sub-threshold synaptic responses and the conductance of back-propagating action potentials, and (7) mediating nociception.

The isolated nucleic acid molecules of the invention can be used, for example, to express TWIK-9 protein (e.g., via a recombinant expression vector in a host cell in gene therapy applications), to detect TWIK-9 mRNA (e.g., in a biological sample) or a genetic alteration in a TWIK-9 gene, and to modulate TWIK-9 activity, as described further below. The TWIK-9 proteins can be used to treat disorders characterized by insufficient or excessive production of a TWIK-9 ligand or substrate, or production of TWIK-9 inhibitors, for example, potassium channel associated disorders.

As used interchangeably herein, an “potassium channel-associated disorder” or a “TWIK-9-associated disorder” includes a disorder, disease or condition which is caused or characterized by a misregulation (e.g., downregulation or upregulation) of a potassium channel activity or a TWIK-9-mediated activity. Potassium channel-associated disorders can detrimentally affect cellular functions such as cellular proliferation, growth, differentiation, and/or migration; inter- or intra-cellular communication; tissue function, such as muscular function or cardiac function; systemic responses in an organism, such as nervous system responses.

Examples of potassium channel associated disorders include central nervous system (CNS) disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, movement disorders, progressive supranuclear palsy, epilepsy, AIDS related dementia, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further examples of potassium channel associated disorders include cardiac-related disorders. Cardiovascular system disorders in which the TWIK-9 molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, cerebral ischemia, stroke, coronary artery spasm, and arrhythmia. TWIK-9-mediated or related disorders also include disorders of the musculoskeletal system such as paralysis and muscle weakness, e.g., ataxia, myotonia, and myokymia.

Further disorders in which the TWIK-9 molecules of the invention may be involved are pain disorders. Pain disorders include those that affect pain signaling mechanisms. As used herein, the term “pain signaling mechanisms” includes the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter sensory neurons. These sensory neurons transmit the information to the central nervous system, evoking a perception of pain or discomfort and initiating appropriate protective reflexes. The TWIK-9 molecules of the present invention may be present on these sensory neurons and, thus, may be involved in detecting these noxious chemical, mechanical, or thermal stimuli and transducing this information into membrane depolarization events. Thus, the TWIK-9 molecules by participating in pain signaling mechanisms, may modulate pain elicitation and act as targets for developing novel diagnostic targets and therapeutic agents to control pain. Examples of pain disorders include headache (e.g., tension headache or migraine), back pain, cancer pain, arthritis pain, or neurogenic pain.

Potassium channel disorders also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The TWIK-9 molecules of the present invention are involved in signal transduction mechanisms, which are known to be involved in cellular growth, differentiation, and migration processes. Thus, the TWIK-9 molecules may modulate cellular growth, differentiation, or migration, and may play a role in disorders characterized by aberrantly regulated growth, differentiation, or migration. Such disorders include cancer, e.g., carcinoma, sarcoma, lymphoma or leukemia, examples of which include, but are not limited to, breast, endometrial, ovarian, uterine, hepatic, gastrointestinal, prostate, colorectal, and lung cancer, melanoma, neurofibromatosis, adenomatous polyposis of the colon, Wilms' tumor, nephroblastoma, teratoma, rhabdomyosarcoma; tumor invasion, angiogenesis and metastasis; skeletal dysplasia; neuronal deficiencies resulting from impaired neural induction and patterning; and hematopoietic and/or myeloproliferative disorders.

TWIK-9-associated or related disorders also include disorders affecting tissues in which TWIK-9 protein is expressed.

In addition, the TWIK-9 proteins can be used to screen for naturally occurring TWIK-9 ligands or substrates, to screen for drugs or compounds which modulate TWIK-9 activity, as well as to treat disorders characterized by insufficient or excessive production of TWIK-9 protein or production of TWIK-9 protein forms which have decreased, aberrant or unwanted activity compared to TWIK-9 wild type protein (e.g., potassium channel associated disorders).

Moreover, the anti-TWIK-9 antibodies of the invention can be used to detect and isolate TWIK-9 proteins, regulate the bioavailability of TWIK-9 proteins, and modulate TWIK-9 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to TWIK-9 proteins, have a stimulatory or inhibitory effect on, for example, TWIK-9 expression or TWIK-9 activity, or have a stimulatory or inhibitory effect on, for example, the expression or activity of a TWIK-9 ligand or substrate.

In one embodiment, the invention provides assays for screening candidate or test compounds which are ligands or substrates of a TWIK-9 protein or polypeptide or biologically active portion thereof. In another embodiment, the invention provides assays for screening candidate or test compounds which bind to or modulate the activity of a TWIK-9 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:45).

Examples of methods for the synthesis of molecular libraries can be found in the art, for example, in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, an assay is a cell-based assay in which a cell which expresses a TWIK-9 protein or biologically active portion thereof is contacted with a test compound and the ability of the test compound to modulate TWIK-9 activity is determined. Determining the ability of the test compound to modulate TWIK-9 activity can be accomplished by monitoring, for example, potassium current, neurotransmitter release and/or membrane excitability in a cell which expresses TWIK-9. The cell, for example, can be of mammalian origin, e.g., a neuronal cell.

The ability of the test compound to modulate TWIK-9 binding to a ligand or substrate, or to bind to TWIK-9 can also be determined. Determining the ability of the test compound to modulate TWIK-9 binding to a ligand or substrate can be accomplished, for example, by coupling the TWIK-9 ligand or substrate with a radioisotope or enzymatic label such that binding of the TWIK-9 ligand or substrate to TWIK-9 can be determined by detecting the labeled TWIK-9 ligand or substrate in a complex. Alternatively, TWIK-9 could be coupled with a radioisotope or enzymatic label to monitor the ability of a test compound to modulate TWIK-9 binding to a TWIK-9 ligand or substrate in a complex. Determining the ability of the test compound to bind TWIK-9 can be accomplished, for example, by coupling the compound with a radioisotope or enzymatic label such that binding of the compound to TWIK-9 can be determined by detecting the labeled compound in a complex. For example, compounds (e.g., TWIK-9 ligands or substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a TWIK-9 ligand or substrate) to interact with TWIK-9 without the labeling of any of the interactants. For example, a microphysiometer can be used to detect the interaction of a compound with TWIK-9 without the labeling of either the compound or the TWIK-9. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and TWIK-9.

In another embodiment, an assay is a cell-based assay comprising contacting a cell expressing a TWIK-9 target molecule (e.g., a TWIK-9 substrate) with a test compound and determining the ability of the test compound to modulate (e.g. stimulate or inhibit) the activity of the TWIK-9 target molecule. Determining the ability of the test compound to modulate the activity of a TWIK-9 target molecule can be accomplished, for example, by determining the ability of the TWIK-9 protein to bind to or interact with the TWIK-9 target molecule.

Determining the ability of the TWIK-9 protein or a biologically active fragment thereof, to bind to or interact with a TWIK-9 target molecule can be accomplished by one of the methods described above for determining direct binding. In a preferred embodiment, determining the ability of the TWIK-9 protein to bind to or interact with a TWIK-9 target molecule can be accomplished by determining the activity of the target molecule. For example, the activity of the target molecule can be determined by detecting induction of a cellular response (e.g., changes in intracellular K⁺ levels), detecting catalytic/enzymatic activity of the target molecule upon an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which a TWIK-9 protein or biologically active portion thereof is contacted with a test compound and the ability of the test compound to bind to the TWIK-9 protein or biologically active portion thereof is determined. Preferred biologically active portions of the TWIK-9 proteins to be used in assays of the present invention include fragments which participate in interactions with non-TWIK-9 molecules, e.g., fragments with high surface probability scores (see, for example, FIG. 39). Binding of the test compound to the TWIK-9 protein can be determined either directly or indirectly as described above. In a preferred embodiment, the assay includes contacting the TWIK-9 protein or biologically active portion thereof with a known compound which binds TWIK-9 to form an assay mixture, contacting the assay mixture with a test compound, and determining the ability of the test compound to interact with a TWIK-9 protein, wherein determining the ability of the test compound to interact with a TWIK-9 protein comprises determining the ability of the test compound to preferentially bind to TWIK-9 or biologically active portion thereof as compared to the known compound.

In another embodiment, the assay is a cell-free assay in which a TWIK-9 protein or biologically active portion thereof is contacted with a test compound and the ability of the test compound to modulate (e.g., stimulate or inhibit) the activity of the TWIK-9 protein or biologically active portion thereof is determined. Determining the ability of the test compound to modulate the activity of a TWIK-9 protein can be accomplished, for example, by determining the ability of the TWIK-9 protein to bind to a TWIK-9 target molecule by one of the methods described above for determining direct binding. Determining the ability of the TWIK-9 protein to bind to a TWIK-9 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a TWIK-9 protein can be accomplished by determining the ability of the TWIK-9 protein to further modulate the activity of a downstream effector of a TWIK-9 target molecule. For example, the activity of the effector molecule on an appropriate target can be determined or the binding of the effector to an appropriate target can be determined as previously described.

In yet another embodiment, the cell-free assay involves contacting a TWIK-9 protein or biologically active portion thereof with a known compound which binds the TWIK-9 protein to form an assay mixture, contacting the assay mixture with a test compound, and determining the ability of the test compound to interact with the TWIK-9 protein, wherein determining the ability of the test compound to interact with the TWIK-9 protein comprises determining the ability of the TWIK-9 protein to preferentially bind to or modulate the activity of a TWIK-9 target molecule.

The cell-free assays of the present invention are amenable to use of both soluble and/or membrane-bound forms of isolated proteins (e.g., TWIK-9 proteins or biologically active portions thereof). In the case of cell-free assays in which a membrane-bound form of an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either TWIK-9 or its target molecule to facilitate separation of complexed from uncomplexed forms of one or both of the proteins, as well as to accommodate automation of the assay. Binding of a test compound to a TWIK-9 protein, or interaction of a TWIK-9 protein with a substrate or target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/TWIK-9 fusion proteins or glutathione-S-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or TWIK-9 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of TWIK-9 binding or activity determined using standard techniques.

Other techniques for immobilizing proteins on matrices can also be used in the screening assays of the invention. For example, either a TWIK-9 protein or a TWIK-9 substrate or target molecule can be immobilized utilizing conjugation of biotin and streptavidin. Biotinylated TWIK-9 protein, substrates, or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with TWIK-9 protein or target molecules but which do not interfere with binding of the TWIK-9 protein to its target molecule can be derivatized to the wells of the plate, and unbound target or TWIK-9 protein trapped in the wells by antibody conjugation. Methods for detecting such complexes, in addition to those described above for the GST-immobilized complexes, include immunodetection of complexes using antibodies reactive with the TWIK-9 protein or target molecule, as well as enzyme-linked assays which rely on detecting an enzymatic activity associated with the TWIK-9 protein or target molecule.

In another embodiment, modulators of TWIK-9 expression are identified in a method wherein a cell is contacted with a candidate compound and the expression of TWIK-9 mRNA or protein in the cell is determined. The level of expression of TWIK-9 mRNA or protein in the presence of the candidate compound is compared to the level of expression of TWIK-9 mRNA or protein in the absence of the candidate compound. The candidate compound can then be identified as a modulator of TWIK-9 expression based on this comparison. For example, when expression of TWIK-9 mRNA or protein is greater (statistically significantly greater) in the presence of the candidate compound than in its absence, the candidate compound is identified as a stimulator of TWIK-9 mRNA or protein expression. Alternatively, when expression of TWIK-9 mRNA or protein is less (statistically significantly less) in the presence of the candidate compound than in its absence, the candidate compound is identified as an inhibitor of TWIK-9 mRNA or protein expression. The level of TWIK-9 mRNA or protein expression in the cells can be determined by methods described herein for detecting TWIK-9 mRNA or protein.

In yet another aspect of the invention, the TWIK-9 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300) to identify other proteins which bind to or interact with TWIK-9 (“TWIK-9-binding proteins” or “TWIK-9-bp”) and are involved in TWIK-9 activity. Such TWIK-9-binding proteins are also likely to be involved in the propagation of signals by the TWIK-9 proteins or TWIK-9 targets as, for example, downstream elements of a TWIK-9-mediated signaling pathway. Alternatively, such TWIK-9-binding proteins may be TWIK-9 inhibitors.

The two-hybrid system is based on the modular nature of most transcription factors, which consist of separable DNA-binding and activation domains. Briefly, the assay utilizes two different DNA constructs. In one construct, the gene that codes for a TWIK-9 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TWIK-9-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the TWIK-9 protein.

In another aspect, the invention pertains to a combination of two or more of the assays described herein. For example, a modulating agent can be identified using a cell-based or a cell-free assay, and the ability of the agent to modulate the activity of a TWIK-9 protein can be confirmed in vivo, e.g., in an animal such as an animal model for a neurodegenerative disorder or for pain.

This invention further pertains to novel agents identified by the above-described screening assays. Accordingly, it is within the scope of this invention to further use an agent identified as described herein in an appropriate animal model. For example, an agent identified as described herein (e.g., a TWIK-9 modulating agent, an antisense TWIK-9 nucleic acid molecule, a TWIK-9-specific antibody, or a TWIK-9 binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the TWIK-9 nucleotide sequences, described herein, can be used to map the location of the TWIK-9 genes on a chromosome. The mapping of the TWIK-9 sequences to chromosomes is an important first step in correlating these sequences with genes associated with disease.

Briefly, TWIK-9 genes can be mapped to chromosomes by preparing PCR primers (preferably 15-25 bp in length) from the TWIK-9 nucleotide sequences. Computer analysis of the TWIK-9 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the TWIK-9 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the TWIK-9 nucleotide sequences to design oligonucleotide primers, sublocalization can be achieved with panels of fragments from specific chromosomes. Other mapping strategies which can similarly be used to map a TWIK-9 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome-specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in McKusick, V., Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the TWIK-9 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The TWIK-9 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the TWIK-9 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The TWIK-9 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:40 can comfortably provide positive individual identification with a panel of perhaps 10 to 1,000 primers which each yield a noncoding amplified sequence of 100 bases. If predicted coding sequences, such as those in SEQ ID NO:42 are used, a more appropriate number of primers for positive individual identification would be 500-2,000.

If a panel of reagents from TWIK-9 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial TWIK-9 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:40 are particularly appropriate for this use as greater numbers of polymorphisms occur in the noncoding regions, making it easier to differentiate individuals using this technique. Examples of polynucleotide reagents include the TWIK-9 nucleotide sequences or portions thereof, e.g., fragments derived from the noncoding regions of SEQ ID NO:40 having a length of at least 20 bases, preferably at least 30 bases.

The TWIK-9 nucleotide sequences described herein can further be used to provide polynucleotide reagents, e.g., labeled or labelable probes which can be used in, for example, an in situ hybridization technique, to identify a specific tissue, e.g., a tissue which expresses TWIK-9, e.g., brain tissue. This can be very useful in cases where a forensic pathologist is presented with a tissue of unknown origin. Panels of such TWIK-9 probes can be used to identify tissue by species and/or by organ type.

In a similar fashion, these reagents, e.g., TWIK-9 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining TWIK-9 protein and/or nucleic acid expression as well as TWIK-9 activity, in the context of a biological sample (e.g., blood, serum, cells, or tissue) to thereby determine whether an individual is afflicted with a disease or disorder, or is at risk of developing a disorder, associated with aberrant or unwanted TWIK-9 expression or activity. The invention also provides for prognostic (or predictive) assays for determining whether an individual is at risk of developing a disorder associated with TWIK-9 protein, nucleic acid expression, or activity. For example, mutations in a TWIK-9 gene can be assayed in a biological sample. Such assays can be used for prognostic or predictive purpose to thereby prophylactically treat an individual prior to the onset of a disorder characterized by or associated with TWIK-9 protein, nucleic acid expression or activity.

Another aspect of the invention pertains to monitoring the influence of agents (e.g., drugs, compounds) on the expression or activity of TWIK-9 in clinical trials.

These and other agents are described in further detail in the following sections.

1. Diagnostic Assays

An exemplary method for detecting the presence or absence of TWIK-9 protein, polypeptide or nucleic acid in a biological sample involves obtaining a biological sample from a test subject and contacting the biological sample with a compound or an agent capable of detecting TWIK-9 protein, polypeptide or nucleic acid (e.g., mRNA, genomic DNA) that encodes TWIK-9 protein such that the presence of TWIK-9 protein or nucleic acid is detected in the biological sample. In another aspect, the present invention provides a method for detecting the presence of TWIK-9 activity in a biological sample by contacting the biological sample with an agent capable of detecting an indicator of TWIK-9 activity such that the presence of TWIK-9 activity is detected in the biological sample. A preferred agent for detecting TWIK-9 mRNA or genomic DNA is a labeled nucleic acid probe capable of hybridizing to TWIK-9 mRNA or genomic DNA. The nucleic acid probe can be, for example, a full-length TWIK-9 nucleic acid, such as the nucleic acid of SEQ ID NO:40 or 42, or the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to TWIK-9 mRNA or genomic DNA. Other suitable probes for use in the diagnostic assays of the invention are described herein.

A preferred agent for detecting TWIK-9 protein is an antibody capable of binding to TWIK-9 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect TWIK-9 mRNA, protein, or genomic DNA in a biological sample in vitro as well as in vivo. For example, in vitro techniques for detection of TWIK-9 mRNA include Northern hybridizations and in situ hybridizations. In vitro techniques for detection of TWIK-9 protein include enzyme linked immunosorbent assays (ELISAs), Western blots, immunoprecipitations and immunofluorescence. In vitro techniques for detection of TWIK-9 genomic DNA include Southern hybridizations. Furthermore, in vivo techniques for detection of a TWIK-9 protein include introducing into a subject a labeled anti-TWIK-9 antibody. For example, the antibody can be labeled with a radioactive marker whose presence and location in a subject can be detected by standard imaging techniques.

The present invention also provides diagnostic assays for identifying the presence or absence of a genetic alteration characterized by at least one of (i) aberrant modification or mutation of a gene encoding a TWIK-9 protein; (ii) aberrant expression of a gene encoding a TWIK-9 protein; (iii) mis-regulation of the gene; and (iv) aberrant post-translational modification of a TWIK-9 protein, wherein a wild-type form of the gene encodes a protein with a TWIK-9 activity. “Misexpression or aberrant expression”, as used herein, refers to a non-wild type pattern of gene expression, at the RNA or protein level. It includes, but is not limited to, expression at non-wild type levels (e.g., over or under expression); a pattern of expression that differs from wild type in terms of the time or stage at which the gene is expressed (e.g., increased or decreased expression (as compared with wild type) at a predetermined developmental period or stage); a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene (e.g., a pattern of increased or decreased expression (as compared with wild type) in the presence of an increase or decrease in the strength of the stimulus).

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting TWIK-9 protein, mRNA, or genomic DNA, such that the presence of TWIK-9 protein, mRNA or genomic DNA is detected in the biological sample, and comparing the presence of TWIK-9 protein, mRNA or genomic DNA in the control sample with the presence of TWIK-9 protein, mRNA or genomic DNA in the test sample.

The invention also encompasses kits for detecting the presence of TWIK-9 in a biological sample. For example, the kit can comprise a labeled compound or agent capable of detecting TWIK-9 protein or mRNA in a biological sample; means for determining the amount of TWIK-9 in the sample; and means for comparing the amount of TWIK-9 in the sample with a standard. The compound or agent can be packaged in a suitable container. The kit can further comprise instructions for using the kit to detect TWIK-9 protein or nucleic acid.

2. Prognostic Assays

The diagnostic methods described herein can furthermore be utilized to identify subjects having or at risk of developing a disease or disorder associated with aberrant or unwanted TWIK-9 expression or activity. As used herein, the term “aberrant” includes a TWIK-9 expression or activity which deviates from the wild type TWIK-9 expression or activity. Aberrant expression or activity includes increased or decreased expression or activity, as well as expression or activity which does not follow the wild type developmental pattern of expression or the subcellular pattern of expression. For example, aberrant TWIK-9 expression or activity is intended to include the cases in which a mutation in the TWIK-9 gene causes the TWIK-9 gene to be under-expressed or over-expressed and situations in which such mutations result in a non-functional TWIK-9 protein or a protein which does not function in a wild-type fashion, e.g., a protein which does not interact with a TWIK-9 ligand or substrate, e.g., a non-potassium channel subunit, or one which interacts with a non-TWIK-9 ligand or substrate. As used herein, the term “unwanted” includes an unwanted phenomenon involved in a biological response such as deregulated cell proliferation or seizure susceptibility. For example, the term unwanted includes a TWIK-9 expression or activity which is undesirable in a subject.

The assays described herein, such as the preceding diagnostic assays or the following assays, can be utilized to identify a subject having or at risk of developing a disorder associated with a misregulation in TWIK-9 protein activity or nucleic acid expression, such as a CNS disorder (e.g., a cognitive or neurodegenerative disorder, or a pain disorder), a muscular disorder, a cardiovascular disorder, or a cellular proliferation, growth, differentiation or migration disorder. Alternatively, the prognostic assays can be utilized to identify a subject having or at risk for developing a disorder associated with a misregulation in TWIK-9 protein activity or nucleic acid expression, such as a CNS disorder (e.g., a cognitive or neurodegenerative disorder, or a pain disorder), a muscular disorder, a cardiovascular disorder, or a cellular proliferation, growth, differentiation or migration disorder. Thus, the present invention provides a method for identifying a disease or disorder associated with aberrant or unwanted TWIK-9 expression or activity in which a test sample is obtained from a subject and TWIK-9 protein or nucleic acid (e.g., mRNA or genomic DNA) is detected, wherein the presence of TWIK-9 protein or nucleic acid is diagnostic for a subject having or at risk of developing a disease or disorder associated with aberrant or unwanted TWIK-9 expression or activity. As used herein, a “test sample” refers to a biological sample obtained from a subject of interest. For example, a test sample can be a biological fluid (e.g., cerebrospinal fluid or serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted TWIK-9 expression or activity. For example, such methods can be used to determine whether a subject can be effectively treated with an agent for a CNS disorder (e.g., a cognitive or neurodegenerative disorder, or a pain disorder), a muscular disorder, a cardiovascular disorder, or a cellular proliferation, growth, differentiation or migration disorder. Thus, the present invention provides methods for determining whether a subject can be effectively treated with an agent for a disorder associated with aberrant or unwanted TWIK-9 expression or activity in which a test sample is obtained and TWIK-9 protein or nucleic acid expression or activity is detected (e.g., wherein the abundance of TWIK-9 protein or nucleic acid expression or activity is diagnostic for a subject that can be administered the agent to treat a disorder associated with aberrant or unwanted TWIK-9 expression or activity).

The methods of the invention can also be used to detect genetic alterations in a TWIK-9 gene, thereby determining if a subject with the altered gene is at risk for a disorder characterized by misregulation in TWIK-9 protein activity or nucleic acid expression, such as a CNS disorder (e.g., a cognitive or neurodegenerative disorder, or a pain disorder), a muscular disorder, a cardiovascular disorder, or a cellular proliferation, growth, differentiation or migration disorder. In preferred embodiments, the methods include detecting, in a sample of cells from the subject, the presence or absence of a genetic alteration characterized by at least one of an alteration affecting the integrity of a gene encoding a TWIK-9-protein, or the mis-expression of the TWIK-9 gene. For example, such genetic alterations can be detected by ascertaining the existence of at least one of 1) a deletion of one or more nucleotides from a TWIK-9 gene; 2) an addition of one or more nucleotides to a TWIK-9 gene; 3) a substitution of one or more nucleotides of a TWIK-9 gene, 4) a chromosomal rearrangement of a TWIK-9 gene; 5) an alteration in the level of a messenger RNA transcript of a TWIK-9 gene, 6) aberrant modification of a TWIK-9 gene, such as of the methylation pattern of the genomic DNA, 7) the presence of a non-wild type splicing pattern of a messenger RNA transcript of a TWIK-9 gene, 8) a non-wild type level of a TWIK-9-protein, 9) allelic loss of a TWIK-9 gene, and 10) inappropriate post-translational modification of a TWIK-9-protein. As described herein, there are a large number of assays known in the art which can be used for detecting alterations in a TWIK-9 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the TWIK-9-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a TWIK-9 gene under conditions such that hybridization and amplification of the TWIK-9-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a TWIK-9 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in TWIK-9 can be identified by hybridizing a sample and control nucleic acids, e.g., DNA or RNA, to high density arrays containing hundreds or thousands of oligonucleotides probes (Cronin, M. T. et al. (1996) Human Mutation 7:244-255; Kozal, M. J. et al. (1996) Nature Medicine 2:753-759). For example, genetic mutations in TWIK-9 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the TWIK-9 gene and detect mutations by comparing the sequence of the sample TWIK-9 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the TWIK-9 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type TWIK-9 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in TWIK-9 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a TWIK-9 sequence, e.g, a wild-type TWIK-9 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in TWIK-9 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control TWIK-9 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a TWIK-9 gene.

Furthermore, any cell type or tissue in which TWIK-9 is expressed may be utilized in the prognostic assays described herein.

3. Monitoring of Effects During Clinical Trials

Monitoring the influence of agents (e.g., drugs) on the expression or activity of a TWIK-9 protein (e.g., the modulation of pain signaling mechanisms, neurotransmitter release, membrane excitability and/or cell growth, proliferation, or differentiation mechanisms) can be applied not only in basic drug screening, but also in clinical trials. For example, the effectiveness of an agent determined by a screening assay as described herein to increase TWIK-9 gene expression, protein levels, or upregulate TWIK-9 activity, can be monitored in clinical trials of subjects exhibiting decreased TWIK-9 gene expression, protein levels, or downregulated TWIK-9 activity. Alternatively, the effectiveness of an agent determined by a screening assay to decrease TWIK-9 gene expression, protein levels, or downregulate TWIK-9 activity, can be monitored in clinical trials of subjects exhibiting increased TWIK-9 gene expression, protein levels, or upregulated TWIK-9 activity. In such clinical trials, the expression or activity of a TWIK-9 gene, and preferably, other genes that have been implicated in, for example, a TWIK-9-associated disorder can be used as a “read out” or markers of the phenotype of a particular cell.

For example, and not by way of limitation, genes, including TWIK-9, that are modulated in cells by treatment with an agent (e.g., compound, drug or small molecule) which modulates TWIK-9 activity (e.g., identified in a screening assay as described herein) can be identified. Thus, to study the effect of agents on TWIK-9-associated disorders (e.g., disorders characterized by deregulated pain signaling mechanisms, K⁺ currents, neurotransmitter release, membrane excitability, and/or cell growth, proliferation, or differentiation mechanisms), for example, in a clinical trial, cells can be isolated and RNA prepared and analyzed for the levels of expression of TWIK-9 and other genes implicated in the TWIK-9-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of TWIK-9 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a TWIK-9 protein, mRNA, or genomic DNA in the preadministration sample; (iii) obtaining one or more post-administration samples from the subject; (iv) detecting the level of expression or activity of the TWIK-9 protein, mRNA, or genomic DNA in the post-administration samples; (v) comparing the level of expression or activity of the TWIK-9 protein, mRNA, or genomic DNA in the pre-administration sample with the TWIK-9 protein, mRNA, or genomic DNA in the post administration sample or samples; and (vi) altering the administration of the agent to the subject accordingly. For example, increased administration of the agent may be desirable to increase the expression or activity of TWIK-9 to higher levels than detected, i.e., to increase the effectiveness of the agent. Alternatively, decreased administration of the agent may be desirable to decrease expression or activity of TWIK-9 to lower levels than detected, i.e. to decrease the effectiveness of the agent. According to such an embodiment, TWIK-9 expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a TWIK-9-associated disorder, e.g., a disorder associated with aberrant or unwanted TWIK-9 expression or activity, e.g., a potassium channel associated disorder such as a CNS disorder, a pain disorder, a cardiovascular disorder, or a cell proliferation, growth or differentiation disorder. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the TWIK-9 molecules of the present invention or TWIK-9 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted TWIK-9 expression or activity, by administering to the subject a TWIK-9 or an agent which modulates TWIK-9 expression or at least one TWIK-9 activity. Subjects at risk for a disease which is caused or contributed to by aberrant or unwanted TWIK-9 expression or activity can be identified by, for example, any or a combination of diagnostic or prognostic assays as described herein. Administration of a prophylactic agent can occur prior to the manifestation of symptoms characteristic of the TWIK-9 aberrancy, such that a disease or disorder is prevented or, alternatively, delayed in its progression. Depending on the type of TWIK-9 aberrancy, for example, a TWIK-9, TWIK-9 agonist or TWIK-9 antagonist agent can be used for treating the subject. The appropriate agent can be determined based on screening assays described herein.

2. Therapeutic Methods

Another aspect of the invention pertains to methods of modulating TWIK-9 expression or activity for therapeutic purposes. Accordingly, in an exemplary embodiment, the modulatory method of the invention involves contacting a cell capable of expressing TWIK-9 with an agent that modulates one or more of the activities of TWIK-9 protein activity associated with the cell, such that TWIK-9 activity in the cell is modulated. An agent that modulates TWIK-9 protein activity can be an agent as described herein, such as a nucleic acid or a protein, a naturally-occurring target molecule of a TWIK-9 protein (e.g., a TWIK-9 ligand or substrate), a TWIK-9 antibody, a TWIK-9 agonist or antagonist, a peptidomimetic of a TWIK-9 agonist or antagonist, or other small molecule. In one embodiment, the agent stimulates one or more TWIK-9 activities. Examples of such stimulatory agents include active TWIK-9 protein and a nucleic acid molecule encoding TWIK-9 that has been introduced into the cell. In another embodiment, the agent inhibits one or more TWIK-9 activities. Examples of such inhibitory agents include antisense TWIK-9 nucleic acid molecules, anti-TWIK-9 antibodies, and TWIK-9 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a TWIK-9 protein or nucleic acid molecule. In one embodiment, the method involves administering an agent (e.g., an agent identified by a screening assay described herein), or combination of agents that modulates (e.g., upregulates or downregulates) TWIK-9 expression or activity. In another embodiment, the method involves administering a TWIK-9 protein or nucleic acid molecule as therapy to compensate for reduced, aberrant, or unwanted TWIK-9 expression or activity.

The term “treatment” as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

Stimulation of TWIK-9 activity is desirable in situations in which TWIK-9 is abnormally downregulated and/or in which increased TWIK-9 activity is likely to have a beneficial effect. Likewise, inhibition of TWIK-9 activity is desirable in situations in which TWIK-9 is abnormally upregulated and/or in which decreased TWIK-9 activity is likely to have a beneficial effect.

3. Pharmacogenomics

The TWIK-9 molecules of the present invention, as well as agents, or modulators which have a stimulatory or inhibitory effect on TWIK-9 activity (e.g., TWIK-9 gene expression) as identified by a screening assay described herein can be administered to individuals to treat (prophylactically or therapeutically) TWIK-9-associated disorders (e.g., a CNS disorder, a pain disorder, a cardiovascular disorder, or a cell proliferation, growth or differentiation disorder) associated with aberrant or unwanted TWIK-9 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a TWIK-9 molecule or TWIK-9 modulator as well as tailoring the dosage and/or therapeutic regimen of treatment with a TWIK-9 molecule or TWIK-9 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate methyltransferase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug's target is known (e.g., a TWIK-9 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-methyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a TWIK-9 molecule or TWIK-9 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a TWIK-9 molecule or TWIK-9 modulator, such as a modulator identified by one of the exemplary screening assays described herein.

4. Use of TWIK-9 Molecules as Surrogate Markers

The TWIK-9 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the TWIK-9 molecules of the invention may be detected, and may be correlated with one or more biological states in vivo. For example, the TWIK-9 molecules of the invention may serve as surrogate markers for one or more disorders or disease states or for conditions leading up to disease states.

As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the causation of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35:258-264; and James (1994) AIDS Treatment News Archive 209.

The TWIK-9 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a TWIK-9 marker) transcription or expression, the amplified marker may be in a quantity which is more readily detectable than the drug itself. Also, the marker may be more easily detected due to the nature of the marker itself; for example, using the methods described herein, anti-TWIK-9 antibodies may be employed in an immune-based detection system for a TWIK-9 protein marker, or TWIK-9-specific radiolabeled probes may be used to detect a TWIK-9 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90:229-238; Schentag (1999) Am. J. Health-Syst. Pharm. 56 Suppl. 3:S21-S24; and Nicolau (1999) Am. J. Health-Syst. Pharm. 56 Suppl. 3:S16-S20.

The TWIK-9 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12):1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., TWIK-9 protein or RNA) for specific tumor markers in a subject, a drug or course of treatment may be selected that is optimized for the treatment of the specific tumor likely to be present in the subject. Similarly, the presence or absence of a specific sequence mutation in TWIK-9 DNA may correlate TWIK-9 drug response. The use of pharmacogenomic markers therefore permits the application of the most appropriate treatment for each subject without having to administer the therapy.

5. Electronic Apparatus Readable Media and Arrays

Electronic apparatus readable media comprising MAGK sequence information is also provided. As used herein, “MAGK sequence information” refers to any nucleotide and/or amino acid sequence information particular to the MAGK molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said MAGK sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon MAGK sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the MAGK sequence information.

A variety of software programs and formats can be used to store the sequence information on the electronic apparatus readable medium. For example, the sequence information can be represented in a word processing text file, formatted in commercially-available software such as WordPerfect and Microsoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon the MAGK sequence information.

By providing MAGK sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a MAGK-associated disease or disorder or a pre-disposition to a MAGK-associated disease or disorder, wherein the method comprises the steps of determining MAGK sequence information associated with the subject and based on the MAGK sequence information, determining whether the subject has a MAGK-associated disease or disorder or a pre-disposition to a MAGK-associated disease or disorder and/or recommending a particular treatment for the disease, disorder or pre-disease condition.

The present invention further provides in an electronic system and/or in a network, a method for determining whether a subject has a MAGK-associated disease or disorder or a pre-disposition to a disease associated with a MAGK wherein the method comprises the steps of determining MAGK sequence information associated with the subject, and based on the MAGK sequence information, determining whether the subject has a MAGK-associated disease or disorder or a pre-disposition to a MAGK-associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a MAGK-associated disease or disorder or a pre-disposition to a MAGK associated disease or disorder associated with MAGK, said method comprising the steps of receiving MAGK sequence information from the subject and/or information related thereto, receiving phenotypic information associated with the subject, acquiring information from the network corresponding to MAGK and/or a MAGK-associated disease or disorder, and based on one or more of the phenotypic information, the MAGK information (e.g., sequence information and/or information related thereto), and the acquired information, determining whether the subject has a MAGK-associated disease or disorder or a pre-disposition to a MAGK-associated disease or disorder (e.g., a cellular growth or proliferation disease or disorder, for example, cancer). The method may further comprise the step of recommending a particular treatment for the disease, disorder or pre-disease condition.

The present invention also provides a business method for determining whether a subject has a MAGK-associated disease or disorder or a pre-disposition to a MAGK-associated disease or disorder, said method comprising the steps of receiving information related to MAGK (e.g., sequence information and/or information related thereto), receiving phenotypic information associated with the subject, acquiring information from the network related to MAGK and/or related to a MAGK-associated disease or disorder, and based on one or more of the phenotypic information, the MAGK information, and the acquired information, determining whether the subject has a MAGK-associated disease or disorder or a pre-disposition to a MAGK-associated disease or disorder. The method may further comprise the step of recommending a particular treatment for the disease, disorder or pre-disease condition.

The invention also includes an array comprising a MAGK sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be MAGK. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a MAGK-associated disease or disorder, progression of MAGK-associated disease or disorder, and processes, such a cellular transformation associated with the MAGK-associated disease or disorder.

The array is also useful for ascertaining the effect of the expression of a gene on the expression of other genes in the same cell or in different cells (e.g., ascertaining the effect of MAGK expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including MAGK) that could serve as a molecular target for diagnosis or therapeutic intervention.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human TWIK-9 cDNA

In this example, the identification and characterization of the gene encoding human TWIK-9 (clone Fbh56115) is described.

Isolation of the Human TWIK-9 cDNA

The invention is based, at least in part, on the discovery of a human gene encoding a novel member of the TWIK-like family of potassium channels. The entire sequence of human clone Fbh56115 was determined and found to contain an open reading frame termed human “TWIK-9”.

The nucleotide sequence encoding the human TWIK-9 is shown in FIG. 38 and is set forth as SEQ ID NO:40. The protein encoded by this nucleic acid comprises about 374 amino acids and has the amino acid sequence shown in FIG. 38 and set forth as SEQ ID NO:41. The coding region (open reading frame) of SEQ ID NO:40 is set forth as SEQ ID NO:42. Clone Fbh56115, comprising the coding region of human TWIK-9, was deposited with the American Type Culture Collection (ATCC®), 10801 University Boulevard, Manassas, Va. 20110-2209, on ______, and assigned Accession No. ______.

Analysis of the Human TWIK-9 Molecules

The amino acid sequence set forth as SEQ ID NO:41 was searched against the Washington University HMM database (FIG. 40). This search resulted in the identification of the protein of SEQ ID NO:41 as a “TWIK channel” family member, e.g., as having a score of 587.6 when aligned to the TWIK family HMM having PFAM Accession No. PF02034.

Analysis of the amino acid sequence of human TWIK-9 was also performed using MEMSAT. This analysis resulted in the identification of potential transmembrane domains in the amino acid sequence of human TWIK-9 at residues 9-26, 80-96, 108-125, 159-178, 190-208 and 219-243 of SEQ ID NO:41. Further analysis of the TWIK-9 amino acid sequence resulted in a determination that residues 80-96 and 190-208 of SEQ ID NO:41, in fact, correspond to P-loops or pores, as defined herein. Yet further analysis of the TWIK-9 amino acid sequence resulted in a determination that residues 9-26, 108-125, 159-178 and 219-243 correspond to transmembrane domain 1, transmembrane domain 2, transmembrane domain 3 and transmembrane domain 4, respectively. Moreover, amino acid residues 346-362 are predicted not to correspond to a transmembrane domain due, at least in part, to the low score of 0.2

The amino acid sequence of human TWIK-9 was also analyzed using the program PSORT (http://www.psort.nibb.ac.jp) to predict potential localization of the protein within the cell. This program assesses the presence of different targeting and localization amino acid sequences within the query sequence. The results of the analyses show that human TWIK-9 could be localized to the endoplasmic reticulum, if found intracellularly.

A SignalP signal peptide prediction program also recognized the hydrophobic nature of transmembrane domain 1 as a possible signal sequence, e.g., at about amino acids 1-24 of SEQ ID NO:41, although signal and structural analysis as described above indicates more strongly that residues 9-26 are a transmembrane domain.

Searches of the amino acid sequence of human TWIK-9 were further performed against the Prosite database, and resulted in the identification of several possible phosphorylation sites within the amino acid sequence of human TWIK-9 (SEQ ID NO:41). Protein kinase C phosphorylation sites were identified at residues319-321, 331-333 and 341-343; a cAMP and cGMP dependent protein kinase phosphorylation site was identified at residues 370-373; and casein kinase II phosphorylation sites were identified at residues 31-34, 55-58, 127-130, 179-182, 251-254 and 360-363 of human TWIK-9. The search also identified the presence of a N-glycosylation site motif at amino acid residues 53-56; and N-myristoylation site motifs at amino acid residues 102-107, 117-122, 148-153, 236-241 and 266-271 of human TWIK-9.

To further identify potential structural and/or functional properties in a protein of interest, the amino acid sequence of the protein is searched against a database of annotated protein domains (e.g., the ProDom database) using the default parameters (available at http://www.toulouse.inra.fr/prodom.html). A search of the amino acid sequence of human TWIK-9 (SEQ ID NO:41) was performed against the ProDom database. This search resulted in the local alignment of the human TWIK-9 protein with various potassium channel proteins. Specifically, amino acid residues 1-77, 75-212, 78-246, 78-247, 247-327, 288-374 and 328-374 of SEQ ID NO:41 have significant identity to various potassium channel proteins, e.g., various TWIK-related, acid-sensitive, potassium channels and/or other potassium channel subfamily members.

Tissue Distribution of TWIK-9 mRNA

TWIK-9 expression was also determined by PCR analysis of cDNA libraries from various tissues and cell lines. Detecting expression by a library array procedure entailed preparing a PCR mixture including Taq Polymerase, dNTPs, and PCR buffer, and adding a vector primer, a primer internal to the gene of interest, and an aliquot of a library in which expression was to be tested. This procedure was performed with many libraries at a time in a 96 well PCR tray, with 80 or more wells containing libraries and a control well in which the above primers were combined with the clone of interest itself. The control well served as an indicator of the fragment size to be expected in the library wells, in the event the clone of interest was expressed within. Amplification was performed in a PCR machine, employing standard PCR conditions for denaturing, annealing, and elongation, and the resultant mixture was mixed with an appropriate loading dye and run on an ethidium bromide-stained agarose gel. The gel was later viewed with UV light after the DNA loaded within its lanes had time to migrate into the gels. Lanes in which a band corresponding with the control band was visible indicated the libraries in which the clone of interest was expressed.

TWIK-9 was expressed in mouse fetal brain, fetal thymus, cerebellum, dorsal root ganglia, testes, and mammary gland, as well as in Th2 induced T cells and WI-38 cells released from serum starvation.

### Example 2

### Expression of Recombinant TWIK-9 Protein in Bacterial Cells

In this example, human TWIK-9 is expressed as a recombinant glutathione-S-transferase (GST) fusion polypeptide in E. coli and the fusion polypeptide is isolated and characterized. Specifically, human TWIK-9 is fused to GST and this fusion polypeptide is expressed in E. coli, e.g., strain PEB199. Expression of the GST-TWIK-9 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant TWIK-9 Protein in COS Cells

To express the TWIK-9 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire TWIK-9 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the TWIK-9 DNA sequence is amplified by PCR using two primers. The 5′ primer contains the restriction site of interest followed by approximately twenty nucleotides of the TWIK-9 coding sequence starting from the initiation codon; the 3′ end sequence contains complementary sequences to the other restriction site of interest, a translation stop codon, the HA tag or FLAG tag and the last 20 nucleotides of the TWIK-9 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the TWIK-9 gene is inserted in the correct orientation. The ligation mixture is transformed into E. coli cells (strains HB101, DH5□, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the TWIK-9-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2^{nd}, ed., Cold Spring Harbor Laboratory, Cold Spring Harbor Laboratory Press, Cold Spring Harbor, N.Y., 1989. The expression of the TWIK-9 polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the TWIK-9 coding sequence is cloned directly into the polylinker of the pCDNA/Amp vector using the appropriate restriction sites. The resulting plasmid is transfected into COS cells in the manner described above, and the expression of the TWIK-9 polypeptide is detected by radiolabeling and immunoprecipitation using a TWIK-9 specific monoclonal antibody.

### IX. 25658, A NOVEL HUMAN CALCIUM CHANNEL SUBUNIT AND USES THEREOF

### Background of the Invention

Ion channels constitute a large family of membrane-bound proteins responsible for a wide range of important transport and signaling functions in cells. The ion channel family includes at least three subfamilies: calcium ion channels (Ca²⁺ channels), potassium channels (K⁺ channels) and sodium channels (Na⁺ channels). Members of this family regulate ion selectivity in response to a specific stimulus such as a change in voltage across a biological membrane (voltage-gated channels), a mechanical stress (mechanically gated channels, or the binding of a ligand (ligand-gated channels). Gated channels share several features: (1) sensors that are sensitive to chemical or physical signals, (2) gates that open and close in response to the sensors, (3) a pore that selectively permeates ions, and (4) a selectivity filter that permits the channel an ionic discrimination capacity (Triggle (1999) Europ. J. Pharmacol. 375:311-325).

Voltage-gated channel superfamily members are present in the plasma membrane of all electrically excitable cells including neuronal, muscle, endocrine, and egg cells. These channels are responsible for the generation of action potentials that are triggered by depolarization of the plasma membrane (i.e., a shift in the membrane potential to a less negative value). This superfamily includes the voltage-gated Ca²⁺ channels which regulate Ca²⁺ concentrations in a cell in response to depolarization.

The voltage-gated Ca²⁺ channel is a multisubunit complex consisting of at least three different subunits: α₁, β, and α₂δ subunits (Felix et al. (1997) J. Neurosci. 17:6884-6891). The α₁ subunit is the pore forming subunit, while the β and α₂δ subunits are regulatory subunits responsible for current amplitude. Five subclasses of voltage-gated Ca²⁺ channels have been identified based on biophysical and pharmacological properties: T-, N-, P/Q-, R-, and L-types. Each subtype expresses a distinct α₁ subunit and has a selective association with the β, and α₂δ subunits. The P/Q, N, and R subclasses have a neuronal expression, the T subclass has a widespread expression, and the L subclass is expressed in neuroendocrine cells, cardiac cells, smooth muscle cells, and skeletal muscle cells.

Three α₂δ subunits have been identified to date (Klugbauer et al. (1999) J. Neurosci. 19:684-691). The subunits have very little sequence homology, but share several structural characteristics such as containing numerous glycosylation sites and cysteine residues, having similar hydropathy profiles and similar electrophysiological characteristics. α₂δ subunits are expressed in various tissues including heart, pancreas, skeletal muscle, and brain.

Calcium signaling has been implicated in the regulation of a variety of cellular responses, such as growth and differentiation. Voltage-gated Ca²⁺ channels in particular have been associated with the regulation disorders such as angina, atrial fibrillation and flutter, hypertension, peripheral vascular disorders, and cerebral vasospasm (Triggle (1999) Europ. J. Pharmacol. 375:311-325).

### Summary of the Invention

The present invention is based, at least in part, on the discovery of a novel class of alpha-2/delta subunits of the voltage-gated Ca²⁺ channel superfamily, referred to herein as “alpha-2/delta-4” or “α₂δ-4” nucleic acid and polypeptide molecules. The α₂δ-4 nucleic acid and polypeptide molecules of the present invention are useful as modulating agents in regulating a variety of cellular processes, e.g., muscle contraction, membrane excitability, neurite outgrowth and synaptogenesis, signal transduction, cell proliferation, growth, differentiation, and migration, and nociception. Accordingly, in one aspect, this invention provides isolated nucleic acid molecules encoding α₂δ-4 polypeptides or biologically active portions thereof, as well as nucleic acid fragments suitable as primers or hybridization probes for the detection of α₂δ-4-encoding nucleic acids.

In one embodiment, the invention features an isolated nucleic acid molecule that includes the nucleotide sequence set forth in SEQ ID NO:43 or 45. In another embodiment, the invention features an isolated nucleic acid molecule that encodes a polypeptide including the amino acid sequence set forth in SEQ ID NO:44. In another embodiment, the invention features an isolated nucleic acid molecule that includes the nucleotide sequence contained in the plasmid deposited with ATCC® as Accession Number ______.

In still other embodiments, the invention features isolated nucleic acid molecules including nucleotide sequences that are substantially identical (e.g., 60% identical) to the nucleotide sequence set forth as SEQ ID NO:43 or 45. The invention further features isolated nucleic acid molecules including at least 50 contiguous nucleotides of the nucleotide sequence set forth as SEQ ID NO:43 or 45. In another embodiment, the invention features isolated nucleic acid molecules which encode a polypeptide including an amino acid sequence that is substantially identical (e.g., 60% identical) to the amino acid sequence set forth as SEQ ID NO:44. The present invention also features nucleic acid molecules which encode allelic variants of the polypeptide having the amino acid sequence set forth as SEQ ID NO:44. In addition to isolated nucleic acid molecules encoding full-length polypeptides, the present invention also features nucleic acid molecules which encode fragments, for example, biologically active or antigenic fragments, of the full-length polypeptides of the present invention (e.g., fragments including at least 10 contiguous amino acid residues of the amino acid sequence of SEQ ID NO:44). In still other embodiments, the invention features nucleic acid molecules that are complementary to, antisense to, or hybridize under stringent conditions to the isolated nucleic acid molecules described herein.

In another aspect, the invention provides vectors including the isolated nucleic acid molecules described herein (e.g., α₂δ-4-encoding nucleic acid molecules). Such vectors can optionally include nucleotide sequences encoding heterologous polypeptides. Also featured are host cells including such vectors (e.g., host cells including vectors suitable for producing α₂δ-4 nucleic acid molecules and polypeptides).

In another aspect, the invention features isolated α₂δ-4 polypeptides and/or biologically active or antigenic fragments thereof Exemplary embodiments feature a polypeptide including the amino acid sequence set forth as SEQ ID NO:44, a polypeptide including an amino acid sequence at least 60% identical to the amino acid sequence set forth as SEQ ID NO:44, a polypeptide encoded by a nucleic acid molecule including a nucleotide sequence at least 60% identical to the nucleotide sequence set forth as SEQ ID NO:43 or 45. Also featured are fragments of the full-length polypeptides described herein (e.g., fragments including at least 10 contiguous amino acid residues of the sequence set forth as SEQ ID NO:44) as well as allelic variants of the polypeptide having the amino acid sequence set forth as SEQ ID NO:44.

The α₂δ-4 polypeptides and/or biologically active or antigenic fragments thereof, are useful, for example, as reagents or targets in assays applicable to treatment and/or diagnosis of α₂δ-4 mediated or related disorders. In one embodiment, an α₂δ-4 polypeptide or fragment thereof, has an α₂δ-4 activity. In another embodiment, an α₂δ-4 polypeptide or fragment thereof, has a transmembrane domain, and optionally, has an α₂δ-4 activity. In a related aspect, the invention features antibodies (e.g., antibodies which specifically bind to any one of the polypeptides described herein) as well as fusion polypeptides including all or a fragment of a polypeptide described herein.

The present invention further features methods for detecting α₂δ-4 polypeptides and/or α₂δ-4 nucleic acid molecules, such methods featuring, for example, a probe, primer or antibody described herein. Also featured are kits e.g., kits for the detection of α₂δ-4 polypeptides and/or α₂δ-4 nucleic acid molecules. In a related aspect, the invention features methods for identifying compounds which bind to and/or modulate the activity of an α₂δ-4 polypeptide or α₂δ-4 nucleic acid molecule described herein. Further featured are methods for modulating an α₂δ-4 activity.

Other features and advantages of the invention will be apparent from the following detailed description and claims.

### Detailed Description of the Invention

The present invention is based, at least in part, on the discovery of novel molecules, referred to herein as “alpha-2/delta-4” or “α₂δ-4” nucleic acid and polypeptide molecules, which are novel members of the ion channel, e.g., voltage-gated Ca²⁺ channel, family. These novel molecules are capable of, for example, modulating an ion-channel mediated activity (e.g., a calcium channel-mediated activity) in a cell, e.g., a neuronal, muscle (e.g., smooth muscle (e.g., cardiac or vascular) or skeletal muscle), or neuroendocrine cell.

As used herein, an “ion channel” includes a protein or polypeptide which is involved in receiving, conducting, and transmitting signals in an electrically excitable cell, e.g., a neuronal, muscle, or neuroendocrine cell. As used herein, a “voltage-gated calcium channel” includes a protein or polypeptide which is involved in receiving, conducting, and transmitting calcium ion-based signals in an electrically excitable cell. Voltage-gated calcium channels are calcium ion selective, and can determine membrane excitability (the ability of, for example, a muscle cell to contract). Voltage-gated calcium channels can also influence the resting potential of membranes, wave forms and frequencies of action potentials, and thresholds of excitation. Voltage-gated calcium channels are typically expressed in electrically excitable cells, e.g., muscle cells, and may form heteromultimeric structures (e.g., composed of more than one type of subunit). Voltage-gated calcium channels may also be found in non-excitable cells (e.g., adipose cells or liver cells), where they may play a role in, e.g., signal transduction. Examples of voltage-gated calcium channels include the low-voltage-gated channels and the high-voltage-gated channels. Voltage-gated calcium channels are described in, for example, Davila et al. (1999) Annals New York Academy of Sciences 868:102-17 and McEnery, M. W. et al. (1998) J. Bioenergetics and Biomembranes 30(4): 409-418, the contents of which are incorporated herein by reference. As the α₂δ-4 molecules of the present invention are calcium channel subunits capable of modulating ion channel mediated activities (e.g., calcium channel mediated activities), they may be useful for developing novel diagnostic and therapeutic agents for ion channel associated disorders (e.g., calcium channel associated disorders).

As used herein, an “ion channel associated disorder” includes a disorder, disease or condition which is characterized by a misregulation of an ion channel mediated activity. For example, a “voltage-gated calcium channel associated disorder” includes a disorder, disease or condition which is characterized by a misregulation of a voltage-gated calcium channel mediated activity. As used herein, an “α₂δ-4 associated disorder” includes a disorder, disease or condition which is characterized by a misregulation of an α₂δ-4 mediated activity (e.g, modulation of an α₁ subunit activity and/or calcium channel mediated activity). Voltage-gated calcium channel associated disorders include angina, atrial fibrillation and flutter, hypertension, peripheral vascular disorders (e.g., Raynaud's), and cerebral vasospasm.

As used herein, an “ion channel mediated activity” includes an activity which involves a calcium channel, e.g., a voltage-regulated calcium channel, in a cell, e.g., in a neuronal cell, a muscular cell, a neuroendocrine cell, or an egg cell associated with receiving, conducting, and transmitting signals, in, for example, a neuron. Ion channel mediated activities include release of neurotransmitters or second messenger molecules (e.g., dopamine or norepinephrine), from cells, e.g., neurons; modulation of resting potential of membranes, wave forms and frequencies of action potentials, and thresholds of excitation; participation in signal transduction pathways, and modulation of processes such as integration of sub-threshold synaptic responses and the conductance of back-propagating action potentials in, for example, muscle cells (e.g., changes in those action potentials resulting in a morphological or differentiative response in the cell).

As used herein, an “α₂δ-4 mediated activity” includes an activity which involves the regulation of an α₁ and/or any voltage-gated calcium channel activity. α₂δ-4 mediated activities include the interaction with and/or modulation of another non-α₂δ-4 subunit, interaction with and/or modulation of an α₁ calcium channel subunit, modulation of the conductance of an α₁ calcium channel subunit, and interaction with and/or modulation of a non-α₂δ-4 delta subunit.

The term “family” when referring to the polypeptide and nucleic acid molecules of the invention is intended to mean two or more polypeptides or nucleic acid molecules having a common structural domain or motif and having sufficient amino acid or nucleotide sequence homology as defined herein. Such family members can be naturally or non-naturally occurring and can be from either the same or different species. For example, a family can contain a first polypeptide of human origin, as well as other, distinct polypeptides of human origin or alternatively, can contain homologues of non-human origin, e.g., monkey polypeptides. Members of a family may also have common functional characteristics.

For example, the family of α₂δ-4 polypeptides comprise at least one “transmembrane domain” and preferably two transmembrane domains. As used herein, the term “transmembrane domain” includes an amino acid sequence of about 18-45 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, alanines, valines, phenylalanines, prolines or methionines. Transmembrane domains are described in, for example, Zagotta W. N. et al, (1996) Annual Rev. Neurosci. 19: 235-263, the contents of which are incorporated herein by reference. Amino acid residues 422-442, and 1048-1066 of the α₂δ-4 polypeptide comprise transmembrane domains. Accordingly, α₂δ-4 polypeptides having at least 50-60% homology, preferably about 60-70%, more preferably about 70-80%, or about 80-90% homology with a transmembrane domain of human α₂δ-4 are within the scope of the invention.

In a preferred embodiment, the α₂δ-4 molecules of the invention include at least one, preferably two, three, four, five, six, seven, eight, nine, or ten transmembrane domains

In another embodiment, the α₂δ-4 molecules of the invention are identified based on the presence of a Cache domain in the protein or corresponding nucleic acid molecule. As used herein, the term Cache domain includes a protein domain having an amino acid sequence of about 50-150, preferably about 60-100, more preferably about 70-90 amino acid residues, or about 75, 76, 77, 78, 79, 80, 81, 82, 83, 84, 85 amino acids and having a bit score for the alignment of the sequence to the spectrin family (HMM) of at least 20, preferably 20-30, more preferably 40-45, and even more preferably 50-55, or greater. Preferably, a Cache domain is extracellular, even more preferably, a Chache domain has a small molecule recognition and/or signaling activity (e.g. a ligand, agonist or antagonist recognition activity). At least one cache family HMM has been assigned the PFAM Accession PF02743.

To identify the presence of a Cache domain in a α₂δ-4 molecule and/or make the determination that a protein of interest has a particular profile, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF02743 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3)405-420 and a detailed description of HMMs can be found, for example, in Gribskov et al. (1990) Meth. Enzymol. 183:146-159; Gribskov et al.(1987) Proc. Natl. Acad. Sci. USA 84:4355-4358; Krogh et al.(1994) J. Mol. Biol. 235:1501-1531; and Stultz et al.(1993) Protein Sci. 2:305-314, the contents of which are incorporated herein by reference. A search was performed against the HMM database resulting in the identification of Cache domains in the amino acid sequence of SEQ ID NO:44. The results of the search are set forth below.

| Cache: | domain 1 of 2, from 402 to 481: score 39.9, E = 9.4e − 10 | | | |
|---|---|---|---|---|
| | | | *->wTepYvdaastgdlViTvsvPvydrtnetenktnkdngdllGVvgiD | |
| | | | ++P+ d +++l+ Tvs+P y + llG vg+D | |
| | 25658 | 402 | FSLPFSDEM-GDGLIMTVSKPCYF------------GNLLLGIVGVD | 435 |
| | | | vpledLlkltksiklGktGYaFivdnnGkvlaHPnlrpvtkllkdw < -* | |
| | | | v l +l+++ ++ + + Y F++d +G++l HP l ++ l++ + | |
| | 25658 | 436 | VDLAYILEDVTYYQDSLASYTFLIDDKGYTLMHPSLTRPYLLSEPP | 481 |
| Cache: | domain 2 of 2, from 721 to 802: score 10.2, E = 0.2 | | | |
| | | | *->wTePYvdaastgdlViTvsvPvydrtnetenktnkdngdllGVvgiD | |
| | | | +T PY d + + V+T+s + ++t + g V+giD | |
| | 25658 | 721 | LTGPYLDVG-GAGYVVTISHTIHS------SSTQLSSGHTVAVMGID | 760 |
| | | | vpledLlkltksi......klGktGYaFivdnnGkvlaHPnl < -* | |
| | | | +l + k + ++ + +++ G + +Fi+ G+++aHP l | |
| | 25658 | 761 | FTLRYFYKVLMDLlpvcnqDGGNKIRCFIMEDRGYLVAHPTL | 802 |

All amino acids are described using universal single letter abbreviations according to these motifs.

Accordingly, in one embodiment, an α₂δ-4 molecule SEQ ID NO:44 includes a Cache domain at about amino acids 402-481 of SEQ ID NO:44, and/or a Cache domain at about 721-802 of SEQ ID NO:44. Such a cache domain has the amino acid sequence:

FSLPFSDEMGDGLIMTVSKPCYFGNLLLGIVGVDVDLAYILEDV TYYQDSLASYTFLIDDKGYTLMHPSLTRPYLLSEPP

In another embodiment, α₂δ-4 molecules having at least 50-60% homology, preferably about 60-70%, more preferably about 70-80%, or about 80-90% homology with a cache domain of human SEQ ID NO:44 are within the scope of the invention.

In yet another embodiment, the α₂δ-4 molecules of the invention are identified based on the presence of a VWA_{—}4 domain in the protein or corresponding nucleic acid molecule. As used herein, the term VWA_{—}4 domain includes a protein domain having an amino acid sequence of about 100-300, preferably about 150-250, more preferably about 175-225 amino acid residues, or about 150-250 amino acids(e.g., 190, 192, 193, 194 of 195 amino acids) and having a bit score for the alignment of the sequence to the spectrin family (HMM) of at least 4, 5, or 6, or greater. The VWA_{—}4 domain HMM can be built using art recognized Markov Modeling software and known VWA_{—}4 protein sequences. Preferably, a VWA domain has a ligand binding activity.

To identify the presence of a VWA_{—}4 domain in a α₂δ-4 molecule and make the determination that a protein of interest has a particular profile, the amino acid sequence of the protein is searched against a database of HMMs using the appropriate parameters (e.g., a threshold of 5, 8, 10, or 15 bits). A search was performed against a HMM database that contained at least one VWA HMM resulting in the identification of a VWA domain in the amino acid sequence of SEQ ID NO:44. The results of the search are set forth below.

| VWA_4: domain 1 of 1, from 175 to 371: score 6.1, E = 0.12 | | | |
|---|---|---|---|
| | | *-> plDvvfllDgSgSmggnrfekvkefv.klveqid.gdrvglvqFssd | |
| | | + +v++lD +S+++ ++++k++++ +++++d+ d +++++ +++ | |
| 25658 | 175 | SKHIVVILDHGASVTDTQLQIAKDAAqVILSAIDeHDKISVLTVADT | 221 |
| | | vrvefpln..........sqskdallealaslsykglkdyslgggTnlg. | |
| | | vr+ + ++ ++ k ++ + s++ + +T + | |
| 25658 | 222 | VRTCSLDQcyktflspatSETKRKMSTFVSSVKS-------SDSPTQHAv | 264 |
| | | ALqyalenlfsesag.rrgagratkiSnvpkvliliTDGesndg..gddp | |
| | | + q+a++ + +++++ ++ ++ v+i ++ G + ++++d+ | |
| 25658 | 265 | GFQKAFQL-IRSTNNnTKFQANTD------MVIIYLSAGITSKDssEEDK | 307 |
| | | edileaakelkrsg...vkvfvigvgna......de.elkeiasep.geh | |
| | | + +l++++e + +++v++ + ++ n++ ++ + +l+ +a +++g + | |
| 25658 | 308 | KATLQVINEENSFLnnsVMILTYALMNDgvtglkELaFLRDLAEQNsGKY | 357 |
| | | vffdvsdvedlpslldllidlll<-* | |
| | | +p+ ++l + + | |
| 25658 | 358 | G---------VPDRTALPVIKGS | 371 |

Accordingly, in one embodiment, an α₂δ-4 molecule SEQ ID NO:44 which includes a VWA_{—}4 domain at about amino acids 175-371 of SEQ ID NO:44. Such a VWA_{—}4 domain has the amino acid sequence:

SKHIVVILDHGASVTDTQLQIAKDAAQVILSAIDEHDKISVLTVADTVRT
CSLDQCYKTFLSPATSETKRKMSTFVSSVKSSDSPTQHAVGFQKAFQLIR
STNNNTKFQANTDMVIIYLSAGITSKDSSEEDKKATLQVINEENSFLNNS
VMILTYALMNDGVTGLKELAFLRDLAEQNSGKYGVPDRTALPVIKGS

In another embodiment, α₂δ-4 molecules having at least 50-60% homology, preferably about 60-70%, more preferably about 70-80%, or about 80-90% homology with a VWA_{—}4 domain profile of human SEQ ID NO:44 are within the scope of the invention.

Isolated polypeptides of the present invention, preferably α₂δ-4 polypeptides, have an amino acid sequence sufficiently identical to the amino acid sequence of SEQ ID NO:44 or are encoded by a nucleotide sequence sufficiently identical to SEQ ID NO:43 or 45. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains having at least 50%, 55%, 60%, 65%, 70%, 75%, 80%, 85%, 85%, 90% 95%, 96%, 97%, 98%, 99% or more homology or identity across the amino acid sequences of the domains and contain at least one and preferably two structural domains or motifs, are defined herein as sufficiently identical. Furthermore, amino acid or nucleotide sequences which share at least 50%, 55%, 60%, 65%, 70%, 75%, 80%, 85%, 85%, 90%, 95%, 96%, 97%, 98%, 99% or more homology or identity and share a common functional activity are defined herein as sufficiently identical.

In a preferred embodiment, an α₂δ-4 polypeptide includes at least one or more transmembrane domain, and has an amino acid sequence at least about 50%, 55%, 60%, 65%, 70%, 75%, 80%, 85%, 85%, 90%, 95%, 96%, 97%, 98%, 99% or more homologous or identical to the amino acid sequence of SEQ ID NO:44, or the amino acid sequence encoded by the DNA insert of the plasmid deposited with ATCC as Accession Number ______. In yet another preferred embodiment, an α₂δ-4 polypeptide includes at least one or more transmembrane domain, and is encoded by a nucleic acid molecule having a nucleotide sequence which hybridizes under stringent hybridization conditions to a complement of a nucleic acid molecule comprising the nucleotide sequence of SEQ ID NO:43 or 45. In another preferred embodiment, an α₂δ-4 polypeptide includes at least one transmembrane domain, and has an α₂δ-4 activity.

As used interchangeably herein, an “α₂δ-4 activity”, “biological activity of α₂δ-4” or “functional activity of α₂δ-4”, refers to an activity exerted by an α₂δ-4 polypeptide or nucleic acid molecule, for example, an α₂δ-4 expressing cell or tissue, as determined in vivo, or in vitro, according to standard techniques. In one embodiment, an α₂δ-4 activity is a direct activity, such as an association with an α₂δ-4-target molecule. As used herein, a “target molecule” or “binding partner” is a molecule with which an α₂δ-4 polypeptide binds or interacts in nature, such that α₂δ-4-mediated function is achieved. An α₂δ-4 target molecule can be a non-α₂δ-4 molecule or an α₂δ-4 polypeptide or polypeptide of the present invention. In an exemplary embodiment, an α₂δ-4 target molecule another calcium channel subunit, e.g., a calcium channel α subunit. In another exemplary embodiment, an α₂δ-4 target molecule is a calcium channel ligand such as calcium. The biological activities of α₂δ-4 are described herein. For example, the α₂δ-4 polypeptides of the present invention can have one or more of the following activities: (1) interaction with and/or modulation of another non-α₂δ-4 subunit; (2) interaction with and/or modulation of an α₁ calcium channel subunit; (3) modulation of the conductance of an α₁ calcium channel subunit; (4) interaction with and/or modulation of a non-α₂δ-4 delta subunit; (5) modulation of membrane excitability; (6) influencing the resting potential of membranes; (7) modulation of wave forms and frequencies of action potentials; (8) modulation of thresholds of excitation; (9) modulation of neurite outgrowth and synaptogenesis; (10) modulation of signal transduction; and (7) participation in nociception. Alternatively, an α₂δ-4 activity is an indirect activity, such as a cellular signaling activity mediated by interaction of the α₂δ-4 polypeptide with another calcium channel subunit or a calcium channel ligand.

Accordingly, another embodiment of the invention features isolated α₂δ-4 polypeptides and polypeptides having an α₂δ-4 activity. Preferred polypeptides are α₂δ-4 polypeptides having at least one or more transmembrane domain, and preferably, an α₂δ-4 activity.

Additional preferred polypeptides have one or more transmembrane domain, and are, preferably, encoded by a nucleic acid molecule having a nucleotide sequence which hybridizes under stringent hybridization conditions to a complement of a nucleic acid molecule comprising the nucleotide sequence of SEQ ID NO:43 or 45.

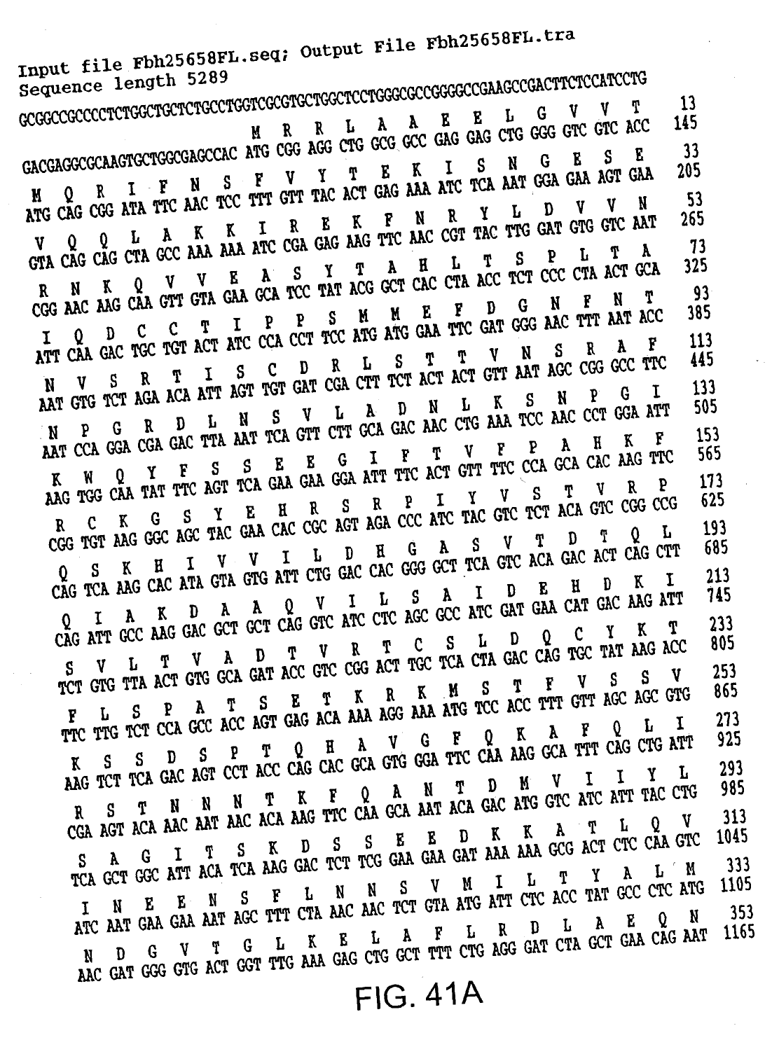

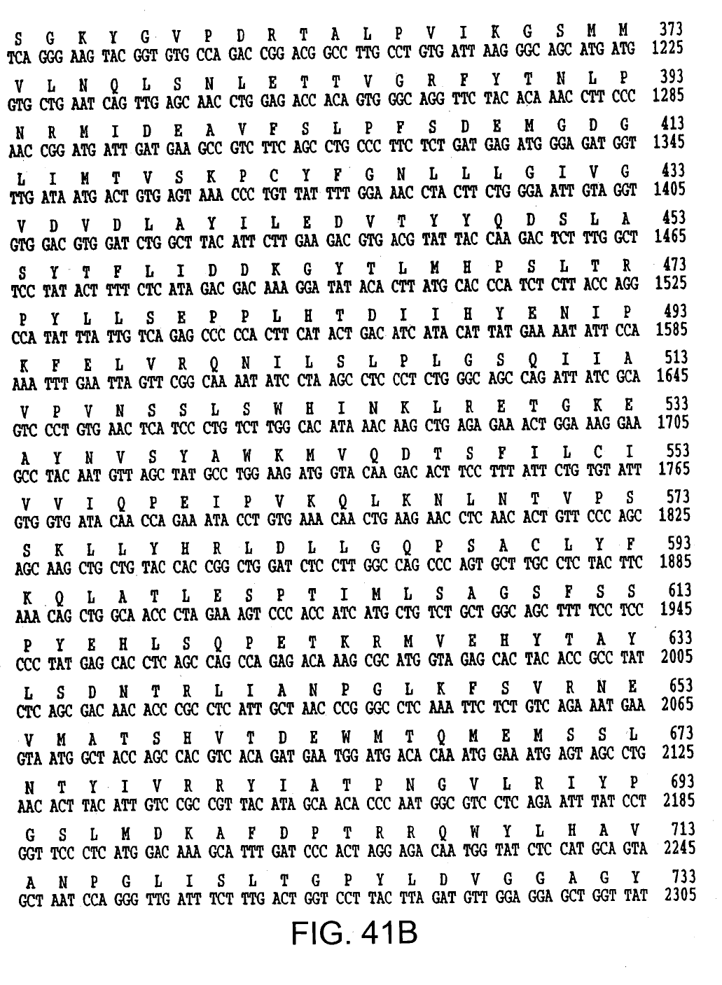

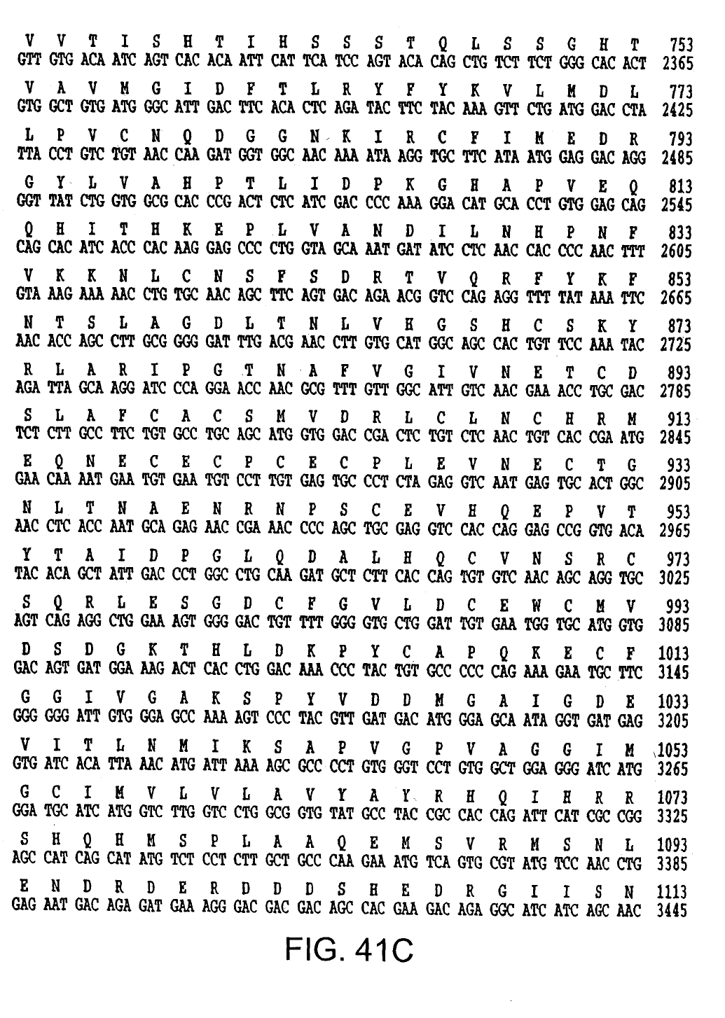

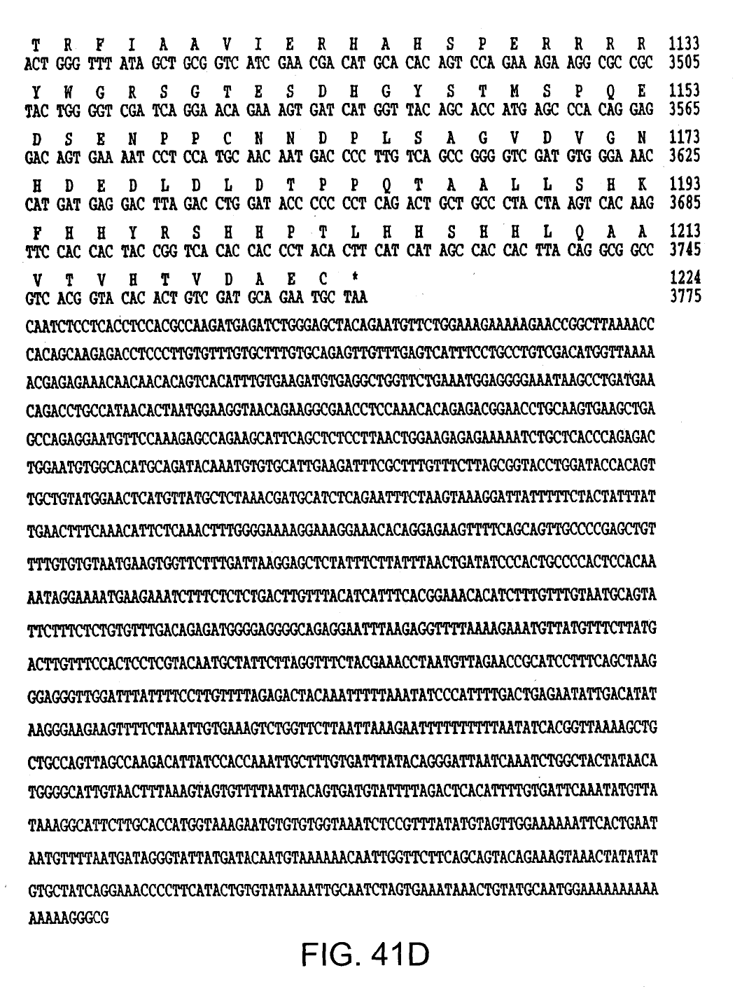

The nucleotide sequence of the isolated human α₂δ-4 cDNA and the predicted amino acid sequence of the human α₂δ-4 polypeptide are shown in FIGS. 41A-D and in SEQ ID NOs:43 and 44, respectively. A plasmid containing the nucleotide sequence encoding human α₂δ-4 was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The human α₂δ-4 gene, which is approximately 5489 nucleotides in length, encodes a polypeptide which is approximately 1223 amino acid residues in length.

Various aspects of the invention are described in further detail in the following subsections:

I. Isolated Nucleic Acid Molecules

One aspect of the invention pertains to isolated nucleic acid molecules that encode α₂δ-4 polypeptides or biologically active portions thereof, as well as nucleic acid fragments sufficient for use as hybridization probes to identify α₂δ-4-encoding nucleic acid molecules (e.g., α₂δ-4 mRNA) and fragments for use as PCR primers for the amplification or mutation of α₂δ-4 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated α₂δ-4 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:43 or 45, or the nucleotide sequence of the DNA insert of the plasmid deposited with ATCC as Accession Number ______, or a portion thereof, can be isolated using standard molecular biology techniques and the sequence information provided herein. Using all or a portion of the nucleic acid sequence of SEQ ID NO:43 or 45, or the nucleotide sequence of the DNA insert of the plasmid deposited with ATCC as Accession Number ______, as a hybridization probe, α₂δ-4 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:43 or 45, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:43 or 45, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to α₂δ-4 nucleotide sequences can be prepared by standard synthetic techniques, e.g., using an automated DNA synthesizer.

In one embodiment, an isolated nucleic acid molecule of the invention comprises the nucleotide sequence shown in SEQ ID NO:43. The sequence of SEQ ID NO:43 corresponds to the human α₂δ-4 cDNA. This cDNA comprises sequences encoding the human α₂δ-4 polypeptide (i.e., “the coding region”, from nucleotides 117-3789) as well as 5′ untranslated sequences (nucleotides 1-116) and 3′ untranslated sequences (nucleotides 3790-5489). Alternatively, the nucleic acid molecule can comprise only the coding region of SEQ ID NO:43 (e.g., nucleotides 117-3789, corresponding to SEQ ID NO:45). Accordingly, in another embodiment, the isolated nucleic acid molecule comprises SEQ ID NO:45 and nucleotides 1-116 and 3790-5489 of SEQ ID NO:43. In yet another embodiment, the nucleic acid molecule consists of the nucleotide sequence set forth as SEQ ID NO:43 or 45.

In still another embodiment, an isolated nucleic acid molecule of the invention comprises a nucleic acid molecule which is a complement of the nucleotide sequence shown in SEQ ID NO:43 or 45, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:43 or 45, or the nucleotide sequence of the DNA insert of the plasmid deposited with ATCC as Accession Number ______, is one which is sufficiently complementary to the nucleotide sequence shown in SEQ ID NO:43 or 45, or the nucleotide sequence of the DNA insert of the plasmid deposited with ATCC as Accession Number ______, such that it can hybridize to the nucleotide sequence shown in SEQ ID NO:43 or 45, or the nucleotide sequence of the DNA insert of the plasmid deposited with ATCC as Accession Number ______, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 96%, 97%, 98%, 99% or more identical to the nucleotide sequence shown in SEQ ID NO:43 or 45 (e.g., to the entire length of the nucleotide sequence), or to the nucleotide sequence (e.g., the entire length of the nucleotide sequence) of the DNA insert of the plasmid deposited with ATCC as Accession Number ______, or a portion of any of these nucleotide sequences. In one embodiment, a nucleic acid molecule of the present invention comprises a nucleotide sequence which is at least (or no greater than) 50-100, 100-250, 250-500, 500-750, 750-1000, 1000-1250, 1250-1500, 1500-1700, 1700-2000, 2000-2500, 2500-3000, 3000-3500, 3500-4000, 4000-4500, 4500-5000 or more nucleotides in length and hybridizes under stringent hybridization conditions to a complement of a nucleic acid molecule of SEQ ID NO:43 or 45, or the nucleotide sequence of the DNA insert of the plasmid deposited with ATCC as Accession Number ______.

Moreover, the nucleic acid molecule of the invention can comprise only a portion of the nucleic acid sequence of SEQ ID NO:43 or 45, or the nucleotide sequence of the DNA insert of the plasmid deposited with ATCC as Accession Number ______, for example, a fragment which can be used as a probe or primer or a fragment encoding a portion of an α₂δ-4 polypeptide, e.g., a biologically active portion of an α₂δ-4 polypeptide. The nucleotide sequence determined from the cloning of the α₂δ-4 gene allows for the generation of probes and primers designed for use in identifying and/or cloning other α₂δ-4 family members, as well as α₂δ-4 homologues from other species. The probe/primer typically comprises substantially purified oligonucleotide. The probe/primer (e.g., oligonucleotide) typically comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95, or 100 or more consecutive nucleotides of a sense sequence of SEQ ID NO:43 or 45, or the nucleotide sequence of the DNA insert of the plasmid deposited with ATCC as Accession Number ______, of an antisense sequence of SEQ ID NO:43 or 45, or the nucleotide sequence of the DNA insert of the plasmid deposited with ATCC as Accession Number ______, or of a naturally occurring allelic variant or mutant of SEQ ID NO:43 or 45, or the nucleotide sequence of the DNA insert of the plasmid deposited with ATCC as Accession Number ______.

Exemplary probes or primers are at least (or no greater than)12 or 15, 20 or 25, 30, 35, 40, 45, 50, 55, 60, 65, 70, 75 or more nucleotides in length and/or comprise consecutive nucleotides of an isolated nucleic acid molecule described herein. Also included within the scope of the present invention are probes or primers comprising contiguous or consecutive nucleotides of an isolated nucleic acid molecule described herein, but for the difference of 1, 2, 3, 4, 5, 6, 7, 8, 9 or 10 bases within the probe or primer sequence. Probes based on the α₂δ-4 nucleotide sequences can be used to detect (e.g., specifically detect) transcripts or genomic sequences encoding the same or homologous polypeptides. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of an α₂δ-4 sequence, e.g., a domain, region, site or other sequence described herein. The primers should be at least 5, 10, or 50 base pairs in length and less than 100, or less than 200, base pairs in length. The primers should be identical, or differs by no greater than 1, 2, 3, 4, 5, 6, 7, 8, 9 or 10 bases when compared to a sequence disclosed herein or to the sequence of a naturally occurring variant. Such probes can be used as a part of a diagnostic test kit for identifying cells or tissue which misexpress an α₂δ-4 polypeptide, such as by measuring a level of an α₂δ-4-encoding nucleic acid in a sample of cells from a subject e.g., detecting α₂δ-4 mRNA levels or determining whether a genomic α₂δ-4 gene has been mutated or deleted.

A nucleic acid fragment encoding a “biologically active portion of an α₂δ-4 polypeptide” can be prepared by isolating a portion of the nucleotide sequence of SEQ ID NO:43 or 45, or the nucleotide sequence of the DNA insert of the plasmid deposited with ATCC as Accession Number ______, which encodes a polypeptide having an α₂δ-4 biological activity (the biological activities of the α₂δ-4 polypeptides are described herein), expressing the encoded portion of the α₂δ-4 polypeptide (e.g., by recombinant expression in vitro) and assessing the activity of the encoded portion of the α₂δ-4 polypeptide. In an exemplary embodiment, the nucleic acid molecule is at least 50-100, 100-250, 250-500, 500-700, 750-1000, 1000-1250, 1250-1500, 1500-1700, 1700-2000, 2000-2500, 2500-3000, 3000-3500, 3500-4000, 4000-4500, 4500-5000 or more nucleotides in length and encodes a polypeptide having an α₂δ-4 activity (as described herein).

The invention further encompasses nucleic acid molecules that differ from the nucleotide sequence shown in SEQ ID NO:43 or 45, or the nucleotide sequence of the DNA insert of the plasmid deposited with ATCC as Accession Number ______. Such differences can be due to due to degeneracy of the genetic code, thus resulting in a nucleic acid which encodes the same α₂δ-4 polypeptides as those encoded by the nucleotide sequence shown in SEQ ID NO:43 or 45, or the nucleotide sequence of the DNA insert of the plasmid deposited with ATCC as Accession Number ______. In another embodiment, an isolated nucleic acid molecule of the invention has a nucleotide sequence encoding a polypeptide having an amino acid sequence which differs by at least 1, but no greater than 5, 10, 20, 50 or 100 amino acid residues from the amino acid sequence shown in SEQ ID NO:44, or the amino acid sequence encoded by the DNA insert of the plasmid deposited with the ATCC as Accession Number ______. In yet another embodiment, the nucleic acid molecule encodes the amino acid sequence of human α₂δ-4. If an alignment is needed for this comparison, the sequences should be aligned for maximum homology.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

Allelic variants result, for example, from DNA sequence polymorphisms within a population (e.g., the human population) that lead to changes in the amino acid sequences of the α₂δ-4 polypeptides. Such genetic polymorphism in the α₂δ-4 genes may exist among individuals within a population due to natural allelic variation. As used herein, the terms “gene” and “recombinant gene” refer to nucleic acid molecules which include an open reading frame encoding an α₂δ-4 polypeptide, preferably a mammalian α₂δ-4 polypeptide, and can further include non-coding regulatory sequences, and introns.

Accordingly, in one embodiment, the invention features isolated nucleic acid molecules which encode a naturally occurring allelic variant of a polypeptide comprising the amino acid sequence of SEQ ID NO:44, or an amino acid sequence encoded by the DNA insert of the plasmid deposited with ATCC as Accession Number ______, wherein the nucleic acid molecule hybridizes to a complement of a nucleic acid molecule comprising SEQ ID NO:43 or 45, for example, under stringent hybridization conditions.

Allelic variants of human α₂δ-4 include both functional and non-functional α₂δ-4 polypeptides. Functional allelic variants are naturally occurring amino acid sequence variants of the human α₂δ-4 polypeptide that maintain the ability to bind an α₂δ-4 ligand or substrate and/or modulate membrane excitability or signal transduction. Functional allelic variants will typically contain only conservative substitution of one or more amino acids of SEQ ID NO:44, or substitution, deletion or insertion of non-critical residues in non-critical regions of the polypeptide.

Non-functional allelic variants are naturally occurring amino acid sequence variants of the human α₂δ-4 polypeptide that do not have the ability to form functional calcium channels or to modulate membrane excitability. Non-functional allelic variants will typically contain a non-conservative substitution, a deletion, or insertion or premature truncation of the amino acid sequence of SEQ ID NO:44, or a substitution, insertion or deletion in critical residues or critical regions.

The present invention further provides non-human and non-murine orthologues (e.g., non-human orthologues of the human α₂δ-4 polypeptide). Orthologues of the human α₂δ-4 polypeptides are polypeptides that are isolated from non-human organisms and possess the same α₂δ-4 ligand binding and/or modulation of membrane excitation mechanisms of the human α₂δ-4 polypeptide. Orthologues of the human α₂δ-4 polypeptide can readily be identified as comprising an amino acid sequence that is substantially identical to SEQ ID NO:44.

Moreover, nucleic acid molecules encoding other α₂δ-4 family members and, thus, which have a nucleotide sequence which differs from the α₂δ-4 sequences of SEQ ID NO:43 or 45, or the nucleotide sequence of the DNA insert of the plasmid deposited with ATCC as Accession Number ______ are intended to be within the scope of the invention. For example, another α₂δ-4 cDNA can be identified based on the nucleotide sequence of human α₂δ-4. Moreover, nucleic acid molecules encoding α₂δ-4 polypeptides from different species, and which, thus, have a nucleotide sequence which differs from the α₂δ-4 sequences of SEQ ID NO:43 or 45, or the nucleotide sequence of the DNA insert of the plasmid deposited with ATCC as Accession Number ______ are intended to be within the scope of the invention. For example, a mouse α₂δ-4 cDNA can be identified based on the nucleotide sequence of a human α₂δ-4.

Nucleic acid molecules corresponding to natural allelic variants and homologues of the α₂δ-4 cDNAs of the invention can be isolated based on their homology to the α₂δ-4 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the α₂δ-4 cDNAs of the invention can further be isolated by mapping to the same chromosome or locus as the α₂δ-4 gene.

Orthologues, homologues and allelic variants can be identified using methods known in the art (e.g., by hybridization to an isolated nucleic acid molecule of the present invention, for example, under stringent hybridization conditions). In one embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:43 or 45, or the nucleotide sequence of the DNA insert of the plasmid deposited with ATCC as Accession Number ______. In other embodiment, the nucleic acid is at least 100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1050, 1050-1070, 1070-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-2000, 2000-2500, 2500-3000, 3000-3500, 3500-4000, 4000-4500, 4500-5000 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g, Church and Gilbert (1984) Proc. Natl. Acad. Sci. USA 81:1991-1995, (or alternatively 0.2×SSC, 1% SDS).

Preferably, an isolated nucleic acid molecule of the invention that hybridizes under stringent conditions to the sequence of SEQ ID NO:43 or 45 and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural polypeptide).

In addition to naturally-occurring allelic variants of the α₂δ-4 sequences that may exist in the population, the skilled artisan will further appreciate that changes can be introduced by mutation into the nucleotide sequences of SEQ ID NO:43 or 45, or the nucleotide sequence of the DNA insert of the plasmid deposited with ATCC as Accession Number ______, thereby leading to changes in the amino acid sequence of the encoded α₂δ-4 polypeptides, without altering the functional ability of the α₂δ-4 polypeptides. For example, nucleotide substitutions leading to amino acid substitutions at “non-essential” amino acid residues can be made in the sequence of SEQ ID NO:43 or 45, or the nucleotide sequence of the DNA insert of the plasmid deposited with ATCC as Accession Number ______. A “non-essential” amino acid residue is a residue that can be altered from the wild-type sequence of α₂δ-4 (e.g., the sequence of SEQ ID NO:44) without altering the biological activity, whereas an “essential” amino acid residue is required for biological activity. For example, amino acid residues that are conserved among the α₂δ-4 polypeptides of the present invention, e.g, those present in a transmembrane domain, are predicted to be particularly unamenable to alteration. Furthermore, additional amino acid residues that are conserved between the α₂δ-4 polypeptides of the present invention and other members of the α₂δ-4 family are not likely to be amenable to alteration.

Accordingly, another aspect of the invention pertains to nucleic acid molecules encoding α₂δ-4 polypeptides that contain changes in amino acid residues that are not essential for activity. Such α₂δ-4 polypeptides differ in amino acid sequence from SEQ ID NO:44, yet retain biological activity. In one embodiment, the isolated nucleic acid molecule comprises a nucleotide sequence encoding a polypeptide, wherein the polypeptide comprises an amino acid sequence at least about 50%, 55%, 60%, 65%, 70%, 75%, 80%, 85%, 90%, 95%, 96%, 97%, 98%, 99% or more identical to SEQ ID NO:44 (e.g., to the entire length of SEQ ID NO:44).

An isolated nucleic acid molecule encoding an α₂δ-4 polypeptide identical to the polypeptide of SEQ ID NO:44, can be created by introducing one or more nucleotide substitutions, additions or deletions into the nucleotide sequence of SEQ ID NO:43 or 45, or the nucleotide sequence of the DNA insert of the plasmid deposited with ATCC as Accession Number ______, such that one or more amino acid substitutions, additions or deletions are introduced into the encoded polypeptide. Mutations can be introduced into SEQ ID NO:43 or 45, or the nucleotide sequence of the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution”is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an α₂δ-4 polypeptide is preferably replaced with another amino acid residue from the same side chain family. Alternatively, in another embodiment, mutations can be introduced randomly along all or part of an α₂δ-4 coding sequence, such as by saturation mutagenesis, and the resultant mutants can be screened for α₂δ-4 biological activity to identify mutants that retain activity. Following mutagenesis of SEQ ID NO:43 or 45, or the nucleotide sequence of the DNA insert of the plasmid deposited with ATCC as Accession Number ______, the encoded polypeptide can be expressed recombinantly and the activity of the polypeptide can be determined.

In a preferred embodiment, a mutant α₂δ-4 polypeptide can be assayed for the ability to (1) interact with and/or modulate another non-α₂δ-4 subunit; (2) interact with and/or modulate an α₁ calcium channel subunit; (3) modulate the conductance of an α₁ calcium channel subunit; (4) interact with and/or modulate a non-α₂δ-4 delta subunit; (5) modulate membrane excitability; (6) influence the resting potential of membranes; (7) modulate wave forms and frequencies of action potentials; (8) modulate thresholds of excitation; (9) modulate of neurite outgrowth and synaptogenesis; (10) modulate signal transduction; and (7) participate in nociception..

In addition to the nucleic acid molecules encoding α₂δ-4 polypeptides described above, another aspect of the invention pertains to isolated nucleic acid molecules which are antisense thereto. In an exemplary embodiment, the invention provides an isolated nucleic acid molecule which is antisense to an α₂δ-4 nucleic acid molecule (e.g., is antisense to the coding strand of an α₂δ-4 nucleic acid molecule). An “antisense” nucleic acid comprises a nucleotide sequence which is complementary to a “sense”nucleic acid encoding a polypeptide, e.g, complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire α₂δ-4 coding strand, or to only a portion thereof. In one embodiment, an antisense nucleic acid molecule is antisense to a “coding region” of the coding strand of a nucleotide sequence encoding α₂δ-4. The term “coding region” refers to the region of the nucleotide sequence comprising codons which are translated into amino acid residues (e.g., the coding region of human α₂δ-4 corresponds to SEQ ID NO:3). In another embodiment, the antisense nucleic acid molecule is antisense to a “noncoding region” of the coding strand of a nucleotide sequence encoding α₂δ-4. The term “noncoding region” refers to 5′ and 3′ sequences which flank the coding region that are not translated into amino acids (i.e., also referred to as 5′ and 3′ untranslated regions).

Given the coding strand sequences encoding α₂δ-4 disclosed herein (e.g., SEQ ID NO:3), antisense nucleic acids of the invention can be designed according to the rules of Watson and Crick base pairing. The antisense nucleic acid molecule can be complementary to the entire coding region of α₂δ-4 mRNA, but more preferably is an oligonucleotide which is antisense to only a portion of the coding or noncoding region of α₂δ-4 mRNA. For example, the antisense oligonucleotide can be complementary to the region surrounding the translation start site of α₂δ-4 mRNA (e.g., between the −10 and +10 regions of the start site of a gene nucleotide sequence). An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an α₂δ-4 polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave α₂δ-4 mRNA transcripts to thereby inhibit translation of α₂δ-4 mRNA. A ribozyme having specificity for an α₂δ-4-encoding nucleic acid can be designed based upon the nucleotide sequence of an α₂δ-4 cDNA disclosed herein (i.e., SEQ ID NO:43 or 45, or the nucleotide sequence of the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in an α₂δ-4-encoding mRNA. See, e.g., Cech et al. U.S. Pat. No. 4,987,071; and Cech et al. U.S. Pat. No. 5,116,742. Alternatively, α₂δ-4 mRNA can be used to select a catalytic RNA having a specific ribonuclease activity from a pool of RNA molecules. See, e.g., Bartel, D. and Szostak, J. W. (1993) Science 261:1411-1418.

Alternatively, α₂δ-4 gene expression can be inhibited by targeting nucleotide sequences complementary to the regulatory region of the α₂δ-4 (e.g., the α₂δ-4 promoter and/or enhancers) to form triple helical structures that prevent transcription of the α₂δ-4 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the α₂δ-4 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of α₂δ-4 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of α₂δ-4 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of α₂δ-4 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of α₂δ-4 nucleic acid molecules can be generated which may combine the advantageous properties of PNA and DNA. Such chimeras allow DNA recognition enzymes, (e.g., RNase H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous α₂δ-4 gene within a cell line or microorganism may be modified by inserting a heterologous DNA regulatory element into the genome of a stable cell line or cloned microorganism such that the inserted regulatory element is operatively linked with the endogenous α₂δ-4 gene. For example, an endogenous α₂δ-4 gene which is normally “transcriptionally silent”, i.e., an α₂δ-4 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous α₂δ-4 gene may be activated by insertion of a promiscuous regulatory element that works across cell types.

A heterologous regulatory element may be inserted into a stable cell line or cloned microorganism, such that it is operatively linked with an endogenous α₂δ-4 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated α₂δ-4 Polypeptides and Anti-α₂δ-4 Antibodies

One aspect of the invention pertains to isolated α₂δ-4 or recombinant polypeptides and polypeptides, and biologically active portions thereof, as well as polypeptide fragments suitable for use as immunogens to raise anti-α₂δ-4 antibodies. In one embodiment, native α₂δ-4 polypeptides can be isolated from cells or tissue sources by an appropriate purification scheme using standard protein purification techniques. In another embodiment, α₂δ-4 polypeptides are produced by recombinant DNA techniques. Alternative to recombinant expression, an α₂δ-4 polypeptide or polypeptide can be synthesized chemically using standard peptide synthesis techniques.

An “isolated” or “purified” polypeptide or biologically active portion thereof is substantially free of cellular material or other contaminating proteins from the cell or tissue source from which the α₂δ-4 polypeptide is derived, or substantially free from chemical precursors or other chemicals when chemically synthesized. The language “substantially free of cellular material” includes preparations of α₂δ-4 polypeptide in which the polypeptide is separated from cellular components of the cells from which it is isolated or recombinantly produced. In one embodiment, the language “substantially free of cellular material” includes preparations of α₂δ-4 polypeptide having less than about 30% (by dry weight) of non-α₂δ-4 polypeptide (also referred to herein as a “contaminating protein”), more preferably less than about 20% of non-α₂δ-4 polypeptide, still more preferably less than about 10% of non-α₂δ-4 polypeptide, and most preferably less than about 5% non-α₂δ-4 polypeptide. When the α₂δ-4 polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of α₂δ-4 polypeptide in which the polypeptide is separated from chemical precursors or other chemicals which are involved in the synthesis of the polypeptide. In one embodiment, the language “substantially free of chemical precursors or other chemicals” includes preparations of α₂δ-4 polypeptide having less than about 30% (by dry weight) of chemical precursors or non-α₂δ-4 chemicals, more preferably less than about 20% chemical precursors or non-α₂δ-4 chemicals, still more preferably less than about 10% chemical precursors or non-α₂δ-4 chemicals, and most preferably less than about 5% chemical precursors or non-α₂δ-4 chemicals.

As used herein, a “biologically active portion” of an α₂δ-4 polypeptide includes a fragment of an α₂δ-4 polypeptide which participates in an interaction between an α₂δ-4 molecule and a non-α₂δ-4 molecule. Biologically active portions of an α₂δ-4 polypeptide include peptides comprising amino acid sequences sufficiently identical to or derived from the amino acid sequence of the α₂δ-4 polypeptide, e.g., the amino acid sequence shown in SEQ ID NO:44, which include less amino acids than the full length α₂δ-4 polypeptides, and exhibit at least one activity of an α₂δ-4 polypeptide. Typically, biologically active portions comprise a domain or motif with at least one activity of the α₂δ-4 polypeptide, e.g., modulating membrane excitation mechanisms. A biologically active portion of an α₂δ-4 polypeptide can be a polypeptide which is, for example, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200 or more amino acids in length. Biologically active portions of an α₂δ-4 polypeptide can be used as targets for developing agents which modulate an α₂δ-4 mediated activity, e.g., a membrane excitation mechanism.

In one embodiment, a biologically active portion of an α₂δ-4 polypeptide comprises at least one transmembrane domain. It is to be understood that a preferred biologically active portion of an α₂δ-4 polypeptide of the present invention comprises at least one or more a transmembrane domain. Moreover, other biologically active portions, in which other regions of the polypeptide are deleted, can be prepared by recombinant techniques and evaluated for one or more of the functional activities of a native α₂δ-4 polypeptide.

Another aspect of the invention features fragments of the polypeptide having the amino acid sequence of SEQ ID NO:44, for example, for use as immunogens. In one embodiment, a fragment comprises at least 5 amino acids (e.g., contiguous or consecutive amino acids) of the amino acid sequence of SEQ ID NO:44, or an amino acid sequence encoded by the DNA insert of the plasmid deposited with the ATCC as Accession Number ______. In another embodiment, a fragment comprises at least 10, 15, 20, 25, 30, 35, 40, 45, 50 or more amino acids (e.g., contiguous or consecutive amino acids) of the amino acid sequence of SEQ ID NO:44, or an amino acid sequence encoded by the DNA insert of the plasmid deposited with the ATCC as Accession Number ______.

In a preferred embodiment, an α₂δ-4 polypeptide has an amino acid sequence shown in SEQ ID NO:44. In other embodiments, the α₂δ-4 polypeptide is substantially identical to SEQ ID NO:44, and retains the functional activity of the polypeptide of SEQ ID NO:44, yet differs in amino acid sequence due to natural allelic variation or mutagenesis, as described in detail in subsection I above. In another embodiment, the α₂δ-4 polypeptide is a polypeptide which comprises an amino acid sequence at least about 50%, 55%, 60%, 65%, 70%, 75%, 80%, 85%, 90%, 95%, 96%, 97%, 98%, 99% or more identical to SEQ ID NO:44.

In another embodiment, the invention features an α₂δ-4 polypeptide which is encoded by a nucleic acid molecule consisting of a nucleotide sequence at least about 50%, 55%, 60%, 65%, 70%, 75%, 80%, 85%, 90%, 95%, 96%, 97%, 98%, 99% or more identical to a nucleotide sequence of SEQ ID NO:43 or 45, or a complement thereof. This invention further features an α₂δ-4 polypeptide which is encoded by a nucleic acid molecule consisting of a nucleotide sequence which hybridizes under stringent hybridization conditions to a complement of a nucleic acid molecule comprising the nucleotide sequence of SEQ ID NO:43 or 45, or a complement thereof.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the α₂δ-4 amino acid sequence of SEQ ID NO:44 having 1223 amino acid residues, at least 366, preferably at least 489, more preferably at least 611, more preferably at least 733, even more preferably at least 856, and even more preferably at least 978 or 1100 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of E. Meyers and W. Miller (Comput. Appl. Biosci., 4:11-17 (1988)) which has been incorporated into the ALIGN program (version 2.0 or version 2.0U), using a PAM120 weight residue table, a gap length penalty of 12 and a gap penalty of 4.

The nucleic acid and polypeptide sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to α₂δ-4 nucleic acid molecules of the invention. BLAST protein searches can be performed with the XBLAST program, score=100, wordlength=3, and a Blosum62 matrix to obtain amino acid sequences homologous to α₂δ-4 polypeptide molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides α₂δ-4 chimeric or fusion proteins. As used herein, an α₂δ-4 “chimeric protein” or “fusion protein” comprises an α₂δ-4 polypeptide operatively linked to a non-α₂δ-4 polypeptide. A “α₂δ-4 polypeptide” refers to a polypeptide having an amino acid sequence corresponding to α₂δ-4, whereas a “non-α₂δ-4 polypeptide” refers to a polypeptide having an amino acid sequence corresponding to a polypeptide which is not substantially homologous to the α₂δ-4 polypeptide, e.g., a polypeptide which is different from the α₂δ-4 polypeptide and which is derived from the same or a different organism. Within an α₂δ-4 fusion protein the α₂δ-4 polypeptide can correspond to all or a portion of an α₂δ-4 polypeptide. In a preferred embodiment, an α₂δ-4 fusion protein comprises at least one biologically active portion of an α₂δ-4 polypeptide. In another preferred embodiment, an α₂δ-4 fusion protein comprises at least two biologically active portions of an α₂δ-4 polypeptide. Within the fusion protein, the term “operatively linked” is intended to indicate that the α₂δ-4 polypeptide and the non-α₂δ-4 polypeptide are fused in-frame to each other. The non-α₂δ-4 polypeptide can be fused to the N-terminus or C-terminus of the α₂δ-4 polypeptide.

For example, in one embodiment, the fusion protein is a GST-α₂δ-4 fusion protein in which the α₂δ-4 sequences are fused to the C-terminus of the GST sequences. Such fusion proteins can facilitate the purification of recombinant α₂δ-4.

In another embodiment, the fusion protein is an α₂δ-4 polypeptide containing a heterologous signal sequence at its N-terminus. In certain host cells (e.g., mammalian host cells), expression and/or secretion of α₂δ-4 can be increased through the use of a heterologous signal sequence.

The α₂δ-4 fusion proteins of the invention can be incorporated into pharmaceutical compositions and administered to a subject in vivo. The α₂δ-4 fusion proteins can be used to affect the bioavailability of an α₂δ-4 substrate. Use of α₂δ-4 fusion proteins may be useful therapeutically for the treatment of disorders caused by, for example, (i) aberrant modification or mutation of a gene encoding an α₂δ-4 polypeptide; (ii) mis-regulation of the α₂δ-4 gene; and (iii) aberrant post-translational modification of an α₂δ-4 polypeptide.

Moreover, the α₂δ-4-fusion proteins of the invention can be used as immunogens to produce anti-α₂δ-4 antibodies in a subject, to purify α₂δ-4 ligands and in screening assays to identify molecules which inhibit the interaction of α₂δ-4 with an α₂δ-4 substrate.

Preferably, an α₂δ-4 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g, a GST polypeptide). An α₂δ-4-encoding nucleic acid can be cloned into such an expression vector such that the fusion moiety is linked in-frame to the α₂δ-4 polypeptide.

The present invention also pertains to variants of the α₂δ-4 polypeptides which function as either α₂δ-4 agonists (mimetics) or as α₂δ-4 antagonists. Variants of the α₂δ-4 polypeptides can be generated by mutagenesis, e.g., discrete point mutation or truncation of an α₂δ-4 polypeptide. An agonist of the α₂δ-4 polypeptides can retain substantially the same, or a subset, of the biological activities of the naturally occurring form of an α₂δ-4 polypeptide. An antagonist of an α₂δ-4 polypeptide can inhibit one or more of the activities of the naturally occurring form of the α₂δ-4 polypeptide by, for example, competitively modulating an α₂δ-4-mediated activity of an α₂δ-4 polypeptide. Thus, specific biological effects can be elicited by treatment with a variant of limited function. In one embodiment, treatment of a subject with a variant having a subset of the biological activities of the naturally occurring form of the polypeptide has fewer side effects in a subject relative to treatment with the naturally occurring form of the α₂δ-4 polypeptide.

In one embodiment, variants of an α₂δ-4 polypeptide which function as either α₂δ-4 agonists (mimetics) or as α₂δ-4 antagonists can be identified by screening combinatorial libraries of mutants, e.g., truncation mutants, of an α₂δ-4 polypeptide for α₂δ-4 polypeptide agonist or antagonist activity. In one embodiment, a variegated library of α₂δ-4 variants is generated by combinatorial mutagenesis at the nucleic acid level and is encoded by a variegated gene library. A variegated library of α₂δ-4 variants can be produced by, for example, enzymatically ligating a mixture of synthetic oligonucleotides into gene sequences such that a degenerate set of potential α₂δ-4 sequences is expressible as individual polypeptides, or alternatively, as a set of larger fusion proteins (e.g., for phage display) containing the set of α₂δ-4 sequences therein. There are a variety of methods which can be used to produce libraries of potential α₂δ-4 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential α₂δ-4 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of an α₂δ-4 polypeptide coding sequence can be used to generate a variegated population of α₂δ-4 fragments for screening and subsequent selection of variants of an α₂δ-4 polypeptide. In one embodiment, a library of coding sequence fragments can be generated by treating a double stranded PCR fragment of an α₂δ-4 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the α₂δ-4 polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of α₂δ-4 polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify α₂δ-4 variants (Arkin and Youvan (1992) Proc. Natl. Acad. Sci. USA 89:7811-7815; Delagrave et al. (1993) Protein Engineering 6(3):327-331).

In one embodiment, cell based assays can be exploited to analyze a variegated α₂δ-4 library. For example, a library of expression vectors can be transfected into a cell line, e.g., an endothelial cell line, which ordinarily responds to voltage regulation in a particular voltage-gated calcium channel substrate-dependent manner. The transfected cells are then contacted with α₂δ-4 and the effect of expression of the mutant on signaling by the voltage-gated calcium channel substrate can be detected, e.g., by monitoring intracellular calcium, IP3, or diacylglycerol concentration, phosphorylation profile of intracellular proteins, or the activity of a voltage-gated calcium channel-regulated transcription factor. Plasmid DNA can then be recovered from the cells which score for inhibition, or alternatively, potentiation of signaling by the voltage-gated calcium channel substrate, and the individual clones further characterized.

An isolated α₂δ-4 polypeptide, or a portion or fragment thereof, can be used as an immunogen to generate antibodies that bind α₂δ-4 using standard techniques for polyclonal and monoclonal antibody preparation. A full-length α₂δ-4 polypeptide can be used or, alternatively, the invention provides antigenic peptide fragments of α₂δ-4 for use as immunogens. The antigenic peptide of α₂δ-4 comprises at least 8 amino acid residues of the amino acid sequence shown in SEQ ID NO:44 and encompasses an epitope of α₂δ-4 such that an antibody raised against the peptide forms a specific immune complex with α₂δ-4. Preferably, the antigenic peptide comprises at least 10 amino acid residues, more preferably at least 15 amino acid residues, even more preferably at least 20 amino acid residues, and most preferably at least 30 amino acid residues.

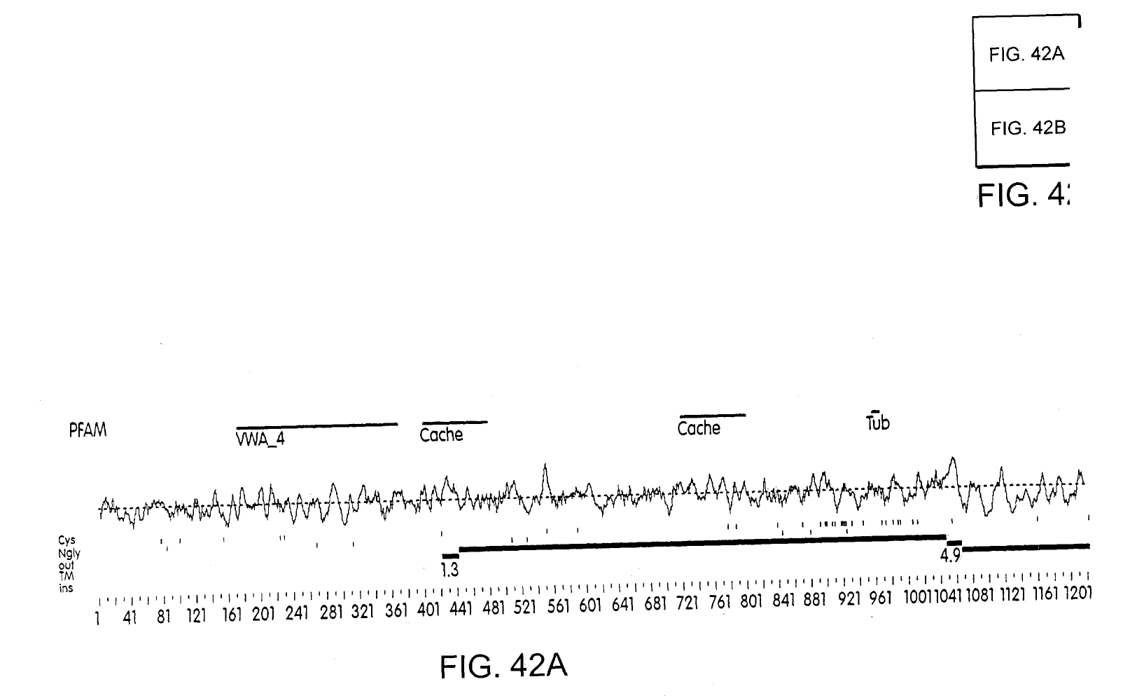

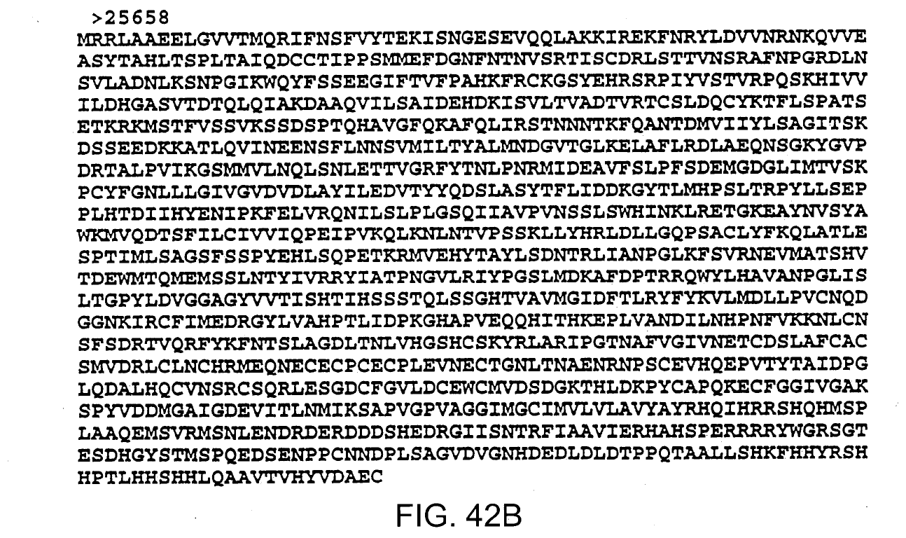

Preferred epitopes encompassed by the antigenic peptide are regions of α₂δ-4 that are located on the surface of the polypeptide, e.g., hydrophilic regions, as well as regions with high antigenicity (see, for example, FIG. 42).

An α₂δ-4 immunogen typically is used to prepare antibodies by immunizing a suitable subject, (e.g., rabbit, goat, mouse or other mammal) with the immunogen. An appropriate immunogenic preparation can contain, for example, recombinantly expressed α₂δ-4 polypeptide or a chemically synthesized α₂δ-4 polypeptide. The preparation can further include an adjuvant, such as Freund's complete or incomplete adjuvant, or similar immunostimulatory agent. Immunization of a suitable subject with an immunogenic α₂δ-4 preparation induces a polyclonal anti-α₂δ-4 antibody response.

Accordingly, another aspect of the invention pertains to anti-α₂δ-4 antibodies. The term “antibody” as used herein refers to immunoglobulin molecules and immunologically active portions of immunoglobulin molecules, i.e., molecules that contain an antigen binding site which specifically binds (immunoreacts with) an antigen, such as α₂δ-4. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind α₂δ-4. The term “monoclonal antibody” or “monoclonal antibody composition”, as used herein, refers to a population of antibody molecules that contain only one species of an antigen binding site capable of immunoreacting with a particular epitope of α₂δ-4. A monoclonal antibody composition thus typically displays a single binding affinity for a particular α₂δ-4 polypeptide with which it immunoreacts.

Polyclonal anti-α₂δ-4 antibodies can be prepared as described above by immunizing a suitable subject with an α₂δ-4 immunogen. The anti-α₂δ-4 antibody titer in the immunized subject can be monitored over time by standard techniques, such as with an enzyme linked immunosorbent assay (ELISA) using immobilized α₂δ-4. If desired, the antibody molecules directed against α₂δ-4 can be isolated from the mammal (e.g., from the blood) and further purified by well known techniques, such as protein A chromatography to obtain the IgG fraction. At an appropriate time after immunization, e.g., when the anti-α₂δ-4 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem .255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an α₂δ-4 immunogen as described above, and the culture supernatants of the resulting hybridoma cells are screened to identify a hybridoma producing a monoclonal antibody that binds α₂δ-4.

Any of the many well known protocols used for fusing lymphocytes and immortalized cell lines can be applied for the purpose of generating an anti-α₂δ-4 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind α₂δ-4, e.g., using a standard ELISA assay.

Alternative to preparing monoclonal antibody-secreting hybridomas, a monoclonal anti-α₂δ-4 antibody can be identified and isolated by screening a recombinant combinatorial immunoglobulin library (e.g., an antibody phage display library) with α₂δ-4 to thereby isolate immunoglobulin library members that bind α₂δ-4. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-α₂δ-4 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-α₂δ-4 antibody (e.g, monoclonal antibody) can be used to isolate α₂δ-4 by standard techniques, such as affinity chromatography or immunoprecipitation. An anti-α₂δ-4 antibody can facilitate the purification of natural α₂δ-4 from cells and of recombinantly produced α₂δ-4 expressed in host cells. Moreover, an anti-α₂δ-4 antibody can be used to detect α₂δ-4 polypeptide (e.g., in a cellular lysate or cell supernatant) in order to evaluate the abundance and pattern of expression of the α₂δ-4 polypeptide. Anti-α₂δ-4 antibodies can be used diagnostically to monitor polypeptide levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, for example recombinant expression vectors, containing a nucleic acid containing an α₂δ-4 nucleic acid molecule or vectors containing a nucleic acid molecule which encodes an α₂δ-4 polypeptide (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, and the like. The expression vectors of the invention can be introduced into host cells to thereby produce proteins or peptides, including fusion proteins or peptides, encoded by nucleic acids as described herein (e.g., α₂δ-4 polypeptides, mutant forms of α₂δ-4 polypeptides, fusion proteins, and the like).

Accordingly, an exemplary embodiment provides a method for producing a polypeptide, preferably an α₂δ-4 polypeptide, by culturing in a suitable medium a host cell of the invention (e.g., a mammalian host cell such as a non-human mammalian cell) containing a recombinant expression vector, such that the polypeptide is produced.

The recombinant expression vectors of the invention can be designed for expression of α₂δ-4 polypeptides in prokaryotic or eukaryotic cells. For example, α₂δ-4 polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in α₂δ-4 activity assays, (e.g., direct assays or competitive assays described in detail below), or to generate antibodies specific for α₂δ-4 polypeptides, for example. In a preferred embodiment, an α₂δ-4 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the α₂δ-4 expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al, (1987) Gene 54:113-123), pYES2 (Invitrogen Corporation, San Diego, Calif.), and picZ (Invitrogen Corporation, San Diego, Calif.).

Alternatively, α₂δ-4 polypeptides can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to α₂δ-4 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which an α₂δ-4 nucleic acid molecule of the invention is introduced, e.g., an α₂δ-4 nucleic acid molecule within a vector (e.g., a recombinant expression vector) or an α₂δ-4 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an α₂δ-4 polypeptide can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an α₂δ-4 polypeptide or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an α₂δ-4 polypeptide. Accordingly, the invention further provides methods for producing an α₂δ-4 polypeptide using the host cells of the invention. In one embodiment, the method comprises culturing the host cell of the invention (into which a recombinant expression vector encoding an α₂δ-4 polypeptide has been introduced) in a suitable medium such that an α₂δ-4 polypeptide is produced. In another embodiment, the method further comprises isolating an α₂δ-4 polypeptide from the medium or the host cell.

The host cells of the invention can also be used to produce non-human transgenic animals. For example, in one embodiment, a host cell of the invention is a fertilized oocyte or an embryonic stem cell into which α₂δ-4-coding sequences have been introduced. Such host cells can then be used to create non-human transgenic animals in which exogenous α₂δ-4 sequence have been introduced into their genome or homologous recombinant animals in which endogenous α₂δ-4 sequences have been altered. Such animals are useful for studying the function and/or activity of an α₂δ-4 and for identifying and/or evaluating modulators of α₂δ-4 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous α₂δ-4 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an α₂δ-4-encoding nucleic acid into the male pronuclei of a fertilized oocyte, e.g., by microinjection, retroviral infection, and allowing the oocyte to develop in a pseudopregnant female foster animal. The α₂δ-4 cDNA sequence of SEQ ID NO:1 can be introduced as a transgene into the genome of a non-human animal. Alternatively, a nonhuman homologue of a human α₂δ-4 gene, such as a mouse or rat α₂δ-4 gene, can be used as a transgene. Alternatively, an α₂δ-4 gene homologue, such as another α₂δ-4 family member, can be isolated based on hybridization to the α₂δ-4 cDNA sequences of SEQ ID NO:43 or 45, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to an α₂δ-4 transgene to direct expression of an α₂δ-4 polypeptide to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of an α₂δ-4 transgene in its genome and/or expression of α₂δ-4 mRNA in tissues or cells of the animals. A transgenic founder animal can then be used to breed additional animals carrying the transgene. Moreover, transgenic animals carrying a transgene encoding an α₂δ-4 polypeptide can further be bred to other transgenic animals carrying other transgenes.

To create a homologous recombinant animal, a vector is prepared which contains at least a portion of an α₂δ-4 gene into which a deletion, addition or substitution has been introduced to thereby alter, e.g., functionally disrupt, the α₂δ-4 gene. The α₂δ-4 gene can be a human gene (e.g., the cDNA of SEQ ID NO:3), but more preferably, is a non-human homologue of a human α₂δ-4 gene (e.g., a cDNA isolated by stringent hybridization with the nucleotide sequence of SEQ ID NO:1). For example, a mouse α₂δ-4 gene can be used to construct a homologous recombination nucleic acid molecule, e.g., a vector, suitable for altering an endogenous α₂δ-4 gene in the mouse genome. In a preferred embodiment, the homologous recombination nucleic acid molecule is designed such that, upon homologous recombination, the endogenous α₂δ-4 gene is functionally disrupted (i.e., no longer encodes a functional protein; also referred to as a “knock out” vector). Alternatively, the homologous recombination nucleic acid molecule can be designed such that, upon homologous recombination, the endogenous α₂δ-4 gene is mutated or otherwise altered but still encodes functional polypeptide (e.g., the upstream regulatory region can be altered to thereby alter the expression of the endogenous α₂δ-4 polypeptide). In the homologous recombination nucleic acid molecule, the altered portion of the α₂δ-4 gene is flanked at its 5′ and 3′ ends by additional nucleic acid sequence of the α₂δ-4 gene to allow for homologous recombination to occur between the exogenous α₂δ-4 gene carried by the homologous recombination nucleic acid molecule and an endogenous α₂δ-4 gene in a cell, e.g., an embryonic stem cell. The additional flanking α₂δ-4 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced α₂δ-4 gene has homologously recombined with the endogenous α₂δ-4 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The α₂δ-4 nucleic acid molecules, fragments of α₂δ-4 polypeptides, and anti-α₂δ-4 antibodies (also referred to herein as “active compounds”) of the invention can be incorporated into pharmaceutical compositions suitable for administration. Such compositions typically comprise the nucleic acid molecule, polypeptide, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of an α₂δ-4 polypeptide or an anti-α₂δ-4 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a polypeptide or antibody can include a single treatment or, preferably, can include a series of treatments.

In a preferred example, a subject is treated with antibody or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, an α₂δ-4 polypeptide of the invention has one or more of the following activities: (1) interaction with and/or modulation of another non-α₂δ-4 subunit; (2) interaction with and/or modulation of an α₁ calcium channel subunit; (3) modulation of the conductance of an α₁ calcium channel subunit; (4) interaction with and/or modulation of a non-α₂δ-4 delta subunit; (5) modulation of membrane excitability; (6) influencing the resting potential of membranes; (7) modulation of wave forms and frequencies of action potentials; (8) modulation of thresholds of excitation; (9) modulation of neurite outgrowth and synaptogenesis; (10) modulation of signal transduction; and (7) participation in nociception.

The isolated nucleic acid molecules of the invention can be used, for example, to express α₂δ-4 polypeptide (e.g., via a recombinant expression vector in a host cell in gene therapy applications), to detect α₂δ-4 mRNA (e.g., in a biological sample) or a genetic alteration in an α₂δ-4 gene, and to modulate α₂δ-4 activity, as described further below. The α₂δ-4 polypeptides can be used to treat disorders characterized by insufficient or excessive production of an α₂δ-4 substrate or production of α₂δ-4 inhibitors. In addition, the α₂δ-4 polypeptides can be used to screen for naturally occurring α₂δ-4 substrates, to screen for drugs or compounds which modulate α₂δ-4 activity, as well as to treat disorders characterized by insufficient or excessive production of α₂δ-4 polypeptide or production of α₂δ-4 polypeptide forms which have decreased, aberrant or unwanted activity compared to α₂δ-4 wild type polypeptide. Moreover, the anti-α₂δ-4 antibodies of the invention can be used to detect and isolate α₂δ-4 polypeptides, to regulate the bioavailability of α₂δ-4 polypeptides, and modulate α₂δ-4 activity.

Examples of ion channel associated disorders include CNS disorders, such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, Creutzfeldt-Jakob disease, or AIDS related dementia; autonomic function disorders such as hypertension and sleep disorders, and neuropsychiatric disorders, such as depression, schizophrenia, schizoaffective disorder, korsakoff's psychosis, mania, anxiety disorders, or phobic disorders; leaning or memory disorders, e.g., amnesia or age-related memory loss, attention deficit disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Ion channel associated disorders include pain disorders. Pain disorders include those that affect pain signaling mechanisms. As used herein, the term “pain signaling mechanisms” includes the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter sensory neurons. These sensory neurons transmit the information to the central nervous system, evoking a perception of pain or discomfort and initiating appropriate protective reflexes. The α₂δ-4 molecules of the present invention may be present on these sensory neurons and, thus, may be involved in detecting these noxious chemical, mechanical, or thermal stimuli and transducing this information into membrane depolarization events. Thus, the α₂δ-4 molecules by participating in pain signaling mechanisms, may modulate pain elicitation and act as targets for developing novel diagnostic targets and therapeutic agents to control pain.

Ion channel associated disorders include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The α₂δ-4 molecules of the present invention are involved in signal transduction mechanisms, which are known to be involved in cellular growth, differentiation, and migration processes. Thus, the α₂δ-4 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; hepatic disorders; cardiovascular disorders; and hematopoietic and/or myeloproliferative disorders.

Preferred α₂δ-4 associated disorders are those associated with aberrant voltage-gated calcium channel activity. Examples of such disorders include angina, atrial fibrillation and flutter, hypertension, peripheral vascular disorders (e.g., Raynaud's), and cerebral vasospasm. Other examples of α₂δ-4 associated disorders are described herein. In addition, the α₂δ-4 polypeptides can be used to screen for naturally occurring α₂δ-4 substrates, to screen for drugs or compounds which modulate α₂δ-4 activity, as well as to treat disorders characterized by insufficient or excessive production of α₂δ-4 polypeptide or production of α₂δ-4 polypeptide forms which have decreased, aberrant or unwanted activity compared to α₂δ-4 wild type polypeptide (such as cell permeabilization, cell necrosis or apoptosis, triggering of second messengers, cell proliferation, cell motility, or signal transduction disorders). Moreover, the anti-α₂δ-4 antibodies of the invention can be used to detect and isolate α₂δ-4 polypeptides, to regulate the bioavailability of α₂δ-4 polypeptides, and modulate α₂δ-4 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to α₂δ-4 polypeptides, have a stimulatory or inhibitory effect on, for example, α₂δ-4 expression or α₂δ-4 activity, or have a stimulatory or inhibitory effect on, for example, the expression or activity of α₂δ-4 substrate.

In one embodiment, the invention provides assays for screening candidate or test compounds which are substrates of an α₂δ-4 polypeptide or polypeptide or biologically active portion thereof. In another embodiment, the invention provides assays for screening candidate or test compounds which bind to or modulate the activity of an α₂δ-4 polypeptide or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an α₂δ-4 polypeptide or biologically active portion thereof is contacted with a test compound and the ability of the test compound to modulate voltage-gated calcium channel activity is determined. Determining the ability of the test compound to modulate voltage-gated calcium channel activity can be accomplished by monitoring, for example, intracellular calcium, IP3, or diacylglycerol concentration, phosphorylation profile of intracellular proteins, or the activity of a voltage-gated calcium channel-regulated transcription factor. The cell, for example, can be of mammalian origin, e.g., a neuronal cell, a muscle cell or a neuroendocrine cell.

The ability of the test compound to modulate α₂δ-4 binding to a substrate or to bind to α₂δ-4 can also be determined. Determining the ability of the test compound to modulate α₂δ-4 binding to a substrate can be accomplished, for example, by coupling the α₂δ-4 substrate with a radioisotope or enzymatic label such that binding of the α₂δ-4 substrate to α₂δ-4 can be determined by detecting the labeled α₂δ-4 substrate in a complex. Alternatively, α₂δ-4 could be coupled with a radioisotope or enzymatic label to monitor the ability of a test compound to modulate α₂δ-4 binding to an α₂δ-4 substrate in a complex. Determining the ability of the test compound to bind α₂δ-4 can be accomplished, for example, by coupling the compound with a radioisotope or enzymatic label such that binding of the compound to α₂δ-4 can be determined by detecting the labeled α₂δ-4 compound in a complex. For example, compounds (e.g., α₂δ-4 substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., an α₂δ-4 substrate) to interact with α₂δ-4 without the labeling of any of the interactants. For example, a microphysiometer can be used to detect the interaction of a compound with α₂δ-4 without the labeling of either the compound or the α₂δ-4. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and α₂δ-4.

In another embodiment, an assay is a cell-based assay comprising contacting a cell expressing an α₂δ-4 target molecule (e.g., an α₂δ-4 substrate) with a test compound and determining the ability of the test compound to modulate (e.g., stimulate or inhibit) the activity of the α₂δ-4 target molecule. Determining the ability of the test compound to modulate the activity of an α₂δ-4 target molecule can be accomplished, for example, by determining the ability of the α₂δ-4 polypeptide to bind to or interact with the α₂δ-4 target molecule.

Determining the ability of the α₂δ-4 polypeptide, or a biologically active fragment thereof, to bind to or interact with an α₂δ-4 target molecule can be accomplished by one of the methods described above for determining direct binding. In a preferred embodiment, determining the ability of the α₂δ-4 polypeptide to bind to or interact with an α₂δ-4 target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target using an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which an α₂δ-4 polypeptide or biologically active portion thereof is contacted with a test compound and the ability of the test compound to bind to the α₂δ-4 polypeptide or biologically active portion thereof is determined. Preferred biologically active portions of the α₂δ-4 polypeptides to be used in assays of the present invention include fragments which participate in interactions with non-α₂δ-4 molecules, e.g., fragments with high surface probability scores (see, for example, FIG. 42). Binding of the test compound to the α₂δ-4 polypeptide can be determined either directly or indirectly as described above. In a preferred embodiment, the assay includes contacting the α₂δ-4 polypeptide or biologically active portion thereof with a known compound which binds α₂δ-4 to form an assay mixture, contacting the assay mixture with a test compound, and determining the ability of the test compound to interact with an α₂δ-4 polypeptide, wherein determining the ability of the test compound to interact with an α₂δ-4 polypeptide comprises determining the ability of the test compound to preferentially bind to α₂δ-4 or biologically active portion thereof as compared to the known compound.

In another embodiment, the assay is a cell-free assay in which an α₂δ-4 polypeptide or biologically active portion thereof is contacted with a test compound and the ability of the test compound to modulate (e.g., stimulate or inhibit) the activity of the α₂δ-4 polypeptide or biologically active portion thereof is determined. Determining the ability of the test compound to modulate the activity of an α₂δ-4 polypeptide can be accomplished, for example, by determining the ability of the α₂δ-4 polypeptide to bind to an α₂δ-4 target molecule by one of the methods described above for determining direct binding. Determining the ability of the α₂δ-4 polypeptide to bind to an α₂δ-4 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an α₂δ-4 polypeptide can be accomplished by determining the ability of the α₂δ-4 polypeptide to further modulate the activity of a downstream effector of an α₂δ-4 target molecule. For example, the activity of the effector molecule on an appropriate target can be determined or the binding of the effector to an appropriate target can be determined as previously described.

In yet another embodiment, the cell-free assay involves contacting an α₂δ-4 polypeptide or biologically active portion thereof with a known compound which binds the α₂δ-4 polypeptide to form an assay mixture, contacting the assay mixture with a test compound, and determining the ability of the test compound to interact with the α₂δ-4 polypeptide, wherein determining the ability of the test compound to interact with the α₂δ-4 polypeptide comprises determining the ability of the α₂δ-4 polypeptide to preferentially bind to or modulate the activity of an α₂δ-4 target molecule.

In more than one embodiment of the above assay methods of the present invention, it may be desirable to immobilize either α₂δ-4 or its target molecule to facilitate separation of complexed from uncomplexed forms of one or both of the proteins, as well as to accommodate automation of the assay. Binding of a test compound to an α₂δ-4 polypeptide, or interaction of an α₂δ-4 polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/α₂δ-4 fusion proteins or glutathione-S-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or α₂δ-4 polypeptide, and the mixture incubated under conditions conducive to complex formation (e.g., at physiological conditions for salt and pH). Following incubation, the beads or micrometer plate wells are washed to remove any unbound components, the matrix immobilized in the case of beads, complex determined either directly or indirectly, for example, as described above. Alternatively, the complexes can be dissociated from the matrix, and the level of α₂δ-4 binding or activity determined using standard techniques.

Other techniques for immobilizing proteins on matrices can also be used in the screening assays of the invention. For example, either an α₂δ-4 polypeptide or an α₂δ-4 target molecule can be immobilized utilizing conjugation of biotin and streptavidin. Biotinylated α₂δ-4 polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with α₂δ-4 polypeptide or target molecules but which do not interfere with binding of the α₂δ-4 polypeptide to its target molecule can be derivatized to the wells of the plate, and unbound target or α₂δ-4 polypeptide trapped in the wells by antibody conjugation. Methods for detecting such complexes, in addition to those described above for the GST-immobilized complexes, include immunodetection of complexes using antibodies reactive with the α₂δ-4 polypeptide or target molecule, as well as enzyme-linked assays which rely on detecting an enzymatic activity associated with the α₂δ-4 polypeptide or target molecule.

In another embodiment, modulators of α₂δ-4 expression are identified in a method wherein a cell is contacted with a candidate compound and the expression of α₂δ-4 mRNA or polypeptide in the cell is determined. The level of expression of α₂δ-4 mRNA or polypeptide in the presence of the candidate compound is compared to the level of expression of α₂δ-4 mRNA or polypeptide in the absence of the candidate compound. The candidate compound can then be identified as a modulator of α₂δ-4 expression based on this comparison. For example, when expression of α₂δ-4 mRNA or polypeptide is greater (statistically significantly greater) in the presence of the candidate compound than in its absence, the candidate compound is identified as a stimulator of α₂δ-4 mRNA or polypeptide expression. Alternatively, when expression of α₂δ-4 mRNA or polypeptide is less (statistically significantly less) in the presence of the candidate compound than in its absence, the candidate compound is identified as an inhibitor of α₂δ-4 mRNA or polypeptide expression. The level of α₂δ-4 mRNA or polypeptide expression in the cells can be determined by methods described herein for detecting α₂δ-4 mRNA or polypeptide.

In yet another aspect of the invention, the α₂δ-4 polypeptides can be used as “bait proteins” in a two-hybrid assay or three-hybrid assay (see, e.g., U.S. Pat. No. 5,283,317; Zervos et al. (1993) i Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with α₂δ-4 (“α₂δ-4-binding proteins” or “α₂δ-4-bp”) and are involved in α₂δ-4 activity. Such α₂δ-4-binding proteins are also likely to be involved in the propagation of signals by the α₂δ-4 polypeptides or α₂δ-4 targets as, for example, downstream elements of an α₂δ-4-mediated signaling pathway. Alternatively, such α₂δ-4-binding proteins are likely to be α₂δ-4 inhibitors.

The two-hybrid system is based on the modular nature of most transcription factors, which consist of separable DNA-binding and activation domains. Briefly, the assay utilizes two different DNA constructs. In one construct, the gene that codes for an α₂δ-4 polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an α₂δ-4-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the α₂δ-4 polypeptide.

In another aspect, the invention pertains to a combination of two or more of the assays described herein. For example, a modulating agent can be identified using a cell-based or a cell free assay, and the ability of the agent to modulate the activity of an α₂δ-4 polypeptide can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., an α₂δ-4 modulating agent, an antisense α₂δ-4 nucleic acid molecule, an α₂δ-4-specific antibody, or an α₂δ-4-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the α₂δ-4 nucleotide sequences, described herein, can be used to map the location of the α₂δ-4 genes on a chromosome. The mapping of the α₂δ-4 sequences to chromosomes is an important first step in correlating these sequences with genes associated with disease.

Briefly, α₂δ-4 genes can be mapped to chromosomes by preparing PCR primers (preferably 15-25 bp in length) from the α₂δ-4 nucleotide sequences. Computer analysis of the α₂δ-4 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the α₂δ-4 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the α₂δ-4 nucleotide sequences to design oligonucleotide primers, sublocalization can be achieved with panels of fragments from specific chromosomes. Other mapping strategies which can similarly be used to map an α₂δ-4 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the α₂δ-4 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The α₂δ-4 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the α₂δ-4 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The α₂δ-4 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 can comfortably provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from α₂δ-4 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of α₂δ-4 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the α₂δ-4 nucleotide sequences or portions thereof, e.g., fragments derived from the noncoding regions of SEQ ID NO:1 having a length of at least 20 bases, preferably at least 30 bases.

The α₂δ-4 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such α₂δ-4 probes can be used to identify tissue by species and/or by organ type.

In a similar fashion, these reagents, e.g., α₂δ-4 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining α₂δ-4 polypeptide and/or nucleic acid expression as well as α₂δ-4 activity, in the context of a biological sample (e.g., blood, serum, cells, tissue) to thereby determine whether an individual is afflicted with a disease or disorder, or is at risk of developing a disorder, associated with aberrant or unwanted α₂δ-4 expression or activity. The invention also provides for prognostic (or predictive) assays for determining whether an individual is at risk of developing a disorder associated with α₂δ-4 polypeptide, nucleic acid expression or activity. For example, mutations in an α₂δ-4 gene can be assayed in a biological sample. Such assays can be used for prognostic or predictive purpose to thereby prophylactically treat an individual prior to the onset of a disorder characterized by or associated with α₂δ-4 polypeptide, nucleic acid expression or activity.

Another aspect of the invention pertains to monitoring the influence of agents (e.g., drugs, compounds) on the expression or activity of α₂δ-4 in clinical trials.

These and other agents are described in further detail in the following sections.

1. Diagnostic Assays

An exemplary method for detecting the presence or absence of α₂δ-4 polypeptide or nucleic acid in a biological sample involves obtaining a biological sample from a test subject and contacting the biological sample with a compound or an agent capable of detecting α₂δ-4 polypeptide or nucleic acid (e.g., mRNA, or genomic DNA) that encodes α₂δ-4 polypeptide such that the presence of α₂δ-4 polypeptide or nucleic acid is detected in the biological sample. In another aspect, the present invention provides a method for detecting the presence of α₂δ-4 activity in a biological sample by contacting the biological sample with an agent capable of detecting an indicator of α₂δ-4 activity such that the presence of α₂δ-4 activity is detected in the biological sample. A preferred agent for detecting α₂δ-4 mRNA or genomic DNA is a labeled nucleic acid probe capable of hybridizing to α₂δ-4 mRNA or genomic DNA. The nucleic acid probe can be, for example, the α₂δ-4 nucleic acid set forth in SEQ ID NO:43 or 45, or the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to α₂δ-4 mRNA or genomic DNA. Other suitable probes for use in the diagnostic assays of the invention are described herein.

A preferred agent for detecting α₂δ-4 polypeptide is an antibody capable of binding to α₂δ-4 polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect α₂δ-4 mRNA, polypeptide, or genomic DNA in a biological sample in vitro as well as in vivo. For example, in vitro techniques for detection of α₂δ-4 mRNA include Northern hybridizations and in situ hybridizations. In vitro techniques for detection of α₂δ-4 polypeptide include enzyme linked immunosorbent assays (ELISAs), Western blots, immunoprecipitations and immunofluorescence. In vitro techniques for detection of α₂δ-4 genomic DNA include Southern hybridizations. Furthermore, in vivo techniques for detection of α₂δ-4 polypeptide include introducing into a subject a labeled anti-α₂δ-4 antibody. For example, the antibody can be labeled with a radioactive marker whose presence and location in a subject can be detected by standard imaging techniques.

The present invention also provides diagnostic assays for identifying the presence or absence of a genetic alteration characterized by at least one of (i) aberrant modification or mutation of a gene encoding an α₂δ-4 polypeptide; (ii) aberrant expression of a gene encoding an α₂δ-4 polypeptide; (iii) mis-regulation of the gene; and (iii) aberrant post-translational modification of an α₂δ-4 polypeptide, wherein a wild-type form of the gene encodes a polypeptide with an α₂δ-4 activity. “Misexpression or aberrant expression”, as used herein, refers to a non-wild type pattern of gene expression, at the RNA or protein level. It includes, but is not limited to, expression at non-wild type levels (e.g., over or under expression); a pattern of expression that differs from wild type in terms of the time or stage at which the gene is expressed (e.g., increased or decreased expression (as compared with wild type) at a predetermined developmental period or stage); a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene (e.g., a pattern of increased or decreased expression (as compared with wild type) in the presence of an increase or decrease in the strength of the stimulus).

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting α₂δ-4 polypeptide, mRNA, or genomic DNA, such that the presence of α₂δ-4 polypeptide, mRNA or genomic DNA is detected in the biological sample, and comparing the presence of α₂δ-4 polypeptide, mRNA or genomic DNA in the control sample with the presence of α₂δ-4 polypeptide, mRNA or genomic DNA in the test sample.

The invention also encompasses kits for detecting the presence of α₂δ-4 in a biological sample. For example, the kit can comprise a labeled compound or agent capable of detecting α₂δ-4 polypeptide or mRNA in a biological sample; means for determining the amount of α₂δ-4 in the sample; and means for comparing the amount of α₂δ-4 in the sample with a standard. The compound or agent can be packaged in a suitable container. The kit can further comprise instructions for using the kit to detect α₂δ-4 polypeptide or nucleic acid.

2. Prognostic Assays

The diagnostic methods described herein can furthermore be utilized to identify subjects having or at risk of developing a disease or disorder associated with aberrant or unwanted α₂δ-4 expression or activity. As used herein, the term “aberrant” includes an α₂δ-4 expression or activity which deviates from the wild type α₂δ-4 expression or activity. Aberrant expression or activity includes increased or decreased expression or activity, as well as expression or activity which does not follow the wild type developmental pattern of expression or the subcellular pattern of expression. For example, aberrant α₂δ-4 expression or activity is intended to include the cases in which a mutation in the α₂δ-4 gene causes the α₂δ-4 gene to be under-expressed or over-expressed and situations in which such mutations result in a non-functional α₂δ-4 polypeptide or a polypeptide which does not function in a wild-type fashion, e.g, a polypeptide which does not interact with an α₂δ-4 substrate, e.g., a voltage-gated calcium channel subunit or ligand, or one which interacts with a non-α₂δ-4 substrate, e.g. a non-voltage-gated calcium channel subunit or ligand. As used herein, the term “unwanted” includes an unwanted phenomenon involved in a biological response, such as cellular proliferation. For example, the term unwanted includes an α₂δ-4 expression or activity which is undesirable in a subject.

The assays described herein, such as the preceding diagnostic assays or the following assays, can be utilized to identify a subject having or at risk of developing a disorder associated with a misregulation in α₂δ-4 polypeptide activity or nucleic acid expression, such as a muscle disorder or a CNS disorder (e.g., a neurodegenerative disorder, a pain disorder, or a cellular proliferation, growth, differentiation, or migration disorder). Alternatively, the prognostic assays can be utilized to identify a subject having or at risk for developing a disorder associated with a misregulation in α₂δ-4 polypeptide activity or nucleic acid expression, such as a CNS disorder, a pain disorder, or a cellular proliferation, growth, differentiation, or migration disorder. Thus, the present invention provides a method for identifying a disease or disorder associated with aberrant or unwanted α₂δ-4 expression or activity in which a test sample is obtained from a subject and α₂δ-4 polypeptide or nucleic acid (e.g., mRNA or genomic DNA) is detected, wherein the presence of α₂δ-4 polypeptide or nucleic acid is diagnostic for a subject having or at risk of developing a disease or disorder associated with aberrant or unwanted α₂δ-4 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted α₂δ-4 expression or activity. For example, such methods can be used to determine whether a subject can be effectively treated with an agent for a CNS disorder, pain disorder, or a cellular proliferation, growth, differentiation, or migration disorder. Thus, the present invention provides methods for determining whether a subject can be effectively treated with an agent for a disorder associated with aberrant or unwanted α₂δ-4 expression or activity in which a test sample is obtained and α₂δ-4 polypeptide or nucleic acid expression or activity is detected (e.g., wherein the abundance of α₂δ-4 polypeptide or nucleic acid expression or activity is diagnostic for a subject that can be administered the agent to treat a disorder associated with aberrant or unwanted α₂δ-4 expression or activity).

The methods of the invention can also be used to detect genetic alterations in an α₂δ-4 gene, thereby determining if a subject with the altered gene is at risk for a disorder characterized by misregulation in α₂δ-4 polypeptide activity or nucleic acid expression, such as a CNS disorder, pain disorder, or a disorder of cellular growth, differentiation, or migration. In preferred embodiments, the methods include detecting, in a sample of cells from the subject, the presence or absence of a genetic alteration characterized by at least one of an alteration affecting the integrity of a gene encoding an α₂δ-4-polypeptide, or the mis-expression of the α₂δ-4 gene. For example, such genetic alterations can be detected by ascertaining the existence of at least one of 1) a deletion of one or more nucleotides from an α₂δ-4 gene; 2) an addition of one or more nucleotides to an α₂δ-4 gene; 3) a substitution of one or more nucleotides of an α₂δ-4 gene, 4) a chromosomal rearrangement of an α₂δ-4 gene; 5) an alteration in the level of a messenger RNA transcript of an α₂δ-4 gene, 6) aberrant modification of an α₂δ-4 gene, such as of the methylation pattern of the genomic DNA, 7) the presence of a non-wild type splicing pattern of a messenger RNA transcript of an α₂δ-4 gene, 8) a non-wild type level of an α₂δ-4-polypeptide, 9) allelic loss of an α₂δ-4 gene, and 10) inappropriate post-translational modification of an α₂δ-4-polypeptide. As described herein, there are a large number of assays known in the art which can be used for detecting alterations in an α₂δ-4 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the α₂δ-4-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to an α₂δ-4 gene under conditions such that hybridization and amplification of the α₂δ-4-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an α₂δ-4 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in α₂δ-4 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in α₂δ-4 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the α₂δ-4 gene and detect mutations by comparing the sequence of the sample α₂δ-4 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the α₂δ-4 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type α₂δ-4 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in α₂δ-4 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an α₂δ-4 sequence, e.g., a wild-type α₂δ-4 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in α₂δ-4 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal Tech. Appl. 9:73-79). Single-stranded DNA fragments of sample and control α₂δ-4 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an α₂δ-4 gene.

Furthermore, any cell type or tissue in which α₂δ-4 is expressed may be utilized in the prognostic assays described herein.

3. Monitoring of Effects During Clinical Trials

Monitoring the influence of agents (e.g., drugs) on the expression or activity of an α₂δ-4 polypeptide (e.g., the modulation of membrane excitability) can be applied not only in basic drug screening, but also in clinical trials. For example, the effectiveness of an agent determined by a screening assay as described herein to increase α₂δ-4 gene expression, polypeptide levels, or upregulate α₂δ-4 activity, can be monitored in clinical trials of subjects exhibiting decreased α₂δ-4 gene expression, polypeptide levels, or downregulated α₂δ-4 activity. Alternatively, the effectiveness of an agent determined by a screening assay to decrease α₂δ-4 gene expression, polypeptide levels, or downregulate α₂δ-4 activity, can be monitored in clinical trials of subjects exhibiting increased α₂δ-4 gene expression, polypeptide levels, or upregulated α₂δ-4 activity. In such clinical trials, the expression or activity of an α₂δ-4 gene, and preferably, other genes that have been implicated in, for example, an α₂δ-4-associated disorder can be used as a “read out” or markers of the phenotype of a particular cell.

For example, and not by way of limitation, genes, including α₂δ-4, that are modulated in cells by treatment with an agent (e.g., compound, drug or small molecule) which modulates α₂δ-4 activity (e.g., identified in a screening assay as described herein) can be identified. Thus, to study the effect of agents on α₂δ-4-associated disorders (e.g, disorders characterized by deregulated signaling or membrane excitation), for example, in a clinical trial, cells can be isolated and RNA prepared and analyzed for the levels of expression of α₂δ-4 and other genes implicated in the α₂δ-4-associated disorder, respectively. The levels of gene expression (e.g., a gene expression pattern) can be quantified by northern blot analysis or RT-PCR, as described herein, or alternatively by measuring the amount of polypeptide produced, by one of the methods as described herein, or by measuring the levels of activity of α₂δ-4 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an α₂δ-4 polypeptide, mRNA, or genomic DNA in the preadministration sample; (iii) obtaining one or more post-administration samples from the subject; (iv) detecting the level of expression or activity of the α₂δ-4 polypeptide, mRNA, or genomic DNA in the post-administration samples; (v) comparing the level of expression or activity of the α₂δ-4 polypeptide, mRNA, or genomic DNA in the pre-administration sample with the α₂δ-4 polypeptide, mRNA, or genomic DNA in the post administration sample or samples; and (vi) altering the administration of the agent to the subject accordingly. For example, increased administration of the agent may be desirable to increase the expression or activity of α₂δ-4 to higher levels than detected, i.e., to increase the effectiveness of the agent. Alternatively, decreased administration of the agent may be desirable to decrease expression or activity of α₂δ-4 to lower levels than detected, i.e. to decrease the effectiveness of the agent. According to such an embodiment, α₂δ-4 expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted α₂δ-4 expression or activity, e.g. a muscle disorder, a CNS disorder, a pain disorder, or a cellular proliferation, growth, differentiation, or migration disorder. The term “treatment” as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the α₂δ-4 molecules of the present invention or α₂δ-4 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted α₂δ-4 expression or activity, by administering to the subject an α₂δ-4 or an agent which modulates α₂δ-4 expression or at least one α₂δ-4 activity. Subjects at risk for a disease which is caused or contributed to by aberrant or unwanted α₂δ-4 expression or activity can be identified by, for example, any or a combination of diagnostic or prognostic assays as described herein. Administration of a prophylactic agent can occur prior to the manifestation of symptoms characteristic of the α₂δ-4 aberrancy, such that a disease or disorder is prevented or, alternatively, delayed in its progression. Depending on the type of α₂δ-4 aberrancy, for example, an α₂δ-4, α₂δ-4 agonist or α₂δ-4 antagonist agent can be used for treating the subject. The appropriate agent can be determined based on screening assays described herein.

2. Therapeutic Methods

Another aspect of the invention pertains to methods of modulating α₂δ-4 expression or activity for therapeutic purposes. Accordingly, in an exemplary embodiment, the modulatory method of the invention involves contacting a cell capable of expressing α₂δ-4 with an agent that modulates one or more of the activities of α₂δ-4 polypeptide activity associated with the cell, such that α₂δ-4 activity in the cell is modulated. An agent that modulates α₂δ-4 polypeptide activity can be an agent as described herein, such as a nucleic acid or a polypeptide, a naturally-occurring target molecule of an α₂δ-4 polypeptide (e.g., an α₂δ-4 substrate), an α₂δ-4 antibody, an α₂δ-4 agonist or antagonist, a peptidomimetic of an α₂δ-4 agonist or antagonist, or other small molecule. In one embodiment, the agent stimulates one or more α₂δ-4 activities. Examples of such stimulatory agents include active α₂δ-4 polypeptide and a nucleic acid molecule encoding α₂δ-4 that has been introduced into the cell. In another embodiment, the agent inhibits one or more α₂δ-4 activities. Examples of such inhibitory agents include antisense α₂δ-4 nucleic acid molecules, anti-α₂δ-4 antibodies, and α₂δ-4 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of an α₂δ-4 polypeptide or nucleic acid molecule. In one embodiment, the method involves administering an agent (e.g., an agent identified by a screening assay described herein), or combination of agents that modulates (e.g., upregulates or downregulates) α₂δ-4 expression or activity. In another embodiment, the method involves administering an α₂δ-4 polypeptide or nucleic acid molecule as therapy to compensate for reduced, aberrant, or unwanted α₂δ-4 expression or activity.

Stimulation of α₂δ-4 activity is desirable in situations in which α₂δ-4 is abnormally downregulated and/or in which increased α₂δ-4 activity is likely to have a beneficial effect. Likewise, inhibition of α₂δ-4 activity is desirable in situations in which α₂δ-4 is abnormally upregulated and/or in which decreased α₂δ-4 activity is likely to have a beneficial effect.

3. Pharmacogenomics

The α₂δ-4 molecules of the present invention, as well as agents, or modulators which have a stimulatory or inhibitory effect on α₂δ-4 activity (e.g., α₂δ-4 gene expression) as identified by a screening assay described herein can be administered to individuals to treat (prophylactically or therapeutically) α₂δ-4-associated disorders (e.g., proliferative disorders) associated with aberrant or unwanted α₂δ-4 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an α₂δ-4 molecule or α₂δ-4 modulator as well as tailoring the dosage and/or therapeutic regimen of treatment with an α₂δ-4 molecule or α₂δ-4 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., an α₂δ-4 polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an α₂δ-4 molecule or α₂δ-4 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an α₂δ-4 molecule or α₂δ-4 modulator, such as a modulator identified by one of the exemplary screening assays described herein.

4. Use of α₂δ-4 Molecules as Surrogate Markers

The α₂δ-4 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the α₂δ-4 molecules of the invention may be detected, and may be correlated with one or more biological states in vivo. For example, the α₂δ-4 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The α₂δ-4 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., an α₂δ-4 marker) transcription or expression, the amplified marker may be in a quantity which is more readily detectable than the drug itself. Also, the marker may be more easily detected due to the nature of the marker itself, for example, using the methods described herein, anti-α₂δ-4 antibodies may be employed in an immune-based detection system for an α₂δ-4 polypeptide marker, or α₂δ-4-specific radiolabeled probes may be used to detect an α₂δ-4 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20. The α₂δ-4 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or polypeptide (e.g., α₂δ-4 polypeptide or RNA) for specific tumor markers in a subject, a drug or course of treatment may be selected that is optimized for the treatment of the specific tumor likely to be present in the subject. Similarly, the presence or absence of a specific sequence mutation in α₂δ-4 DNA may correlate α₂δ-4 drug response. The use of pharmacogenomic markers therefore permits the application of the most appropriate treatment for each subject without having to administer the therapy.

E. Electronic Apparatus Readable Media and Arrays

Electronic apparatus readable media comprising α₂δ-4 sequence information is also provided. As used herein, “α₂δ-4 sequence information” refers to any nucleotide and/or amino acid sequence information particular to the α₂δ-4 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said α₂δ-4 sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon α₂δ-4 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the α₂δ-4 sequence information.

A variety of software programs and formats can be used to store the sequence information on the electronic apparatus readable medium. For example, the sequence information can be represented in a word processing text file, formatted in commercially-available software such as WordPerfect and Microsoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon the α₂δ-4 sequence information.

By providing α₂δ-4 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a α₂δ-4-associated disease or disorder or a pre-disposition to a α₂δ-4-associated disease or disorder, wherein the method comprises the steps of determining α₂δ-4 sequence information associated with the subject and based on the α₂δ-4 sequence information, determining whether the subject has a α₂δ-4-associated disease or disorder or a pre-disposition to a α₂δ-4-associated disease or disorder and/or recommending a particular treatment for the disease, disorder or pre-disease condition.

The present invention further provides in an electronic system and/or in a network, a method for determining whether a subject has a α₂δ-4-associated disease or disorder or a pre-disposition to a disease associated with a α₂δ-4 wherein the method comprises the steps of determining α₂δ-4 sequence information associated with the subject, and based on the -4 sequence information, determining whether the subject has a α₂δ-4-associated disease or disorder or a pre-disposition to a α₂δ-4-associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a α₂δ-4-associated disease or disorder or a pre-disposition to a α₂δ-4 associated disease or disorder associated with α₂δ-4, said method comprising the steps of receiving α₂δ-4 sequence information from the subject and/or information related thereto, receiving phenotypic information associated with the subject, acquiring information from the network corresponding to α₂δ-4 and/or a α₂δ-4-associated disease or disorder, and based on one or more of the phenotypic information, the α₂δ-4 information (e.g., sequence information and/or information related thereto), and the acquired information, determining whether the subject has a α₂δ-4-associated disease or disorder or a pre-disposition to a α₂δ-4-associated disease or disorder (e.g., a cellular growth or proliferation disease or disorder, for example, cancer). The method may further comprise the step of recommending a particular treatment for the disease, disorder or pre-disease condition.

The present invention also provides a business method for determining whether a subject has a α₂δ-4-associated disease or disorder or a pre-disposition to a α₂δ-4-associated disease or disorder, said method comprising the steps of receiving information related to α₂δ-4 (e.g., sequence information and/or information related thereto), receiving phenotypic information associated with the subject, acquiring information from the network related to α₂δ-4 and/or related to a α₂δ-4-associated disease or disorder, and based on one or more of the phenotypic information, the α₂δ-4 information, and the acquired information, determining whether the subject has a α₂δ-4-associated disease or disorder or a pre-disposition to a α₂δ-4-associated disease or disorder. The method may further comprise the step of recommending a particular treatment for the disease, disorder or pre-disease condition.

The invention also includes an array comprising a α₂δ-4 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be α₂δ-4. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a α₂δ-4-associated disease or disorder, progression of α₂δ-4-associated disease or disorder, and processes, such a cellular transformation associated with the α₂δ-4-associated disease or disorder.

The array is also useful for ascertaining the effect of the expression of a gene on the expression of other genes in the same cell or in different cells (e.g., ascertaining the effect of α₂δ-4 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including α₂δ-4) that could serve as a molecular target for diagnosis or therapeutic intervention.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human α₂δ-4 cDNA

In this example, the identification and characterization of the gene encoding human α₂δ-4 (clone 25658) is described.

Isolation of the Human α₂δ-4 cDNA

The invention is based, at least in part, on the discovery of a human gene encoding a novel polypeptide, referred to herein as human α₂δ-4. The entire sequence of the human clone 25658 was determined and found to contain an open reading frame termed human “α₂δ-4.” The nucleotide sequence of the human α₂δ-4 gene is set forth in FIGS. 41A-D and in the Sequence Listing as SEQ ID NO:1. The amino acid sequence of the human α₂δ-4 expression product is set forth in FIGS. 41A-D and in the Sequence Listing as SEQ ID NO: 2. The α₂δ-4 polypeptide comprises about 1223 amino acids. The coding region (open reading frame) of SEQ ID NO:1 is set forth as SEQ ID NO:3. Clone 25658, comprising the coding region of human α₂δ-4, was deposited with the American Type Culture Collection (ATCC®), 10801 University Boulevard, Manassas, Va. 20110-2209, on ______, and assigned Accession No. ______.

Analysis of the Human α₂δ-4 Molecules

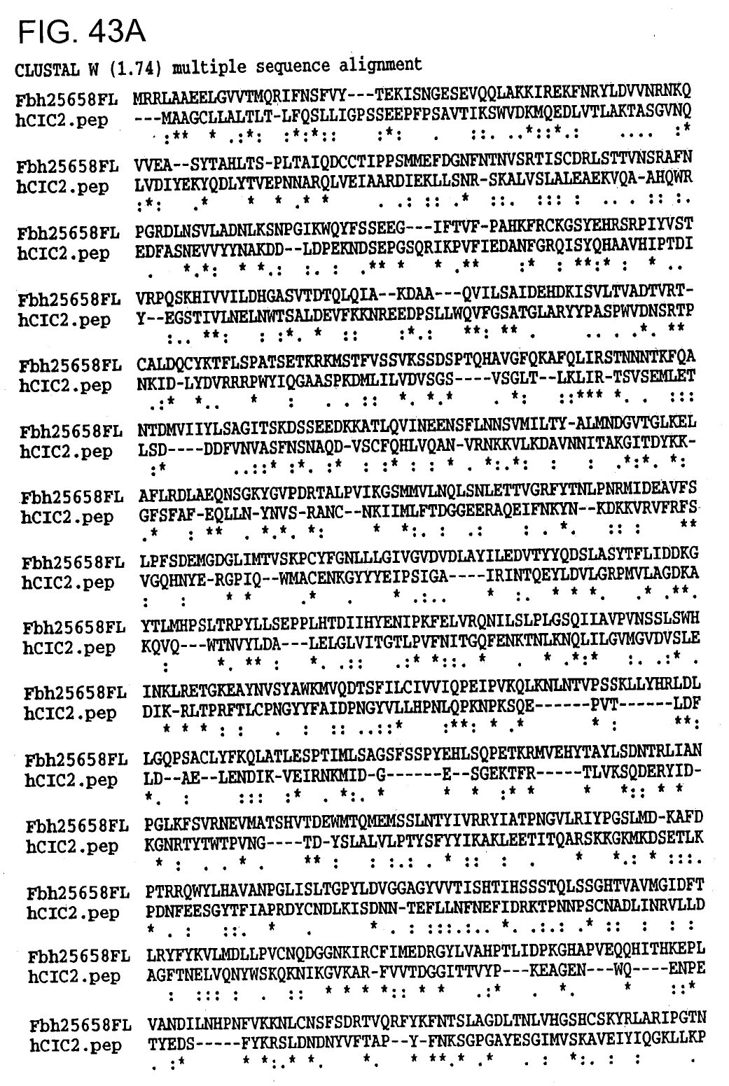

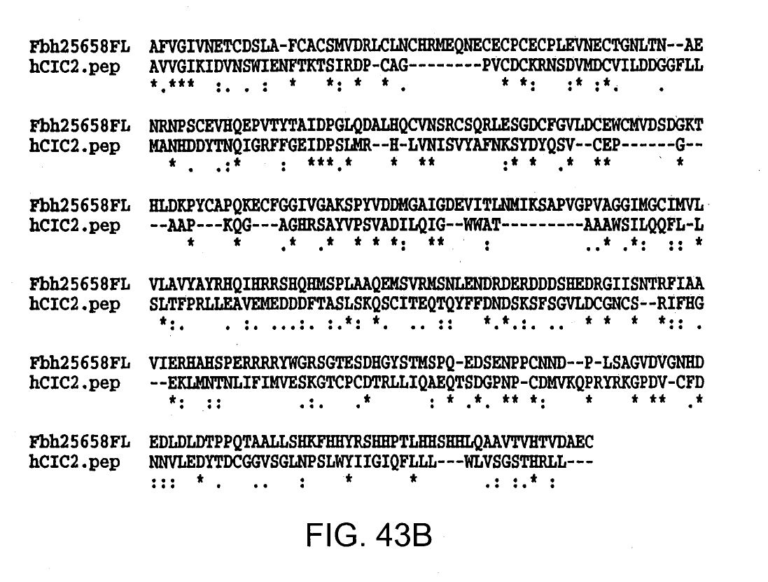

The human α₂δ-4 amino acid sequence (SEQ ID NO:44) was aligned with the amino acid sequence of the human dihydropyridine-sensitive L-type calcium channel α₂δ subunit protein CIC2 (SwissProt Accession No. P54289), using the CLUSTAL W (1.74) alignment program. The results of the alignment are set forth in FIGS. 43A-43B.

A search using the polypeptide sequence of SEQ ID NO:44 was also performed against a proprietary HMM database resulting in the identification of a potential von Willebrand factor type A domain in the amino acid sequence of α₂δ-4 at about residues 175-371 of SEQ ID NO:44 (score=6.1).

A MEMSAT analysis of the polypeptide sequence of SEQ ID NO:44 was also performed, predicting two potential transmembrane domains in the amino acid sequence of α₂δ-4 (SEQ ID NO:44) at about residues 422-442, and 1048-1066.

Searches of the amino acid sequence of α₂δ-4 were further performed against the Prosite database. These searches resulted in the identification in the amino acid sequence of α₂δ-4 of a number of potential N-glycosylation sites(e.g., 94-97, 278-281, 322-325, 517-520, 536-539, 854-857, 889-892, 934-937), a number of potential cAMP- and cGMP-dependent protein kinase phosphorylation sites (e.g., 244-247, 307-310), a number of potential protein kinase C phosphorylation sites (e.g., 24-26, 170-172, 221-223, 242-244, 252-254, 298-300, 354-356, 530-532, 573-575, 623-625, 649-651, 704-706, 762-764, 817-819, 843-845, 974-976, 1087-1089, 1191-1193), a number of potential casein kinase II phosphorylation sites (e.g., 379-382, 530-533, 613-616, 619-622, 666-669, 695-698, 801-804, 817-820, 841-844, 901-904, 936-939, 955-958, 999-1002, 1091-1094, 1138-1141, 1150-1153), a number of potential tyrosine kinase phosphorylation sites (e.g., 56-63, 998-1005), a number of potential N-myristoylation sites (e.g., 89-94, 185-190, 413-418, 585-590, 645-650, 758-763, 867-872, 933-938, 960-965, 984-989, 1014-1019, 1050-1055, 1109-1114, 1168-1173), a potential prokaryotic membrane lipoprotein lipid attachment site(e.g., 1045-1055), and a potential cytochrome c family heme-binding site signature (e.g., 907-912).

To further identify potential structural and/or functional properties in a protein of interest, the amino acid sequence of the protein is searched against a database of annotated protein domains (e.g., the ProDom database) using the default parameters (available at http://www.toulouse.inra.fr/prodom.html). A search of the amino acid sequence of human SEQ ID NO:44 was performed against the ProDom database. This search resulted in the local alignment of the human SEQ ID NO:44 protein with various calcium channel proteins.

Tissue Distribution of Human α₂δ-4 mRNA

This example describes the tissue distribution of human α₂δ-4 mRNA, as may be determined by Polymerase Chain Reaction (PCR) on cDNA libraries using oligonucleotide primers based on the human α₂δ-4 sequence.

For in situ analysis, various tissues, e.g. tissues obtained from muscles or brain, are first frozen on dry ice. Ten-micrometer-thick sections of the tissues are postfixed with 4% formaldehyde in DEPC treated 1×phosphate-buffered saline at room temperature for 10 minutes before being rinsed twice in DEPC 1×phosphate-buffered saline and once in 0.1 M triethanolamine-HCl (pH 8.0). Following incubation in 0.25% acetic anhydride-0.1 M triethanolamine-HCl for 10 minutes, sections are rinsed in DEPC 2×SSC (1×SSC is 0.15M NaCl plus 0.015M sodium citrate). Tissue is then dehydrated through a series of ethanol washes, incubated in 100% chloroform for 5 minutes, and then rinsed in 100% ethanol for 1 minute and 95% ethanol for 1 minute and allowed to air dry.

Hybridizations are performed with ³⁵S-radiolabeled (5×10⁷ cpm/ml) cRNA probes. Probes are incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides are washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides are then rinsed with 2×SSC at room temperature, washed with 2×SSC at 50° C. for 1 hour, washed with 0.2×SSC at 55° C. for 1 hour, and 0.2×SSC at 60° C. for 1 hour. Sections are then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

### Example 2

### Expression of Recombinant α₂δ-4 Polypeptide in Bacterial Cells

In this example, human α₂δ-4 is expressed as a recombinant glutathione-S-transferase (GST) fusion polypeptide in E. coli and the fusion polypeptide is isolated and characterized. Specifically, α₂δ-4 is fused to GST and this fusion polypeptide is expressed in E. coli, e.g., strain PEB199. Expression of the GST-α₂δ-4 fusion polypeptide in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant α₂δ-4 Polypeptide in COS Cells

To express the human α₂δ-4 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire α₂δ-4 polypeptide and an HA tag (Wilson et al. (1984) Cell 37:767) or a FLAG tag fused in-frame to its 3′ end of the fragment is cloned into the polylinker region of the vector, thereby placing the expression of the recombinant polypeptide under the control of the CMV promoter.

To construct the plasmid, the human α₂δ-4 DNA sequence is amplified by PCR using two primers. The 5′ primer contains the restriction site of interest followed by approximately twenty nucleotides of the α₂δ-4 coding sequence starting from the initiation codon; the 3′ end sequence contains complementary sequences to the other restriction site of interest, a translation stop codon, the HA tag or FLAG tag and the last 20 nucleotides of the α₂δ-4 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the α₂δ-4 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the human α₂δ-4-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the IC54420 polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the human α₂δ-4 coding sequence is cloned directly into the polylinker of the pCDNA/Amp vector using the appropriate restriction sites. The resulting plasmid is transfected into COS cells in the manner described above, and the expression of the α₂δ-4 polypeptide is detected by radiolabeling and immunoprecipitation using an α₂δ-4-specific monoclonal antibody.

### X. 8099, 46455, 54414, 53763, 67076, 67102, 44181, 67084FL, AND 67084 ALT, HUMAN PROTEINS AND METHODS OF USE THEREOF

### Background of the Invention

Cellular membranes serve to differentiate the contents of a cell from the surrounding environment, and may also serve as effective barriers against the unregulated influx of hazardous or unwanted compounds, and the unregulated efflux of desirable compounds. Membranes are by nature impervious to the unfacilitated diffusion of hydrophilic compounds such as proteins, water molecules, and ions due to their structure: a bilayer of lipid molecules in which the polar head groups face outward (towards the exterior and interior of the cell) and the nonpolar tails face inward (at the center of bilayer, forming a hydrophobic core). Membranes enable a cell to maintain a relatively higher intracellular concentration of desired compounds and a relatively lower intracellular concentration of undesired compounds than are contained within the surrounding environment.

Membranes also present a structural difficulty for cells, in that most desired compounds cannot readily enter the cell, nor can most waste products readily exit the cell through this lipid bilayer. The import and export of such compounds is regulated by proteins which are embedded (singly or in complexes) in the cellular membrane. Two mechanisms exists whereby membrane proteins allow the passage of compounds: non-mediated and mediated transport. Simple diffusion is an example of non-mediated transport, while facilitated diffusion and active transport are examples of mediated transport. Permeases, porters, translocases, translocators, and transporters are proteins that engage in mediated transport (Voet and Voet (1990) Biochemistry, John Wiley and Sons, Inc., New York, N.Y. pp. 484-505).

Sugar transporters are members of the major facilitator superfamily of transporters. These transporters are passive in the sense that they are driven by the substrate concentration gradient and they exhibit distinct kinetics as well as sugar substrate specificity. Members of this family share several characteristics: (1) they contain twelve transmembrane domains separated by hydrophilic loops; (2) they have intracellular N- and C-termini; and (3) they are thought to function as oscillating pores. The transport mechanism occurs via sugar binding to the exofacial binding site of the transporter, which is thought to trigger a conformational change causing the sugar binding site to re-orient to the endofacial conformation, allowing the release of substrate. These transporters are specific for various sugars and are found in both prokaryotes and eukaryotes. In mammals, sugar transporters transport various monosaccharides across the cell membrane (Walmsley et al. (1998) Trends in Biochem. Sci. 23:476-481; Barrett et al. (1999) Curr. Op. Cell Biol. 11:496-502).

At least nine mammalian glucose transporters have been identified, GLUT1-GLUT9, which are expressed in a tissue-specific manner (e.g., in brain, erythrocyte, kidney, muscle, and adipose tissues) (Shepherd et al. (1999) N. Engl. J. Med. 341:248-257; Doege et al. (2000) Biochem. J. 350:771-776). Some GLUT proteins have been shown to be present in low amounts at the plasma membrane during the basal state, at which time large amounts are sequestered in intracellular vesicle stores. Stimulatory molecules specific for each GLUT (such as insulin) regulate the translocation of the GLUT-containing vesicles to the plasma membrane. The vesicles fuse at the membrane and subsequently expose the GLUT protein to the extracellular milieu to allow glucose (and other monosaccharide) transport into the cell (Walmsley et al. (1998) Trends in Biochem. Sci. 23:476-481; Barrett et al. (1999) Curr. Op. Cell Biol. 11:496-502). Other GLUT transporters play a role in constitutive sugar transport.

Potassium (K⁺) channels are ubiquitous proteins which are involved in the setting of the resting membrane potential as well as in the modulation of the electrical activity of cells. In excitable cells, K⁺ channels influence action potential waveforms, firing frequency, and neurotransmitter secretion (Rudy, B. (1988) Neuroscience, 25, 729-749; Hille, B. (1992) Ionic Channels of Excitable Membranes, 2nd Ed.). In non-excitable cells, they are involved in hormone secretion, cell volume regulation and potentially in cell proliferation and differentiation (Lewis et al. (1995) Annu. Rev. Immunol., 13, 623-653). Developments in electrophysiology have allowed the identification and the characterization of an astonishing variety of K⁺ channels that differ in their biophysical properties, pharmacology, regulation and tissue distribution (Rudy, B. (1988) Neuroscience, 25, 729-749; Hille, B. (1992) Ionic Channels of Excitable Membranes, 2nd Ed.). More recently, cloning efforts have shed considerable light on the mechanisms that determine this functional diversity. Furthermore, analyses of structure-function relationships have provided an important set of data concerning the molecular basis of the biophysical properties (selectivity, gating, assembly) and the pharmacological properties of cloned K⁺ channels.

Functional diversity of K⁺ channels arises mainly from the existence of a great number of genes coding for pore-forming subunits, as well as for other associated regulatory subunits. Two main structural families of pore-forming subunits have been identified. The first one consists of subunits with a conserved hydrophobic core containing six transmembrane domains (TMDs). These K⁺ channel α subunits participate in the formation of outward rectifier voltage-gated (Kv) and Ca²⁺-dependent K⁺ channels. The fourth TMD contains repeated positive charges involved in the voltage gating of these channels and hence in their outward rectification (Logothetis et al. (1992) Neuron, 8, 531-540; Bezanilla et al. (1994) Biophys. J. 66, 1011-1021).

The second family of pore-forming subunits have only two TMDs. They are essential subunits of inward-rectifying (IRK), G-protein-coupled (GIRK) and ATP-sensitive (K_{ATP}) K⁺ channels. The inward rectification results from a voltage-dependent block by cytoplasmic Mg²⁺ and polyamines (Matsuda, H. (1991) Annu. Rev. Physiol., 53, 289-298). A conserved domain, called the P domain, is present in all members of both families (Pongs, O. (1993) J. Membr. Biol., 136, 1-8; Heginbotham et al. (1994) Biophys. J. 66,1061-1067; Mackinnon, R. (1995) Neuron, 14, 889-892; Pascual et al., (1995) Neuron., and 14, 1055-1063). This domain is an essential element of the aqueous K⁺-selective pore. In both groups, the assembly of four subunits is necessary to form a functional K⁺ channel (Mackinnon, R. (1991) Nature, 350, 232-235; Yang et al., (1995) Neuron, 15, 1441-1447.

In both six TMD and two TMD pore-forming subunit families, different subunits coded by different genes can associate to form heterotetramers with new channel properties (Isacoff et al., (1990) Nature, 345, 530-534). A selective formation of heteropolymeric channels may allow each cell to develop the best K⁺ current repertoire suited to its function. Pore-forming α subunits of Kv channels are classified into different subfamilies according to their sequence similarity (Chandy et al. (1993) Trends Pharmacol. Sci., 14: 434). Tetramerization is believed to occur preferentially between members of each subgroup (Covarrubias et al. (1991) Neuron, 7, 763-773). The domain responsible for this selective association is localized in the N-terminal region and is conserved between members of the same subgroup. This domain is necessary for hetero- but not homo-multimeric assembly within a subfamily and prevents co-assembly between subfamilies. Recently, pore-forming subunits with two TMDs were also shown to co-assemble to form heteropolymers (Duprat et al. (1995) Biochem. Biophys. Res. Commun., 212, 657-663. This heteropolymerization seems necessary to give functional GIRKs. IRKs are active as homopolymers but also form heteropolymers.

New structural types of K⁺ channels were identified recently in both humans and yeast. These channels have two P domains in their functional subunit instead of only one (Ketchum et al. (1995) Nature, 376, 690-695; Lesage et al. (1996) J. Biol. Chem., 271, 4183-4187; Lesage et al. (1996) EMBO J., 15, 1004-1011; Reid et al. (1996) Receptors Channels 4, 51-62). The human channel called TWIK-1, has four TMDs. TWIK-1 is expressed widely in human tissues and is particularly abundant in the heart and the brain. TWIK-1 currents are time independent and inwardly rectifying. These properties suggest that TWIK-1 channels are involved in the control of the background K⁺ membrane conductance (Lesage et al. (1996) EMBO J., 15, 1004-1011).

Potassium channels are potassium ion selective, and can determine membrane excitability (the ability of, for example, a neuron to respond to a stimulus and convert it into an impulse). Potassium channels can also influence the resting potential of membranes, wave forms and frequencies of action potentials, and thresholds of excitation. Potassium channels are typically expressed in electrically excitable cells, e.g., neurons, muscle, endocrine, and egg cells, and may form heteromultimeric structures, e.g., composed of pore-forming and cytoplasmic subunits. Potassium channels may also be found in non-excitable cells, where they may play a role in, e.g., signal transduction. Examples of potassium channels include: (1) the voltage-gated potassium channels, (2) the ligand-gated potassium channels, e.g., neurotransmitter-gated potassium channels, and (3) cyclic-nucleotide-gated potassium channels. Voltage-gated and ligand-gated potassium channels are expressed in the brain, e.g., in brainstem monoaminergic and forebrain cholinergic neurons, where they are involved in the release of neurotransmitters, or in the dendrites of hippocampal and neocortical pyramidal cells, where they are involved in the processes of learning and memory formation. For a detailed description of potassium channels, see Kandel E. R. et al., Principles of Neural Science, second edition, (Elsevier Science Publishing Co., Inc., N.Y. (1985)), the contents of which are incorporated herein by reference.

The E1-E2 ATPase family is a large superfamily of transport enzymes that contains at least 80 members found in diverse organisms such as bacteria, archaea, and eukaryotes (Palmgren, M. G. and Axelsen, K. B. (1998) Biochim. Biophys. Acta. 1365:37-45). These enzymes are involved in ATP hydrolysis-dependent transmembrane movement of a variety of inorganic cations (e.g., H⁺, Na⁺, K⁺, Ca²⁺, Cu²⁺, Cd⁺, and Mg²⁺ ions) across a concentration gradient, whereby the enzyme converts the free energy of ATP hydrolysis into electrochemical ion gradients. E1-E2 ATPases are also known as “P-type” ATPases, referring to the existence of a covalent high-energy phosphoryl-enzyme intermediate in the chemical reaction pathway of these transporters. Until recently, the superfamily contained four major groups: Ca²⁺ transporting ATPases; Na⁺/K⁺- and gastric H⁺/K⁺ transporting ATPases; plasma membrane H⁺ transporting ATPases of plants, fungi, and lower eukaryotes; and all bacterial P-type ATPases (Kuhlbrandt et al. (1998) Curr. Opin. Struct. Biol. 8:510-516).

E1-E2 ATPases are phosphorylated at a highly conserved DKTG sequence. Phosphorylation at this site is thought to control the enzyme's substrate affinity. Most E1-E2 ATPases contain ten alpha-helical transmembrane domains, although additional domains may be present. A majority of known gated-pore translocators contain twelve alpha-helices, including Na⁺/H⁺ antiporters (West (1997) Biochim. Biophys. Acta 1331:213-234).

Members of the E1-E2 ATPase superfamily are able to generate electrochemical ion gradients which enable a variety of processes in the cell such as absorption, secretion, transmembrane signaling, nerve impulse transmission, excitation/contraction coupling, and growth and differentiation (Scarborough (1999) Curr. Op. Cell Biol. 11:517-522). These molecules are thus critical to normal cell function and well-being of the organism.

Recently, a new class of E1-E2 ATPases was identified, the aminophospholipid transporters or translocators. These transporters transport not cations, but phospholipids (Tang, X. et al. (1996) Science 272:1495-1497; Bull, L. N. et al. (1998) Nat. Genet. 18:219-224; Mauro, I. et al. (1999) Biochem. Biophys. Res. Commun. 257:333-339). These transporters are involved in cellular functions including bile acid secretion and maintenance of the asymmetrical integrity of the plasma membrane.

Given the important biological and physiological roles played by the sugar transporter family of proteins, the potassium channel family of proteins, and the E1-E2 ATPase family of proteins, there exists a need to identify novel potassium channel family members for use in a variety of diagnostic/prognostic, as well as therapeutic applications

### SUMMARY OF THE INVENTION

The present invention is based, at least in part, on the discovery of novel human sugar transporter family members, referred to herein as “8099 and 46455” nucleic acid and polypeptide molecules. The 8099 and 46455 nucleic acid and polypeptide molecules of the present invention are useful as modulating agents in regulating a variety of cellular processes, e.g., sugar homeostasis. Accordingly, in one aspect, this invention provides isolated nucleic acid molecules encoding 8099 and 46455 polypeptides or biologically active portions thereof, as well as nucleic acid fragments suitable as primers or hybridization probes for the detection of 8099 and 46455-encoding nucleic acids.

The present invention is also based, at least in part, on the discovery of novel potassium channel family members, referred to herein as “54414 and 53763” nucleic acid and polypeptide molecules. The 54414 and 53763 nucleic acid and protein molecules of the present invention are useful as modulating agents in regulating a variety of cellular processes, e.g., gene expression, intra- or intercellular signaling, and/or membrane excitability or conductance. Accordingly, in one aspect, this invention provides isolated nucleic acid molecules encoding 54414 and 53763 proteins or biologically active portions thereof, as well as nucleic acid fragments suitable as primers or hybridization probes for the detection of 54414 and 53763-encoding nucleic acids.

The present invention is also based, at least in part, on the discovery of novel human phospholipid transporter family members, referred to herein as “67076, 67102, 44181, 67084FL, or 67084alt” nucleic acid and polypeptide molecules. The 67076, 67102, 44181, 67084FL, or 67084alt nucleic acid and polypeptide molecules of the present invention are useful as modulating agents in regulating a variety of cellular processes, e.g., phospholipid transport (e.g., aminophospholipid transport), absorption, secretion, gene expression, intra- or inter-cellular signaling, and/or cellular proliferation, growth, apoptosis, and/or differentiation. Accordingly, in one aspect, this invention provides isolated nucleic acid molecules encoding 67076, 67102, 44181, 67084FL, or 67084alt polypeptides or biologically active portions thereof, as well as nucleic acid fragments suitable as primers or hybridization probes for the detection of 67076, 67102, 44181, 67084FL, or 67084alt-encoding nucleic acids.

In one embodiment, the invention features an isolated nucleic acid molecule that includes the nucleotide sequence set forth in SEQ ID NO:46, 48, 49, 51, 52, 54, 55, 57, 58, 60, 61, 63, 64, 66, 67, 69, 70, or 72. In another embodiment, the invention features an isolated nucleic acid molecule that encodes a polypeptide including the amino acid sequence set forth in SEQ ID NO:47, 50, 53, 56, 59, 62, 65, 68, or 71. In another embodiment, the invention features an isolated nucleic acid molecule that includes the nucleotide sequence contained in the plasmid deposited with ATCC® as Accession Number ______, ______, ______, ______, or ______.

In still other embodiments, the invention features isolated nucleic acid molecules including nucleotide sequences that are substantially identical (e.g., 60% identical) to the nucleotide sequence set forth as SEQ ID NO:46, 48, 49, 51, 52, 54, 55, 57, 58, 60, 61, 63, 64, 66, 67, 69, 70, or 72. The invention further features isolated nucleic acid molecules including at least 50 contiguous nucleotides of the nucleotide sequence set forth as SEQ ID NO:46, 48, 49, 51, 52, 54, 55, 57, 58, 60, 61, 63, 64, 66, 67, 69, 70, or 72. In another embodiment, the invention features isolated nucleic acid molecules which encode a polypeptide including an amino acid sequence that is substantially identical (e.g., 60% identical) to the amino acid sequence set forth as SEQ ID NO:47, 50, 53, 56, 59, 62, 65, 68, or 71. The present invention also features nucleic acid molecules which encode allelic variants of the polypeptide having the amino acid sequence set forth as SEQ ID NO:47, 50, 53, 56, 59, 62, 65, 68, or 71. In addition to isolated nucleic acid molecules encoding full-length polypeptides, the present invention also features nucleic acid molecules which encode fragments, for example, biologically active or antigenic fragments, of the full-length polypeptides of the present invention (e.g., fragments including at least 10 contiguous amino acid residues of the amino acid sequence of SEQ ID NO:47, 50, 53, 56, 59, 62, 65, 68, or 71). In still other embodiments, the invention features nucleic acid molecules that are complementary to, antisense to, or hybridize under stringent conditions to the isolated nucleic acid molecules described herein.

In another aspect, the invention provides vectors including the isolated nucleic acid molecules described herein (e.g., 8099, 46455, 54414, 53763, 67076, 67102, 44181, 67084FL, or 67084alt-encoding nucleic acid molecules). Such vectors can optionally include nucleotide sequences encoding heterologous polypeptides. Also featured are host cells including such vectors (e.g., host cells including vectors suitable for producing 8099, 46455, 54414, 53763, 67076, 67102, 44181, 67084FL, or 67084alt nucleic acid molecules and polypeptides).

In another aspect, the invention features isolated 8099, 46455, 54414, 53763, 67076, 67102, 44181, 67084FL, or 67084alt polypeptides and/or biologically active or antigenic fragments thereof. Exemplary embodiments feature a polypeptide including the amino acid sequence set forth as SEQ ID NO:47, 50, 53, 56, 59, 62, 65, 68, or 71, a polypeptide including an amino acid sequence at least 60% identical to the amino acid sequence set forth as SEQ ID NO:47, 50, 53, 56, 59, 62, 65, 68, or 71, a polypeptide encoded by a nucleic acid molecule including a nucleotide sequence at least 60% identical to the nucleotide sequence set forth as SEQ ID NO:46, 48, 49, 51, 52, 54, 55, 57, 58, 60, 61, 63, 64, 66, 67, 69, 70, or 72. Also featured are fragments of the full-length polypeptides described herein (e.g., fragments including at least 10 contiguous amino acid residues of the sequence set forth as SEQ ID NO:47, 50, 53, 56, 59, 62, 65, 68, or 71) as well as allelic variants of the polypeptide having the amino acid sequence set forth as SEQ ID NO:47, 50, 53, 56, 59, 62, 65, 68, or 71.

The 8099 and 46455 polypeptides and/or biologically active or antigenic fragments thereof, are useful, for example, as reagents or targets in assays applicable to treatment and/or diagnosis of 8099 and 46455 mediated or related disorders. In one embodiment, 8099 and/or 46455 polypeptides or fragments thereof, have an 8099 and/or 46455 activity. In another embodiment, 8099 and/or 46455 polypeptides or fragments thereof, have at least one, preferably two, three, four, five, six, seven, eight, nine, ten, eleven, or twelve transmembrane domains and/or a sugar transporter family domain, and optionally, have an 8099 and/or 46455 activity.

The 54414 and 53763 polypeptides and/or biologically active or antigenic fragments thereof, are useful, for example, as reagents or targets in assays applicable to treatment and/or diagnosis of 54414 and 53763 mediated or related disorders. In one embodiment, a 54414 AND 53763 polypeptide or fragment thereof has a 54414 and 53763 activity. In another embodiment, a 54414 and 53763 polypeptide or fragment thereof has at least one or more of the following domains or motifs: a transmembrane domain, an ion transport protein domain, a K⁺ channel tetramerization domain, a P-loop motif, a pore domain, a potassium channel signature sequence motif, and/or a voltage sensor motif, and optionally, has a 54414 or 53763 activity.

The 67076, 67102, 44181, 67084FL, or 67084alt polypeptides and/or biologically active or antigenic fragments thereof, are useful, for example, as reagents or targets in assays applicable to treatment and/or diagnosis of 67076, 67102, 44181, 67084FL, or 67084alt associated or related disorders. In one embodiment, a 67076, 67102, 44181, 67084FL, or 67084alt polypeptide or fragment thereof, has a 67076, 67102, 44181, 67084FL, or 67084alt activity. In another embodiment, a 67076, 67102, 44181, 67084FL, or 67084alt polypeptide or fragment thereof, includes at least one of the following domains, sites, or motifs: a transmembrane domain, an N-terminal large extramembrane domain, a C-terminal large extramembrane domain, an E1-E2 ATPases phosphorylation site, a P-type ATPase sequence 1 motif, a P-type ATPase sequence 2 motif, a P-type ATPase sequence 3 motif, and/or one or more phospholipid transporter specific amino acid resides, and optionally, has a 67076, 67102, 44181, 67084FL, or 67084alt activity.

In a related aspect, the invention features antibodies (e.g., antibodies which specifically bind to any one of the polypeptides described herein) as well as fusion polypeptides including all or a fragment of a polypeptide described herein.

The present invention further features methods for detecting 8099, 46455, 54414, 53763, 67076, 67102, 44181, 67084FL, or 67084alt polypeptides and/or 8099, 46455, 54414, 53763, 67076, 67102, 44181, 67084FL, or 67084alt nucleic acid molecules, such methods featuring, for example, a probe, primer or antibody described herein. Also featured are kits, e.g., kits for the detection of 8099, 46455, 54414, 53763, 67076, 67102, 44181, 67084FL, or 67084alt polypeptides and/or 8099, 46455, 54414, 53763, 67076, 67102, 44181, 67084FL, or 67084alt nucleic acid molecules. In a related aspect, the invention features methods for identifying compounds which bind to and/or modulate the activity of a 8099, 46455, 54414, 53763, 67076, 67102, 44181, 67084FL, or 67084alt polypeptide or 8099, 46455, 54414, 53763, 67076, 67102, 44181, 67084FL, or 67084alt nucleic acid molecule described herein. Further featured are methods for modulating a 67076, 67102, 44181, 67084FL, or 67084alt activity.

Other features and advantages of the invention will be apparent from the following detailed description and claims.

### Detailed Description of the Invention

The present invention is based, at least in part, on the discovery of novel sugar transporter family molecules, referred to herein as “8099 and 46455” nucleic acid and polypeptide molecules. These novel molecules are capable of, for example, modulating a transporter mediated activity (e.g., a sugar transporter mediated activity) in a cell, e.g., a liver cell, fat cell, muscle cell, or blood cell, such as an erythrocyte. These novel molecules are capable of transporting molecules, e.g., hexoses such as D-glucose, D-fructose, D-galactose or mannose across biological membranes and, thus, play a role in or function in a variety of cellular processes, e.g., maintenance of sugar homeostasis. Thus the 8099 and 46455 molecules of the present invention provide novel diagnostic targets and therapeutic agents to control 8099 and 46455-associated disorders, as defined herein.

The present invention is also based, at least in part, on the discovery of novel potassium channel family members, referred to herein as “54414 and 53763” nucleic acid and polypeptide molecules. These novel molecules are capable of, for example, modulating PCH mediated activities in a cell, e.g., a neuronal cell. Thus, the 54414 and 53763 molecules of the present invention provide novel diagnostic targets and therapeutic agents to control 54414 or 53763-associated disorders, as defined herein.

The present invention also is based, at least in part, on the discovery of novel phospholipid transporter family molecules, referred to herein as “67076, 67102, 44181, 67084FL, or 67084alt” nucleic acid and polypeptide molecules. These novel molecules are capable of, for example, transporting phospholipids (e.g., aminophospholipids such as phosphatidylserine and phosphatidylethanolamine, choline phospholipids such as phosphatidylcholine and sphingomyelin, and bile acids) across cellular membranes and, thus, play a role in or function in a variety of cellular processes, e.g., phospholipid transport, absorption, secretion, gene expression, intra- or inter-cellular signaling, and/or cellular proliferation, growth, and/or differentiation. Thus, the 67076, 67102, 44181, 67084FL, and 67084alt molecules of the present invention provide novel diagnostic targets and therapeutic agents to control 67076, 67102, 44181, 67084FL, or 67084alt-associated disorders, as defined herein.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin or alternatively, can contain homologues of non-human origin, e.g., rat or mouse proteins. Members of a family can also have common functional characteristics.

8099 and 46455 Molecules of the Invention

The family of 8099 and 46455 polypeptides comprise at least one “transmembrane domain” and at least one, preferably two, three, four, five, six, seven, eight, nine, ten, eleven, or twelve transmembrane domains. As used herein, the term “transmembrane domain” includes an amino acid sequence of about 20-45 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, alanines, valines, phenylalanines, prolines or methionines. Transmembrane domains are described in, for example, Zagotta W. N. et al, (1996) Annual Rev. Neurosci. 19: 235-263, the contents of which are incorporated herein by reference. A MEMSAT and additional analyses resulted in the identification of twelve transmembrane domains in the amino acid sequence of human 8099 (SEQ ID NO:47) at about residues 32-49, 81-101, 109-130, 138-156, 165-184, 198-217, 279-301, 315-338, 346-364, 463-487, 499-521, and 529-549. A MEMSAT and additional analyses resulted in the identification of twelve transmembrane domains in the amino acid sequence of human 46455 (SEQ ID NO:50) at about residues 58-74, 98-118, 126-145, 165-181, 188-205, 218-238, 273-294, 323-341, 357-377, 386-410, 423-441, and 462-485.

Accordingly, 8099 and 46455 polypeptides having at least 50-60% homology, preferably about 60-70%, more preferably about 70-80%, or about 80-90% homology with at least one, preferably at least two, three, four, five, six, seven, eight, nine, ten, eleven, or twelve transmembrane domains of human 8099 and 46455, respectively are within the scope of the invention.

Another embodiment of the invention features 8099 molecules which contain an N-terminal unique domain. The term “unique N-terminal domain” as used herein, refers to a protein domain of an 8099 protein family member which includes amino acid residues N-terminal to the sixth transmembrane domain, e.g., the GLUT8-like domain in the amino acid sequence of the 8099 protein. As used herein, a “unique N-terminal domain” refers to a protein domain which is at least about 150-200 amino acid residues in length, preferably at least about 160-190 amino acid residues in length and shares significantly more sequence homology with about residues 1 to 178 of SEQ ID NO:47 than with about residues 1 to 178 of GLUT8.

Accordingly, 8099 polypeptides having at least 50-60% homology, preferably about 60-70%, more preferably about 70-80%, or about 80-90% homology with at least one unique N-terminal domain of human 8099 (e.g., about amino acids 1-178 of SEQ ID NO:47) are within the scope of the invention.

Yet another aspect of the invention features 8099 proteins having an “extended exofacial loop” between transmembrane domains 9 and 10. Preferably, the first amino acid residue of an extended exofacial loop of 8099 is the first residue C-terminal to the amino acid residues of transmembrane domain 9 and the last residue of the exofacial loop is the first residue N-terminal to the amino acid residues of transmembrane domain 10 of 8099. In a preferred embodiment, an extended exofacial loop is at least about 50-55, 55-60, 60-65, 65-70, 70-75, 75-80, 80-85, 85-90, 90-95, 97 or more amino acid residues in length. For example, in one embodiment, an 8099 protein includes an “extended exofacial loop” of about amino acids 365-462 of SEQ ID NO:47 (97 amino acid residues in length).

Accordingly, 8099 polypeptides having at least 50-60% homology, preferably about 60-70%, more preferably about 70-80%, or about 80-90% homology with at least one extended exofacial loop of human 8099 are within the scope of the invention.

In another embodiment, an 8099 and/or 46455 molecule of the present invention is identified based on the presence of at least one “sugar transporter family domain.” As used herein, the term “sugar transporter family domain” includes a protein domain having at least about 300-600 amino acid residues and a sugar transporter mediated activity. Preferably, a sugar transporter family domain includes a polypeptide having an amino acid sequence of about 350-550, 400-550, or more preferably, about 411 or 521 amino acid residues and a sugar transporter mediated activity. To identify the presence of a sugar transporter family domain in an 8099 and/or an 46455 protein, and make the determination that a protein of interest has a particular profile, the amino acid sequence of the protein may be searched against a database of known protein domains (e.g., the PFAM HMM database). A PFAM sugar transporter family domain has been assigned the PFAM Accession PF00083. A search was performed against the PFAM HMM database resulting in the identification of a sugar transporter family domain in the amino acid sequence of human 8099 (SEQ ID NO:47) at about residues 43-564 of SEQ ID NO:47. A search was performed against the PFAM HMM database resulting in the identification of a sugar transporter family domain in the amino acid sequence of human 46455 (SEQ ID NO:50) at about residues 58-487 of SEQ ID NO:50.

Preferably a “sugar transporter family domain” has a “sugar transporter mediated activity” as described herein. For example, a sugar transporter family domain may have the ability to bind a monosaccharide (e.g., D-glucose, D-fructose, D-galactose and/or mannose); the ability to transport a monosaccharide (e.g., D-glucose, D-fructose, D-galactose and/or mannose) in a constitutive manner or in response to stimuli (e.g., insulin) across a cell membrane (e.g., a liver cell membrane, fat cell membrane, muscle cell membrane, and/or blood cell membrane, such as an erythrocyte membrane); the ability to function as a neuronal transporter; the ability to mediate trans-epithelial movement; and/or the ability to modulate sugar homeostasis in a cell. Accordingly, identifying the presence of a “sugar transporter family domain” can include isolating a fragment of an 8099 and/or an 46455 molecule (e.g., an 8099 and/or an 46455 polypeptide) and assaying for the ability of the fragment to exhibit one of the aforementioned sugar transporter mediated activities.

A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al.(1994) J. Mol. Biol. 235:1501-1531; and Stultz et al.(1993) Protein Sci. 2:305-314, the contents of which are incorporated herein by reference.

In a preferred embodiment, the 8099 and/or 46455 molecules of the invention include at least one, preferably two, even more preferably at least three, four, five, six, seven, eight, nine, ten, eleven, or twelve transmembrane domain(s) and/or at least one sugar transporter family domain. In another preferred embodiment, the 8099 molecules of the invention include at least one, preferably two, even more preferably at least three, four, five, six, seven, eight, nine, ten, eleven, or twelve transmembrane domain(s), at least one sugar transporter family domain, at least one unique N-terminal domain, and/or at least one extended exofacial loop.

Isolated polypeptides of the present invention, preferably 8099 or 46455 polypeptides, have an amino acid sequence sufficiently identical to the amino acid sequence of SEQ ID NO:47 or 50 or are encoded by a nucleotide sequence sufficiently identical to SEQ ID NO: SEQ ID NO:46, 48, 49, or 51. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains having at least 50%, 55%, 60%, 65%, 70%, 75%, 80%, 85%, 85%, 90%, 95%, 96%, 97%, 98%, 99% or more homology or identity across the amino acid sequences of the domains and contain at least one and preferably two structural domains or motifs, are defined herein as sufficiently identical. Furthermore, amino acid or nucleotide sequences which share at least 50%, 55%, 60%, 65%, 70%, 75%, 80%, 85%, 85%, 90%, 95%, 96%, 97%, 98%, 99% or more homology or identity and share a common functional activity are defined herein as sufficiently identical.

In a preferred embodiment, an 8099 and/or 46455 polypeptide includes at least one or more of the following domains: a transmembrane domain and/or a sugar transporter family domain, and has an amino acid sequence at least about 50%, 55%, 60%, 65%, 70%, 75%, 80%, 85%, 85%, 90%, 95%, 96%, 97%, 98%, 99% or more homologous or identical to the amino acid sequence of SEQ ID NO:47 or 50, or the amino acid sequences encoded by the DNA inserts of the plasmids deposited with ATCC as Accession Numbers ______ and/or ______. In yet another preferred embodiment, an 8099 and/or an 46455 polypeptide includes at least one or more of the following domains: a transmembrane domain and/or a sugar transporter family domain, and is encoded by a nucleic acid molecule having a nucleotide sequence which hybridizes under stringent hybridization conditions to a complement of a nucleic acid molecule comprising the nucleotide sequence of SEQ ID NO:46, 48, 49, or 51. In another preferred embodiment, an 8099 and/or an 46455 polypeptide includes at least one or more of the following domains: a transmembrane domain and/or a sugar transporter family domain, and has an 8099 and/or an 46455 activity.

As used interchangeably herein, an “8099 activity”, “46455 activity”, “biological activity of 8099”, “biological activity of 46455”, “functional activity of 8099” or “functional activity of 46455” refers to an activity exerted by an 8099 and/or 46455 polypeptide or nucleic acid molecule on an 8099 and/or 46455 responsive cell or tissue, or on an 8099 and/or 46455 polypeptide substrate, as determined in vivo, or in vitro, according to standard techniques. In one embodiment, an 8099 and/or 46455 activity is a direct activity, such as an association with an 8099- and/or 46455-target molecule. As used herein, a “substrate,” “target molecule,” or “binding partner” is a molecule with which an 8099 and/or 46455 polypeptide binds or interacts in nature, such that 8099- and/or 46455-mediated function is achieved. An 8099 and/or 46455 target molecule can be a non-8099 and/or a non-46455 molecule or an 8099 and/or 46455 polypeptide or polypeptide of the present invention. In an exemplary embodiment, an 8099 and/or 46455 target molecule is an 8099 and/or 46455 ligand, e.g., a sugar transporter ligand such D-glucose, D-fructose, D-galactose, and/or mannose. Alternatively, an 8099 and/or 46455 activity is an indirect activity, such as a cellular signaling activity mediated by interaction of the 8099 and/or 46455 polypeptide with an 8099 and/or 46455 ligand. The biological activities of 8099 and/or 46455 are described herein. For example, the 8099 and/or 46455 polypeptides of the present invention can have one or more of the following activities: (1) bind a monosaccharide, e.g., D-glucose, D-fructose, D-galactose, and/or mannose, (2) transport monosaccharides across a cell membrane, (3) influence insulin and/or glucagon secretion, (4) maintain sugar homeostasis in a cell, (5) function as a neuronal transporter, and (6) mediate trans-epithelial movement in a cell. Moreover, in a preferred embodiment, 8099 and/or 46455 molecules of the present invention, 8099 and/or 46455 antibodies, 8099 and/or 46455 modulators are useful in at least one of the following: (1) modulation of insulin sensitivity; (2) modulation of blood sugar levels; (3) treatment of blood sugar level disorders (e.g., diabetes); and/or (4) modulation of insulin resistance.

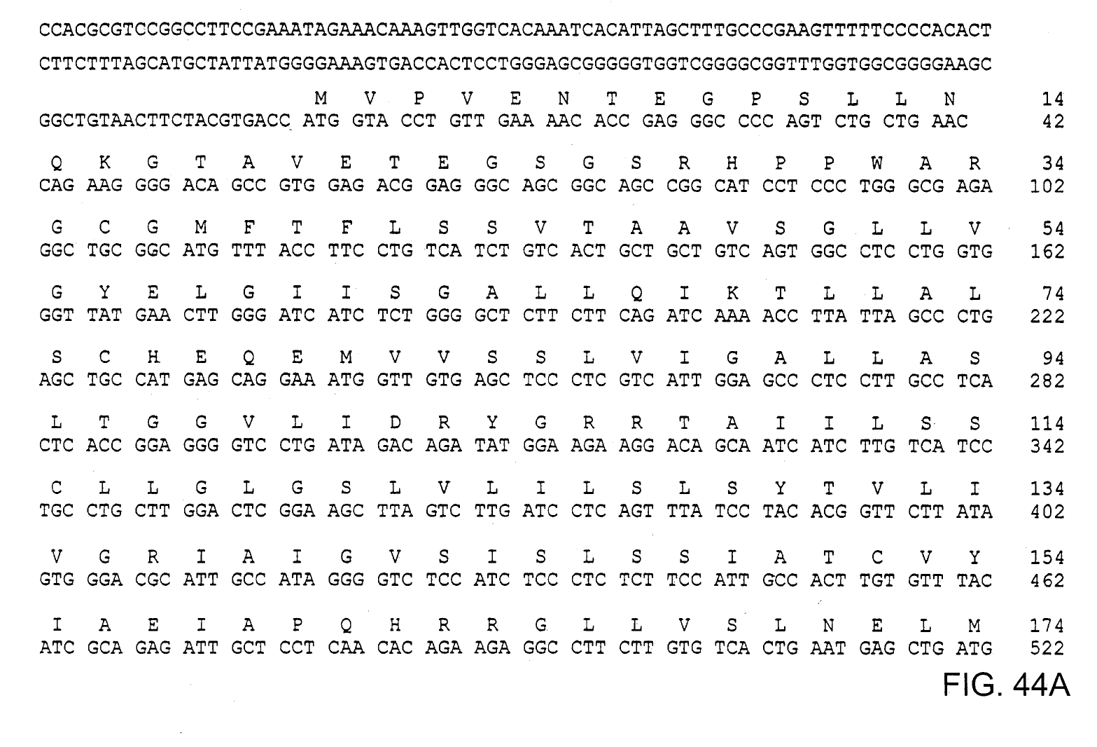

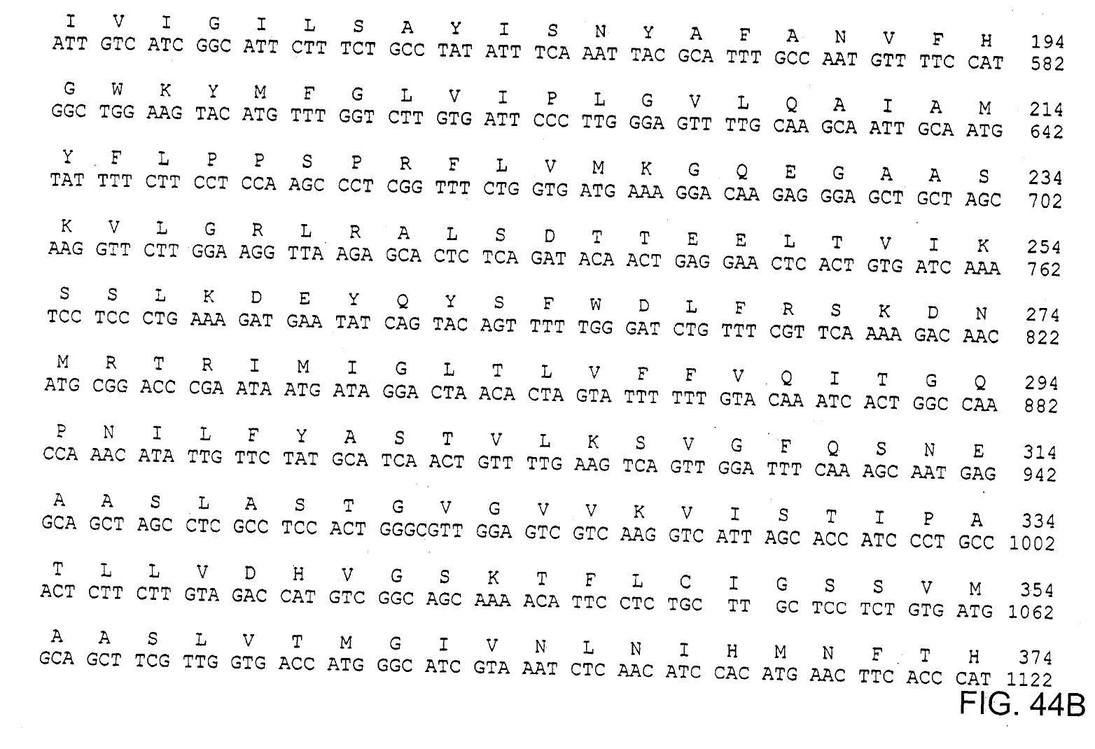

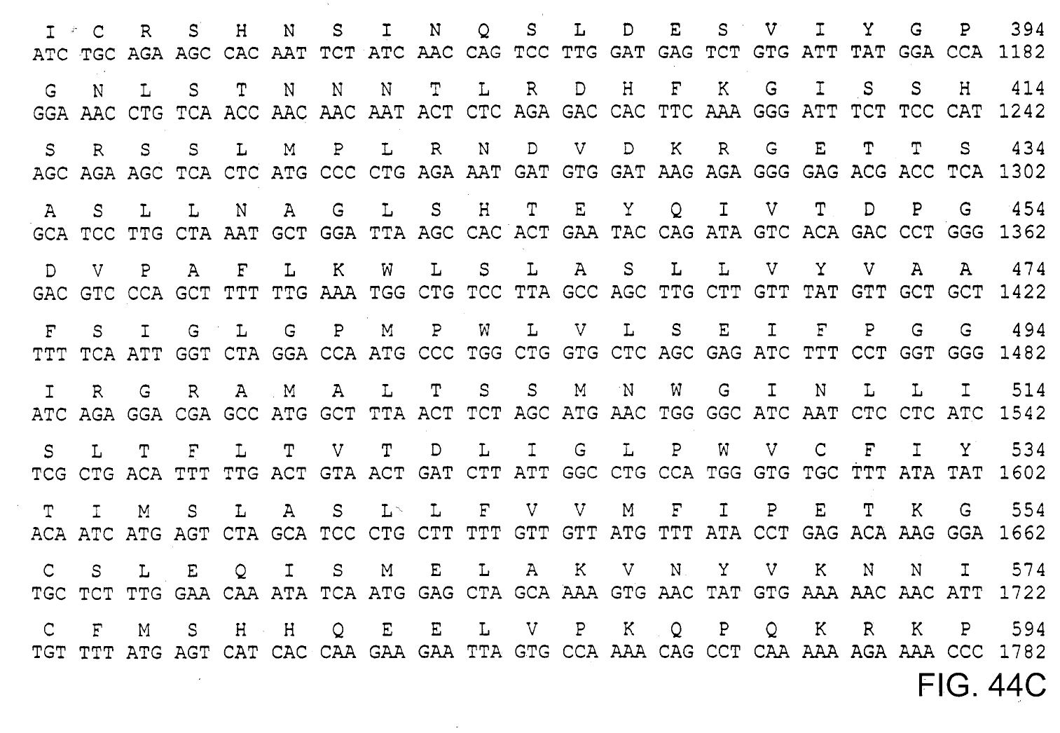

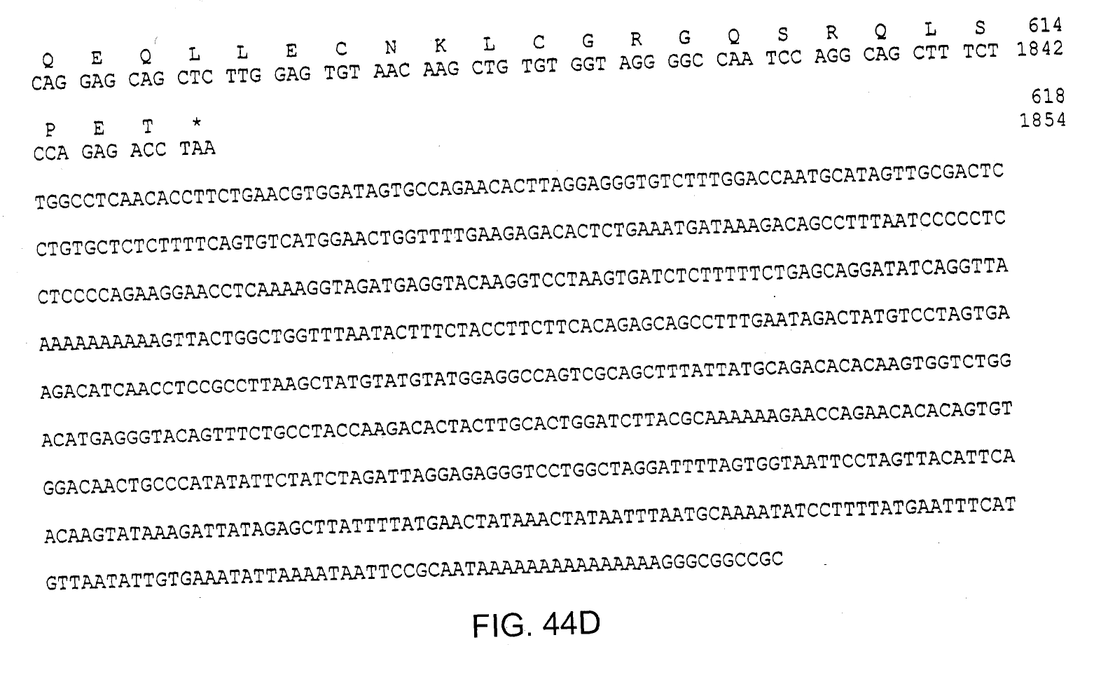

The nucleotide sequence of the isolated human 8099 and 46455 cDNAs and the predicted amino acid sequences of the human 8099 and 46455 polypeptides are shown in FIGS. 44A-D and 51A-D and in SEQ ID NOs:46 and 47, and SEQ ID NOs:49 and 50, respectively. Plasmids containing the nucleotide sequences encoding human 8099 or 46455 were deposited with the American Type Culture Collection (ATCC), 10801 University Boulevard, Manassas, Va. 20110-2209, on ______ and assigned Accession Numbers ______ or ______. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112.

The human 8099 gene, which is approximately 2725 nucleotides in length, encodes a polypeptide which is approximately 617 amino acid residues in length. The human 46455 gene, which is approximately 2230 nucleotides in length, encodes a polypeptide which is approximately 528 amino acid residues in length.

54414 and 53763 Molecules of the Invention

The family of 54414 and 53763 proteins of the present invention comprises at least one transmembrane domain, preferably at least 2 or 3 transmembrane domains, more preferably 4 or 5 transmembrane domains, and most preferably, 6 transmembrane domains. Amino acid residues 64-83, 104-127, 135-153, 161-173, 199-217, and 257-274 of the human 54414 protein (SEQ ID NO:53) are predicted to comprise transmembrane domains. Amino acid residues 230-248, 287-303, 314-335, 346-368, 382-402, and 451-473 of the human 53763 protein (SEQ ID NO:56) are predicted to comprise transmembrane domains.

In another embodiment, members of the 54414 and 53763 family of proteins include at least one “ion transport protein domain” in the protein or corresponding nucleic acid molecule. As used herein, the term “ion transport protein domain” includes a protein domain having at least about 150-310 amino acid residues and a bit score of at least 200 when compared against an ion transport protein domain Hidden Markov Model (HMM), e.g., PFAM Accession Number PF00520. Preferably, an ion transport protein domain includes a protein domain having an amino acid sequence of about 170-290, 190-270, 210-250, or more preferably about 173 or 191 amino acid residues. To identify the presence of an ion transport protein domain in a 54414 or 53763 protein, and make the determination that a protein of interest has a particular profile, the amino acid sequence of the protein is searched against a database of known protein motifs and/or domains (e.g., the HMM database). The ion transport protein domain (HMM) has been assigned the PFAM Accession number PF00520. A search was performed against the HMM database resulting in the identification of an ion transport protein domain in the amino acid sequence of human 54414 at about residues 104-277 of SEQ ID NO:53 and in the amino acid sequence of human 53763 about residues 281-472 of SEQ ID NO:56.

Preferably an ion transport protein domain is at least about 150-310 amino acid residues and has an “ion transport protein domain activity”, for example, the ability to interact with a 54414 or 53763 substrate or target molecule (e.g., a potassium ion) and/or to regulate 54414 or 53763 activity. Accordingly, identifying the presence of an “ion transport protein domain” can include isolating a fragment of a 54414 or 53763 molecule (e.g., a 54414 or 53763 polypeptide) and assaying for the ability of the fragment to exhibit one of the aforementioned ion transport protein domain activities.

In another embodiment, members of the 54414 and 53763 family of proteins include at least one “K⁺ channel tetramerization domain” in the protein or corresponding nucleic acid molecule. As used herein, the term “K⁺ channel tetramerization domain” includes a protein domain having at least about 70-230 amino acid residues and a bit score of at least 80 when compared against a K⁺ channel tetramerization domain Hidden Markov Model (HMM), e.g., PFAM Accession Number PF02214. Preferably, a K⁺ channel tetramerization domain includes a protein domain having an amino acid sequence of about 90-210, 110-190, 130-170, or more preferably about 149 amino acid residues, and a bit score of at least 100, 120, 140, or more preferably, 156.7. To identify the presence of a K⁺ channel tetramerization domain in a 54414 or 53763 protein, and make the determination that a protein of interest has a particular profile, the amino acid sequence of the protein is searched against a database of known protein motifs and/or domains (e.g., the HMM database). The K⁺ channel tetramerization domain (HMM) has been assigned the PFAM Accession number PF02214. A search was performed against the HMM database resulting in the identification of a K⁺ channel tetramerization domain in the amino acid sequence of human 53763 at about residues 8-156 of SEQ ID NO:56.

Preferably a K⁺ channel tetramerization domain is at least about 70-230 amino acid residues and has an “K⁺ channel tetramerization domain activity”, for example, the ability to interact with one or more potassium channel subunits (e.g., 54414 or 53763 molecules, or non-54414 or 53763 potassium channel subunits), the ability to regulate assembly of a 54414 or 53763 molecule into a potassium channel tetramer, and/or to regulate 54414 or 53763 activity. Accordingly, identifying the presence of an “K⁺ channel tetramerization domain” can include isolating a fragment of a 54414 or 53763 molecule (e.g., a 54414 or 53763 polypeptide) and assaying for the ability of the fragment to exhibit one of the aforementioned K⁺ channel tetramerization domain activities.

In another embodiment, a 54414 or 53763 protein of the present invention is identified based on the presence of an “ATP/GTP-binding sit motif A (P-loop) motif”, referred to alternatively herein as a “P-loop motif”, in the protein or corresponding nucleic acid molecule. Preferably, a P-loop motif includes a protein motif which is about 4-15, 5-13, 6-11, 7-9, or preferably about 8 amino acid residues. The P-loop motif functions in binding ATP and/or GTP via interaction with the phosphate groups of the nucleotide and has been assigned Prosite™ Accession Number PS00017. To identify the presence of a P-loop motif in a 54414 or 53763 protein, and to make the determination that a protein of interest has a particular profile, the amino acid sequence of the protein may be searched against a database of known protein domains or motifs (e.g., the Prosite™ database) using the default parameters (available at the ProSite website). A search was performed against the ProSite database resulting in the identification of a P-loop motif in the amino acid sequence of human 54414 (SEQ ID NO:53) at about residues 1007-1014.

In another embodiment, a 54414 or 53763 protein of the present invention is identified based on the presence of a “pore domain”, alternatively referred to herein as a “P-region domain”, in the protein or corresponding nucleic acid molecule. As used interchangeably herein, the terms “pore domain” and “P-region domain” include a protein domain having about 10-30, 12-28, 13-25, 14-24, 15-23, or preferably about 16-22 amino acid residues, which is involved in lining the potassium channel pore. A pore domain is typically found between transmembrane domains of potassium channels and is believed to be a major determinant of ion selectivity in potassium channels. Preferably, a pore domain includes a potassium channel signature motif, as defined herein. Pore domains are described in, for example, Warmke et al. (1991) Science 252:1560-1562; Zagotta W. N. et al. (1996) Annu. Rev. Neurosci. 19:235-63; Pongs, O. (1993) J. Membr. Biol. 136:1-8; Heginbotham et al. (1994) Biophys. J. 66:1061-1067; Mackinnon, R. (1995) Neuron 14:889-892; and Pascual et al. (1995) Neuron 14:1055-1063), the contents of which are incorporated herein by reference. A pore domain was identified in the amino acid sequence of human 54414 at about residues 229-250 of SEQ ID NO:53. A pore domain was identified in the amino acid sequence of human 53763 at about residues 426-441 of SEQ ID NO:56.

In a further embodiment, a 54414 or 53763 protein of the present invention is identified based on the presence of a “potassium channel signature sequence motif” in the protein or corresponding nucleic acid molecule. As used herein, the term “potassium channel signature sequence motif” includes a protein motif which is diagnostic for potassium channels. Preferably, a potassium channel signature sequence motif has the consensus sequence T-X-X-T-X-G-hydrophobic-G (see Joiner, W. J. et al. (1998) Nat. Neurosci. 1:462-469 and references cited therein), wherein “X” indicates any amino acid residue, and “hydrophobic” indicates any hydrophobic amino acid residue. Preferably, a potassium channel signature sequence motif is included within a pore domain and includes at least 1, 2, 3, 4, 5, 6, 7, or more preferably, 8 amino acid residues that match the consensus sequence for a potassium channel signature sequence motif. A potassium channel signature sequence motif was identified in the amino acid sequence of human 54414 at about residues 239-246 of SEQ ID NO:53. A potassium channel signature sequence motif was identified in the amino acid sequence of human 53763 at about residues 436-441 of SEQ ID NO:56.

In still another embodiment, a 54414 or 53763 protein of the present invention is identified based on the presence of a “voltage sensor motif”, alternatively referred to simply as a “voltage sensor”, in the protein or the corresponding nucleic acid molecule. As used interchangeably herein, the terms “voltage sensor motif” and “voltage sensor” include a protein motif having about 10-30, 11-26, 12-24, 13-22, 14-20, 15-18, or more preferably 16 amino acid residues, which is involved in sensing voltage differences between the two sides of the plasma membrane of a cell. Preferably, a voltage sensor motif includes at least 1, 2, 3, 4, 5, or more preferably, 6 positively charged amino acid residues, which are preferably spaced apart by at least 1, or preferably 2, non-positively charged amino acid residues. Preferably, a voltage sensor motif is included within and/or overlaps with a transmembrane domain, more preferably the fourth transmembrane, of the 54414 or 53763 protein in which it is found. A voltage sensor motif was identified in the amino acid sequence of human 53763 at about residues 348-363 of SEQ ID NO:53. The positively charged amino acid residues of the human 53763 voltage sensor were identified at about residues 348, 351, 354, 357, 360, and 363 of SEQ ID NO:53. No voltage sensor was identified in human 54414.

Isolated proteins of the present invention, preferably 54414 or 53763 proteins, have an amino acid sequence sufficiently homologous to the amino acid sequence of SEQ ID NO:53 or SEQ ID NO:56, or are encoded by a nucleotide sequence sufficiently homologous to SEQ ID NO:52, 54, 55, or 57. Amino acid or nucleotide sequences which share common structural domains having at least 50%, 55%, 60%, 65%, 70%, 75%, 80%, 85%, 85%, 90% 95%, 96%, 97%, 98%, 99% or more homology or identity across the amino acid sequences of the domains and contain at least one and preferably two structural domains or motifs, are defined herein as sufficiently homologous. Furthermore, amino acid or nucleotide sequences which share at least 50%, 55%, 60%, 65%, 70%, 75%, 80%, 85%, 85%, 90%, 95%, 96%, 97%, 98%, 99% or more homology or identity and share a common functional activity are defined herein as sufficiently homologous.

In a preferred embodiment, a 54414 or 53763 protein includes at least one or more of the following domains or motifs: a transmembrane domain, an ion transport protein domain, a K⁺ channel tetramerization domain, a P-loop motif, a pore domain, a potassium channel signature sequence motif, and/or a voltage sensor motif. and has an amino acid sequence at least about 50%, 55%, 60%, 65%, 70%, 75%, 80%, 85%, 85%, 90%, 95% 96%, 97%, 98%, 99% or more homologous or identical to the amino acid sequence of SEQ ID NO:53 or 56, or the amino acid sequence encoded by the DNA insert of the plasmid deposited with ATCC as Accession Number ______ or _______. In yet another preferred embodiment, a 54414 or 53763 protein includes at least one or more of the following domains or motifs: a transmembrane domain, an ion transport protein domain, a K⁺ channel tetramerization domain, a P-loop motif, a pore domain, a potassium channel signature sequence motif, and/or a voltage sensor motif, and is encoded by a nucleic acid molecule having a nucleotide sequence which hybridizes under stringent hybridization conditions to a complement of a nucleic acid molecule comprising the nucleotide sequence of SEQ ID NO:52, 54, 55, or 57. In another preferred embodiment, a 54414 or 53763 protein includes at least one or more of the following domains or motifs: a transmembrane domain, an ion transport protein domain, a K⁺ channel tetramerization domain, a P-loop motif, a pore domain, a potassium channel signature sequence motif, and/or a voltage sensor motif, and has a 54414 or 53763 activity.

As used interchangeably herein, a “54414 or 53763 activity”, “biological activity of 54414 or 53763” or “functional activity of 54414 or 53763”, includes an activity exerted or mediated by a 54414 or 53763 protein, polypeptide or nucleic acid molecule when expressed in a cell or on a membrane, as determined in vivo or in vitro, according to standard techniques. In one embodiment, a 54414 or 53763 activity is a direct activity, such as transport of a 54414 or 53763 substrate (e.g., a potassium ion). In another embodiment, a 54414 or 53763 activity is an indirect activity mediated, for example, by interaction of a 54414 or 53763 molecule with a 54414 or 53763 target molecule or binding partner. As used herein, a “target molecule” or “binding partner” is a molecule with which a 54414 or 53763 protein binds or interacts in nature, such that function of the target molecule or binding partner is modulated. In an exemplary embodiment, a 54414 or 53763 target molecule or binding partner is a 54414 or 53763 polypeptide or a non-54414 or 53763 potassium channel subunit.

In a preferred embodiment, a 54414 or 53763 activity is at least one of the following activities: (i) interaction with a 54414 or 53763 substrate (e.g., a potassium ion or a cyclic nucleotide); (ii) conductance or transport of a 54414 or 53763 substrate across a cellular membrane; (iii) interaction with a second protein (e.g., a second 54414 or 53763 subunit or a non-54414 or 53763 potassium channel subunit); (iv) modulation (e.g., maintenance and/or rectification) of membrane potentials; (v) regulation of target molecule availability or activity; (vi) modulation of intra- or intercellular signaling and/or gene transcription (e.g., either directly or indirectly); (viii) generation of outwardly rectifying currents; (viii) modulation of membrane excitability; (ix) modulation of the release of neurotransmitters; (x) regulation of contractility (e.g., of smooth muscle cells), secretion, and/or synaptic transmission; and/or (xi) modulation of processes which underlie learning and memory.

Preferred activities of 54414 further include at least one of the following activities: (i) interaction with maxi-K potassium channels (i.e., large conductance channels, in particular Slo); (ii) modulation of maxi-K potassium channel activity (e.g., Slo-mediated activities); (iii) generation of intermediate conductance channels; and/or (iv) regulation of contractility (e.g., of smooth muscle cells), secretion, and/or synaptic transmission, in particular, via modulation of Slo.

Preferred activities of 53763 further include at least one of the following activities: (i) interaction with Shaker (Sh) potassium channels and/or channel subunits; (ii) modulation of Shaker (Sh) potassium channel activity (e.g., termination of prolonged membrane depolarization; (iii) modulation of high voltage activating channel activity and/or inactivating channel activity, and the like.

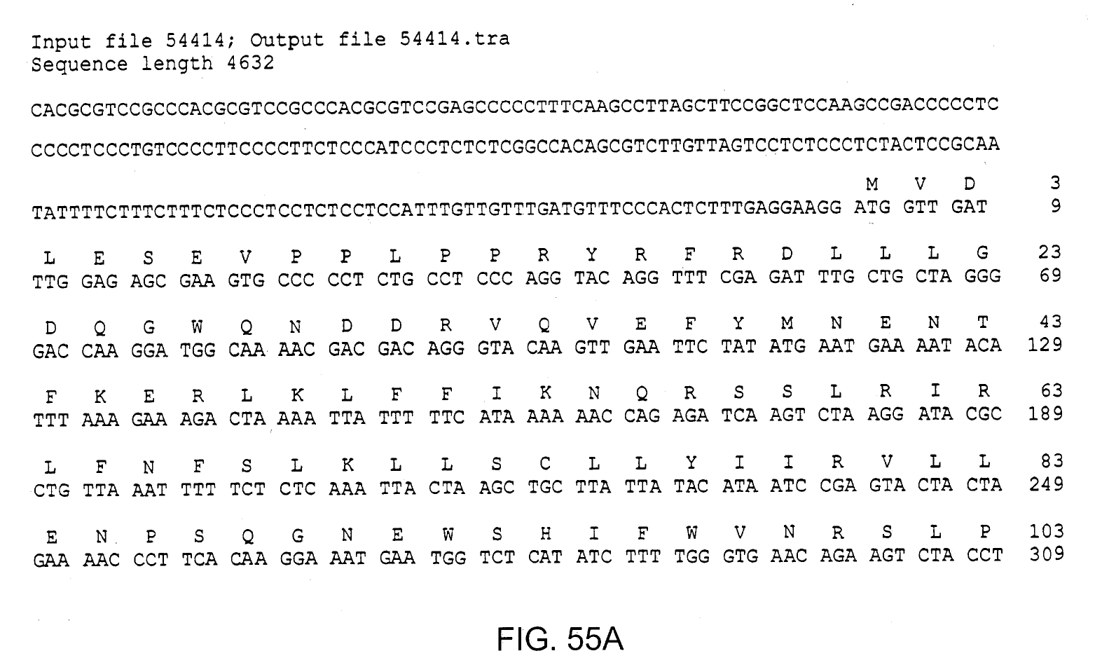

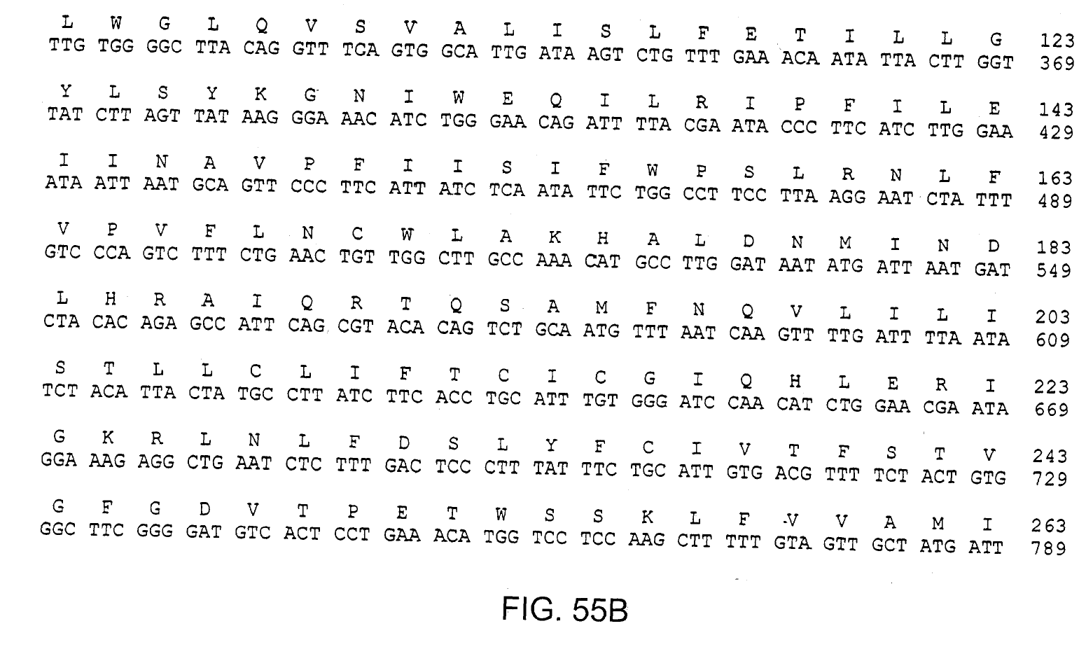

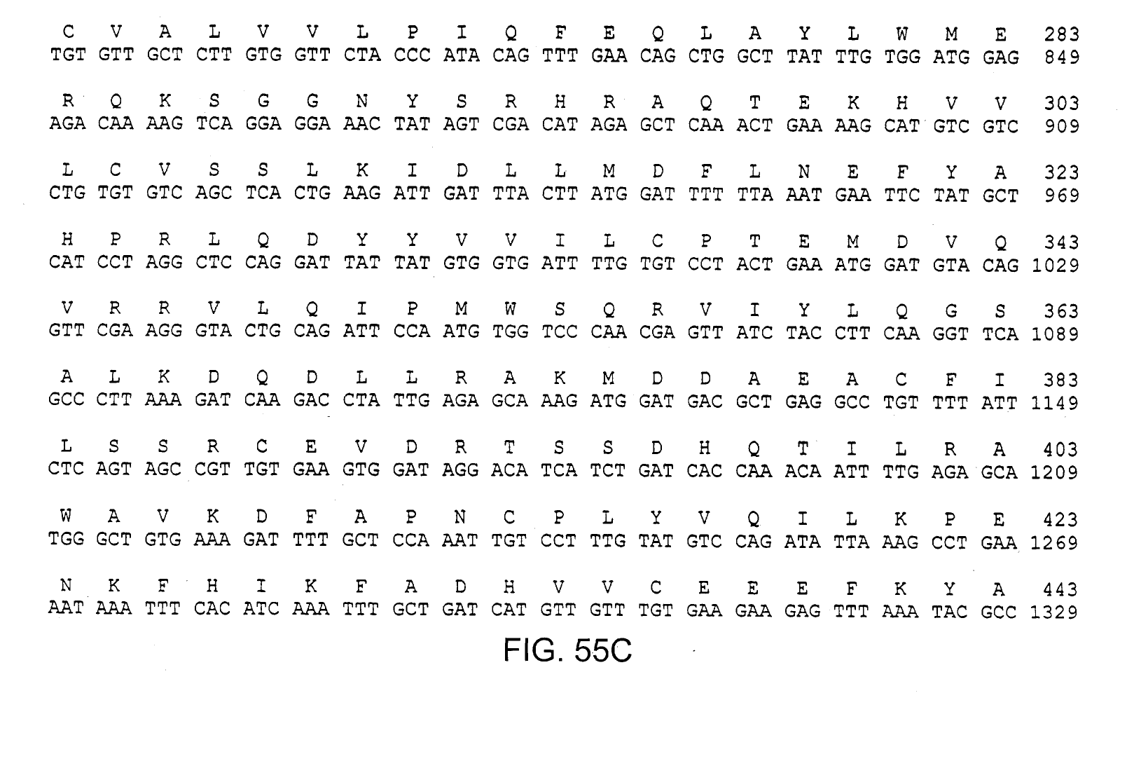

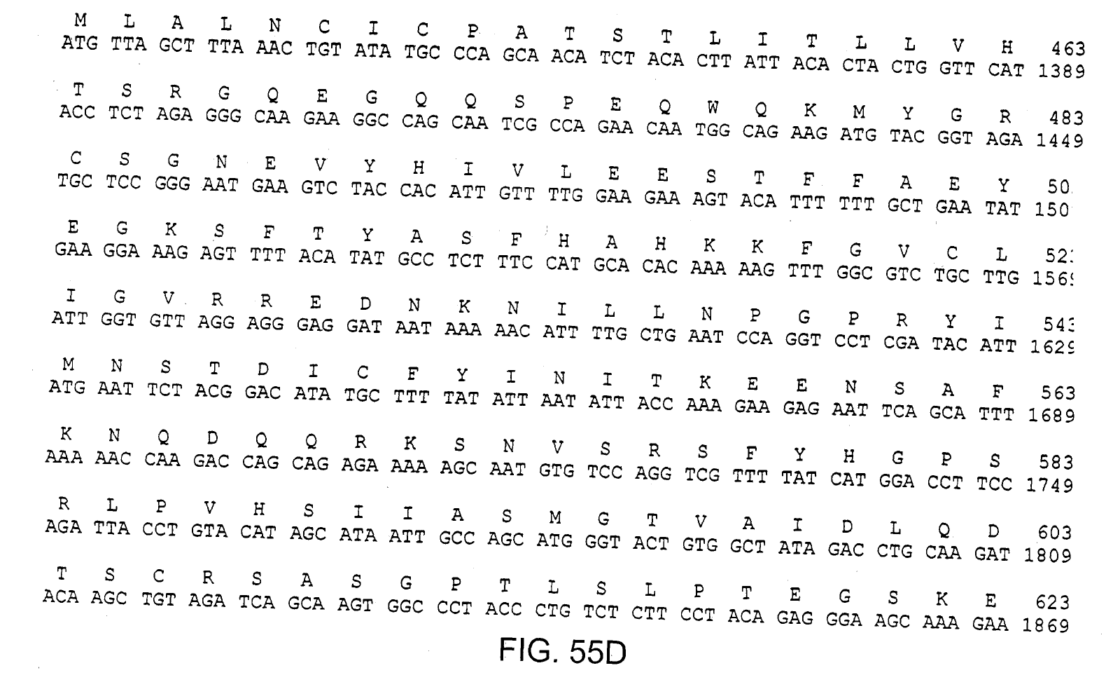

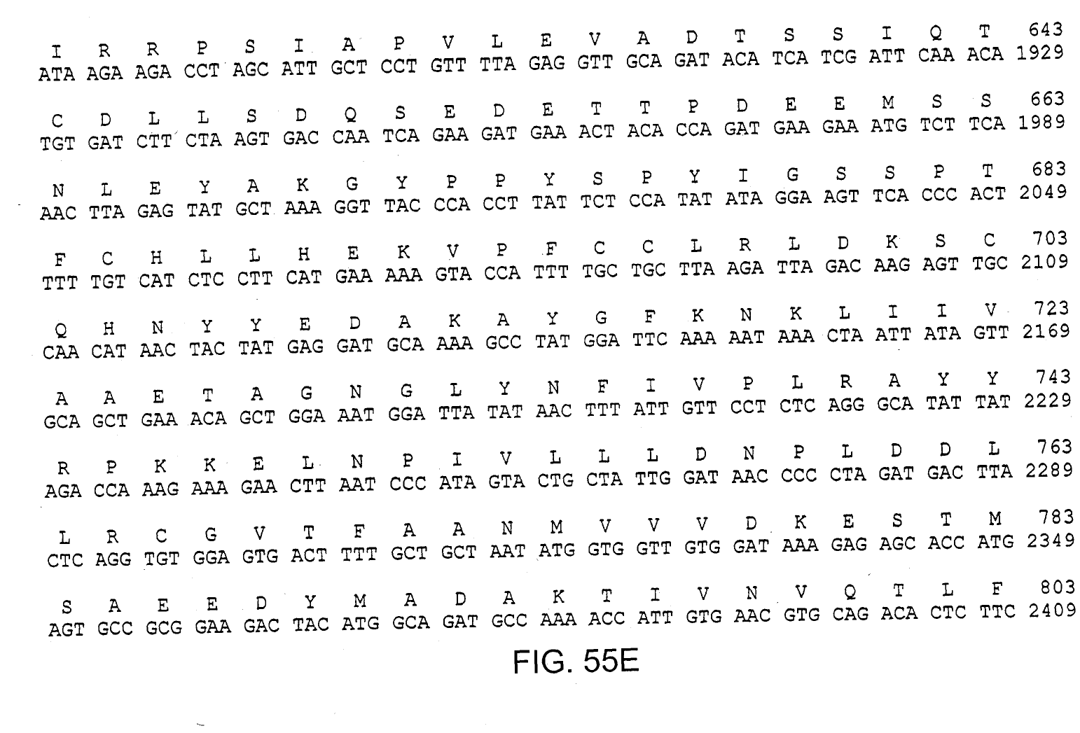

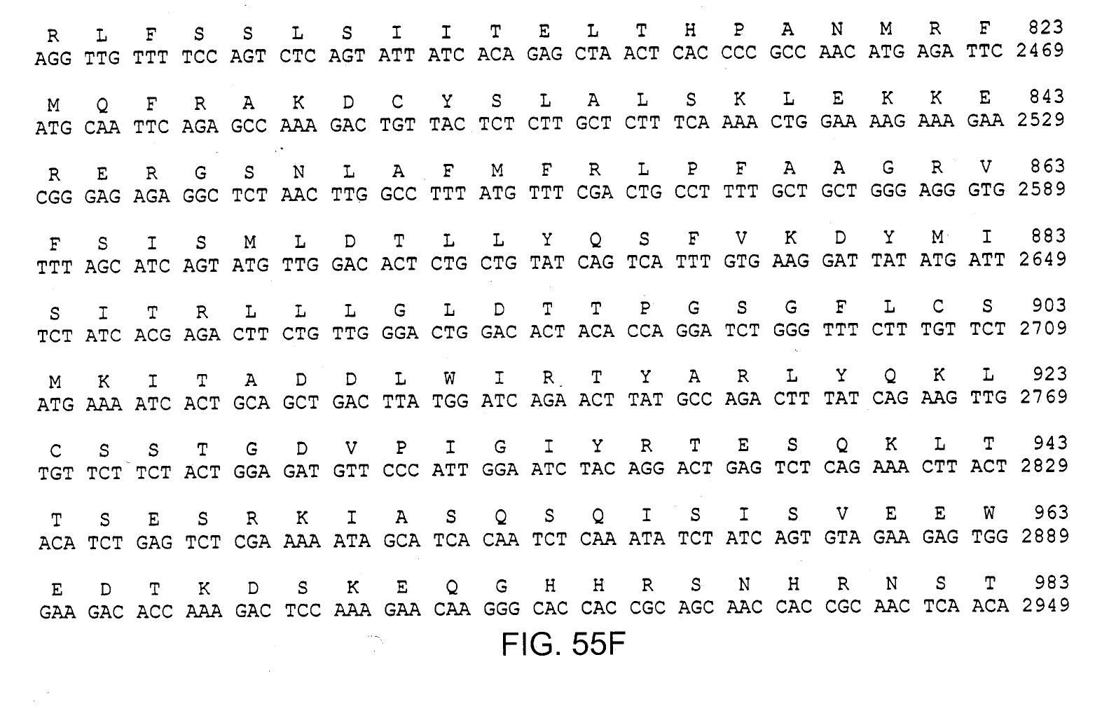

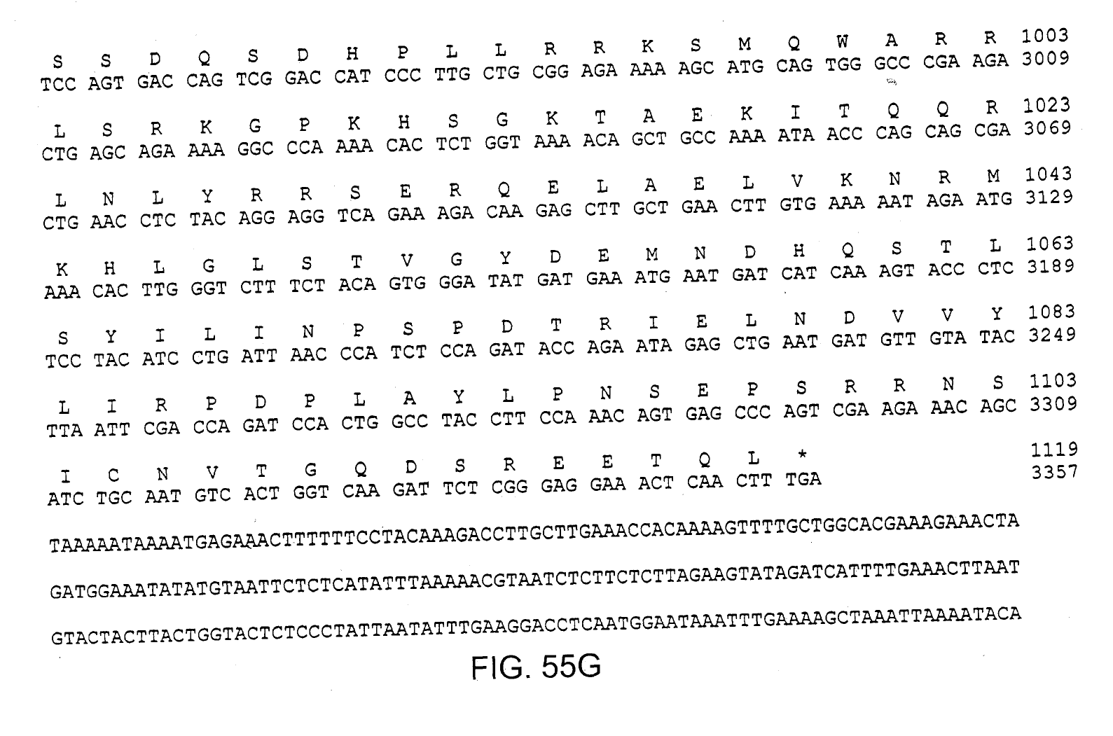

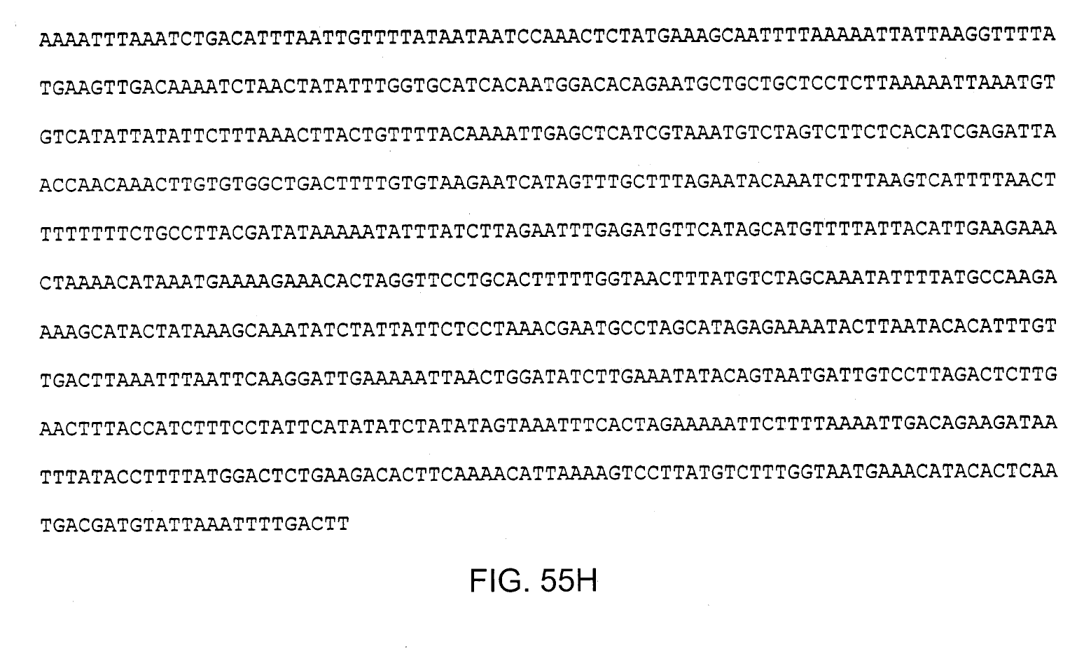

The nucleotide sequence of the isolated human 54414 cDNA and the predicted amino acid sequence encoded by the 54414 cDNA are shown in FIGS. 55A-55H and in SEQ ID NOs:52 and 53, respectively. A plasmid containing the human 54414 cDNA was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit were made merely as a convenience for those of skill in the art and is not an admission that a deposit is required under 35 U.S.C. §112.

The human 54414 gene, which is approximately 4632 nucleotides in length, encodes a protein having a molecular weight of approximately 123 kD and which is approximately 1118 amino acid residues in length.

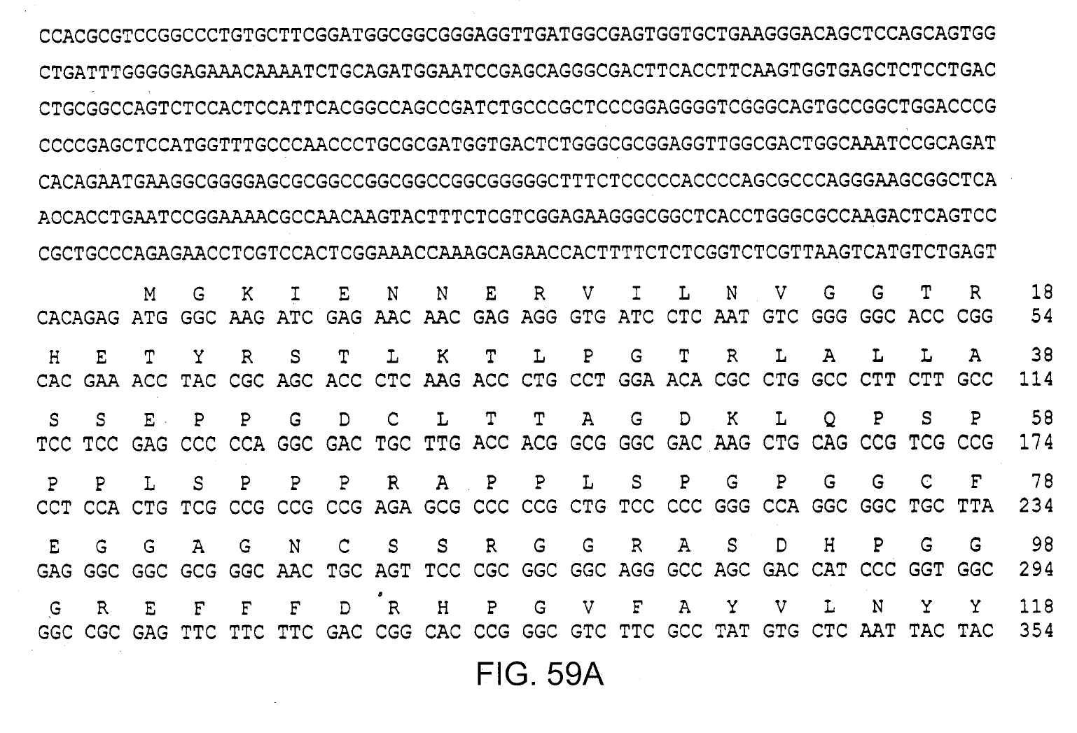

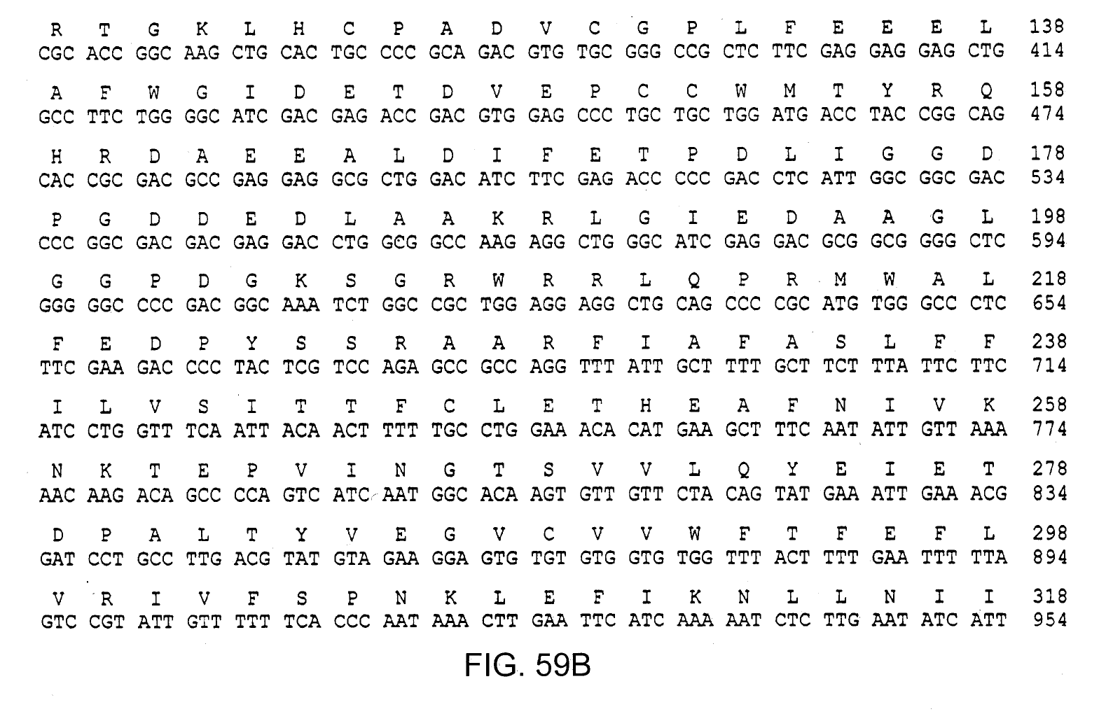

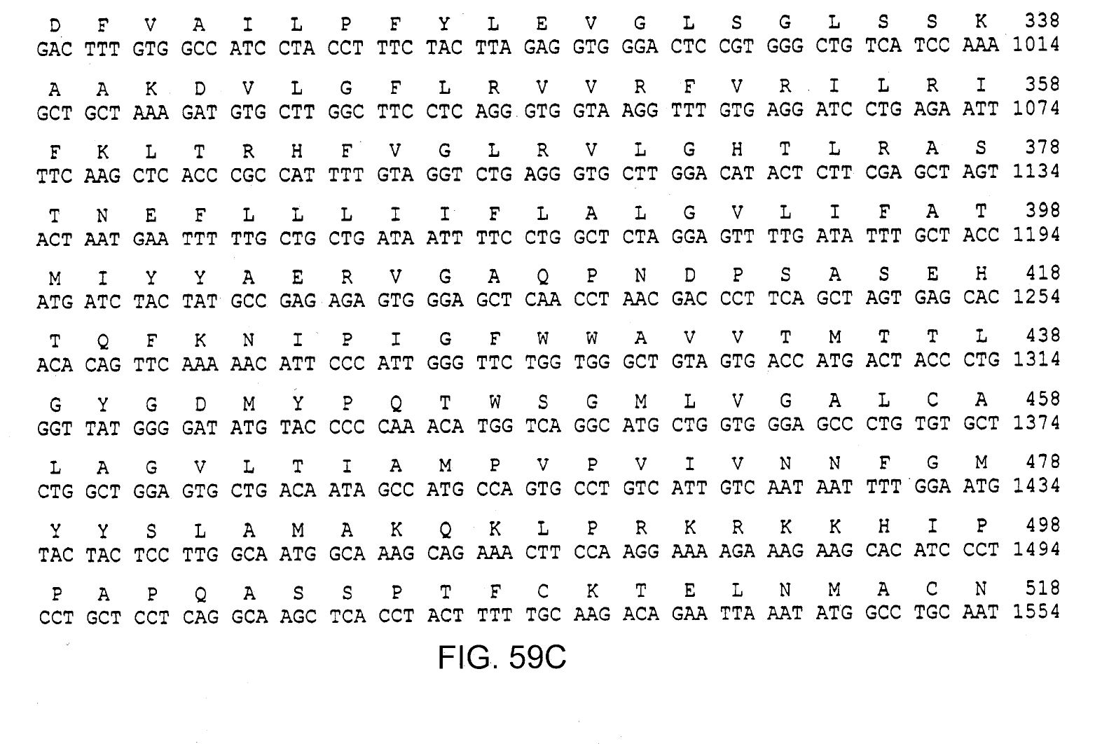

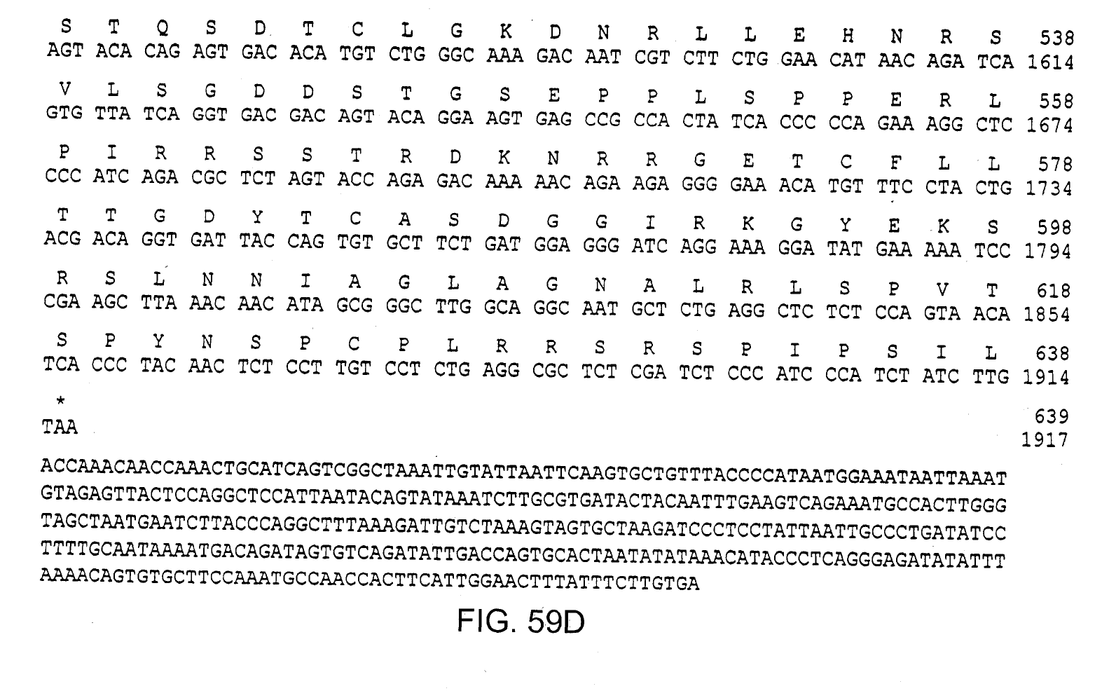

The nucleotide sequence of the isolated human 53763 cDNA and the predicted amino acid sequence encoded by the 53763 cDNA are shown in FIGS. 59A-59D and in SEQ ID NOs:55 and 56, respectively. A plasmid containing the human 53763 cDNA was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit were made merely as a convenience for those of skill in the art and is not an admission that a deposit is required under 35 U.S.C. §112.

The human 53763 gene, which is approximately 2847 nucleotides in length, encodes a protein having a molecular weight of approximately 70.2 kD and which is approximately 638 amino acid residues in length.

67076, 67102, 44181, 67084FL, and 67084alt Molecules of the Invention

The 67076, 67102, 44181, 67084FL, and 67084alt polypeptides comprise at least one “transmembrane domain” and preferably eight, nine, or ten transmembrane domains. A MEMSAT analysis and a structural, hydrophobicity, and antigenicity analysis also resulted in the identification of ten transmembrane domains in the amino acid sequence of human 67076 (SEQ ID NO:59) at about residues 57-77, 84-105, 292-313, 345-365, 863-883, 905-926, 956-977, 989-1009, 1021-1041, and 1060-1087. A MEMSAT analysis and a structural, hydrophobicity, and antigenicity analysis resulted in the identification of ten transmembrane domains in the amino acid sequence of human 67102 (SEQ ID NO:62) at about residues 98-115, 122-140, 322-344, 366-390, 582-601, 752-770, 1145-1166, 1225-1246, 1253-1276, and 1298-1317. A MEMSAT analysis and a structural, hydrophobicity, and antigenicity analysis resulted in the identification of ten transmembrane domains in the amino acid sequence of human 44181 (SEQ ID NO:65) at about residues 56-72, 87-103, 290-311, 343-363, 878-898, 911-931, 961-982, 995-1015, 1027-1047, and 1062-1086. A MEMSAT analysis and a structural, hydrophobicity, and antigenicity analysis resulted in the identification of ten transmembrane domains in the amino acid sequence of human 67084FL (SEQ ID NO:68) at about residues 104-120, 124-144, 331-350, 357-374, 887-903, 912-931, 961-983, 990-1008, 1015-1035, and 1043-1067. A MEMSAT analysis and a structural, hydrophobicity, and antigenicity analysis resulted in the identification of ten transmembrane domains in the amino acid sequence of human 67084alt (SEQ ID NO:71) at about residues 104-120, 124-144, 331-350, 357-379, 887-903, 912-931, 961-983, 990-1008, 1015-1035, and 1054-1078.

The family of 67076, 67102, 44181, 67084FL, or 67084alt proteins of the present invention also comprises at least one “extramembrane domain” in the protein or corresponding nucleic acid molecule. As used herein, an “extramembrane domain” includes a domain having greater than 20 amino acid residues that is found between transmembrane domains, preferably on the cytoplasmic side of the plasma membrane, and does not span or traverse the plasma membrane. An extramembrane domain preferably includes at least one, two, three, four or more motifs or consensus sequences characteristic of P-type ATPases, i.e., includes one, two, three, four, or more “P-type ATPase consensus sequences or motifs”. As used herein, the phrase “P-type ATPase consensus sequences or motifs” includes any consensus sequence or motif known in the art to be characteristic of P-type ATPases, including, but not limited to, the P-type ATPase sequence 1 motif (as defined herein), the P-type ATPase sequence 2 motif (as defined herein), the P-type ATPase sequence 3 motif (as defined herein), and the E1-E2 ATPases phosphorylation site (as defined herein).

In one embodiment, the family of 67076, 67102, 44181, 67084FL, or 67084alt proteins of the present invention comprises at least one “N-terminal” large extramembrane domain in the protein or corresponding nucleic acid molecule. As used herein, an “N-terminal” large extramembrane domain is found in the N-terminal ⅓^{rd} of the protein, preferably between the second and third transmembrane domains of a 67076, 67102, 44181, 67084FL, or 67084alt protein and includes about 60-300, 80-280, 100-260, 120-240, 140-220, 160-200, or preferably, 180, 185, or 186 amino acid residues. In a preferred embodiment, an N-terminal large extramembrane domain includes at least one P-type ATPase sequence 1 motif (as described herein). An N-terminal large extramembrane domain was identified in the amino acid sequence of human 67076 at about residues 106-291 of SEQ ID NO:59. An N-terminal large extramembrane domain was identified in the amino acid sequence of human 67102 at about residues 141-321 of SEQ ID NO:62. An N-terminal large extramembrane domain was identified in the amino acid sequence of human 44181 at about residues 104-289 of SEQ ID NO:65. An N-terminal large extramembrane domain was identified in the amino acid sequence of human 67084FL at about residues 145-330 of SEQ ID NO:68. An N-terminal large extramembrane domain was identified in the amino acid sequence of human 67087alt at about residues 145-330 of SEQ ID NO:71.

The family of 67076, 67102, 44181, 67084FL, or 67084alt proteins of the present invention also comprises at least one “C-terminal” large extramembrane domain in the protein or corresponding nucleic acid molecule. As used herein, a “C-terminal” large extramembrane domain is found in the C-terminal ⅔^{rds} of the protein, preferably between the fourth and fifth transmembrane domains of a 67076, 67102, 44181, 67084FL, or 67084alt protein and includes about 150-1000, 300-900, 370-850, 400-820, 430-790, 460-760, 430-730, 460-700, 430-670, 460-640, 430-610, 490-580, 510-550, or preferably, 190, 506, or 523 amino acid residues. In a preferred embodiment, a C-terminal large extramembrane domain includes at least one or more of the following motifs: a P-type ATPase sequence 2 motif (as described herein), a P-type ATPase sequence 3 motif (as defined herein), and/or an E1-E2 ATPases phosphorylation site (as defined herein). A C-terminal large extramembrane domain was identified in the amino acid sequence of human 67076 at about residues 366-862 of SEQ ID NO:59. A C-terminal large extramembrane domain was identified in the amino acid sequence of human 67102 at about residues 391-581 of SEQ ID NO:62. A C-terminal large extramembrane domain was identified in the amino acid sequence of human 44181 at about residues 364-877 of SEQ ID NO:65. A C-terminal large extramembrane domain was identified in the amino acid sequence of human 67084FL at about residues 380-886 of SEQ ID NO:68. A C-terminal large extramembrane domain was identified in the amino acid sequence of human 67084alt at about residues 380-886 of SEQ ID NO:71.

In another embodiment, a 67076, 67102, 44181, 67084FL, or 67084alt protein or 67076, 67102, 44181, 67084FL, or 67084alt extramembrane domain is characterized by at least one “P-type ATPase sequence 1 motif” in the protein or corresponding nucleic acid sequence. As used herein, a “P-type ATPase sequence 1 motif” is a conserved sequence motif diagnostic for P-type ATPases (Tang, X. et al. (1996) Science 272:1495-1497; Fagan, M. J. and Saier, M. H. (1994) J. Mol. Evol. 38:57). Amino acid residues of the P-type ATPase sequence 1 motif are involved in the coupling of ATP hydrolysis with transport (e.g., transport of phospholipids). The consensus sequence for a P-type ATPase sequence 1 motif is [DNS]-[QENR]-[SA]-[LIVSAN]-[LIV]-[TSN]-G-E-[SN] (SEQ ID NO:82). The use of amino acids in brackets indicates that the amino acid at the indicated position may be any one of the amino acids within the brackets, e.g., [SA] indicates any of one of either S (serine) or A (alanine). In a preferred embodiment, a P-type ATPase sequence 1 motif is contained within an N-terminal large extramembrane domain. In another preferred embodiment, a P-type ATPase sequence 1 motif in the 67076, 67102, 44181, 67084FL, or 67084alt proteins of the present invention has at least 1, 2, 3, or preferably 4 amino acid resides which match the consensus sequence for a P-type ATPase sequence 1 motif. A P-type ATPase sequence 1 motif was identified in the amino acid sequence of human 67076 at about residues 173-181 of SEQ ID NO:59. A P-type ATPase sequence 1 motif was identified in the amino acid sequence of human 67102 at about residues 208-216 of SEQ ID NO:62. A P-type ATPase sequence 1 motif was identified in the amino acid sequence of human 44181 at about residues 173-181 of SEQ ID NO:65. A P-type ATPase sequence 1 motif was identified in the amino acid sequence of human 67084FL at about residues 213-221 of SEQ ID NO:68. A P-type ATPase sequence 1 motif was identified in the amino acid sequence of human 67084alt at about residues 213-221 of SEQ ID NO:71.

In another embodiment, a 67076, 67102, 44181, 67084FL, or 67084alt protein or 67076, 67102, 44181, 67084FL, or 67084alt extramembrane domain is characterized by at least one “P-type ATPase sequence 2 motif” in the protein or corresponding nucleic acid sequence. As used herein, a “P-type ATPase sequence 2 motif” is a conserved sequence motif diagnostic for P-type ATPases (Tang, X. et al. (1996) Science 272:1495-1497; Fagan, M. J. and Saier, M. H. (1994) J. Mol. Evol. 38:57). Preferably, a P-type ATPase sequence 2 motif overlaps with and/or includes an E1-E2 ATPases phosphorylation site (as defined herein). The consensus sequence for a P-type ATPase sequence 2 motif is [LIV]-[CAML]-[STFL]-D-K-T-G-T-[LI]-T (SEQ ID NO:83). The use of amino acids in brackets indicates that the amino acid at the indicated position may be any one of the amino acids within the brackets, e.g., [LI] indicates any of one of either L (leucine) or I (isoleucine). In a preferred embodiment, a P-type ATPase sequence 2 motif is contained within a C-terminal large extramembrane domain. In another preferred embodiment, a P-type ATPase sequence 2 motif in the 67076, 67102, 44181, 67084FL, or 67084alt proteins of the present invention has at least 1, 2, 3, 4, 5, 6, 7, 8, or more preferably 9 amino acid resides which match the consensus sequence for a P-type ATPase sequence 2 motif. A P-type ATPase sequence 2 motif was identified in the amino acid sequence of human 67076 at about residues 406-415 of SEQ ID NO:59. A P-type ATPase sequence 2 motif was identified in the amino acid sequence of human 67102 at about residues 435-444 of SEQ ID NO:62. A P-type ATPase sequence 2 motif was identified in the amino acid sequence of human 44181 at about residues 404-413 of SEQ ID NO:65. A P-type ATPase sequence 2 motif was identified in the amino acid sequence of human 67084FL at about residues 413-422 of SEQ ID NO:68. A P-type ATPase sequence 2 motif was identified in the amino acid sequence of human 67084alt at about residues 413-422 of SEQ ID NO:71.

In yet another embodiment, a 67076, 67102, 44181, 67084FL, or 67084alt protein or 67076, 67102, 44181, 67084FL, or 67084alt extramembrane domain is characterized by at least one “P-type ATPase sequence 3 motif” in the protein or corresponding nucleic acid sequence. As used herein, a “P-type ATPase sequence 3 motif” is a conserved sequence motif diagnostic for P-type ATPases (Tang, X. et al. (1996) Science 272:1495-1497; Fagan, M. J. and Saier, M. H. (1994) J. Mol. Evol. 38:57). Amino acid residues of the P-type ATPase sequence 3 motif are involved in ATP binding. The consensus sequence for a P-type ATPase sequence 3 motif is [TIV]-G-D-G-X-N-D-[ASG]-P-[ASV]-L (SEQ ID NO:84). X indicates that the amino acid at the indicated position may be any amino acid (i.e., is not conserved). The use of amino acids in brackets indicates that the amino acid at the indicated position may be any one of the amino acids within the brackets, e.g., [TIV] indicates any of one of either T (threonine), I (isoleucine), or V (valine). In a preferred embodiment, a P-type ATPase sequence 3 motif is contained within a C-terminal large extramembrane domain. In another preferred embodiment, a P-type ATPase sequence 3 motif in the 67076, 67102, 44181, 67084FL, or 67084alt proteins of the present invention has at least 1, 2, 3, 4, 5, 6, or more preferably 7 amino acid resides (including the amino acid at the position indicated by “X”) which match the consensus sequence for a P-type ATPase sequence 3 motif. A P-type ATPase sequence 3 motif was identified in the amino acid sequence of human 67076 at about residues 813-824 of SEQ ID NO:59. A P-type ATPase sequence 3 motif was identified in the amino acid sequence of human 67102 at about residues 1054-1064 of SEQ ID NO:62. A P-type ATPase sequence 3 motif was identified in the amino acid sequence of human 44181 at about residues 819-829 of SEQ ID NO:65. A P-type ATPase sequence 3 motif was identified in the amino acid sequence of human 67084FL at about residues 820-830 of SEQ ID NO:68. A P-type ATPase sequence 3 motif was identified in the amino acid sequence of human 67084alt at about residues 820-830 of SEQ ID NO:71.

In another embodiment, a 67076, 67102, 44181, 67084FL, or 67084alt protein of the present invention is identified based on the presence of an “E1-E2 ATPases phosphorylation site” (alternatively referred to simply as a “phosphorylation site”) in the protein or corresponding nucleic acid molecule. An E1-E2 ATPases phosphorylation site functions in accepting a phosphate moiety and has the amino acid sequence DKTGT (amino acid residues 4-8 of SEQ ID NO:83), and can be included within the E1-E2 ATPase phosphorylation site consensus sequence: D-K-T-G-T-[LIVM]-[TI] (SEQ ID NO:85), wherein D is phosphorylated. The use of amino acids in brackets indicates that the amino acid at the indicated position may be any one of the amino acids within the brackets, e.g., [TI] indicates any of one of either T (threonine) or I (isoleucine). The E1-E2 ATPases phosphorylation site consensus sequence has been assigned ProSite Accession Number PS00154. To identify the presence of an E1-E2 ATPases phosphorylation site consensus sequence in a 67076, 67102, 44181, 67084FL, or 67084alt protein, and to make the determination that a protein of interest has a particular profile, the amino acid sequence of the protein may be searched against a database of known protein motifs (e.g., the ProSite database) using the default parameters (available at the Prosite website). A search was performed against the ProSite database resulting in the identification of an E1-E2 ATPases phosphorylation site consensus sequence in the amino acid sequence of human 67076 (SEQ ID NO:59) at about residues 409-415. A search was performed against the ProSite database resulting in the identification of an E1-E2 ATPases phosphorylation site consensus sequence in the amino acid sequence of human 67102 (SEQ ID NO:62) at about residues 438-444. A search was performed against the ProSite database resulting in the identification of an E1-E2 ATPases phosphorylation site consensus sequence in the amino acid sequence of human 44181 (SEQ ID NO:65) at about residues 407-413. A search was performed against the ProSite database resulting in the identification of an E1-E2 ATPases phosphorylation site consensus sequence in the amino acid sequence of human 67084FL (SEQ ID NO:68) at about residues 416-422. A search was performed against the ProSite database resulting in the identification of an E1-E2 ATPases phosphorylation site consensus sequence in the amino acid sequence of human 67084alt (SEQ ID NO:71) at about residues 416-422.

Preferably an E1-E2 ATPases phosphorylation site has a “phosphorylation site activity,” for example, the ability to be phosphorylated; to be dephosphorylated; to regulate the E1-E2 conformational change of the phospholipid transporter in which it is contained; to regulate transport of phospholipids (e.g., aminophospholipids such as phosphatidylserine and phosphatidylethanolamine, choline phospholipids such as phosphatidylcholine and sphingomyelin, and bile acids) across a cellular membrane by the 67076, 67102, 44181, 67084FL, or 67084alt protein in which it is contained; and/or to regulate the activity (as defined herein) of the 67076, 67102, 44181, 67084FL, or 67084alt protein in which it is contained. Accordingly, identifying the presence of an “E1-E2 ATPases phosphorylation site” can include isolating a fragment of a 67076, 67102, 44181, 67084FL, or 67084alt molecule (e.g., a 67076, 67102, 44181, 67084FL, or 67084alt polypeptide) and assaying for the ability of the fragment to exhibit one of the aforementioned phosphorylation site activities.

In another embodiment, a 67076, 67102, 44181, 67084FL, or 67084alt protein of the present invention may also be identified based on its ability to adopt an E1 conformation or an E2 conformation. As used herein, an “E1 conformation” of a 67076, 67102, 44181, 67084FL, or 67084alt protein includes a 3-dimensional conformation of a 67076, 67102, 44181, 67084FL, or 67084alt protein which does not exhibit 67076, 67102, 44181, 67084FL, or 67084alt activity (e.g., the ability to transport phospholipids), as defined herein. An E1 conformation of a 67076, 67102, 44181, 67084FL, or 67084alt protein usually occurs when the 67076, 67102, 44181, 67084FL, or 67084alt protein is unphosphorylated. As used herein, an “E2 conformation” of a 67076, 67102, 44181, 67084FL, or 67084alt protein includes a 3-dimensional conformation of a 67076, 67102, 44181, 67084FL, or 67084alt protein which exhibits 67076, 67102, 44181, 67084FL, or 67084alt activity (e.g., the ability to transport phospholipids), as defined herein. An E2 conformation of a 67076, 67102, 44181, 67084FL, or 67084alt protein usually occurs when the 67076, 67102, 44181, 67084FL, or 67084alt protein is phosphorylated.

In still another embodiment, a 67076, 67102, 44181, 67084FL, or 67084alt protein of the present invention is identified based on the presence of “phospholipid transporter specific” amino acid residues. As used herein, “phospholipid transporter specific” amino acid residues are amino acid residues specific to the class of phospholipid transporting P-type ATPases (as defined in Tang, X. et al. (1996) Science 272:1495-1497). Phospholipid transporter specific amino acid residues are not found in those P-type ATPases which transport molecules which are not phospholipids (e.g., cations). For example, phospholipid transporter specific amino acid residues are found at the first, second, and fifth positions of the P-type ATPase sequence 1 motif. In phospholipid transporting P-type ATPases, the first position of the P-type ATPase sequence 1 motif is preferably E (glutamic acid), the second position is preferably T (threonine), and the fifth position is preferably L (leucine). A phospholipid transporter specific amino acid residue is further found at the second position of the P-type ATPase sequence 2 motif. In phospholipid transporting P-type ATPases, the second position of the P-type ATPase sequence 2 motif is preferably F (phenylalanine). Phospholipid transporter specific amino acid residues are still further found at the first, tenth, and eleventh positions of the P-type ATPase sequence 3 motif. In phospholipid transporting P-type ATPases, the first position of the P-type ATPase sequence 3 motif is preferably I (isoleucine), the tenth position is preferably M (methionine), and the eleventh position is preferably I (isoleucine).

Phospholipid transporter specific amino acid residues were identified in the amino acid sequence of human 67076 (SEQ ID NO:59) at about residues 174 and 177 (within the P-type ATPase sequence 1 motif), at about residue 407 (within the P-type ATPase sequence 2 motif), and at about residues 813, 823, and 824 (within the P-type ATPase sequence 3 motif).

Phospholipid transporter specific amino acid residues were identified in the amino acid sequence of human 67102 (SEQ ID NO:62) at about residues 208, 209, and 212 (within the P-type ATPase sequence 1 motif), at about residue 436 (within the P-type ATPase sequence 2 motif), and at about residues 1054, 1063, and 1064 (within the P-type ATPase sequence 3 motif).

Phospholipid transporter specific amino acid residues were identified in the amino acid sequence of human 44181 (SEQ ID NO:65) at about residues 174 and 177 (within the P-type ATPase sequence 1 motif), at about residue 405 (within the P-type ATPase sequence 2 motif), and at about residues 819, 828, and 829 (within the P-type ATPase sequence 3 motif).

Phospholipid transporter specific amino acid residues were identified in the amino acid sequence of human 67084FL (SEQ ID NO:68) at about residues 214 and 217 (within the P-type ATPase sequence 1 motif) and at about residues 820, 829, and 830 (within the P-type ATPase sequence 3 motif).

Phospholipid transporter specific amino acid residues were identified in the amino acid sequence of human 67084alt (SEQ ID NO:71) at about residues 214 and 217 (within the P-type ATPase sequence 1 motif), and at about residues 820, 829, and 830 (within the P-type ATPase sequence 3 motif).

Isolated polypeptides of the present invention, preferably 67076, 67102, 44181, 67084FL, or 67084alt polypeptides, have an amino acid sequence sufficiently identical to the amino acid sequence of SEQ ID NO:59, 62, 65, 68, or 71 or are encoded by a nucleotide sequence sufficiently identical to SEQ ID NO:58, 60, 61, 63, 64, 66, 67, 69, 70, or 72. For example, amino acid or nucleotide sequences which share common structural domains having at least 50%, 55%, 60%, 65%, 70%, 75%, 80%, 85%, 85%, 90%, 95%, 96%, 97%, 98%, 99% or more homology or identity across the amino acid sequences of the domains and contain at least one and preferably two structural domains or motifs, are defined herein as sufficiently identical. Furthermore, amino acid or nucleotide sequences which share at least 50%, 55%, 60%, 65%, 70%, 75%, 80%, 85%, 85%, 90%, 95%, 96%, 97%, 98%, 99% or more homology or identity and share a common functional activity are defined herein as sufficiently identical.

In a preferred embodiment, a 67076, 67102, 44181, 67084FL, or 67084alt protein includes at least one or more of the following domains, sites, or motifs: a transmembrane domain, an N-terminal large extramembrane domain, a C-terminal large extramembrane domain, an E1-E2 ATPases phosphorylation site, a P-type ATPase sequence 1 motif, a P-type ATPase sequence 2 motif, a P-type ATPase sequence 3 motif, and/or one or more phospholipid transporter specific amino acid resides, and has an amino acid sequence at least about 50%, 55%, 60%, 65%, 70%, 75%, 80%, 85%, 85%, 90%, 95%, 96%, 97%, 98%, 99% or more homologous or identical to the amino acid sequence of SEQ ID NO:59, 62, 65, 68, or 71, or the amino acid sequence encoded by the DNA insert of the plasmid deposited with ATCC as Accession Number ______, ______, ______, ______, or ______. In yet another preferred embodiment, a 67076, 67102, 44181, 67084FL, or 67084alt protein includes at least one or more of the following domains, sites, or motifs: a transmembrane domain, an N-terminal large extramembrane domain, a C-terminal large extramembrane domain, an E1-E2 ATPases phosphorylation site, a P-type ATPase sequence 1 motif, a P-type ATPase sequence 2 motif, a P-type ATPase sequence 3 motif, and/or one or more phospholipid transporter specific amino acid resides, and is encoded by a nucleic acid molecule having a nucleotide sequence which hybridizes under stringent hybridization conditions to a complement of a nucleic acid molecule comprising the nucleotide sequence of SEQ ID NO:58, 60, 61, 63, 64, 66, 67, 69, 70, or 72. In another preferred embodiment, a 67076, 67102, 44181, 67084FL, or 67084alt protein includes at least one or more of the following domains, sites, or motifs: a transmembrane domain, an N-terminal large extramembrane domain, a C-terminal large extramembrane domain, an E1-E2 ATPases phosphorylation site, a P-type ATPase sequence 1 motif, a P-type ATPase sequence 2 motif, a P-type ATPase sequence 3 motif, and/or one or more phospholipid transporter specific amino acid resides, and has a 67076, 67102, 44181, 67084FL, or 67084alt activity.

As used interchangeably herein, a “phospholipid transporter activity” or a “67076, 67102, 44181, 67084FL, or 67084alt activity” includes an activity exerted or mediated by a 67076, 67102, 44181, 67084FL, or 67084alt protein, polypeptide or nucleic acid molecule on a 67076, 67102, 44181, 67084FL, or 67084alt responsive cell or on a 67076, 67102, 44181, 67084FL, or 67084alt substrate, as determined in vivo or in vitro, according to standard techniques. In one embodiment, a phospholipid transporter activity is a direct activity, such as an association with a 67076, 67102, 44181, 67084FL, or 67084alt target molecule. As used herein, a “target molecule” or “binding partner” is a molecule with which a 67076, 67102, 44181, 67084FL, or 67084alt protein binds or interacts in nature, such that 67076, 67102, 44181, 67084FL, or 67084alt-mediated function is achieved. In an exemplary embodiment, a 67076, 67102, 44181, 67084FL, or 67084alt target molecule is a 67076, 67102, 44181, 67084FL, or 67084alt substrate (e.g., a phospholipid, ATP, or a non-67076, 67102, 44181, 67084FL, or 67084alt protein). A phospholipid transporter activity can also be an indirect activity, such as a cellular signaling activity mediated by interaction of the 67076, 67102, 44181, 67084FL, or 67084alt protein with a 67076, 67102, 44181, 67084FL, or 67084alt substrate.

In a preferred embodiment, a phospholipid transporter activity is at least one of the following activities: (i) interaction with a 67076, 67102, 44181, 67084FL, or 67084alt substrate or target molecule (e.g., a phospholipid, ATP, or a non-67076, 67102, 44181, 67084FL, or 67084alt protein); (ii) transport of a 67076, 67102, 44181, 67084FL, or 67084alt substrate or target molecule (e.g., an aminophospholipid such as phosphatidylserine or phosphatidylethanolamine) from one side of a cellular membrane to the other; (iii) the ability to be phosphorylated or dephosphorylated; (iv) adoption of an E1 conformation or an E2 conformation; (v) conversion of a 67076, 67102, 44181, 67084FL, or 67084alt substrate or target molecule to a product (e.g., hydrolysis of ATP); (vi) interaction with a second non-67076, 67102, 44181, 67084FL, or 67084alt protein; (vii) modulation of substrate or target molecule location (e.g., modulation of phospholipid location within a cell and/or location with respect to a cellular membrane); (viii) maintenance of aminophospholipid gradients; (ix) modulation of intra- or intercellular signaling and/or gene transcription (e.g., either directly or indirectly); and/or (x) modulation of cellular proliferation, growth, differentiation, apoptosis, absorption, or secretion.

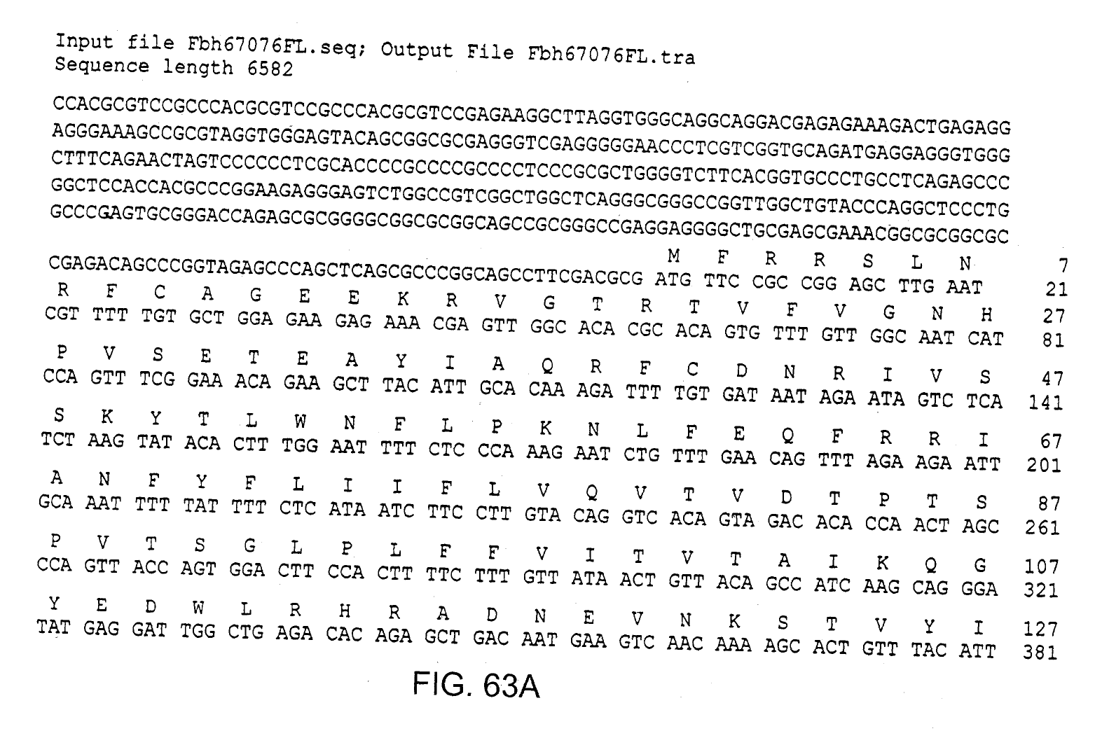

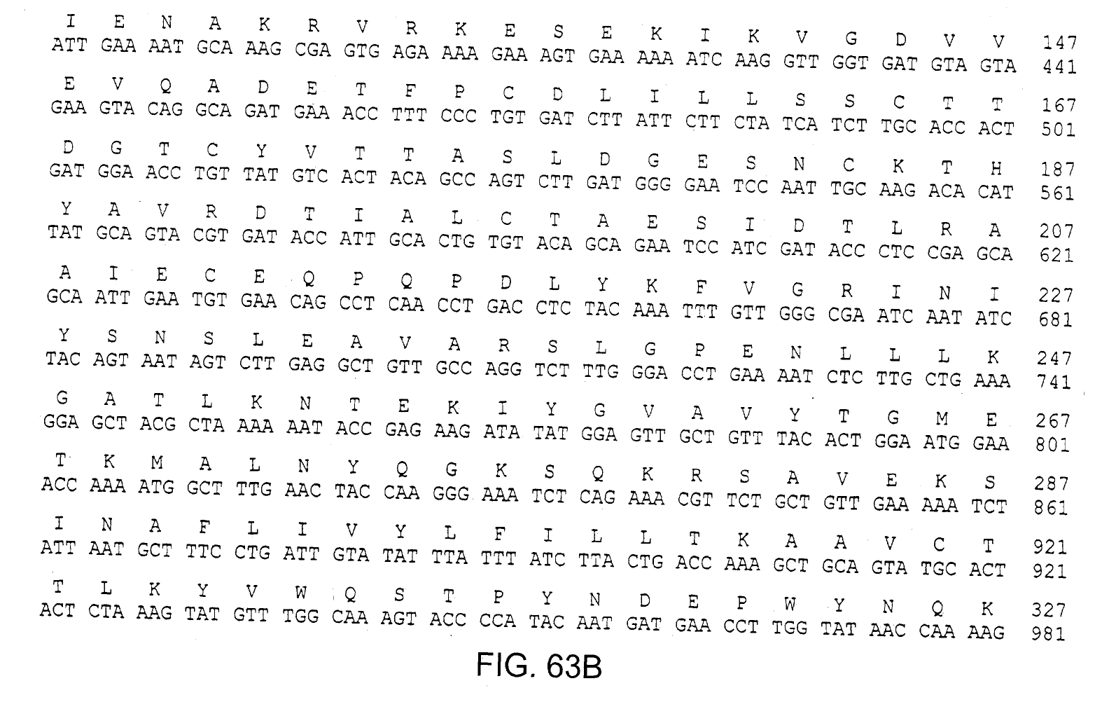

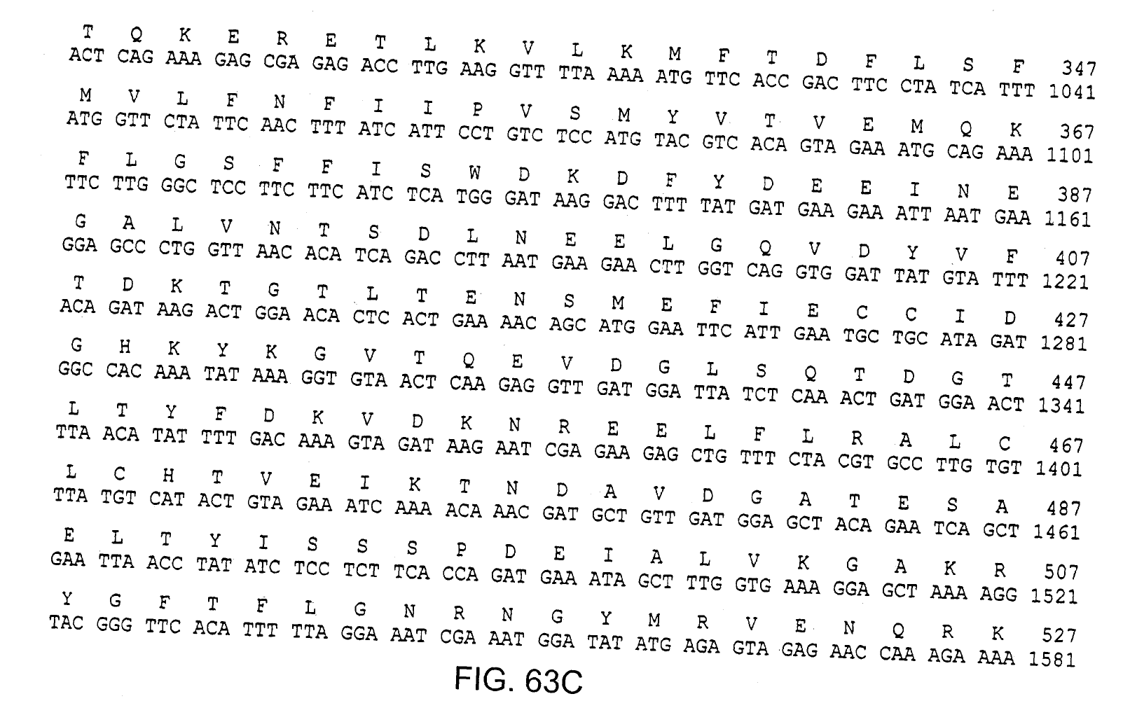

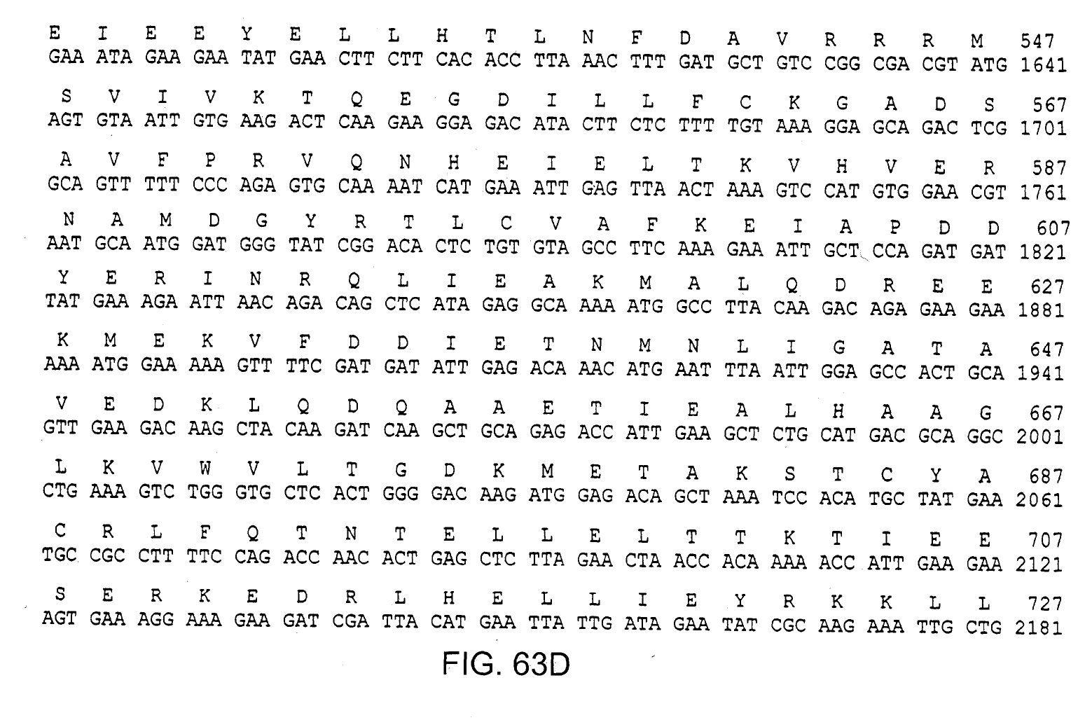

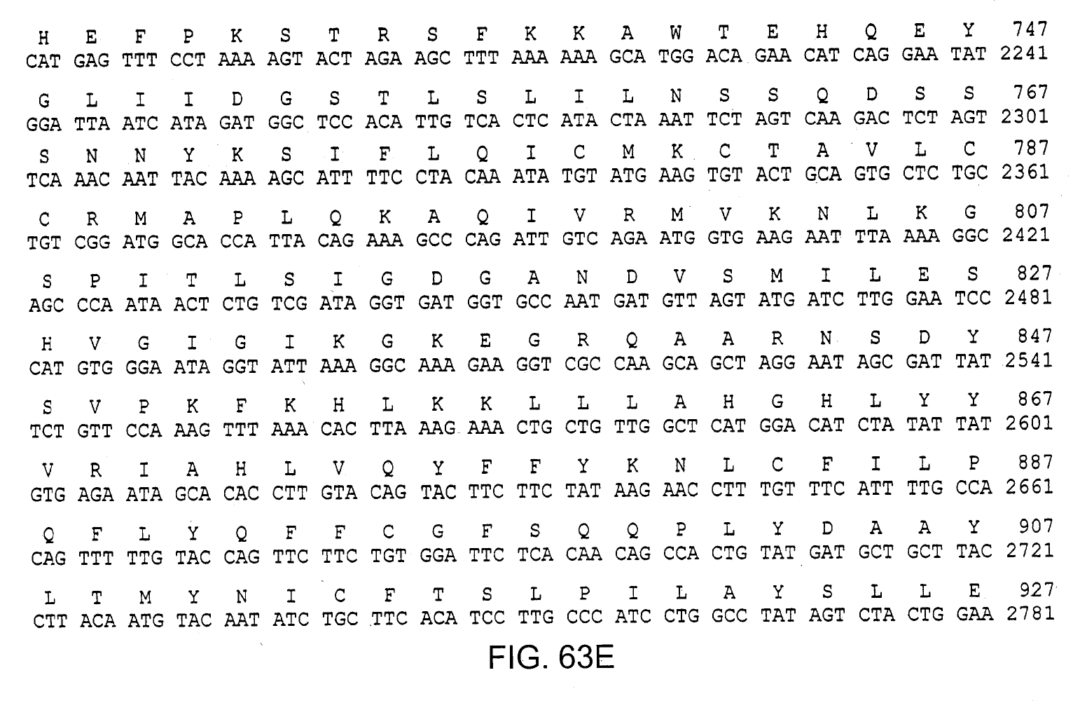

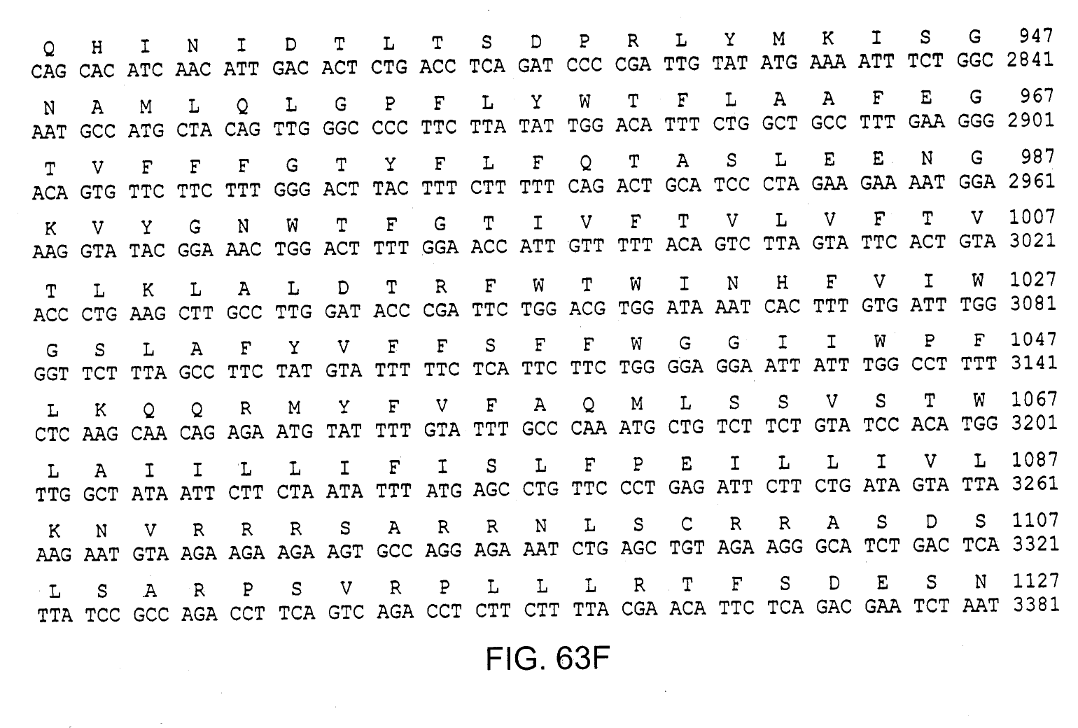

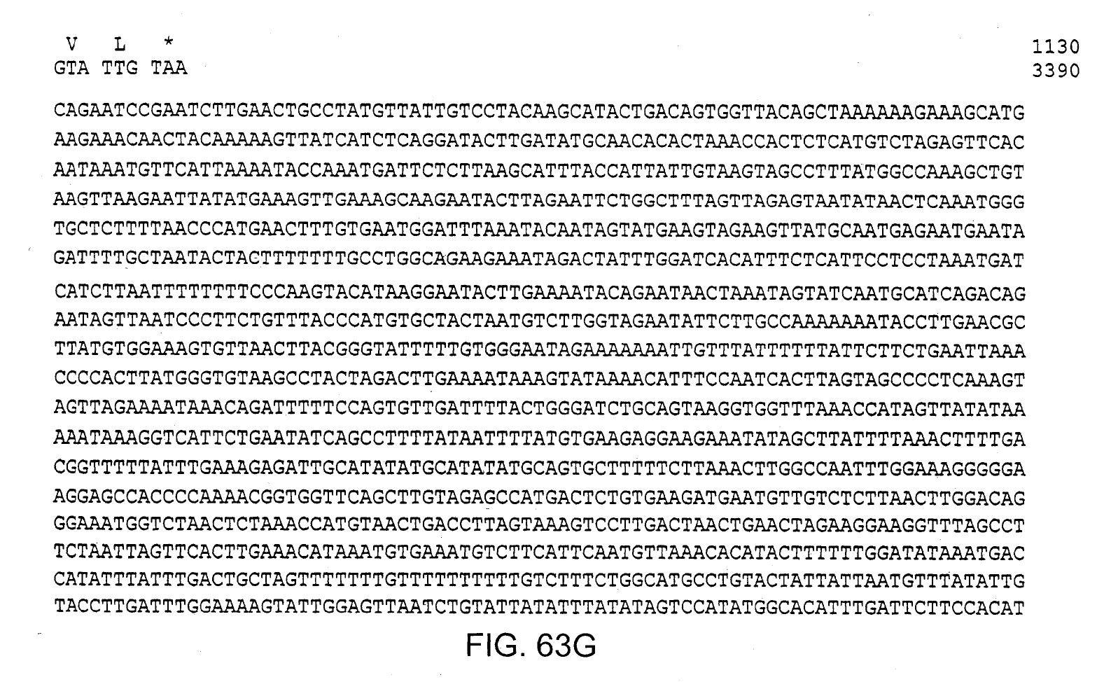

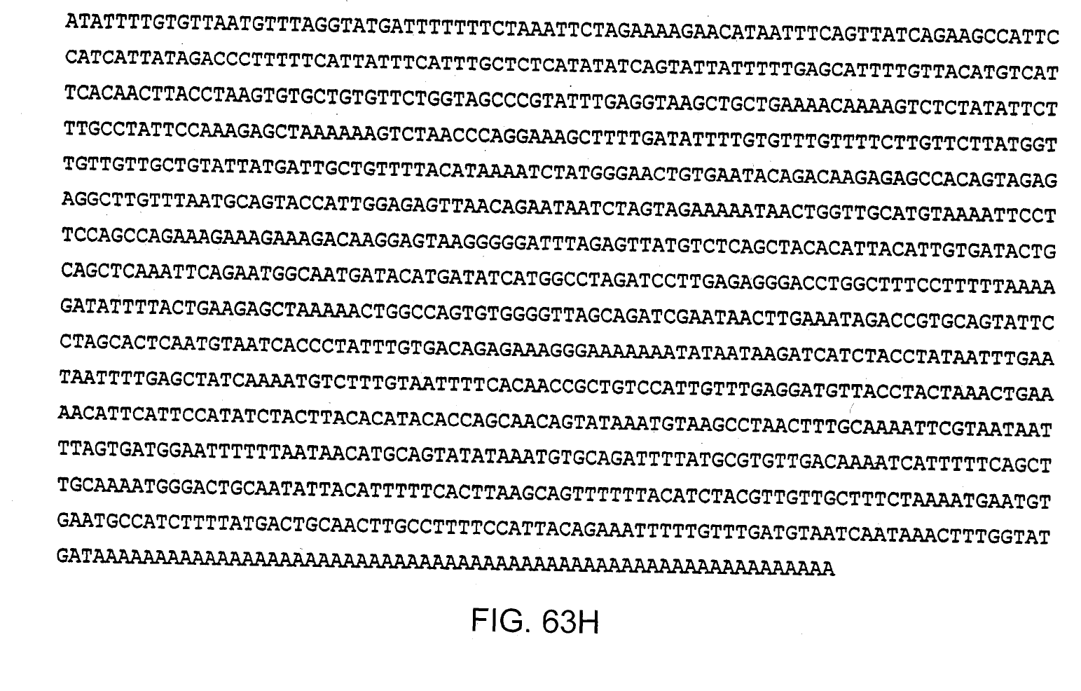

The nucleotide sequence of the isolated human 67076, 67102, 44181, 67084FL, or 67084alt cDNA and the predicted amino acid sequence of the human 67076, 67102, 44181, 67084FL, or 67084alt polypeptides are shown in FIGS. 63A-63H, 67A-67I, 71A-71J, 75A-75G, and 79A-79G, and in SEQ ID NOs:58 and 59, SEQ ID NOs:61 and 62, SEQ ID NOs:64 and 65, SEQ ID NOs:67 and 68, and SEQ ID NOs:70 and 71, respectively. Plasmids containing the nucleotide sequence encoding human 67076, human 67102, human 44181, human 67084FL, and/or human 67084alt were deposited with the American Type Culture Collection (ATCC), 10801 University Boulevard, Manassas, Va. 20110-2209, on ______, ______, ______, ______, and ______, respectively, and assigned Accession Numbers ______, ______, ______, ______, and ______, respectively. These deposits will be maintained under the terms of the Budapest Treaty on the International Recognition of the Deposit of Microorganisms for the Purposes of Patent Procedure. These deposit were made merely as a convenience for those of skill in the art and are not admissions that a deposit is required under 35 U.S.C. §112.

The human 67076 gene, which is approximately 6582 nucleotides in length, encodes a polypeptide which is approximately 1129 amino acid residues in length. The human 67102 gene, which is approximately 6074 nucleotides in length, encodes a polypeptide which is approximately 1426 amino acid residues in length. The human 44181 gene, which is approximately 7221 nucleotides in length, encodes a polypeptide which is approximately 1177 amino acid residues in length. The human 67084FL gene, which is approximately 4198 nucleotides in length, encodes a polypeptide which is approximately 1084 amino acid residues in length. The human 67084alt gene, which is approximately 4231 nucleotides in length, encodes a polypeptide which is approximately 1095 amino acid residues in length.

Various aspects of the invention are described in further detail in the following subsections:

I. Isolated Nucleic Acid Molecules

One aspect of the invention pertains to isolated nucleic acid molecules that encode 8099, 46455, 54414, 53763, 67076, 67102, 44181, 67084FL, or 67084alt polypeptides or biologically active portions thereof, as well as nucleic acid fragments sufficient for use as hybridization probes to identify 8099, 46455, 54414, 53763, 67076, 67102, 44181, 67084FL, or 67084alt-encoding nucleic acid molecules (e.g., 8099, 46455, 54414, 53763, 67076, 67102, 44181, 67084FL, or 67084alt mRNA) and fragments for use as PCR primers for the amplification or mutation of 8099, 46455, 54414, 53763, 67076, 67102, 44181, 67084FL, or 67084alt nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated 8099, 46455, 54414, 53763, 67076, 67102, 44181, 67084FL, or 67084alt nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______ or a portion thereof, can be isolated using standard molecular biology techniques and the sequence information provided herein. Using all or a portion of the nucleic acid sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, as a hybridization probe, 8099, 46455, 54414, 53763, 67076, 67102, 44181, 67084FL, or 67084alt nucleic acid molecules can be isolated using standard hybridization and cloning techniques (e.g., as described in Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989).

Moreover, a nucleic acid molecule encompassing all or a portion of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______ can be isolated by the polymerase chain reaction (PCR) using synthetic oligonucleotide primers designed based upon the sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to 8099, 46455, 54414, 53763, 67076, 67102, 44181, 67084FL, or 67084alt nucleotide sequences can be prepared by standard synthetic techniques, e.g., using an automated DNA synthesizer.

In one embodiment, an isolated nucleic acid molecule of the invention comprises the nucleotide sequence shown in SEQ ID NO:46. The sequence of SEQ ID NO:46 corresponds to the human 8099 cDNA. This cDNA comprises sequences encoding the human 8099 polypeptide (i.e., “the coding region”, from nucleotides 180-2034) as well as 5′ untranslated sequences (nucleotides 1-179) and 3′ untranslated sequences (nucleotides 2035-2725). Alternatively, the nucleic acid molecule can comprise only the coding region of SEQ ID NO:46 (e.g., nucleotides 180-2034, corresponding to SEQ ID NO:48). Accordingly, in another embodiment, the isolated nucleic acid molecule comprises SEQ ID NO:48 and nucleotides 1-179 and 2035-2725 of SEQ ID NO:46. In yet another embodiment, the nucleic acid molecule consists of the nucleotide sequence set forth as SEQ ID NO:46 or 48.

In another embodiment, an isolated nucleic acid molecule of the invention comprises the nucleotide sequence shown in SEQ ID NO:49. The sequence of SEQ ID NO:49 corresponds to the human 46455 cDNA. This cDNA comprises sequences encoding the human 46455 polypeptide (i.e., “the coding region”, from nucleotides 376-1963) as well as 5′ untranslated sequences (nucleotides 1-375) and 3′ untranslated sequences (nucleotides 1964-2230). Alternatively, the nucleic acid molecule can comprise only the coding region of SEQ ID NO:49 (e.g., nucleotides 376-1963, corresponding to SEQ ID NO:51). Accordingly, in another embodiment, the isolated nucleic acid molecule comprises SEQ ID NO:51 and nucleotides 1-375 and 1964-2230 of SEQ ID NO:49 In yet another embodiment, the nucleic acid molecule consists of the nucleotide sequence set forth as SEQ ID NO:49 or SEQ ID NO:651.

In another embodiment, an isolated nucleic acid molecule of the invention comprises the nucleotide sequence shown in SEQ ID NO:52. This cDNA may comprise sequences encoding the human 54414 protein (e.g., the “coding region”, from nucleotides 225-3578), as well as 5′ untranslated sequence (nucleotides 1-224) and 3′ untranslated sequences (nucleotides 3579-4632) of SEQ ID NO:52. Alternatively, the nucleic acid molecule can comprise only the coding region of SEQ ID NO:52 (e.g., nucleotides 225-3578, corresponding to SEQ ID NO:54). Accordingly, in another embodiment, an isolated nucleic acid molecule of the invention comprises SEQ ID NO:54 and nucleotides 1-224 of SEQ ID NO:53. In yet another embodiment, the isolated nucleic acid molecule comprises SEQ ID NO:54 and nucleotides 3579-4632 of SEQ ID NO:52. In yet another embodiment, the nucleic acid molecule consists of the nucleotide sequence set forth as SEQ ID NO:52 or SEQ ID NO:54.

In still another embodiment, the cDNA may comprise sequences encoding the human 53763 protein (e.g., the “coding region”, from nucleotides 561-2474), as well as 5′ untranslated sequence (nucleotides 1-560) and 3′ untranslated sequences (nucleotides 2475-2847) of SEQ ID NO:55. Alternatively, the nucleic acid molecule can comprise only the coding region of SEQ ID NO:55 (e.g., nucleotides 561-2474, corresponding to SEQ ID NO:51). Accordingly, in another embodiment, an isolated nucleic acid molecule of the invention comprises SEQ ID NO:57 and nucleotides 1-560 of SEQ ID NO:55. In yet another embodiment, the isolated nucleic acid molecule comprises SEQ ID NO:57 and nucleotides 2475-2847 of SEQ ID NO:55. In yet another embodiment, the nucleic acid molecule consists of the nucleotide sequence set forth as SEQ ID NO:55 or SEQ ID NO:57.

In yet another embodiment, an isolated nucleic acid molecule of the invention comprises the nucleotide sequence shown in SEQ ID NO:58. The sequence of SEQ ID NO:58 corresponds to the human 67076 cDNA. This cDNA comprises sequences encoding the human 67076 polypeptide (i.e., “the coding region”, from nucleotides 524-3910) as well as 5′ untranslated sequences (nucleotides 1-523) and 3′ untranslated sequences (nucleotides 3911-6582). Alternatively, the nucleic acid molecule can comprise only the coding region of SEQ ID NO:58 (e.g., nucleotides 524-3910, corresponding to SEQ ID NO:60). Accordingly, in another embodiment, the isolated nucleic acid molecule comprises SEQ ID NO:60 and nucleotides 1-523 or 3911-6582 of SEQ ID NO:58. In yet another embodiment, the nucleic acid molecule consists of the nucleotide sequence set forth as SEQ ID NO:58 or SEQ ID NO:60.

In another embodiment, an isolated nucleic acid molecule of the invention comprises the nucleotide sequence shown in SEQ ID NO:61. The sequence of SEQ ID NO:61 corresponds to the human 67102 cDNA. This cDNA comprises sequences encoding the human 67102 polypeptide (i.e., “the coding region”, from nucleotides 274-4551) as well as 5′ untranslated sequences (nucleotides 1-273) and 3′ untranslated sequences (nucleotides 4552-6074). Alternatively, the nucleic acid molecule can comprise only the coding region of SEQ ID NO:61 (e.g., nucleotides 274-4551, corresponding to SEQ ID NO:63). Accordingly, in another embodiment, the isolated nucleic acid molecule comprises SEQ ID NO:63 and nucleotides 1-273 or 4552-6074 of SEQ ID NO:61. In yet another embodiment, the nucleic acid molecule consists of the nucleotide sequence set forth as SEQ ID NO:61 or SEQ ID NO:63.

In still another embodiment, an isolated nucleic acid molecule of the invention comprises the nucleotide sequence shown in SEQ ID NO:64. The sequence of SEQ ID NO:64 corresponds to the human 44181 cDNA. This cDNA comprises sequences encoding the human 44181 polypeptide (i.e., “the coding region”, from nucleotides 167-3697) as well as 5′ untranslated sequences (nucleotides 1-166) and 3′ untranslated sequences (nucleotides 3698-7221). Alternatively, the nucleic acid molecule can comprise only the coding region of SEQ ID NO:65 (e.g., nucleotides 167-3697, corresponding to SEQ ID NO:66). Accordingly, in another embodiment, the isolated nucleic acid molecule comprises SEQ ID NO:66 and nucleotides 1-166 or 3698-7221 of SEQ ID NO:64. In yet another embodiment, the nucleic acid molecule consists of the nucleotide sequence set forth as SEQ ID NO:64 or SEQ ID NO:66.

In yet another embodiment, an isolated nucleic acid molecule of the invention comprises the nucleotide sequence shown in SEQ ID NO:67. The sequence of SEQ ID NO:67 corresponds to the human 67084FL cDNA. This cDNA comprises sequences encoding the human 67084FL polypeptide (i.e., “the coding region”, from nucleotides 156-3407) as well as 5′ untranslated sequences (nucleotides 1-155) and 3′ untranslated sequences (nucleotides 3408-4198). Alternatively, the nucleic acid molecule can comprise only the coding region of SEQ ID NO:67 (e.g., nucleotides 156-3407, corresponding to SEQ ID NO:69). Accordingly, in another embodiment, the isolated nucleic acid molecule comprises SEQ ID NO:69 and nucleotides 1-155 or 3408-4198 of SEQ ID NO:67. In yet another embodiment, the nucleic acid molecule consists of the nucleotide sequence set forth as SEQ ID NO:67 or SEQ ID NO:69.

In a further embodiment, an isolated nucleic acid molecule of the invention comprises the nucleotide sequence shown in SEQ ID NO:70. The sequence of SEQ ID NO:70 corresponds to the human 67084alt cDNA. This cDNA comprises sequences encoding the human 67084alt polypeptide (i.e., “the coding region”, from nucleotides 156-3440) as well as 5′ untranslated sequences (nucleotides 1-155) and 3′ untranslated sequences (nucleotides 3441-4231). Alternatively, the nucleic acid molecule can comprise only the coding region of SEQ ID NO:70 (e.g., nucleotides 156-3440, corresponding to SEQ ID NO:72). Accordingly, in another embodiment, the isolated nucleic acid molecule comprises SEQ ID NO:72 and nucleotides 1-155 or 3441-4231 of SEQ ID NO:70. In yet another embodiment, the nucleic acid molecule consists of the nucleotide sequence set forth as SEQ ID NO:70 or SEQ ID NO:72.

In still another embodiment, an isolated nucleic acid molecule of the invention comprises a nucleic acid molecule which is a complement of the nucleotide sequence shown in sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, or a portion of any of these nucleotide sequences. A nucleic acid molecule which is complementary to the nucleotide sequence shown in SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, is one which is sufficiently complementary to the nucleotide sequence shown in SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, such that it can hybridize to the nucleotide sequence shown in SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 96%, 97%, 98%, 99% or more identical to the nucleotide sequence shown in SEQ ID NO:46, 48, 49, 51, 52, 54, 55, 57, 58, 60, 61, 63, 64, 66, 67, 69, 70, or 72 (e.g., to the entire length of the nucleotide sequence), or to the nucleotide sequence (e.g., the entire length of the nucleotide sequence) of the DNA insert of the plasmid deposited with ATCC as Accession Number ______, ______, ______, ______, or ______, or a portion of any of these nucleotide sequences. In one embodiment, a nucleic acid molecule of the present invention comprises a nucleotide sequence which is at least (or no greater than) 50-100, 100-250, 250-500, 500-750, 750-1000, 1000-1250, 1250-1500, 1500-1750, 1750-2000, 2000-2250, 2250-2500, 2500-2750, 2750-3000, 3000-3250, 3250-3500, 3500-3750, 3750-4000, 4000-4250, 4250-4500, 4500-4750, 4750-5000, 5000-5250, 5250-5500, 5500-5750, 5750-6000, 6000-6250, 6250-6500, 6500-6750, 6750-7000, 7000-7250, 7250-7500 or more nucleotides in length and hybridizes under stringent hybridization conditions to a complement of a nucleic acid molecule of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______.

Moreover, the nucleic acid molecule of the invention can comprise only a portion of the nucleic acid sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, for example, a fragment which can be used as a probe or primer or a fragment encoding a portion of a 8099, 46455, 54414, 53763, 67076, 67102, 44181, 67084FL, or 67084alt polypeptide, e.g., a biologically active portion of a 8099, 46455, 54414, 53763, 67076, 67102, 44181, 67084FL, or 67084alt polypeptide. The nucleotide sequence determined from the cloning of the 8099, 46455, 54414, 53763, 67076, 67102, 44181, 67084FL, or 67084alt gene allows for the generation of probes and primers designed for use in identifying and/or cloning other 8099, 46455, 54414, 53763, 67076, 67102, 44181, 67084FL, or 67084alt family members, as well as 8099, 46455, 54414, 53763, 67076, 67102, 44181, 67084FL, or 67084alt homologues from other species. The probe/primer typically comprises substantially purified oligonucleotide. The probe/primer (e.g., oligonucleotide) typically comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95, or 100 or more consecutive nucleotides of a sense sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, of an anti-sense sequence of SEQ ID NO:46, 48, 49, 51, 52, 54, 55, 57, 58, 60, 61, 63, 64, 66, 67, 69, 70, or 72 or the nucleotide sequence of the DNA insert of the plasmid deposited with ATCC as Accession Number ______, ______, ______, ______, or ______, or of a naturally occurring allelic variant or mutant of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______.

Exemplary probes or primers are at least 12, 15, 20, 25, 30, 35, 40, 45, 50, 55, 60, 65, 70, 75 or more nucleotides in length and/or comprise consecutive nucleotides of an isolated nucleic acid molecule described herein. Probes based on the 8099, 46455, 54414, 53763, 67076, 67102, 44181, 67084FL, or 67084alt nucleotide sequences can be used to detect (e.g., specifically detect) transcripts or genomic sequences encoding the same or homologous polypeptides. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of a 8099, 46455, 54414, 53763, 67076, 67102, 44181, 67084FL, or 67084alt sequence, e.g., a domain, region, site or other sequence described herein. The primers should be at least 5, 10, 20, 30, 40, 50, 60, 70, 80, 90, 100 or more nucleotides in length. Such probes can be used as a part of a diagnostic test kit for identifying cells or tissue which misexpress a 8099, 46455, 54414, 53763, 67076, 67102, 44181, 67084FL, or 67084alt polypeptide, such as by measuring a level of a 8099, 46455, 54414, 53763, 67076, 67102, 44181, 67084FL, or 67084alt-encoding nucleic acid in a sample of cells from a subject e.g., detecting 8099, 46455, 54414, 53763, 67076, 67102, 44181, 67084FL, or 67084alt mRNA levels or determining whether a genomic 8099, 46455, 54414, 53763, 67076, 67102, 44181, 67084FL, or 67084alt gene has been mutated or deleted.

A nucleic acid fragment encoding a “biologically active portion of a 8099, 46455, 54414, 53763, 67076, 67102, 44181, 67084FL, or 67084alt polypeptide” can be prepared by isolating a portion of the nucleotide sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, which encodes a polypeptide having a 8099, 46455, 54414, 53763, 67076, 67102, 44181, 67084FL, or 67084alt biological activity (the biological activities of the 8099, 46455, 54414, 53763, 67076, 67102, 44181, 67084FL, or 67084alt polypeptides are described herein), expressing the encoded portion of the 8099, 46455, 54414, 53763, 67076, 67102, 44181, 67084FL, or 67084alt polypeptide (e.g., by recombinant expression in vitro) and assessing the activity of the encoded portion of the 8099, 46455, 54414, 53763, 67076, 67102, 44181, 67084FL, or 67084alt polypeptide. In an exemplary embodiment, the nucleic acid molecule is at least 50-100, 100-250, 250-500, 500-750, 750-1000, 1000-1250, 1250-1500, 1500-1750, 1750-2000, 2000-2250, 2250-2500, 2500-2750, 2750-3000, 3000-3250, 3250-3500, 3500-3750, 3750-4000, 4000-4250, 4250-4500, 4500-4750, 4750-5000, 5000-5250, 5250-5500, 5500-5750, 5750-6000, 6000-6250, 6250-6500, 6500-6750, 6750-7000, 7000-7250, 7250-7500 or more nucleotides in length and encodes a polypeptide having a 8099, 46455, 54414, 53763, 67076, 67102, 44181, 67084FL, or 67084alt activity (as described herein).

The invention further encompasses nucleic acid molecules that differ from the nucleotide sequence shown in SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______. Such differences can be due to due to degeneracy of the genetic code, thus resulting in a nucleic acid which encodes the same 8099, 46455, 54414, 53763, 67076, 67102, 44181, 67084FL, or 67084alt polypeptides as those encoded by the nucleotide sequence shown in SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______. In another embodiment, an isolated nucleic acid molecule of the invention has a nucleotide sequence encoding a polypeptide having an amino acid sequence which differs by at least 1, but no greater than 5, 10, 20, 50 or 100 amino acid residues from the amino acid sequence shown in SEQ ID NO:47, 50, 53, 56, 59, 62, 65, 68, or 71, or the amino acid sequence encoded by the DNA insert of the plasmid deposited with the ATCC as Accession Number ______, ______, ______, ______, or ______. In yet another embodiment, the nucleic acid molecule encodes the amino acid sequence of human 8099, 46455, 54414, 53763, 67076, 67102, 44181, 67084FL, or 67084alt. If an alignment is needed for this comparison, the sequences should be aligned for maximum homology.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

Allelic variants result, for example, from DNA sequence polymorphisms within a population (e.g., the human population) that lead to changes in the amino acid sequences of the 8099, 46455, 54414, 53763, 67076, 67102, 44181, 67084FL, or 67084alt polypeptides. Such genetic polymorphism in the 8099, 46455, 54414, 53763, 67076, 67102, 44181, 67084FL, or 67084alt genes may exist among individuals within a population due to natural allelic variation. As used herein, the terms “gene” and “recombinant gene” refer to nucleic acid molecules which include an open reading frame encoding a 8099, 46455, 54414, 53763, 67076, 67102, 44181, 67084FL, or 67084alt polypeptide, preferably a mammalian 8099, 46455, 54414, 53763, 67076, 67102, 44181, 67084FL, or 67084alt polypeptide, and can further include non-coding regulatory sequences, and introns.

Accordingly, in one embodiment, the invention features isolated nucleic acid molecules which encode a naturally occurring allelic variant of a polypeptide comprising the amino acid sequence of SEQ ID NO:47, 50, 53, 56, 59, 62, 65, 68, or 71, or an amino acid sequence encoded by the DNA insert of the plasmid deposited with ATCC as Accession Number ______, ______, ______, ______, or ______, wherein the nucleic acid molecule hybridizes to a complement of a nucleic acid molecule comprising SEQ ID NO:46, 48, 49, 51, 52, 54, 55, 57, 58, 60, 61, 63, 64, 66, 67, 69, 70, or 72, for example, under stringent hybridization conditions.

Allelic variants of human 8099, 46455, 54414, 53763, 67076, 67102, 44181, 67084FL, or 67084alt include both functional and non-functional 8099, 46455, 54414, 53763, 67076, 67102, 44181, 67084FL, or 67084alt polypeptides.

Functional allelic variants are naturally occurring amino acid sequence variants of the human 8099 or 46455 polypeptides that have an 8099 or 46455 activity, e.g., maintain the ability to bind an 8099 or 46455 ligand or substrate and/or modulate sugar transport, or sugar homeostasis.

Functional allelic variants are naturally occurring amino acid sequence variants of the human 54414 or 53763 polypeptides that maintain the ability to, e.g., bind or interact with a 54414 or 53763 target molecule and/or modulate membrane excitability.

Functional allelic variants are naturally occurring amino acid sequence variants of the human 67076, 67102, 44181, 67084FL, or 67084alt polypeptides that have a 67076, 67102, 44181, 67084FL, or 67084alt activity, e.g, bind or interact with a 67076, 67102, 44181, 67084FL, or 67084alt substrate or target molecule, transport a 67076, 67102, 44181, 67084FL, or 67084alt substrate or target molecule across a cellular membrane, hydrolyze ATP, be phosphorylated or dephosphorylated, adopt an E1 conformation or an E2 conformation, and/or modulate cellular signaling, growth, proliferation, differentiation, absorption, or secretion.

Functional allelic variants will typically contain only conservative substitution of one or more amino acids of SEQ ID NO:47, 50, 53, 56, 59, 62, 65, 68, or 71, or substitution, deletion or insertion of non-critical residues in non-critical regions of the polypeptide.

Non-functional allelic variants are naturally occurring amino acid sequence variants of the human 8099 or 46455 polypeptides that do not have a 8099 or 46455 activity, e.g., maintain the ability to bind an 8099 or 46455 ligand or substrate and/or modulate sugar transport, or sugar homeostasis.

Non-functional allelic variants are naturally occurring amino acid sequence variants of the human 54414 or 53763 polypeptides that do not maintain the ability to, e.g., bind or interact with a 54414 or 53763 target molecule and/or modulate membrane excitability.

Non-functional allelic variants are naturally occurring amino acid sequence variants of the human 67076, 67102, 44181, 67084FL, or 67084alt polypeptides that do not have a 67076, 67102, 44181, 67084FL, or 67084alt activity, e.g., that do not have the ability to, e.g., bind or interact with a 67076, 67102, 44181, 67084FL, or 67084alt substrate or target molecule, transport a 67076, 67102, 44181, 67084FL, or 67084alt substrate or target molecule across a cellular membrane, hydrolyze ATP, be phosphorylated or dephosphorylated, adopt an E1 conformation or an E2 conformation, and/or modulate cellular signaling, growth, proliferation, differentiation, absorption, or secretion.

Non-functional allelic variants will typically contain a non-conservative substitution, a deletion, or insertion or premature truncation of the amino acid sequence of SEQ ID NO:47, 50, 53, 56, 59, 62, 65, 68, or 71, or a substitution, insertion or deletion in critical residues or critical regions.

The present invention further provides non-human orthologues of the human 8099, 46455, 54414, 53763, 67076, 67102, 44181, 67084FL, or 67084alt polypeptides. Orthologues of human 8099 or 46455 polypeptides are polypeptides that are isolated from non-human organisms and possess the same 8099 and/or 46455 activity, e.g., ligand binding and/or modulation of sugar transport mechanisms, as the human 8099 and/or 46455 polypeptide. Orthologues of the human 54414 or 53763 polypeptides are polypeptides that are isolated from non-human organisms and possess the same 54414 or 53763 target molecule binding mechanisms and/or ability to modulate membrane excitability of the human 54414 or 53763 polypeptides. Orthologues of human 67076, 67102, 44181, 67084FL, or 67084alt polypeptides are polypeptides that are isolated from non-human organisms and possess the same 67076, 67102, 44181, 67084FL, or 67084alt substrate or target molecule binding mechanisms, phospholipid transporting activity, ATPase activity, and/or modulation of cellular signaling mechanisms of the human 67076, 67102, 44181, 67084FL, or 67084alt proteins as the human 67076, 67102, 44181, 67084FL, or 67084alt polypeptides.

Orthologues of the human 8099, 46455, 54414, 53763, 67076, 67102, 44181, 67084FL, or 67084alt polypeptides can readily be identified as comprising an amino acid sequence that is substantially identical to SEQ ID NO:47, 50, 53, 56, 59, 62, 65, 68, or 71.

Moreover, nucleic acid molecules encoding other 8099, 46455, 54414, 53763, 67076, 67102, 44181, 67084FL, or 67084alt family members and, thus, which have a nucleotide sequence which differs from the 8099, 46455, 54414, 53763, 67076, 67102, 44181, 67084FL, or 67084alt sequences of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______ are intended to be within the scope of the invention. For example, another 8099, 46455, 54414, 53763, 67076, 67102, 44181, 67084FL, or 67084alt cDNA can be identified based on the nucleotide sequence of human 8099, 46455, 54414, 53763, 67076, 67102, 44181, 67084FL, or 67084alt. Moreover, nucleic acid molecules encoding 8099, 46455, 54414, 53763, 67076, 67102, 44181, 67084FL, or 67084alt polypeptides from different species, and which, thus, have a nucleotide sequence which differs from the 8099, 46455, 54414, 53763, 67076, 67102, 44181, 67084FL, or 67084alt sequences of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______ are intended to be within the scope of the invention. For example, a mouse 8099, 46455, 54414, 53763, 67076, 67102, 44181, 67084FL, or 67084alt cDNA can be identified based on the nucleotide sequence of a human 8099, 46455, 54414, 53763, 67076, 67102, 44181, 67084FL, or 67084alt.

Nucleic acid molecules corresponding to natural allelic variants and homologues of the 8099, 46455, 54414, 53763, 67076, 67102, 44181, 67084FL, or 67084alt cDNAs of the invention can be isolated based on their homology to the 8099, 46455, 54414, 53763, 67076, 67102, 44181, 67084FL, or 67084alt nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the 8099, 46455, 54414, 53763, 67076, 67102, 44181, 67084FL, or 67084alt cDNAs of the invention can further be isolated by mapping to the same chromosome or locus as the 8099, 46455, 54414, 53763, 67076, 67102, 44181, 67084FL, or 67084alt gene.

Orthologues, homologues and allelic variants can be identified using methods known in the art (e.g., by hybridization to an isolated nucleic acid molecule of the present invention, for example, under stringent hybridization conditions). In one embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______. In other embodiment, the nucleic acid is at least 50-100, 100-250, 250-500, 500-750, 750-1000, 1000-1250, 1250-1500, 1500-1750, 1750-2000, 2000-2250, 2250-2500, 2500-2750, 2750-3000, 3000-3250, 3250-3500, 3500-3750, 3750-4000, 4000-4250, 4250-4500, 4500-4750, 4750-5000, 5000-5250, 5250-5500, 5500-5750, 5750-6000, 6000-6250, 6250-6500, 6500-6750, 6750-7000, 7000-7250, 7250-7500 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

Preferably, an isolated nucleic acid molecule of the invention that hybridizes under stringent conditions to the sequence of SEQ ID NO:46, 48, 49, 51, 52, 54, 55, 57, 58, 60, 61, 63, 64, 66, 67, 69, 70, or 72, and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural polypeptide).

In addition to naturally-occurring allelic variants of the 8099, 46455, 54414, 53763, 67076, 67102, 44181, 67084FL, or 67084alt sequences that may exist in the population, the skilled artisan will further appreciate that changes can be introduced by mutation into the nucleotide sequences of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, thereby leading to changes in the amino acid sequence of the encoded 8099, 46455, 54414, 53763, 67076, 67102, 44181, 67084FL, or 67084alt polypeptides, without altering the functional ability of the 8099, 46455, 54414, 53763, 67076, 67102, 44181, 67084FL, or 67084alt polypeptides. For example, nucleotide substitutions leading to amino acid substitutions at “non-essential” amino acid residues can be made in the sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______. A “non-essential” amino acid residue is a residue that can be altered from the wild-type sequence of 8099, 46455, 54414, 53763, 67076, 67102, 44181, 67084FL, or 67084alt (e.g., the sequence of SEQ ID NO:47, 50, 53, 56, 59, 62, 65, 68, or 71) without altering the biological activity, whereas an “essential” amino acid residue is required for biological activity.

For example, amino acid residues that are conserved among the 8099 or 46455 polypeptides of the present invention, e.g., those present in a transmembrane domain and/or a sugar transporter family domain, are predicted to be particularly unamenable to alteration. Furthermore, additional amino acid residues that are conserved between the 8099 or 46455 polypeptides of the present invention and other members of the 8099 or 46455 family are not likely to be amenable to alteration.

Amino acid residues that are conserved among the 54414 or 53763 polypeptides of the present invention, e.g., those present in a P-loop or a pore domain, are predicted to be particularly unamenable to alteration. Furthermore, additional amino acid residues that are conserved between the 54414 or 53763 polypeptides of the present invention and other members of the potassium channel family are not likely to be amenable to alteration.

Amino acid residues that are conserved among the 67076, 67102, 44181, 67084FL, or 67084alt polypeptides of the present invention, e.g., those present in a E1-E2 ATPases phosphorylation site, are predicted to be particularly unamenable to alteration. Furthermore, additional amino acid residues that are conserved between the 67076, 67102, 44181, 67084FL, or 67084alt polypeptides of the present invention and other members of the phospholipid transporter family are not likely to be amenable to alteration.

Accordingly, another aspect of the invention pertains to nucleic acid molecules encoding 8099, 46455, 54414, 53763, 67076, 67102, 44181, 67084FL, or 67084alt polypeptides that contain changes in amino acid residues that are not essential for activity. Such 8099, 46455, 54414, 53763, 67076, 67102, 44181, 67084FL, or 67084alt polypeptides differ in amino acid sequence from SEQ ID NO:47, 50, 53, 56, 59, 62, 65, 68, or 71, yet retain biological activity. In one embodiment, the isolated nucleic acid molecule comprises a nucleotide sequence encoding a polypeptide, wherein the polypeptide comprises an amino acid sequence at least about 50%, 55%, 60%, 65%, 70%, 75%, 80%, 85%, 90%, 95%, 96%, 97%, 98%, 99% or more identical to SEQ ID NO:47, 50, 53, 56, 59, 62, 65, 68, or 71 (e.g., to the entire length of SEQ ID NO:47, 50, 53, 56, 59, 62, 65, 68, or 71).

An isolated nucleic acid molecule encoding a 8099, 46455, 54414, 53763, 67076, 67102, 44181, 67084FL, or 67084alt polypeptide identical to the polypeptide of SEQ ID NO:47, 50, 53, 56, 59, 62, 65, 68, or 71, can be created by introducing one or more nucleotide substitutions, additions or deletions into the nucleotide sequence of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______, such that one or more amino acid substitutions, additions or deletions are introduced into the encoded polypeptide. Mutations can be introduced into SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a 8099, 46455, 54414, 53763, 67076, 67102, 44181, 67084FL, or 67084alt polypeptide is preferably replaced with another amino acid residue from the same side chain family. Alternatively, in another embodiment, mutations can be introduced randomly along all or part of a 8099, 46455, 54414, 53763, 67076, 67102, 44181, 67084FL, or 67084alt coding sequence, such as by saturation mutagenesis, and the resultant mutants can be screened for 8099, 46455, 54414, 53763, 67076, 67102, 44181, 67084FL, or 67084alt biological activity to identify mutants that retain activity. Following mutagenesis of SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, ______, ______, ______, ______, or ______, the encoded polypeptide can be expressed recombinantly and the activity of the polypeptide can be determined.

In a preferred embodiment, a mutant 8099 and/or 46455 polypeptide can be assayed for the ability to (1) bind a monosaccharide, e.g., D-glucose, D-fructose, D-galactose, and/or mannose, (2) transport monosaccharides across a cell membrane, (3) influence insulin and/or glucagon secretion, (4) maintain sugar homeostasis in a cell, (5) function as a neuronal transporter, and (6) mediate trans-epithelial movement in a cell.

In another preferred embodiment, a mutant 54414 and/or 53763 protein can be assayed for the ability to (i) interact with a 54414 and/or 53763 substrate (e.g., a potassium ion or a cyclic nucleotide); (ii) conduct or transport a 54414 and/or 53763 substrate across a cellular membrane; (iii) interact with a second non-54414 and/or 53763 protein (e.g., a 54414 and/or 53763 polypeptide or a 54414 and/or 53763-potassium channel subunit); (iv) modulate (e.g., maintain and/or rectify) membrane potentials; (v) regulate target molecule availability or activity; (vi) modulate intra- or intercellular signaling and/or gene transcription (e.g., either directly or indirectly); (viii) generate outwardly rectifying currents; (viii) modulate membrane excitability; (ix) modulate the release of neurotransmitters; (x) regulate contractility (e.g., of smooth muscle cells), secretion, and/or synaptic transmission; and/or (xi) modulate processes which underlie learning and memory.

In a further preferred embodiment, a mutant 54414 protein can be assayed for the ability to (i) interact with maxi-K potassium channels (i.e., large conductance channels, in particular Slo); (ii) modulate maxi-K potassium channel activity (e.g., Slo-mediated activities); (iii) generate intermediate conductance channels; and/or (iv) regulate contractility (e.g., of smooth muscle cells), secretion, and/or synaptic transmission, in particular, via modulation of Slo.

In still a further preferred embodiment, a mutant 53763 protein can be assayed for the ability to (i) interact with Shaker (Sh) potassium channels and/or channel subunits; (ii) modulate Shaker (Sh) potassium channel activity (e.g., termination of prolonged membrane depolarization); and/or (iii) modulation of high voltage activating channel activity and/or inactivating channel activity, and the like.

In yet another preferred embodiment, a mutant 67076, 67102, 44181, 67084FL, and/or 67084alt polypeptide can be assayed for the ability to (i) interact with a 67076, 67102, 44181, 67084FL, or 67084alt substrate or target molecule (e.g., a phospholipid, ATP, or a non-67076, 67102, 44181, 67084FL, or 67084alt protein); (ii) transport a 67076, 67102, 44181, 67084FL, or 67084alt substrate or target molecule (e.g., an aminophospholipid such as phosphatidylserine or phosphatidylethanolamine) from one side of a cellular membrane to the other; (iii) be phosphorylated or dephosphorylated; (iv) adopt an E1 conformation or an E2 conformation; (v) convert a 67076, 67102, 44181, 67084FL, or 67084alt substrate or target molecule to a product (e.g., hydrolysis of ATP); (vi) interact with a second non-67076, 67102, 44181, 67084FL, or 67084alt protein; (vii) modulate substrate or target molecule location (e.g., modulation of phospholipid location within a cell and/or location with respect to a cellular membrane); (viii) maintain aminophospholipid gradients; (ix) modulate intra- or intercellular signaling and/or gene transcription (e.g., either directly or indirectly); and/or (x) modulate cellular proliferation, growth, differentiation, apoptosis, absorption, or secretion.

In addition to the nucleic acid molecules encoding 8099, 46455, 54414, 53763, 67076, 67102, 44181, 67084FL, or 67084alt polypeptides described above, another aspect of the invention pertains to isolated nucleic acid molecules which are antisense thereto. In an exemplary embodiment, the invention provides an isolated nucleic acid molecule which is antisense to a 8099, 46455, 54414, 53763, 67076, 67102, 44181, 67084FL, or 67084alt nucleic acid molecule (e.g., is antisense to the coding strand of a 8099, 46455, 54414, 53763, 67076, 67102, 44181, 67084FL, or 67084alt nucleic acid molecule). An “antisense” nucleic acid comprises a nucleotide sequence which is complementary to a “sense” nucleic acid encoding a polypeptide, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 8099, 46455, 54414, 53763, 67076, 67102, 44181, 67084FL, or 67084alt coding strand, or to only a portion thereof. In one embodiment, an antisense nucleic acid molecule is antisense to a “coding region” of the coding strand of a nucleotide sequence encoding 8099, 46455, 54414, 53763, 67076, 67102, 44181, 67084FL, or 67084alt. The term “coding region” refers to the region of the nucleotide sequence comprising codons which are translated into amino acid residues (e.g., the coding regions of human 8099, 46455, 54414, 53763, 67076, 67102, 44181, 67084FL, and 67084alt correspond to SEQ ID NO:48, 51, 54, 57, 60, 63, 66, 69, and 72, respectively). In another embodiment, the antisense nucleic acid molecule is antisense to a “noncoding region” of the coding strand of a nucleotide sequence encoding 8099, 46455, 54414, 53763, 67076, 67102, 44181, 67084FL, or 67084alt. The term “noncoding region” refers to 5′ and 3′ sequences which flank the coding region that are not translated into amino acids (i.e., also referred to as 5′ and 3′ untranslated regions).

Given the coding strand sequences encoding 8099, 46455, 54414, 53763, 67076, 67102, 44181, 67084FL, or 67084alt disclosed herein (e.g., SEQ ID NO:48, 51, 54, 57, 60, 63, 66, 69, and 72), antisense nucleic acids of the invention can be designed according to the rules of Watson and Crick base pairing. The antisense nucleic acid molecule can be complementary to the entire coding region of 8099, 46455, 54414, 53763, 67076, 67102, 44181, 67084FL, or 67084alt mRNA, but more preferably is an oligonucleotide which is antisense to only a portion of the coding or noncoding region of 8099, 46455, 54414, 53763, 67076, 67102, 44181, 67084FL, or 67084alt mRNA. For example, the antisense oligonucleotide can be complementary to the region surrounding the translation start site of 8099, 46455, 54414, 53763, 67076, 67102, 44181, 67084FL, or 67084alt mRNA (e.g., between the −10 and +10 regions of the start site of a gene nucleotide sequence). An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a 8099, 46455, 54414, 53763, 67076, 67102, 44181, 67084FL, or 67084alt polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intra-cellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave 8099, 46455, 54414, 53763, 67076, 67102, 44181, 67084FL, or 67084alt mRNA transcripts to thereby inhibit translation of 8099, 46455, 54414, 53763, 67076, 67102, 44181, 67084FL, or 67084alt mRNA. A ribozyme having specificity for a 8099-, 46455-, 54414-, 53763-, 67076-, 67102-, 44181-, 67084FL-, or 67084alt-encoding nucleic acid can be designed based upon the nucleotide sequence of a 8099, 46455, 54414, 53763, 67076, 67102, 44181, 67084FL, or 67084alt cDNA disclosed herein (i.e., SEQ ID NO:46, 48, 49, 51, 52, 54, 55, 57, 58, 60, 61, 63, 64, 66, 67, 69, 70, or 72, or the nucleotide sequence of the DNA insert of the plasmid deposited with ATCC as Accession Number ______, ______, ______, ______, or ______). For example, a derivative of a Tetrahymena L-19 IVS RNA can be constructed in which the nucleotide sequence of the active site is complementary to the nucleotide sequence to be cleaved in a 8099-, 46455-, 54414-, 53763-, 67076-, 67102-, 44181-, 67084FL-, or 67084alt-encoding mRNA. See, e.g., Cech et al. U.S. Pat. No. 4,987,071; and Cech et al. U.S. Pat. No. 5,116,742. Alternatively, 8099, 46455, 54414, 53763, 67076, 67102, 44181, 67084FL, or 67084alt mRNA can be used to select a catalytic RNA having a specific ribonuclease activity from a pool of RNA molecules. See, e.g., Bartel, D. and Szostak, J. W. (1993) Science 261:1411-1418.

Alternatively, 8099, 46455, 54414, 53763, 67076, 67102, 44181, 67084FL, or 67084alt gene expression can be inhibited by targeting nucleotide sequences complementary to the regulatory region of the 8099, 46455, 54414, 53763, 67076, 67102, 44181, 67084FL, or 67084alt (e.g., the 8099, 46455, 54414, 53763, 67076, 67102, 44181, 67084FL, or 67084alt promoter and/or enhancers) to form triple helical structures that prevent transcription of the 8099, 46455, 54414, 53763, 67076, 67102, 44181, 67084FL, or 67084alt gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the 8099, 46455, 54414, 53763, 67076, 67102, 44181, 67084FL, or 67084alt nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of 8099, 46455, 54414, 53763, 67076, 67102, 44181, 67084FL, or 67084alt nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 8099, 46455, 54414, 53763, 67076, 67102, 44181, 67084FL, or 67084alt nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of 8099, 46455, 54414, 53763, 67076, 67102, 44181, 67084FL, or 67084alt can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 8099, 46455, 54414, 53763, 67076, 67102, 44181, 67084FL, or 67084alt nucleic acid molecules can be generated which may combine the advantageous properties of PNA and DNA. Such chimeras allow DNA recognition enzymes, (e.g., RNase H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl) amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous 8099, 46455, 54414, 53763, 67076, 67102, 44181, 67084FL, or 67084alt gene within a cell line or microorganism may be modified by inserting a heterologous DNA regulatory element into the genome of a stable cell line or cloned microorganism such that the inserted regulatory element is operatively linked with the endogenous 8099, 46455, 54414, 53763, 67076, 67102, 44181, 67084FL, or 67084alt gene. For example, an endogenous 8099, 46455, 54414, 53763, 67076, 67102, 44181, 67084FL, or 67084alt gene which is normally “transcriptionally silent”, i.e., a 8099, 46455, 54414, 53763, 67076, 67102, 44181, 67084FL, or 67084alt gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous 8099, 46455, 54414, 53763, 67076, 67102, 44181, 67084FL, or 67084alt gene may be activated by insertion of a promiscuous regulatory element that works across cell types.

A heterologous regulatory element may be inserted into a stable cell line or cloned microorganism, such that it is operatively linked with an endogenous 8099, 46455, 54414, 53763, 67076, 67102, 44181, 67084FL, or 67084alt gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated 8099, 46455, 54414, 53763, 67076, 67102, 44181, 67084FL, or 67084alt Polypeptides and Anti-8099, 46455, 54414, 53763, 67076, 67102, 44181, 67084FL, or 67084alt Antibodies

One aspect of the invention pertains to isolated 8099, 46455, 54414, 53763, 67076, 67102, 44181, 67084FL, or 67084alt or recombinant polypeptides and polypeptides, and biologically active portions thereof, as well as polypeptide fragments suitable for use as immunogens to raise anti-8099, 46455, 54414, 53763, 67076, 67102, 44181, 67084FL, or 67084alt antibodies. In one embodiment, native 8099, 46455, 54414, 53763, 67076, 67102, 44181, 67084FL, or 67084alt polypeptides can be isolated from cells or tissue sources by an appropriate purification scheme using standard protein purification techniques. In another embodiment, 8099, 46455, 54414, 53763, 67076, 67102, 44181, 67084FL, or 67084alt polypeptides are produced by recombinant DNA techniques. Alternative to recombinant expression, a 8099, 46455, 54414, 53763, 67076, 67102, 44181, 67084FL, or 67084alt polypeptide or polypeptide can be synthesized chemically using standard peptide synthesis techniques.

An “isolated” or “purified” polypeptide or biologically active portion thereof is substantially free of cellular material or other contaminating proteins from the cell or tissue source from which the 8099, 46455, 54414, 53763, 67076, 67102, 44181, 67084FL, or 67084alt polypeptide is derived, or substantially free from chemical precursors or other chemicals when chemically synthesized. The language “substantially free of cellular material” includes preparations of 8099, 46455, 54414, 53763, 67076, 67102, 44181, 67084FL, or 67084alt polypeptide in which the polypeptide is separated from cellular components of the cells from which it is isolated or recombinantly produced. In one embodiment, the language “substantially free of cellular material” includes preparations of 8099, 46455, 54414, 53763, 67076, 67102, 44181, 67084FL, or 67084alt polypeptide having less than about 30% (by dry weight) of non-8099, 46455, 54414, 53763, 67076, 67102, 44181, 67084FL, or 67084alt polypeptide (also referred to herein as a “contaminating protein”), more preferably less than about 20% of non-8099, 46455, 54414, 53763, 67076, 67102, 44181, 67084FL, or 67084alt polypeptide, still more preferably less than about 10% of non-8099, 46455, 54414, 53763, 67076, 67102, 44181, 67084FL, or 67084alt polypeptide, and most preferably less than about 5% non-8099, 46455, 54414, 53763, 67076, 67102, 44181, 67084FL, or 67084alt polypeptide. When the 8099, 46455, 54414, 53763, 67076, 67102, 44181, 67084FL, or 67084alt polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of 8099, 46455, 54414, 53763, 67076, 67102, 44181, 67084FL, or 67084alt polypeptide in which the polypeptide is separated from chemical precursors or other chemicals which are involved in the synthesis of the polypeptide. In one embodiment, the language “substantially free of chemical precursors or other chemicals” includes preparations of 8099, 46455, 54414, 53763, 67076, 67102, 44181, 67084FL, or 67084alt polypeptide having less than about 30% (by dry weight) of chemical precursors or non-8099, 46455, 54414, 53763, 67076, 67102, 44181, 67084FL, or 67084alt chemicals, more preferably less than about 20% chemical precursors or non-8099, 46455, 54414, 53763, 67076, 67102, 44181, 67084FL, or 67084alt chemicals, still more preferably less than about 10% chemical precursors or non-8099, 46455, 54414, 53763, 67076, 67102, 44181, 67084FL, or 67084alt chemicals, and most preferably less than about 5% chemical precursors or non-8099, 46455, 54414, 53763, 67076, 67102, 44181, 67084FL, or 67084alt chemicals.

As used herein, a “biologically active portion” of a 8099, 46455, 54414, 53763, 67076, 67102, 44181, 67084FL, or 67084alt polypeptide includes a fragment of a 8099, 46455, 54414, 53763, 67076, 67102, 44181, 67084FL, or 67084alt polypeptide which participates in an interaction between a 8099, 46455, 54414, 53763, 67076, 67102, 44181, 67084FL, or 67084alt molecule and a non-8099, 46455, 54414, 53763, 67076, 67102, 44181, 67084FL, or 67084alt molecule (e.g., a 8099, 46455, 54414, 53763, 67076, 67102, 44181, 67084FL, or 67084alt substrate). Biologically active portions of a 8099, 46455, 54414, 53763, 67076, 67102, 44181, 67084FL, or 67084alt polypeptide include peptides comprising amino acid sequences sufficiently identical to or derived from the amino acid sequence of the 8099, 46455, 54414, 53763, 67076, 67102, 44181, 67084FL, or 67084alt polypeptide, e.g., the amino acid sequence shown in SEQ ID NO:47, 50, 53, 56, 59, 62, 65, 68, or 71, which include less amino acids than the full length 8099, 46455, 54414, 53763, 67076, 67102, 44181, 67084FL, or 67084alt polypeptides, and exhibit at least one activity of a 8099, 46455, 54414, 53763, 67076, 67102, 44181, 67084FL, or 67084alt polypeptide.

Typically, biologically active portions of a 8099 or 46455 polypeptide comprise a domain or motif with at least one activity of the 8099 or 46455 polypeptide, e.g., modulating sugar transport mechanisms. A biologically active portion of an 8099 polypeptide can be a polypeptide which is, for example, 25, 30, 35, 40, 45, 50, 75, 100, 125, 150, 175, 200, 225, 250, 275, 300, 325, 350, 375, 400, 425, 450, 475, 500, 525, 550, 600 or more amino acids in length. A biologically active portion of an 46455 polypeptide can be a polypeptide which is, for example, 25, 30, 35, 40, 45, 50, 75, 100, 125, 150, 175, 200, 225, 250, 275, 300, 325, 350, 375, 400, 425, 450, 475, 500, 525 or more amino acids in length. Biologically active portions of an 8099 and/or an 46455 polypeptide can be used as targets for developing agents which modulate an 8099 or 46455 mediated activity, e.g., a sugar transport mechanism.

In one embodiment, a biologically active portion of an 8099 or an 46455 polypeptide comprises at least one transmembrane domain. It is to be understood that a preferred biologically active portion of an 8099 or an 46455 polypeptide of the present invention comprises at least one or more of the following domains: a transmembrane domain and/or a sugar transporter family domain. Moreover, other biologically active portions, in which other regions of the polypeptide are deleted, can be prepared by recombinant techniques and evaluated for one or more of the functional activities of a native 8099 or 46455 polypeptide.

Moreover, biologically active portions of a 54414 or 53763 polypeptide comprise a domain or motif with at least one activity of the 54414 or 53763 polypeptide, e.g., modulation of intra- or inter-cellular signaling and/or gene expression, and/or modulate membrane excitability. A biologically active portion of a 54414 or 53763 polypeptide can be a polypeptide which is, for example, 10, 25, 50, 75, 100, 125, 150 or more amino acids in length. Biologically active portions of a 54414 or 53763 polypeptide can be used as targets for developing agents which modulate a 54414 or 53763 mediated activity, e.g., modulation of intra- or inter-cellular signaling and/or gene expression, and/or modulate membrane excitability.

In one embodiment, a biologically active portion of a 54414 or 53763 polypeptide comprises at least one transmembrane domain and/or a pore domain. Moreover, other biologically active portions, in which other regions of the protein are deleted, can be prepared by recombinant techniques and evaluated for one or more of the functional activities of a native 54414 or 53763 polypeptide.

Biologically active portions of a 67076, 67102, 44181, 67084FL, or 67084alt polypeptide comprise a domain or motif with at least one activity of the 67076, 67102, 44181, 67084FL, or 67084alt polypeptide, e.g., the ability to interact with a 67076, 67102, 44181, 67084FL, or 67084alt substrate or target molecule (e.g., a phospholipid; ATP; a non-67076, 67102, 44181, 67084FL, or 67084alt protein; or another 67076, 67102, 44181, 67084FL, or 67084alt protein or subunit); the ability to transport a 67076, 67102, 44181, 67084FL, or 67084alt substrate or target molecule (e.g., a phospholipid) from one side of a cellular membrane to the other; the ability to be phosphorylated or dephosphorylated; the ability to adopt an E1 conformation or an E2 conformation; the ability to convert a 67076, 67102, 44181, 67084FL, or 67084alt substrate or target molecule to a product (e.g., the ability to hydrolyze ATP); the ability to interact with a second non-67076, 67102, 44181, 67084FL, or 67084alt protein; the ability to modulate intra- or inter-cellular signaling and/or gene transcription (e.g., either directly or indirectly); the ability to modulate cellular growth, proliferation, differentiation, absorption, and/or secretion. A biologically active portion of a 67076, 67102, 44181, 67084FL, or 67084alt polypeptide can be a polypeptide which is, for example, 10, 25, 50, 75, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550 or more amino acids in length. Biologically active portions of a 67076, 67102, 44181, 67084FL, or 67084alt polypeptide can be used as targets for developing agents which modulate a 67076, 67102, 44181, 67084FL, or 67084alt mediated activity, e.g., modulating transport of biological molecules across membranes.

In one embodiment, a biologically active portion of a 67076, 67102, 44181, 67084FL, or 67084alt polypeptide comprises at least one at least one or more of the following domains, sites, or motifs: a transmembrane domain, an N-terminal large extramembrane domain, a C-terminal large extramembrane domain, an E1-E2 ATPases phosphorylation site, a P-type ATPase sequence 1 motif, a P-type ATPase sequence 2 motif, a P-type ATPase sequence 3 motif, and/or one or more phospholipid transporter specific amino acid resides. Moreover, other biologically active portions, in which other regions of the polypeptide are deleted, can be prepared by recombinant techniques and evaluated for one or more of the functional activities of a native 67076, 67102, 44181, 67084FL, or 67084alt polypeptide.

Another aspect of the invention features fragments of the polypeptide having the amino acid sequence of SEQ ID NO:47, 50, 53, 56, 59, 62, 65, 68, or 71, for example, for use as immunogens. In one embodiment, a fragment comprises at least 5 amino acids (e.g., contiguous or consecutive amino acids) of the amino acid sequence of SEQ ID NO:47, 50, 53, 56, 59, 62, 65, 68, or 71, or an amino acid sequence encoded by the DNA insert of the plasmid deposited with the ATCC as Accession Number ______, ______, ______, ______, or ______. In another embodiment, a fragment comprises at least 10, 15, 20, 25, 30, 35, 40, 45, 50 or more amino acids (e.g., contiguous or consecutive amino acids) of the amino acid sequence of SEQ ID NO:47, 50, 53, 56, 59, 62, 65, 68, or 71, or an amino acid sequence encoded by the DNA insert of the plasmid deposited with the ATCC as Accession Number ______, ______, ______, ______, or ______.

In a preferred embodiment, a 8099, 46455, 54414, 53763, 67076, 67102, 44181, 67084FL, or 67084alt polypeptide has an amino acid sequence shown in SEQ ID NO:47, 50, 53, 56, 59, 62, 65, 68, or 71. In other embodiments, the 8099, 46455, 54414, 53763, 67076, 67102, 44181, 67084FL, or 67084alt polypeptide is substantially identical to SEQ ID NO:47, 50, 53, 56, 59, 62, 65, 68, or 71, and retains the functional activity of the polypeptide of SEQ ID NO:47, 50, 53, 56, 59, 62, 65, 68, or 71, yet differs in amino acid sequence due to natural allelic variation or mutagenesis, as described in detail in subsection I above. In another embodiment, the 8099, 46455, 54414, 53763, 67076, 67102, 44181, 67084FL, or 67084alt polypeptide is a polypeptide which comprises an amino acid sequence at least about 50%, 55%, 60%, 65%, 70%, 75%, 80%, 85%, 90%, 95%, 96%, 97%, 98%, 99% or more identical to SEQ ID NO:47, 50, 53, 56, 59, 62, 65, 68, or 71.

In another embodiment, the invention features a 8099, 46455, 54414, 53763, 67076, 67102, 44181, 67084FL, or 67084alt polypeptide which is encoded by a nucleic acid molecule consisting of a nucleotide sequence at least about 50%, 55%, 60%, 65%, 70%, 75%, 80%, 85%, 90%, 95%, 96%, 97%, 98%, 99% or more identical to a nucleotide sequence of SEQ ID NO:46, 48, 49, 51, 52, 54, 55, 57, 58, 60, 61, 63, 64, 66, 67, 69, 70, or 72, or a complement thereof. This invention further features a 8099, 46455, 54414, 53763, 67076, 67102, 44181, 67084FL, or 67084alt polypeptide which is encoded by a nucleic acid molecule consisting of a nucleotide sequence which hybridizes under stringent hybridization conditions to a complement of a nucleic acid molecule comprising the nucleotide sequence of SEQ ID NO:46, 48, 49, 51, 52, 54, 55, 57, 58, 60, 61, 63, 64, 66, 67, 69, 70, or 72, or a complement thereof.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 8099 amino acid sequence of SEQ ID NO:47 having 617 amino acid residues, at least 185, preferably at least 246, more preferably at least 308, more preferably at least 370, even more preferably at least 431, and even more preferably at least 493 or 555 or more amino acid residues are aligned. In another preferred embodiment, the sequences being aligned for comparison purposes are globally aligned and percent identity is determined over the entire length of the sequences aligned. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at the Accelrys website), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of E. Meyers and W. Miller (Comput. Appl. Biosci., 4:11-17 (1988)) which has been incorporated into the ALIGN program (version 2.0 or version 2.0U), using a PAM120 weight residue table, a gap length penalty of 12 and a gap penalty of 4.

The nucleic acid and polypeptide sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 8099, 46455, 54414, 53763, 67076, 67102, 44181, 67084FL, or 67084alt nucleic acid molecules of the invention. BLAST protein searches can be performed with the XBLAST program, score=100, wordlength=3, and a Blosum62 matrix to obtain amino acid sequences homologous to 8099, 46455, 54414, 53763, 67076, 67102, 44181, 67084FL, or 67084alt polypeptide molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See the National Center for Biotechnology website.

The invention also provides 8099, 46455, 54414, 53763, 67076, 67102, 44181, 67084FL, or 67084alt chimeric or fusion proteins. As used herein, a 8099, 46455, 54414, 53763, 67076, 67102, 44181, 67084FL, or 67084alt “chimeric protein” or “fusion protein” comprises a 8099, 46455, 54414, 53763, 67076, 67102, 44181, 67084FL, or 67084alt polypeptide operatively linked to a non-8099, 46455, 54414, 53763, 67076, 67102, 44181, 67084FL, or 67084alt polypeptide. A “8099, 46455, 54414, 53763, 67076, 67102, 44181, 67084FL, or 67084alt polypeptide” refers to a polypeptide having an amino acid sequence corresponding to a 8099, 46455, 54414, 53763, 67076, 67102, 44181, 67084FL, or 67084alt polypeptide whereas a “non-8099, 46455, 54414, 53763, 67076, 67102, 44181, 67084FL, or 67084alt polypeptide” refers to a polypeptide having an amino acid sequence corresponding to a polypeptide which is not substantially homologous to the 8099, 46455, 54414, 53763, 67076, 67102, 44181, 67084FL, or 67084alt polypeptides, respectively, e.g., a polypeptide which is different from the 8099, 46455, 54414, 53763, 67076, 67102, 44181, 67084FL, or 67084alt polypeptide and which is derived from the same or a different organism. Within a 8099, 46455, 54414, 53763, 67076, 67102, 44181, 67084FL, or 67084alt fusion protein the 8099, 46455, 54414, 53763, 67076, 67102, 44181, 67084FL, or 67084alt polypeptide can correspond to all or a portion of a 8099, 46455, 54414, 53763, 67076, 67102, 44181, 67084FL, or 67084alt polypeptide. In a preferred embodiment, a 8099, 46455, 54414, 53763, 67076, 67102, 44181, 67084FL, or 67084alt fusion protein comprises at least one biologically active portion of a 8099, 46455, 54414, 53763, 67076, 67102, 44181, 67084FL, or 67084alt polypeptide. In another preferred embodiment, a 8099, 46455, 54414, 53763, 67076, 67102, 44181, 67084FL, or 67084alt fusion protein comprises at least two biologically active portions of a 8099, 46455, 54414, 53763, 67076, 67102, 44181, 67084FL, or 67084alt polypeptide. Within the fusion protein, the term “operatively linked” is intended to indicate that the 8099, 46455, 54414, 53763, 67076, 67102, 44181, 67084FL, or 67084alt polypeptide and the non-8099, 46455, 54414, 53763, 67076, 67102, 44181, 67084FL, or 67084alt polypeptide are fused in-frame to each other. The non-8099, 46455, 54414, 53763, 67076, 67102, 44181, 67084FL, or 67084alt polypeptide can be fused to the N-terminus or C-terminus of the 8099, 46455, 54414, 53763, 67076, 67102, 44181, 67084FL, or 67084alt polypeptide.

For example, in one embodiment, the fusion protein is a GST-8099, -46455, -54414, -53763, -67076, -67102, -44181, -67084FL, or -67084alt fusion protein in which the 8099, 46455, 54414, 53763, 67076, 67102, 44181, 67084FL, or 67084alt sequences are fused to the C-terminus of the GST sequences. Such fusion proteins can facilitate the purification of recombinant 8099, 46455, 54414, 53763, 67076, 67102, 44181, 67084FL, or 67084alt.

In another embodiment, the fusion protein is a 8099, 46455, 54414, 53763, 67076, 67102, 44181, 67084FL, or 67084alt polypeptide containing a heterologous signal sequence at its N-terminus. In certain host cells (e.g., mammalian host cells), expression and/or secretion of 8099, 46455, 54414, 53763, 67076, 67102, 44181, 67084FL, or 67084alt can be increased through the use of a heterologous signal sequence.

The 8099, 46455, 54414, 53763, 67076, 67102, 44181, 67084FL, or 67084alt fusion proteins of the invention can be incorporated into pharmaceutical compositions and administered to a subject in vivo. The 8099, 46455, 54414, 53763, 67076, 67102, 44181, 67084FL, or 67084alt fusion proteins can be used to affect the bioavailability of a 8099, 46455, 54414, 53763, 67076, 67102, 44181, 67084FL, or 67084alt substrate. Use of 8099, 46455, 54414, 53763, 67076, 67102, 44181, 67084FL, or 67084alt fusion proteins may be useful therapeutically for the treatment of disorders caused by, for example, (i) aberrant modification or mutation of a gene encoding a 8099, 46455, 54414, 53763, 67076, 67102, 44181, 67084FL, or 67084alt polypeptide; (ii) mis-regulation of the 8099, 46455, 54414, 53763, 67076, 67102, 44181, 67084FL, or 67084alt gene; and (iii) aberrant post-translational modification of a 8099, 46455, 54414, 53763, 67076, 67102, 44181, 67084FL, or 67084alt polypeptide.

Moreover, the 8099, 46455, 54414, 53763, 67076, 67102, 44181, 67084FL, or 67084alt-fusion proteins of the invention can be used as immunogens to produce anti-8099, anti-46455, anti-54414, anti-53763, anti-67076, anti-67102, anti-44181, anti-67084FL, and/or anti-67084alt antibodies in a subject, to purify 8099, 46455, 54414, 53763, 67076, 67102, 44181, 67084FL, or 67084alt ligands and in screening assays to identify molecules which inhibit the interaction with or transport of 8099, 46455, 54414, 53763, 67076, 67102, 44181, 67084FL, or 67084alt with a 8099, 46455, 54414, 53763, 67076, 67102, 44181, 67084FL, or 67084alt substrate.

Preferably, a 8099, 46455, 54414, 53763, 67076, 67102, 44181, 67084FL, or 67084alt chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 8099, 46455, 54414, 53763, 67076, 67102, 44181, 67084FL, or 67084alt-encoding nucleic acid can be cloned into such an expression vector such that the fusion moiety is linked in-frame to the 8099, 46455, 54414, 53763, 67076, 67102, 44181, 67084FL, or 67084alt polypeptide.

The present invention also pertains to variants of the 8099, 46455, 54414, 53763, 67076, 67102, 44181, 67084FL, or 67084alt polypeptides which function as either 8099, 46455, 54414, 53763, 67076, 67102, 44181, 67084FL, or 67084alt agonists (mimetics) or as 8099, 46455, 54414, 53763, 67076, 67102, 44181, 67084FL, or 67084alt antagonists. Variants of the 8099, 46455, 54414, 53763, 67076, 67102, 44181, 67084FL, or 67084alt polypeptides can be generated by mutagenesis, e.g., discrete point mutation or truncation of a 8099, 46455, 54414, 53763, 67076, 67102, 44181, 67084FL, or 67084alt polypeptide. An agonist of the 8099, 46455, 54414, 53763, 67076, 67102, 44181, 67084FL, or 67084alt polypeptides can retain substantially the same, or a subset, of the biological activities of the naturally occurring form of a 8099, 46455, 54414, 53763, 67076, 67102, 44181, 67084FL, or 67084alt polypeptide. An antagonist of a 8099, 46455, 54414, 53763, 67076, 67102, 44181, 67084FL, or 67084alt polypeptide can inhibit one or more of the activities of the naturally occurring form of the 8099, 46455, 54414, 53763, 67076, 67102, 44181, 67084FL, or 67084alt polypeptide by, for example, competitively modulating a 8099, 46455, 54414, 53763, 67076, 67102, 44181, 67084FL, or 67084alt-mediated activity of a 8099, 46455, 54414, 53763, 67076, 67102, 44181, 67084FL, or 67084alt polypeptide. Thus, specific biological effects can be elicited by treatment with a variant of limited function. In one embodiment, treatment of a subject with a variant having a subset of the biological activities of the naturally occurring form of the polypeptide has fewer side effects in a subject relative to treatment with the naturally occurring form of the 8099, 46455, 54414, 53763, 67076, 67102, 44181, 67084FL, or 67084alt polypeptide.

In one embodiment, variants of a 8099, 46455, 54414, 53763, 67076, 67102, 44181, 67084FL, or 67084alt polypeptide which function as either 8099, 46455, 54414, 53763, 67076, 67102, 44181, 67084FL, or 67084alt agonists (mimetics) or as 8099, 46455, 54414, 53763, 67076, 67102, 44181, 67084FL, or 67084alt antagonists can be identified by screening combinatorial libraries of mutants, e.g., truncation mutants, of a 8099, 46455, 54414, 53763, 67076, 67102, 44181, 67084FL, or 67084alt polypeptide for 8099, 46455, 54414, 53763, 67076, 67102, 44181, 67084FL, or 67084alt polypeptide agonist or antagonist activity. In one embodiment, a variegated library of 8099, 46455, 54414, 53763, 67076, 67102, 44181, 67084FL, or 67084alt variants is generated by combinatorial mutagenesis at the nucleic acid level and is encoded by a variegated gene library. A variegated library of 8099, 46455, 54414, 53763, 67076, 67102, 44181, 67084FL, or 67084alt variants can be produced by, for example, enzymatically ligating a mixture of synthetic oligonucleotides into gene sequences such that a degenerate set of potential 8099, 46455, 54414, 53763, 67076, 67102, 44181, 67084FL, or 67084alt sequences is expressible as individual polypeptides, or alternatively, as a set of larger fusion proteins (e.g., for phage display) containing the set of 8099, 46455, 54414, 53763, 67076, 67102, 44181, 67084FL, or 67084alt sequences therein. There are a variety of methods which can be used to produce libraries of potential 8099, 46455, 54414, 53763, 67076, 67102, 44181, 67084FL, or 67084alt variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 8099, 46455, 54414, 53763, 67076, 67102, 44181, 67084FL, or 67084alt sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a 8099, 46455, 54414, 53763, 67076, 67102, 44181, 67084FL, or 67084alt polypeptide coding sequence can be used to generate a variegated population of 8099, 46455, 54414, 53763, 67076, 67102, 44181, 67084FL, or 67084alt fragments for screening and subsequent selection of variants of a 8099, 46455, 54414, 53763, 67076, 67102, 44181, 67084FL, or 67084alt polypeptide. In one embodiment, a library of coding sequence fragments can be generated by treating a double stranded PCR fragment of a 8099, 46455, 54414, 53763, 67076, 67102, 44181, 67084FL, or 67084alt coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 8099, 46455, 54414, 53763, 67076, 67102, 44181, 67084FL, or 67084alt polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 8099, 46455, 54414, 53763, 67076, 67102, 44181, 67084FL, or 67084alt polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 8099, 46455, 54414, 53763, 67076, 67102, 44181, 67084FL, or 67084alt variants (Arkin and Youvan (1992) Proc. Natl. Acad. Sci. USA 89:7811-7815; Delagrave et al. (1993) Protein Engineering 6(3):327-331).

In one embodiment, cell based assays can be exploited to analyze a variegated 8099, 46455, 54414, 53763, 67076, 67102, 44181, 67084FL, or 67084alt library. For example, a library of expression vectors can be transfected into a cell line, which ordinarily responds to 8099, 46455, 54414, 53763, 67076, 67102, 44181, 67084FL, or 67084alt in a particular 8099, 46455, 54414, 53763, 67076, 67102, 44181, 67084FL, or 67084alt substrate-dependent manner. The transfected cells are then contacted with 8099, 46455, 54414, 53763, 67076, 67102, 44181, 67084FL, or 67084alt and the effect of the expression of the mutant on signaling by the 8099, 46455, 54414, 53763, 67076, 67102, 44181, 67084FL, or 67084alt substrate can be detected, e.g., phospholipid transport (e.g., by measuring phospholipid levels inside the cell or its various cellular compartments, within various cellular membranes, or in the extra-cellular medium), hydrolysis of ATP, phosphorylation or dephosphorylation of the HEAT protein, and/or gene transcription. Plasmid DNA can then be recovered from the cells which score for inhibition, or alternatively, potentiation of signaling by the HEAT substrate, or which score for increased or decreased levels of phospholipid transport or ATP hydrolysis, and the individual clones further characterized.

An isolated 8099, 46455, 54414, 53763, 67076, 67102, 44181, 67084FL, or 67084alt polypeptide, or a portion or fragment thereof, can be used as an immunogen to generate antibodies that bind 8099, 46455, 54414, 53763, 67076, 67102, 44181, 67084FL, or 67084alt using standard techniques for polyclonal and monoclonal antibody preparation. A full-length 8099, 46455, 54414, 53763, 67076, 67102, 44181, 67084FL, or 67084alt polypeptide can be used or, alternatively, the invention provides antigenic peptide fragments of 8099, 46455, 54414, 53763, 67076, 67102, 44181, 67084FL, or 67084alt for use as immunogens. The antigenic peptide of 8099, 46455, 54414, 53763, 67076, 67102, 44181, 67084FL, or 67084alt comprises at least 8 amino acid residues of the amino acid sequence shown in SEQ ID NO:47, 50, 53, 56, 59, 62, 65, 68, or 71 and encompasses an epitope of 8099, 46455, 54414, 53763, 67076, 67102, 44181, 67084FL, or 67084alt such that an antibody raised against the peptide forms a specific immune complex with 8099, 46455, 54414, 53763, 67076, 67102, 44181, 67084FL, or 67084alt. Preferably, the antigenic peptide comprises at least 10 amino acid residues, more preferably at least 15 amino acid residues, even more preferably at least 20 amino acid residues, and most preferably at least 30 amino acid residues.

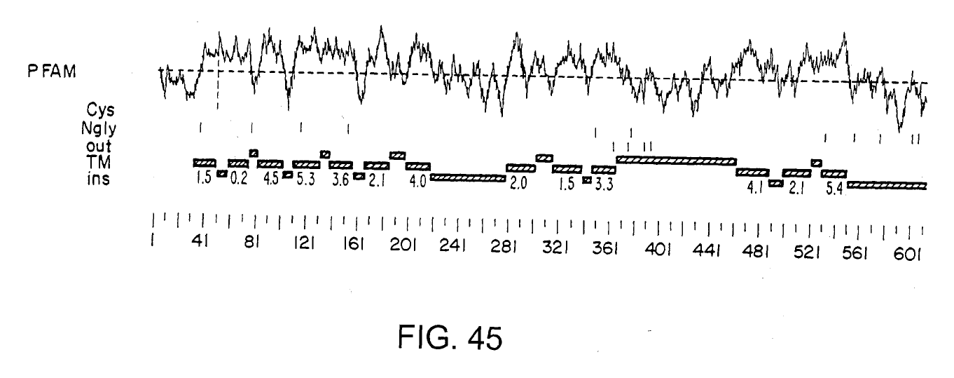

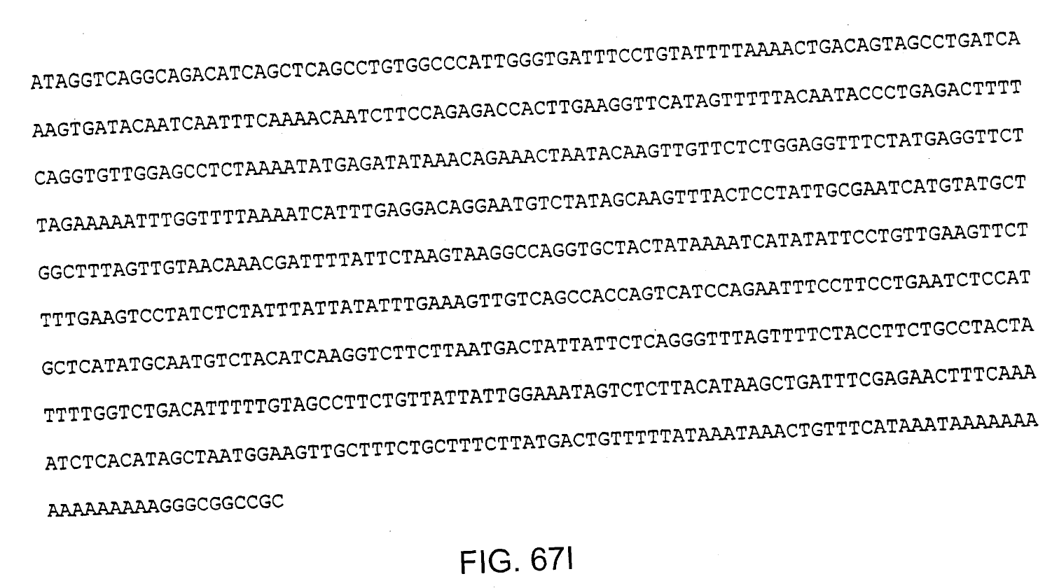

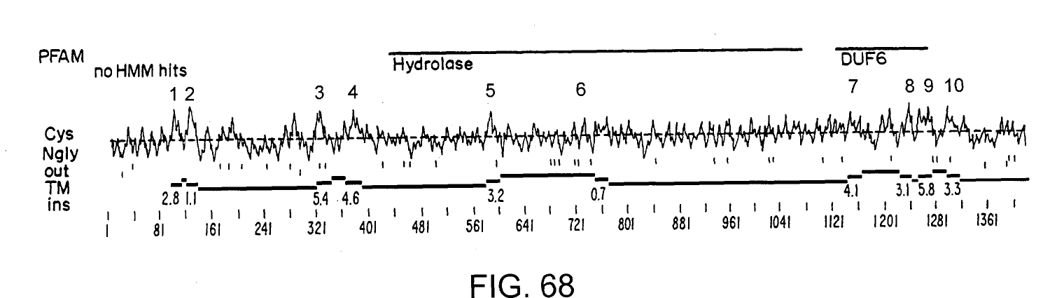

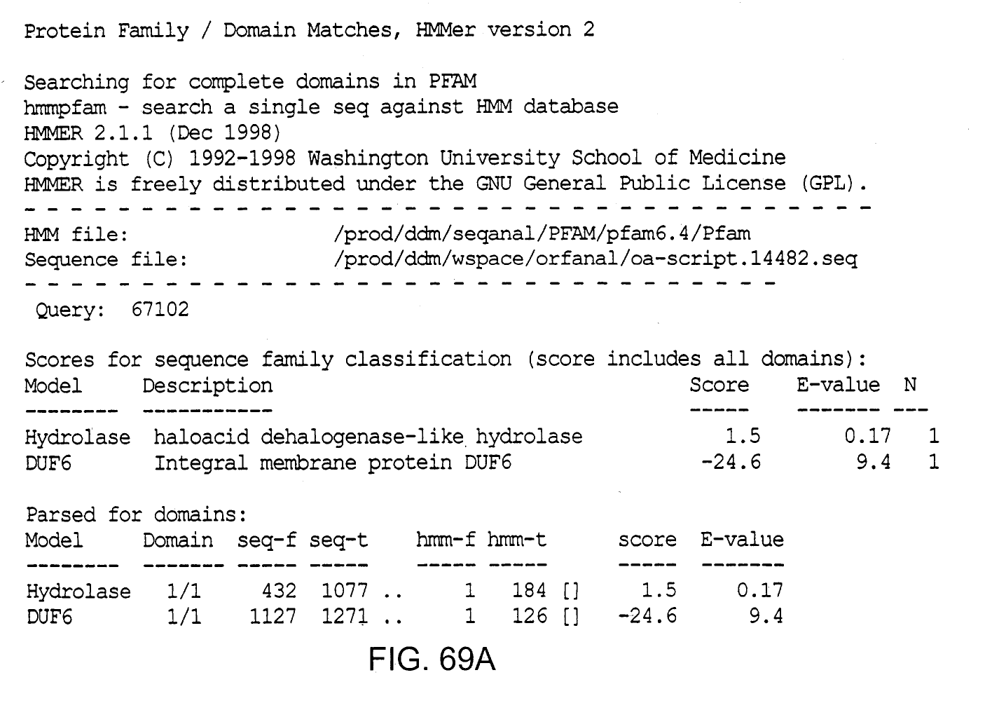

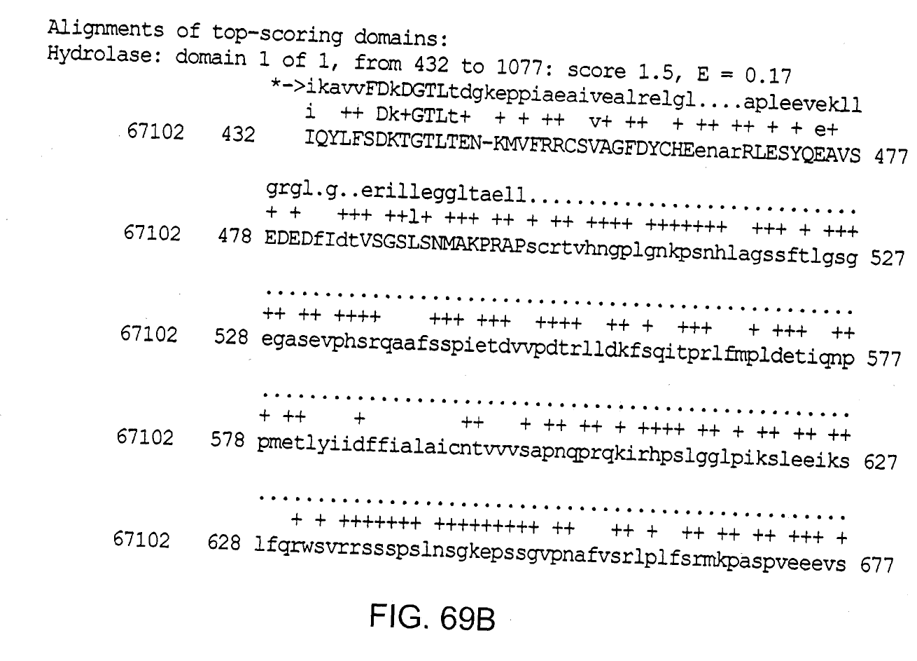

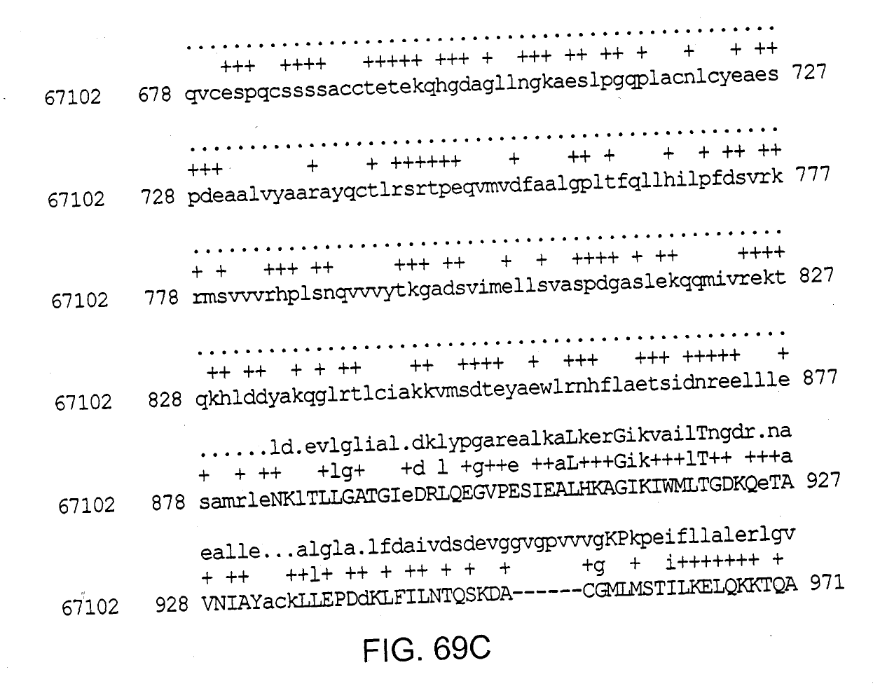

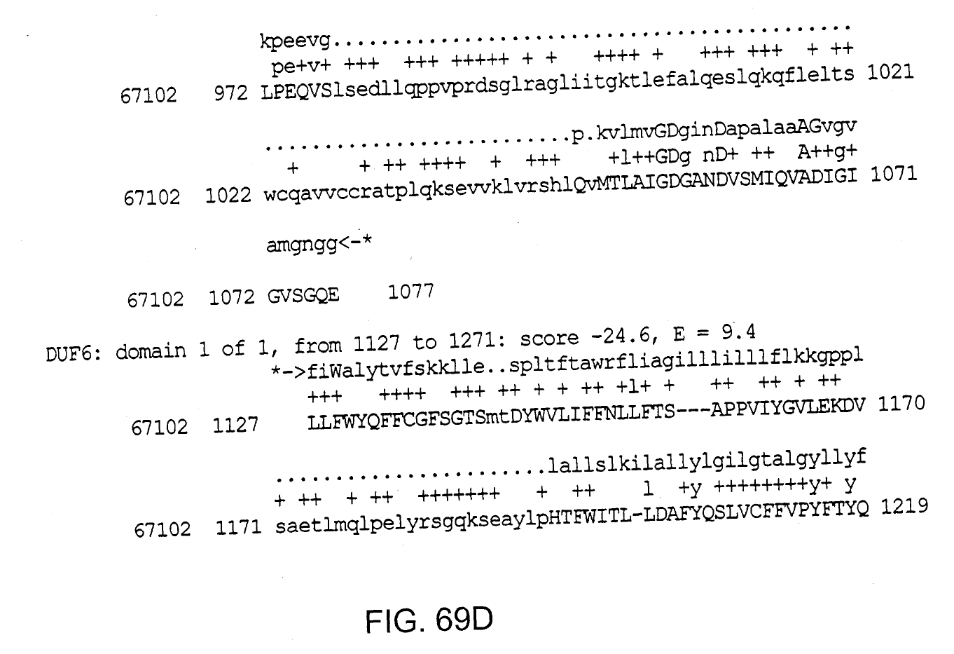

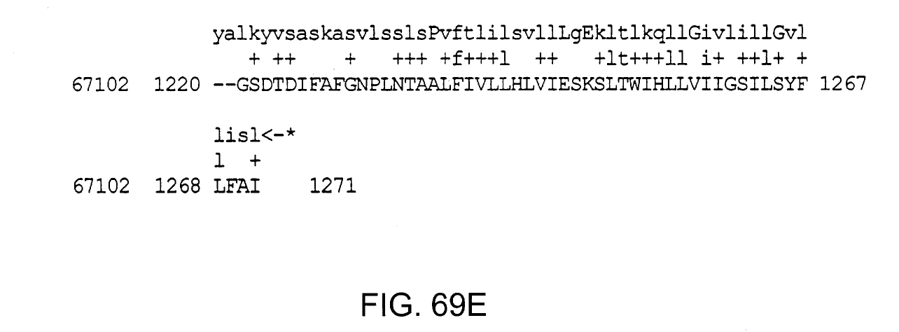

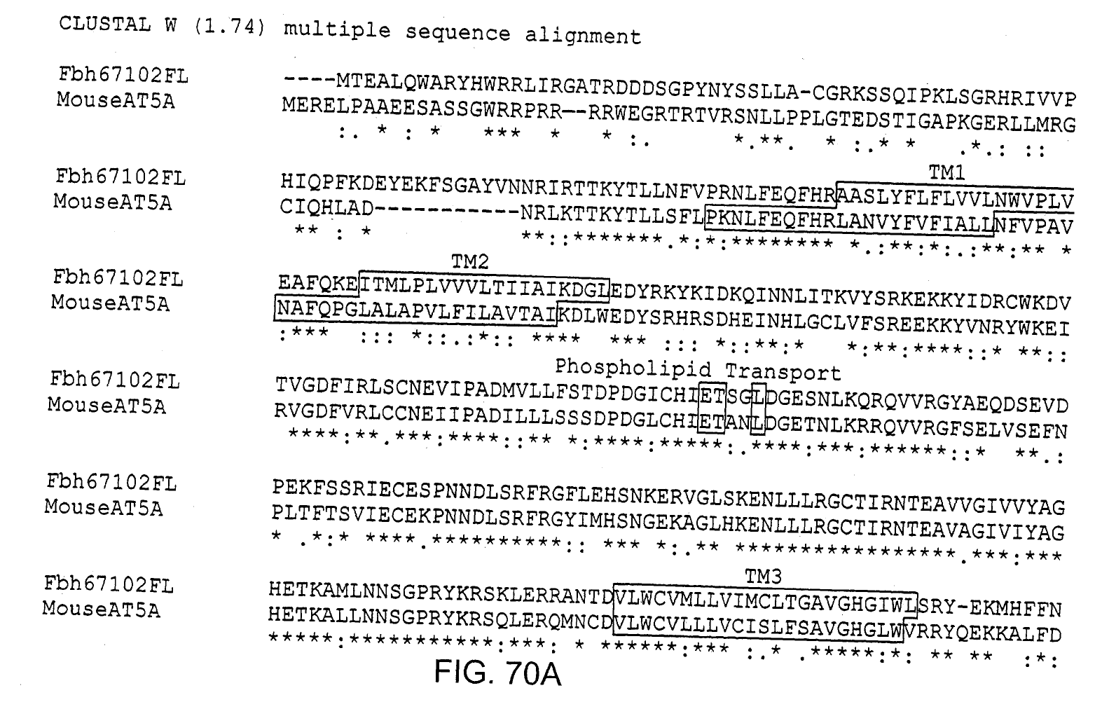

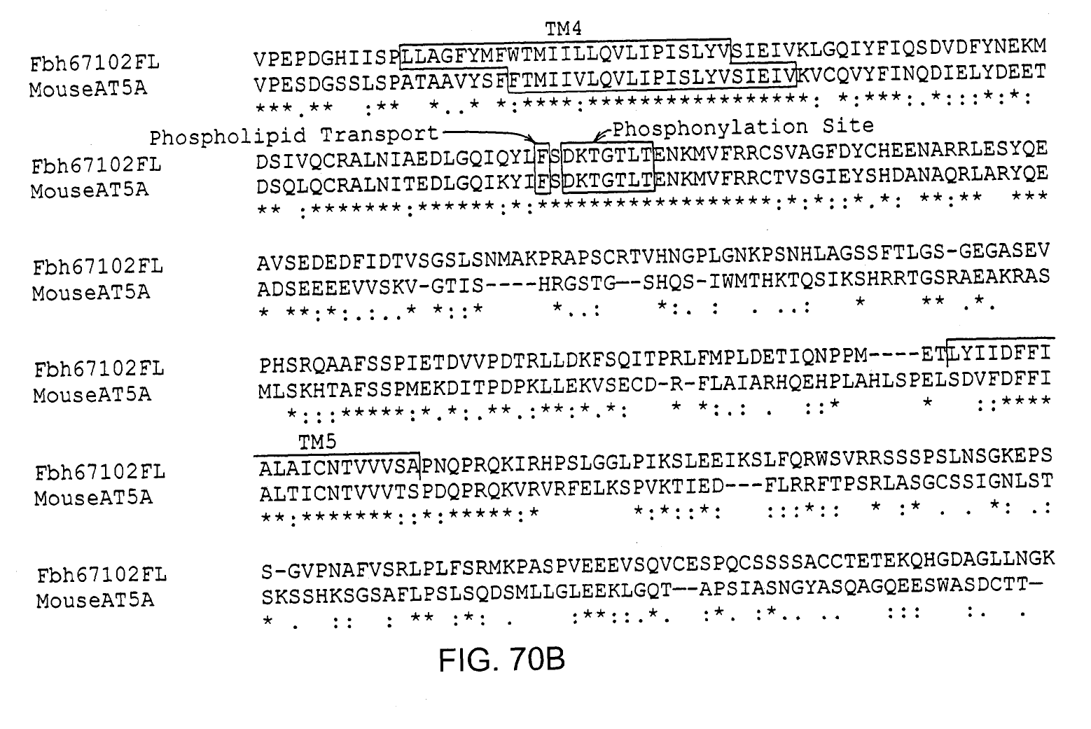

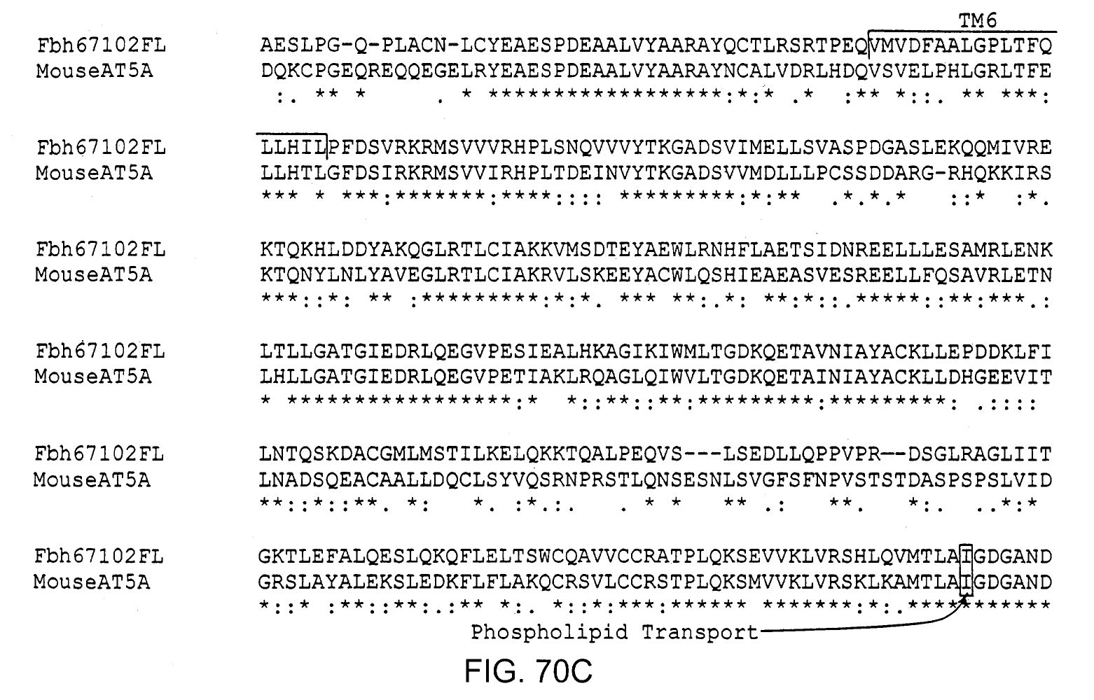

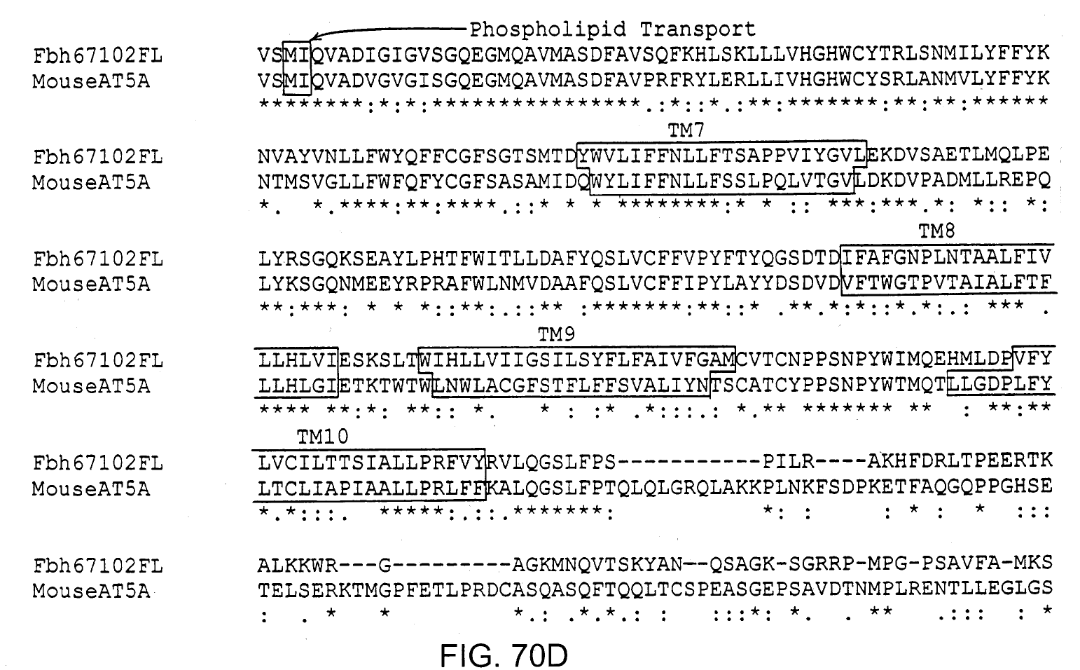

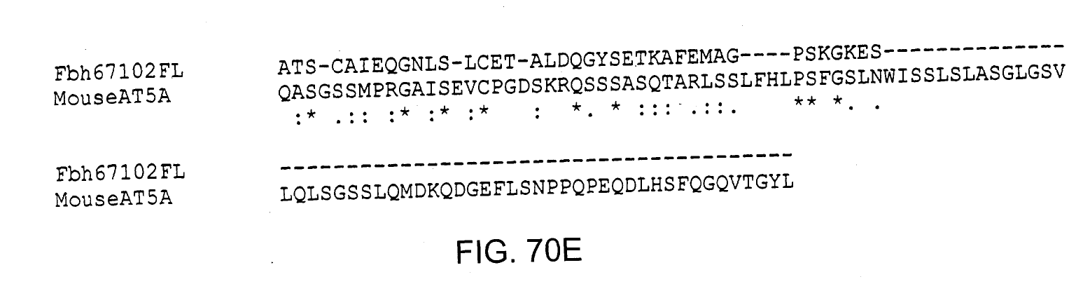

Preferred epitopes encompassed by the antigenic peptide are regions of 8099, 46455, 54414, 53763, 67076, 67102, 44181, 67084FL, or 67084alt that are located on the surface of the polypeptide, e.g., hydrophilic regions, as well as regions with high antigenicity (see, for example, FIGS. 45, 52, 56, 60, 64, 68, 72, 76, and 80).

A 8099, 46455, 54414, 53763, 67076, 67102, 44181, 67084FL, or 67084alt immunogen typically is used to prepare antibodies by immunizing a suitable subject, (e.g., rabbit, goat, mouse or other mammal) with the immunogen. An appropriate immunogenic preparation can contain, for example, recombinantly expressed 8099, 46455, 54414, 53763, 67076, 67102, 44181, 67084FL, or 67084alt polypeptide or a chemically synthesized 8099, 46455, 54414, 53763, 67076, 67102, 44181, 67084FL, or 67084alt polypeptide. The preparation can further include an adjuvant, such as Freund's complete or incomplete adjuvant, or similar immunostimulatory agent. Immunization of a suitable subject with an immunogenic 8099, 46455, 54414, 53763, 67076, 67102, 44181, 67084FL, or 67084alt preparation induces a polyclonal anti-8099, 46455, 54414, 53763, 67076, 67102, 44181, 67084FL, or 67084alt antibody response.

Accordingly, another aspect of the invention pertains to polyclonal anti-8099, 46455, 54414, 53763, 67076, 67102, 44181, 67084FL, or 67084alt antibodies. The term “antibody” as used herein refers to immunoglobulin molecules and immunologically active portions of immunoglobulin molecules, i.e., molecules that contain an antigen binding site which specifically binds (immunoreacts with) an antigen, such as 8099, 46455, 54414, 53763, 67076, 67102, 44181, 67084FL, or 67084alt. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 8099, 46455, 54414, 53763, 67076, 67102, 44181, 67084FL, or 67084alt. The term “monoclonal antibody” or “monoclonal antibody composition”, as used herein, refers to a population of antibody molecules that contain only one species of an antigen binding site capable of immunoreacting with a particular epitope of 8099, 46455, 54414, 53763, 67076, 67102, 44181, 67084FL, or 67084alt. A monoclonal antibody composition thus typically displays a single binding affinity for a particular 8099, 46455, 54414, 53763, 67076, 67102, 44181, 67084FL, or 67084alt polypeptide with which it immunoreacts.

Polyclonal anti-8099, 46455, 54414, 53763, 67076, 67102, 44181, 67084FL, or 67084alt antibodies can be prepared as described above by immunizing a suitable subject with a 8099, 46455, 54414, 53763, 67076, 67102, 44181, 67084FL, or 67084alt immunogen. The anti-8099, 46455, 54414, 53763, 67076, 67102, 44181, 67084FL, or 67084alt antibody titer in the immunized subject can be monitored over time by standard techniques, such as with an enzyme linked immunosorbent assay (ELISA) using immobilized 8099, 46455, 54414, 53763, 67076, 67102, 44181, 67084FL, or 67084alt. If desired, the antibody molecules directed against 8099, 46455, 54414, 53763, 67076, 67102, 44181, 67084FL, or 67084alt can be isolated from the mammal (e.g., from the blood) and further purified by well known techniques, such as protein A chromatography to obtain the IgG fraction. At an appropriate time after immunization, e.g., when the anti-8099, 46455, 54414, 53763, 67076, 67102, 44181, 67084FL, or 67084alt antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem .255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a 8099, 46455, 54414, 53763, 67076, 67102, 44181, 67084FL, or 67084alt immunogen as described above, and the culture supernatants of the resulting hybridoma cells are screened to identify a hybridoma producing a monoclonal antibody that binds 8099, 46455, 54414, 53763, 67076, 67102, 44181, 67084FL, or 67084alt.

Any of the many well known protocols used for fusing lymphocytes and immortalized cell lines can be applied for the purpose of generating an anti-8099, 46455, 54414, 53763, 67076, 67102, 44181, 67084FL, or 67084alt monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 8099, 46455, 54414, 53763, 67076, 67102, 44181, 67084FL, or 67084alt, e.g., using a standard ELISA assay.

Alternative to preparing monoclonal antibody-secreting hybridomas, a monoclonal anti-8099, 46455, 54414, 53763, 67076, 67102, 44181, 67084FL, or 67084alt antibody can be identified and isolated by screening a recombinant combinatorial immunoglobulin library (e.g., an antibody phage display library) with 8099, 46455, 54414, 53763, 67076, 67102, 44181, 67084FL, or 67084alt to thereby isolate immunoglobulin library members that bind 8099, 46455, 54414, 53763, 67076, 67102, 44181, 67084FL, or 67084alt. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246.1275-1281; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-8099, 46455, 54414, 53763, 67076, 67102, 44181, 67084FL, or 67084alt antibodies, such as chimeric and humanized monoclonal antibodies, comprising both human and non-human portions, which can be made using standard recombinant DNA techniques, are within the scope of the invention Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-8099, 46455, 54414, 53763, 67076, 67102, 44181, 67084FL, or 67084alt antibody (e.g., monoclonal antibody) can be used to isolate 8099, 46455, 54414, 53763, 67076, 67102, 44181, 67084FL, or 67084alt by standard techniques, such as affinity chromatography or immunoprecipitation. An anti-8099, 46455, 54414, 53763, 67076, 67102, 44181, 67084FL, or 67084alt antibody can facilitate the purification of natural 8099, 46455, 54414, 53763, 67076, 67102, 44181, 67084FL, or 67084alt from cells and of recombinantly produced 8099, 46455, 54414, 53763, 67076, 67102, 44181, 67084FL, or 67084alt expressed in host cells. Moreover, an anti-8099, 46455, 54414, 53763, 67076, 67102, 44181, 67084FL, or 67084alt antibody can be used to detect 8099, 46455, 54414, 53763, 67076, 67102, 44181, 67084FL, or 67084alt polypeptides (e.g., in a cellular lysate or cell supernatant) in order to evaluate the abundance and pattern of expression of the 8099, 46455, 54414, 53763, 67076, 67102, 44181, 67084FL, or 67084alt polypeptide. Anti-8099, 46455, 54414, 53763, 67076, 67102, 44181, 67084FL, or 67084alt antibodies can be used diagnostically to monitor polypeptide levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, for example recombinant expression vectors, containing a nucleic acid containing a 8099, 46455, 54414, 53763, 67076, 67102, 44181, 67084FL, or 67084alt nucleic acid molecule or vectors containing a nucleic acid molecule which encodes a 8099, 46455, 54414, 53763, 67076, 67102, 44181, 67084FL, or 67084alt polypeptide (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, and the like. The expression vectors of the invention can be introduced into host cells to thereby produce proteins or peptides, including fusion proteins or peptides, encoded by nucleic acids as described herein (e.g., 8099, 46455, 54414, 53763, 67076, 67102, 44181, 67084FL, or 67084alt polypeptides, mutant forms of 8099, 46455, 54414, 53763, 67076, 67102, 44181, 67084FL, or 67084alt polypeptides, fusion proteins, and the like).

Accordingly, an exemplary embodiment provides a method for producing a polypeptide, preferably a 8099, 46455, 54414, 53763, 67076, 67102, 44181, 67084FL, or 67084alt polypeptide, by culturing in a suitable medium a host cell of the invention (e.g., a mammalian host cell such as a non-human mammalian cell) containing a recombinant expression vector, such that the polypeptide is produced.

The recombinant expression vectors of the invention can be designed for expression of 8099, 46455, 54414, 53763, 67076, 67102, 44181, 67084FL, or 67084alt polypeptides in prokaryotic or eukaryotic cells. For example, 8099, 46455, 54414, 53763, 67076, 67102, 44181, 67084FL, or 67084alt polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 8099, 46455, 54414, 53763, 67076, 67102, 44181, 67084FL, or 67084alt activity assays, (e.g., direct assays or competitive assays described in detail below), or to generate antibodies specific for 8099, 46455, 54414, 53763, 67076, 67102, 44181, 67084FL, or 67084alt polypeptides, for example. In a preferred embodiment, a 8099, 46455, 54414, 53763, 67076, 67102, 44181, 67084FL, or 67084alt fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the 8099, 46455, 54414, 53763, 67076, 67102, 44181, 67084FL, or 67084alt expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corporation, San Diego, Calif.).

Alternatively, 8099, 46455, 54414, 53763, 67076, 67102, 44181, 67084FL, or 67084alt polypeptides can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 8099, 46455, 54414, 53763, 67076, 67102, 44181, 67084FL, or 67084alt mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a 8099, 46455, 54414, 53763, 67076, 67102, 44181, 67084FL, or 67084alt nucleic acid molecule of the invention is introduced, e.g., a 8099, 46455, 54414, 53763, 67076, 67102, 44181, 67084FL, or 67084alt nucleic acid molecule within a vector (e.g., a recombinant expression vector) or a 8099, 46455, 54414, 53763, 67076, 67102, 44181, 67084FL, or 67084alt nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 8099, 46455, 54414, 53763, 67076, 67102, 44181, 67084FL, or 67084alt polypeptide can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a 8099, 46455, 54414, 53763, 67076, 67102, 44181, 67084FL, or 67084alt polypeptide or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a 8099, 46455, 54414, 53763, 67076, 67102, 44181, 67084FL, or 67084alt polypeptide. Accordingly, the invention further provides methods for producing a 8099, 46455, 54414, 53763, 67076, 67102, 44181, 67084FL, or 67084alt polypeptide using the host cells of the invention. In one embodiment, the method comprises culturing the host cell of the invention (into which a recombinant expression vector encoding a 8099, 46455, 54414, 53763, 67076, 67102, 44181, 67084FL, or 67084alt polypeptide has been introduced) in a suitable medium such that a 8099, 46455, 54414, 53763, 67076, 67102, 44181, 67084FL, or 67084alt polypeptide is produced. In another embodiment, the method further comprises isolating a 8099, 46455, 54414, 53763, 67076, 67102, 44181, 67084FL, or 67084alt polypeptide from the medium or the host cell.

The host cells of the invention can also be used to produce non-human transgenic animals. For example, in one embodiment, a host cell of the invention is a fertilized oocyte or an embryonic stem cell into which 8099, 46455, 54414, 53763, 67076, 67102, 44181, 67084FL, or 67084alt-coding sequences have been introduced. Such host cells can then be used to create non-human transgenic animals in which exogenous 8099, 46455, 54414, 53763, 67076, 67102, 44181, 67084FL, or 67084alt sequences have been introduced into their genome or homologous recombinant animals in which endogenous 8099, 46455, 54414, 53763, 67076, 67102, 44181, 67084FL, or 67084alt sequences have been altered. Such animals are useful for studying the function and/or activity of a 8099, 46455, 54414, 53763, 67076, 67102, 44181, 67084FL, or 67084alt and for identifying and/or evaluating modulators of 8099, 46455, 54414, 53763, 67076, 67102, 44181, 67084FL, or 67084alt activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 8099, 46455, 54414, 53763, 67076, 67102, 44181, 67084FL, or 67084alt gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a 8099, 46455, 54414, 53763, 67076, 67102, 44181, 67084FL, or 67084alt-encoding nucleic acid into the male pronuclei of a fertilized oocyte, e.g., by microinjection, retroviral infection, and allowing the oocyte to develop in a pseudopregnant female foster animal. The 8099, 46455, 54414, 53763, 67076, 67102, 44181, 67084FL, or 67084alt cDNA sequence of SEQ ID NO:46, 49, 52, 55, or 58 can be introduced as a transgene into the genome of a non-human animal. Alternatively, a nonhuman homologue of a human 8099, 46455, 54414, 53763, 67076, 67102, 44181, 67084FL, or 67084alt gene, such as a mouse or rat 8099, 46455, 54414, 53763, 67076, 67102, 44181, 67084FL, or 67084alt gene, can be used as a transgene. Alternatively, a 8099, 46455, 54414, 53763, 67076, 67102, 44181, 67084FL, or 67084alt gene homologue, such as another 8099, 46455, 54414, 53763, 67076, 67102, 44181, 67084FL, or 67084alt family member, can be isolated based on hybridization to the 8099, 46455, 54414, 53763, 67076, 67102, 44181, 67084FL, or 67084alt cDNA sequences of SEQ ID NO:46, 48, 49, 51, 52, 54, 55, 57, 58, 60, 61, 63, 64, 66, 67, 69, 70, or 72, or the DNA insert of the plasmid deposited with ATCC as Accession Number ______, ______, ______, ______, or ______ (described further in subsection I above) and used as a transgene. Intronic sequences and polyadenylation signals can also be included in the transgene to increase the efficiency of expression of the transgene. A tissue-specific regulatory sequence(s) can be operably linked to a 8099, 46455, 54414, 53763, 67076, 67102, 44181, 67084FL, or 67084alt transgene to direct expression of a 8099, 46455, 54414, 53763, 67076, 67102, 44181, 67084FL, or 67084alt polypeptide to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 8099, 46455, 54414, 53763, 67076, 67102, 44181, 67084FL, or 67084alt transgene in its genome and/or expression of 8099, 46455, 54414, 53763, 67076, 67102, 44181, 67084FL, or 67084alt mRNA in tissues or cells of the animals. A transgenic founder animal can then be used to breed additional animals carrying the transgene. Moreover, transgenic animals carrying a transgene encoding a 8099, 46455, 54414, 53763, 67076, 67102, 44181, 67084FL, or 67084alt polypeptide can further be bred to other transgenic animals carrying other transgenes.

To create a homologous recombinant animal, a vector is prepared which contains at least a portion of a 8099, 46455, 54414, 53763, 67076, 67102, 44181, 67084FL, or 67084alt gene into which a deletion, addition or substitution has been introduced to thereby alter, e.g., functionally disrupt, the 8099, 46455, 54414, 53763, 67076, 67102, 44181, 67084FL, or 67084alt gene. The 8099, 46455, 54414, 53763, 67076, 67102, 44181, 67084FL, or 67084alt gene can be a human gene (e.g., the cDNA of SEQ ID NO:48, 51, 54, 57, or 60), but more preferably, is a non-human homologue of a human 8099, 46455, 54414, 53763, 67076, 67102, 44181, 67084FL, or 67084alt gene (e.g., a cDNA isolated by stringent hybridization with the nucleotide sequence of SEQ ID NO:46, 49, 52, 55, or 58). For example, a mouse 8099, 46455, 54414, 53763, 67076, 67102, 44181, 67084FL, or 67084alt gene can be used to construct a homologous recombination nucleic acid molecule, e.g., a vector, suitable for altering an endogenous 8099, 46455, 54414, 53763, 67076, 67102, 44181, 67084FL, or 67084alt gene in the mouse genome. In a preferred embodiment, the homologous recombination nucleic acid molecule is designed such that, upon homologous recombination, the endogenous 8099, 46455, 54414, 53763, 67076, 67102, 44181, 67084FL, or 67084alt gene is functionally disrupted (i.e., no longer encodes a functional protein; also referred to as a “knock out” vector). Alternatively, the homologous recombination nucleic acid molecule can be designed such that, upon homologous recombination, the endogenous 8099, 46455, 54414, 53763, 67076, 67102, 44181, 67084FL, or 67084alt gene is mutated or otherwise altered but still encodes functional polypeptide (e.g., the upstream regulatory region can be altered to thereby alter the expression of the endogenous 8099, 46455, 54414, 53763, 67076, 67102, 44181, 67084FL, or 67084alt polypeptide). In the homologous recombination nucleic acid molecule, the altered portion of the 8099, 46455, 54414, 53763, 67076, 67102, 44181, 67084FL, or 67084alt gene is flanked at its 5′ and 3′ ends by additional nucleic acid sequence of the 8099, 46455, 54414, 53763, 67076, 67102, 44181, 67084FL, or 67084alt gene to allow for homologous recombination to occur between the exogenous 8099, 46455, 54414, 53763, 67076, 67102, 44181, 67084FL, or 67084alt gene carried by the homologous recombination nucleic acid molecule and an endogenous 8099, 46455, 54414, 53763, 67076, 67102, 44181, 67084FL, or 67084alt gene in a cell, e.g., an embryonic stem cell. The additional flanking 8099, 46455, 54414, 53763, 67076, 67102, 44181, 67084FL, or 67084alt nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 8099, 46455, 54414, 53763, 67076, 67102, 44181, 67084FL, or 67084alt gene has homologously recombined with the endogenous 8099, 46455, 54414, 53763, 67076, 67102, 44181, 67084FL, or 67084alt gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The 8099, 46455, 54414, 53763, 67076, 67102, 44181, 67084FL, or 67084alt nucleic acid molecules, fragments of 8099, 46455, 54414, 53763, 67076, 67102, 44181, 67084FL, or 67084alt polypeptides, anti-8099, 46455, 54414, 53763, 67076, 67102, 44181, 67084FL, or 67084alt antibodies, and or 8099, 46455, 54414, 53763, 67076, 67102, 44181, 67084FL, or 67084alt modulators, (also referred to herein as “active compounds”) of the invention can be incorporated into pharmaceutical compositions suitable for administration. Such compositions typically comprise the nucleic acid molecule, polypeptide, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a 8099, 46455, 54414, 53763, 67076, 67102, 44181, 67084FL, or 67084alt polypeptide or an anti-8099, 46455, 54414, 53763, 67076, 67102, 44181, 67084FL, or 67084alt antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g, with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a polypeptide or antibody can include a single treatment or, preferably, can include a series of treatments.

In a preferred example, a subject is treated with antibody or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, antibodies, and modulators described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

As described herein, an 8099 and/or 46455 polypeptide of the invention has one or more of the following activities: (1) bind a monosaccharide, e.g., D-glucose, D-fructose, D-galactose, and/or mannose, (2) transport monosaccharides across a cell membrane, (3) influence insulin and/or glucagon secretion, (4) maintain sugar homeostasis in a cell, (5) function as a neuronal transporter, and (6) mediate trans-epithelial movement in a cell.

As described herein, a 54414 and/or 53763 protein of the invention has one or more of the following activities: (i) interaction with a 54414 or 53763 substrate (e.g., a potassium ion or a cyclic nucleotide); (ii) conductance or transport of a 54414 or 53763 substrate across a cellular membrane; (iii) interaction with a second non-54414 or 53763 protein (e.g., a 54414 or 53763 polypeptide or a non-54414 or 53763 potassium channel subunit); (iv) modulation (e.g., maintenance and/or rectification) of membrane potentials; (v) regulation of target molecule availability or activity; (vi) modulation of intra- or intercellular signaling and/or gene transcription (e.g., either directly or indirectly); (viii) generation of outwardly rectifying currents; (viii) modulation of membrane excitability; (ix) modulation of the release of neurotransmitters; (x) regulation of contractility (e.g., of smooth muscle cells), secretion, and/or synaptic transmission; and/or (xi) modulation of processes which underlie learning and memory.

Preferred activities of 54414 further include at least one of the following activities: (i) interaction with maxi-K potassium channels (i.e., large conductance channels, in particular Slo); (ii) modulation of maxi-K potassium channel activity (e.g., Slo-mediated activities); (iii) generation of intermediate conductance channels; and/or (iv) regulation of contractility (e.g., of smooth muscle cells), secretion, and/or synaptic transmission, in particular, via modulation of Slo.

Preferred activities of 53763 further include at least one of the following activities:

(i) interaction with Shaker (Sh) potassium channels and/or channel subunits; (ii) modulation of Shaker (Sh) potassium channel activity (e.g., termination of prolonged membrane depolarization; (iii) modulation of high voltage activating channel activity and/or inactivating channel activity, and the like.

As described herein, a 67076, 67102, 44181, 67084FL, or 67084alt polypeptide of the invention has one or more of the following activities: (i) interaction with a 67076, 67102, 44181, 67084FL, or 67084alt substrate or target molecule (e.g., a phospholipid, ATP, or a non-67076, 67102, 44181, 67084FL, or 67084alt protein); (ii) transport of a 67076, 67102, 44181, 67084FL, or 67084alt substrate or target molecule (e.g., an aminophospholipid such as phosphatidylserine or phosphatidylethanolamine) from one side of a cellular membrane to the other; (iii) the ability to be phosphorylated or dephosphorylated; (iv) adoption of an E1 conformation or an E2 conformation; (v) conversion of a 67076, 67102, 44181, 67084FL, or 67084alt substrate or target molecule to a product (e.g., hydrolysis of ATP); (vi) interaction with a second non-67076, 67102, 44181, 67084FL, or 67084alt protein; (vii) modulation of substrate or target molecule location (e.g., modulation of phospholipid location within a cell and/or location with respect to a cellular membrane); (viii) maintenance of aminophospholipid gradients; (ix) modulation of intra- or intercellular signaling and/or gene transcription (e.g., either directly or indirectly); and/or (x) modulation of cellular proliferation, growth, differentiation, apoptosis, absorption, or secretion.

The isolated nucleic acid molecules of the invention can be used, for example, to express 8099, 46455, 54414, 53763, 67076, 67102, 44181, 67084FL, or 67084alt polypeptides (e.g., via a recombinant expression vector in a host cell in gene therapy applications), to detect 8099, 46455, 54414, 53763, 67076, 67102, 44181, 67084FL, or 67084alt mRNA (e.g., in a biological sample) or a genetic alteration in a 8099, 46455, 54414, 53763, 67076, 67102, 44181, 67084FL, or 67084alt gene, and to modulate 8099, 46455, 54414, 53763, 67076, 67102, 44181, 67084FL, or 67084alt activity, as described further below. The 8099, 46455, 54414, 53763, 67076, 67102, 44181, 67084FL, or 67084alt polypeptides can be used to treat disorders characterized by insufficient or excessive production of a 8099, 46455, 54414, 53763, 67076, 67102, 44181, 67084FL, or 67084alt substrate or production or transport of 8099, 46455, 54414, 53763, 67076, 67102, 44181, 67084FL, or 67084alt inhibitors, for example, 8099, 46455, 54414, 53763, 67076, 67102, 44181, 67084FL, or 67084alt associated disorders.

As used herein, a “sugar transporter” includes a protein or polypeptide which is involved in transporting a molecule, e.g., a monosaccharide such as D-glucose, D-fructose, D-galactose or mannose, across the plasma membrane of a cell, e.g., a liver cell, fat cell, muscle cell, or blood cell, such as an erythrocyte. Sugar transporters regulate sugar homeostasis in a cell and, typically, have sugar substrate specificity. Examples of sugar transporters include glucose transporters, fructose transporters, and galactose transporters.

As used herein, a “sugar transporter mediated activity” includes an activity which involves a sugar transporter, e.g., a sugar transporter in a liver cell, fat cell, muscle cell, or blood cell, such as an erythrocyte. Sugar transporter mediated activities include the transport of sugars, e.g., D-glucose, D-fructose, D-galactose or mannose, into and out of cells; the stimulation of molecules that regulate glucose homeostasis (e.g., insulin and glucagon), from cells, e.g., pancreatic cells; and the participation in signal transduction pathways associated with sugar metabolism.

As the 8099 and 46455 molecules of the present invention are sugar transporters, they may be useful for developing novel diagnostic and therapeutic agents for sugar transporter associated disorders. As used herein, the terms “sugar transporter associated disorder” and “8099 and 46455 disorder,” used interchangeably herein, includes a disorder, disease, or condition which is characterized by an aberrant, e.g., upregulated or downregulated, sugar transporter mediated activity. Sugar transporter associated disorders typically result in, e.g., upregulated or downregulated, sugar levels in a cell. Examples of sugar transporter associated disorders include disorders associated with sugar homeostasis, such as obesity, anorexia, type-1 diabetes, type-2 diabetes, hypoglycemia, glycogen storage disease (Von Gierke disease), type I glycogenosis, bipolar disorder, seasonal affective disorder, and cluster B personality disorders.

As used interchangeably herein, a “potassium channel associated disorder” or a “54414 or 53763 associated disorder” include a disorder, disease or condition which is caused or characterized by a misregulation (e.g., downregulation or upregulation) of 54414 or 53763 activity. 54414 or 53763 associated disorders can detrimentally affect cellular functions such as cellular proliferation, growth, differentiation, inter- or intra-cellular communication; tissue function, such as cardiac function or musculoskeletal function; systemic responses in an organism, such as nervous system responses, hormonal responses (e.g., insulin response), or immune responses; and protection of cells from toxic compounds (e.g., carcinogens, toxins, or mutagens).

In a preferred embodiment, 54414 or 53763 associated disorders include CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, seizure disorders, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

54414 or 53763 associated disorders also include cellular proliferation, growth, differentiation, or apoptosis disorders. Cellular proliferation, growth, differentiation, or apoptosis disorders include those disorders that affect cell proliferation, growth, differentiation, or apoptosis processes. As used herein, a “cellular proliferation, growth, differentiation, or apoptosis process” is a process by which a cell increases in number, size or content, by which a cell develops a specialized set of characteristics which differ from that of other cells, or by which a cell undergoes programmed cell death. The 54414 or 53763 molecules of the present invention may modulate cellular growth, proliferation, differentiation, or apoptosis, and may play a role in disorders characterized by aberrantly regulated growth, proliferation, differentiation, or apoptosis. Such disorders include cancer, e.g., carcinoma, sarcoma, or leukemia; tumor angiogenesis and metastasis; skeletal dysplasia; hepatic disorders; and hematopoietic and/or myeloproliferative disorders.

Further examples of 54414 or 53763 associated disorders include cardiac-related disorders. Cardiovascular system disorders in which the 54414 or 53763 molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia. 54414 or 53763 associated disorders also include disorders of the musculoskeletal system such as paralysis and muscle weakness, e.g., ataxia, myotonia, and myokymia.

54414 or 53763 associated or related disorders also include hormonal disorders, such as conditions or diseases in which the production and/or regulation of hormones in an organism is aberrant. Examples of such disorders and diseases include type I and type II diabetes mellitus, pituitary disorders (e.g., growth disorders), thyroid disorders (e.g., hypothyroidism or hyperthyroidism), and reproductive or fertility disorders (e.g., disorders which affect the organs of the reproductive system, e.g., the prostate gland, the uterus, or the vagina; disorders which involve an imbalance in the levels of a reproductive hormone in a subject; disorders affecting the ability of a subject to reproduce; and disorders affecting secondary sex characteristic development, e.g., adrenal hyperplasia).

54414 or 53763 associated or related disorders also include immune disorders, such as autoimmune disorders or immune deficiency disorders, e.g., congenital X-linked infantile hypogammaglobulinemia, transient hypogammaglobulinemia, common variable immunodeficiency, selective IgA deficiency, chronic mucocutaneous candidiasis, or severe combined immunodeficiency.

As used interchangeably herein, a “phospholipid transporter associated disorder” or a “67076, 67102, 44181, 67084FL, or 67084alt associated disorder” includes a disorder, disease or condition which is caused or characterized by a misregulation (e.g., downregulation or upregulation) of 67076, 67102, 44181, 67084FL, or 67084alt activity. 67076, 67102, 44181, 67084FL, or 67084alt associated disorders can detrimentally affect cellular functions such as cellular proliferation, growth, differentiation, inter- or intra-cellular communication; tissue function, such as cardiac function or musculoskeletal function; systemic responses in an organism, such as nervous system responses, hormonal responses (e.g., insulin response), or immune responses; and protection of cells from toxic compounds (e.g., carcinogens, toxins, or mutagens). Examples of 67076, 67102, 44181, 67084FL, or 67084alt associated disorders include CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, seizure disorders, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further examples of 67076, 67102, 44181, 67084FL, or 67084alt associated disorders include cardiac-related disorders. Cardiovascular system disorders in which the 67076, 67102, 44181, 67084FL, or 67084alt molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia. 67076, 67102, 44181, 67084FL, or 67084alt associated disorders also include disorders of the musculoskeletal system such as paralysis and muscle weakness, e.g., ataxia, myotonia, and myokymia.

67076, 67102, 44181, 67084FL, or 67084alt associated disorders also include cellular proliferation, growth, or differentiation disorders. Cellular proliferation, growth, or differentiation disorders include those disorders that affect cell proliferation, growth, or differentiation processes. As used herein, a “cellular proliferation, growth, or differentiation process” is a process by which a cell increases in number, size or content, or by which a cell develops a specialized set of characteristics which differ from that of other cells. The 67076, 67102, 44181, 67084FL, or 67084alt molecules of the present invention are involved in phospholipid transport mechanisms, which are known to be involved in cellular growth, proliferation, and differentiation processes. Thus, the 67076, 67102, 44181, 67084FL, or 67084alt molecules may modulate cellular growth, proliferation, or differentiation, and may play a role in disorders characterized by aberrantly regulated growth, proliferation, or differentiation. Such disorders include cancer, e.g., carcinoma, sarcoma, or leukemia; tumor angiogenesis and metastasis; skeletal dysplasia; hepatic disorders; and hematopoietic and/or myeloproliferative disorders.

67076, 67102, 44181, 67084FL, or 67084alt associated or related disorders also include hormonal disorders, such as conditions or diseases in which the production and/or regulation of hormones in an organism is aberrant. Examples of such disorders and diseases include type I and type II diabetes mellitus, pituitary disorders (e.g., growth disorders), thyroid disorders (e.g., hypothyroidism or hyperthyroidism), and reproductive or fertility disorders (e.g., disorders which affect the organs of the reproductive system, e.g., the prostate gland, the uterus, or the vagina; disorders which involve an imbalance in the levels of a reproductive hormone in a subject; disorders affecting the ability of a subject to reproduce; and disorders affecting secondary sex characteristic development, e.g., adrenal hyperplasia).

67076, 67102, 44181, 67084FL, or 67084alt associated or related disorders also include immune disorders, such as autoimmune disorders or immune deficiency disorders, e.g., congenital X-linked infantile hypogammaglobulinemia, transient hypogammaglobulinemia, common variable immunodeficiency, selective IgA deficiency, chronic mucocutaneous candidiasis, or severe combined immunodeficiency.

8099, 46455, 54414, 53763, 67076, 67102, 44181, 67084FL, or 67084alt associated or related disorders also include disorders affecting tissues in which 8099, 46455, 54414, 53763, 67076, 67102, 44181, 67084FL, or 67084alt protein is expressed.

In addition, the 8099, 46455, 54414, 53763, 67076, 67102, 44181, 67084FL, or 67084alt polypeptides can be used to screen for naturally occurring 8099, 46455, 54414, 53763, 67076, 67102, 44181, 67084FL, or 67084alt substrates, to screen for drugs or compounds which modulate 8099, 46455, 54414, 53763, 67076, 67102, 44181, 67084FL, or 67084alt activity, as well as to treat disorders characterized by insufficient or excessive production of 8099, 46455, 54414, 53763, 67076, 67102, 44181, 67084FL, or 67084alt polypeptide or production of 8099, 46455, 54414, 53763, 67076, 67102, 44181, 67084FL, or 67084alt polypeptide forms which have decreased, aberrant or unwanted activity compared to 8099, 46455, 54414, 53763, 67076, 67102, 44181, 67084FL, or 67084alt wild type polypeptide (e.g., sugar transporter associated disorder, potassium channel associated disorders, a phospholipid transporter-associated disorders). Moreover, the anti-8099, 46455, 54414, 53763, 67076, 67102, 44181, 67084FL, or 67084alt antibodies of the invention can be used to detect and isolate 8099, 46455, 54414, 53763, 67076, 67102, 44181, 67084FL, or 67084alt polypeptides, to regulate the bioavailability of 8099, 46455, 54414, 53763, 67076, 67102, 44181, 67084FL, or 67084alt polypeptides, and modulate 8099, 46455, 54414, 53763, 67076, 67102, 44181, 67084FL, or 67084alt activity.

A. Screening Assays

The invention provides a method (also referred to herein as a “screening assay”) for identifying modulators, i e., candidate or test compounds or agents (e.g., peptides, peptidomimetics, small molecules or other drugs) which bind to 8099, 46455, 54414, 53763, 67076, 67102, 44181, 67084FL, or 67084alt polypeptides, have a stimulatory or inhibitory effect on, for example, 8099, 46455, 54414, 53763, 67076, 67102, 44181, 67084FL, or 67084alt expression or 8099, 46455, 54414, 53763, 67076, 67102, 44181, 67084FL, or 67084alt activity, or have a stimulatory or inhibitory effect on, for example, the expression or activity of a 8099, 46455, 54414, 53763, 67076, 67102, 44181, 67084FL, or 67084alt substrate.

In one embodiment, the invention provides assays for screening candidate or test compounds which are substrates of a 8099, 46455, 54414, 53763, 67076, 67102, 44181, 67084FL, or 67084alt polypeptide or polypeptide or biologically active portion thereof. In another embodiment, the invention provides assays for screening candidate or test compounds which bind to or modulate the activity of a 8099, 46455, 54414, 53763, 67076, 67102, 44181, 67084FL, or 67084alt polypeptide or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 8099, 46455, 54414, 53763, 67076, 67102, 44181, 67084FL, or 67084alt polypeptide or biologically active portion thereof is contacted with a test compound and the ability of the test compound to modulate 8099, 46455, 54414, 53763, 67076, 67102, 44181, 67084FL, or 67084alt activity is determined.

Determining the ability of the test compound to modulate 8099 or 46455 activity can be accomplished by monitoring, for example, intracellular or extracellular D-glucose, D-fructose, D-galactose, and/or mannose concentration, or insulin or glucagon secretion. The cell, for example, can be of mammalian origin, e.g., a liver cell, fat cell, muscle cell, or a blood cell, such as an erythrocyte.

Determining the ability of the test compound to modulate 54414 or 53763 activity can be accomplished by monitoring, for example, potassium current, neurotransmitter release, and/or membrane excitability in a cell which expresses 54414 or 53763. The cell, for example, can be of mammalian origin, e.g., a neuronal cell.

Determining the ability of the test compound to modulate 67076, 67102, 44181, 67084FL, or 67084alt activity can be accomplished by monitoring, for example, (i) interaction of 67076, 67102, 44181, 67084FL, or 67084alt with a 67076, 67102, 44181, 67084FL, or 67084alt substrate or target molecule (e.g., a phospholipid, ATP, or a non-67076, 67102, 44181, 67084FL, or 67084alt protein); (ii) transport of a 67076, 67102, 44181, 67084FL, or 67084alt substrate or target molecule (e.g., an aminophospholipid such as phosphatidylserine or phosphatidylethanolamine) from one side of a cellular membrane to the other; (iii) the ability of 67076, 67102, 44181, 67084FL, or 67084alt to be phosphorylated or dephosphorylated; (iv) adoption by 67076, 67102, 44181, 67084FL, or 67084alt of an E1 conformation or an E2 conformation; (v) conversion of a 67076, 67102, 44181, 67084FL, or 67084alt substrate or target molecule to a product (e.g., hydrolysis of ATP); (vi) interaction of 67076, 67102, 44181, 67084FL, or 67084alt with a second non-67076, 67102, 44181, 67084FL, or 67084alt protein; (vii) modulation of substrate or target molecule location (e.g., modulation of phospholipid location within a cell and/or location with respect to a cellular membrane); (viii) maintenance of aminophospholipid gradients; (ix) modulation of intra- or intercellular signaling and/or gene transcription (e.g., either directly or indirectly); and/or (x) modulation of cellular proliferation, growth, differentiation, apoptosis, absorption, and/or secretion.

The ability of the test compound to modulate 8099, 46455, 54414, 53763, 67076, 67102, 44181, 67084FL, or 67084alt binding to a substrate or to bind to 8099, 46455, 54414, 53763, 67076, 67102, 44181, 67084FL, or 67084alt can also be determined. Determining the ability of the test compound to modulate 8099, 46455, 54414, 53763, 67076, 67102, 44181, 67084FL, or 67084alt binding to a substrate can be accomplished, for example, by coupling the 8099, 46455, 54414, 53763, 67076, 67102, 44181, 67084FL, or 67084alt substrate with a radioisotope or enzymatic label such that binding of the 8099, 46455, 54414, 53763, 67076, 67102, 44181, 67084FL, or 67084alt substrate to 8099, 46455, 54414, 53763, 67076, 67102, 44181, 67084FL, or 67084alt can be determined by detecting the labeled 8099, 46455, 54414, 53763, 67076, 67102, 44181, 67084FL, or 67084alt substrate in a complex. Alternatively, 8099, 46455, 54414, 53763, 67076, 67102, 44181, 67084FL, or 67084alt could be coupled with a radioisotope or enzymatic label to monitor the ability of a test compound to modulate 8099, 46455, 54414, 53763, 67076, 67102, 44181, 67084FL, or 67084alt binding to a 8099, 46455, 54414, 53763, 67076, 67102, 44181, 67084FL, or 67084alt substrate in a complex. Determining the ability of the test compound to bind 8099, 46455, 54414, 53763, 67076, 67102, 44181, 67084FL, or 67084alt can be accomplished, for example, by coupling the 8099, 46455, 54414, 53763, 67076, 67102, 44181, 67084FL, or 67084alt substrate with a radioisotope or enzymatic label such that binding of the 8099, 46455, 54414, 53763, 67076, 67102, 44181, 67084FL, or 67084alt substrate to 8099, 46455, 54414, 53763, 67076, 67102, 44181, 67084FL, or 67084alt can be determined by detecting the labeled 8099, 46455, 54414, 53763, 67076, 67102, 44181, 67084FL, or 67084alt substrate in a complex. Alternatively, 8099, 46455, 54414, 53763, 67076, 67102, 44181, 67084FL, or 67084alt could be coupled with a radioisotope or enzymatic label to monitor the ability of a test compound to modulate 8099, 46455, 54414, 53763, 67076, 67102, 44181, 67084FL, or 67084alt binding to a 8099, 46455, 54414, 53763, 67076, 67102, 44181, 67084FL, or 67084alt substrate in a complex. Determining the ability of the test compound to bind 8099, 46455, 54414, 53763, 67076, 67102, 44181, 67084FL, or 67084alt can be accomplished, for example, by coupling the compound with a radioisotope or enzymatic label such that binding of the compound to 8099, 46455, 54414, 53763, 67076, 67102, 44181, 67084FL, or 67084alt can be determined by detecting the labeled 8099, 46455, 54414, 53763, 67076, 67102, 44181, 67084FL, or 67084alt compound in a complex. For example, compounds (e.g., 8099, 46455, 54414, 53763, 67076, 67102, 44181, 67084FL, or 67084alt substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 8099, 46455, 54414, 53763, 67076, 67102, 44181, 67084FL, or 67084alt substrate) to interact with 8099, 46455, 54414, 53763, 67076, 67102, 44181, 67084FL, or 67084alt without the labeling of any of the interactants. For example, a microphysiometer can be used to detect the interaction of a compound with 8099, 46455, 54414, 53763, 67076, 67102, 44181, 67084FL, or 67084alt without the labeling of either the compound or the 8099, 46455, 54414, 53763, 67076, 67102, 44181, 67084FL, or 67084alt. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 8099, 46455, 54414, 53763, 67076, 67102, 44181, 67084FL, or 67084alt.

In another embodiment, an assay is a cell-based assay comprising contacting a cell expressing a 8099, 46455, 54414, 53763, 67076, 67102, 44181, 67084FL, or 67084alt target molecule (e.g., a 8099, 46455, 54414, 53763, 67076, 67102, 44181, 67084FL, or 67084alt substrate) with a test compound and determining the ability of the test compound to modulate (e.g., stimulate or inhibit) the activity of the 8099, 46455, 54414, 53763, 67076, 67102, 44181, 67084FL, or 67084alt target molecule. Determining the ability of the test compound to modulate the activity of a 8099, 46455, 54414, 53763, 67076, 67102, 44181, 67084FL, or 67084alt target molecule can be accomplished, for example, by determining the cellular location of the target molecule, or by determining whether the target molecule (e.g., ATP) has been hydrolyzed.

Determining the ability of the 8099, 46455, 54414, 53763, 67076, 67102, 44181, 67084FL, or 67084alt polypeptide, or a biologically active fragment thereof, to bind to or interact with a 8099, 46455, 54414, 53763, 67076, 67102, 44181, 67084FL, or 67084alt target molecule can be accomplished by one of the methods described above for determining direct binding. In a preferred embodiment, determining the ability of the 8099, 46455, 54414, 53763, 67076, 67102, 44181, 67084FL, or 67084alt polypeptide to bind to or interact with a 8099, 46455, 54414, 53763, 67076, 67102, 44181, 67084FL, or 67084alt target molecule can be accomplished by determining the activity of the target molecule. For example, the activity of the target molecule can be determined by detecting the cellular location of target molecule, detecting catalytic/enzymatic activity of the target molecule upon an appropriate substrate, detecting induction of a metabolite of the target molecule (e.g., detecting the products of ATP hydrolysis, changes in intracellular K⁺ levels) detecting the induction of a reporter gene (comprising a target-responsive regulatory element operatively linked to a nucleic acid encoding a detectable marker, e.g., luciferase), or detecting a target-regulated cellular response (i.e., membrane excitability, or cell growth, proliferation, differentiation, or apoptosis, sugar transport).

In yet another embodiment, an assay of the present invention is a cell-free assay in which a 8099, 46455, 54414, 53763, 67076, 67102, 44181, 67084FL, or 67084alt polypeptide or biologically active portion thereof is contacted with a test compound and the ability of the test compound to bind to the 8099, 46455, 54414, 53763, 67076, 67102, 44181, 67084FL, or 67084alt polypeptide or biologically active portion thereof is determined. Preferred biologically active portions of the 8099, 46455, 54414, 53763, 67076, 67102, 44181, 67084FL, or 67084alt polypeptides to be used in assays of the present invention include fragments which participate in interactions with non-8099, 46455, 54414, 53763, 67076, 67102, 44181, 67084FL, or 67084alt 3 molecules, e.g., fragments with high surface probability scores (see, for example, FIGS. 45, 52, 56, 60, 64, 68, 72, 76, and 80). Binding of the test compound to the 8099, 46455, 54414, 53763, 67076, 67102, 44181, 67084FL, or 67084alt polypeptide can be determined either directly or indirectly as described above. In a preferred embodiment, the assay includes contacting the 8099, 46455, 54414, 53763, 67076, 67102, 44181, 67084FL, or 67084alt polypeptide or biologically active portion thereof with a known compound which binds 8099, 46455, 54414, 53763, 67076, 67102, 44181, 67084FL, or 67084alt to form an assay mixture, contacting the assay mixture with a test compound, and determining the ability of the test compound to interact with a 8099, 46455, 54414, 53763, 67076, 67102, 44181, 67084FL, or 67084alt polypeptide, wherein determining the ability of the test compound to interact with a 8099, 46455, 54414, 53763, 67076, 67102, 44181, 67084FL, or 67084alt polypeptide comprises determining the ability of the test compound to preferentially bind to 8099, 46455, 54414, 53763, 67076, 67102, 44181, 67084FL, or 67084alt or biologically active portion thereof as compared to the known compound.

In another embodiment, the assay is a cell-free assay in which a 8099, 46455, 54414, 53763, 67076, 67102, 44181, 67084FL, or 67084alt polypeptide or biologically active portion thereof is contacted with a test compound and the ability of the test compound to modulate (e.g., stimulate or inhibit) the activity of the 8099, 46455, 54414, 53763, 67076, 67102, 44181, 67084FL, or 67084alt polypeptide or biologically active portion thereof is determined. Determining the ability of the test compound to modulate the activity of a 8099, 46455, 54414, 53763, 67076, 67102, 44181, 67084FL, or 67084alt polypeptide can be accomplished, for example, by determining the ability of the 8099, 46455, 54414, 53763, 67076, 67102, 44181, 67084FL, or 67084alt polypeptide to bind to a 8099, 46455, 54414, 53763, 67076, 67102, 44181, 67084FL, or 67084alt target molecule by one of the methods described above for determining direct binding. Determining the ability of the 8099, 46455, 54414, 53763, 67076, 67102, 44181, 67084FL, or 67084alt polypeptide to bind to a 8099, 46455, 54414, 53763, 67076, 67102, 44181, 67084FL, or 67084alt target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a 8099, 46455, 54414, 53763, 67076, 67102, 44181, 67084FL, or 67084alt polypeptide can be accomplished by determining the ability of the 8099, 46455, 54414, 53763, 67076, 67102, 44181, 67084FL, or 67084alt polypeptide to further modulate the activity of a downstream effector of a 8099, 46455, 54414, 53763, 67076, 67102, 44181, 67084FL, or 67084alt target molecule. For example, the activity of the effector molecule on an appropriate target can be determined or the binding of the effector to an appropriate target can be determined as previously described.

In yet another embodiment, the cell-free assay involves contacting a 8099, 46455, 54414, 53763, 67076, 67102, 44181, 67084FL, or 67084alt polypeptide or biologically active portion thereof with a known compound which binds the 8099, 46455, 54414, 53763, 67076, 67102, 44181, 67084FL, or 67084alt polypeptide to form an assay mixture, contacting the assay mixture with a test compound, and determining the ability of the test compound to interact with the 8099, 46455, 54414, 53763, 67076, 67102, 44181, 67084FL, or 67084alt polypeptide, wherein determining the ability of the test compound to interact with the 8099, 46455, 54414, 53763, 67076, 67102, 44181, 67084FL, or 67084alt polypeptide comprises determining the ability of the 8099, 46455, 54414, 53763, 67076, 67102, 44181, 67084FL, or 67084alt polypeptide to preferentially bind to or modulate the activity of a 8099, 46455, 54414, 53763, 67076, 67102, 44181, 67084FL, or 67084alt target molecule.

The cell-free assays of the present invention are amenable to use of both soluble and/or membrane-bound forms of isolated proteins (e.g, 8099, 46455, 54414, 53763, 67076, 67102, 44181, 67084FL, or 67084alt proteins or biologically active portions thereof). In the case of cell-free assays in which a membrane-bound form of an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either 8099, 46455, 54414, 53763, 67076, 67102, 44181, 67084FL, or 67084alt or its target molecule to facilitate separation of complexed from uncomplexed forms of one or both of the proteins, as well as to accommodate automation of the assay. Binding of a test compound to a 8099, 46455, 54414, 53763, 67076, 67102, 44181, 67084FL, or 67084alt polypeptide, or interaction of a 8099, 46455, 54414, 53763, 67076, 67102, 44181, 67084FL, or 67084alt polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/8099, 46455, 54414, 53763, 67076, 67102, 44181, 67084FL, or 67084alt fusion proteins or glutathione-S-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or 8099, 46455, 54414, 53763, 67076, 67102, 44181, 67084FL, or 67084alt polypeptide, and the mixture incubated under conditions conducive to complex formation (e.g, at physiological conditions for salt and pH). Following incubation, the beads or micrometer plate wells are washed to remove any unbound components, the matrix immobilized in the case of beads, complex determined either directly or indirectly, for example, as described above. Alternatively, the complexes can be dissociated from the matrix, and the level of 8099, 46455, 54414, 53763, 67076, 67102, 44181, 67084FL, or 67084alt binding or activity determined using standard techniques.

Other techniques for immobilizing proteins on matrices can also be used in the screening assays of the invention. For example, either a 8099, 46455, 54414, 53763, 67076, 67102, 44181, 67084FL, or 67084alt polypeptide or a 8099, 46455, 54414, 53763, 67076, 67102, 44181, 67084FL, or 67084alt target molecule can be immobilized utilizing conjugation of biotin and streptavidin. Biotinylated 8099, 46455, 54414, 53763, 67076, 67102, 44181, 67084FL, or 67084alt polypeptide, substrate, or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 8099, 46455, 54414, 53763, 67076, 67102, 44181, 67084FL, or 67084alt polypeptide or target molecules but which do not interfere with binding of the 8099, 46455, 54414, 53763, 67076, 67102, 44181, 67084FL, or 67084alt polypeptide to its target molecule can be derivatized to the wells of the plate, and unbound target or 8099, 46455, 54414, 53763, 67076, 67102, 44181, 67084FL, or 67084alt polypeptide trapped in the wells by antibody conjugation. Methods for detecting such complexes, in addition to those described above for the GST-immobilized complexes, include immunodetection of complexes using antibodies reactive with the 8099, 46455, 54414, 53763, 67076, 67102, 44181, 67084FL, or 67084alt polypeptide or target molecule, as well as enzyme-linked assays which rely on detecting an enzymatic activity associated with the 8099, 46455, 54414, 53763, 67076, 67102, 44181, 67084FL, or 67084alt polypeptide or target molecule.

In another embodiment, modulators of 8099, 46455, 54414, 53763, 67076, 67102, 44181, 67084FL, or 67084alt expression are identified in a method wherein a cell is contacted with a candidate compound and the expression of 8099, 46455, 54414, 53763, 67076, 67102, 44181, 67084FL, or 67084alt mRNA or polypeptide in the cell is determined. The level of expression of 8099, 46455, 54414, 53763, 67076, 67102, 44181, 67084FL, or 67084alt mRNA or polypeptide in the presence of the candidate compound is compared to the level of expression of 8099, 46455, 54414, 53763, 67076, 67102, 44181, 67084FL, or 67084alt mRNA or polypeptide in the absence of the candidate compound. The candidate compound can then be identified as a modulator of 8099, 46455, 54414, 53763, 67076, 67102, 44181, 67084FL, or 67084alt expression based on this comparison. For example, when expression of 8099, 46455, 54414, 53763, 67076, 67102, 44181, 67084FL, or 67084alt mRNA or polypeptide is greater (statistically significantly greater) in the presence of the candidate compound than in its absence, the candidate compound is identified as a stimulator of 8099, 46455, 54414, 53763, 67076, 67102, 44181, 67084FL, or 67084alt mRNA or polypeptide expression. Alternatively, when expression of 8099, 46455, 54414, 53763, 67076, 67102, 44181, 67084FL, or 67084alt mRNA or polypeptide is less (statistically significantly less) in the presence of the candidate compound than in its absence, the candidate compound is identified as an inhibitor of 8099, 46455, 54414, 53763, 67076, 67102, 44181, 67084FL, or 67084alt mRNA or polypeptide expression. The level of 8099, 46455, 54414, 53763, 67076, 67102, 44181, 67084FL, or 67084alt mRNA or polypeptide expression in the cells can be determined by methods described herein for detecting 8099, 46455, 54414, 53763, 67076, 67102, 44181, 67084FL, or 67084alt mRNA or polypeptide.

In yet another aspect of the invention, the 8099, 46455, 54414, 53763, 67076, 67102, 44181, 67084FL, or 67084alt polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 8099, 46455, 54414, 53763, 67076, 67102, 44181, 67084FL, or 67084alt (“8099, 46455, 54414, 53763, 67076, 67102, 44181, 67084FL, or 67084alt-binding proteins” or “8099, 46455, 54414, 53763, 67076, 67102, 44181, 67084FL, or 67084alt-bp”) and are involved in 8099, 46455, 54414, 53763, 67076, 67102, 44181, 67084FL, or 67084alt activity. Such 8099, 46455, 54414, 53763, 67076, 67102, 44181, 67084FL, or 67084alt-binding proteins are also likely to be involved in the propagation of signals by the 8099, 46455, 54414, 53763, 67076, 67102, 44181, 67084FL, or 67084alt polypeptides or 8099, 46455, 54414, 53763, 67076, 67102, 44181, 67084FL, or 67084alt targets as, for example, downstream elements of a 8099, 46455, 54414, 53763, 67076, 67102, 44181, 67084FL, or 67084alt-mediated signaling pathway. Alternatively, such 8099, 46455, 54414, 53763, 67076, 67102, 44181, 67084FL, or 67084alt-binding proteins are likely to be 8099, 46455, 54414, 53763, 67076, 67102, 44181, 67084FL, or 67084alt inhibitors.

The two-hybrid system is based on the modular nature of most transcription factors, which consist of separable DNA-binding and activation domains. Briefly, the assay utilizes two different DNA constructs. In one construct, the gene that codes for a 8099, 46455, 54414, 53763, 67076, 67102, 44181, 67084FL, or 67084alt polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 8099, 46455, 54414, 53763, 67076, 67102, 44181, 67084FL, or 67084alt-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 8099, 46455, 54414, 53763, 67076, 67102, 44181, 67084FL, or 67084alt polypeptide.

In another aspect, the invention pertains to a combination of two or more of the assays described herein. For example, a modulating agent can be identified using a cell-based or a cell free assay, and the ability of the agent to modulate the activity of a 8099, 46455, 54414, 53763, 67076, 67102, 44181, 67084FL, or 67084alt polypeptide can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., a 8099, 46455, 54414, 53763, 67076, 67102, 44181, 67084FL, or 67084alt modulating agent, an antisense 8099, 46455, 54414, 53763, 67076, 67102, 44181, 67084FL, or 67084alt nucleic acid molecule, a 8099, 46455, 54414, 53763, 67076, 67102, 44181, 67084FL, or 67084alt-specific antibody, or a 8099, 46455, 54414, 53763, 67076, 67102, 44181, 67084FL, or 67084alt-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the 8099, 46455, 54414, 53763, 67076, 67102, 44181, 67084FL, or 67084alt nucleotide sequences, described herein, can be used to map the location of the 8099, 46455, 54414, 53763, 67076, 67102, 44181, 67084FL, or 67084alt genes on a chromosome. The mapping of the 8099, 46455, 54414, 53763, 67076, 67102, 44181, 67084FL, or 67084alt sequences to chromosomes is an important first step in correlating these sequences with genes associated with disease.

Briefly, 8099, 46455, 54414, 53763, 67076, 67102, 44181, 67084FL, or 67084alt genes can be mapped to chromosomes by preparing PCR primers (preferably 15-25 bp in length) from the 8099, 46455, 54414, 53763, 67076, 67102, 44181, 67084FL, or 67084alt nucleotide sequences. Computer analysis of the 8099, 46455, 54414, 53763, 67076, 67102, 44181, 67084FL, or 67084alt sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the 8099, 46455, 54414, 53763, 67076, 67102, 44181, 67084FL, or 67084alt sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the 8099, 46455, 54414, 53763, 67076, 67102, 44181, 67084FL, or 67084alt nucleotide sequences to design oligonucleotide primers, sublocalization can be achieved with panels of fragments from specific chromosomes. Other mapping strategies which can similarly be used to map a 8099, 46455, 54414, 53763, 67076, 67102, 44181, 67084FL, or 67084alt sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 8099, 46455, 54414, 53763, 67076, 67102, 44181, 67084FL, or 67084alt gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The 8099, 46455, 54414, 53763, 67076, 67102, 44181, 67084FL, or 67084alt sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the 8099, 46455, 54414, 53763, 67076, 67102, 44181, 67084FL, or 67084alt nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The 8099, 46455, 54414, 53763, 67076, 67102, 44181, 67084FL, or 67084alt nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:46, 49, 52, 55, 58, 61, 64, 67, or 70 can comfortably provide positive individual identification with a panel of perhaps 10 to 1,000 primers which each yield a noncoding amplified sequence of 100 bases. If predicted coding sequences, such as those in SEQ ID NO:48, 51, 54, 57, 60, 63, 66, 69, or 72 are used, a more appropriate number of primers for positive individual identification would be 500-2,000.

If a panel of reagents from 8099, 46455, 54414, 53763, 67076, 67102, 44181, 67084FL, or 67084alt nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of 8099, 46455, 54414, 53763, 67076, 67102, 44181, 67084FL, or 67084alt Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:46, 49, 52, 55, 58, 61, 64, 67, or 70 are particularly appropriate for this use as greater numbers of polymorphisms occur in the noncoding regions, making it easier to differentiate individuals using this technique. Examples of polynucleotide reagents include the 8099, 46455, 54414, 53763, 67076, 67102, 44181, 67084FL, or 67084alt nucleotide sequences or portions thereof, e.g., fragments derived from the noncoding regions of SEQ ID NO:46, 49, 52, 55, 58, 61, 64, 67, or 70, having a length of at least 20 bases, preferably at least 30 bases.

The 8099, 46455, 54414, 53763, 67076, 67102, 44181, 67084FL, or 67084alt nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such 8099, 46455, 54414, 53763, 67076, 67102, 44181, 67084FL, or 67084alt probes can be used to identify tissue by species and/or by organ type.

In a similar fashion, these reagents, e.g., 8099, 46455, 54414, 53763, 67076, 67102, 44181, 67084FL, or 67084alt primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 8099, 46455, 54414, 53763, 67076, 67102, 44181, 67084FL, or 67084alt polypeptide and/or nucleic acid expression as well as 8099, 46455, 54414, 53763, 67076, 67102, 44181, 67084FL, or 67084alt activity, in the context of a biological sample (e.g., blood, serum, cells, tissue) to thereby determine whether an individual is afflicted with a disease or disorder, or is at risk of developing a disorder, associated with aberrant or unwanted 8099, 46455, 54414, 53763, 67076, 67102, 44181, 67084FL, or 67084alt expression or activity. The invention also provides for prognostic (or predictive) assays for determining whether an individual is at risk of developing a disorder associated with 8099, 46455, 54414, 53763, 67076, 67102, 44181, 67084FL, or 67084alt polypeptide, nucleic acid expression or activity. For example, mutations in a 8099, 46455, 54414, 53763, 67076, 67102, 44181, 67084FL, or 67084alt gene can be assayed in a biological sample. Such assays can be used for prognostic or predictive purpose to thereby prophylactically treat an individual prior to the onset of a disorder characterized by or associated with 8099, 46455, 54414, 53763, 67076, 67102, 44181, 67084FL, or 67084alt polypeptide, nucleic acid expression or activity.

Another aspect of the invention pertains to monitoring the influence of agents (e.g., drugs, compounds) on the expression or activity of 8099, 46455, 54414, 53763, 67076, 67102, 44181, 67084FL, or 67084alt in clinical trials.

These and other agents are described in further detail in the following sections.

1. Diagnostic Assays

An exemplary method for detecting the presence or absence of 8099, 46455, 54414, 53763, 67076, 67102, 44181, 67084FL, or 67084alt polypeptide or nucleic acid in a biological sample involves obtaining a biological sample from a test subject and contacting the biological sample with a compound or an agent capable of detecting 8099, 46455, 54414, 53763, 67076, 67102, 44181, 67084FL, or 67084alt polypeptide or nucleic acid (e.g., mRNA, or genomic DNA) that encodes 8099, 46455, 54414, 53763, 67076, 67102, 44181, 67084FL, or 67084alt polypeptide such that the presence of 8099, 46455, 54414, 53763, 67076, 67102, 44181, 67084FL, or 67084alt polypeptide or nucleic acid is detected in the biological sample. In another aspect, the present invention provides a method for detecting the presence of 8099, 46455, 54414, 53763, 67076, 67102, 44181, 67084FL, or 67084alt activity in a biological sample by contacting the biological sample with an agent capable of detecting an indicator of 8099, 46455, 54414, 53763, 67076, 67102, 44181, 67084FL, or 67084alt activity such that the presence of 8099, 46455, 54414, 53763, 67076, 67102, 44181, 67084FL, or 67084alt activity is detected in the biological sample. A preferred agent for detecting 8099, 46455, 54414, 53763, 67076, 67102, 44181, 67084FL, or 67084alt mRNA or genomic DNA is a labeled nucleic acid probe capable of hybridizing to 8099, 46455, 54414, 53763, 67076, 67102, 44181, 67084FL, or 67084alt mRNA or genomic DNA. The nucleic acid probe can be, for example, the 8099, 46455, 54414, 53763, 67076, 67102, 44181, 67084FL, or 67084alt nucleic acid set forth in SEQ ID NO:46, 48, 49, 51, 52, 54, 55, 57, 58, 60, 61, 63, 64, 66, 67, 69, 70, or 72, or the DNA insert of the plasmid deposited with ATCC as Accession Number ______, ______, ______, ______, or ______, or a portion thereof, such as an oligonucleotide of at least 15, 30, 50, 100, 250 or 500 nucleotides in length and sufficient to specifically hybridize under stringent conditions to 8099, 46455, 54414, 53763, 67076, 67102, 44181, 67084FL, or 67084alt mRNA or genomic DNA. Other suitable probes for use in the diagnostic assays of the invention are described herein.

A preferred agent for detecting 8099, 46455, 54414, 53763, 67076, 67102, 44181, 67084FL, or 67084alt polypeptide is an antibody capable of binding to 8099, 46455, 54414, 53763, 67076, 67102, 44181, 67084FL, or 67084alt polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect 8099, 46455, 54414, 53763, 67076, 67102, 44181, 67084FL, or 67084alt mRNA, polypeptide, or genomic DNA in a biological sample in vitro as well as in vivo. For example, in vitro techniques for detection of 8099, 46455, 54414, 53763, 67076, 67102, 44181, 67084FL, or 67084alt mRNA include Northern hybridizations and in situ hybridizations. In vitro techniques for detection of 8099, 46455, 54414, 53763, 67076, 67102, 44181, 67084FL, or 67084alt polypeptide include enzyme linked immunosorbent assays (ELISAs), Western blots, immunoprecipitations and immunofluorescence. In vitro techniques for detection of 8099, 46455, 54414, 53763, 67076, 67102, 44181, 67084FL, or 67084alt genomic DNA include Southern hybridizations. Furthermore, in vivo techniques for detection of PLTR polypeptide include introducing into a subject a labeled 8099, 46455, 54414, 53763, 67076, 67102, 44181, 67084FL, or 67084alt antibody. For example, the antibody can be labeled with a radioactive marker whose presence and location in a subject can be detected by standard imaging techniques.

The present invention also provides diagnostic assays for identifying the presence or absence of a genetic alteration characterized by at least one of (i) aberrant modification or mutation of a gene encoding a 8099, 46455, 54414, 53763, 67076, 67102, 44181, 67084FL, or 67084alt polypeptide; (ii) aberrant expression of a gene encoding a 8099, 46455, 54414, 53763, 67076, 67102, 44181, 67084FL, or 67084alt polypeptide; (iii) mis-regulation of the gene; and (iii) aberrant post-translational modification of a 8099, 46455, 54414, 53763, 67076, 67102, 44181, 67084FL, or 67084alt polypeptide, wherein a wild-type form of the gene encodes a polypeptide with a 8099, 46455, 54414, 53763, 67076, 67102, 44181, 67084FL, or 67084alt activity. “Misexpression or aberrant expression”, as used herein, refers to a non-wild type pattern of gene expression, at the RNA or protein level. It includes, but is not limited to, expression at non-wild type levels (e.g., over or under expression); a pattern of expression that differs from wild type in terms of the time or stage at which the gene is expressed (e.g., increased or decreased expression (as compared with wild type) at a predetermined developmental period or stage); a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene (e.g., a pattern of increased or decreased expression (as compared with wild type) in the presence of an increase or decrease in the strength of the stimulus).

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting 8099, 46455, 54414, 53763, 67076, 67102, 44181, 67084FL, or 67084alt polypeptide, mRNA, or genomic DNA, such that the presence of 8099, 46455, 54414, 53763, 67076, 67102, 44181, 67084FL, or 67084alt polypeptide, mRNA or genomic DNA is detected in the biological sample, and comparing the presence of 8099, 46455, 54414, 53763, 67076, 67102, 44181, 67084FL, or 67084alt polypeptide, mRNA or genomic DNA in the control sample with the presence of 8099, 46455, 54414, 53763, 67076, 67102, 44181, 67084FL, or 67084alt polypeptide, mRNA or genomic DNA in the test sample.

The invention also encompasses kits for detecting the presence of 8099, 46455, 54414, 53763, 67076, 67102, 44181, 67084FL, or 67084alt in a biological sample. For example, the kit can comprise a labeled compound or agent capable of detecting 8099, 46455, 54414, 53763, 67076, 67102, 44181, 67084FL, or 67084alt polypeptide or mRNA in a biological sample; means for determining the amount of 8099, 46455, 54414, 53763, 67076, 67102, 44181, 67084FL, or 67084alt in the sample; and means for comparing the amount of 8099, 46455, 54414, 53763, 67076, 67102, 44181, 67084FL, or 67084alt in the sample with a standard. The compound or agent can be packaged in a suitable container. The kit can further comprise instructions for using the kit to detect 8099, 46455, 54414, 53763, 67076, 67102, 44181, 67084FL, or 67084alt polypeptide or nucleic acid.

2. Prognostic Assays

The diagnostic methods described herein can furthermore be utilized to identify subjects having or at risk of developing a disease or disorder associated with aberrant or unwanted 8099, 46455, 54414, 53763, 67076, 67102, 44181, 67084FL, or 67084alt expression or activity. As used herein, the term “aberrant” includes a 8099, 46455, 54414, 53763, 67076, 67102, 44181, 67084FL, or 67084alt expression or activity which deviates from the wild type 8099, 46455, 54414, 53763, 67076, 67102, 44181, 67084FL, or 67084alt expression or activity. Aberrant expression or activity includes increased or decreased expression or activity, as well as expression or activity which does not follow the wild type developmental pattern of expression or the subcellular pattern of expression. For example, aberrant 8099, 46455, 54414, 53763, 67076, 67102, 44181, 67084FL, or 67084alt expression or activity is intended to include the cases in which a mutation in the 8099, 46455, 54414, 53763, 67076, 67102, 44181, 67084FL, or 67084alt gene causes the 8099, 46455, 54414, 53763, 67076, 67102, 44181, 67084FL, or 67084alt gene to be under-expressed or over-expressed and situations in which such mutations result in a non-functional 8099, 46455, 54414, 53763, 67076, 67102, 44181, 67084FL, or 67084alt polypeptide or a polypeptide which does not function in a wild-type fashion, e.g., a protein which does not interact with or transport a 8099, 46455, 54414, 53763, 67076, 67102, 44181, 67084FL, or 67084alt substrate, or one which interacts with or transports a non-8099, 46455, 54414, 53763, 67076, 67102, 44181, 67084FL, or 67084alt substrate. As used herein, the term “unwanted” includes an unwanted phenomenon involved in a biological response such as deregulated cellular proliferation. For example, the term unwanted includes a 8099, 46455, 54414, 53763, 67076, 67102, 44181, 67084FL, or 67084alt expression or activity which is undesirable in a subject.

The assays described herein, such as the preceding diagnostic assays or the following assays, can be utilized to identify a subject having or at risk of developing a disorder associated with a misregulation in 8099, 46455, 54414, 53763, 67076, 67102, 44181, 67084FL, or 67084alt polypeptide activity or nucleic acid expression, such as a as a cell growth, proliferation and/or differentiation disorder. Alternatively, the prognostic assays can be utilized to identify a subject having or at risk for developing a disorder associated with a misregulation in 8099, 46455, 54414, 53763, 67076, 67102, 44181, 67084FL, or 67084alt polypeptide activity or nucleic acid expression, such as a cell growth, proliferation and/or differentiation disorder, a sugar transporter associated disorder, or a potassium channel associated disorder, as described herein. Thus, the present invention provides a method for identifying a disease or disorder associated with aberrant or unwanted 8099, 46455, 54414, 53763, 67076, 67102, 44181, 67084FL, or 67084alt expression or activity in which a test sample is obtained from a subject and 8099, 46455, 54414, 53763, 67076, 67102, 44181, 67084FL, or 67084alt polypeptide or nucleic acid (e.g., mRNA or genomic DNA) is detected, wherein the presence of 8099, 46455, 54414, 53763, 67076, 67102, 44181, 67084FL, or 67084alt polypeptide or nucleic acid is diagnostic for a subject having or at risk of developing a disease or disorder associated with aberrant or unwanted 8099, 46455, 54414, 53763, 67076, 67102, 44181, 67084FL, or 67084alt expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 8099, 46455, 54414, 53763, 67076, 67102, 44181, 67084FL, or 67084alt expression or activity. For example, such methods can be used to determine whether a subject can be effectively treated with an agent for a sugar transporter-associated disorder, a potassium channel associated disorder, or phospholipid transporter-associated disorder. Thus, the present invention provides methods for determining whether a subject can be effectively treated with an agent for a disorder associated with aberrant or unwanted 8099, 46455, 54414, 53763, 67076, 67102, 44181, 67084FL, or 67084alt expression or activity in which a test sample is obtained and 8099, 46455, 54414, 53763, 67076, 67102, 44181, 67084FL, or 67084alt polypeptide or nucleic acid expression or activity is detected (e.g., wherein the abundance of 8099, 46455, 54414, 53763, 67076, 67102, 44181, 67084FL, or 67084alt polypeptide or nucleic acid expression or activity is diagnostic for a subject that can be administered the agent to treat a disorder associated with aberrant or unwanted 8099, 46455, 54414, 53763, 67076, 67102, 44181, 67084FL, or 67084alt expression or activity).

The methods of the invention can also be used to detect genetic alterations in a 8099, 46455, 54414, 53763, 67076, 67102, 44181, 67084FL, or 67084alt gene, thereby determining if a subject with the altered gene is at risk for a disorder characterized by misregulation in 8099, 46455, 54414, 53763, 67076, 67102, 44181, 67084FL, or 67084alt polypeptide activity or nucleic acid expression, such as a cell growth, proliferation and/or differentiation disorder. In preferred embodiments, the methods include detecting, in a sample of cells from the subject, the presence or absence of a genetic alteration characterized by at least one of an alteration affecting the integrity of a gene encoding a 8099, 46455, 54414, 53763, 67076, 67102, 44181, 67084FL, or 67084alt-polypeptide, or the mis-expression of the 8099, 46455, 54414, 53763, 67076, 67102, 44181, 67084FL, or 67084alt gene. For example, such genetic alterations can be detected by ascertaining the existence of at least one of 1) a deletion of one or more nucleotides from a 8099, 46455, 54414, 53763, 67076, 67102, 44181, 67084FL, or 67084alt gene; 2) an addition of one or more nucleotides to a 8099, 46455, 54414, 53763, 67076, 67102, 44181, 67084FL, or 67084alt gene; 3) a substitution of one or more nucleotides of a 8099, 46455, 54414, 53763, 67076, 67102, 44181, 67084FL, or 67084alt gene, 4) a chromosomal rearrangement of a 8099, 46455, 54414, 53763, 67076, 67102, 44181, 67084FL, or 67084alt gene; 5) an alteration in the level of a messenger RNA transcript of a 8099, 46455, 54414, 53763, 67076, 67102, 44181, 67084FL, or 67084alt gene, 6) aberrant modification of a 8099, 46455, 54414, 53763, 67076, 67102, 44181, 67084FL, or 67084alt gene, such as of the methylation pattern of the genomic DNA, 7) the presence of a non-wild type splicing pattern of a messenger RNA transcript of a 8099, 46455, 54414, 53763, 67076, 67102, 44181, 67084FL, or 67084alt gene, 8) a non-wild type level of a 8099, 46455, 54414, 53763, 67076, 67102, 44181, 67084FL, or 67084alt-polypeptide, 9) allelic loss of a 8099, 46455, 54414, 53763, 67076, 67102, 44181, 67084FL, or 67084alt gene, and 10) inappropriate post-translational modification of a 8099, 46455, 54414, 53763, 67076, 67102, 44181, 67084FL, or 67084alt-polypeptide. As described herein, there are a large number of assays known in the art which can be used for detecting alterations in a 8099, 46455, 54414, 53763, 67076, 67102, 44181, 67084FL, or 67084alt gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the 8099, 46455, 54414, 53763, 67076, 67102, 44181, 67084FL, or 67084alt gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a 8099, 46455, 54414, 53763, 67076, 67102, 44181, 67084FL, or 67084alt gene under conditions such that hybridization and amplification of the 8099, 46455, 54414, 53763, 67076, 67102, 44181, 67084FL, or 67084alt gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 8099, 46455, 54414, 53763, 67076, 67102, 44181, 67084FL, or 67084alt gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 8099, 46455, 54414, 53763, 67076, 67102, 44181, 67084FL, or 67084alt can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 8099, 46455, 54414, 53763, 67076, 67102, 44181, 67084FL, or 67084alt can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 8099, 46455, 54414, 53763, 67076, 67102, 44181, 67084FL, or 67084alt gene and detect mutations by comparing the sequence of the sample 8099, 46455, 54414, 53763, 67076, 67102, 44181, 67084FL, or 67084alt with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g, PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 8099, 46455, 54414, 53763, 67076, 67102, 44181, 67084FL, or 67084alt gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type 8099, 46455, 54414, 53763, 67076, 67102, 44181, 67084FL, or 67084alt sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 8099, 46455, 54414, 53763, 67076, 67102, 44181, 67084FL, or 67084alt cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 8099, 46455, 54414, 53763, 67076, 67102, 44181, 67084FL, or 67084alt sequence, e.g, a wild-type 8099, 46455, 54414, 53763, 67076, 67102, 44181, 67084FL, or 67084alt sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 8099, 46455, 54414, 53763, 67076, 67102, 44181, 67084FL, or 67084alt genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 8099, 46455, 54414, 53763, 67076, 67102, 44181, 67084FL, or 67084alt nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 8099, 46455, 54414, 53763, 67076, 67102, 44181, 67084FL, or 67084alt gene.

Furthermore, any cell type or tissue in which 8099, 46455, 54414, 53763, 67076, 67102, 44181, 67084FL, or 67084alt is expressed may be utilized in the prognostic assays described herein.

3. Monitoring of Effects During Clinical Trials

Monitoring the influence of agents (e.g., drugs) on the expression or activity of a 8099, 46455, 54414, 53763, 67076, 67102, 44181, 67084FL, or 67084alt polypeptide (e.g., the modulation of gene expression, cellular signaling, 8099, 46455, 54414, 53763, 67076, 67102, 44181, 67084FL, or 67084alt activity, phospholipid transporter activity, and/or cell growth, proliferation, differentiation, absorption, and/or secretion mechanisms) can be applied not only in basic drug screening, but also in clinical trials. For example, the effectiveness of an agent determined by a screening assay as described herein to increase 8099, 46455, 54414, 53763, 67076, 67102, 44181, 67084FL, or 67084alt gene expression, polypeptide levels, or upregulate 8099, 46455, 54414, 53763, 67076, 67102, 44181, 67084FL, or 67084alt activity, can be monitored in clinical trials of subjects exhibiting decreased 8099, 46455, 54414, 53763, 67076, 67102, 44181, 67084FL, or 67084alt gene expression, polypeptide levels, or downregulated 8099, 46455, 54414, 53763, 67076, 67102, 44181, 67084FL, or 67084alt activity. Alternatively, the effectiveness of an agent determined by a screening assay to decrease 8099, 46455, 54414, 53763, 67076, 67102, 44181, 67084FL, or 67084alt gene expression, polypeptide levels, or downregulate 8099, 46455, 54414, 53763, 67076, 67102, 44181, 67084FL, or 67084alt activity, can be monitored in clinical trials of subjects exhibiting increased 8099, 46455, 54414, 53763, 67076, 67102, 44181, 67084FL, or 67084alt gene expression, polypeptide levels, or upregulated 8099, 46455, 54414, 53763, 67076, 67102, 44181, 67084FL, or 67084alt activity. In such clinical trials, the expression or activity of a 8099, 46455, 54414, 53763, 67076, 67102, 44181, 67084FL, or 67084alt gene, and preferably, other genes that have been implicated in, for example, a 8099, 46455, 54414, 53763, 67076, 67102, 44181, 67084FL, or 67084alt-associated disorder can be used as a “read out” or markers of the phenotype of a particular cell.

For example, and not by way of limitation, genes, including 8099, 46455, 54414, 53763, 67076, 67102, 44181, 67084FL, or 67084alt, that are modulated in cells by treatment with an agent (e.g., compound, drug or small molecule) which modulates 67076, 67102, 44181, 67084FL, or 67084alt activity (e.g., identified in a screening assay as described herein) can be identified.

Thus, to study the effect of agents on phospholipid transporter-associated disorders (e.g., disorders characterized by deregulated gene expression, cellular signaling, 67076, 67102, 44181, 67084FL, or 67084alt activity, phospholipid transporter activity, and/or cell growth, proliferation, differentiation, absorption, and/or secretion mechanisms), for example, in a clinical trial, cells can be isolated and RNA prepared and analyzed for the levels of expression of 67076, 67102, 44181, 67084FL, or 67084alt and other genes implicated in the transporter-associated disorder, respectively. The levels of gene expression (e.g., a gene expression pattern) can be quantified by northern blot analysis or RT-PCR, as described herein, or alternatively by measuring the amount of polypeptide produced, by one of the methods as described herein, or by measuring the levels of activity of 67076, 67102, 44181, 67084FL, or 67084alt or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a 8099, 46455, 54414, 53763, 67076, 67102, 44181, 67084FL, or 67084alt polypeptide, mRNA, or genomic DNA in the preadministration sample; (iii) obtaining one or more post-administration samples from the subject; (iv) detecting the level of expression or activity of the 8099, 46455, 54414, 53763, 67076, 67102, 44181, 67084FL, or 67084alt polypeptide, mRNA, or genomic DNA in the post-administration samples; (v) comparing the level of expression or activity of the 8099, 46455, 54414, 53763, 67076, 67102, 44181, 67084FL, or 67084alt polypeptide, mRNA, or genomic DNA in the pre-administration sample with the 8099, 46455, 54414, 53763, 67076, 67102, 44181, 67084FL, or 67084alt polypeptide, mRNA, or genomic DNA in the post administration sample or samples; and (vi) altering the administration of the agent to the subject accordingly. For example, increased administration of the agent may be desirable to increase the expression or activity of 8099, 46455, 54414, 53763, 67076, 67102, 44181, 67084FL, or 67084alt to higher levels than detected, i.e., to increase the effectiveness of the agent. Alternatively, decreased administration of the agent may be desirable to decrease expression or activity of 8099, 46455, 54414, 53763, 67076, 67102, 44181, 67084FL, or 67084alt to lower levels than detected, i.e. to decrease the effectiveness of the agent. According to such an embodiment, 8099, 46455, 54414, 53763, 67076, 67102, 44181, 67084FL, or 67084alt expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted 8099, 46455, 54414, 53763, 67076, 67102, 44181, 67084FL, or 67084alt expression or activity, e.g. a phospholipid transporter-associated disorder. “Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of disease or disorder or the predisposition toward a disease or disorder. A therapeutic agent includes, but is not limited to, small molecules, peptides, antibodies, ribozymes and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the 8099, 46455, 54414, 53763, 67076, 67102, 44181, 67084FL, or 67084alt molecules of the present invention or 8099, 46455, 54414, 53763, 67076, 67102, 44181, 67084FL, or 67084alt modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted 8099, 46455, 54414, 53763, 67076, 67102, 44181, 67084FL, or 67084alt expression or activity, by administering to the subject a 8099, 46455, 54414, 53763, 67076, 67102, 44181, 67084FL, or 67084alt or an agent which modulates 8099, 46455, 54414, 53763, 67076, 67102, 44181, 67084FL, or 67084alt expression or at least one 8099, 46455, 54414, 53763, 67076, 67102, 44181, 67084FL, or 67084alt activity. Subjects at risk for a disease which is caused or contributed to by aberrant or unwanted 8099, 46455, 54414, 53763, 67076, 67102, 44181, 67084FL, or 67084alt expression or activity can be identified by, for example, any or a combination of diagnostic or prognostic assays as described herein. Administration of a prophylactic agent can occur prior to the manifestation of symptoms characteristic of the 8099, 46455, 54414, 53763, 67076, 67102, 44181, 67084FL, or 67084alt aberrancy, such that a disease or disorder is prevented or, alternatively, delayed in its progression. Depending on the type of 8099, 46455, 54414, 53763, 67076, 67102, 44181, 67084FL, or 67084alt aberrancy, for example, a 8099, 46455, 54414, 53763, 67076, 67102, 44181, 67084FL, or 67084alt, 8099, 46455, 54414, 53763, 67076, 67102, 44181, 67084FL, or 67084alt agonist or 8099, 46455, 54414, 53763, 67076, 67102, 44181, 67084FL, or 67084alt antagonist agent can be used for treating the subject. The appropriate agent can be determined based on screening assays described herein.

2. Therapeutic Methods

Another aspect of the invention pertains to methods of modulating 8099, 46455, 54414, 53763, 67076, 67102, 44181, 67084FL, or 67084alt expression or activity for therapeutic purposes. Accordingly, in an exemplary embodiment, the modulatory method of the invention involves contacting a cell capable of expressing 8099, 46455, 54414, 53763, 67076, 67102, 44181, 67084FL, or 67084alt with an agent that modulates one or more of the activities of 8099, 46455, 54414, 53763, 67076, 67102, 44181, 67084FL, or 67084alt polypeptide activity associated with the cell, such that 8099, 46455, 54414, 53763, 67076, 67102, 44181, 67084FL, or 67084alt activity in the cell is modulated. An agent that modulates 8099, 46455, 54414, 53763, 67076, 67102, 44181, 67084FL, or 67084alt polypeptide activity can be an agent as described herein, such as a nucleic acid or a polypeptide, a naturally-occurring target molecule of a 8099, 46455, 54414, 53763, 67076, 67102, 44181, 67084FL, or 67084alt polypeptide (e.g., a 8099, 46455, 54414, 53763, 67076, 67102, 44181, 67084FL, or 67084alt substrate), a 8099, 46455, 54414, 53763, 67076, 67102, 44181, 67084FL, or 67084alt antibody, a 8099, 46455, 54414, 53763, 67076, 67102, 44181, 67084FL, or 67084alt agonist or antagonist, a peptidomimetic of a 8099, 46455, 54414, 53763, 67076, 67102, 44181, 67084FL, or 67084alt agonist or antagonist, or other small molecule. In one embodiment, the agent stimulates one or more 8099, 46455, 54414, 53763, 67076, 67102, 44181, 67084FL, or 67084alt activities. Examples of such stimulatory agents include active 8099, 46455, 54414, 53763, 67076, 67102, 44181, 67084FL, or 67084alt polypeptide and a nucleic acid molecule encoding 8099, 46455, 54414, 53763, 67076, 67102, 44181, 67084FL, or 67084alt that has been introduced into the cell. In another embodiment, the agent inhibits one or more 8099, 46455, 54414, 53763, 67076, 67102, 44181, 67084FL, or 67084alt activities. Examples of such inhibitory agents include antisense 8099, 46455, 54414, 53763, 67076, 67102, 44181, 67084FL, or 67084alt nucleic acid molecules, anti-8099, 46455, 54414, 53763, 67076, 67102, 44181, 67084FL, or 67084alt 3 antibodies, and 8099, 46455, 54414, 53763, 67076, 67102, 44181, 67084FL, or 67084alt inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 8099, 46455, 54414, 53763, 67076, 67102, 44181, 67084FL, or 67084alt polypeptide or nucleic acid molecule. In one embodiment, the method involves administering an agent (e.g., an agent identified by a screening assay described herein), or combination of agents that modulates (e.g., upregulates or downregulates) 8099, 46455, 54414, 53763, 67076, 67102, 44181, 67084FL, or 67084alt expression or activity. In another embodiment, the method involves administering a 8099, 46455, 54414, 53763, 67076, 67102, 44181, 67084FL, or 67084alt polypeptide or nucleic acid molecule as therapy to compensate for reduced, aberrant, or unwanted 8099, 46455, 54414, 53763, 67076, 67102, 44181, 67084FL, or 67084alt expression or activity.

Stimulation of 8099, 46455, 54414, 53763, 67076, 67102, 44181, 67084FL, or 67084alt activity is desirable in situations in which 8099, 46455, 54414, 53763, 67076, 67102, 44181, 67084FL, or 67084alt is abnormally downregulated and/or in which increased 8099, 46455, 54414, 53763, 67076, 67102, 44181, 67084FL, or 67084alt activity is likely to have a beneficial effect. Likewise, inhibition of 8099, 46455, 54414, 53763, 67076, 67102, 44181, 67084FL, or 67084alt activity is desirable in situations in which 8099, 46455, 54414, 53763, 67076, 67102, 44181, 67084FL, or 67084alt is abnormally upregulated and/or in which decreased 8099, 46455, 54414, 53763, 67076, 67102, 44181, 67084FL, or 67084alt activity is likely to have a beneficial effect.

3. Pharmacogenomics

The 8099, 46455, 54414, 53763, 67076, 67102, 44181, 67084FL, or 67084alt molecules of the present invention, as well as agents, or modulators which have a stimulatory or inhibitory effect on 8099, 46455, 54414, 53763, 67076, 67102, 44181, 67084FL, or 67084alt activity (e.g., 8099, 46455, 54414, 53763, 67076, 67102, 44181, 67084FL, or 67084alt gene expression) as identified by a screening assay described herein can be administered to individuals to treat (prophylactically or therapeutically), for example, disorders characterized by aberrant 8099, 46455, 54414, 53763, 67076, 67102, 44181, 67084FL, or 67084alt gene expression, 8099, 46455, 54414, 53763, 67076, 67102, 44181, 67084FL, or 67084alt activity, membrane excitability or conductance, gene transcription, phospholipid transporter activity, cellular signaling, and/or cell growth, proliferation, differentiation, absorption, and/or secretion disorders associated with aberrant or unwanted 8099, 46455, 54414, 53763, 67076, 67102, 44181, 67084FL, or 67084alt activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 8099, 46455, 54414, 53763, 67076, 67102, 44181, 67084FL, or 67084alt molecule or 8099, 46455, 54414, 53763, 67076, 67102, 44181, 67084FL, or 67084alt modulator as well as tailoring the dosage and/or therapeutic regimen of treatment with a 8099, 46455, 54414, 53763, 67076, 67102, 44181, 67084FL, or 67084alt molecule or 8099, 46455, 54414, 53763, 67076, 67102, 44181, 67084FL, or 67084alt modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a 8099,46455, 54414, 53763, 67076, 67102, 44181, 67084FL, or 67084alt polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a 8099, 46455, 54414, 53763, 67076, 67102, 44181, 67084FL, or 67084alt molecule or 8099, 46455, 54414, 53763, 67076, 67102, 44181, 67084FL, or 67084alt modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a 8099, 46455, 54414, 53763, 67076, 67102, 44181, 67084FL, or 67084alt molecule or 8099, 46455, 54414, 53763, 67076, 67102, 44181, 67084FL, or 67084alt modulator, such as a modulator identified by one of the exemplary screening assays described herein.

4. Use of 8099, 46455, 54414, 53763, 67076, 67102, 44181, 67084FL, or 67084alt Molecules as Surrogate Markers

The 8099, 46455, 54414, 53763, 67076, 67102, 44181, 67084FL, or 67084alt molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 8099, 46455, 54414, 53763, 67076, 67102, 44181, 67084FL, or 67084alt molecules of the invention may be detected, and may be correlated with one or more biological states in vivo. For example, the 8099, 46455, 54414, 53763, 67076, 67102, 44181, 67084FL, or 67084alt molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 8099, 46455, 54414, 53763, 67076, 67102, 44181, 67084FL, or 67084alt molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 8099, 46455, 54414, 53763, 67076, 67102, 44181, 67084FL, or 67084alt marker) transcription or expression, the amplified marker may be in a quantity which is more readily detectable than the drug itself. Also, the marker may be more easily detected due to the nature of the marker itself; for example, using the methods described herein, anti-8099, 46455, 54414, 53763, 67076, 67102, 44181, 67084FL, or 67084alt antibodies may be employed in an immune-based detection system for a 8099, 46455, 54414, 53763, 67076, 67102, 44181, 67084FL, or 67084alt polypeptide marker, or 8099, 46455, 54414, 53763, 67076, 67102, 44181, 67084FL, or 67084alt-specific radiolabeled probes may be used to detect a 8099, 46455, 54414, 53763, 67076, 67102, 44181, 67084FL, or 67084alt mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The 8099, 46455, 54414, 53763, 67076, 67102, 44181, 67084FL, or 67084alt molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or polypeptide (e.g., 8099, 46455, 54414, 53763, 67076, 67102, 44181, 67084FL, or 67084alt polypeptide or RNA) for specific tumor markers in a subject, a drug or course of treatment may be selected that is optimized for the treatment of the specific tumor likely to be present in the subject. Similarly, the presence or absence of a specific sequence mutation in 8099, 46455, 54414, 53763, 67076, 67102, 44181, 67084FL, or 67084alt DNA may correlate 8099, 46455, 54414, 53763, 67076, 67102, 44181, 67084FL, or 67084alt drug response. The use of pharmacogenomic markers therefore permits the application of the most appropriate treatment for each subject without having to administer the therapy.

VI. Electronic Apparatus Readable Media and Arrays

Electronic apparatus readable media comprising 8099, 46455, 54414, 53763, 67076, 67102, 44181, 67084FL, or 67084alt sequence information is also provided. As used herein, “8099, 46455, 54414, 53763, 67076, 67102, 44181, 67084FL, or 67084alt sequence information” refers to any nucleotide and/or amino acid sequence information particular to the 8099, 46455, 54414, 53763, 67076, 67102, 44181, 67084FL, or 67084alt molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said 8099, 46455, 54414, 53763, 67076, 67102, 44181, 67084FL, or 67084alt sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon 8099, 46455, 54414, 53763, 67076, 67102, 44181, 67084FL, or 67084alt sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the 8099, 46455, 54414, 53763, 67076, 67102, 44181, 67084FL, or 67084alt sequence information.

A variety of software programs and formats can be used to store the sequence information on the electronic apparatus readable medium. For example, the sequence information can be represented in a word processing text file, formatted in commercially-available software such as WordPerfect and Microsoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the 8099, 46455, 54414, 53763, 67076, 67102, 44181, 67084FL, or 67084alt sequence information.

By providing 8099, 46455, 54414, 53763, 67076, 67102, 44181, 67084FL, or 67084alt sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a 8099, 46455, 54414, 53763, 67076, 67102, 44181, 67084FL, or 67084alt-associated disease or disorder or a pre-disposition to a 8099, 46455, 54414, 53763, 67076, 67102, 44181, 67084FL, or 67084alt-associated disease or disorder, wherein the method comprises the steps of determining 8099, 46455, 54414, 53763, 67076, 67102, 44181, 67084FL, or 67084alt sequence information associated with the subject and based on the 8099, 46455, 54414, 53763, 67076, 67102, 44181, 67084FL, or 67084alt sequence information, determining whether the subject has a 8099, 46455, 54414, 53763, 67076, 67102, 44181, 67084FL, or 67084alt-associated disease or disorder or a pre-disposition to a 8099, 46455, 54414, 53763, 67076, 67102, 44181, 67084FL, or 67084alt-associated disease or disorder and/or recommending a particular treatment for the disease, disorder or pre-disease condition.

The present invention further provides in an electronic system and/or in a network, a method for determining whether a subject has a 8099, 46455, 54414, 53763, 67076, 67102, 44181, 67084FL, or 67084alt-associated disease or disorder or a pre-disposition to a disease associated with a 8099, 46455, 54414, 53763, 67076, 67102, 44181, 67084FL, or 67084alt wherein the method comprises the steps of determining 8099, 46455, 54414, 53763, 67076, 67102, 44181, 67084FL, or 67084alt sequence information associated with the subject, and based on the 8099, 46455, 54414, 53763, 67076, 67102, 44181, 67084FL, or 67084alt sequence information, determining whether the subject has a 8099, 46455, 54414, 53763, 67076, 67102, 44181, 67084FL, or 67084alt-associated disease or disorder or a pre-disposition to a 8099, 46455, 54414, 53763, 67076, 67102, 44181, 67084FL, or 67084alt-associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a 8099, 46455, 54414, 53763, 67076, 67102, 44181, 67084FL, or 67084alt-associated disease or disorder or a pre-disposition to a 8099, 46455, 54414, 53763, 67076, 67102, 44181, 67084FL, or 67084alt-associated disease or disorder associated with 8099, 46455, 54414, 53763, 67076, 67102, 44181, 67084FL, or 67084alt, said method comprising the steps of receiving 8099, 46455, 54414, 53763, 67076, 67102, 44181, 67084FL, or 67084alt sequence information from the subject and/or information related thereto, receiving phenotypic information associated with the subject, acquiring information from the network corresponding to 8099, 46455, 54414, 53763, 67076, 67102, 44181, 67084FL, or 67084alt and/or a 8099, 46455, 54414, 53763, 67076, 67102, 44181, 67084FL, or 67084alt-associated disease or disorder, and based on one or more of the phenotypic information, the 8099, 46455, 54414, 53763, 67076, 67102, 44181, 67084FL, or 67084alt information (e.g., sequence information and/or information related thereto), and the acquired information, determining whether the subject has a 8099, 46455, 54414, 53763, 67076, 67102, 44181, 67084FL, or 67084alt-associated disease or disorder or a pre-disposition to a 8099, 46455, 54414, 53763, 67076, 67102, 44181, 67084FL, or 67084alt-associated disease or disorder. The method may further comprise the step of recommending a particular treatment for the disease, disorder or pre-disease condition.

The present invention also provides a business method for determining whether a subject has a 8099, 46455, 54414, 53763, 67076, 67102, 44181, 67084FL, or 67084alt-associated disease or disorder or a pre-disposition to a 8099, 46455, 54414, 53763, 67076, 67102, 44181, 67084FL, or 67084alt-associated disease or disorder, said method comprising the steps of receiving information related to 8099, 46455, 54414, 53763, 67076, 67102, 44181, 67084FL, or 67084alt (e.g., sequence information and/or information related thereto), receiving phenotypic information associated with the subject, acquiring information from the network related to 8099, 46455, 54414, 53763, 67076, 67102, 44181, 67084FL, or 67084alt and/or related to a 8099, 46455, 54414, 53763, 67076, 67102, 44181, 67084FL, or 67084alt-associated disease or disorder, and based on one or more of the phenotypic information, the 8099, 46455, 54414, 53763, 67076, 67102, 44181, 67084FL, or 67084alt information, and the acquired information, determining whether the subject has a 8099, 46455, 54414, 53763, 67076, 67102, 44181, 67084FL, or 67084alt-associated disease or disorder or a pre-disposition to a 8099, 46455, 54414, 53763, 67076, 67102, 44181, 67084FL, or 67084alt-associated disease or disorder. The method may further comprise the step of recommending a particular treatment for the disease, disorder or pre-disease condition.

The invention also includes an array comprising a 8099, 46455, 54414, 53763, 67076, 67102, 44181, 67084FL, or 67084alt sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be 8099, 46455, 54414, 53763, 67076, 67102, 44181, 67084FL, or 67084alt. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a 8099, 46455, 54414, 53763, 67076, 67102, 44181, 67084FL, or 67084alt-associated disease or disorder, progression of 8099, 46455, 54414, 53763, 67076, 67102, 44181, 67084FL, or 67084alt-associated disease or disorder, and processes, such a cellular transformation associated with the 8099, 46455, 54414, 53763, 67076, 67102, 44181, 67084FL, or 67084alt-associated disease or disorder.

The array is also useful for ascertaining the effect of the expression of a gene on the expression of other genes in the same cell or in different cells (e.g., ascertaining the effect of 8099, 46455, 54414, 53763, 67076, 67102, 44181, 67084FL, or 67084alt expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including 8099, 46455, 54414, 53763, 67076, 67102, 44181, 67084FL, or 67084alt ) that could serve as a molecular target for diagnosis or therapeutic intervention.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human 8099, 46455, 54414, 53763, 67076, 67102, 44181, 67084FL, and 67084alt cDNAs

In this example, the identification and characterization of the gene encoding human 8099, 46455, 54414, 53763, 67076, 67102, 44181, full length 67084 (67084FL), and 67084alt is described.

Isolation of the Human 8099 and 46455 cDNAs

The invention is based, at least in part, on the discovery of a human gene encoding a novel polypeptide, referred to herein as human 8099. The entire sequence of the human clone 8099 was determined and found to contain an open reading frame termed human “8099.” The nucleotide sequence of the human 8099 gene is set forth in FIGS. 44A-44D and in the Sequence Listing as SEQ ID NO:46. The amino acid sequence of the human 8099 expression product is set forth in FIGS. 44A-D and in the Sequence Listing as SEQ ID NO:47. The 8099 polypeptide comprises 617 amino acids. The coding region (open reading frame) of SEQ ID NO:46 is set forth as SEQ ID NO:48. Clone 8099, comprising the coding region of human 8099, was deposited with the American Type Culture Collection (ATCC®), 10801 University Boulevard, Manassas, Va. 20110-2209, on ______, and assigned Accession No. ______.

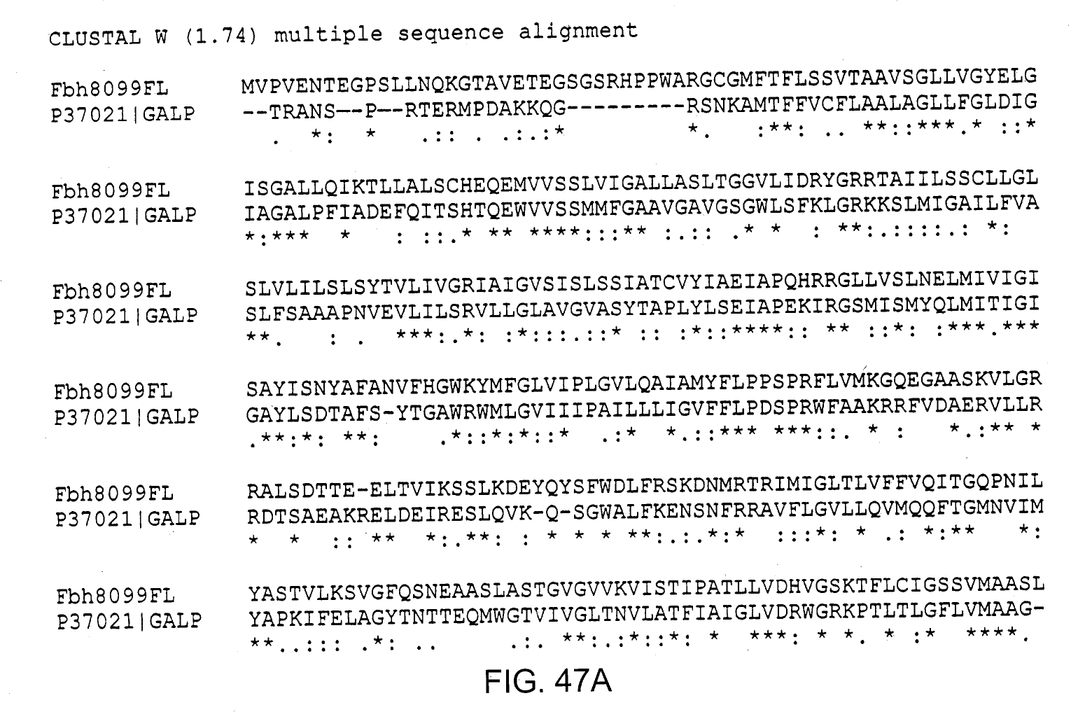

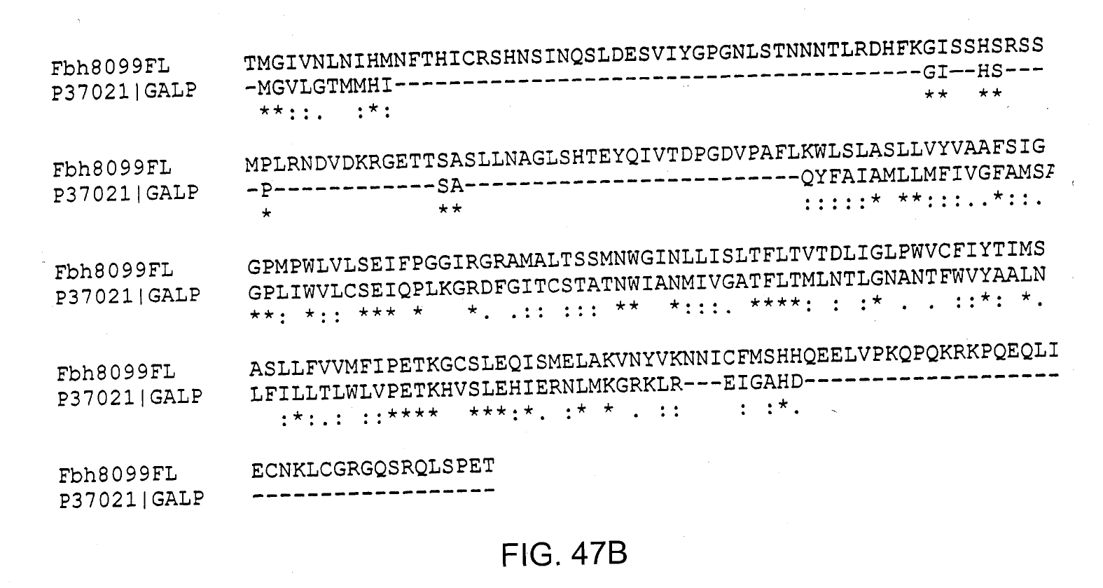

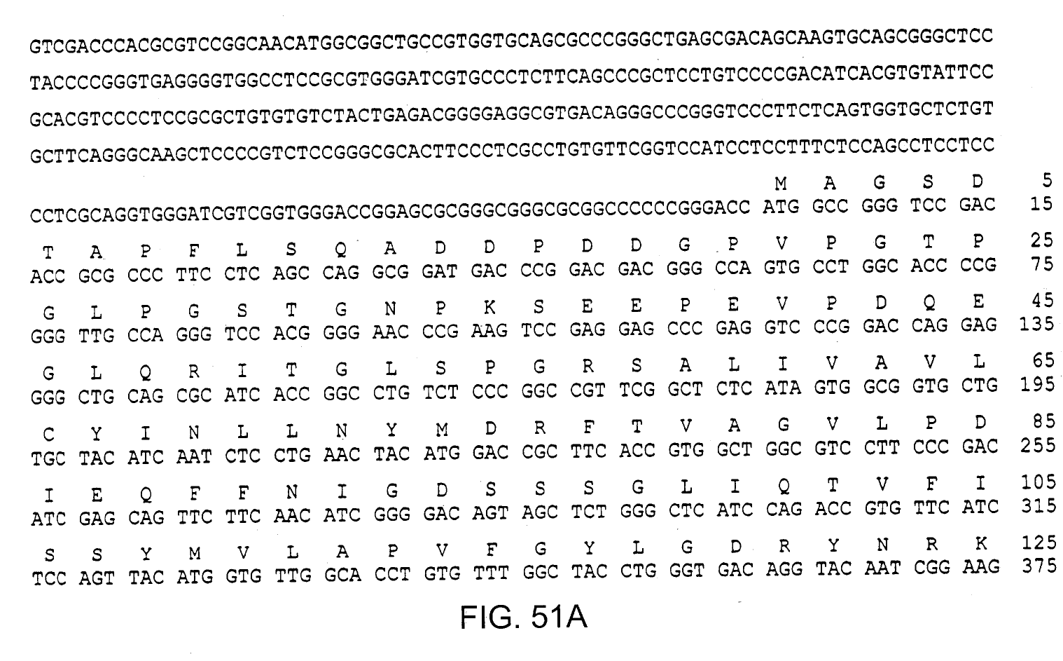

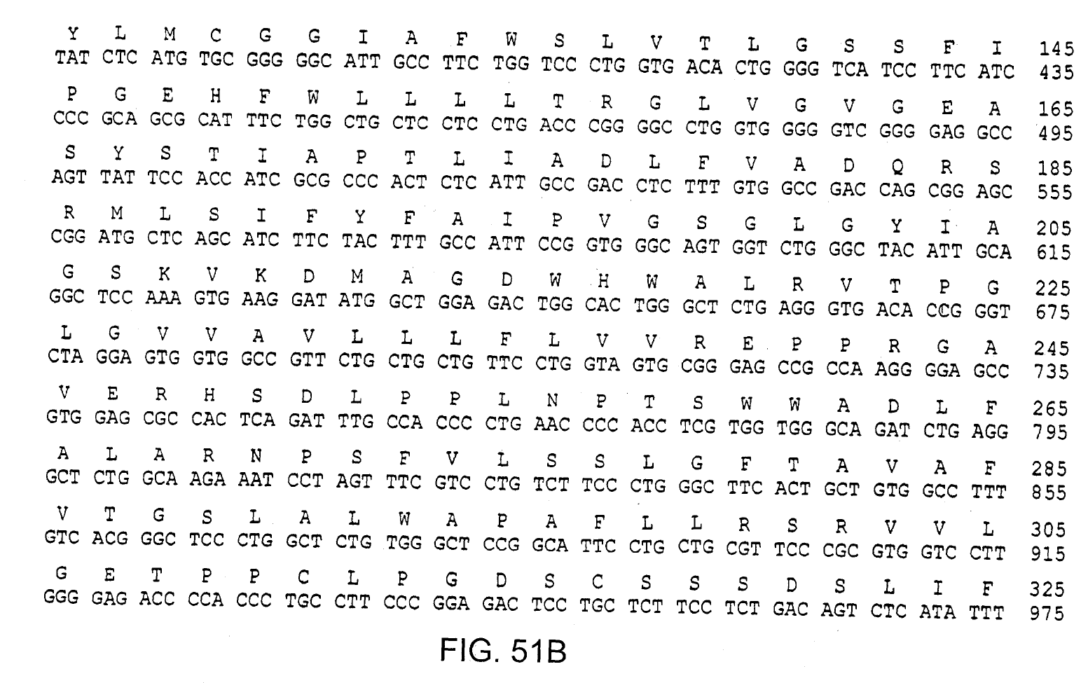

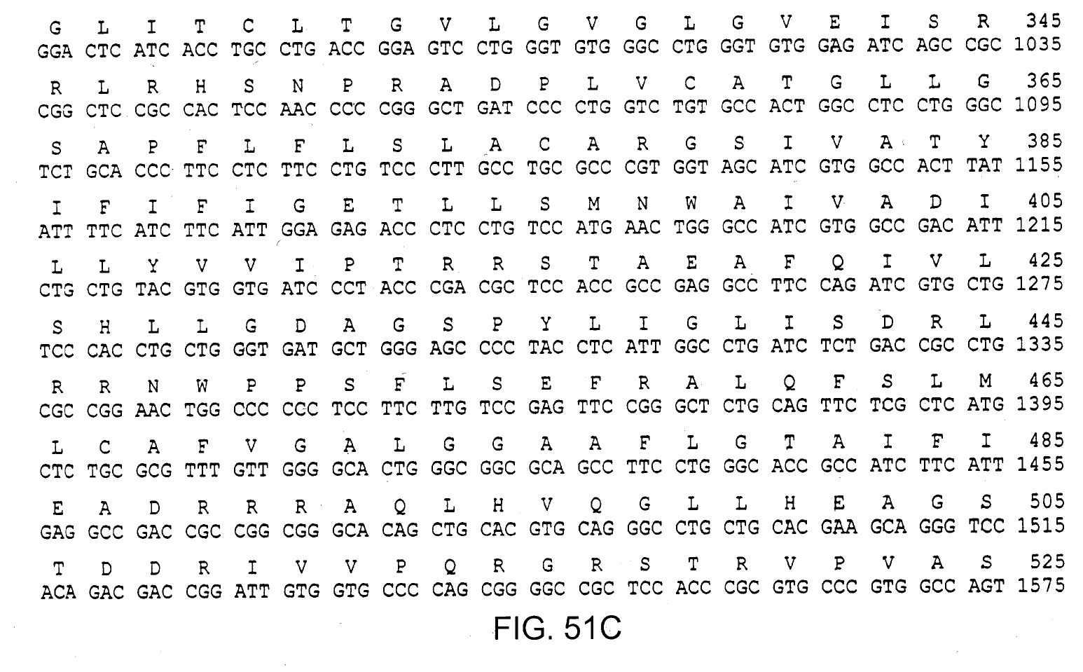

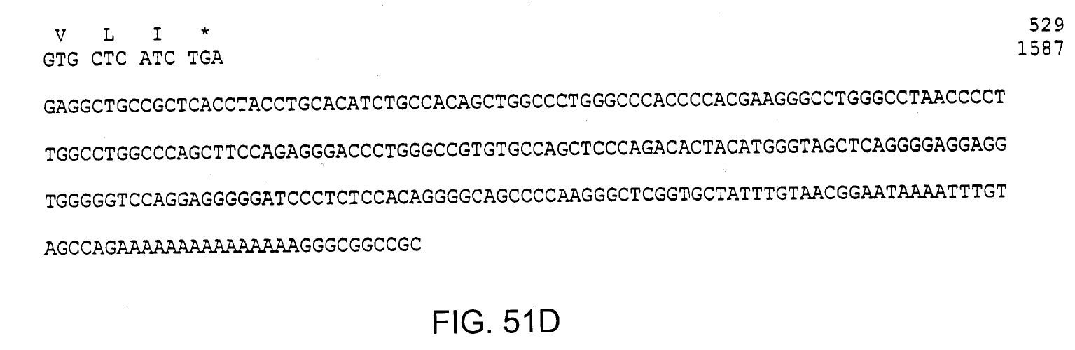

The invention is further based, at least in part, on the discovery of a human gene encoding a novel polypeptide, referred to herein as human 46455. The entire sequence of the human clone 46455 was determined and found to contain an open reading frame termed human “46455.” The nucleotide sequence of the human 46455 gene is set forth in FIGS. 47A-B and in the Sequence Listing as SEQ ID NO:49. The amino acid sequence of the human 46455 expression product is set forth in FIGS. 51A-51D and in the Sequence Listing as SEQ ID NO:50. The 46455 polypeptide comprises 528 amino acids. The coding region (open reading frame) of SEQ ID NO:49 is set forth as SEQ ID NO:51. Clone 46455, comprising the coding region of human 46455, was deposited with the American Type Culture Collection (ATCC®), 10801 University Boulevard, Manassas, Va. 20110-2209, on ______, and assigned Accession No. ______.

Analysis of the Human 8099 and 46455 Molecules

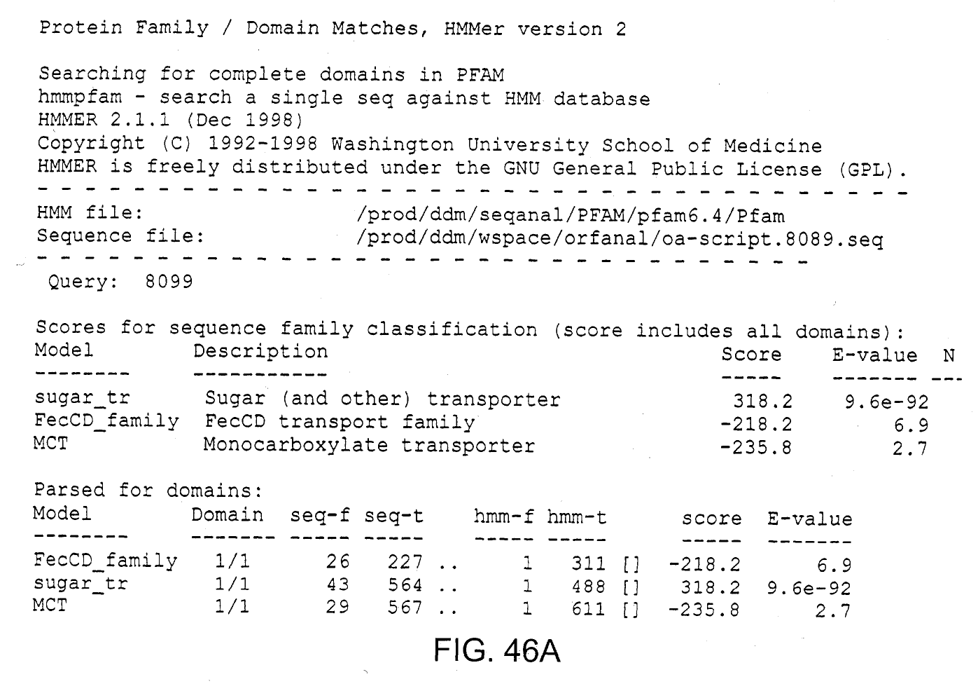

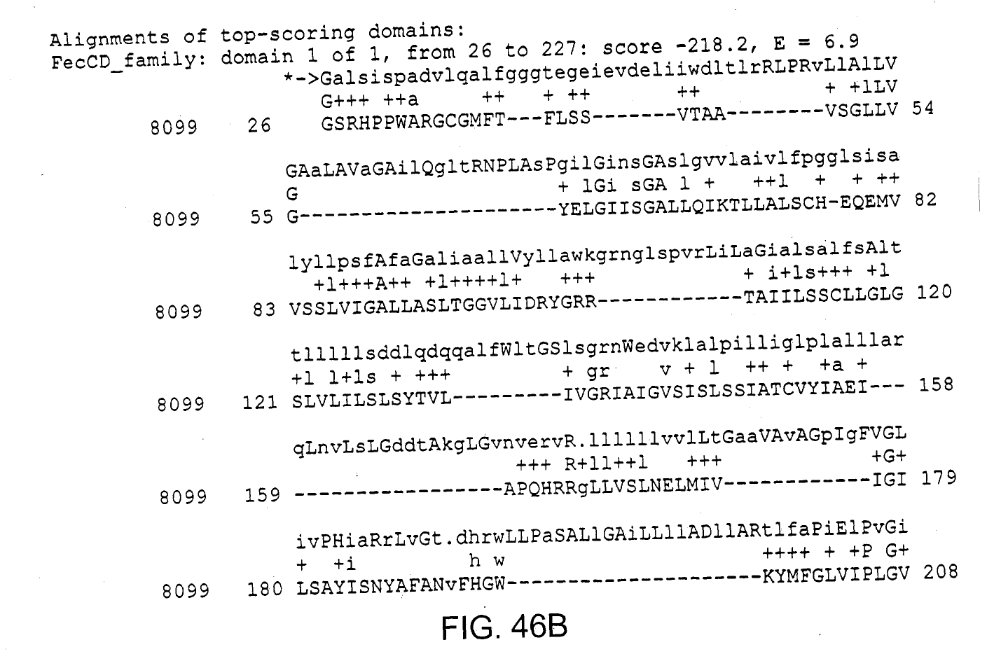

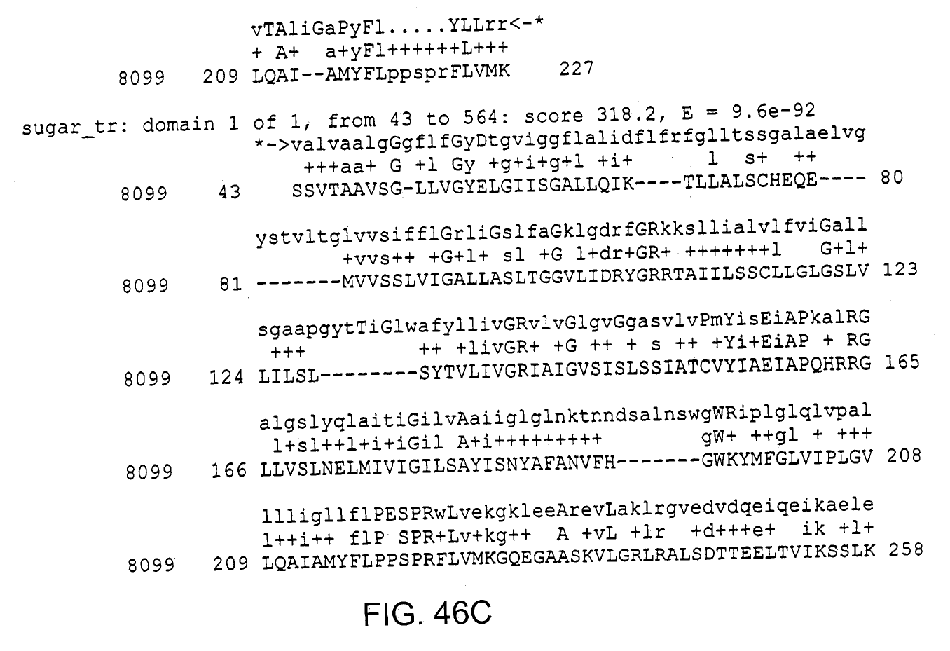

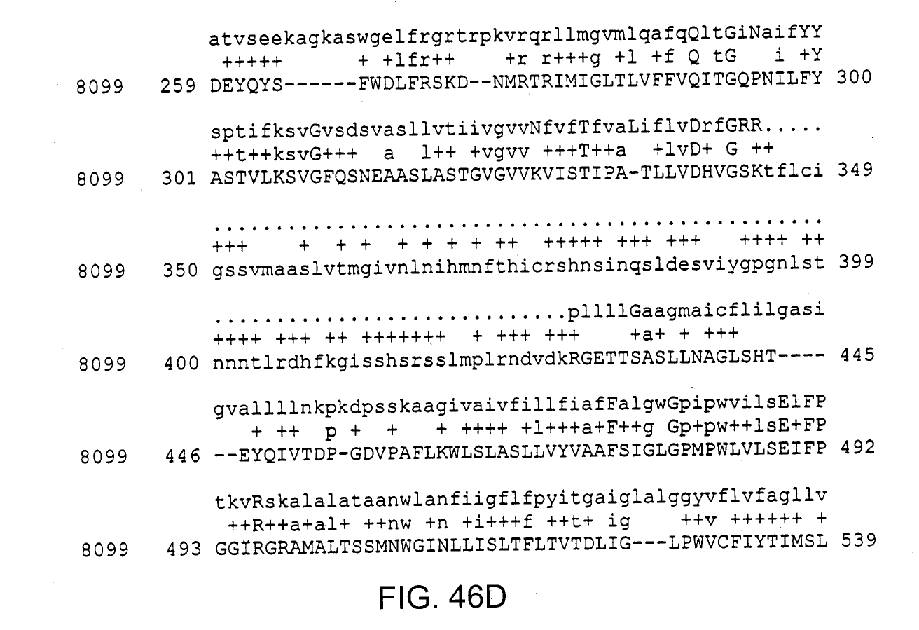

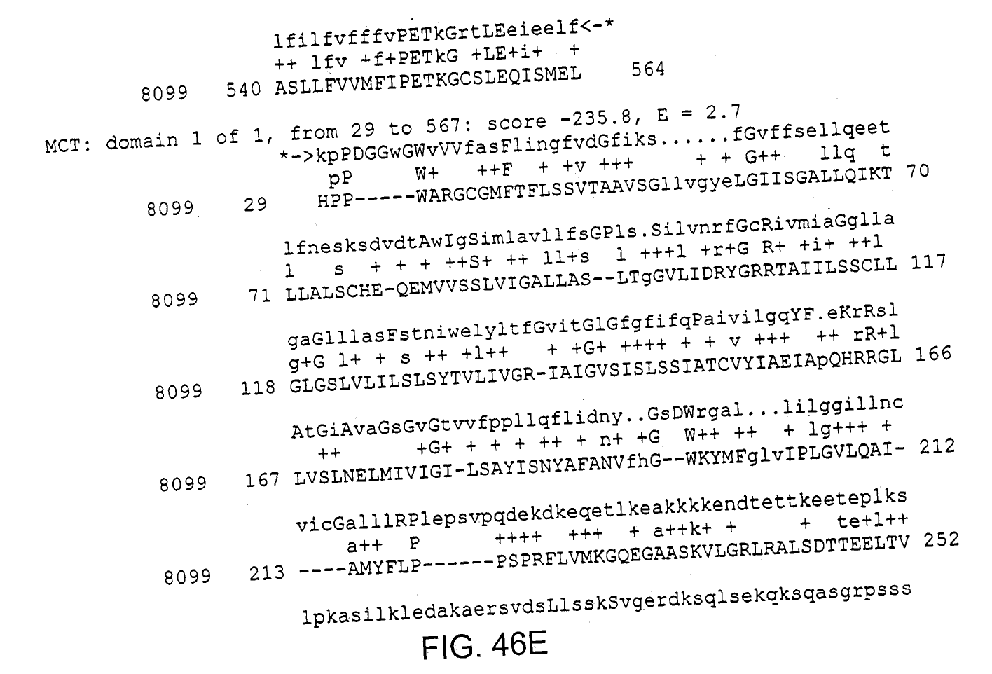

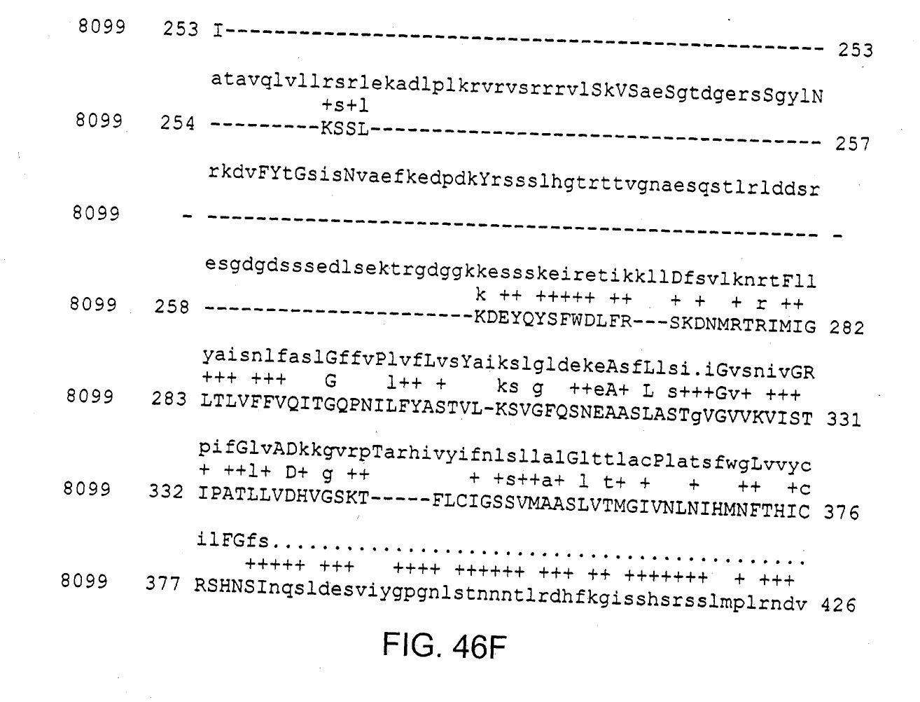

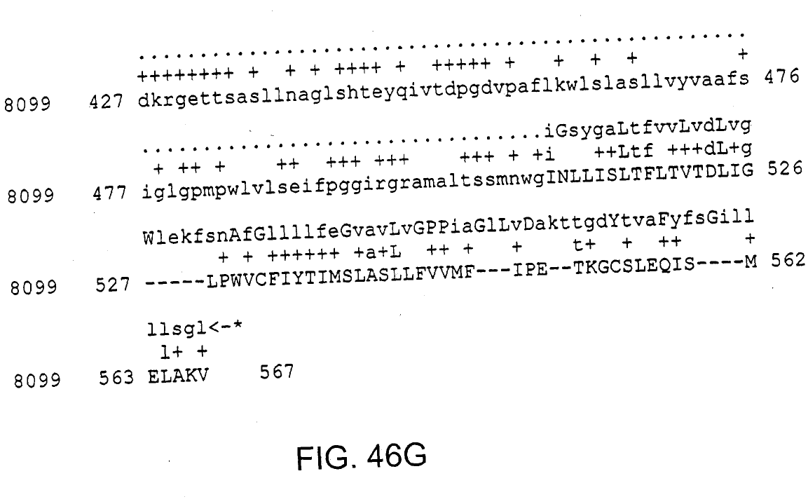

A search using the polypeptide sequence of SEQ ID NO:47 was performed against the HMM database in PFAM (FIGS. 46A-46G) resulting in the identification of a sugar transporter family domain in the amino acid sequence of human 8099 at about residues 43-564 of SEQ ID NO:47 (score=318.2), a potential FecCD family domain in the amino acid sequence of human 8099 at about residues 26-227 of SEQ ID NO:47 (score=−218.2), and a potential monocarboxylate transporter domain in the amino acid sequence of human 8099 at about residues 29-567 of SEQ ID NO:47 (score=−235.8).

The amino acid sequence of human 8099 was analyzed using the program PSORT (available through the Prosite website) to predict the localization of the proteins within the cell. This program assesses the presence of different targeting and localization amino acid sequences within the query sequence. The results of this analysis show that human 8099 may be localized to the endoplasmic reticulum or mitochondria.

Searches of the amino acid sequence of human 8099 were further performed against the Prosite database. These searches resulted in the identification in the amino acid sequence of human 8099 of a number of potential N-glycosylation sites at about amino acid residues 371-374, 383-386, 396-399, 401-404 of SEQ ID NO:47, a number of potential protein kinase C phosphorylation sites at about amino acid residues 220-222, 256-258, 403-405 of SEQ ID NO:47, a number of potential casein kinase II phosphorylation sites at about amino acid residues 18-21, 75-78, 169-172, 246-249, 256-259, 264-267, 385-388, 403-406, 443-446, 520-523 of SEQ ID NO:47, a number of potential N-myristoylation sites at about amino acid residues 51-56, 59-64, 89-94, 141-146, 165-170, 178-183, 207-212, 228-233, 395-400, 441-446, and 493-498 of SEQ ID NO:47, a potential amidation site at about amino acid residues 104-107 of SEQ ID NO:47, a potential leucine zipper motif at about amino acid residues 112-133 of SEQ ID NO:47, and potential sugar transport protein signature 1 domain at about amino acid residues 97-114 of SEQ ID NO:47.

A MEMSAT analysis of the polypeptide sequence of SEQ ID NO:47 was also performed, predicting thirteen transmembrane domains in the amino acid sequence of human 8099 (SEQ ID NO:47) at about residues 32-49, 58-74, 81-101, 109-130, 138-156, 165-184, 198-217, 279-301, 315-338, 346-364, 463-487, 499-521, and 529-549. Further analysis of the amino acid sequence of SEQ ID NO:47 (e.g., alignment with, for example, known E. coli sugar symporter proteins and a known human facilitative glucose transporter protein) showed that the second transmembrane domain at about amino acid residues 58-74 of SEQ ID NO:47 is not utilized, resulting in the presence of twelve transmembrane domains in the amino acid sequence of human 8099 (SEQ ID NO:47).

A search of the amino acid sequence of human 8099 was also performed against the ProDom database resulting in the identification of several transmembrane domains, a glycosyltransferase domain, and a sugar transport domain in the amino acid sequence of SEQ ID NO:47.

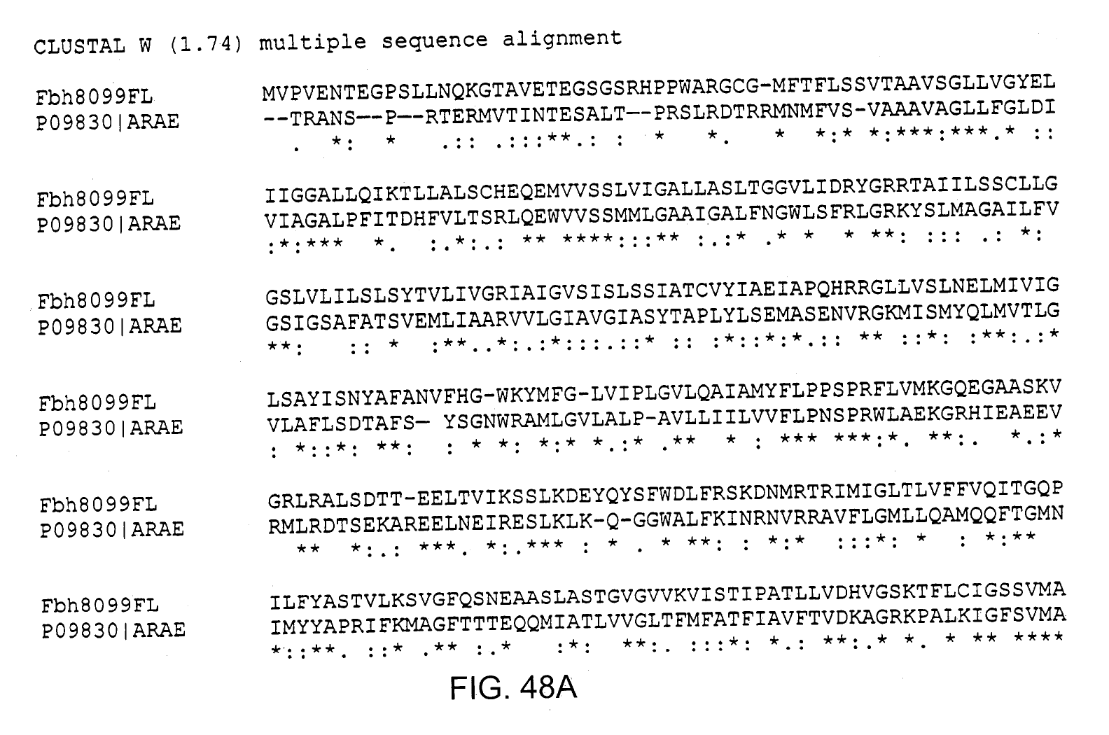

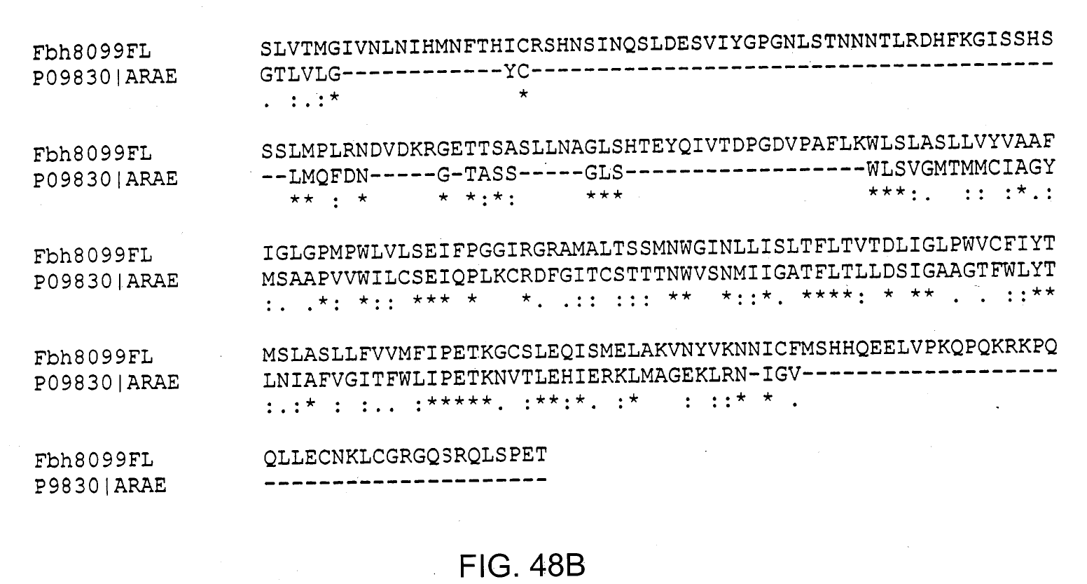

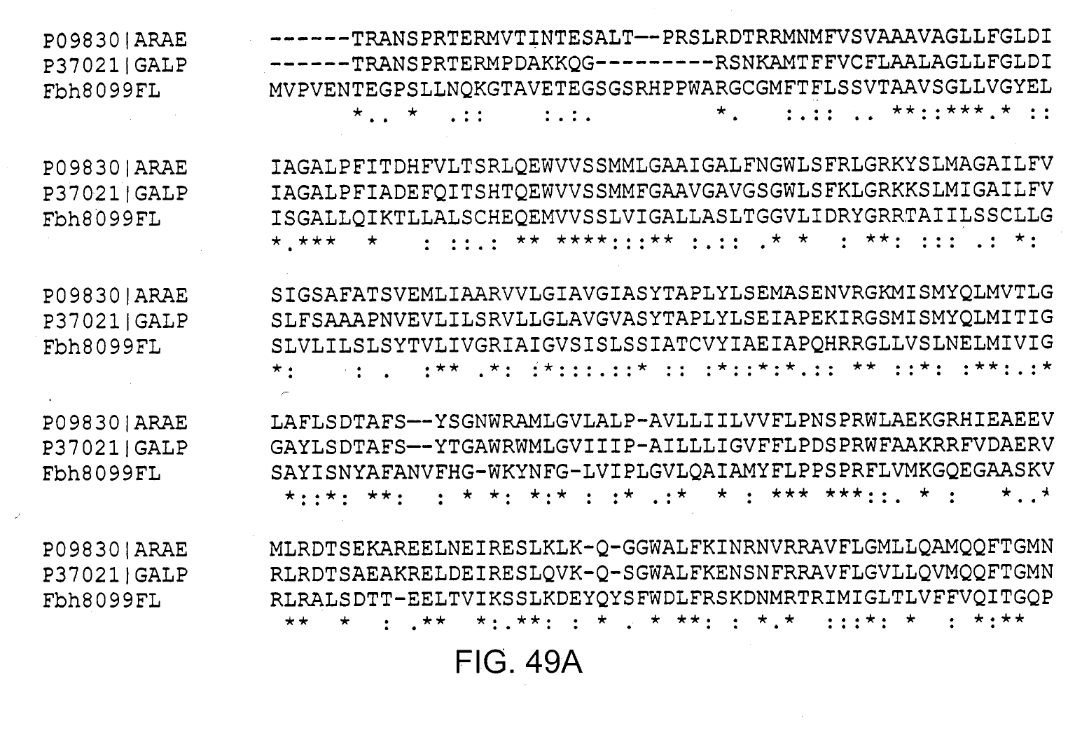

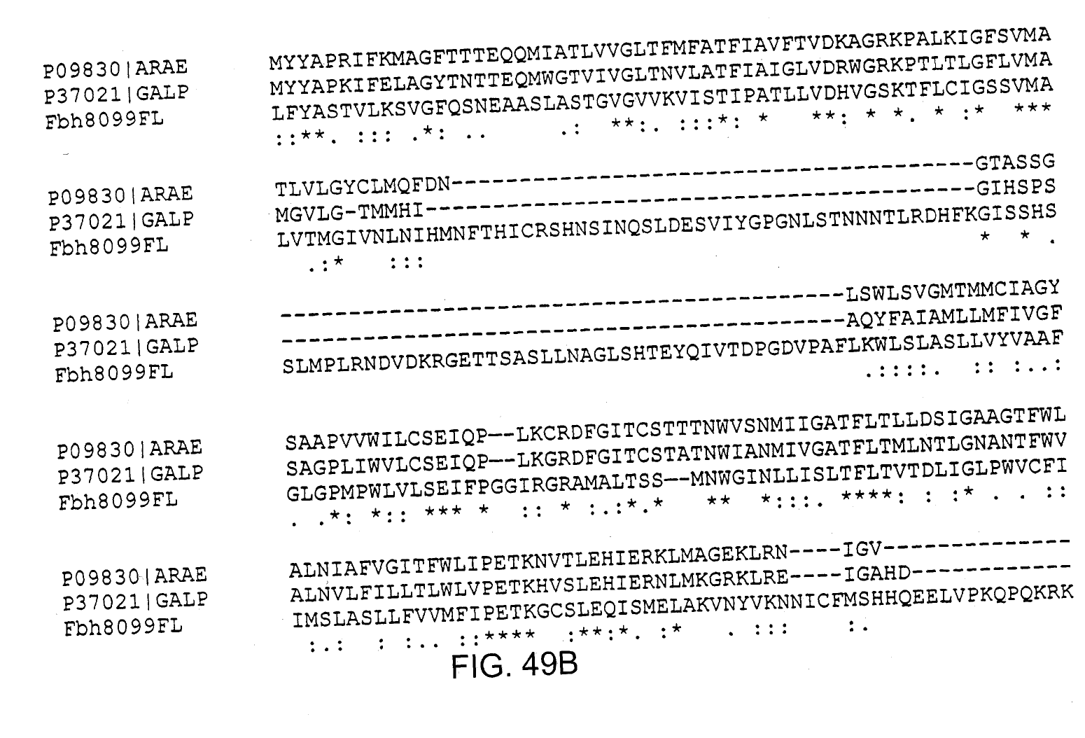

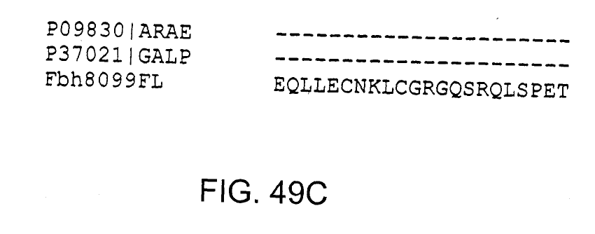

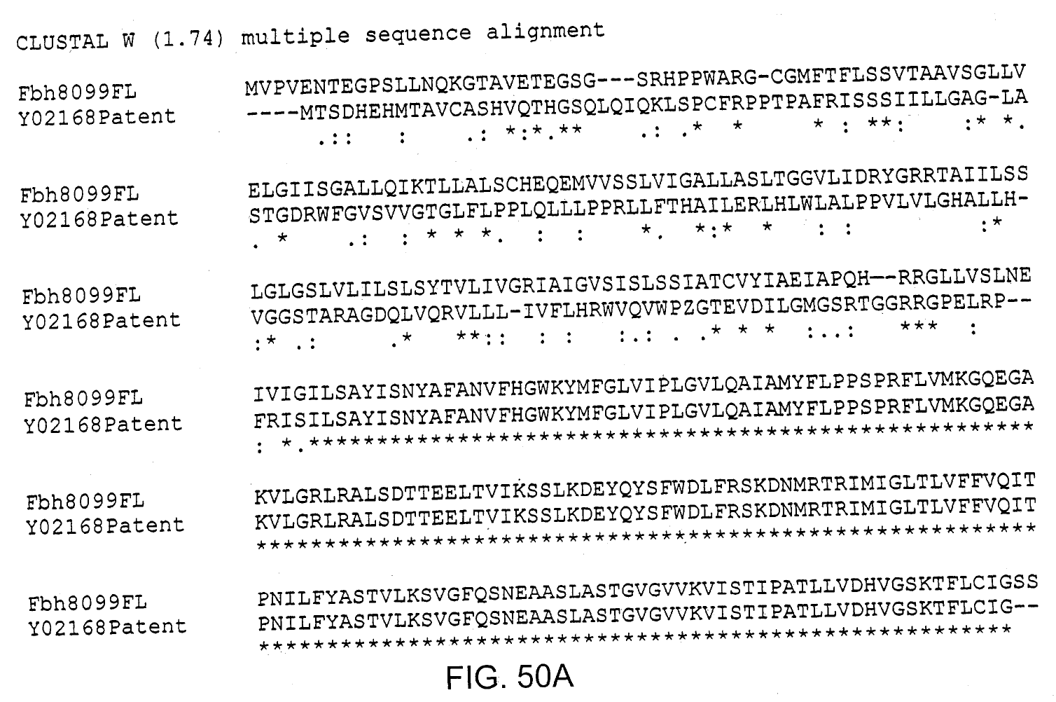

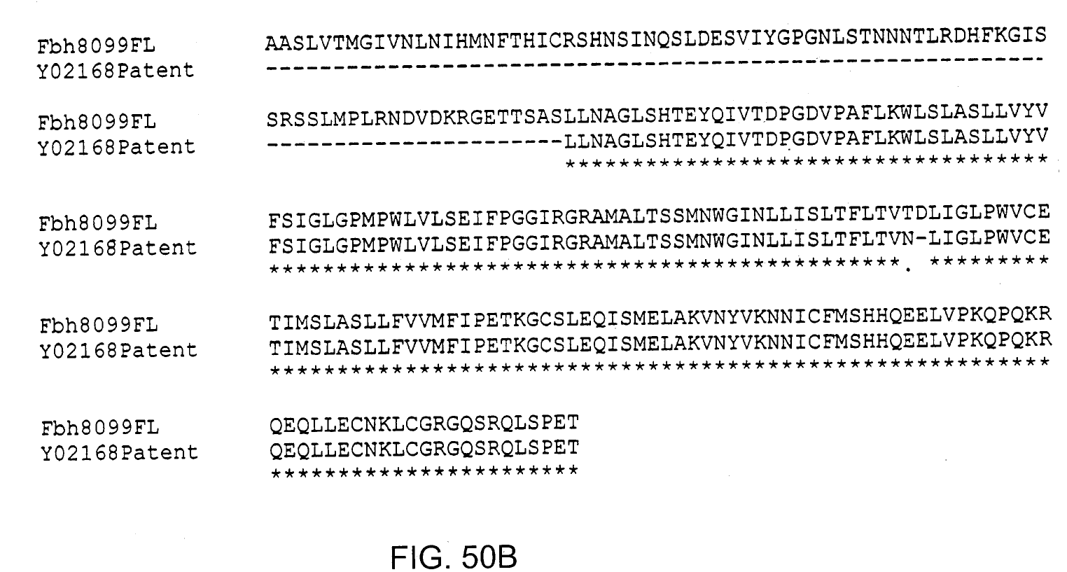

The human 8099 amino acid sequence was aligned with the amino acid sequence of the galactose-proton symporter GALP from E. coli l using the CLUSTAL W (1.74) multiple sequence alignment program. The results of the alignment are set forth in FIGS. 47A-B. The human 8099 amino acid sequence was also aligned with the amino acid sequence of the arabinose-proton symporter ARAE from E. coli using the CLUSTAL W (1.74) multiple sequence alignment program. The results of the alignment are set forth in FIGS. 48A-B. The human 8099 amino acid sequence was also aligned with the amino acid sequence of the facilitative glucose transporter GLUT8 from Homo sapiens using the CLUSTAL W (1.74) multiple sequence alignment program. The results of the alignment are set forth in FIGS. 50A-B. Based on its homology to GLUT8, 8099 is also referred to herein as “GLUT8 homologue” or “GLUT8h” and can be used interchangeably throughout.

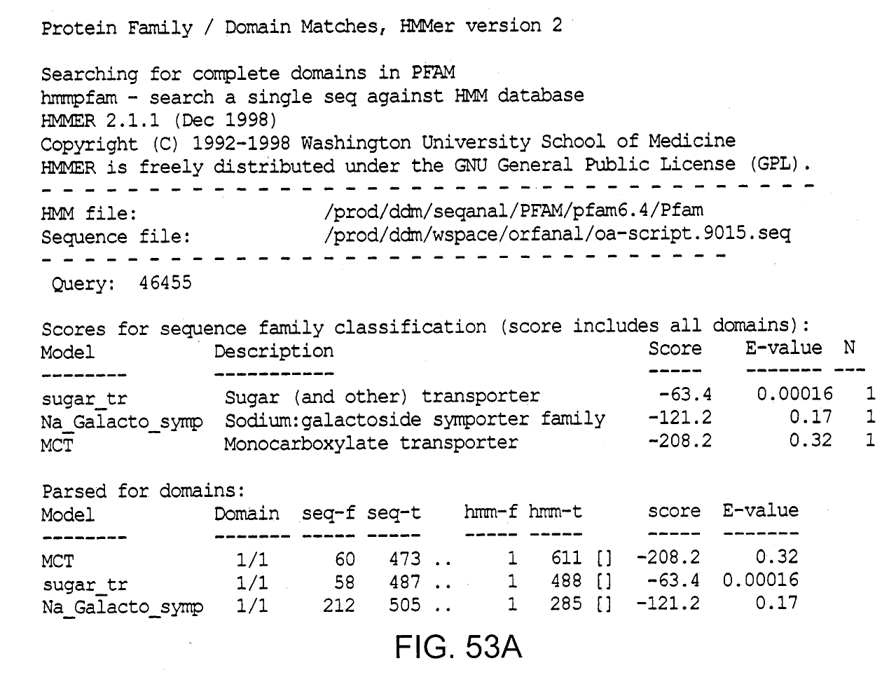

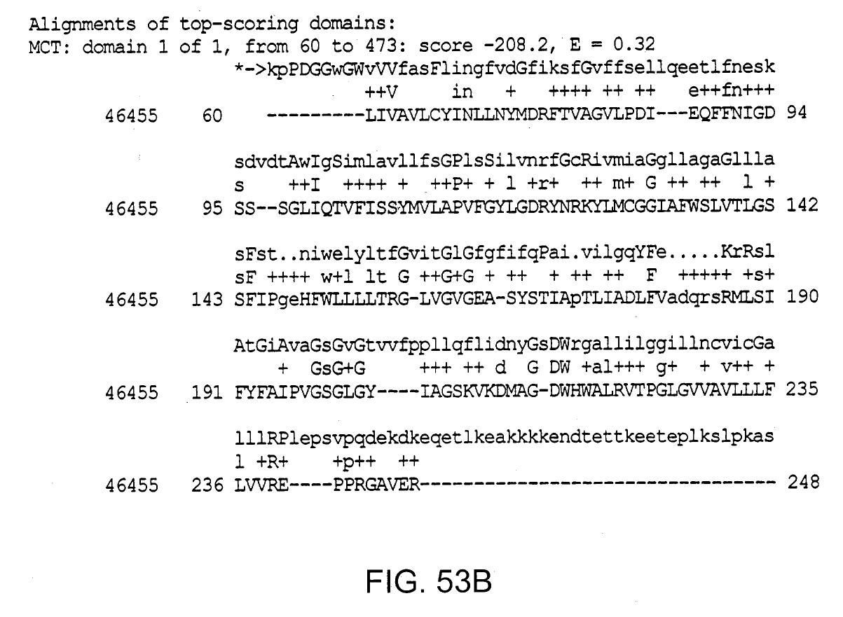

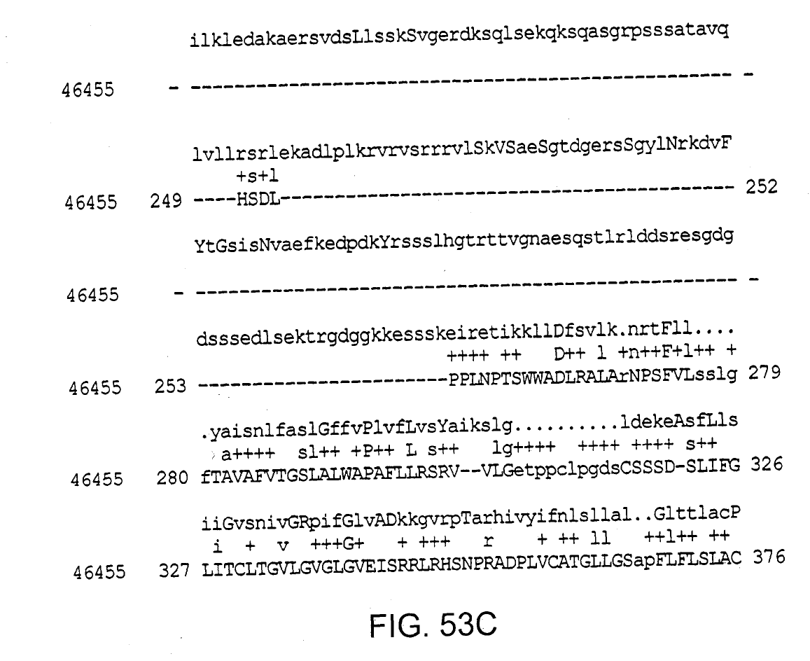

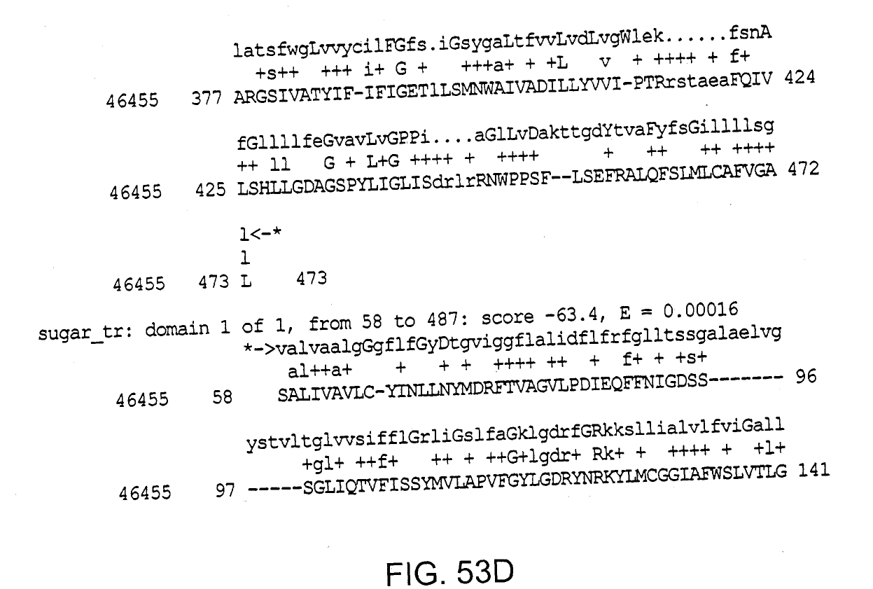

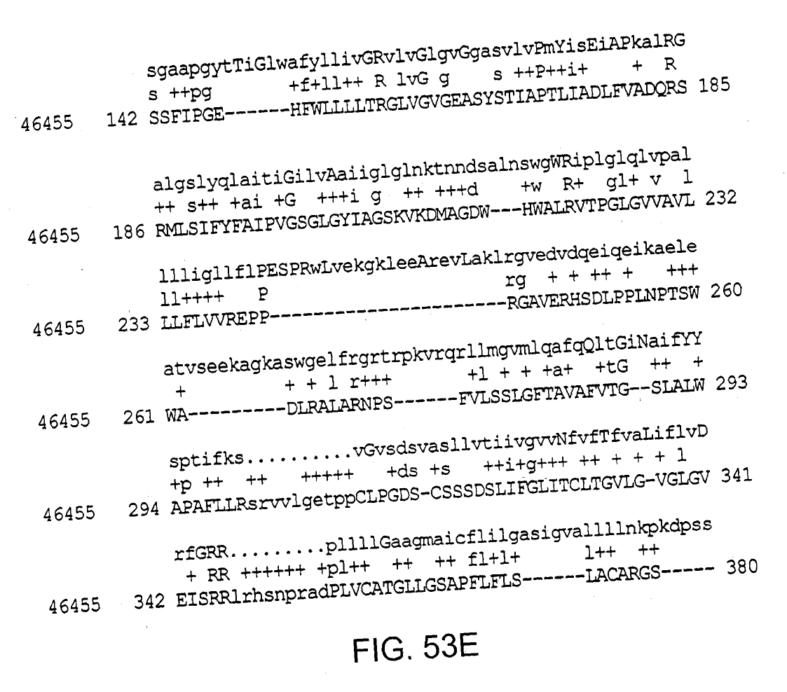

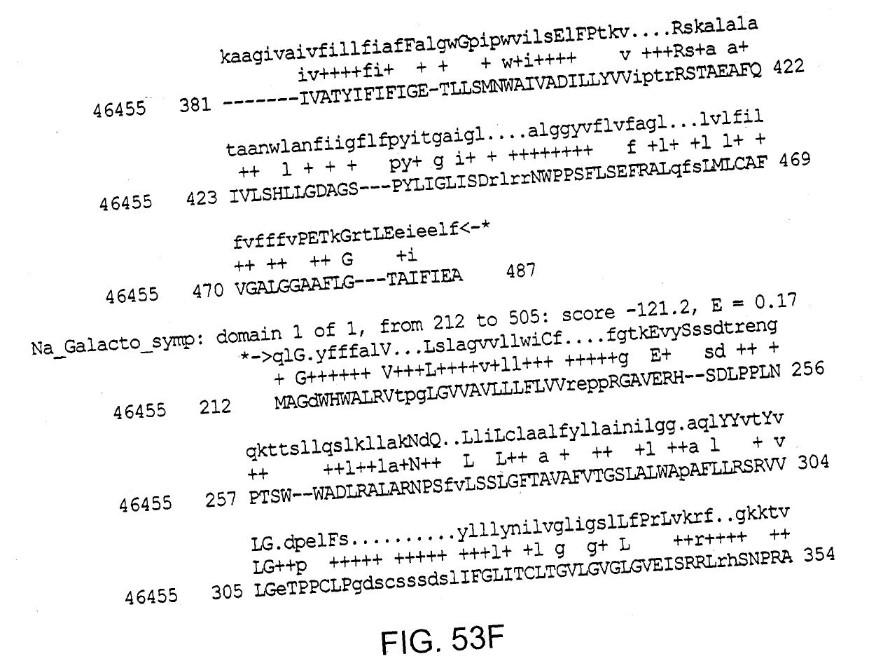

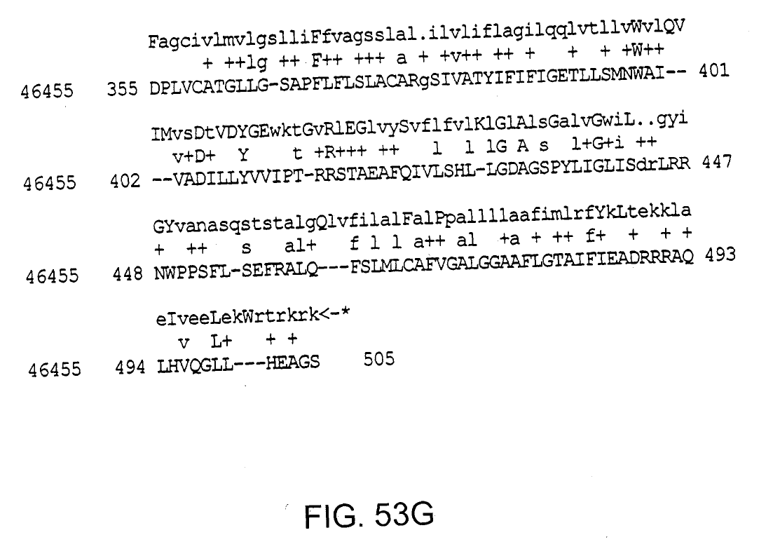

A search using the polypeptide sequence of human 46455 (SEQ ID NO:50) was performed against the HMM database in PFAM (FIGS. 53A-53G) resulting in the identification of a sugar transporter family domain in the amino acid sequence of human 46455 at about residues 58-469 of SEQ ID NO:50 (score=−63.4), a potential sodium:galactoside symporter family domain in the amino acid sequence of human 46455 at about residues 212-505 of SEQ ID NO:50 (score=−121.2), and a potential monocarboxylate transporter domain in the amino acid sequence of human 46455 at about residues 60-473 of SEQ ID NO:50 (score=−208.2).

The amino acid sequence of human 46455 was analyzed using the program PSORT to predict the localization of the proteins within the cell. This program assesses the presence of different targeting and localization amino acid sequences within the query sequence. The results of this analysis show that human 46455 may be localized to the endoplasmic reticulum, mitochondria, nucleus or secretory vesicles.

Searches of the amino acid sequence of human 46455 were further performed against the Prosite database. These searches resulted in the identification in the amino acid sequence of human 46455 of a potential N-glycosylation site at about amino acid residues 199-202 of SEQ ID NO:50, a potential cAMP- and cGMP-dependent protein kinase C phosphorylation site at about amino acid residues 414-417 of SEQ ID NO:50, a number of potential protein kinase C phosphorylation sites at about amino acid residues 344-346, 413-415, 442-444, and 518-520 of SEQ ID NO:50, a number of potential casein kinase II phosphorylation sites at about amino acid residues 11-14, 943-946, 959-962, 983-986, 1074-1077, 1108-1111, and 1112-1115 of SEQ ID NO:50, a number of potential N-myristoylation sites at about amino acid residues 89-94, 106-111, 288-293, 679-684, 767-772, 847-852, and 933-938 of SEQ ID NO:50, an amidation site at about amino acid residues 223-226 of SEQ ID NO:50, and an ATP/GTP-binding site motif A (P-loop) at about amino acid residues 1008-1015 of SEQ ID NO:50.

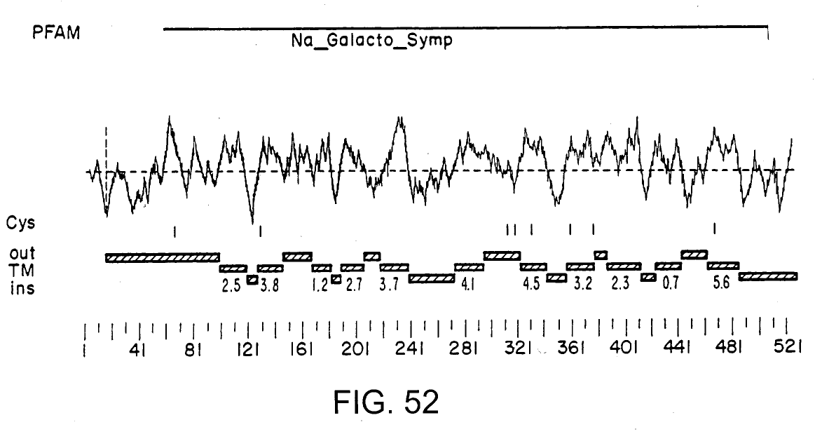

A MEMSAT analysis of the polypeptide sequence of SEQ ID NO:50 was also performed, predicting eleven transmembrane domains in the amino acid sequence of human 46455 (SEQ ID NO:50) at about residues 98-118, 126-145, 165-181, 188-205, 218-238, 273-294, 323-341, 357-377, 386-410, 423-441, and 462-485. Further analysis of the amino acid sequence of SEQ ID NO:50 (e.g., analysis of the hydropathy plot set forth in FIG. 52) resulted in the identification of a twelfth transmembrane domain at about amino acid residues 58-74 of SEQ ID NO:50.

A search of the amino acid sequence of human 46455 was also performed against the ProDom database resulting in the identification of a transmembrane efflux domain in the amino acid sequence of SEQ ID NO:50.

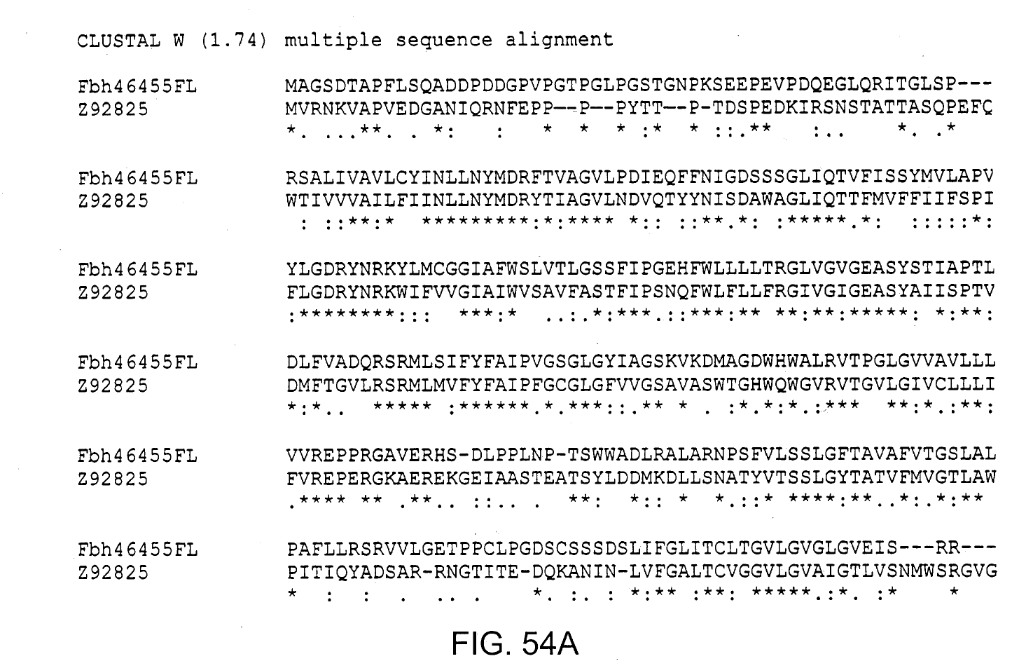

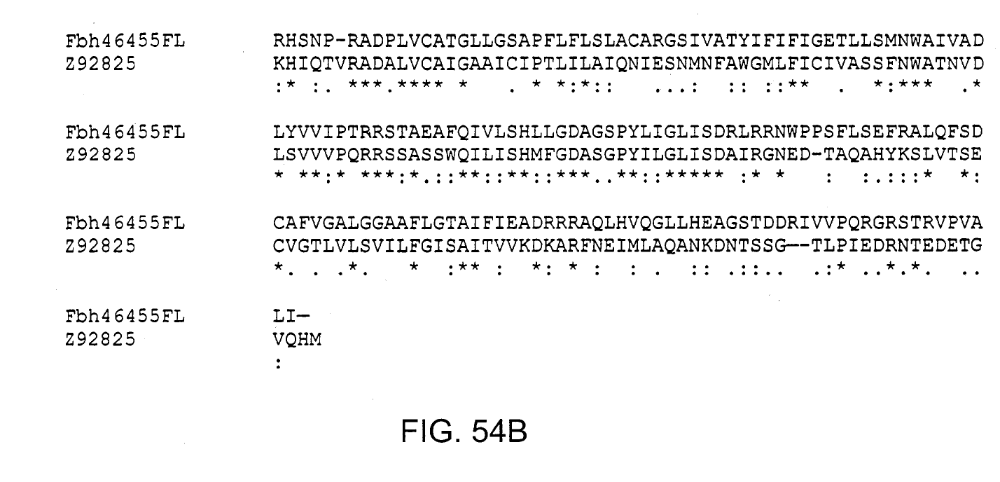

The human 46455 amino acid sequence was aligned with the amino acid sequence of Z92825 from C. elegans using the CLUSTAL W (1.74) multiple sequence alignment program. The results of the alignment are set forth in FIGS. 54A-B.

Isolation of the Human 54414 and 53763 cDNA

The invention is based, at least in part, on the discovery of genes encoding novel members of the potassium channel family. The entire sequence of human clone Fbh54414 was determined and found to contain an open reading frame termed human “54414”. The entire sequence of human clone Fbh53763 was determined and found to contain an open reading frame termed human “53763”.

The nucleotide sequence encoding the human 54414 is shown in FIGS. 55A-55H and is set forth as SEQ ID NO:52. The protein encoded by this nucleic acid comprises about 1118 amino acids and has the amino acid sequence shown in FIGS. 55A-55H and set forth as SEQ ID NO:53. The coding region (open reading frame) of SEQ ID NO:52 is set forth as SEQ ID NO:54. Clone Fbh54414, comprising the coding region of human 54414, was deposited with the American Type Culture Collection (ATCC®), 10801 University Boulevard, Manassas, Va. 20110-2209, on ______, and assigned Accession No. ______.

The nucleotide sequence encoding the human 53763 is shown in FIGS. 59A-59D and is set forth as SEQ ID NO:55. The protein encoded by this nucleic acid comprises about 638 amino acids and has the amino acid sequence shown in FIGS. 59A-59D and set forth as SEQ ID NO:56. The coding region (open reading frame) of SEQ ID NO:55 is set forth as SEQ ID NO:57. Clone Fbh53763, comprising the coding region of human 53763, was deposited with the American Type Culture Collection (ATCC®), 10801 University Boulevard, Manassas, Va. 20110-2209, on ______, and assigned Accession No. ______.

Analysis of the Human 54414 and 53763 Molecules

The amino acid sequences of human 54414 was analyzed using the program PSORT to predict the localization of the proteins within the cell. The results of the analyses show that human 54414 may be localized to the endoplasmic reticulum, the nucleus, secretory vesicles, or the mitochondria.

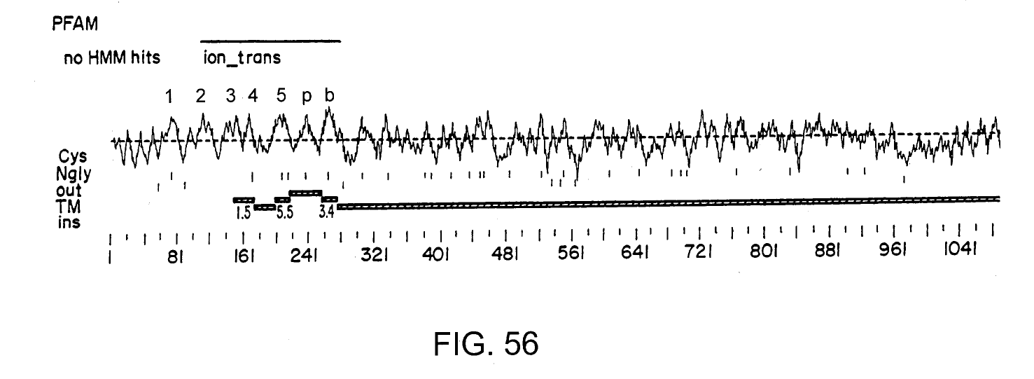

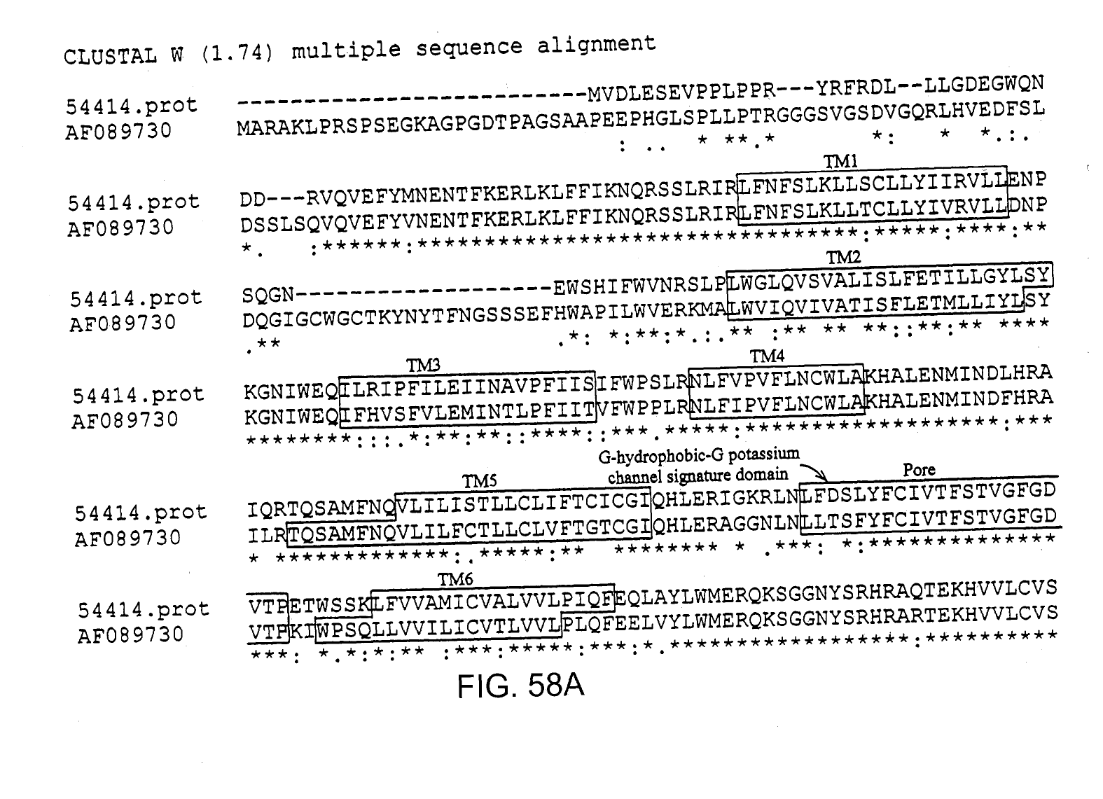

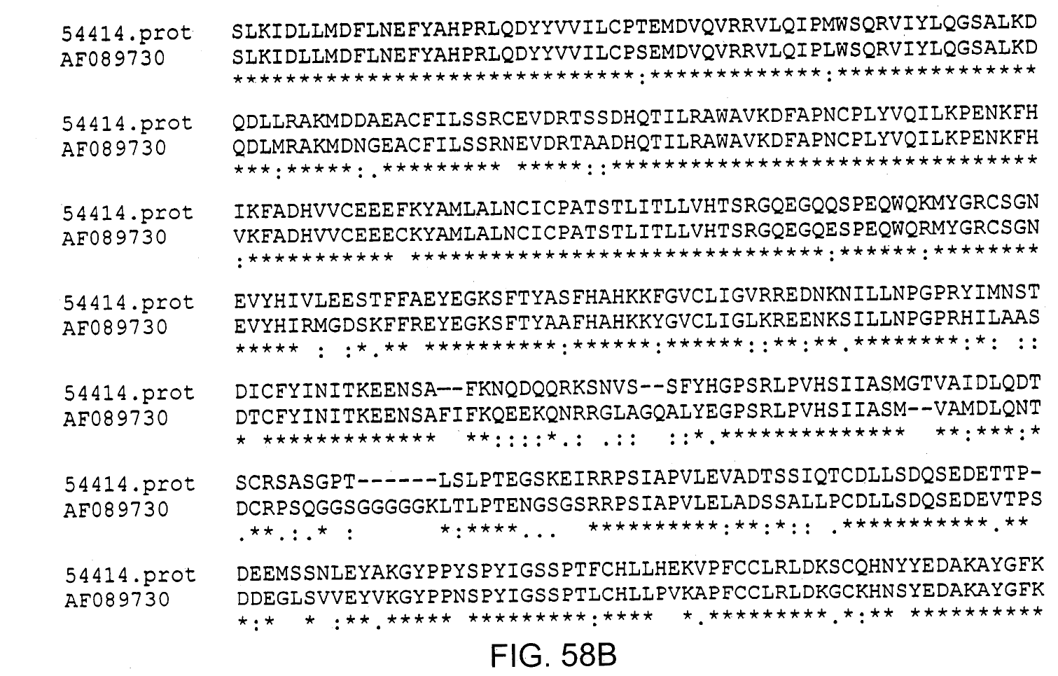

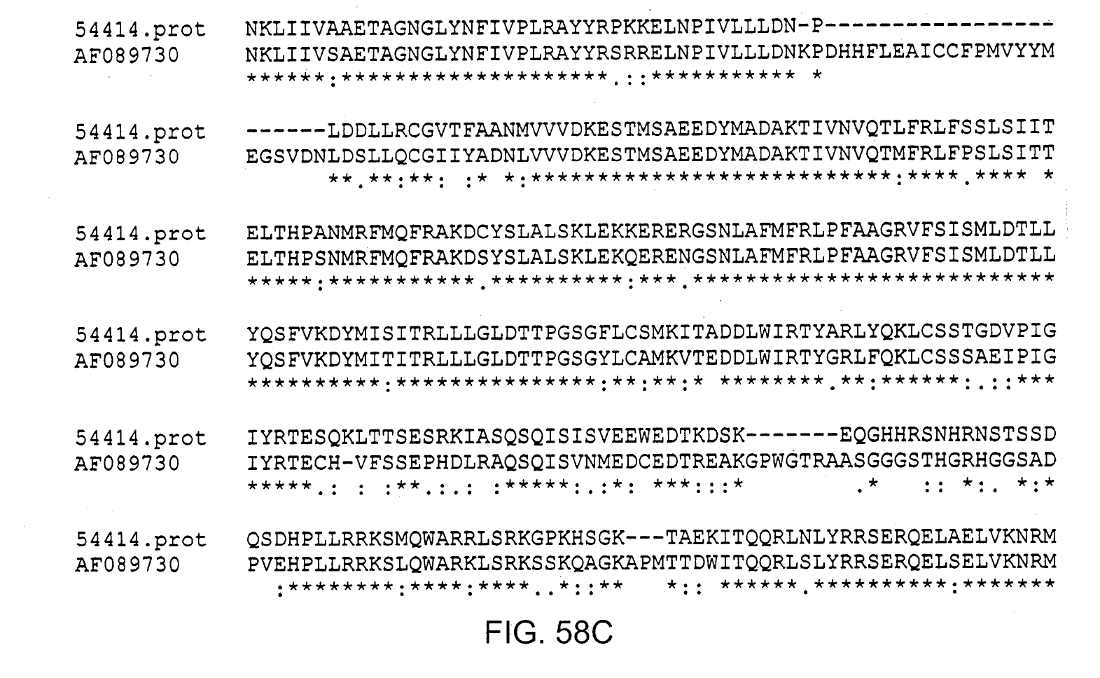

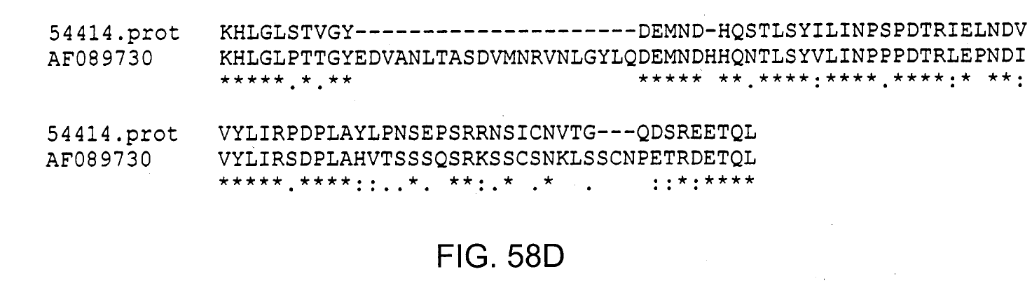

Analysis of the amino acid sequences of human 54414 was performed using MEMSAT. The amino acid sequence of human 54414 was also compared to the amino acid sequences of known potassium transporters (FIGS. 58A-58D). This analysis resulted in the identification of six possible transmembrane domains in the amino acid sequence of human 54414 at residues 64-83, 104-127, 135-153, 161-173, 199-217, and 257-274 of SEQ ID NO:53 (FIG. 56).

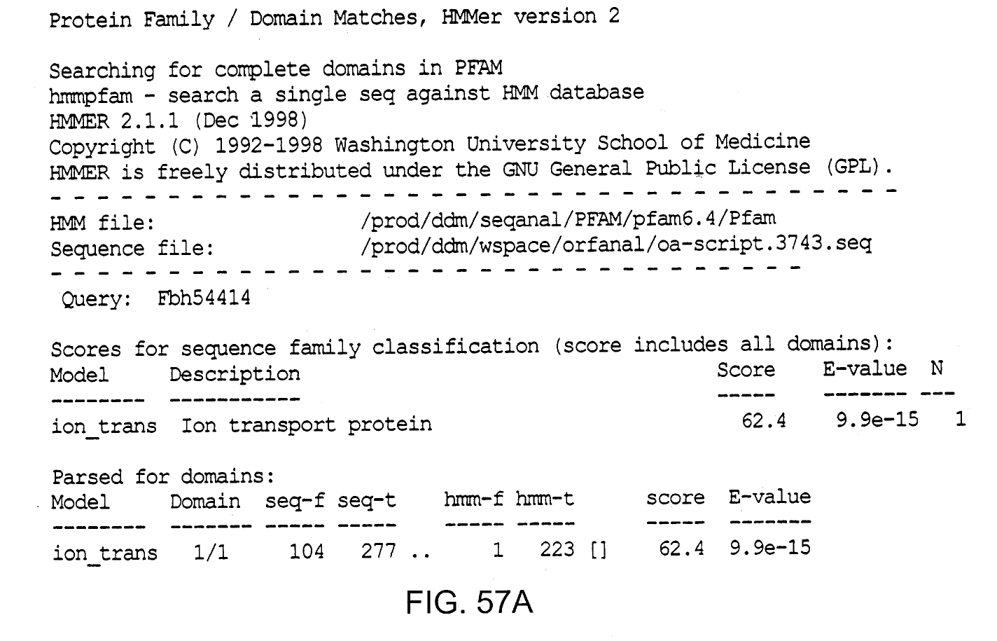

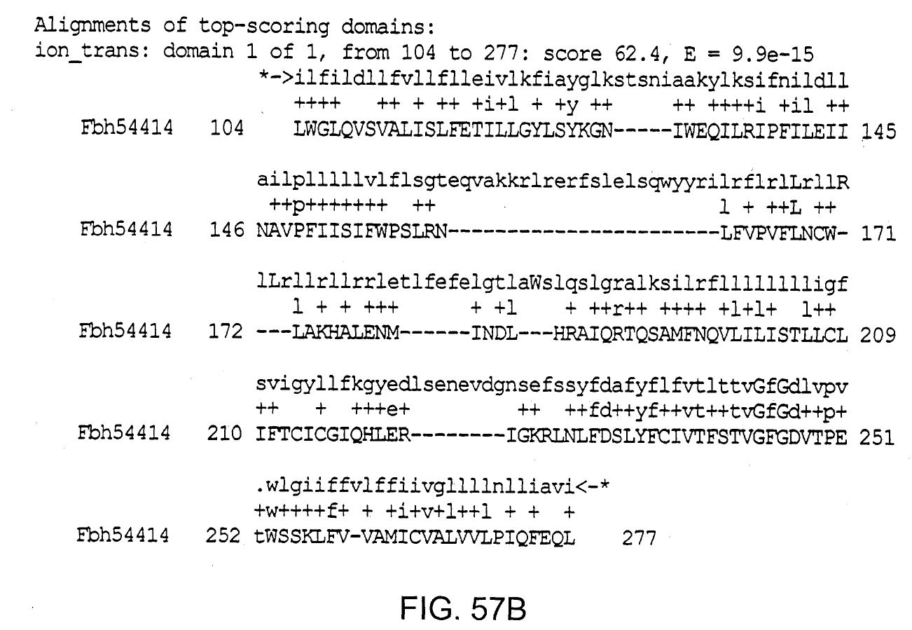

Searches of the amino acid sequences of human 54414 were performed against the HMM database (FIGS. 57A-B). These searches resulted in the identification of an “ion transport protein domain”, at about residues 104-277 of SEQ ID NO:53 (score=62.4).

Searches of the amino acid sequence of human 54414 were further performed against the Prosite™ database. These searches resulted in the identification of several possible N-glycosylation sites at about amino acids residues 66-69, 99-102, 290-293, 545-548, 554-557, 573-576, 981-984, and 1106-1109 of SEQ ID NO:53, several possible cAMP- and cGMP-dependent protein kinase phosphorylation sites at about amino acids residues 625-628, 994-997, 1002-1005, and 1100-1103 of SEQ ID NO:53, several possible protein kinase C phosphorylation sites at about amino acid residues 43-45, 59-61, 68-70, 126-128, 158-160, 254-256, 298-300, 308-310, 354-356, 385-387, 464-466, 605-607, 903-905, 939-941, 947-949, 1005-1007, 1012-1014, 1030-1032, and 1099-1101 of SEQ ID NO:53, several possible casein kinase II phosphorylation sites at about amino acid residues 43-46, 115-118, 338-341, 386-389, 393-396, 485-488, 556-559, 651-654, 655-658, 663-666, 784-787, 837-840, 867-870, 907-910, 926-929, 943-946, 959-962, 983-986, 1074-1077, 1108-1111, and 1112-1115 of SEQ ID NO:53, several possible N-myristoylation sites at about amino acid residues 89-94, 106-111, 288-293, 679-684, 767-772, 847-852, and 933-938 of SEQ ID NO:53, a possible amidation site at about amino acid residues 223-226 of SEQ ID NO:53, and a possible ATP/GTP-binding site motif A (P-loop) at about amino acid residues 1008-1015 of SEQ ID NO:53.

The amino acid sequence of human 53763 was analyzed using the program PSORT to predict the localization of the proteins within the cell. The results of the analyses further show that human 53763 may be localized to the endoplasmic reticulum, the mitochondria, or the nucleus.

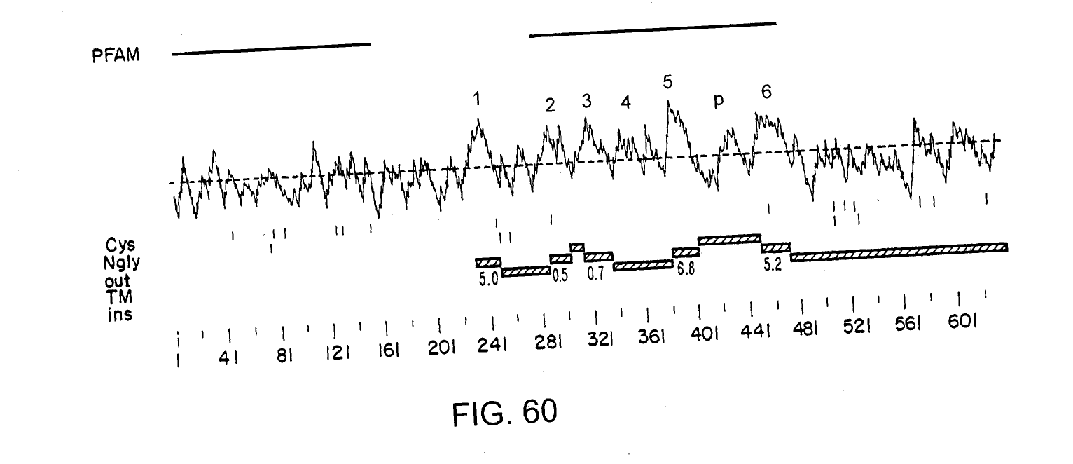

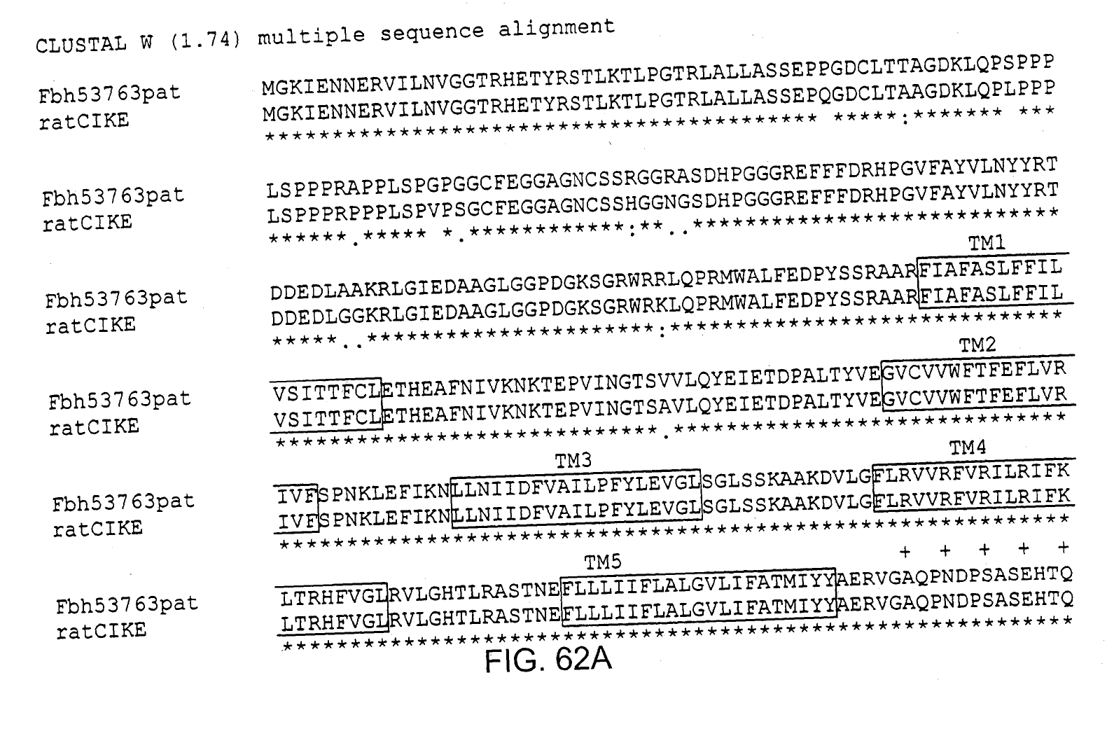

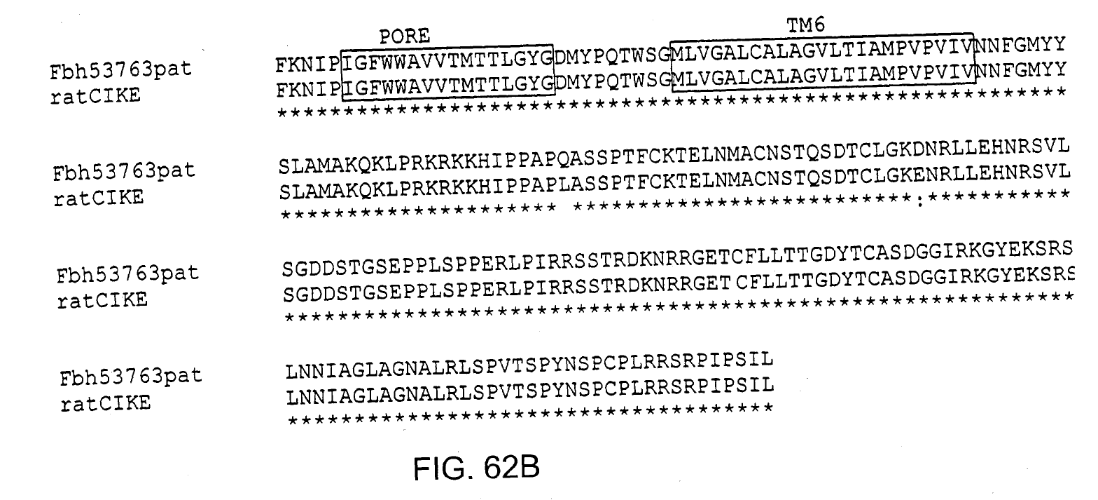

Analysis of the amino acid sequences of human 53763 was performed using MEMSAT. The amino acid sequence of human 53763 was also compared to the amino acid sequences of known potassium transporters (FIGS. 62A-B). This analysis resulted in the identification of six possible transmembrane domains in the amino acid sequence of human 53763 at residues 230-248, 287-303, 314-335, 346-368, 382-402, and 451-473 of SEQ ID NO:56 (FIG. 60).

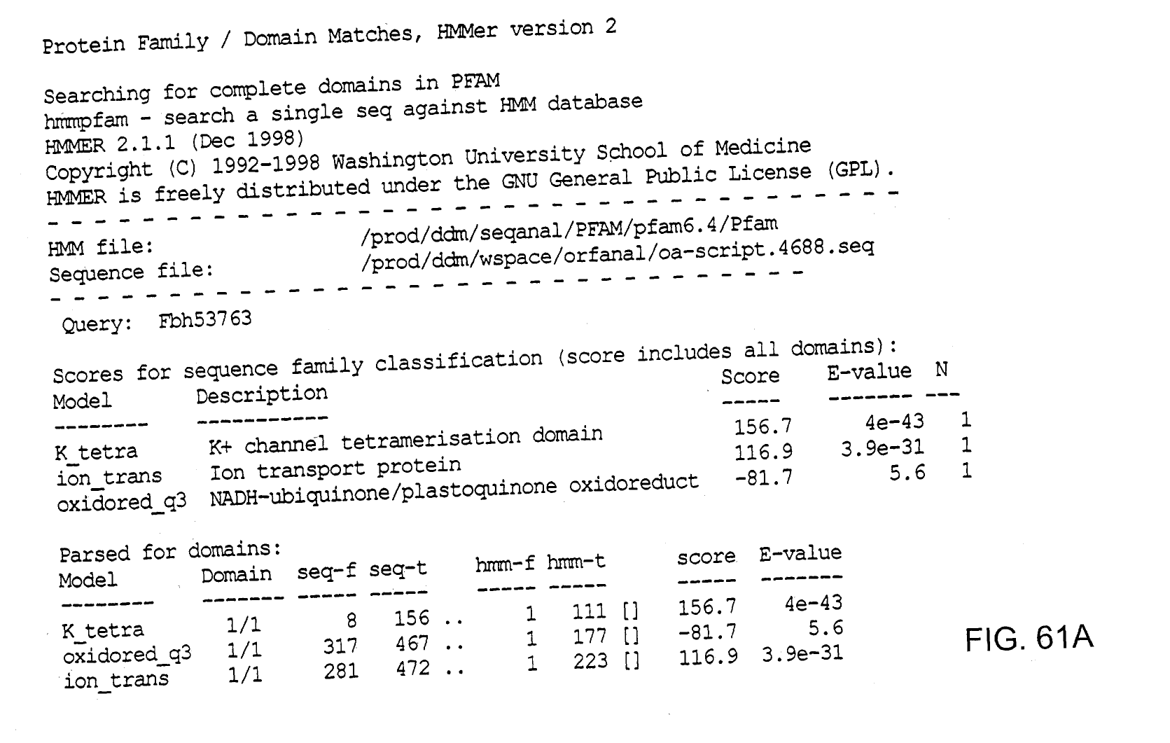

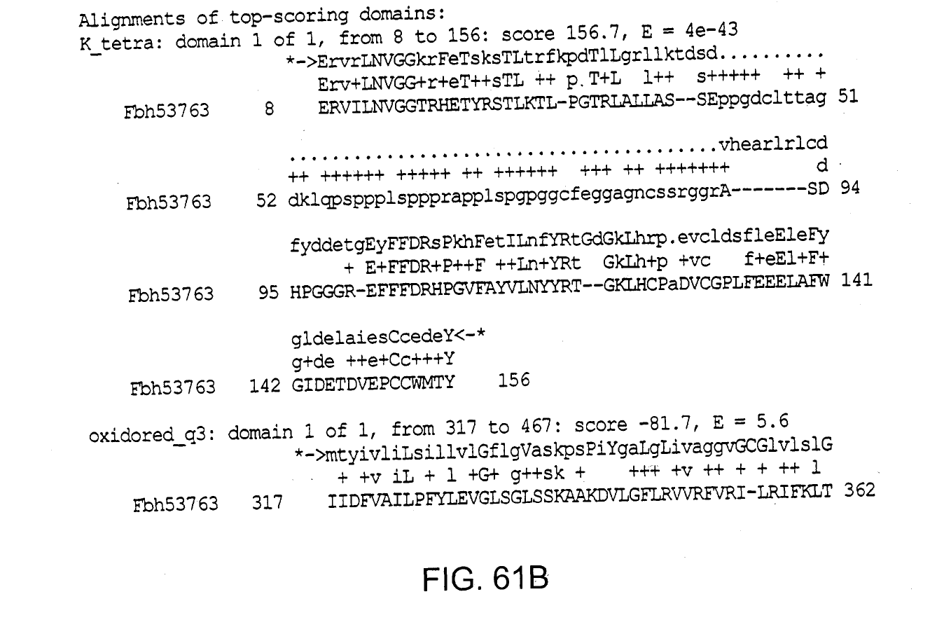

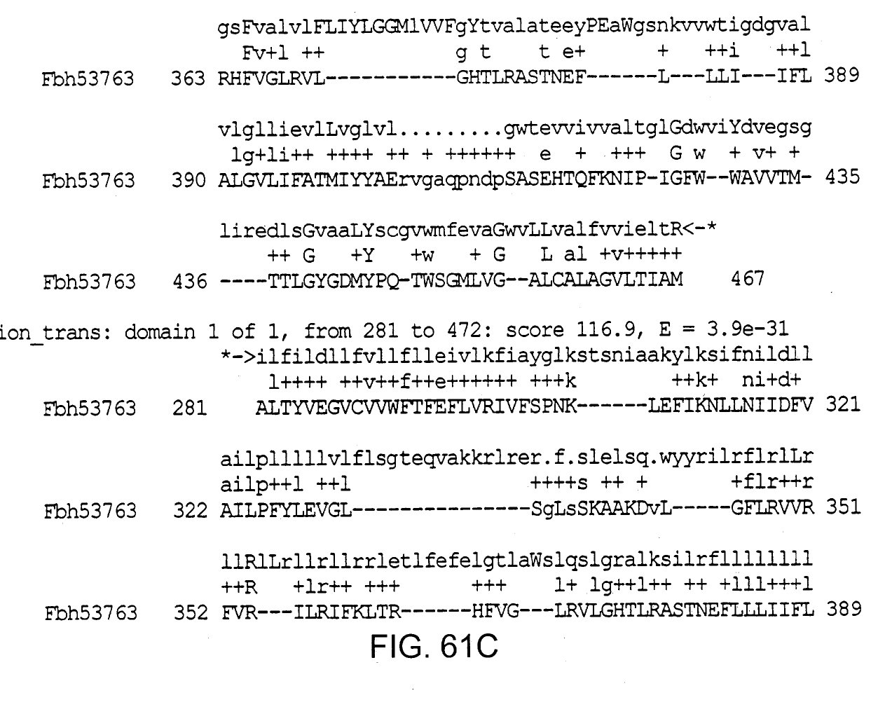

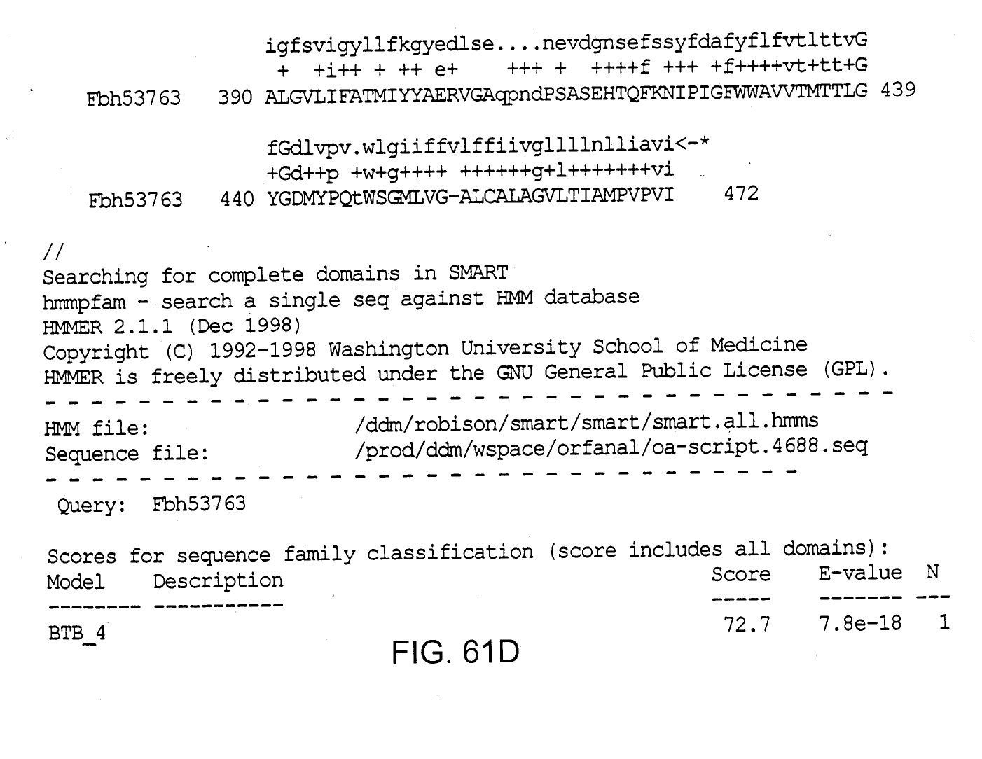

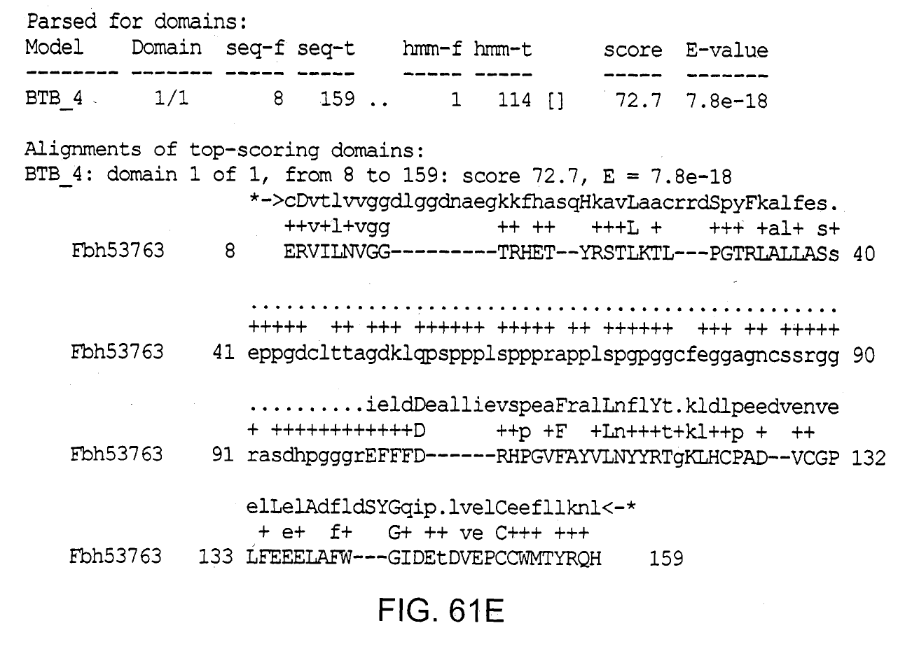

Searches of the amino acid sequence of human 53763 were also performed against the HMM database (FIGS. 61A-61E). These searches resulted in the identification of a “NADH-ubiquinone/plastoquinone oxidoreductase domain” at about residues 317-467 of SEQ ID NO:56 (score=−81.7), an “ion transport protein domain” at about residues 281-472 of SEQ ID NO:56 (score=116.9), and a “K⁺ channel tetramerization domain” at about residues 8-156 of SEQ ID NO:56 (score=156.7).

Searches of the amino acid sequence of human 53763 were also performed against the Prosite™ database. These searches resulted in the identification in the amino acid sequence of human 53763 a number of potential N-glycosylation sites at amino acid residues 84-84, 259-262, 266-269, 518-521, and 536-539 of SEQ ID NO:56, a potential cAMP- and cGMP-dependent protein kinase phosphorylation site at amino acid residues 561-564 of SEQ ID NO:56, protein kinase C phosphorylation sites at amino acid residues 21-23, 25-27, 86-88, 120-122, 155-157, 205-207, 224-226, 336-338, 374-376, and 564-566 of SEQ ID NO:56, casein kinase II phosphorylation sites at amino acid residues 17-20, 49-52, 146-149, 283-286, 378-381, 414-417, 520-523, 541-544, 546-549, 553-556, 564-567, and 579-582 of SEQ ID NO:56, and N-myristoylation sites at amino acid residues 31-36, 76-81, 83-88, 89-94, 142-147, 176-181, 191-196, 199-204, 407-412, 450-455, 477-482, 590-595, and 606-611 of SEQ ID NO:56.

Searches of the amino acid sequences of human 54414 and human 53763 were also performed against the ProDom database. A potassium ionic calcium activated domain and two potassium ionic subunits were identified in the amino acid sequence of 54414 (SEQ ID NO:53). Several transmembrane domains and transport family domains were identified in the amino acid sequence of 53763 (SEQ ID NO:56).

The amino acid sequences of human 54414 and human 53763 were further analyzed for the presence of a “pore domain”, also known as a “P-region domain”. A pore domain was identified in the amino acid sequence of human 54414 at about residues 229-250 of SEQ ID NO:53. A pore domain was identified in the amino acid sequence of human 53763 at about residues 426-441 of SEQ ID NO:56.

The amino acid-sequences of human 54414 and human 53763 were also analyzed for the presence of a “potassium channel signature sequence motif” (see Joiner, W. J. et al. (1998) Nat. Neurosci. 1:462-469 and references cited therein). A potassium channel signature sequence motif was identified in the amino acid sequence of human 54414 at about residues 239-246 of SEQ ID NO:53. A potassium channel signature sequence motif was identified in the amino acid sequence of human 53763 at about residues 436-441 of SEQ ID NO:56.

The amino acid sequence of human 53763 was also analyzed for the presence of a “voltage sensor motif”. A voltage sensor motif was identified in the amino acid sequence of human 53763 at about residues 348-363 of SEQ ID NO:56. Positively charged amino acid residues in the voltage sensor motif were identified about residues 348, 351, 354, 357, 360, and 363 of SEQ ID NO:50.

Isolation of the Human 67076, 67102, 44181, 67084FL, or 67084alt cDNAs

The invention is based, at least in part, on the discovery of a human gene encoding novel polypeptides, referred to herein as human 67076, 67102, 44181, 67084FL, and 67084alt. The entire sequence of the human clone 67076 was determined and found to contain an open reading frame termed human “67076.” The nucleotide sequence of the human 67076 gene is set forth in FIGS. 63A-63H and in the Sequence Listing as SEQ ID NO:58. The amino acid sequence of the human 67076 expression product is set forth in FIGS. 63A-63H and in the Sequence Listing as SEQ ID NO:59. The 67076 polypeptide comprises 1129 amino acids. The coding region (open reading frame) of SEQ ID NO:58 is set forth as SEQ ID NO:60. Clone 67076, comprising the coding region of human 67076, was deposited with the American Type Culture Collection (ATCC®), 10801 University Boulevard, Manassas, Va. 20110-2209, on ______, and assigned Accession No. ______.

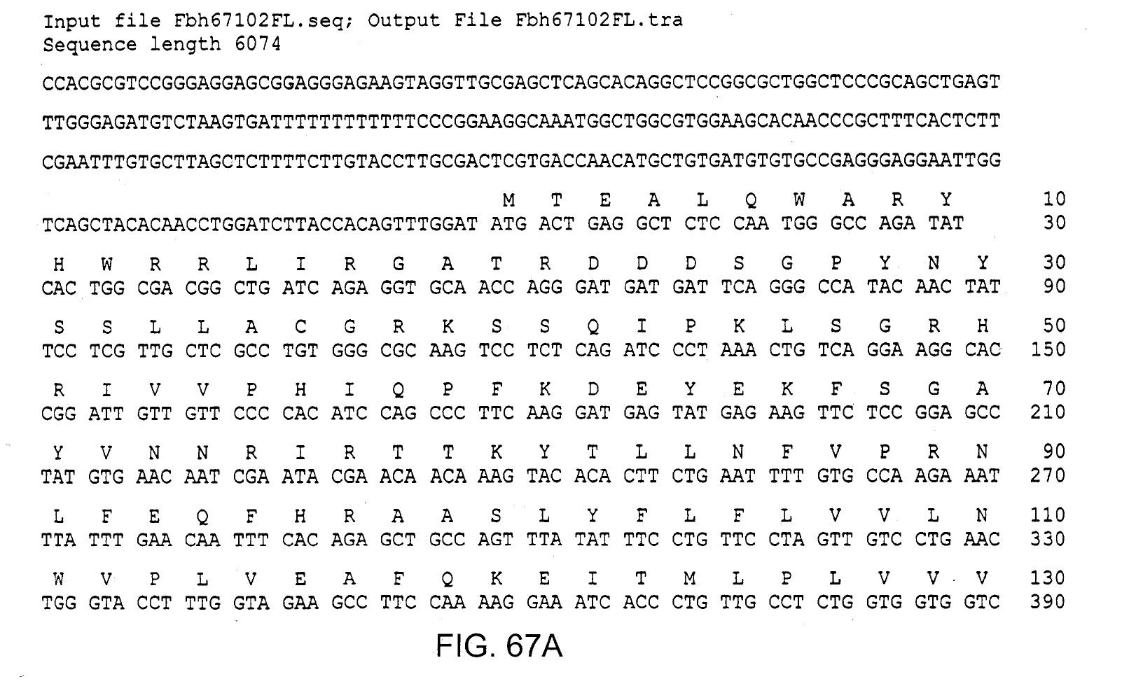

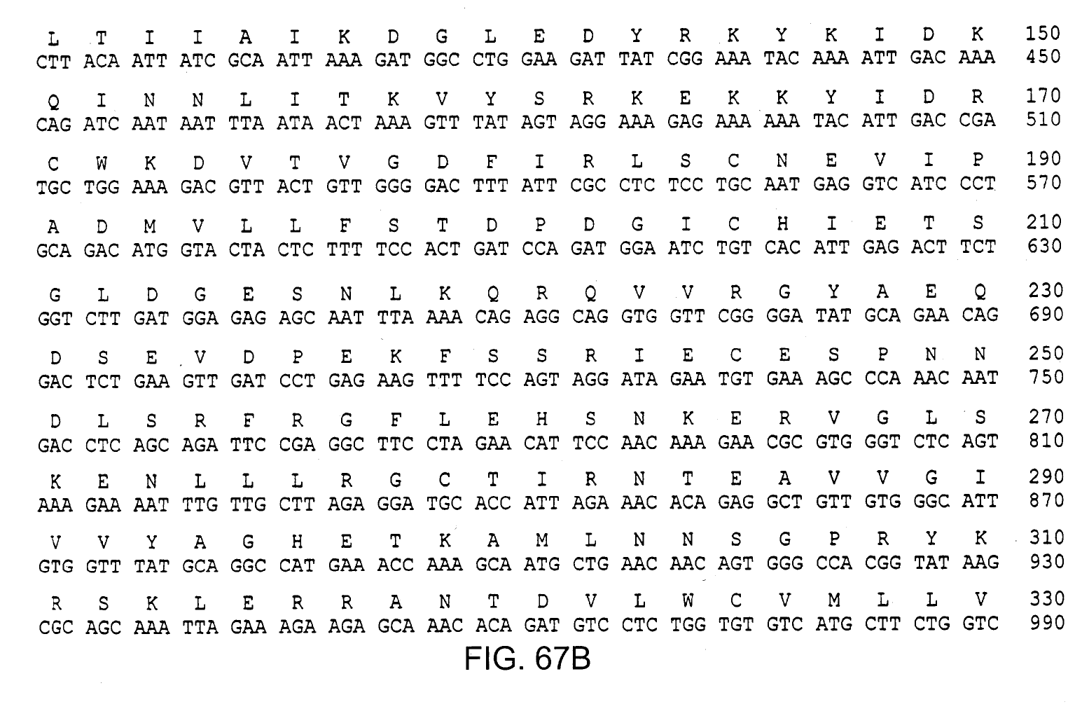

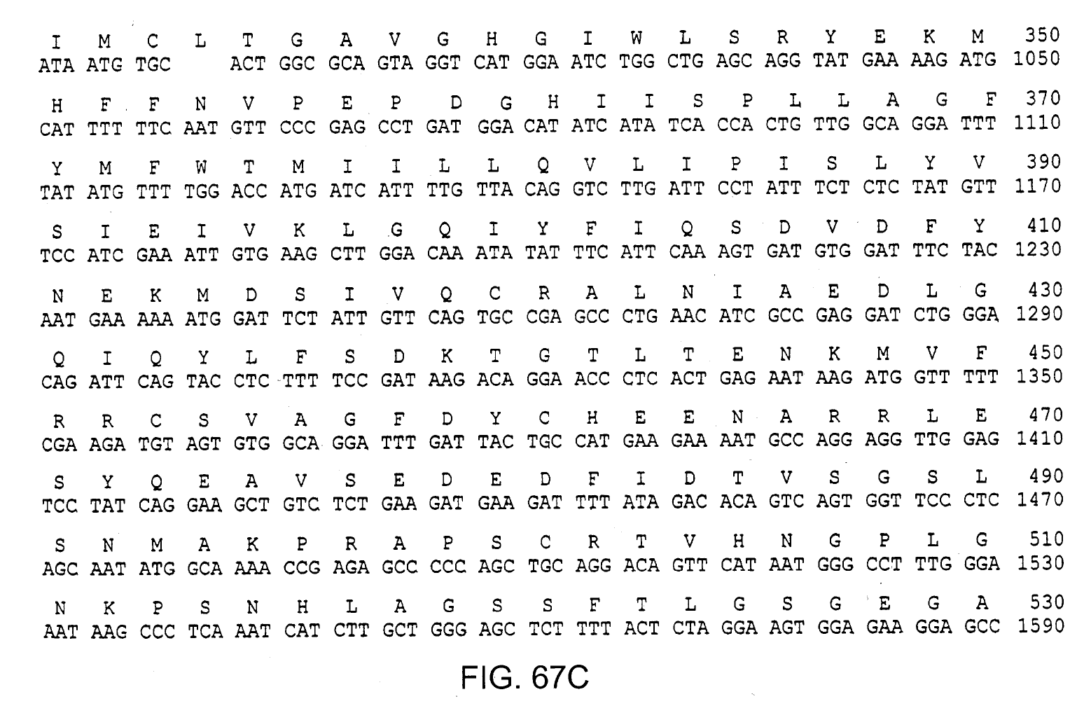

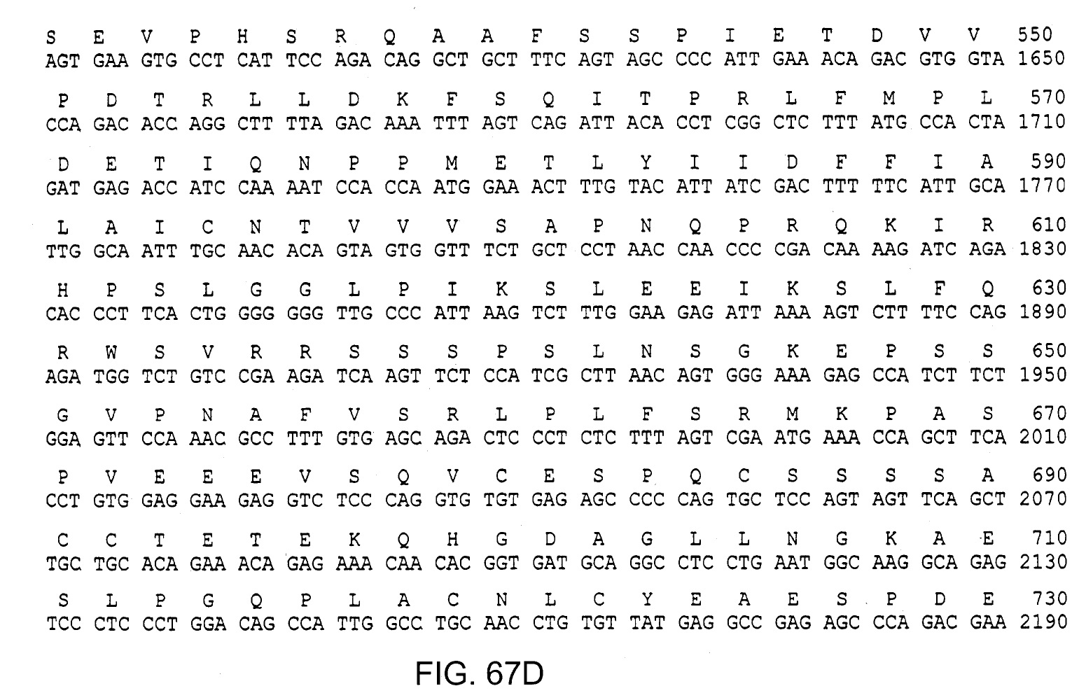

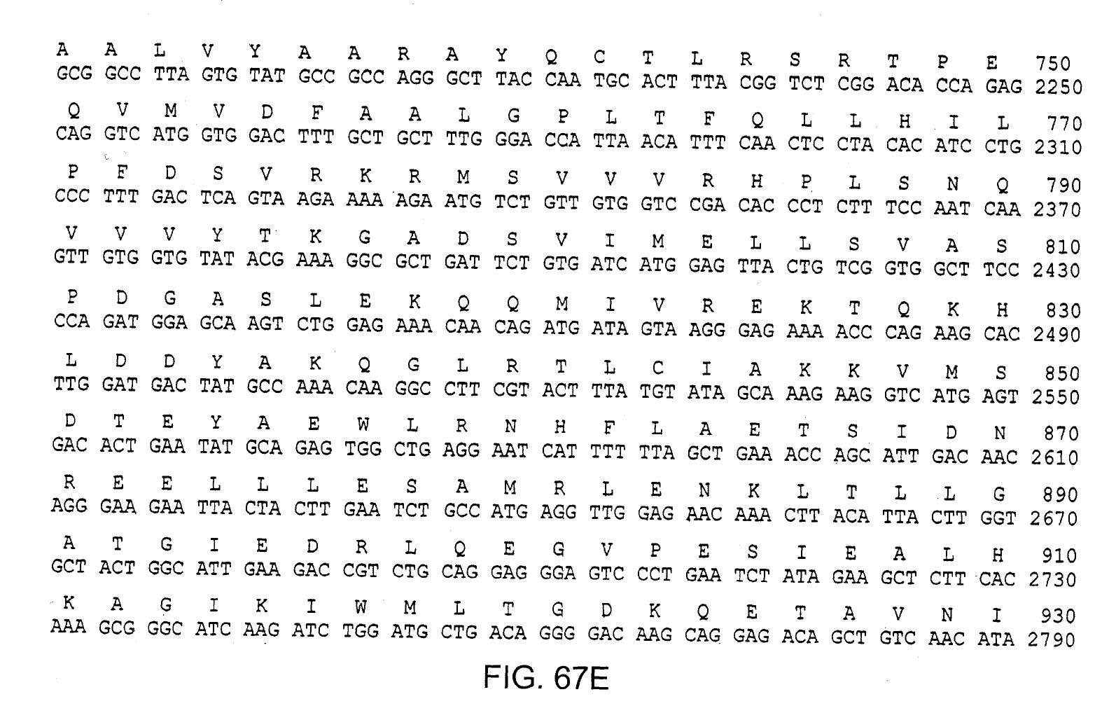

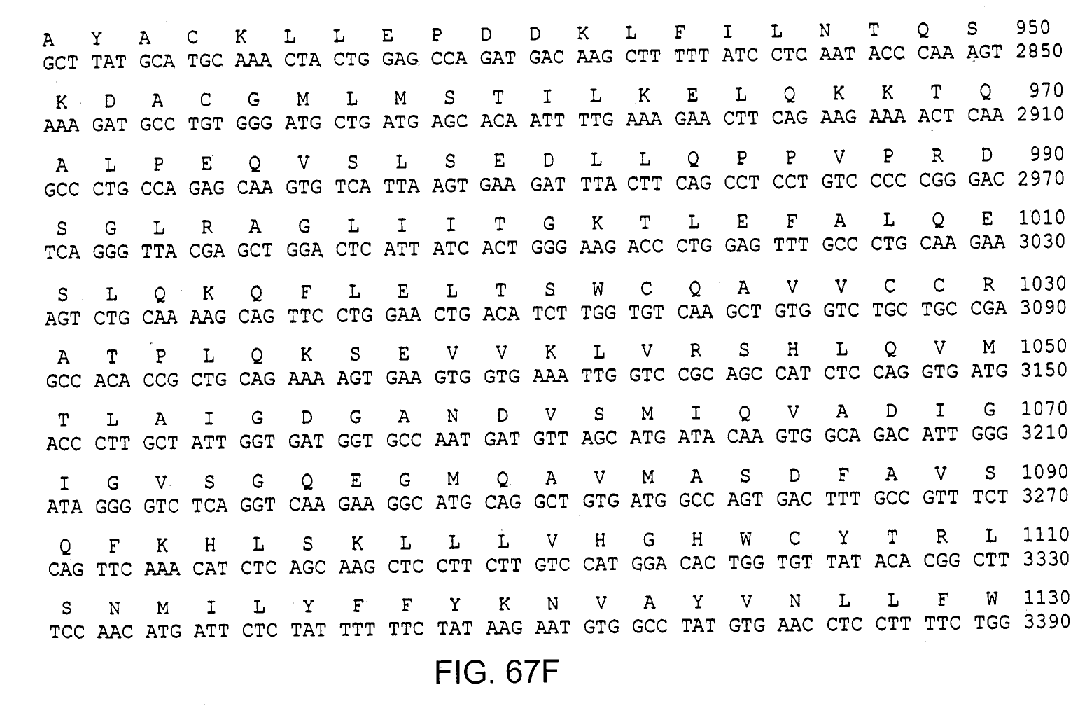

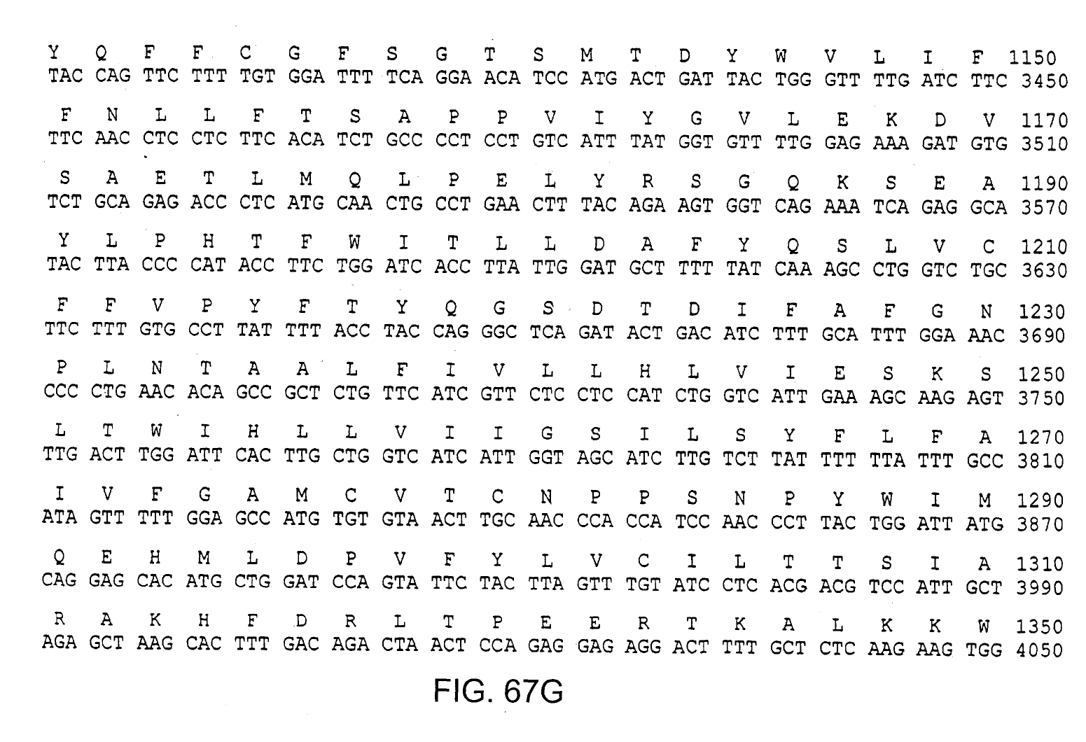

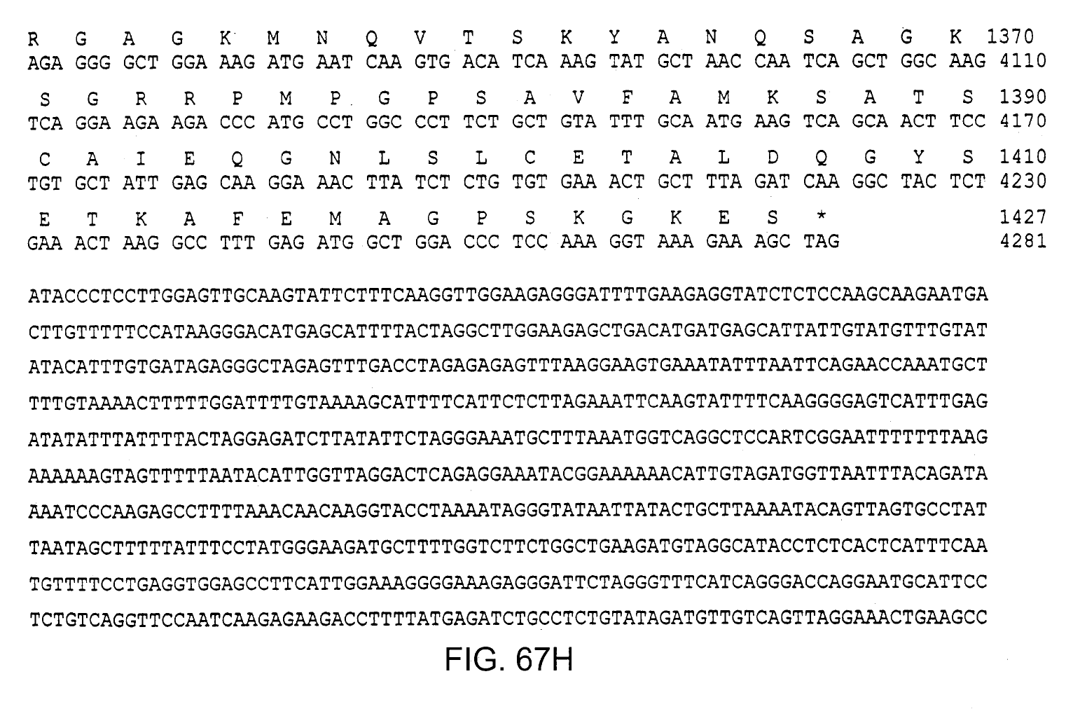

The entire sequence of the human clone 67102 as determined and found to contain an open reading frame termed human “67102.” The nucleotide sequence of the human gene is set forth in FIGS. 67A-67I and in the Sequence Listing as SEQ ID NO:61. The amino acid sequence of the human 67102 expression product is set forth in FIGS. 67A-67I and in the Sequence Listing as SEQ ID NO:62. The 67102 polypeptide comprises 1426 amino acids. The coding region (open reading frame) of SEQ ID NO:61 is set forth as SEQ ID NO:63. Clone 67102, comprising the coding region of human 67102, was deposited with the American Type Culture Collection (ATCC®), 10801 University Boulevard, Manassas, Va. 20110-2209, on ______, and assigned Accession No. ______.

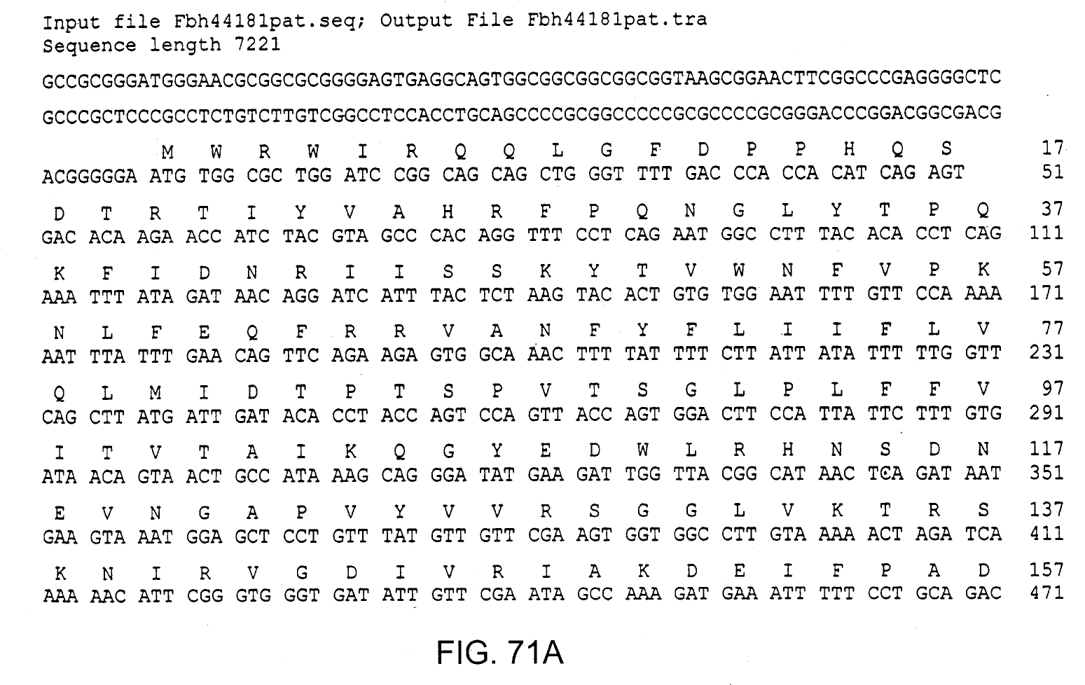

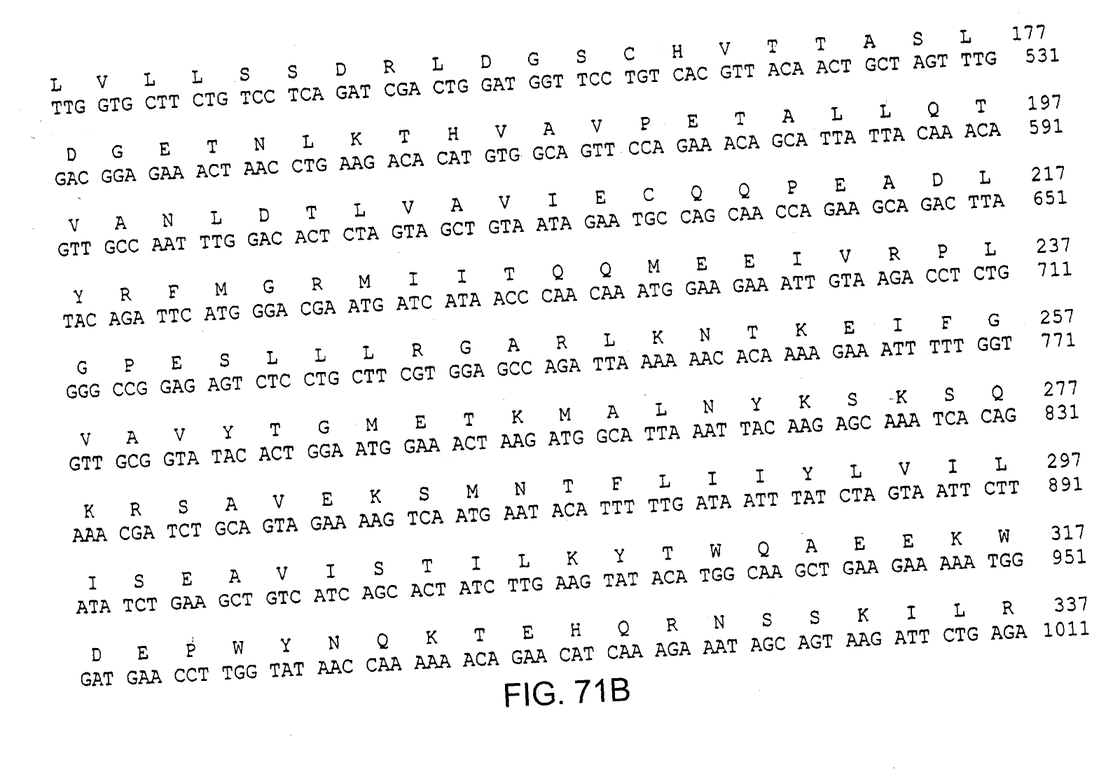

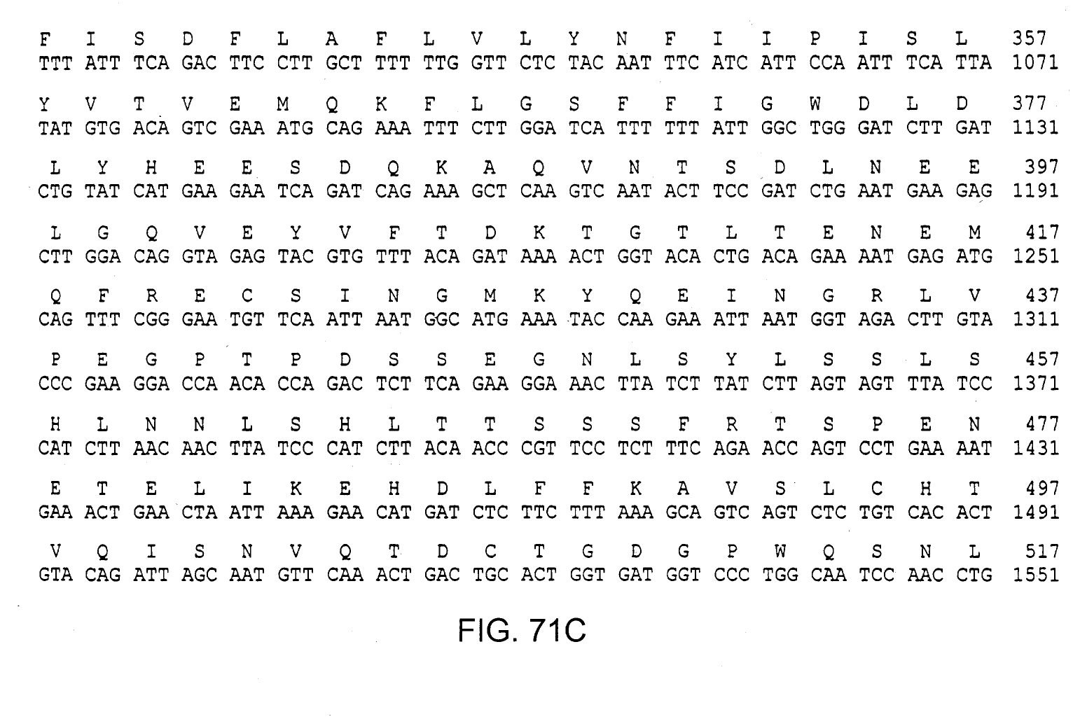

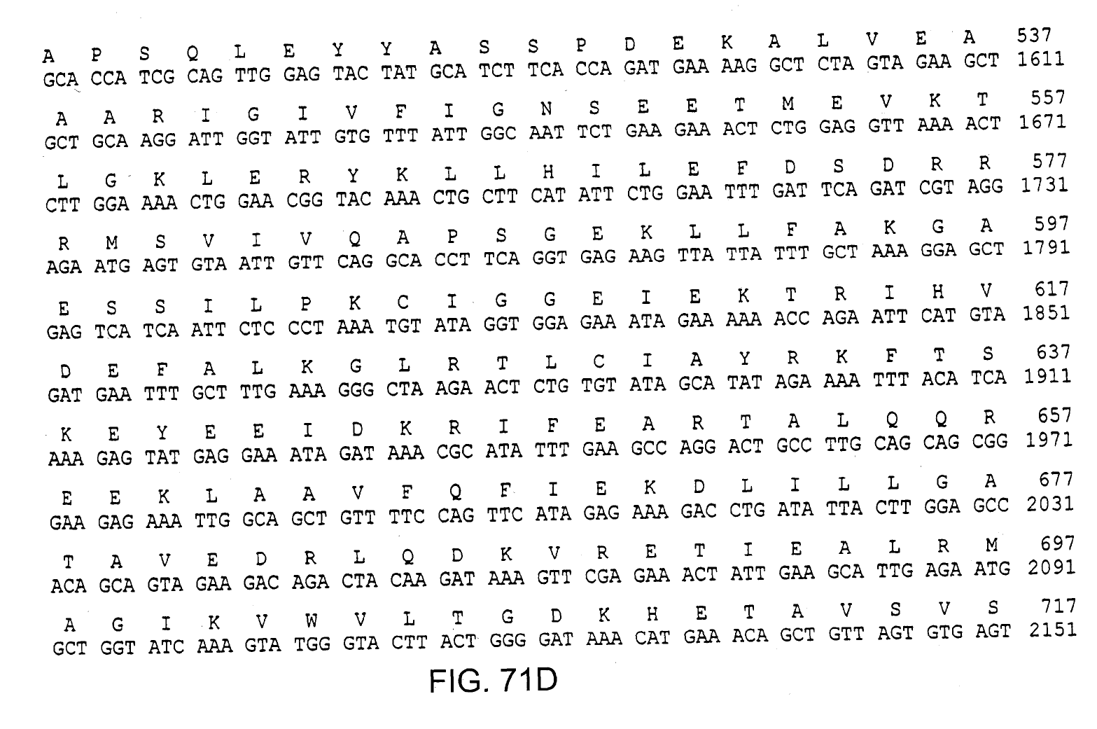

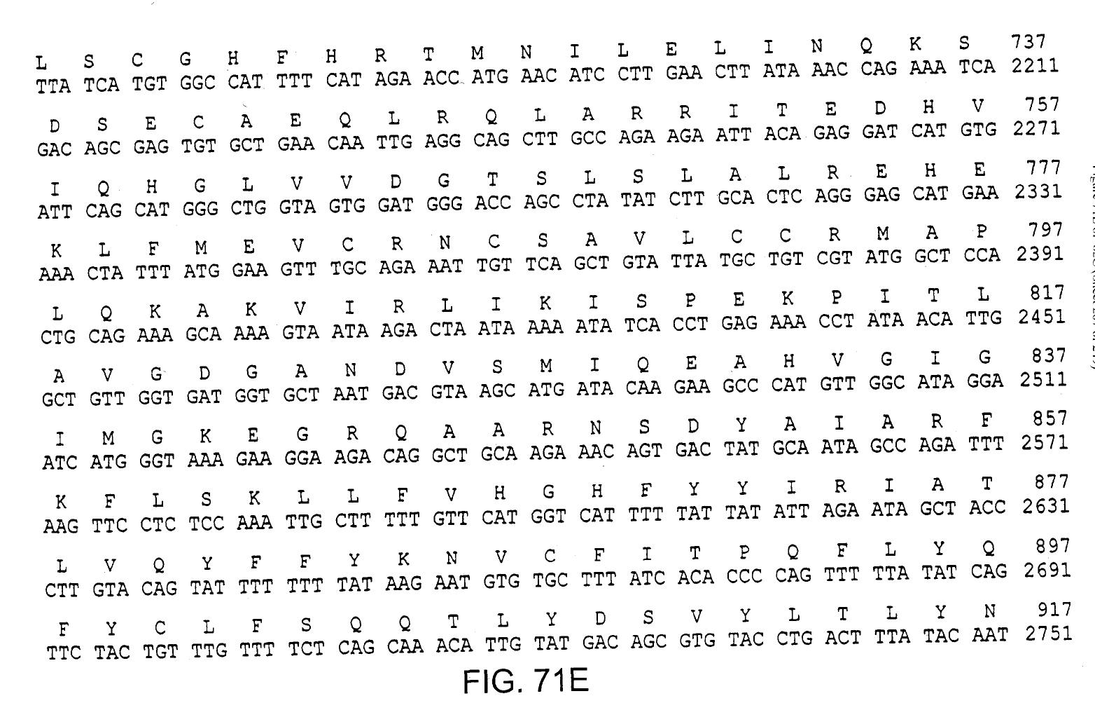

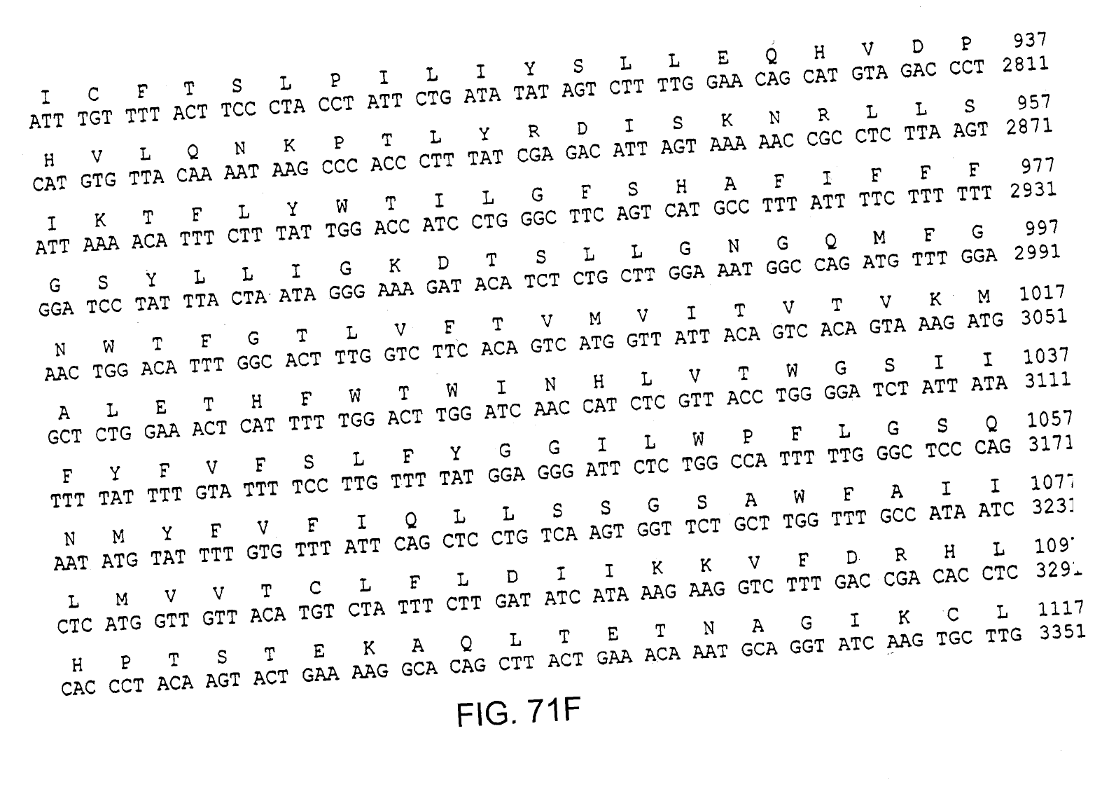

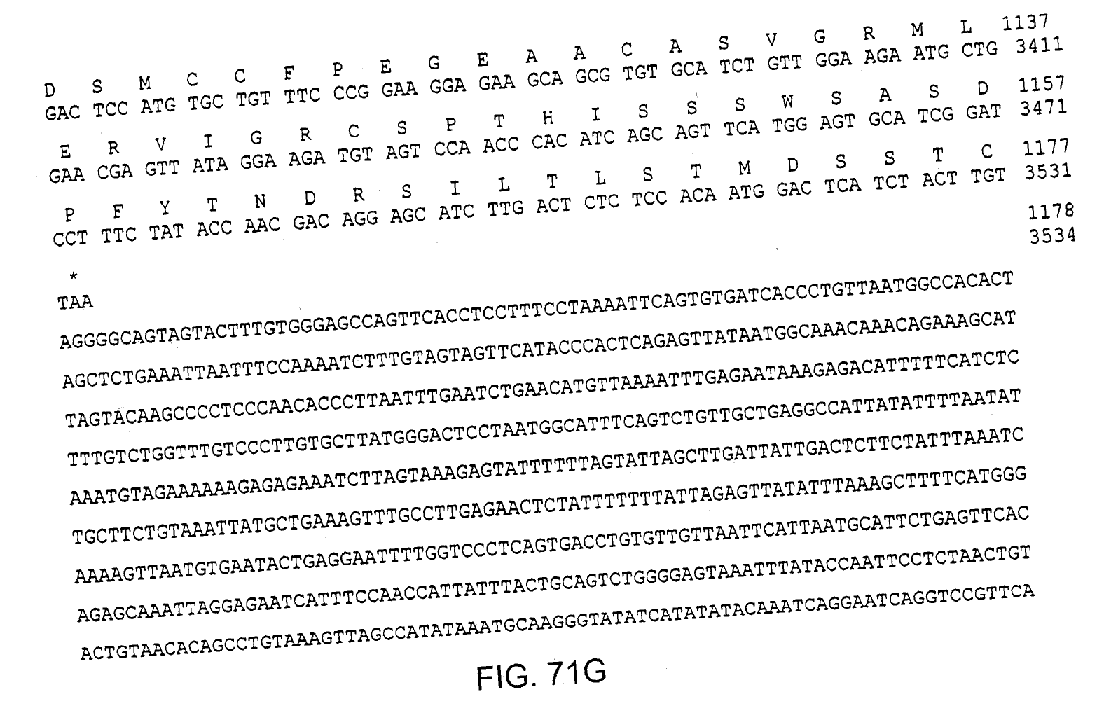

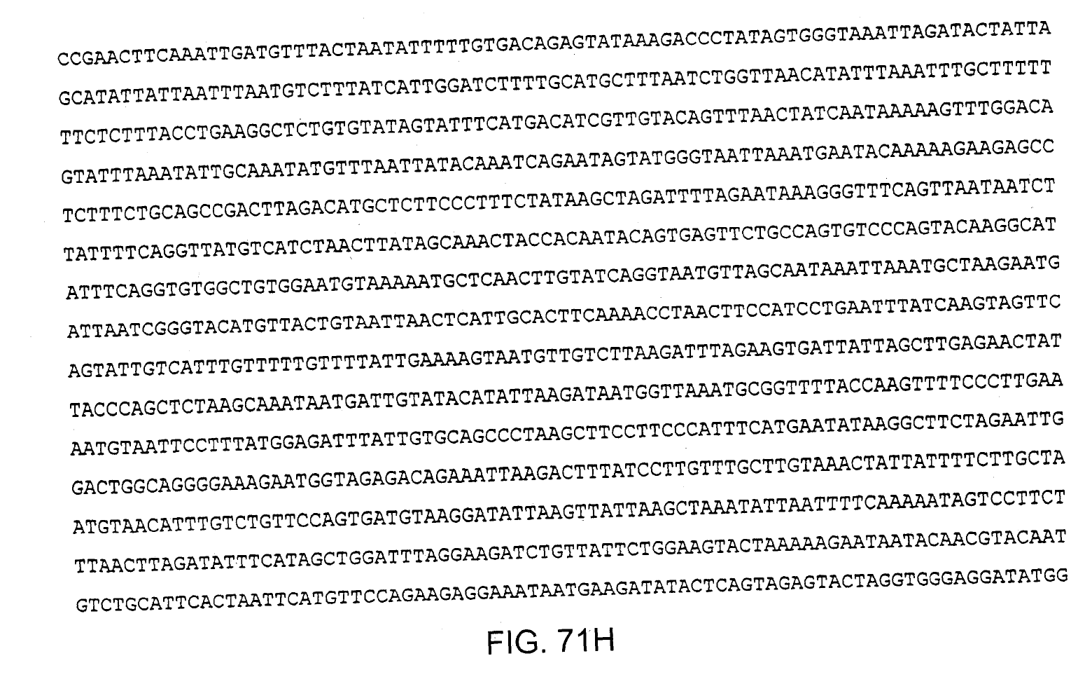

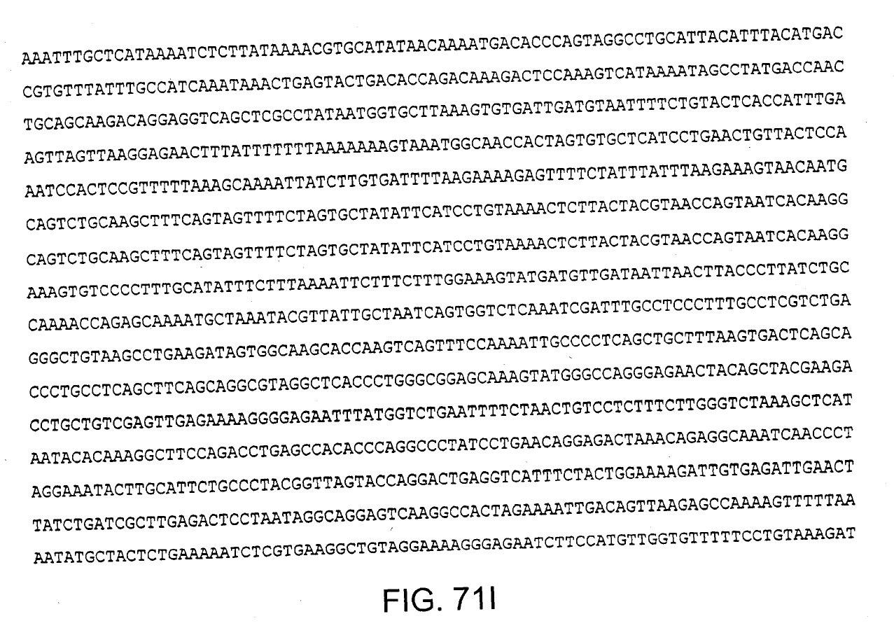

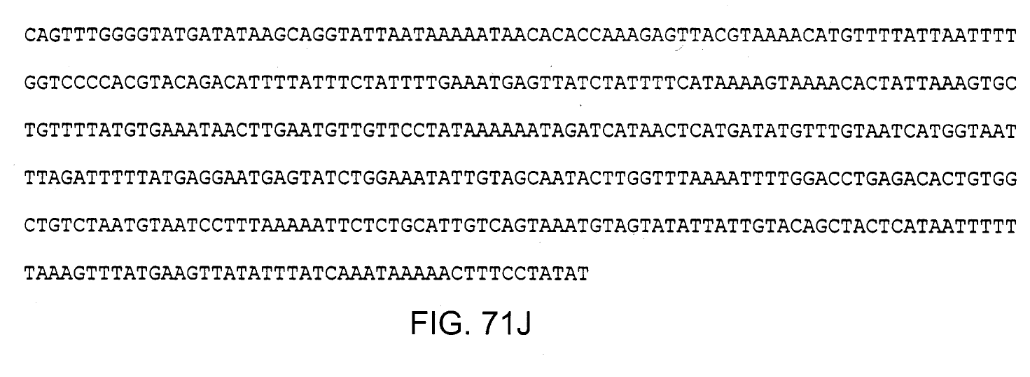

The entire sequence of the human clone 44181 was determined and found to contain an open reading frame termed human “44181.” The nucleotide sequence of the human 44181 gene is set forth in FIGS. 71A-71J and in the Sequence Listing as SEQ ID NO:64. The amino acid sequence of the human 44181 expression product is set forth in FIGS. 71A-71H and in the Sequence Listing as SEQ ID NO:65. The 44181 polypeptide comprises 1177 amino acids. The coding region (open reading frame) of SEQ ID NO:64 is set forth as SEQ ID NO:66. Clone 44181, comprising the coding region of human 44181, was deposited with the American Type Culture Collection (ATCC®), 10801 University Boulevard, Manassas, Va. 20110-2209, on ______, and assigned Accession No. ______.

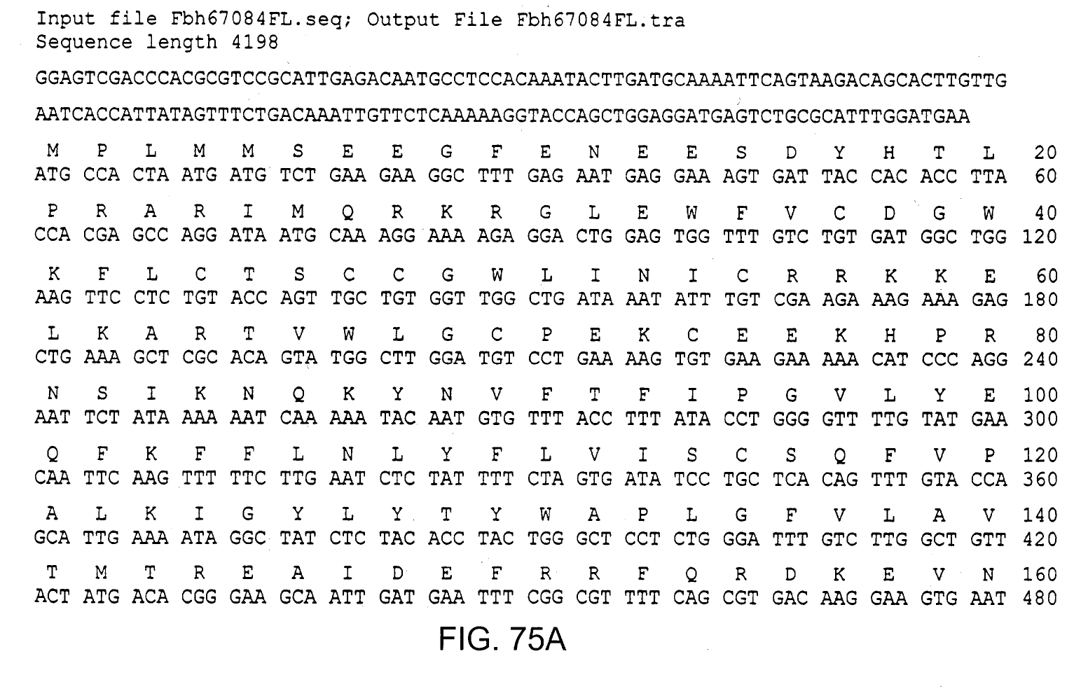

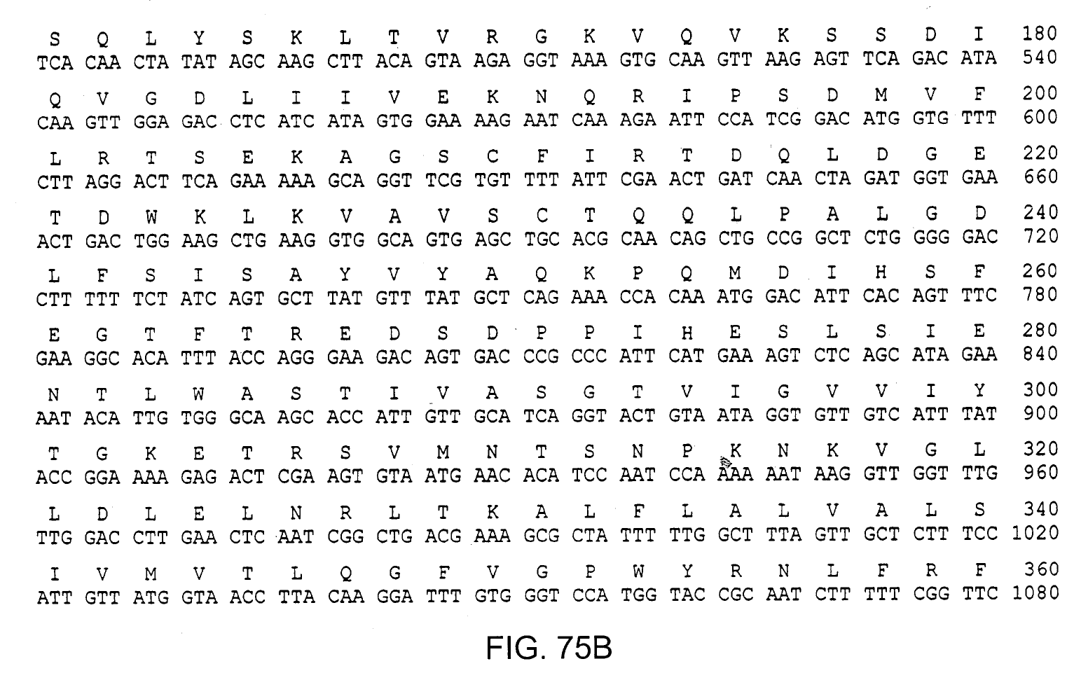

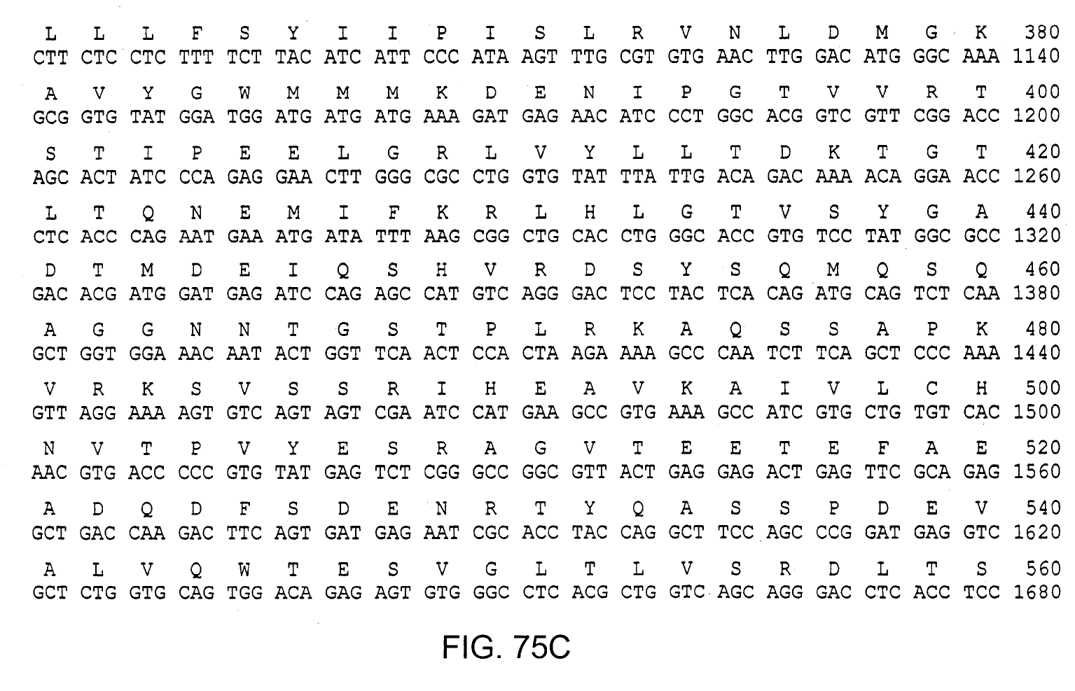

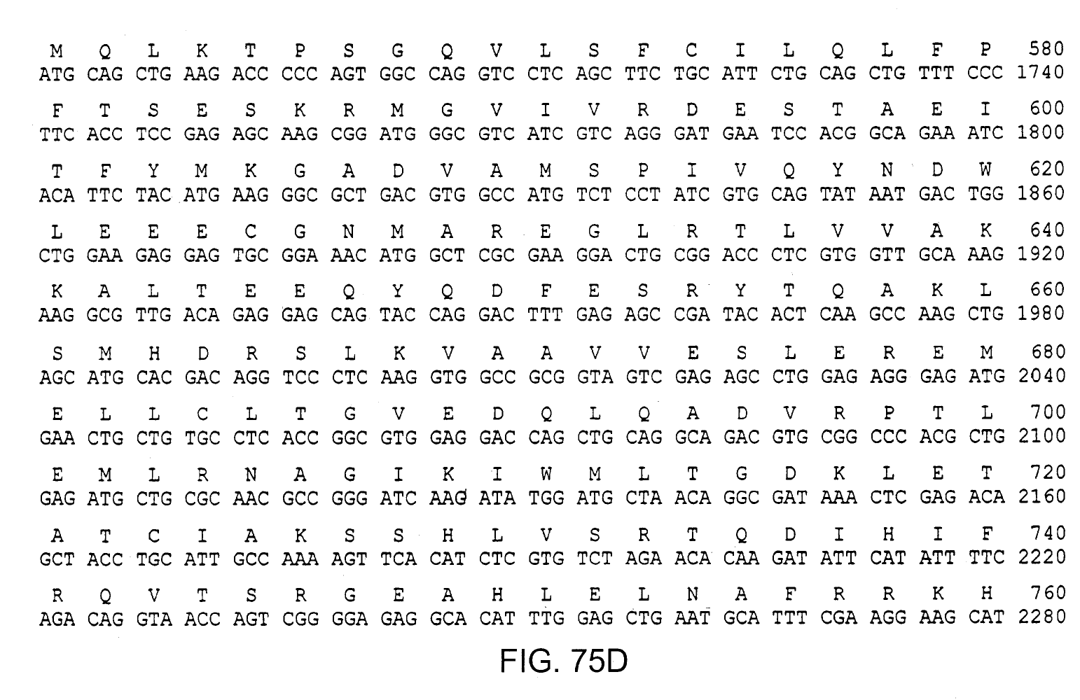

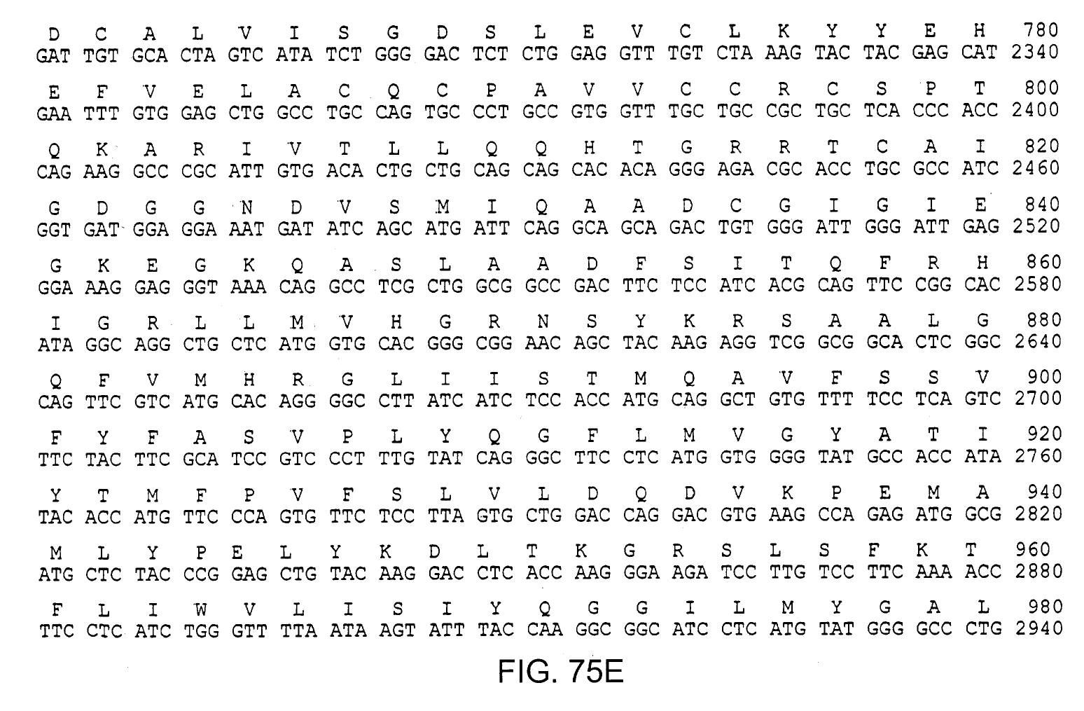

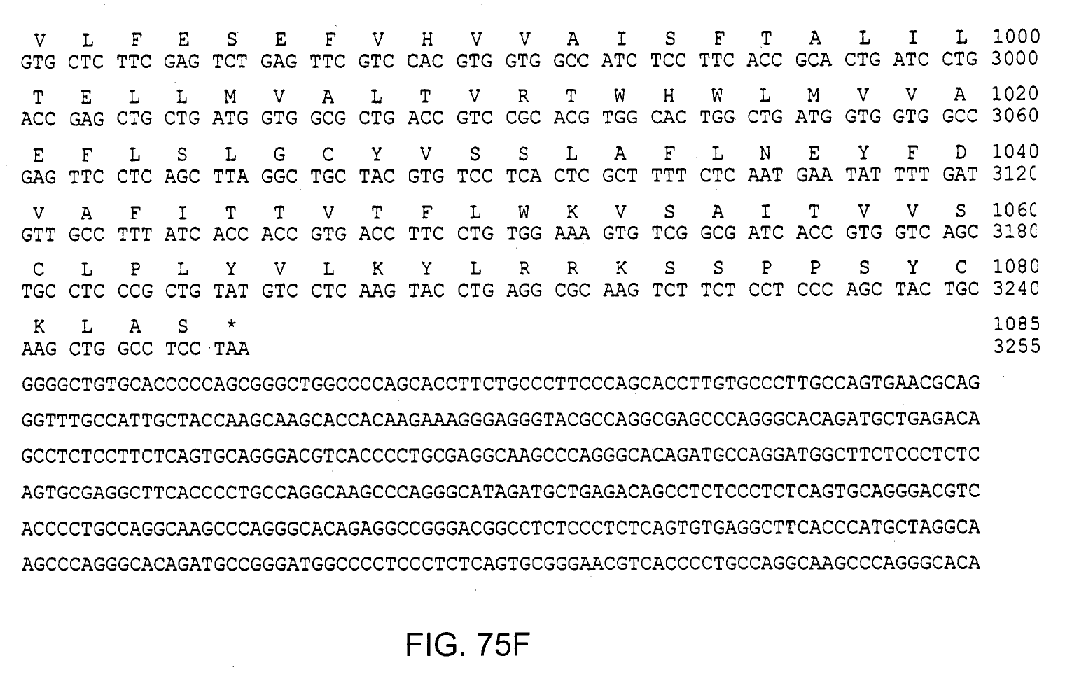

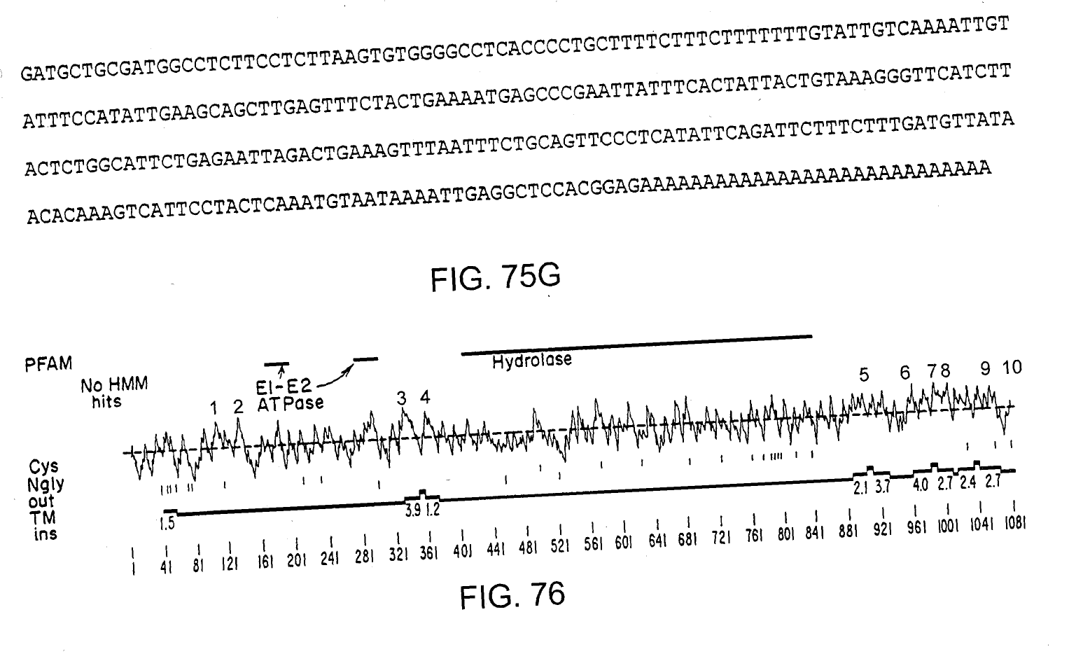

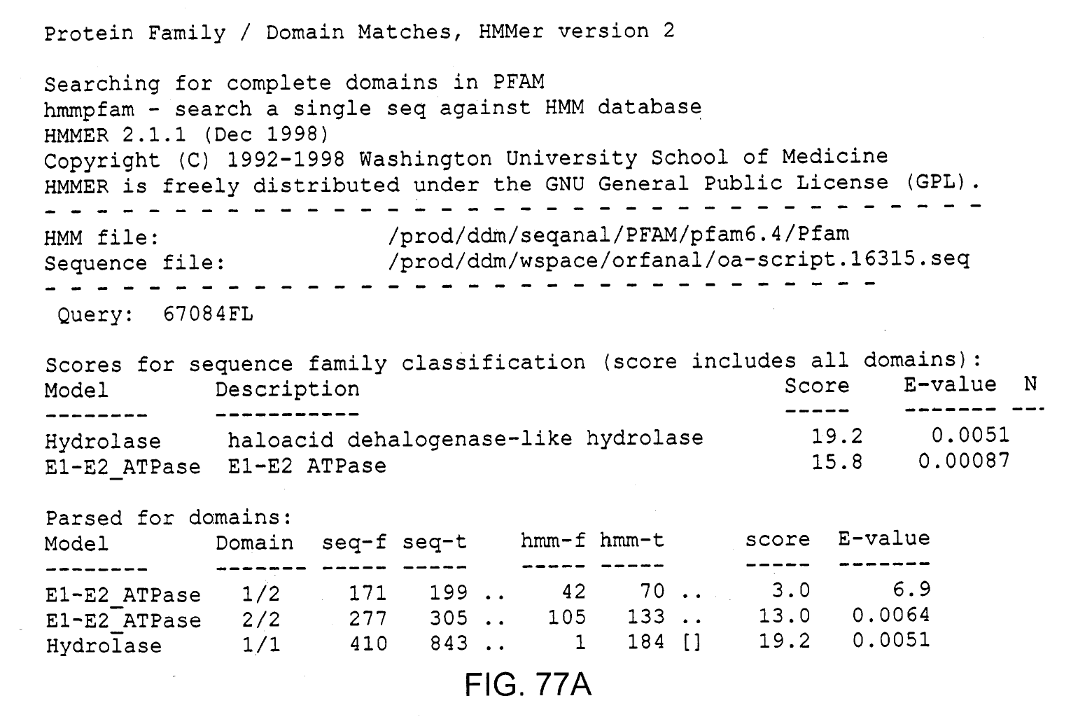

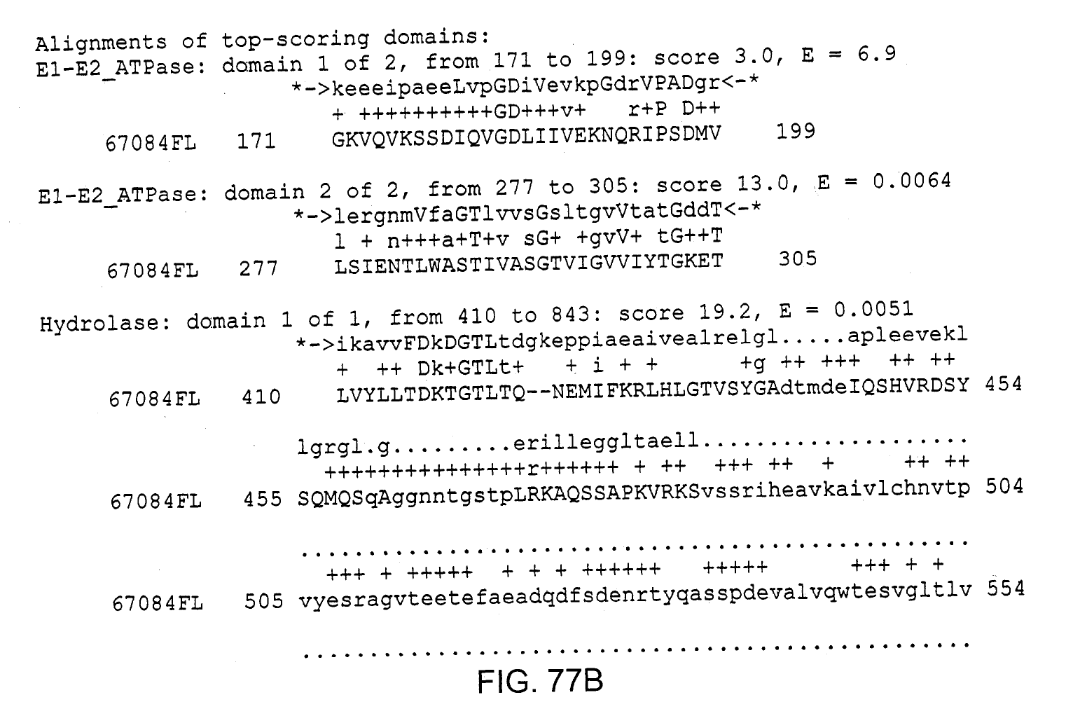

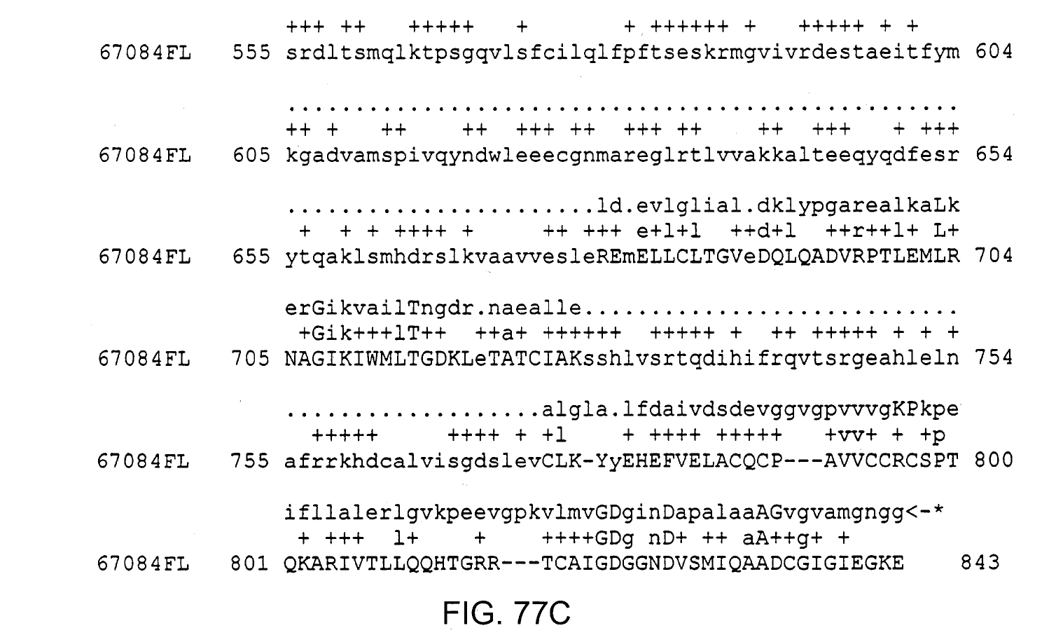

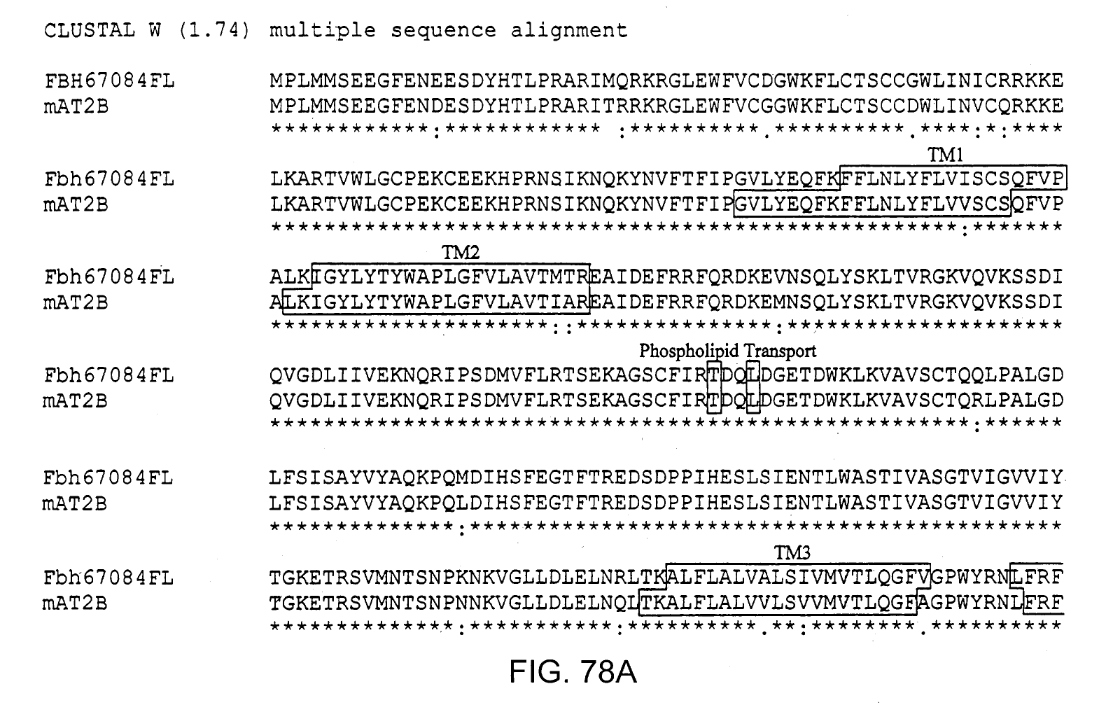

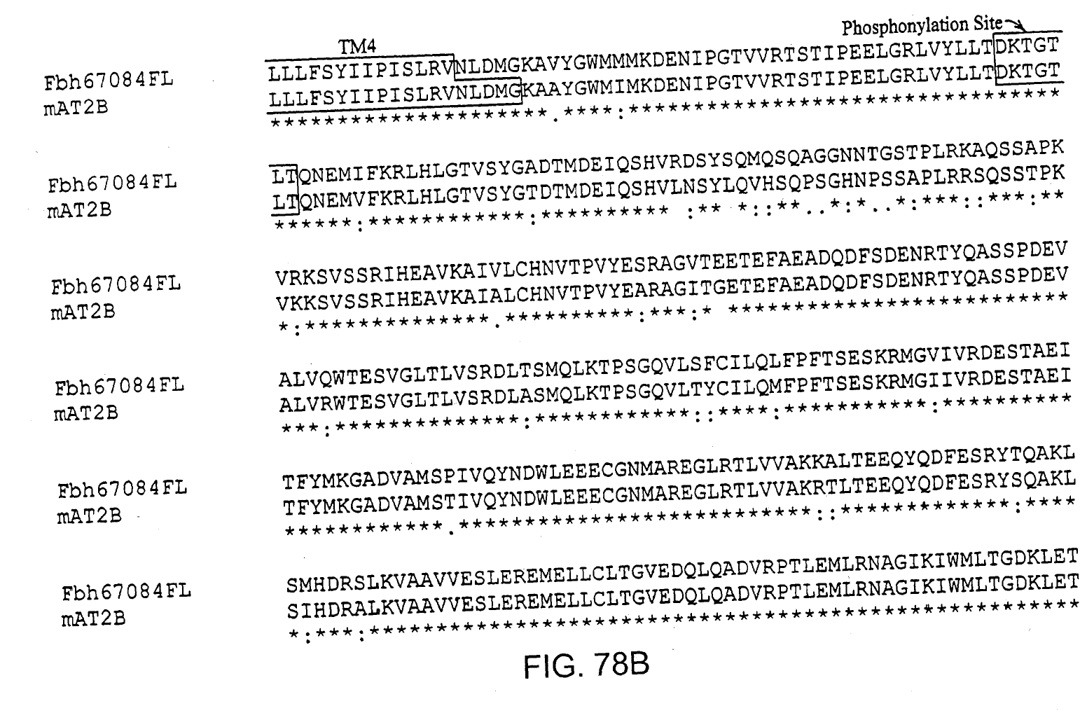

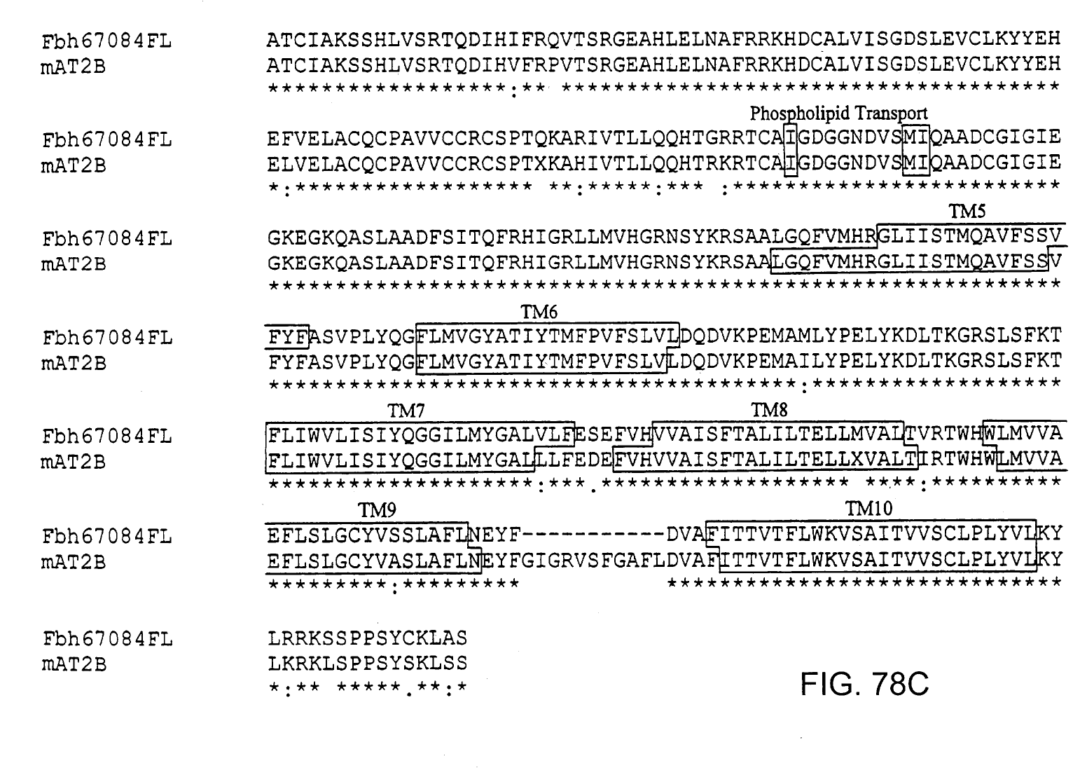

The entire sequence of the human clone 67084FL was determined and found to contain an open reading frame termed human “67084FL.” The nucleotide sequence of the human 67084FL gene is set forth in FIGS. 75A-75G and in the Sequence Listing as SEQ ID NO:67. The amino acid sequence of the human 67084FL expression product is set forth in FIGS. 75A-75G and in the Sequence Listing as SEQ ID NO:68. The 67084FL polypeptide comprises 1084 amino acids. The coding region (open reading frame) of SEQ ID NO:67 is set forth as SEQ ID NO:69. Clone 67084FL, comprising the coding region of human 67084FL, was deposited with the American Type Culture Collection (ATCC®), 10801 University Boulevard, Manassas, Va. 20110-2209, on ______, and assigned Accession No. ______.

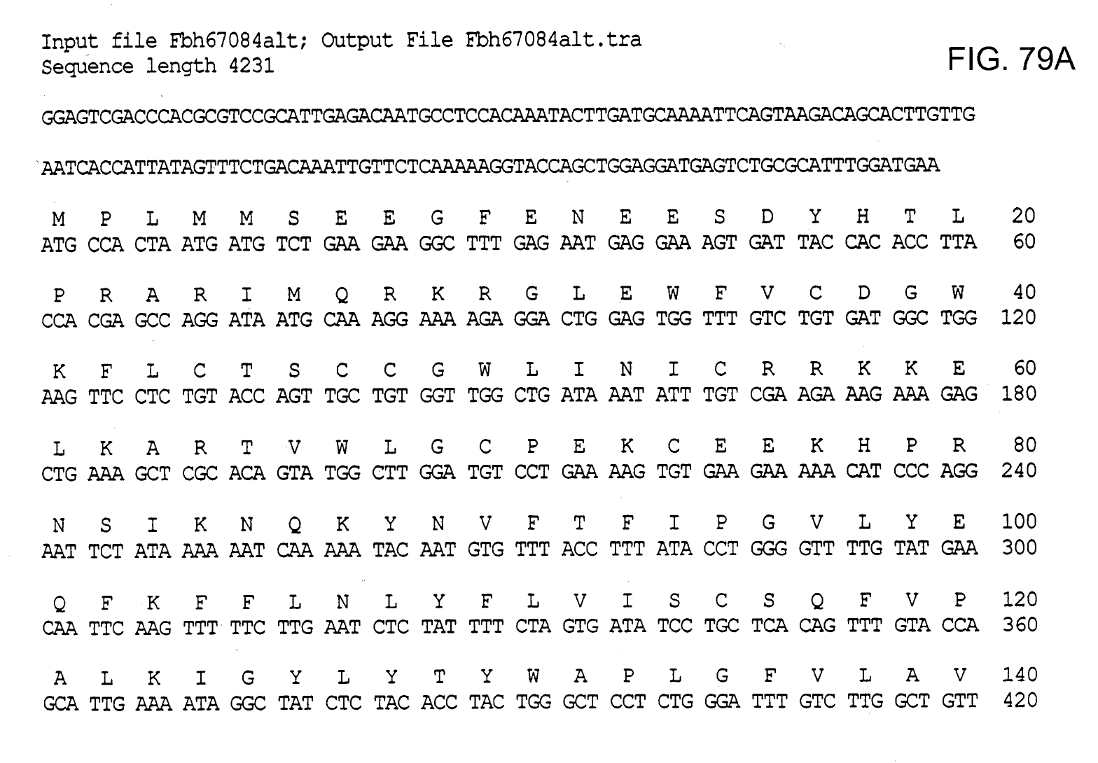

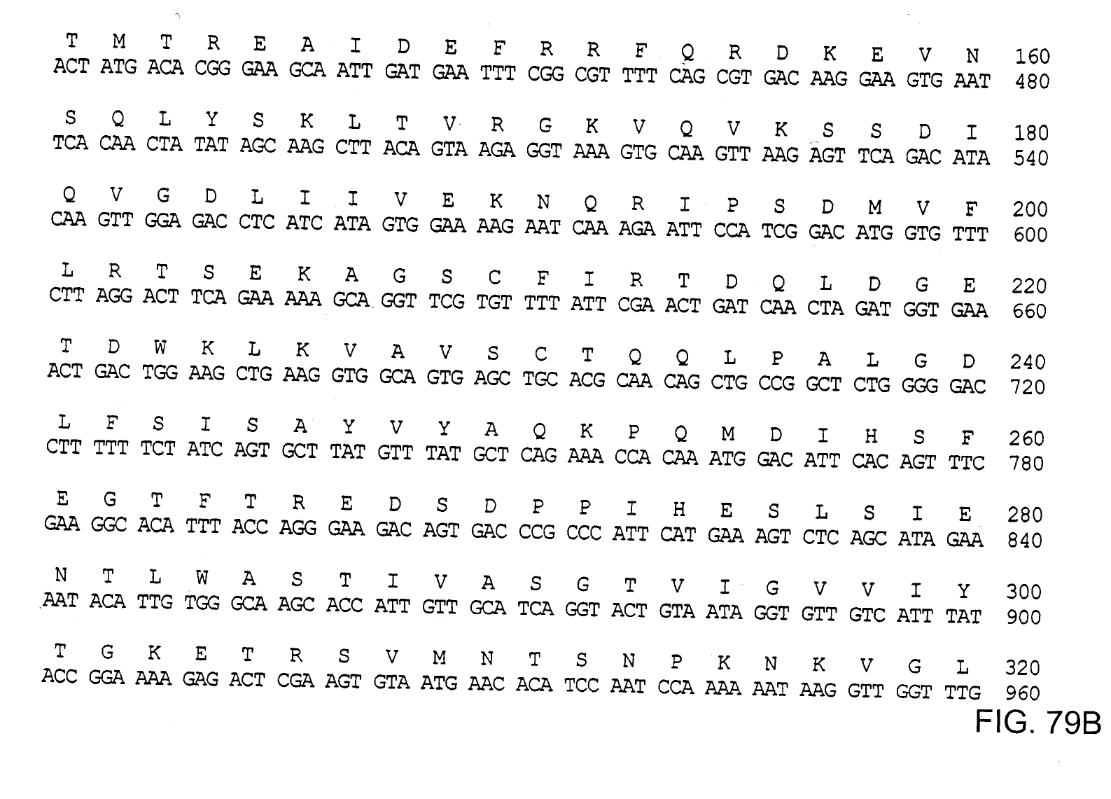

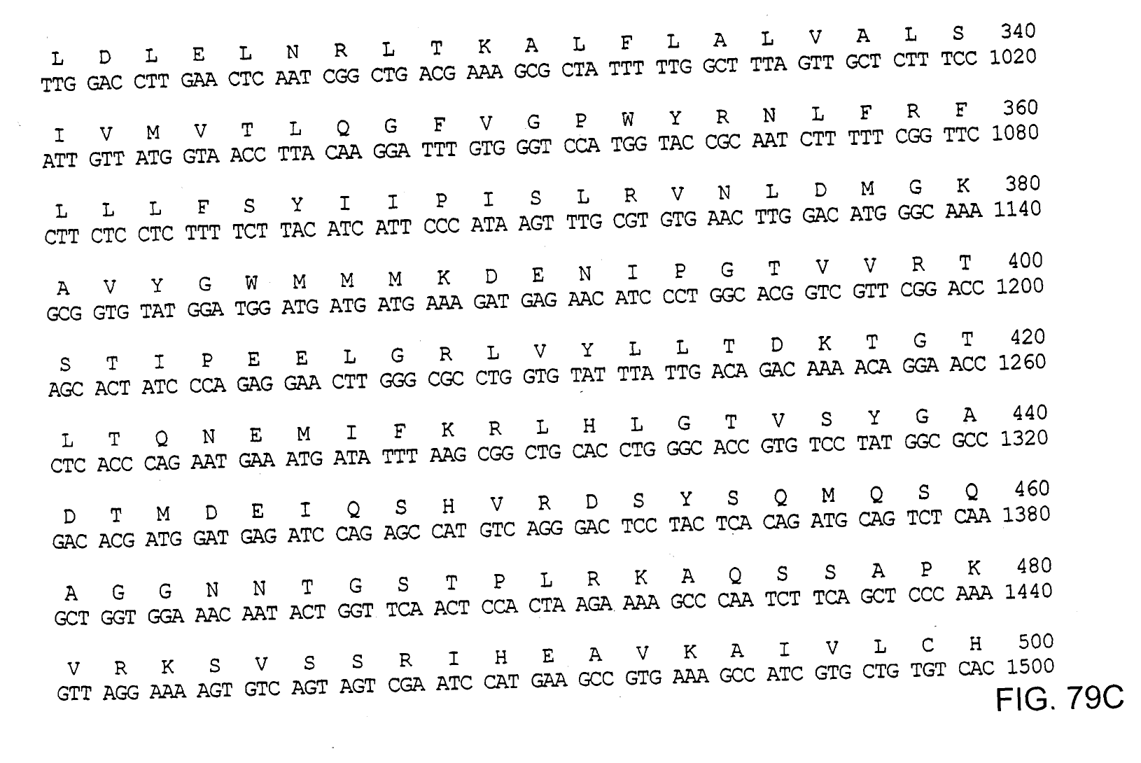

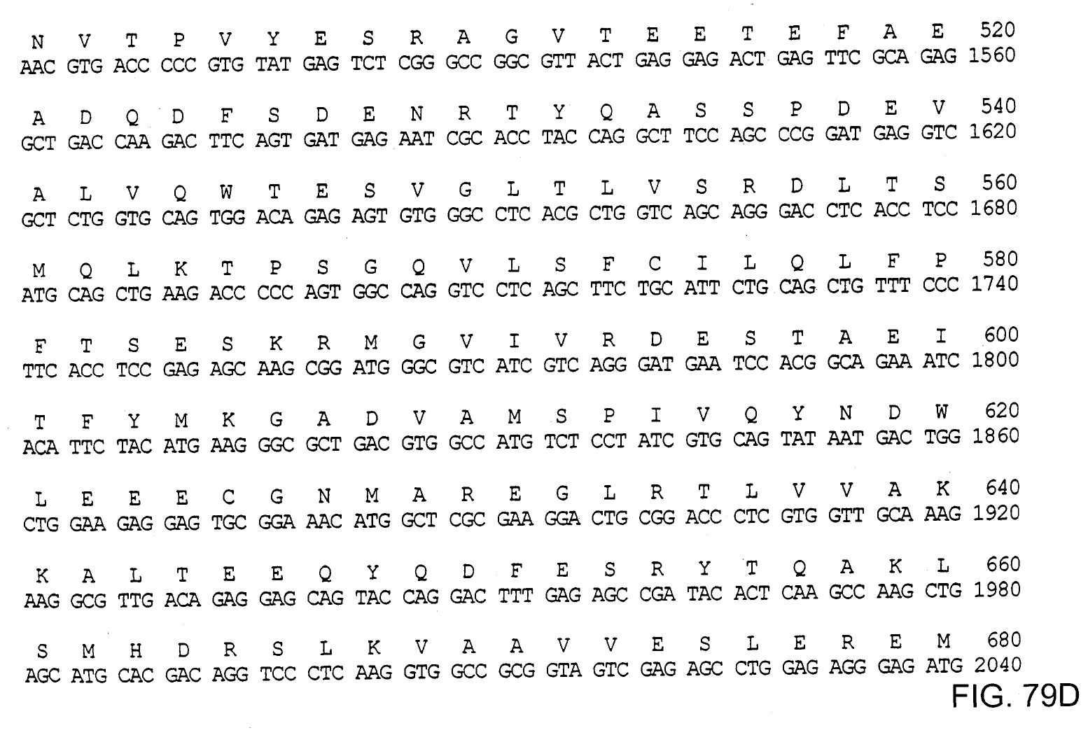

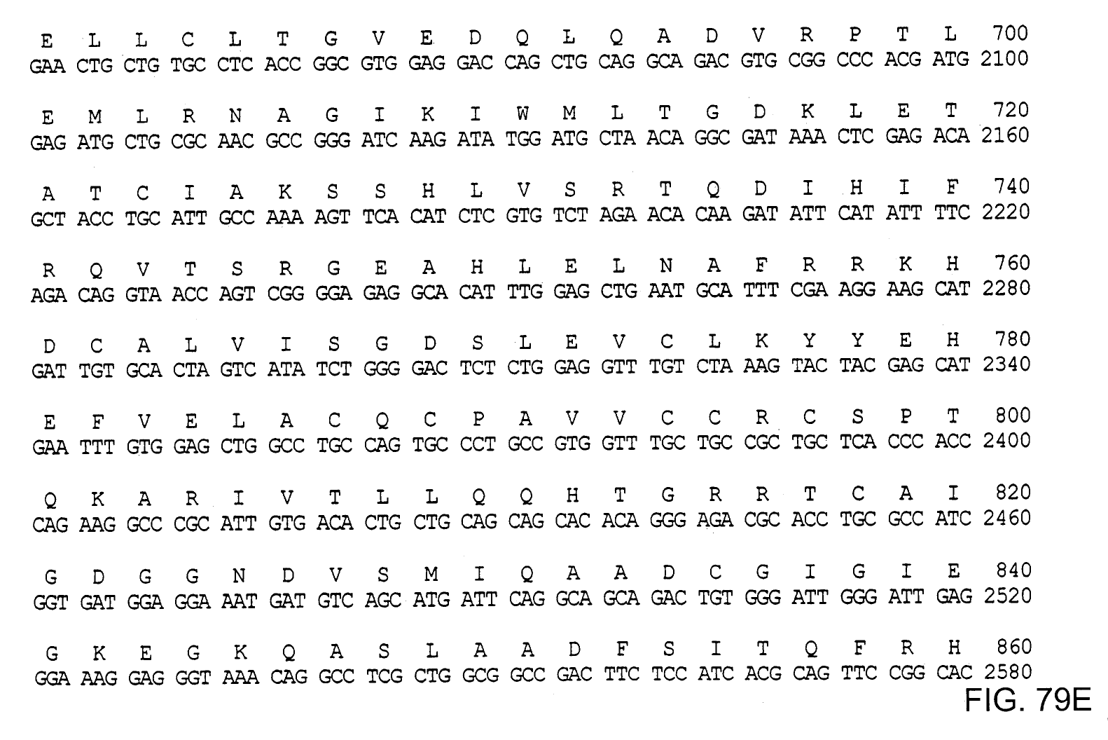

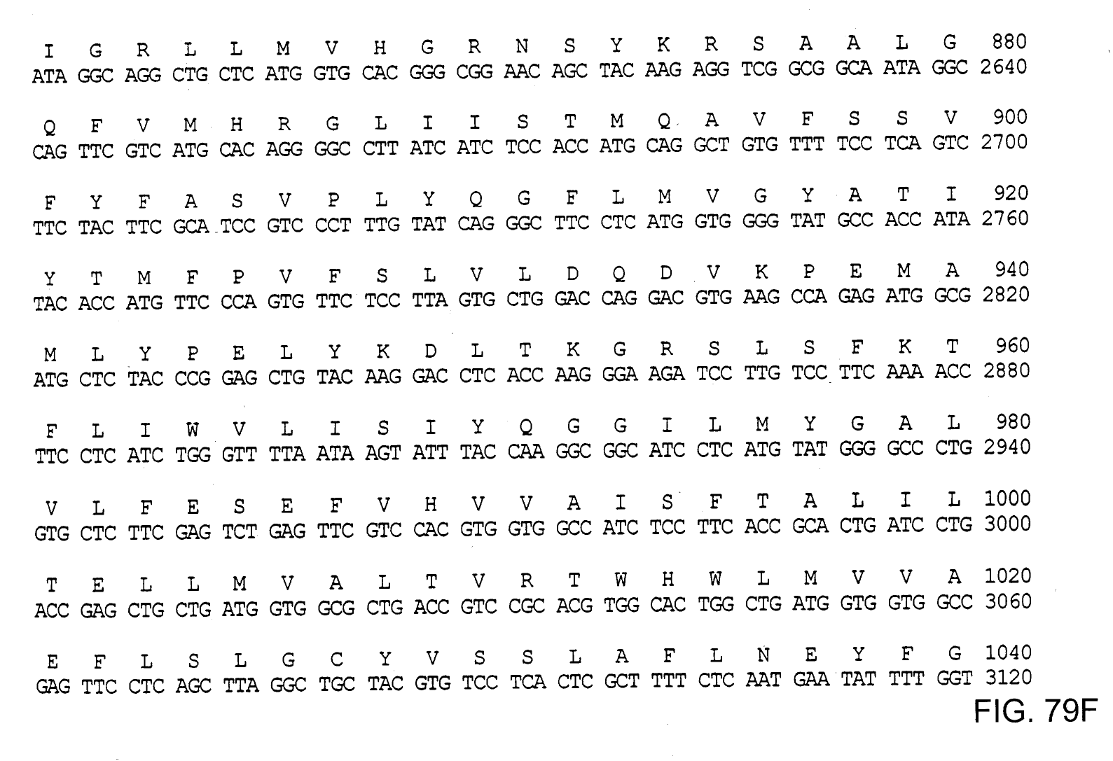

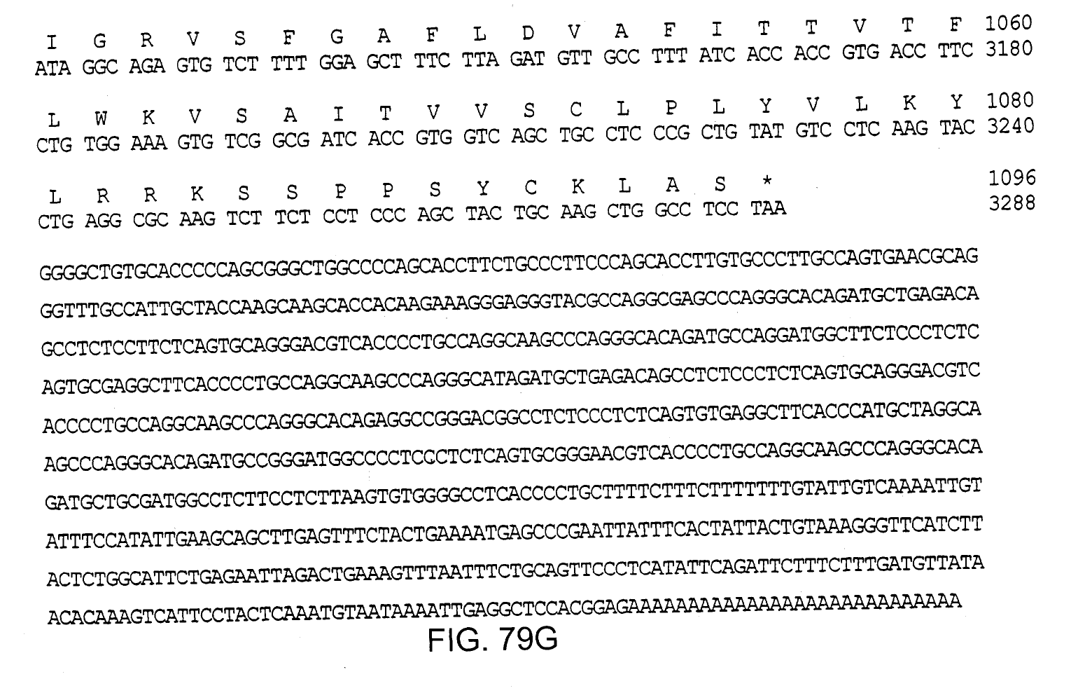

The entire sequence of the human clone 67084alt was determined and found to contain an open reading frame termed human “67084alt.” The nucleotide sequence of the human 67084alt gene is set forth in FIGS. 79A-79G and in the Sequence Listing as SEQ ID NO:70. The amino acid sequence of the human 67084alt expression product is set forth in FIGS. 79A-79G and in the Sequence Listing as SEQ ID NO:71. The 67084alt polypeptide comprises 1095 amino acids. The coding region (open reading frame) of SEQ ID NO:70 is set forth as SEQ ID NO:72. Clone 67084alt, comprising the coding region of human 67084alt, was deposited with the American Type Culture Collection (ATCC®), 10801 University Boulevard, Manassas, Va. 20110-2209, on ______, and assigned Accession No. ______.

Analysis of the Human 67076, 67102, 44181, 67084FL, or 67084alt Molecules

The amino acid sequences of human 67076, 67102, 44181, 67084FL, or 67084alt were analyzed for the presence of sequence motifs specific for P-type ATPases (as defined in Tang, X. et al. (1996) Science 272:1495-1497 and Fagan, M. J. and Saier, M. H. (1994) J. Mol. Evol. 38:57).

These analyses resulted in the identification of a P-type ATPase sequence I motif in the amino acid sequence of human 67076 at residues 173-181 of SEQ ID NO:59, in the amino acid sequence of human 67102 at residues 208-216 of SEQ ID NO:62, in the amino acid sequence of human 44181 at residues 173-181 of SEQ ID NO:65, in the amino acid sequence of human 67084FL at residues 213-221 of SEQ ID NO:68, and in the amino acid sequence of human 67084alt at residues 213-221 of SEQ ID NO:71.

These analyses also resulted in the identification of a P-type ATPase sequence 2 motif in the amino acid sequence of human 67076 at residues 406-415 of SEQ ID NO:59, in the amino acid sequence of human 67102 at residues 435-444 of SEQ ID NO:62, in the amino acid sequence of human 44181 at residues 404-413 of SEQ ID NO:65, in the amino acid sequence of human 67084FL at residues 413-422 of SEQ ID NO:68, and in the amino acid sequence of human 67084alt at residues 413-422 of SEQ ID NO:71.

These analyses further resulted in the identification of a P-type ATPase sequence 3 motif in the amino acid sequence of human 67076 at residues 813-824 of SEQ ID NO:59, in the amino acid sequence of human 67102 at residues 1054-1064 of SEQ ID NO:62, in the amino acid sequence of human 44181 at residues 819-829 of SEQ ID NO:65, in the amino acid sequence of human 67084FL at residues 820-830 of SEQ ID NO:68, and in the amino acid sequence of human 67084alt at residues 820-830 of SEQ ID NO:71.

The amino acid sequences of human 67076, 67102, 44181, 67084FL, and 67084alt were also analyzed for the presence of phospholipid transporter specific amino acid residues (as defined in Tang, X. et al. (1996) Science 272:1495-1497). These analyses also resulted in the identification of phospholipid transporter specific amino acid residues in the amino acid sequence of human 67076 at residues 174, 177, 407, 813, 823, and 824 of SEQ ID NO:59. These analyses resulted in the identification of phospholipid transporter specific amino acid residues 208, 209, 212, 436, 1054, 1063, and 1064 in the amino acid sequence of human 67102 at residues of SEQ ID NO:62. These analyses further resulted in the identification of phospholipid transporter specific amino acid residues 174, 177, 405, 819, 928, and 929 in the amino acid sequence of human 44181 at residues of SEQ ID NO:65. These analyses further resulted in the identification of phospholipid transporter specific amino acid residues 214, 217, 820, 829, and 830 in the amino acid sequence of human 67084FL at residues of SEQ ID NO:68. These analyses still further resulted in the identification of phospholipid transporter specific amino acid residues 214, 217, 820, 829, and 830 in the amino acid sequence of human 67084alt at residues of SEQ ID NO:71.

The amino acid sequences of human 67076, 67102, 44181, 67084FL, and 67084alt were also analyzed for the presence of extramembrane domains. An N-terminal large extramembrane domain was identified in the amino acid sequence of human 67076 at residues 105-291 of SEQ ID NO:59. A C-terminal large extramembrane domain was identified in the amino acid sequence of human 67076 at residues 366-872 of SEQ ID NO:59. An N-terminal large extramembrane domain was identified in the amino acid sequence of human 67102 at residues 141-321 of SEQ ID NO:62. A C-terminal large extramembrane domain was identified in the amino acid sequence of human 67102 at residues 391-581 of SEQ ID NO:62. An N-terminal large extramembrane domain was identified in the amino acid sequence of human 44181 at residues 105-289 of SEQ ID NO:65. A C-terminal large extramembrane domain was identified in the amino acid sequence of human 44181 at residues 364-877 of SEQ ID NO:65. An N-terminal large extramembrane domain was identified in the amino acid sequence of human 67084FL at residues 145-330 of SEQ ID NO:68. A C-terminal large extramembrane domain was identified in the amino acid sequence of human 67084FL at residues 380-886 of SEQ ID NO:68. An N-terminal large extramembrane domain was identified in the amino acid sequence of human 67084alt at residues 145-330 of SEQ ID NO:71. A C-terminal large extramembrane domain was identified in the amino acid sequence of human 67084alt at residues 380-886 of SEQ ID NO:71.

The amino acid sequence of human 67076 was analyzed using the program PSORT to predict the localization of the proteins within the cell. This program assesses the presence of different targeting and localization amino acid sequences within the query sequence. The results of this analysis predict that human 67076 may be localized to the endoplasmic reticulum.

Searches of the amino acid sequence of human 67076 were further performed against the Prosite database. These searches resulted in the identification in the amino acid sequence of human 67076 of a number of potential N-glycosylation sites at amino acid residues121-124, 392-395, 761-764, 992-995, and 1098-1101 of SEQ ID NO:59, a number of potential cAMP-and cGMP-dependent protein kinase phosphorylation sites at amino acid residues135-138, 545-548, 1091-1094, and 1102-1105 of SEQ ID NO:59, a number of potential protein kinase C phosphorylation sites at amino acid residues 47-49, 138-140, 204-206, 250-252, 254-256, 278-280, 308-310, 328-330, 334-336, 408-410, 680-682, 701-703, 708-710, 733-735, 736-738, 1008-1010, 1094-1096, 1100-1102, 1109-1111, and 1113-1115 of SEQ ID NO:59, a number of casein kinase II phosphorylation sites at amino acid residues 30-33, 264-267, 282-285, 328-331, 413-416, 442-445, 449-452, 494-497, 646-649, 693-696, 704-707, 762-765, 813-816, 924-927, 982-985, and 1121-1124 of SEQ ID NO:59, a number of potential tyrosine kinase phosphorylation sites at amino acid residues 252-258, 739-747 of SEQ ID NO:59, a number of N-myristoylation sites at amino acid residues 388-393, 440-445, 482-487, 514-519, 564-569, 753-758, and 807-812 of SEQ ID NO:59, an ATP/GTP-binding site motif (P-loop) at amino acid residues 271-278 of SEQ ID NO:59, and an E1-E2 ATPases phosphorylation site at amino acid residues 409-415 of SEQ ID NO:59.

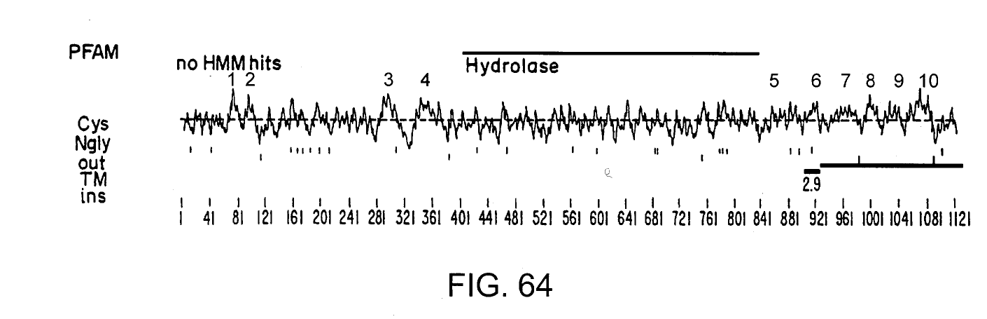

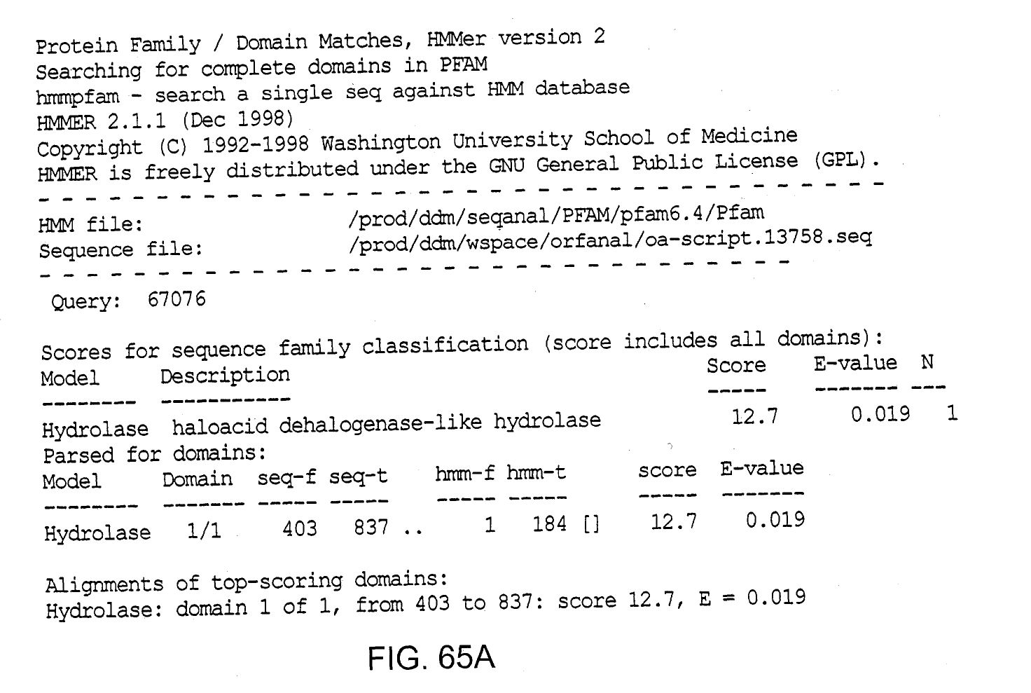

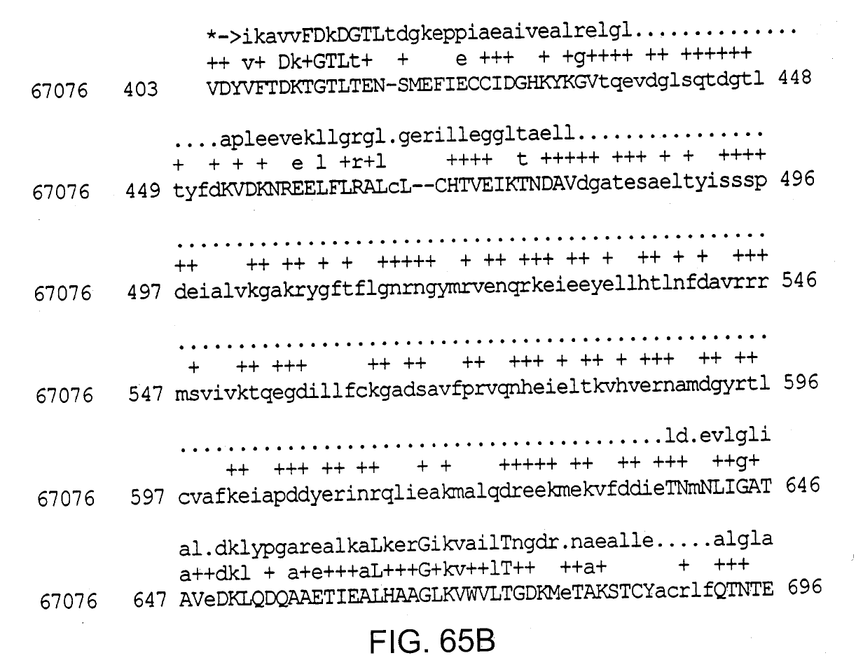

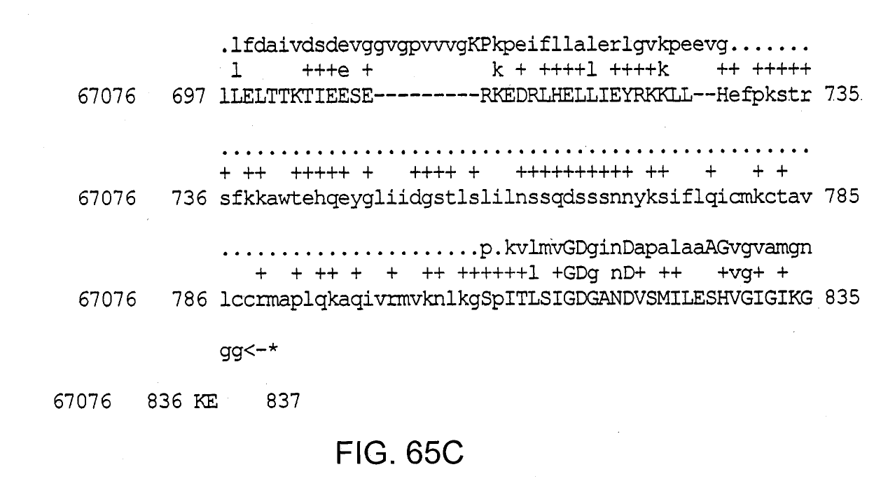

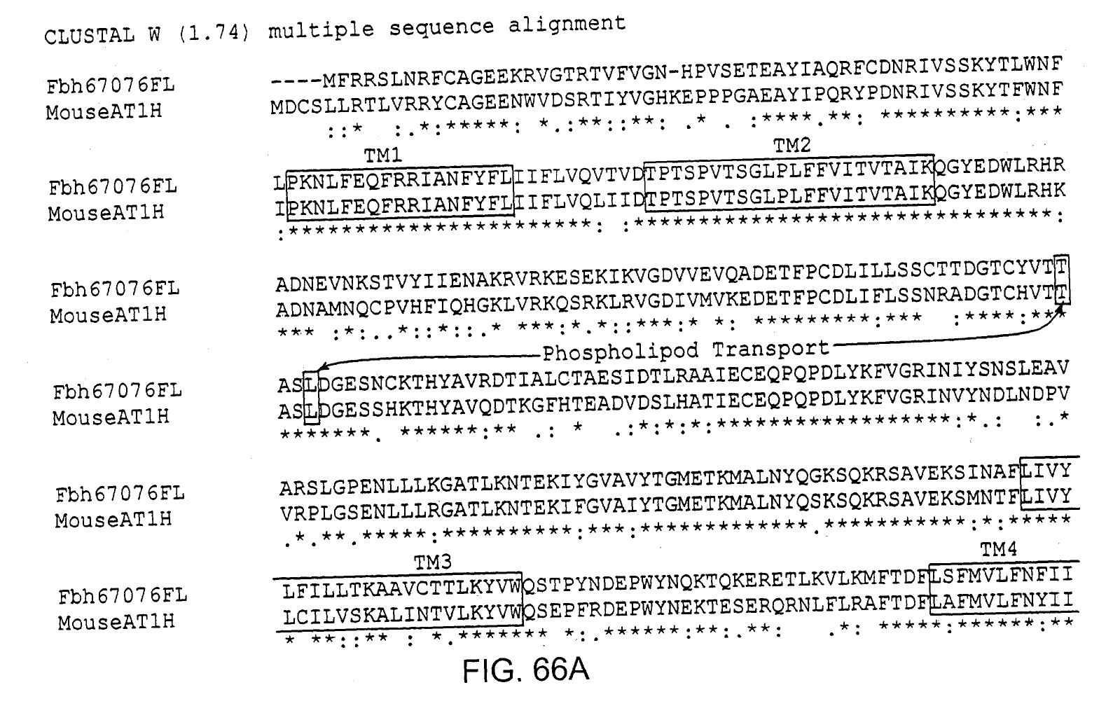

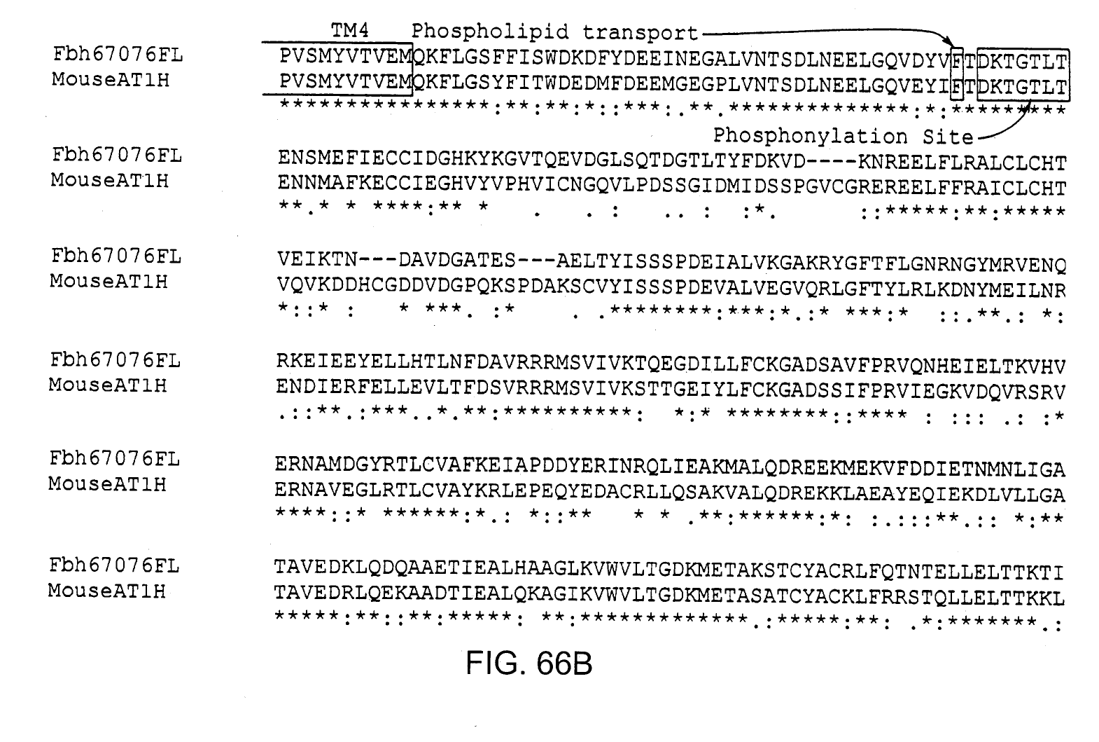

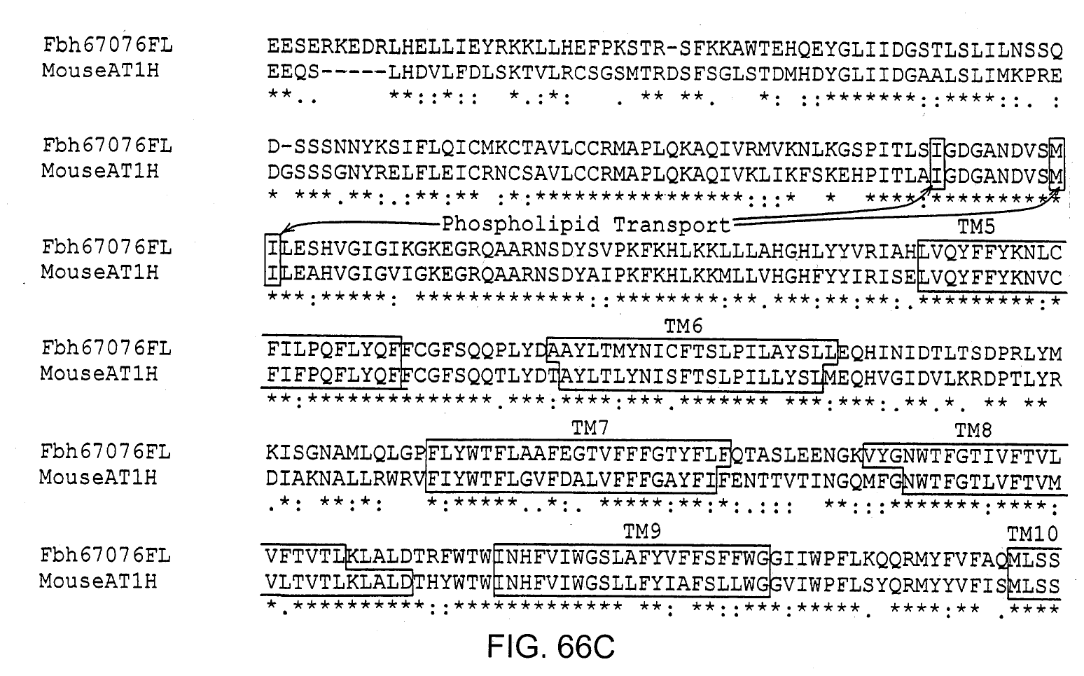

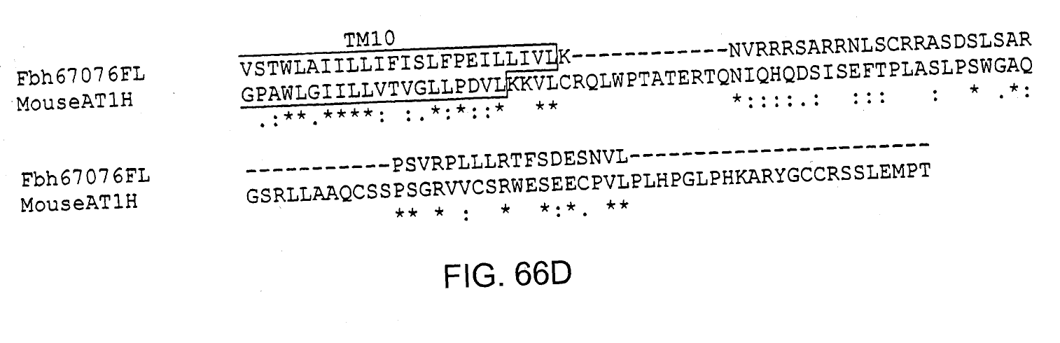

A MEMSAT analysis of the polypeptide sequence of SEQ ID NO:59 was also performed, predicting three potential transmembrane domains in the amino acid sequence of human 67076 (SEQ ID NO:59). However, a structural, hydrophobicity, and antigenicity analysis (FIG. 64) resulted in the identification of ten transmembrane domains. Accordingly, the 67076 protein of SEQ ID NO:59 is predicted to have at least ten transmembrane domains, identified as transmembrane (TM) domains 1 through 10, at about residues 57-77, 84-105, 292-313, 345-365, 863-883, 905-926, 956-977, 989-1009, 1021-1041, and 1060-1087.

A search using the polypeptide sequence of SEQ ID NO:59 was performed against the HMM database in PFAM resulting in the identification of a potential hydrolase domain in the amino acid sequence of human 67076 at about residues 403-837 of SEQ ID NO:59 (score=12.7).

A search of the amino acid sequence of human 67076 was also performed against the ProDom database resulting in the identification of several hydrolase domains and phosphorylation domains in the amino acid sequence of 67076 (SEQ ID NO:59).

The amino acid sequence of human 67102 was analyzed using the program PSORT. The results of this analysis predict that human 67102 may be localized to the endoplasmic reticulum.

Searches of the amino acid sequence of human 67102 were further performed against the Prosite database. These searches resulted in the identification in the amino acid sequence of human 67102 of a number of potential N-glycosylation sites at amino acid residues 29-32, 303-306, 1365-1368, and 1397-1400 of SEQ ID NO:62, a glycosaminoglycan attachment site at amino acid residues 526-529 of SEQ ID NO:62, a number of potential cAMP- and cGMP-dependent protein kinase phosphorylation sites at amino acid residues 38-41, 451-545, 635-638, and 777-780 of SEQ ID NO:62, a number of protein kinase C phosphorylation sites at amino acid residues 47-49, 78-80, 161-163, 240-242, 262-264, 280-282, 437-439, 500-502, 563-565, 633-635, 644-646, 695-697, 743-745, 774-776, 827-829, 1000-1002, 1360-1362, and 1371-1373 of SEQ ID NO:62, a number of potential casein kinase II phosphorylation sites at amino acid residues 20-23, 161-164, 176-179, 184-187, 199-202, 210-213, 232-235, 241-244, 262-265, 312-315, 345-348, 405-408, 442-445, 471-474, 477-480, 543-546, 621-624, 644-647, 670-673, 693-696, 727-730, 850-853, 866-869, 892-895, 977-980, 1074-1077, 1141-1144, 1199-1202, 1221-1224, 1339-1342, 1399-1402, and 1403-1406 of SEQ ID NO:62, two tyrosine kinase phosphorylation sites at amino acid residues 21-28 and 847-854 of SEQ ID NO:62, a number of potential N-myristoylation sites at amino acid residues 69-74, 341-346, 488-493, 510-515, 519-524, 525-530, 651-656, 703-708, 714-719, 901-906, 955-960, 992-997, 1070-1075, 1139-1144, 1229-1234, and 1261-1266 of SEQ ID NO:62, two potential amidation sites at amino acid residues 36-39 and 1371-1374 of SEQ ID NO:62, two ATP/GTP-binding site motif A (P-loop) at amino acid residues 996-1003 and 1364-1371, an E1-E2 ATPases phosphorylation site at amino acid residues 438-444 of SEQ ID NO:62, and a prokaryotic membrane lipoprotein lipid attachment site at amino acid residues 26-36 of SEQ ID NO:62.

A MEMSAT analysis of the polypeptide sequence of SEQ ID NO:62 was also performed, predicting ten potential transmembrane domains in the amino acid sequence of human 67102 (SEQ ID NO:62) at about residues 98-115, 122-140, 322-344, 366-390, 582-601, 752-770, 1145-1166, 1225-1246, 1253-1276, and 1298-1317.

A search using the polypeptide sequence of SEQ ID NO:62 was performed against the HMM database in PFAM resulting in the identification of a potential hydrolase domain in the amino acid sequence of human 67102 at about residues 432-1077 of SEQ ID NO:62 (score=1.5), and the identification of a potential DUF6 domain in the amino acid sequence of human 67102 at about residues 1127-1271 of SEQ ID NO:62 (score=−24.6).

A search of the amino acid sequence of human 67102 was also performed against the ProDom database resulting in the identification of several hydrolase domains and phosphorylation domains in the amino acid sequence of 667102 (SEQ ID NO:62).

The amino acid sequence of human 44181 was analyzed using the program PSORT. The results of this analysis predict that human 44181 may be localized to the endoplasmic reticulum.

Searches of the amino acid sequence of human 44181 were further performed against the Prosite database. These searches resulted in the identification in the amino acid sequence of human 44181 of a number of potential N-glycosylation sites at amino acid residues 331-334, 390-393, 449-452, 461-464, 477-480, 786-789, and 998-1001 of SEQ ID NO:65, a number of potential cAMP- and cGMP-dependent protein kinase phosphorylation sites at amino acid residues 577-580, 633-636, and 750-753 of SEQ ID NO:65, a number of protein kinase C phosphorylation sites at amino acid residues 46-48, 163-165, 276-278, 332-334, 406-408, 470-472, 574-576, 636-638, 957-959, 1014-1016, and 1102-1104 of SEQ ID NO:65, a number of potential casein kinase C phosphorylation sites at amino acid residues 115-118, 262-265, 280-283, 411-414, 473-476, 520-523, 527-530, 636-639, 678-681, 737-740, 906-909, 929-932, 1100-1103, 1154-1157, and 1170-1173 of SEQ ID NO:65, a potential tyrosine kinase phosphorylation site at amino acid residues 316-322 of SEQ ID NO:65, a number of potential N-myristoylation sites at amino acid residues 131-136, 596-601, 766-771, and 993-998 of SEQ ID NO:65, and an E1-E2 ATPases phosphorylation site at amino acid residues 407-413 of SEQ ID NO:65.

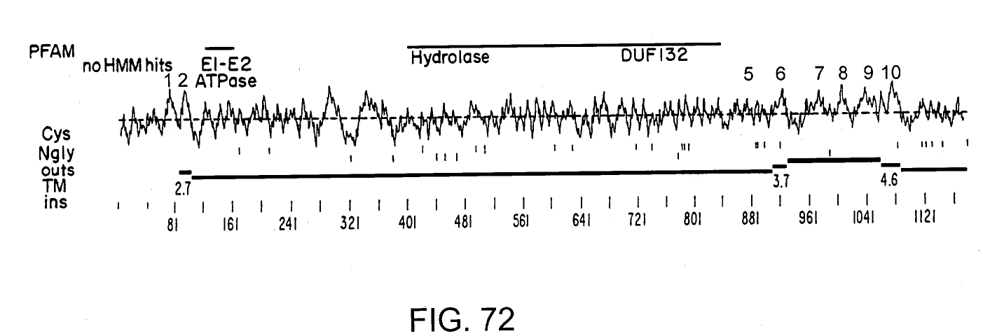

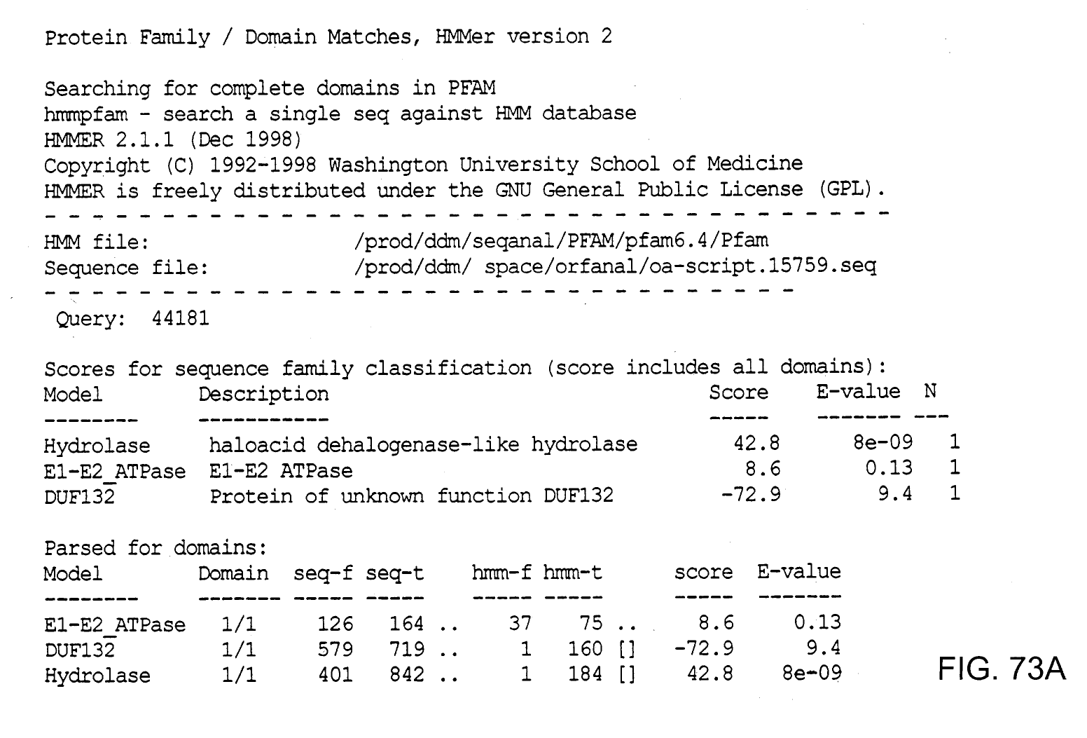

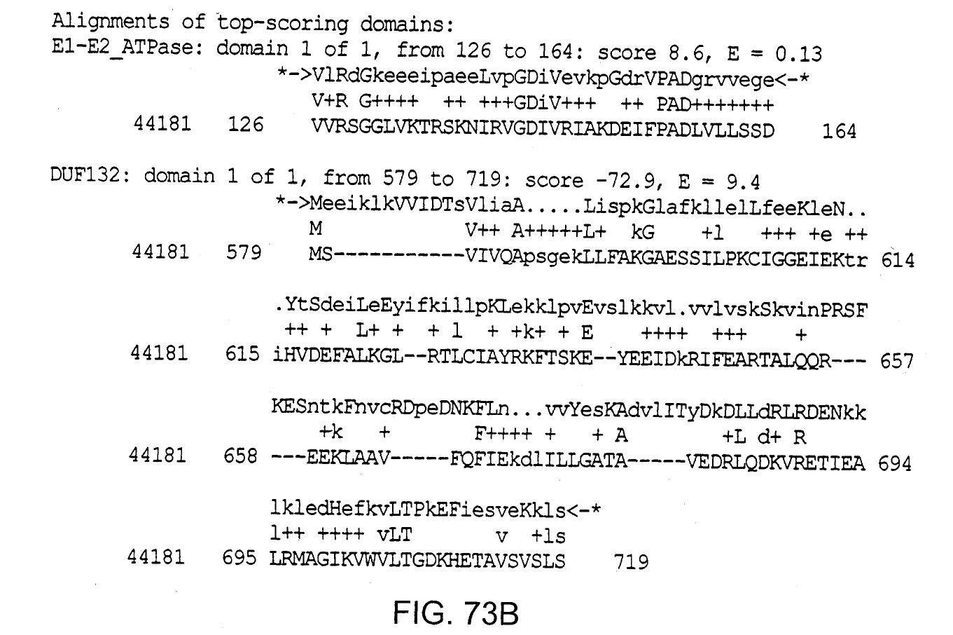

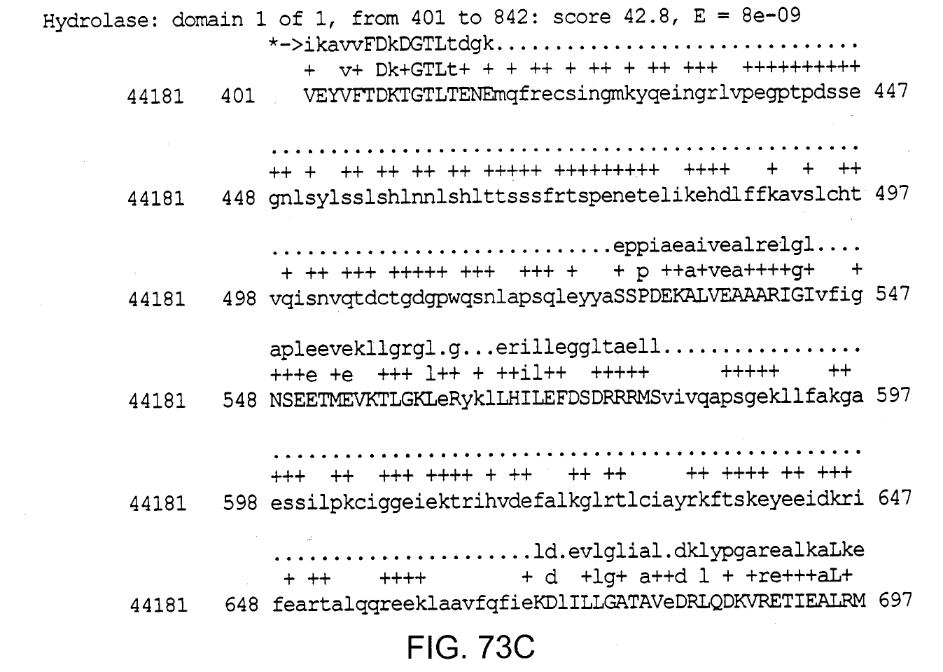

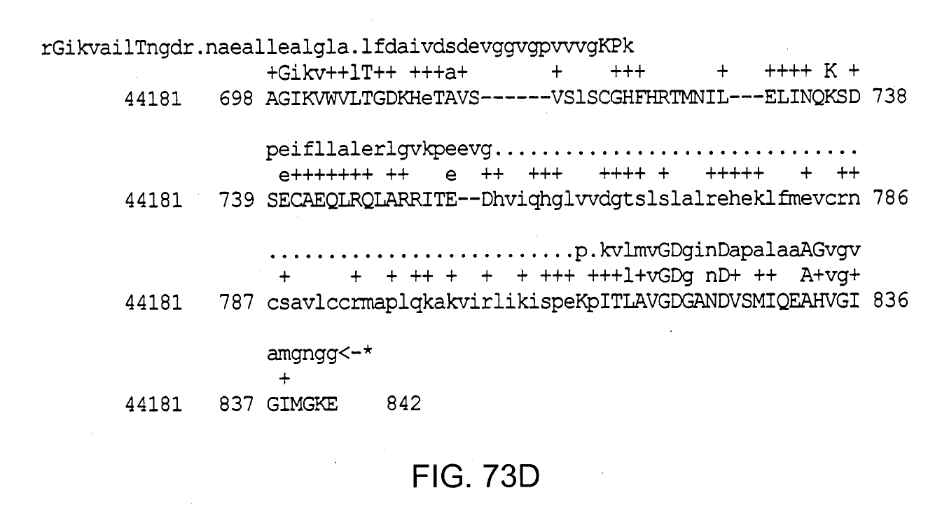

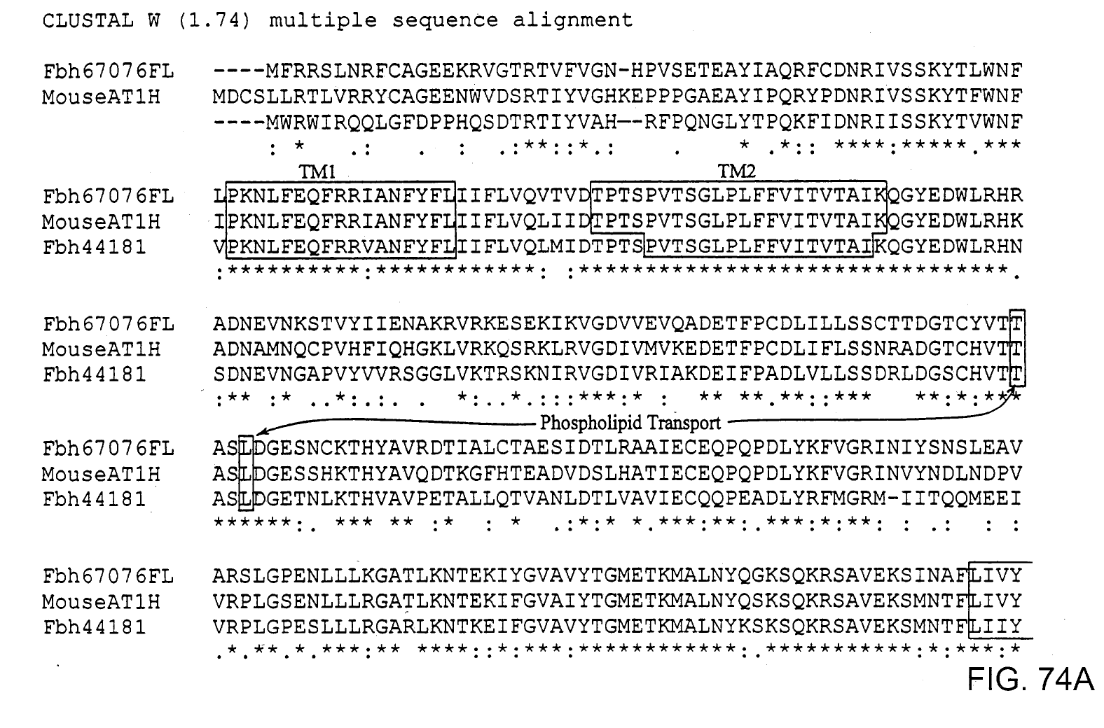

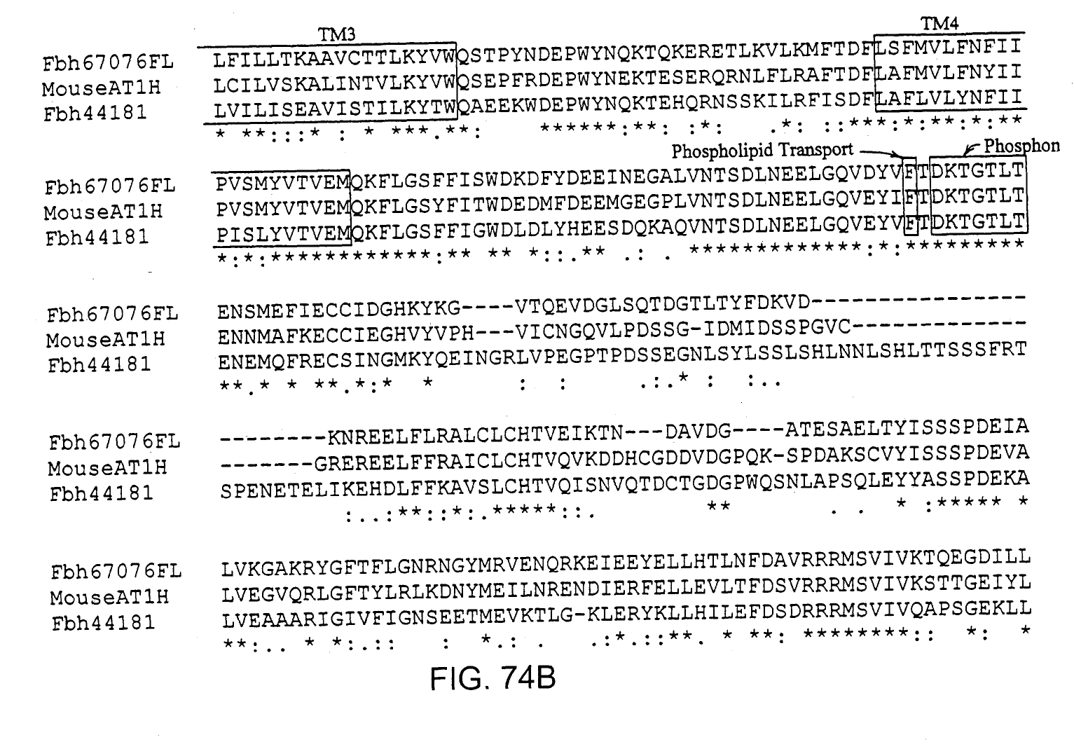

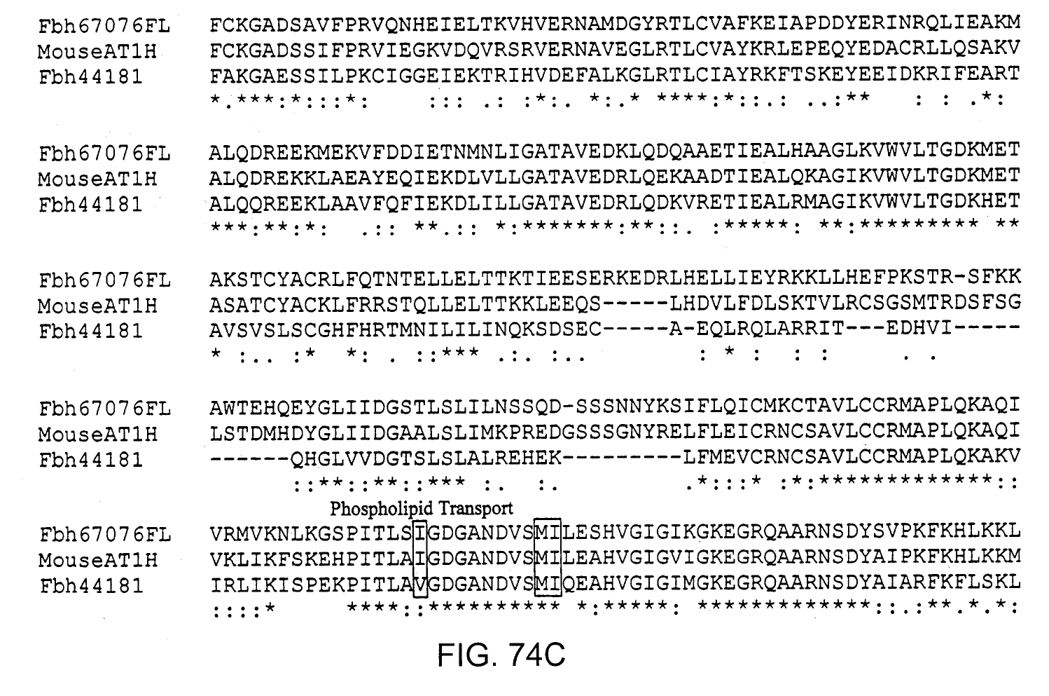

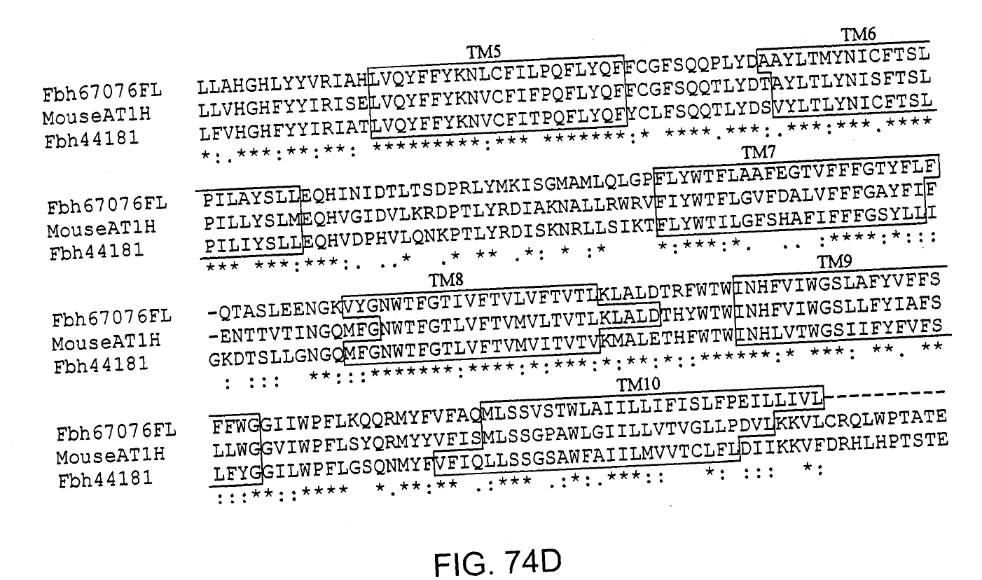

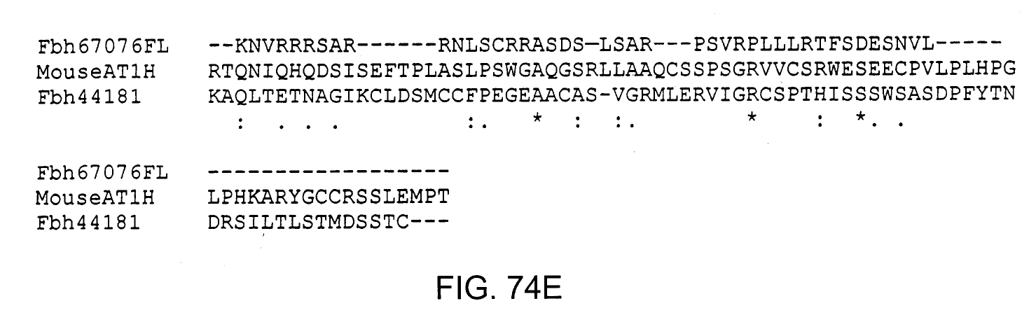

A MEMSAT analysis of the polypeptide sequence of SEQ ID NO:65 was also performed, predicting three potential transmembrane domains in the amino acid sequence of human 44181 (SEQ ID NO:65). However, a structural, hydrophobicity, and antigenicity analysis (FIG. 72) resulted in the identification of ten transmembrane domains. Accordingly, the 44181 protein (SEQ ID NO:65) is predicted to have at least ten transmembrane domains, which are identified as transmembrane (TM) domains 1 through 10, at about residues 56-72, 87-103, 290-311, 343-363, 878-898, 911-931, 961-982, 995-1015, 1027-1047, and 1062-1086.

A search using the polypeptide sequence of SEQ ID NO:65 was performed against the HMM database in PFAM resulting in the identification of a potential E1-E2 ATPase domain in the amino acid sequence of human 44181 at about residues 126-164 of SEQ ID NO:65 (score=8.6), the identification of a potential DUF132 domain in the amino acid sequence of human 44181 at about residues 579-719 of SEQ ID NO:65 (score=−72.9), and the identification of a potential hydrolase domain in the amino acid sequence of human 44181 at about residues 401-842 of SEQ ID NO:65 (score=42.8).

A search of the amino acid sequence of human 44181 was also performed against the ProDom database A search of the amino acid sequence of human 44181 was also performed against the ProDom database resulting in the identification of several hydrolase domains and phosphorylation domains in the amino acid sequence of 44181 (SEQ ID NO:65).

A Clustal W (1.74) alignment of the amino acid sequence of human 44181 (SEQ ID NO:65) and human potential phospholipid-transporting ATPase IR (ATIR; GenBank Accession No.:Q9Y2G3) revealed some sequence homology between 44181 and Accession No.:Q9Y2G3.

The amino acid sequence of human 67084FL was analyzed using the program PSORT. The results of this analysis predict that human 67084FL may be localized to the endoplasmic reticulum.

Searches of the amino acid sequence of human 67084FL were further performed against the Prosite database. These searches resulted in the identification in the amino acid sequence of human 67084FL of a number of potential N-glycosylation sites at amino acid residues 310-313, 464-467, and 529-532 of SEQ ID NO:68, a potential cAMP- and cGMP-dependent protein kinase phosphorylation site at amino acid residues 1071-1074 of SEQ ID NO:68, a number of protein kinase C phosphorylation sites 82-84, 168-170, 204-206, 301-303, 371-373, 415-417, 486-488, 585-587, 666-668, 744-746, 800-802, 813-815, 872-874, 957-959, and 1009-1011 of SEQ ID NO:68, a number of potential casein kinase II phosphorylation sites at amino acid residues 265-268, 301-304, 402-405, 422-425, 535-538, 596-599, 661-664, 686-689, and 745-748 of SEQ ID NO:68, a tyrosine kinase phosphorylation site at amino acid residues 813-816 of SEQ ID NO:68, a number of potential N-myristoylation sites at amino acid residues 292-297, 462-467, 568-573, 606-611, 824-829, 887-892, and 1026-1031 of SEQ ID NO:68, a potential amidation site at amino acid residues 813-816 of SEQ ID NO:68, a prokaryotic membrane lipoprotein lipid attachment site at amino acid residues 105-115, a leucine zipper pattern at amino acid residues 325-346, and an E1-E2 ATPases phosphorylation site at amino acid residues 416-422 of SEQ ID NO:68.

A MEMSAT analysis of the polypeptide sequence of SEQ ID NO:68 was also performed, predicting nine potential transmembrane domains in the amino acid sequence of human 67084FL (SEQ ID NO:68). However, a structural, hydrophobicity, and antigenicity analysis (FIG. 76) resulted in the identification of ten transmembrane domains. Accordingly, the 67084FL protein of SEQ ID NO:68 is predicted to have at least ten transmembrane domains, which are identified as transmembrane (TM) domains 1 through 10, at about residues 104-120, 124-144, 331-350, 357-374, 887-903, 912-931, 961-983, 990-1008, 1015-1035, and 1043-1067.

A search using the polypeptide sequence of SEQ ID NO:68 was performed against the HMM database in PFAM resulting in the identification of two potential E1-E2 ATPase in the amino acid sequence of human 67084FL at about residues 171-199 of SEQ ID NO:68 (score=3.0) and 277-305 of SEQ ID NO:68 (score=13.0), and a hydrolase domain at about residues 410-843 of SEQ ID NO:68 (score=19.2).

A search of the amino acid sequence of human 67084FL was also performed against the ProDom database resulting in the identification of several hydrolase domains, phosphorylation domains, and ATPase domains in the amino acid sequence of 67084FL (SEQ ID NO:68).

A Clustal W (1.74) alignment of the amino acid sequence of human 67084FL (SEQ ID NO:68) and human membrane transport protein (MTRP-1; GenBank Accession No.:Y71056, International Publication No. WO 2000/26245-A2) revealed some sequence homology between 67084FL and Accession No.: Y71056.

The amino acid sequence of human 67084alt was analyzed using the program PSORT. The results of this analysis predict that human 67084alt may be localized to the endoplasmic reticulum.

Searches of the amino acid sequence of human 67084alt were further performed against the Prosite database. These searches resulted in the identification in the amino acid sequence of human 67084alt of a number of potential N-glycosylation sites at amino acid residues 310-313, 464-467, and 529-532 of SEQ ID NO:71, a potential cAMP- and cGMP-dependent protein kinase phosphorylation site at amino acid residues 1083-1086, a number of protein kinase C phosphorylation sites at amino acid residues 82-84, 168-170, 204-2-6, 301-303, 371-373, 415-417, 486-488, 585-587, 666-668, 744-746, 800-802, 813-815, 872-874, 957-959, and 1009-1011 of SEQ ID NO:71, a number of potential casein kinase II phosphorylation sites at amino acid residues 265-268, 301-304, 402-405, 422-445, 535-538, 596-599, 661-664, 686-689, and 745-748 of SEQ ID NO:71, a tyrosine kinase phosphorylation site at amino acid residues 641-648, a number of potential N-myristoylation sites at amino acid residues 292-297, 462-467, 568-573, 606-611, 824-829, 887-892, and 1026-1031 of SEQ ID NO:71, a potential amidation site at amino acid residues 813-816 of SEQ ID NO:71, a potential prokaryotic membrane lipoprotein lipid attachment site at amino acid residues 105-115 of SEQ ID NO:71, a leucine zipper pattern at amino acid residues 325-346 of SEQ ID NO:71, and an E1-E2 ATPases phosphorylation site at amino acid residues 416-422 of SEQ ID NO:71.

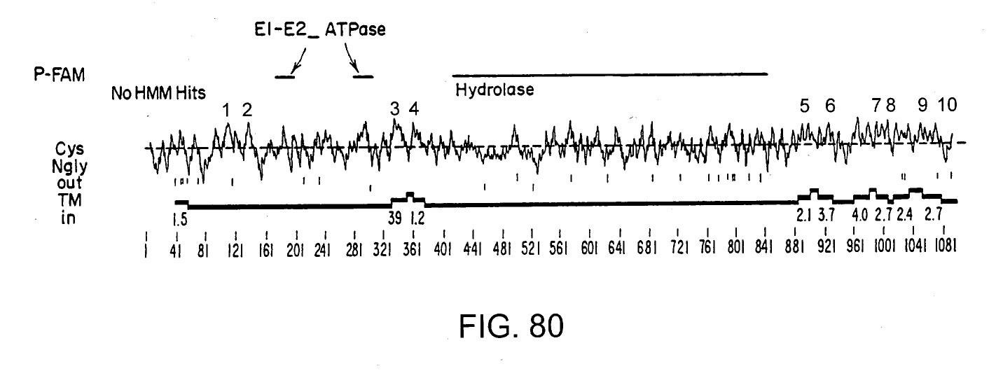

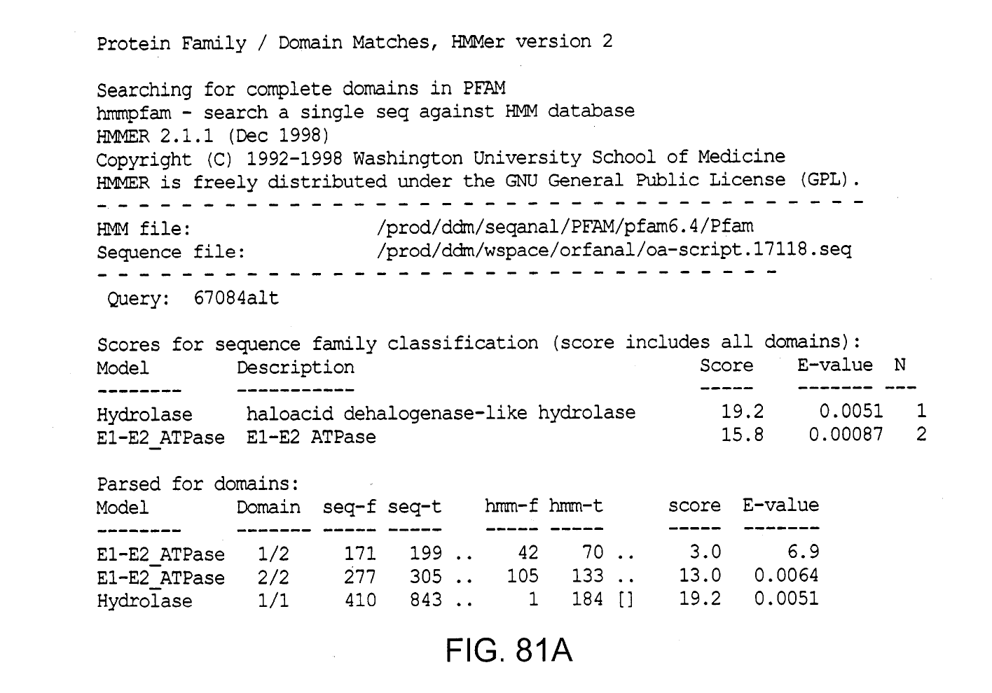

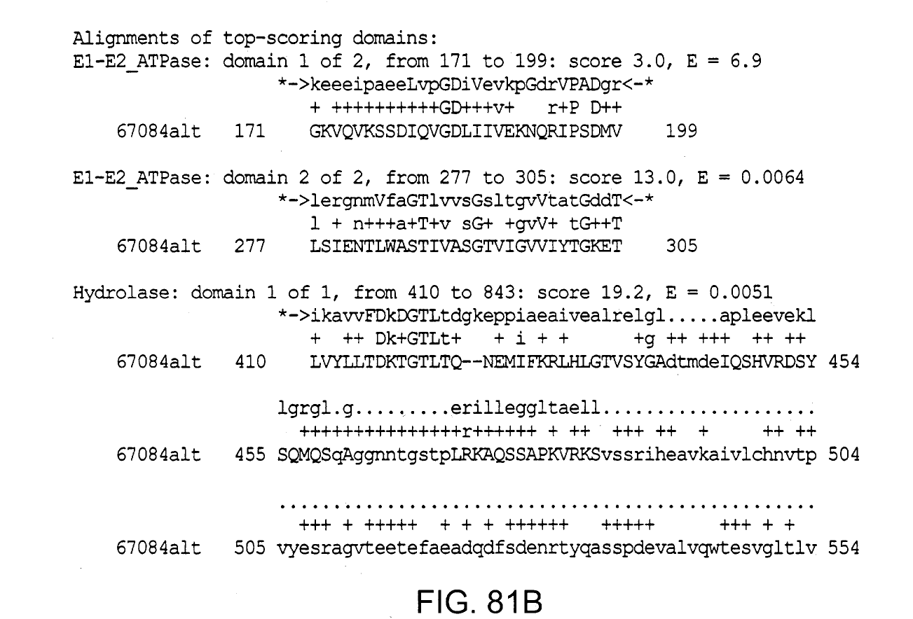

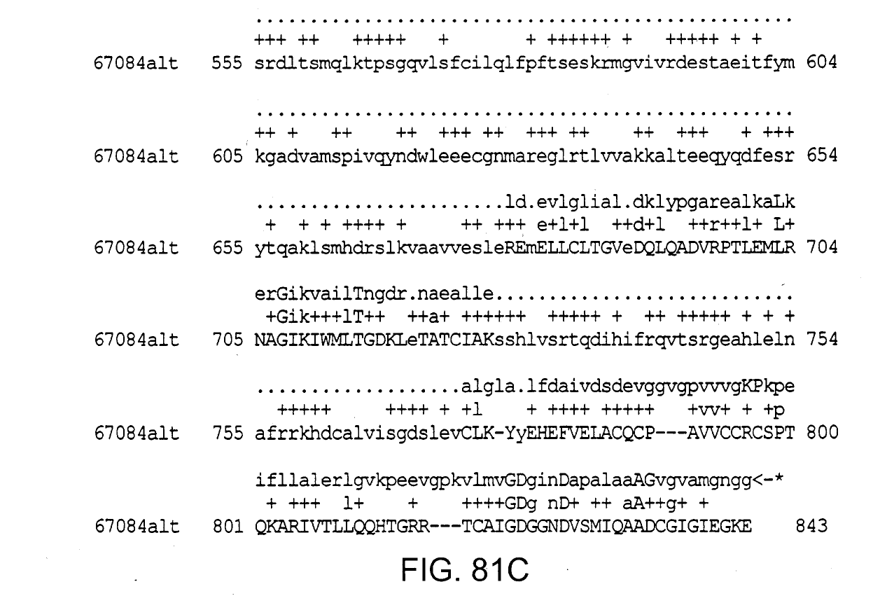

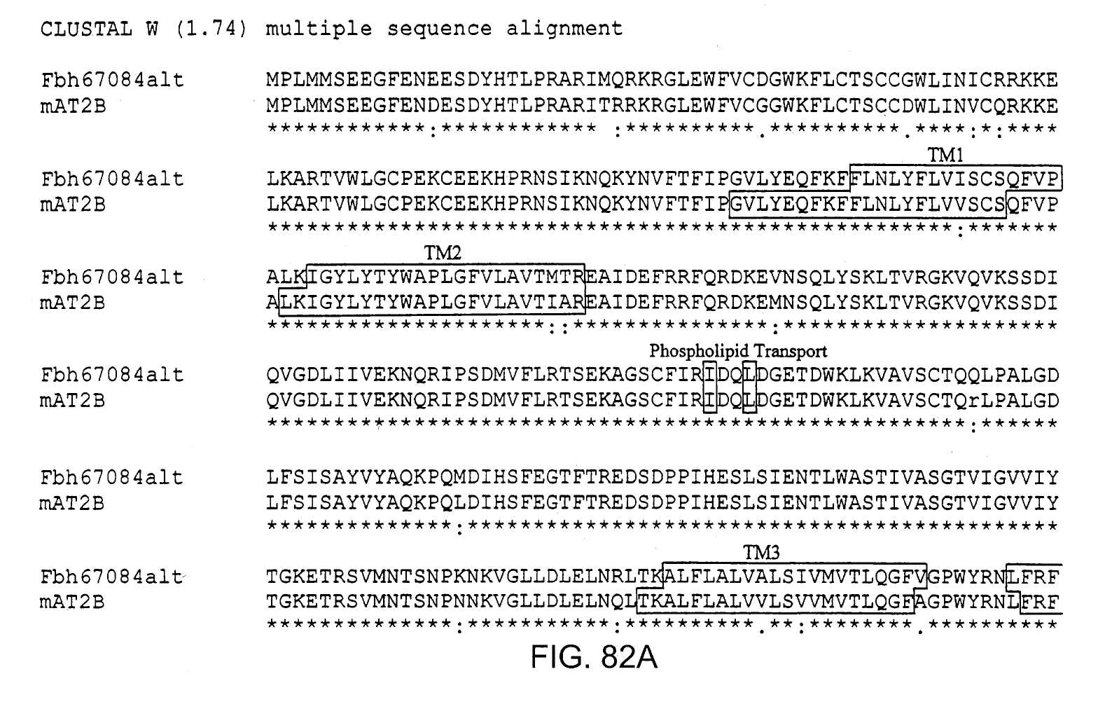

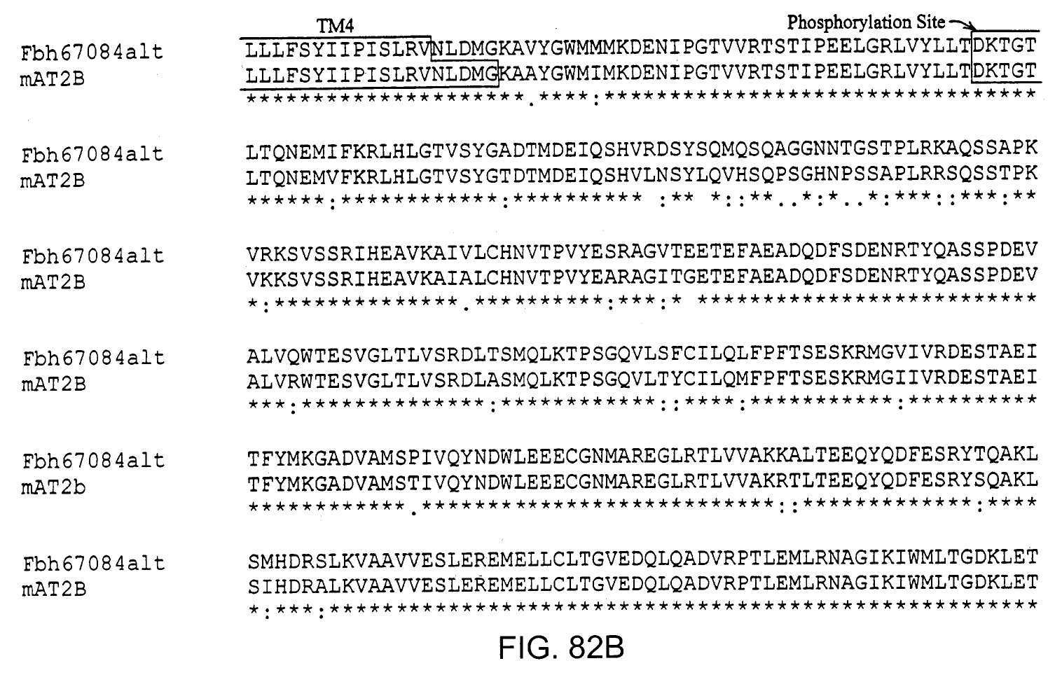

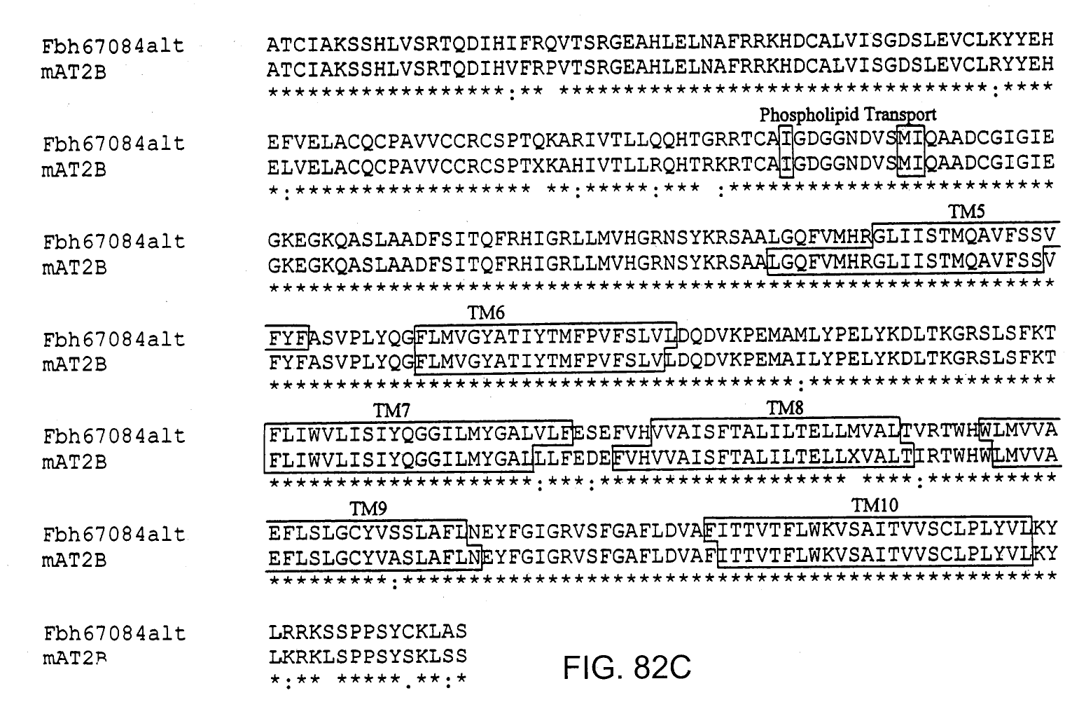

A MEMSAT analysis of the polypeptide sequence of SEQ ID NO:71 was also performed, predicting nine potential transmembrane domains in the amino acid sequence of human 67084alt (SEQ ID NO:71). However, a structural, hydrophobicity, and antigenicity analysis (FIG. 80) resulted in the identification of ten transmembrane domains. Accordingly, the 67084alt protein of SEQ ID NO:71 is predicted to have at least ten transmembrane domains, which are identified as transmembrane (TM) domains 1 through 10, at about residues 104-120, 124-144, 331-350, 357-374, 887-903, 912-931, 961-983, 990-1008, 1015-1035, and 1043-1067.

A search using the polypeptide sequence of SEQ ID NO:71 was performed against the HMM database in PFAM resulting in the identification of two potential E1-E2 ATPase in the amino acid sequence of human 67084alt at about residues 42-70 of SEQ ID NO:71 (score=3.0) and 105-133 of SEQ ID NO:71 (score=13.0), and a potential hydrolase domain at about amino acid residues 410-843 of SEQ ID NO:71 (score=19.2).

A search of the amino acid sequence of human 67084alt was also performed against the ProDom database resulting in the identification of several hydrolase domains, phosphorylation domains, and ATPase domains in the amino acid sequence of 67084alt (SEQ ID NO:71).

A Clustal W (1.74) alignment of the amino acid sequence of human 67084alt (SEQ ID NO:59) and human membrane transport protein (MTRP-1; GenBank Accession No.:Y71056, International Publication No. WO 2000/26245-A2) revealed some sequence homology between 67084alt and Accession No.: Y71056.

### Example 2

### Tissue Expression of Human 8099, 46455, 54414, 53763, 67076, 67102, 44181, Full Length 67084 (67084FL), and 67084alt mRNA

Tissue Distribution of Human 8099, 46455, 54414, 53763, 67076, 67102, 44181, 67084FL, and 67084alt mRNA Using Taqman™ Analysis

This example describes the tissue distribution of human 8099, 46455, 54414, 53763, 67076, 67102, 44181, 67084FL, or 67084alt mRNA in a variety of cells and tissues, as determined using the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., lung, ovary, colon, and breast normal and tumor samples, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

Tissue Distribution of Human 8099

A human tissue panel was tested revealing highest expression of human 8099 mRNA in congestive heart failure (CHF) heart, normal prostate, and brain (see Table 1, below).

TABLE 1

| | | β 2 | | |
|---|---|---|---|---|
| Tissue Type | Mean | Mean | ∂∂ Ct | Expression |
| Artery normal | 30.83 | 22.31 | 8.52 | 2.7241 |
| Aorta diseased | 32.77 | 22.32 | 10.45 | 0.7149 |
| Vein normal | 29.41 | 20.23 | 9.18 | 1.724 |
| Coronary SMC | 31.2 | 20.91 | 10.3 | 0.7932 |
| HUVEC | 32.16 | 21.38 | 10.78 | 0.5687 |
| Hemangioma | 32.86 | 19.66 | 13.21 | 0.1059 |
| Heart normal | 28.05 | 20.43 | 7.62 | 5.0834 |
| Heart CHF | 26.98 | 20.68 | 6.3 | 12.6914 |
| Kidney | 27.76 | 20.45 | 7.3 | 6.3238 |
| Skeletal Muscle | 29.7 | 22.17 | 7.53 | 5.4294 |
| Adipose normal | 34.16 | 20.59 | 13.56 | 0.0828 |
| Pancreas | 33.23 | 22.29 | 10.94 | 0.5108 |
| primary osteoblasts | 32 | 20.61 | 11.39 | 0.3726 |
| Osteoclasts (diff) | 30.9 | 17.55 | 13.35 | 0.0958 |
| Skin normal | 34.12 | 22.45 | 11.68 | 0.3058 |
| Spinal cord normal | 31.93 | 21.07 | 10.87 | 0.5362 |
| Brain Cortex normal | 28.4 | 22.34 | 6.06 | 14.9885 |
| Brain Hypothalamus normal | 29.68 | 22.35 | 7.34 | 6.1936 |
| Nerve | 32.96 | 22.25 | 10.72 | 0.5949 |
| DRG (Dorsal Root Ganglion) | 30.81 | 22.15 | 8.65 | 2.4808 |
| Breast normal | 31.91 | 21.14 | 10.77 | 0.5747 |
| Breast tumor | 32.73 | 20.93 | 11.81 | 0.2785 |
| Ovary normal | 30.41 | 19.82 | 10.6 | 0.6465 |
| Ovary Tumor | 28.36 | 19.06 | 9.31 | 1.5755 |
| Prostate Normal | 27.29 | 19.77 | 7.52 | 5.4482 |

Tissue Distribution of Human 46455

A human vessel and tissue panel was tested revealing highest expression of human 46455 mRNA in human umbilical vein endothelial cells (HUVEC), erythroid cells, normal artery, megakaryocytes, kidney, and CHF heart. 46455 was expressed at higher levels in lung tumor, breast tumor, and colon tumor versus normal lung, breast and colon tissues, indicating a possible role for 46455 in cellular proliferation disorders (see Table 2, below).

TABLE 2

| Tissue Type | Mean | β 2 Mean | ∂∂ Ct | Expression |
|---|---|---|---|---|
| Artery normal | 28.05 | 24.09 | 2.67 | 157.6722 |
| Aorta diseased | 28.75 | 23.66 | 3.79 | 72.0429 |
| Vein normal | 27.75 | 21.72 | 4.75 | 37.1627 |
| Coronary SMC | 28.2 | 25.12 | 1.78 | 290.176 |
| HUVEC | 24.18 | 22.59 | 0.29 | 817.9021 |
| Hemangioma | 25.15 | 20.98 | 2.88 | 135.8419 |
| Heart normal | 26.44 | 21.82 | 3.33 | 99.4421 |
| Heart CHF | 25.54 | 21.09 | 3.15 | 112.6563 |
| Kidney | 25.98 | 21.49 | 3.2 | 108.8188 |
| Skeletal Muscle | 28.22 | 24.14 | 2.79 | 144.586 |
| Adipose normal | 28.38 | 22.21 | 4.88 | 33.9605 |
| Pancreas | 27.91 | 23.22 | 3.4 | 94.4045 |
| primary osteoblasts | 27.11 | 21.85 | 3.97 | 63.8133 |
| Osteoclasts (diff) | 23.64 | 18.8 | 3.55 | 85.3775 |
| Skin normal | 28.43 | 23.27 | 3.88 | 68.1567 |
| Spinal cord normal | 26.88 | 22.12 | 3.47 | 90.2456 |
| Brain Cortex normal | 26.42 | 23.4 | 1.73 | 301.452 |
| Brain Hypothalamus normal | 28.1 | 23.55 | 3.26 | 104.386 |
| Nerve | 28.59 | 23.88 | 3.43 | 92.7827 |
| DRG (Dorsal Root Ganglion) | 28.33 | 23.76 | 3.28 | 102.9489 |
| Breast normal | 27.31 | 22.32 | 3.7 | 76.9465 |
| Breast tumor | 26.47 | 22.11 | 3.07 | 119.0797 |
| Ovary normal | 26.59 | 22.16 | 3.13 | 113.8337 |
| Ovary Tumor | 28.47 | 21.84 | 5.33 | 24.8605 |
| Prostate Normal | 27.09 | 21.68 | 4.12 | 57.5117 |
| Prostate Tumor | 26.51 | 21.58 | 3.64 | 80.2141 |
| Salivary glands | 27.16 | 20.81 | 5.07 | 29.8733 |
| Colon normal | 26.3 | 20 | 5 | 31.1419 |
| Colon Tumor | 25.09 | 20.52 | 3.29 | 102.5927 |
| Lung normal | 26.02 | 19.75 | 4.98 | 31.6862 |
| Lung tumor | 25.09 | 21.31 | 2.48 | 178.6243 |
| Lung COPD | 25.26 | 19.71 | 4.26 | 52.193 |
| Colon IBD | 26.3 | 18.91 | 6.1 | 14.5786 |
| Liver normal | 27.66 | 21.8 | 4.57 | 42.101 |
| Liver fibrosis | 29.31 | 24.09 | 3.92 | 65.8351 |
| Spleen normal | 27.41 | 21.41 | 4.71 | 38.2075 |
| Tonsil normal | 25.23 | 19.32 | 4.63 | 40.5262 |
| Lymph node normal | 26.15 | 20.35 | 4.51 | 43.8889 |
| Small intestine normal | 28.23 | 21.73 | 5.22 | 26.8302 |
| Skin-Decubitus | 27.18 | 22.82 | 3.06 | 119.908 |
| Synovium | 28 | 21.12 | 5.59 | 20.6889 |
| BM-MNC | 26.13 | 19.32 | 5.51 | 21.9445 |
| Activated PBMC | 25.2 | 17.95 | 5.96 | 16.12 |
| Neutrophils | 24.45 | 19.5 | 3.65 | 79.66 |
| Megakaryocytes | 22.5 | 18.95 | 2.26 | 208.772 |
| Erythroid | 24.2 | 21.69 | 1.23 | 427.7975 |

Tissue Distribution of Human 53763

A human vessel and tissue panel was tested revealing highest expression of human 53763 mRNA in normal brain cortex, normal hypothalamus, prostate tumor, normal prostate, dorsal root ganglion, and normal breast tissue (see Table 3, below).

TABLE 3

| Tissue Type | Mean | β 2 Mean | ∂∂ Ct | Expression |
|---|---|---|---|---|
| Artery normal | 40 | 22.41 | 16.07 | 0 |
| Aorta diseased | 40 | 22.05 | 16.44 | 0 |
| Vein normal | 40 | 19.75 | 18.74 | 0 |
| Coronary SMC | 35.16 | 21.86 | 11.79 | 0 |
| HUVEC | 40 | 20.41 | 18.08 | 0 |
| Hemangioma | 40 | 18.52 | 19.96 | 0 |
| Heart normal | 39.43 | 19.55 | 18.37 | 0 |
| Heart CHF | 40 | 18.98 | 19.5 | 0 |
| Kidney | 39.44 | 19.76 | 18.16 | 0 |
| Skeletal Muscle | 38.97 | 21.57 | 15.89 | 0 |
| Adipose normal | 40 | 20.09 | 18.4 | 0 |
| Pancreas | 38.91 | 20.84 | 16.56 | 0 |
| primary osteoblasts | 40 | 19.87 | 18.61 | 0 |
| Osteoclasts (diff) | 40 | 17.09 | 21.4 | 0 |
| Skin normal | 39.59 | 21.22 | 16.86 | 0 |
| Spinal cord normal | 31.72 | 20.14 | 10.07 | 0.9303 |
| Brain Cortex normal | 23.07 | 21.56 | 0.01 | 996.5403 |
| Brain Hypothalamus normal | 26.15 | 20.98 | 3.65 | 79.3844 |
| Nerve | 39.08 | 21.23 | 16.33 | 0 |
| DRG (Dorsal Root Ganglion) | 31.66 | 21.3 | 8.86 | 2.1596 |
| Breast normal | 27.25 | 20.41 | 5.33 | 24.9468 |
| Breast tumor | 40 | 20.02 | 18.46 | 0 |
| Ovary normal | 40 | 19.66 | 18.83 | 0 |
| Ovary Tumor | 40 | 19.7 | 18.79 | 0 |
| Prostate Normal | 29.68 | 19.32 | 8.85 | 2.1671 |
| Prostate Tumor | 28.14 | 19.95 | 6.67 | 9.8204 |
| Salivary glands | 40 | 18.97 | 19.52 | 0 |
| Colon normal | 39.09 | 17.8 | 19.78 | 0 |
| Colon Tumor | 40 | 18.63 | 19.86 | 0 |
| Lung normal | 40 | 17.49 | 21 | 0 |
| Lung tumor | 39.66 | 19.81 | 18.34 | 0 |
| Lung COPD | 40 | 17.97 | 20.52 | 0 |
| Colon IBD | 40 | 17.3 | 21.18 | 0 |
| Liver normal | 40 | 19.57 | 18.91 | 0 |
| Liver fibrosis | 40 | 21.34 | 17.15 | 0 |
| Spleen normal | 40 | 19.27 | 19.22 | 0 |
| Tonsil normal | 33.5 | 16.75 | 15.24 | 0.0258 |
| Lymph node normal | 38.61 | 18.4 | 18.7 | 0 |
| Small intestine normal | 36.56 | 19.96 | 15.08 | 0 |
| Skin-Decubitus | 39.43 | 20.41 | 17.51 | 0 |
| Synovium | 40 | 19.32 | 19.16 | 0 |
| BM-MNC | 40 | 18.21 | 20.27 | 0 |
| Activated PBMC | 38.88 | 17.5 | 19.88 | 0 |
| Neutrophils | 40 | 18.38 | 20.11 | 0 |
| Megakaryocytes | 40 | 18.09 | 20.39 | 0 |
| Erythroid | 40 | 21.23 | 17.25 | 0 |

Tissue Distribution of Human 67076

A human vessel panel was tested revealing highest expression of human 67076 mRNA in nornal aorta, diseased artery, and static HUVEC (see Table 4, below).

TABLE 4

| Tissue Type | Mean | β 2 Mean | ∂∂ Ct | Expression |
|---|---|---|---|---|
| Aortic SMC | 25.58 | 21.16 | 4.42 | 46.8762 |
| Coronary SMC | 29.11 | 24.36 | 4.76 | 36.906 |
| Huvec Static | 23.55 | 20.59 | 2.96 | 128.0696 |
| Huvec LSS | 23.41 | 20.06 | 3.35 | 98.073 |
| H/Adipose/MET 8 | 27.7 | 20.51 | 7.18 | 6.8723 |
| H/Artery/Normal/Carotid/CLN | 26.82 | 19.34 | 7.48 | 5.6014 |
| 595 | | | | |
| H/Artery/Normal/Carotid/CLN | 28.79 | 20.41 | 8.37 | 3.0226 |
| 598 | | | | |
| H/Artery/normal/NDR 352 | 29.41 | 21.68 | 7.73 | 4.7102 |
| H/IM Artery/Normal/AMC 73 | 32.65 | 23.77 | 8.88 | 2.1152 |
| H/Muscular Artery/Normal/ | 29.2 | 23.34 | 5.87 | 17.1577 |
| AMC 236 | | | | |
| H/Muscular Artery/Normal/ | 29.68 | 22.56 | 7.13 | 7.1393 |
| AMC 254/ | | | | |
| H/Muscular Artery/Normal/ | 29.63 | 22.25 | 7.37 | 6.0452 |
| AMC 259 | | | | |
| H/Muscular Artery/Normal/ | 30.12 | 22.67 | 7.45 | 5.7191 |
| AMC 261 | | | | |
| H/Muscular Artery/Normal/ | 30.2 | 24.2 | 6 | 15.6792 |
| AMC 275 | | | | |
| H/Aorta/Diseased/PIT 732 | 30.73 | 22.36 | 8.38 | 3.0121 |
| H/Aorta/Diseased/PIT 710 | 29.6 | 23.14 | 6.46 | 11.3199 |
| H/Aorta/Diseased/PIT 711 | 29.35 | 22.63 | 6.72 | 9.4531 |
| H/Aorta/Diseased/PIT 712 | 28.77 | 22.02 | 6.75 | 9.2585 |
| H/Artery/Diseased/iliac/NDR | 26.11 | 19.41 | 6.71 | 9.585 |
| 753 | | | | |
| H/Artery/Diseased/Tibial/PIT | 29.82 | 20.34 | 9.47 | 1.4101 |
| 679 | | | | |
| H/Vein/Normal/SaphenousAMC | 31.66 | 21.07 | 10.59 | 0.6488 |
| 107 | | | | |
| H/Vein/Normal/NDR 239 | 33.13 | 21.65 | 11.49 | 0.3477 |
| H/Vein/Normal/Saphenous/NDR | 29.71 | 20.59 | 9.12 | 1.7972 |
| 237 | | | | |
| H/Vein/Normal/PIT 1010 | 28.34 | 22.05 | 6.3 | 12.6914 |
| H/Vein/Normal/AMC 191 | 28.64 | 22.15 | 6.49 | 11.164 |
| H/Vein/Normal/AMC 130 | 27.41 | 21.27 | 6.14 | 14.1309 |
| H/Vein/Normal/AMC 188 | 30.56 | 24.09 | 6.46 | 11.3199 |
| H/Vein/Normal/AMC 196 | 29.89 | 20.93 | 8.96 | 2.008 |
| H/Vein/Normal/AMC 211 | 32.55 | 23.52 | 9.03 | 1.9196 |
| H/Vein/Normal/AMC 214 | 30.93 | 22.99 | 7.95 | 4.058 |
| M/Artery/Diseased/CAR 1174 | 24.56 | 23.05 | 1.5 | 352.3302 |
| M/Artery/Diseased/CAR 1175 | 24.98 | 19.89 | 5.09 | 29.2585 |
| M/Aorta/Normal/PRI 286 | 25.52 | 18.68 | 6.84 | 8.7288 |
| M/Artery/Normal/PRI 324 | 25.13 | 20.65 | 4.48 | 44.8111 |
| M/Aorta/Normal/PRI 264 | 24.14 | 24.74 | −0.6 | 1515.7166 |
| M/Artery/Normal/PRI 320 | 24.93 | 20.29 | 4.64 | 40.1071 |
| M/Vein/Normal/PRI 328 | 26.67 | 20.04 | 6.63 | 10.0965 |
| HUVEC Vehicle | 26.64 | 21 | 5.63 | 20.1232 |
| HUVEC Mev | 25.54 | 20.3 | 5.25 | 26.3692 |
| HAEC Vehicle | 25.7 | 20.66 | 5.04 | 30.2903 |
| HAEC Mev | 27.84 | 22.41 | 5.43 | 23.1957 |

Tissue Distribution of Human 67102

A human tissue panel was tested revealing highest expression of human 67102 mRNA in normal kidney tissue and diseased artery (see Table 5, below).

TABLE 5

| Tissue Type | Mean | β 2 Mean | ∂∂ Ct | Expression |
|---|---|---|---|---|
| Aortic SMC | 28.34 | 21.88 | 6.47 | 11.2807 |
| Coronary SMC | 29.98 | 23.11 | 6.88 | 8.5196 |
| Huvec Static | 27.55 | 21.41 | 6.14 | 14.18 |
| Huvec LSS | 27.72 | 21.12 | 6.59 | 10.3444 |
| H/Adipose/MET 8 | 30.56 | 20.57 | 9.99 | 0.9834 |
| H/Artery/Normal/Carotid/CLN | 31.42 | 20.3 | 11.13 | 0.4478 |
| 595 | | | | |
| H/Artery/Normal/Carotid/CLN | 32.23 | 21.69 | 10.54 | 0.6717 |
| 598 | | | | |
| H/Artery/normal/NDR 352 | 31.34 | 22.44 | 8.9 | 2.0933 |
| H/IM Artery/Normal/AMC 73 | 33.46 | 23.98 | 9.48 | 1.4003 |
| H/Muscular Artery/Normal/ | 30.48 | 23.52 | 6.96 | 8.0321 |
| AMC 236 | | | | |
| H/Muscular Artery/Normal/ | 33.9 | 24.07 | 9.82 | 1.1025 |
| AMC 247 | | | | |
| H/Muscular Artery/Normal/ | 31.12 | 23.43 | 7.68 | 4.8594 |
| AMC 254/ | | | | |
| H/Muscular Artery/Normal/ | 30.47 | 23.07 | 7.4 | 5.9208 |
| AMC 259 | | | | |
| H/Muscular Artery/Normal/ | 31.32 | 22.92 | 8.4 | 2.9501 |
| AMC 261 | | | | |
| H/Muscular Artery/Normal/ | 31.31 | 24.78 | 6.53 | 10.8212 |
| AMC 275 | | | | |
| H/Aorta/Diseased/PIT 732 | 31.73 | 22.76 | 8.97 | 1.9942 |
| H/Aorta/Diseased/PIT 710 | 30.33 | 23.36 | 6.97 | 7.9767 |
| H/Aorta/Diseased/PIT 711 | 31.02 | 23.3 | 7.72 | 4.7265 |
| H/Aorta/Diseased/PIT 712 | 30.57 | 22.71 | 7.86 | 4.3043 |
| H/Artery/Diseased/iliac/NDR | 27.22 | 20.07 | 7.15 | 7.041 |
| 753 | | | | |
| H/Artery/Diseased/Tibial/PIT | 32 | 21.19 | 10.81 | 0.557 |
| 679 | | | | |
| H/Vein/Normal/SaphenousAMC | 31.57 | 22.08 | 9.49 | 1.3859 |
| 107 | | | | |
| H/Vein/Normal/NDR 239 | 33.44 | 22.16 | 11.28 | 0.4021 |
| H/Vein/Normal/Saphenous/NDR | 31.32 | 21.01 | 10.31 | 0.7877 |
| 237 | | | | |
| H/Vein/Normal/PIT 1010 | 29.86 | 22.36 | 7.5 | 5.5243 |
| H/Vein/Normal/AMC 191 | 30.36 | 22.53 | 7.84 | 4.3796 |
| H/Vein/Normal/AMC 130 | 30.08 | 22.32 | 7.75 | 4.6293 |
| H/Vein/Normal/AMC 188 | 32.93 | 25.01 | 7.92 | 4.129 |
| H/Vein/Normal/AMC 196 | 32.24 | 21.61 | 10.64 | 0.6288 |
| H/Vein/Normal/AMC 211 | 36.16 | 23.51 | 12.65 | 0 |
| H/Vein/Normal/AMC 214 | 35.59 | 24 | 11.6 | 0 |
| M/Artery/Diseased/CAR 1175 | 29.73 | 21.84 | 7.89 | 4.2011 |
| M/Aorta/Normal/543 | 34.84 | 29.17 | 5.67 | 19.6408 |
| M/Artery/Diseased/CAR 1174 | 31.11 | 26.59 | 4.52 | 43.5857 |
| M/Pancreas/PRI 2 | 32.48 | 26.33 | 6.15 | 14.082 |
| M/Kidney/Normal/MPI 88 | 30.23 | 26.84 | 3.38 | 96.0547 |
| M/Kidney/Normal/MPI 282 | 29.34 | 25.94 | 3.4 | 95.0612 |
| HUVEC Vehicle | 29.25 | 21.45 | 7.8 | 4.4871 |
| HUVEC Mev | 28.16 | 20.87 | 7.29 | 6.3899 |
| HAEC Vehicle | 28.14 | 21.16 | 6.97 | 7.9491 |
| HAEC Mev | 29.61 | 22.66 | 6.95 | 8.088 |

In addition, a human vessel panel was tested, which revealed high expression of human 67102 mRNA in normal artery, HUVEC, coronary smooth muscle cells, diseased aorta, and normal hypothalamus (see Table, 6, below).

TABLE 6

| Tissue Type | Mean | β 2 Mean | ∂∂ Ct | Expression |
|---|---|---|---|---|
| Artery normal | 27.32 | 21.75 | 5.57 | 21.0505 |
| Aorta diseased | 28.27 | 21.71 | 6.55 | 10.6353 |
| Vein normal | 30.38 | 19.83 | 10.55 | 0.6693 |
| Coronary SMC | 28.61 | 22.23 | 6.38 | 12.0485 |
| HUVEC | 26.32 | 20.32 | 6 | 15.5709 |
| Hemangioma | 25.91 | 19.07 | 6.83 | 8.7895 |
| Heart normal | 27.16 | 19.98 | 7.17 | 6.9441 |
| Heart CHF | 27.2 | 19.06 | 8.14 | 3.545 |
| Kidney | 25.54 | 19.59 | 5.96 | 16.12 |
| Skeletal Muscle | 30.52 | 21.5 | 9.03 | 1.9196 |
| Adipose normal | 30.11 | 19.95 | 10.15 | 0.8771 |
| Pancreas | 29.57 | 21.23 | 8.33 | 3.1076 |
| primary osteoblasts | 28.09 | 19.85 | 8.23 | 3.3191 |
| Osteoclasts (diff) | 29.79 | 17.02 | 12.77 | 0.1432 |
| Skin normal | 29.31 | 21.41 | 7.89 | 4.2011 |
| Spinal cord normal | 28.3 | 20.36 | 7.93 | 4.0863 |
| Brain Cortex normal | 28.25 | 22.04 | 6.21 | 13.5084 |
| Brain Hypothalamus normal | 28.93 | 21.49 | 7.44 | 5.7589 |
| Nerve | 28.34 | 21.3 | 7.04 | 7.5989 |
| DRG (Dorsal Root Ganglion) | 29.16 | 21.11 | 8.04 | 3.7994 |
| Breast normal | 27.81 | 20.47 | 7.34 | 6.1508 |
| Breast tumor | 29.08 | 20.41 | 8.68 | 2.4466 |
| Ovary normal | 26.44 | 19.7 | 6.74 | 9.3878 |
| Ovary Tumor | 30.93 | 19.6 | 11.34 | 0.3871 |
| Prostate Normal | 28.11 | 19.48 | 8.63 | 2.5241 |
| Prostate Tumor | 27.68 | 19.68 | 8 | 3.9063 |
| Salivary glands | 28.9 | 19.18 | 9.71 | 1.194 |
| Colon Tumor | 27.98 | 18.82 | 9.16 | 1.742 |
| Lung normal | 26.96 | 17.4 | 9.56 | 1.3202 |
| Lung tumor | 27.82 | 19.64 | 8.19 | 3.4361 |
| Lung COPD | 26.38 | 17.66 | 8.72 | 2.3633 |
| Colon IBD | 28.27 | 17.29 | 10.98 | 0.4934 |
| Liver normal | 29.14 | 19.58 | 9.56 | 1.3248 |
| Liver fibrosis | 29.89 | 21.08 | 8.8 | 2.2358 |
| Spleen normal | 26.95 | 19.09 | 7.86 | 4.3193 |
| Tonsil normal | 25.01 | 16.8 | 8.21 | 3.3654 |
| Lymph node normal | 26.3 | 18.22 | 8.09 | 3.6828 |
| Small intestine normal | 29.03 | 19.59 | 9.45 | 1.4347 |
| Skin-Decubitus | 27.66 | 20.32 | 7.34 | 6.1722 |
| Synovium | 28.22 | 19.23 | 8.98 | 1.9804 |
| BM-MNC | 29.57 | 18.46 | 11.12 | 0.4509 |
| Activated PBMC | 28.38 | 17.25 | 11.14 | 0.4447 |
| Neutrophils | 27.43 | 18.4 | 9.04 | 1.8997 |
| Megakaryocytes | 26.72 | 17.88 | 8.84 | 2.1822 |
| Erythroid | 31.52 | 21.26 | 10.26 | 0.8183 |
| Colon normal | 30.07 | 19.25 | 10.82 | 0.5551 |

Tissue Distribution of Human 44181

A human vessel panel was tested revealing highest expression of human 44181 mRNA in LSS HUVEC (see Table 7, below).

TABLE 7

| Tissue Type | Mean | β 2 Mean | ∂∂ Ct | Expression |
|---|---|---|---|---|
| Static Huvec | 25.37 | 19.18 | 6.19 | 13.697 |
| LSS Huvec | 25.7 | 20.02 | 5.68 | 19.4377 |
| Aortic SMC | 28.75 | 20.32 | 8.43 | 2.9095 |
| Coronary SMC | 28.52 | 21.2 | 7.31 | 6.3019 |
| H/Adipose/MET 9 | 36.07 | 18.41 | 17.66 | 0 |
| Diseased Heart/PIT 1 | 29.28 | 21.15 | 8.13 | 3.5697 |
| H/Artery/Normal/Carotid/CLN | 37.9 | 18.32 | 19.59 | 0 |
| 595 | | | | |
| H/Artery/Normal/Carotid/CLN | 39.97 | 19.49 | 20.48 | 0 |
| 598 | | | | |
| H/Artery/normal/NDR 352 | 40 | 20.2 | 19.8 | 0 |
| H/Artery/Normal/AMC 150 | 40 | 22.27 | 17.73 | 0 |
| H/Artery/Normal/AMC 73 | 40 | 23.84 | 16.16 | 0 |
| IMA/AMC 247 | 39.73 | 22.79 | 16.95 | 0 |
| IMA/AMC 254 | 33.79 | 22.23 | 11.56 | 0.3324 |
| IMA/AMC 259 | 33.68 | 21.12 | 12.56 | 0.1656 |
| IMA/AMC 261 | 34.73 | 21.23 | 13.5 | 0.0863 |
| IMA/AMC 275 | 40 | 24.52 | 15.48 | 0 |
| IMA/AMC 279 | 30.89 | 22.41 | 8.48 | 0 |
| H/Artery/Diseased/iliac/NDR | 36.59 | 18.43 | 18.16 | 0 |
| 753 | | | | |
| H/Artery/Diseased/Tibial/PIT | 40 | 19.84 | 20.16 | 0 |
| 679 | | | | |
| Aorta/Diseased/PIT 732 | 34.74 | 21.32 | 13.41 | 0.0916 |
| Aorta/Diseased/PIT 710 | 33.04 | 22.48 | 10.56 | 0.6624 |
| Aorta/Diseased/PIT 711 | 31.89 | 22.09 | 9.8 | 1.1218 |
| Aorta/Diseased/PIT 712 | 32.92 | 22.09 | 10.84 | 0.5474 |
| H/Vein/Normal/Saphenous/ | 32.66 | 16.82 | 15.83 | 0.0172 |
| NDR 721 | | | | |
| H/Vein/Normal/SaphenousAMC | 40 | 20 | 20 | 0 |
| 107 | | | | |
| H/Vein/Normal/NDR 239 | 40 | 20.61 | 19.39 | 0 |
| H/Vein/Normal/Saphenous/ | 40 | 19.1 | 20.9 | 0 |
| NDR 237 | | | | |
| H/Vein/Normal/NDR 235 | 40 | 21.34 | 18.66 | 0 |
| H/Vein/Normal/MPI 1101 | 33.56 | 19.59 | 13.98 | 0.0621 |
| HMVEC/Vehicle/24 hr | 30.04 | 17.84 | 12.2 | 0.2125 |
| HMVEC/Mev/24 hr/1X | 29.77 | 18 | 11.76 | 0.2883 |
| HMVEC/MEV/24 HR/2.5X | 30.32 | 18.67 | 11.65 | 0.3112 |
| HMVEC/MEV/48 HR/1X | 31.58 | 18.8 | 12.79 | 0.1417 |
| HMVEC/MEV/48 HR/2.5X | 31.77 | 18.37 | 13.4 | 0.0922 |
| HUVEC/Vehicle/24 hr | 30.5 | 18.15 | 12.36 | 0.1909 |
| HUVEC/Mev/24 hr/1X | 30.28 | 17.52 | 12.76 | 0.1442 |
| HUVEC/MEV/24 HR/2.5X | 29.35 | 19.18 | 10.18 | 0.865 |
| HUVEC/MEV/48 HR/1X | 35.68 | 21.54 | 14.14 | 0 |
| HUVEC/MEV/48 HR/2.5X | 34.7 | 23 | 11.7 | 0.3016 |

Tissue Distribution of Human 67084

A human vessel panel was tested revealing highest expression of human 67084 mRNA in HUVEC, LSS HUVEC, and coronary smooth muscle cells (see Table 8, below).

TABLE 8

| Tissue Type | Mean | β 2 Mean | ∂∂ Ct | Expression |
|---|---|---|---|---|
| Aortic SMC | 25.92 | 19.23 | 6.7 | 9.6517 |
| Coronary SMC | 26.59 | 20.36 | 6.23 | 13.3224 |
| Huvec Static | 23.39 | 18.5 | 4.88 | 33.843 |
| Huvec LSS | 24.31 | 18.32 | 5.99 | 15.7883 |
| H/Adipose/MET 9 | 26.4 | 18.46 | 7.94 | 4.0721 |
| H/Artery/Normal/Carotid/CLN | 26.83 | 18.84 | 8 | 3.9198 |
| 595 | | | | |
| H/Artery/Normal/Carotid/CLN | 28.49 | 20.16 | 8.34 | 3.0968 |
| 598 | | | | |
| H/Artery/normal/NDR 352 | 27.12 | 20.32 | 6.8 | 8.9432 |
| H/IM Artery/Normal/AMC 73 | 31.48 | 23.36 | 8.12 | 3.607 |
| H/Muscular Artery/Normal/ | 30.93 | 23.56 | 7.38 | 6.0243 |
| AMC 236 | | | | |
| H/Muscular Artery/Normal/ | 33.77 | 24.84 | 8.92 | 2.0645 |
| AMC 247 | | | | |
| H/Muscular Artery/Normal/ | 30.69 | 23.68 | 7 | 7.7855 |
| AMC 254/ | | | | |
| H/Muscular Artery/Normal/ | 29.9 | 22.12 | 7.78 | 4.5497 |
| AMC 259 | | | | |
| H/Muscular Artery/Normal/ | 29.93 | 21.13 | 8.8 | 2.2436 |
| AMC 261 | | | | |
| H/Muscular Artery/Normal/ | 30.29 | 22.97 | 7.33 | 6.2367 |
| AMC 275 | | | | |
| H/Aorta/Diseased/PIT 732 | 29.02 | 21.35 | 7.67 | 4.8932 |
| H/Aorta/Diseased/PIT 710 | 31.36 | 22.8 | 8.56 | 2.6496 |
| H/Aorta/Diseased/PIT 711 | 31.31 | 22.6 | 8.71 | 2.3963 |
| H/Aorta/Diseased/PIT 712 | 31.4 | 22.48 | 8.92 | 2.0645 |
| H/Artery/Diseased/iliac/NDR | 25.37 | 17.73 | 7.64 | 4.996 |
| 753 | | | | |
| H/Artery/Diseased/Tibial/PIT | 28.55 | 19.45 | 9.11 | 1.816 |
| 679 | | | | |
| H/Vein/Normal/SaphenousAMC | 29.48 | 21.11 | 8.38 | 3.0121 |
| 107 | | | | |
| H/Vein/Normal/Saphenous/NDR | 28.67 | 19.86 | 8.8 | 2.2358 |
| 237 | | | | |
| H/Vein/Normal/PIT 1010 | 28.31 | 20.55 | 7.76 | 4.5973 |
| H/Vein/Normal/AMC 191 | 29.25 | 20.77 | 8.47 | 2.8104 |
| H/Vein/Normal/AMC 130 | 28.32 | 20.45 | 7.88 | 4.2598 |
| H/Vein/Normal/AMC 188 | 31.68 | 24.61 | 7.06 | 7.4943 |
| H/Vein/Normal/NDR 239 | 35.65 | 29.23 | 6.42 | 0 |
| HUVEC Vehicle | 26.86 | 20.14 | 6.71 | 9.5188 |
| HUVEC Mev | 25.83 | 18.52 | 7.3 | 6.3238 |
| HAEC Vehicle | 26.57 | 19.64 | 6.94 | 8.1443 |
| HAEC Mev | 27.81 | 21.13 | 6.67 | 9.7864 |

### Example 3

### Expression of Recombinant 67076, 67102, 44181, 67084FL, or 67084alt Polypeptide in Bacterial Cells

In this example, human 8099, 46455, 54414, 53763, 67076, 67102, 44181, 67084FL, or 67084alt is expressed as a recombinant glutathione-S-transferase (GST) fusion polypeptide in E. coli and the fusion polypeptide is isolated and characterized. Specifically, 8099, 46455, 54414, 53763, 67076, 67102, 44181, 67084FL, or 67084alt is fused to GST and this fusion polypeptide is expressed in E. coli, e.g., strain PEB199. Expression of the GST-PLTR fusion polypeptide in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 4

### Expression of Recombinant 8099, 46455, 54414, 53763, 67076, 67102, 44181, 67084FL, or 67084alt Polypeptides in COS Cells

To express the human 8099, 46455, 54414, 53763, 67076, 67102, 44181, 67084FL, or 67084alt gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire PLTR polypeptide and an HA tag (Wilson et al. (1984) Cell 37:767) or a FLAG tag fused in-frame to its 3′ end of the fragment is cloned into the polylinker region of the vector, thereby placing the expression of the recombinant polypeptide under the control of the CMV promoter.

To construct the plasmid, the human 8099, 46455, 54414, 53763, 67076, 67102, 44181, 67084FL, or 67084alt DNA sequence is amplified by PCR using two primers. The 5′ primer contains the restriction site of interest followed by approximately twenty nucleotides of the 8099, 46455, 54414, 53763, 67076, 67102, 44181, 67084FL, or 67084alt coding sequence starting from the initiation codon; the 3′ end sequence contains complementary sequences to the other restriction site of interest, a translation stop codon, the HA tag or FLAG tag and the last 20 nucleotides of the 8099, 46455, 54414, 53763, 67076, 67102, 44181, 67084FL, or 67084alt coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 8099, 46455, 54414, 53763, 67076, 67102, 44181, 67084FL, or 67084alt gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the human 8099, 46455, 54414, 53763, 67076, 67102, 44181, 67084FL, or 67084alt-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the IC54420 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the human 8099, 46455, 54414, 53763, 67076, 67102, 44181, 67084FL, or 67084alt coding sequence is cloned directly into the polylinker of the pCDNA/Amp vector using the appropriate restriction sites. The resulting plasmid is transfected into COS cells in the manner described above, and the expression of the 8099, 46455, 54414, 53763, 67076, 67102, 44181, 67084FL, or 67084alt polypeptide is detected by radiolabelling and immunoprecipitation using a 8099, 46455, 54414, 53763, 67076, 67102, 44181, 67084FL, or 67084alt-specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule selected from the group consisting of:
(a) a nucleic acid molecule comprising the nucleotide sequence set forth in SEQ ID NO:1, 4, 14, 21, 28, 31, 34, 37, 40, 43, 52, 55, or a complement thereof; and
(b) a nucleic acid molecule comprising the nucleotide sequence set forth in SEQ ID NO:3, 6, 16, 23, 30, 33, 36, 39, 42, 45, 54, 57, or a complement thereof.

2. An isolated nucleic acid molecule which encodes a polypeptide comprising the amino acid sequence set forth in SEQ ID NO:2, 5, 15, 22, 29, 32, 35, 38, 41, 44, 53, or 56, or a complement thereof.

3. An isolated nucleic acid molecule comprising the nucleotide sequence contained in the insert of the plasmid deposited with ATCC® as Accession Number ______, ______, ______, ______, ______, ______, ______, ______, ______, ______, ______, or ______.

4. An isolated nucleic acid molecule which encodes a polypeptide comprising the amino acid sequence set forth in SEQ ID NO: 2, 5, 15, 22, 29, 32, 35, 38, 41, 44, 53, or 56, or a complement thereof.

5. An isolated nucleic acid molecule selected from the group consisting of:
(a) a nucleic acid molecule comprising a nucleotide sequence which is at least 60% identical to the nucleotide sequence of SEQ ID NO:1, 3, 4, 6, 14, 16, 21, 23, 28, 30, 31, 33, 34, 36, 37, 39, 40, 42, 43, 45, 52, 54, 55, 57, or a complement thereof;
(b) a nucleic acid molecule comprising a fragment of at least 30 nucleotides of a nucleic acid comprising the nucleotide sequence of SEQ ID NO: 1, 3, 4, 6, 14, 16, 21, 23, 28, 30, 31, 33, 34, 36, 37, 39, 40, 42, 43, 45, 52, 54, 55, 57, or a complement thereof;
(c) a nucleic acid molecule which encodes a polypeptide comprising an amino acid sequence at least about 60% identical to the amino acid sequence of SEQ ID NO: 2, 5, 15, 22, 29, 32, 35, 38, 41, 44, 53, or 56, or a complement thereof; and
(d) a nucleic acid molecule which encodes a fragment of a polypeptide comprising the amino acid sequence of SEQ ID NO: 2, 5, 15, 22, 29, 32, 35, 38, 41, 44, 53, or 56, wherein the fragment comprises at least 10 contiguous amino acid residues of the amino acid sequence of SEQ ID NO: 2, 5, 15, 22, 29, 32, 35, 38, 41, 44, 53, or 56, or a complement thereof.

6. An isolated nucleic acid molecule comprising the nucleic acid molecule of any one of claims 1, 2, 3, 4, or 5, and a nucleotide sequence encoding a heterologous polypeptide.

7. A vector comprising the nucleic acid molecule of any one of claims 1, 2, 3, 4, or 5.

8. The vector of claim 7, which is an expression vector.

9. A host cell transfected with the expression vector of claim 8.

10. A method of producing a polypeptide comprising culturing the host cell of claim 9 in an appropriate culture medium to, thereby, produce the polypeptide.

11. An isolated polypeptide selected from the group consisting of:
a) a polypeptide comprising the amino acid sequence of SEQ ID NO:2, 5, 15, 22, 29, 32, 35, 38, 41, 44, 53, or 56;
b) a polypeptide consisting of the amino acid sequence of SEQ ID NO:2, 5, 15, 22, 29, 32, 35, 38, 41, 44, 53, or 56;
c) a fragment of a polypeptide comprising the amino acid sequence of SEQ ID NO: 2, 5, 15, 22, 29, 32, 35, 38, 41, 44, 53, or 56, wherein the fragment comprises at least 10 contiguous amino acids of SEQ ID NO: 2, 5, 15, 22, 29, 32, 35, 38, 41, 44, 53, or 56;
d) a naturally occurring allelic variant of a polypeptide comprising the amino acid sequence of SEQ ID NO: 2, 5, 15, 22, 29, 32, 35, 38, 41, 44, 53, or 56, wherein the polypeptide is encoded by a nucleic acid molecule which hybridizes to complement of a nucleic acid molecule consisting of SEQ ID NO: 1, 3, 4, 6, 14, 16, 21, 23, 28, 30, 31, 33, 34, 36, 37, 39, 40, 42, 43, 45, 52, 54, 55, 57 under stringent conditions;
e) a polypeptide which is encoded by a nucleic acid molecule comprising a nucleotide sequence which is at least 60% identical to a nucleic acid comprising the nucleotide sequence of SEQ ID NO: 1, 3, 4, 6, 14, 16, 21, 23, 28, 30, 31, 33, 34, 36, 37, 39, 40, 42, 43, 45, 52, 54, 55, 57; and
f) a polypeptide comprising an amino acid sequence which is at least 60% identical to the amino acid sequence of SEQ ID NO: 2, 5, 15, 22, 29, 32, 35, 38, 41, 44, 53, or 56.

12. The polypeptide of claim 11, further comprising heterologous amino acid sequences.

13. An antibody which selectively binds to a polypeptide of claim 11.

14. A method for detecting the presence of a polypeptide of claim 11 in a sample comprising:
a) contacting the sample with a compound which selectively binds to the polypeptide; and
b) determining whether the compound binds to the polypeptide in the sample to thereby detect the presence of a polypeptide of claim 11 in the sample.

15. The method of claim 14, wherein the compound which binds to the polypeptide is an antibody.

16. A kit comprising a compound which selectively binds to a polypeptide of claim 13 and instructions for use.

17. A method for detecting the presence of a nucleic acid molecule of any one of claims 1, 2, 3, 4, or 5 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of any one of claims 1, 2, 3, 4, or 5 in the sample.

18. The method of claim 17, wherein the sample comprises mRNA molecules and is contacted with a nucleic acid probe.

19. A kit comprising a compound which selectively hybridizes to a nucleic acid molecule of any one of claims 1, 2, 3, 4, or 5 and instructions for use.

20. A method for identifying a compound which binds to a polypeptide of claim 13 comprising:
a) contacting the polypeptide, or a cell expressing the polypeptide with a test compound; and
b) determining whether the polypeptide binds to the test compound.

21. The method of claim 20, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for TWIK-6, TWIK-7, IC23927, TWIK-8, IC47611, IC47615, HNMDA-1, TWIK-9, α₂δ-4, 54414, or 53763 activity.

22. A method for modulating the activity of a polypeptide of claim 13 comprising contacting the polypeptide or a cell expressing the polypeptide with a compound which binds to the polypeptide in a sufficient concentration to modulate the activity of the polypeptide.

23. A method for identifying a compound which modulates the activity of a polypeptide of claim 11 comprising:
a) contacting a polypeptide of claim 11 with a test compound; and
b) determining the effect of the test compound on the activity of the polypeptide to thereby identify a compound which modulates the activity of the polypeptide.
